(19) United States
(12) Patent Application Publication
(10) Pub. No.: US 2026/0015360 A1
(43) Pub. Date: Jan. 15, 2026

(54) BIARYL DERIVATIVES AND RELATED USES

(71) Applicant: MOMA Therapeutics, Inc., Cambridge, MA (US)

(72) Inventors: Michael H. Reutershan, Acton, MA (US); Laurie B. Schenkel, Belmont, MA (US); Meredeth A. McGowan, Bedford, MA (US); Timothy J. Guzi, Sudbury, MA (US); John R. Butler, Thousand Oaks, CA (US); Haoxuan Wang, Somerville, MA (US); Cen Gao, Carmel, IN (US); Xin Yan, Newton Highlands, MA (US); Yonghong Bai, Lexington, MA (US)

(21) Appl. No.: 19/331,736

(22) Filed: Sep. 17, 2025

Related U.S. Application Data
(63) Continuation of application No. PCT/US2024/023442, filed on Apr. 5, 2024.
(60) Provisional application No. 63/457,293, filed on Apr. 5, 2023.
(60) Provisional application No. 63/539,045, filed on Sep. 18, 2023.

(51) Int. Cl. C07D 491/107; A61K 31/437; A61K 31/4439; A61K 31/444; A61K 31/4545; A61K 31/4725; A61K 31/496; A61K 31/4985; A61K 31/501; A61K 31/5025; A61K 31/506; A61K 31/519; A61K 31/5377; A61K 31/538; A61K 31/5383; A61K 31/5386; C07D 413/12; C07D 417/12; C07D 417/14; C07D 471/04; C07D 498/08; C07D 513/04; C07D 519/00

(52) U.S. Cl. CPC C07D 491/107; A61K 31/437; A61K 31/4439; A61K 31/444; A61K 31/4545; A61K 31/4725; A61K 31/496; A61K 31/4985; A61K 31/501; A61K 31/5025; A61K 31/506; A61K 31/519; A61K 31/5377; A61K 31/538; A61K 31/5383; A61K 31/5386; C07D 413/12; C07D 417/12; C07D 417/14; C07D 471/04; C07D 498/08; C07D 513/04; C07D 519/00

(57) ABSTRACT

The present disclosure relates to compounds of Formula (I):

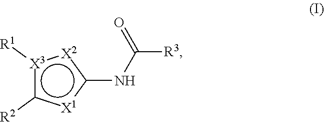

and to their prodrugs, pharmaceutically acceptable salts, pharmaceutical compositions, methods of use, and methods for their preparation. The compounds disclosed herein are useful for modulating DNA polymerase Θ activity and may be used in the treatment of disorders in which DNA polymerase Θ activity is implicated, such as cancer.

### RELATED APPLICATIONS

This application is a continuation of International Patent Application No. PCT/US2024/023442, filed Apr. 5, 2024, which claims priority to, and the benefit of, U.S. Provisional Application Nos. 63/457,293, filed on Apr. 5, 2023, and 63/539,045, filed on Sep. 18, 2023, the entire contents of which are incorporated herein by reference.

### BACKGROUND

The present disclosure relates to small molecule antagonists of DNA polymerase Θ, designed for the treatment of cancer.

The proper repair of DNA double-strand breaks (DSBs) is essential for the maintenance of genome integrity. Inaccurate repair of DSBs can cause mutations in crucial coding or regulatory regions, while the accumulation of unrepaired DNA damage introduces mitotic stress that can lead to genomic alterations or cell death. In normal cells, DSBs are primarily repaired through two key mechanisms. DNA lesions that occur during DNA replication (S-phase) are typically repaired by homologous recombination (HR), which uses a replicated “sister chromatid” as a template for error-free repair. In contrast, non-homologous end-joining (NHEJ) is the primary repair process for DSBs when there is no DNA template available for templated repair. A third DSB repair pathway, termed alternative end-joining (Alt-EJ), microhomology-mediated end-joining (MMEJ), or theta-mediated end-joining (TMEJ), is executed by polymerase-theta (POLΘ). In contrast to NHEJ and HR, TMEJ is thought to play a limited role in heathy cells under normal conditions.

Some tumors harbor inactivating mutations in homologous repair genes, most commonly disabling the function of BRCA1 or BRCA2. As a result, these tumors are intrinsically sensitive to DNA damaging agents, as well as to inhibitors of specific DNA repair proteins. Accordingly, DNA cross-linking agents such as platinum-based chemotherapies are more effective in BRCA-mutant tumors than in tumors with intact BRCA function. Similarly, small molecule inhibitors of PARP1/2 are efficacious in BRCA1/2-deficient tumors, which rely on the PARP enzymes to repair single-strand DNA breaks and prevent their conversion to toxic DSBs that overwhelm the repair capacity of HR-deficient cells.

An alternative approach to therapeutically targeting HR-deficient tumors is through the inhibition of back-up repair pathways such as POLΘ-mediated TMEJ. Consistent with its role as a back-up DNA repair enzyme, ablation of the POLO gene locus is well tolerated in mouse models, causing only mild phenotypes characterized by micronuclei in reticulocytes and increased cellular (but not organismic) sensitivity to DNA cross-linking agents. In contrast, POLO DNA repair activity has been shown to be essential for cell survival when NHEJ or HR are inactivated, implicating POLO as a potential target for cancer therapy in specific mutational settings.

Unique in the human genome, POLO contains both an N-terminal SF2 DNA helicase domain and a C-terminal DNA polymerase domain. In the setting of chromosomal DSBs, these domains work in tandem to repair DSBs containing long 3′-single-strand DNA overhangs. Specifically, the helicase domain is thought to strip the RPA protein complex from the overhangs and promote annealing to the opposite DNA end via a region of DNA microhomology. The annealed DNA then serves as a primer for the POLO polymerase domain, which extends the annealed DNA to fill in the single-strand DNA gap. Given the role of POLO catalytic activity in DNA repair processes that are crucial in HR-deficient tumors, POLO represents an attractive target for the development of chemical inhibitors to exploit newly discovered functional dependencies.

The disclosure arises from a need to provide compounds for the modulation of DNA polymerase Θ activity with improved therapeutic potential. In particular, compounds with improved physicochemical, pharmacological and/or pharmaceutical properties.

### SUMMARY

In some aspects, the present disclosure provides a compound of Formula (I):

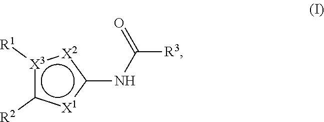

or a pharmaceutically acceptable salt, solvate, clathrate, hydrate, stereoisomer, or tautomer thereof, wherein:
X¹ is CH, S, or N;
X² is N, S, or O;
X³ is C or N;
R¹ and R², together with the atoms to which they are attached, form a C₆-C₁₀ aryl or 5- to 10-membered heteroaryl, wherein the C₆-C₁₀ aryl or 5- to 10-membered heteroaryl is optionally substituted with one or more R^{a};
each R^{a} independently is oxo, halo, cyano, —OH, —NH₂, —C(O)N(R^{b})(R^{c}), —N(R^{b})C(O)(R^{c}), C₁-C₆ alkyl, C₂-C₆ alkenyl, C₂-C₆ alkynyl, C₁-C₆ haloalkyl, C₃-C₁₀ cycloalkyl, C₄-C₁₀ cycloalkenyl, C₆-C₁₀ aryl, 5- to 10-membered heteroaryl, 3- to 10-membered heterocycloalkyl, or 4- to 10-membered heterocycloalkenyl, wherein the C₁-C₆ alkyl, C₂-C₆ alkenyl, C₂-C₆ alkynyl, C₁-C₆ haloalkyl, C₃-C₁₀ cycloalkyl, C₄-C₁₀ cycloalkenyl, C₆-C₁₀ aryl, 5- to 10-membered heteroaryl, 3- to 10-membered heterocycloalkyl, or 4- to 10-membered heterocycloalkenyl is optionally substituted with one or more R^{a1};
each R^{b} and R^{c} independently is H, C₁-C₆ alkyl, C₃-C₁₀ cycloalkyl, or 3- to 10-membered heterocycloalkyl, wherein the C₁-C₆ alkyl, C₃-C₁₀ cycloalkyl, or 3- to 10-membered heterocycloalkyl is optionally substituted with one or more —OH or C₁-C₆ haloalkyl; or
R^{b} and R^{c}, together with the atoms to which they are attached, form a 3- to 10-membered heterocycloalkyl, wherein the 3- to 10-membered heterocycloalkyl is optionally substituted with one or more —OH, —O(C₁-C₆ haloalkyl), —O(C₁-C₆ alkyl), —C(O)(O—(C₁-C₆ alkyl)), C₁-C₆ alkyl, or —N(C₁-C₆ alkyl)₂;
each R^{a1} independently is oxo, halo, cyano, —OH, —O(C₃-C₁₀ cycloalkyl), —NH₂, —NH(C₁-C₆ alkyl), —N(C₁-C₆ alkyl)₂, —C(O)OH, —C(O)O(C₁-C₆ alkyl), C₁-C₆ alkyl, C₂-C₆ alkenyl, C₂-C₆ alkynyl, C₁-C₆ haloalkyl, C₁-C₆ alkoxy, C₁-C₆ haloalkoxy, —O—(C₃-C₁₀ cycloalkyl), C₃-C₁₀ cycloalkyl, 3- to 10-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl is optionally substituted with one or more R^{a2};
each R^{a2} independently is oxo, halo, cyano, —OH, or —NH₂; R³ is C₆-C₁₀ aryl or 6- to 10-membered heteroaryl, wherein the C₆-C₁₀ aryl or 6- to 10-membered heteroaryl is substituted with one or more R^{3a};
each R^{3a} independently is halo, cyano, —OH, —NH₂, C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, —O(C₆-C₁₀ aryl), C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, —O(C₆-C₁₀ aryl), C₆-C₁₀ aryl, or 5- to 10-membered heteroaryl is optionally substituted with one or more R^{3a1}; and
each R^{3a1} independently is oxo, halo, cyano, —NH₂, —NH—C(O)O(C₁-C₆ alkyl), —C(O)(C₁-C₆ alkyl), —C(O)NH₂, —C(O)(O—(C₁-C₆ alkyl)), C₁-C₆ alkyl optionally substituted with one or more C₃-C₁₀ cycloalkyl, C₂-C₆ alkenyl, C₂-C₆ alkynyl, C₁-C₆ haloalkyl, C₁-C₆ alkoxy, —O(C₁-C₆ haloalkyl), C₃-C₁₀ cycloalkyl, C₆-C₁₀ aryl, or 5- to 10-membered heteroaryl;
wherein, when R^{3a} is halo and R¹ and R², together with the atoms to which they are attached, form a 6-membered heteroaryl then R^{a} is not —CF₃.

In some embodiments, X¹ is CH or N; and R¹ and R², together with the atoms to which they are attached, form a pyridine optionally substituted with one or more R^{a}.

In some embodiments, X² is N or O; and R¹ and R², together with the atoms to which they are attached, form a pyridine optionally substituted with one or more R^{a}.

In some embodiments, when R¹ and R², together with the atoms to which they are attached, form a pyridine optionally substituted with one or more R^{a}, then X¹ is not S.

In some embodiments, when R¹ and R², together with the atoms to which they are attached, form a pyridine optionally substituted with one or more R^{a}, then X² is not S.

In some embodiments, when R¹ and R², together with the atoms to which they are attached, form a pyridine optionally substituted with one or more R^{a}, then

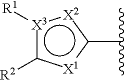

is not

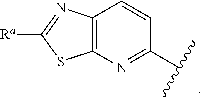

In some embodiments, when R^{a} is

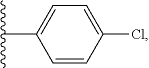 [SMILES: *c1ccc(Cl)cc1]

then R³ is not

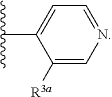

In some embodiments, when R^{a} is

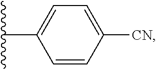 [SMILES: *c1ccc(C#N)cc1]

then R³ is not

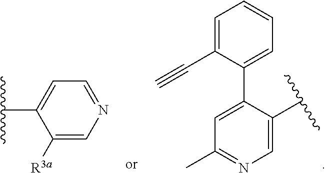

In some embodiments, when R¹ and R², together with the atoms to which they are attached, form a pyrazine substituted with one or more R^{a}, and R^{a} is phenyl substituted with one or more R^{a1}, then R^{a1} is not —Cl, —F, —CN, —O(CH₃), —O(CF₃), —O(CHF₂), —CH₃, or cyclopropyl.

In some embodiments, when R¹ and R², together with the atoms to which they are attached, form a pyrazine substituted with one or more R^{a}, and R^{a} is pyridine substituted with one or more R^{a1}, then R^{a1} is not —Cl, —CN, —CF₃, —O(CH₃), —O(CHF₂), cyclopropyl, —C(CH₃)₂(CN), or —C(CH₃)₂(OH).

In some embodiments, when R¹ and R², together with the atoms to which they are attached, form a pyrazine substituted with one or more R^{a}, then R^{a} is not

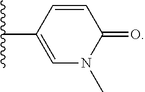 [SMILES: *c1ccc(=O)n(C)c1]

In some embodiments, the compound of Formula (I) is not 5′-methoxy-2′,6-dimethyl-N-(6-(tetrahydrofuran-3-yl)thiazolo[4,5-b]pyrazin-2-yl)-[4,4′-bipyridine]-3-carboxamide.

In some aspects, the present disclosure provides a compound obtainable by, or obtained by, a method for preparing a compound as described herein (e.g., a method comprising one or more steps described in Schemes 1-11).

In some aspects, the present disclosure provides a pharmaceutical composition comprising a compound of the present disclosure, or a pharmaceutically acceptable salt thereof, and a pharmaceutically acceptable diluent or carrier.

In some aspects, the present disclosure provides an intermediate as described herein, being suitable for use in a method for preparing a compound as described herein (e.g., the intermediate is selected from the intermediates described in Examples 1-451).

In some aspects, the present disclosure provides a method of modulating DNA polymerase Θ activity (e.g., in vitro or in vivo), comprising contacting a cell with an effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in modulating DNA polymerase Θ activity (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides use of a compound of the present disclosure or a pharmaceutically acceptable salt thereof in the manufacture of a medicament for modulating DNA polymerase Θ activity (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein. In some aspects, modulation is inhibition.

In some aspects, the present disclosure provides a method of preparing a compound of the present disclosure.

In some aspects, the present disclosure provides a method of preparing a compound, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

The present disclosure relates to biaryl derivatives, prodrugs, and pharmaceutically acceptable salts thereof, which may modulate DNA polymerase Θ activity and are accordingly useful in methods of treatment of the human or animal body. The present disclosure also relates to processes for the preparation of these compounds, to pharmaceutical compositions comprising them and to their use in the treatment of disorders in which DNA polymerase Θ is implicated, such as cancer.

### Definitions

Unless otherwise stated, the following terms used in the specification and claims have the following meanings set out below.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options. For example, while various options for variables X¹, X², X³, R¹, R², R^{a}, R^{b}, R^{c}, R^{a1}, R^{a2}, R³, R^{3a}, and R^{3a1} are described herein, the disclosure may be interpreted as excluding structures for non-operable compound caused by certain combinations of variables X¹, X², X³, R¹, R², R^{a}, R^{b}, R^{c}, R^{a1}, R^{a2}, R³, R^{3a}, and R^{3a1}.

As used herein, “alkyl”, “Cl, C₂, C₃, C₄, C₅ or C₆ alkyl” or “C₁-C₆ alkyl” is intended to include Cl, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl includes Cl, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl.

In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, 5,6-dihydro-4H-cyclopenta[b]thiophenyl, and the like. In the case of multicyclic heterocycloalkyl, only one of the rings in the heterocycloalkyl needs to be non-aromatic (e.g., 4,5,6,7-tetrahydrobenzo[c]isoxazolyl).

It is understood that when a variable has two attachments to the rest of the formula of the compound, the two attachments could be at the same atom or different atoms of the variable.

For example, when a variable (e.g., variable X) is cycloalkyl or heterocycloalkyl, and has two attachments to the rest of the formula of the compound, the two attachments could be at the same atom or different atoms of the cycloalkyl or heterocycloalkyl.

As used herein, the term “aryl” includes groups with aromaticity, including “conjugated,” or multicyclic systems with one or more aromatic rings and do not contain any heteroatom in the ring structure. The term aryl includes both monovalent species and divalent species.

Examples of aryl groups include, but are not limited to, phenyl, biphenyl, naphthyl and the like.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, isothiazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl).

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

When a ring is denoted with a circle and all ring members are carbon atoms

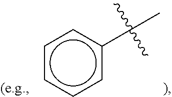

the ring is an aryl ring. When a ring is denoted with a circle and comprises at least one ring member that is a heteroatom

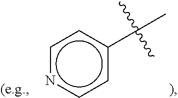

the ring is a heteroaryl ring. Multicyclic aryl or heteroaryl groups can be denoted with two or more circles

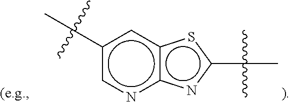

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “about” refers to a recited amount, value, or duration ±10% or less of said amount, value, or duration. In some embodiments, “about” refers to a recited amount, value, or duration ±10%, ±8%, ±6%, ±5%, ±4%, ±2%, ±1%, or ±0.5%. In other embodiments, “about” refers to a recited amount, value, or duration ±10%, ±8%, 6%, ±5%, ±4%, or ±2%. In other embodiments, “about” refers to a recited amount, value, or duration ±5%. In some embodiments, “about” refers to a listed amount, value, or duration 2% or ±1%. For example, in some embodiments, when the term “about” is used when reciting a temperature or temperature range, these terms refer to the recited temperature or temperature range ±5° C., ±2° C., or ±1° C. In other embodiments, the term “about” refers to the recited temperature or temperature range ±2° C.

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula.

Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O—.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field.

Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for organic Synthesis, John Wiley and Sons (1994); and L. Paquette, ed., Encyclopedia of Reagents for organic Synthesis, John Wiley and Sons (1995), incorporated by reference herein, are useful and recognized reference textbooks of organic synthesis known to those in the art.

One of ordinary skill in the art will note that, during the reaction sequences and synthetic schemes described herein, the order of certain steps may be changed, such as the introduction and removal of protecting groups. One of ordinary skill in the art will recognize that certain groups may require protection from the reaction conditions via the use of protecting groups.

Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment or prevention includes use of the compounds to provide such treatment or prevention as is described herein. It is to be further understood, unless otherwise stated, any description of a method of treatment or prevention includes use of the compounds to prepare a medicament to treat or prevent such condition. The treatment or prevention includes treatment or prevention of human or non-human animals including rodents and other disease models.

It is to be understood that, unless otherwise stated, any description of a method of treatment includes use of the compounds to provide such treatment as is described herein. It is to be further understood, unless otherwise stated, any description of a method of treatment includes use of the compounds to prepare a medicament to treat such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein.

A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990).

These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved.

One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount,” refers to an amount of a pharmaceutical agent to treat, ameliorate, and/or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans.

Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), cyclodextrins and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, capsules or sachets. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, transmucosal administration, detergents, bile salts, and fusidic acid derivatives. Transmucosal administration can be accomplished through the use of nasal sprays, powders or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure is dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage.

Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refers to derivatives of the compounds of the present disclosure wherein the parent compound is modified by making acid or base salts thereof. Examples of pharmaceutically acceptable salts include, but are not limited to, mineral organic acid salts of basic residues such as amines, alkali organic salts of acidic residues such as carboxylic acids, and the like. The pharmaceutically acceptable salts include the conventional non-toxic salts or the quaternary ammonium salts of the parent compound formed, for example, from non-toxic inorganic organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like.

The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As used herein, the phrase “compound of the disclosure” refers to those compounds which are disclosed herein, both generically and specifically.

### Compounds of the Present Disclosure

In some aspects, the present disclosure provides a compound of Formula (I):

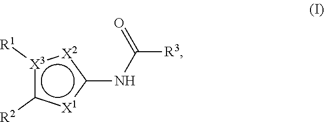

or a pharmaceutically acceptable salt, solvate, clathrate, hydrate, stereoisomer, or tautomer thereof, wherein:
X¹ is CH, S, or N;
X² is N, S, or O;
X³ is C or N;
R¹ and R², together with the atoms to which they are attached, form a C₆-C₁₀ aryl or 5- to 10-membered heteroaryl, wherein the C₆-C₁₀ aryl or 5- to 10-membered heteroaryl is optionally substituted with one or more R^{a};
each R^{a} independently is oxo, halo, cyano, —OH, —NH₂, —C(O)N(R^{b})(R^{c}), —N(R^{b})C(O)(R^{c}), C₁-C₆ alkyl, C₂-C₆ alkenyl, C₂-C₆ alkynyl, C₁-C₆ haloalkyl, C₃-C₁₀ cycloalkyl, C₄-C₁₀ cycloalkyl, C₆-C₁₀ aryl, 5- to 10-membered heteroaryl, 3- to 10-membered heterocycloalkyl, or 4- to 10-membered heterocycloalkenyl, wherein the C₁-C₆ alkyl, C₂-C₆ alkenyl, C₂-C₆ alkynyl, C₁-C₆ haloalkyl, C₃-C₁₀ cycloalkyl, C₄-C₁₀ cycloalkyl, C₆-C₁₀ aryl, 5- to 10-membered heteroaryl, 3- to 10-membered heterocycloalkyl, or 4- to 10-membered heterocycloalkenyl is optionally substituted with one or more R^{a1};
each R^{b} and R^{c} independently is H, C₁-C₆ alkyl, C₃-C₁₀ cycloalkyl, or 3- to 10-membered heterocycloalkyl, wherein the C₁-C₆ alkyl, C₃-C₁₀ cycloalkyl, or 3- to 10-membered heterocycloalkyl is optionally substituted with one or more —OH or C₁-C₆ haloalkyl; or
   R^{b} and R^{c}, together with the atoms to which they are attached, form a 3- to 10-membered heterocycloalkyl, wherein the 3- to 10-membered heterocycloalkyl is optionally substituted with one or more —OH, —O(C₁-C₆ haloalkyl), —O(C₁-C₆ alkyl), —C(O)(O—(C₁-C₆ alkyl)), C₁-C₆ alkyl, or —N(C₁-C₆ alkyl)₂;
each R^{a1} independently is oxo, halo, cyano, —OH, —O(C₃-C₁₀ cycloalkyl), —NH₂, —NH(C₁-C₆ alkyl), —N(C₁-C₆ alkyl)₂, —C(O)OH, —C(O)O(C₁-C₆ alkyl), C₁-C₆ alkyl, C₂-C₆ alkenyl, C₂-C₆ alkynyl, C₁-C₆ haloalkyl, C₁-C₆ alkoxy, C₁-C₆ haloalkoxy, —O—(C₃-C₁₀ cycloalkyl), C₃-C₁₀ cycloalkyl, 3- to 10-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl is optionally substituted with one or more R^{a2};
each R^{a2} independently is oxo, halo, cyano, —OH, or —NH₂;
R³ is C₆-C₁₀ aryl or 6- to 10-membered heteroaryl, wherein the C₆-C₁₀ aryl or 6- to 10-membered heteroaryl is substituted with one or more R^{3a};
each R^{3a} independently is halo, cyano, —OH, —NH₂, C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, —O(C₆-C₁₀ aryl), C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, —O(C₆-C₁₀ aryl), C₆-C₁₀ aryl, or 5- to 10-membered heteroaryl is optionally substituted with one or more R^{3a1}; and
each R^{3a1} independently is oxo, halo, cyano, —NH₂, —NH—C(O)O(C₁-C₆ alkyl), —C(O)(C₁-C₆ alkyl), —C(O)NH₂, —C(O)(O—(C₁-C₆ alkyl)), C₁-C₆ alkyl optionally substituted with one or more C₃-C₁₀ cycloalkyl, C₂-C₆ alkenyl, C₂-C₆ alkynyl, C₁-C₆ haloalkyl, C₁-C₆ alkoxy, —O(C₁-C₆ haloalkyl), C₃-C₁₀ cycloalkyl, C₆-C₁₀ aryl, or 5- to 10-membered heteroaryl;
provided that:
(i) when R^{3a} is halo and R¹ and R², together with the atoms to which they are attached, form a 6-membered heteroaryl then R^{a} is not —CF₃; or
(ii) X¹ is CH or N; and R¹ and R², together with the atoms to which they are attached, form a pyridine optionally substituted with one or more R^{a}; or
(iii) X² is N or O; and R¹ and R², together with the atoms to which they are attached, form a pyridine optionally substituted with one or more R^{a}; or
(iv) when R¹ and R², together with the atoms to which they are attached, form a pyridine optionally substituted with one or more R^{a}, then X¹ is not S; or
(v) when R¹ and R², together with the atoms to which they are attached, form a pyridine optionally substituted with one or more R^{a}, then X² is not S; or
(vi) when R¹ and R², together with the atoms to which they are attached, form a pyridine optionally substituted with one or more R^{a}, then

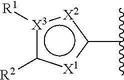

is not

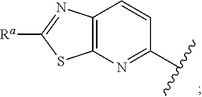

or
(vii) when R^{a} is

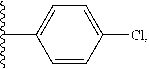 [SMILES: *c1ccc(Cl)cc1]

then R³ is not

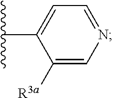

or
(viii) when R^{a} is

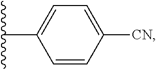 [SMILES: *c1ccc(C#N)cc1]

then R³ is not

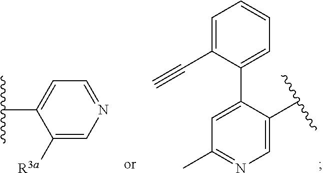

or
(ix) when R¹ and R², together with the atoms to which they are attached, form a pyrazine substituted with one or more R^{a}, and R^{a} is phenyl substituted with one or more R^{a1}, then R^{a1} is not —Cl, —F, —CN, —O(CH₃), —O(CF₃), —O(CHF₂), —CH₃, or cyclopropyl; or
(x) when R¹ and R², together with the atoms to which they are attached, form a pyrazine substituted with one or more R^{a}, and R^{a} is pyridine substituted with one or more R^{a1}, then R^{a1} is not —Cl, —CN, —CF₃, —O(CH₃), —O(CHF₂), cyclopropyl, —C(CH₃)₂(CN), or —C(CH₃)₂(OH); or
(xi) when R¹ and R², together with the atoms to which they are attached, form a pyrazine substituted with one or more R^{a}, then R^{a} is not

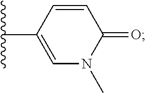 [SMILES: *c1ccc(=O)n(C)c1]

or
(xii) the compound of Formula (I) is not 5′-methoxy-2′,6-dimethyl-N-(6-(tetrahydrofuran-3-yl)thiazolo[4,5-b]pyrazin-2-yl)-[4,4′-bipyridine]-3-carboxamide.

In some aspects, the present disclosure provides a compound of Formula (I):

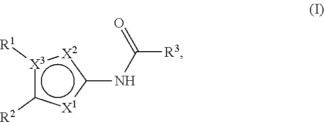

or a pharmaceutically acceptable salt, solvate, clathrate, hydrate, stereoisomer, or tautomer thereof, wherein:
X¹ is CH, S, or N;
X² is N, S, or O;
X³ is C or N;
R¹ and R², together with the atoms to which they are attached, form a C₆-C₁₀ aryl or 5- to 10-membered heteroaryl, wherein the C₆-C₁₀ aryl or 5- to 10-membered heteroaryl is optionally substituted with one or more R^{a};
each R^{a} independently is oxo, halo, cyano, —OH, —NH₂, —C(O)N(R^{b})(R^{c}, —N(R^{b})C(O)(R^{c}), C₁-C₆ alkyl, C₂-C₆ alkenyl, C₂-C₆ alkynyl, C₁-C₆ haloalkyl, C₃-C₁₀ cycloalkyl, C₆-C₁₀ aryl, 5- to 10-membered heteroaryl, or 3- to 10-membered heterocycloalkyl, wherein the C₁-C₆ alkyl, C₂-C₆ alkenyl, C₂-C₆ alkynyl, C₁-C₆ haloalkyl, C₃-C₁₀ cycloalkyl, C₆-C₁₀ aryl, 5- to 10-membered heteroaryl, or 3- to 10-membered heterocycloalkyl is optionally substituted with one or more R^{a1}.
each R^{b} and R^{c} independently is H, C₁-C₆ alkyl, C₃-C₁₀ cycloalkyl, or 3- to 10-membered heterocycloalkyl, wherein the C₁-C₆ alkyl, C₃-C₁₀ cycloalkyl, or 3- to 10-membered heterocycloalkyl is optionally substituted with one or more —OH or C₁-C₆ haloalkyl; or
R^{b} and R^{c}, together with the atoms to which they are attached, form a 3- to 10-membered heterocycloalkyl, wherein the 3- to 10-membered heterocycloalkyl is optionally substituted with one or more —OH, —O(C₁-C₆ haloalkyl), —O(C₁-C₆ alkyl), —C(O)(O—(C₁-C₆ alkyl)), C₁-C₆ alkyl, or —N(C₁-C₆ alkyl)₂;
each R^{a1} independently is oxo, halo, cyano, —OH, —O(C₃-C₁₀ cycloalkyl), —NH₂, —NH(C₁-C₆ alkyl), —N(C₁-C₆ alkyl)₂, —C(O)OH, —C(O)O(C₁-C₆ alkyl), C₁-C₆ alkyl, C₂-C₆ alkenyl, C₂-C₆ alkynyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl is optionally substituted with one or more R^{a2};
each R^{a2} independently is oxo, halo, cyano, —OH, or —NH₂;
R³ is C₆-C₁₀ aryl or 6- to 10-membered heteroaryl, wherein the C₆-C₁₀ aryl or 6- to 10-membered heteroaryl is substituted with one or more R^{3a};
each R^{3a} independently is halo, cyano, —OH, —NH₂, C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, C₆-C₁₀ aryl, or 5- to 10-membered heteroaryl is optionally substituted with one or more R^{3a1}; and
each R^{3a1} independently is oxo, halo, cyano, —C(O)(C₁-C₆ alkyl), —C(O)NH₂, —C(O)(0-(C₁-C₆ alkyl)), C₁-C₆ alkyl optionally substituted with one or more C₃-C₁₀ cycloalkyl, C₂-C₆ alkenyl, C₂-C₆ alkynyl, C₁-C₆ haloalkyl, C₁-C₆ alkoxy, —O(C₁-C₆ haloalkyl), C₃-C₁₀ cycloalkyl, C₆-C₁₀ aryl, or 5- to 10-membered heteroaryl;
   wherein, when R^{3a} is halo and R¹ and R², together with the atoms to which they are attached, form a 6-membered heteroaryl then R^{a} is not —CF₃.

It is understood that, for a compound of the present disclosure, variables X¹, X², X³, R¹, R², R^{a}, R^{b}, R^{c}, R^{a1}, R^{a2}, R³, R^{3a}, and R^{3a1} can each be, where applicable, selected from the groups described herein, and any group described herein for any of variables X¹, X², X³, R¹, R², R^{a}, R^{b}, R^{c}, R^{a1}, R^{a2}, R³, R^{3a}, and R^{3a1} can be combined, where applicable, with any group described herein for one or more of the remainder of variables X¹, X², X³, R¹, R², R^{a}, R^{b}, R^{c}, R^{a1}, R^{a2}, R³, R^{3a}, and R^{3a1}

In some embodiments, X¹ is CH or S.

In some embodiments, X¹ is S or N.

In some embodiments, X¹ is CH or N.

In some embodiments, X¹ is CH. In some embodiments, X¹ is S. In some embodiments, X¹ is N.

In some embodiments, X² is N or S.

In some embodiments, X² is N or O.

In some embodiments, X² is O or S.

In some embodiments, X² is N. In some embodiments, X² is S. In some embodiments, X² is O.

In some embodiments, X³ is C. In some embodiments, X³ is N.

In some embodiments, R¹ and R², together with the atoms to which they are attached, form a C₆ aryl or 6-membered heteroaryl.

In some embodiments, R¹ and R², together with the atoms to which they are attached, form a C₆ aryl or 6-membered heteroaryl, wherein the aryl or heteroaryl is optionally substituted with one or more R^{a}.

In some embodiments, R¹ and R², together with the atoms to which they are attached, form a C₆ aryl or 6-membered heteroaryl, wherein the aryl or heteroaryl is substituted with one or more R^{a}.

In some embodiments, R¹ and R², together with the atoms to which they are attached, form a C₆-C₁₀ aryl.

In some embodiments, R¹ and R², together with the atoms to which they are attached, form a C₆-C₁₀ aryl optionally substituted with one or more R^{a}

In some embodiments, R¹ and R², together with the atoms to which they are attached, form a C₆-C₁₀ aryl substituted with one or more R^{a}

In some embodiments, R¹ and R², together with the atoms to which they are attached, form a C₆ aryl.

In some embodiments, R¹ and R², together with the atoms to which they are attached, form a C₆ aryl optionally substituted with one or more R^{a}.

In some embodiments, R¹ and R², together with the atoms to which they are attached, form a C₆ aryl substituted with one or more R^{a}

In some embodiments, R¹ and R², together with the atoms to which they are attached, form a 5- to 10-membered heteroaryl.

In some embodiments, R¹ and R², together with the atoms to which they are attached, form a 5- to 10-membered heteroaryl optionally substituted with one or more R^{a}

In some embodiments, R¹ and R², together with the atoms to which they are attached, form a 5- to 10-membered heteroaryl substituted with one or more R^{a}.

In some embodiments, R¹ and R², together with the atoms to which they are attached, form a 5-membered heteroaryl.

In some embodiments, R¹ and R², together with the atoms to which they are attached, form a 5-membered heteroaryl optionally substituted with one or more R^{a}

In some embodiments, R¹ and R², together with the atoms to which they are attached, form a 5-membered heteroaryl substituted with one or more R^{a}

In some embodiments, R¹ and R², together with the atoms to which they are attached, form a 6-membered heteroaryl.

In some embodiments, R¹ and R², together with the atoms to which they are attached, form a 6-membered heteroaryl optionally substituted with one or more R^{a}.

In some embodiments, R¹ and R², together with the atoms to which they are attached, form a 6-membered heteroaryl substituted with one or more R^{a}

In some embodiments, R¹ and R², together with the atoms to which they are attached, form pyrazinyl, pyridinyl, or diazolyl.

In some embodiments, R¹ and R², together with the atoms to which they are attached, form pyrazinyl, pyridinyl, or diazolyl, wherein the pyrazinyl, pyridinyl, or diazolyl is optionally substituted with one or more R^{a}.

In some embodiments, R¹ and R², together with the atoms to which they are attached, form pyrazinyl, pyridinyl, or diazolyl, wherein the pyrazinyl, pyridinyl, or diazolyl is substituted with one or more R^{a}.

In some embodiments, R¹ and R², together with the atoms to which they are attached form pyrazinyl.

In some embodiments, R¹ and R², together with the atoms to which they are attached form pyrazinyl optionally substituted with one or more R^{a}.

In some embodiments, R¹ and R², together with the atoms to which they are attached form pyrazinyl substituted with one or more R^{a}

In some embodiments, R¹ and R², together with the atoms to which they are attached form pyridinyl.

In some embodiments, R¹ and R², together with the atoms to which they are attached form pyridinyl optionally substituted with one or more R^{a}.

In some embodiments, R¹ and R², together with the atoms to which they are attached form pyridinyl substituted with one or more R^{a}

In some embodiments, R¹ and R², together with the atoms to which they are attached form diazolyl.

In some embodiments, R¹ and R², together with the atoms to which they are attached form diazolyl optionally substituted with one or more R^{a}.

In some embodiments, R¹ and R², together with the atoms to which they are attached form diazolyl substituted with one or more R^{a}.

In some embodiments, each R^{a} independently is oxo, halo, —C(O)N(R^{b})(R^{c}), —N(R^{b})C(O)(R^{c}), C₁-C₆ alkyl, C₁-C₆ haloalkyl, C₃-C₁₀ cycloalkyl, C₄-C₁₀ cycloalkenyl, C₆-C₁₀ aryl, 5- to 10-membered heteroaryl, 3- to 10-membered heterocycloalkyl, or 3- to 10-membered heterocycloalkenyl.

In some embodiments, each R^{a} independently is oxo, halo, —C(O)N(R^{b})(R^{c}), —N(R^{b})C(O)(R^{c}), C₁-C₆ alkyl, C₁-C₆ haloalkyl, C₃-C₁₀ cycloalkyl, C₄-C₁₀ cycloalkenyl, C₆-C₁₀ aryl, 5- to 10-membered heteroaryl, 3- to 10-membered heterocycloalkyl, or 3- to 10-membered heterocycloalkenyl, wherein the C₁-C₆ alkyl, C₁-C₆ haloalkyl, C₃-C₁₀ cycloalkyl, C₄-C₁₀ cycloalkenyl, C₆-C₁₀ aryl, 5- to 10-membered heteroaryl, 3- to 10-membered heterocycloalkyl, or 3- to 10-membered heterocycloalkenyl is optionally substituted with one or more R^{a1}.

In some embodiments, each R^{a} independently is oxo, halo, —C(O)N(R^{b})(R^{c}), —N(R^{b})C(O)(R^{c}), C₁-C₆ alkyl, C₁-C₆ haloalkyl, C₃-C₁₀ cycloalkyl, C₆-C₁₀ aryl, 5- to 10-membered heteroaryl, or 3- to 10-membered heterocycloalkyl.

In some embodiments, each R^{a} independently is oxo, halo, —C(O)N(R^{b})(R^{c}), —N(R^{b})C(O)(R^{c}), C₁-C₆ alkyl, C₁-C₆ haloalkyl, C₃-C₁₀ cycloalkyl, C₆-C₁₀ aryl, 5- to 10-membered heteroaryl, or 3- to 10-membered heterocycloalkyl, wherein the C₁-C₆ alkyl, C₁-C₆ haloalkyl, C₃-C₁₀ cycloalkyl, C₆-C₁₀ aryl, 5- to 10-membered heteroaryl, or 3- to 10-membered heterocycloalkyl is optionally substituted with one or more R^{a1}.

In some embodiments, each R^{a} independently is oxo, halo, —C(O)N(R^{b})(R^{c}), —N(R^{b})C(O)(R^{c}), C₁-C₆ alkyl, C₁-C₆ haloalkyl, C₃-C₁₀ cycloalkyl, C₆-C₁₀ aryl, 5- to 10-membered heteroaryl, or 3- to 10-membered heterocycloalkyl, wherein the C₁-C₆ alkyl, C₁-C₆ haloalkyl, C₃-C₁₀ cycloalkyl, C₆-C₁₀ aryl, 5- to 10-membered heteroaryl, or 3- to 10-membered heterocycloalkyl is substituted with one or more R^{a1}.

In some embodiments, each R^{a} independently is oxo.

In some embodiments, each R^{a} independently is halo.

In some embodiments, each R^{a} independently is F, Cl, Br, or I. In some embodiments, each R^{a} independently is F, Cl, or Br. In some embodiments, each R^{a} independently is F or Cl.

In some embodiments, each R^{a} independently is F. In some embodiments, each R^{a} independently is Cl. In some embodiments, each R^{a} independently is Br. In some embodiments, each R^{a} independently is I.

In some embodiments, each R^{a} independently is cyano.

In some embodiments, each R^{a} independently is —OH.

In some embodiments, each R^{a} independently is —NH₂.

In some embodiments, each R^{a} independently is —C(O)N(R^{b})(R^{c}).

In some embodiments, each R^{a} independently is —N(R^{b})C(O)(R^{c}).

In some embodiments, each R^{a} independently is C₁-C₆ alkyl.

In some embodiments, each R^{a} independently is C₁-C₆ alkyl optionally substituted with one or more R^{a1}.

In some embodiments, each R^{a} independently is C₁-C₆ alkyl substituted with one or more R^{a1}.

In some embodiments, each R^{a} independently is methyl. In some embodiments, each R^{a} independently is ethyl. In some embodiments, each R^{a} independently is propyl. In some embodiments, each R^{a} independently is butyl. In some embodiments, each R^{a} independently is pentyl. In some embodiments, each R^{a} independently is hexyl. In some embodiments, each R^{a} independently is isopropyl. In some embodiments, each R^{a} independently is isobutyl. In some embodiments, each R^{a} independently is isopentyl. In some embodiments, each R^{a} independently is isohexyl. In some embodiments, each R^{a} independently is secbutyl. In some embodiments, each R^{a} independently is secpentyl. In some embodiments, each R^{a} independently is sechexyl. In some embodiments, each R^{a} independently is tertbutyl.

In some embodiments, each R^{a} independently is C₂-C₆ alkenyl (e.g., ethenyl, propenyl, butenyl).

In some embodiments, each R^{a} independently is C₂-C₆ alkenyl (e.g., ethenyl, propenyl, butenyl) optionally substituted with one or more R^{a1}.

In some embodiments, each R^{a} independently is C₂-C₆ alkenyl (e.g., ethenyl, propenyl, butenyl) substituted with one or more R^{a1}.

In some embodiments, each R^{a} independently is C₂-C₆ alkynyl (e.g., ethynyl, propynyl, butynyl).

In some embodiments, each R^{a} independently is C₂-C₆ alkynyl (e.g., ethynyl, propynyl, butynyl) optionally substituted with one or more R^{a1}.

In some embodiments, each R^{a} independently is C₂-C₆ alkynyl (e.g., ethynyl, propynyl, butynyl) substituted with one or more R^{a1}.

In some embodiments, each R^{a} independently is C₁-C₆ haloalkyl.

In some embodiments, each R^{a} independently is C₁-C₆ haloalkyl optionally substituted with one or more R^{a1}.

In some embodiments, each R^{a} independently is C₁-C₆ haloalkyl substituted with one or more R^{a1}.

In some embodiments, each R^{a} independently is halomethyl. In some embodiments, each R^{a} independently is haloethyl. In some embodiments, each R^{a} independently is halopropyl. In some embodiments, each R^{a} independently is halobutyl. In some embodiments, each R^{a} independently is halopentyl. In some embodiments, each R^{a} independently is halohexyl.

In some embodiments, each R^{a} independently is C₃-C₁₀ cycloalkyl.

In some embodiments, each R^{a} independently is C₃-C₁₀ cycloalkyl optionally substituted with one or more R^{a1}.

In some embodiments, each R^{a} independently is C₃-C₁₀ cycloalkyl substituted with one or more R^{a1}.

In some embodiments, each R^{a} independently is C₃-C₇ cycloalkyl.

In some embodiments, each R^{a} independently is C₃-C₇ cycloalkyl optionally substituted with one or more R^{a1}.

In some embodiments, each R^{a} independently is C₃-C₇ cycloalkyl substituted with one or more R^{a1}.

In some embodiments, each R^{a} independently is C₄-C₁₀ cycloalkenyl.

In some embodiments, each R^{a} independently is C₄-C₁₀ cycloalkenyl optionally substituted with one or more R^{a1}.

In some embodiments, each R^{a} independently is C₄-C₁₀ cycloalkenyl substituted with one or more R^{a1}.

In some embodiments, each R^{a} independently is C₆-C₁₀ aryl.

In some embodiments, each R^{a} independently is C₆-C₁₀ aryl optionally substituted with one or more R^{a1}.

In some embodiments, each R^{a} independently is C₆-C₁₀ aryl substituted with one or more R^{a1}.

In some embodiments, each R^{a} independently is C₆ aryl.

In some embodiments, each R^{a} independently is C₆ aryl optionally substituted with one or more R^{a1}.

In some embodiments, each R^{a} independently is C₆ aryl substituted with one or more R^{a1}.

In some embodiments, each R^{a} independently is 5- to 10-membered heteroaryl.

In some embodiments, each R^{a} independently is 5- to 10-membered heteroaryl optionally substituted with one or more R^{a1}.

In some embodiments, each R^{a} independently is 5- to 10-membered heteroaryl substituted with one or more R^{a1}.

In some embodiments, each R^{a} independently is 5- or 6-membered heteroaryl.

In some embodiments, each R^{a} independently is 5- or 6-membered heteroaryl optionally substituted with one or more R^{a1}

In some embodiments, each R^{a} independently is 5- or 6-membered heteroaryl substituted with one or more R^{a1}.

In some embodiments, each R^{a} independently is 3- to 10-membered heterocycloalkyl.

In some embodiments, each R^{a} independently is 3- to 10-membered heterocycloalkyl optionally substituted with one or more R^{a1}.

In some embodiments, each R^{a} independently is 3- to 10-membered heterocycloalkyl substituted with one or more R^{a1}.

In some embodiments, each R^{a} independently is 4- to 10-membered heterocycloalkenyl.

In some embodiments, each R^{a} independently is 4- to 10-membered heterocycloalkenyl optionally substituted with one or more R^{a1}.

In some embodiments, each R^{a} independently is 4- to 10-membered heterocycloalkenyl substituted with one or more R^{a1}.

In some embodiments, each R^{a} independently is C₃-C₁₀ cycloalkyl or 3- to 10-membered heterocycloalkyl.

In some embodiments, each R^{a} independently is C₃-C₁₀ cycloalkyl or 3- to 10-membered heterocycloalkyl, wherein the C₃-C₁₀ cycloalkyl or 3- to 10-membered heterocycloalkyl is optionally substituted with one or more R^{a1}.

In some embodiments, each R^{a} independently is C₃-C₁₀ cycloalkyl or 3- to 10-membered heterocycloalkyl, wherein the C₃-C₁₀ cycloalkyl or 3- to 10-membered heterocycloalkyl is substituted with one or more R^{a1}.

In some embodiments, each R^{a} independently is: —Cl, —Br, —CH₃, —CF₃,

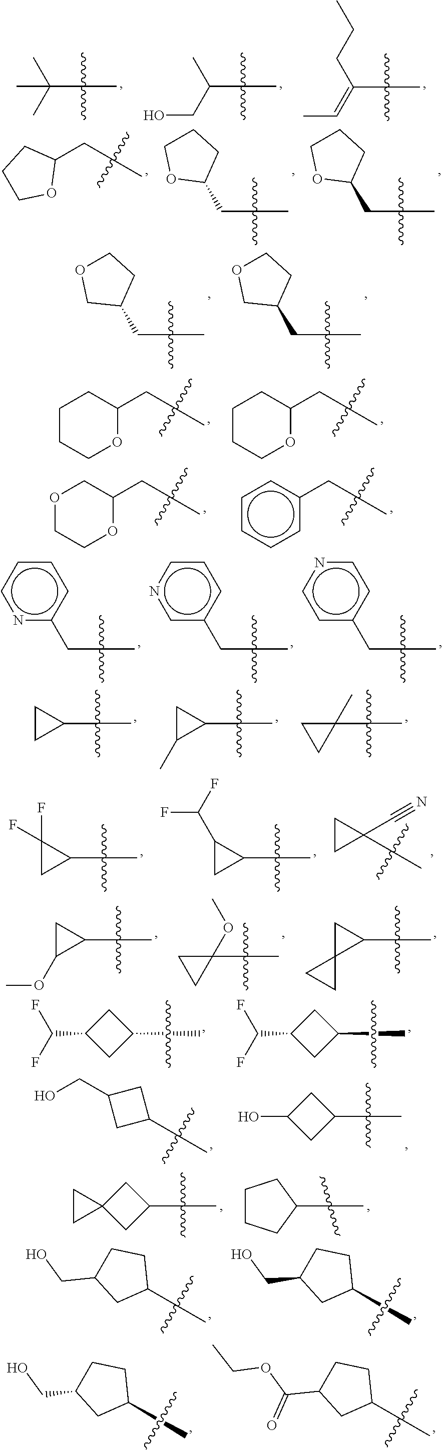

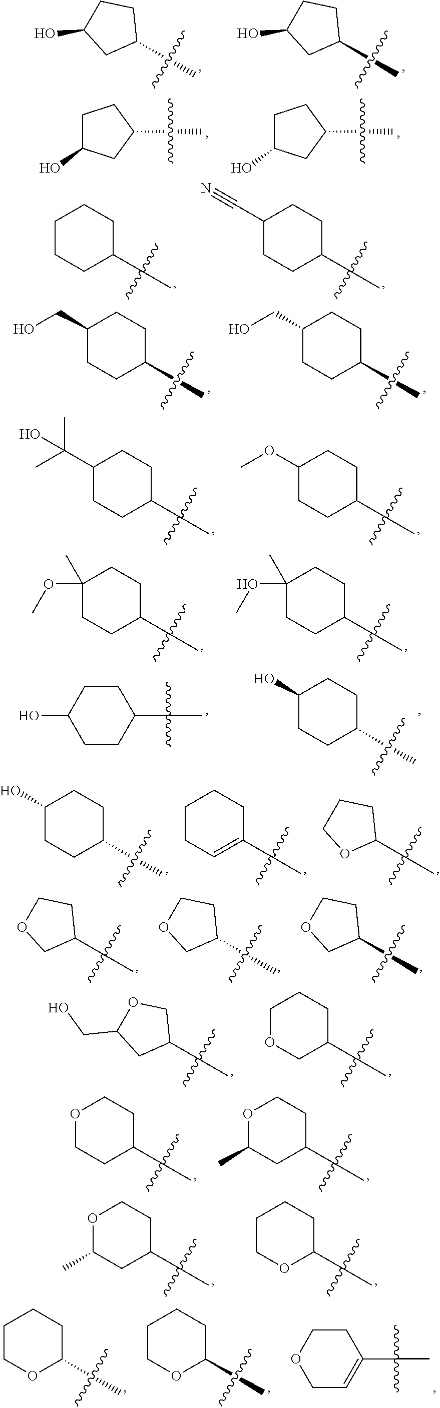

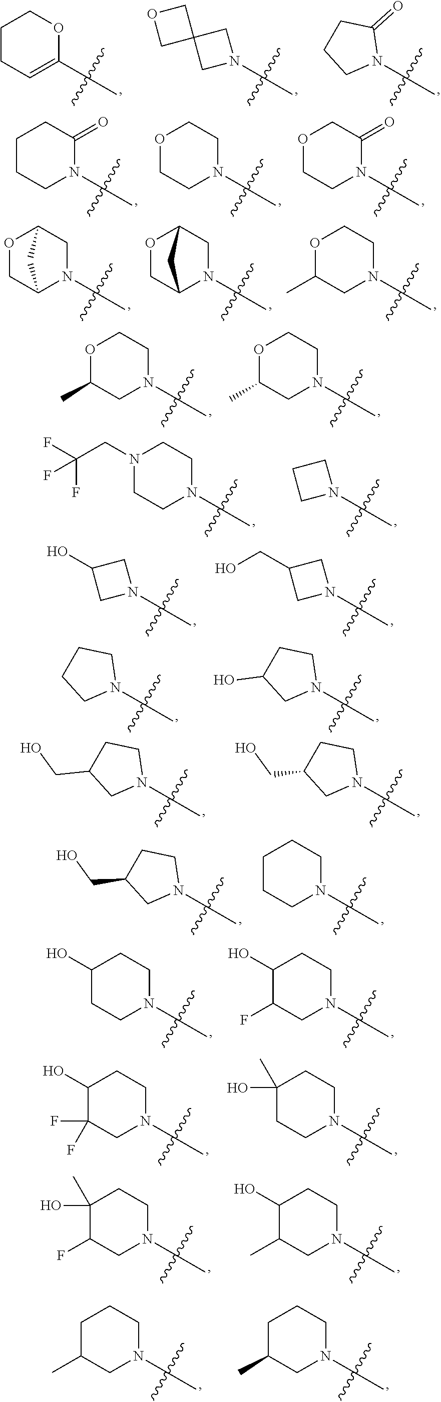

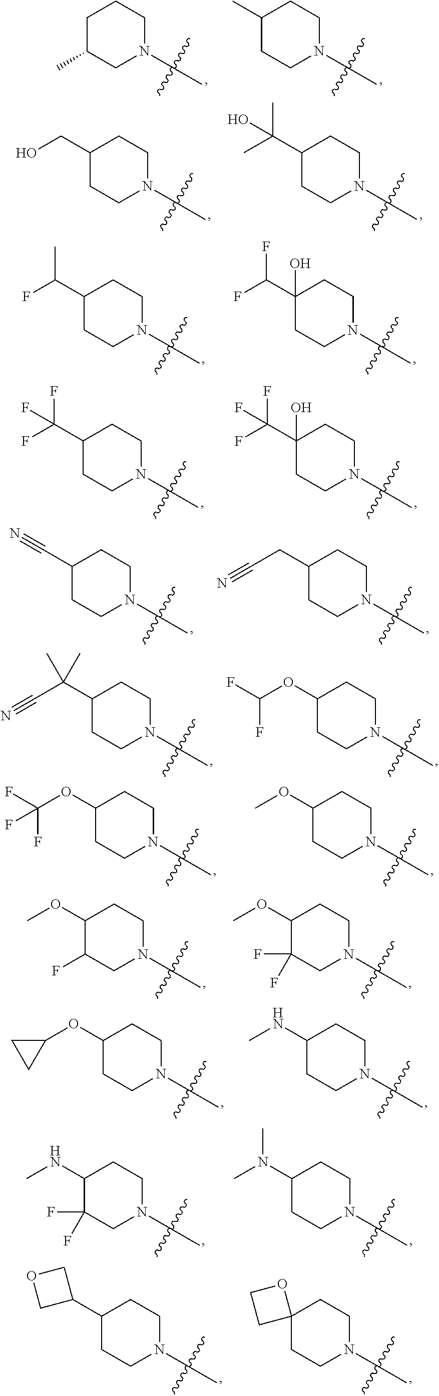

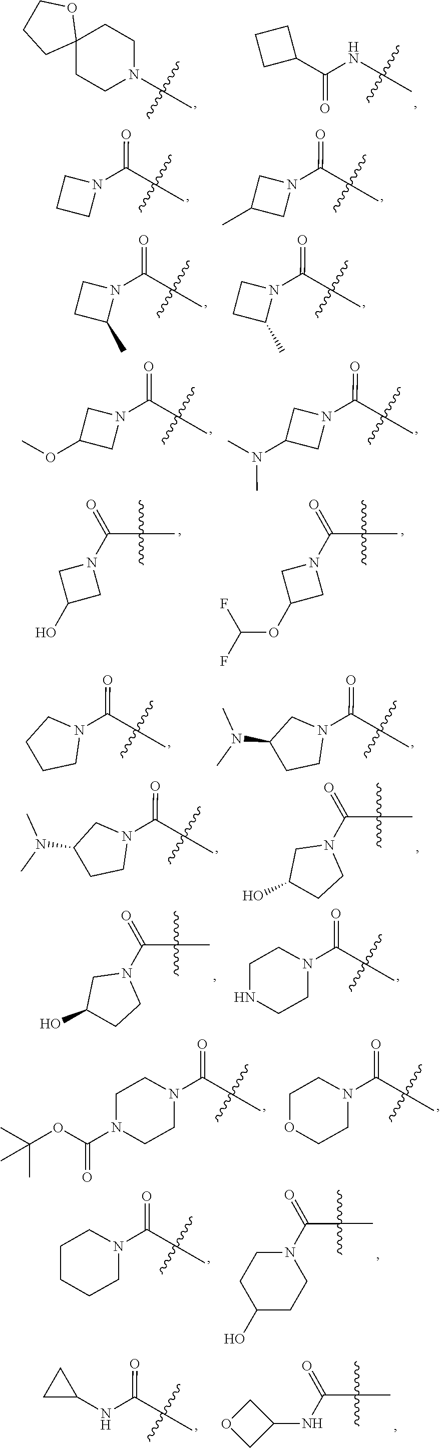

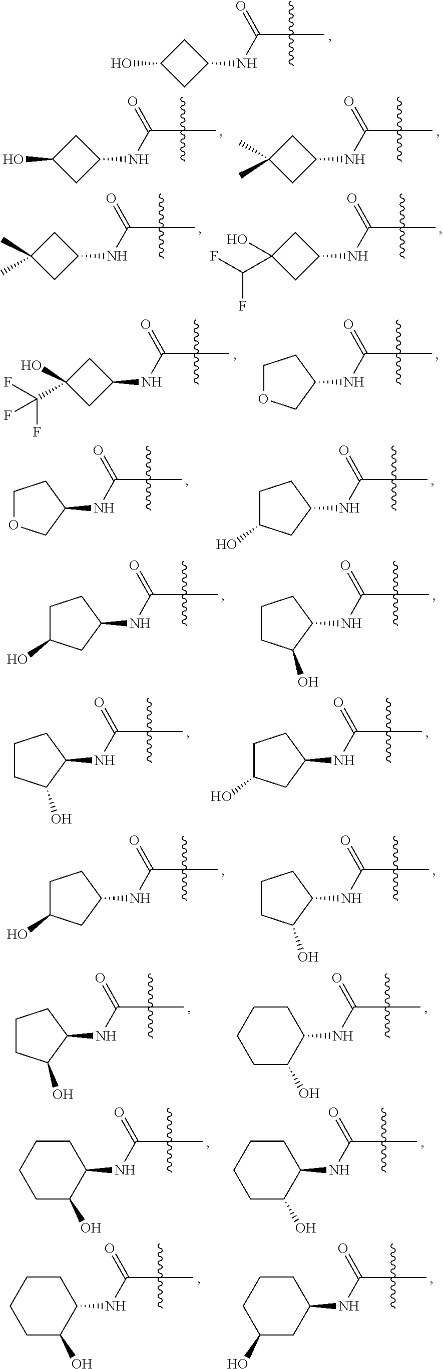

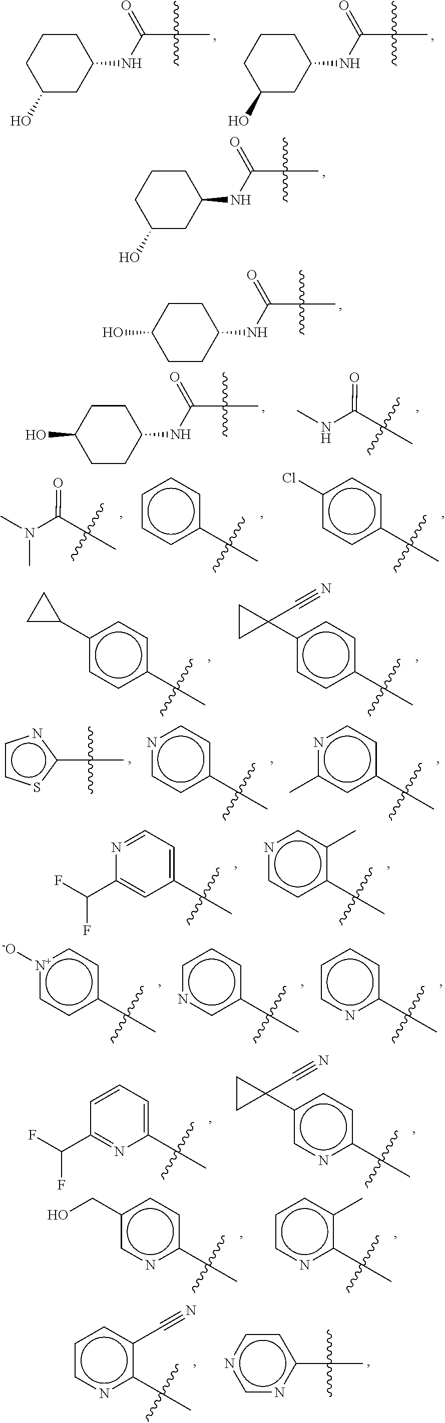

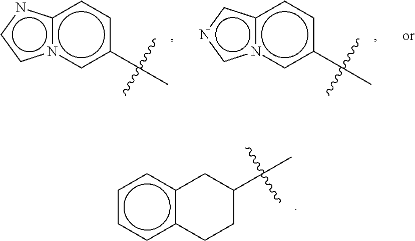

In some embodiments, each W independently is: oxo, —Cl, —Br, —CH₃, —CF₃,

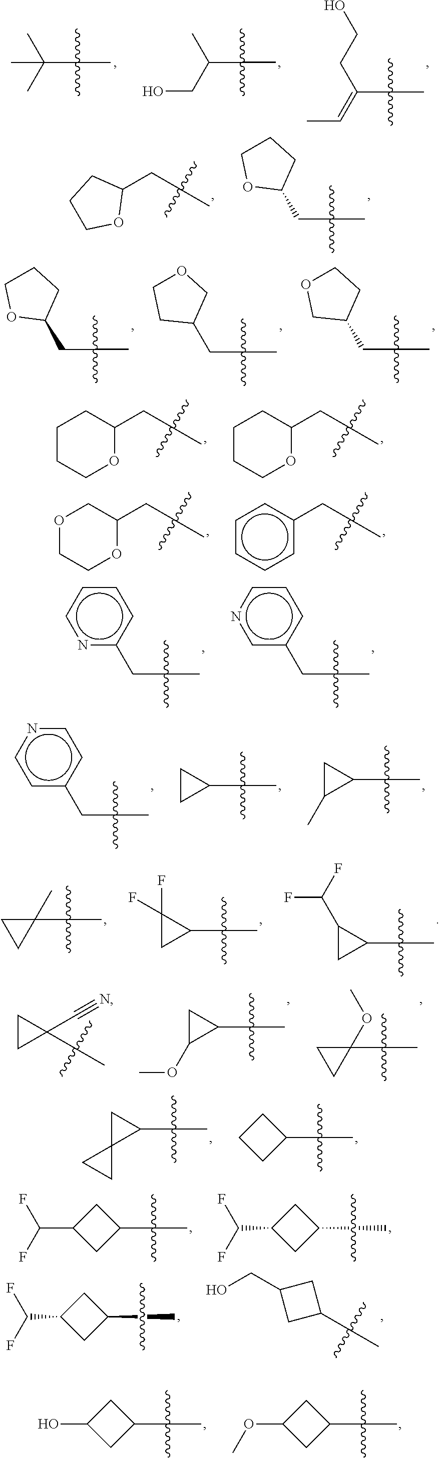

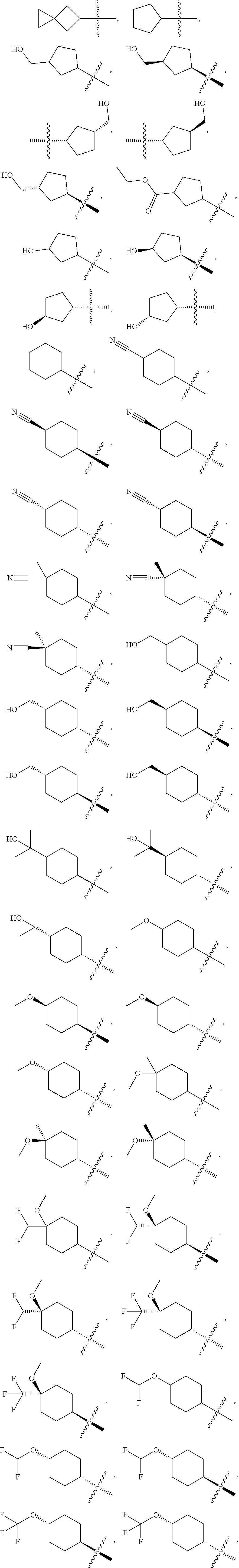

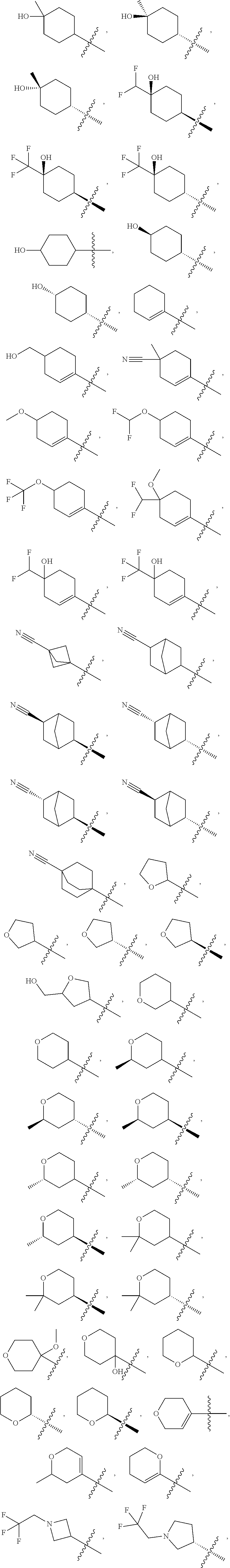

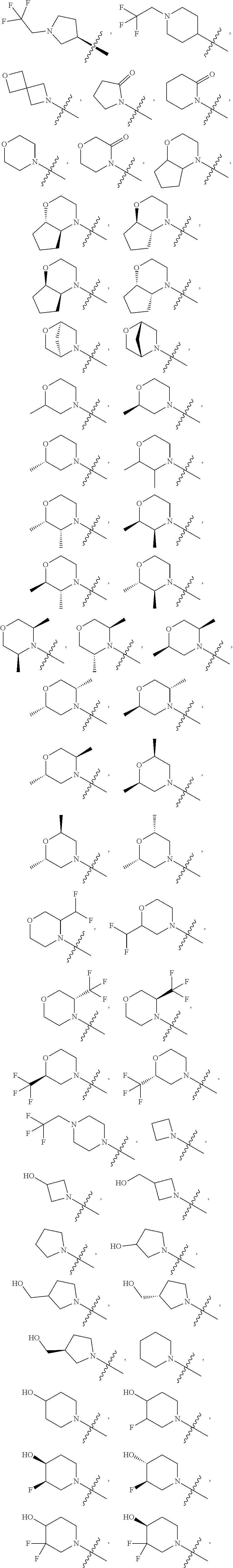

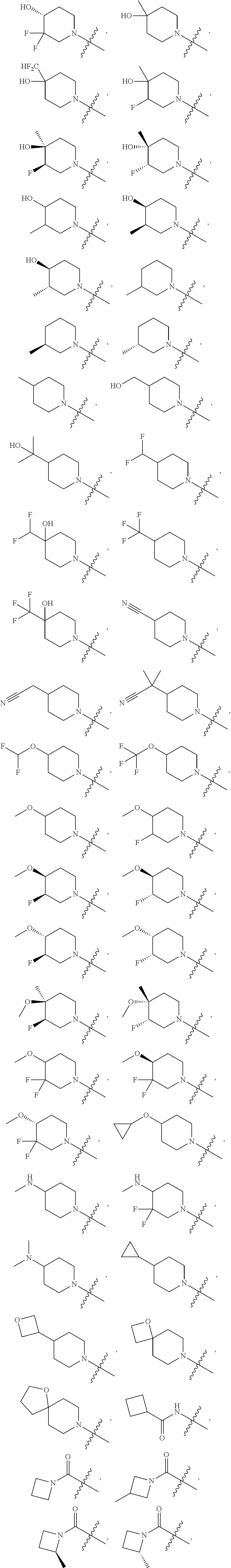

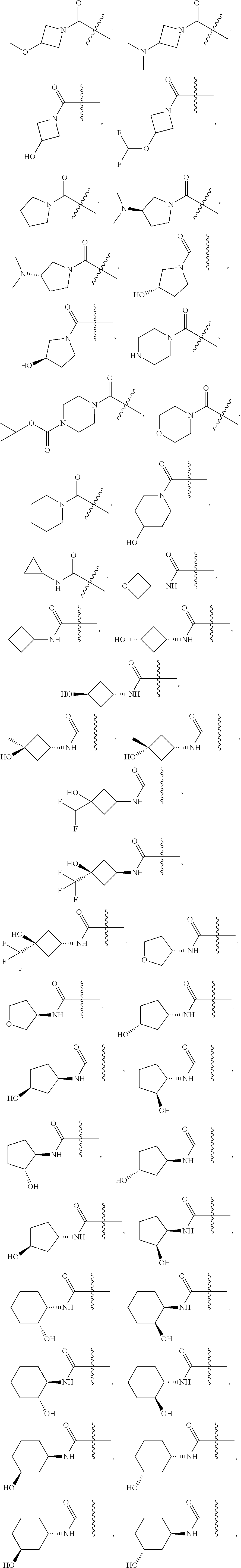

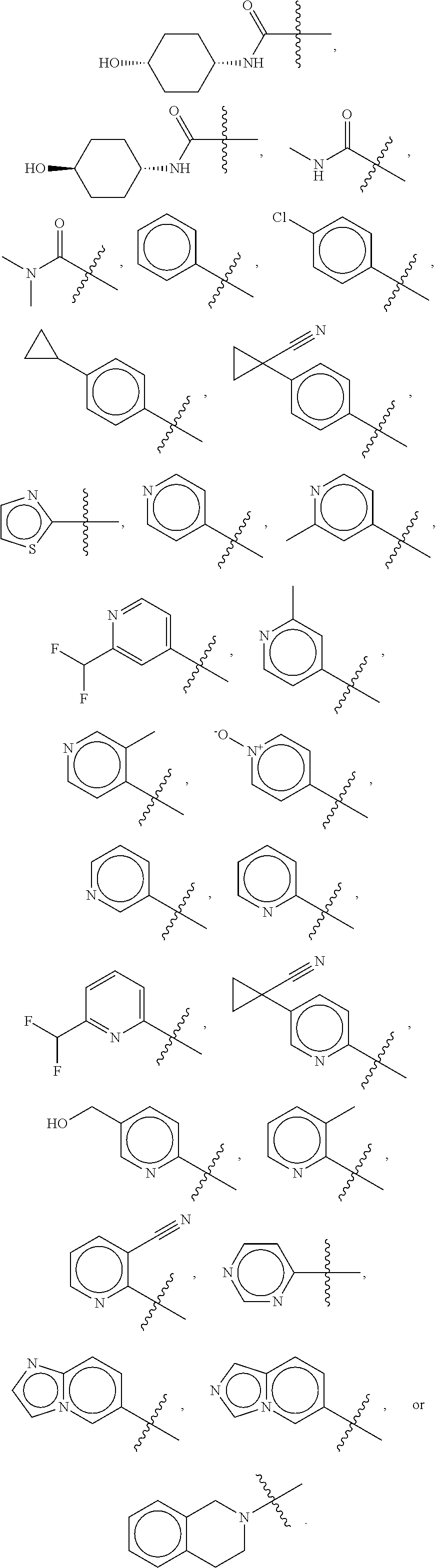

In some embodiments, each R^{b} and R^{c} independently is H, C₁-C₆ alkyl, C₃-C₁₀ cycloalkyl, or 3- to 10-membered heterocycloalkyl.

In some embodiments, each R^{b} and R^{c} independently is H, C₁-C₆ alkyl, C₃-C₁₀ cycloalkyl, or 3- to 10-membered heterocycloalkyl, wherein the C₁-C₆ alkyl, C₃-C₁₀ cycloalkyl, or 3- to 10-membered heterocycloalkyl is optionally substituted with one or more —OH or C₁-C₆ haloalkyl.

In some embodiments, each R^{b} and R^{c} independently is H, C₁-C₆ alkyl, C₃-C₁₀ cycloalkyl, or 3- to 10-membered heterocycloalkyl, wherein the C₁-C₆ alkyl, C₃-C₁₀ cycloalkyl, or 3- to 10-membered heterocycloalkyl is substituted with one or more —OH or C₁-C₆ haloalkyl.

In some embodiments, each R^{b} and R^{c} independently is H.

In some embodiments, each R^{b} and R^{c} independently is C₁-C₆ alkyl.

In some embodiments, each R^{b} and R^{c} independently is C₁-C₆ alkyl optionally substituted with —OH or C₁-C₆ haloalkyl.

In some embodiments, each R^{b} and R^{c} independently is C₁-C₆ alkyl substituted with —OH or C₁-C₆ haloalkyl.

In some embodiments, each R^{b} and R^{c} independently is methyl. In some embodiments, each R^{b} and R^{c} independently is ethyl. In some embodiments, each R^{b} and R^{c} independently is propyl. In some embodiments, each R^{b} and R^{c} independently is butyl. In some embodiments, each R^{b} and R^{c} independently is pentyl. In some embodiments, each R^{b} and R^{c} independently is hexyl. In some embodiments, each R^{b} and R^{c} independently is isopropyl. In some embodiments, each R^{b} and R^{c} independently is isobutyl. In some embodiments, each R^{b} and R independently is isopentyl. In some embodiments, each R^{b} and R^{c} independently is isohexyl.

In some embodiments, each R^{b} and R^{c} independently is secbutyl. In some embodiments, each R^{b} and R^{c} independently is secpentyl. In some embodiments, each R^{b} and R^{c} independently is sechexyl. In some embodiments, each R^{b} and R^{c} independently is tertbutyl.

In some embodiments, each R^{b} and R^{c} independently is C₃-C₁₀ cycloalkyl.

In some embodiments, each R^{b} and R^{c} independently is C₃-C₁₀ cycloalkyl optionally substituted with —OH or C₁-C₆ haloalkyl.

In some embodiments, each R^{b} and R^{c} independently is C₃-C₁₀ cycloalkyl substituted with —OH or C₁-C₆ haloalkyl.

In some embodiments, each R^{b} and R^{c} independently is 3- to 10-membered heterocycloalkyl.

In some embodiments, each R^{b} and R^{c} independently is 3- to 10-membered heterocycloalkyl optionally substituted with —OH or C₁-C₆ haloalkyl.

In some embodiments, each R^{b} and R^{c} independently is 3- to 10-membered heterocycloalkyl substituted with —OH or C₁-C₆ haloalkyl.

In some embodiments, R^{b} and R^{c}, together with the atoms to which they are attached, form a 3- to 10-membered heterocycloalkyl.

In some embodiments, R^{b} and R^{c}, together with the atoms to which they are attached, form a 3- to 10-membered heterocycloalkyl optionally substituted with one or more —OH, —O(C₁-C₆haloalkyl), C₁-C₆ alkyl, or —N(C₁-C₆ alkyl)₂.

In some embodiments, R^{b} and R^{c}, together with the atoms to which they are attached, form a 3- to 10-membered heterocycloalkyl substituted with one or more —OH, —O(C₁-C₆ haloalkyl), C₁-C₆ alkyl, or —N(C₁-C₆ alkyl)₂.

In some embodiments, each R^{b} and R^{c} independently is H, methyl, ethyl, cyclobutyl, cyclopropyl,

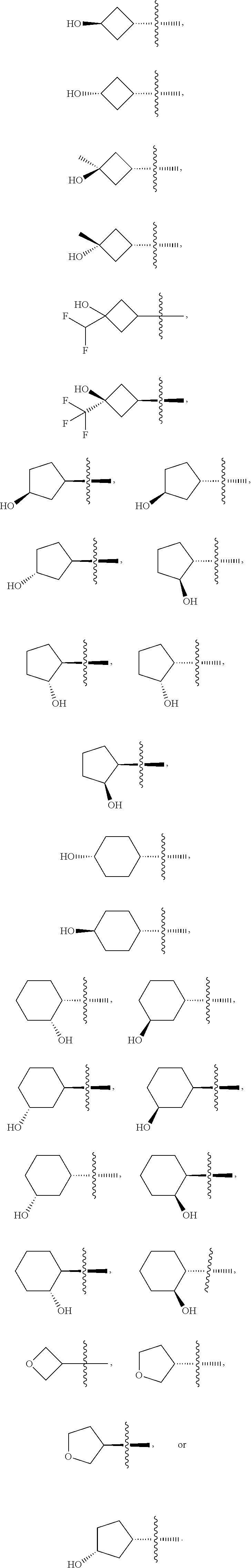

In some embodiments, R^{b} and R^{c}, together with the atoms to which they are attached, form a heterocycloalkyl selected from:

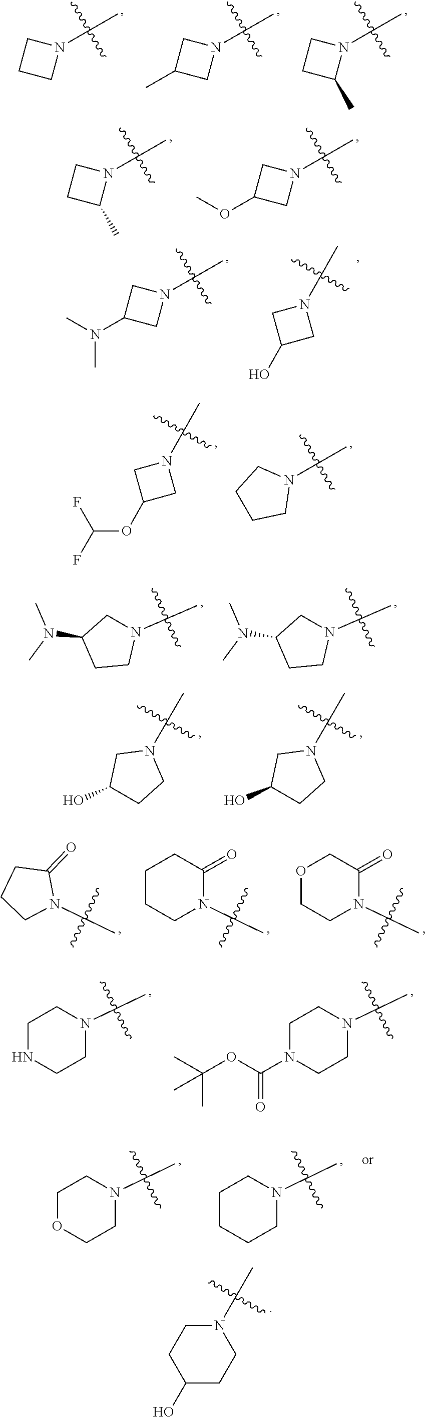

In some embodiments, R^{b} is H, C₁-C₆ alkyl, C₃-C₁₀ cycloalkyl, or 3- to 10-membered heterocycloalkyl.

In some embodiments, R^{b} is H, C₁-C₆ alkyl, C₃-C₁₀ cycloalkyl, or 3- to 10-membered heterocycloalkyl, wherein the C₁-C₆ alkyl, C₃-C₁₀ cycloalkyl, or 3- to 10-membered heterocycloalkyl is optionally substituted with one or more —OH or C₁-C₆ haloalkyl.

In some embodiments, R^{b} is H, C₁-C₆ alkyl, C₃-C₁₀ cycloalkyl, or 3- to 10-membered heterocycloalkyl, wherein the C₁-C₆ alkyl, C₃-C₁₀ cycloalkyl, or 3- to 10-membered heterocycloalkyl is substituted with one or more —OH or C₁-C₆ haloalkyl.

In some embodiments, R^{b} is H.

In some embodiments, R^{b} is C₁-C₆ alkyl.

In some embodiments, R^{b} is C₁-C₆ alkyl optionally substituted with —OH or C₁-C₆ haloalkyl.

In some embodiments, R^{b} is C₁-C₆ alkyl substituted with —OH or C₁-C₆haloalkyl.

In some embodiments, R^{b} is methyl. In some embodiments, R^{b} is ethyl. In some embodiments, R^{b} is propyl. In some embodiments, R^{b} is butyl. In some embodiments, R^{b} is pentyl. In some embodiments, R^{b} is hexyl. In some embodiments, R^{b} is isopropyl. In some embodiments, R^{b} is isobutyl. In some embodiments, R^{b} is isopentyl. In some embodiments, R^{b} is isohexyl. In some embodiments, R^{b} is secbutyl. In some embodiments, R^{b} is secpentyl. In some embodiments, R^{b} is sechexyl. In some embodiments, R^{b} is tertbutyl.

In some embodiments, R^{b} is C₃-C₁₀ cycloalkyl.

In some embodiments, R^{b} is C₃-C₁₀ cycloalkyl optionally substituted with —OH or C₁-C₆ haloalkyl.

In some embodiments, R^{b} is C₃-C₁₀ cycloalkyl substituted with —OH or C₁-C₆haloalkyl.

In some embodiments, R^{b} is 3- to 10-membered heterocycloalkyl.

In some embodiments, R^{b} is 3- to 10-membered heterocycloalkyl optionally substituted with —OH or C₁-C₆ haloalkyl.

In some embodiments, R^{b} is 3- to 10-membered heterocycloalkyl substituted with —OH or C₁-C₆ haloalkyl.

In some embodiments, R^{b} is H, methyl, ethyl, cyclobutyl, cyclopropyl,

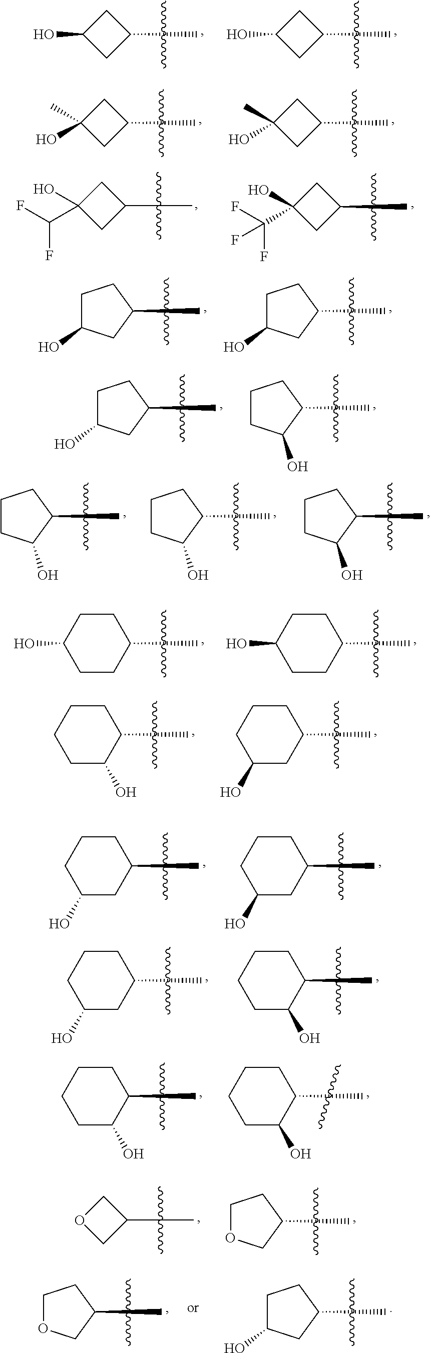

In some embodiments, R^{c} is H, C₁-C₆ alkyl, C₃-C₁₀ cycloalkyl, or 3- to 10-membered heterocycloalkyl.

In some embodiments, R^{c} is H, C₁-C₆ alkyl, C₃-C₁₀ cycloalkyl, or 3- to 10-membered heterocycloalkyl, wherein the C₁-C₆ alkyl, C₃-C₁₀ cycloalkyl, or 3- to 10-membered heterocycloalkyl is optionally substituted with one or more —OH or C₁-C₆ haloalkyl.

In some embodiments, R^{c} is H, C₁-C₆ alkyl, C₃-C₁₀ cycloalkyl, or 3- to 10-membered heterocycloalkyl, wherein the C₁-C₆ alkyl, C₃-C₁₀ cycloalkyl, or 3- to 10-membered heterocycloalkyl is substituted with one or more —OH or C₁-C₆ haloalkyl.

In some embodiments, R^{c} is H.

In some embodiments, R^{c} is C₁-C₆ alkyl.

In some embodiments, R^{c} is C₁-C₆ alkyl optionally substituted with —OH or C₁-C₆ haloalkyl.

In some embodiments, R^{c} is C₁-C₆ alkyl substituted with —OH or C₁-C₆ haloalkyl.

In some embodiments, R^{c} is methyl. In some embodiments, R^{c} is ethyl. In some embodiments, R^{c} is propyl. In some embodiments, R^{c} is butyl. In some embodiments, R^{c} is pentyl. In some embodiments, R^{c} is hexyl. In some embodiments, R^{c} is isopropyl. In some embodiments, R^{c} is isobutyl. In some embodiments, R^{c} is isopentyl. In some embodiments, R^{c} is isohexyl. In some embodiments, R^{c} is secbutyl. In some embodiments, R^{c} is secpentyl. In some embodiments, R^{c} is sechexyl. In some embodiments, R^{c} is tertbutyl.

In some embodiments, R^{c} is C₃-C₁₀ cycloalkyl.

In some embodiments, R^{c} is C₃-C₁₀ cycloalkyl optionally substituted with —OH or C₁-C₆ haloalkyl.

In some embodiments, R^{c} is C₃-C₁₀ cycloalkyl substituted with —OH or C₁-C₆ haloalkyl.

In some embodiments, R^{c} is 3- to 10-membered heterocycloalkyl.

In some embodiments, R^{c} is 3- to 10-membered heterocycloalkyl optionally substituted with —OH or C₁-C₆ haloalkyl.

In some embodiments, R^{c} is 3- to 10-membered heterocycloalkyl substituted with —OH or C₁-C₆ haloalkyl.

In some embodiments, R^{c} is H, methyl, ethyl, cyclobutyl, cyclopropyl,

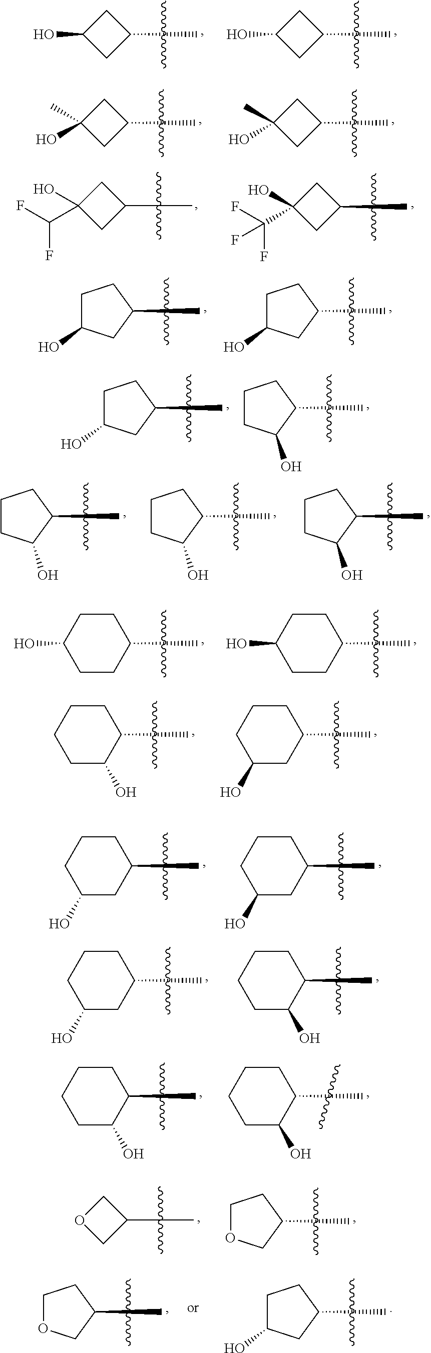

In some embodiments, each R^{a1} independently is oxo.

In some embodiments, each R^{a1} independently is halo.

In some embodiments, each R^{a1} independently is F, Cl, Br, or I. In some embodiments, each R^{a1} independently is F, Cl, or Br. In some embodiments, each R^{a1} independently is F or Cl.

In some embodiments, each R^{a1} independently is F. In some embodiments, each R^{a1} independently is Cl. In some embodiments, each R^{a1} independently is Br. In some embodiments, each R^{a1} independently is I.

In some embodiments, each R^{a1} independently is cyano.

In some embodiments, each R^{a1} independently is —OH.

In some embodiments, each R^{a1} independently is —O(C₃-C₁₀ cycloalkyl).

In some embodiments, each R^{a1} independently is —O(C₃-C₁₀ cycloalkyl), wherein the C₃-C₁₀ cycloalkyl is optionally substituted with one or more R^{a2}.

In some embodiments, each R^{a1} independently is —O(C₃-C₁₀ cycloalkyl), wherein the C₃-C₁₀ cycloalkyl is substituted with one or more R^{a2}.

In some embodiments, each R^{a1} independently is —NH₂.

In some embodiments, each R^{a1} independently is —NH(C₁-C₆ alkyl).

In some embodiments, each R^{a1} independently is —NH(C₁-C₆ alkyl), wherein the C₁-C₆ alkyl is optionally substituted with one or more R^{a2}.

In some embodiments, each R^{a1} independently is —NH(C₁-C₆ alkyl), wherein the C₁-C₆ alkyl is substituted with one or more R^{a2}.

In some embodiments, each R^{a1} independently is —N(C₁-C₆ alkyl)₂.

In some embodiments, each R^{a1} independently is —N(C₁-C₆ alkyl)₂, wherein the C₁-C₆ alkyl is optionally substituted with one or more R^{a2}.

In some embodiments, each R^{a1} independently is —N(C₁-C₆ alkyl)₂, wherein the C₁-C₆ alkyl is substituted with one or more R^{a2}.

In some embodiments, each R^{a1} independently is —C(O)OH.

In some embodiments, each R^{a1} independently is —C(O)O(C₁-C₆ alkyl).

In some embodiments, each R^{a1} independently is —C(O)O(C₁-C₆ alkyl), wherein the C₁-C₆ alkyl is optionally substituted with one or more R^{a2}.

In some embodiments, each R^{a1} independently is —C(O)O(C₁-C₆ alkyl), wherein the C₁-C₆ alkyl is substituted with one or more R^{a2}.

In some embodiments, each R^{a1} independently is C₁-C₆ alkyl.

In some embodiments, each R^{a1} independently is C₁-C₆ alkyl optionally substituted with one or more R^{a2}.

In some embodiments, each R^{a1} independently is C₁-C₆ alkyl substituted with one or more R^{a2}.

In some embodiments, each R^{a1} independently is methyl. In some embodiments, each R^{a1} independently is ethyl. In some embodiments, each R^{a1} independently is propyl. In some embodiments, each R^{a1} independently is butyl. In some embodiments, each R^{a1} independently is pentyl. In some embodiments, each R^{a1} independently is hexyl. In some embodiments, each R^{a1} independently is isopropyl. In some embodiments, each R^{a1} independently is isobutyl. In some embodiments, each R^{a1} independently is isopentyl. In some embodiments, each R^{a1} independently is isohexyl. In some embodiments, each R^{a1} independently is secbutyl. In some embodiments, each R^{a1} independently is R^{a1} is secpentyl. In some embodiments, each R^{a1} independently is sechexyl. In some embodiments, each R^{a1} independently is tertbutyl.

In some embodiments, each R^{a1} independently is C₂-C₆ alkenyl.

In some embodiments, each R^{a1} independently is C₂-C₆ alkenyl optionally substituted with one or more R^{a2}.

In some embodiments, each R^{a1} independently is C₂-C₆ alkenyl substituted with one or more R^{a2}.

In some embodiments, each R^{a1} independently is C₂-C₆ alkynyl.

In some embodiments, each R^{a1} independently is C₂-C₆ alkynyl optionally substituted with one or more R^{a2}.

In some embodiments, each R^{a1} independently is C₂-C₆ alkynyl substituted with one or more R^{a2}.

In some embodiments, each R^{a1} independently is C₁-C₆ haloalkyl.

In some embodiments, each R^{a1} independently is C₁-C₆ haloalkyl optionally substituted with one or more R^{a2}.

In some embodiments, each R^{a1} independently is C₁-C₆ haloalkyl substituted with one or more R^{a2}.

In some embodiments, each R^{a1} independently is halomethyl. In some embodiments, each R^{a1} independently is haloethyl. In some embodiments, each R^{a1} independently is halopropyl. In some embodiments, each R^{a1} independently is halobutyl. In some embodiments, each R^{a1} independently is halopentyl. In some embodiments, each R^{a1} independently is halohexyl.

In some embodiments, each R^{a1} independently is C₁-C₆ alkoxy.

In some embodiments, each R^{a1} independently is C₁-C₆ alkoxy optionally substituted with one or more R^{a2}.

In some embodiments, each R^{a1} independently is C₁-C₆ alkoxy substituted with one or more R^{a2}.

In some embodiments, each R^{a1} independently is C₁-C₆ haloalkoxy.

In some embodiments, each R¹ independently is C₁-C₆ haloalkoxy optionally substituted with one or more R^{a2}.

In some embodiments, each R^{a1} independently is C₁-C₆ haloalkoxy substituted with one or more R^{a2}.

In some embodiments, each R^{a1} independently is —O—(C₃-C₁₀ cycloalkyl).

In some embodiments, each R^{a1} independently is —O—(C₃-C₁₀ cycloalkyl) optionally substituted with one or more R^{a2}.

In some embodiments, each R^{a1} independently is —O—(C₃-C₁₀ cycloalkyl) substituted with one or more R^{a2}.

In some embodiments, each R^{a1} independently is C₃-C₁₀ cycloalkyl.

In some embodiments, each R^{a1} independently is C₃-C₁₀ cycloalkyl optionally substituted with one or more R^{a2}.

In some embodiments, each R^{a1} independently is C₃-C₁₀ cycloalkyl substituted with one or more R^{a2}.

In some embodiments, each R^{a1} independently is C₃-C₇ cycloalkyl.

In some embodiments, each R^{a1} independently is C₃-C₇ cycloalkyl optionally substituted with one or more R^{a2}.

In some embodiments, each R^{a1} independently is C₃-C₇ cycloalkyl substituted with one or more R^{a2}.

In some embodiments, each R^{a1} independently is 3- to 10-membered heterocycloalkyl.

In some embodiments, each R^{a1} independently is 3- to 10-membered heterocycloalkyl optionally substituted with one or more R^{a2}.

In some embodiments, each R^{a1} independently is 3- to 10-membered heterocycloalkyl substituted with one or more R^{a2}.

In some embodiments, each R^{a1} independently is 3- to 7-membered heterocycloalkyl.

In some embodiments, each R^{a1} independently is 3- to 7-membered heterocycloalkyl optionally substituted with one or more R^{a2}.

In some embodiments, each R^{a1} independently is 3- to 7-membered heterocycloalkyl substituted with one or more R^{a2}.

In some embodiments, each R^{a1} independently is C₆-C₁₀ aryl.

In some embodiments, each R^{a1} independently is C₆-C₁₀ aryl optionally substituted with one or more R^{a2}.

In some embodiments, each R^{a1} independently is C₆-C₁₀ aryl substituted with one or more R^{a2}.

In some embodiments, each R^{a1} independently is C₆ aryl.

In some embodiments, each R^{a1} independently is C₆ aryl optionally substituted with one or more R^{a2}.

In some embodiments, each R^{a1} independently is C₆ aryl substituted with one or more R^{a2}.

In some embodiments, each R^{a1} independently is 5- to 10-membered heteroaryl.

In some embodiments, each R^{a1} independently is 5- to 10-membered heteroaryl optionally substituted with one or more R^{a2}.

In some embodiments, each R^{a1} independently is 5- to 10-membered heteroaryl substituted with one or more R^{a2}.

In some embodiments, each R^{a1} independently is 5- or 6-membered heteroaryl.

In some embodiments, each R^{a1} independently is 5- or 6-membered heteroaryl optionally substituted with one or more R^{a2}.

In some embodiments, each R^{a1} independently is 5- or 6-membered heteroaryl substituted with one or more R^{a2}.

In some embodiments, each R^{a1} independently is oxo, —OH, —CH₂OH, —CHF₂, —CF₃, —F, —Cl, —CN, —OCH₃, —OCHF₂, —OCF₃, —NHCH₃, —N(CH₃)₂, —CH₃, —CH₂CF₃, —CH₂CN,

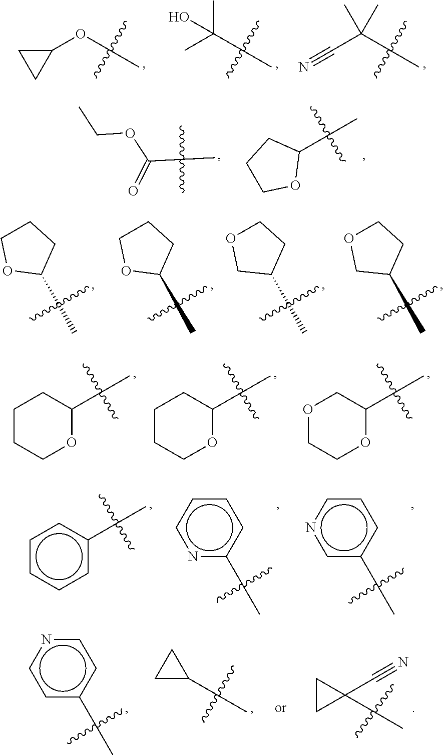

In some embodiments, each R^{a}2 independently is cyano or —OH.

In some embodiments, each R^{a}2 independently is oxo.

In some embodiments, each R^{a}2 independently is halo.

In some embodiments, each R^{a2} independently is F, Cl, Br, or I. In some embodiments, each R^{a2} independently is F, Cl, or Br. In some embodiments, each R² independently is F or Cl.

In some embodiments, each R^{a2} independently is F. In some embodiments, each R^{a2}independently is Cl. In some embodiments, each R^{a2} independently is Br. In some embodiments, each R^{a}2 independently is I.

In some embodiments, each R^{a}2 independently is cyano.

In some embodiments, each R^{a}2 independently is —OH.

In some embodiments, each R^{a}2 independently is —NH₂.

In some embodiments, R³ is C₆-C₁₀ aryl.

In some embodiments, R³ is C₆-C₁₀ aryl optionally substituted with one or more R^{3a}

In some embodiments, R³ is C₆-C₁₀ aryl substituted with one or more R^{3a}

In some embodiments, R³ is C₆ aryl.

In some embodiments, R³ is C₆ aryl optionally substituted with one or more R^{3a}

In some embodiments, R³ is C₆ aryl substituted with one or more R^{3a}

In some embodiments, R³ is 6- to 10-membered heteroaryl.

In some embodiments, R³ is 6- to 10-membered heteroaryl optionally substituted with one or more R^{3a}

In some embodiments, R³ is 6- to 10-membered heteroaryl substituted with one or more R^{3a}

In some embodiments, R³ is 6-membered heteroaryl.

In some embodiments, R³ is 6-membered heteroaryl optionally substituted with one or more R^{3a}

In some embodiments, R³ is 6-membered heteroaryl substituted with one or more R^{3a}

In some embodiments, R³ is:

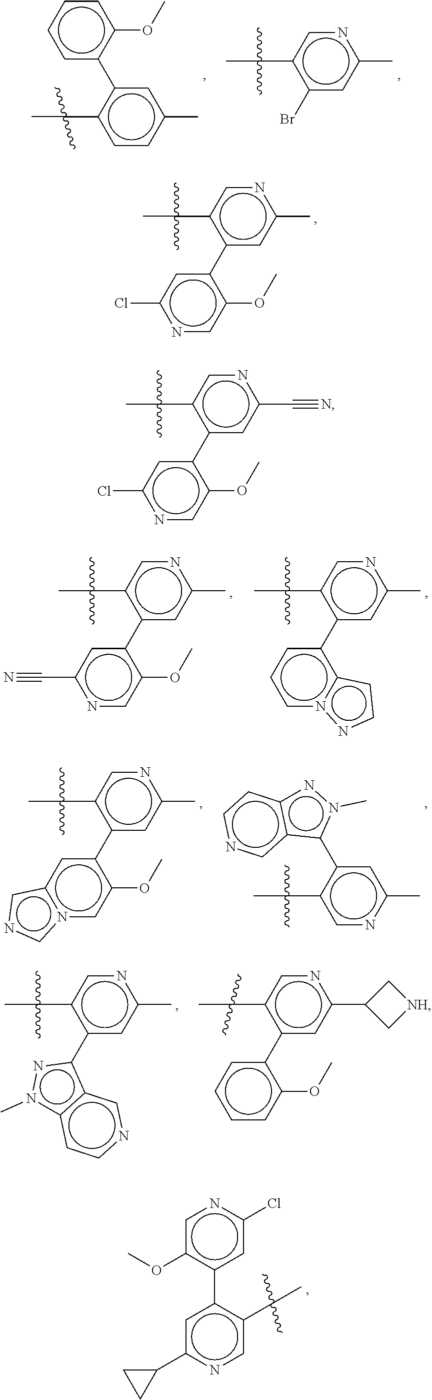

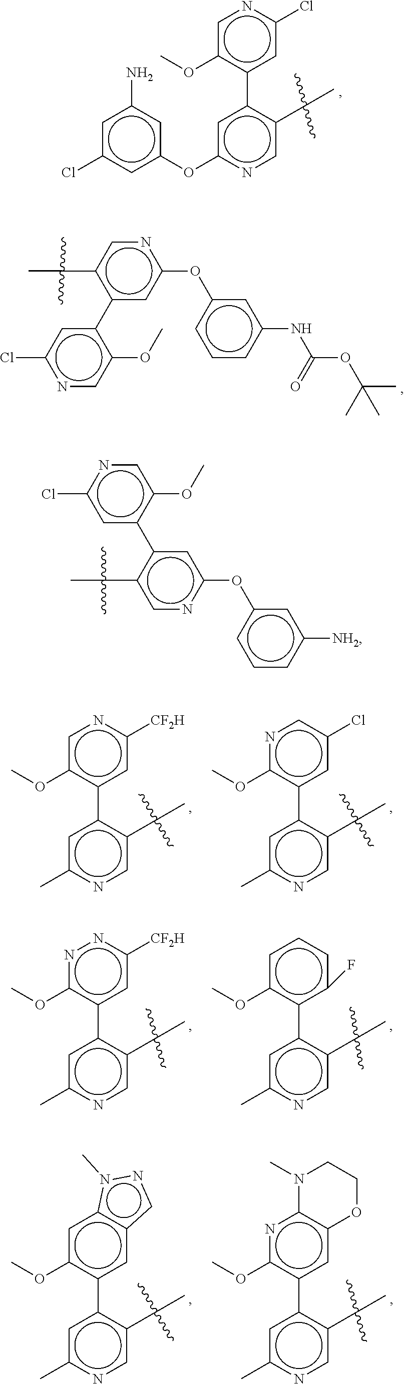

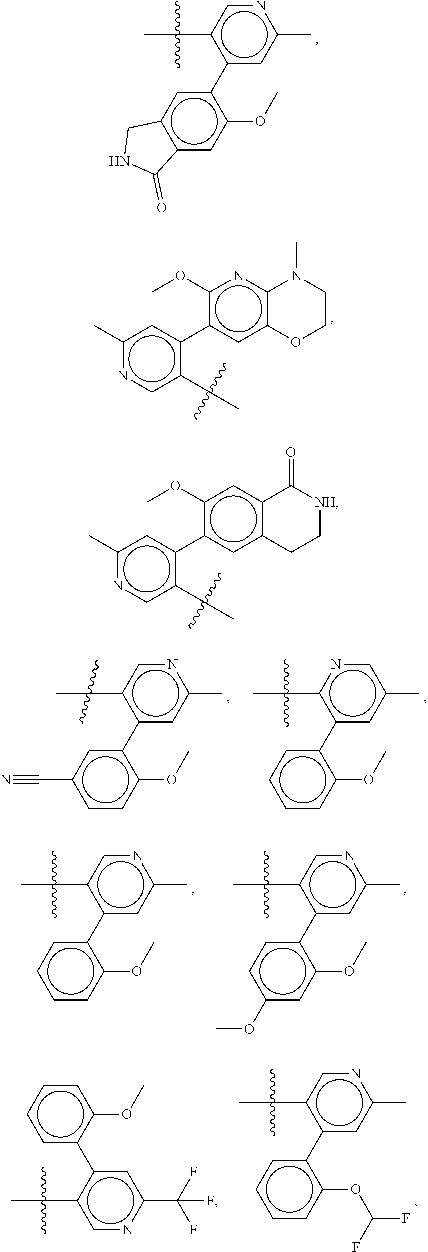

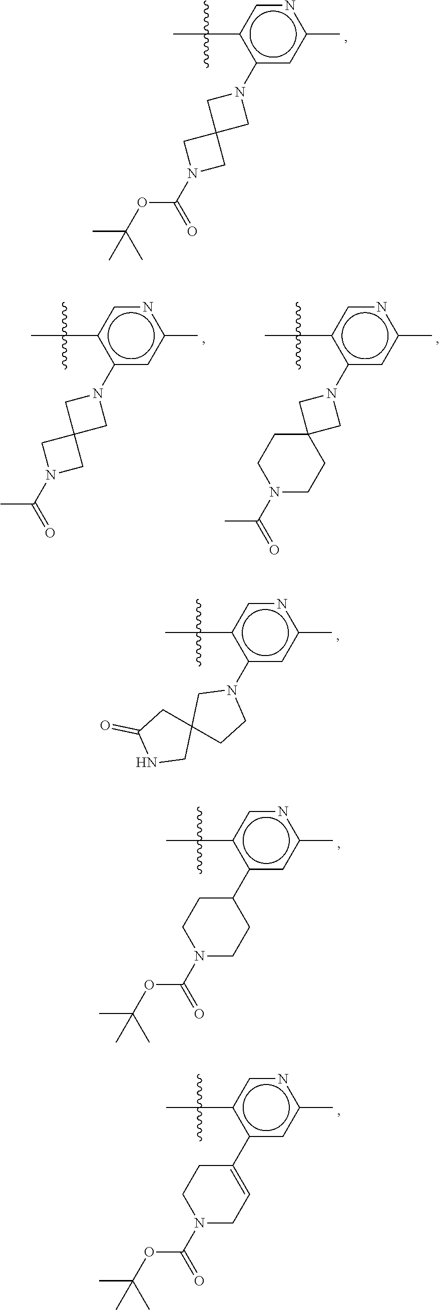

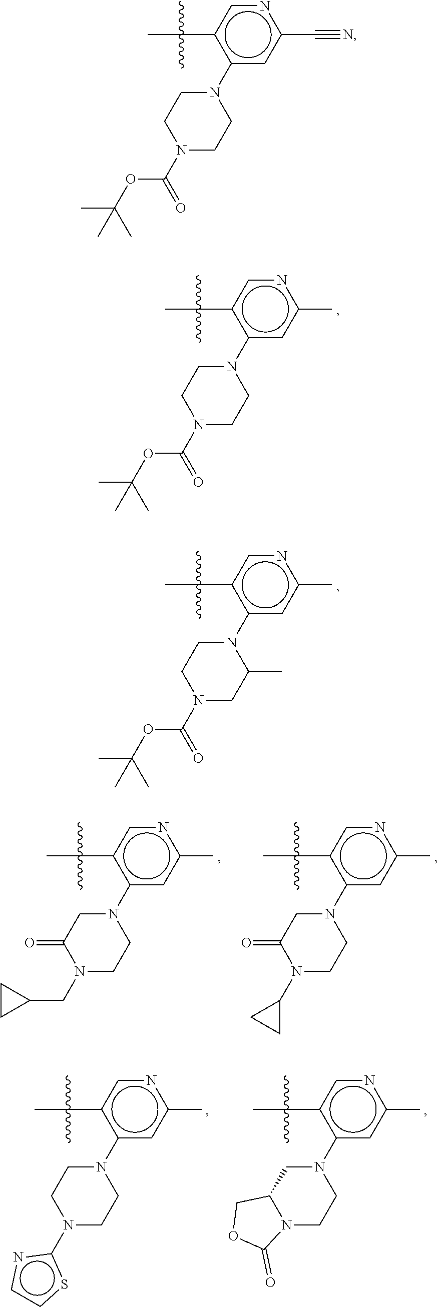

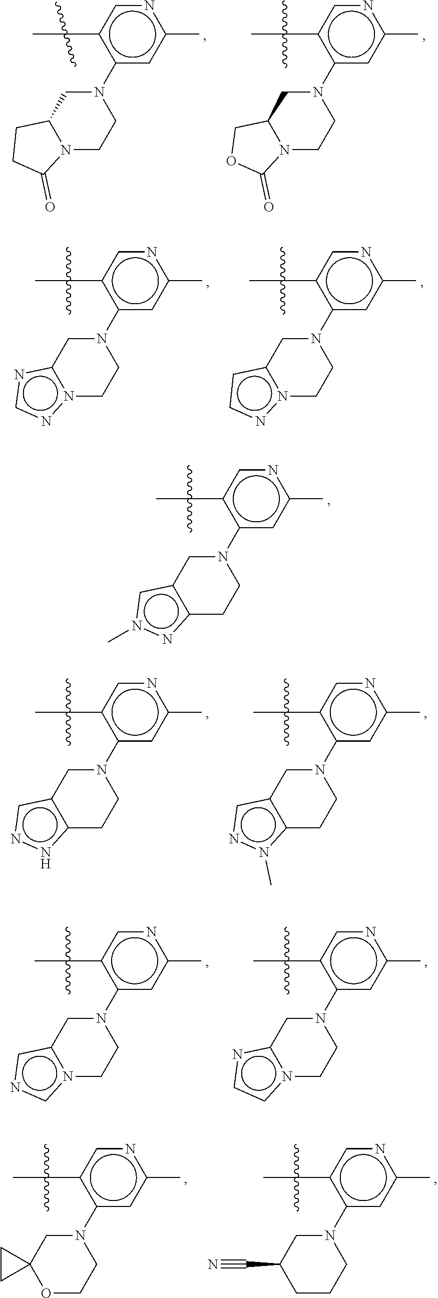

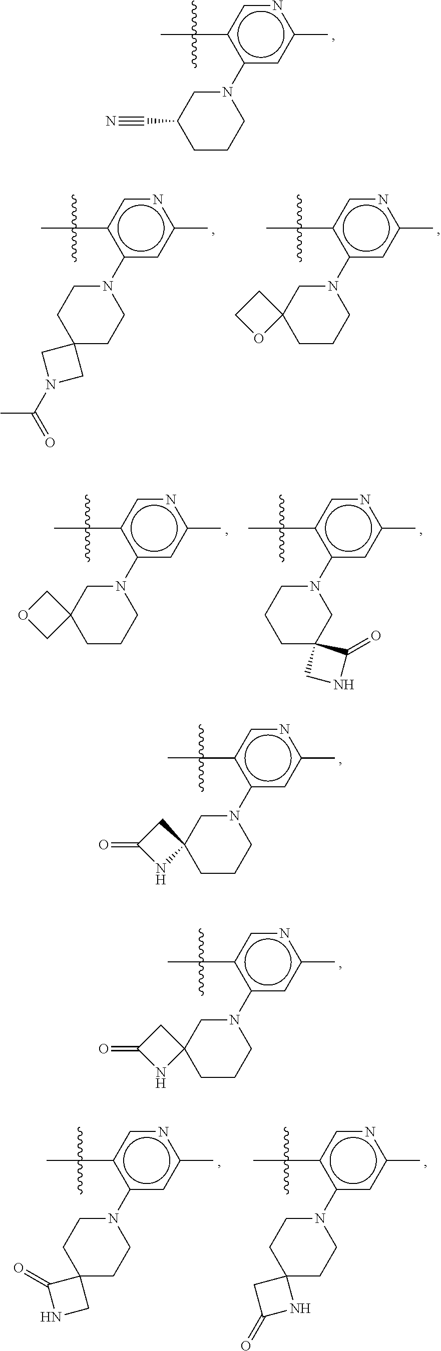

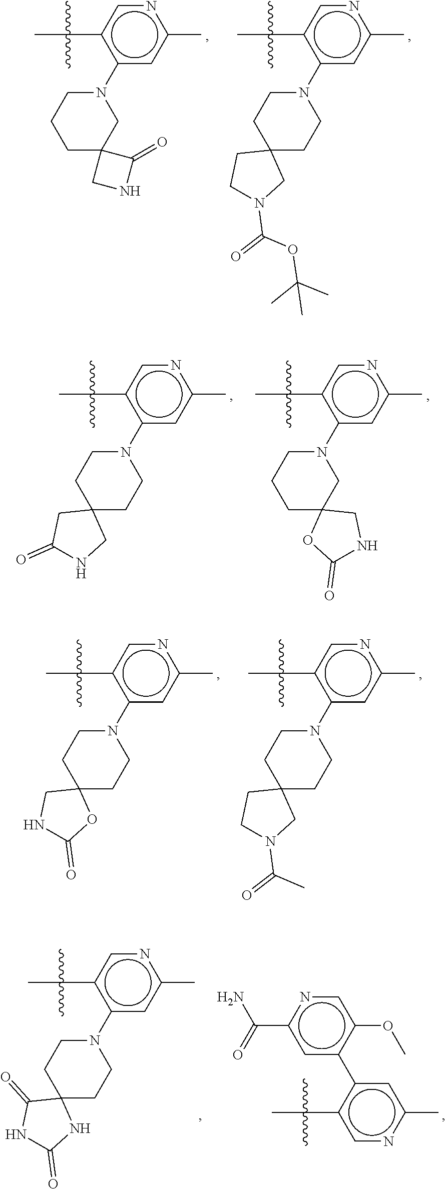

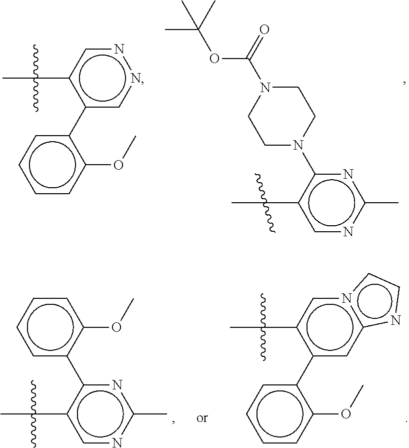

In some embodiments, each R^{3a} independently is halo.

In some embodiments, each R^{3a} independently is cyano, C₁-C₆ alkyl, C₁-C₆ haloalkyl, 3- to 10-membered heterocycloalkyl, C₆-C₁₀ aryl, or 5- to 10-membered heteroaryl.

In some embodiments, each R^{3a} independently is cyano, C₁-C₆ alkyl, C₁-C₆ haloalkyl, 3-to 10-membered heterocycloalkyl, C₆-C₁₀ aryl, or 5- to 10-membered heteroaryl, wherein the C₁-C₆ alkyl, C₁-C₆ haloalkyl, C₃-C₁₀ cycloalkyl, 3- to 10-membered heterocycloalkyl, C₆-C₁₀ aryl, or 5- to 10-membered heteroaryl is optionally substituted with one or more R^{3a1}.

In some embodiments, each R^{3a} independently is cyano, C₁-C₆ alkyl, C₁-C₆ haloalkyl, 3-to 10-membered heterocycloalkyl, C₆-C₁₀ aryl, or 5- to 10-membered heteroaryl, wherein the C₁-C₆ alkyl, C₁-C₆ haloalkyl, C₃-C₁₀ cycloalkyl, 3- to 10-membered heterocycloalkyl, C₆-C₁₀ aryl, or 5- to 10-membered heteroaryl is substituted with one or more R

In some embodiments, each R^{3a} independently is F, Cl, Br, or I. In some embodiments, each R^{3a} independently is F, Cl, or Br. In some embodiments, each R^{3a} independently is F or Cl.

In some embodiments, each R^{3a} independently is F. In some embodiments, each R^{3a} independently is Cl. In some embodiments, each R^{3a} independently is Br. In some embodiments, each R^{3a} independently is I.

In some embodiments, each R^{3a} independently is cyano.

In some embodiments, each R^{3a} independently is —OH.

In some embodiments, each R^{3a} independently is —NH₂.

In some embodiments, each R^{3a} independently is C₁-C₆ alkyl.

In some embodiments, each R^{3a} independently is C₁-C₆ alkyl optionally substituted with one or more R^{3a1}.

In some embodiments, each R^{3a} independently is C₁-C₆ alkyl substituted with one or more R^{3a1}.

In some embodiments, each R^{3a} independently is methyl. In some embodiments, each R^{3a} independently is ethyl. In some embodiments, each R^{3a} independently is propyl. In some embodiments, each R^{3a} independently is butyl. In some embodiments, each R^{3a} independently is pentyl. In some embodiments, each R^{3a} independently is hexyl. In some embodiments, each R^{3a} independently is isopropyl. In some embodiments, each R^{3a} independently is isobutyl. In some embodiments, each R^{3a} independently is isopentyl. In some embodiments, each R^{3a} independently is isohexyl. In some embodiments, each R^{3a} independently is secbutyl. In some embodiments, each R^{3a} independently is secpentyl. In some embodiments, each R^{3a} independently is sechexyl. In some embodiments, each R^{3a} independently is tertbutyl.

In some embodiments, each R^{3a} independently is C₂-C₆ alkenyl.

In some embodiments, each R^{3a} independently is C₂-C₆ alkenyl optionally substituted with one or more R^{3a1}.

In some embodiments, each R^{3a} independently is C₂-C₆ alkynyl.

In some embodiments, each R^{3a} independently is C₂-C₆ alkynyl optionally substituted with one or more R^{3a1}.

In some embodiments, each R^{3a} independently is C₁-C₆ haloalkyl.

In some embodiments, each R^{3a} independently is C₁-C₆ haloalkyl optionally substituted with one or more R^{3a1}.

In some embodiments, each R^{3a} independently is C₁-C₆ haloalkyl substituted with one or more R^{3a1}.

In some embodiments, each R^{3a} independently is halomethyl. In some embodiments, each R^{3a} independently is haloethyl. In some embodiments, each R^{3a} independently is halopropyl. In some embodiments, each R^{3a} independently is halobutyl. In some embodiments, each R^{3a} independently is halopentyl. In some embodiments, each R^{3a} independently is halohexyl.

In some embodiments, each R^{3a} independently is C₃-C₁₀ cycloalkyl.

In some embodiments, each R^{3a} independently is C₃-C₁₀ cycloalkyl optionally substituted with one or more R^{3a1}.

In some embodiments, each R^{3a} independently is C₃-C₁₀ cycloalkyl substituted with one or more R^{3a1}.

In some embodiments, each R^{3a} independently is C₃-C₇ cycloalkyl.

In some embodiments, each R^{3a} independently is C₃-C₇ cycloalkyl optionally substituted with one or more R^{3a1}.

In some embodiments, each R^{3a} independently is C₃-C₇ cycloalkyl substituted with one or more R^{3a1}.

In some embodiments, each R^{3a} independently is 3- to 10-membered heterocycloalkyl.

In some embodiments, each R^{3a} independently is 3- to 10-membered heterocycloalkyl optionally substituted with one or more R^{3a1}.

In some embodiments, each R^{3a} independently is 3- to 10-membered heterocycloalkyl substituted with one or more R^{3a1}.

In some embodiments, each R^{3a} independently is 3- to 7-membered heterocycloalkyl.

In some embodiments, each R^{3a} independently is 3- to 7-membered heterocycloalkyl optionally substituted with one or more R^{3a1}.

In some embodiments, each R^{3a} independently is 3- to 7-membered heterocycloalkyl substituted with one or more R^{3a1}.

In some embodiments, each R^{3a} independently is —O(C₆-C₁₀ aryl).

In some embodiments, each R^{3a} independently is —O(C₆-C₁₀ aryl) optionally substituted with one or more R^{3a1}.

In some embodiments, each R^{3a} independently is —O(C₆-C₁₀ aryl) substituted with one or more R^{3a1}.

In some embodiments, each R^{3a} independently is C₆-C₁₀ aryl.

In some embodiments, each R^{3a} independently is C₆-C₁₀ aryl optionally substituted with one or more R^{3a1}.

In some embodiments, each R^{3a} independently is C₆-C₁₀ aryl substituted with one or more R^{3a1}.

In some embodiments, each R^{3a} independently is C₆ aryl.

In some embodiments, each R^{3a} independently is C₆ aryl optionally substituted with one or more R^{3a1}.

In some embodiments, each R^{3a} independently is C₆ aryl substituted with one or more R^{3a1}.

In some embodiments, each R^{3a} independently is 5- to 10-membered heteroaryl.

In some embodiments, each R^{3a} independently is 5- to 10-membered heteroaryl optionally substituted with one or more R^{3a1}.

In some embodiments, each R^{3a} independently is 5- to 10-membered heteroaryl substituted with one or more R^{3a1}.

In some embodiments, each R^{3a} independently is 5- or 6-membered heteroaryl.

In some embodiments, each R^{3a} independently is 5- or 6-membered heteroaryl optionally substituted with one or more R^{3a1}.

In some embodiments, each R^{3a} independently is 5- or 6-membered heteroaryl substituted with one or more R^{3a1}.

In some embodiments, each R^{3a} independently is —Br, —CH₃, —CF₃, —CN,

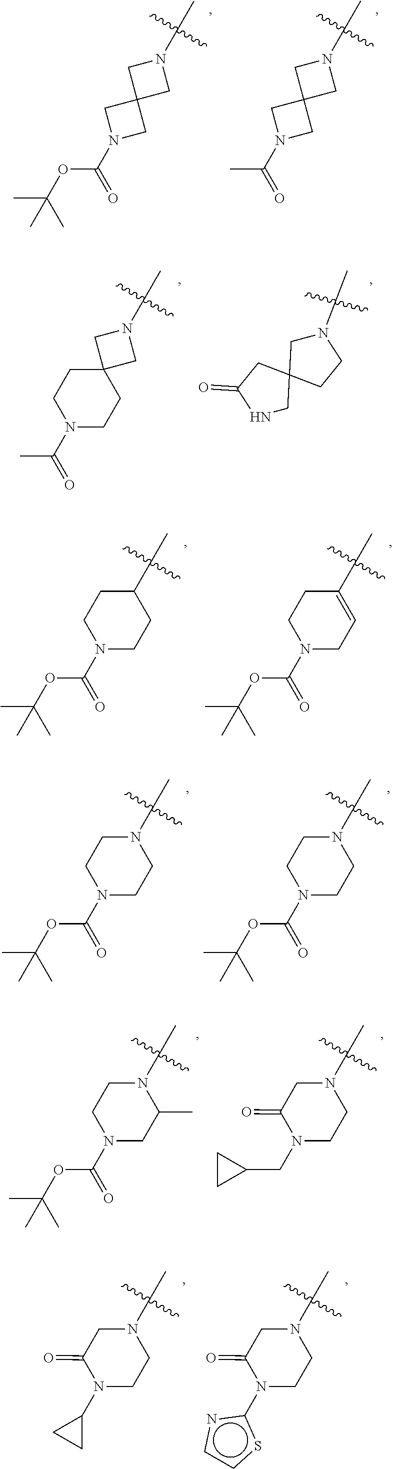

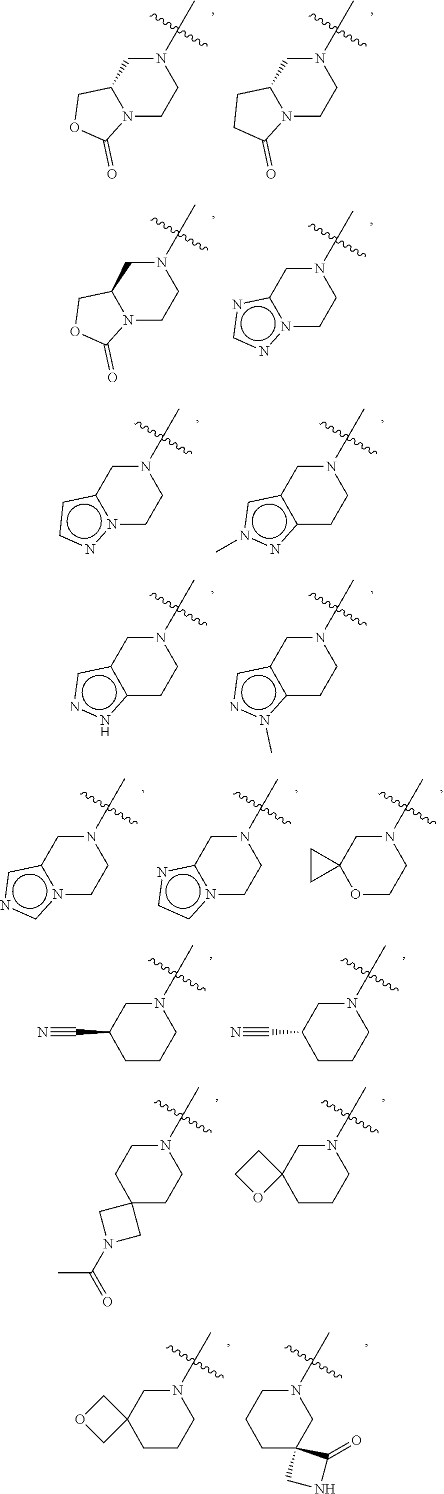

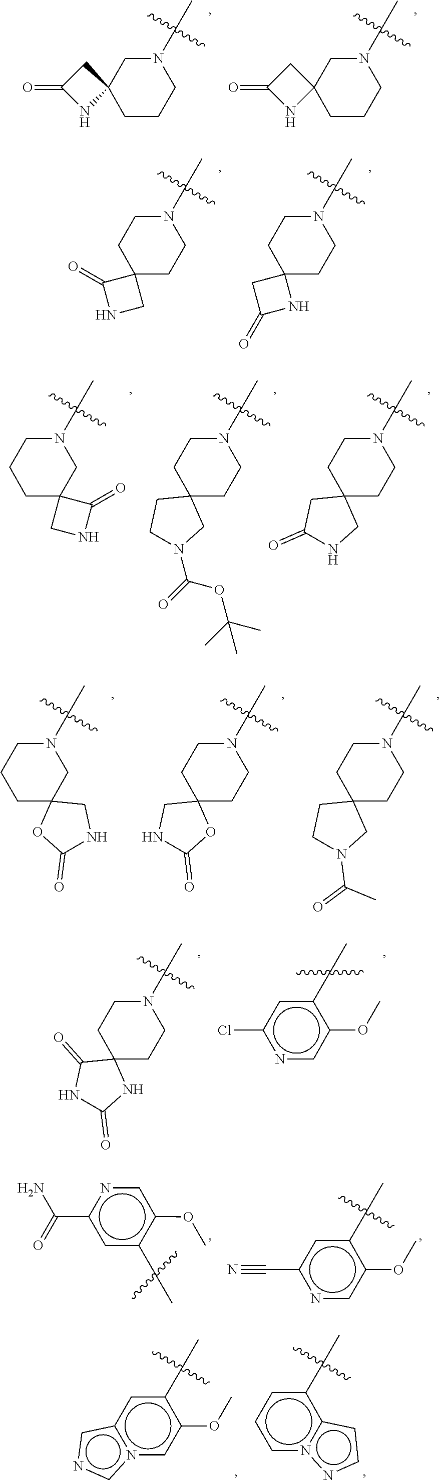

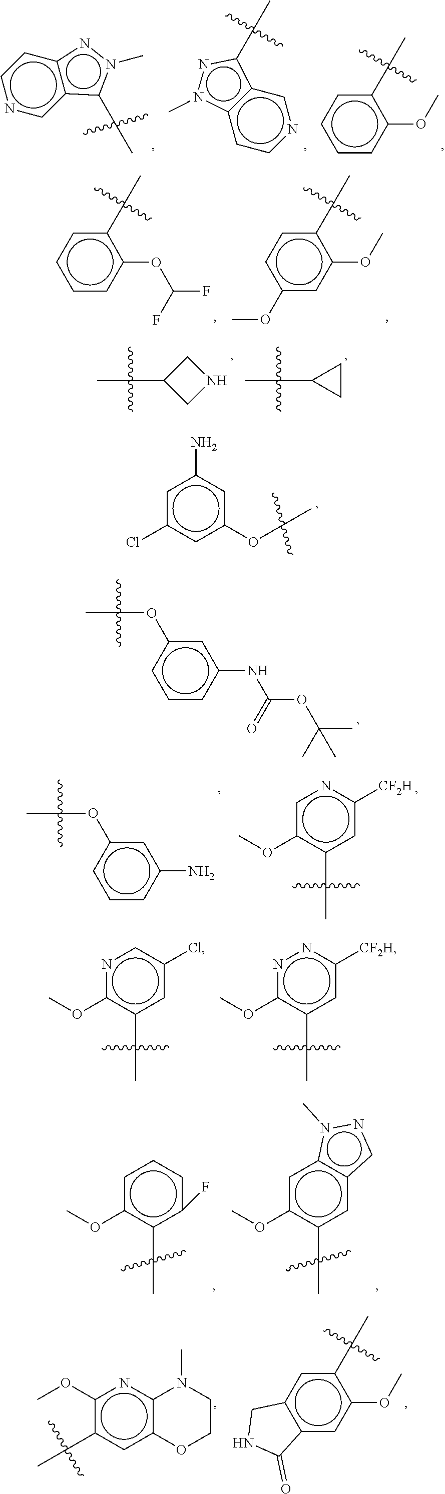

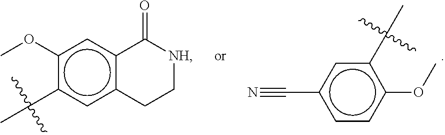

In some embodiments, each R^{3a1} independently is oxo, halo, cyano, —NH₂, —NH—C(O)O(C₁-C₆ alkyl), —C(O)(C₁-C₆ alkyl), —C(O)NH₂, —C(O)(O—(C₁-C₆ alkyl)), C₁-C₆ alkyl optionally substituted with one or more C₃-C₁₀ cycloalkyl, C₁-C₆ alkoxy, —O(C₁-C₆ haloalkyl), C₃-C₁₀ cycloalkyl, or 5- to 10-membered heteroaryl.

In some embodiments, each R^{3a1} independently is oxo, halo, cyano, —C(O)(C₁-C₆ alkyl), —C(O)NH₂, —C(O)(O—(C₁-C₆ alkyl)), C₁-C₆ alkyl optionally substituted with one or more C₃-C₁₀ cycloalkyl, C₁-C₆ alkoxy, —O(C₁-C₆ haloalkyl), C₃-C₁₀ cycloalkyl, or 5- to 10-membered heteroaryl.

In some embodiments, each R^{3a1} independently is oxo, halo, cyano, —C(O)(C₁-C₆ alkyl), —C(O)NH₂, —C(O)(O—(C₁-C₆ alkyl)), C₁-C₆ alkyl substituted with one or more C₃-C₁₀ cycloalkyl, C₁-C₆ alkoxy, —O(C₁-C₆ haloalkyl), C₃-C₁₀ cycloalkyl, or 5- to 10-membered heteroaryl.

In some embodiments, each R^{3a1} independently is oxo.

In some embodiments, each R^{3a1} independently is halo.

In some embodiments, each R^{3a1} independently is F, Cl, Br, or I. In some embodiments, each R^{3a1} independently is F, Cl, or Br. In some embodiments, each R^{3a1} independently is F or Cl.

In some embodiments, each R^{3a1} independently is F. In some embodiments, each R^{3a1} independently is Cl. In some embodiments, each R^{3a1} independently is Br. In some embodiments, each R^{3a1} independently is I.

In some embodiments, each R^{3a1} independently is cyano.

In some embodiments, each R^{3a1} independently is —NH₂.

In some embodiments, each R^{3a1} independently is —NH—C(O)O(C₁-C₆ alkyl).

In some embodiments, each R^{3a1} independently is —C(O)(C₁-C₆ alkyl).

In some embodiments, each R^{3a1} independently is —C(O)(O—(C₁-C₆ alkyl)).

In some embodiments, each R^{3a1} independently —C(O)NH₂.

In some embodiments, each R^{3a1} independently is C₁-C₆ alkyl.

In some embodiments, each R^{3a1} independently is C₁-C₆ alkyl optionally substituted with one or more C₃-C₁₀ cycloalkyl.

In some embodiments, each R^{3a1} independently is C₁-C₆ alkyl substituted with one or more C₃-C₁₀ cycloalkyl.

In some embodiments, each R^{3a1} independently is methyl. In some embodiments, each R^{3a1} independently is ethyl. In some embodiments, each R^{3a1} independently is propyl. In some embodiments, each R^{3a1} independently is butyl. In some embodiments, each R^{3a1} independently is pentyl. In some embodiments, each R^{3a1} independently is hexyl. In some embodiments, each R^{3a1} independently is isopropyl. In some embodiments, each R^{3a1} independently is isobutyl. In some embodiments, each R^{3a1} independently is isopentyl. In some embodiments, each R^{3a1} independently is isohexyl. In some embodiments, each R^{3a1} independently is secbutyl. In some embodiments, each R^{3a1} independently is secpentyl. In some embodiments, each R^{3a1} independently is sechexyl. In some embodiments, each R^{3a1} independently is tertbutyl.

In some embodiments, each R^{3a1} independently is C₂-C₆ alkenyl.

In some embodiments, each R^{3a1} independently is C₂-C₆ alkynyl.

In some embodiments, each R^{3a1} independently is C₁-C₆ haloalkyl.

In some embodiments, each R^{3a1} independently is halomethyl. In some embodiments, each R^{3a1} independently is haloethyl. In some embodiments, each R^{3a1} independently is halopropyl. In some embodiments, each R^{3a1} independently is halobutyl. In some embodiments, each R^{3a1} independently is halopentyl. In some embodiments, each R^{3a1} independently is halohexyl.

In some embodiments, each R^{3a1} independently is C₁-C₆ alkoxy.

In some embodiments, each R^{3a1} independently is —O(C₁-C₆ haloalkyl).

In some embodiments, each R^{3a1} independently is C₃-C₁₀ cycloalkyl.

In some embodiments, each R^{3a1} independently is C₆-C₁₀ aryl.

In some embodiments, each R^{3a1} independently is 5- to 10-membered heteroaryl.

In some embodiments, each R^{3a1} independently is oxo, —Cl, —CH₃, —OCH₃, —CF₂H, —NH₂, —NHC(O)OC(CH₃)₃, —OCHF₂, —C(O)OC(CH₃)₃, —C(O)CH₃, —C(O)NH₂, —CN,

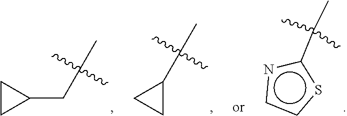

In some embodiments, when R^{3a} is halo and R¹ and R², together with the atoms to which they are attached, form a 6-membered heteroaryl then R^{a} is not —CF₃.

In some embodiments, when R^{3a} is halo and R¹ and R², together with the atoms to which they are attached, form a 6-membered heteroaryl then R^{a} is halo, cyano, —OH, —NH₂, —C(O)N(R^{b})(R^{c}), C₁-C₆ alkyl, C₂-C₆ alkenyl, C₂-C₆ alkynyl, C₃-C₁₀ cycloalkyl, C₆-C₁₀ aryl, 5-to 10-membered heteroaryl, or 3- to 10-membered heterocycloalkyl.

In some embodiments, X¹ is CH or N; and R¹ and R², together with the atoms to which they are attached, form a pyridine optionally substituted with one or more R^{a}.

In some embodiments, X² is N or O; and R¹ and R², together with the atoms to which they are attached, form a pyridine optionally substituted with one or more R^{a}

In some embodiments, when R¹ and R², together with the atoms to which they are attached, form a pyridine optionally substituted with one or more R^{a}, then X¹ is not S.

In some embodiments, when R¹ and R², together with the atoms to which they are attached, form a pyridine optionally substituted with one or more R^{a}, then X² is not S.

In some embodiments, when R¹ and R², together with the atoms to which they are attached, form a pyridine optionally substituted with one or more R^{a}, then

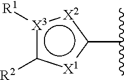

is not

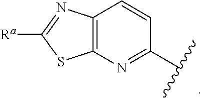

In some embodiments, when R^{a} is

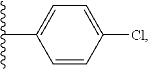 [SMILES: *c1ccc(Cl)cc1]

then R³ is not

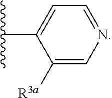

In some embodiments, when R^{a} is

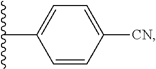 [SMILES: *c1ccc(C#N)cc1]

then R³ is not

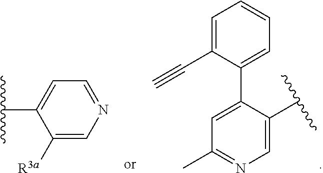

In some embodiments, when R¹ and R², together with the atoms to which they are attached, form a pyrazine substituted with one or more R^{a}, and R^{a} is phenyl substituted with one or more R^{a1}, then R^{a1} is not —Cl, —F, —CN, —O(CH₃), —O(CF₃), —O(CHF₂), —CH₃, or cyclopropyl.

In some embodiments, when R¹ and R², together with the atoms to which they are attached, form a pyrazine substituted with one or more R^{a}, and R^{a} is pyridine substituted with one or more R^{a1}, then R^{a1} is not —Cl, —CN, —CF₃, —O(CH₃), —O(CHF₂), cyclopropyl, —C(CH₃)₂(CN), or —C(CH₃)₂(OH).

In some embodiments, when R¹ and R², together with the atoms to which they are attached, form a pyrazine substituted with one or more R^{a}, then R^{a} is not

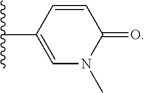 [SMILES: *c1ccc(=O)n(C)c1]

In some embodiments, the compound of Formula (I) is not 5′-methoxy-2′,6-dimethyl-N-(6-(tetrahydrofuran-3-yl)thiazolo[4,5-b]pyrazin-2-yl)-[4,4′-bipyridine]-3-carboxamide.

In some embodiments, the compound is of Formula (I), wherein:
X¹ is CH or N;
X² is N, S, or O;
X³ is C or N;
R¹ and R², together with the atoms to which they are attached, form a C₆-C₁₀ aryl or 5- to 10-membered heteroaryl, wherein the C₆-C₁₀ aryl or 5- to 10-membered heteroaryl is optionally substituted with one or more R^{a};
each R^{a} independently is oxo, halo, cyano, —OH, —NH₂, —C(O)N(R^{b})(R^{c}), C₁-C₆ alkyl, C₂-C₆ alkenyl, C₂-C₆ alkynyl, C₁-C₆ haloalkyl, C₃-C₁₀ cycloalkyl, C₆-C₁₀ aryl, 5- to 10-membered heteroaryl, or 3- to 10-membered heterocycloalkyl, wherein the C₁-C₆ alkyl, C₂-C₆ alkenyl, C₂-C₆ alkynyl, C₁-C₆ haloalkyl, C₃-C₁₀ cycloalkyl, C₆-C₁₀ aryl, 5- to 10-membered heteroaryl, or 3- to 10-membered heterocycloalkyl is optionally substituted with one or more R^{a}; R^{b} and R^{c}, together with the atoms to which they are attached, form a 3- to 10-membered heterocycloalkyl;
each R^{a1} independently is oxo, halo, cyano, —OH, —O(C₃-C₁₀ cycloalkyl), —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl is optionally substituted with one or more R^{a2};
each R^{a2} independently is oxo, halo, cyano, —OH, or —NH₂;
R³ is C₆-C₁₀ aryl or 6- to 10-membered heteroaryl, wherein the C₆-C₁₀ aryl or 6- to 10-membered heteroaryl is substituted with one or more R^{3a};
each R^{3a} independently is halo, cyano, —OH, —NH₂, C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, C₆-C₁₀ aryl, or 5- to 10-membered heteroaryl is optionally substituted with one or more R^{3a1}; and
each R^{3a1} independently is oxo, halo, cyano, —C(O)(C₁-C₆ alkyl), —C(O)(O—(C₁-C₆ alkyl)), C₁-C₆ alkyl optionally substituted with one or more C₃-C₁₀ cycloalkyl, C₂-C₆ alkenyl, C₂-C₆ alkynyl, C₁-C₆ haloalkyl, C₁-C₆ alkoxy, —O(C₁-C₆ haloalkyl), C₃-C₁₀ cycloalkyl, C₆-C₁₀ aryl, or 5- to 10-membered heteroaryl;
wherein, when R^{3a} is halo and R¹ and R², together with the atoms to which they are attached, form a 6-membered heteroaryl then R^{a} is not —CF₃.

In some embodiments, the compound is of Formula (I), wherein:
each R^{a} independently is oxo, halo, —C(O)N(R^{b})(R^{c}), —N(R^{b})C(O)(R^{c}), C₁-C₆ alkyl, C₁-C₆ haloalkyl, C₃-C₁₀ cycloalkyl, C₆-C₁₀ aryl, 5- to 10-membered heteroaryl, or 3- to 10-membered heterocycloalkyl, wherein the C₁-C₆ alkyl, C₁-C₆ haloalkyl, C₃-C₁₀ cycloalkyl, C₆-C₁₀ aryl, 5- to 10-membered heteroaryl, or 3- to 10-membered heterocycloalkyl is optionally substituted with one or more R^{a1};
each R^{a1} independently is oxo, halo, cyano, —OH, —O(C₃-C₁₀ cycloalkyl), —NH(C₁-C₆ alkyl), —N(C₁-C₆ alkyl)₂, —C(O)O(C₁-C₆ alkyl), C₁-C₆ alkyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl, wherein the C₁-C₆ alkyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl is optionally substituted with one or more R^{a2};
each R^{a2} independently is cyano or —OH;
each R^{3a} independently is cyano, C₁-C₆ alkyl, C₁-C₆ haloalkyl, 3- to 10-membered heterocycloalkyl, C₆-C₁₀ aryl, or 5- to 10-membered heteroaryl, wherein the C₁-C₆ alkyl, C₁-C₆ haloalkyl, C₃-C₁₀ cycloalkyl, 3- to 10-membered heterocycloalkyl, C₆-C₁₀ aryl, or 5- to 10-membered heteroaryl is optionally substituted with one or more R^{3a1}; and
each R^{3a1} independently is oxo, halo, cyano, —C(O)(C₁-C₆ alkyl), —C(O)NH₂, —C(O)(0-(C₁-C₆ alkyl)), C₁-C₆ alkyl optionally substituted with one or more C₃-C₁₀ cycloalkyl, C₁-C₆ alkoxy, —O(C₁-C₆ haloalkyl), C₃-C₁₀ cycloalkyl, or 5- to 10-membered heteroaryl.

In some embodiments, the compound is of Formula (I-A), (I-B), or (I-C):

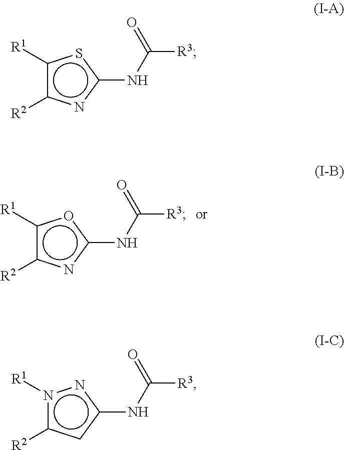

or a pharmaceutically acceptable salt, solvate, clathrate, hydrate, stereoisomer, or tautomer thereof.

In some embodiments, the compound is of Formula (I-D), (I-E), (I-F), (I-G), (I-H), (I-I), (I-J), (I-K), or (I-L):

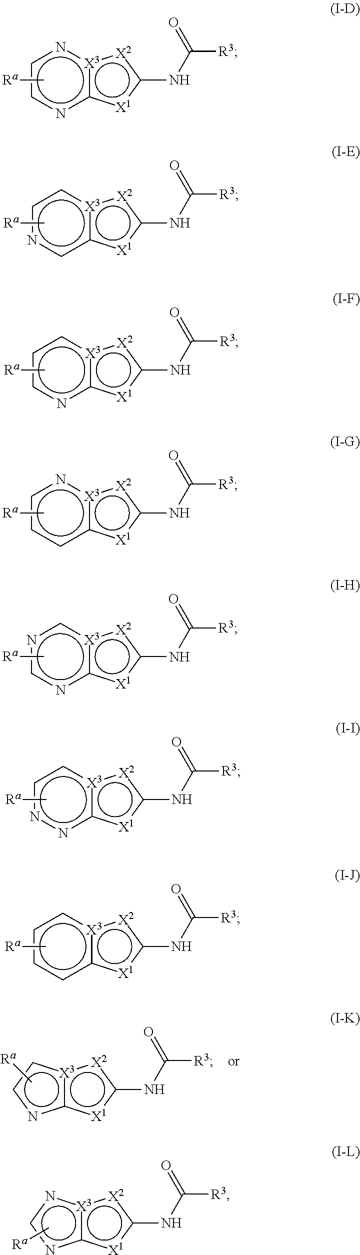

or a pharmaceutically acceptable salt, solvate, clathrate, hydrate, stereoisomer, or tautomer thereof.

In some embodiments of the compound of Formula (I-F), X¹ is not S.

In some embodiments, the compound is of Formula (I-F′):

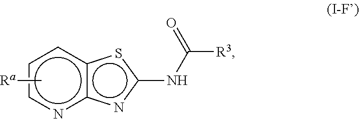

or a pharmaceutically acceptable salt, solvate, clathrate, hydrate, stereoisomer, or tautomer thereof.

In some embodiments, the compound is of Formula (I-M), (I-N), (I-O), (I-P), (I-Q), (I-R), (I-S) (I-T) (I-U) or (I-V):

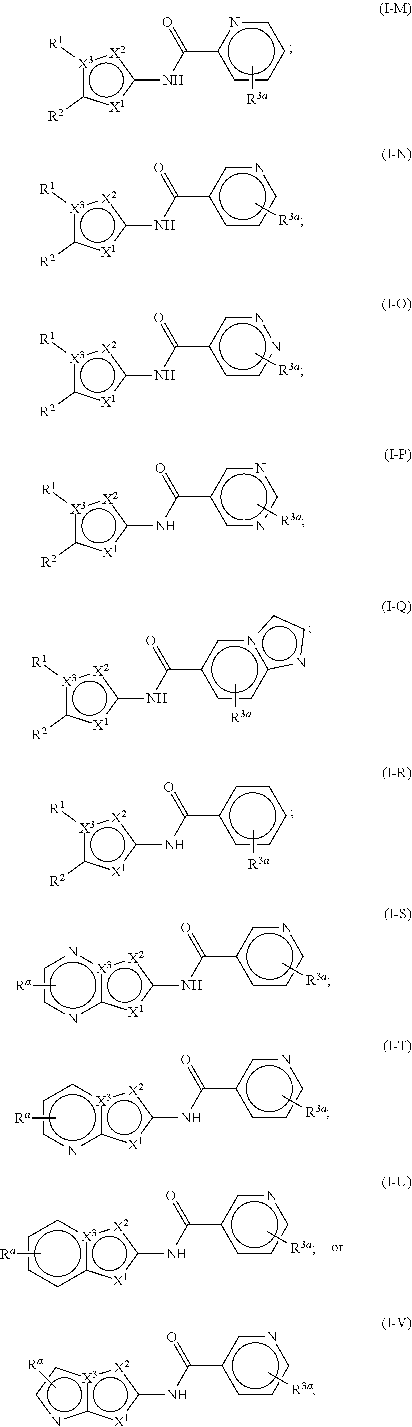

or a pharmaceutically acceptable salt, solvate, enantiomer, diastereomer, racemate or mixture thereof.

In some embodiments of the compound of Formula (I-T), X¹ is not S.

In some embodiments, the compound is of Formula (I-T′)

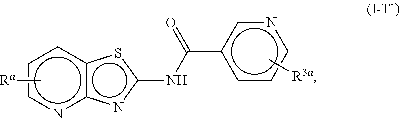

or a pharmaceutically acceptable salt, solvate, enantiomer, diastereomer, racemate or mixture thereof.

In some embodiments, the compound is of Formula (I-W):

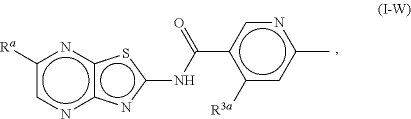

or a pharmaceutically acceptable salt, solvate, enantiomer, diastereomer, racemate or mixture thereof.

In some embodiments, the compound is a compound described in Table 1, or a prodrug or pharmaceutically acceptable salt thereof.

In some embodiments, the compound is a compound described in Table 1, or a pharmaceutically acceptable salt thereof.

In some embodiments, the compound is a prodrug of a compound described in Table 1, or a pharmaceutically acceptable salt thereof.

In some embodiments, the compound is a compound described in Table 1.

In some embodiments, the compound is a compound described in Table 2, or a prodrug or pharmaceutically acceptable salt thereof.

In some embodiments, the compound is a compound described in Table 2, or a pharmaceutically acceptable salt thereof.

In some embodiments, the compound is a prodrug of a compound described in Table 2, or a pharmaceutically acceptable salt thereof.

In some embodiments, the compound is a compound described in Table 2.

In some embodiments, the compound is a compound described in Table 3, or a prodrug or pharmaceutically acceptable salt thereof.

In some embodiments, the compound is a compound described in Table 3, or a pharmaceutically acceptable salt thereof.

In some embodiments, the compound is a prodrug of a compound described in Table 3, or a pharmaceutically acceptable salt thereof.

In some embodiments, the compound is a compound described in Table 3.

In some embodiments, the compound is a compound described in Table 4, or a prodrug or pharmaceutically acceptable salt thereof.

In some embodiments, the compound is a compound described in Table 4, or a pharmaceutically acceptable salt thereof.

In some embodiments, the compound is a prodrug of a compound described in Table 4, or a pharmaceutically acceptable salt thereof.

In some embodiments, the compound is a compound described in Table 4.

Compounds in Tables 1-4 are each assigned a compound number, and any alternative compound (numbers are indicated in parentheses. For compounds with two compound numbers, both compound numbers may be used interchangeably to refer to the same compound.

TABLE 1

| Compound No. | Name | Structure |
|---|---|---|
| 1 | 2′-chloro-N-(6-(4-hydroxypiperidin-1- yl)thiazolo[4,5-b]pyrazin-2-yl)-5′- methoxy-6-methyl-[4,4′-bipyridine]-3- carboxamide | 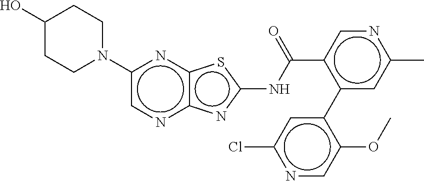 |
| 2 | 2′-chloro-N-(6- (cyclobutanecarboxamido)thiazolo[4,5- b]pyridin-2-yl)-5′-methoxy-6-methyl- [4,4′-bipyridine]-3-carboxamide | 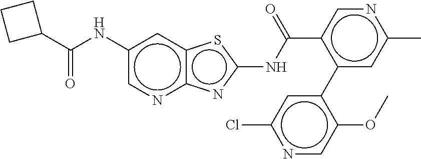 |
| 3 | 2′-chloro-5′-methoxy-6-methyl-N-(6- (pyrrolidine-1-carbonyl)thiazolo[4,5- b]pyrazin-2-yl)-[4,4′-bipyridine]-3- carboxamide | 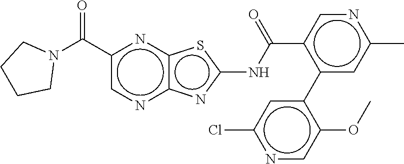 |
| 4 | (Racemic)-2′-chloro-N-{6-[3- (hydroxymethyl)cyclopentyl]- [1,3]thiazolo[4,5-b]pyrazin-2-yl}-5′- methoxy-6-methyl-[4,4′-bipyridine]-3- carboxamide | 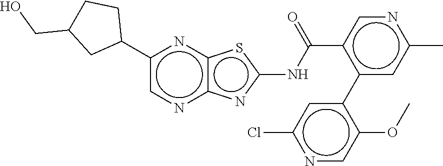 |
| 5 | (Racemic)-2′-chloro-5′-methoxy-6- methyl-N-(6-(tetrahydrofuran-3- yl)thiazolo[4,5-b]pyridin-2-yl)-[4,4′- bipyridine]-3-carboxamide | 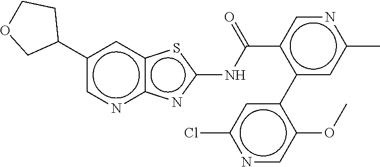 |
| 6 | (Racemic)-ethyl 3-(2-{2′-chloro-5′- methoxy-6-methyl-[4,4′-bipyridine]-3- amido}-[1,3]thiazolo[4,5-b]pyrazin-6- yl)cyclopentane-1-carboxylate | 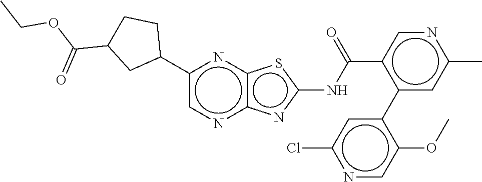 |
| 7 | (Racemic)-2′-chloro-N-(6-(5- (hydroxymethyl)tetrahydrofuran-3- yl)thiazolo[4,5-b]pyrazin-2-yl)-5′- methoxy-6-methyl-[4,4′-bipyridine]-3- carboxamide | 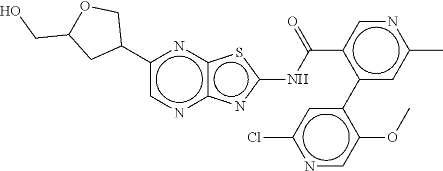 |
| 8 | 2′-chloro-N-(6-((1R or S,3S and R OR 1R and S,3S or R)-3- hydroxycyclopentyl)thiazolo[4,5- b]pyrazin-2-yl)-5′-methoxy-6-methyl- [4,4′-bipyridine]-3-carboxamide | 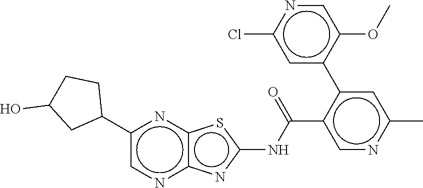 |
| 9 | 2′-chloro-N-(6-((1R and S,3S or R OR 1R or S,3S and R)-3- hydroxycyclopentyl)thiazolo[4,5- b]pyrazin-2-yl)-5′-methoxy-6-methyl- [4,4′-bipyridine]-3-carboxamide | 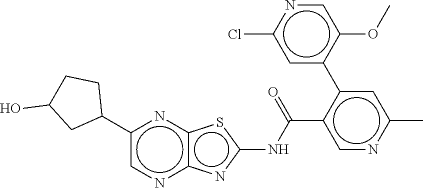 |
| 10 | 2′-chloro-N-(6- (cyclobutanecarboxamido)thiazolo[4,5- b]pyrazin-2-yl)-5′-methoxy-6- methyl-[4,4′-bipyridine]-3- carboxamide | 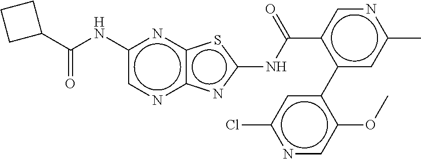 |
| 11 | 2′-chloro-5′-methoxy-6-methyl-N-(6- (2-oxopiperidin-1-yl)thiazolo[4,5- b]pyridin-2-yl)-[4,4′-bipyridine]-3- carboxamide | 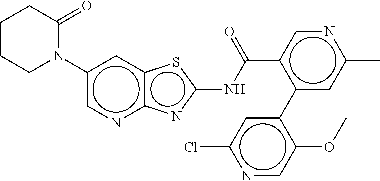 |
| 12 | 2-(2′-chloro-5′-methoxy-6-methyl- [4,4′-bipyridine]-3-carboxamido)-N- cyclopropylthiazolo[4,5-b]pyrazine-6- carboxamide | 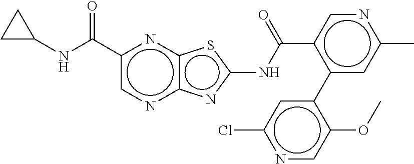 |
| 13 | (Racemic)-2′-chloro-N-(6-(3- (hydroxymethyl)pyrrolidin-1- yl)thiazolo[4,5-b]pyrazin-2-yl)-5′- methoxy-6-methyl-[4,4′-bipyridine]-3- carboxamide | 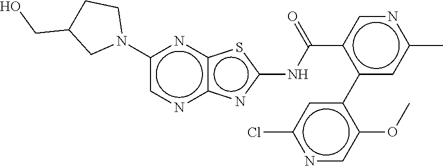 |
| 14 | 6-methyl-4-(pyrazolo[1,5-a]pyridin-4- yl)-N-(6-(pyridin-4-yl)thiazolo[4,5- b]pyrazin-2-yl)nicotinamide | 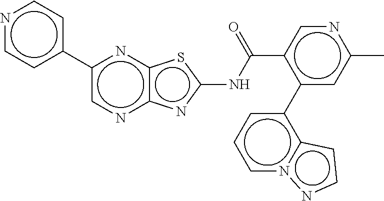 |
| 15 | (Racemic)-2′-chloro-N-(6-(3- hydroxypyrrolidin-1-yl)thiazolo[4,5- b]pyrazin-2-yl)-5′-methoxy-6-methyl- [4,4′-bipyridine]-3-carboxamide | 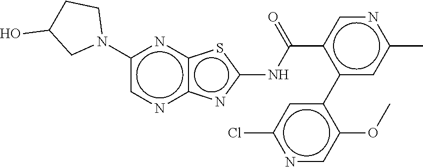 |
| 16 | 6-(azetidin-3-yl)-4-(2- methoxyphenyl)-N-(6-(pyridin-4- yl)thiazolo[4,5-b]pyrazin-2- yl)nicotinamide | 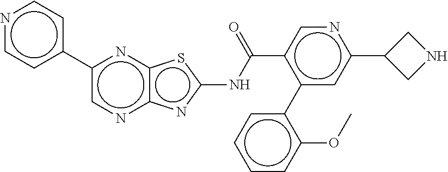 |
| 17 | 4-(7-methoxy-1-oxo-1,2,3,4- tetrahydroisoquinolin-6-yl)-6-methyl- N-(6-(pyridin-4-yl)thiazolo[4,5- b]pyrazin-2-yl)nicotinamide | 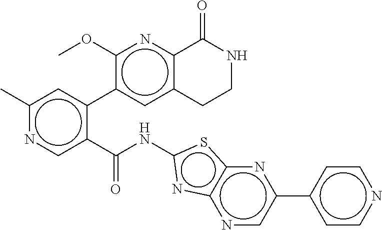 |
| 18 | 4-(5-cyano-2-methoxyphenyl)-N-(6- (cyclobutanecarboxamido)thiazolo[4,5- b]pyrazin-2-yl)-6- methylnicotinamide | 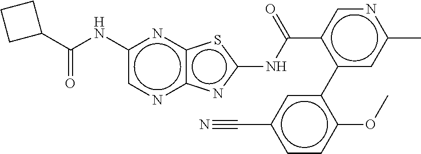 |
| 19 | 4-(5-cyano-2-methoxyphenyl)-N-(6- (3-(dimethylamino)azetidine-1- carbonyl)thiazolo[4,5-b]pyrazin-2-yl)- 6-methylnicotinamide | 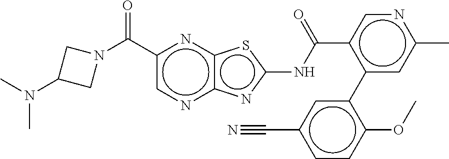 |
| 20 | 4-(5-cyano-2-methoxyphenyl)-6- methyl-N-(6-(2-oxopiperidin-1- yl)thiazolo[4,5-b]pyrazin-2- yl)nicotinamide | 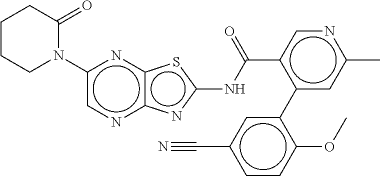 |
| 21 | 4-(5-cyano-2-methoxyphenyl)-6- methyl-N-(6-(2-oxopyrrolidin-1- yl)thiazolo[4,5-b]pyrazin-2- yl)nicotinamide | 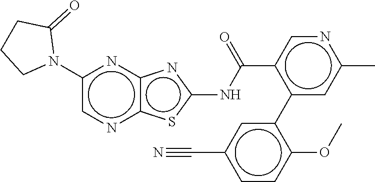 |
| 22 | (Racemic)-4-(5-cyano-2- methoxyphenyl)-6-methyl-N-(6- (tetrahydro-2H-pyran-3- yl)thiazolo[4,5-b]pyrazin-2- yl)nicotinamide | 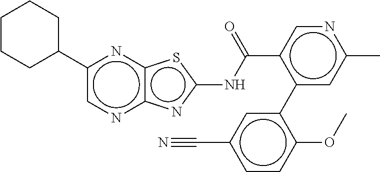 |
| 23 | 2′-chloro-5′-methoxy-6-methyl-N-(6- (2-methylpyridin-4-yl)thiazolo[4,5- b]pyridin-2-yl)-[4,4′-bipyridine]-3- carboxamide | 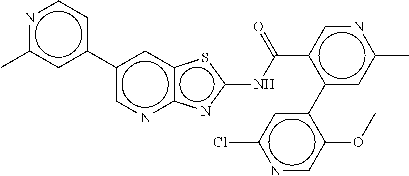 |
| 24 | 2′-chloro-5′-methoxy-6-methyl-N-[6- (2-methylpyridin-4-yl)- [1,3]thiazolo[4,5-b]pyrazin-2-yl]-[4,4′- bipyridine]-3-carboxamide | 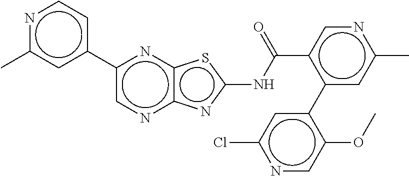 |
| 25 | 4-(2-methoxyphenyl)-6-methyl-N-(6- (pyridin-4-yl)thiazolo[4,5-b]pyrazin- 2-yl)nicotinamide | 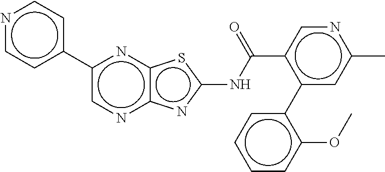 |
| 26 | (S)-4-(5-cyano-2-methoxyphenyl)-6- methyl-N-(6-(2-methylazetidine-1- carbonyl)thiazolo[4,5-b]pyrazin-2- yl)nicotinamide | 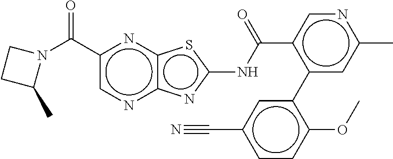 |
| 27 | N-(6-(1- cyanocyclopropyl)thiazolo[4,5- b]pyrazin-2-yl)-4-(2-methoxyphenyl)- 6-methylnicotinamide | 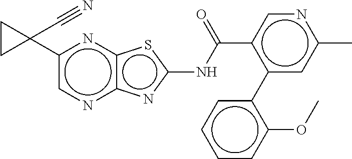 |
| 28 | (R)-4-(5-cyano-2-methoxyphenyl)-N- (6-(3-(dimethylamino)pyrrolidine-1- carbonyl)thiazolo[4,5-b]pyrazin-2-yl)- 6-methylnicotinamide | 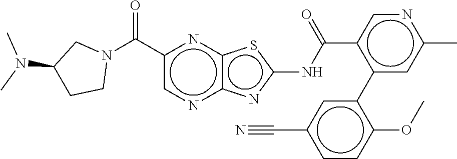 |
| 29 | 4-(5-cyano-2-methoxyphenyl)-6- methyl-N-(6-(3- oxomorpholino)thiazolo[4,5- b]pyrazin-2-yl)nicotinamide | 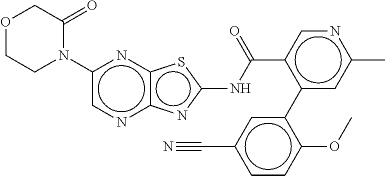 |
| 30 | N-(6-((1r,4r)-4- hydroxycyclohexyl)thiazolo[4,5- b]pyrazin-2-yl)-4-(2-methoxyphenyl)- 6-methylnicotinamide | 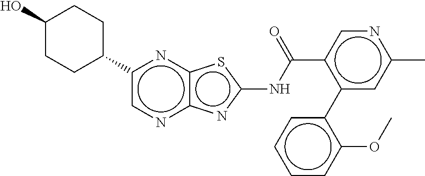 |
| 31 | N-(6-((1s,4s)-4- hydroxycyclohexyl)thiazolo[4,5- b]pyrazin-2-yl)-4-(2-methoxyphenyl)- 6-methylnicotinamide | 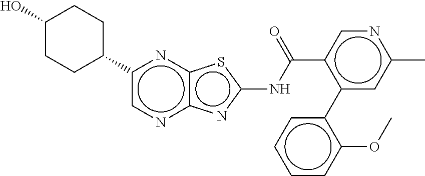 |
| 32 | 4-bromo-6-methyl-N-(6-(pyridin-4- yl)thiazolo[4,5-b]pyrazin-2- yl)nicotinamide | 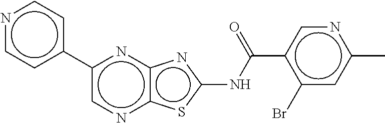 |
| 33 | 2-(4-(5-cyano-2-methoxyphenyl)-6- methylnicotinamido)-N,N- dimethylthiazolo[4,5-b]pyrazine-6- carboxamide | 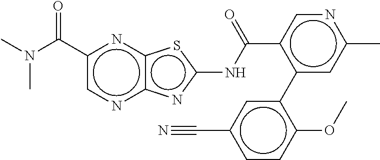 |
| 34 | (S)-4-(5-cyano-2-methoxyphenyl)-N- (6-(3-(dimethylamino)pyrrolidine-1- carbonyl)thiazolo[4,5-b]pyrazin-2-yl)- 6-methylnicotinamide | 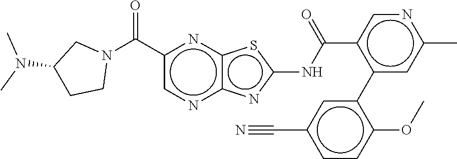 |
| 35 | 4-(5-cyano-2-methoxyphenyl)-6- methyl-N-(6-(piperazine-1- carbonyl)thiazolo[4,5-b]pyrazin-2- yl)nicotinamide | 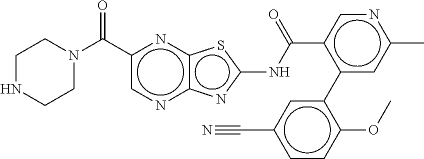 |
| 36 | tert-butyl 4-(2-(4-(5-cyano-2- methoxyphenyl)-6- methylnicotinamido)thiazolo[4,5- b]pyrazine-6-carbonyl)piperazine-1- carboxylate | 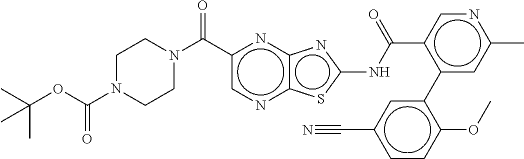 |
| 37 | 4-(5-cyano-2-methoxyphenyl)-6- methyl-N-(6-(pyrrolidine-1- carbonyl)thiazolo[4,5-b]pyrazin-2- yl)nicotinamide | 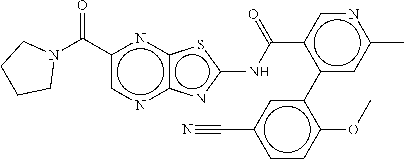 |
| 38 | (Cis and Trans)-N-(6-(4- hydroxycyclohexyl)thiazolo[4,5- b]pyrazin-2-yl)-4-(2-methoxyphenyl)- 6-methylnicotinamide | 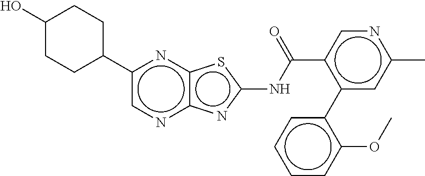 |
| 39 | (Racemic)-N-(6-((1,4-Dioxan-2- yl)methyl)thiazolo[4,5-b]pyrazin-2- yl)-4-(2-methoxyphenyl)-6- methylnicotinamide | 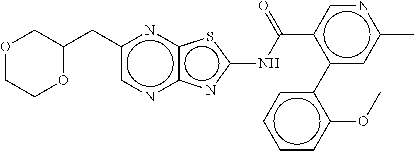 |
| 40 | 4-(5-cyano-2-methoxyphenyl)-6- methyl-N-(6-(3-methylazetidine-1- carbonyl)thiazolo[4,5-b]pyrazin-2- yl)nicotinamide | 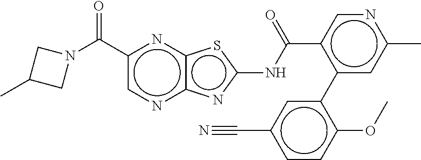 |
| 41 | (R)-4-(5-cyano-2-methoxyphenyl)-6- methyl-N-(6-(2-methylazetidine-1- carbonyl)thiazolo[4,5-b]pyrazin-2- yl)nicotinamide | 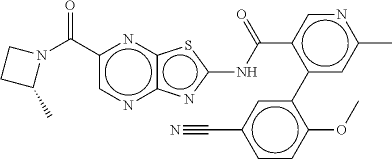 |
| 42 | 2-(4-(5-cyano-2-methoxyphenyl)-6- methylnicotinamido)-N- cyclopropylthiazolo[4,5-b]pyrazine-6- carboxamide | 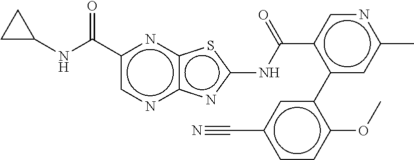 |
| 43 | 2-(4-(5-cyano-2-methoxyphenyl)-6- methylnicotinamido)-N- methylthiazolo[4,5-b]pyrazine-6- carboxamide | 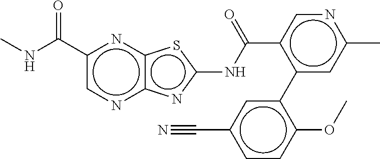 |
| 44 | 4-(5-cyano-2-methoxyphenyl)-N-(6- (3-methoxyazetidine-1- carbonyl)thiazolo[4,5-b]pyrazin-2-yl)- 6-methylnicotinamide | 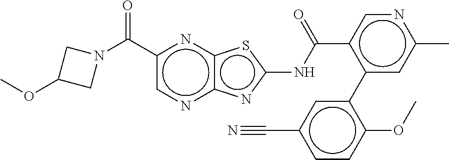 |
| 45 | 4-(5-cyano-2-methoxyphenyl)-6- methyl-N-(6-(tetrahydro-2H-pyran-4- yl)thiazolo[4,5-b]pyrazin-2- yl)nicotinamide | 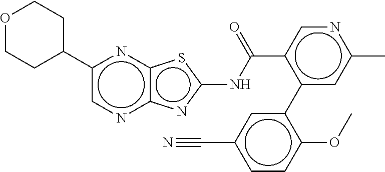 |
| 46 | 4-(6-methoxyimidazo[1,5-a]pyridin-7- yl)-6-methyl-N-(thiazolo[4,5- d]pyrimidin-2-yl)nicotinamide | 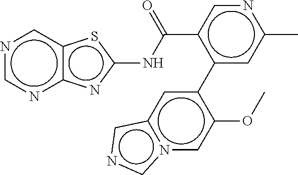 |
| 47 | 4-(5-cyano-2-methoxyphenyl)-6- methyl-N-(6-(morpholine-4- carbonyl)thiazolo[4,5-b]pyrazin-2- yl)nicotinamide | 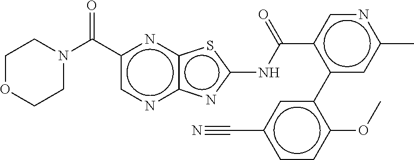 |
| 48 | 4-(5-cyano-2-methoxyphenyl)-6- methyl-N-(6-(piperidine-1- carbonyl)thiazolo[4,5-b]pyrazin-2- yl)nicotinamide | 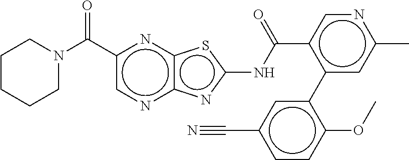 |
| 49 | N-(6-(5-(hydroxymethyl)pyridin-2- yl)thiazolo[4,5-b]pyrazin-2-yl)-4-(2- methoxyphenyl)-6- methylnicotinamide | 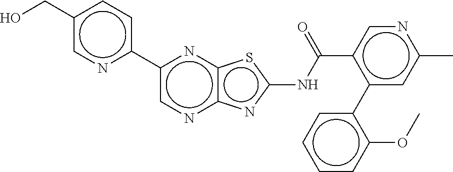 |
| 50 | 4-(5-cyano-2-methoxyphenyl)-6- methyl-N-{6-[(3S or R)-oxolan-3-yl]- [1,3]thiazolo[4,5-b]pyrazin-2- yl} pyridine-3-carboxamide | 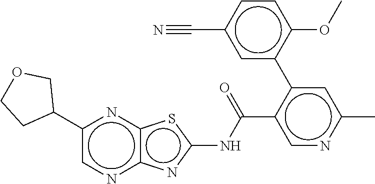 |
| 51 | 4-(5-cyano-2-methoxyphenyl)-6- methyl-N-{6-[(3R or S)-oxolan-3-yl]- [1,3]thiazolo[4,5-b]pyrazin-2- yl}pyridine-3-carboxamide | 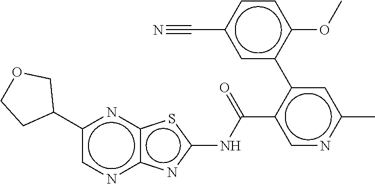 |
| 52 | 4-(5-cyano-2-methoxyphenyl)-6- methyl-N-(6-phenylthiazolo[5,4- c]pyridin-2-yl)nicotinamide | 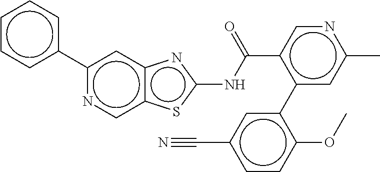 |
| 53 | (Racemic)-4-(2-methoxyphenyl)-6- methyl-N-(6-((tetrahydro-2H-pyran-2- yl)methyl)thiazolo[4,5-b]pyrazin-2- yl)nicotinamide | 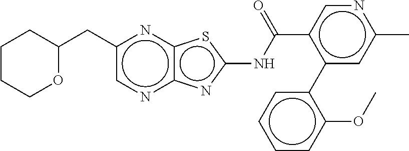 |
| 54 | (Racemic)-4-(2-methoxyphenyl)-6- methyl-N-(6-(tetrahydrofuran-2- yl)thiazolo[4,5-b]pyridin-2- yl)nicotinamide | 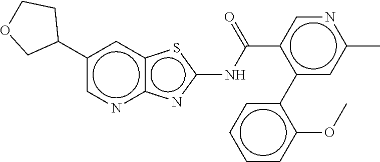 |
| 55 | 4-(2-(4-(2-methoxyphenyl)-6- methylnicotinamido)thiazolo[4,5- b]pyrazin-6-yl)pyridine 1-oxide | 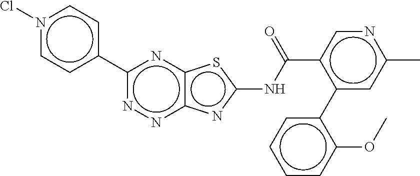 |
| 56 | 4-(5-cyano-2-methoxyphenyl)-6- methyl-N-(5-oxo-7-(pyridin-4-yl)-5H- [1,3,4]thiadiazolo[3,2-a]pyrimidin-2- yl)nicotinamide | 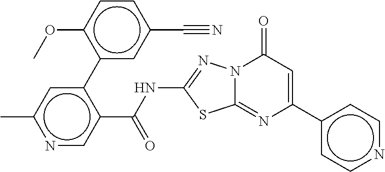 |
| 57 | 4-(5-cyano-2-methoxyphenyl)-6- methyl-N-(6-(pyridin-3- yl)thiazolo[5,4-c]pyridin-2- yl)nicotinamide | 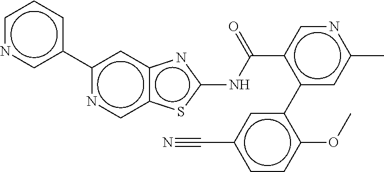 |
| 58 | 4-(5-cyano-2-methoxyphenyl)-6- methyl-N-(7-methyl-5-oxo-5H- [1,3,4]thiadiazolo[3,2-a]pyrimidin-2- yl)nicotinamide | 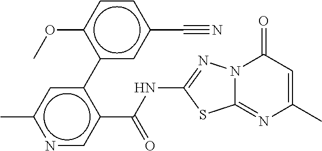 |
| 59 | 4-(5-cyano-2-methoxyphenyl)-6- methyl-N-(6-(2-methylpyridin-4- yl)thiazolo[4,5-b]pyrazin-2- yl)nicotinamide | 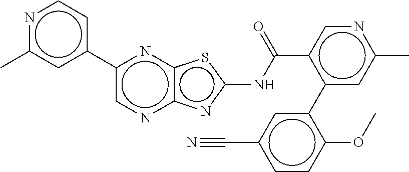 |
| 60 | N-(6-(4-chlorophenyl)thiazolo[4,5- b]pyridin-2-yl)-4-(6- methoxyimidazo[1,5-a]pyridin-7-yl)- 6-methylnicotinamide | 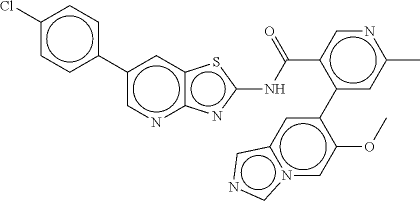 |
| 61 | N-(6-(4-chlorophenyl)thiazolo[4,5- b]pyrazin-2-yl)-4-(5-cyano-2- methoxyphenyl)-6- methylnicotinamide | 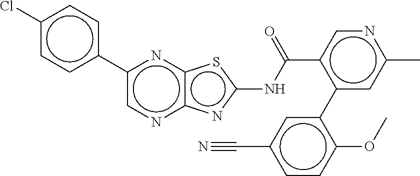 |
| 62 | N-(6-(4-chlorophenyl)thiazolo[4,5- b]pyridin-2-yl)-4-(5-cyano-2- methoxyphenyl)-6- methylnicotinamide | 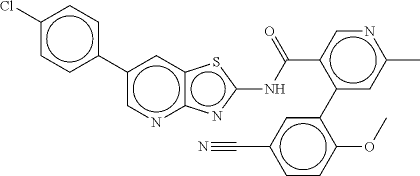 |
| 63 | 4-(5-cyano-2-methoxyphenyl)-6- methyl-N-(6-(pyridin-4- yl)thiazolo[4,5-b]pyridin-2- yl)nicotinamide | 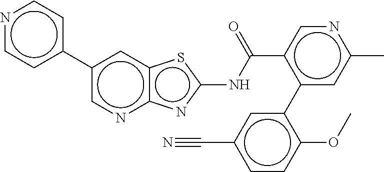 |
| 64 | 2′-chloro-N-(6-(4- chlorophenyl)thiazolo[4,5-b]pyrazin- 2-yl)-5′-methoxy-6-methyl-[4,4′- bipyridine]-3-carboxamide | 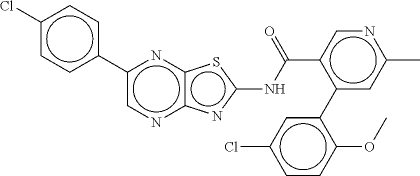 |
| 65 | 2′-chloro-5′-methoxy-6-methyl-N-(6- (pyridin-4-yl)thiazolo[4,5-b]pyrazin- 2-yl)-[4,4′-bipyridine]-3-carboxamide | 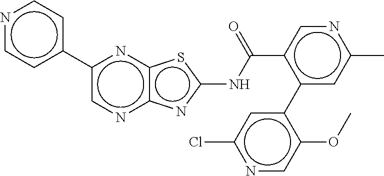 |
| 66 | 2′-chloro-5′-methoxy-6-methyl-N-(6- (pyridin-4-yl)thiazolo[4,5-b]pyridin-2- yl)-[4,4′-bipyridine]-3-carboxamide | 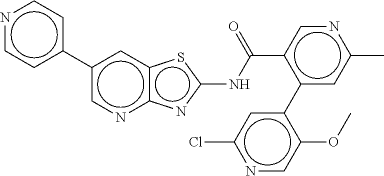 |
| 67 | 4-(5-cyano-2-methoxyphenyl)-6- methyl-N-(6-(pyridin-4- yl)thiazolo[4,5-b]pyrazin-2- yl)nicotinamide | 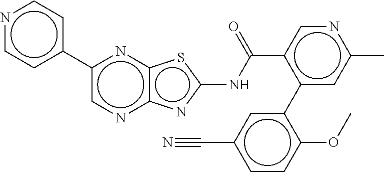 |
| 68 | 4-(5-cyano-2-methoxyphenyl)-6- methyl-N-(6-(2-methylpyridin-4- yl)thiazolo[4,5-b]pyridin-2- yl)nicotinamide | 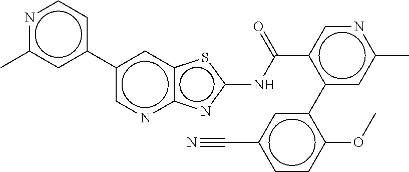 |
| 69 | N-(6-(4-chlorophenyl)thiazolo[4,5- b]pyrazin-2-yl)-4-(6- methoxyimidazo[1,5-a]pyridin-7-yl)- 6-methylnicotinamide | 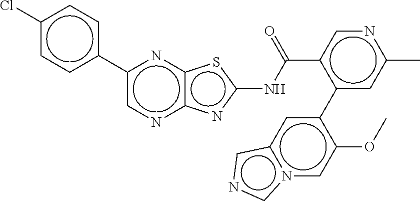 |
| 70 | N-(6-(4-chlorophenyl)thiazolo[4,5- b]pyridin-2-yl)-4-(6-methoxy-1- oxoisoindolin-5-yl)-6- methylnicotinamide | 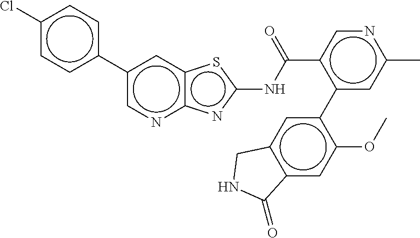 |
| 71 | 2′-cyano-5′-methoxy-6-methyl-N-(6- (pyridin-4-yl)thiazolo[4,5-b]pyrazin- 2-yl)-[4,4′-bipyridine]-3-carboxamide | 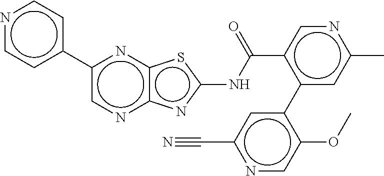 |
| 72 | N-(6-(4-(1- cyanocyclopropyl)phenyl)thiazolo[4,5- b]pyridin-2-yl)-4-(2-methoxyphenyl)- 6-methylnicotinamide | 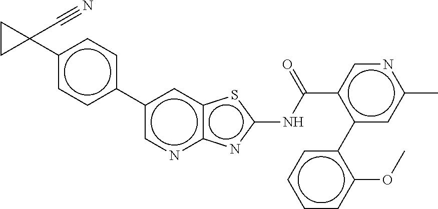 |
| 73 | 4-(6-methoxyimidazo[1,5-a]pyridin-7- yl)-6-methyl-N-(6-(pyridin-4- yl)thiazolo[4,5-b]pyrazin-2- yl)nicotinamide | 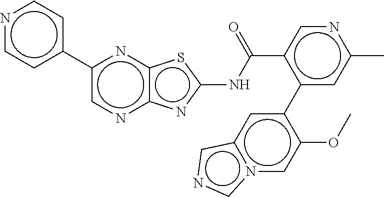 |
| 74 | 4-(2-methoxyphenyl)-6-methyl-N-(6- (2-methylpyridin-4-yl)thiazolo[4,5- b]pyrazin-2-yl)nicotinamide | 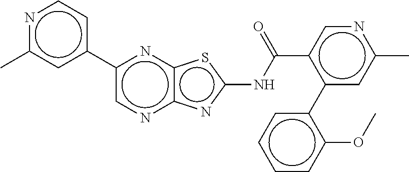 |
| 75 | N-(6-(4-chlorophenyl)thiazolo[4,5- b]pyrazin-2-yl)-4-(2-methoxyphenyl)- 6-methylnicotinamide | 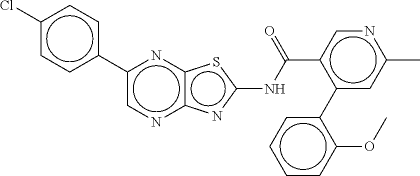 |
| 76 | N-(6-(4-chlorophenyl)thiazolo[4,5- b]pyridin-2-yl)-4-(2- (difluoromethoxy)phenyl)-6- methylnicotinamide | 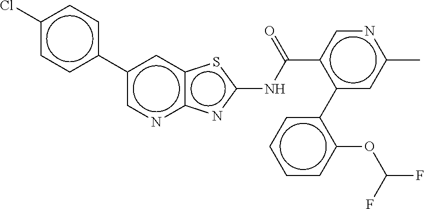 |
| 77 | N-(6-(4-chlorophenyl)thiazolo[4,5- c]pyridin-2-yl)-4-(5-cyano-2- methoxyphenyl)-6- methylnicotinamide | 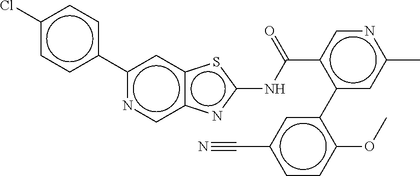 |
| 78 | N-(6-(5-(hydroxymethyl)pyridin-2- yl)thiazolo[4,5-b]pyridin-2-yl)-4-(2- methoxyphenyl)-6- methylnicotinamide | 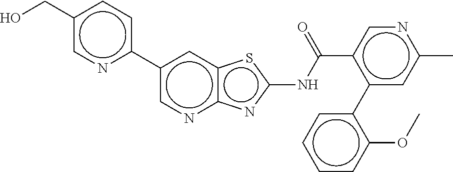 |
| 79 | 4-(6-methoxy-1-oxoisoindolin-5-yl)- 6-methyl-N-(6-(2-methylpyridin-4- yl)thiazolo[4,5-b]pyridin-2- yl)nicotinamide | 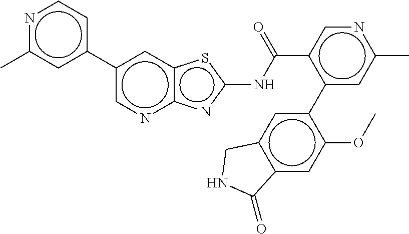 |
| 80 | 4-(2-methoxyphenyl)-6-methyl-N-(6- (3-methylpyridin-4-yl)thiazolo[4,5- b]pyridin-2-yl)nicotinamide | 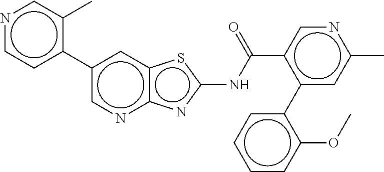 |
| 81 | 4-(6-methoxy-1-oxoisoindolin-5-yl)- 6-methyl-N-(6-(pyridin-4- yl)thiazolo[4,5-b]pyrazin-2- yl)nicotinamide | 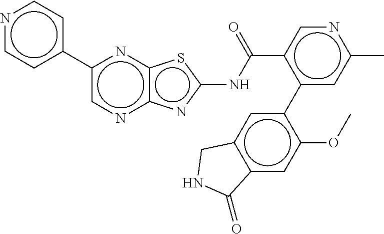 |
| 82 | N-(6-(4- cyclopropylphenyl)thiazolo[4,5- b]pyridin-2-yl)-4-(2-methoxyphenyl)- 6-methylnicotinamide | 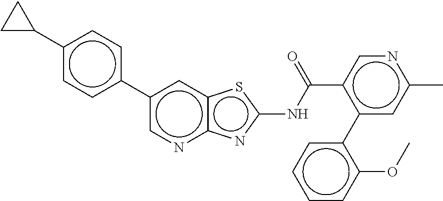 |
| 83 | 4-(2-methoxyphenyl)-6-methyl-N-(6- (pyridin-4-yl)thiazolo[4,5-b]pyridin-2- yl)nicotinamide | 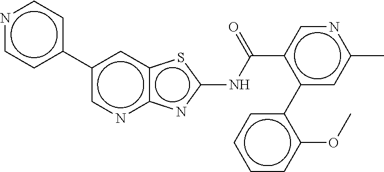 |
| 84 | N-(6-(4-chlorophenyl)thiazolo[4,5- c]pyridin-2-yl)-4-(6- methoxyimidazo[1,5-a]pyridin-7-yl)- 6-methylnicotinamide | 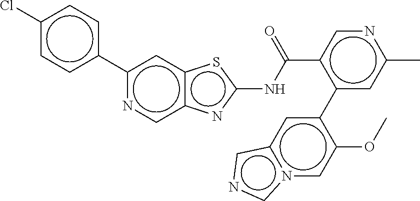 |
| 85 | N-(6-(4-chlorophenyl)thiazolo[4,5- b]pyridin-2-yl)-4-(2-methoxyphenyl)- 6-methylnicotinamide | 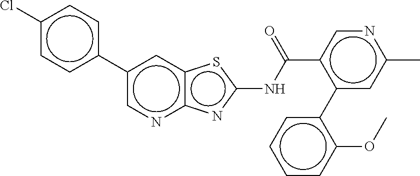 |
| 86 | N-(6-(azetidine-1- carbonyl)thiazolo[4,5-b]pyrazin-2-yl)- 4-(5-cyano-2-methoxyphenyl)-6- methylnicotinamide | 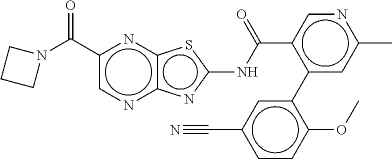 |
| 87 | 4-(2-methoxyphenyl)-6-methyl-N-(6- (2-methylpyridin-4-yl)thiazolo[4,5- b]pyridin-2-yl)nicotinamide | 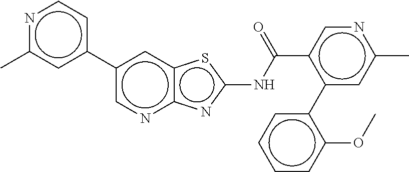 |
| 88 | N-(6-(4-chlorophenyl)thiazolo[4,5- b]pyridin-2-yl)-5-(2- methoxyphenyl)pyridazine-4- carboxamide | 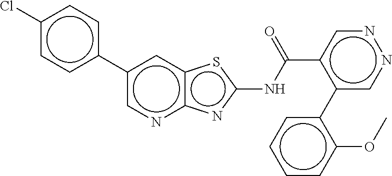 |
| 89 | 4-(2-methoxyphenyl)-6-methyl-N-(6- (2-oxopiperidin-1-yl)thiazolo[4,5- b]pyrazin-2-yl)nicotinamide | 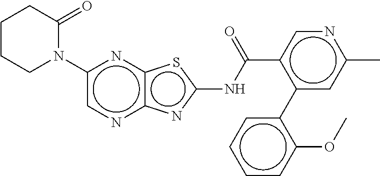 |
| 90 | 4-(6-methoxy-1-oxoisoindolin-5-yl)- 6-methyl-N-(6-(2-methylpyridin-4- yl)thiazolo[4,5-b]pyrazin-2- yl)nicotinamide | 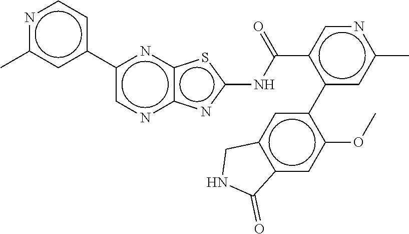 |
| 91 | N-(6-(4-chlorophenyl)thiazolo[4,5- b]pyridin-2-yl)-4-(2,4- dimethoxyphenyl)-6- methylnicotinamide | 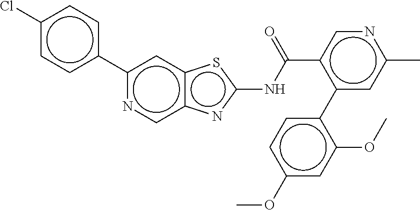 |
| 92 | 4-(2-methoxyphenyl)-6-methyl-N-(6- (3-methylpyridin-2-yl)thiazolo[4,5- b]pyridin-2-yl)nicotinamide | 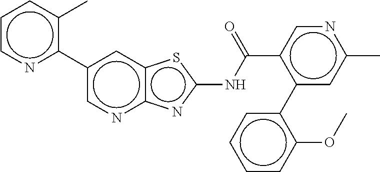 |
| 93 | N-(6-(3,4-dihydro-2H-pyran-6- yl)thiazolo[4,5-b]pyridin-2-yl)-4-(2- methoxyphenyl)-6- methylnicotinamide | 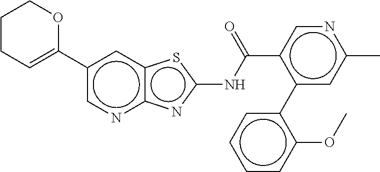 |
| 94 | (R or S)-4-(2-methoxyphenyl)-6- methyl-N-(6-(tetrahydro-2H-pyran-2- yl)thiazolo[4,5-b]pyridin-2- yl)nicotinamide | 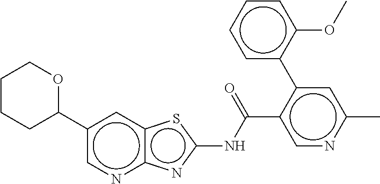 |
| 95 | N-(5-(4-chlorophenyl)thiazolo[5,4- d]pyrimidin-2-yl)-4-(2- methoxyphenyl)-6- methylnicotinamide | 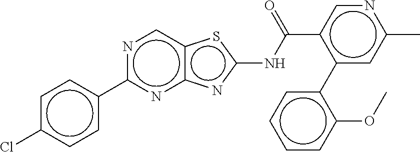 |
| 96 | N-(6-(4-chlorophenyl)thiazolo[4,5- c]pyridin-2-yl)-4-(2-methoxyphenyl)- 6-methylnicotinamide | 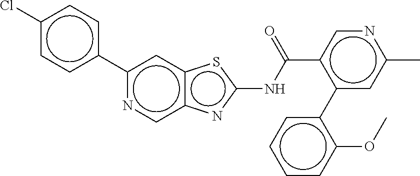 |
| 97 | 6-methyl-4-(1-methyl-1,4,6,7- tetrahydro-5H-pyrazolo[4,3-c]pyridin- 5-yl)-N-(6-(pyridin-4-yl)thiazolo[4,5- b]pyrazin-2-yl)nicotinamide | 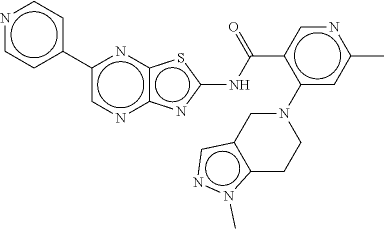 |
| 98 | N-(6-bromothiazolo[4,5-b]pyrazin-2- yl)-4-(2-methoxyphenyl)-6- methylnicotinamide | 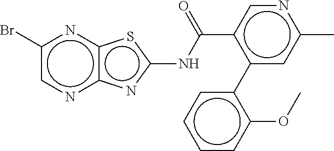 |
| 99 | 4-(2-methoxyphenyl)-2-methyl-N-(6- (pyridin-4-yl)thiazolo[4,5-b]pyrazin- 2-yl)pyrimidine-5-carboxamide | 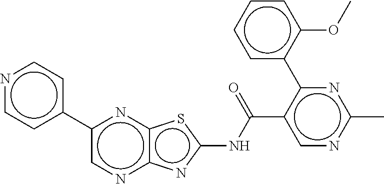 |
| 100 | N-(6-chlorothiazolo[4,5-c]pyridin-2- yl)-4-(2-methoxyphenyl)-6- methylnicotinamide | 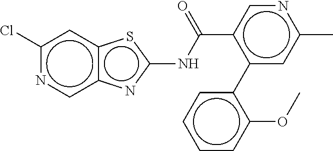 |
| 101 | (Racemic)-4-(2-methoxyphenyl)-6- methyl-N-(6-(tetrahydro-2H-pyran-2- yl)thiazolo[4,5-b]pyridin-2- yl)nicotinamide | 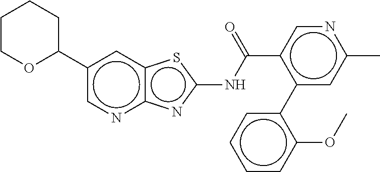 |
| 102 | tert-butyl 4-(2-methyl-5-((6-(pyridin- 4-yl)thiazolo[4,5-b]pyrazin-2- yl)carbamoyl)pyrimidin-4- yl)piperazine-1-carboxylate | 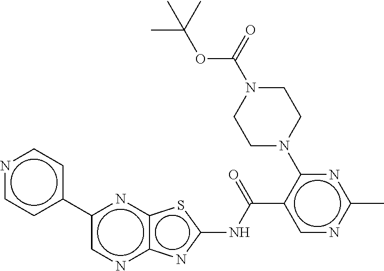 |
| 103 | N-(6-bromothiazolo[4,5-b]pyridin-2- yl)-4-(2,4-dimethoxyphenyl)-6- methylnicotinamide | 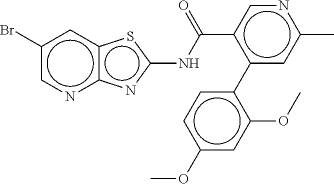 |
| 104 | 4-(2-methoxyphenyl)-6-methyl-N-(6- (pyridin-4-yl)benzo[d]thiazol-2- yl)nicotinamide | 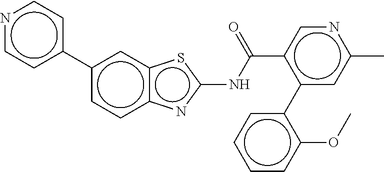 |
| 105 | N-(6-cyclopropylthiazolo[4,5- b]pyridin-2-yl)-4-(2-methoxyphenyl)- 6-methylnicotinamide | 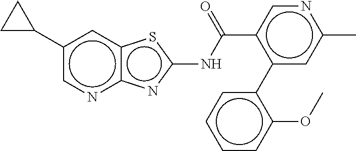 |
| 106 | (S or R)-4-(2-methoxyphenyl)-6- methyl-N-(6-(tetrahydro-2H-pyran-2- yl)thiazolo[4,5-b]pyridin-2- yl)nicotinamide | 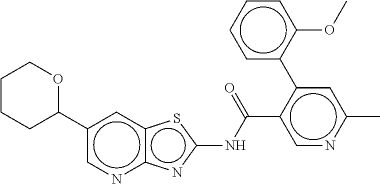 |
| 107 | N-(6-(4-chlorophenyl)thiazolo[4,5- c]pyridin-2-yl)-4-(2- (difluoromethoxy)phenyl)-6- methylnicotinamide | 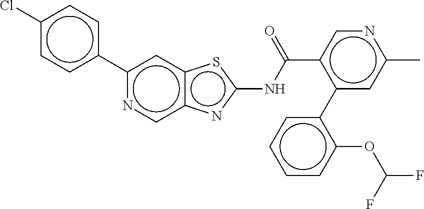 |
| 108 | tert-butyl 4-(2-cyano-5-((6-(pyridin-4- yl)thiazolo[4,5-b]pyrazin-2- yl)carbamoyl)pyridin-4-yl)piperazine- 1-carboxylate | 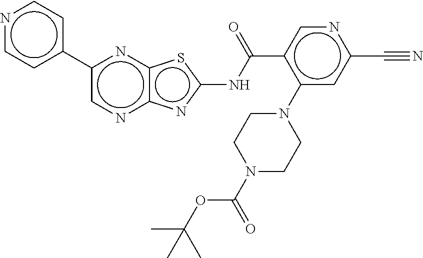 |
| 109 | N-(6-bromothiazolo[4,5-b]pyridin-2- yl)-4-(2-methoxyphenyl)-6- methylnicotinamide | 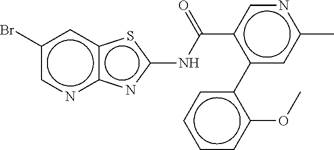 |
| 110 | N-(6-(imidazo[1,2-a]pyridin-6- yl)benzo[d]thiazol-2-yl)-4-(2- methoxyphenyl)-6- methylnicotinamide | 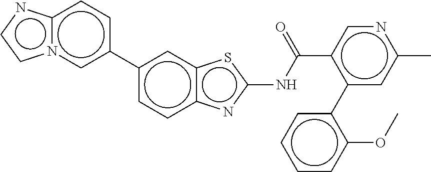 |
| 111 | N-(6-(imidazo[1,5-a]pyridin-6- yl)benzo[d]thiazol-2-yl)-4-(2- methoxyphenyl)-6- methylnicotinamide | 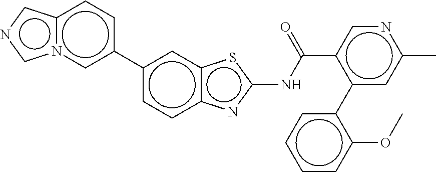 |
| 112 | (R)-4-(3-cyanopiperidin-1-yl)-6- methyl-N-(6-(pyridin-4- yl)thiazolo[4,5-b]pyrazin-2- yl)nicotinamide | 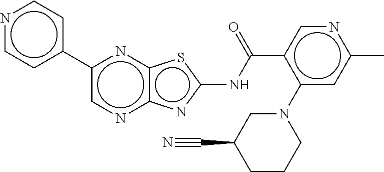 |
| 113 | 6-methyl-N-(6-(pyridin-4- yl)thiazolo[4,5-b]pyrazin-2-yl)-4- (1,4,6,7-tetrahydro-5H-pyrazolo[4,3- c]pyridin-5-yl)nicotinamide | 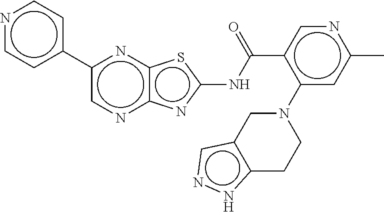 |
| 114 | 4-(2-methoxyphenyl)-6-methyl-N-(6- (2-oxopiperidin-1-yl)benzo[d]thiazol- 2-yl)nicotinamide | 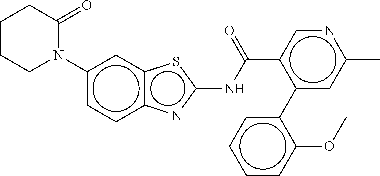 |
| 115 | N-(6-(4-chlorophenyl)thiazolo[4,5- b]pyridin-2-yl)-7-(2- methoxyphenyl)imidazo[1,2- alpyridine-6-carboxamide | 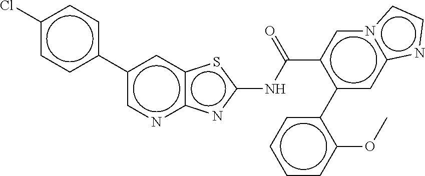 |
| 116 | 4-(2-methoxyphenyl)-6-methyl-N-(6- (trifluoromethyl)thiazolo[4,5- b]pyridin-2-yl)nicotinamide | 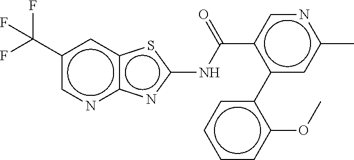 |
| 117 | N-(3-chlorothiazolo[4,5-c]pyridazin- 6-yl)-4-(2-methoxyphenyl)-6- methylnicotinamide | 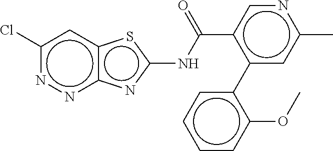 |
| 118 | N-(5-(4-chlorophenyl)thiazolo[5,4- b]pyridin-2-yl)-4-(2-methoxyphenyl)- 6-methylnicotinamide | 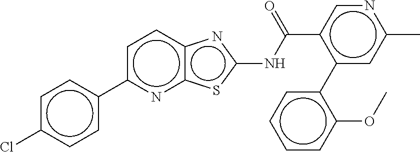 |
| 119 | 4-(6,7-dihydropyrazolo[1,5-a]pyrazin- 5(4H)-yl)-6-methyl-N-(6-(pyridin-4- yl)thiazolo[4,5-b]pyrazin-2- yl)nicotinamide | 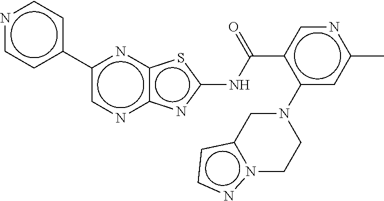 |
| 120 | N-(6-(3-cyanopyridin-2- yl)thiazolo[4,5-b]pyridin-2-yl)-4-(2- methoxyphenyl)-6- methylnicotinamide | 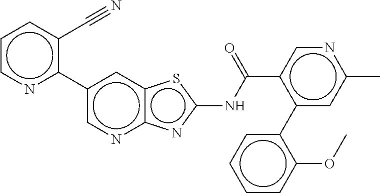 |
| 121 | 4-(2-methoxyphenyl)-6-methyl-N-(6- (pyridin-2-yl)benzo[d]thiazol-2- yl)nicotinamide | 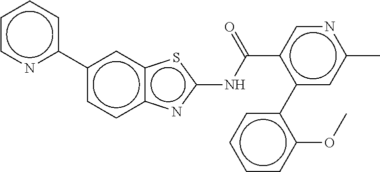 |
| 122 | 4-(2-methoxyphenyl)-6-methyl-N- (thiazolo[5,4-c]pyridin-2- yl)nicotinamide | 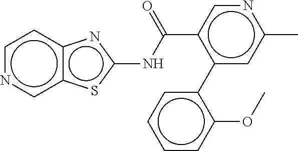 |
| 123 | tert-butyl 4-(2-methyl-5-((6-(pyridin- 4-yl)thiazolo[4,5-b]pyrazin-2- yl)carbamoyl)pyridin-4-yl)piperazine- 1-carboxylate | 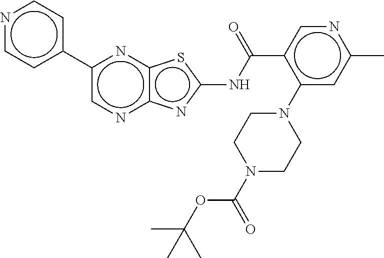 |
| 124 | 4-(5,6-dihydroimidazo[1,2-a]pyrazin- 7(8H)-yl)-6-methyl-N-(6-(pyridin-4- yl)thiazolo[4,5-b]pyrazin-2- yl)nicotinamide | 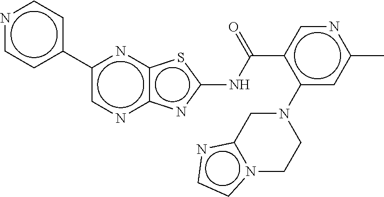 |
| 125 | 4-(2-methoxyphenyl)-2-methyl-N-(6- (pyridin-4-yl)thiazolo[4,5-b]pyridin-2- yl)pyrimidine-5-carboxamide | 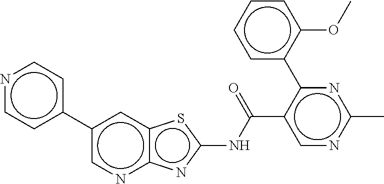 |
| 126 | N-(3-(4-chlorophenyl)thiazolo[4,5- c]pyridazin-6-yl)-4-(2- methoxyphenyl)-6- methylnicotinamide | 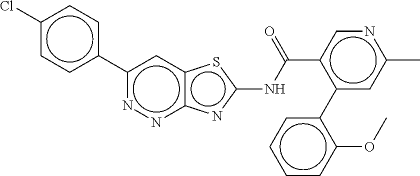 |
| 127 | 6-methyl-4-(2-methyl-2,4,6,7- tetrahydro-5H-pyrazolo[4,3-c]pyridin- 5-yl)-N-(6-(pyridin-4-yl)thiazolo[4,5- b]pyrazin-2-yl)nicotinamide | 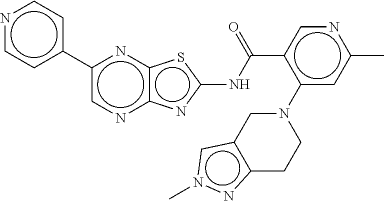 |
| 128 | (R)-N-(6-(4- chlorophenyl)thiazolo[4,5-b]pyridin- 2-yl)-6-methyl-4-(6- oxohexahydropyrrolo[1,2-a]pyrazin- 2(1H)-yl)nicotinamide | 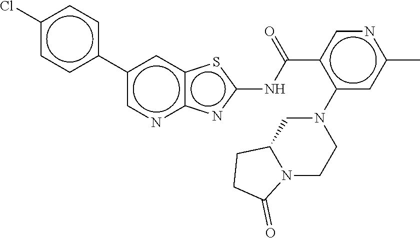 |
| 129 | 6-methyl-N-(6-(pyridin-4- yl)thiazolo[4,5-b]pyrazin-2-yl)-4-(4- oxa-7-azaspiro[2.5]octan-7- yl)nicotinamide | 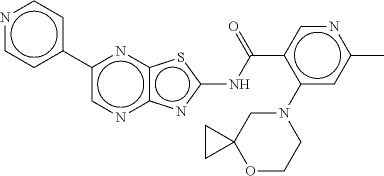 |
| 130 | N-(6-(4-chlorophenyl)thiazolo[4,5- b]pyridin-2-yl)-2′-methoxy-5-methyl- [1,1′-biphenyl]-2-carboxamide | 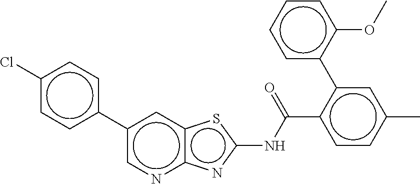 |
| 131 | 4-(2-methoxyphenyl)-6-methyl-N-(6- (pyridin-3-yl)benzo[d]thiazol-2- yl)nicotinamide | 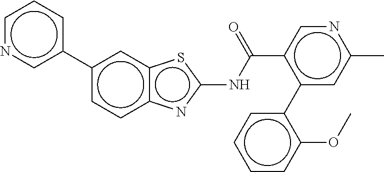 |
| 132 | N-(6-(4- chlorophenyl)benzo[d]thiazol-2-yl)-4- (2-methoxyphenyl)-6- methylnicotinamide | 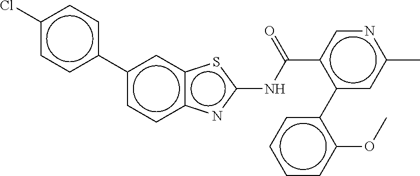 |
| 133 | N-(6-(4-chlorophenyl)thiazolo[4,5- b]pyridin-2-yl)-4-(2-methoxyphenyl)- 6-(trifluoromethyl)nicotinamide | 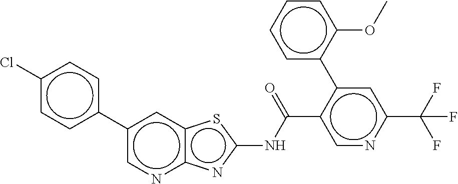 |
| 134 | N-(6-(4- cyclopropylphenyl)benzo[d]thiazol-2- yl)-4-(2-methoxyphenyl)-6- methylnicotinamide | 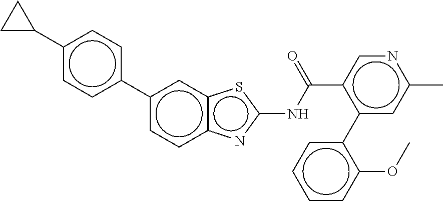 |
| 135 | N-(5-bromobenzo[d]thiazol-2-yl)-4- (2-methoxyphenyl)-6- methylnicotinamide | 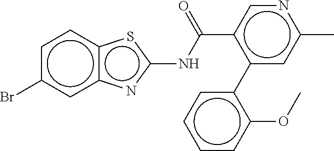 |
| 136 | tert-butyl 4-(2-cyano-5-((6-(pyridin-4- yl)thiazolo[4,5-b]pyridin-2- yl)carbamoyl)pyridin-4-yl)piperazine- 1-carboxylate | 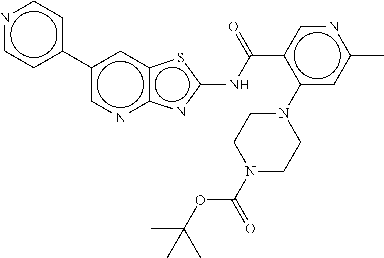 |
| 137 | 4-(2-methoxyphenyl)-6-methyl-N-(6- (piperidin-1-yl)thiazolo[4,5-c]pyridin- 2-yl)nicotinamide | 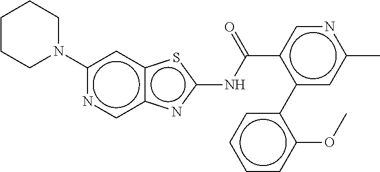 |
| 138 | N-(6-bromobenzo[d]thiazol-2-yl)-4- (2-methoxyphenyl)-6- methylnicotinamide | 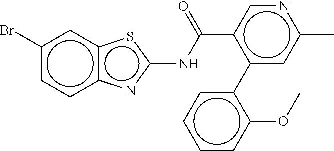 |
| 139 | tert-butyl 2′-methyl-5′-((6-(pyridin-4- yl)thiazolo[4,5-b]pyrazin-2- yl)carbamoyl)-3,6-dihydro-[4,4′- bipyridine]-1(2H)-carboxylate | 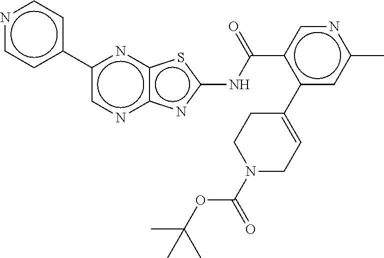 |
| 140 | (Racemic)-4-(2-methoxyphenyl)-6- methyl-N-(6-(tetrahydrofuran-2- yl)benzo[d]thiazol-2-yl)nicotinamide | 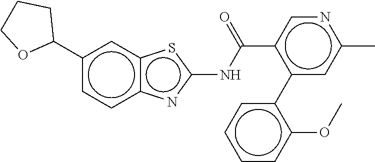 |
| 141 | tert-butyl 4-(2-methyl-5-((6-(pyridin- 4-yl)thiazolo[4,5-b]pyridin-2- yl)carbamoyl)pyridin-4-yl)piperazine- 1-carboxylate | 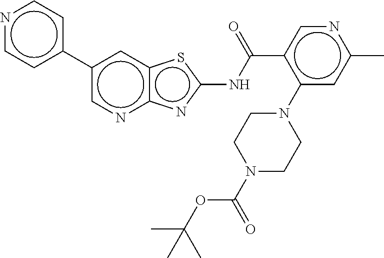 |
| 142 | 4-(2-methoxyphenyl)-6-methyl-N-(6- phenylbenzo[d]thiazol-2- yl)nicotinamide | 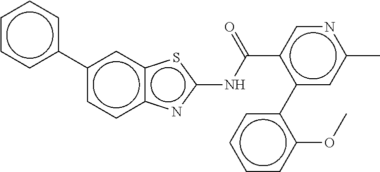 |
| 143 | (S)-4-(3-cyanopiperidin-1-yl)-6- methyl-N-(6-(pyridin-4- yl)thiazolo[4,5-b]pyridin-2- yl)nicotinamide | 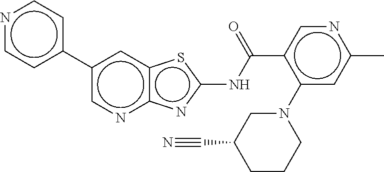 |
| 144 | (Racemic)-4-(2-methoxyphenyl)-6- methyl-N-(6-((tetrahydrofuran-2- yl)methyl)benzo[d]thiazol-2- yl)nicotinamide | 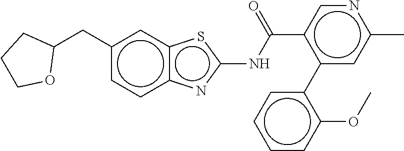 |
| 145 | N-(4-bromobenzo[d]thiazol-2-yl)-4- (2-methoxyphenyl)-6- methylnicotinamide | 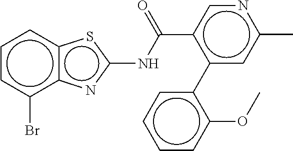 |
| 146 | (R)-4-(3-cyanopiperidin-1-yl)-6- methyl-N-(6-(pyridin-4- yl)thiazolo[4,5-b]pyridin-2- yl)nicotinamide | 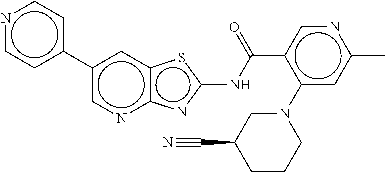 |
| 147 | tert-butyl 4-(2-methyl-5-((6-(pyridin- 4-yl)thiazolo[4,5-b]pyrazin-2- yl)carbamoyl)pyridin-4-yl)piperidine- 1-carboxylate | 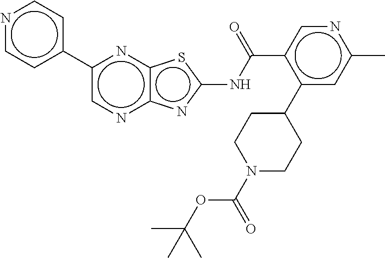 |
| 148 | tert-butyl 2′-methyl-5′-((6-(pyridin-4- yl)thiazolo[4,5-b]pyridin-2- yl)carbamoyl)-3,6-dihydro-[4,4′- bipyridine]-1(2H)-carboxylate | 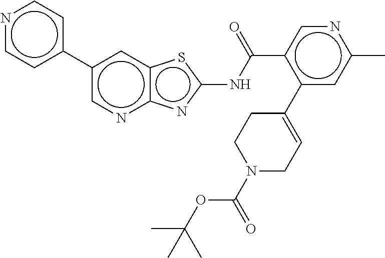 |
| 149 | (S)-N-(6-(5-(1- cyanocyclopropyl)pyridin-2- yl)thiazolo[4,5-b]pyridin-2-yl)-6- methyl-4-(3-oxotetrahydro-3H- oxazolo[3,4-a]pyrazin-7(1H)- yl)nicotinamide | 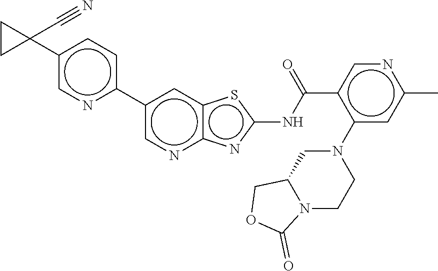 |
| 150 | N-(6-(3,4-dihydroisoquinolin-2(1H)- yl)benzo[d]thiazol-2-yl)-4-(2- methoxyphenyl)-6- methylnicotinamide | 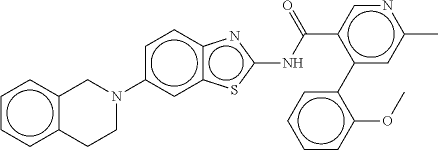 |
| 151 | (S)-4-(3-cyanopiperidin-1-yl)-6- methyl-N-(6-(pyridin-4- yl)thiazolo[4,5-b]pyrazin-2- yl)nicotinamide | 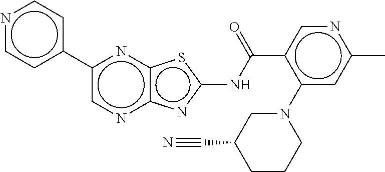 |
| 152 | 6-methyl-N-(6-(pyridin-4- yl)thiazolo[4,5-b]pyridin-2-yl)-4-(4- oxa-7-azaspiro[2.5]octan-7- yl)nicotinamide | 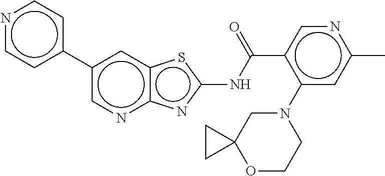 |
| 153 | 4-(5,6-dihydro-[1,2,4]triazolo[1,5- alpyrazin-7(8H)-yl)-6-methyl-N-(6- (pyridin-4-yl)thiazolo[4,5-b]pyrazin- 2-yl)nicotinamide | 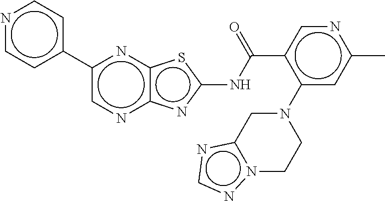 |
| 154 | 4-(2-methoxyphenyl)-6-methyl-N-(6- methylthiazolo[4,5-b]pyridin-2- yl)nicotinamide | 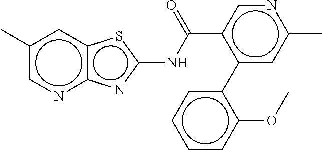 |
| 155 | 4-(2-methoxyphenyl)-6-methyl-N-(5- phenylbenzo[d]thiazol-2- yl)nicotinamide | 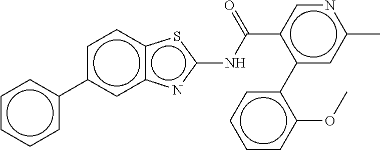 |
| 156 | N-(6-benzylbenzo[d]thiazol-2-yl)-4- (2-methoxyphenyl)-6- methylnicotinamide | 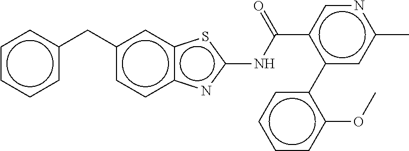 |
| 157 | 4-(2-methoxyphenyl)-6-methyl-N-(6- (piperidin-1-yl)benzo[d]thiazol-2- yl)nicotinamide | 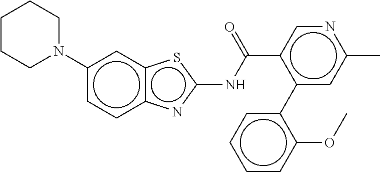 |
| 158 | (R)-6-methyl-4-(6- oxohexahydropyrrolo[1,2-a]pyrazin- 2(1H)-yl)-N-(6-(pyridin-4- yl)thiazolo[4,5-b]pyrazin-2- yl)nicotinamide | 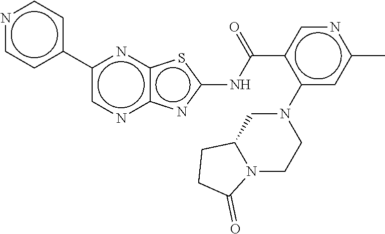 |
| 159 | 6-methyl-N-(6-(pyridin-4- yl)thiazolo[4,5-b]pyridin-2-yl)-4-(4- (thiazol-2-yl)piperazin-1- yl)nicotinamide | 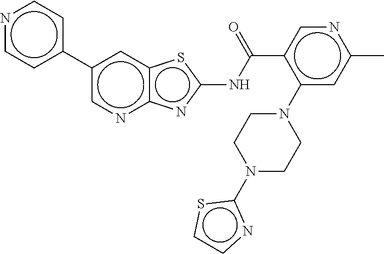 |
| 160 | tert-butyl 6-(2-methyl-5-((6-(pyridin- 4-yl)thiazolo[4,5-b]pyridin-2- yl)carbamoyl)pyridin-4-yl)-2,6- diazaspiro[3.3]heptane-2-carboxylate | 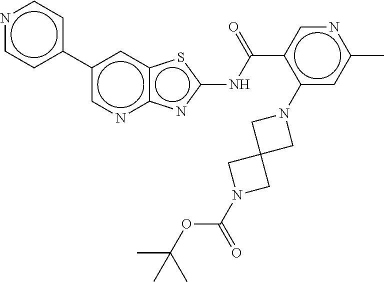 |
| 161 | N-(6-(cyclohex-1-en-1- yl)benzo[d]thiazol-2-yl)-4-(2- methoxyphenyl)-6- methylnicotinamide | 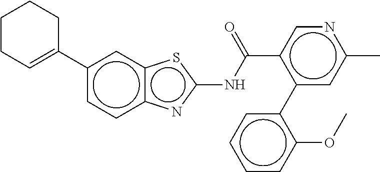 |
| 162 | 4-(2-methoxyphenyl)-6-methyl-N-(5- methylbenzo[d]thiazol-2- yl)nicotinamide | 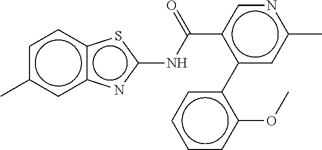 |
| 163 | 4-(2-methoxyphenyl)-6-methyl-N- (thiazolo[4,5-c]pyridin-2- yl)nicotinamide | 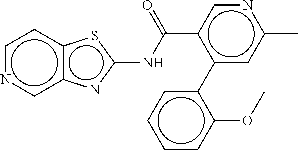 |
| 164 | 4-(2-methoxyphenyl)-6-methyl-N-(6- morpholinobenzo[d]thiazol-2- yl)nicotinamide | 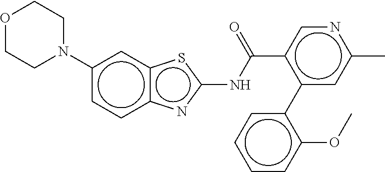 |
| 165 | (R)-N-(6-(5-(1- cyanocyclopropyl)pyridin-2- yl)thiazolo[4,5-b]pyridin-2-yl)-6- methyl-4-(3-oxotetrahydro-3H- oxazolo[3,4-a]pyrazin-7(1H)- yl)nicotinamide | 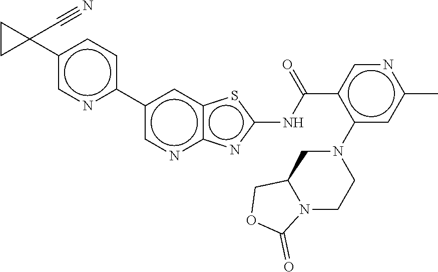 |
| 166 | tert-butyl 3-methyl-4-(2-methyl-5-((6- (pyridin-4-yl)thiazolo[4,5-b]pyridin-2- yl)carbamoyl)pyridin-4-yl)piperazine- 1-carboxylate | 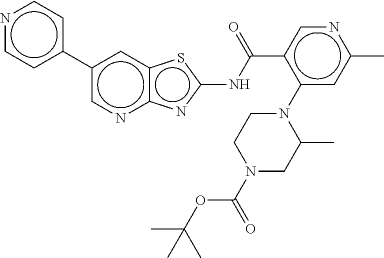 |
| 167 | N-(5-chlorothiazolo[5,4-b]pyridin-2- yl)-4-(2-methoxyphenyl)-6- methylnicotinamide | 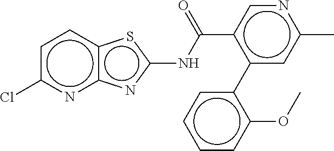 |
| 168 | (R or S)-6-methyl-4-(1-oxo-2,6- diazaspiro[3.5]nonan-6-yl)-N-(6- (pyridin-4-yl)thiazolo[4,5-b]pyrazin- 2-yl)nicotinamide | 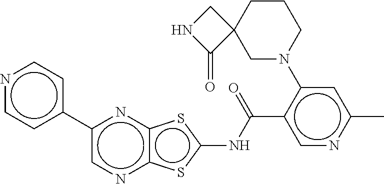 |
| 169 | (Racemic)-6-methyl-4-(2-oxo-1-oxa- 3,7-diazaspiro[4.5]decan-7-yl)-N-(6- (pyridin-4-yl)thiazolo[4,5-b]pyridin-2- yl)nicotinamide | 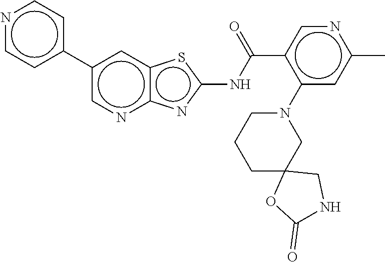 |
| 170 | 4-(4-(cyclopropylmethyl)-3- oxopiperazin-1-yl)-6-methyl-N-(6- (pyridin-4-yl)thiazolo[4,5-b]pyridin-2- yl)nicotinamide | 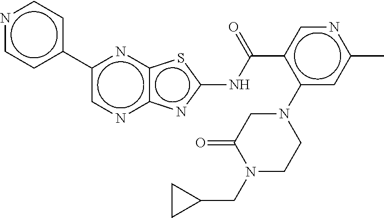 |
| 171 | 4-(5,6-dihydroimidazo[1,5-a]pyrazin- 7(8H)-yl)-6-methyl-N-(6-(pyridin-4- yl)thiazolo[4,5-b]pyrazin-2- yl)nicotinamide | 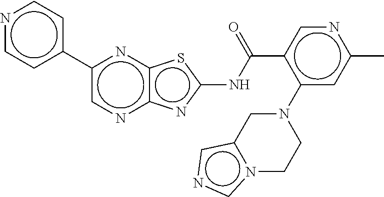 |
| 172 | 4-(2-methoxyphenyl)-6-methyl-N-(6- methylbenzo[d]thiazol-2- yl)nicotinamide | 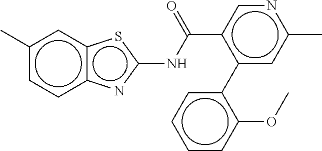 |
| 173 | (R or S)-6-methyl-4-(2-oxo-1,6- diazaspiro[3.5]nonan-6-yl)-N-(6- (pyridin-4-yl)thiazolo[4,5-b]pyrazin- 2-yl)nicotinamide | 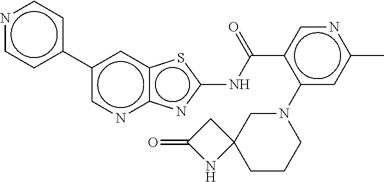 |
| 174 | 4-(2-methoxyphenyl)-6-methyl-N-(4- methylbenzo[d]thiazol-2- yl)nicotinamide | 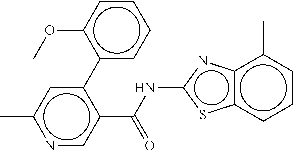 |
| 175 | tert-butyl 4-(2-methyl-5-((6-(pyridin- 4-yl)thiazolo[4,5-b]pyridin-2- yl)carbamoyl)pyridin-4-yl)piperidine- 1-carboxylate | 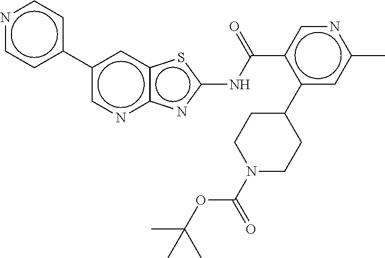 |
| 176 | N-(6-(2-oxa-6-azaspiro[3.3]heptan-6- yl)benzo[d]thiazol-2-yl)-4-(2- methoxyphenyl)-6- methylnicotinamide | 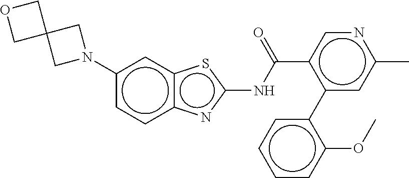 |
| 177 | N-(6-((1R,4R)-2-oxa-5- azabicyclo[2.2.1]heptan-5- yl)benzo[d]thiazol-2-yl)-4-(2- methoxyphenyl)-6- methylnicotinamide | 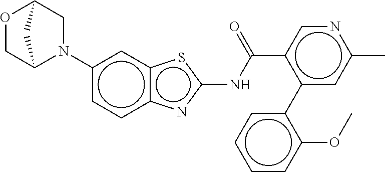 |
| 178 | 4-(4-cyclopropyl-3-oxopiperazin-1- yl)-6-methyl-N-(6-(pyridin-4- yl)thiazolo[4,5-b]pyridin-2- yl)nicotinamide | 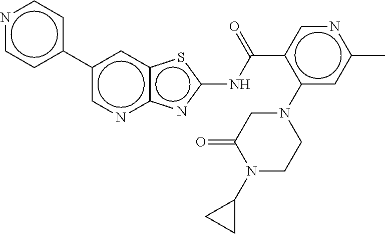 |
| 179 | N-(6-cyclohexylbenzo[d]thiazol-2-yl)- 4-(2-methoxyphenyl)-6- methylnicotinamide | 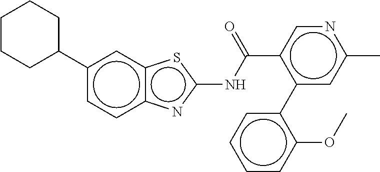 |
| 180 | N-(6-(4-chlorophenyl)thiazolo[4,5- b]pyridin-2-yl)-3-(2- methoxyphenyl)picolinamide | 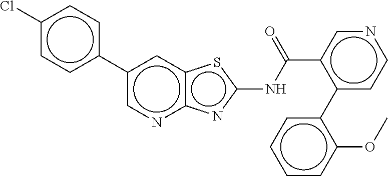 |
| 181 | (Racemic)-6-methyl-N-(6-(pyridin-4- yl)thiazolo[4,5-b]pyridin-2-yl)-4-(1- oxa-6-azaspiro[3.5]nonan-6- yl)nicotinamide | 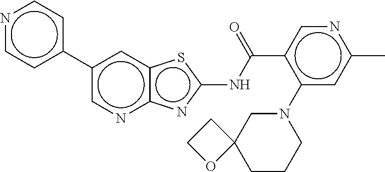 |
| 182 | N-(6-bromothiazolo[4,5-b]pyridin-2- yl)-6-methyl-4-(4-(thiazol-2- yl)piperazin-1-yl)nicotinamide | 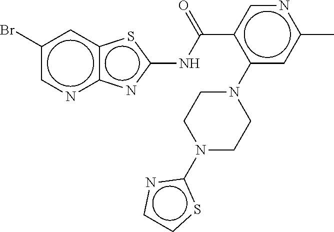 |
| 183 | 6-methyl-N-(6-(pyridin-4- yl)thiazolo[4,5-b]pyridin-2-yl)-4-(2- oxa-6-azaspiro[3.5]nonan-6- yl)nicotinamide | 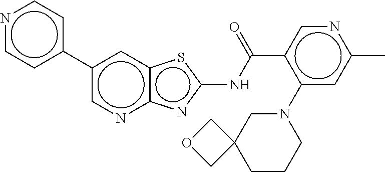 |
| 184 | (S or R)-6-methyl-4-(2-oxo-1,6- diazaspiro[3.5]nonan-6-yl)-N-(6- (pyridin-4-yl)thiazolo[4,5-b]pyrazin- 2-yl)nicotinamide | 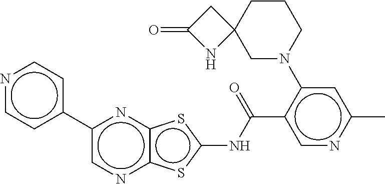 |
| 185 | (Racemic)-6-methyl-4-(1-oxo-2,6- diazaspiro[3.5]nonan-6-yl)-N-(6- (pyridin-4-yl)thiazolo[4,5-b]pyridin-2- yl)nicotinamide | 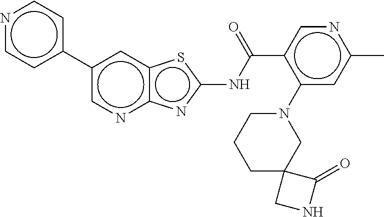 |
| 186 | N-(6-bromobenzo[d]oxazol-2-yl)-4- (2-methoxyphenyl)-6- methylnicotinamide | 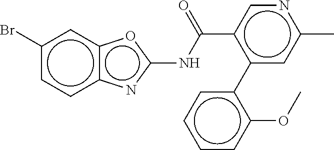 |
| 187 | 6-methyl-4-(1-oxo-2,7- diazaspiro[3.5]nonan-7-yl)-N-(6- (pyridin-4-yl)thiazolo[4,5-b]pyridin-2- yl)nicotinamide | 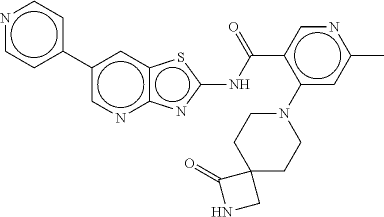 |
| 188 | N-(6-((1S,4S)-2-oxa-5- azabicyclo[2.2.1]heptan-5- yl)benzo[d]thiazol-2-yl)-4-(2- methoxyphenyl)-6- methylnicotinamide | 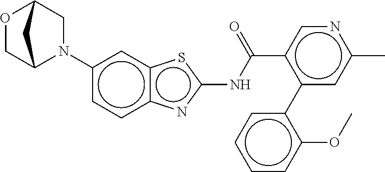 |
| 189 | (Racemic)-6-methyl-4-(2-oxo-1,6- diazaspiro[3.5]nonan-6-yl)-N-(6- (pyridin-4-yl)thiazolo[4,5-b]pyridin-2- yl)nicotinamide | 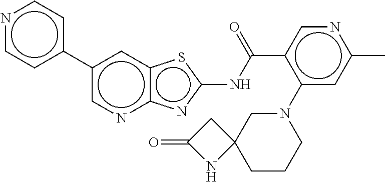 |
| 190 | 6-methyl-4-(2-oxo-1,7- diazaspiro[3.5]nonan-7-yl)-N-(6- (pyridin-4-yl)thiazolo[4,5-b]pyridin-2- yl)nicotinamide | 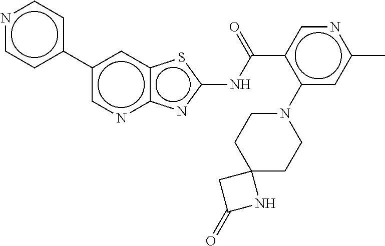 |
| 191 | 4-(2-acetyl-2,7-diazaspiro[3.5]nonan- 7-yl)-6-methyl-N-(6-(pyridin-4- yl)thiazolo[4,5-b]pyridin-2- yl)nicotinamide | 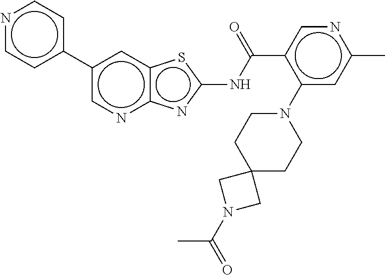 |
| 192 | 6-methyl-4-(2-oxo-1-oxa-3,8- diazaspiro[4.5]decan-8-yl)-N-(6- (pyridin-4-yl)thiazolo[4,5-b]pyridin-2- yl)nicotinamide | 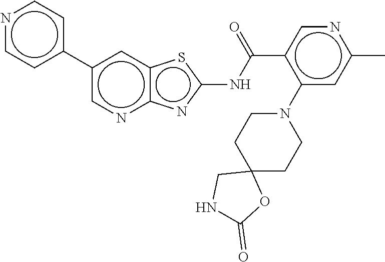 |
| 193 | 6-methyl-4-(3-oxo-2,8- diazaspiro[4.5]decan-8-yl)-N-(6- (pyridin-4-yl)thiazolo[4,5-b]pyridin-2- yl)nicotinamide | 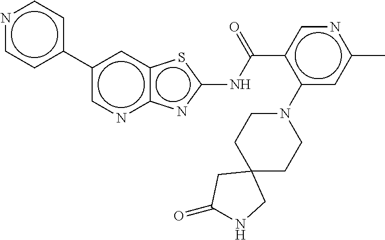 |
| 194 | 4-(2-methoxyphenyl)-6-methyl-N-(7- methylbenzo[d]thiazol-2- yl)nicotinamide | 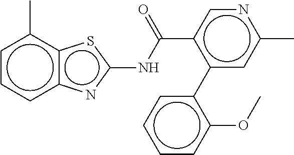 |
| 195 | N-(7-bromobenzo[d]thiazol-2-yl)-4- (2-methoxyphenyl)-6- methylnicotinamide | 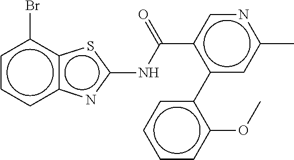 |
| 196 | 4-(2-methoxyphenyl)-6-methyl-N-(7- phenylbenzo[d]thiazol-2- yl)nicotinamide | 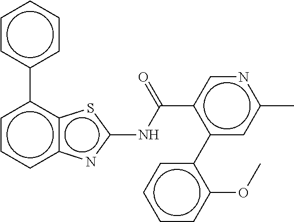 |
| 197 | 4-(2-methoxyphenyl)-6-methyl-N-(4- phenylbenzo[d]thiazol-2- yl)nicotinamide | 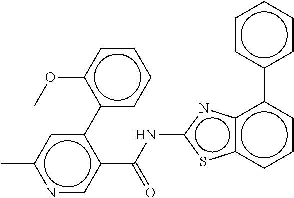 |
| 198 | 4-(2-methoxyphenyl)-6-methyl-N- (pyrazolo[1,5-a]pyridin-2- yl)nicotinamide | 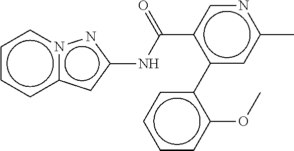 |
| 199 | 4-(7-acetyl-2,7-diazaspiro[3.5]nonan- 2-yl)-6-methyl-N-(6-(pyridin-4- yl)thiazolo[4,5-b]pyridin-2- yl)nicotinamide | 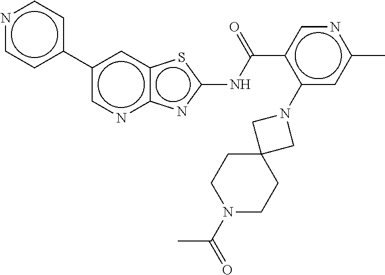 |
| 200 | (S or R)-6-methyl-4-(1-oxo-2,6- diazaspiro[3.5]nonan-6-yl)-N-(6- (pyridin-4-yl)thiazolo[4,5-b]pyrazin- 2-yl)nicotinamide | 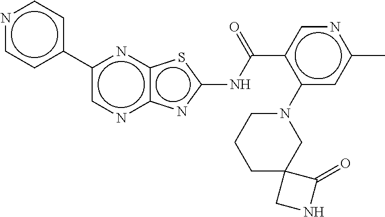 |
| 201 | (Racemic)-6-methyl-4-(8-oxo-2,7- diazaspiro[4.4]nonan-2-yl)-N-(6- (pyridin-4-yl)thiazolo[4,5-b]pyridin-2- yl)nicotinamide | 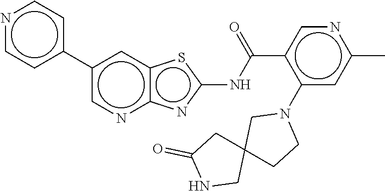 |
| 202 | 4-(6-acetyl-2,6-diazaspiro[3.3]heptan- 2-yl)-6-methyl-N-(6-(pyridin-4- yl)thiazolo[4,5-b]pyridin-2- yl)nicotinamide | 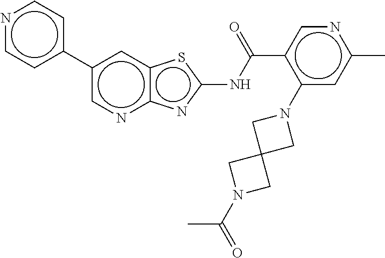 |
| 203 | 4-(2-acetyl-2,8-diazaspiro[4.5]decan- 8-yl)-6-methyl-N-(6-(pyridin-4- yl)thiazolo[4,5-b]pyridin-2- yl)nicotinamide | 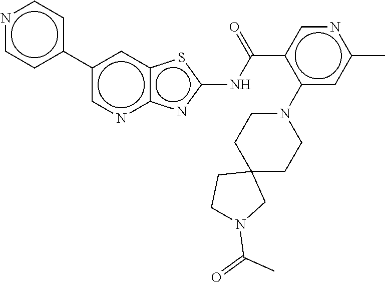 |

TABLE 2

| Compound No. | Name | Structure |
|---|---|---|
| 204 (502) | 2′-chloro-N-(6-(4- (difluoromethoxy)piperidin-1- yl)thiazolo[4,5-b]pyrazin-2-yl)-5′- methoxy-6-methyl-[4,4′-bipyridine]-3- carboxamide | 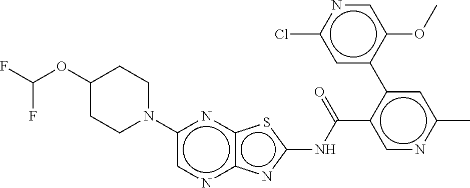 |
| 205 (504) | 2′-chloro-N-(6-(4-(difluoromethyl)-4- hydroxypiperidin-1-yl)thiazolo[4,5- b]pyrazin-2-yl)-5′-methoxy-6-methyl- [4,4′-bipyridine]-3-carboxamide | 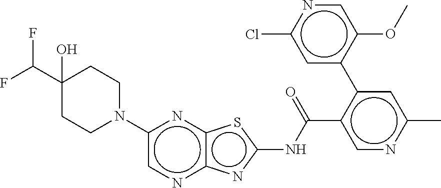 |
| 206 (506) | 2′-chloro-N-(6-(4- (hydroxymethyl)piperidin-1- yl)thiazolo[4,5-b]pyrazin-2-yl)-5′- methoxy-6-methyl-[4,4′-bipyridine]-3- carboxamide | 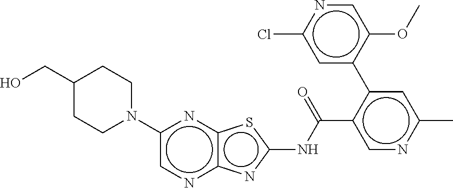 |
| 207 (508) | (Cis and Trans)-2′-chloro-N-(6-(4- hydroxycyclohexyl)thiazolo[4,5- b]pyrazin-2-yl)-5′-methoxy-6-methyl- [4,4′-bipyridine]-3-carboxamide | 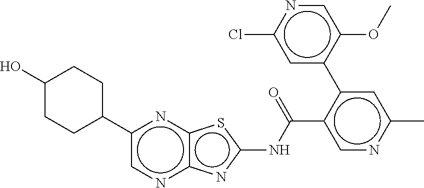 |
| 208 | 2′-chloro-N-(6-((1(R or S),3(S and R) OR 1(R and S),3(S or R))-3- (hydroxymethyl)cyclopentyl)thiazolo[4,5- b]pyrazin-2-yl)-5′-methoxy-6- methyl-[4,4′-bipyridine]-3- carboxamide | 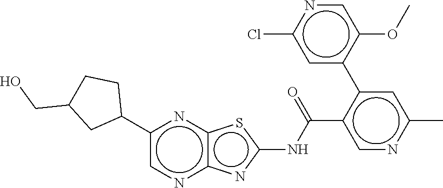 |
| 209 | 2′-chloro-N-(6-((1(R and S),3(S or R) OR 1(R or S),3(S and R))-3- (hydroxymethyl)cyclopentyl)thiazolo[4,5- b]pyrazin-2-yl)-5′-methoxy-6- methyl-[4,4′-bipyridine]-3- carboxamide | 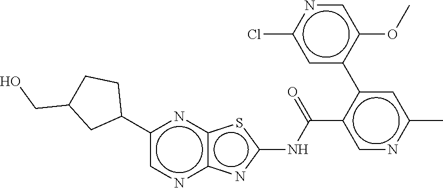 |
| 210 (505) | 2′-chloro-N-(6-(3- (hydroxymethyl)azetidin-1- yl)thiazolo[4,5-b]pyrazin-2-yl)-5′- methoxy-6-methyl-[4,4′-bipyridine]-3- carboxamide | 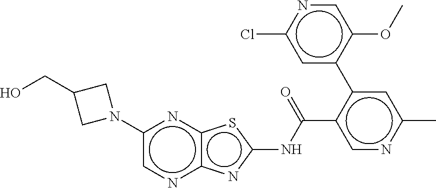 |
| 211 (509) | 2′-chloro-N-(6-(4-hydroxy-4- methylpiperidin-1-yl)thiazolo[4,5- b]pyrazin-2-yl)-5′-methoxy-6-methyl- [4,4′-bipyridine]-3-carboxamide | 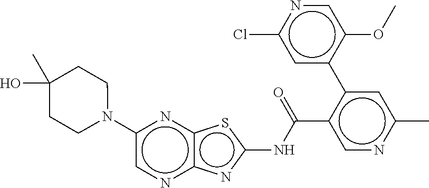 |
| 212 (512) | 2′-chloro-5′-methoxy-6-methyl-N-(6- (tetrahydro-2H-pyran-4- yl)thiazolo[4,5-b]pyrazin-2-yl)-[4,4′- bipyridine]-3-carboxamide | 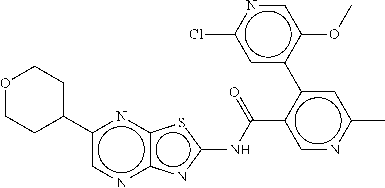 |
| 213 (514) | 2′-chloro-5′-methoxy-N-(6-(4- methoxypiperidin-1-yl)thiazolo[4,5- b]pyrazin-2-yl)-6-methyl-[4,4′- bipyridine]-3-carboxamide | 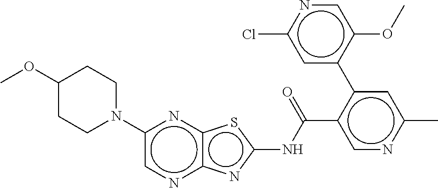 |
| 214 (510) | (Racemic)-2′-chloro-5′-methoxy-6- methyl-N-(6-(tetrahydrofuran-3- yl)thiazolo[4,5-b]pyrazin-2-yl)-[4,4′- bipyridine]-3-carboxamide | 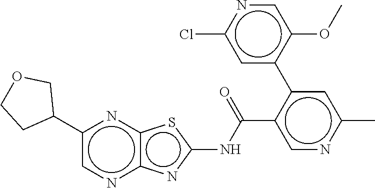 |
| 215 (503) | 2′-chloro-N-(6-(3-hydroxyazetidin-1- yl)thiazolo[4,5-b]pyrazin-2-yl)-5′- methoxy-6-methyl-[4,4′-bipyridine]-3- carboxamide | 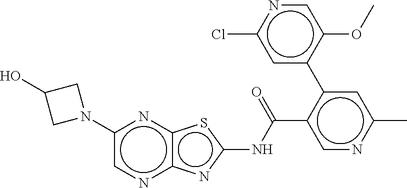 |
| 216 (507) | 4-(6-methoxy-4-methyl-3,4-dihydro- 2H-pyrido[3,2-b][1,4]oxazin-7-yl)-6- methyl-N-(6-(pyridin-4- yl)thiazolo[4,5-b]pyrazin-2- yl)nicotinamide | 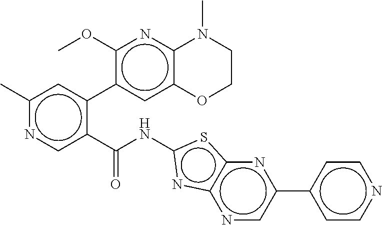 |
| 217 (500) | (S)-2′-chloro-N-(6-(3- (hydroxymethyl)pyrrolidin-1- yl)thiazolo[4,5-b]pyrazin-2-yl)-5′- methoxy-6-methyl-[4,4'-bipyridine]-3- carboxamide | 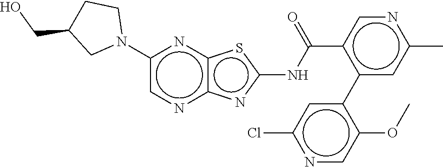 |
| 218 (501) | (R)-2′-chloro-N-(6-(3- (hydroxymethyl)pyrrolidin-1- yl)thiazolo[4,5-b]pyrazin-2-yl)-5′- methoxy-6-methyl-[4,4′-bipyridine]-3- carboxamide | 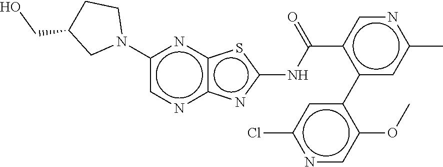 |
| 219 (513) | 6-methyl-4-(2-methyl-2H- pyrazolo[4,3-c]pyridin-3-yl)-N-(6- (pyridin-4-yl)thiazolo[4,5-b]pyrazin-2- yl)nicotinamide | 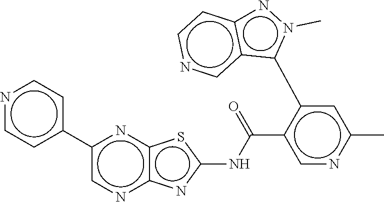 |
| 220 (517) | 6-methyl-4-(1-methyl-1H- pyrazolo[4,3-c]pyridin-3-yl)-N-(6- (pyridin-4-yl)thiazolo[4,5-b]pyrazin-2- yl)nicotinamide | 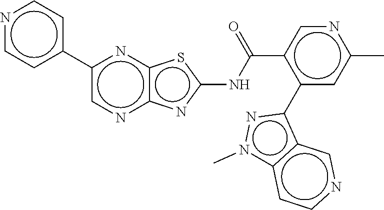 |
| 221 (491) | 2′-chloro-5′-methoxy-6-methyl-N-(6- (piperidin-1-yl)thiazolo[4,5-b]pyrazin- 2-yl)-[4,4′-bipyridine]-3-carboxamide | 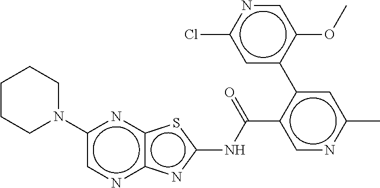 |
| 222 (492) | (Cis and Trans)-2′-chloro-N-(6-(3- (hydroxymethyl)cyclobutyl)thiazolo[4,5- b]pyrazin-2-yl)-5′-methoxy-6- methyl-[4,4′-bipyridine]-3- carboxamide | 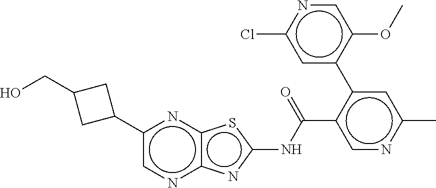 |
| 223 | (Cis or Trans)-2′-chloro-N-(6-(4- (hydroxymethyl)cyclohexyl)thiazolo[4,5- b]pyrazin-2-yl)-5′-methoxy-6- methyl-[4,4′-bipyridine]-3- carboxamide | 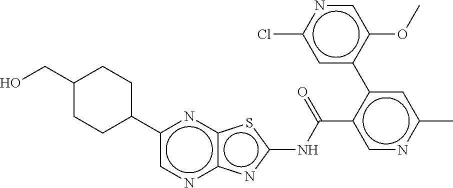 |
| 224 | (Trans or Cis)-2′-chloro-N-(6-(4- (hydroxymethyl)cyclohexyl)thiazolo[4,5- b]pyrazin-2-y1)-5′-methoxy-6- methyl-[4,4′-bipyridine]-3- carboxamide | 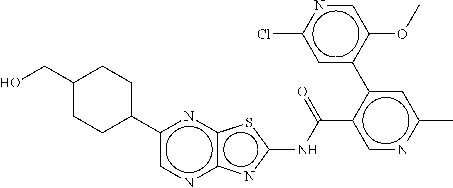 |
| 225 (480) | 2′-chloro-N-(6-(4-(2-hydroxypropan-2- yl)piperidin-1-yl)thiazolo[4,5- b]pyrazin-2-yl)-5′-methoxy-6-methyl- [4,4′-bipyridine]-3-carboxamide | 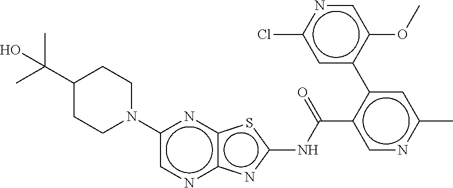 |
| 226 (481) | (S)-2′-chloro-5′-methoxy-6-methyl-N- (6-(3-methylpiperidin-1- yl)thiazolo[4,5-b]pyrazin-2-yl)-[4,4′- bipyridine]-3-carboxamide | 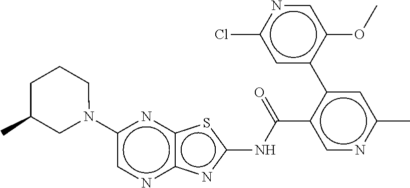 |
| 227 (482) | N-(6-(1-oxa-7-azaspiro[3.5]nonan-7- yl)thiazolo[4,5-b]pyrazin-2-yl)-2′- chloro-5′-methoxy-6-methyl-[4,4′- bipyridine]-3-carboxamide | 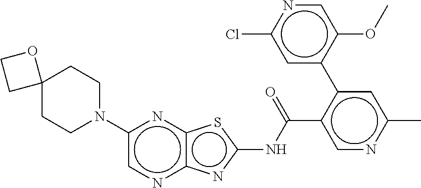 |
| 228 (483) | 2′-chloro-5′-methoxy-6-methyl-N-(6- morpholinothiazolo[4,5-b]pyrazin-2- yl)-[4,4′-bipyridine]-3-carboxamide | 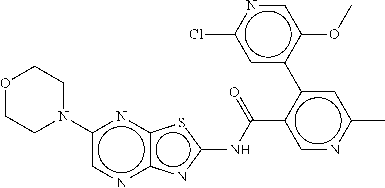 |
| 229 (484) | 2′-chloro-5′-methoxy-6-methyl-N-(6- (pyrrolidin-1-yl)thiazolo[4,5- b]pyrazin-2-yl)-[4,4′-bipyridine]-3- carboxamide | 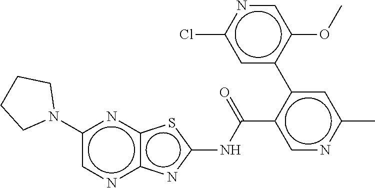 |
| 230 (485) | (R)-2′-chloro-5′-methoxy-6-methyl-N- (6-(3-methylpiperidin-1- yl)thiazolo[4,5-b]pyrazin-2-yl)-[4,4′- bipyridine]-3-carboxamide | 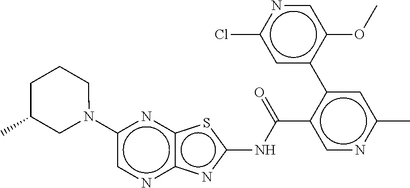 |
| 231 (486) | 2′-chloro-N-(6-(4- (difluoromethyl)piperidin-1- yl)thiazolo[4,5-b]pyrazin-2-yl)-5′- methoxy-6-methyl-[4,4′-bipyridine]-3- carboxamide | 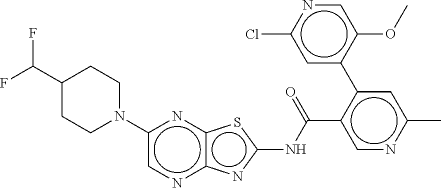 |
| 232 (487) | N-(6-(azetidin-1-yl)thiazolo[4,5- b]pyrazin-2-yl)-2′-chloro-5′-methoxy- 6-methyl-[4,4′-bipyridine]-3- carboxamide | 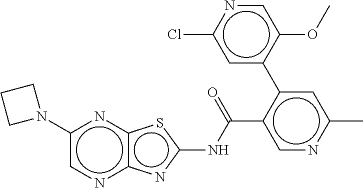 |
| 233 (488) | (R)-2′-chloro-5′-methoxy-6-methyl-N- (6-(2-methylmorpholino)thiazolo[4,5- b]pyrazin-2-yl)-[4,4′-bipyridine]-3- carboxamide | 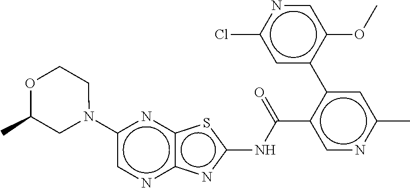 |
| 234 (489) | 2′-chloro-5′-methoxy-6-methyl-N-(6- (4-(trifluoromethoxy)piperidin-1- yl)thiazolo[4,5-b]pyrazin-2-yl)-[4,4′- bipyridine]-3-carboxamide | 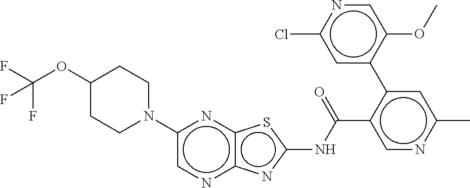 |
| 235 (490) | (S)-2′-chloro-5′-methoxy-6-methyl-N- (6-(2-methylmorpholino)thiazolo[4,5- b]pyrazin-2-yl)-[4,4′-bipyridine]-3- carboxamide | 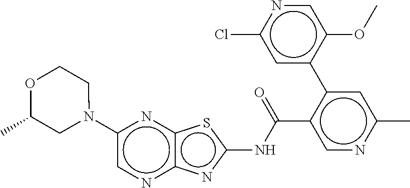 |
| 236 | 2′-chloro-5′-methoxy-6-methyl-N-(6- (2-methylmorpholino)thiazolo[4,5- b]pyrazin-2-yl)-[4,4′-bipyridine]-3- carboxamide | 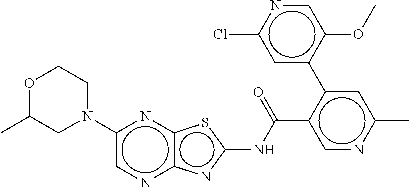 |
| 237 | 2′-chloro-5′-methoxy-6-methyl-N-(6- (3-methylpiperidin-1-yl)thiazolo[4,5- b]pyrazin-2-yl)-[4,4′-bipyridine]-3- carboxamide | 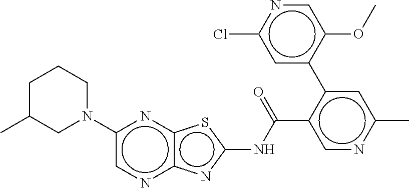 |
| 238 | 2′-chloro-N-(6-(4-hydroxy-3- methylpiperidin-1-yl)thiazolo[4,5- b]pyrazin-2-yl)-5′-methoxy-6-methyl- [4,4′-bipyridine]-3-carboxamide | 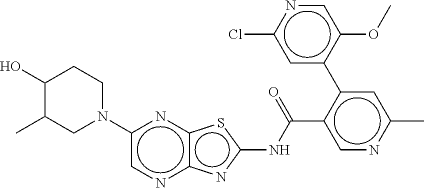 |
| 239 (471) | 2′-chloro-5′-methoxy-6-methyl-N-(6- (4-(2,2,2-trifluoroethyl)piperazin-1- yl)thiazolo[4,5-b]pyrazin-2-yl)-[4,4′- bipyridine]-3-carboxamide | 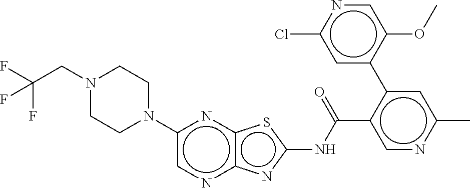 |
| 240 (474) | 2′-chloro-5′-methoxy-6-methyl-N-(6- (4-methylpiperidin-1-yl)thiazolo[4,5- b]pyrazin-2-yl)-[4,4′-bipyridine]-3- carboxamide | 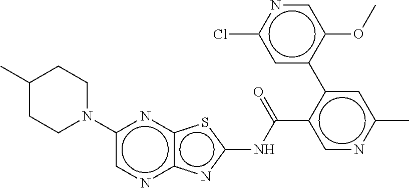 |
| 241 (472) | 2′-chloro-5′-methoxy-6-methyl-N-(6- (4-(trifluoromethyl)piperidin-1- yl)thiazolo[4,5-b]pyrazin-2-yl)-[4,4′- bipyridine]-3-carboxamide | 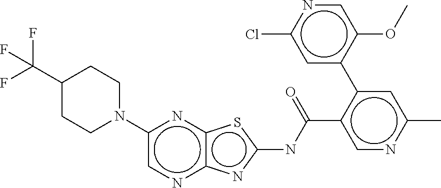 |
| 242 (466) | 2′-chloro-N-(6-(4- cyclopropylpiperidin-1- yl)thiazolo[4,5-b]pyrazin-2-yl)-5′- methoxy-6-methyl-[4,4′-bipyridine]-3- carboxamide | 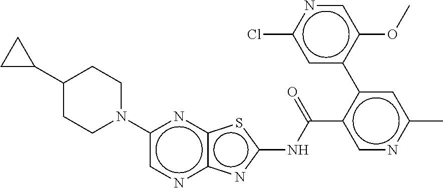 |
| 243 | 2′-chloro-N-(6-(3-fluoro-4- hydroxypiperidin-1-yl)thiazolo[4,5- b]pyrazin-2-yl)-5′-methoxy-6-methyl- [4,4′-bipyridine]-3-carboxamide | 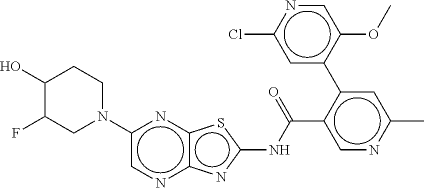 |
| 244 (465) | 2′-chloro-N-(6-(3-fluoro-4-hydroxy-4- methylpiperidin-1-yl)thiazolo[4,5- b]pyrazin-2-yl)-5′-methoxy-6-methyl- [4,4′-bipyridine]-3-carboxamide | 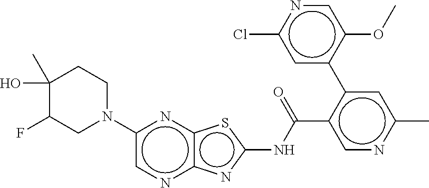 |
| 245 | 2′-chloro-N-(6-(3,3-difluoro-4- hydroxypiperidin-1-yl)thiazolo[4,5- b]pyrazin-2-yl)-5′-methoxy-6-methyl- [4,4′-bipyridine]-3-carboxamide | 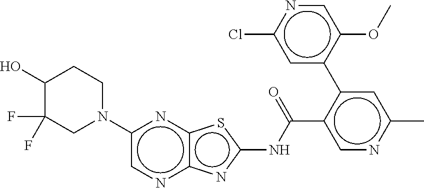 |
| 246 (475) | 2′-chloro-N-(6-(4-cyanopiperidin-1- yl)thiazolo[4,5-b]pyrazin-2-yl)-5′- methoxy-6-methyl-[4,4′-bipyridine]-3- carboxamide | 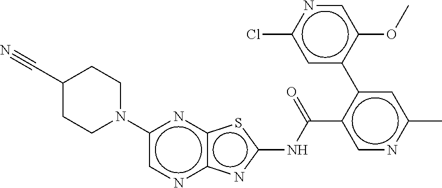 |
| 247 (467) | 2′-chloro-N-(6-(4- (cyanomethyl)piperidin-1- yl)thiazolo[4,5-b]pyrazin-2-yl)-5′- methoxy-6-methyl-[4,4′-bipyridine]-3- carboxamide | 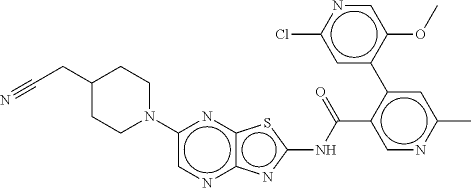 |
| 248 (468) | 2′-chloro-N-(6-(4-(2-cyanopropan-2- yl)piperidin-1-yl)thiazolo[4,5- b]pyrazin-2-yl)-5′-methoxy-6-methyl- [4,4′-bipyridine]-3-carboxamide | 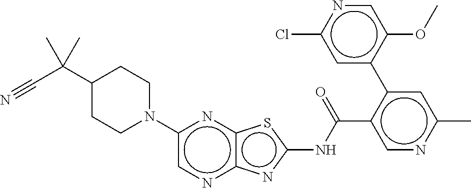 |
| 249 (477) | 2′-chloro-5′-methoxy-6-methyl-N-(6- (4-(methylamino)piperidin-1- yl)thiazolo[4,5-b]pyrazin-2-yl)-[4,4′- bipyridine]-3-carboxamide | 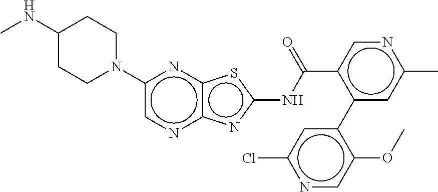 |
| 250 (469) | 2′-chloro-N-(6-(4- (dimethylamino)piperidin-1- yl)thiazolo[4,5-b]pyrazin-2-yl)-5′- methoxy-6-methyl-[4,4′-bipyridine]-3- carboxamide | 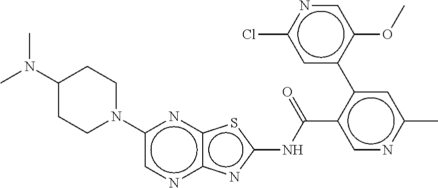 |
| 251 (462) | 2′-chloro-N-(6-(4- cyclopropoxypiperidin-1- yl)thiazolo[4,5-b]pyrazin-2-yl)-5′- methoxy-6-methyl-[4,4′-bipyridine]-3- carboxamide | 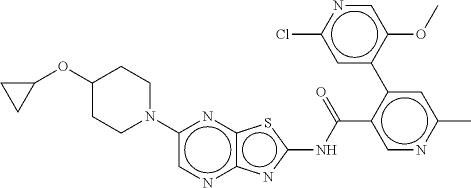 |
| 252 (464) | 2′-chloro-5′-methoxy-6-methyl-N-(6- (4-(oxetan-3-yl)piperidin-1- yl)thiazolo[4,5-b]pyrazin-2-yl)-[4,4′- bipyridine]-3-carboxamide | 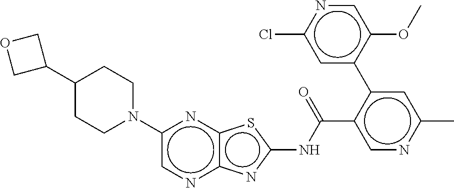 |
| 253 (455) | 2′-chloro-N-(6-(3,3-difluoro-4- (methylamino)piperidin-1- yl)thiazolo[4,5-b]pyrazin-2-yl)-5′- methoxy-6-methyl-[4,4′-bipyridine]-3- carboxamide | 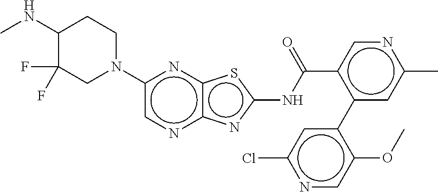 |
| 254 | 2′-chloro-N-(6-(3,3-difluoro-4- methoxypiperidin-1-yl)thiazolo[4,5- b]pyrazin-2-yl)-5′-methoxy-6-methyl- [4,4′-bipyridine]-3-carboxamide | 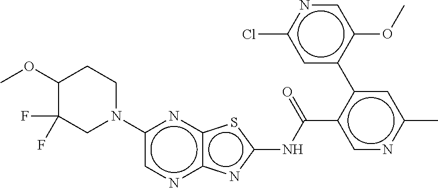 |
| 255 | 2′-chloro-N-(6-(3-fluoro-4- methoxypiperidin-1-yl)thiazolo[4,5- b]pyrazin-2-yl)-5′-methoxy-6-methyl- [4,4′-bipyridine]-3-carboxamide | 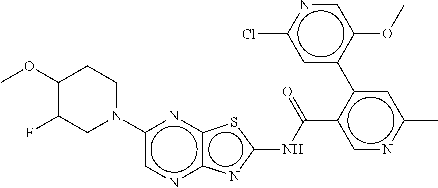 |
| 256 (459) | N-(6-(1-oxa-8-azaspiro[4.5]decan-8- yl)thiazolo[4,5-b]pyrazin-2-yl)-2′- chloro-5′-methoxy-6-methyl-[4,4′- bipyridine]-3-carboxamide | 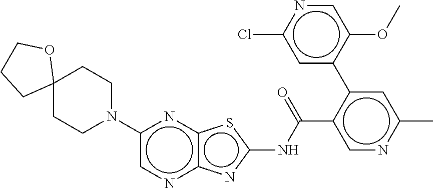 |
| 257 | 2′-chloro-N-(6-(4-(2-hydroxypropan-2- yl)cyclohexyl)thiazolo[4,5-b]pyrazin- 2-yl)-5′-methoxy-6-methyl-[4,4′- bipyridine]-3-carboxamide | 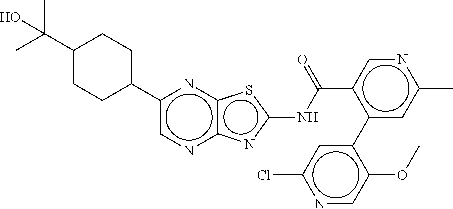 |
| 258 | 2′-chloro-5′-methoxy-N-(6-(4- methoxycyclohexyl)thiazolo[4,5- b]pyrazin-2-yl)-6-methyl-[4,4′- bipyridine]-3-carboxamide | 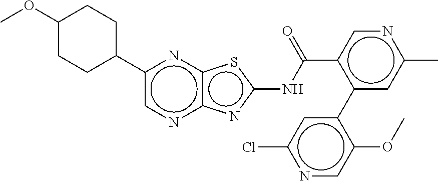 |
| 259 | 2′-chloro-N-(6-(4-hydroxy-4- methylcyclohexyl)thiazolo[4,5- b]pyrazin-2-yl)-5′-methoxy-6-methyl- [4,4′-bipyridine]-3-carboxamide | 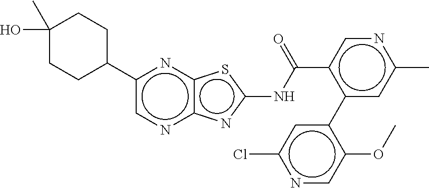 |
| 260 | 2′-chloro-5'-methoxy-6-methyl-N-(6- ((2R)-2-methyltetrahydro-2H-pyran-4- yl)thiazolo[4,5-b]pyrazin-2-yl)-[4,4′- bipyridine]-3-carboxamide | 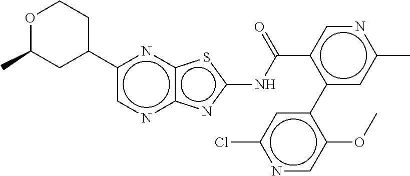 |
| 261 | 2′-chloro-5′-methoxy-N-(6-(4- methoxy-4- methylcyclohexyl)thiazolo[4,5- b]pyrazin-2-yl)-6-methyl-[4,4′- bipyridine]-3-carboxamide | 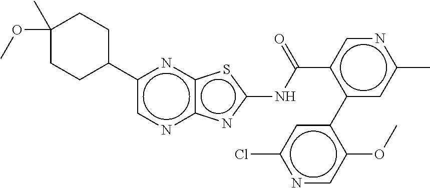 |
| 262 | 2′-chloro-5′-methoxy-6-methyl-N-(6- ((2S)-2-methyltetrahydro-2H-pyran-4- yl)thiazolo[4,5-b]pyrazin-2-yl)-[4,4′- bipyridine]-3-carboxamide | 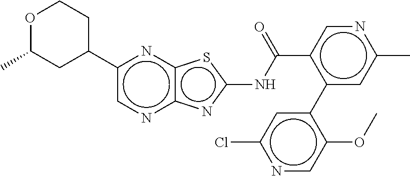 |
| 263 | 2′-chloro-N-(6-(4- cyanocyclohexyl)thiazolo[4,5- b]pyrazin-2-yl)-5′-methoxy-6-methyl- [4,4′-bipyridine]-3-carboxamide | 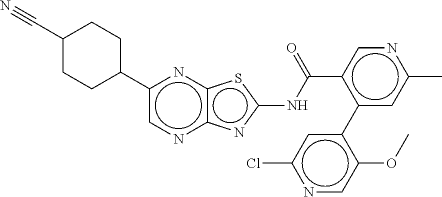 |
| 264 (448) | 2′-chloro-N-(6-(2- (difluoromethyl)pyridin-4- yl)thiazolo[4,5-b]pyrazin-2-yl)-5′- methoxy-6-methyl-[4,4′-bipyridine]-3- carboxamide | 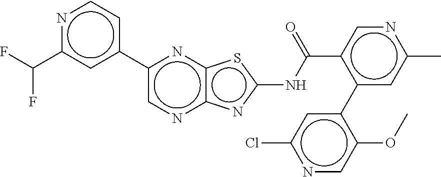 |
| 265 (446) | 2′-chloro-N-(6-(6- (difluoromethyl)pyridin-2- yl)thiazolo[4,5-b]pyrazin-2-yl)-5′- methoxy-6-methyl-[4,4′-bipyridine]-3- carboxamide | 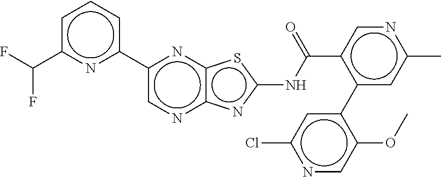 |

TABLE 3

| Compound No. | Name | Structure |
|---|---|---|
| 266 | (Cis and Trans)-2′-chloro-N-(6-(4- hydroxycyclohexyl)imidazo[2,1- b][1,3,4]thiadiazol-2-yl)-5′-methoxy- 6-methyl-[4,4′-bipyridine]-3- carboxamide | 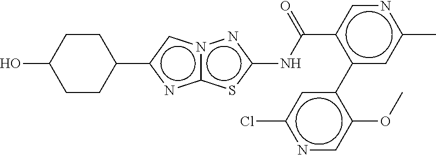 |
| 267 | (S)-2′-chloro-N-(6-(3- hydroxypyrrolidine-1- carbonyl)imidazo[2,1- b][1,3,4]thiadiazol-2-yl)-5′-methoxy- 6-methyl-[4,4′-bipyridine]-3- carboxamide | 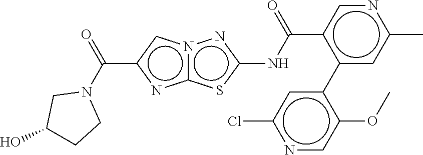 |
| 268 | (R)-2′-chloro-N-(6-(3- hydroxypyrrolidine-1- carbonyl)imidazo[2,1- b][1,3,4]thiadiazol-2-yl)-5′-methoxy- 6-methyl-[4,4′-bipyridine]-3- carboxamide | 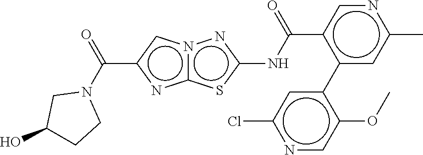 |
| 269 | 2′-chloro-N-(6-((1(R or S),3(R and S) OR (1(R and S),3(R or S)-3- hydroxycyclopentyl)imidazo[2,1- b][1,3,4]thiadiazol-2-yl)-5′-methoxy- 6-methyl-[4,4′-bipyridine]-3- carboxamide | 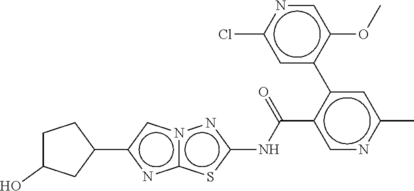 |
| 270 | (Racemic)-2′-chloro-N-(6-(3- (hydroxymethyl)cyclopentyl)imidazo[2,1- b][1,3,4]thiadiazol-2-yl)-5′- methoxy-6-methyl-[4,4′-bipyridine]-3- carboxamide | 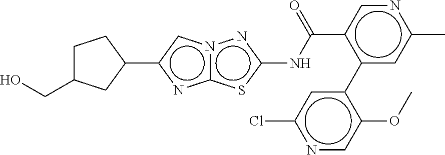 |
| 271 | (Cis and Trans)-2′-chloro-N-(6-(3- hydroxycyclobutyl)imidazo[2,1- b][1,3,4]thiadiazol-2-yl)-5′-methoxy- 6-methyl-[4,4′-bipyridine]-3- carboxamide | 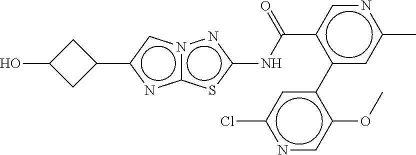 |
| 272 | 2′-chloro-N-(6-(3-hydroxyazetidine-1- carbonyl)imidazo[2,1- b][1,3,4]thiadiazol-2-yl)-5′-methoxy- 6-methyl-[4,4′-bipyridine]-3- carboxamide | 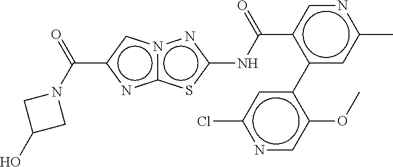 |
| 273 | 2′-chloro-N-(6-((1(R and S),3(R or S) OR (1(R or S),3(R and S)-3- hydroxycyclopentyl)imidazo[2,1- b][1,3,4]thiadiazol-2-yl)-5′-methoxy- 6-methyl-[4,4′-bipyridine]-3- carboxamide | 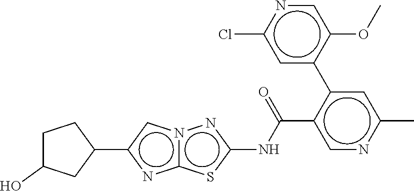 |
| 274 | 2-(2′-chloro-5′-methoxy-6-methyl- [4,4′-bipyridine]-3-carboxamido)-N- ((1S,3R)-3- hydroxycyclohexyl)imidazo[2,1- b][1,3,4]thiadiazole-6-carboxamide | 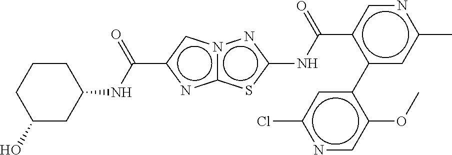 |
| 275 | 2′-chloro-5′-methoxy-6-methyl-N-(6- (pyrrolidine-1-carbonyl)imidazo[2,1- b][1,3,4]thiadiazol-2-yl)-[4,4′- bipyridine]-3-carboxamide | 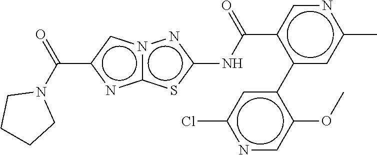 |
| 276 | 2′-chloro-N-(6-(3- (difluoromethoxy)azetidine-1- carbonyl)imidazo[2,1- b][1,3,4]thiadiazol-2-yl)-5′-methoxy- 6-methyl-[4,4′-bipyridine]-3- carboxamide | 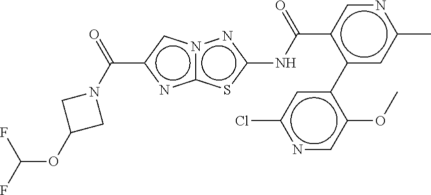 |
| 277 | 2′-chloro-N-(6-(4-hydroxypiperidine- 1-carbonyl)imidazo[2,1- b][1,3,4]thiadiazol-2-yl)-5′-methoxy- 6-methyl-[4,4′-bipyridine]-3- carboxamide | 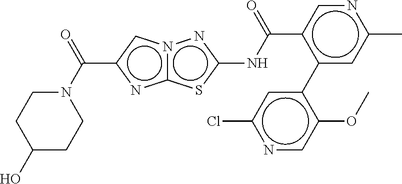 |
| 278 | 2-(2′-chloro-5′-methoxy-6-methyl- [4,4′-bipyridine]-3-carboxamido)-N- ((1S,2R)-2- hydroxycyclohexyl)imidazo[2,1- b][1,3,4]thiadiazole-6-carboxamide | 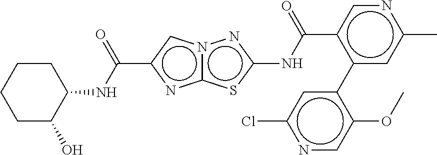 |
| 279 | (Cis or Trans)-2′-chloro-N-(6-(3- (difluoromethyl)cyclobutyl)imidazo[2,1- b][1,3,4]thiadiazol-2-yl)-5′- methoxy-6-methyl-[4,4′-bipyridine]-3- carboxamide | 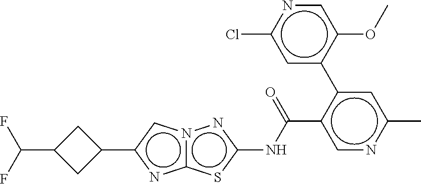 |
| 280 | 2-(2′-chloro-5′-methoxy-6-methyl- [4,4′-bipyridine]-3-carboxamido)-N- ((1S,3S)-3- hydroxycyclohexyl)imidazo[2,1- b][1,3,4]thiadiazole-6-carboxamide | 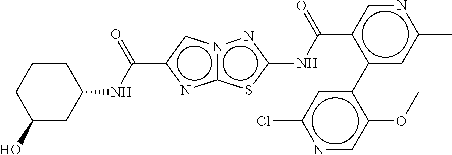 |
| 281 | 2-(2′-chloro-5′-methoxy-6-methyl- [4,4′-bipyridine]-3-carboxamido)-N- ((1S,2S)-2- hydroxycyclohexyl)imidazo[2,1- b][1,3,4]thiadiazole-6-carboxamide | 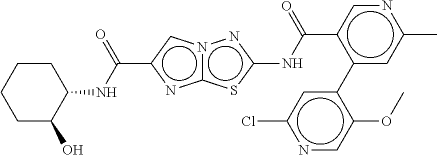 |
| 282 | 2-(2′-chloro-5′-methoxy-6-methyl- [4,4′-bipyridine]-3-carboxamido)-N- ((1s,3s)-3-hydroxy-3- (trifluoromethyl)cyclobutyl)imidazo[2,1- b][1,3,4]thiadiazole-6-carboxamide | 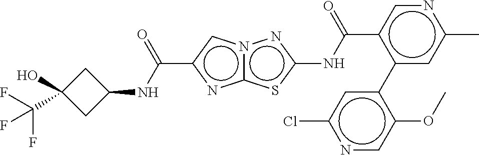 |
| 283 | (Trans or Cis)-2′-chloro-N-(6-(3- (difluoromethyl)cyclobutyl)imidazo[2,1- b][1,3,4]thiadiazol-2-yl)-5′- methoxy-6-methyl-[4,4′-bipyridine]-3- carboxamide | 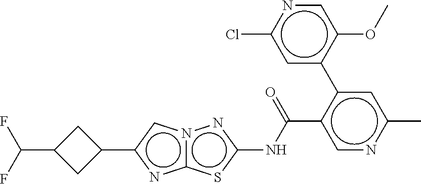 |
| 284 | (Racemic)-2′-chloro-N-(6-(2,2- difluorocyclopropyl)imidazo[2,1- b][1,3,4]thiadiazol-2-yl)-5′-methoxy- 6-methyl-[4,4′-bipyridine]-3- carboxamide | 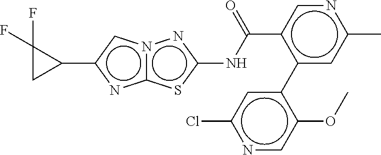 |
| 285 | 2-(2′-chloro-5′-methoxy-6-methyl- [4,4′-bipyridine]-3-carboxamido)-N- ((1r,3r)-3-hydroxy-3- (trifluoromethyl)cyclobutyl)imidazo[2,1- b][1,3,4]thiadiazole-6-carboxamide | 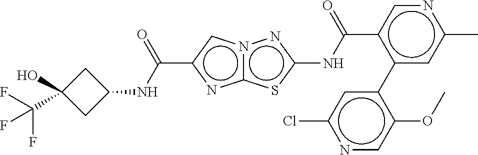 |
| 286 | (Racemic)-2′-chloro-5′-methoxy-N-(6- (2-methoxycyclopropyl)imidazo[2,1- b][1,3,4]thiadiazol-2-yl)-6-methyl- [4,4′-bipyridine]-3-carboxamide | 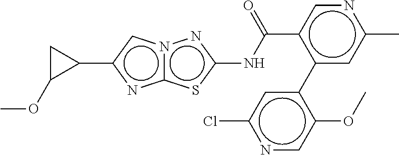 |
| 287 | 2-(2′-chloro-5′-methoxy-6-methyl- [4,4′-bipyridine]-3-carboxamido)-N- cyclopropylimidazo[2,1- b][1,3,4]thiadiazole-6-carboxamide | 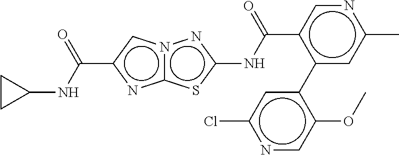 |
| 288 | 2-(2′-chloro-5′-methoxy-6-methyl- [4,4′-bipyridine]-3-carboxamido)-N- cyclobutylimidazo[2,1- b][1,3,4]thiadiazole-6-carboxamide | 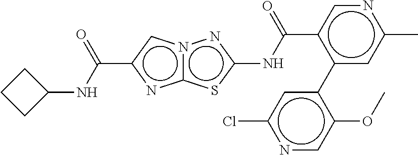 |
| 289 | (R)-2-(2′-chloro-5′-methoxy-6-methyl- [4,4′-bipyridine]-3-carboxamido)-N- (tetrahydrofuran-3-yl)imidazo[2,1- b][1,3,4]thiadiazole-6-carboxamide | 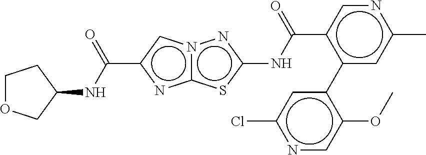 |
| 290 | 2-(2′-chloro-5′-methoxy-6-methyl- [4,4′-bipyridine]-3-carboxamido)-N- (oxetan-3-yl)imidazo[2,1- b][1,3,4]thiadiazole-6-carboxamide | 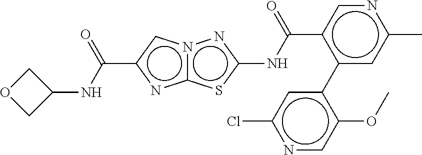 |
| 291 | (S)-2-(2′-chloro-5′-methoxy-6-methyl- [4,4′-bipyridine]-3-carboxamido)-N- (tetrahydrofuran-3-yl)imidazo[2,1- b][1,3,4]thiadiazole-6-carboxamide | 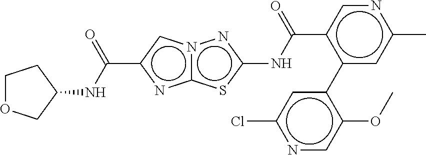 |
| 292 | 2-(2′-chloro-5′-methoxy-6-methyl- [4,4′-bipyridine]-3-carboxamido)-N- ((1s,4s)-4- hydroxycyclohexyl)imidazo[2,1- b][1,3,4]thiadiazole-6-carboxamide | 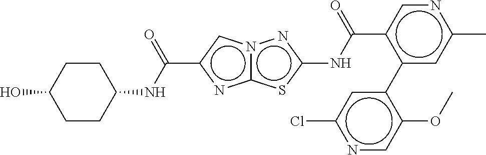 |
| 293 | 2-(2′-chloro-5′-methoxy-6-methyl- [4,4′-bipyridine]-3-carboxamido)-N- ((1R,2S)-2- hydroxycyclohexyl)imidazo[2,1- b][1,3,4]thiadiazole-6-carboxamide | 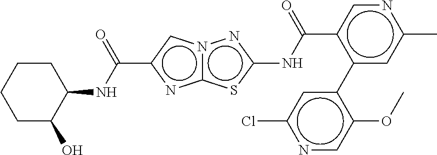 |
| 294 | 2-(2′-chloro-5′-methoxy-6-methyl- [4,4′-bipyridine]-3-carboxamido)-N- ((1r,4r)-4- hydroxycyclohexyl)imidazo[2,1- b][1,3,4]thiadiazole-6-carboxamide | 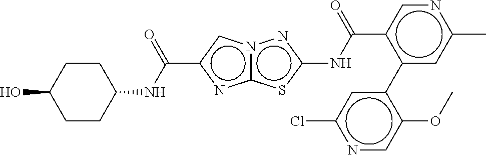 |
| 295 | 2-(2′-chloro-5′-methoxy-6-methyl- [4,4′-bipyridine]-3-carboxamido)-N- ((1R,3S)-3- hydroxycyclopentyl)imidazo[2,1- b][1,3,4]thiadiazole-6-carboxamide | 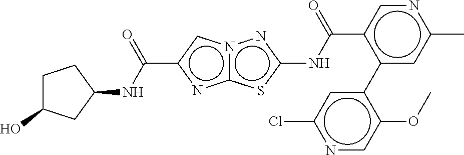 |
| 296 | (Cis and Trans)-2′-chloro-5′-methoxy- N-(6-(3- methoxycyclobutyl)imidazo[2,1- b][1,3,4]thiadiazol-2-yl)-6-methyl- [4,4′-bipyridine]-3-carboxamide | 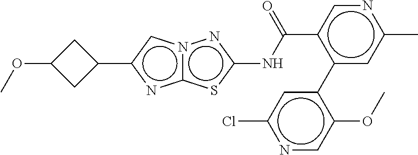 |
| 297 | 2-(2′-chloro-5′-methoxy-6-methyl- [4,4′-bipyridine]-3-carboxamido)-N- ((1R,2R)-2- hydroxycyclohexyl)imidazo[2,1- b][1,3,4]thiadiazole-6-carboxamide | 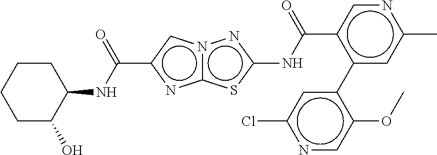 |
| 298 | 2-(2′-chloro-5′-methoxy-6-methyl- [4,4′-bipyridine]-3-carboxamido)-N- ((1S,3S)-3- hydroxycyclopentyl)imidazo[2,1- b][1,3,4]thiadiazole-6-carboxamide | 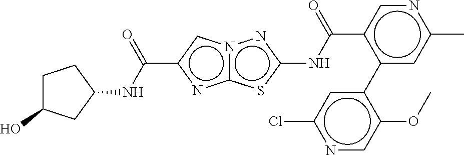 |
| 299 | 2-(2′-chloro-5′-methoxy-6-methyl- [4,4′-bipyridine]-3-carboxamido)-N- ((1R,3S)-3- hydroxycyclohexyl)imidazo[2,1- b][1,3,4]thiadiazole-6-carboxamide | 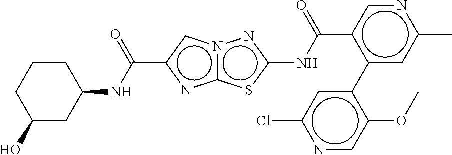 |
| 300 | 2-(2′-chloro-5′-methoxy-6-methyl- [4,4′-bipyridine]-3-carboxamido)-N- ((1r,3r)-3-hydroxy-3- methylcyclobutyl)imidazo[2,1- b][1,3,4]thiadiazole-6-carboxamide | 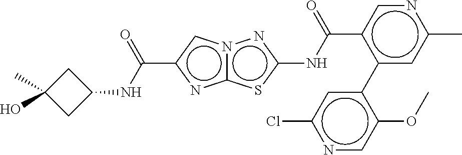 |
| 301 | 2-(2′-chloro-5′-methoxy-6-methyl- [4,4′-bipyridine]-3-carboxamido)-N- ((1R,3R)-3- hydroxycyclohexyl)imidazo[2,1- b][1,3,4]thiadiazole-6-carboxamide | 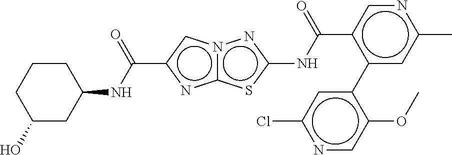 |
| 302 | 2-(2′-chloro-5′-methoxy-6-methyl- [4,4′-bipyridine]-3-carboxamido)-N- (3-(difluoromethyl)-3- hydroxycyclobutyl)imidazo[2,1- b][1,3,4]thiadiazole-6-carboxamide | 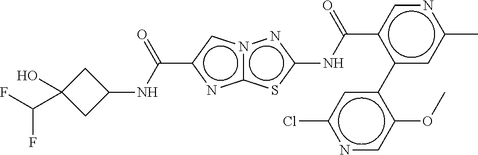 |
| 303 | (Racemic)-2′-chloro-5′-methoxy-6- methyl-N-(6-(2- methylcyclopropyl)imidazo[2,1- b][1,3,4]thiadiazol-2-y1)-[4,4′- bipyridine]-3-carboxamide | 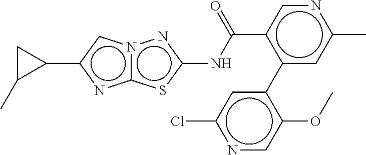 |
| 304 | N3′-(6-cyclopropylimidazo[2,1- b][1,3,4]thiadiazol-2-yl)-5-methoxy- 6′-methyl-[4,4′-bipyridine]-2,3′- dicarboxamide | 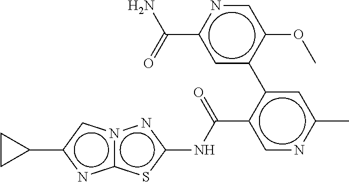 |
| 305 | 2-(2′-chloro-5′-methoxy-6-methyl- [4,4′-bipyridine]-3-carboxamido)-N- ((1R,3R)-3- hydroxycyclopentyl)imidazo[2,1- b][1,3,4]thiadiazole-6-carboxamide | 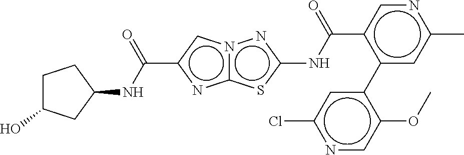 |
| 306 | 2-(2′-chloro-5′-methoxy-6-methyl- [4,4′-bipyridine]-3-carboxamido)-N- ((1S,2R)-2- hydroxycyclopentyl)imidazo[2,1- b][1,3,4]thiadiazole-6-carboxamide | 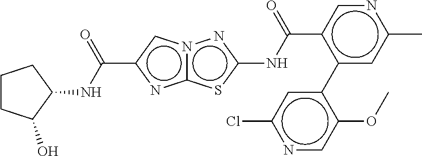 |
| 307 | 2-(2′-chloro-5′-methoxy-6-methyl- [4,4′-bipyridine]-3-carboxamido)-N- ((1R,2S)-2- hydroxycyclopentyl)imidazo[2,1- b][1,3,4]thiadiazole-6-carboxamide | 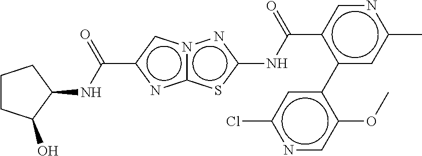 |
| 308 | 2-(2′-chloro-5′-methoxy-6-methyl- [4,4′-bipyridine]-3-carboxamido)-N- ((1s,3s)-3- hydroxycyclobutyl)imidazo[2,1- b][1,3,4]thiadiazole-6-carboxamide | 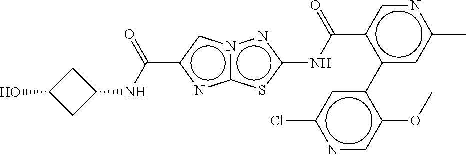 |
| 309 | 2-(2′-chloro-5′-methoxy-6-methyl- [4,4′-bipyridine]-3-carboxamido)-N- ((1S,2S)-2- hydroxycyclopentyl)imidazo[2,1- b][1,3,4]thiadiazole-6-carboxamide | 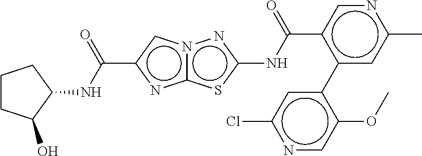 |
| 310 | 2-(2′-chloro-5′-methoxy-6-methyl- [4,4′-bipyridine]-3-carboxamido)-N- ((1R,2R)-2- hydroxycyclopentyl)imidazo[2,1- b][1,3,4]thiadiazole-6-carboxamide | 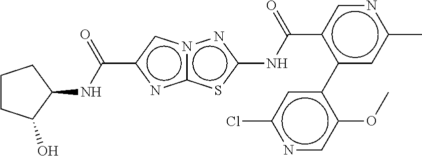 |
| 311 | 2-(2′-chloro-5′-methoxy-6-methyl- [4,4′-bipyridine]-3-carboxamido)-N- ((1r,3r)-3- hydroxycyclobutyl)imidazo[2,1- b][1,3,4]thiadiazole-6-carboxamide | 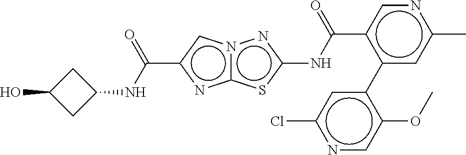 |
| 312 | 2-(2′-chloro-5′-methoxy-6-methyl- [4,4′-bipyridine]-3-carboxamido)-N- ((1S,3R)-3- hydroxycyclopentyl)imidazo[2,1- b][1,3,4]thiadiazole-6-carboxamide | 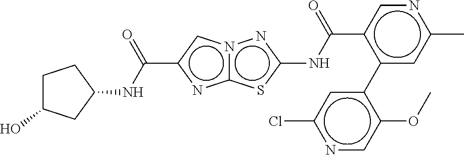 |
| 313 | 2-(2′-chloro-5′-methoxy-6-methyl- [4,4′-bipyridine]-3-carboxamido)-N- ((1s,3s)-3-hydroxy-3- methylcyclobutyl)imidazo[2,1- b][1,3,4]thiadiazole-6-carboxamide | 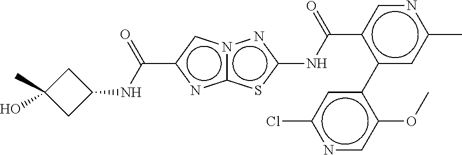 |
| 314 | (Racemic)-2′-chloro-N-(6-(2- (difluoromethyl)cyclopropyl)imidazo[2,1- b][1,3,4]thiadiazol-2-yl)-5′- methoxy-6-methyl-[4,4′-bipyridine]-3- carboxamide | 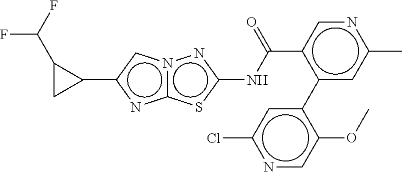 |
| 315 | 2′-chloro-N-(6- cyclohexylimidazo[2,1- b][1,3,4]thiadiazol-2-yl)-5′-methoxy- 6-methyl-[4,4′-bipyridine]-3- carboxamide | 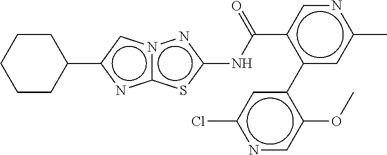 |
| 316 | 2-(4-(5-cyano-2-methoxyphenyl)-6- methylnicotinamido)-N,N- dimethylimidazo[2,1- b][1,3,4]thiadiazole-6-carboxamide | 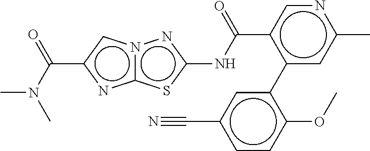 |
| 317 | 2′-chloro-5′-methoxy-6-methyl-N-(6- (1-methylcyclopropyl)imidazo[2,1- b][1,3,4]thiadiazol-2-yl)-[4,4′- bipyridine]-3-carboxamide | 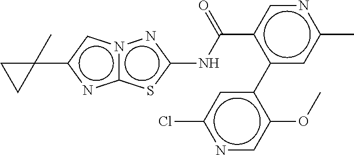 |
| 318 | 2′-chloro-N-(6-cyclobutylimidazo[2,1- b][1,3,4]thiadiazol-2-yl)-5′-methoxy- 6-methyl-[4,4′-bipyridine]-3- carboxamide | 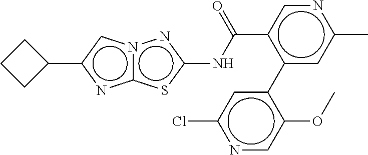 |
| 319 | 2′-chloro-N-(5-chloro-6- cyclopropylimidazo[2,1- b][1,3,4]thiadiazol-2-yl)-6-cyano-5′- methoxy-[4,4′-bipyridine]-3- carboxamide | 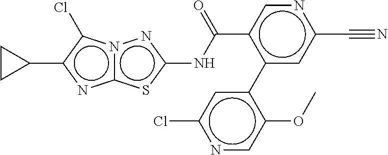 |
| 320 | 2′-chloro-N-(6- cyclopentylimidazo[2,1- b][1,3,4]thiadiazol-2-yl)-5′-methoxy- 6-methyl-[4,4′-bipyridine]-3- carboxamide | 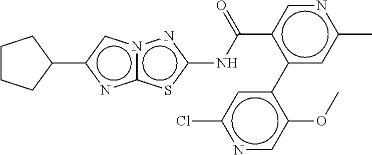 |
| 321 | (Racemic)-2′-chloro-5′-methoxy-6- methyl-N-(6-(spiro[2.2]pentan-1- yl)imidazo[2,1-b][1,3,4]thiadiazol-2- yl)-[4,4′-bipyridine]-3-carboxamide | 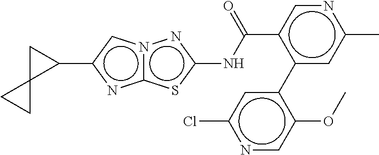 |
| 322 | 4-(2-methoxyphenyl)-6-methyl-N-(6- (pyrrolidine-1-carbonyl)imidazo[2,1- b][1,3,4]thiadiazol-2-yl)nicotinamide | 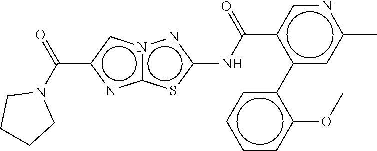 |
| 323 | 2′-chloro-6-cyano-N-(6- cyclopropylimidazo[2,1- b][1,3,4]thiadiazol-2-yl)-5′-methoxy- [4,4′-bipyridine]-3-carboxamide | 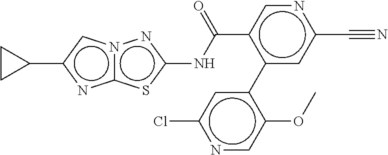 |
| 324 | (R or S)-2′-chloro-5′-methoxy-6- methyl-N-(6-(tetrahydrofuran-3- yl)imidazo[2,1-b][1,3,4]thiadiazol-2- yl)-[4,4′-bipyridine]-3-carboxamide | 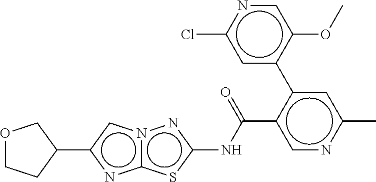 |
| 325 | N-(6-(1- methoxycyclopropyl)imidazo[2,1- b][1,3,4]thiadiazol-2-yl)-4-(2- methoxyphenyl)-6- methylnicotinamide | 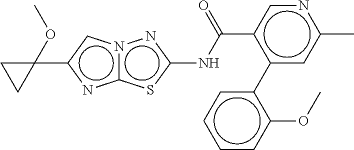 |
| 326 | (S or R)-2′-chloro-5′-methoxy-6- methyl-N-(6-(tetrahydrofuran-3- yl)imidazo[2,1-b][1,3,4]thiadiazol-2- yl)-[4,4′-bipyridine]-3-carboxamide | 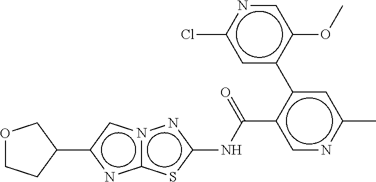 |
| 327 | (R or S)-4-(5-cyano-2- methoxyphenyl)-6-methyl-N-(6- (tetrahydrofuran-3-yl)imidazo[2,1- b][1,3,4]thiadiazol-2-yl)nicotinamide | 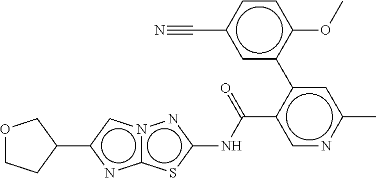 |
| 328 | (S or R)-4-(5-cyano-2- methoxyphenyl)-6-methyl-N-(6- (tetrahydrofuran-3-yl)imidazo[2,1- b][1,3,4]thiadiazol-2-yl)nicotinamide | 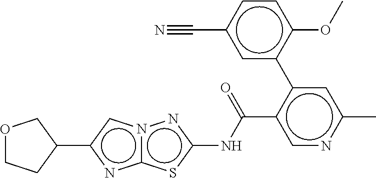 |
| 329 | N-(6-(azetidine-1- carbonyl)imidazo[2,1- b][1,3,4]thiadiazol-2-yl)-4-(2- methoxyphenyl)-6- methylnicotinamide | 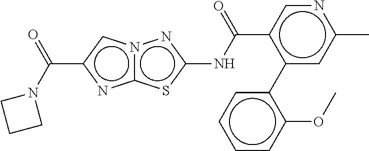 |
| 330 | (S)-2-(4-(2-methoxyphenyl)-6- methylnicotinamido)-N- (tetrahydrofuran-3-yl)imidazo[2,1- b][1,3,4]thiadiazole-6-carboxamide | 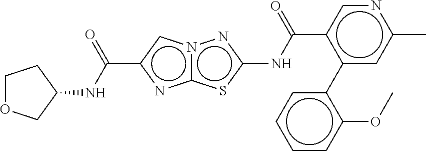 |
| 331 | 4-(2-methoxyphenyl)-6-methyl-N-(6- (morpholine-4-carbonyl)imidazo[2,1- b][1,3,4]thiadiazol-2-yl)nicotinamide | 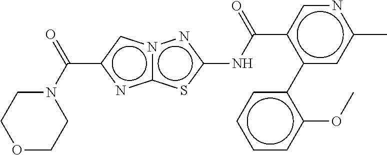 |
| 332 | (R)-2-(4-(2-methoxyphenyl)-6- methylnicotinamido)-N- (tetrahydrofuran-3-yl)imidazo[2,1- b][1,3,4]thiadiazole-6-carboxamide | 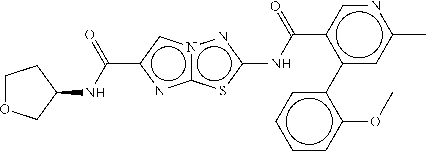 |
| 333 | N-cyclopropyl-2-(4-(2- methoxyphenyl)-6- methylnicotinamido)imidazo[2,1- b][1,3,4]thiadiazole-6-carboxamide | 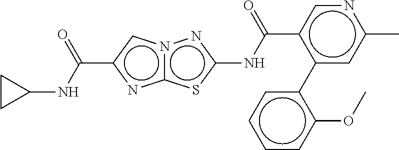 |
| 334 | 2′-chloro-5′-methoxy-6-methyl-N-(6- (spiro[2.3]hexan-5-yl)imidazo[2,1- b][1,3,4]thiadiazol-2-yl)-[4,4′- bipyridine]-3-carboxamide | 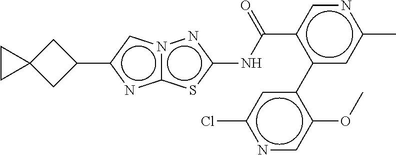 |
| 335 | 2′-chloro-N-(6- cyclopropylimidazo[2,1- b][1,3,4]thiadiazol-2-yl)-5′-methoxy- 6-methyl-[4,4′-bipyridine]-3- carboxamide | 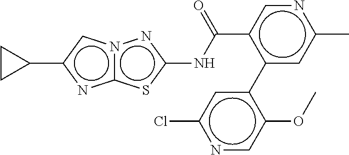 |
| 336 | N-(6-cyclopropylimidazo[2,1- b][1,3,4]thiadiazol-2-yl)-4-(2- methoxyphenyl)-6- methylnicotinamide | 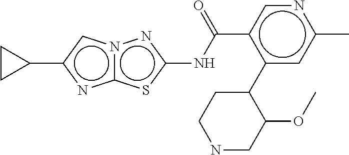 |
| 337 | 4-(5-cyano-2-methoxyphenyl)-N-(6- cyclopropylimidazo[2,1- b][1,3,4]thiadiazol-2-yl)-6- methylnicotinamide | 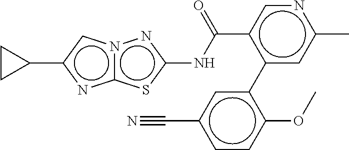 |
| 338 | 4-(2-methoxyphenyl)-6-methyl-N-(6- (pyridin-3-ylmethyl)imidazo[2,1- b][1,3,4]thiadiazol-2-yl)nicotinamide | 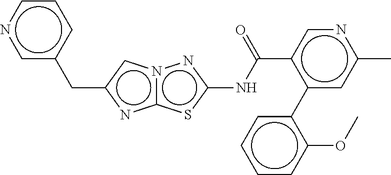 |
| 339 | 4-(2-methoxyphenyl)-6-methyl-N-(6- (pyridin-4-ylmethyl)imidazo[2,1- b][1,3,4]thiadiazol-2-yl)nicotinamide | 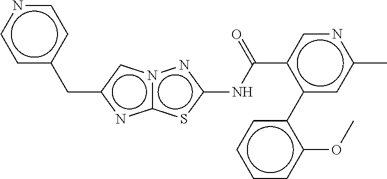 |
| 340 | (S or R)-4-(2-methoxyphenyl)-6- methyl-N-(6-(tetrahydrofuran-3- yl)imidazo[2,1-b][1,3,4]thiadiazol-2- yl)nicotinamide | 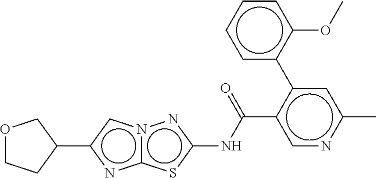 |
| 341 | 4-(2-methoxyphenyl)-6-methyl-N-(6- (pyridin-3-yl)imidazo[2,1- b][1,3,4]thiadiazol-2-yl)nicotinamide | 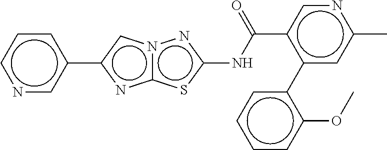 |
| 342 | 4-(2-methoxyphenyl)-6-methyl-N-(6- (pyridin-4-yl)imidazo[2,1- b][1,3,4]thiadiazol-2-yl)nicotinamide | 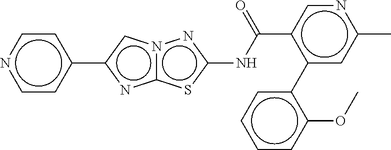 |
| 343 | (R or S)-4-(2-methoxyphenyl)-6- methyl-N-(6-((tetrahydrofuran-2- yl)methyl)imidazo[2,1- b][1,3,4]thiadiazol-2-yl)nicotinamide | 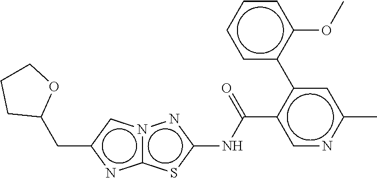 |
| 344 | (R or S)-4-(2-methoxyphenyl)-6- methyl-N-(6-(tetrahydrofuran-3- yl)imidazo[2,1-b][1,3,4]thiadiazol-2- yl)nicotinamide | 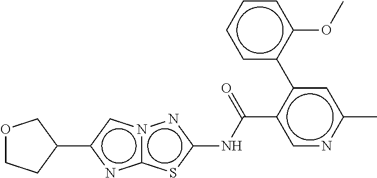 |
| 345 | 4-(2-methoxyphenyl)-6-methyl-N-(6- (thiazol-2-yl)imidazo[2,1- b][1,3,4]thiadiazol-2-yl)nicotinamide | 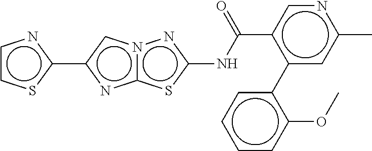 |
| 346 | N-(6-(1- cyanocyclopropyl)imidazo[2,1- b][1,3,4]thiadiazol-2-yl)-4-(2- methoxyphenyl)-6- methylnicotinamide | 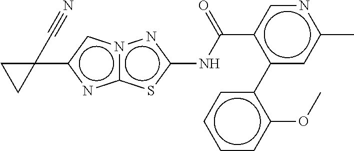 |
| 347 | N-(6-(tert-butyl)imidazo[2,1- b][1,3,4]thiadiazol-2-yl)-4-(2- methoxyphenyl)-6- methylnicotinamide | 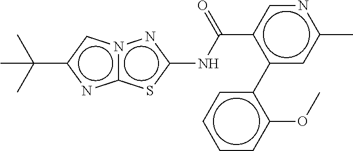 |
| 348 | 4-(2-methoxyphenyl)-6-methyl-N-(6- (pyrimidin-4-yl)imidazo[2,1- b][1,3,4]thiadiazol-2-yl)nicotinamide | 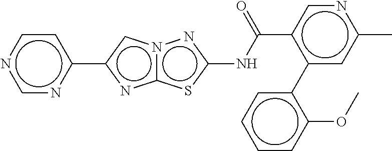 |
| 349 | (S or R)-4-(2-methoxyphenyl)-6- methyl-N-(6-((tetrahydrofuran-2- yl)methyl)imidazo[2,1- b][1,3,4]thiadiazol-2-yl)nicotinamide | 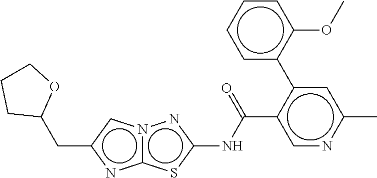 |
| 350 | 4-(2-methoxyphenyl)-6-methyl-N-(6- (pyridin-2-yl)imidazo[2,1- b][1,3,4]thiadiazol-2-yl)nicotinamide | 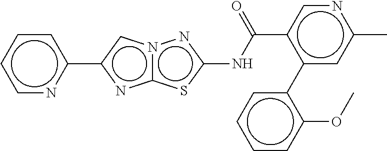 |
| 351 | 4-(2-methoxyphenyl)-6-methyl-N-(6- phenylimidazo[2,1- b][1,3,4]thiadiazol-2-yl)nicotinamide | 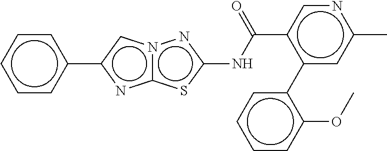 |
| 352 | (S or R)-4-(2-methoxyphenyl)-6- methyl-N-(6-((tetrahydrofuran-3- yl)methyl)imidazo[2,1- b][1,3,4]thiadiazol-2-yl)nicotinamide | 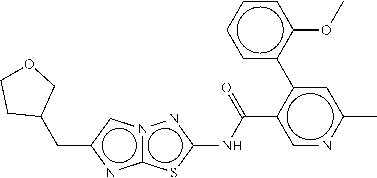 |
| 353 | (R or S)-4-(2-methoxyphenyl)-6- methyl-N-(6-((tetrahydrofuran-3- yl)methyl)imidazo[2,1- b][1,3,4]thiadiazol-2-yl)nicotinamide | 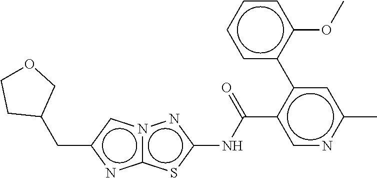 |
| 354 | 4-(2-methoxyphenyl)-6-methyl-N-(6- methylimidazo[2,1- b][1,3,4]thiadiazol-2-y1)nicotinamide | 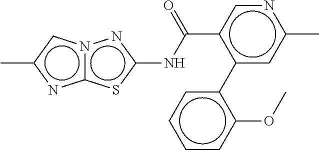 |
| 355 | 4-(2-methoxyphenyl)-6-methyl-N-(6- methyl-[1,2,4]triazolo[5,1- b][1,3,4]thiadiazol-2-yl)nicotinamide | 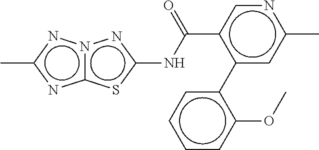 |
| 356 | tert-butyl 4-(2-methyl-5-((6- methylimidazo[2,1- b][1,3,4]thiadiazol-2- yl)carbamoyl)pyridin-4-yl)piperazine- 1-carboxylate | 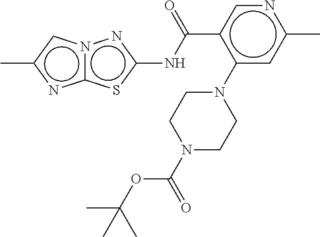 |
| 357 | 4-(2-methoxyphenyl)-6-methyl-N-(6- (pyridin-2-ylmethyl)imidazo[2,1- b][1,3,4]thiadiazol-2-yl)nicotinamide | 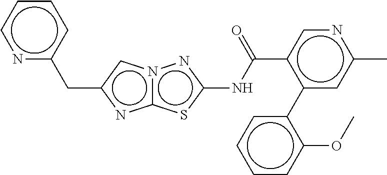 |
| 358 | (Racemic)-N-(6-(1-hydroxypropan-2- yl)imidazo[2,1-b][1,3,4]thiadiazol-2- yl)-4-(2-methoxyphenyl)-6- methylnicotinamide | 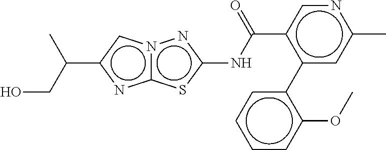 |

TABLE 4

| Compound No. | Name | Structure |
|---|---|---|
| 359 | 2′-chloro-N-(6-(4- cyanobicyclo[2.2.2]octan-1- yl)thiazolo[4,5-b]pyrazin-2- yl)-5′-methoxy-6-methyl- [4,4′-bipyridine]-3- carboxamide | 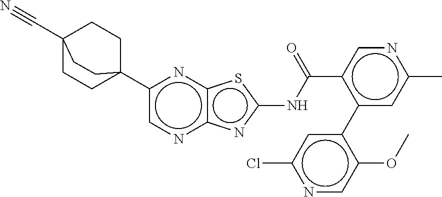 |
| 360 | 2′-chloro-N-(6-(3- cyanobicyclo[1.1.1]pentan- 1-yl)thiazolo[4,5-b]pyrazin- 2-yl)-5′-methoxy-6-methyl- [4,4′-bipyridine]-3- carboxamide | 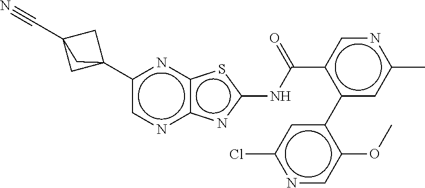 |
| 361 | 2′-chloro-N-(6-(5- cyanobicyclo[2.2.1 ]heptan- 2-yl)thiazolo[4,5-b]pyrazin- 2-yl)-5′-methoxy-6-methyl- [4,4′-bipyridine]-3- carboxamide | 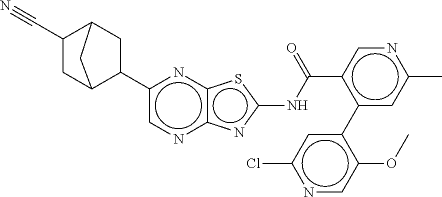 |
| 361A | 2′-chloro-N-(6-((2R or S,5R or S)-5- cyanobicyclo[2.2.1]heptan- 2-yl)thiazolo[4,5-b]pyrazin- 2-yl)-5′-methoxy-6-methyl- [4,4′-bipyridine]-3- carboxamide | 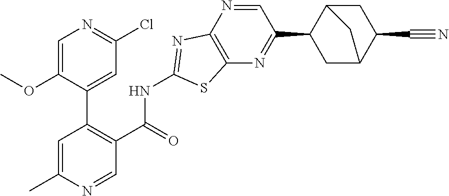 [SMILES: COc1cnc(Cl)cc1-c1cc(C)ncc1C(=O)Nc1nc2ncc([C@@H]3CC4CC3C[C@H]4C#N)nc2s1] |
| 361B | 2′-chloro-N-(6-((2S or R,5S or R)-5- cyanobicyclo[2.2.1 ]heptan- 2-yl)thiazolo[4,5-b]pyrazin- 2-yl)-5′-methoxy-6-methyl- [4,4′-bipyridine]-3- carboxamide | 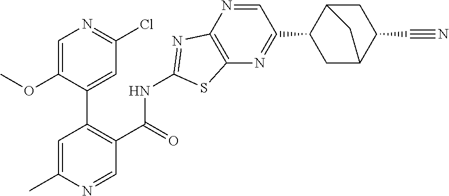 [SMILES: COc1cnc(Cl)cc1-c1cc(C)ncc1C(=O)Nc1nc2ncc([C@H]3CC4CC3C[C@@H]4C#N)nc2s1] |
| 361C | 2′-chloro-N-(6-((2R or S,5S or R)-5- cyanobicyclo[2.2.1 ]heptan- 2-yl)thiazolo[4,5-b]pyrazin- 2-yl)-5′-methoxy-6-methyl- [4,4′-bipyridine]-3- carboxamide | 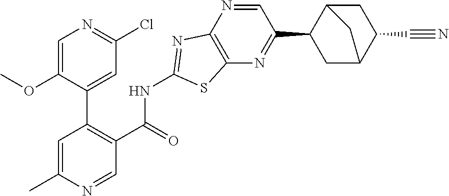 [SMILES: COc1cnc(Cl)cc1-c1cc(C)ncc1C(=O)Nc1nc2ncc([C@@H]3CC4CC3C[C@@H]4C#N)nc2s1] |
| 361D | 2′-chloro-N-(6-((2S or R,5R or S)-5- cyanobicyclo[2.2.1 ]heptan- 2-yl)thiazolo[4,5-b]pyrazin- 2-yl)-5′-methoxy-6-methyl- [4,4′-bipyridine]-3- carboxamide | 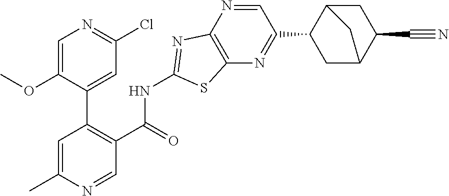 [SMILES: COc1cnc(Cl)cc1-c1cc(C)ncc1C(=O)Nc1nc2ncc([C@H]3CC4CC3C[C@H]4C#N)nc2s1] |
| 362 | 2′-chloro-N-(6-((3R or S,4S or R)-3-fluoro-4-methoxy- 4-methylpiperidin-1- yl)thiazolo[4,5-b]pyrazin-2- yl)-5′-methoxy-6-methyl- [4,4′-bipyridine]-3- carboxamide | 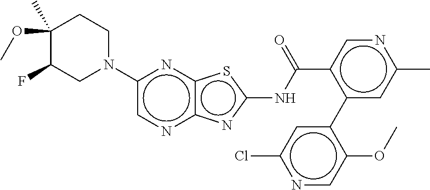 |
| 363 | 2′-chloro-N-(6-((3S or R,4R or S)-3-fluoro-4-methoxy-4- methylpiperidin-1- yl)thiazolo[4,5-b]pyrazin-2- yl)-5′-methoxy-6-methyl- [4,4′-bipyridine]-3- carboxamide | 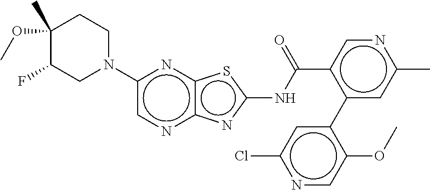 |
| 364 | (S or R)-2′-chloro-5′- methoxy-6-methyl-N-(6-(1- (2,2,2- trifluoroethyl)pyrrolidin-3- yl)thiazolo[4,5-b]pyrazin-2- yl)-[4,4′-bipyridine]-3- carboxamide | 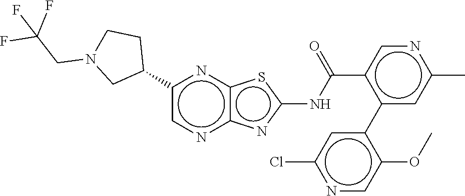 |
| 365 | (R or S)-2′-chloro-5′- methoxy-6-methyl-N-(6-(1- (2,2,2- trifluoroethyl)pyrrolidin-3- yl)thiazolo[4,5-b]pyrazin-2- yl)-[4,4′-bipyridine]-3- carboxamide | 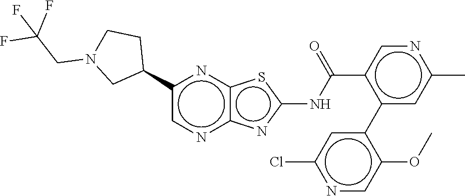 |
| 366 | (cis or trans)-2′-chloro-N- (6-(4-(difluoromethyl)-4- methoxycyclohexyl)thiazolo [4,5-b]pyrazin-2-yl)-5′- methoxy-6-methyl-[4,4′- bipyridine]-3-carboxamide | 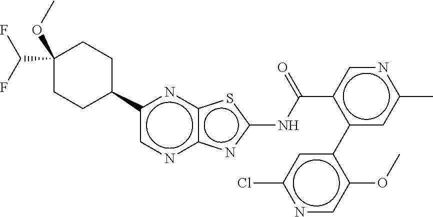 |
| 367 | (trans or cis)-2′-chloro-N- (6-(4-(difluoromethyl)-4- methoxycyclohexyl)thiazolo [4,5-b]pyrazin-2-yl)-5′- methoxy-6-methyl-[4,4′- bipyridine]-3-carboxamide | 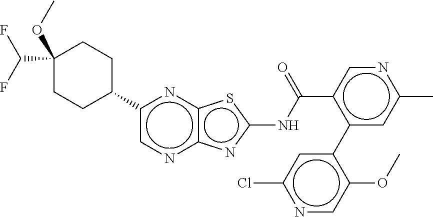 |
| 368 | (trans or cis)-N-(6-(4- cyanocyclohexyl)thiazolo[4, 5-b]pyrazin-2-yl)-4-(6- (difluoromethyl)-3- methoxypyridazin-4-yl)-6- methylnicotinamide | 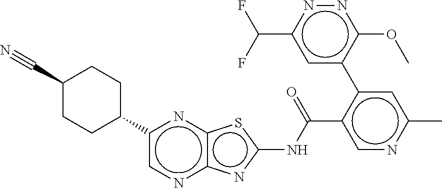 |
| 369 | 2′-chloro-5′-methoxy-N-(6- ((trans or cis)-4-methoxy-4- methylcyclohexyl)thiazolo[ 4,5-b]pyrazin-2-yl)-6- methyl-[4,4′-bipyridine]-3- carboxamide | 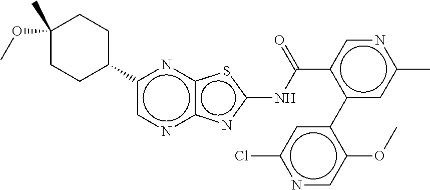 |
| 370 | (cis or trans)-4-(6- (difluoromethyl)-3- methoxypyridazin-4-yl)-N- (6-(4-hydroxy-4- methylcyclohexyl)thiazolo[ 4,5-b]pyrazin-2-yl)-6- methylnicotinamide | 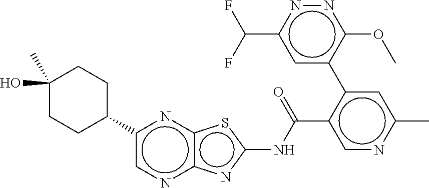 |
| 371 | (trans or cis)-4-(6- (difluoromethyl)-3- methoxypyridazin-4-yl)-N- (6-(4-hydroxy-4- methylcyclohexyl)thiazolo[ 4,5-b]pyrazin-2-yl)-6- methylnicotinamide | 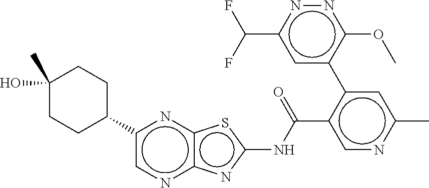 |
| 372 | (cis or trans)-N-(6-(4- cyanocyclohexyl)thiazolo[4, 5-b]pyrazin-2-yl)-4-(6- (difluoromethyl)-3- methoxypyridazin-4-yl)-6- methylnicotinamide | 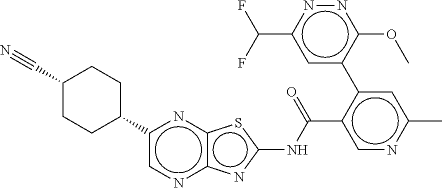 |
| 373 | 2′-chloro-5′-methoxy-N-(6- (4-methoxytetrahydro-2H- pyran-4-yl)thiazolo[4,5- b]pyrazin-2-yl)-6-methyl- [4,4′-bipyridine]-3- carboxamide | 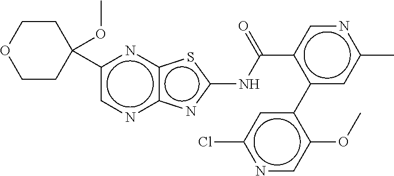 |
| 374 | (cis or trans)-2′-chloro-N- (6-(4-(difluoromethyl)-4- hydroxycyclohexyl)thiazolo [4,5-b]pyrazin-2-yl)-5′- methoxy-6-methyl-[4,4′- bipyridine]-3-carboxamide | 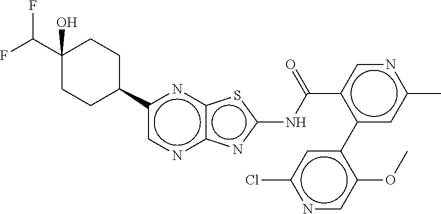 |
| 375 | 2′-chloro-N-(6-((3R or S,4S or R)-3-fluoro-4-hydroxy-4- methylpiperidin-1- yl)thiazolo[4,5-b]pyrazin-2- yl)-5′-methoxy-6-methyl- [4,4′-bipyridine]-3- carboxamide | 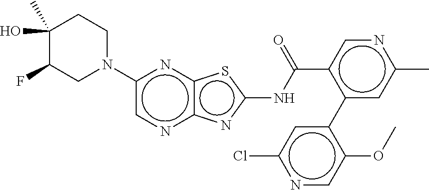 |
| 376 | 2′-chloro-N-(6-((3S or R,4R or S)-3-fluoro-4-hydroxy-4- methylpiperidin-1- yl)thiazolo[4,5-b]pyrazin-2- yl)-5′-methoxy-6-methyl- [4,4′-bipyridine]-3- carboxamide | 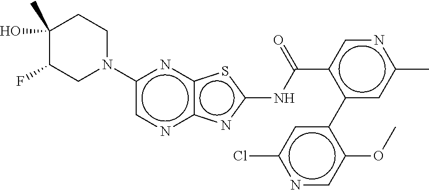 |
| 377 | 2′-chloro-5′-methoxy-N-(6- ((trans or cis)-4-methoxy-4- (trifluoromethyl)cyclohexyl) thiazolo[4,5-b]pyrazin-2- yl)-6-methyl-[4,4′- bipyridine]-3-carboxamide | 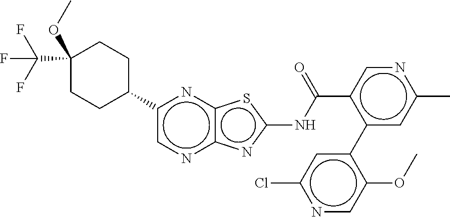 |
| 378 | 2′-chloro-5′-methoxy-6- methyl-N-(6-(1-(2,2,2- trifluoroethyl)azetidin-3- yl)thiazolo[4,5-b]pyrazin-2- yl)-[4,4′-bipyridine]-3- carboxamide | 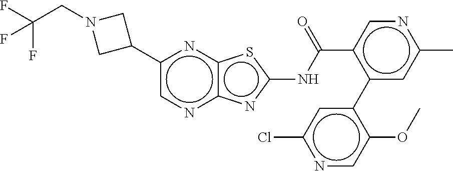 |
| 379 | (S or R)-2′-chloro-N-(6- (2,2-dimethyltetrahydro-2H- pyran-4-yl)thiazolo[4,5- b]pyrazin-2-yl)-5′-methoxy- 6-methyl-[4,4′-bipyridine]- 3-carboxamide | 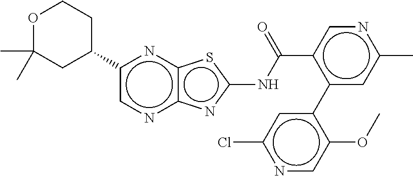 |
| 380 | 2′-chloro-5′-methoxy-N-(6- ((cis or trans)-4-methoxy-4- (trifluoromethyl)cyclohexyl) thiazolo[4,5-b]pyrazin-2- yl)-6-methyl-[4,4′- bipyridine]-3-carboxamide | 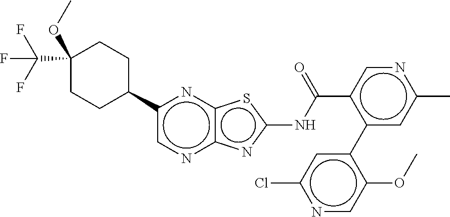 |
| 381 | (trans or cis) 2′-chloro-N-(6- (4-(difluoromethyl)-4- hydroxycyclohexyl)thiazolo [4,5-b]pyrazin-2-yl)-5′- methoxy-6-methyl-[4,4′- bipyridine]-3-carboxamide | 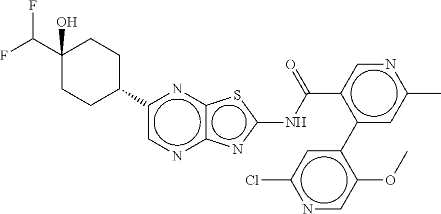 |
| 382 | (R or S)-2′-chloro-N-(6- (2,2-dimethyltetrahydro-2H- pyran-4-yl)thiazolo[4,5- b]pyrazin-2-yl)-5′-methoxy- 6-methyl-[4,4′-bipyridine]- 3-carboxamide | 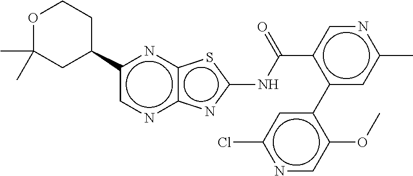 |
| 383 | 2′-chloro-N-(6-(4- (difluoromethyl)-4- methoxycyclohexyl)thiazolo [4,5-b]pyrazin-2-yl)-5′- methoxy-6-methyl-[4,4′- bipyridine]-3-carboxamide | 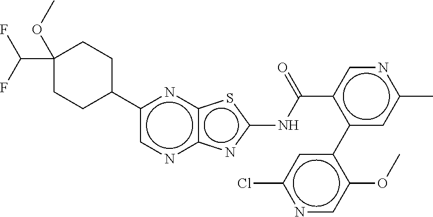 |
| 384 | 4-(6-(difluoromethyl)-3- methoxypyridazin-4-yl)-6- methyl-N-(6-(2- methylpyridin-4- yl)thiazolo[4,5-b]pyrazin-2- yl)nicotinamide | 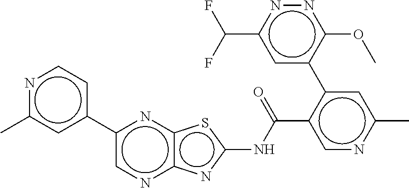 |
| 385 | 2′-chloro-5′-methoxy-N-(6- ((cis or trans)-4-methoxy-4- methylcyclohexyl)thiazolo[ 4,5-b]pyrazin-2-yl)-6- methyl-[4,4′-bipyridine]-3- carboxamide | 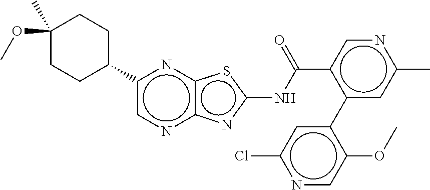 |
| 386 | (cis or trans)-2′-chloro-N- (6-(4-cyano-4- methylcyclohexyl)thiazolo[ 4,5-b]pyrazin-2-yl)-5′- methoxy-6-methyl-[4,4′- bipyridine]-3-carboxamide | 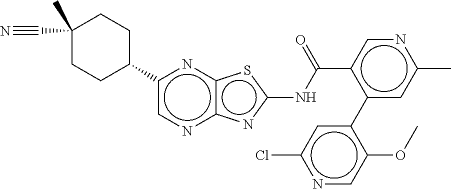 |
| 387 | (cis or trans)-2′-chloro-N- (6-(4-(difluoromethyl)-4- methoxycyclohexyl)thiazolo [4,5-b]pyrazin-2-yl)-5′- methoxy-6-methyl-[4,4′- bipyridine]-3-carboxamide | 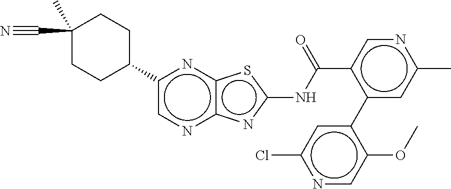 |
| 388 | 2′-chloro-N-(6-(4- (difluoromethyl)-4- methoxycyclohex-1-en-1- yl)thiazolo[4,5-b]pyrazin-2- yl)-5′-methoxy-6-methyl- [4,4′-bipyridine]-3- carboxamide | 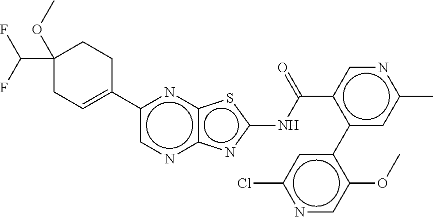 |
| 389 | 2′-chloro-N-(6-(2,2- dimethyltetrahydro-2H- pyran-4-yl)thiazolo[4,5- b]pyrazin-2-yl)-5′-methoxy- 6-methyl-[4,4′-bipyridine]- 3-carboxamide | 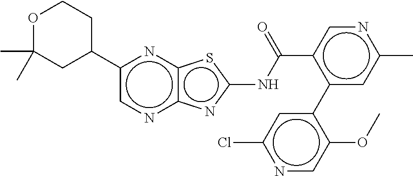 |
| 390 | 2′-chloro-N-(6-(4- hydroxytetrahydro-2H- pyran-4-yl)thiazolo[4,5- b]pyrazin-2-yl)-5′-methoxy- 6-methyl-[4,4′-bipyridine]- 3-carboxamide | 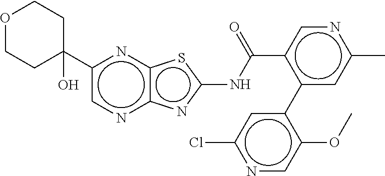 |
| 391 | 2′-chloro-N-(6-((cis or trans)-4- (difluoromethoxy)cyclohexyl) thiazolo[4,5-b]pyrazin-2- yl)-5′-methoxy-6-methyl- [4,4′-bipyridine]-3- carboxamide | 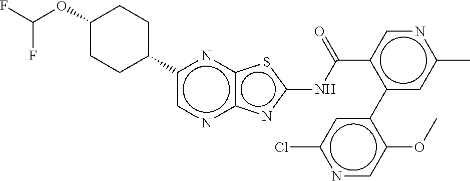 |
| 392 | 2′-chloro-N-(6-((trans or cis)-4- (difluoromethoxy)cyclohexyl) thiazolo[4,5-b]pyrazin-2- yl)-5′-methoxy-6-methyl- [4,4′-bipyridine]-3- carboxamide | 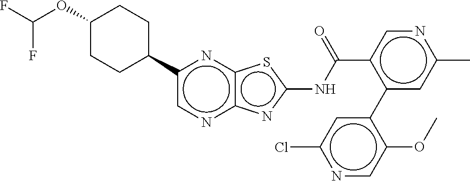 |
| 393 | 2′-chloro-N-(6-((4aS,7aS)- hexahydrocyclopenta[b][1,4] oxazin-4(4aH)- yl)thiazolo[4,5-b]pyrazin-2- yl)-5′-methoxy-6-methyl- [4,4′-bipyridine]-3- carboxamide | 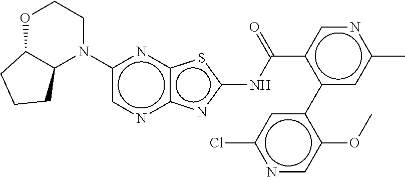 |
| 394 | 2′-chloro-N-(6-((4aR,7aR)- hexahydrocyclopenta[b][1,4] oxazin-4(4aHI)- yl)thiazolo[4,5-b]pyrazin-2- yl)-5′-methoxy-6-methyl- [4,4′-bipyridine]-3- carboxamide | 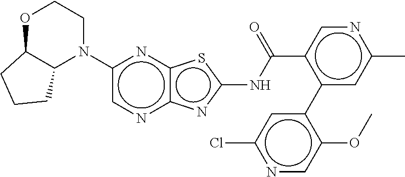 |
| 395 | 2′-chloro-N-(6-((4aS,7aR)- hexahydrocyclopenta[b][1,4] oxazin-4(4aH)- yl)thiazolo[4,5-b]pyrazin-2- yl)-5′-methoxy-6-methyl- [4,4′-bipyridine]-3- carboxamide | 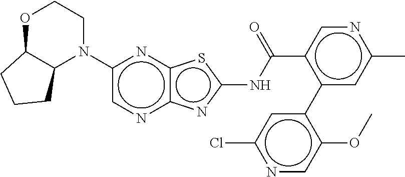 |
| 396 | 2′-chloro-N-(6-(4- (difluoromethyl)-4- hydroxycyclohex-1-en-1- yl)thiazolo[4,5-b]pyrazin-2- yl)-5′-methoxy-6-methyl- [4,4′-bipyridine]-3- carboxamide | 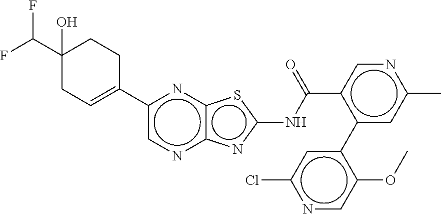 |
| 397 | 2′-chloro-N-(6-((4aR,7aS)- hexahydrocyclopenta[b][1,4] oxazin-4(4aH)- yl)thiazolo[4,5-b]pyrazin-2- yl)-5′-methoxy-6-methyl- [4,4′-bipyridine]-3- carboxamide | 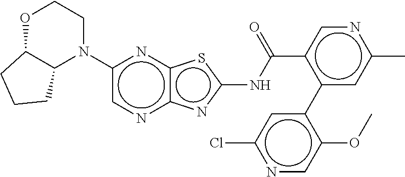 |
| 398 | 2′-chloro-N-(6-(4- (difluoromethoxy)cyclohexyl) thiazolo[4,5-b]pyrazin-2- yl)-5′-methoxy-6-methyl- [4,4′-bipyridine]-3- carboxamide | 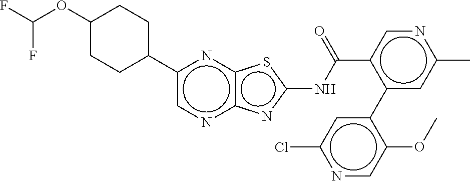 |
| 399 | (cis or trans)-2′-chloro-N- (6-(4-hydroxy-4- (trifluoromethyl)cyclohexyl) thiazolo[4,5-b]pyrazin-2- yl)-5′-methoxy-6-methyl- [4,4′-bipyridine]-3- carboxamide | 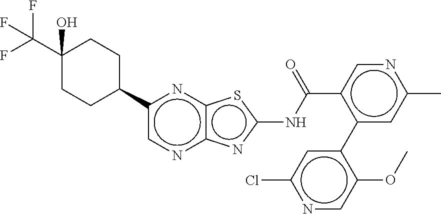 |
| 400 | 2′-chloro-N-(6-((2R or S,3S or R)-2,3- dimethylmorpholino)thiazolo [4,5-b]pyrazin-2-yl)-5′- methoxy-6-methyl-[4,4′- bipyridine]-3-carboxamide | 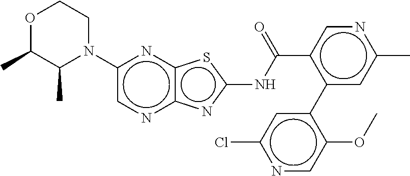 |
| 401 | (trans or cis)-2′-chloro-N- (6-(4-hydroxy-4- (trifluoromethyl)cyclohexyl) thiazolo[4,5-b]pyrazin-2- yl)-5′-methoxy-6-methyl- [4,4′-bipyridine]-3- carboxamide | 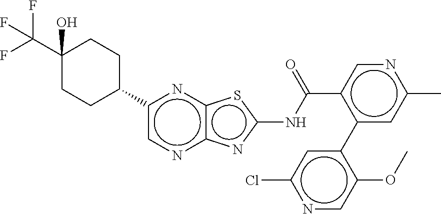 |
| 402 | 2′-chloro-N-(6-((2R or S,3R or S)-2,3- dimethylmorpholino)thiazolo [4,5-b]pyrazin-2-yl)-5′- methoxy-6-methyl-[4,4′- bipyridine]-3-carboxamide | 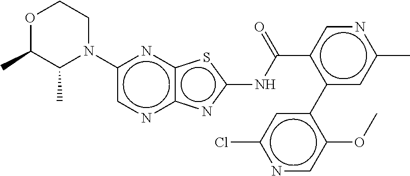 |
| 403 | 2′-chloro-5′-methoxy-6- methyl-N-(6-((trans or cis)- 4- (trifluoromethoxy)cyclohexyl) thiazolo[4,5-b]pyrazin-2- yl)-[4,4′-bipyridine]-3- carboxamide | 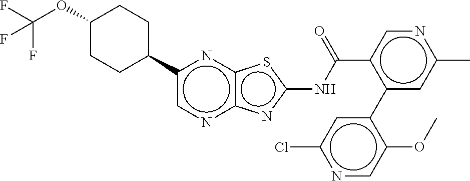 |
| 404 | 2′-chloro-N-(6-((3R,5S)- 3,5- dimethylmorpholino)thiazolo [4,5-b]pyrazin-2-yl)-5′- methoxy-6-methyl-[4,4′- bipyridine]-3-carboxamide | 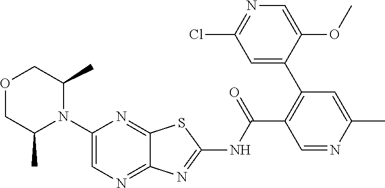 [SMILES: COc1cnc(Cl)cc1-c1cc(C)ncc1C(=O)Nc1nc2ncc(N3[C@H](C)COC[C@@H]3C)nc2s1] |
| 405 | 2′-chloro-N-(6-(4- (difluoromethoxy)cyclohex- 1-en-1-yl)thiazolo[4,5- b]pyrazin-2-yl)-5′-methoxy- 6-methyl-[4,4′-bipyridine]- 3-carboxamide | 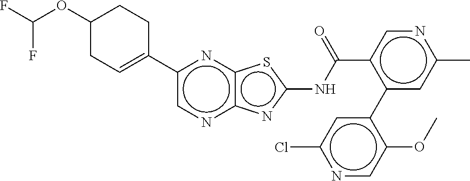 |
| 406 | 2′-chloro-5′-methoxy-6- methyl-N-(6-((cis or trans)- 4- (trifluoromethoxy)cyclohexyl) thiazolo[4,5-b]pyrazin-2- yl)-[4,4′-bipyridine]-3- carboxamide | 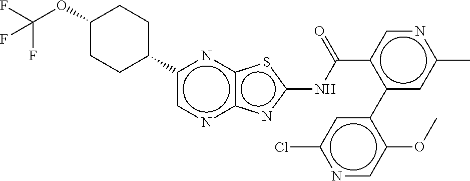 |
| 407 | 2′-chloro-N-(6-(4-cyano-4- methylcyclohex-1-en-1- yl)thiazolo[4,5-b]pyrazin-2- yl)-5′-methoxy-6-methyl- [4,4′-bipyridine]-3- carboxamide | 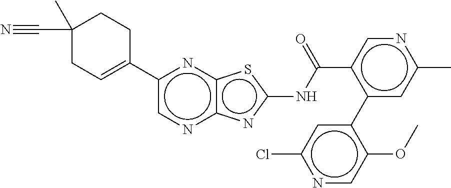 |
| 408 | 2′-chloro-N-(6-((2S or R,3R or S)-2,3- dimethylmorpholino)thiazolo [4,5-b]pyrazin-2-yl)-5′- methoxy-6-methyl-[4,4′- bipyridine]-3-carboxamide | 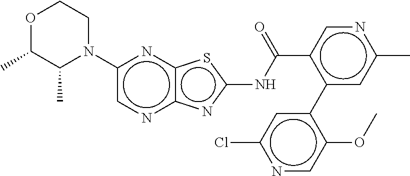 |
| 409 | 2′-chloro-N-(6-((2R,5R)- 2,5- dimethylmorpholino)thiazolo [4,5-b]pyrazin-2-yl)-5′- methoxy-6-methyl-[4,4′- bipyridine]-3-carboxamide | 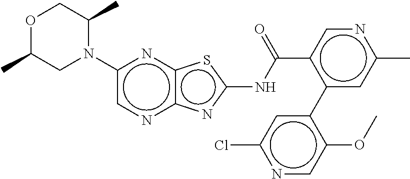 |
| 410 | 2′-chloro-N-(6-(4-cyano-4- methylcyclohexyl)thiazolo[ 4,5-b]pyrazin-2-yl)-5′- methoxy-6-methyl-[4,4′- bipyridine]-3-carboxamide | 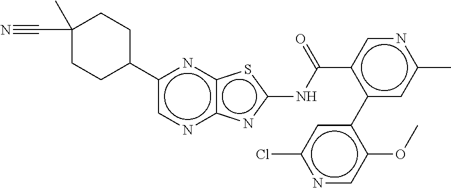 |
| 411 | (cis or trans)-4-(5-cyano-2- methoxyphenyl)-N-(6-(4- (hydroxymethyl)cyclohexyl) thiazolo[4,5-b]pyrazin-2- yl)-6-methylnicotinamide | 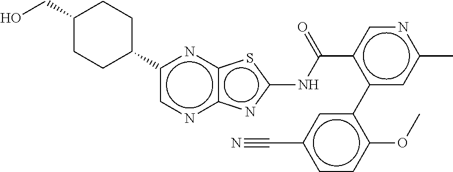 |
| 412 | 2′-chloro-N-(6-((2S,5S)-2,5- dimethylmorpholino)thiazolo [4,5-b]pyrazin-2-yl)-5′- methoxy-6-methyl-[4,4′- bipyridine]-3-carboxamide | 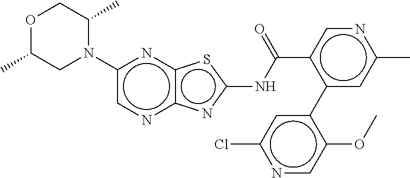 |
| 413 | 2′-chloro-N-(6-(4-hydroxy- 4- (trifluoromethyl)cyclohex- 1-en-1-yl)thiazolo[4,5- b]pyrazin-2-yl)-5′-methoxy- 6-methyl-[4,4′-bipyridine]- 3-carboxamide | 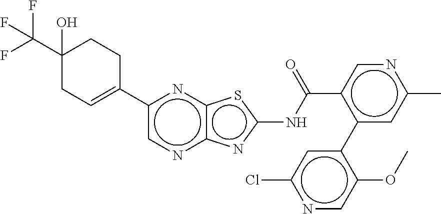 |
| 414 | (R)-2′-chloro-5′-methoxy-6- methyl-N-(6-(3- (trifluoromethyl)morpholino) thiazolo[4,5-b]pyrazin-2- yl)-[4,4′-bipyridine]-3- carboxamide | 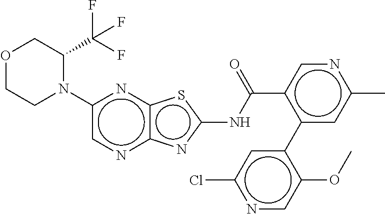 |
| 415 | 2′-chloro-N-(6-((2S or R,3S or R)-2,3- dimethylmorpholino)thiazolo [4,5-b]pyrazin-2-yl)-5′- methoxy-6-methyl-[4,4′- bipyridine]-3-carboxamide | 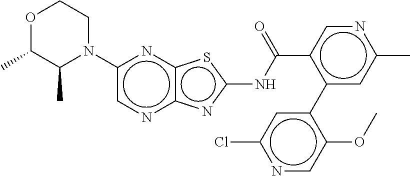 |
| 416 | (S)-2′-chloro-5′-methoxy-6- methyl-N-(6-(3- (trifluoromethyl)morpholino) thiazolo[4,5-b]pyrazin-2- yl)-[4,4′-bipyridine]-3- carboxamide | 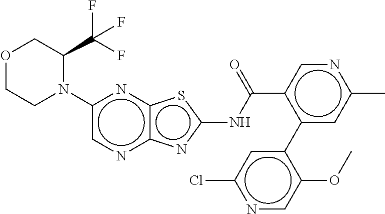 |
| 417 | (trans or cis)-4-(5-cyano-2- methoxyphenyl)-N-(6-(4- (hydroxymethyl)cyclohexyl) thiazolo[4,5-b]pyrazin-2- yl)-6-methylnicotinamide | 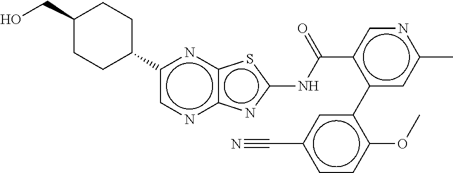 |
| 418 | 4-(5-cyano-2- methoxyphenyl)-N-(6-(4- (difluoromethyl)-4- hydroxypiperidin-1- yl)thiazolo[4,5-b]pyrazin-2- yl)-6-methylnicotinamide | 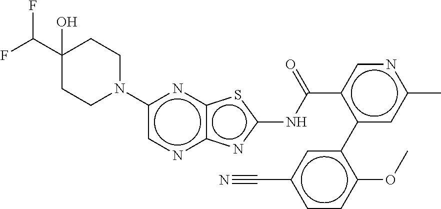 |
| 419 | 2′-chloro-N-(6-((2R,5S)- 2,5- dimethylmorpholino)thiazolo [4,5-b]pyrazin-2-yl)-5′- methoxy-6-methyl-[4,4′- bipyridine]-3-carboxamide | 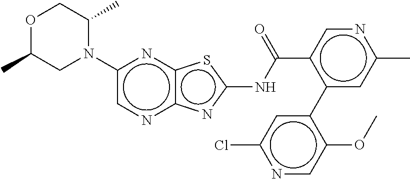 |
| 420 | 2′-chloro-5′-methoxy-6- methyl-N-(6-(4- (trifluoromethoxy)cyclohex- 1-en-1-yl)thiazolo[4,5- b]pyrazin-2-yl)-[4,4′- bipyridine]-3-carboxamide | 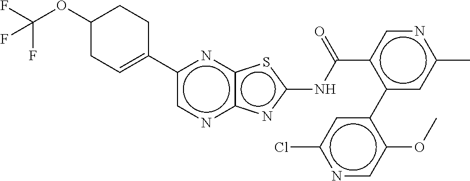 |
| 421 | 4-(5-cyano-2- methoxyphenyl)-N-(6-(4- (hydroxymethyl)cyclohex- 1-en-1-yl)thiazolo[4,5- b]pyrazin-2-yl)-6- methylnicotinamide | 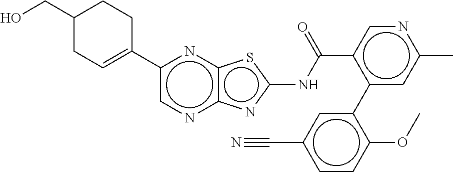 |
| 422 | 2′-chloro-N-(6-((2S,5R)- 2,5- dimethylmorpholino)thiazolo [4,5-b]pyrazin-2-yl)-5′- methoxy-6-methyl-[4,4′- bipyridine]-3-carboxamide | 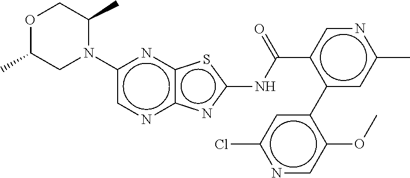 |
| 423 | 2′-chloro-N-(6-((3R,5R)- 3,5- dimethylmorpholino)thiazolo [4,5-b]pyrazin-2-yl)-5′- methoxy-6-methyl-[4,4′- bipyridine]-3-carboxamide | 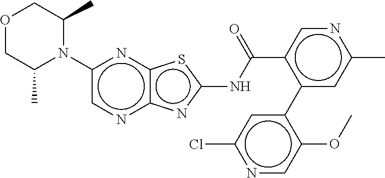 |
| 424 | 2′-chloro-N-(6- (hexahydrocyclopenta[b][1, 4]oxazin-4(4aH)- yl)thiazolo[4,5-b]pyrazin-2- yl)-5′-methoxy-6-methyl- [4,4′-bipyridine]-3- carboxamide | 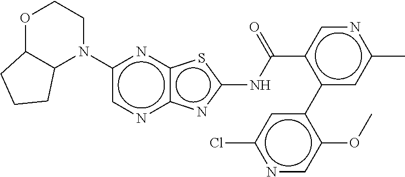 |
| 425 | 2′-chloro-N-(6-(2,3- dimethylmorpholino)thiazolo [4,5-b]pyrazin-2-yl)-5′- methoxy-6-methyl-[4,4′- bipyridine]-3-carboxamide | 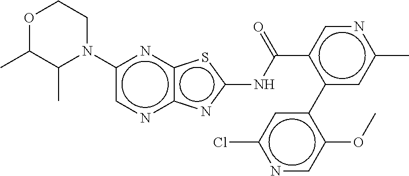 |
| 426 | 2′-chloro-N-(6-(3- (difluoromethyl)morpholino) thiazolo[4,5-b]pyrazin-2- yl)-5′-methoxy-6-methyl- [4,4′-bipyridine]-3- carboxamide | 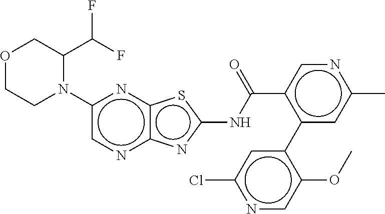 |
| 427 | (trans or cis)-2′-chloro-N- (6-(4-(2-hydroxypropan-2- yl)cyclohexyl)thiazolo[4,5- b]pyrazin-2-yl)-5′-methoxy- 6-methyl-[4,4′-bipyridine]- 3-carboxamide | 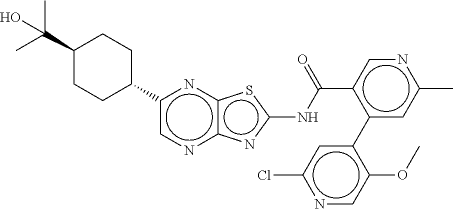 |
| 428 | (cis or trans)-2′-chloro-N- (6-(4-(2-hydroxypropan-2- yl)cyclohexyl)thiazolo[4,5- b]pyrazin-2-yl)-5′-methoxy- 6-methyl-[4,4′-bipyridine]- 3-carboxamide | 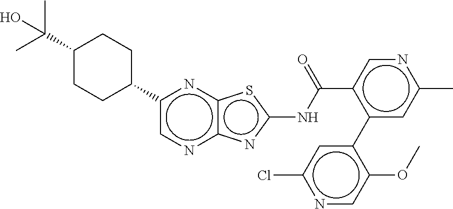 |
| 429 | 2′-chloro-N-(6-(2- (difluoromethyl)morpholino) thiazolo[4,5-b]pyrazin-2- yl)-5′-methoxy-6-methyl- [4,4′-bipyridine]-3- carboxamide | 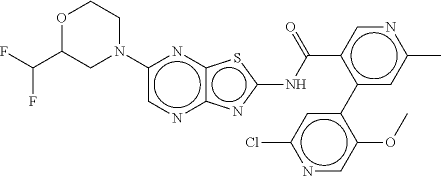 |
| 430 | (S)-2′-chloro-5′-methoxy-6- methyl-N-(6-(2- (trifluoromethyl)morpholino) thiazolo[4,5-b]pyrazin-2- yl)-[4,4′-bipyridine]-3- carboxamide | 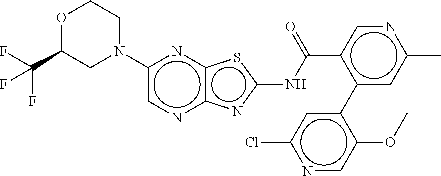 |
| 431 | 2′-chloro-5′-methoxy-6- methyl-N-(6-((2R or S,4S or R)-2-methyltetrahydro-2H- pyran-4-yl)thiazolo[4,5- b]pyrazin-2-yl)-[4,4′- bipyridine]-3-carboxamide | 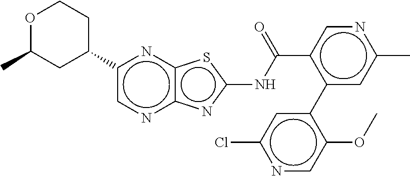 |
| 432 | 2′-chloro-5′-methoxy-6- methyl-N-(6-(1-(2,2,2- trifluoroethyl)piperidin-4- yl)thiazolo[4,5-b]pyrazin-2- yl)-[4,4′-bipyridine]-3- carboxamide | 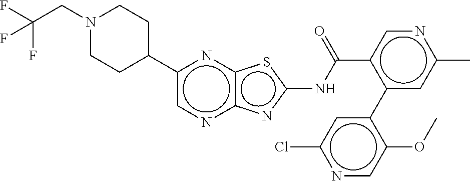 |
| 433 | 2′-chloro-5′-methoxy-6- methyl-N-(6-((2S,4R)-2- methyltetrahydro-2H-pyran- 4-yl)thiazolo[4,5-b]pyrazin- 2-yl)-[4,4′-bipyridine]-3- carboxamide | 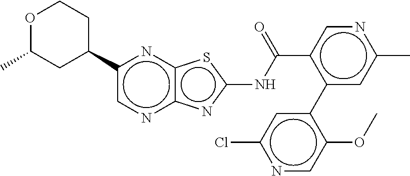 |
| 434 | 2′-chloro-5′-methoxy-6- methyl-N-(6-((2S or R,4R or S)-2-methyltetrahydro- 2H-pyran-4-yl)thiazolo[4,5- b]pyrazin-2-yl)-[4,4′- bipyridine]-3-carboxamide | 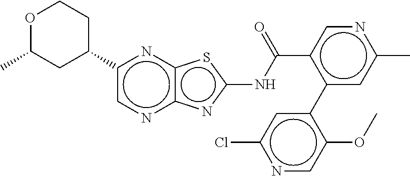 |
| 434A | 2′-chloro-5′-methoxy-6- methyl-N-(6-((2S or R,4S or R)-2-methyltetrahydro-2H- pyran-4-yl)thiazolo[4,5- b]pyrazin-2-yl)-[4,4′- bipyridine]-3-carboxamide | 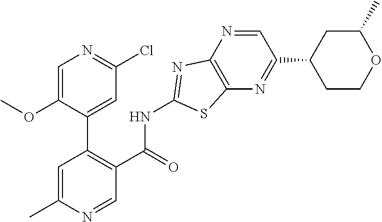 |
| 435 | 2′-chloro-5′-methoxy-6- methyl-N-(6-((2R or S,4R or S)-2-methyltetrahydro- 2H-pyran-4-yl)thiazolo[4,5- b]pyrazin-2-yl)-[4,4′- bipyridine]-3-carboxamide | 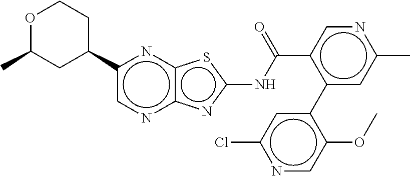 |
| 436 | 2′-chloro-N-(6-((2S,6S)-2,6- dimethylmorpholino)thiazolo [4,5-b]pyrazin-2-yl)-5′- methoxy-6-methyl-[4,4′- bipyridine]-3-carboxamide | 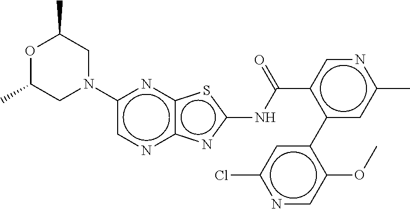 |
| 437 | 2′-chloro-N-(6-((2S,6R)- 2,6- dimethylmorpholino)thiazolo [4,5-b]pyrazin-2-yl)-5′- methoxy-6-methyl-[4,4′- bipyridine]-3-carboxamide | 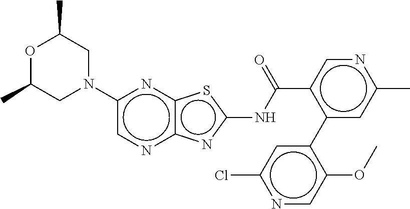 |
| 438 | (trans or cis)-4-(2- methoxyphenyl)-6-methyl- N-(6-(tetrahydrofuran-3- yl)imidazo[2,1- b][1,3,4]thiadiazol-2- yl)nicotinamide | 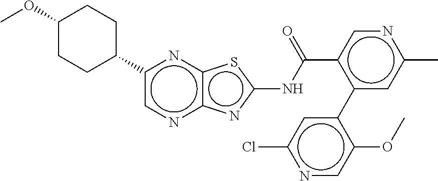 |
| 439 | (cis or trans)-2′-chloro-N- (6-(4-hydroxy-4- methylcyclohexyl)thiazolo[ 4,5-b]pyrazin-2-yl)-5′- methoxy-6-methyl-[4,4′- bipyridine]-3-carboxamide | 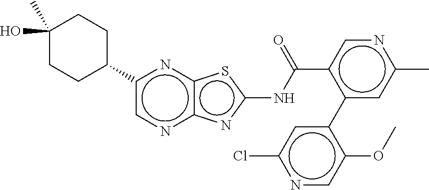 |
| 440 | (R)-2′-chloro-5′-methoxy-6- methyl-N-(6-(2- (trifluoromethyl)morpholino) thiazolo[4,5-b]pyrazin-2- yl)-[4,4′-bipyridine]-3- carboxamide | 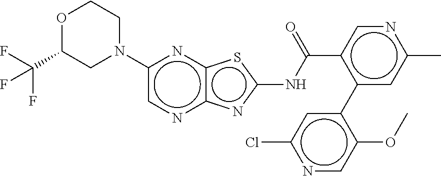 |
| 441 | (S or R)-2′-chloro-N-(6- (3,3-difluoro-4- methoxypiperidin-1- yl)thiazolo[4,5-b]pyrazin-2- yl)-5′-methoxy-6-methyl- [4,4′-bipyridine]-3- carboxamide | 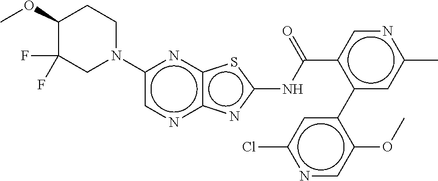 |
| 442 | (trans or cis)-2′-chloro-N- (6-(4-hydroxy-4- methylcyclohexyl)thiazolo[ 4,5-b]pyrazin-2-yl)-5′- methoxy-6-methyl-[4,4′- bipyridine]-3-carboxamide | 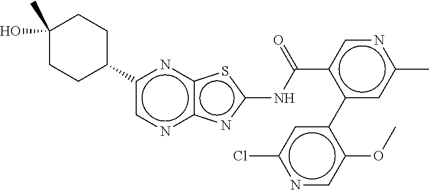 |
| 443 | 2′-chloro-5′-methoxy-6- methyl-N-(6-(2-methyl-3,6- dihydro-2H-pyran-4- yl)thiazolo[4,5-b]pyrazin-2- yl)-[4,4′-bipyridine]-3- carboxamide | 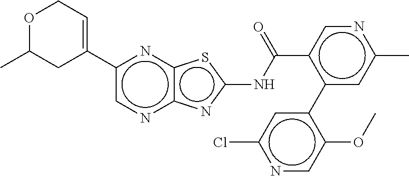 |
| 444 | (R or S)-2′-chloro-N-(6- (3,3-difluoro-4- methoxypiperidin-1- yl)thiazolo[4,5-b]pyrazin-2- yl)-5′-methoxy-6-methyl- [4,4′-bipyridine]-3- carboxamide | 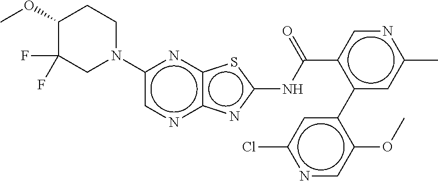 |
| 445 | 2′-chloro-6-cyclopropyl-5′- methoxy-N-(6-(2- methylpyridin-4- yl)thiazolo[4,5-b]pyrazin-2- yl)-[4,4′-bipyridine]-3- carboxamide | 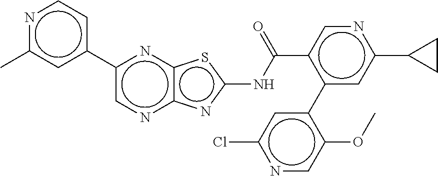 |
| 447 | (cis or trans)-4-(2- methoxyphenyl)-6-methyl- N-(6-(tetrahydrofuran-3- yl)imidazo[2,1- b][1,3,4]thiadiazol-2- yl)nicotinamide | 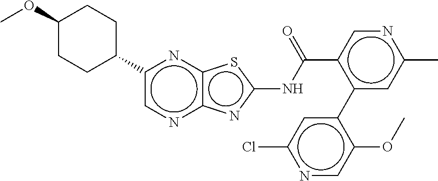 |
| 449 | 2′-chloro-5′-methoxy-N-[6- (4-methoxycyclohex-1-en- 1-yl)-[1,3]thiazolo[4,5- b]pyrazin-2-yl]-6-methyl- [4,4′-bipyridine]-3- carboxamide | 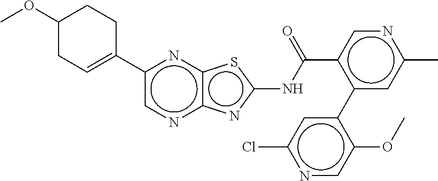 |
| 450 | 2′-chloro-N-(6-((3S,4R)-3- fluoro-4-methoxypiperidin- 1-yl)thiazolo[4,5-b]pyrazin- 2-yl)-5′-methoxy-6-methyl- [4,4′-bipyridine]-3- carboxamide | 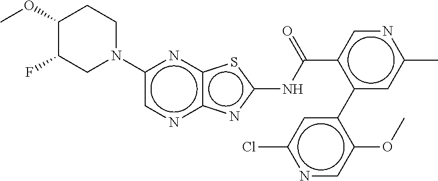 |
| 451 | 2′-chloro-N-(6-((1s,4s)-4- cyanocyclohexyl)thiazolo[4, 5-b]pyrazin-2-yl)-5′- methoxy-6-methyl-[4,4′- bipyridine]-3-carboxamide | 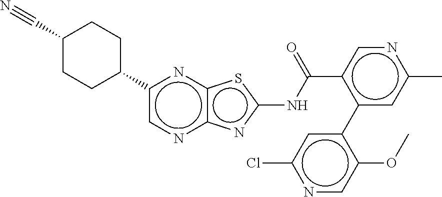 |
| 451A | (cis or trans)-4-(5-cyano-2- methoxyphenyl)-N-(6-(4- cyanocyclohexyl)thiazolo[4, 5-b]pyrazin-2-yl)-6- methylnicotinamide | 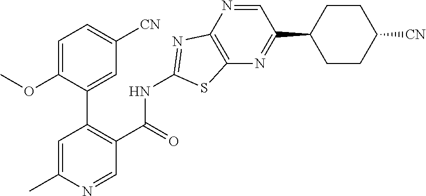 [SMILES: COc1ccc(C#N)cc1-c1cc(C)ncc1C(=O)Nc1nc2ncc([C@H]3CC[C@H](C#N)CC3)nc2s1] |
| 452 | 2′-chloro-N-(6-((3S,4S)-3- fluoro-4-methoxypiperidin- 1-yl)thiazolo[4,5-b]pyrazin- 2-yl)-5′-methoxy-6-methyl- [4,4′-bipyridine]-3- carboxamide | 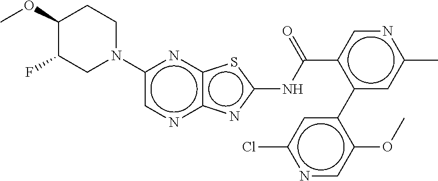 |
| 453 | 2′-chloro-N-(6-((3R,4S)-3- fluoro-4-methoxypiperidin- 1-yl)thiazolo[4,5-b]pyrazin- 2-yl)-5′-methoxy-6-methyl- [4,4′-bipyridine]-3- carboxamide | 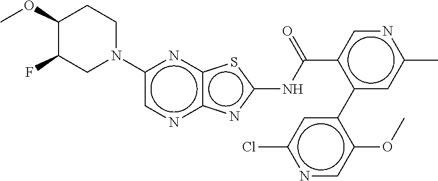 |
| 454 | 2′-chloro-N-(6-((1r,4r)-4- cyanocyclohexyl)thiazolo[4, 5-b]pyrazin-2-yl)-5′- methoxy-6-methyl-[4,4′- bipyridine]-3-carboxamide | 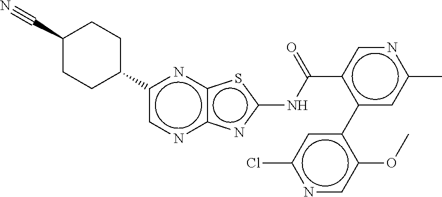 |
| 456 | 2′-chloro-N-(6-((3R,4R)-3- fluoro-4-methoxypiperidin- 1-yl)thiazolo[4,5-b]pyrazin- 2-yl)-5′-methoxy-6-methyl- [4,4′-bipyridine]-3- carboxamide | 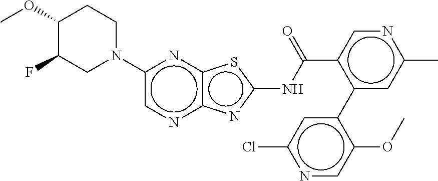 |
| 457 | (S or R)-2′-chloro-N-(6- (3,3-difluoro-4- hydroxypiperidin-1- yl)thiazolo[4,5-b]pyrazin-2- yl)-5′-methoxy-6-methyl- [4,4′-bipyridine]-3- carboxamide | 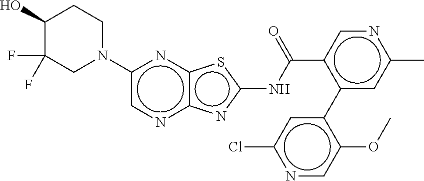 |
| 458 | (R or S)-2′-chloro-N-(6- (3,3-difluoro-4- hydroxypiperidin-1- yl)thiazolo[4,5-b]pyrazin-2- yl)-5′-methoxy-6-methyl- [4,4′-bipyridine]-3- carboxamide | 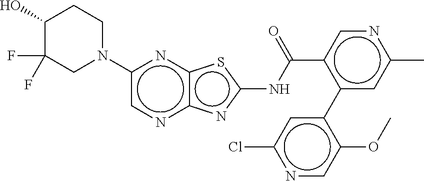 |
| 460 | 2′-chloro-N-{6-[(3R or S,4S and R OR 3R and S,4S or R)-3-fluoro-4- hydroxypiperidin-1-yl]- [1,3]thiazolo[4,5-b]pyrazin- 2-yl}-5′-methoxy-6-methyl- [4,4′-bipyridine]-3- carboxamide Mixture of cis or trans isomers | 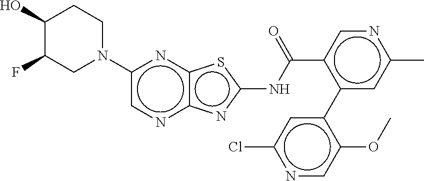 |
| 461 | 2′-chloro-N-(6-((3R or S,4R and S OR 3R and S,4R or S)-3-fluoro-4- hydroxypiperidin-1- yl)thiazolo[4,5-b]pyrazin-2- yl)-5′-methoxy-6-methyl- [4,4′-bipyridine]-3- carboxamide Mixture of trans or cis isomers | 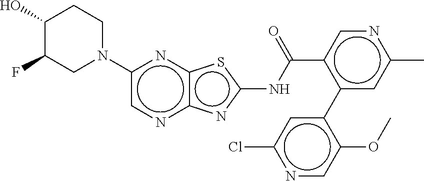 |
| 463 | 2′-chloro-N-(6-((3S or R,4S and R OR 3S and R,4S or R)-4-hydroxy-3- methylpiperidin-1- yl)thiazolo[4,5-b]pyrazin-2- yl)-5′-methoxy-6-methyl- [4,4′-bipyridine]-3- carboxamide Mixture of trans or cis isomers | 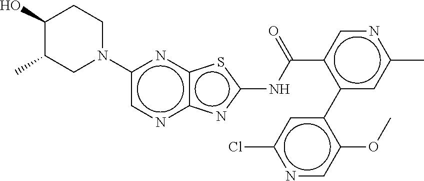 |
| 470 | 2′-chloro-N-(6-((3S or R,4S and R OR 3S and R,4S or R)-4-hydroxy-3- methylpiperidin-1- yl)thiazolo[4,5-b]pyrazin-2- yl)-5′-methoxy-6-methyl- [4,4′-bipyridine]-3- carboxamide Mixture of cis or trans isomers | 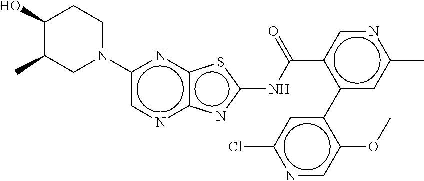 |
| 473 | (R or S)-2′-chloro-5′- methoxy-6-methyl-N-(6- (tetrahydrofuran-3- yl)thiazolo[4,5-b]pyrazin-2- yl)-[4,4′-bipyridine]-3- carboxamide | 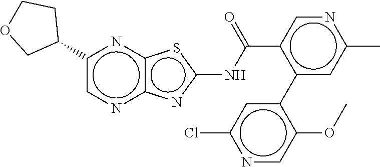 |
| 476 | (cis or trans)-2′-chloro-N- (6-(4- hydroxycyclohexyl)thiazolo [4,5-b]pyrazin-2-yl)-5′- methoxy-6-methyl-[4,4′- bipyridine]-3-carboxamide | 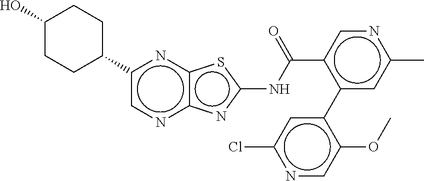 |
| 478 | (trans or cis)-2′-chloro-N- (6-(4- hydroxycyclohexyl)thiazolo [4,5-b]pyrazin-2-yl)-5′- methoxy-6-methyl-[4,4′- bipyridine]-3-carboxamide | 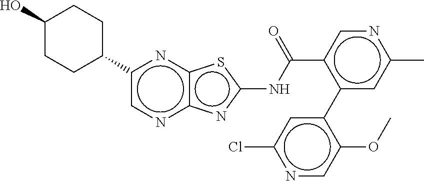 |
| 479 | (S or R)-2′-chloro-5′- methoxy-6-methyl-N-(6- (tetrahydrofuran-3- yl)thiazolo[4,5-b]pyrazin-2- yl)-[4,4′-bipyridine]-3- carboxamide | 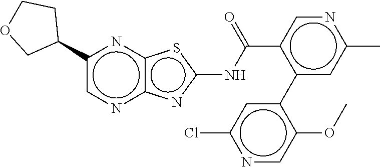 |
| 493 | 6-(3-amino-5- chlorophenoxy)-2′-chloro- N-(6- cyclopropylimidazo[2,1- b][1,3,4]thiadiazol-2-yl)-5′- methoxy-[4,4′-bipyridine]- 3-carboxamide | 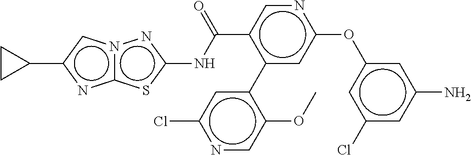 |
| 494 | 6-(3-aminophenoxy)-2′- chloro-N-(6- cyclopropylimidazo[2,1- b][1,3,4]thiadiazol-2-yl)-5′- methoxy-[4,4′-bipyridine]- 3-carboxamide | 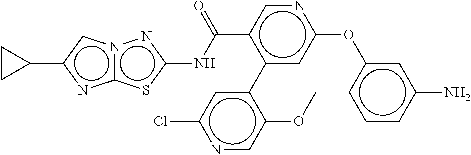 |
| 495 | tert-butyl (3-((2′-chloro-5- ((6-cyclopropylimidazo[2,1- b][1,3,4]thiadiazol-2- yl)carbamoyl)-5′-methoxy- [4,4′-bipyridin]-2- yl)oxy)phenyl)carbamate | 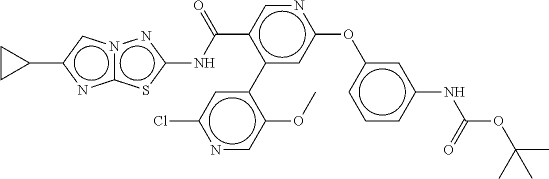 |
| 496 | (cis or trans)-2′-chloro-N- (6-(4- (hydroxymethyl)cyclohexyl) thiazolo[4,5-b]pyrazin-2- yl)-5′-methoxy-6-methyl- [4,4′-bipyridine]-3- carboxamide | 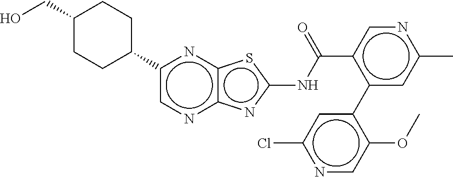 |
| 497 | (trans or cis)-2′-chloro-N- (6-(4- (hydroxymethyl)cyclohexyl) thiazolo[4,5-b]pyrazin-2- yl)-5′-methoxy-6-methyl- [4,4′-bipyridine]-3- carboxamide | 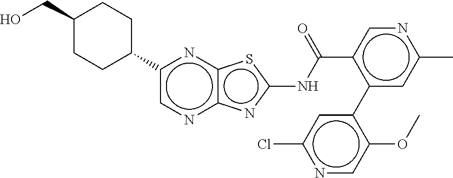 |
| 498 | 2′-chloro-N-(6-((1R or S,3R and S)-3- (hydroxymethyl)cyclopentyl) thiazolo[4,5-b]pyrazin-2- yl)-5′-methoxy-6-methyl- [4,4′-bipyridine]-3- carboxamide | 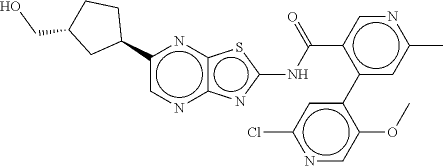 |
| 499 | 2′-chloro-N-(6-((1R,3S)-3- (hydroxymethyl)cyclopentyl) thiazolo[4,5-b]pyrazin-2- yl)-5′-methoxy-6-methyl- [4,4′-bipyridine]-3- carboxamide | 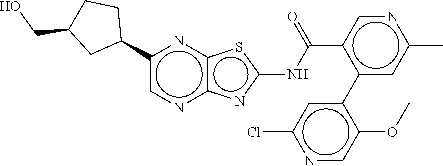 |
| 499A | 2′-chloro-N-(6-((1S,3R)-3- (hydroxymethyl)cyclopentyl) thiazolo[4,5-b]pyrazin-2- yl)-5′-methoxy-6-methyl- [4,4′-bipyridine]-3- carboxamide | 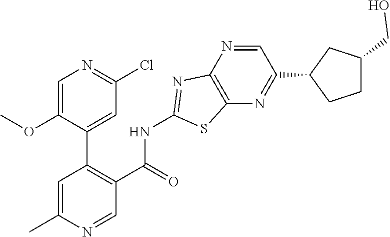 [SMILES: COc1cnc(Cl)cc1-c1cc(C)ncc1C(=O)Nc1nc2ncc([C@H]3CC[C@@H](CO)C3)nc2s1] |
| 511 | 2′-chloro-N-(6-(4-hydroxy- 4- (trifluoromethyl)piperidin- 1-yl)thiazolo[4,5-b]pyrazin- 2-yl)-5′-methoxy-6-methyl- [4,4′-bipyridine]-3- carboxamide | 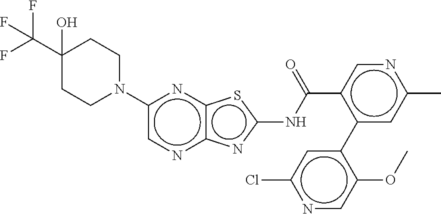 |
| 515 | (R or S)-2′-chloro-5′- methoxy-6-methyl-N-(6- (tetrahydrofuran-3- yl)thiazolo[4,5-b]pyridin-2- yl)-[4,4′-bipyridine]-3- carboxamide | 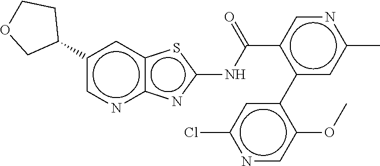 |
| 516 | (S or R)-2′-chloro-5′- methoxy-6-methyl-N-(6- (tetrahydrofuran-3- yl)thiazolo[4,5-b]pyridin-2- yl)-[4,4′-bipyridine]-3- carboxamide | 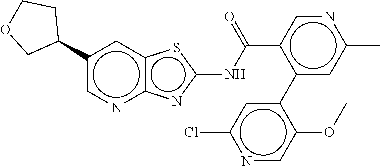 |
| 518 | (cis or trans)-N-(6-(4- Cyanocyclohexyl)thiazolo[4, 5-b]pyrazin-2-yl)-2′- (difluoromethyl)-5′- methoxy-6-methyl-[4,4′- bipyridine]-3-carboxamide | 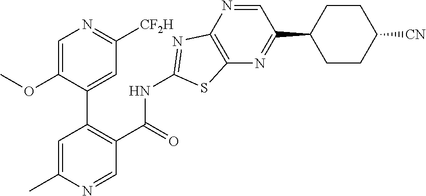 [SMILES: COc1cnc(C(F)F)cc1-c1cc(C)ncc1C(=O)Nc1nc2ncc([C@H]3CC[C@H](C#N)CC3)nc2s1] |
| 519 | (cis or trans)-5-Chloro-N- (6-(4- cyanocyclohexyl)thiazolo[4, 5-b]pyrazin-2-yl)-2- methoxy-6′-methyl-[3,4′- bipyridine]-3′-carboxamide | 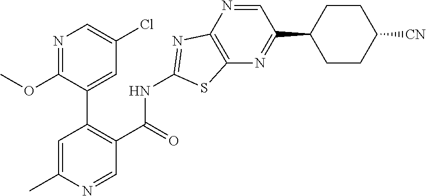 [SMILES: COc1ncc(Cl)cc1-c1cc(C)ncc1C(=O)Nc1nc2ncc([C@H]3CC[C@H](C#N)CC3)nc2s1] |
| 520 | (cis or trans)-N-(6-(4- Cyanocyclohexyl)thiazolo[4, 5-b]pyrazin-2-yl)-4-(6- methoxyimidazo[1,5- a\|pyridin-7-yl)-6- methylnicotinamide | 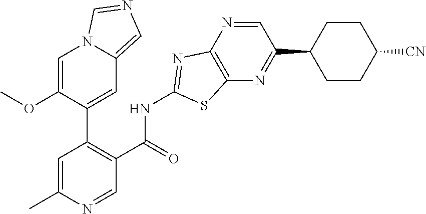 [SMILES: COc1cn2cncc2cc1-c1cc(C)ncc1C(=O)Nc1nc2ncc([C@H]3CC[C@H](C#N)CC3)nc2s1] |
| 521 | (cis or trans)-N-(6-(4- Cyanocyclohexyl)thiazolo[4, 5-b]pyrazin-2-yl)-4-(2- fluoro-6-methoxyphenyl)-6- methylnicotinamide | 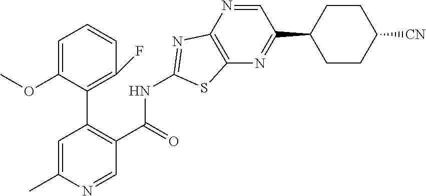 [SMILES: COc1cccc(F)c1-c1cc(C)ncc1C(=O)Nc1nc2ncc([C@H]3CC[C@H](C#N)CC3)nc2s1] |
| 522 | (cis or trans)-N-(6-(4- Cyanocyclohexyl)thiazolo[4, 5-b]pyrazin-2-yl)-4-(6- methoxy-1-methyl-1H- indazol-5-yl)-6- methylnicotinamide | 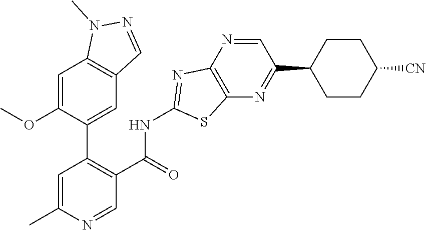 [SMILES: COc1cc2c(cnn2C)cc1-c1cc(C)ncc1C(=O)Nc1nc2ncc([C@H]3CC[C@H](C#N)CC3)nc2s1] |

In some embodiments, the compound is a pharmaceutically acceptable salt of any one of the compounds described in Table 1.

In some embodiments, the compound is a pharmaceutically acceptable salt of any one of the compounds described in Table 2.

In some embodiments, the compound is a pharmaceutically acceptable salt of any one of the compounds described in Table 3.

In some embodiments, the compound is a pharmaceutically acceptable salt of any one of the compounds described in Table 4.

In some embodiments, the compound is a compound described in Tables 1-3, or a prodrug or pharmaceutically acceptable salt thereof.

In some embodiments, the compound is a compound described in Tables 1-3, or a pharmaceutically acceptable salt thereof.

In some embodiments, the compound is a prodrug of a compound described in Tables 1-3, or a pharmaceutically acceptable salt thereof.

In some embodiments, the compound is a compound described in Tables 1-3.

In some embodiments, the compound is a compound described in Tables 1-4, or a prodrug or pharmaceutically acceptable salt thereof.

In some embodiments, the compound is a compound described in Tables 1-4, or a pharmaceutically acceptable salt thereof.

In some embodiments, the compound is a prodrug of a compound described in Tables 1-4, or a pharmaceutically acceptable salt thereof.

In some embodiments, the compound is a compound described in Tables 1-4.

In some embodiments, the compound is not Compound No. 61 (i.e., N-(6-(4-chlorophenyl)thiazolo[4,5-b]pyrazin-2-yl)-4-(5-cyano-2-methoxyphenyl)-6-methylnicotinamide).

In some embodiments, the compound is not Compound No. 75 (i.e., N-(6-(4-chlorophenyl)thiazolo[4,5-b]pyrazin-2-yl)-4-(2-methoxyphenyl)-6-methylnicotinamide).

In some embodiments, the compound is not Compound No. 118 (i.e., N-(5-(4-chlorophenyl)thiazolo[5,4-b]pyridin-2-yl)-4-(2-methoxyphenyl)-6-methylnicotinamide).

In some embodiments, the compound is selected from Compound Nos. 1-60, 62-74, 76-117, and 119-358.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1, or a prodrug or pharmaceutically acceptable salt thereof.

In some embodiments, the compound is an isotopic derivative of any one of the compounds described in Table 1, or a pharmaceutically acceptable salt thereof.

In some embodiments, the compound is an isotopic derivative of any one of prodrugs of the compounds described in Table 1, or a pharmaceutically acceptable salt thereof.

In some embodiments, the compound is an isotopic derivative of any one of the compounds described in Table 1.

In some embodiments, the compound is an isotopic derivative of any one of the compounds described in Table 2, or a prodrug or pharmaceutically acceptable salt thereof.

In some embodiments, the compound is an isotopic derivative of any one of the compounds described in Table 2, or a pharmaceutically acceptable salt thereof.

In some embodiments, the compound is an isotopic derivative of any one of prodrugs of the compounds described in Table 2, or a pharmaceutically acceptable salt thereof.

In some embodiments, the compound is an isotopic derivative of any one of the compounds described in Table 2.

In some embodiments, the compound is an isotopic derivative of any one of the compounds described in Table 3, or a prodrug or pharmaceutically acceptable salt thereof.

In some embodiments, the compound is an isotopic derivative of any one of the compounds described in Table 3, or a pharmaceutically acceptable salt thereof.

In some embodiments, the compound is an isotopic derivative of any one of prodrugs of the compounds described in Table 3, or a pharmaceutically acceptable salt thereof.

In some embodiments, the compound is an isotopic derivative of any one of the compounds described in Table 3.

In some embodiments, the compound is an isotopic derivative of any one of the compounds described in Table 4, or a prodrug or pharmaceutically acceptable salt thereof.

In some embodiments, the compound is an isotopic derivative of any one of the compounds described in Table 4, or a pharmaceutically acceptable salt thereof.

In some embodiments, the compound is an isotopic derivative of any one of prodrugs of the compounds described in Table 4, or a pharmaceutically acceptable salt thereof.

In some embodiments, the compound is an isotopic derivative of any one of the compounds described in Table 4.

In some embodiments, the compound is an isotopic derivative of any one of the compounds described in Tables 1-3, or a prodrug or pharmaceutically acceptable salt thereof.

In some embodiments, the compound is an isotopic derivative of any one of the compounds described in Tables 1-3, or a pharmaceutically acceptable salt thereof.

In some embodiments, the compound is an isotopic derivative of any one of prodrugs of the compounds described in Tables 1-3, or a pharmaceutically acceptable salt thereof.

In some embodiments, the compound is an isotopic derivative of any one of the compounds described in Tables 1-3.

In some embodiments, the compound is an isotopic derivative of any one of the compounds described in Tables 1-4, or a prodrug or pharmaceutically acceptable salt thereof.

In some embodiments, the compound is an isotopic derivative of any one of the compounds described in Tables 1-4, or a pharmaceutically acceptable salt thereof.

In some embodiments, the compound is an isotopic derivative of any one of prodrugs of the compounds described in Tables 1-4, or a pharmaceutically acceptable salt thereof.

In some embodiments, the compound is an isotopic derivative of any one of the compounds described in Tables 1-4.

In some embodiments, the compound is selected from Compound Nos. 1, 10, 24, 25, 59, 207, 211, 212, 224, 235, 238, 239, and 244, or a prodrug or pharmaceutically acceptable salt thereof.

In some embodiments, the compound is selected from Compound Nos. 1, 10, 24, 25, 59, 207, 211, 212, 224, 235, 238, 239, and 244, or a pharmaceutically acceptable salt thereof.

In some embodiments, the compound is selected from Compound Nos. 1, 10, 24, 25, 59, 207, 211, 212, 224, 235, 238, 239, and 244.

In some embodiments, the compound is selected from Compound Nos. 3, 12, 280, 282, 288, 308, 359, 360, 361B, 368, 377, 386, 391, 394, 403, 427, 432, 447, 451A, 454, 462, 464, 467, 486, 489, 498, 502, 504, 506, 514, 518, 520, 521, and 522, or a prodrug or pharmaceutically acceptable salt thereof.

In some embodiments, the compound is selected from Compound Nos. 3, 12, 280, 282, 288, 308, 359, 360, 361B, 368, 377, 386, 391, 394, 403, 427, 432, 447, 451A, 454, 462, 464, 467, 486, 489, 498, 502, 504, 506, 514, 518, 520, 521, and 522, or a pharmaceutically acceptable salt thereof.

In some embodiments, the compound is selected from Compound Nos. 3, 12, 280, 282, 288, 308, 359, 360, 361B, 368, 377, 386, 391, 394, 403, 427, 432, 447, 451A, 454, 462, 464, 467, 486, 489, 498, 502, 504, 506, 514, 518, 520, 521, and 522.

In some embodiments, the compound is selected from Compound Nos. 1, 10, 24, 25, 59, 207, 211, 212, 224, 235, 238, 239, 244, 3, 12, 280, 282, 288, 308, 359, 360, 361B, 368, 377, 386, 391, 394, 403, 427, 432, 447, 451A, 454, 462, 464, 467, 486, 489, 498, 502, 504, 506, 514, 518, 520, 521, and 522, or a prodrug or pharmaceutically acceptable salt thereof.

In some embodiments, the compound is selected from Compound Nos. 1, 10, 24, 25, 59, 207, 211, 212, 224, 235, 238, 239, 244, 3, 12, 280, 282, 288, 308, 359, 360, 361B, 368, 377, 386, 391, 394, 403, 427, 432, 447, 451A, 454, 462, 464, 467, 486, 489, 498, 502, 504, 506, 514, 518, 520, 521, and 522, or a pharmaceutically acceptable salt thereof.

In some embodiments, the compound is selected from Compound Nos. 1, 10, 24, 25, 59, 207, 211, 212, 224, 235, 238, 239, 244, 3, 12, 280, 282, 288, 308, 359, 360, 361B, 368, 377, 386, 391, 394, 403, 427, 432, 447, 451A, 454, 462, 464, 467, 486, 489, 498, 502, 504, 506, 514, 518, 520, 521, and 522.

In some embodiments, the compound is a compound obtainable by, or obtained by, a method described herein; optionally, the method comprises one or more steps described in Schemes 1-11.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (I) is isotopically enriched with regard to, or labelled with, one or more isotopes as compared to the corresponding compound of Formula (I). In some embodiments, the isotopic derivative is enriched with regard to, or labelled with, one or more atoms selected from ²H, ¹³C, ¹⁴C, ¹⁵N, ¹⁸O, ²⁹Si, ³¹P, and 34S. In some embodiments, the isotopic derivative is a deuterium labeled compound (i.e., being enriched with ²H with regard to one or more atoms thereof). In some embodiments, the compound is a ¹⁸F labeled compound.

In some embodiments, the compound is a ¹²³I labeled compound, a ¹²⁴I labeled compound, a ¹²⁵I labeled compound, a ¹²⁹I labeled compound, a ¹³¹I labeled compound, a ¹³⁵I labeled compound, or any combination thereof. In some embodiments, the compound is a 33S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵I, ¹²⁹I, ¹³¹I, ¹³⁵I, ³²S, ³⁴S, ³⁵S, and/or ³⁶S labeled compound, can be prepared using any of a variety of art-recognized techniques. For example, the deuterium labeled compound can generally be prepared by carrying out the procedures disclosed in the Schemes and/or in the Examples described herein, by substituting a ¹⁸F, ¹²³I ¹²⁴I, ¹²⁵I, ¹²⁹I, ¹³¹I, ¹³⁵I, ³S, ³⁴S ³⁵S, and/or ³⁶S labeled reagent for a non-isotope labeled reagent.

A compound of the invention or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³²S, ³⁴S ³⁵S, and ³⁶S atom(s) is within the scope of the invention. Further, substitution with isotope (e.g., ¹⁸F, ¹²³I, ¹²⁴I, ¹²⁵I, ¹²⁹I, ¹³¹I, ¹³⁵I, ³S 34S ³⁵S, and/or ³⁶S) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

The various functional groups and substituents making up the compounds of the Formula (I) are typically chosen such that the molecular weight of the compound does not exceed 1000 daltons. More usually, the molecular weight of the compound will be less than 900, for example less than 800, or less than 750, or less than 700, or less than 650 daltons. More conveniently, the molecular weight is less than 600 and, for example, is 550 daltons or less.

A suitable pharmaceutically acceptable salt of a compound of the disclosure is, for example, an acid-addition salt of a compound of the disclosure which is sufficiently basic, for example, an acid-addition salt with, for example, an inorganic organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

It will be understood that while compounds disclosed herein may be presented in one particular configuration. Such particular configuration is not to be construed as limiting the disclosure to one or another isomer, tautomer, regioisomer or stereoisomer, nor does it exclude mixtures of isomers, tautomers, regioisomers or stereoisomers. In some embodiments, the presentation of a compound herein in a particular configuration intends to encompass, and to refer to, each of the available isomers, tautomers, regioisomers, and stereoisomers of the compound, or any mixture thereof; while the presentation further intends to refer to the specific configuration of the compound.

It will be understood that while compounds disclosed herein may be presented without specified configuration (e.g., without specified stereochemistry). Such presentation intends to encompass all available isomers, tautomers, regioisomers, and stereoisomers of the compound.

In some embodiments, the presentation of a compound herein without specified configuration intends to refer to each of the available isomers, tautomers, regioisomers, and stereoisomers of the compound, or any mixture thereof.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R and S-sequencing rules of Cahn and Prelog, or by the manner in which—the molecule rotates the plane of polarized light and designated as dextrorotatory or levorotatory (i.e., as (+) or (−−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centers; such compounds can therefore be produced as individual (R) or (S) stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centers (E and Z isomers). It is to be understood that the present disclosure encompasses all optical, diastereoisomers and geometric isomers and mixtures thereof that possess polymerase Θ inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess polymerase Θ inhibitory activity.

It is also to be understood that certain compounds of any one of the Formulae disclosed herein may exhibit polymorphism, and that the disclosure encompasses all such forms, or mixtures thereof, which possess polymerase Θ inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of Formula (I) include all such forms. For the avoidance of doubt, where a compound can exist in one of several tautomeric forms, and only one is specifically described or shown, all others are nevertheless embraced by Formula (I). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

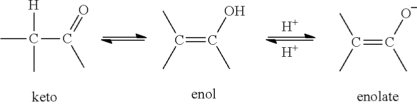

Compounds of any one of the Formulae disclosed herein containing an amine function may also form N-oxides. A reference herein to a compound of Formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidizing agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂ carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁-4 alkylamine such as methylamine, a (C₁-C₄ alkyl)₂ amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Methods of Synthesis

In some aspects, the present disclosure provides a method of preparing a compound of the present disclosure.

In some aspects, the present disclosure provides a method of a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of Formula (I) has been synthesized by any one of the processes defined herein, the processes may then further comprise the additional steps of (i) removing any protecting groups present; (ii) converting the compound Formula (I) into another compound of Formula (I); (iii) forming a pharmaceutically acceptable salt, hydrate or solvate thereof; and/or (iv) forming a prodrug thereof.

The resultant compounds of Formula (I) can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions.

Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognize which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

General routes for the preparation of a compound of the application are described in Schemes 1-11 herein.

While the present invention has been described in conjunction with the specific examples set forth below, many alternatives, modifications, and variations thereof will be apparent to those of ordinary skill in the art. In some cases, the order of carrying out the steps of the reaction schemes may be varied to facilitate the reaction or to avoid unwanted reaction products.

Starting materials and intermediates are purchased from commercial sources, made from known procedures, or are otherwise illustrated.

Several methods for preparing the compounds of this invention are described in the following Schemes and Examples. Unless otherwise indicated, all variables are as previously defined.

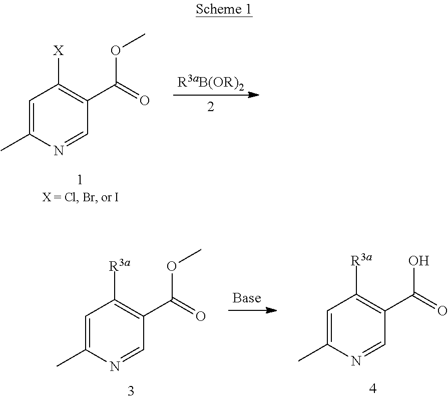

In Scheme 1, Suzuki coupling of boronic ester or boronic acid 2 with halopyridine 1 affords ester 3. The ester can be hydrolyzed under basic conditions (e.g., with the use of NaOH) to afford carboxylic acid 4.

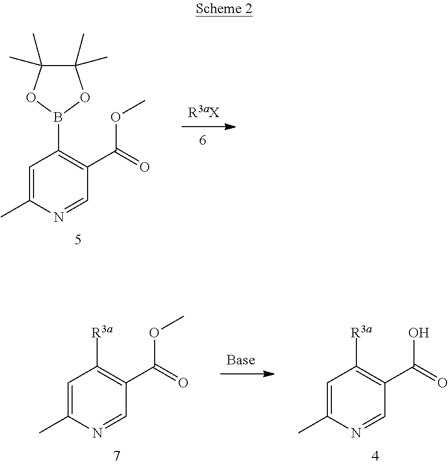

In Scheme 2, carboxylic acid 4 can be made in two steps via Suzuki coupling with boronate 5 and aryl halide 6. Ester 7 can be hydrolyzed under basic conditions (e.g., with the use of NaOH) to form carboxylic acid 4.

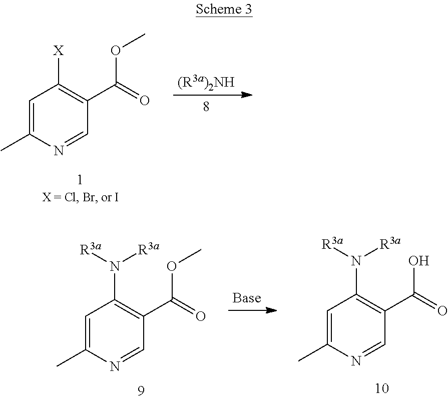

In Scheme 3, S_{N}Ar with amine 8 and halopyridine 1 affords ester 9. Basic hydrolysis (e.g., with the use of NaOH) affords carboxylic acid 10.

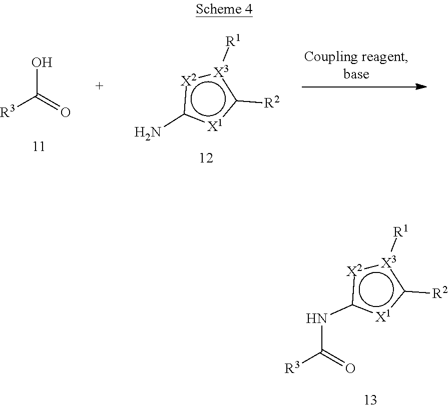

In Scheme 4, amide 13 can be made in one step from carboxylic acid 11 and amine 12 via an amide coupling using a coupling reagent (e.g., through the use of HATU or other common amide coupling reagents) and base (e.g., DIPEA).

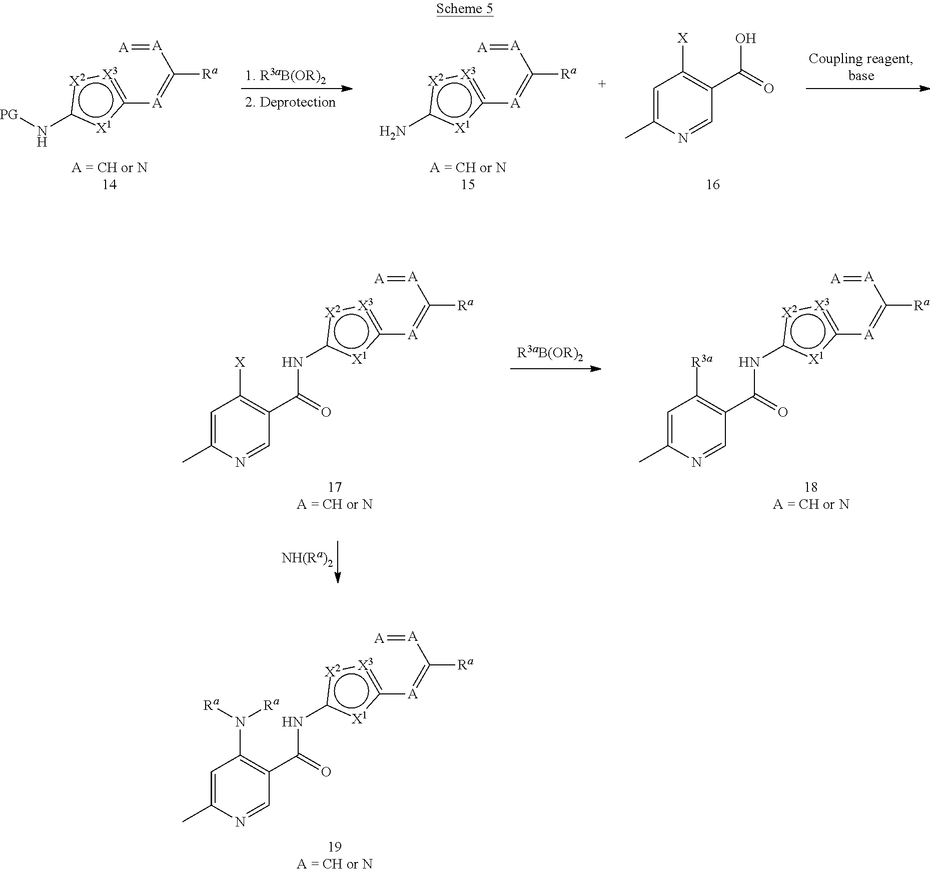

In Scheme 5, amine 15 can be formed via Suzuki coupling with aryl halide 14 and boronic esters or boronic acids, followed by a deprotection step (e.g., a Boc group would be removed through the use of an acid). Amide coupling (e.g., through the use of HATU or other common amide coupling reagents) with carboxylic acid 16 affords halopyridine 17, which can be functionalized via a Suzuki reaction to afford amide 18. Alternatively, amines can be added to 17 via S_{N}Ar to afford 19.

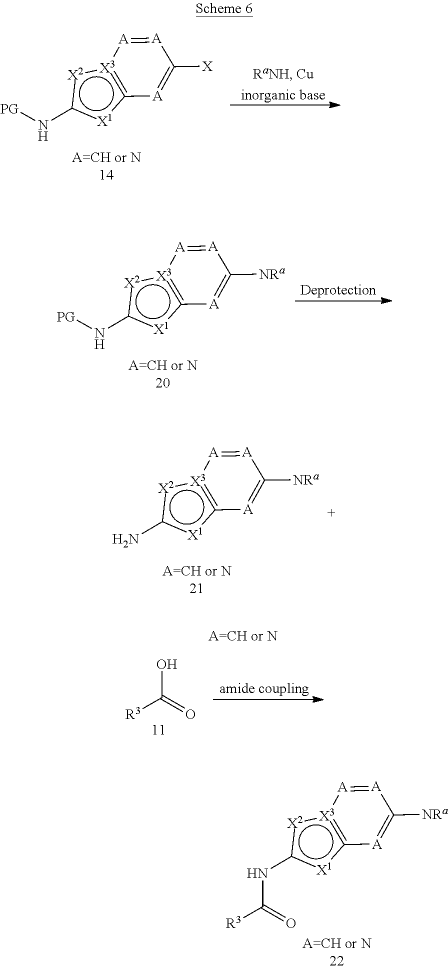

In Scheme 6, amine 20 can be made via Ullman coupling of aryl halide 14 and an amine, followed by a deprotection step to afford amine 21 (e.g., a Boc group would be removed through the use of an acid). Amine 21 can be converted to amide 22 via amide coupling (e.g., through the use of HATU or other common amide coupling reagents) with carboxylic acid 11.

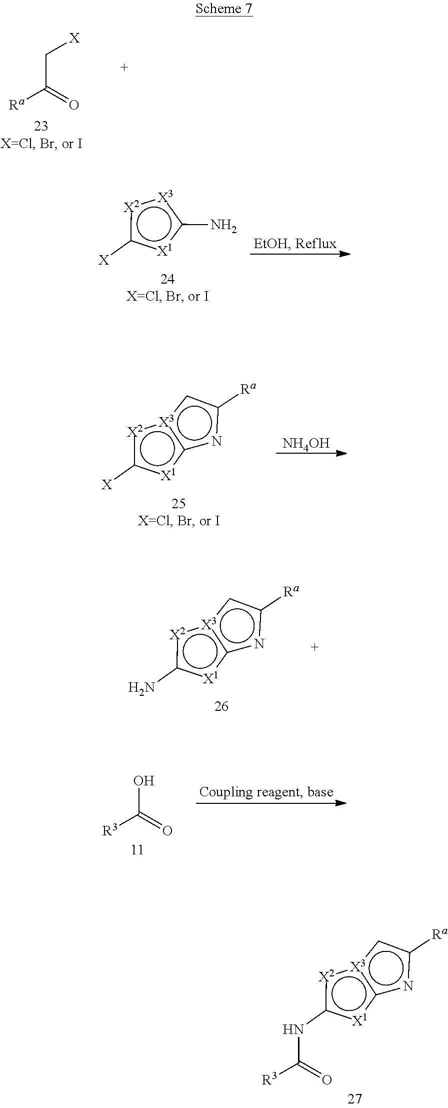

In Scheme 7, aryl halide 25 can be formed via addition of amine 24 to alpha-halo ketone 23. Amination affords primary amine 26, and amide coupling (e.g., through the use of EDCI or other common coupling reagents) with carboxylic acid 11 affords amide 27.

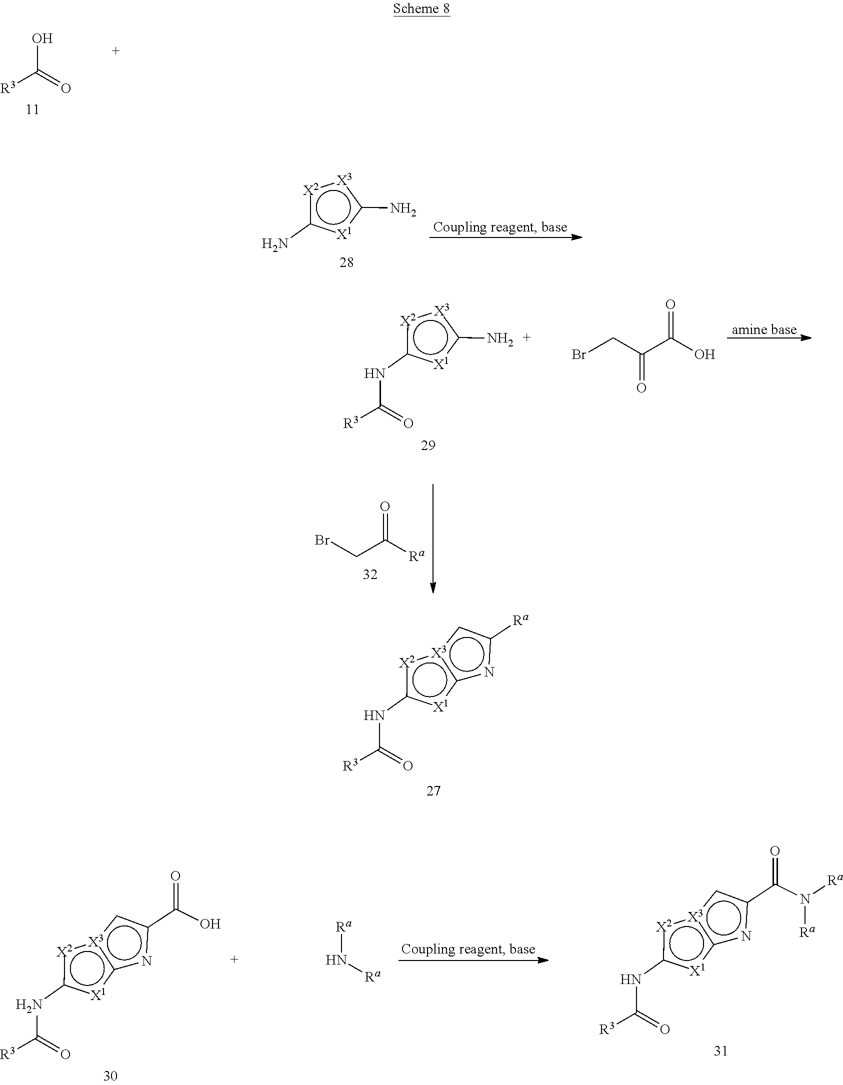

In Scheme 8, amide coupling between acid 11 and diamine 28 affords amide 29. Condensation reaction between amine 29 and alpha-halo ketone affords carboxylic acid 30, which can undergo amide coupling with amines to afford amide 31. Alternatively, amine 29 can be condensed with alpha-halo ketones 32 to afford amide 27.

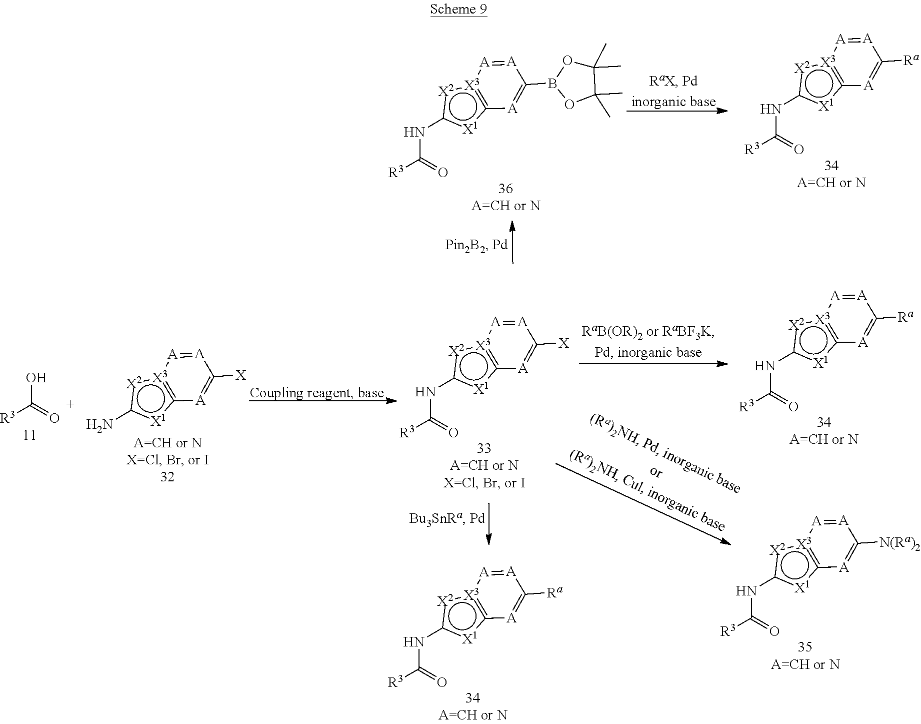

In Scheme 9, amide 33 can be formed via amide coupling between carboxylic acid 11 and amine 32. Suzuki reaction with aryl halide 33 affords amide 34. Alternatively, Stille coupling with tin reagents and aryl bromide 33 affords amide 34. Aryl bromide 33 can be converted to aryl amine 35 via Buchwald or Ullman coupling. In another approach, aryl halide 33 can be converted to boronate 36 followed by Suzuki coupling with aryl halides to afford amide 34.

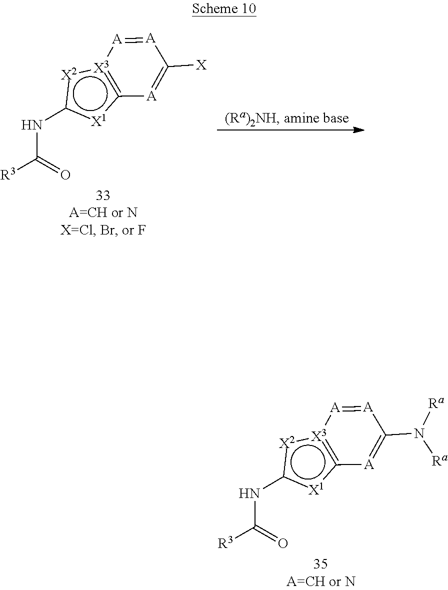

In Scheme 10, aryl bromide 33 can be converted to aryl amine 35 via S_{N}Ar.

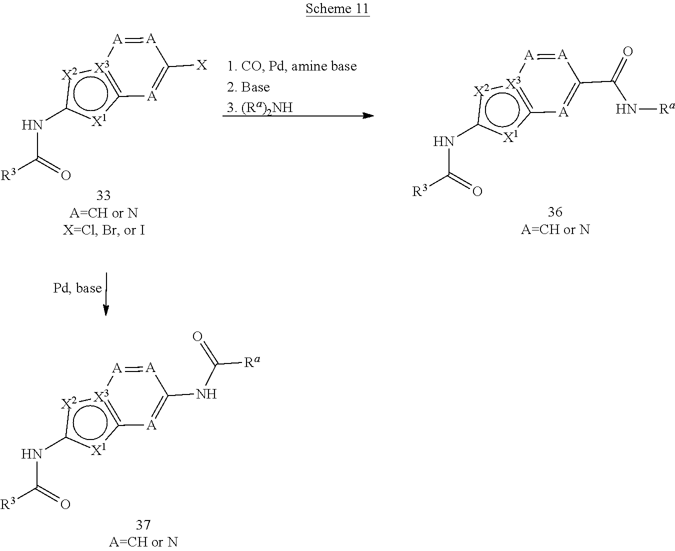

In Scheme 11, aryl halide 33 can be converted to amide 36 via a three step process, including carbonylation, hydrolysis under basic conditions, and amide coupling. Alternatively, aryl bromide 33 can be converted to amide 37 via palladium-catalyzed cross coupling of a primary amide.

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the biological assay is described in the Examples herein.

In some embodiments, the biological assay is an ATPase assay.

In some embodiments, the ATPase assay a compound of the present disclosure is combined with biotinylated Avi-POLΘ[2-894], a 50 nucleotide poly-thymine repeat single-stranded DNA, and ATP in buffer. In some embodiments, the mixture is incubated and ADP-Glo Reagent added, followed by a subsequent incubation. In some embodiments, a Kinase Detection Reagent may be added, followed by a third incubation.

In some embodiments, the activity is measured via luminescence signal (e.g., measured with the EnVision multimode plate reader).

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising a compound described in Tables 1-4. In some embodiments, the present disclosure provides a pharmaceutical composition comprising at least one compound selected from Tables 1-4. In some aspects, the present disclosure provides a pharmaceutical composition comprising the compound of any one of the preceding claims or a pharmaceutically acceptable salt thereof, and a pharmaceutically acceptable diluent or carrier.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions.

The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

An effective amount of a compound of the present disclosure for use in therapy is an amount sufficient to treat or prevent a polymerase Θ related condition referred to herein, slow its progression and/or reduce the symptoms associated with the condition.

An effective amount of a compound of the present disclosure for use in therapy is an amount sufficient to treat a polymerase Θ related condition referred to herein, slow its progression and/or reduce the symptoms associated with the condition.

The size of the dose for therapeutic or prophylactic purposes of a compound of Formula (I) will naturally vary according to the nature and severity of the conditions, the age and sex of the animal or patient and the route of administration, according to well-known principles of medicine.

### Methods of Use

In some aspects, the present disclosure provides a method of modulating DNA polymerase Θ activity, comprising contacting a cell with a compound of the present disclosure or a pharmaceutically acceptable salt thereof.

In some aspects, the present disclosure provides a method of modulating DNA polymerase Θ activity (e.g., in vitro or in vivo), comprising contacting a cell with an effective amount of a compound of the present disclosure or a pharmaceutically acceptable salt thereof.

In some aspects, the present disclosure provides a method of modulating DNA polymerase Θ activity (e.g., in vitro or in vivo), comprising contacting a cell with a compound of the present disclosure or a pharmaceutically acceptable salt thereof.

In some aspects, the present disclosure provides a method of modulating DNA polymerase Θ activity (e.g., in vitro or in vivo), comprising contacting a cell with an effective amount of a compound of the present disclosure or a pharmaceutically acceptable salt thereof.

In some aspects, the present disclosure provides a method of modulating DNA polymerase Θ activity (e.g., in vitro or in vivo), comprising contacting a cell with a compound of the present disclosure or a pharmaceutically acceptable salt thereof.

In some aspects, the present disclosure provides a method of treating or preventing a disease or disorder in a subject in need thereof, comprising administering to the subject a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a disease or disorder disclosed herein in a subject in need thereof, comprising administering to the subject a compound of the present disclosure or a pharmaceutically acceptable salt thereof.

In some aspects, the present disclosure provides a method of treating a disease or disorder disclosed herein in a subject in need thereof, comprising administering to the subject a compound of the present disclosure or a pharmaceutically acceptable salt thereof, or a pharmaceutical composition of the present disclosure.

In some aspects, the present disclosure provides a method for inhibiting DNA repair by DNA polymerase Θ in a cancer cell comprising contacting the cell with an effective amount of a compound of the present disclosure or a pharmaceutically acceptable salt thereof, or a pharmaceutical composition of the present disclosure. In some embodiments, the cancer is HR deficient cancer.

In some aspects, the present disclosure provides a method for treating and/or preventing a cancer in a patient, wherein the cancer is characterized by a reduction or absence of BRCA gene expression, the absence of the BRCA gene, or reduced function of BRCA protein, comprising administering to the subject a compound of the present disclosure or a pharmaceutically acceptable salt thereof, or a pharmaceutical composition of the present disclosure.

In some embodiments, the disease or disorder is associated with an implicated DNA polymerase Θ activity. In some embodiments, the disease or disorder is a disease or disorder in which DNA polymerase Θ activity is implicated.

In some embodiments, the disease or disorder is associated with an implicated DNA polymerase Θ activity. In some embodiments, the disease or disorder is a disease or disorder in which DNA polymerase Θ activity is implicated.

In some embodiments, the disease or disorder is cancer.

In some aspects, the present disclosure provides a method of treating or preventing cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing cancer in a subject in need thereof, comprising administering to the subject a compound of the present disclosure or a pharmaceutically acceptable salt thereof, or a pharmaceutical composition of the present disclosure.

In some aspects, the present disclosure provides a method of treating cancer in a subject in need thereof, comprising administering to the subject a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof, or a pharmaceutical composition of the present disclosure for use in modulating DNA polymerase Θ activity.

In some aspects, the present disclosure provides a compound of the present disclosure or a pharmaceutically acceptable salt thereof for use in modulating DNA polymerase Θ activity (e.g., in vitro or in vivo).

In some aspects, the present disclosure provides a compound of the present disclosure or a pharmaceutically acceptable salt thereof for use in modulating DNA polymerase Θ activity (e.g., in vitro or in vivo).

In some aspects, the present disclosure provides a compound of the present disclosure or a pharmaceutically acceptable salt thereof for inhibiting DNA repair by DNA polymerase Θ in a cell. In some embodiments, the cell is HR deficient cell.

In some aspects, the present disclosure provides a compound of the present disclosure or a pharmaceutically acceptable salt thereof for use in the treatment and/or prevention of a disease in a patient, wherein the disease is characterized by overexpression of DNA polymerase Θ.

In some aspects, the present disclosure provides a compound of the present disclosure or a pharmaceutically acceptable salt thereof for use in the treatment and/or prevention of a cancer in a patient, wherein the cancer is characterized by a reduction or absence of BRCA gene expression, the absence of the BRCA gene, or reduced function of BRCA protein.

In some embodiments, BRCA is BRCA1. In some embodiments, BRCA is BRCA2.

In some aspects, the present disclosure provides a compound of the present disclosure or a pharmaceutically acceptable salt thereof for use in the treatment and/or prevention of a HR deficient cancer in a patient.

In some aspects, the present disclosure provides a compound of the present disclosure or a pharmaceutically acceptable salt thereof, or a pharmaceutical composition of the present disclosure for use in treating or preventing a disease or disorder.

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing cancer in a subject in need thereof.

In some aspects, the present disclosure provides a compound of the present disclosure or a pharmaceutically acceptable salt thereof for use in treating cancer in a subject in need thereof.

In some aspects, the present disclosure provides use of a compound of the present disclosure or a pharmaceutically acceptable salt thereof in the manufacture of a medicament for modulating DNA polymerase Θ activity.

In some aspects, the present disclosure provides use of a compound of the present disclosure or a pharmaceutically acceptable salt thereof in the manufacture of a medicament for modulating DNA polymerase Θ activity (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing cancer in a subject in need thereof.

In some aspects, the present disclosure provides use of a compound of the present disclosure or a pharmaceutically acceptable salt thereof in the manufacture of a medicament for treating cancer in a subject in need thereof.

The present disclosure provides compounds that function as modulators of DNA polymerase Θ activity.

In some embodiments, the compounds of the present disclosure are antagonists of the DNA polymerase Θ receptor.

In some embodiments, the modulation of the DNA polymerase Θ receptor is activation of the DNA polymerase Θ receptor.

In some embodiments, modulation is inhibition.

Effectiveness of compounds of the disclosure can be determined by industry-accepted assays/disease models according to standard practices of elucidating the same as described in the art and are found in the current general knowledge.

The present disclosure also provides a method of treating a disease or disorder in which DNA polymerase Θ activity is implicated in a patient in need of such treatment, said method comprising administering to said patient a therapeutically effective amount of a compound, or a pharmaceutically acceptable salt thereof, or a pharmaceutical composition as defined herein.

In some embodiments, the cancer is lymphoma, soft tissue, rhabdoid, multiple myeloma, gastric, peripheral nervous system, rhabdomyosarcoma, bone, colorectal, mesothelioma, prostate, breast, ovarian, uterine, lung, fibroblast, central nervous system, urinary tract, upper aerodigestive, leukemia, kidney, skin, esophagus, and pancreas (data from large scale drop out screens in cancer cell lines indicate that some cell lines from the above cancers are dependent on polymerase Θ for proliferation see https://depmap.org/portal/).

In some embodiments, a HR-deficient cancer is breast cancer. Breast cancer includes, but is not limited to, lobular carcinoma in situ, a ductal carcinoma in situ, an invasive ductal carcinoma, triple negative, HER positive, estrogen receptor positive, progesterone receptor positive, HER and estrogen receptor positive, HER and estrogen and progesterone receptor, positive inflammatory breast cancer, Paget disease of nipple, Phyllodes tumor, angiosarcoma, adenoid cystic carcinoma, low-grade adenosquamous carcinoma, medullary carcinoma, mucinous carcinoma, papillary carcinoma, tubular carcinoma, metaplastic carcinoma, micropapillary carcinoma, and mixed carcinoma. In a second embodiment, HR-deficient cancer is ovarian cancer. Ovarian cancer includes, but is not limited to, epithelial ovarian carcinomas, maturing teratomas, dysgerminomas, endodermal sinus tumors, granulosa-theca tumors, Sertoli-Leydig cell tumors, and primary peritoneal carcinoma. In some cases, ovarian cancer originated from cells of the fallopian tube.

In some embodiments, the cancer is selected from ovarian, prostate, breast, pancreatic or uterine cancer.

In some embodiments, the cancer is resistant to treatment with at least one PARP inhibitor.

In some embodiments, a compound of the present disclosure is administered after treatment with at least one PARP inhibitor.

In some embodiments, the cancer is resistant to treatment with one PARP inhibitor.

In some embodiments, the subject received prior treatment with PARP inhibitor and the cancer is relapsed or refractory.

In some embodiments, the PARP inhibitor is a selective PARP inhibitor.

In some embodiments, the PARP inhibitor is a non-selective PARP inhibitor.

In some embodiments, the PARP inhibitor is olaparib.

In some embodiments, the cancer has a deficiency in a DNA damage repair process.

In some embodiments, the cancer is sensitive to POLO inhibition.

In some embodiments, the cancer has evidence of elevated POLO activity.

In some embodiments, the cancer has elevated expression of POLO mRNA or protein.

In some embodiments, the cancer has elevated expression of POLO mRNA.

In some embodiments, the cancer has elevated expression of POLO protein.

In some embodiments, the cancer is classified by a genotype.

In some embodiments, the genotype has a modulated function.

In some embodiments, the modulated function is an inactivating mutation, deletion, or other genomic alteration.

In some embodiments, the genotype is loss of mRNA or protein expression.

In some embodiments, the cancer has modulated function of at least one gene.

In some embodiments, the gene is selected from ATM, BARD1, BRIP1, CDK12, CHEK1, CHEK2, FANCL, PALB2, RAD51B, RAD51C, RAD51D, RAD54L.

In some embodiments, the cancer is a homologous recombination deficient (HRD) cancer.

In some embodiments, the cancer is classified as an HRD cancer because the tumor is unable to accurately repair double-strand breaks in DNA via homologous recombination.

In some embodiments, the mutation is in a gene that, when lost, causes HRD.

In some embodiments, the cancer has a compromised homologous recombination (HR) or non-homologous DNA end joining (NHEJ) repair pathway.

In some embodiments, the cancer with a compromised HR is dependent on PolΘ activity.

In some embodiments, the cancer with a compromised NHEJ is dependent on PolΘ activity.

In some embodiments, the cancer is a tumor. In some embodiments, the cancer is a solid tumor.

### Routes of Administration

Compounds of the present disclosure, or pharmaceutically acceptable salts thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e., by itself the adjuvant may only have minimal therapeutic benefit, but in combination with another therapeutic agent, the overall therapeutic benefit to the individual is enhanced). Alternatively, by way of example only, the benefit experienced by an individual may be increased by administering the compound of Formula (I) with another therapeutic agent (which also includes a therapeutic regimen) that also has therapeutic benefit.

In the instances where the compound of the present disclosure is administered in combination with other therapeutic agents, the compound of the disclosure need not be administered via the same route as other therapeutic agents, and may, because of different physical and chemical characteristics, be administered by a different route. For example, the compound of the disclosure may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease in which DNA polymerase Θ activity is implicated comprising a compound of the disclosure as defined hereinbefore, or a pharmaceutically acceptable salt thereof, and another suitable agent.

According to a further aspect of the disclosure there is provided a pharmaceutical composition which comprises a compound of the disclosure, or a pharmaceutically acceptable salt thereof, in combination with a suitable, in association with a pharmaceutically acceptable diluent or carrier.

In addition to its use in therapeutic medicine, compounds of Formula (I) and pharmaceutically acceptable salts thereof are also useful as pharmacological tools in the development and standardization of in vitro and in vivo test systems for the evaluation of the effects of modulators of DNA polymerase Θ activity in laboratory animals such as dogs, rabbits, monkeys, mini-pigs, rats and mice, as part of the search for new therapeutic agents.

In any of the above-mentioned pharmaceutical composition, process, method, use, medicament, and manufacturing features of the instant disclosure, any of the alternate embodiments of macromolecules of the present disclosure described herein also apply.

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray or powder); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrastemal; by implant of a depot or reservoir, for example, subcutaneously or intramuscularly.

### EXEMPLARY EMBODIMENTS

Exemplary Embodiment No. 1. A compound of Formula (I):

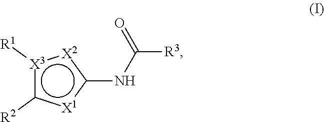

or a pharmaceutically acceptable salt, solvate, clathrate, hydrate, stereoisomer, or tautomer thereof, wherein:
X¹ is CH, S, or N;
X² is N, S, or O;
X³ is C or N;
R¹ and R², together with the atoms to which they are attached, form a C₆-C₁₀ aryl or 5- to 10-membered heteroaryl, wherein the C₆-C₁₀ aryl or 5- to 10-membered heteroaryl is optionally substituted with one or more R^{a};
each R^{a} independently is oxo, halo, cyano, —OH, —NH₂, —C(O)N(R^{b})(R^{c}), —N(R^{b})C(O)(R^{c}), C₁-C₆ alkyl, C₂-C₆ alkenyl, C₂-C₆ alkynyl, C₁-C₆ haloalkyl, C₃-C₁₀ cycloalkyl, C₄-C₁₀ cycloalkenyl, C₆-C₁₀ aryl, 5- to 10-membered heteroaryl, 3- to 10-membered heterocycloalkyl, or 4- to 10-membered heterocycloalkenyl, wherein the C₁-C₆ alkyl, C₂-C₆ alkenyl, C₂-C₆ alkynyl, C₁-C₆ haloalkyl, C₃-C₁₀ cycloalkyl, C₄-C₁₀ cycloalkenyl, C₆-C₁₀ aryl, 5- to 10-membered heteroaryl, 3- to 10-membered heterocycloalkyl, or 4- to 10-membered heterocycloalkenyl is optionally substituted with one or more R^{a1};
each R^{b} and R^{c} independently is H, C₁-C₆ alkyl, C₃-C₁₀ cycloalkyl, or 3- to 10-membered heterocycloalkyl, wherein the C₁-C₆ alkyl, C₃-C₁₀ cycloalkyl, or 3- to 10-membered heterocycloalkyl is optionally substituted with one or more —OH or C₁-C₆ haloalkyl; or
   R^{b} and R^{c}, together with the atoms to which they are attached, form a 3- to 10-membered heterocycloalkyl, wherein the 3- to 10-membered heterocycloalkyl is optionally substituted with one or more —OH, —O(C₁-C₆ haloalkyl), —O(C₁-C₆ alkyl), —C(O)(O—(C₁-C₆ alkyl)), C₁-C₆ alkyl, or —N(C₁-C₆ alkyl)₂;
each R^{a1} independently is oxo, halo, cyano, —OH, —O(C₃-C₁₀ cycloalkyl), —NH₂, —NH(C₁-C₆ alkyl), —N(C₁-C₆ alkyl)₂, —C(O)OH, —C(O)O(C₁-C₆ alkyl), C₁-C₆ alkyl, C₂-C₆ alkenyl, C₂-C₆ alkynyl, C₁-C₆ haloalkyl, C₁-C₆ alkoxy, C₁-C₆ haloalkoxy, —O—(C₃-C₁₀ cycloalkyl), C₃-C₁₀ cycloalkyl, 3- to 10-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl is optionally substituted with one or more R^{a2};
each R^{a2} independently is oxo, halo, cyano, —OH, or —NH₂; R³ is C₆-C₁₀ aryl or 6- to 10-membered heteroaryl, wherein the C₆-C₁₀ aryl or 6- to 10-membered heteroaryl is substituted with one or more R^{3a};
each R^{3a} independently is halo, cyano, —OH, —NH₂, C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, —O(C₆-C₁₀ aryl), C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, —O(C₆-C₁₀ aryl), C₆-C₁₀ aryl, or 5- to 10-membered heteroaryl is optionally substituted with one or more R^{3a1}; and
each R^{3a1} independently is oxo, halo, cyano, —NH₂, —NH—C(O)O(C₁-C₆ alkyl), —C(O)(C₁-C₆ alkyl), —C(O)NH₂, —C(O)(O—(C₁-C₆ alkyl)), C₁-C₆ alkyl optionally substituted with one or more C₃-C₁₀ cycloalkyl, C₂-C₆ alkenyl, C₂-C₆ alkynyl, C₁-C₆ haloalkyl, C₁-C₆ alkoxy, —O(C₁-C₆ haloalkyl), C₃-C₁₀ cycloalkyl, C₆-C₁₀ aryl, or 5- to 10-membered heteroaryl; wherein, when R^{3a} is halo and R¹ and R², together with the atoms to which they are attached, form a 6-membered heteroaryl then R^{a} is not —CF₃.

Exemplary Embodiment No. 2. A compound of Formula (I):

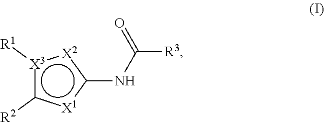

or a pharmaceutically acceptable salt, solvate, clathrate, hydrate, stereoisomer, or tautomer thereof, wherein:
X¹ is CH, S, or N;
X² is N, S, or O;
X³ is C or N;
R¹ and R², together with the atoms to which they are attached, form a C₆-C₁₀ aryl or 5- to 10-membered heteroaryl, wherein the C₆-C₁₀ aryl or 5- to 10-membered heteroaryl is optionally substituted with one or more R^{a};
each R^{a} independently is oxo, halo, cyano, —OH, —NH₂, —C(O)N(R^{b})(R^{c}), —N(R^{b})C(O)(R^{c}), C₁-C₆ alkyl, C₂-C₆ alkenyl, C₂-C₆ alkynyl, C₁-C₆ haloalkyl, C₃-C₁₀ cycloalkyl, C₆-C₁₀ aryl, 5- to 10-membered heteroaryl, or 3- to 10-membered heterocycloalkyl, wherein the C₁-C₆ alkyl, C₂-C₆ alkenyl, C₂-C₆ alkynyl, C₁-C₆ haloalkyl, C₃-C₁₀ cycloalkyl, C₆-C₁₀ aryl, 5- to 10-membered heteroaryl, or 3- to 10-membered heterocycloalkyl is optionally substituted with one or more R^{a1};
each R^{b} and R^{c} independently is H, C₁-C₆ alkyl, C₃-C₁₀ cycloalkyl, or 3- to 10-membered heterocycloalkyl, wherein the C₁-C₆ alkyl, C₃-C₁₀ cycloalkyl, or 3- to 10-membered heterocycloalkyl is optionally substituted with one or more —OH or C₁-C₆ haloalkyl; or
R^{b} and R^{c}, together with the atoms to which they are attached, form a 3- to 10-membered heterocycloalkyl, wherein the 3- to 10-membered heterocycloalkyl is optionally substituted with one or more —OH, —O(C₁-C₆ haloalkyl), —O(C₁-C₆ alkyl), —C(O)(O—(C₁-C₆ alkyl)), C₁-C₆ alkyl, or —N(C₁-C₆ alkyl)₂;
each R^{a1} independently is oxo, halo, cyano, —OH, —O(C₃-C₁₀ cycloalkyl), —NH₂, —NH(C₁-C₆ alkyl), —N(C₁-C₆ alkyl)₂, —C(O)OH, —C(O)O(C₁-C₆ alkyl), C₁-C₆ alkyl, C₂-C₆ alkenyl, C₂-C₆ alkynyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl is optionally substituted with one or more R^{a2};
each R^{a2} independently is oxo, halo, cyano, —OH, or —NH₂; R³ is C₆-C₁₀ aryl or 6- to 10-membered heteroaryl, wherein the C₆-C₁₀ aryl or 6- to 10-membered heteroaryl is substituted with one or more R^{3a}, each R^{3a} independently is halo, cyano, —OH, —NH₂, C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, C₆-C₁₀ aryl, or 5- to 10-membered heteroaryl is optionally substituted with one or more R^{3a1}; and
each R^{3a1} independently is oxo, halo, cyano, —C(O)(C₁-C₆ alkyl), —C(O)NH₂, —C(O)(O—(C₁-C₆ alkyl)), C₁-C₆ alkyl optionally substituted with one or more C₃-C₁₀ cycloalkyl, C₂-C₆ alkenyl, C₂-C₆ alkynyl, C₁-C₆ haloalkyl, C₁-C₆ alkoxy, —O(C₁-C₆ haloalkyl), C₃-C₁₀ cycloalkyl, C₆-C₁₀ aryl, or 5- to 10-membered heteroaryl;
wherein, when R^{3a} is halo and R¹ and R², together with the atoms to which they are attached, form a 6-membered heteroaryl then R^{a} is not —CF₃.

Exemplary Embodiment No. 3. The compound of Exemplary Embodiment 1 or Exemplary Embodiment 2, wherein: (ii) X¹ is CH or N; and R¹ and R², together with the atoms to which they are attached, form a pyridine optionally substituted with one or more R^{a}; or
(iii) X² is N or O; and R¹ and R², together with the atoms to which they are attached, form a pyridine optionally substituted with one or more R^{a}; or
(iv) when R¹ and R², together with the atoms to which they are attached, form a pyridine optionally substituted with one or more R^{a}, then X¹ is not S; or
(v) when R¹ and R², together with the atoms to which they are attached, form a pyridine optionally substituted with one or more R^{a}, then X² is not S; or
(vi) when R¹ and R², together with the atoms to which they are attached, form a pyridine optionally substituted with one or more R^{a}, then

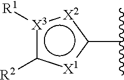

is not

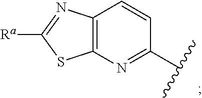

or
(vii) when R^{a} is

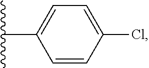 [SMILES: *c1ccc(Cl)cc1]

then R³ is not

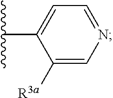

or
(viii) when R^{a} is

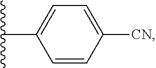 [SMILES: *c1ccc(C#N)cc1]

then R³ is not

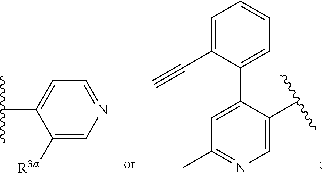

or
(ix) when R¹ and R², together with the atoms to which they are attached, form a pyrazine substituted with one or more R^{a}, and R^{a} is phenyl substituted with one or more R^{a1}, then R^{a1} is not —Cl, —F, —CN, —O(CH₃), —O(CF₃), —O(CHF₂), —CH₃, or cyclopropyl; or
(x) when R¹ and R², together with the atoms to which they are attached, form a pyrazine substituted with one or more R^{a}, and R^{a} is pyridine substituted with one or more R^{a1}, then R^{a1} is not —Cl, —CN, —CF₃, —O(CH₃), —O(CHF₂), cyclopropyl, —C(CH₃)₂(CN), or —C(CH₃)₂(OH); or
(xi) when R¹ and R², together with the atoms to which they are attached, form a pyrazine substituted with one or more R^{a}, then R^{a} is not

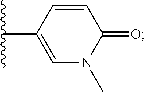 [SMILES: *c1ccc(=O)n(C)c1]

or
(xii) the compound of Formula (I) is not 5′-methoxy-2′,6-dimethyl-N-(6-(tetrahydrofuran-3-yl)thiazolo[4,5-b]pyrazin-2-yl)-[4,4′-bipyridine]-3-carboxamide.

Exemplary Embodiment No. 4. The compound of any one of the preceding Exemplary Embodiments, wherein X¹ is CH or N.

Exemplary Embodiment No. 5. The compound of any one of the preceding Exemplary Embodiments, wherein:
each R^{a} independently is oxo, halo, —C(O)N(R^{b})(R^{c}), —N(R^{b})C(O)(R^{c}), C₁-C₆ alkyl, C₁-C₆ haloalkyl, C₃-C₁₀ cycloalkyl, C₆-C₁₀ aryl, 5- to 10-membered heteroaryl, or 3- to 10-membered heterocycloalkyl, wherein the C₁-C₆ alkyl, C₁-C₆ haloalkyl, C₃-C₁₀ cycloalkyl, C₆-C₁₀ aryl, 5- to 10-membered heteroaryl, or 3- to 10-membered heterocycloalkyl is optionally substituted with one or more R^{a1};
each R^{a1} independently is oxo, halo, cyano, —OH, —O(C₃-C₁₀ cycloalkyl), —NH(C₁-C₆ alkyl), —N(C₁-C₆ alkyl)₂, —C(O)O(C₁-C₆ alkyl), C₁-C₆ alkyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl, wherein the C₁-C₆ alkyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl is optionally substituted with one or more R^{a2};
each R^{a2} independently is cyano or —OH;
each R^{3a} independently is cyano, C₁-C₆ alkyl, C₁-C₆ haloalkyl, 3- to 10-membered heterocycloalkyl, C₆-C₁₀ aryl, or 5- to 10-membered heteroaryl, wherein the C₁-C₆ alkyl, C₁-C₆ haloalkyl, C₃-C₁₀ cycloalkyl, 3- to 10-membered heterocycloalkyl, C₆-C₁₀ aryl, or 5- to 10-membered heteroaryl is optionally substituted with one or more R^{3a1}; and
each R^{3a1} independently is oxo, halo, cyano, —C(O)(C₁-C₆ alkyl), —C(O)NH₂, —C(O)(O—(C₁-C₆ alkyl)), C₁-C₆ alkyl optionally substituted with one or more C₃-C₁₀ cycloalkyl, C₁-C₆ alkoxy, —O(C₁-C₆ haloalkyl), C₃-C₁₀ cycloalkyl, or 5- to 10-membered heteroaryl.

Exemplary Embodiment No. 6. The compound of any one of the preceding Exemplary Embodiments, wherein R¹ and R², together with the atoms to which they are attached, form a C₆ aryl or 6-membered heteroaryl optionally substituted with one or more R^{a}.

Exemplary Embodiment No. 7. The compound of any one of the preceding Exemplary Embodiments, wherein R¹ and R², together with the atoms to which they are attached, form a 5-membered heteroaryl optionally substituted with one or more R^{a}

Exemplary Embodiment No. 8. The compound of any one of the preceding Exemplary Embodiments, wherein R¹ and R², together with the atoms to which they are attached, form pyrazinyl, pyridinyl, or diazolyl, wherein the pyrazinyl, pyridinyl, or diazolyl is optionally substituted with one or more R^{a}.

Exemplary Embodiment No. 9. The compound of any one of the preceding Exemplary Embodiments, wherein each R^{a} independently is: oxo, —Cl, —Br, —CH₃, —CF₃

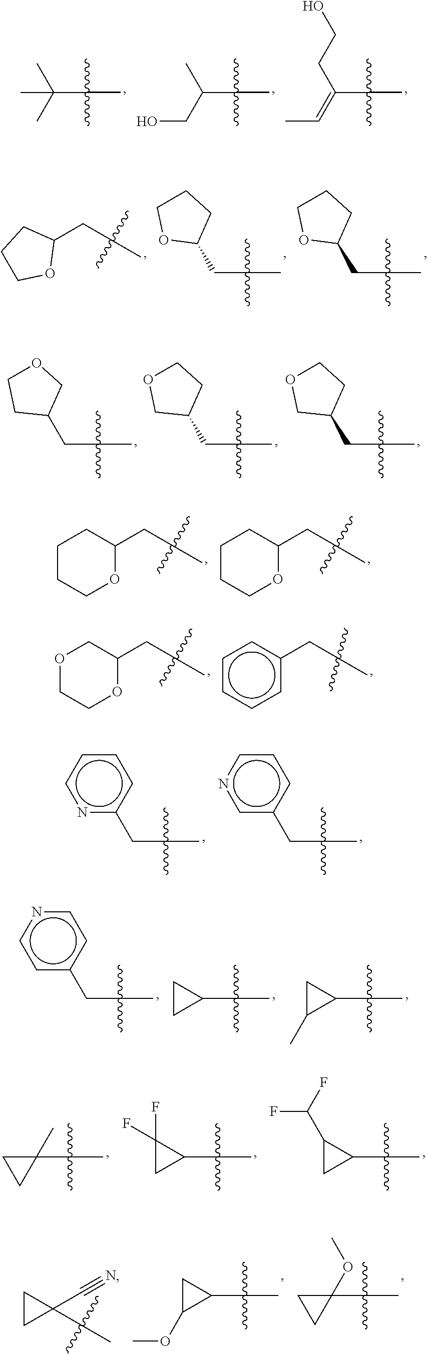

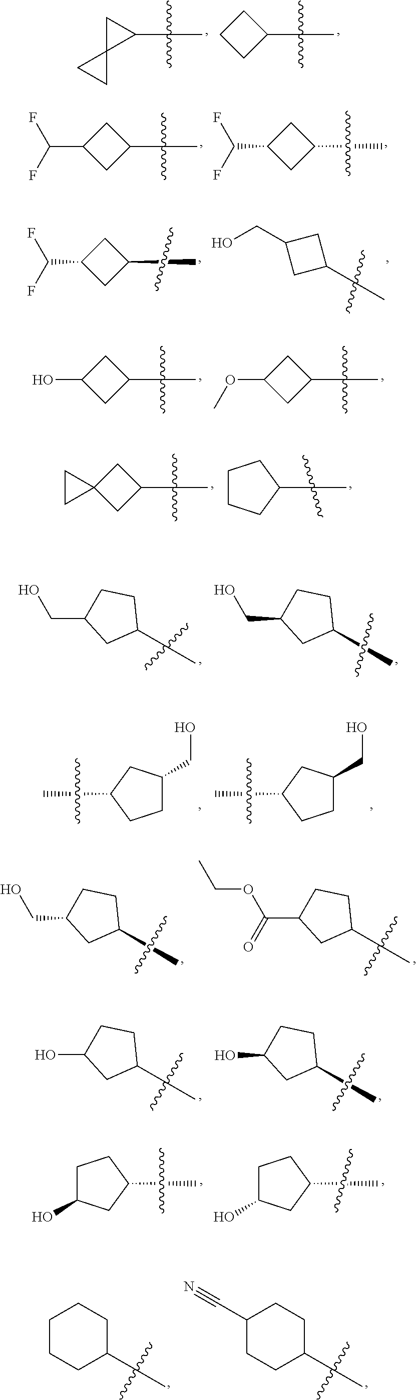

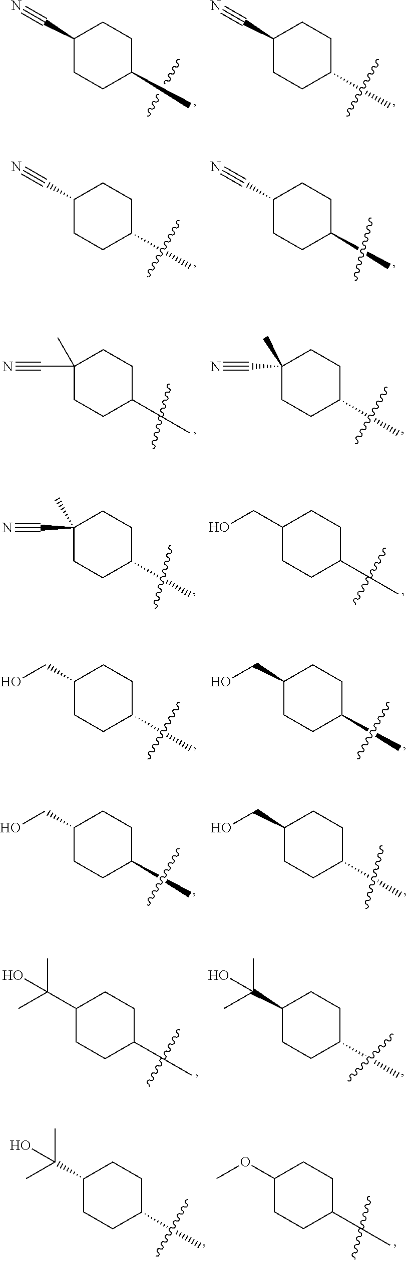

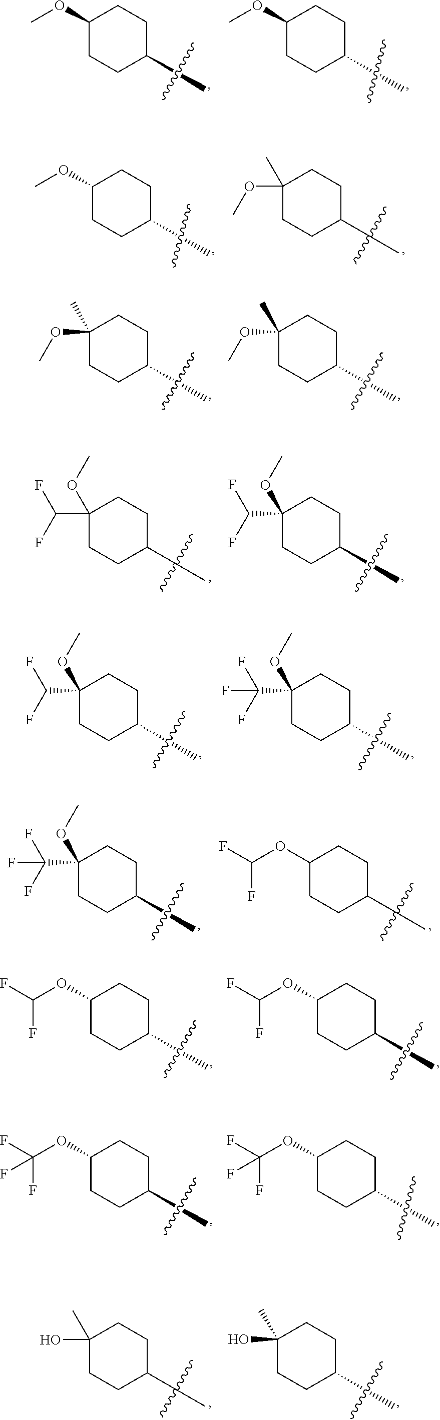

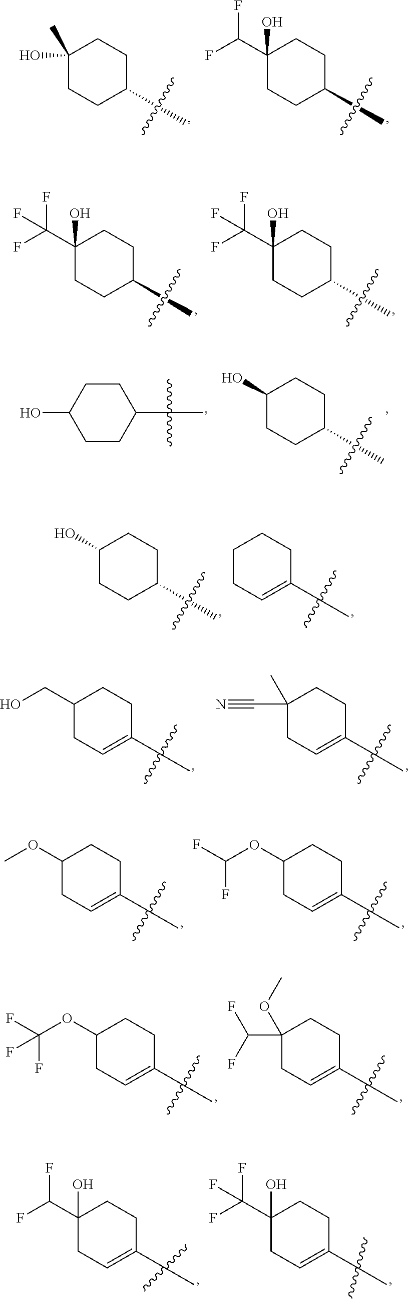

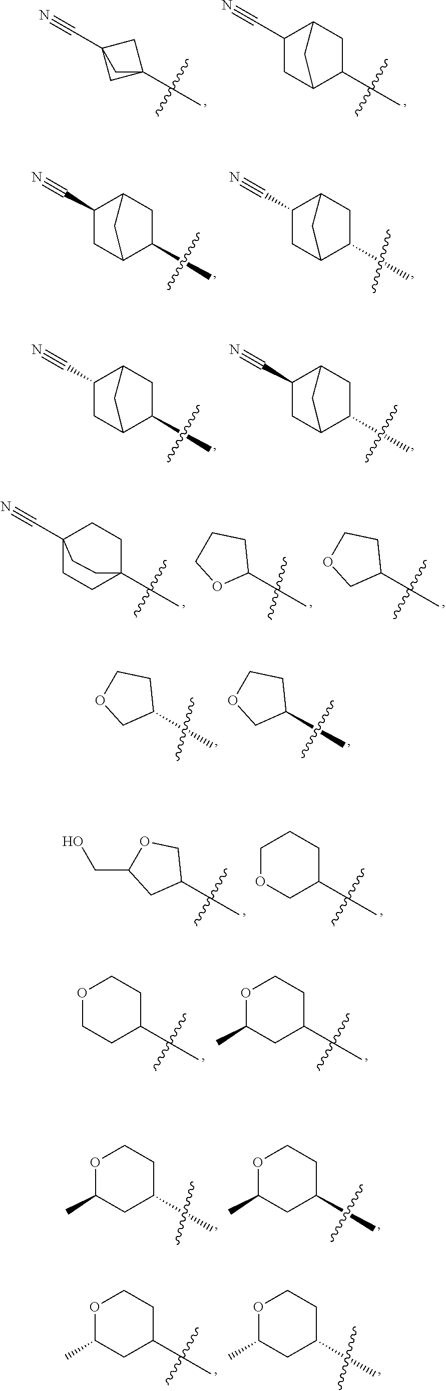

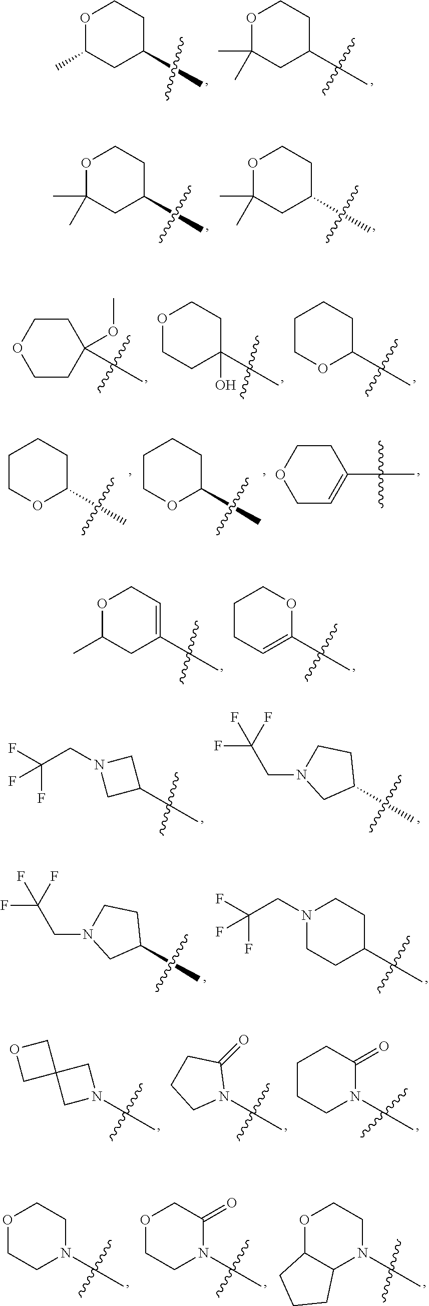

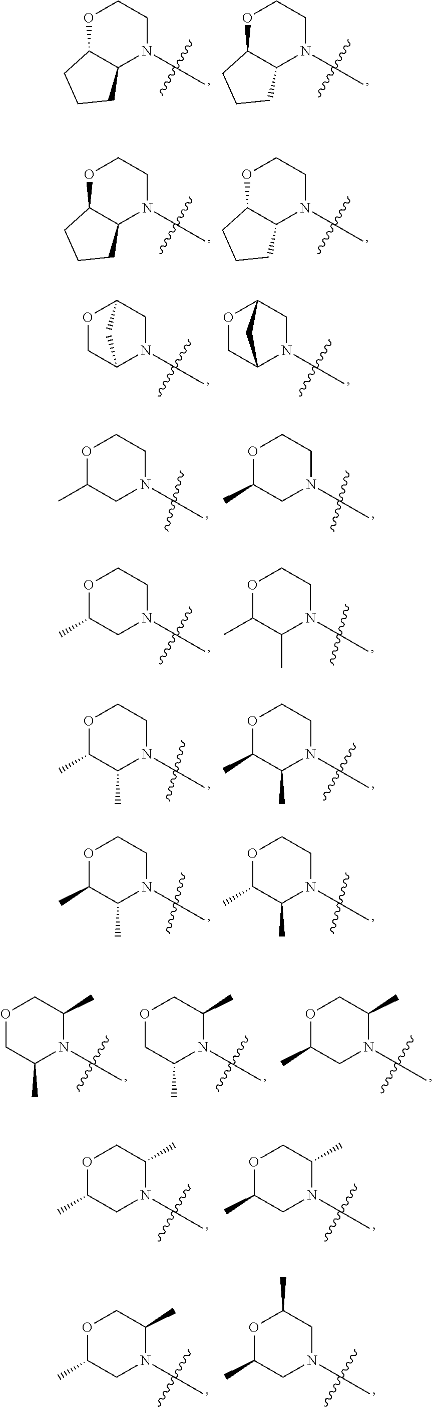

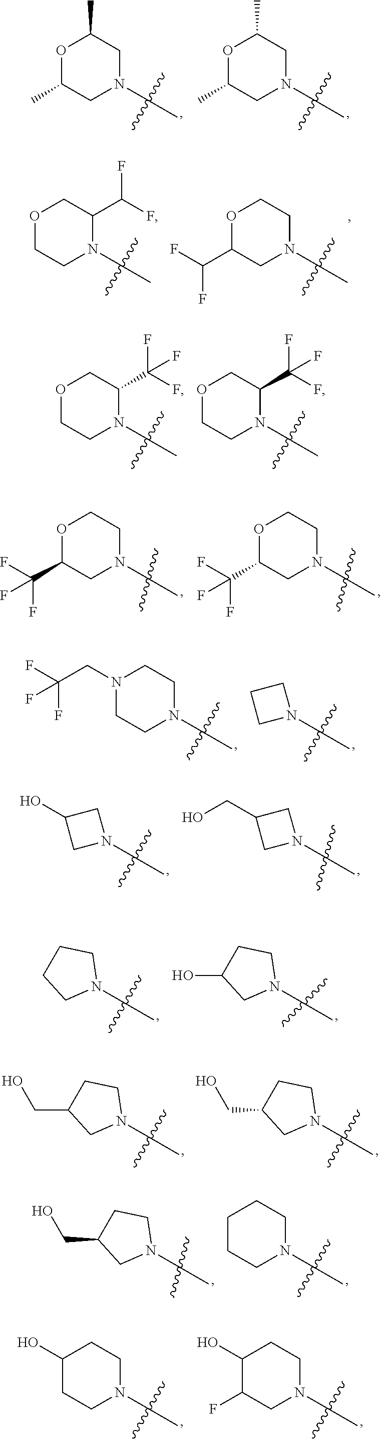

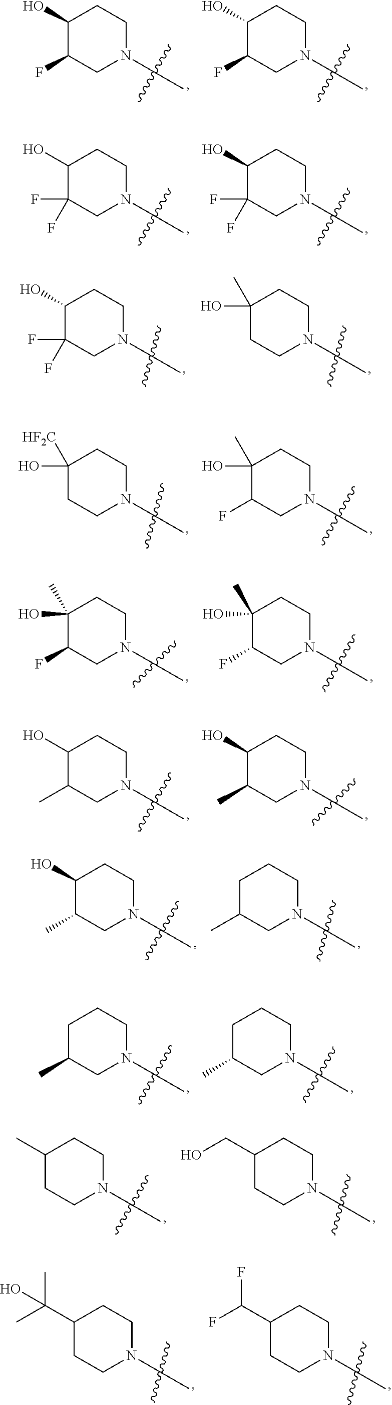

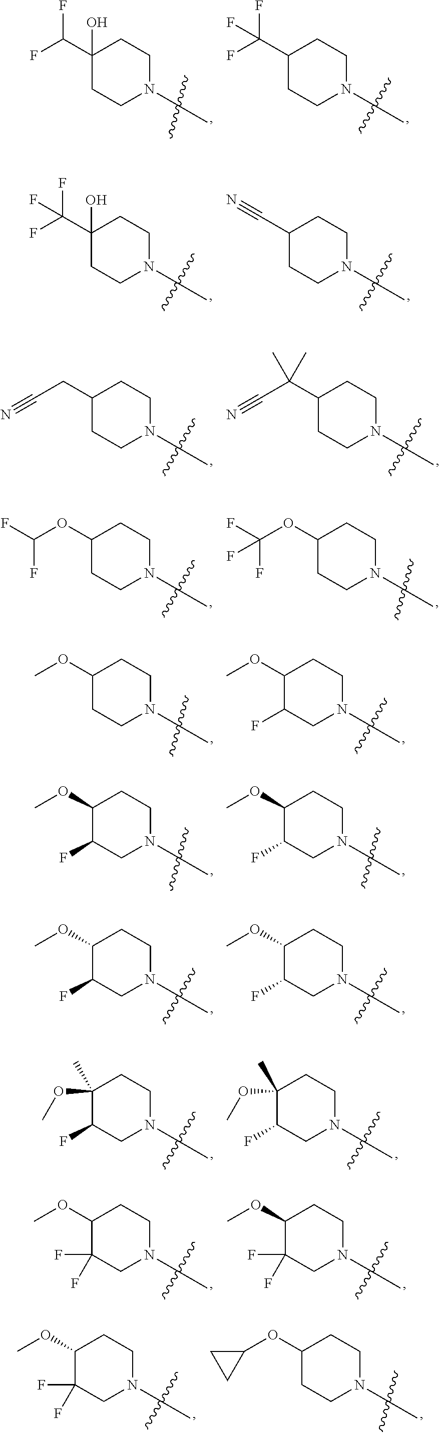

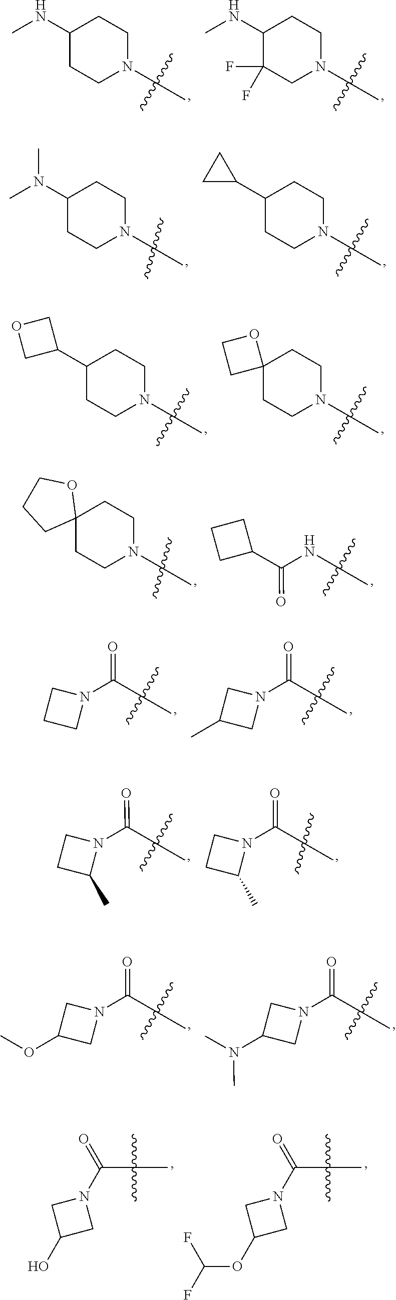

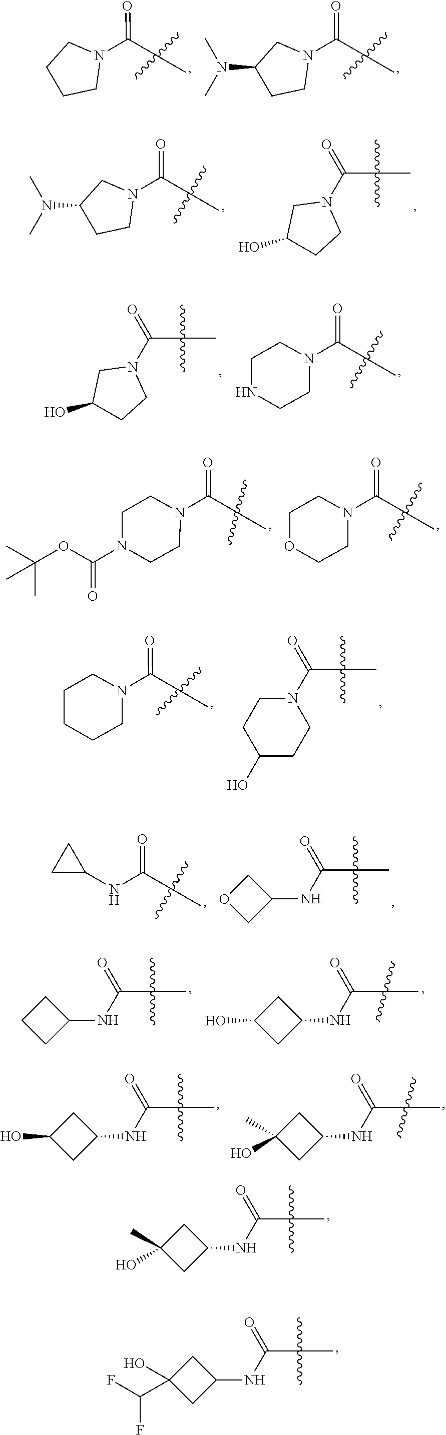

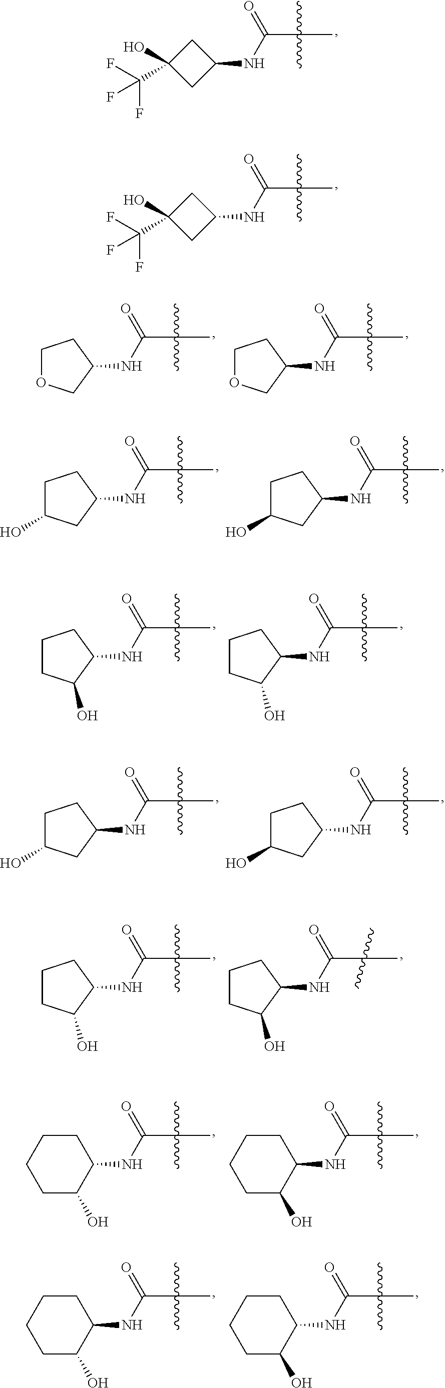

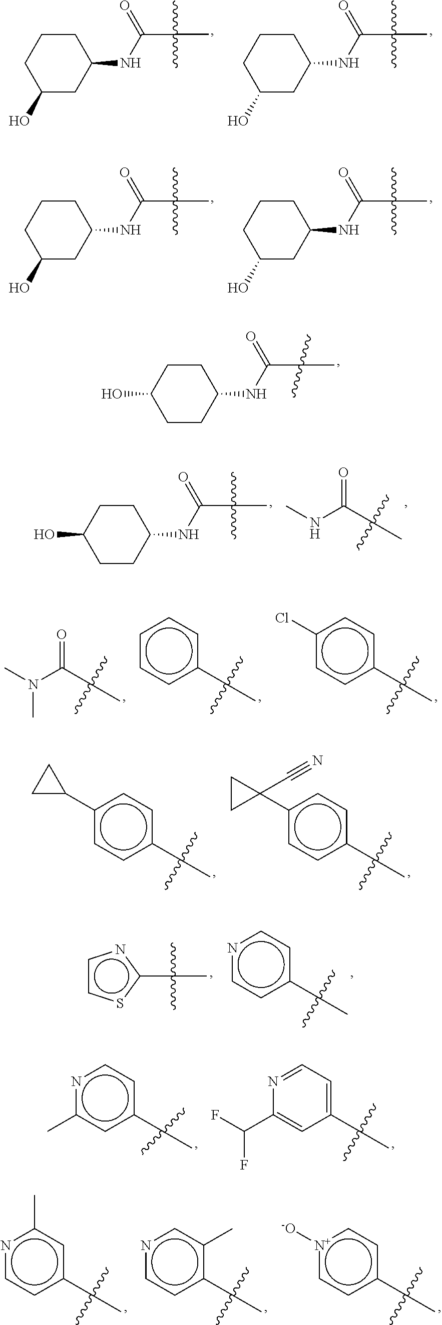

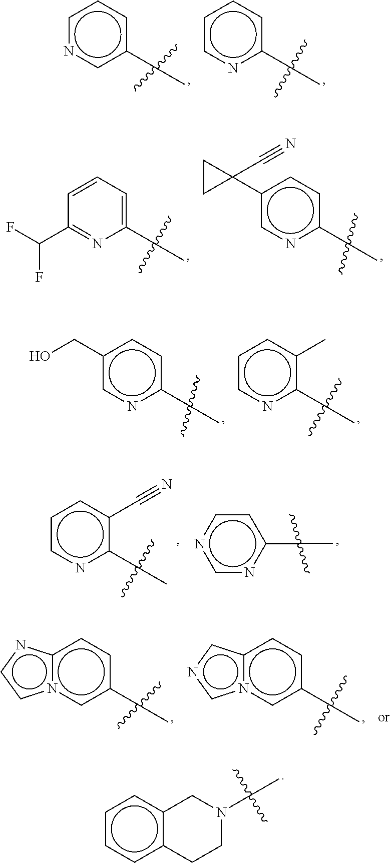

Exemplary Embodiment No. 10. The compound of any one of the preceding Exemplary Embodiments, wherein each R^{b} and Re independently is H, methyl, ethyl, cyclobutyl, cyclopropyl,

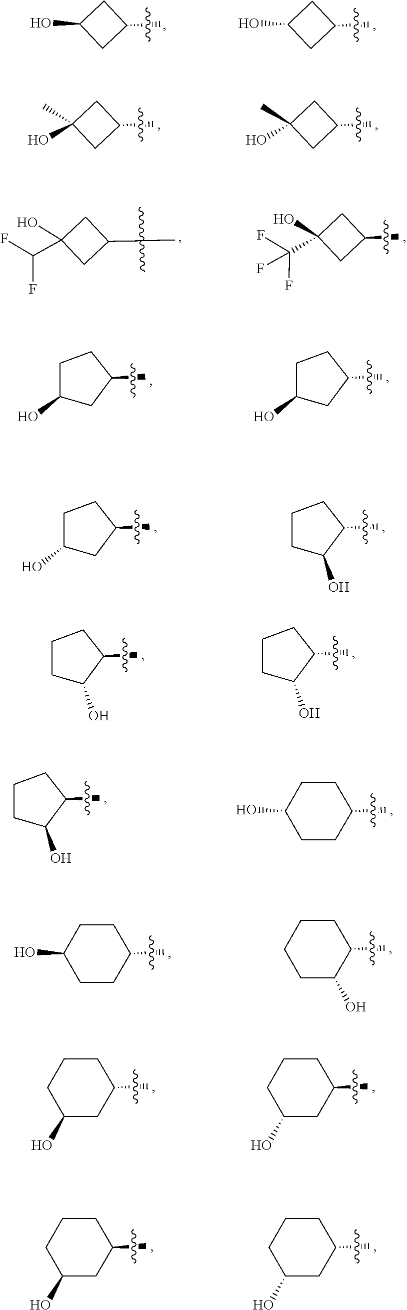

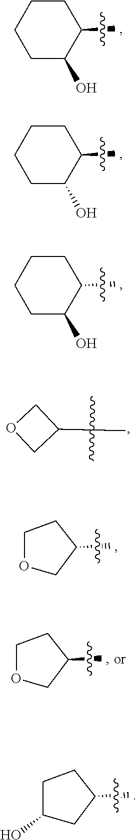

Exemplary Embodiment No. 11. The compound of any one of the preceding Exemplary Embodiments, wherein R^{b} and R, together with the atoms to which they are attached, form a heterocycloalkyl selected from:

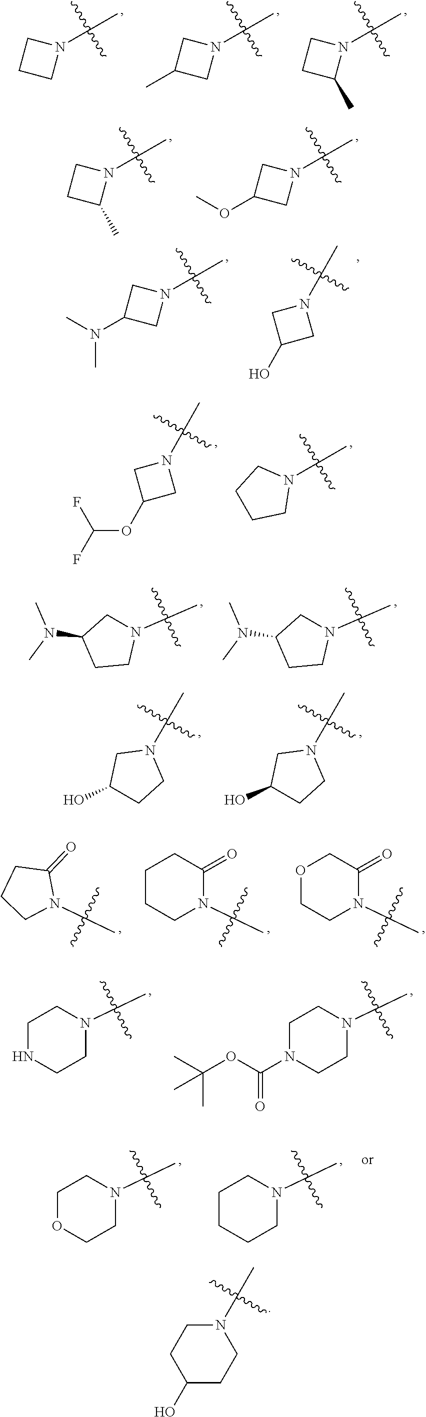

Exemplary Embodiment No. 12. The compound of any one of the preceding Exemplary Embodiments, wherein R³ is:

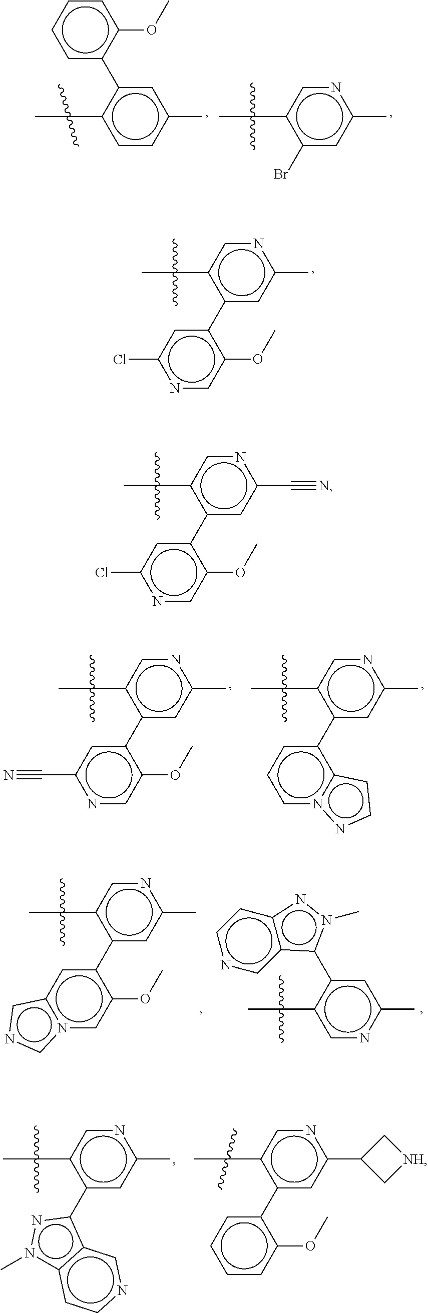

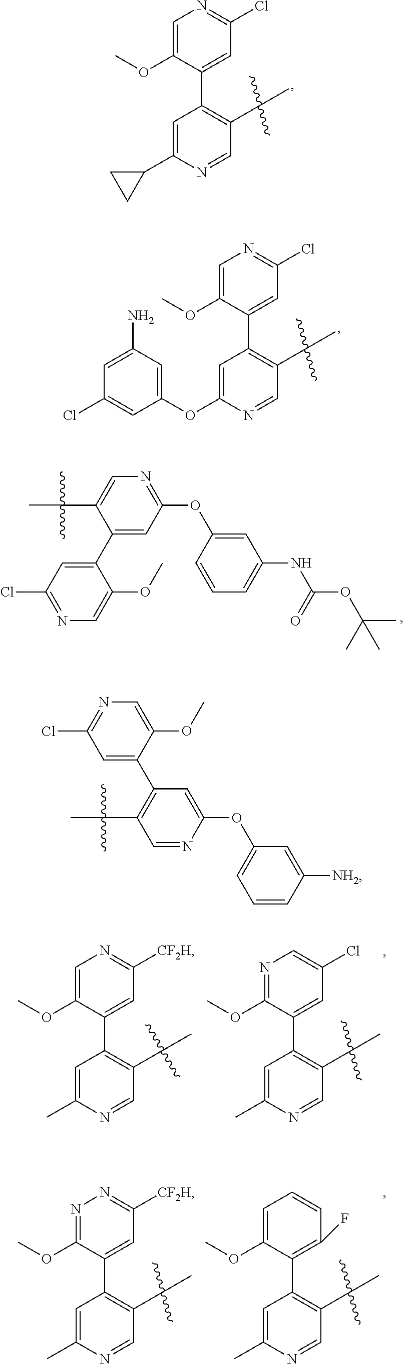

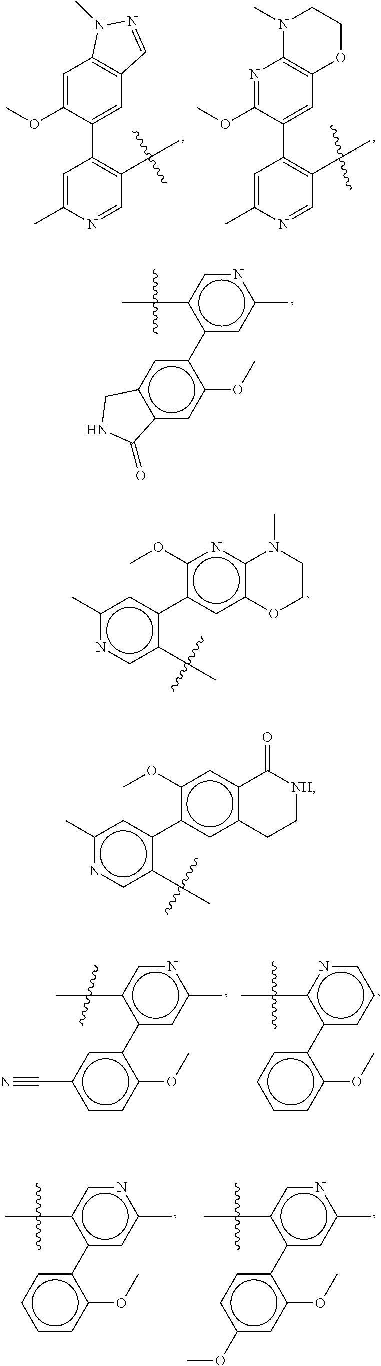

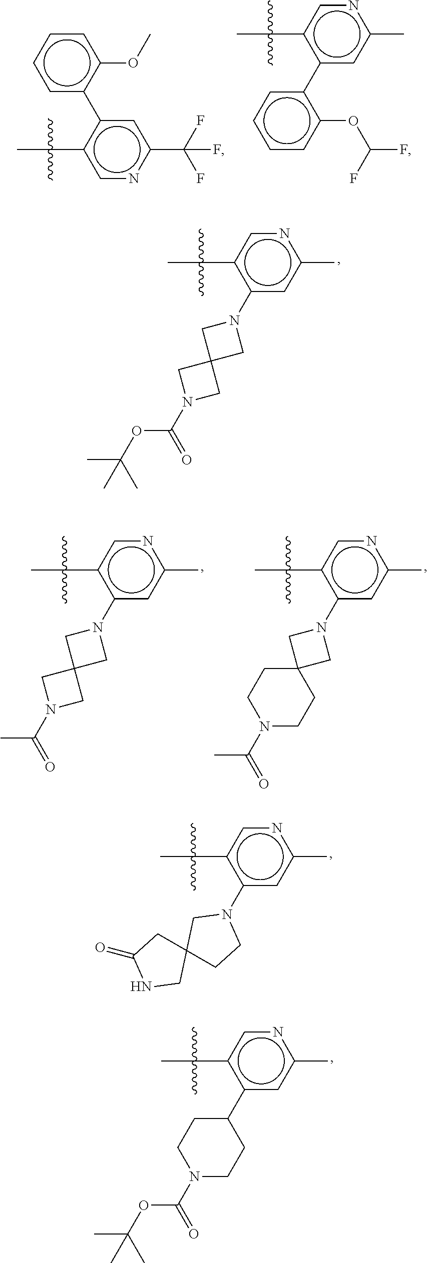

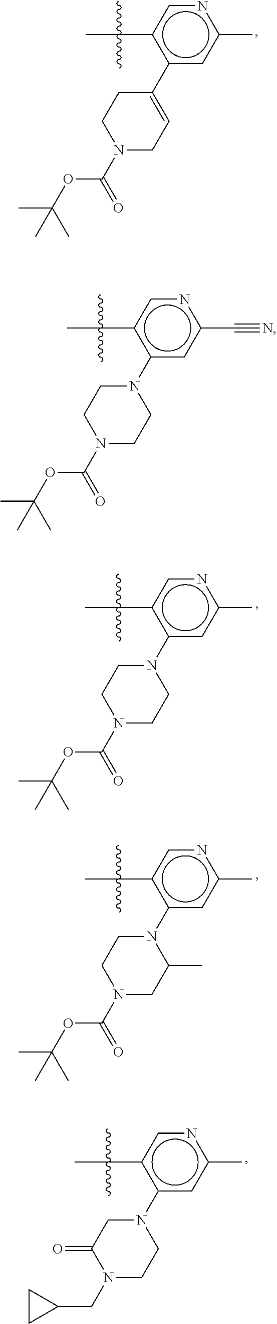

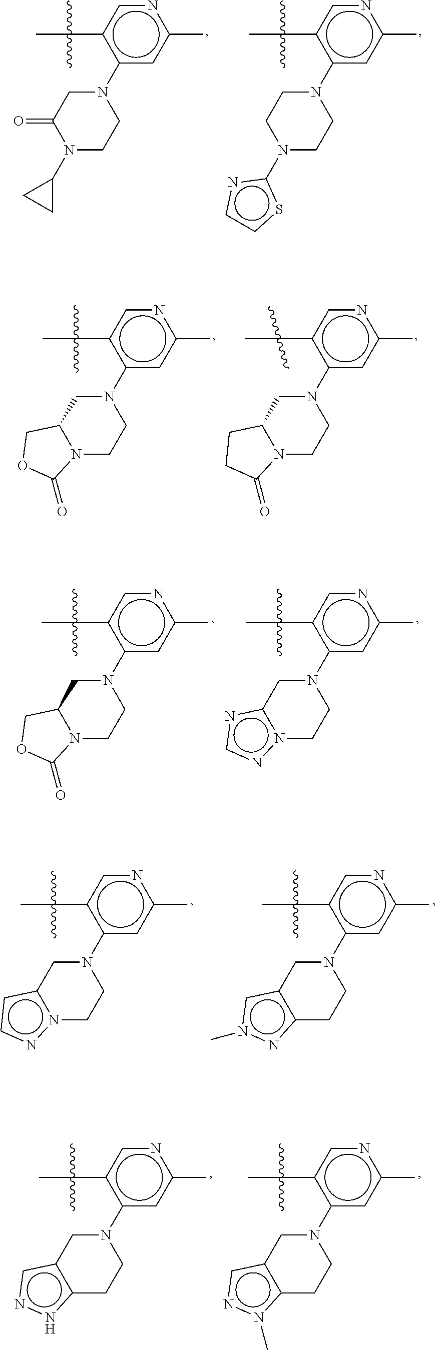

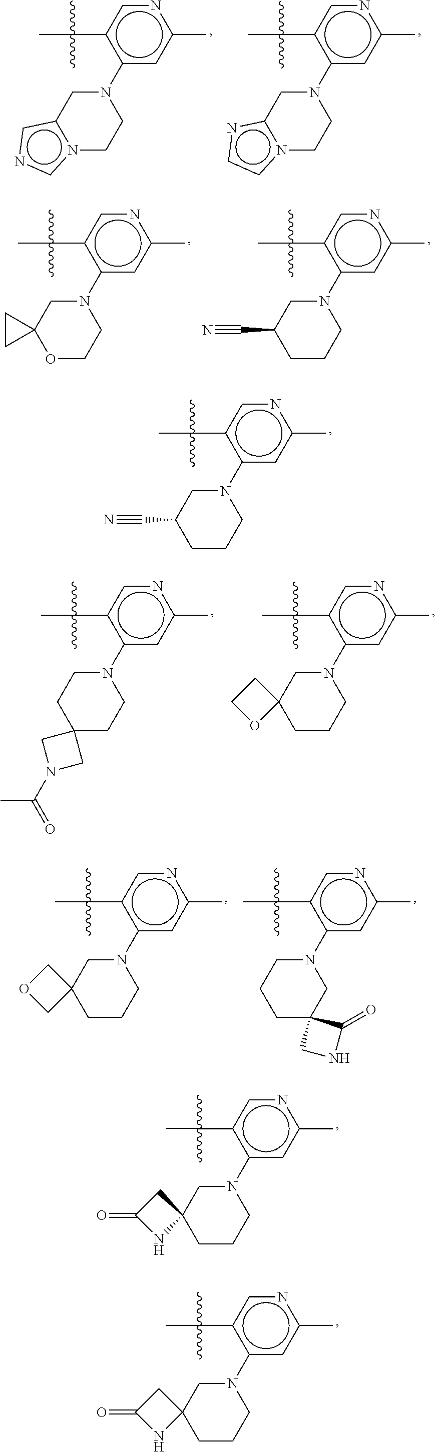

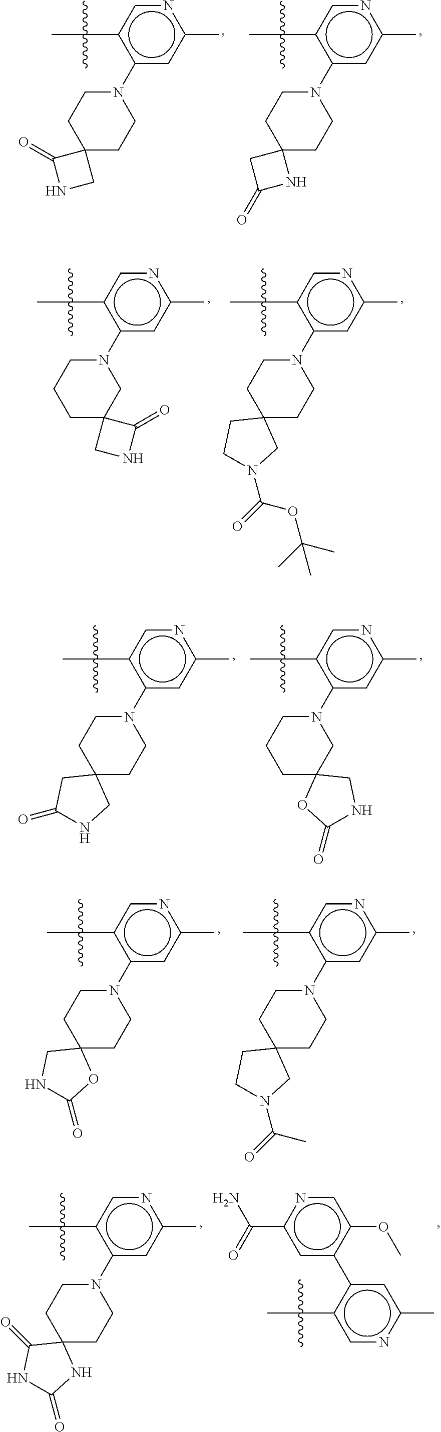

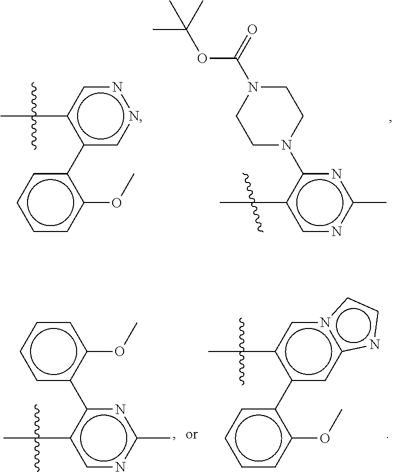

Exemplary Embodiment No. 13. The compound of any one of the preceding Exemplary Embodiments, wherein each R^{a1} independently is oxo, —OH, —CH₂OH, —CHF₂, —CF₃, —F, —Cl, —CN, —OCH₃, —OCHF₂, —OCF₃, —NHCH₃, —N(CH₃)₂, —CH₃, —CH₂CF₃, —CH₂CN, O

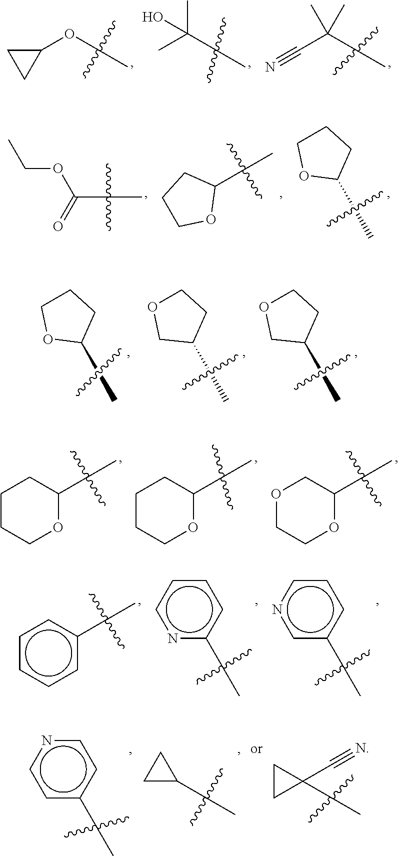

Exemplary Embodiment No. 14. The compound of any one of the preceding Exemplary Embodiments, wherein each R^{3a} independently is —Br, —CH₃, —CF₃, —CN,

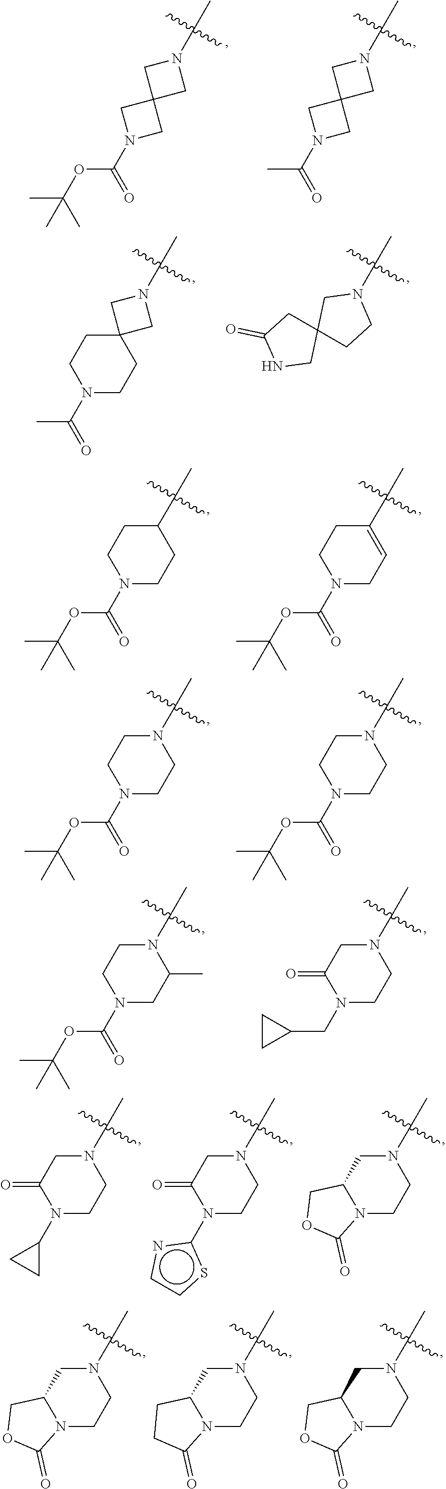

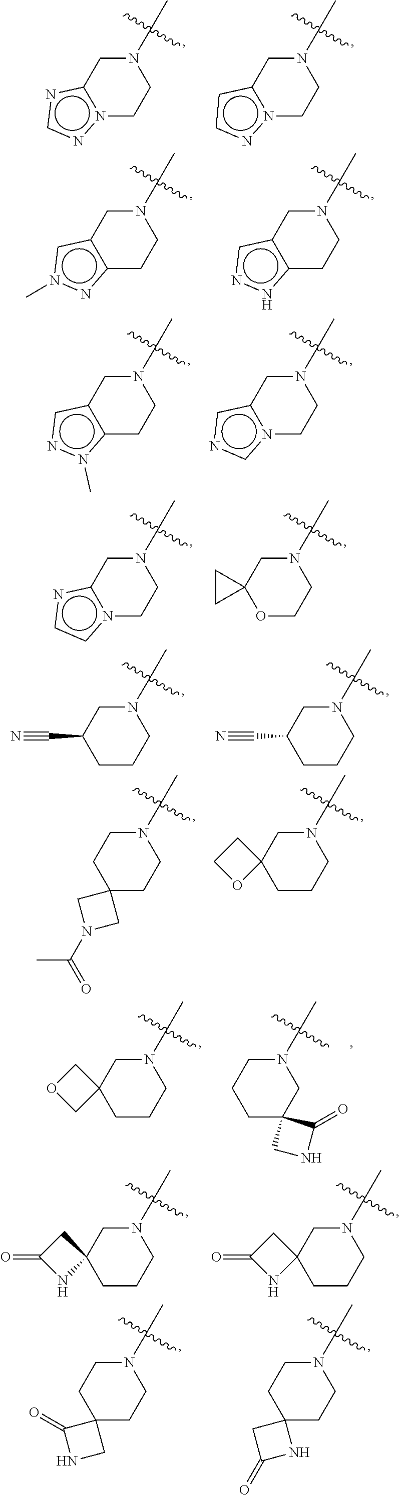

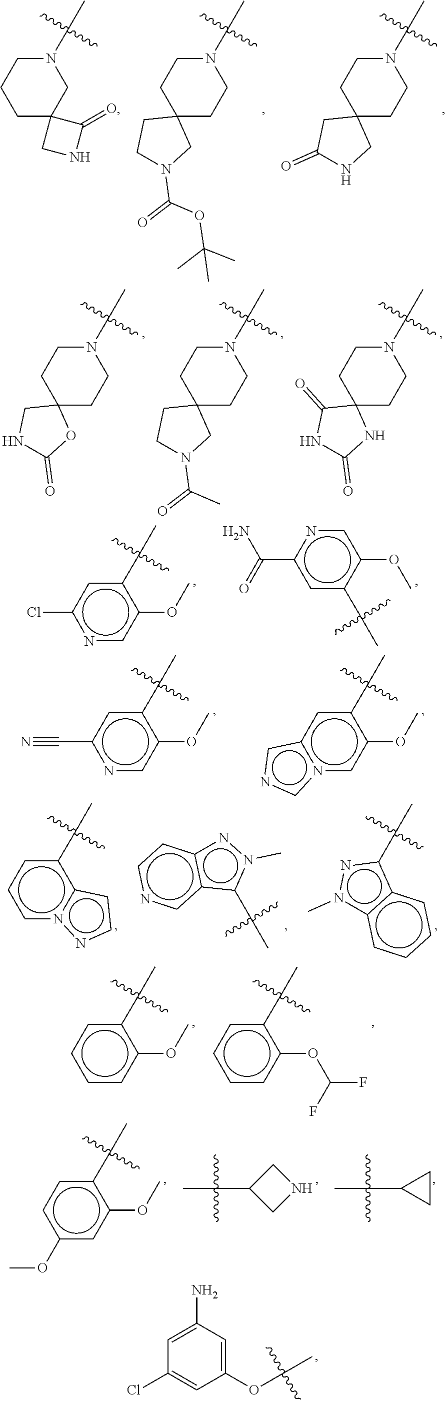

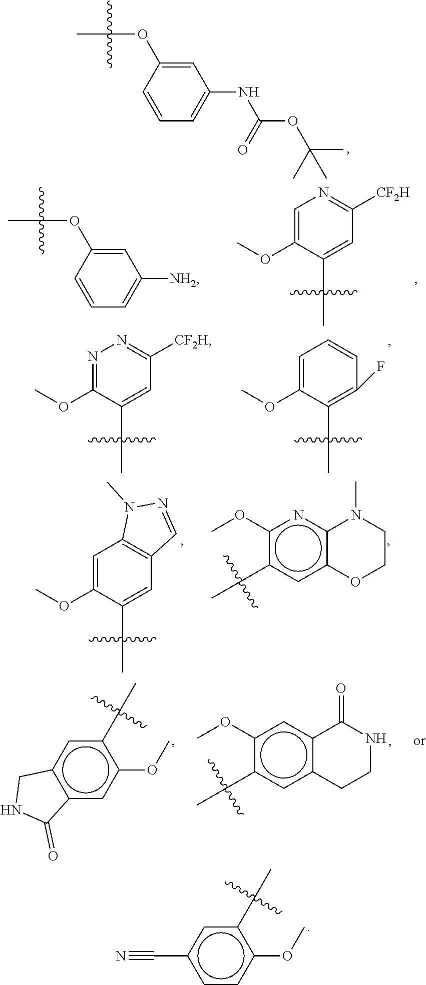

Exemplary Embodiment No. 15. The compound of any one of the preceding Exemplary Embodiments, wherein each R^{3a1} independently is oxo, —Cl, —CH₃, —OCH₃, —CF₂H, —NH₂, —NHC(O)OC(CH₃)₃, —OCHF₂, —C(O)OC(CH₃)₃, —C(O)CH₃, —C(O)NH₂, —CN,

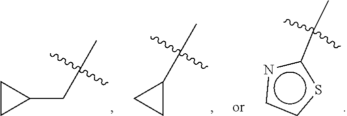

Exemplary Embodiment No. 16. The compound of any one of the preceding Exemplary Embodiments, wherein each R^{a2} independently is cyano.

Exemplary Embodiment No. 17. The compound of any one of the preceding Exemplary Embodiments, wherein the compound is of Formula (I-A), (I-B), or (I-C):

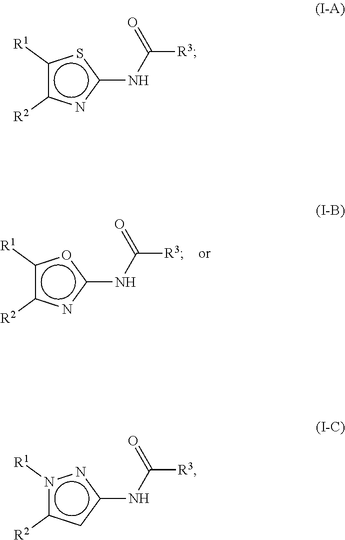

or a pharmaceutically acceptable salt, solvate, clathrate, hydrate, stereoisomer, or tautomer thereof.

Exemplary Embodiment No. 18. The compound of any one of Exemplary Embodiments 1-6, wherein the compound is of Formula (I-D), (I-E), (I-F), (I-G), (I-H), (I-I), (I-J), (I-K) or (I-L):

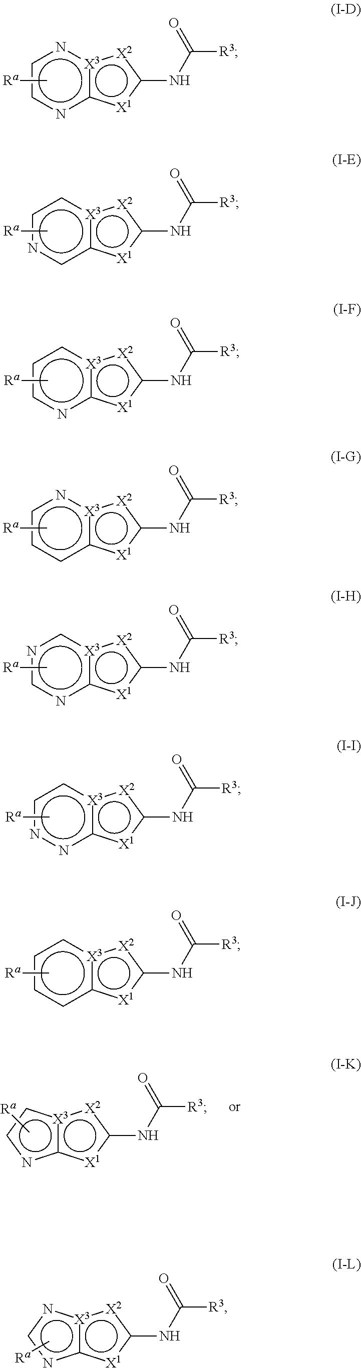

or a pharmaceutically acceptable salt, solvate, clathrate, hydrate, stereoisomer, or tautomer thereof.

Exemplary Embodiment No. 19. The compound of any one of Exemplary Embodiments 1-6, wherein the compound is of Formula (I-F′):

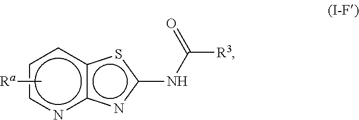

or a pharmaceutically acceptable salt, solvate, clathrate, hydrate, stereoisomer, or tautomer thereof.

Exemplary Embodiment No. 20. The compound of any one of Exemplary Embodiments 1-6, wherein the compound is of Formula (I-M), (I-N), (I-O), (I-P), (I-Q), (I-R), (I-S), (I-T), (I-U), or (I-V):

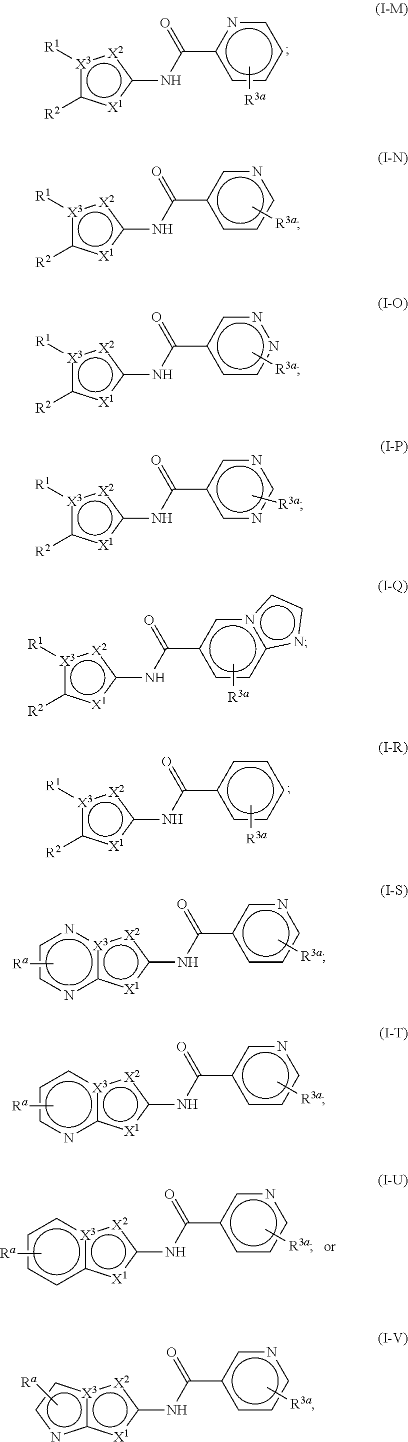

or a pharmaceutically acceptable salt, solvate, enantiomer, diastereomer, racemate or mixture thereof.

Exemplary Embodiment No. 21. The compound of any one of Exemplary Embodiments 1-6, wherein the compound is of Formula (I-T′):

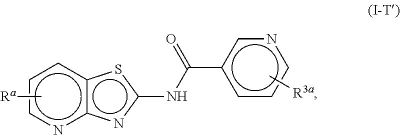

or a pharmaceutically acceptable salt, solvate, enantiomer, diastereomer, racemate or mixture thereof.

Exemplary Embodiment No. 22. The compound of any one of Exemplary Embodiments 1-6, wherein the compound is of Formula (I-W):

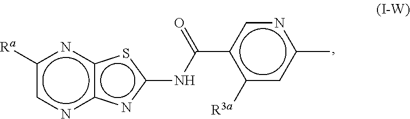

or a pharmaceutically acceptable salt, solvate, enantiomer, diastereomer, racemate or mixture thereof.

Exemplary Embodiment No. 23. The compound of any one of the preceding Exemplary Embodiments, wherein the compound is selected from a compound described in Tables 1-4, or a pharmaceutically acceptable salt thereof.

Exemplary Embodiment No. 24. The compound of any one of the preceding Exemplary Embodiments, wherein the compound is selected from Compound Nos. 1, 10, 24, 25, 59, 207, 211, 212, 224, 235, 238, 239, 244, 3, 12, 280, 282, 288, 308, 359, 360, 361B, 368, 377, 386, 391, 394, 403, 427, 432, 447, 451A, 454, 462, 464, 467, 486, 489, 498, 502, 504, 506, 514, 518, 520, 521, and 522, or a pharmaceutically acceptable salt thereof.

Exemplary Embodiment No. 25. A compound obtainable by, or obtained by, a method described herein; optionally, the method comprises one or more steps described in Schemes 1-11.

Exemplary Embodiment No. 26. A pharmaceutical composition comprising the compound of any one of the preceding Exemplary Embodiments or a pharmaceutically acceptable salt thereof, and a pharmaceutically acceptable diluent or carrier.

Exemplary Embodiment No. 27. The pharmaceutical composition of Exemplary Embodiment 26, wherein the compound is selected from a compound described in Tables 1-4.

Exemplary Embodiment No. 28. A method of modulating DNA polymerase Θ activity, comprising contacting a cell with a compound of any one of Exemplary Embodiments 1-25 or a pharmaceutical composition of Exemplary Embodiment 26 or Exemplary Embodiment 27.

Exemplary Embodiment No. 29. A method of treating or preventing a disease or disorder in a subject in need thereof, comprising administering to the subject a compound of any one of Exemplary Embodiments 1-25 or pharmaceutical composition of Exemplary Embodiment 26 or Exemplary Embodiment 27.

Exemplary Embodiment No. 30. The compound of any one of Exemplary Embodiments 1-25 or pharmaceutical composition of Exemplary Embodiment 26 or Exemplary Embodiment 27 for use in modulating DNA polymerase Θ activity.

Exemplary Embodiment No. 31. The compound of any one of Exemplary Embodiments 1-25 or pharmaceutical composition of Exemplary Embodiment 26 or Exemplary Embodiment 27 for use in treating or preventing a disease or disorder.

Exemplary Embodiment No. 32. Use of the compound of any one of Exemplary Embodiments 1-25 in the manufacture of a medicament for modulating DNA polymerase Θ activity.

Exemplary Embodiment No. 33. Use of the compound of any one of Exemplary Embodiments 1-25 in the manufacture of a medicament for treating or preventing a disease or disorder.

Exemplary Embodiment No. 34. The method, compound, pharmaceutical composition, or use of any one of Exemplary Embodiments 28-33 wherein the disease or disorder is associated with an implicated DNA polymerase Θ activity.

Exemplary Embodiment No. 35. The method, compound, pharmaceutical composition, or use of any one of Exemplary Embodiments 28-34, wherein the disease or disorder is cancer.

### EXAMPLES

For exemplary purposes, neutral compounds of Formula (I) are synthesized and tested in the examples. It is understood that the neutral compounds of Formula (I) may be converted to the corresponding pharmaceutically acceptable salts of the compounds using routine techniques in the art (e.g., by saponification of an ester to the carboxylic acid salt, or by hydrolyzing an amide to form a corresponding carboxylic acid and then converting the carboxylic acid to a carboxylic acid salt).

### Abbreviations

ACN or MeCN Acetonitrile
AcOH Acetic Acid
BFMO N1,N2-Bis(furan-2-ylmethyl)oxalamide
Boc tert-Butoxycarbonyl
(Boc)₂O Di-tert-butyl dicarbonate
dba Tris(dibenzylideneacetone)
DAST Diethylaminosulfur trifluoride
DCE 1,2-Dichloroethane
DCM Dichloromethane
DIC N,N′-Diisopropylcarbodiimide
DIPEA or DIEA Diisopropylethylamine
DMA N,N-Dimethylacetamide
DMAP 4-Dimethylaminopyridine
DME Dimethoxyethane
DMF Dimethylformamide
DMSO Dimethyl sulfoxide
dppf 1,1′-bis(diphenylphosphino)ferrocene
dtpby 1,1′-bis(di-tert-butylphosphino)ferrocene
(dtpby)NiBr₂ [4,4′-Bis(1,1-dimethylethyl)-2,2′-bipyridine]nickel (II) dichloride
EDCI-HCl N-Ethyl-N′-(3-dimethylaminopropyl)carbodiimide hydrochloride
ESI Electrospray ionization
EtOAc Ethyl acetate
EtOH Ethanol
FA Formic acid
g Gram
h Hours
HATU Hexafluorophosphate azabenzotriazole tetramethyl uronium
HCl Hydrochloric acid
HOBt 1-Hydroxybenzotriazole hydrate
HPLC High-performance liquid chromatography
Hz Hertz
[Ir(dtbbpy)(ppy)₂][PF₆][4,4′-Bis(1,1-dimethylethyl)-2,2′-bipyridine-N1,N1′]bis[2-(2-pyridinyl-N)phenyl-C]iridium(III) hexafluorophosphate
K₂CO₃ Potassium carbonate
KI Potassium iodide
KOAc Potassium acetate
L Liter
LDA Lithium Diisopropylamide
LCMS Liquid chromatography-mass spectrometry
LiOH Lithium hydroxide
M Molar
m-CPBA meta-Chloroperoxybenzoic acid
Mn(dpm)₃ Tris(2,2,6,6-tetramethyl-3,5-heptanedionato)manganese(III)
m/z Mass to charge ratio
MeOH Methanol
mg Milligram
MHz Megahertz
min Minutes
mL Milliliter
mmol Millimolar
MPa Megapascal
MsCl Methanesulfonyl chloride
MTBE Methyl tert-butyl ether
NaBH₄ Sodium borohydride
NH₄Cl Ammonium chloride
NaHMDS Sodium bis(trimethylsilyl)amide
NaOEt Sodium ethoxide
NaOH Sodium hydroxide
Na₂SO₄ Sodium sulfate
NCS N-Chlorosuccinimide
NIS N-Iodosuccinimide
nm nanometer
NMI 1-methylimidazole
NMP 1-Methyl-2-pyrrolidone
NMR Nuclear magnetic resonance
PCy₃ Tricyclohexylphosphine
Pd-PEPPSI™ iPent [1,3-Bis(2,6-Di-3-pentylphenyl)imidazol-2-ylidene](3-chloropyridyl)palladium(II) dichloride
Pet ether Petroleum ether
Pin₂B₂ Bis(pinacolato)diboron
PPh₃ Triphenylphosphine
(PPh₃)₄ Tetrakis(triphenylphosphine)
Prep Preparative
PyBop Benzotriazol-1-yloxytripyrrolidinophosphonium hexafluorophosphate
PtO₂ Platinum oxide
RT Room temperature
SFC Supercritical fluid chromatography
SOCl₂ Thionyl chloride
t-BuOK Potassium tert-butoxide
T3P Propanephosphonic acid anhydride
TBAF Tetrabutylammonium fluoride
TBAI Tetrabutylammonium iodide
TBSCl tert-Butyldimethylsilyl chloride
TCFH N′-tetramethylformamidinium hexafluorophosphate
TEA Triethylamine
Tf₂O Triflic anhydride
TFA Triflluoroacetic acid
THF Tetrahydrofuran
TLC Thin layer chromatography
TMSCHN₂ Trimethylsilyldiazomethane
TMSCl Trimethylsilyl chloride
wt Weight
Xantphos 9,9-Dimethyl-9H-xanthene-4,5-diyl)bis(diphenylphosphane)
X-phos 2-Dicyclohexylphosphino-2′,4′,6′-triisopropylbiphenyl

Prep-HPLC conditions (unless otherwise stated): SHIMADZU Preparative HPLC System—including a LC-20AP Pump, a SPD-20A Detector and Labsolutions (version 5.90) software. Column: Agilent 10, Prep-C18, 250×21.2 mm. Solvent/Gradient: 5-80% Acetonitrile in water containing 0.10% HCOOH. Flow Rate: 20 mL/min.

### SYNTHESIS OF INTERMEDIATES

### Intermediate 1: 4-(2-Methoxyphenyl)-6-methylnicotinic acid

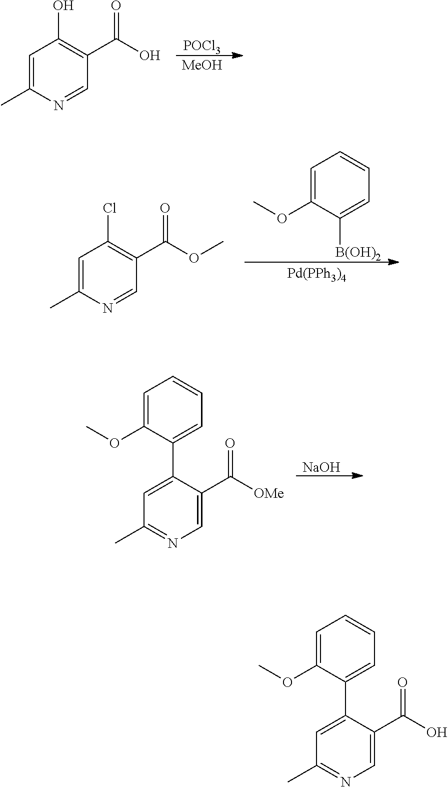

Step 1: A mixture of 4-hydroxy-6-methylnicotinic acid (10.0 g, 65.0 mmol) and POCl₃ (40 mL) was heated at reflux for 2 h then concentrated under reduced pressure. The residue was cooled to 0° C. and MeOH (40 mL) was added slowly dropwise. The mixture was allowed to warm to room temperature and stirred overnight. The mixture was adjusted to pH 7 with solid Na₂CO₃, diluted with water (500 mL) and extracted with DCM (3×300 mL). The combined organic layers were washed with water (500 mL), brine (500 mL), dried over Na₂SO₄, filtered and concentrated under reduced pressure. The residue was purified by silica gel chromatography (6.6-12.5% Pet. ether in EtOAc) to give methyl 4-chloro-6-methylnicotinate as a colorless oil (10.4 g, 85%). LCMS (ESI, m/z): 186 [M+H]⁺, ¹H NMR (400 MHz, DMSO-d₆) δ 8.84 (s, 1H), 7.57 (s, 1H), 3.87 (s, 3H), 2.52 (s, 3H).

Step 2: To a solution of methyl 4-chloro-6-methylnicotinate (7.00 g, 37.7 mmol) in dry 1,4-dioxane (80 mL) was added (2-methoxyphenyl)boronic acid (5.73 g, 37.7 mmol), Pd(PPh₃)₄ (2.18 g, 1.88 mmol) and Cs₂CO₃ (36.9 g, 113 mmol) and the mixture was heated at 85° C. overnight. The mixture was diluted with water (100 mL), extracted with EtOAc (3×300 mL) and the combined organic layers washed with water (100 mL), brine (100 mL), dried over Na₂SO₄, filtered and concentrated under reduced pressure. The residue was purified by silica gel chromatography (6.6-25% Pet. ether in EtOAc) to give methyl 4-(2-methoxyphenyl)-6-methylnicotinate (8.0 g, 82%) as a yellow oil. LCMS (ESI, m/z): 258 [M+H]⁺, ¹H NMR (400 MHz, DMSO-d₆) δ 8.73 (s, 1H), 7.40 (td, J=7.8, 1.8 Hz, 1H), 7.30-7.24 (m, 2H), 7.05 (t, J=8.0 Hz, 2H), 3.65 (s, 3H), 3.62 (s, 3H), 2.55 (s, 3H).

Step 3: To a solution of methyl 4-(2-methoxyphenyl)-6-methylnicotinate (8.0 g, 31.0 mmol) in water (40 mL) and MeOH (40 mL) was added NaOH (3.73 g, 93.0 mmol) and the mixture was heated at 50° C. for 2 h. The mixture was adjusted to pH 5 with 1 M aqueous HCl, extracted with a 10:1 mixture of DCM/MeOH (30 mL×10) and the combined organic layers were dried over Na₂SO₄, filtered and concentrated under reduced pressure to give 4-(2-methoxyphenyl)-6-methylnicotinic acid (5.5 g, 73%) as a white solid. LCMS (ESI, m/z): 244 [M+H]⁺, ¹H NMR (400 MHz, DMSO-d₆) δ 8.75 (s, 1H), 7.37 (td, J=7.8, 1.8 Hz, 1H), 7.26-7.16 (m, 2H), 7.03 (d, J=7.6 Hz, 2H), 3.67 (s, 3H), 2.53 (s, 3H).

### Intermediate 2: 4-(5-Cyano-2-methoxyphenyl)-6-methylnicotinic acid

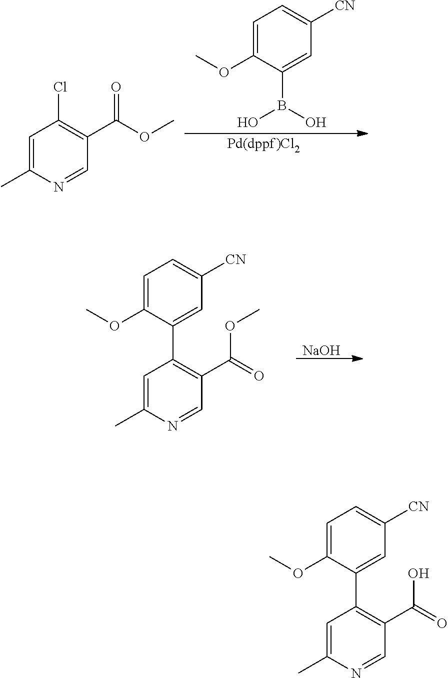

Step 1: A solution of methyl 4-chloro-6-methylnicotinate (5.2 g, 28 mmol), (5-cyano-2-methoxyphenyl)boronic acid (5.0 g 28 mmol), Pd(dppf)Cl₂ (2.0 g, 2.8 mmol) and Na₂CO₃ (9.0 g, 85 mmol) in 1,4-dioxane (200 mL) and water (40 mL) was heated at reflux overnight. The mixture was diluted with water (200 mL), extracted with EtOAc (2×200 mL) and the combined organic layers washed with brine (2×200 mL), dried over Na₂SO₄, filtered and concentrated under reduced pressure. The residue was purified by silica gel chromatography (Pet. ether/EtOAc=5/1, v/v) to give methyl 4-(5-cyano-2-methoxyphenyl)-6-methylnicotinate (5.0 g, 63%) as a yellow solid. LCMS (ESI, m/z): 283 [M+H]⁺.

Step 2: To a solution of methyl 4-(5-cyano-2-methoxyphenyl)-6-methylnicotinate (5.0 g, 18 mmol) in MeOH (200 mL) and water (400 mL) was added NaOH (2.1 g, 53 mmol) and the mixture was stirred at room temperature overnight. The mixture was diluted with water (1.0 L), extracted with EtOAc (2×1.5 L) and the combined organic layers washed with brine (2×1.0 L), dried over Na₂SO₄, filtered and concentrated under reduced pressure. The residue was purified by silica gel chromatography (Pet. ether/EtOAc=1/1, v/v) to give 4-(5-cyano-2-methoxyphenyl)-6-methylnicotinic acid (3.9 g, 83%) as an off-white solid. LCMS (ESI, m/z): 269 [M+H]⁺. ¹H NMR (400 MHz, DMSO-d₆) δ 8.82 (s, 1H), 7.88 (dd, J=8.6, 2.2 Hz, 1H), 7.71 (d, J=2.2 Hz, 1H), 7.28-7.20 (m, 2H), 3.75 (s, 3H), 2.54 (s, 3H).

TABLE AI
The following intermediate was prepared using a similar procedure to that
described for Intermediate 2:

| Intermediate | | Compound | |
|---|---|---|---|
| Number | Structure | Name | Characterization Data |
| 56 | 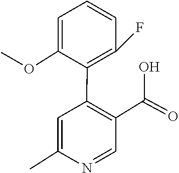 [SMILES: COc1cccc(F)c1-c1cc(C)ncc1C(=O)O] | 4-(2-Fluoro-6- methoxyphenyl)- 6-methylnicotinic acid | LCMS (m/z): 262 [M + H]⁺. ¹H NMR (400 MHz, DMSO-d₆) δ 8.89 (s, 1H), 7.37 (q, J = 8.0 Hz, 1H), 7.18 (s, 1H), 6.92 (d, J = 8.4 Hz, 1H), 6.86 (t, J = 8.8 Hz, 1H), 3.68 (s, 3H), 2.53 (s, 3H) |

### Intermediate 3: 2′-Chloro-5′-methoxy-6-methyl-[4,4′-bipyridine]-3-carboxylic acid

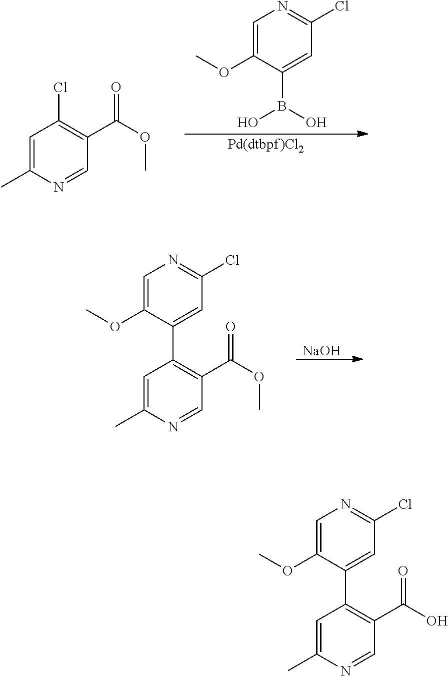

Step 1: To a solution of methyl 4-chloro-6-methylnicotinate (6.00 g, 32.1 mmol) and (2-chloro-5-methoxypyridin-4-yl)boronic acid (15.0 g, 80.2 mmol) in 1,4-dioxane (140 mL) and water (14 mL) was added Pd(dtbpf)Cl₂ (2.1 g, 3.2 mmol) and K₂CO₃ (13.3 g, 96.3 mmol) and the mixture was heated at 80° C. for 2 h. The mixture was diluted with water (200 mL), extracted with EtOAc (3×200 mL), and the combined organic layers were washed with water (200 mL), brine (200 mL), dried over Na₂SO₄, filtered and concentrated under reduced pressure. The residue was purified by silica gel chromatography (EtOAc/Pet. Ether=1/1) to give methyl 2′-chloro-5′-methoxy-6-methyl-[4,4′-bipyridine]-3-carboxylate (7.3 g, 31%) as a yellow solid. LCMS (ESI, m/z): 293 [M+H]⁺, ¹H NMR (400 MHz, Chloroform-d) δ 9.05 (s, 1H), 8.04 (s, 1H), 7.17 (s, 1H), 7.08 (s, 1H), 3.81 (s, 3H), 3.75 (s, 3H), 2.67 (s, 3H).

Step 2: To a solution of methyl 2′-chloro-5′-methoxy-6-methyl-[4,4′-bipyridine]-3-carboxylate (14.6 g, 50.0 mmol) in MeOH (140 mL) and water (140 mL) at 0° C. was added NaOH (6.00 g, 150 mmol) and the mixture was stirred at room temperature for 2 h. The mixture was adjusted to pH 5-6 with 2 M HCl and concentrated under reduced pressure. The residue was purified on a silica gel column (DCM/MeOH=10/1, v/v) to give 2′-chloro-5′-methoxy-6-methyl-[4,4′-bipyridine]-3-carboxylic acid (14.5 g) as a yellow solid. LCMS (ESI, m/z): 279 [M+H]⁺, ¹H NMR (400 MHz, DMSO-d₆) δ 8.75 (s, 1H), 8.09 (s, 1H), 7.16 (s, 1H), 6.95 (s, 1H), 3.74 (s, 3H), 2.45 (s, 3H).

TABLE AJ
The following intermediate was prepared using a similar procedure to that
described for Intermediate 3

| Intermediate | | | |
|---|---|---|---|
| Number | Structure | Compound Name | Characterization Data |
| 57 | 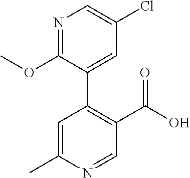 [SMILES: COc1ncc(Cl)cc1-c1cc(C)ncc1C(=O)O] | 5-Chloro-2-methoxy- 6′-methyl-[3,4′- bipyridine]-3′- carboxylic acid | LCMS (m/z): 279 [M + H]⁺. ¹H NMR (400 MHz, DMSO-d₆) δ 8.88 (s, 1H), 8.26 (d, J = 2.6 Hz, 1H), 7.86 (d, J = 2.6 Hz, 1H), 7.40 (s, 1H), 3.77 (s, 3H), 2.57 (s, 3H) |

### Intermediate 4: 4-(6-methoxyimidazo[1,5-a]pyridin-7-yl)-6-methylnicotinic acid

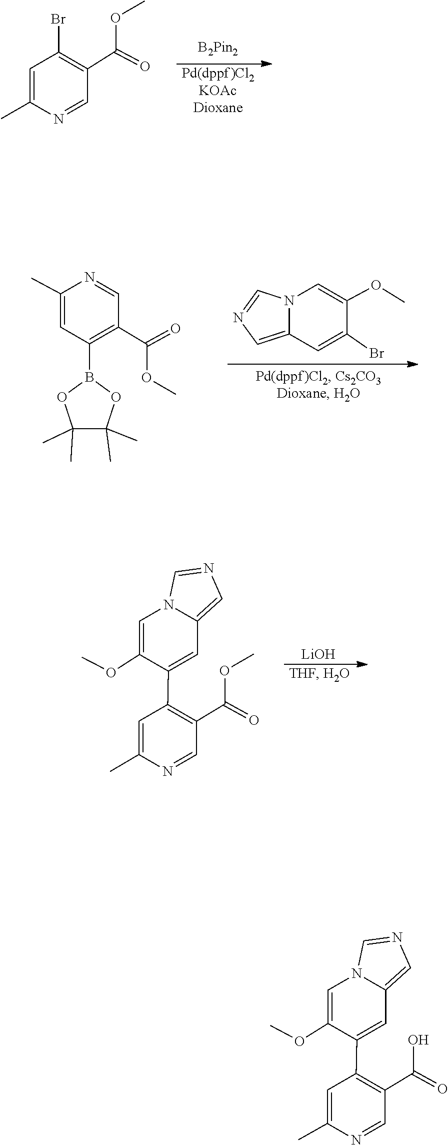

Step 1: A solution of methyl 4-bromo-6-methylpyridine-3-carboxylate (2.0 g, 8.7 mmol) and B₂Pin₂ (2.7 g, 10 mmol) in 1,4-dioxane (30 mL) was added KOAc (2.6 g, 26 mmol) and Pd(dppf)Cl₂ (1.4 g, 1.9 mmol) in one portion at 25° C. The reaction mixture was charged with N2 three times at 25° C. The resulting mixture was heated to 80° C. and stirred for 5 h at 90° C. under a nitrogen atmosphere. The reaction mixture was cooled to 25° C. and concentrated under reduced pressure. The residue was purified by silica gel column chromatography, eluted with Pet. ether/EtOAc (1/1) to afford methyl 6-methyl-4-(4,4,5,5-tetramethyl-1,3,2-dioxaborolan-2-yl)nicotinate (1.5 g, 62% yield) as a light yellow solid. LCMS (ESI): [M+H]=278.

Step 2: Into a 30 mL sealed tube were added methyl 6-methyl-4-(4,4,5,5-tetramethyl-1,3,2-dioxaborolan-2-yl)nicotinate (610 mg, 2.2 mmol), Pd(dppf)Cl₂ (359 mg, 0.4 mmol), 7-bromo-6-methoxyimidazo[1,5-a]pyridine (500 mg, 2.2 mmol), Cs₂CO₃ (2.2 g, 6.6 mmol), 1,4-dioxane (10 mL) and H₂O (1 mL) at 25° C. The reaction mixture was charged with N2 three times at 25° C. The resulting mixture was heated at 90° C. and stirred for 5 hs at 90° C. under a nitrogen atmosphere. The reaction mixture was cooled to 25° C., diluted with water (10 mL) and extracted with EtOAc (3×20 mL). The combined organic layers were washed with brine, dried over anhydrous Na₂SO₄. After filtration, the filtrate was concentrated under reduced pressure. The residue was purified by reverse-phase flash chromatography (0-100% ACN in water with 0.1% FA modifier) to afford methyl 4-{6-methoxyimidazo[1,5-a]pyridin-7-yl}-6-methylpyridine-3-carboxylate (330 mg, 50% yield) as a light yellow solid. LCMS (ESI): [M+H]⁺=298.

Step 3: Into an 8 mL vial were added methyl 4-{6-methoxyimidazo[1,5-a]pyridin-7-yl}-6-methylpyridine-3-carboxylate (300 mg, 1.0 mmol), NaOH (121 mg, 3.0 mmol), MeOH (3 mL) and water (1 mL) at 25° C. The reaction mixture was stirred overnight at 25° C. The resulting mixture was concentrated under reduced pressure. The residue was purified by reverse-phase flash chromatography (0-100% ACN in water with 0.1% FA modifier) to afford 4-{6-methoxyimidazo[1,5-a]pyridin-7-yl}-6-methylpyridine-3-carboxylic acid (290 mg, 89% yield) as a light yellow solid. LCMS (ESI): [M+H]⁺=284.

TABLE AK
The following intermediates was prepared using a similar procedure to that
described for Intermediate 4

| Intermediate | | | LCMS |
|---|---|---|---|
| Number | Structure | Compound Name | [M + H]⁺ |
| 58 | 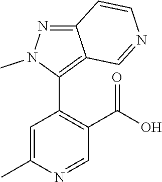 [SMILES: Cc1cc(-c2c3cnccc3nn2C)c(C(=O)O)cn1] | 6-methyl-4-(2-methyl- 2H-pyrazolo[4,3-c]pyridin-3-yl) nicotinic acid | 269 |
| 59 | 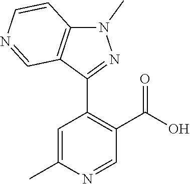 [SMILES: Cc1cc(-c2nn(C)c3ccncc23)c(C(=O)O)cn1] | 6-methyl-4-(1-methyl- 1H-pyrazolo[4,3-c]pyridin-3-yl) nicotinic acid | 269 |

### Intermediate 60: 4-(6-(difluoromethyl)-3-methoxypyridazin-4-yl)-6-methylnicotinic acid

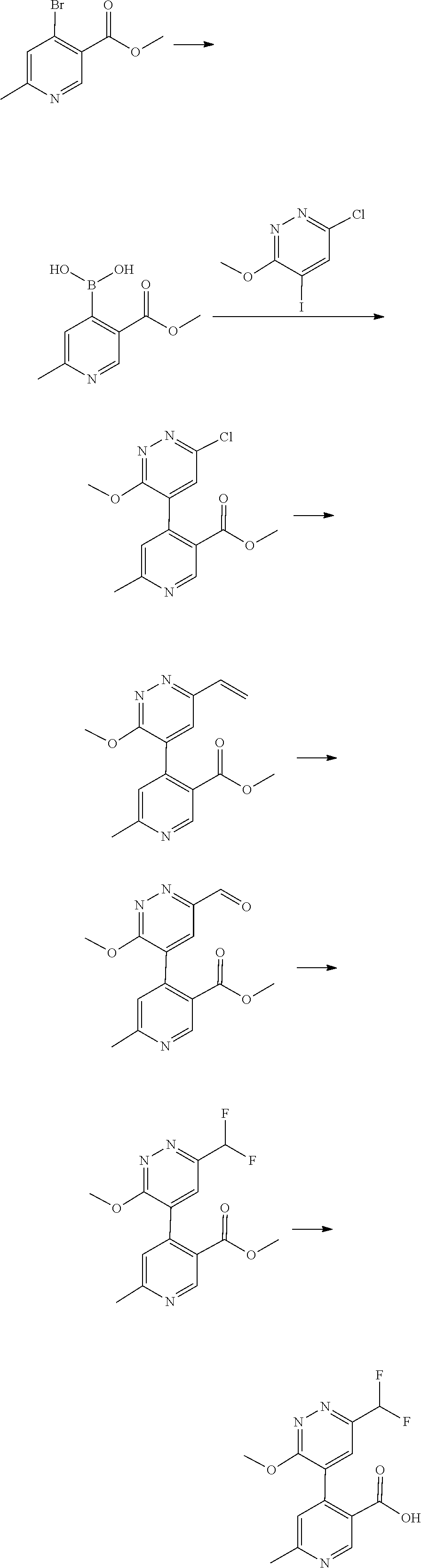

Step 1: To a stirred solution of methyl 4-bromo-6-methylnicotinate (10 g, 43 mmol) in 1,4-dioxane (300 mL) was added bis(pinacolato)diborane (22 g, 87 mmol), Pd(dppf)Cl₂—CH₂Cl₂ (3.5 g, 4.3 mmol), and potassium acetate (13 g, 8.2 mL, 0.13 mol) in one portion at 25° C. under a nitrogen atmosphere. The reaction mixture was stirred at 80° C. for 3 h under a nitrogen atmosphere. The mixture was cooled to room temperature and concentrated under reduced pressure. The residue was purified by silica gel column chromatography, eluting with pet ether:EtOAc (1:1) to afford (5-(methoxycarbonyl)-2-methylpyridin-4-yl)boronic acid (15 g) as a brown oil which was used crude in the next step.

Step 2: To a stirred solution of (5-(methoxycarbonyl)-2-methylpyridin-4-yl)boronic acid (10 g, 0.05 mol) and 6-chloro-4-iodo-3-methoxypyridazine (7 g, 0.03 mol) in 1,4-dioxane (250 mL) and H₂O (25 mL) were added Pd(dppf)Cl₂—CH₂Cl₂ (2 g, 3 mmol) and potassium carbonate (10 g, 5 mL, 0.08 mol) in portions at 25° C. under a nitrogen atmosphere. The resulting mixture was stirred for 2 h at 100° C. under a nitrogen atmosphere. The mixture was cooled to room temperature and extracted with EtOAc (3×100 ml). The combined organic layers were washed with brine (2×20 ml) and dried over anhydrous Na₂SO₄. After filtration, the filtrate was concentrated under reduced pressure. The residue was purified by silica gel column chromatography, eluting with pet ether:EtOAc (1:1) to afford methyl 4-(6-chloro-3-methoxypyridazin-4-yl)-6-methylnicotinate (4.7 g, 60% yield) as yellow solid.

Step 3: To a stirred solution of methyl 4-(6-chloro-3-methoxypyridazin-4-yl)-6-methylnicotinate (6.5 g, 22 mmol) and vinyl boronic acid pinacol ester (6.8 g, 7.5 mL, 44 mmol) in 1,4-dioxane (200 mL) and H₂O (20 mL) were added potassium carbonate (9.2 g, 3.9 mL, 66 mmol) and Pd(dppf)Cl₂—CH₂Cl₂ (1.8 g, 2.2 mmol) in portions at 25° C. under a nitrogen atmosphere. The resulting mixture was stirred for 2 h at 100° C. under a nitrogen atmosphere.

The mixture was cooled to 25° C. and the resulting mixture was concentrated under reduced pressure. The mixture was extracted with EtOAc (3×50 ml) and the combined organic layers were washed with brine (2×20 ml) and dried over anhydrous Na₂SO₄. After filtration, the filtrate was concentrated under reduced pressure. The residue was purified by silica gel column chromatography, eluting with pet ether:EtOAc (1:1) to afford methyl 4-(3-methoxy-6-vinylpyridazin-4-yl)-6-methylnicotinate (4 g, 60% yield) as white solid.

Step 4: To a stirred solution of methyl 4-(3-methoxy-6-vinylpyridazin-4-yl)-6-methylnicotinate (4 g, 0.01 mol) and N-methylmorpholine N-oxide (2 g, 0.01 mol) in DMA (80 mL) were added potassium osmate(VI) dihydrate (0.5 g, 1 mmol) and sodium metaperiodate (10 g, 3 mL, 0.06 mol) in portions at 25° C. under a nitrogen atmosphere. The resulting mixture was stirred for 2 days at 25° C. under a nitrogen atmosphere. The mixture was extracted with EtOAc (3×100 ml) and the combined organic layers were washed with brine (5×30 ml) and dried over anhydrous Na₂SO₄. After filtration, the filtrate was concentrated under reduced pressure. The residue was purified by silica gel column chromatography, eluting with pet ether:EtOAc (1:1) to afford methyl 4-(6-formyl-3-methoxypyridazin-4-yl)-6-methylnicotinate (2 g, 50% yield) as white solid.

Step 5: A solution of methyl 4-(6-formyl-3-methoxypyridazin-4-yl)-6-methylnicotinate (580 mg, 2.02 mmol) in DCM (20 mL) was stirred for 20 min at −78° C. under a nitrogen atmosphere. To the above mixture was added DAST (1.46 g, 1.18 mL, 9.09 mmol) dropwise over 10 min at −78° C. The resulting mixture was stirred for 16 h at −78° C.-25° C. The reaction was quenched with aqueous NaHCO₃ (15 ml) at 0° C. The resulting mixture was extracted with DCM (3×50) and the combined organic layers were washed with brine (2×20 ml) and dried over anhydrous Na₂SO₄. After filtration, the filtrate was concentrated under reduced pressure.

The residue was purified by silica gel column chromatography, eluting with pet ether:EtOAc (1:1) to afford methyl 4-(6-(difluoromethyl)-3-methoxypyridazin-4-yl)-6-methylnicotinate (600 mg, 96% yield) as white solid.

Step 6: To a stirred solution of methyl 4-(6-(difluoromethyl)-3-methoxypyridazin-4-yl)-6-methylnicotinate (800 mg, 2.59 mmol) in THF (9 mL) and H₂O (3 mL) was added LiOH (217 mg, 144 μL 5.17 mmol) in portions at 25° C. The reaction was stirred for 2 h at 25° C.

The resulting mixture was concentrated under reduced pressure to afford 4-(6-(difluoromethyl)-3-methoxypyridazin-4-yl)-6-methylnicotinic acid (600 mg, 79% yield) as white solid. LCMS (ESI): [M+H]⁺=296.

TABLE AL
The following intermediates were prepared using a similar procedure to that
described for Intermediate 60.

| Intermediate | | | LCMS |
|---|---|---|---|
| Number | Structure | Compound Name | [M + H]⁺ |
| 61 | 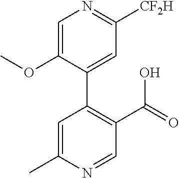 [SMILES: COc1cnc(C(F)F)cc1-c1cc(C)ncc1C(=O)O] | 2′-(difluoromethyl)-5′- methoxy-6-methyl-[4,4′- bipyridine]-3-carboxylic acid | 295 |

### Intermediate 62: 4-(6-methoxy-4-methyl-3,4-dihydro-2H-pyrido[3,2-b][1,4]oxazin-7-yl)-6-methylnicotinic acid

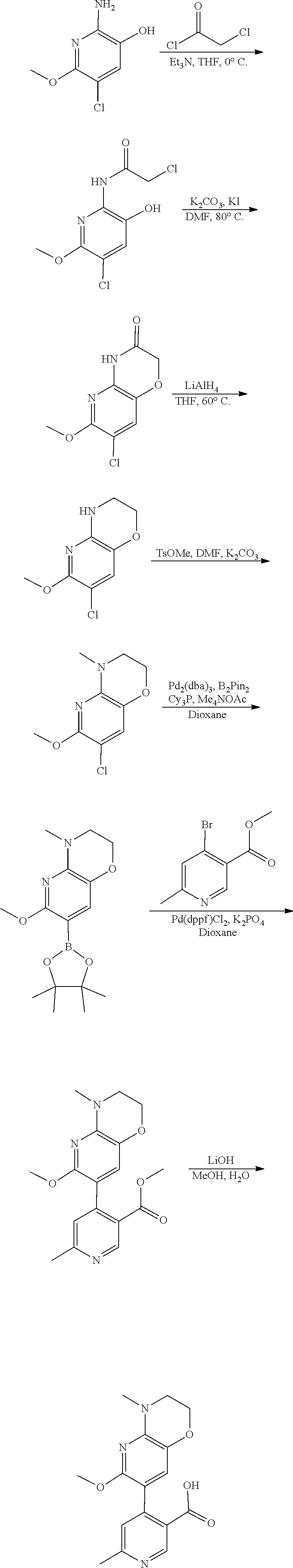

Step 1: To a stirred solution of 2-amino-5-chloro-6-methoxypyridin-3-ol (1.9 g, 11 mmol) and TEA (1.4 g, 1.9 mL, 14 mmol) in THF (30 mL) was added 2-chloroacetyl chloride (1.4 g, 12 mmol) dropwise at 0° C. under a nitrogen atmosphere. The resulting mixture was stirred at 25° C. for 2 h under a nitrogen atmosphere. The reaction was quenched with water (30 mL) at 0° C. The resulting mixture was extracted with EtOAc (3×30 mL) and dried over anhydrous Na₂SO₄. After filtration, the filtrate was concentrated under reduced pressure to afford 2-chloro-N-(5-chloro-3-hydroxy-6-methoxypyridin-2-yl)acetamide (2.2 g, crude) as a brown solid. LCMS (ESI, m/z): 251 [M+H]⁺.

Step 2: To a stirred solution of 2-chloro-N-(5-chloro-3-hydroxy-6-methoxypyridin-2-yl)acetamide (2.1 g, 8.4 mmol) and K₂CO₃ (1.4 g, 10.1 mmol) in DMF (20 mL) was added KI (97 mg, 583 μmol) at 25° C. The resulting mixture was stirred at 25° C. for 2 h. The reaction was quenched with water (30 mL) at 0° C. The resulting mixture was extracted with EtOAc (3×30 mL). The combined organic layers were washed with brine and dried over anhydrous Na₂SO₄. After filtration, the filtrate was concentrated under reduced pressure to afford 7-chloro-6-methoxy-2H-pyrido[3,2-b][1,4]oxazin-3(4H)-one (1.4 g, crude) as a grey solid. LCMS (ESI, m/z): 215 [M+H]⁺.

Step 3: To a stirred solution of 7-chloro-6-methoxy-2H-pyrido[3,2-b][1,4]oxazin-3(4H)-one (1.4 g, 6.5 mmol) in THF (8 mL) was added LiAlH₄ (0.3 g, 7.8 mmol) in portions at 0° C. for 20 minutes. The reaction mixture was stirred at 60° C. for 3 h. The resulting mixture was cooled to 25° C., and quenched with water (0.3 mL), NaOH (15%, 0.3 mL) and water (0.3 mL) in sequence at 0° C. After filtration, the filter cake was washed with EtOAc (3×50 mL). The filtrate was dried over anhydrous Na₂SO₄, filtered, and concentrated under reduced pressure to afford 7-chloro-6-methoxy-3,4-dihydro-2H-pyrido[3,2-b][1,4]oxazine (1.4 g, crude) as a brown oil which used directly in next step without further purification. LCMS (ESI, m/z): 201 [M+H]⁺.

Step 4: To a solution of 7-chloro-6-methoxy-3,4-dihydro-2H-pyrido[3,2-b][1,4]oxazine (1.4 g, 6.7 mmol) and methyl toluene-4-sulfonate (1.9 g, 10 mmol) in DMF (10 mL) was added K₂CO₃ (2.8 g, 20.2 mmol) in one portion at 25° C. The reaction mixture was stirred at 60° C. for 2 h. The resulting mixture was cooled to 25° C. then quenched with water (100 mL). The mixture was extracted with EtOAc (3×50 mL) and the combined organic layers were washed with brine and dried over anhydrous Na₂SO₄. After filtration, the filtrate was concentrated under reduced pressure. The residue was purified by reverse-phase flash chromatography (55-63% MeCN in water with 0.10% formic acid modifier) to afford 7-chloro-6-methoxy-4-methyl-3,4-dihydro-2H-pyrido[3,2-b][1,4]oxazine (770 mg, 53% yield) as a grey solid. LCMS (ESI, m/z): 215 [M+H]⁺.

Step 5: To a stirred solution of 7-chloro-6-methoxy-4-methyl-3,4-dihydro-2H-pyrido[3,2-b][1,4]oxazine (500 mg, 2.3 mmol) and bis(pinacolato)diboron (887 mg, 3.5 mmol) in 1,4-dioxane (10 mL) were added tricyclohexylphosphane (131 mg, 466.0 μmol), tetramethylammonium acetate (931 mg, 7.0 mmol), and Pd₂(dba)₃ (213 mg, 233.0 μmol) in sequence at 25° C. The reaction mixture was charged with nitrogen three times, and the resulting mixture was stirred at 100° C. for 2 h under a nitrogen atmosphere. The reaction mixture was cooled to 25° C. and diluted with water (50 mL). The resulting mixture was extracted with EtOAc (3×50 mL), and the combined organic layers were washed with brine and dried over anhydrous Na₂SO₄. After filtration, the filtrate was concentrated under reduced pressure. The residue was purified by silica gel column chromatography, eluting with pet ether:EtOAc (5/1) to afford 6-methoxy-4-methyl-7-(4,4,5,5-tetramethyl-1,3,2-dioxaborolan-2-yl)-3,4-dihydro-2H-pyrido[3,2-b][1,4]oxazine (600 mg, 84% yield) as a yellow solid. LCMS (ESI, m/z): 307 [M+H]⁺.

Step 6: To a stirred mixture of 6-methoxy-4-methyl-7-(4,4,5,5-tetramethyl-1,3,2-dioxaborolan-2-yl)-3,4-dihydro-2H-pyrido[3,2-b][1,4]oxazine (600 mg, 2.0 mmol) and methyl 4-bromo-6-methylnicotinate (451 mg, 2.0 mmol) in 1,4-dioxane (20 mL) was added Pd(dppf)Cl₂·CH₂Cl₂ (160 mg, 196 μmol) and K₃PO₄ (1.3 g, 5.9 mmol) in sequence at 25° C.

The reaction mixture was charged with nitrogen three times and stirred at 100° C. for 2 h under a nitrogen atmosphere. The reaction mixture was cooled to 25° C. and quenched with water (50 mL). The resulting mixture was extracted with EtOAc (3×50 mL). The combined organic layers were washed with brine and dried over anhydrous Na₂SO₄. After filtration, the filtrate was concentrated under reduced pressure. The residue was purified by silica gel column chromatography, eluting with pet ether:EtOAc (1/1) to afford methyl 4-(6-methoxy-4-methyl-3,4-dihydro-2H-pyrido[3,2-b][1,4]oxazin-7-yl)-6-methylnicotinate (500 mg, 56% yield) as a yellow solid. LCMS (ESI, m/z): 330 [M+H]⁺.

Step 7: A mixture of methyl 4-(6-methoxy-4-methyl-3,4-dihydro-2H-pyrido[3,2-b][1,4]oxazin-7-yl)-6-methylnicotinate (400 mg, 1.2 mmol) and LiOH (86 mg, 3.6 mmol) in MeOH (6 mL) and H₂O (2 mL) was stirred at 25° C. for 2 h. The resulting mixture was acidified to pH 4 with HCl (1N) and concentrated under reduced pressure. The residue was purified by reverse-phase flash chromatography (10-50% ACN in water with 0.1% formic acid modifier) to afford 4-(6-methoxy-4-methyl-3,4-dihydro-2H-pyrido[3,2-b][1,4]oxazin-7-yl)-6-methylnicotinic acid (320 mg, 84% yield) as a yellow solid. LCMS (ESI, m/z): 316 [M+H].

### Intermediate 63: 2′-chloro-6-cyclopropyl-5′-methoxy-[4,4′-bipyridine]-3-carboxylic acid

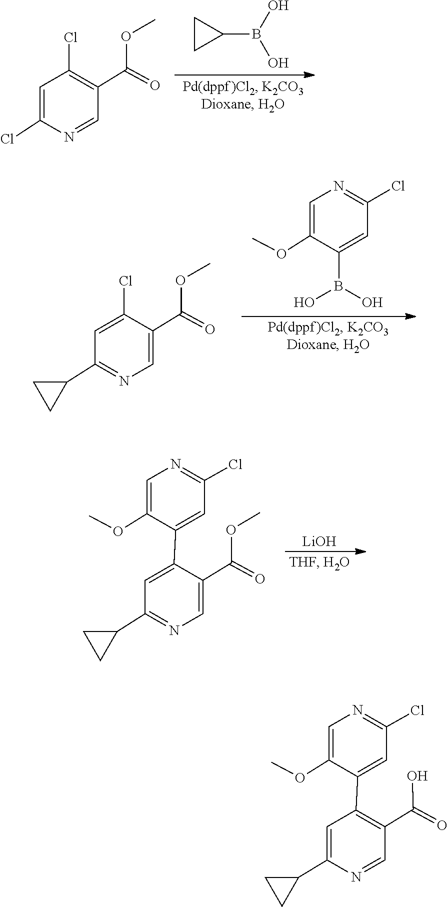

Step 1: To a solution of methyl 4,6-dichloronicotinate (15 g, 73 mmol) in 1,4-dioxane (120 mL) and water (12 mL) was added cyclopropylboronic acid (7.5 g, 87 mmol), Pd(dppf)Cl₂ (2.7 g, 3.6 mmol), and K₂CO₃ (20 g, 0.2 mmol) in one portion at 25° C. The reaction mixture was charged with N2 three times, and the mixture was stirred at 100° C. for 16 h under a nitrogen atmosphere. The resulting mixture was cooled to 25° C. and concentrated under reduced pressure. The residue was purified by silica gel column chromatography, eluting with pet ether:EtOAc (3/1) to afford methyl 4-chloro-6-cyclopropylnicotinate (5.6 g, 36% yield) as a white oil. LCMS (ESI, m/z): 212 [M+H]⁺.

Step 2: To a solution of methyl 4-chloro-6-cyclopropylnicotinate (5.6 g, 26 mmol) in 1,4-dioxane (100 mL) and water (10 mL) was added (2-chloro-5-methoxypyridin-4-yl)boronic acid (5.9 g, 32 mmol), Pd(dppf)Cl₂ (1.0 g, 1.3 mmol), and K₂CO₃ (11 g, 79 mmol) in one portion at 25° C. The reaction mixture was charged with N2 and stirred at 120° C. for 16 h under a nitrogen atmosphere. The resulting mixture was cooled to 25° C. and concentrated under reduced pressure. The residue was purified by silica gel column chromatography, eluting with pet ether:EtOAc (2/1) to afford methyl 2′-chloro-6-cyclopropyl-5′-methoxy-[4,4′-bipyridine]-3-carboxylate (1.6 g, 20% yield) as a white solid. LCMS (ESI, m/z): 319 [M+H]⁺.

Step 3: To a solution of methyl 2′-chloro-6-cyclopropyl-5′-methoxy-[4,4′-bipyridine]-3-carboxylate (1.6 g, 5.0 mmol) in THF (12 mL) and water (4 mL) was added lithium hydroxide (360 mg, 15.0 mmol) in one portion at 25° C. The reaction mixture was stirred at 80° C. for 2 h. The resulting mixture was acidified to pH 6 with HCl (1N) and concentrated under reduced pressure. MeOH (10 mL) was added, and the mixture was filtered. The filter cake was washed with MeOH (3×20 mL), and the filtrate was concentrated under reduced pressure. The residue was purified by reverse-phase flash chromatography (10-50% ACN in water with 0.1% formic acid modifier) to afford 2′-chloro-6-cyclopropyl-5′-methoxy-[4,4′-bipyridine]-3-carboxylic acid (880 mg, 58% yield) as a white solid. LCMS (ESI, m/z): 305 [M+H]⁺.

### Intermediate 64: 4-(6-methoxy-1-methyl-1H-indazol-5-yl)-6-methylnicotinic acid

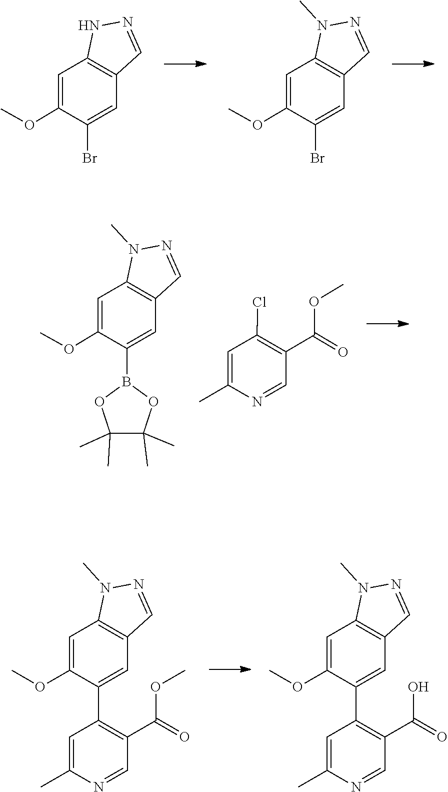

Step 1: To a solution of 5-bromo-6-methoxy-1H-indazole (2.00 g, 8.85 mmol) in DMF (20 mL) at 0° C. was added NaH (60% w/w in oil, 708 mg, 17.7 mmol) and Mel (1.9 g 13 mmol) and the mixture was stirred at room temperature for 2 h. The mixture was diluted with water (50 mL), extracted with EtOAc (2×50 mL) and the combined organic layers washed with brine (50 mL), dried over Na₂SO₄, filtered and concentrated under reduced pressure. The residue was purified by silica gel chromatography (Petroleum Ether/EtOAc=5/1, v/v) to afford 5-bromo-6-methoxy-1-methyl-TH-indazole (1.2 g, 57% yield) as a white solid. ¹H NMR (400 MHz, Chloroform-d) δ 7.89 (s, 1H), 7.83 (s, 1H), 6.74 (s, 1H), 4.03 (s, 3H), 3.98 (s, 3H).

Step 2: To a solution of 5-bromo-6-methoxy-1-methyl-1H-indazole (500 mg, 2.1 mmol) in 1-4 dioxane (8 mL) was added Pin₂B₂ (1.33 g, 5.25 mmol), Pd(dppf)Cl₂ (152.3 mg, 0.21 mmol) and KOAc (617.4 mg, 6.3 mmol) and the mixture was heated at 100° C. overnight under N2. The mixture was filtered and the filtrate concentrated under reduced pressure to afford 6-methoxy-1-methyl-5-(4,4,5,5-tetramethyl-1,3,2-dioxaborolan-2-yl)-1H-indazole (600 mg, 99% yield), which was used directly in the next step. LCMS (ESI, m/z): 289 [M+H]⁺.

Step 3: To a solution of 6-methoxy-1-methyl-5-(4,4,5,5-tetramethyl-1,3,2-dioxaborolan-2-yl)-1H-indazole (600 mg, 2.08 mmol) in 1,4-dioxane (8 mL) and water (2 mL) was added methyl 4-chloro-6-methylnicotinate (772 mg, 4.17 mmol), Pd(dtbpf)Cl₂ (134 mg, 0.208 mmol) and K₂CO₃ (861 mg, 6.24 mmol) and the mixture was heated at 100° C. overnight under N2. The mixture was diluted with water (50 mL), extracted with EtOAc (2×50 mL) and the combined organic layers washed with brine (50 mL), dried over Na₂SO₄, filtered and concentrated under reduced pressure. The residue was purified by silica gel chromatography (Petroleum Ether/EtOAc=1/1) to afford methyl 4-(6-methoxy-1-methyl-1H-indazol-5-yl)-6-methylnicotinate (450 mg, 69% yield) as a yellow solid. LCMS (ESI, m/z): 312 [M+H]⁺.

Step 4: To a solution of methyl 4-(6-methoxy-1-methyl-1H-indazol-5-yl)-6-methylnicotinate (50 mg, 0.16 mmol) in MeOH (2 mL) and water (2 mL) was added NaOH (20 mg, 0.48 mmol) and the mixture was heated at 50° C. for 3 h. The mixture was adjusted to pH 5-6 with 2 M aqueous HCl and the resulting precipitate was collected by filtration to afford 4-(6-methoxy-1-methyl-1H-indazol-5-yl)-6-methylnicotinic acid (40 mg, 84% yield) as a yellow solid. LCMS (ESI, m/z): 298 [M+H].

### Intermediate 65 & 66: 3-bromo-2-methyl-2H-pyrazolo[4,3-c]pyridine (65) & 3-bromo-1-methyl-1H-pyrazolo[4,3-c]pyridine (66)

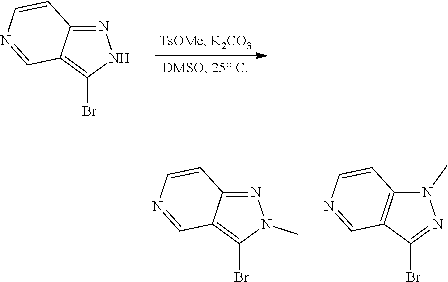

Into a 25 mL 3-necked round-bottom flask were added 3-bromo-2H-pyrazolo[4,3-c]pyridine (10 g, 50 mmol), K₂CO₃ (10 g, 76 mmol) and methyl 4-methylbenzenesulfonate (9.4 g, 50 mmol) in DMSO (60 mL) at 0° C. The resulting mixture was stirred at 25° C. for 5 h. The reaction was quenched with water (700 mL) at 0° C. The resulting mixture was extracted with DCM/MeOH (20/1, 3×500 mL). The combined organic layers were washed with brine (3×500 mL) and dried over anhydrous Na₂SO₄. After filtration, the filtrate was concentrated under reduced pressure. The residue was purified by prep-HPLC (30-45% ACN in water with 10 mmol/L NH₄HCO₃ modifier to afford two compounds:

3-bromo-2-methyl-2H-pyrazolo[4,3-c]pyridine (600 mg, 5.6% yield) as a white solid LCMS (ESI, m/z): 212 and 214 [M+H]⁺.

3-bromo-1-methyl-1H-pyrazolo[4,3-c]pyridine (4.9 g, 46% yield) as a white solid. LCMS (ESI, m/z): 212 and 214 [M+H]⁺.

### Intermediate 5: 6-Bromothiazolo[4,5-b]pyridin-2-amine

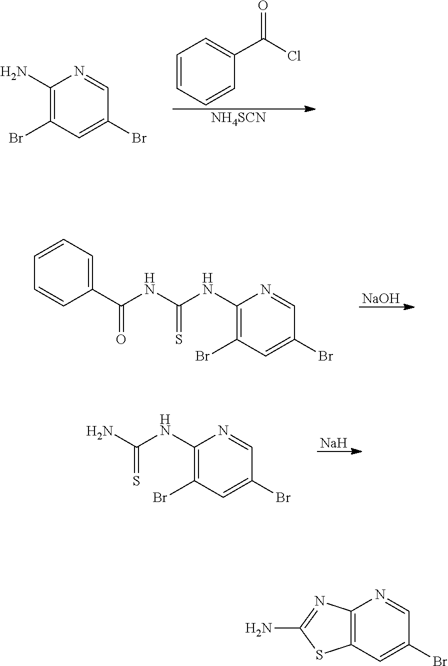

Step 1: To a solution of 3,5-dibromopyridin-2-amine (100 g, 397 mmol) in THF (1 L) was added ammonium thiocyanate (51 g, 675 mmol) and benzoyl chloride (78 g, 556 mmol) and the mixture was heated at reflux for 16 h. After cooling to room temperature, the mixture was concentrated under reduced pressure to give N-((3,5-dibromopyridin-2-yl)carbamothioyl)benzamide, which was used directly in the next step. LCMS (ESI, m/z): 414, 416 [M+H]+

Step 2: To a solution of N-((3,5-dibromopyridin-2-yl)carbamothioyl)benzamide (165 g, 398 mmol) in MeOH (320 mL) and water (400 mL) at 0° C. was added NaOH (121 g, 3.02 mmol) and the mixture was heated at reflux for 3 h. After cooling to room temperature, the mixture was concentrated under reduced pressure to remove MeOH. The residue was poured into water and the resulting precipitate collected by filtration and dried to afford 1-(3,5-dibromopyridin-2-yl)thiourea (90 g, 73%) as a green solid. ¹H NMR (400 MHz, DMSO-d₆) δ 9.69 (s, 1H), 9.32 (s, 1H), 8.61 (s, 1H), 8.51 (s, 1H), 8.43 (s, 1H).

Step 3: To a mixture of NaH (60%, 15.4 g, 386 mmol) in DMF (640 mL) at 0° C. was added 1-(3,5-dibromopyridin-2-yl)thiourea (40 g, 129 mmol) and the mixture was stirred at 0° C. for 15 min, then room temperature for 15 mins then heated at 80° C. for 3 h. After cooling to room temperature, the reaction was quenched with ice cold saturated aqueous NH₄Cl solution (500 mL) and extracted with EtOAc (500 mL×3). The combined organic phases were dried over Na₂SO₄ and concentrated under reduced pressure. The residue was purified by silica gel chromatography (Pet.Ether/EtOAc=10/1-1/1 then DCM/MeOH=50/1-20/1) to give 6-bromothiazolo[4,5-b]pyridin-2-amine (16.4 g, 54%) as a light gray solid. LCMS (ESI, m/z): 230, 232 [M+H]⁺, ¹H NMR (400 MHz, DMSO-d₆) δ 8.31 (d, J=2.2 Hz, 1H), 8.27 (d, J=2.2 Hz, 1H), 8.09 (s, 2H).

### Intermediate 6: 6-(4-Chlorophenyl)thiazolo[4,5-b]pyridin-2-amine

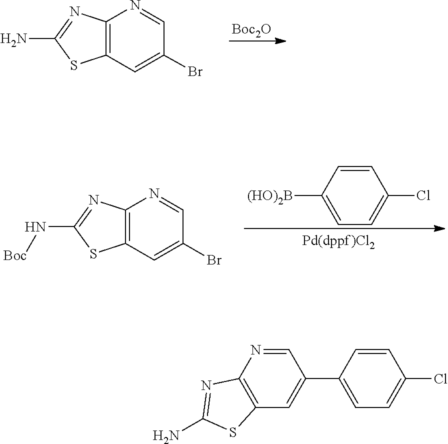 [SMILES: "Boc2O" ; Nc1nc2ncc(Br)cc2s1 ; CC(C)(C)OC(=O)Nc1nc2ncc(Br)cc2s1 ; OB(O)c1ccc(Cl)cc1 ; Nc1nc2ncc(-c3ccc(Cl)cc3)cc2s1]

Step 1: To a solution of 6-bromothiazolo[4,5-b]pyridin-2-amine (10 g, 44 mmol) in DMF (2 mL) was added (Boc)₂O (19 g, 87 mmol), TEA (13.2 g, 130 mmol) and DMAP (1.1 g, 8.7 mmol) and the mixture was stirred at room temperature for 2 h. The mixture was diluted with water (200 mL), extracted with EtOAc (3×300 mL) and the combined organic layers washed with water (200 mL), brine (200 mL), dried over Na₂SO₄, filtered and concentrated under reduced pressure. The residue was purified by silica gel chromatography (Pet. Ether/EtOAc=100/0-3/1, v/v) to give tert-butyl (6-bromothiazolo[4,5-b]pyridin-2-yl)carbamate (9 g, 65%) as a yellow solid. LCMS (ESI, m/z): 330, 332 [M+H]⁺, ¹H NMR (400 MHz, DMSO-d₆) a 12.2 (s, 1H), 8.68 (d, J=2.2 Hz, 1H), 8.56 (d, J=2.4 Hz, 1H), 1.52 (s, 9H).

Step 2: To a solution of tert-butyl (6-bromothiazolo[4,5-b]pyridin-2-yl)carbamate (2 g, 6 mmol) in 1,4-dioxane (40 mL) and water (8 mL) was added (4-chlorophenyl) boronic acid (2.8 g, 18 mmol), Pd(dppf)Cl₂ (440 mg, 0.6 mmol) and Na₂CO₃ (1.9 g, 18 mmol) and the mixture was heated at 100° C. overnight. The mixture was diluted with water (30 mL), extracted with EtOAc (50 mL×3) and the combined organic layers washed with brine, dried over Na₂SO₄, filtered and concentrated under reduced pressure. The residue was purified by silica gel chromatography (Pet. ether: EtOAc=5/1-1/1 then DCM, then DCM/MeOH=100/1-20/1, v/v) to afford 6-(4-chlorophenyl)thiazolo[4,5-b]pyridin-2-amine (2.3 g, 72%) as a brown solid. LCMS (ESI, m/z): 262, 264 [M+H]⁺, ¹H NMR (400 MHz, DMSO-d₆) δ 8.53 (d, J=2.4 Hz, 1H), 8.39 (d, J=2.2 Hz, 1H), 8.04 (s, 2H), 7.74-7.68 (m, 2H), 7.54-7.50 (m, 2H).

TABLE A
The following intermediates were prepared using a similar procedure to that described
for Intermediate 6

| Intermediate | | Compound | |
|---|---|---|---|
| Number | Structure | Name | Characterization Data |
| 7 | 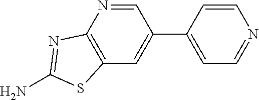 [SMILES: Nc1nc2ncc(-c3ccncc3)cc2s1] | 6-(Pyridin-4- yl)thiazolo[4,5- b]pyridin-2- amine | LCMS (m/z): 229 [M + H]⁺. ¹H NMR (400 MHz, DMSO-d₆) δ 8.69 (d, J = 2.4 Hz, 1H), 8.64- 8.61 (m, 2H), 8.54 (d, J = 2.4 Hz, 1H), 8.15 (s, 2H), 7.76-7.73 (m, 2H) |
| 8 | 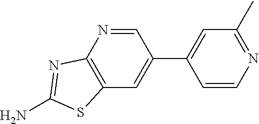 [SMILES: Cc1cc(-c2cnc3nc(N)sc3c2)ccn1] | 6-(2- Methylpyridin- 4- yl)thiazolo[4,5- b]pyridin-2- amine | LCMS (m/z): 243 [M + H]⁺. ¹H NMR (400 MHz, DMSO-d₆) δ 8.66 (d, J = 2.2 Hz, 1H), 8.51 (d, J = 2.2 Hz, 1H), 8.48 (d, J = 5.2 Hz, 1H), 8.13 (s, 2H), 7.65-7.60 (m, 1H), 7.53 (dd, J = 5.2, 1.8 Hz, 1H), 2.52 (s, 3H) |

### Intermediate 9: 6-phenylthiazolo[5,4-c]pyridin-2-amine

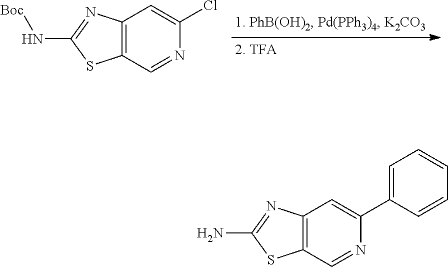 [SMILES: CC(C)(C)OC(=O)Nc1nc2cc(Cl)ncc2s1 ; Nc1nc2cc(-c3ccccc3)ncc2s1]

Step 1: A mixture of tert-butyl (6-chlorothiazolo[5,4-c]pyridin-2-yl)carbamate (200 mg, 0.7 mmol), phenylboronic acid (102 mg, 0.8 mmol), potassium carbonate (290 mg, 2.1 mmol) and Pd(PPh₃)₄ (81 mg, 0.1 mmol, 0.1 equiv.) in 1,4-dioxane (5 mL) and H₂O (0.5 mL) was stirred at 90° C. for 16 h under a nitrogen atmosphere. The resulting mixture was cooled to 25° C. and concentrated under reduced pressure. The residue was purified by reverse-phase flash chromatography (10-50% ACN in water with 0.1% FA modifier) to afford tert-butyl (6-phenylthiazolo[5,4-c]pyridin-2-yl)carbamate (180 mg, 56% yield) as a yellow solid. LCMS (ESI, m/z): [M+H]⁺=328

Step 2: To a stirred solution of tert-butyl (6-phenylthiazolo[5,4-c]pyridin-2-yl)carbamate (340 mg, 1.0 mmol) in DCM (6 mL) was added TFA (0.6 mL) dropwise at 0° C. under a nitrogen atmosphere. The reaction mixture was allowed to warm to 25° C. and stirred at 25° C. for 2 h.

The resulting mixture was concentrated under reduced pressure. The residue was purified by reverse-phase flash chromatography (0-40% ACN in water with 0.1% FA modifier) to afford 6-phenylthiazolo[5,4-c]pyridin-2-amine (70.1 mg, 30% yield) as a yellow solid. LCMS (ESI, m/z): [M+H]⁺=228.

TABLE B
The following intermediates were prepared using a similar procedure to that described
for Intermediate 9

| Intermediate | | | LCMS: |
|---|---|---|---|
| Number | Structure | Compound Name | [M + H]⁺ |
| 10 | 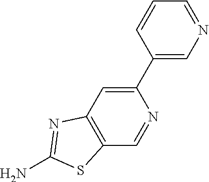 [SMILES: Nc1nc2cc(-c3cccnc3)ncc2s1] | 6-(pyridin-3-yl)thiazolo[5,4- c]pyridin-2-amine | 229 |

### Intermediate 11: 6-Bromothiazolo[4,5-b]pyrazin-2-amine

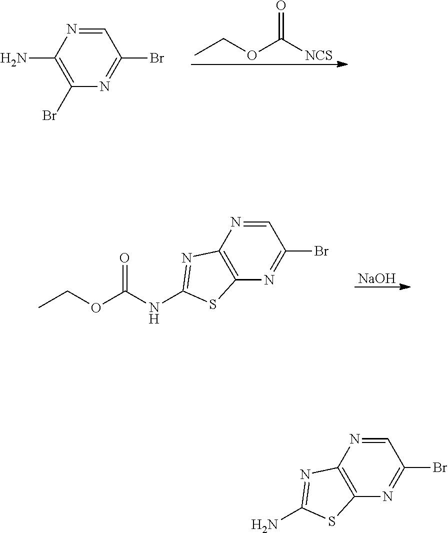

Step 1: To a solution of 3,5-dibromopyrazin-2-amine (20 g, 79 mmol) in acetone (280 mL) was added O-ethyl carbonisothiocyanatidate (104 g, 791 mmol) and the mixture was heated at 100° C. for 1 h in a sealed vessel. After cooling to room temperature, MeOH (50 mL) was added and heating was continued at 80° C. for 1 h in a sealed vessel. The mixture was cooled to 0° C. and stirred for 0.5 h. The resulting precipitate was collected by filtration, washed with 2/1 H₂O/MeOH=(60 mL) then MeOH (20 mL) and dried under reduced pressure to give ethyl (6-bromothiazolo[4,5-b]pyrazin-2-yl)carbamate (18 g, 75%) as light yellow solid. LCMS (ESI, m/z): 303,305 [M+H]⁺, ¹H NMR (400 MHz, DMSO-d₆) δ 12.7 (s, 1H), 8.71 (s, 1H), 4.33-4.25 (m, 2H), 1.32-1.27 (m, 3H).

Step 2: A mixture of ethyl (6-bromothiazolo[4,5-b]pyrazin-2-yl)carbamate (10 g, 33 mmol) and 2 M aqueous NaOH (115 mL) in MeOH (115 mL) was heated at reflux for 5 h. After cooling to room temperature, the mixture was adjusted to pH 5-6 with 2 M aqueous HCl, neutralized with saturated aqueous NaHCO₃ solution and extracted with EtOAc (3×200 mL). The combined organic phases were dried over Na₂SO₄ and concentrated under reduced pressure. The residue was triturated with Pet. ether/EtOAc=10/1 (40 mL) to give 6-bromothiazolo[4,5-b]pyrazin-2-amine (6.5 g, 85%) as a yellow solid. LCMS (ESI, m/z): 231, 233 [M+H], ¹H NMR (400 MHz, DMSO-d₆) δ 8.61 (s, 2H), 8.36 (s, 1H).

### Intermediate 12: 6-(tetrahydrofuran-3-yl)thiazolo[4,5-b]pyrazin-2-amine

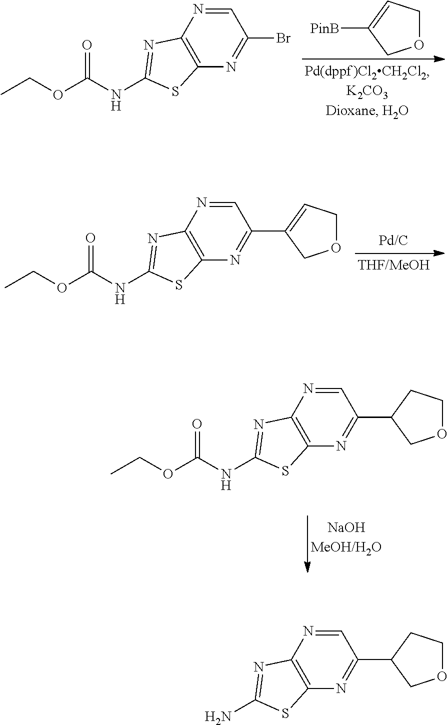

Step 1: To a solution of ethyl N-{6-bromo-[1,3]thiazolo[4,5-b]pyrazin-2-yl}carbamate (1.4 g, 4.6 mmol) and 2-(2,5-dihydrofuran-3-yl)-4,4,5,5-tetramethyl-1,3,2-dioxaborolane (1.0 g, 5.1 mmol) in 1,4-dioxane (50 mL) and H₂O (10 mL) were added K₂CO₃ (1.9 g, 14 mmol) and Pd(dppf)Cl₂·DCM (0.4 g, 0.5 mmol) at 25° C. The reaction mixture was charged with N2 three times at 25° C. The reaction mixture was heated to 110° C. and stirred at 110° C. for 2 h under a nitrogen atmosphere. The reaction mixture was cooled to 25° C. and concentrated under reduced pressure to remove dioxane. The residue was acidified to pH 4 with 1M aq HCl. The mixture was extracted with DCM/MeOH (5/1, 3×100 mL). The combined organic layers were washed with brine (30 mL), dried over anhydrous Na₂SO₄. After filtration, the filtrate was concentrated under reduced pressure. The residue was purified by silica gel column chromatography, eluting with DCM/MeOH (30/1) to afford ethyl N-[6-(2,5-dihydrofuran-3-yl)-[1,3]thiazolo[4,5-b]pyrazin-2-yl]carbamate (1.2 g, 89% yield) as a brown solid. LCMS (ESI, m/z): [M+H]⁺=293

Step 2: To a suspension of ethyl N-[6-(2,5-dihydrofuran-3-yl)-[1,3]thiazolo[4,5-b]pyrazin-2-yl]carbamate (1.0 g, 3.4 mmol) in MeOH (100 mL) and THF (100 mL) was added Pd/C (10%, 300 mg) at 25° C. in a pressure tank. The resulting mixture was heated to 60° C. and stirred at 60° C. under hydrogen (30 psi) for 12 h. The reaction mixture was cooled to 25° C.

After filtration, the filtrate was concentrated under reduced pressure. The residue was purified by silica gel column chromatography, eluting with DCM/MeOH (20/1) to afford ethyl N-[6-(oxolan-3-yl)-[1,3]thiazolo[4,5-b]pyrazin-2-yl]carbamate (700 mg, 70% yield) as a yellow solid. LCMS (ESI, m/z): [M+H]⁺=295.

Step 3: To a suspension of ethyl N-[6-(oxolan-3-yl)-[1,3]thiazolo[4,5-b]pyrazin-2-yl]carbamate (700 mg, 2.4 mmol) in MeOH (10 mL) and H₂O (10 mL) was added NaOH (950 mg, 24 mmol) at 25° C. The reaction mixture was heated at 90° C. for 4 h. The reaction mixture was cooled to 25° C. and concentrated under reduced pressure. The residue was acidified to pH 7 with 1M aq HCl and the mixture was extracted with DCM/MeOH (10/1) (3×100 mL). The combined organic layers were washed with brine (20 mL), dried over anhydrous Na₂SO₄. After filtration, the filtrate was concentrated under reduced pressure. The residue was purified by silica gel column chromatography, eluting with DCM/MeOH (10/1) to afford 6-(oxolan-3-yl)-[1,3]thiazolo[4,5-b]pyrazin-2-amine (400 mg, 76% yield) as a yellow solid. LCMS (ESI, m/z): [M+H]⁺=223.

### Intermediate 13: 6-(4-Chlorophenyl)thiazolo[4,5-b]pyrazin-2-amine

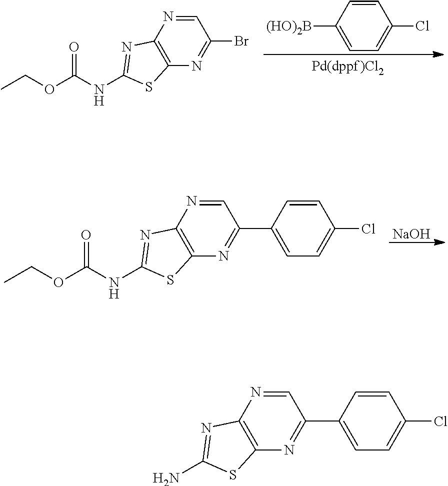

Step 1: To a mixture of ethyl (6-bromothiazolo[4,5-b]pyrazin-2-yl)carbamate (1 g, 3.3 mmol) in 1,4-dioxane (10 mL) and water (2 mL) was added (4-chlorophenyl)boronic acid (1.55 g, 9.90 mmol), Na₂CO₃ (1.05 g, 9.90 mmol) and Pd(dppf)Cl₂ (240 mg, 0.33 mmol) and the mixture was heated at 120° C. for 16 h in sealed tube. After cooling to room temperature, the mixture was diluted with water (5 mL), extracted with EtOAc (3×20 mL) and the combined organic phases dried over Na₂SO₄, filtered and concentrated under reduced pressure. The residue was triturated with Pet. ether/EtOAc=10/1 (10 mL) to give ethyl (6-(4-chlorophenyl)thiazolo[4,5-b]pyrazin-2-yl)carbamate (1.9 g, 86%) as gray solid. LCMS (ESI, m/z): 335 [M+H]⁺.

Step 2: A mixture of ethyl (6-(4-chlorophenyl)thiazolo[4,5-b]pyrazin-2-yl)carbamate (1.9 g, 5.7 mmol) and 2 M aqueous NaOH (18 mL) in MeOH (18 mL) was heated at reflux for 3.5 h. After cooling to room temperature, the mixture was adjusted to pH 5-6 with 2 M aqueous HCl, neutralized with saturated aqueous NaHCO₃ solution and extracted with EtOAc (3×30 mL). The combined organic phases were dried over Na₂SO₄, filtered and concentrated under reduced pressure. The residue was triturated with 5/1 Pet. ether/EtOAc (15 mL) to give 6-(4-chlorophenyl)thiazolo[4,5-b]pyrazin-2-amine (1.2 g, 82%) as a gray solid. LCMS (ESI, m/z): 263 [M+H]⁺, ¹H NMR (400 MHz, DMSO-d₆) δ 8.92-8.83 (m, 1H), 8.53-8.47 (m, 2H), 8.10-8.04 (m, 2H), 7.56-7.51 (m, 2H).

TABLE C
The following intermediates were prepared using a similar procedure to that described
for Intermediate 13

| Intermediate | | | |
|---|---|---|---|
| Number | Structure | Compound Name | Characterization Data |
| 14 | 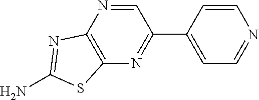 [SMILES: Nc1nc2ncc(-c3ccncc3)nc2s1] | 6-(Pyridin-4- yl)thiazolo[4,5- b]pyrazin-2-amine | LCMS (m/z): 230 [M + H]⁺. ¹H NMR (400 MHz, DMSO-d₆) δ 9.00 (s, 1H), 8.71-8.65 (m, 4H), 8.02 (d, J = 5.2 Hz, 2H) |
| 15 | 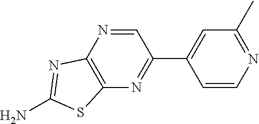 [SMILES: Cc1cc(-c2cnc3nc(N)sc3n2)ccn1] | 6-(2- methylpyridin-4- yl)thiazolo[4,5- b]pyrazin-2-amine | LCMS (m/z): 244 [M + H]⁺. |

### Intermediate 16: 6-(4-Chlorophenyl)thiazolo[4,5-c]pyridin-2-amine

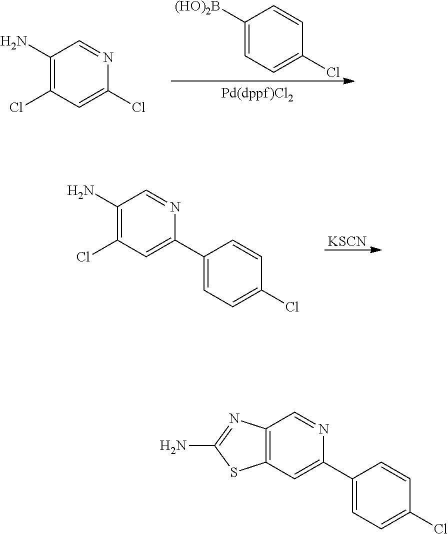 [SMILES: Nc1cnc(Cl)cc1Cl ; OB(O)c1ccc(Cl)cc1 ; Nc1cnc(-c2ccc(Cl)cc2)cc1Cl ; "KSCN" ; Nc1nc2cnc(-c3ccc(Cl)cc3)cc2s1]

Step 1: To a solution of 4,6-dichloropyridin-3-amine (1.0 g, 6.1 mmol) in 1,4-dioxane (10 mL) and water (2 mL) was added (4-chlorophenyl)boronic acid (959 mg, 6.1 mmol), Pd(dppf)Cl₂ (445 mg, 0.61 mmol) and Na₂CO₃ (1.95 g, 18.3 mmol) and the mixture was heated at 80° C. overnight in a sealed tube. The mixture was diluted with water (30 mL), extracted with EtOAc (3×20 mL) and the combined organic layers washed with water (30 mL), brine (30 mL), dried over Na₂SO₄, filtered and concentrated under reduced pressure. The residue was purified by silica gel chromatography (0.6-1% MeOH/DCM) to give 4-chloro-6-(4-chlorophenyl)pyridin-3-amine as a pink solid (1.36 g, 93%). LCMS (ESI, m/z): 239 [M+H]⁺, ¹H NMR (400 MHz, DMSO-d₆): δ 8.17 (s, 1H), 7.98 (d, J=8.6 Hz, 2H), 7.88 (s, 1H), 7.44 (d, J=8.6 Hz, 2H), 5.83 (s, 2H).

Step 2: To a solution of 4-chloro-6-(4-chlorophenyl)pyridin-3-amine (1.36 g, 5.68 mmol) in 1,4-dioxane (10 mL) was added KSCN (2.7 g, 28 mmol) and concentrated aqueous HCl (2 mL) and the mixture was heated at reflux for 2 days. The mixture was diluted with water (200 mL), extracted with EtOAc (3×50 mL) and the combined organic layers washed with water (100 mL), brine (100 mL), dried over Na₂SO₄, filtered and concentrated under reduced pressure to give 6-(4-chlorophenyl)thiazolo[4,5-c]pyridin-2-amine (1.62 g) as a solid, which was used without further purification. LCMS (ESI, m/z): 262 [M+H], ¹H NMR (400 MHz, DMSO-d₆): δ 8.62 (s, 1H), 8.38 (s, 1H), 8.07 (d, J=8.6 Hz, 2H), 7.87 (s, 2H), 7.51 (d, J=8.6 Hz, 2H).

### Intermediate 67: (1r,4r)-4-(2-aminothiazolo[4,5-b]pyrazin-6-yl)cyclohexane-1-carbonitrile

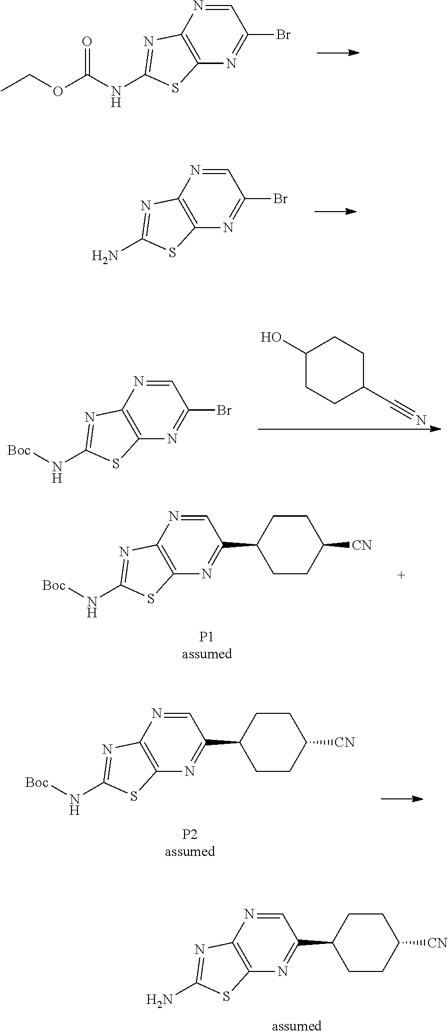

Step 1: A solution of ethyl (6-bromothiazolo[4,5-b]pyrazin-2-yl)carbamate (10 g, 33 mmol) in a mixture of MeOH (100 mL) and 2 M aqueous NaOH (100 mL) was heated at 80° C. overnight. The MeOH was removed under reduced pressure and the aqueous residue adjusted to pH 7 with 6 M aqueous HCl. The resulting precipitate was collected by filtration and dried to afford 6-bromothiazolo[4,5-b]pyrazin-2-amine (6.5 g, 85% yield) as a yellow solid. LCMS (ESI, m/z): 231 and 233 [M+H]⁺. ¹H NMR (400 MHz, DMSO-d₆) δ 8.59 (s, 2H), 8.37 (s, 1H).

Step 2: To a solution of 6-bromothiazolo[4,5-b]pyrazin-2-amine (6.5 g, 28 mmol), DMAP (0.69 g, 5.6 mmol) and TEA (8.5 g, 84 mmol) in DMF (18 mL) was added (Boc)₂O (12 g, 56 mmol) and the mixture was stirred at room temperature overnight. The mixture was poured into water (200 mL), extracted with EtOAc (80 mL×3), and the combined organic layers were washed with brine (100 mL), dried over Na₂SO₄, filtered, and concentrated under reduced pressure. The residue was purified by silica gel chromatography, eluting with DCM/MeOH (1/0 to 50/1, v/v) to afford tert-butyl (6-bromothiazolo[4,5-b]pyrazin-2-yl)carbamate (7.7 g, 83% yield) as a yellow solid. LCMS (ESI, m/z): 331 and 333 [M+H]⁺. ¹H NMR (400 MHz, DMSO-d₆) δ 12.5 (s, 1H), 8.71 (s, 1H), 1.53 (s, 9H).

Step 3: Section A: To a solution of 4-hydroxycyclohexane-1-carbonitrile (189 mg, 1.51 mmol) in 1,4-dioxane (5 mL) was added 5,7-di-tert-butyl-3-phenylbenzo[d]oxazol-3-ium tetrafluoroborate (597 mg, 1.51 mmol) and the mixture was stirred at room temperature for 10 min under a nitrogen atmosphere. Pyridine (119 mg, 1.51 mmol) was added and stirring was continued at room temperature for a further 15 min. A white solid precipitated out during this time.

Section B: To a solution of tert-butyl (6-bromothiazolo[4,5-b]pyrazin-2-yl)carbamate (200 mg, 0.604 mmol) and quinuclidine (118 mg, 1.06 mmol) in DMA (5 mL) at room temperature under a nitrogen atmosphere was added (dtpby)NiBr₂ (58.8 mg, 0.121 mmol) and [Ir(dtbbpy)(ppy)₂][PF₆](55.2 mg, 0.0604 mmol). Section A was filtered and the filtrate was injected through a syringe filter into Section B. The resulting mixture was irradiated under 450 nm blue LED modules with stirring at room temperature for 16 h under a nitrogen atmosphere.

The mixture was poured into water (50 mL), extracted with EtOAc (40 mL×3), and the combined organic layers were washed with brine (50 mL), dried over Na₂SO₄, filtered, and concentrated under reduced pressure. The residue was purified by silica gel chromatography, eluting with DCM/MeOH (1/0 to 80/1, v/v) then prep-HPLC to afford tert-butyl (6-((1s,4s)-4-cyanocyclohexyl)thiazolo[4,5-b]pyrazin-2-yl)carbamate (32 mg, 13% yield) as a white solid and tert-butyl (6-((1r,4r)-4-cyanocyclohexyl)thiazolo[4,5-b]pyrazin-2-yl)carbamate (40 mg, 17%) as a white solid.

tert-Butyl (6-((1s,4s)-4-cyanocyclohexyl)thiazolo[4,5-b]pyrazin-2-yl)carbamate (first eluting peak from p-HPLC): LCMS (ESI, m/z): 360 [M+H]⁺; ¹H NMR (400 MHz, DMSO-d₆) δ 12.2 (s, 1H), 8.46 (s, 1H), 3.27-3.21 (m, 1H), 2.97-2.87 (m, 1H), 2.06-1.96 (m, 2H), 1.95-1.88 (m, 2H), 1.87-1.69 (m, 4H), 1.52 (s, 9H).

tert-Butyl (6-((1r,4r)-4-cyanocyclohexyl)thiazolo[4,5-b]pyrazin-2-yl)carbamate (second eluting peak from p-HPLC): LCMS (ESI, m/z): 360 [M+H]⁺; ¹H NMR (400 MHz, DMSO-d₆) δ 8.36 (s, 1H), 2.92-2.73 (m, 2H), 2.19-2.08 (m, 2H), 1.95-1.86 (m, 2H), 1.73-1.59 (m, 4H), 1.50 (s, 9H).

Step 4: A mixture of tert-butyl (6-((1r,4r)-4-cyanocyclohexyl)thiazolo[4,5-b]pyrazin-2-yl)carbamate (38 mg, 0.11 mmol) and 4 M HCl in 1,4-dioxane (3 mL) was stirred at room temperature overnight. The mixture was adjusted to pH 8 with a saturated aqueous NaHCO₃ solution, extracted with DCM (30 mL×4), and the combined organic layers washed with brine (30 mL), dried over Na₂SO₄, filtered, and concentrated under reduced pressure to afford (1r,4r)-4-(2-aminothiazolo[4,5-b]pyrazin-6-yl)cyclohexane-1-carbonitrile (20 mg, 74%) as a yellow solid. LCMS (ESI, m/z): 260 [M+H]⁺.

### Intermediate 68: 6-(4-methoxytetrahydro-2H-pyran-4-yl)thiazolo[4,5-b]pyrazin-2-amine

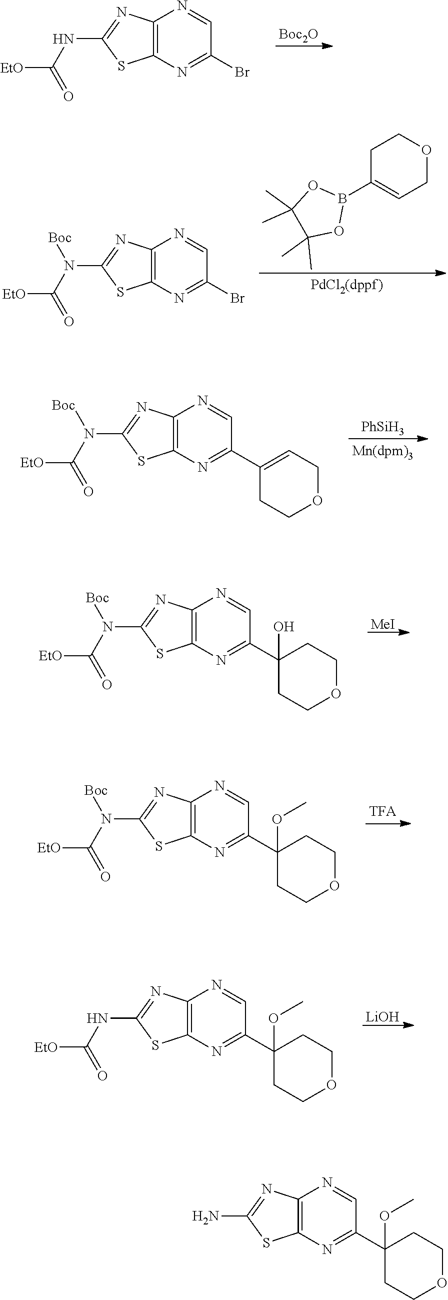

Step 1: A mixture of ethyl (6-bromothiazolo[4,5-b]pyrazin-2-yl)carbamate (2 g, 7 mmol), (Boc)₂O (4 g, 5 mL, 0.02 mol), DMAP (0.4 g, 3 mmol) and TEA (2 g, 3 mL, 0.02 mol) in DCM (40 mL) was stirred for 16 h at 25° C. The resulting mixture was concentrated under reduced pressure, and the residue was purified by silica gel column chromatography, eluting with 0-30% EtOAc in pet ether to afford tert-butyl ethyl (6-bromothiazolo[4,5-b]pyrazin-2-yl)iminodicarbonate (800 mg, 30% yield) as a white solid.

Step 2: A mixture of 4-(4,4,5,5-Tetramethyl-1,3,2-dioxaborolan-2-yl)-3,6-dihydro-2H-pyran (500 mg, 2.38 mmol), tert-butyl ethyl (6-bromothiazolo[4,5-b]pyrazin-2-yl)iminodicarbonate (800 mg, 1.98 mmol), PdCl2(dppf)-CH₂Cl₂ adduct (162 mg, 198 μmol), and K₂CO₃ (823 mg, 5.95 mmol) in 1,4-dioxane (5 mL) and H₂O (0.5 mL) was stirred at 100° C. for 12 h under a nitrogen atmosphere. The reaction mixture was cooled to room temperature and concentrated under reduced pressure. The residue was purified by silica gel column chromatography, eluting with EtOAc/pet ether (0-40%) to afford tert-butyl ethyl (6-(3,6-dihydro-2H-pyran-4-yl)thiazolo[4,5-b]pyrazin-2-yl)iminodicarbonate (680 mg, 84% yield) as a white solid.

Step 3: A mixture of tert-butyl ethyl (6-(3,6-dihydro-2H-pyran-4-yl)thiazolo[4,5-b]pyrazin-2-yl)iminodicarbonate (400 mg, 984 mol), Mn(dpm)₃ (59.5 mg, 98.4 μmol), and phenylsilane (320 mg, 366 μL, 2.95 mmol) in isopropanol (9 mL) and DCM (1 mL) was stirred for 18 h at 25° C. The mixture was concentrated under reduced pressure, and the residue was purified by silica gel column chromatography, eluting with MeOH and DCM (0-15%) to afford tert-butyl ethyl (6-(4-hydroxytetrahydro-2H-pyran-4-yl)thiazolo[4,5-b]pyrazin-2-yl)iminodicarbonate (150 mg, 36% yield) as a yellow solid.

Step 4: To a solution of tert-butyl ethyl (6-(4-hydroxytetrahydro-2H-pyran-4-yl)thiazolo[4,5-b]pyrazin-2-yl)iminodicarbonate (260 mg, 0.613 mmol) in THF (8 mL) was added sodium hydride (61.3 mg, 60% Wt, 1.53 mmol) at 0° C. The mixture was stirred for 30 min at 25° C. Mel (261 mg, 115 μL, 1.84 mmol) in THF (2 mL) was added at 0° C. and the mixture was allowed to warm to 25° C. and stirred for 1.5 h. The reaction mixture was quenched with water and extracted with DCM (3×25 mL). The combined organic layers were dried over anhydrous Na₂SO₄, filtered, and concentrated under reduced pressure. The residue was purified by silica gel column chromatography, eluting with EtOAc and pet ether (0-50%) to afford tert-butyl ethyl (6-(4-methoxytetrahydro-2H-pyran-4-yl)thiazolo[4,5-b]pyrazin-2-yl)iminodicarbonate (160 mg, 60% yield) as a white solid. LCMS (ESI, m/z): 339 [M+H-Boc]⁺.

Step 5: A solution of tert-butyl ethyl (6-(4-methoxytetrahydro-2H-pyran-4-yl)thiazolo[4,5-b]pyrazin-2-yl)iminodicarbonate (140 mg, 319 μmol) and TFA (1 g, 1 mL, 0.01 mol) in DCM (2 mL) was stirred for 12 h at 25° C. The resulting mixture was concentrated under reduced pressure to afford ethyl (6-(4-methoxytetrahydro-2H-pyran-4-yl)thiazolo[4,5-b]pyrazin-2-yl)carbamate (180 mg, crude) as a yellow oil. LCMS (ESI, m/z): 339 [M+H]⁺.

Step 6: A mixture of ethyl (6-(4-methoxytetrahydro-2H-pyran-4-yl)thiazolo[4,5-b]pyrazin-2-yl)carbamate (110 mg, 325 mol) and LiOH (77.9 mg, 3.25 mmol) in MeOH (1.5 mL) and H₂O (0.5 mL) was stirred for 5 h at 80° C. The resulting mixture was cooled to room temperature and concentrated under reduced pressure. The residue was purified by reverse phase chromatography (0-100% MeOH in water) to afford 6-(4-methoxytetrahydro-2H-pyran-4-yl)thiazolo[4,5-b]pyrazin-2-amine (60 mg, 69% yield) as a white solid. LCMS (ESI, m/z): 267 [M+H]⁺.

### Intermediate 17: 4-Bromo-6-methyl-N-(6-(pyridin-4-yl)thiazolo[4,5-b]pyridin-2-yl)nicotinamide

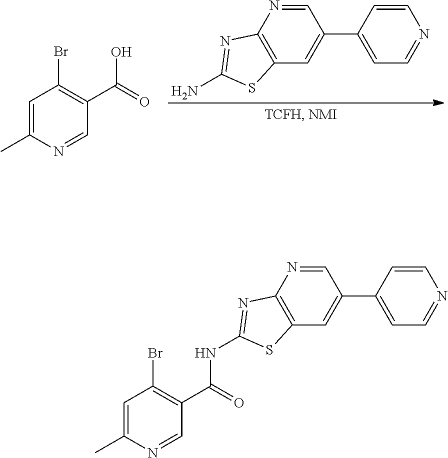 [SMILES: Cc1cc(Br)c(C(=O)O)cn1 ; Nc1nc2ncc(-c3ccncc3)cc2s1 ; Cc1cc(Br)c(C(=O)Nc2nc3ncc(-c4ccncc4)cc3s2)cn1]

To a solution of 4-bromo-6-methylnicotinic acid (400 mg, 1.82 mmol) in NMP (10 mL) was added TCFH (780 mg, 2.80 mmol), NMI (600 mg, 7.40 mmol) and 6-(pyridin-4-yl)thiazolo[4,5-b]pyridin-2-amine (211 mg, 0.91 mmol) and the mixture was stirred at room temperature overnight. The mixture was diluted with water (200 mL), extracted with EtOAc (100 mL×4) and the combined organic layers washed with brine (50 mL×2), dried over Na₂SO₄, filtered and concentrated under reduced pressure. The residue was purified by silica gel chromatography (DCM/MeOH=50/1-20/1, v/v) to afford 4-bromo-6-methyl-N-(6-(pyridin-4-yl)thiazolo[4,5-b]pyridin-2-yl)nicotinamide (100 mg, 25%) as a white solid. LCMS (ESI, m/z): 426, 428 [M+H]⁺, ¹H NMR (400 MHz, DMSO-d₆) δ 13.5 (s, 1H), 9.05 (d, J=2.2 Hz, 1H), 8.99 (d, J=2.4 Hz, 1H), 8.74 (s, 1H), 8.71-8.69 (m, 2H), 7.87-7.85 (m, 2H), 7.80 (s, 1H), 2.55 (s, 3H).

TABLE D
The following intermediates were prepared using a similar procedure to that described
for Intermediate 17

| Intermediate | | | |
|---|---|---|---|
| Number | Structure | Compound Name | Characterization Data |
| 18 | 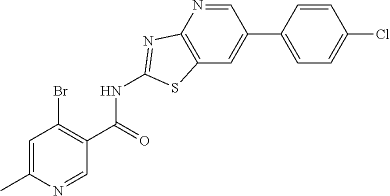 [SMILES: Cc1cc(Br)c(C(=O)Nc2nc3ncc(-c4ccc(Cl)cc4)cc3s2)cn1] | 4-Bromo-N-(6-(4- chlorophenyl)thiazolo [4,5-b]pyridin-2- yl)-6- methylnicotinamide | LCMS (m/z): 458, 460 [M + H]⁺. ¹H NMR (400 MHz, DMSO-d₆) δ 8.91 (d, J = 2.4 Hz, 1H), 8.84 (d, J = 2.4 Hz, 1H), 8.73 (s, 1H), 7.84 (d, J = 8.6 Hz, 2H), 7.79 (s, 1H), 7.59 (d, J = 8.6 Hz, 2H), 2.55 (s, 3H) |
| 19 | 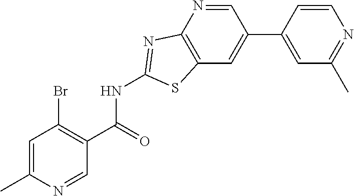 | 4-Bromo-6-methyl- N-(6-(2- methylpyridin-4- yl)thiazolo[4,5-b]pyridin 2-yl)nicotinamide | LCMS (m/z): 441, 443 [M + H]⁺. |
| 20 | 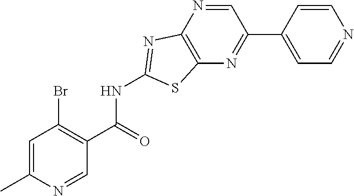 [SMILES: Cc1cc(Br)c(C(=O)Nc2nc3ncc(-c4ccncc4)nc3s2)cn1] | 4-bromo-6-methyl- N-(6-(pyridin-4- yl)thiazolo[4,5- b]pyrazin-2- yl)nicotinamide | LCMS (m/z): 428, 430 [M + H]⁺. ¹H NMR (400 MHz, DMSO-d₆) δ 9.37 (s, 1H), 8.80-8.72 (m, 3H), 8.21- 8.14 (m, 2H), 7.79 (s, 1H), 2.55 (s, 3H) |

### Intermediate 21: 4-Chloro-6-methyl-N-(6-(pyridin-4-yl)thiazolo[4,5-b]pyridin-2-yl)nicotinamide

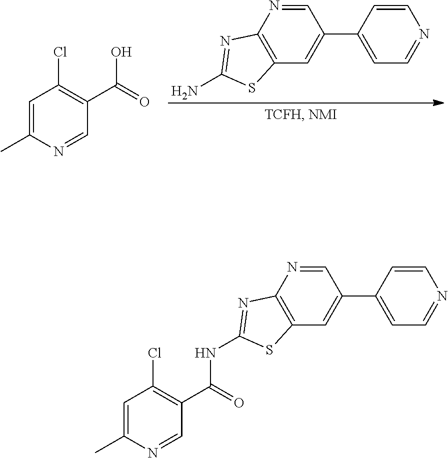 [SMILES: Cc1cc(Cl)c(C(=O)O)cn1 ; Nc1nc2ncc(-c3ccncc3)cc2s1 ; Cc1cc(Cl)c(C(=O)Nc2nc3ncc(-c4ccncc4)cc3s2)cn1]

A mixture of 4-chloro-6-methylnicotinic acid (30 mg, 0.17 mmol), 6-(pyridin-4-yl)thiazolo[4,5-b]pyridin-2-amine (48 mg, 0.21 mmol), TCFH (74 mg, 0.26 mmol) and NMI (57 mg, 0.7 mmol) in DMF (0.5 mL) and ACN (0.5 mmol) was stirred at room temperature overnight. The resulting precipitate was collected by filtration, washed with Pet. ether/EtOAc (50/1, 10 mL), ACN (10 mL) and dried under reduced pressure to give 4-chloro-6-methyl-N-(6-(pyridin-4-yl)thiazolo[4,5-b]pyridin-2-yl)nicotinamide (15 mg, 22%) as a brown solid. LCMS (ESI, m/z): 382 [M+H]⁺, ¹H NMR (400 MHz, DMSO-d₆) δ 13.5 (s, 1H), 9.02 (dd, J=22.8, 2.4 Hz, 2H), 8.80 (s, 1H), 8.72-8.66 (m, 2H), 7.89-7.80 (m, 2H), 7.65 (s, 1H), 2.57 (s, 3H).

### Intermediate 22: 4-chloro-6-methyl-N-(6-(pyridin-4-yl)thiazolo[4,5-b]pyrazin-2-yl)nicotinamide

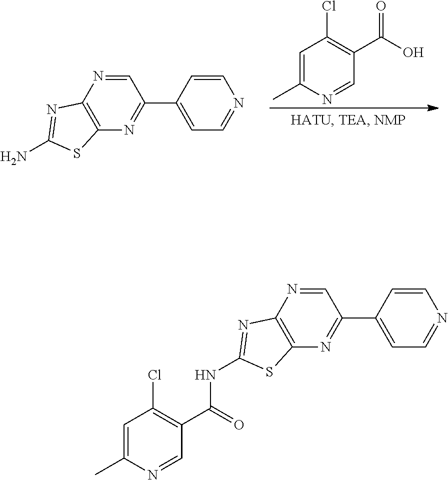

A solution of 6-(pyridin-4-yl)-[1,3]thiazolo[4,5-b]pyrazin-2-amine (1.0 g, 4.3 mmol) and 4-chloro-6-methylpyridine-3-carboxylic acid (374 mg, 2.2 mmol) was treated with TEA (1.32 g, 13.1 mmol) followed by the addition of HATU (2.0 g, 5.2 mmol) in portions at 0° C. under a nitrogen atmosphere. The mixture was stirred at 0° C. for 30 minutes under a nitrogen atmosphere. The resulting mixture was purified directly by prep-HPLC (10-50% ACN in water with 0.1% FA modifier) to afford 4-chloro-6-methyl-N-[6-(pyridin-4-yl)-[1,3]thiazolo[4,5-b]pyrazin-2-yl]pyridine-3-carboxamide (320.1 mg, 36.4% yield) as a light yellow solid. LCMS (ESI): [M+H]⁺=383. ¹H NMR (300 MHz, DMSO-d₆) δ 13.73 (s, 1H), 9.40 (s, 1H), 8.82 (s, 1H), 8.76-8.75 (m, 2H), 8.18-8.13 (m, 2H), 7.65 (s, 1H), 2.57 (s, 3H).

TABLE E
The following intermediates were prepared using a similar procedure to that described
for Intermediate 22

| Intermediate | | | LCMS: |
|---|---|---|---|
| Number | Structure | Compound Name | [M + H]⁺ |
| 23 | 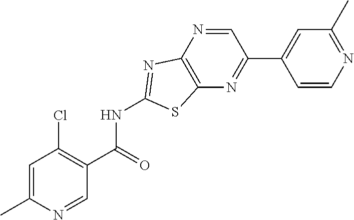 | 4-chloro-6-methyl-N-(6-(2- methylpyridin-4-yl)thiazolo[4,5- b]pyrazin-2-yl)nicotinamide | 397 |

### Intermediate 24: N-(6-Bromothiazolo[4,5-b]pyrazin-2-yl)-4-(2-methoxyphenyl)-6-methylnicotinamide

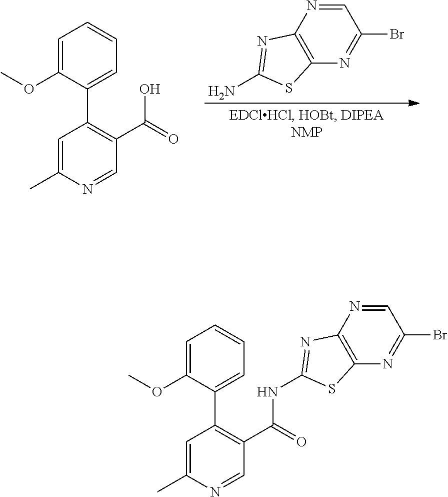 [SMILES: COc1ccccc1-c1cc(C)ncc1C(=O)O ; Nc1nc2ncc(Br)nc2s1 ; COc1ccccc1-c1cc(C)ncc1C(=O)Nc1nc2ncc(Br)nc2s1]

To a solution of 4-(2-methoxyphenyl)-6-methylnicotinic acid (2 g, 8.2 mmol) in NMP (20 mL) was added HOBt (1.67 g, 12.3 mmol), EDCI·HCl (2.36 g, 12.3 mmol) and DIPEA (3.19 g, 24.6 mmol) and the mixture was stirred at room temperature for 1 h. 6-Bromothiazolo[4,5-b]pyrazin-2-amine (1.33 g, 5.76 mmol) was added and the mixture was heated at 50° C. overnight. The mixture was diluted with water (40 mL), extracted with EtOAc (60 mL×3) and the combined organic layers washed with brine, dried over Na₂SO₄, filtered and concentrated under reduced pressure. The residue was purified by silica gel chromatography (DCM/MeOH=90/1 to 50/1, v/v) to afford N-(6-bromothiazolo[4,5-b]pyrazin-2-yl)-4-(2-methoxyphenyl)-6-methylnicotinamide (1.37 g, 33%) as a yellow solid. LCMS (ESI, m/z): 456, 458 [M+H]⁺, ¹H NMR (400 MHz, DMSO-d₆): δ 13.4 (s, 1H), 8.76 (d, J=14.0 Hz, 2H), 7.41 (ddd, J=7.4, 4.4, 2.6 Hz, 2H), 7.35 (s, 1H), 7.11 (td, J=7.6, 1.0 Hz, 1H), 7.00-6.96 (m, 1H), 3.50 (s, 3H), 2.59 (s, 3H).

### Intermediate 25: N-(6-Bromothiazolo[4,5-b]pyrazin-2-yl)-4-(5-cyano-2-methoxyphenyl)-6-methylnicotinamide

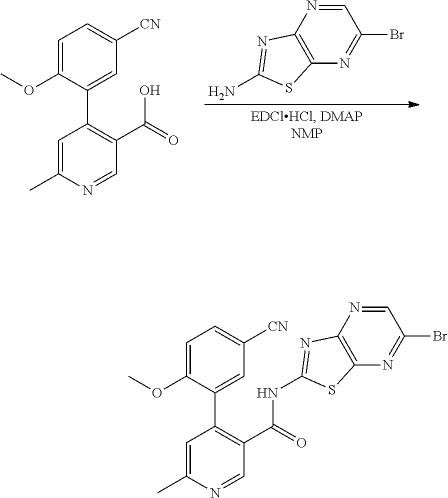 [SMILES: COc1ccc(C#N)cc1-c1cc(C)ncc1C(=O)O ; Nc1nc2ncc(Br)nc2s1 ; COc1ccc(C#N)cc1-c1cc(C)ncc1C(=O)Nc1nc2ncc(Br)nc2s1]

To a solution of 4-(5-cyano-2-methoxyphenyl)-6-methylnicotinic acid (174 mg, 0.649 mmol) in DMF (7.5 mL) was added 6-bromothiazolo[4,5-b]pyrazin-2-amine (200 mg, 0.865 mmol), EDCI·HCl (214 mg, 1.12 mmol) and DMAP (136 mg, 1.12 mmol) and the mixture was stirred at room temperature overnight. The mixture was diluted with water (75 mL), extracted with EtOAc (50 mL×6) and the combined organic layers washed with brine (120 mL), dried over Na₂SO₄, filtered and concentrated under reduced pressure. The residue was purified by prep-TLC (DCM/MeOH=30/1, v/v) to give N-(6-bromothiazolo[4,5-b]pyrazin-2-yl)-4-(5-cyano-2-methoxyphenyl)-6-methylnicotinamide (203 mg, 65%) as a pale yellow solid. LCMS (ESI, m/z): 481, 483 [M+H]⁺, ¹H NMR (400 MHz, DMSO-d₆): δ 13.5 (s, 1H), 8.80 (d, J=12.4 Hz, 2H), 7.91 (dt, J=4.6, 2.4 Hz, 2H), 7.45 (s, 1H), 7.17 (d, J=9.2 Hz, 1H), 3.59 (s, 3H), 2.60 (s, 3H).

TABLE F
The following intermediates were prepared using a similar procedure to that described
for Intermediate 25

| Intermediate | | | |
|---|---|---|---|
| Number | Structure | Compound Name | Characterization Data |
| 26 | 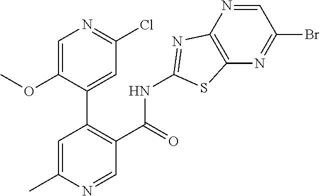 [SMILES: COc1cnc(Cl)cc1-c1cc(C)ncc1C(=O)Nc1nc2ncc(Br)nc2s1] | N-(6- Bromothiazolo[4,5- b]pyrazin-2-yl)-2′- chloro-5′-methoxy-6- methyl-[4,4′- bipyridine]-3- carboxamide | LCMS (ESI, m/z): 493, 495 [M + H]⁺. ¹H NMR (400 MHz, DMSO-d₆) δ 13.59 (s, 1H), 8.88 (s, 1H), 8.79 (s, 1H), 8.18 (s, 1H), 7.61 (s, 1H), 7.49 (s, 1H), 3.62 (s, 3H), 2.61 (s, 3 H) |

### Intermediate 27: N-(6-bromothiazolo[4,5-b]pyrazin-2-yl)-4-(2-methoxyphenyl)-2-methylpyrimidine-5-carboxamide

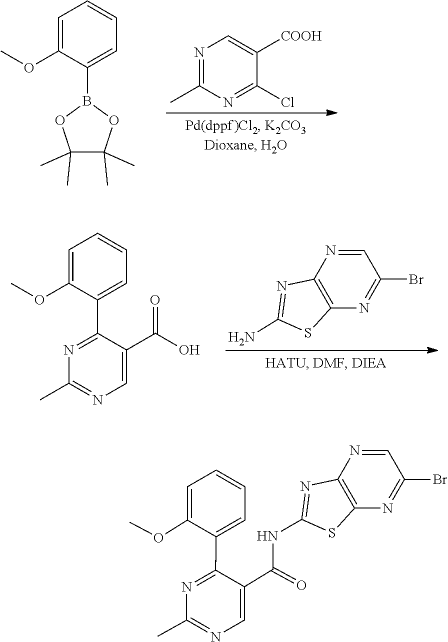

Step 1: To a stirred mixture of 2-(2-methoxyphenyl)-4,4,5,5-tetramethyl-1,3,2-dioxaborolane (300 mg, 1.2 mmol, 1.0 equiv.), 4-chloro-2-methylpyrimidine-5-carboxylic acid (219 mg, 1.2 mmol, 1.0 equiv.) and potassium carbonate (535 mg, 3.8 mmol, 3.0 equiv.) in 1,4-dioxane (1 mL) and H₂O (0.1 mL) was added Pd(dppf)Cl₂(93 mg, 0.1 mmol, 0.1 equiv.) in one portion at room temperature. The reaction mixture was charged with N2 three times at room temperature. The reaction mixture was heated to 110° C. and stirred at 110° C. for 16 h under nitrogen atmosphere. The resulting mixture was cooled to 25° C. and concentrated under reduced pressure. The residue was purified by reverse-phase flash chromatography with the following conditions: column, C18 silica gel; mobile phase, ACN in water (0.1% FA), 10% to 50% gradient in 10 min; detector, UV 254 nm to afford 4-(2-methoxyphenyl)-2-methylpyrimidine-5-carboxylic acid (160.5 mg, 51.1% yield) as a yellow solid. LCMS (ESI, m/z): [M+H]⁺=245.0.

Step 2: To a solution of methyl 4-(2-methoxyphenyl)-2-methylpyrimidine-5-carboxylic acid (160 mg, 0.6 mmol, 1.0 equiv.), 6-bromo-[1,3]thiazolo[4,5-b]pyrazin-2-amine (158 mg, 0.6 mmol, 1.0 equiv.), HATU (392 mg, 1.1 mmol, 1.5 equiv.) and DIPEA (267 mg, 2.1 mmol, 3.0 equiv.) in DMF (2 mL) in one portion at room temperature. The reaction mixture was charged with N2 three times at room temperature. The reaction mixture was stirred at 25° C. for 1 hour under nitrogen atmosphere. Then the resulting mixture was purified by reverse-phase flash chromatography directly with the following conditions: column, C18 silica gel; mobile phase, ACN in water (0.1% FA), 10% to 50% gradient in 10 min; detector, UV 254 nm. to afford N-(6-bromothiazolo[4,5-b]pyrazin-2-yl)-4-(2-methoxyphenyl)-2-methylpyrimidine-5-carboxamide (80.0 mg, 26.7% yield) as a yellow solid. LCMS (ESI, m/z): [M+H]⁺=457.0.

TABLE G
The following intermediates were prepared using a similar procedure to that described
for Intermediate 27 step 2 using Intermediate 36

| Intermediate | | | LCMS: |
|---|---|---|---|
| Number | Structure | Compound Name | [M + H]⁺ |
| 28 | 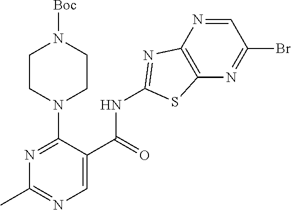 [SMILES: Cc1ncc(C(=O)Nc2nc3ncc(Br)nc3s2)c(N2CCN(C(=O)OC(C)(C)C)CC2)n1] | tert-butyl 4-(5-((6-bromothiazolo[4,5- b]pyrazin-2-yl)carbamoyl)-2- methylpyrimidin-4-yl)piperazine-1- carboxylate | 535, 537 |

### Intermediate 29: N-(6-bromothiazolo[4,5-b]pyrazin-2-yl)-2′-cyano-5′-methoxy-6-methyl-[4,4′-bipyridine]-3-carboxamide

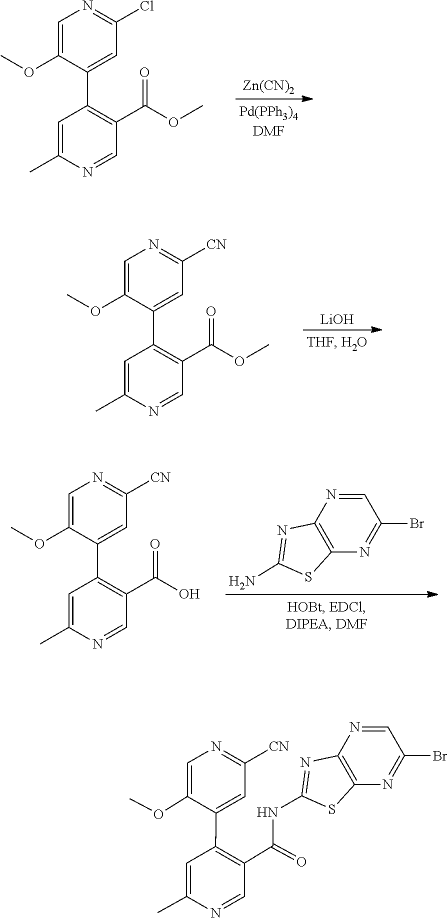

Step 1: A solution of methyl 2′-chloro-5′-methoxy-6-methyl-[4,4′-bipyridine]-3-carboxylate (400 mg, 1.4 mmol) and zinc cyanide (481 mg, 4.2 mmol) in DMF (4 mL) were added Pd(PPh₃)₄ (315 mg, 0.3 mmol) at 25° C. The reaction mixture was charged with N2 three times at 25° C., the mixture was heated to 90° C. and stirred at 90° C. for 2 h under a nitrogen atmosphere. The resulting mixture was allowed to cool to 25° C. and diluted with EtOAc (50 mL). The mixture was washed with brine and dried over anhydrous Na₂SO₄. After filtration, the filtrate was concentrated under reduced pressure. The residue was purified by silica gel column chromatography, eluting with Pet. ether/EtOAc (1/1) to afford methyl 2′-cyano-5′-methoxy-6-methyl-[4,4′-bipyridine]-3-carboxylate (350 mg, 90% yield) as a light yellow solid. LCMS (ESI): [M+H]⁺=284.

Step 2: A solution of methyl 2′-cyano-5′-methoxy-6-methyl-[4,4′-bipyridine]-3-carboxylate (350 mg, 1.2 mmol) in THF (6 mL) and water (2 mL) was added LiOH·H₂O (52 mg, 1.2 mmol) at 25° C. The reaction mixture was stirred at 25° C. for 2 h. The resulting mixture was acidified to pH 7 with 1M aq HCl. The resulting mixture was concentrated under reduced pressure. The residue was purified directly by prep-HPLC (3-14% ACN/water with 0.1% formic acid modifier) to afford 2′-cyano-5′-methoxy-6-methyl-[4,4′-bipyridine]-3-carboxylic acid (180 mg, 54% yield) as an off-white solid. LCMS (ESI): [M+H]⁺=270.

Step 3: A solution of 2′-cyano-5′-methoxy-6-methyl-[4,4′-bipyridine]-3-carboxylic acid (150 mg, 0.6 mmol) and 6-bromo-[1,3]thiazolo[4,5-b]pyrazin-2-amine (129 mg, 0.6 mmol) in DMF (3 mL) was treated with HOBt (113 mg, 0.9 mmol) and DIPEA (216 mg, 1.8 mmol) at 25° C. followed by the addition of EDCI (160 mg, 0.9 mmol) in one portion at 25° C. The reaction mixture was charged with N2 three times at 25° C. The reaction mixture was stirred at 25° C. for 12 h under a nitrogen atmosphere. The resulting mixture was purified directly by reverse-phase flash chromatography (0-100% ACN/water with 0.1% formic acid modifier) to give N-{6-bromo-[1,3]thiazolo[4,5-b]pyrazin-2-yl}-2′-cyano-5′-methoxy-6-methyl-[4,4′-bipyridine]-3-carboxamide (200 mg, 74% yield) as a light yellow solid. LCMS (ESI): [M+H]+=482, 484.

### Intermediate 69: N-(6-bromothiazolo[4,5-b]pyrazin-2-yl)-6-methyl-4-(2-methyl-2H-pyrazolo[4,3-c]pyridin-3-yl)nicotinamide

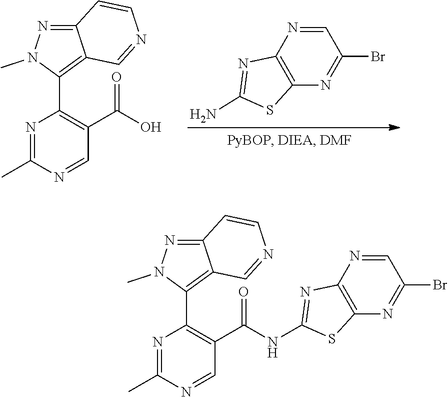 [SMILES: Cc1ncc(C(=O)O)c(-c2c3cnccc3nn2C)n1 ; Nc1nc2ncc(Br)nc2s1 ; Cc1ncc(C(=O)Nc2nc3ncc(Br)nc3s2)c(-c2c3cnccc3nn2C)n1]

A solution of 6-methyl-4-(2-methyl-2H-pyrazolo[4,3-c]pyridin-3-yl)nicotinic acid (300 mg, 1.1 mmol) in DMF (3 mL) was treated with PyBOP (873 mg, 1.6 mmol) and DIEA (867 mg, 6.7 mmol) at 25° C. for 5 minutes followed by the addition of 6-bromothiazolo[4,5-b]pyrazin-2-amine (258 mg, 1.1 mmol) in one portion at 25° C. The reaction mixture was stirred at 25° C. for 2 h. The resulting mixture was purified by reverse-phase flash chromatography (0-100% MeCN in water with 10 mmol/L NH₄HCO₃ modifier) to afford N-(6-bromothiazolo[4,5-b]pyrazin-2-yl)-6-methyl-4-(2-methyl-2H-pyrazolo[4,3-c]pyridin-3-yl)nicotinamide (300 mg, 56% yield) as a light yellow solid. LCMS (ESI, m/z): 481 [M+H]⁺.

TABLE AM
The following intermediates were prepared using a similar procedure to that
described for Intermediate 69

| Intermediate | | | LCMS |
|---|---|---|---|
| Number | Structure | Compound Name | [M + H]⁺ |
| 70 | 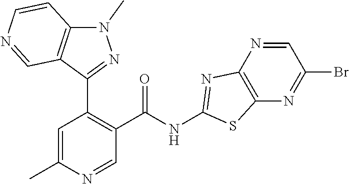 [SMILES: Cc1cc(-c2nn(C)c3ccncc23)c(C(=O)Nc2nc3ncc(Br)nc3s2)cn1] | N-(6-bromothiazolo[4,5- b]pyrazin-2-yl)-6-methyl-4-(1- methyl-1H-pyrazolo[4,3- c]pyridin-3-y1)nicotinamide | 481 |
| 71 | 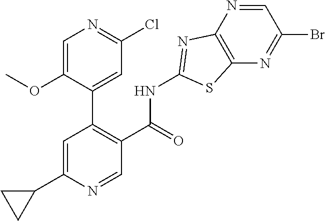 [SMILES: COc1cnc(Cl)cc1-c1cc(C2CC2)ncc1C(=O)Nc1nc2ncc(Br)nc2s1] | N-(6-bromothiazolo[4,5- b]pyrazin-2-y1)-2′-chloro-6- cyclopropyl-5′-methoxy-[4,4′- bipyridine]-3-carboxamide | 517 |

### Intermediate 30: N-(5-amino-1,3,4-thiadiazol-2-yl)-2′-chloro-5′-methoxy-6-methyl-[4,4′-bipyridine]-3-carboxamide

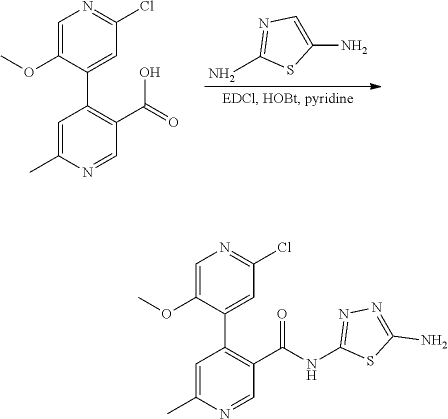 [SMILES: COc1cnc(Cl)cc1-c1cc(C)ncc1C(=O)O ; Nc1cnc(N)s1 ; COc1cnc(Cl)cc1-c1cc(C)ncc1C(=O)Nc1nnc(N)s1]

A solution of 2′-chloro-5′-methoxy-6-methyl-[4,4′-bipyridine]-3-carboxylic acid (200 mg, 0.7 mmol) and 1,3,4-thiadiazole-2,5-diamine (167 mg, 1.4 mmol) in pyridine (2.5 mL) was treated with HOBt (102 mg, 0.7 mmol) at 25° C. for 2 minutes under a nitrogen atmosphere followed by the addition of EDCI (206 mg, 1.0 mmol) in one portion at 25° C. The reaction mixture was stirred at 25° C. for 48 h under a nitrogen atmosphere. The resulting mixture was concentrated under reduced pressure. The residue was purified by reverse-phase flash chromatography (0-100% ACN/water with 0.1% NH₃—H₂O) to afford N-(5-amino-1,3,4-thiadiazol-2-yl)-2′-chloro-5′-methoxy-6-methyl-[4,4′-bipyridine]-3-carboxamide (90.0 mg, 33% yield) as a light yellow solid. LCMS (ESI): [M+H]⁺=377.

TABLE H
The following intermediates were prepared using a similar procedure to that described for Intermediate 30

| Intermediate | | | LCMS: |
|---|---|---|---|
| Number | Structure | Compound Name | [M + H]⁺ |
| 31 | 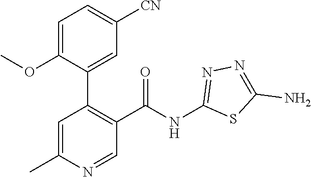 [SMILES: COc1ccc(C#N)cc1-c1cc(C)ncc1C(=O)Nc1nnc(N)s1] | N-(5-amino-1,3,4-thiadiazol- 2-yl)-4-(5-cyano-2-methoxy- phenyl)-6-methylnicotinamide | 367 |

### Intermediate 32: 2-{2′-chloro-5′-methoxy-6-methyl-[4,4′-bipyridine]-3-amido}imidazo[2,1-b][1,3,4]thiadiazole-6-carboxylic acid

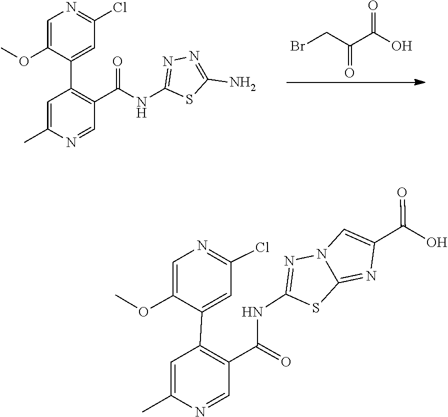 [SMILES: COc1cnc(Cl)cc1-c1cc(C)ncc1C(=O)Nc1nnc(N)s1 ; O=C(O)C(=O)CBr ; COc1cnc(Cl)cc1-c1cc(C)ncc1C(=O)Nc1nn2cc(C(=O)O)nc2s1]

Into a 250 mL 3-necked round-bottom flask were added N-(5-amino-1,3,4-thiadiazol-2-yl)-2′-chloro-5′-methoxy-6-methyl-[4,4′-bipyridine]-3-carboxamide (2.5 g, 6.6 mmol), DBU (3.0 g, 20 mmol) and 3-bromo-2-oxopropanoic acid (3.3 g, 20 mmol) at 25° C. The reaction mixture was heated at 110° C. for 1 h. The resulting mixture was allowed to cool to 25° C. and concentrated under reduced pressure. The residue was purified by trituration with water (300 mL). After filtration, the filter cake was washed with water (2×100 mL). The solid was collected and concentrated under reduced pressure to afford 2-{2′-chloro-5′-methoxy-6-methyl-[4,4′-bipyridine]-3-amido}imidazo[2,1-b][1,3,4]thiadiazole-6-carboxylic acid (2.3 g, crude) as a light brown solid. The crude product was used in the next step directly without further purification. LCMS (ESI, m/z): [M+H]=445.

### Intermediate 33: 2-(2-aminoimidazo[2,1-b][1,3,4]thiadiazol-6-yl)propan-1-ol

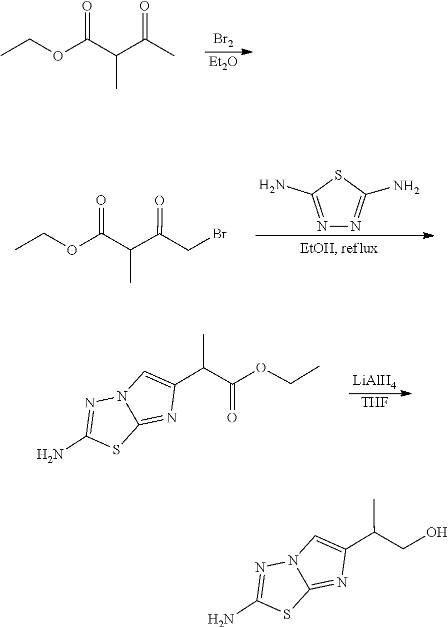

Step 1: To a stirred solution of ethyl 2-methyl-3-oxobutanoate (5.0 g, 35 mmol) in Et₂O (50 mL) was added bromine (6.1 g, 38 mmol) dropwise at −78° C. under a nitrogen atmosphere. The resulting mixture was slowly warmed to 25° C. and stirred at 25° C. for 12 h. The mixture was basified to pH 8 with saturated aq NaHCO₃. The resulting mixture was extracted with DCM (3×100 mL). The combined organic layers were washed with brine (100 mL) and dried over anhydrous Na₂SO₄. After filtration, the filtrate was concentrated under reduced pressure to afford ethyl 4-bromo-2-methyl-3-oxobutanoate (6.9 g, crude) as a brown oil. The crude product was used in the next step directly without further purification. GCMS: 222.

Step 2: A mixture of ethyl 4-bromo-2-methyl-3-oxobutanoate (3.0 g, 13 mmol) and 1,3,4-thiadiazole-2,5-diamine (780 mg, 6.7 mmol) in EtOH (50 mL) was stirred at 90° C. for 12 h under a nitrogen atmosphere. The resulting mixture was slowly cooled to 25° C. and purified by reverse-phase flash chromatography (5-30% ACN/water with 0.1% formic acid modifier) to afford ethyl 2-(2-aminoimidazo[2,1-b][1,3,4]thiadiazol-6-yl)propanoate (208 mg, 6.1% yield) as a brown oil. LCMS (ESI): [M+H]=241.1

Step 3: To a stirred solution of ethyl 2-(2-aminoimidazo[2,1-b][1,3,4]thiadiazol-6-yl)propanoate (198 mg, 0.8 mmol) in THF (5 mL) was added LiAlH₄ (31 mg, 0.8 mmol) in portions at −78° C. under a nitrogen atmosphere. The reaction mixture was slowly warmed to 0° C. and quenched by the addition of Na₂SO₄·10H₂O (2 mL) at 0° C. After filtration, the filter cake was washed with DCM (3×10 mL). The combined organic layers were washed with water (3×10 mL) and dried over anhydrous Na₂SO₄. After filtration, the filtrate was concentrated under reduced pressure. The crude product (147 mg) was purified by prep-HPLC (2-14% ACN/water with 10 mmol/L NH₄HCO₃ modifier) to afford 2-{2-aminoimidazo[2,1-b][1,3,4]thiadiazol-6-yl}propan-1-ol (23.2 mg, 14.1% yield) as a colorless solid. LCMS (ESI): [M+H]⁺=199.

### Intermediate 34: 4-(1-(tert-butoxycarbonyl)piperidin-4-yl)-6-methylnicotinic acid

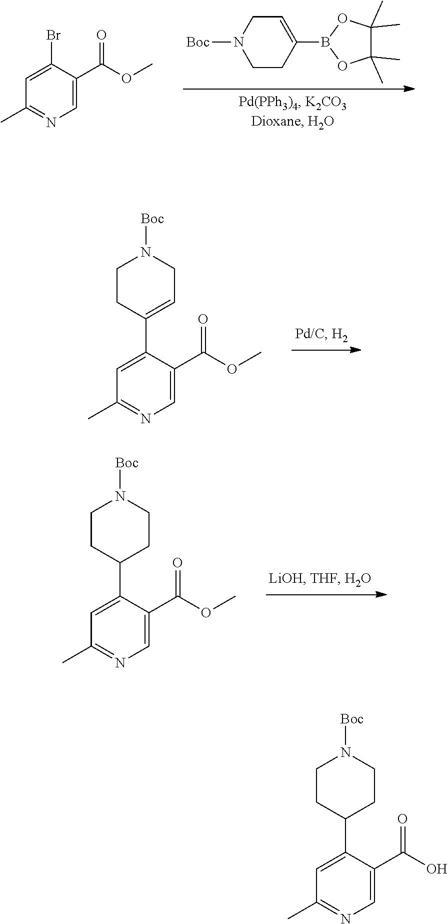

Step 1: To a stirred solution of methyl 4-bromo-6-methylpyridine-3-carboxylate (500 mg, 2.2 mmol) and tert-butyl 4-(4,4,5,5-tetramethyl-1,3,2-dioxaborolan-2-yl)-3,6-dihydro-2H-pyridine-1-carboxylate (806 mg, 2.6 mmol) in 1,4-dioxane (5 mL) and water (0.5 mL) were added Pd(PPh₃)₄ (251 mg, 0.2 mmol) and K₂CO₃ (907 mg, 6.5 mmol) in portions at room temperature under a nitrogen atmosphere. The resulting mixture was heated to 80° C. and stirred at 80° C. for 2 h. The resulting mixture was cooled to room temperature and diluted with water (50 mL). The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by silica gel column chromatography, eluting with Pet. ether/EtOAc (2:1) to afford 1-tert-butyl 3′-methyl 6′-methyl-3,6-dihydro-2H-[4,4′-bipyridine]-1,3′-dicarboxylate (700.0 mg, 97% yield) as a white solid. LCMS (ESI, m/z): [M+H]=333.

Step 2: To a mixture of 1-tert-butyl 3′-methyl 6′-methyl-3,6-dihydro-2H-[4,4′-bipyridine]-1,3′-dicarboxylate (650 mg, 2.0 mmol) and Pd/C (10%, 70 mg) was added MeOH (10 mL). The mixture was stirred under a hydrogen atmosphere at 25° C. for 2 h. The resulting solution was filtered and concentrated under reduced pressure to afford methyl 4-[1-(tert-butoxycarbonyl)piperidin-4-yl]-6-methylpyridine-3-carboxylate (600.0 mg, crude) as a white solid. LCMS (ESI, m/z): [M+H]=335.

Step 3: To a stirred solution of methyl 4-[1-(tert-butoxycarbonyl)piperidin-4-yl]-6-methylpyridine-3-carboxylate (550 mg, 1.64 mmol) in THF (4.5 mL) was added a solution of LiOH·H₂O (207 mg, 4.9 mmol) in water (2 mL) dropwise at room temperature. The resulting mixture was stirred at room temperature for 2 h. The resulting mixture was concentrated under reduced pressure. The crude product was used without further purification. LCMS (ESI, m/z): [M+H]⁺=321.

### Intermediate 35: 4-(4-(tert-butoxycarbonyl)piperazin-1-yl)-6-cyanonicotinic acid

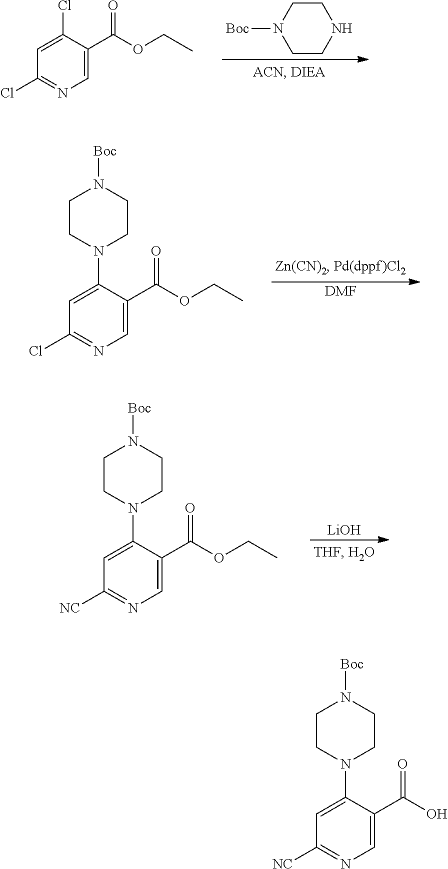

Step 1: To a stirred solution of ethyl 4,6-dichloropyridine-3-carboxylate (900 mg, 4.1 mmol) and tert-butyl piperazine-1-carboxylate (800 mg, 4.3 mmol) in ACN (9 mL) was added DIPEA (2 mL, 12.3 mmol) dropwise at 0° C. under a nitrogen atmosphere. The resulting mixture was heated and stirred at 80° C. for 12 h. The resulting mixture was cooled to room temperature and concentrated under reduced pressure. The residue was purified by silica gel column chromatography, eluting with DCM/EtOAc (4/1) to afford tert-butyl 4-[2-chloro-5-(ethoxycarbonyl)pyridin-4-yl]piperazine-1-carboxylate (1.4 g, 92.5% yield) as a colorless solid. LCMS (ESI, m/z): [M+H]⁺=370.

Step 2: To a stirred solution of tert-butyl 4-[2-chloro-5-(ethoxycarbonyl)pyridin-4-yl]piperazine-1-carboxylate (100 mg, 0.3 mmol) and Zn(CN)₂ (66 mg, 0.6 mmol) in DMF (3 mL) were added Zn (55 mg, 0.9 mmol) and Pd(dppf)Cl₂ (316 mg, 0.4 mmol) in portions at room temperature under a nitrogen atmosphere. The resulting mixture was stirred at 100° C. for 12 h. The resulting mixture was cooled to room temperature and filtered. The filtrate was concentrated under reduced pressure. The crude product mixture was used in the next step directly without further purification. LCMS (ESI, m/z): [M+H]⁺=361.

Step 3: To a stirred solution of tert-butyl 4-[2-cyano-5-(ethoxycarbonyl)pyridin-4-yl]piperazine-1-carboxylate (80 mg, 0.2 mmol) in DMF (1.5 mL) and water (0.5 mL) was added LiOH·H₂O (16 mg, 0.7 mmol) in one portion at room temperature. The resulting mixture was heated at 40° C. for 12 h. The resulting mixture was purified by reverse-phase flash chromatography (5%-50% ACN/water with 0.1% formic acid modifier) to give 4-[4-(tert-butoxycarbonyl)piperazin-1-yl]-6-cyanopyridine-3-carboxylic acid (35.0 mg, 47.4% yield) as a brown solid. LCMS (ESI, m/z): [M+H]=333.

### Intermediate 36: lithium 4-(4-(tert-butoxycarbonyl)piperazin-1-yl)-2-methylpyrimidine-5-carboxylate

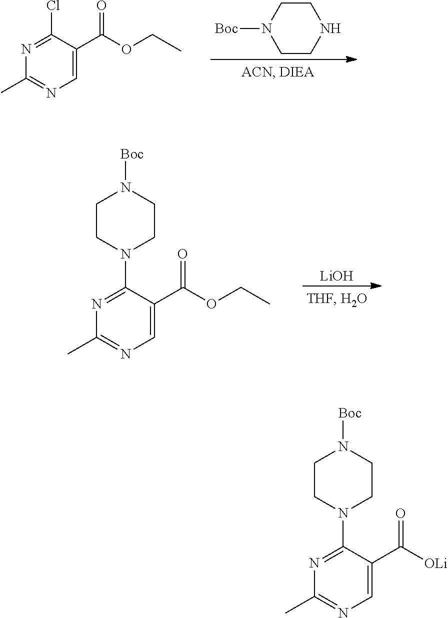

Step 1: To a stirred solution of ethyl 4-chloro-2-methylpyrimidine-5-carboxylate (100 mg, 0.5 mmol) and tert-butyl piperazine-1-carboxylate (102 mg, 0.5 mmol) in ACN (1 mL) was added DIPEA (0.3 mL, 1.5 mmol) dropwise at 0° C. under a nitrogen atmosphere. The resulting mixture was warmed to room temperature and stirred at room temperature for 12 h. The resulting mixture was concentrated under reduced pressure. The residue was purified by reverse-phase flash chromatography (10-30% ACN/water with 0.1% formic acid modifier) to afford ethyl 4-[4-(tert-butoxycarbonyl)piperazin-1-yl]-2-methylpyrimidine-5-carboxylate (160.0 mg, 92% yield) as a colorless semi-solid. LCMS (ESI, m/z): [M+H]=351.

Step 2: To a stirred solution of ethyl 4-[4-(tert-butoxycarbonyl)piperazin-1-yl]-2-methylpyrimidine-5-carboxylate (160 mg, 0.5 mmol) in THF (1.5 mL) was added LiOH·H₂O (64 mg, 1.5 mmol) in water (0.5 mL) dropwise at 0° C. The resulting mixture was stirred at 40° C. for 2 days. The resulting mixture was concentrated under reduced pressure. The crude product (120 mg) was used in the next step directly without further purification. LCMS (ESI, m/z): [M+H]⁺=323.

### Intermediate 37: 2-bromo-1-(2-methoxycyclopropyl)ethan-1-one

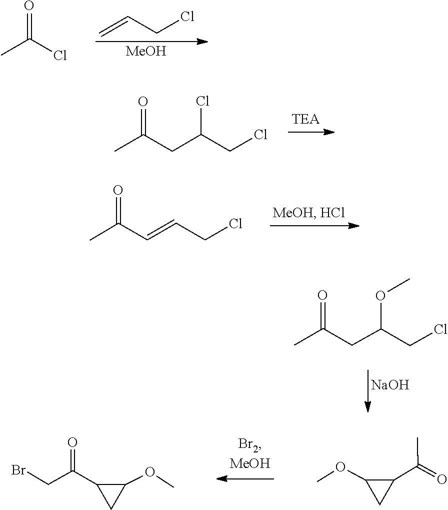

Step 1: To a stirred solution of acetyl chloride (1.0 g, 12.6 mmol) and allyl chloride (2.0 g, 25.4 mmol) in DCM (20 mL) was added AlCl3 (2.5 g, 19.0 mmol) in portions at 0° C. under a nitrogen atmosphere. The reaction mixture was stirred at 0° C. for 2 h. The mixture was warmed to 25° C. and stirred at 25° C. for 1 hour under a nitrogen atmosphere. The resulting mixture was quenched with ice water. The mixture was extracted with DCM (3×50 mL). The combined organic phase was washed with brine. The organic phase was dried over Na₂SO₄, filtered and concentrated under reduced pressure to afford 4,5-dichloropentan-2-one (1.2 g, crude) as a brown oil. The crude product was used in the next step directly without further purification. LCMS (ESI): [M+H]⁺=155.

Step 2: A solution of 4,5-dichloropentan-2-one (1.2 g, 7.7 mmol) and TEA (1.2 g, 12 mmol) in DCM (20 mL) was stirred at 45° C. for 2 h. The resulting mixture was cooled to 25° C. The resulting mixture was acidified to pH 6 with 4M aq HCl. The mixture was extracted with DCM (3×20 mL). The combined organic phase was washed with brine. The organic phase was dried over Na₂SO₄, filtered and concentrated under reduced pressure to afford (E)-5-chloropent-3-en-2-one (872 mg, crude) as a brown oil. The crude product was used in the next step directly without further purification. LCMS (ESI): [M+H]=119.

Step 3: A solution of (E)-5-chloropent-3-en-2-one (872 mg, 7.4 mmol) and HCl (0.2 mL) in MeOH (1 mL) was stirred at 25° C. for 12 h. The resulting mixture was diluted with water (10 mL) and extracted with EtOAc (3×10 mL). The combined organic phase was washed with brine. The organic phase was dried over Na₂SO₄, filtered and concentrated under reduced pressure to afford 5-chloro-4-methoxypentan-2-one (650 mg, crude) as a brown oil. LCMS (ESI): [M+H]=151.

Step 4: A solution of 5-chloro-4-methoxypentan-2-one (600 mg, 4.0 mmol) in Et₂O (5 mL) at 25° C. under a nitrogen atmosphere was treated with NaOH (1.2 mL, 10%) and H₂O (1.35 mL) at 0° C. The reaction mixture was stirred at 25° C. for 30 minutes. The resulting mixture was diluted with water (20 mL) and extracted with Et₂O (3×10 mL). The combined organic layers were washed with brine. The organic phase was dried over Na₂SO₄, filtered and concentrated under reduced pressure to afford 1-(2-methoxycyclopropyl)ethan-1-one (460 mg, crude) as a brown oil. LCMS (ESI): [M+H]⁺=115.1.

Step 5: To a stirred solution of 1-(2-methoxycyclopropyl)ethanone (460 mg, 4.0 mmol) in MeOH (5 mL) was added Br₂ (709 mg, 4.4 mmol) dropwise at 0° C. The resulting mixture was warmed to 25° C. and stirred at 25° C. for an additional 4 h. The resulting mixture was diluted with water (20 mL). The mixture was extracted with Et₂O (3×20 mL). The combined organic phase was washed with brine, dried over Na₂SO₄, filtered and concentrated under reduced pressure to afford 2-bromo-1-(2-methoxycyclopropyl)ethan-1-one (320 mg, crude) as a brown oil. LCMS (ESI): [M+H]⁺=193, 195.

### Intermediate 38: 3-(2-bromoacetyl)cyclobutan-1-one

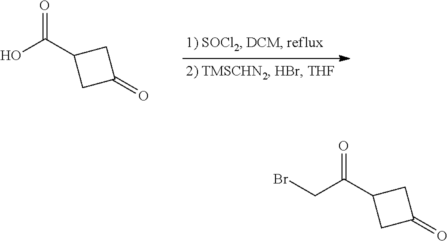 [SMILES: O=C1CC(C(=O)O)C1 ; O=C1CC(C(=O)CBr)C1]

A solution of 3-oxocyclobutane-1-carboxylic acid (3.0 g, 26 mmol) in DCM (22 mL) was treated with thionyl chloride (9.3 g, 79 mmol) for 2 minutes at 0° C. under nitrogen atmosphere. The mixture was stirred at 50° C. for 2 h under a nitrogen atmosphere. Then the reaction mixture was cooled to 25° C. and concentrated under reduced pressure and the residue was collected. The residue was dissolved in THF (15 mL). To a stirred mixture was added TMSCHN₂ (3.6 g, 31.5 mmol) dropwise at 0° C. under a nitrogen atmosphere. The mixture was stirred at 0° C. for 1 hour under a nitrogen atmosphere. To the mixture was added hydrogen bromide (4.2 g, 52.5 mmol) dropwise at 0° C. under a nitrogen atmosphere. The mixture was stirred at 0° C. for 1 hour under a nitrogen atmosphere. The mixture was concentrated under reduced pressure, diluted with water (50 mL), and extracted with EtOAc (3×50 mL). The combined organic layers were dried over anhydrous Na₂SO₄. After filtration, the filtrate was concentrated under reduced pressure. The residue was purified by silica gel column chromatography, eluting with Pet. ether/EtOAc (10/1) to afford 3-(2-bromoacetyl)cyclobutan-1-one (801 mg, 16% yield) as a yellow oil. GCMS (ESI, m/z): 190.

TABLE I
The following intermediates were prepared using a similar procedure to that described
for Intermediate 38

| Intermediate | | | LCMS: |
|---|---|---|---|
| Number | Structure | Compound Name | [M + H]⁺ |
| 39 | 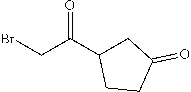 | 3-(2-bromoacetyl)cyclopentan-1- one | 205, 207 |
| 40 | 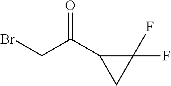 | 2-bromo-1-(2,2- difluorocyclopropyl)ethan-1-one | 199, 201 |
| 41 | 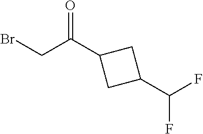 [SMILES: O=C(CBr)C1CC(C(F)F)C1] | 2-bromo-1-(3- (difluoromethyl)cyclobutyl) ethan-1-one | 227, 229 |
| 42 | 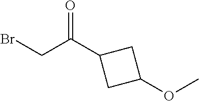 | 2-bromo-1-(3- methoxycyclobutyl)ethan-1-one | 207, 209 |
| 43 | 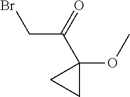 | 2-bromo-1-(1- methoxycyclopropyl)ethan-1-one | Used crude |
| 44 | 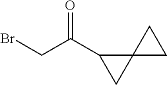 | 2-bromo-1-(spiro[2.2]pentan-1- yl)ethan-1-one | 189, 191 |
| 45 | 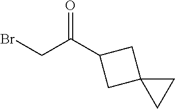 | 2-bromo-1-(spiro[2.3]hexan-5- yl)ethan-1-one | Used crude |

### Intermediate 46: 1-bromo-3-(tetrahydrofuran-3-yl)propan-2-one

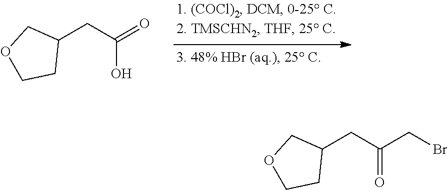 [SMILES: O=C(O)CC1CCOC1 ; O=C(CBr)CC1CCOC1]

To a stirred solution of oxolan-3-ylacetic acid (530 mg, 4.1 mmol) in DCM (10 mL) was added (COCl)₂ (1.3 g, 10 mmol) dropwise at 0° C. under a nitrogen atmosphere. The reaction mixture was stirred at 25° C. for 3 h under a nitrogen atmosphere. The resulting mixture was concentrated under reduced pressure. The residue was dissolved in THF (10 mL) under a nitrogen atmosphere, then (diazomethyl)trimethylsilane (1.4 g, 12.2 mmol) was added dropwise at 25° C. under a nitrogen atmosphere. The reaction mixture was stirred at 25° C. for 12 h. To the above mixture was added 48% HBr in water (989 mg, 12.2 mmol) dropwise at 25° C. The resulting mixture was stirred at 25° C. for an additional 2 h. The mixture was neutralized to pH 7 with saturated aq NaHCO₃ solution. The resulting mixture was extracted with EtOAc (3×20 mL). The combined organic layers were washed with brine (3×20 mL) and dried over anhydrous Na₂SO₄. After filtration, the filtrate was concentrated under reduced pressure to afford 1-bromo-3-(tetrahydrofuran-3-yl)propan-2-one (320 mg, crude) as a yellow oil.

### Intermediate 47: 1-bromo-3-(tetrahydrofuran-2-yl)propan-2-one

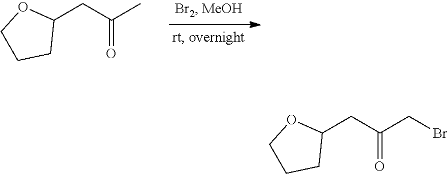

To a solution of 1-(oxolan-2-yl)propan-2-one (100 mg, 0.8 mmol) in MeOH (2 mL) was added Br₂ (125 mg, 0.8 mmol) dropwise at 0° C. The reaction mixture was slowly warmed to 25° C. and stirred for 12 h. The resulting mixture was concentrated under reduced pressure to afford 1-bromo-3-(tetrahydrofuran-2-yl)propan-2-one (135.2 mg, crude) as a yellow oil. GCMS (ESI, m/z): 206.

TABLE J
The following intermediates were prepared using a similar procedure to that described
for Intermediate 47

| Intermediate | | | LCMS: |
|---|---|---|---|
| Number | Structure | Compound Name | [M + H]⁺ |
| 48 | 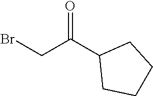 | 2-bromo-1-cyclopentylethan- 1-one | 191, 193 |
| 49 | 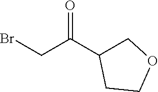 | 2-bromo-1-(tetrahydrofuran-3- yl)ethan-1-one | 193, 195 |
| 50 | 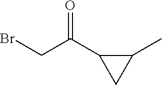 | 2-bromo-1-(2- methylcyclopropyl)ethan-1- one | 177, 179 |
| 51 | 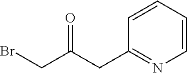 [SMILES: O=C(CBr)Cc1ccccn1] | 1-bromo-3-(pyridin-2- yl)propan-2-one | 214, 216 |
| 52 | 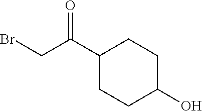 [SMILES: O=C(CBr)C1CCC(O)CC1] | 2-bromo-1-(4- hydroxycyclohexyl)ethan-1- one | 221, 223 |
| 53 | 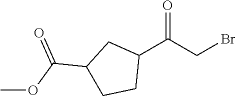 | methyl 3-(2- bromoacetyl)cyclopentane-1- carboxylate | 249, 251 |

### Intermediate 54: 1-bromo-3-(pyridin-3-yl)propan-2-one

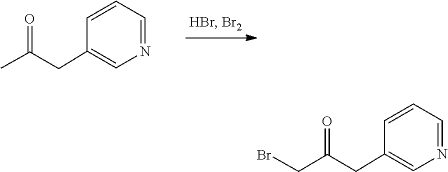

To a stirred solution of 1-(pyridin-3-yl)propan-2-one (100 mg, 0.7 mmol) in HBr (3 mL) was added Br₂ (118 mg, 0.7 mmol) dropwise at 0° C. and stirred at 0° C. for 2 h under a nitrogen atmosphere. The resulting mixture was concentrated under reduced pressure to afford 1-bromo-3-(pyridin-3-yl) propan-2-one (70 mg, crude) as a yellow oil. The crude product was used for the next step without further purification. LCMS (ESI, m/z): [M+H]⁺=213, 215.

TABLE K
The following intermediate was prepared using a similar procedure to that described
for Intermediate 54

| Intermediate | | | LCMS: |
|---|---|---|---|
| Number | Structure | Compound Name | [M + H]⁺ |
| 55 | 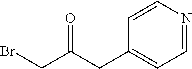 [SMILES: O=C(CBr)Cc1ccncc1] | 1-bromo-3-(pyridin-4-yl)propan-2-one | 215, 217 |

### Intermediate 72: 4,4,5,5-tetramethyl-2-(2-methyl-3,6-dihydro-2H-pyran-4-yl)-1,3,2-dioxaborolane

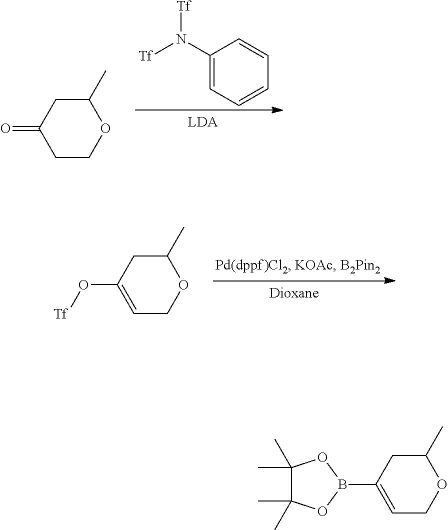

Step 1: To a stirred solution of 2-methyltetrahydro-4H-pyran-4-one (2.0 g, 0.1 mmol) in THF (20 mL) was added LDA solution (2.0 g, 0.1 mmol) dropwise at −78° C. under a nitrogen atmosphere. The reaction mixture was stirred at −78° C. for 2 h under a nitrogen atmosphere. The mixture was warmed to 0° C., and 1,1,1-trifluoro-N-phenyl-N-((trifluoromethyl)sulfonyl)-methanesulfonamide (7.0 g, 0.1 mmol) in THF (40 mL) was added dropwise at 0° C. for 15 minutes under a nitrogen atmosphere. The reaction was stirred at 0° C. for 3 h. The resulting mixture was quenched with saturated NH₄Cl solution and extracted with EtOAc (3×500 mL). The combined organic layers were washed with brine, dried over anhydrous Na₂SO₄, filtered, and concentrated under reduced pressure. The residue was purified by silica gel column chromatography, eluting with pet ether:EtOAc (20/1) to afford 2-methyl-3,6-dihydro-2H-pyran-4-yl trifluoromethanesulfonate (1.1 g, 30% yield) as a yellow solid.

Step 2: To a solution of 2-methyl-3,6-dihydro-2H-pyran-4-yl trifluoromethanesulfonate (1.2 g, 0.8 mmol) and bis(pinacolato)diborane (1.9 g, 7.3 mmol) in 1,4-dioxane (20 mL) was added Pd(dppf)Cl₂ (360 mg, 0.5 mmol) and potassium acetate (239 mg, 15.0 mmol) at 25° C. The reaction mixture was charged with N2 three times at 25° C. and stirred at 80° C. for 4 h under a nitrogen atmosphere. The resulting mixture was cooled to 25° C. and concentrated under reduced pressure. The residue was purified by silica gel column chromatography, eluting with pet ether:EtOAc (20/1) to afford 4,4,5,5-tetramethyl-2-(2-methyl-3,6-dihydro-2H-pyran-4-yl)-1,3,2-dioxaborolane (705 mg, 65% yield) as a yellow solid.

### Intermediate 73: N′-(4-(difluoromethyl)-4-methoxycyclohexylidene)-4-methylbenzenesulfonohydrazide

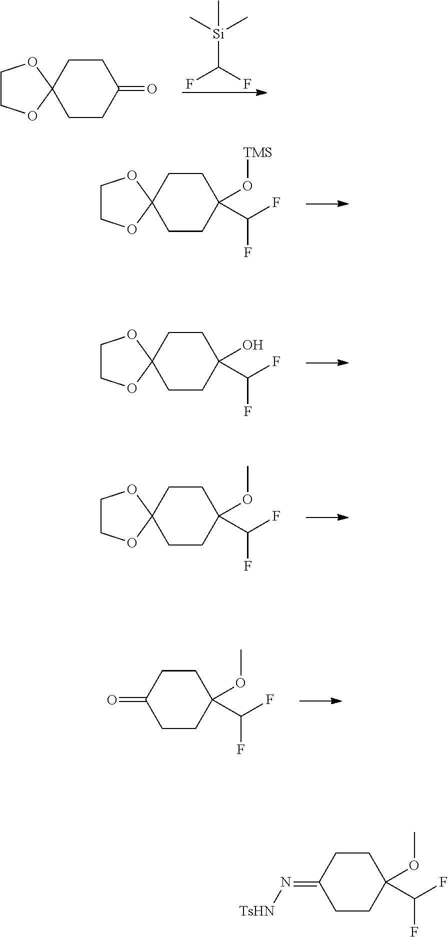

Step 1: A solution of 1,4-dioxaspiro[4.5]decan-8-one (500 mg, 3.20 mmol) and (difluoromethyl) trimethylsilane (795 mg, 6.40 mmol), CsF (48.6 mg, 0.32 mmol), and 18-crown-6 (84.6 mg, 0.32 mmol) in DME (5 mL) was stirred for 12 hours at 25° C. The resulting mixture was concentrated under reduced pressure to afford ((8-(difluoromethyl)-1,4-dioxaspiro[4.5]decan-8-yl)oxy)trimethylsilane (650 mg, 72% yield) as a yellow oil. The crude product was used in the next step directly without further purification. LCMS (ESI, m/z): 281 [M+H]⁺.

Step 2: A solution of ((8-(difluoromethyl)-1,4-dioxaspiro[4.5]decan-8-yl)oxy)trimethylsilane (1 g, 4 mmol) and TBAF (10 mL, 1 M, 6 mmol) in DME (10 mL) was stirred for 2 hours at 25° C. The resulting mixture was concentrated under reduced pressure to afford 8-(difluoromethyl)-1,4-dioxaspiro[4.5]decan-8-ol (1.00 g, 100% yield) as a yellow oil. The crude product was used in the next step directly without further purification. LCMS (ESI, m/z): 209 [M+H]⁺.

Step 3: To a stirred solution of 8-(difluoromethyl)-1,4-dioxaspiro[4.5]decan-8-ol (1.0 g, 5.0 mmol) in THF (9 mL) were added NaH (300 mg, 7.00 mmol) in portions at 0° C. The mixture was stirred for 30 min at room temperature under a nitrogen atmosphere. Iodomethane (2.00 g, 0.01 mol) was added in portions at 0° C. under a nitrogen atmosphere. The resulting mixture was stirred for 2 h at room temperature under a nitrogen atmosphere. The reaction was quenched with ice water (20 mL) at room temperature. The resulting mixture was extracted with EtOAc (3×20 mL). The combined organic layers were washed with brine (20 mL) and dried over anhydrous Na₂SO₄. After filtration, the filtrate was concentrated under reduced pressure to afford 8-(difluoromethyl)-8-methoxy-1,4-dioxaspiro[4.5]decane (960 mg, 90% yield) as a yellow solid. ¹H NMR (400 MHz, DMSO-d₆) δ 6.00 (t, J=55.1 Hz, 1H), 3.21-3.12 (m, 2H), 1.84-1.69 (m, 1H), 1.56 (d, J=9.5 Hz, 5H), 1.31 (q, J=7.3 Hz, 2H), 0.94 (t, J=7.4 Hz, 3H).

Step 4: To a stirred solution of 8-(difluoromethyl)-8-methoxy-1,4-dioxaspiro[4.5]decane (960 mg, 4.32 mmol) in THF (10 mL) were added 1M HCl (10 mL) in portions at 0° C. The resulting mixture was stirred for 16 h at room temperature under a nitrogen atmosphere. The reaction was quenched with ice water (20 mL) at room temperature. The resulting mixture was extracted with EtOAc (3×20 mL). The combined organic layers were washed with brine (20 mL) and dried over anhydrous Na₂SO₄. After filtration, the filtrate was concentrated under reduced pressure to afford 4-(difluoromethyl)-4-methoxycyclohexan-1-one (760 mg, crude) as a yellow oil. ¹H NMR (400 MHz, DMSO-d₆) δ 6.12 (t, J=54.9 Hz, 1H), 3.38 (s, 3H), 3.23-3.08 (m, 1H), 2.43 (t, J=14.0, 7.2 Hz, 1H), 2.21-2.05 (m, 2H), 1.93-1.73 (m, 2H), 1.56 (d, J=10.0 Hz, 1H), 1.39-1.14 (m, 1H).

Step 5: To a stirred solution of 4-(difluoromethyl)-4-methoxycyclohexan-1-one (760 mg, 4.27 mmol) in MeOH (15 mL) were added 4-methylbenzenesulfonic acid hydrazide (794 mg, 4.27 mmol) in portions at room temperature. The resulting mixture was stirred for 16 h at room temperature under a nitrogen atmosphere. The reaction was quenched with ice water (20 mL) at room temperature. The resulting mixture was extracted with EtOAc (3×20 mL). The combined organic layers were washed with brine (20 mL) and dried over anhydrous Na₂SO₄.

After filtration, the filtrate was concentrated under reduced pressure. The residue was purified by silica gel column chromatography, eluting with pet. ether:EtOAe (0-50%) to afford N′-(4-(difluoromethyl)-4-methoxycyclohexylidene)-4-methylbenzenesulfonohydrazide (500 mg, 34% yield) as a yellow solid. LCMS (ESI, m/z): 345 [M−H]⁻.

TABLE AN
The following intermediates were prepared using a similar procedure to that
described for Intermediate 73 starting from the commercial ketone

| Intermediate | | | LCMS |
|---|---|---|---|
| Number | Structure | Compound Name | [M + H]⁺ |
| 74 | 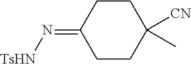 | methylcyclohexylidene)-4- methylbenzenesulfonohydrazide | 306 |
| 75 | 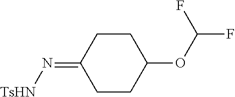 | N′-(4- (difluoromethoxy) cyclohexylidene)-4- methylbenzenesulfonohydrazide | 333 |
| 76 | 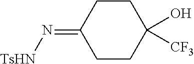 | N′-(4-hydroxy-4- (trifluoromethyl) cyclohexylidene)-4- methylbenzenesulfonohydrazide | 351 |
| 77 | 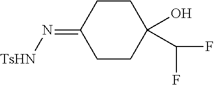 | N′-(4-(difluoromethyl)-4- hydroxycyclohexylidene)-4- methylbenzenesulfonohydrazide | 333 |
| 78 | 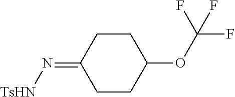 | 4-methyl-N′-(4- (trifluoromethoxy) cyclohexylidene) benzenesulfonohydrazide | 351 |

### Intermediate 79: 1,3-dioxoisoindolin-2-yl 4-cyanobicyclo[2.2.2]octane-1-carboxylate

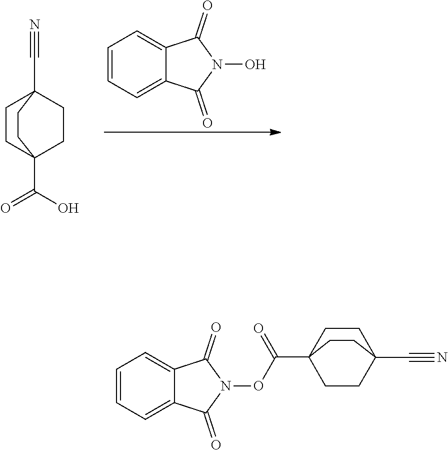 [SMILES: N#CC12CCC(C(=O)O)(CC1)CC2 ; O=C1c2ccccc2C(=O)N1O ; N#CC12CCC(C(=O)ON3C(=O)c4ccccc4C3=O)(CC1)CC2]

A solution of 4-cyanobicyclo[2.2.2]octane-1-carboxylic acid (750 mg, 4.18 mmol), DIC (528 mg, 4.18 mmol), 2-hydroxyisoindoline-1,3-dione (683 mg, 4.18 mmol) and DMAP (51 mg, 0.42 mmol) in DCM (10 mL) was stirred for 2 h at 25° C. under a nitrogen atmosphere.

The resulting mixture was concentrated under reduced pressure. The residue was purified by silica gel column chromatography, eluting with petroleum ether/EtOAc (0-30%) to afford 1,3-dioxoisoindolin-2-yl 4-cyanobicyclo[2.2.2]octane-1-carboxylate (1 g, 74% yield) as a white solid. ¹H NMR (400 MHz, Chloroform-d) δ 7.81 (dd, J=5.5, 3.1 Hz, 2H), 7.72 (dd, J=5.5, 3.1 Hz, 2H), 2.01 (tt, J=10.0, 5.2 Hz, 12H).

TABLE AO
The following intermediate was prepared using a similar procedure to that
described for Intermediate 79

| Intermediate | | | |
|---|---|---|---|
| Number | Structure | Compound Name | NMR |
| 80 | 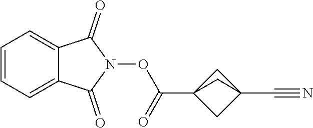 [SMILES: N#CC12CC(C(=O)ON3C(=O)c4ccccc4C3=O)(C1)C2] | 1,3-dioxoisoindolin-2-yl 3-cyanobicyclo [1.1.1]pentane- 1-carboxylate | ¹H NMR (400 MHz, Chloroform-d) δ 7.90 (dd, J = 5.5, 3.1 Hz, 2H), 7.81 (dd, J = 5.5, 3.1 Hz, 2H), 2.76 (s, 6H) |

### Intermediate 81: 4-methoxy-4-methylcyclohexan-1-ol

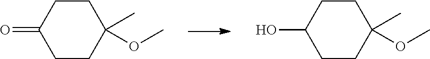

To a stirred solution of 4-methoxy-4-methylcyclohexan-1-one (300 mg, 2.11 mmol) in MeOH (6 mL) was added NaBH₄ (160 mg, 4.22 mmol) at 0° C. under a nitrogen atmosphere.

The resulting mixture was stirred for 2 h at 20° C. under a nitrogen atmosphere. The mixture was extracted with CH₂Cl₂ (4×50 mL) and the combined organic layers were washed with brine (2×8 mL) and dried over anhydrous Na₂SO₄. After filtration, the filtrate was concentrated under reduced pressure to afford crude 4-methoxy-4-methylcyclohexan-1-ol (300 mg) as yellow oil. ¹H NMR (400 MHz, DMSO-d₆): δ 4.41 (d, J=4.5 Hz, 1H), 3.34-3.29 (m, 1H), 3.05 (d, J=2.9 Hz, 3H), 1.73-1.65 (m, 2H), 1.56-1.4 (m, 2H), 1.39-1.32 (m, 2H), 1.27-1.14 m, 2H), 1.01 (s, 3H).

### Intermediate 82: 4-methoxy-4-(trifluoromethyl)cyclohexan-1-ol

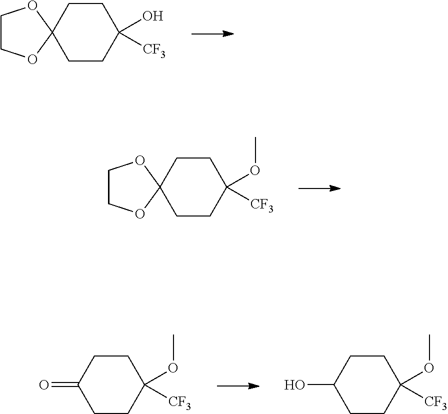 [SMILES: OC1(C(F)(F)F)CCC2(CC1)OCCO2 ; COC1(C(F)(F)F)CCC2(CC1)OCCO2 ; COC1(C(F)(F)F)CCC(=O)CC1 ; COC1(C(F)(F)F)CCC(O)CC1]

Step 1: To a stirred mixture of 8-(trifluoromethyl)-1,4-dioxaspiro[4.5]decan-8-ol (5 g, 0.02 mol) and THF (150 mL) was added NaH (2 g, 60% Wt, 0.04 mol) in portions at 0° C. under a nitrogen atmosphere. The resulting mixture was stirred for 1 h at 25° C. under a nitrogen atmosphere. To the above mixture was added Mel (1.77 g, 0.10 mol) dropwise at 0° C. The resulting mixture was stirred for an additional 12 h at 25° C. The reaction was quenched with water (100 mL) at 0° C., and the resulting mixture was extracted with EtOAc (3×200 mL). The combined organic layers were dried over anhydrous Na₂SO₄, filtered, and concentrated under reduced pressure. The residue was purified by silica gel column chromatography, eluting with pet ether:EtOAc (3/1) to afford 8-methoxy-8-(trifluoromethyl)-1,4-dioxaspiro[4.5]decane (2 g, 40% yield) as a yellow oil. ¹H NMR (300 MHz, DMSO-d₆) δ 3.88 (s, 4H), 3.35 (s, 3H), 2.03-1.89 (m, 2H), 1.76-1.56 (m, 6H).

Step 2: To a solution of 8-methoxy-8-(trifluoromethyl)-1,4-dioxaspiro[4.5]decane (2 g, 8 mmol) in THF (20 mL) was added HCl in dioxane (20 mL, 0.02 mol, 4M) at 25° C. The reaction mixture was and stirred at 25° C. for 12 h under a nitrogen atmosphere. The resulting mixture was concentrated under reduced pressure. The residue was purified by silica gel column chromatography, eluting with pet ether:EtOAc (3/1) to afford 4-methoxy-4-(trifluoromethyl)cyclohexan-1-one (1.3 g, 80% yield) as a yellow oil. ¹H NMR (400 MHz, DMSO-d₆) δ 3.45 (t, 3H), 2.49-2.40 (m, 2H), 2.21 (dtd, 4H), 2.03 (td, 2H).

Step 3: To a solution of 4-methoxy-4-(trifluoromethyl)cyclohexan-1-one (1.1 g, 5.6 mmol) in MeOH (10 mL) was added NaBH₄ (0.21 g, 5.6 mmol) at 0° C. The reaction mixture was stirred at 25° C. for 2 h under a nitrogen atmosphere. The reaction was quenched with water (100 mL) at 0° C. The resulting mixture was extracted with EtOAc (3×200 mL). The combined organic layers were dried over anhydrous Na₂SO₄, filtered, and concentrated under reduced pressure. The residue was purified by silica gel column chromatography, eluting with pet ether:EtOAc (1/1) to afford 4-methoxy-4-(trifluoromethyl)cyclohexan-1-ol (850 mg, 76% yield) as a colorless oil. ¹H NMR (300 MHz, Chloroform-d) δ 3.71-3.59 (m, 1H), 3.42 (q, J=1.3 Hz, 3H), 2.05 (ddt, J=10.5, 3.7, 2.0 Hz, 2H), 1.88 (tt, J=8.9, 7.1, 5.0 Hz, 2H), 1.66-1.47 (m, 4H).

### Intermediate 83: 5-hydroxybicyclo[2.2.1]heptane-2-carbonitrile

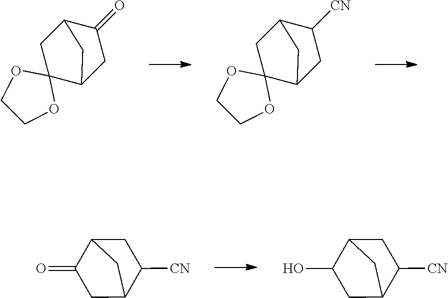 [SMILES: O=C1CC2CC1CC21OCCO1 ; N#CC1CC2CC1CC21OCCO1 ; N#CC1CC2CC1CC2=O ; N#CC1CC2CC1CC2O]

Step 1: To a stirred solution of spiro[bicyclo[2.2.1]heptane-2,2′-[1,3]dioxolan]-5-one (2.0 g, 12 mmol) and tosylmethyl isocyanide (3.00 g, 15.3 mmol) in DMF (5 mL) and EtOH (0.3 mL) was added 2-methylpropan-2-olate potassium (3.00 g, 29.7 mmol) in portions at −10° C. under a nitrogen atmosphere. The mixture was warmed to 0° C. and stirred another 1 h at 0° C. followed by 2.5 h at 22° C. The resulting mixture was extracted with diethyl ether (3×100 mL). The combined organic layers were washed with brine (2×30 mL) and dried over anhydrous Na₂SO₄. After filtration, the filtrate was concentrated under reduced pressure to afford spiro[bicyclo[2.2.1]heptane-2,2′-[1,3]dioxolane]-5-carbonitrile (2.60 g, crude) as yellow oil. The crude product mixture was used directly in the next step without further purification.

Step 2: To a stirred solution of spiro[bicyclo[2.2.1]heptane-2,2′-[1,3]dioxolane]-5-carbonitrile (2.70 g, 15 mmol) in CH₂Cl₂ (40 mL) was added TFA (20 mL) at 25° C. under a nitrogen atmosphere. The resulting mixture was stirred for additional 35 h at room temperature.

The mixture was basified to pH 8 with saturated aqueous sodium bicarbonate solution. The resulting mixture was extracted with CH₂Cl₂ (3×50 mL). The combined organic layers were washed with brine (2×20 mL) and dried over anhydrous Na₂SO₄. After filtration, the filtrate was concentrated under reduced pressure. The residue was purified by silica gel column chromatography, eluting with petroleum ether:EtOAc (10-40%) to afford 5-oxobicyclo[2.2.1]heptane-2-carbonitrile (600 mg, 35% yield over two steps) as yellow oil. ¹H NMR (400 MHz, CDCl₃) δ 3.10-2.94 (m, 2H), 2.79-2.61 (m, 1H), 2.50-2.29 (m, 2H), 2.26-2.16 (m, 1H), 2.14-2.00 (m, 1H), 1.96-1.88 (m, 1H), 1.85-1.65 (m, 1H).

Step 3: To a stirred solution of 5-oxobicyclo[2.2.1]heptane-2-carbonitrile (200 mg, 1.48 mmol) in MeOH (5 mL) was added NaBH₄ (112 mg, 2.96 mmol) in portions at 0° C. under a nitrogen atmosphere. The resulting mixture was stirred for 2 h at room temperature. The mixture was extracted with CH₂Cl₂ (3×20 mL). The combined organic layers were washed with brine (10 mL) and dried over anhydrous Na₂SO₄. After filtration, the filtrate was concentrated under reduced pressure. The residue was purified by silica gel column chromatography, eluting with petroleum ether:EtOAc (18-40%) to afford 5-hydroxybicyclo[2.2.1]heptane-2-carbonitrile (110 mg, 54% yield) as light yellow oil. ¹H NMR (400 MHz, CDCl₃) δ 4.35-4.25 (m, 1H), 2.84-2.74 (m, 1H), 2.53-2.47 (m, 1H), 2.43-2.36 (m, 1H), 2.31-2.23 (m, 1H), 2.08-1.96 (m, 1H), 1.88-1.79 (m, 1H), 1.73-1.65 (m, 1H), 1.59-1.50 (m, 1H), 1.52-1.45 (m, 1H), 1.43-1.37 (m, 1H).

### Example 1: Synthesis of N-Cyclopropyl-2-(4-(2-methoxyphenyl)-6-methylnicotinamido)imidazo[2,1-b][1,3,4]thiadiazole-6-carboxamide

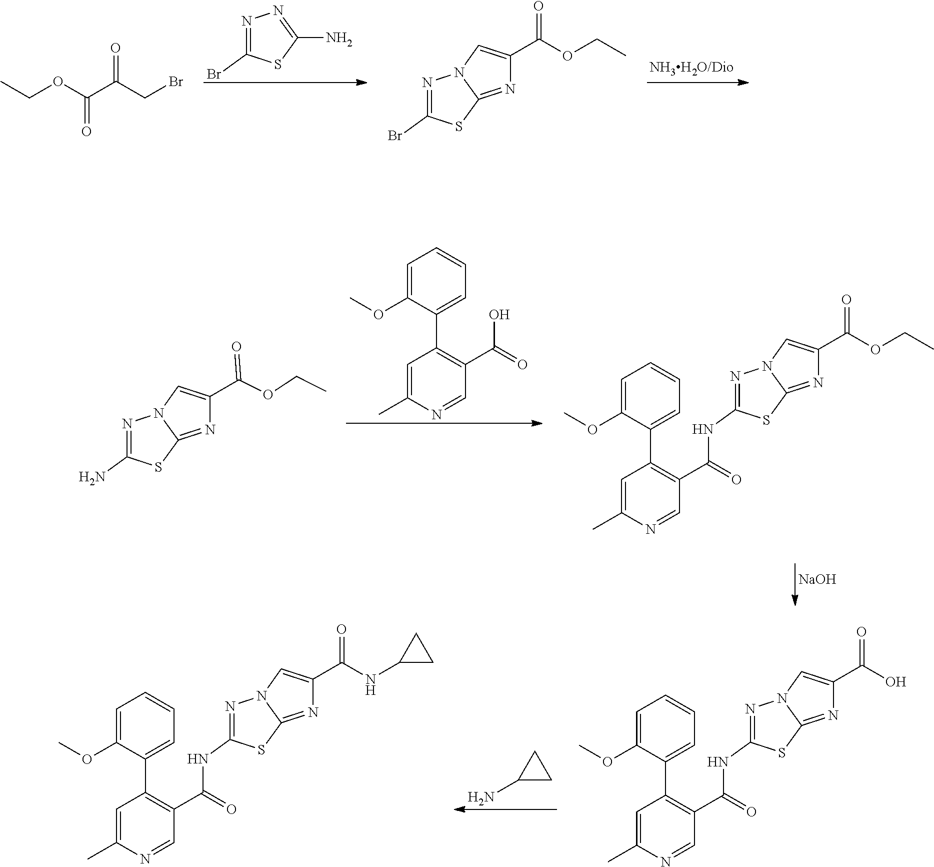

Step 1: A solution of 5-bromo-1,3,4-thiadiazol-2-amine (1.00 g, 5.55 mmol) and ethyl 3-bromo-2-oxopropanoate (1.62 g, 8.33 mmol) in EtOH (12 mL) was heated at 110° C. for 2 h in a sealed tube. After cooling to room temperature, the mixture was concentrated under reduced pressure and the residue purified by silica gel chromatography (Pet. ether/EtOAc=5/1, v/v) to give ethyl 2-bromoimidazo[2,1-b][1,3,4]thiadiazole-6-carboxylate (1.3 g, 17%) as a light yellow solid. LCMS (ESI, m/z): 276, 278 [M+H], ¹H NMR (400 MHz, DMSO-d₆): δ 8.86 (s, 1H), 4.33-4.24 (m, 2H), 1.31-1.25 (m, 3H).

Step 2: A mixture of ethyl 2-bromoimidazo[2,1-b][1,3,4]thiadiazole-6-carboxylate (400 mg, 1.45 mmol), concentrated aqueous NH₄OH (8 mL) and dioxane (8 mL) was heated at 80° C. for 3 h in a sealed tube. After cooling to room temperature, the mixture was concentrated under reduced pressure and the residue purified by silica gel chromatography (DCM/MeOH=50/1, v/v) and prep-TLC (DCM/MeOH=10/1, v/v) to give ethyl 2-aminoimidazo[2,1-b][1,3,4]thiadiazole-6-carboxylate (369 mg, 43%) as a white solid. LCMS (ESI, m/z): 213 [M+H]⁺, ¹H NMR (400 MHz, DMSO-d₆): δ 8.30 (s, 1H), 7.74 (s, 2H), 4.25-4.18 (m, 2H), 1.28-1.24 (m, 3H).

Step 3: A mixture of ethyl 2-aminoimidazo[2,1-b][1,3,4]thiadiazole-6-carboxylate (264 mg, 1.24 mmol), 4-(2-methoxyphenyl)-6-methylnicotinic acid (605 mg, 2.49 mmol), EDCI·HCl (715 mg, 3.73 mmol) and DMAP (456 mg, 3.73 mmol) in dry DMF (5 mL) was heated at 80° C. for 16 h. After cooling to room temperature, the mixture was diluted with water (3 mL), extracted with EtOAc (20 mL×5) and the combined organic phases dried over Na₂SO₄, filtered and concentrated under reduced pressure. The residue was purified by prep-TLC (DCM/MeOH=10/1, v/v) to give ethyl 2-(4-(2-methoxyphenyl)-6-methylnicotinamido)imidazo[2,1-b][1,3,4]thiadiazole-6-carboxylate (381 mg, 70%) as a light yellow solid. LCMS (ESI, m/z): 438 [M+H], ¹H NMR (400 MHz, DMSO-d₆): δ 13.2 (s, 1H), 8.71 (s, 1H), 8.64 (s, 1H), 7.44-7.36 (m, 2H), 7.34 (s, 1H), 7.12-7.05 (m, 1H), 7.00 (d, J=8.2 Hz, 1H), 4.30-4.23 (m, 2H), 3.55 (s, 3H), 2.58 (s, 3H), 1.31-1.27 (m, 3H).

Step 4: A solution of ethyl 2-(4-(2-methoxyphenyl)-6-methylnicotinamido)imidazo[2,1-b][1,3,4]thiadiazole-6-carboxylate (50 mg, 0.11 mmol) and NaOH (14 mg, 0.34 mmol) in THF/water (1 mL/1 mL) was heated at 50° C. for 3.5 h. After cooling to room temperature, the mixture was concentrated under reduced pressure to remove most of the organic solvent and the aqueous residue was adjusted to pH 6-7 with 2 M aq HCl. The resulting precipitate was collected by filtration, washed with water and dried under reduced pressure to give 2-(4-(2-methoxyphenyl)-6-methylnicotinamido)imidazo[2,1-b][1,3,4]thiadiazole-6-carboxylic acid (31 mg, 65%) as a white solid. LCMS (ESI, m/z): 410 [M+H]⁺, ¹H NMR (400 MHz, DMSO-d₆): δ 13.2 (s, 1H), 12.5 (s, 1H), 8.72 (s, 1H), 8.57 (s, 1H), 7.44-7.36 (m, 2H), 7.34 (s, 1H), 7.13-7.06 (m, 1H), 7.00 (d, J=8.0 Hz, 1H), 3.55 (s, 3H), 2.59 (s, 3H).

Step 5: A solution of 2-(4-(2-methoxyphenyl)-6-methylnicotinamido)imidazo[2,1-b][1,3,4]thiadiazole-6-carboxylic acid (30 mg, 0.07 mmol), cyclopropanamine (8 mg, 0.14 mmol), HATU (42 mg, 0.11 mmol) and DIPEA (28 mg, 0.22 mmol) in dry DMF (1 mL) was stirred at room temperature for 16 h. The mixture was purified directly by prep-HPLC to give N-cyclopropyl-2-(4-(2-methoxyphenyl)-6-methylnicotinamido)imidazo[2,1-b][1,3,4]thiadiazole-6-carboxamide (17.6 mg, 53%) as a white solid. LCMS (ESI, m/z): 449 [M+H]⁺, ¹H NMR (400 MHz, DMSO-d₆): δ 13.2 (s, 1H), 8.72 (s, 1H), 8.37 (s, 1H), 8.14 (d, J=4.8 Hz, 1H), 7.42-7.35 (m, 2H), 7.32 (s, 1H), 7.12-7.05 (m, 1H), 7.00 (d, J=8.2 Hz, 1H), 3.55 (s, 3H), 2.86-2.79 (m, 1H), 2.58 (s, 3H), 0.67-0.62 (m, 2H), 0.62-0.58 (m, 2H).

Table L: The following examples were prepared using a similar procedure to that described for Example 1

TABLE L
The following examples were prepared using a similar procedure to that described for
Example 1

| Ex. | | | |
|---|---|---|---|
| No. | Structure | Compound Name | Characterization Data |
| 2 | 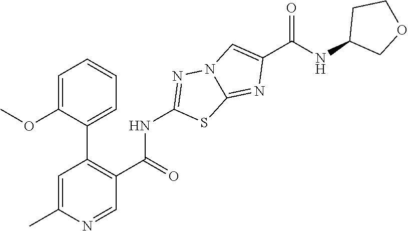 [SMILES: COc1ccccc1-c1cc(C)ncc1C(=O)Nc1nn2cc(C(=O)N[C@H]3CCOC3)nc2s1] | (S)-2-(4-(2- Methoxyphenyl)-6- methylnicotinamido)-N- (tetrahydrofuran-3- yl)imidazo[2,1- b][1,3,4]thiadiazole-6- carboxamide | LCMS (m/z): 479 [M + H]⁺. ¹H NMR (400 MHz, DMSO-d₆) δ 13.1 (s, 1H), 8.73 (s, 1H), 8.36 (s, 1H), 8.00 (d, J = 7.2 Hz, 1H), 7.43-7.34 (m, 2H), 7.31 (s, 1H), 7.08 (t, J = 7.4 Hz, 1H), 7.00 (d, J = 8.2 Hz, 1H), 4.49-4.40 (m, 1H), 3.89- 3.80 (m, 2H), 3.77-3.67 (m, 1H), 3.59-3.54 (m, 4H), 2.59 (s, 3H), 2.18-2.08 (m, 1H), 1.99-1.89 (m, 1H) |
| 3 | 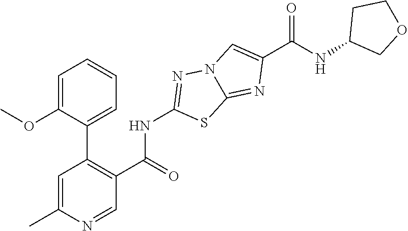 [SMILES: COc1ccccc1-c1cc(C)ncc1C(=O)Nc1nn2cc(C(=O)N[C@@H]3CCOC3)nc2s1] | (R)-2-(4-(2- Methoxyphenyl)-6- methylnicotinamido)-N- (tetrahydrofuran-3- yl)imidazo[2,1- b][1,3,4]thiadiazole-6- carboxamide | LCMS (m/z): 479 [M + H]⁺. ¹H NMR (400 MHz, DMSO-d₆) δ 13.2 (s, 1H), 8.72 (s, 1H), 8.43 (s, 1H), 8.18 (d, J = 7.2 Hz, 1H), 7.45-7.34 (m, 2H), 7.34 (s, 1H), 7.09 (t, J = 7.2 Hz, 1H), 7.00 (d, J = 8.2 Hz, 1H), 4.49-4.38 (m, 1H), 3.89- 3.78 (m, 2H), 3.76-3.65 (m, 1H), 3.59-3.51 (m, 4H), 2.58 (s, 3H), 2.18-2.05 (m, 1H), 2.00-1.87 (m, 1H) |
| 4 | 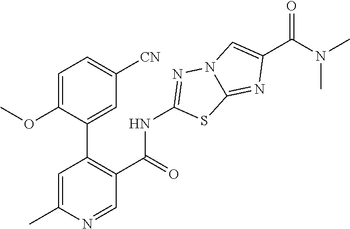 | 2-(4-(5-Cyano-2- methoxyphenyl)-6- methylnicotinamido)- N,N-dimethylimidazo[2,1- b][1,3,4]thiadiazole-6- carboxamide | LCMS (m/z): 462 [M + H]⁺. ¹H NMR (400 MHz, DMSO-d₆) δ 13.2 (s, 1H), 8.78 (s, 1H), 8.35 (s, 1H), 7.96-7.87 (m, 2H), 7.43 (s, 1H), 7.20 (d, J = 8.6 Hz, 1H), 3.64 (s, 3H), 3.39 (s, 3H), 2.97 (s, 3H), 2.59 (s, 3H) |
| 5 | 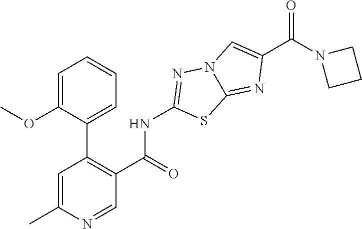 [SMILES: COc1ccccc1-c1cc(C)ncc1C(=O)Nc1nn2cc(C(=O)N3CCC3)nc2s1] | N-(6-(Azetidine-1- carbonyl)imidazo[2,1- b][1,3,4]thiadiazol-2-yl)- 4-(2-methoxyphenyl)-6- methylnicotinamide | LCMS (m/z): 449 [M + H]⁺. ¹H NMR (400 MHz, DMSO-d₆) δ 13.2 (s, 1H), 8.71 (s, 1H), 8.41 (s, 1H), 7.44-7.37 (m, 2H), 7.34 (s, 1H), 7.09 (t, J = 7.6 Hz, 1H), 7.00 (d, J = 8.2 Hz, 1H), 4.55 (t, J = 7.6 Hz, 2H), 4.01 (t, J = 7.8 Hz, 2H), 3.55 (s, 3H), 2.58 (s, 3H), 2.27 (t, J = 7.8 Hz, 2H) |
| 6 | 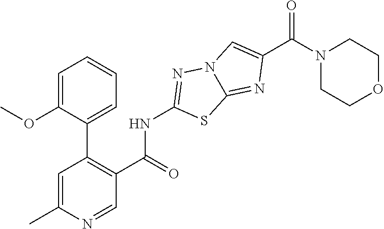 [SMILES: COc1ccccc1-c1cc(C)ncc1C(=O)Nc1nn2cc(C(=O)N3CCOCC3)nc2s1] | 4-(2-Methoxyphenyl)-6- methyl-N-(6-(morpholine- 4-carbonyl)imidazo[2,1- b][1,3,4]thiadiazol-2- yl)nicotinamide | LCMS (m/z): 479 [M + H]⁺. ¹H NMR (400 MHz, DMSO-d₆) δ 13.0 (s, 1H), 8.72 (s, 1H), 8.35 (s, 1H), 7.43-7.35 (m, 2H), 7.32 (s, 1H), 7.08 (t, J = 7.4 Hz, 1H), 7.00 (d, J = 8.2 Hz, 1H), 3.90 (br s, 4H), 3.63 (t, J = 4.6 Hz, 4H), 3.57 (s, 3H), 2.59 (s, 3H) |
| 7 | 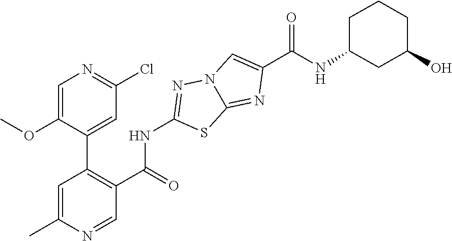 [SMILES: COc1cnc(Cl)cc1-c1cc(C)ncc1C(=O)Nc1nn2cc(C(=O)N[C@@H]3CCC[C@@H](O)C3)nc2s1] | 2-(2′-chloro-5′-methoxy- 6-methyl-[4,4′- bipyridine]-3- carboxamido)-N- ((1R,3R)-3- hydroxycyclohexyl)imida zo[2,1- b][1,3,4]thiadiazole-6- carboxamide | LCMS (m/z): 542 [M + H]⁺. ¹H NMR (400 MHz, DMSO-d₆) δ 13.37 (s, 1H), 8.90 (s, 1H), 8.34 (s, 1H), 8.17-8.13 (m, 1H), 7.71 (s, 1H), 7.52-7.40 (m, 2H), 4.48-4.43 (m, 1H), 4.18-4.15 (m, 1H), 3.94 (d, J = 5.6 Hz, 1H), 3.66 (s, 3H), 2.58 (s, 3H), 1.70 (s, 3H), 1.63- 1.52 (m, 1H), 1.48-1.33 (m, 4H) |
| 8 | 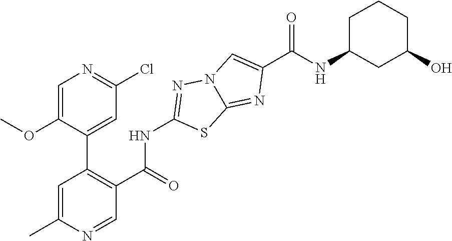 [SMILES: COc1cnc(Cl)cc1-c1cc(C)ncc1C(=O)Nc1nn2cc(C(=O)N[C@H]3CCC[C@@H](O)C3)nc2s1] | 2-(2′-chloro-5′-methoxy- 6-methyl-[4,4′- bipyridine]-3- carboxamido)-N- ((1S,3R)-3- hydroxycyclohexyl) imidazo[2,1- b][1,3,4]thiadiazole-6- carboxamide | LCMS (m/z): 542 [M + H]⁺. ¹H NMR (400 MHz, DMSO-d₆) δ 8.95 (s, 1H), 8.19-8.16 (m, 2H), 7.67 (s, 1H), 7.45 (s, 1H), 7.30 (s, 1H), 4.47 (d, J = 3.3 Hz, 1H), 4.18-4.14 (m, 1H), 3.94 (s, 1H), 3.66 (s, 4H), 2.56 (s, 3H), 1.74-1.68 (m, 3H), 1.62-1.41 (m, 1H), 1.41- 1.27 (m, 2H), 1.28-1.22 (m, 2H) |
| 9 | 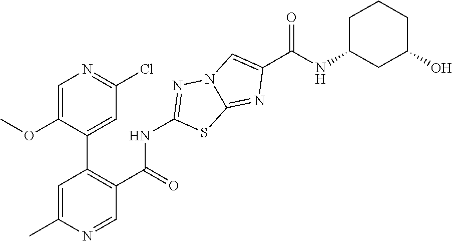 [SMILES: COc1cnc(Cl)cc1-c1cc(C)ncc1C(=O)Nc1nn2cc(C(=O)N[C@@H]3CCC[C@H](O)C3)nc2s1] | 2-(2′-chloro-5′-methoxy- 6-methyl-[4,4′- bipyridine]-3- carboxamido)-N- ((1R,3S)-3- hydroxycyclohexyl) imidazo[2,1- b][1,3,4]thiadiazole-6- carboxamide | LCMS (m/z): 542 [M + H]⁺. ¹H NMR (400 MHz, DMSO-d₆) δ 13.37 (s, 1H), 8.89 (s, 1H), 8.33 (s, 1H), 8.23-8.10 (m, 1H), 8.05 (s, 1H), 7.51 (s, 1H), 7.43 (s, 1H), 4.69 (d, J = 4.2 Hz, 1H), 3.81 (s, 1H), 3.66 (d, J = 2.6 Hz, 3H), 3.51 (s, 1H), 2.59 (d, J = 4.1 Hz, 3H), 1.92- 1.89 (m, 1H), 1.81-1.58 (m, 3H), 1.43-1.20 (m, 3H), 1.20- 1.02 (m, 1H) |
| 10 | 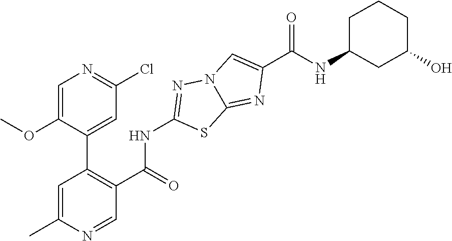 [SMILES: COc1cnc(Cl)cc1-c1cc(C)ncc1C(=O)Nc1nn2cc(C(=O)N[C@H]3CCC[C@H](O)C3)nc2s1] | 2-(2′-chloro-5′-methoxy- 6-methyl-[4,4′- bipyridine]-3- carboxamido)-N-((1S,3S)- 3-hydroxycyclohexyl) imidazo[2,1- b][1,3,4]thiadiazole-6- carboxamide | LCMS (m/z): 542 [M + H]⁺. ¹H NMR (300 MHz, DMSO-d₆) δ 13.37 (s, 1H), 8.85 (s, 1H), 8.44 (s, 1H), 8.20 (s, 1H), 7.74 (d, J = 8.7 Hz, 1H), 7.59 (s, 1H), 7.49 (s, 1H), 4.17 (s, 1H), 3.94 (s, 1H), 3.67 (s, 3H), 2.61 (s, 3H), 1.69 (s, 2H), 1.44- 1.31 (m, 6H) |
| 11 | 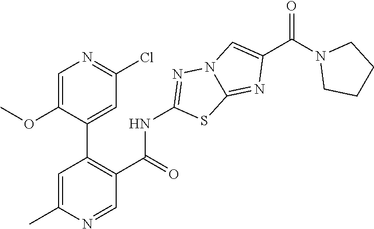 [SMILES: COc1cnc(Cl)cc1-c1cc(C)ncc1C(=O)Nc1nn2cc(C(=O)N3CCCC3)nc2s1] | 2′-chloro-5′-methoxy-6- methyl-N-(6-(pyrrolidine- 1-carbonyl)imidazo[2,1- b][1,3,4]thiadiazol-2-yl)- [4,4′-bipyridine]-3- carboxamide | LCMS (m/z): 498 [M + H]⁺. ¹H NMR (300 MHz, DMSO-d₆) δ 13.35 (s, 1H), 8.86 (s, 1H), 8.39 (s, 1H), 8.19 (s, 1H), 7.57 (s, 1H), 7.46 (s, 1H), 3.94 (t, J = 6.6 Hz, 2H), 3.67 (s, 3H), 3.48 (t, J = 6.8 Hz, 2H), 2.60 (s, 3H), 1.87-1.83 (m, 4H) |
| 12 | 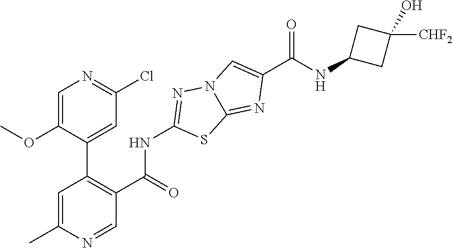 | 2-(2′-chloro-5′-methoxy- 6-methyl-[4,4′- bipyridine]-3- carboxamido)-N-((1r,3r)- 3-hydroxy-3- (trifluoromethyl) cyclobutyl)imidazo[2,1- b][1,3,4]thiadiazole-6- carboxamide | LCMS (m/z): 582 [M + H]⁺. ¹H NMR (300 MHz, DMSO-d₆) δ 13.41 (s, 1H), 8.87 (s, 1H), 8.68 (d, J = 7.9 Hz, 1H), 8.43 (s, 1H), 8.20 (s, 1H), 7.58- 7.47 (m, 2H), 6.44 (s, 1H), 4.67-4.62 (m, 1H), 3.67 (s, 3H), 2.61 (s, 3H), 2.47 (d, J = 4.3 Hz, 1H), 2.37-2.21 (m, 2H), 1.24 (s, 1H) |
| 13 | 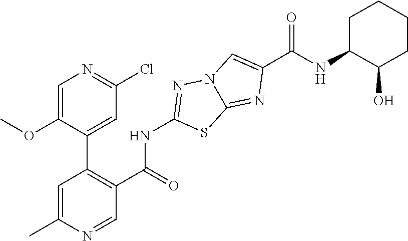 [SMILES: COc1cnc(Cl)cc1-c1cc(C)ncc1C(=O)Nc1nn2cc(C(=O)N[C@H]3CCCC[C@H]3O)nc2s1] | 2-(2′-chloro-5′-methoxy- 6-methyl-[4,4′- bipyridine]-3- carboxamido)-N- ((1S,2R)-2- hydroxycyclohexyl) imidazo[2,1- b][1,3,4]thiadiazole-6- carboxamide | LCMS (m/z): 542 [M + H]⁺. ¹H NMR (400 MHz, DMSO-d₆) δ 13.35 (s, 1H), 8.95 (s, 1H), 8.14 (d, J = 7.0 Hz, 2H), 7.54- 7.26 (m, 2H), 4.90 (d, J = 3.9 Hz, 1H), 3.76 (s, 2H), 3.65 (s, 3H), 2.56 (s, 3H), 1.73-1.69 (m, 1H), 1.60-1.45 (m, 5H), 1.33-1.29 (m, 2H) |
| 14 | 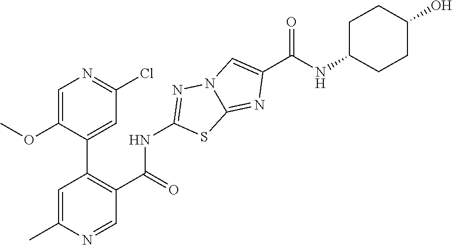 [SMILES: COc1cnc(Cl)cc1-c1cc(C)ncc1C(=O)Nc1nn2cc(C(=O)N[C@H]3CC[C@@H](O)CC3)nc2s1] | 2-(2′-chloro-5′-methoxy- 6-methyl-[4,4′- bipyridine]-3- carboxamido)-N-((1s,4s)- 4-hydroxycyclohexyl) imidazo[2,1- b][1,3,4]thiadiazole-6- carboxamide | LCMS (m/z): 542 [M + H]⁺. ¹H NMR (400 MHz, Chloroform- d) δ 8.91 (s, 1H), 8.18 (s, 1H), 8.10 (s, 1H), 7.28 (s, 1H) 7.26 (s, 3H), 7.19 (s, 1H), 3.96 (s, 2H), 3.83 (d, J = 1.3 Hz, 3H), 2.72 (s, 3H), 1.76 (s, 8H), 1.25 (s, 2H) |
| 15 | 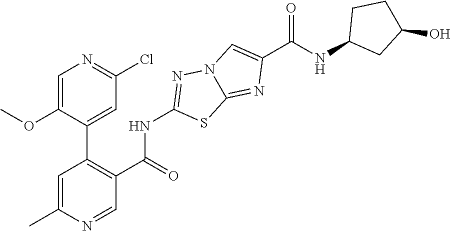 [SMILES: COc1cnc(Cl)cc1-c1cc(C)ncc1C(=O)Nc1nn2cc(C(=O)N[C@H]3CC[C@@H](O)C3)nc2s1] | 2-(2′-chloro-5′-methoxy- 6-methyl-[4,4′- bipyridine]-3- carboxamido)-N- ((1S,3R)-3- hydroxycyclopentyl) imidazo[2,1- b][1,3,4]thiadiazole-6- carboxamide | LCMS (m/z): 528 [M + H]⁺. ¹H NMR (300 MHz, DMSO-d₆) δ 9.02 (s, 1H), 8.11 (s, 1H), 7.94 (s, 2H), 7.31 (s, 1H), 7.13 (s, 1H), 4.77 (d, J = 3.8 Hz, 1H), 4.31-4.28 (m, 1H), 4.15 (s, 1H), 3.34 (s, 3H), 2.50 (s, 3H), 1.96-1.94 (m, 1H), 1.90- 1.88 (m, 1H), 1.70-1.65 (m, 3H), 1.56-1.51 (m, 1H) |
| 16 | 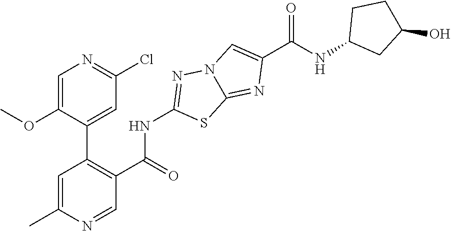 [SMILES: COc1cnc(Cl)cc1-c1cc(C)ncc1C(=O)Nc1nn2cc(C(=O)N[C@@H]3CC[C@@H](O)C3)nc2s1] | 2-(2′-chloro-5′-methoxy- 6-methyl-[4,4′- bipyridine]-3- carboxamido)-N- ((1R,3R)-3- hydroxycyclopentyl) imidazo[2,1- b][1,3,4]thiadiazole-6- carboxamide | LCMS (m/z): 528 [M + H]⁺. ¹H NMR (400 MHz, DMSO-d₆) δ 13.40 (s, 1H), 8.92 (s, 1H), 8.21-8.16 (m, 2H), 7.93- 7.91 (m, 1H), 7.47 (s, 1H), 7.33 (s, 1H), 4.49-4.44 (m, 2H), 4.18 (s, 1H), 3.65 (s, 3H), 2.57 (s, 3H), 2.01-1.91 (m, 2H), 1.90-1.81 (m, 1H), 1.79 -1.65 (m, 1H), 1.50-1.38 (m, 2H) |
| 17 | 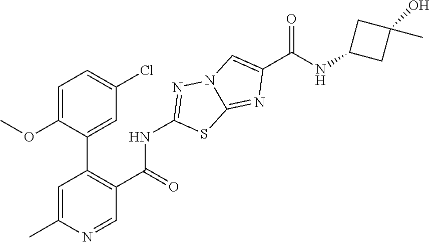 | 2-(4-(5-chloro-2- methoxyphenyl)-6- methylnicotinamido)-N- ((1s,3s)-3-hydroxy-3- methylcyclobutyl)imidazo [2,1-b][1,3,4]thiadiazole- 6-carboxamide | LCMS (m/z): 528 [M + H]⁺. ¹H NMR (400 MHz, Methanol-d₄) δ 8.88 (s, 1H), 8.09 (s, 1H), 8.01 (s, 1H), 7.40 (s, 1H), 7.27 (s, 1H), 4.10 (s, 1H), 3.71 (s, 3H), 2.62 (s, 3H), 2.52-2.47 (m, 2H), 2.23-2.11 (m, 2H), 1.37 (s, 3H) |
| 18 | 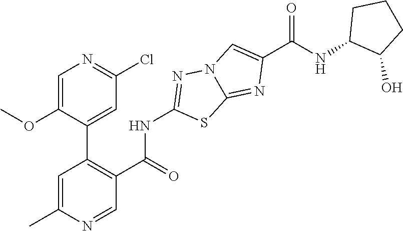 [SMILES: COc1cnc(Cl)cc1-c1cc(C)ncc1C(=O)Nc1nn2cc(C(=O)N[C@@H]3CCC[C@@H]3O)nc2s1] | 2-(2′-chloro-5′-methoxy- 6-methyl-[4,4′- bipyridine]-3- carboxamido)-N- ((1R,2S)-2- hydroxycyclopentyl) imidazo[2,1- b][1,3,4]thiadiazole-6- carboxamide | LCMS (m/z): 528 [M + H]⁺. ¹H NMR (400 MHz, DMSO-d₆) δ 13.35 (s, 1H), 8.88 (s, 1H), 8.37 (s, 1H), 8.18 (s, 1H), 7.64 (d, J = 7.6 Hz, 1H), 7.54 (s, 1H), 7.42 (s, 1H), 5.05 (d, J = 4.2 Hz, 1H), 3.99 (d, J = 5.9 Hz, 2H), 3.67 (s, 3H), 2.59 (s, 3H), 1.98-1.69 (m, 3H), 1.62- 1.46 (m, 3H) |
| 19 | 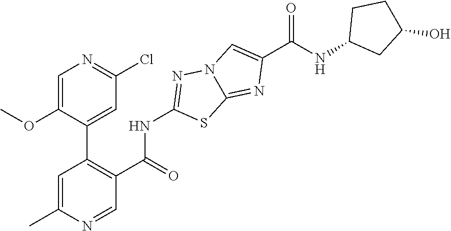 [SMILES: COc1cnc(Cl)cc1-c1cc(C)ncc1C(=O)Nc1nn2cc(C(=O)N[C@@H]3CC[C@H](O)C3)nc2s1] | 2-(2′-chloro-5′-methoxy- 6-methyl-[4,4′- bipyridine]-3- carboxamido)-N- ((1R,3S)-3- hydroxycyclopentyl) imidazo[2,1- b][1,3,4]thiadiazole-6- carboxamide | LCMS (m/z): 528 [M + H]⁺. ¹H NMR (300 MHz, DMSO-d₆) δ 13.36 (s, 1H), 8.86 (s, 1H), 8.41 (s, 1H), 8.19 (s, 1H), 8.04 (d, J = 8.7 Hz, 1H), 7.58 (s, 1H), 7.47 (s, 1H), 4.80 (d, J = 3.8 Hz, 1H), 4.35-3.43 (m, 1H), 4.17 (s, 1H), 3.66 (s, 3H), 2.60 (s, 3H), 2.07-1.79 (m, 2H), 1.78-1.60 (m, 3H), 1.55- 1.52 (m, 1H) |
| 20 | 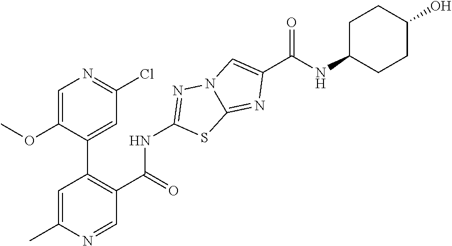 [SMILES: COc1cnc(Cl)cc1-c1cc(C)ncc1C(=O)Nc1nn2cc(C(=O)N[C@H]3CC[C@H](O)CC3)nc2s1] | 2-(2′-chloro-5′-methoxy- 6-methyl-[4,4′- bipyridine]-3- carboxamido)-N-((1r,4r)- 4-hydroxycyclohexyl) imidazo[2,1- b][1,3,4]thiadiazole-6- carboxamide | LCMS (m/z): 542 [M + H]⁺. ¹H NMR (400 MHz, DMSO-d₆) δ 9.04 (s, 1H), 8.12 (s, 2H), 7.93 (s, 1H), 7.66 (s, 1H), 7.31 (s, 1H), 7.12 (s, 1H), 4.57 (s, 1H), 3.63 (s, 3H), 1.83-1.76 (m, 6H), 1.53-1.06 (m, 6H) |
| 21 | 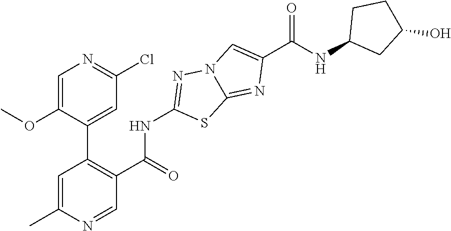 [SMILES: COc1cnc(Cl)cc1-c1cc(C)ncc1C(=O)Nc1nn2cc(C(=O)N[C@H]3CC[C@H](O)C3)nc2s1] | 2-(2′-chloro-5′-methoxy- 6-methyl-[4,4′- bipyridine]-3- carboxamido)-N-((1S,3S)- 3-hydroxycyclopentyl) imidazo[2,1- b][1,3,4]thiadiazole-6- carboxamide | LCMS (m/z): 528 [M + H]⁺. ¹H NMR (400 MHz, DMSO-d₆) δ 13.38 (s, 1H), 8.92 (s, 1H), 8.31-8.13 (m, 2H), 7.93 (d, J = 8.5 Hz, 1H), 7.49 (s, 1H), 7.36 (s, 1H), 4.48 (d, J = 3.7 Hz, 1H), 4.43 (q, J = 7.9 Hz, 1H), 4.19 (s, 1H), 3.66 (d, J = 1.8 Hz, 3H), 2.57 (d, J = 4.2 Hz, 3H), 2.02-1.87 (m, 2H), 1.81-1.78 (m, 1H), 1.71-1.67 (m, 1H), 1.53-1.39 (m, 2H) |
| 22 | 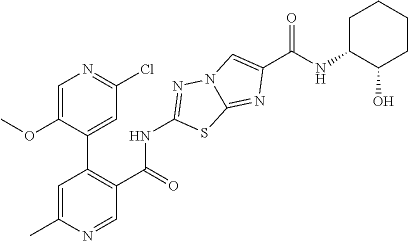 [SMILES: COc1cnc(Cl)cc1-c1cc(C)ncc1C(=O)Nc1nn2cc(C(=O)N[C@@H]3CCCC[C@@H]3O)nc2s1] | 2-(2′-chloro-5′-methoxy- 6-methyl-[4,4′- bipyridine]-3- carboxamido)-N- ((1R,2S)-2- hydroxycyclohexyl) imidazo[2,1- b][1,3,4]thiadiazole-6- carboxamide | LCMS (m/z): 542 [M + H]⁺. ¹H NMR (400 MHz, DMSO-d₆) δ 13.35 (s, 1H), 8.86 (s, 1H), 8.43 (s, 1H), 8.19 (s, 1H), 7.57 (d, J = 7.5 Hz, 2H), 7.46 (s, 1H), 5.24-4.69 (m, 1H), 3.78 (s, 1H), 3.67 (d, J = 1.5 Hz, 3H), 2.60 (s, 3H), 1.74-1.71 (m, 1H), 1.56-1.53 (m, 5H), 1.40-1.15 (m, 3H) |
| 23 | 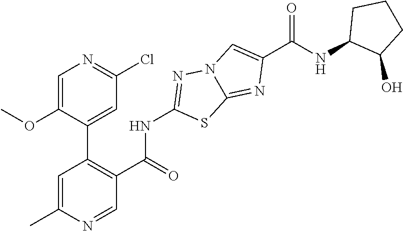 [SMILES: COc1cnc(Cl)cc1-c1cc(C)ncc1C(=O)Nc1nn2cc(C(=O)N[C@H]3CCC[C@H]3O)nc2s1] | 2-(2′-chloro-5′-methoxy- 6-methyl-[4,4′- bipyridine]-3- carboxamido)-N- ((1S,2R)-2- hydroxycyclopentyl) imidazo[2,1- b][1,3,4]thiadiazole-6- carboxamide | LCMS (m/z): 528 [M + H]⁺. ¹H NMR (400 MHz, DMSO-d₆) δ 13.35 (s, 1H), 8.86 (s, 1H), 8.43 (s, 1H), 8.19 (s, 1H), 7.64 (d, J = 7.6 Hz, 1H), 7.57 (s, 1H), 7.46 (s, 1H), 5.05 (d, J = 4.0 Hz, 1H), 4.00 (d, J = 5.9 Hz, 2H), 3.67 (s, 3H), 2.60 (s, 3H), 1.95-1.64 (m, 3H), 1.65- 1.47 (m, 3H) |
| 24 | 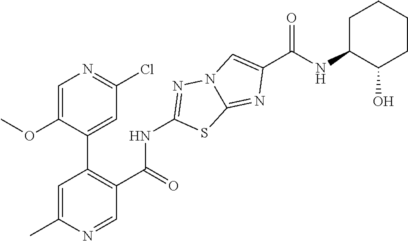 [SMILES: COc1cnc(Cl)cc1-c1cc(C)ncc1C(=O)Nc1nn2cc(C(=O)N[C@H]3CCCC[C@@H]3O)nc2s1] | 2-(2′-chloro-5′-methoxy- 6-methyl-[4,4′- bipyridine]-3- carboxamido)-N-((1S,2S)- 2-hydroxycyclohexyl) imidazo[2,1- b][1,3,4]thiadiazole-6- carboxamide | LCMS (m/z): 542 [M + H]⁺. ¹H NMR (300 MHz, DMSO-d₆) δ 13.37 (s, 1H), 8.87 (s, 1H), 8.42 (s, 1H), 8.19 (s, 1H), 7.72 (d, J = 7.6 Hz, 1H), 7.58 (s, 1H), 7.47 (s, 1H), 4.65 (s, 1H), 3.67 (s, 3H), 3.52 (s, 2H), 2.60 (s, 3H), 1.88 (s, 2H), 1.63 (s, 2H), 1.22 (d, J = 7.9 Hz, 4H) |
| 25 | 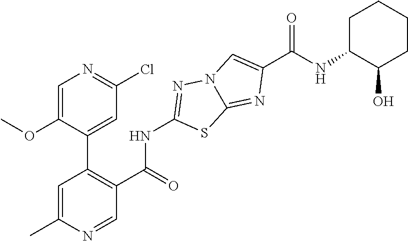 [SMILES: COc1cnc(Cl)cc1-c1cc(C)ncc1C(=O)Nc1nn2cc(C(=O)N[C@@H]3CCCC[C@H]3O)nc2s1] | 2-(2′-chloro-5′-methoxy- 6-methyl-[4,4′- bipyridine]-3- carboxamido)-N- ((1R,2R)-2- hydroxycyclohexyl) imidazo[2,1- b][1,3,4]thiadiazole-6- carboxamide | LCMS (m/z): 542 [M + H]⁺. ¹H NMR (400 MHz, DMSO-d₆) δ 9.01 (s, 1H), 8.13 (s, 1H), 7.99 (s, 1H), 7.61 (s, 1H), 7.34 (s, 1H), 7.16 (s, 1H), 4.67 (d, J = 5.3 Hz, 1H), 3.65 (s, 3H), 3.51 (d, J = 4.2 Hz, 2H), 2.53 (s, 3H), 1.89-1.86 (m, 2H), 1.64- 1.59 (m, 2H), 1.22 (t, J = 6.1 Hz, 4H) |
| 26 | 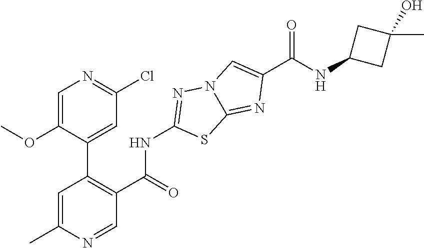 | 2-(2′-chloro-5′-methoxy- 6-methyl-[4,4′- bipyridine]-3- carboxamido)-N-((1r,3r)- 3-hydroxy-3- methylcyclobutyl)imidazo [2,1-b][1,3,4]thiadiazole- 6-carboxamide | LCMS (m/z): 528 [M + H]⁺. ¹H NMR (400 MHz, Methanol-d₄) δ 8.75 (s, 1H), 8.00 (s, 1H), 7.94 (s, 1H), 7.34 (s, 1H), 7.23 (s, 1H), 4.10-3.93 (m, 1H), 3.62 (s, 3H), 2.54 (s, 3H), 2.44- 2.33 (m, 2H), 2.12-2.00 (m, 2H), 1.29 (s, 3H) |
| 27 | 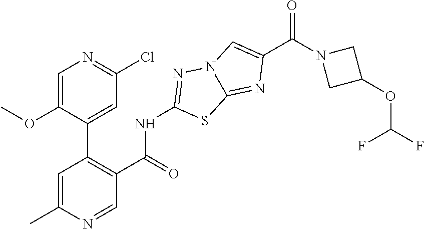 [SMILES: COc1cnc(Cl)cc1-c1cc(C)ncc1C(=O)Nc1nn2cc(C(=O)N3CC(OC(F)F)C3)nc2s1] | 2′-chloro-N-(6-(3- (difluoromethoxy) azetidine-1-carbonyl) imidazo[2,1- b][1,3,4]thiadiazol-2-y1)- 5′-methoxy-6-methyl- [4,4′-bipyridine]-3- carboxamide | LCMS (m/z): 550 [M + H]⁺. ¹H NMR (300 MHz, DMSO-d₆) δ 8.88 (s, 1H), 8.36 (s, 1H), 8.15 (d, J = 8.3 Hz, 1H), 7.51 (s, 1H), 7.38 (s, 1H), 6.84-6.72 (m, 1H), 5.03 (t, J = 4.7 Hz, 1H), 4.88 (t, J = 8.7 Hz, 1H), 4.52-4.46 (m, 1H), 4.38- 4.33 (m, 1H), 3.99-3.93 (m, 1H), 3.65 (s, 3H), 2.58 (s, 3H) |
| 28 | 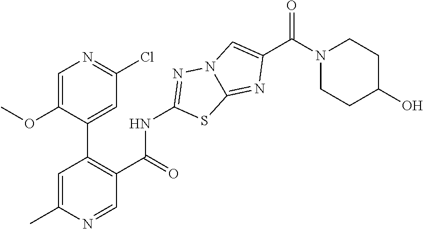 [SMILES: COc1cnc(Cl)cc1-c1cc(C)ncc1C(=O)Nc1nn2cc(C(=O)N3CCC(O)CC3)nc2s1] | 2′-chloro-N-(6-(4- hydroxypiperidine-1- carbonyl)imidazo[2,1- b][1,3,4]thiadiazol-2-yl)- 5′-methoxy-6-methyl- [4,4′-bipyridine]-3- carboxamide | LCMS (m/z): 528 [M + H]⁺. ¹H NMR (400 MHz, DMSO-d₆) δ 13.32 (s, 1H), 8.85 (s, 1H), 8.35 (s, 1H), 8.19 (s, 1H), 7.58 (s, 1H), 7.46 (s, 1H), 4.75 (s, 1H), 4.60-4.57 (m, 1H), 4.06- 4.03 (m, 1H), 3.73 (m, 1H), 3.67 (s, 3H), 2.60 (s, 3H), 1.83- 1.72 (m, 2H), 1.46-1.28 (m, 2H), 1.24 (s, 1H) |
| 29 | 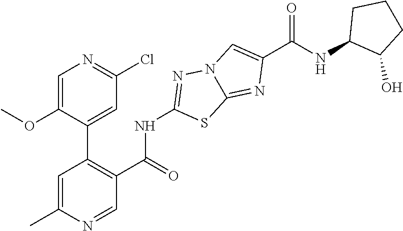 [SMILES: COc1cnc(Cl)cc1-c1cc(C)ncc1C(=O)Nc1nn2cc(C(=O)N[C@H]3CCC[C@@H]3O)nc2s1] | 2-(2′-chloro-5′-methoxy- 6-methyl-[4,4′- bipyridine]-3- carboxamido)-N-((1S,2S)- 2-hydroxycyclopentyl) imidazo[2,1- b][1,3,4]thiadiazole-6- carboxamide | LCMS (m/z): 528 [M + H]⁺. ¹H NMR (400 MHz, DMSO-d₆) δ 13.39 (s, 1H), 8.96 (s, 1H), 8.15 (s, 1H), 7.81 (s, 1H), 7.44 (s, 2H), 4.79 (d, J = 4.0 Hz, 1H), 3.94 (d, J = 7.6 Hz, 2H), 3.65 (s, 3H), 2.56 (s, 3H), 2.01- 1.92 (m, 1H), 1.85-1.81 (m, 1H), 1.67-1.58 (m, 2H), 1.49- 1.46 (m, 2H) |
| 30 | 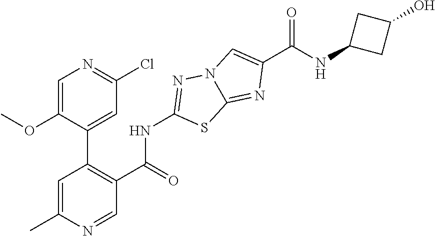 [SMILES: COc1cnc(Cl)cc1-c1cc(C)ncc1C(=O)Nc1nn2cc(C(=O)N[C@H]3C[C@H](O)C3)nc2s1] | 2-(2′-chloro-5′-methoxy- 6-methyl-[4,4′- bipyridine]-3- carboxamido)-N-((1r,3r)- 3-hydroxycyclobutyl) imidazo[2,1-b] [1,3,4]thiadiazole- 6-carboxamide | LCMS (m/z): 514 [M + H]⁺. ¹H NMR (300 MHz, DMSO-d₆) δ 13.4 (s, 1H), 8.86 (s, 1H), 8.38- 8.35 (m, 2H), 8.19 (s, 1H), 7.58 (s, 1H), 7.46 (s, 1H), 5.01 (s, 1H), 4.59-4.34 (m, 1H), 4.29 (s, 1H), 3.67 (s, 3H), 2.60 (s, 3H), 2.36-2.22 (m, 2H), 2.16-2.03 (m, 2H) |
| 31 | 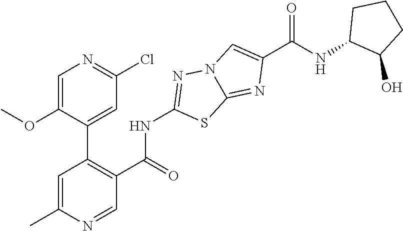 [SMILES: COc1cnc(Cl)cc1-c1cc(C)ncc1C(=O)Nc1nn2cc(C(=O)N[C@@H]3CCC[C@H]3O)nc2s1] | 2-(2′-chloro-5′-methoxy- 6-methyl-[4,4′- bipyridine]-3- carboxamido)-N- ((1R,2R)-2- hydroxycyclopentyl) imidazo[2,1- b][1,3,4]thiadiazole-6- carboxamide | LCMS (m/z): 528 [M + H]⁺. ¹H NMR (400 MHz, DMSO-d₆) δ 13.36 (s, 1H), 8 8.91 (s, 1H), 8.26-8.17 (s, 2H), 7.83 (d, J = 7.1 Hz, 1H), 7.50 (s, 1H), 7.37 (s, 1H), 4.78 (d, J = 3.9 Hz, 1H), 4.01-3.88 (m, 2H), 3.66 (s, 3H), 2.58 (s, 3H), 1.97- 1.79 (m, 2H), 1.63 (d, J = 7.6 Hz, 2H), 1.47-1.15 (m, 5H) |
| 32 | 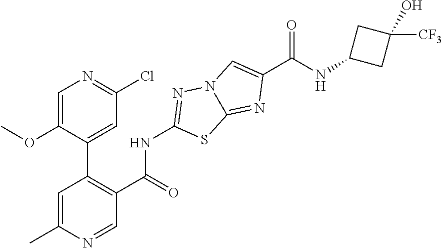 [SMILES: COc1cnc(Cl)cc1-c1cc(C)ncc1C(=O)Nc1nn2cc(C(=O)N[C@H]3C[C@](O)(C(F)(F)F)C3)nc2s1] | 2-(2′-chloro-5′-methoxy- 6-methyl-[4,4′- bipyridine]-3- carboxamido)-N-((1s,3s)- 3-hydroxy-3- (trifluoromethyl) cyclobutyl)imidazo[2,1- b][1,3,4]thiadiazole-6- carboxamide | LCMS (m/z): 582 [M + H]⁺. ¹H NMR (400 MHz, DMSO-d₆) δ 13.40 (s, 1H), 8.86 (s, 1H), 8.58 (d, J = 7.9 Hz, 1H), 8.44 (s, 1H), 8.20 (s, 1H), 7.58 (s, 1H), 7.47 (s, 1H), 6.60 (s, 1H), 4.14 (q, J = 8.1 Hz, 1H), 3.67 (s, 3H), 2.84-2.70 (m, 2H), 2.61 (s, 3H), 2.39-2.36 (m, 2H) |
| 33 | 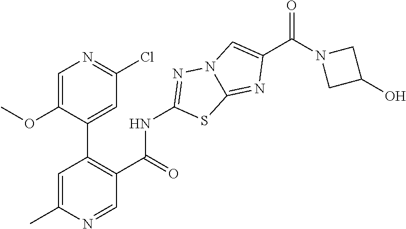 [SMILES: COc1cnc(Cl)cc1-c1cc(C)ncc1C(=O)Nc1nn2cc(C(=O)N3CC(O)C3)nc2s1] | 2′-chloro-N-(6-(3- hydroxyazetidine-1- carbonyl)imidazo[2,1- b][1,3,4]thiadiazol-2-yl)- 5′-methoxy-6-methyl- [4,4′-bipyridine]-3- carboxamide | LCMS (m/z): 500 [M + H]⁺. ¹H NMR (400 MHz, DMSO-d₆) δ 9.01 (s, 1H), 8.09 (s, 1H), 7.93 (s, 1H), 7.29 (d, J = 7.6 Hz, 1H), 7.15 (s, 1H), 5.66 (d, J = 4.0 Hz, 1H), 4.67 (m, 2H), 4.20 (s, 2H), 3.72 (s, 1H), 3.71 (s, 3H), 2.67 (s, 3H) |
| 34 | 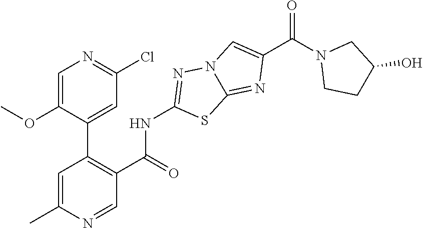 [SMILES: COc1cnc(Cl)cc1-c1cc(C)ncc1C(=O)Nc1nn2cc(C(=O)N3CC[C@@H](O)C3)nc2s1] | (R)-2′-chloro-N-(6-(3- hydroxypyrrolidine-1- carbonyl)imidazo[2,1- b][1,3,4]thiadiazol-2-y1)- 5′-methoxy-6-methyl- [4,4′-bipyridine]-3- carboxamide | LCMS (m/z): 514 [M + H]⁺. ¹H NMR (400 MHz, DMSO-d₆) δ 13.35 (s, 1H), 8.91 (s, 1H), 8.28 (s, 1H), 8.16 (s, 1H), 7.49 (s, 1H), 7.36 (s, 1H), 4.92 (s, 1H), 4.32-4.28 (m, 1H), 4.20- 3.86 (m, 2H), 3.66 (s, 3H), 3.59-3.47 (m, 2H), 2.58 (s, 3H), 2.03-1.71 (m, 2H) |
| 35 | 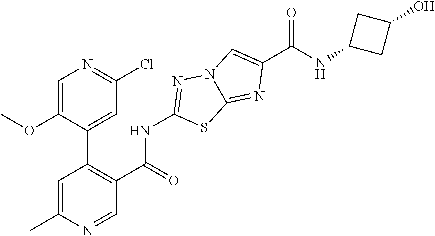 [SMILES: COc1cnc(Cl)cc1-c1cc(C)ncc1C(=O)Nc1nn2cc(C(=O)N[C@H]3C[C@@H](O)C3)nc2s1] | 2-(2′-chloro-5′-methoxy- 6-methyl-[4,4′- bipyridine]-3- carboxamido)-N-((1s,3s)- 3-hydroxycyclobutyl) imidazo[2,1-b]\|1,3,4] thiadiazole- 6-carboxamide | LCMS (m/z): 514 [M + H]⁺. ¹H NMR (300 MHz, DMSO-d₆) δ 13.40 (s, 1H), 8.87 (s, 1H), 8.38 (s, 1H), 8.24-8.17 (m, 2H), 7.57 (s, 1H), 7.45 (s, 1H), 6.54 (s, 1H), 5.03 (d, J = 6.0 Hz, 1H), 3.95-3.75 (m, 2H), 3.67 (s, 3H), 2.60 (s, 3H), 2.59- 2.51 (m, 1 H), 2.50-2.49 (s, 1H), 2.01-1.84 (m, 2H) |
| 36 | 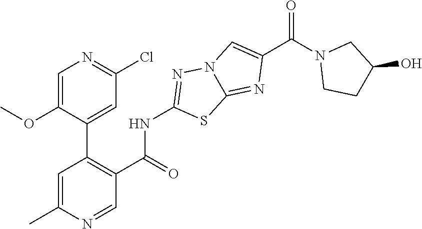 [SMILES: COc1cnc(Cl)cc1-c1cc(C)ncc1C(=O)Nc1nn2cc(C(=O)N3CC[C@H](O)C3)nc2s1] | (S)-2′-chloro-N-(6-(3- hydroxypyrrolidine-1- carbonyl)imidazo[2,1- b][1,3,4]thiadiazol-2-y1)- 5′-methoxy-6-methyl- [4,4′-bipyridine]-3- carboxamide | LCMS (m/z): 514 [M + H]⁺. ¹H NMR (300 MHz, DMSO-d₆) δ 13.36 (s, 1H), 8.85 (s, 1H), 8.45 (s, 1H), 8.20 (s, 1H), 7.60 (s, 1H), 7.49 (s, 1H), 4.32- 4.28 (m, 1H), 3.93 (s, 2H), 3.67 (s, 3H), 2.79-2.69 (m, 1H), 2.61 (s, 3H), 2.32-2.23 (m, 1H), 1.89 (s, 2H) |
| 37 | 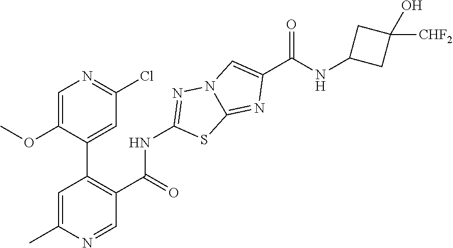 [SMILES: COc1cnc(Cl)cc1-c1cc(C)ncc1C(=O)Nc1nn2cc(C(=O)NC3CC(O)(C(F)F)C3)nc2s1] | 2-(2′-chloro-5′-methoxy- 6-methyl-[4,4′- bipyridine]-3- carboxamido)-N-(3- (difluoromethyl)-3- hydroxycyclobutyl) imidazo[2,1-b] [1,3,4]thiadiazole- 6-carboxamide | LCMS (m/z): 564 [M + H]⁺. ¹H NMR (400 MHz, DMSO-d₆) δ 13.40 (s, 1H), 8.88 (s, 1H), 8.38 (s, 1H), 8.19 (s, 1H), 7.54- 7.43 (m, 2H), 6.05-5.77 (m, 1H), 4.10-4.03 (m, 1H), 3.67 (s, 3H), 2.67-2.59 (m, 5H), 2.23-2.18 (m, 2H) |

### Example 38: Synthesis of 4-(2-Methoxyphenyl)-6-methyl-N-(thiazolo[4,5-c]pyridin-2-yl)nicotinamide

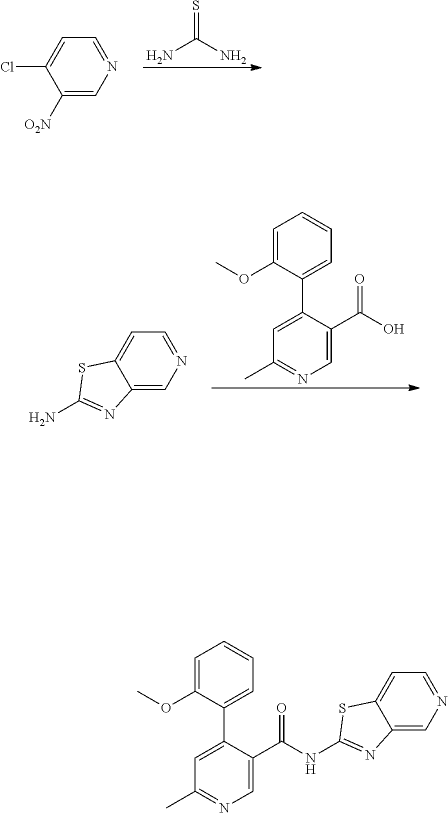

Step 1: To a solution of 4-chloro-3-nitropyridine (400 mg, 2.52 mmol) in sulfolane (4 mL) at room temperature was added thiourea (768 mg, 10.09 mmol) and the mixture was heated at 135° C. for 1.25 h under microwave irradiation. After cooling to room temperature, the mixture was diluted with water (40 mL), extracted with EtOAc (3×40 mL) and the combined organic layers washed with water (40 mL), brine (40 mL), dried over Na₂SO₄, filtered and concentrated under reduced pressure. The residue was purified by prep-TLC (DCM/MeOH=20:1, v/v) to give thiazolo[4,5-c]pyridin-2-amine (101 mg, 26% o) as a yellow solid. LCMS (ESI, m/z): 152 [M+H]⁺.

Step 2: To a solution of 4-(2-methoxyphenyl)-6-methylnicotinic acid (162 mg, 0.66 mmol) in DMF (5 mL) at room temperature was added HATU (381 mg, 1.0 mmol) and DIPEA (259 mg, 2.0 mmol) and the mixture was stirred for 30 min. Thiazolo[4,5-c]pyridin-2-amine (101 mg, 0.66 mmol) was added and the mixture was stirred at room temperature overnight. The mixture was diluted with water (50 mL), extracted with EtOAc (3×20 mL) and the combined organic layers were washed with water (50 mL), brine (50 mL), dried over Na₂SO₄, filtered and concentrated under reduced pressure. The residue was purified by prep-HPLC to give 4-(2-methoxyphenyl)-6-methyl-N-(thiazolo[4,5-c]pyridin-2-yl)nicotinamide (14 mg, 5%) as a white solid. LCMS (ESI, m/z): 377 [M+H]⁺, ¹H NMR (400 MHz, DMSO-d₆): δ 8.93 (s, 1H), 8.76 (s, 1H), 8.32 (s, 1H), 7.95 (s, 1H), 7.40-7.32 (m, 2H), 7.28 (s, 1H), 7.06 (t, J=7.4 Hz, 1H), 6.96 (d, J=8.2 Hz, 1H), 3.48 (s, 3H), 2.57 (s, 3H).

### Example 39: Synthesis of N-(6-Chlorothiazolo[4,5-c]pyridin-2-yl)-4-(2-methoxyphenyl)-6-methylnicotinamide

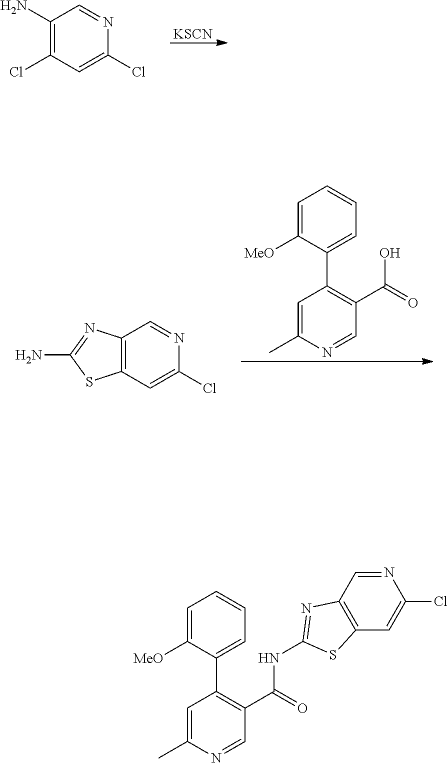

Step 1: To a solution of 4,6-dichloropyridin-3-amine (3.00 g, 18.4 mmol) and KSCN (5.37 g, 55.2 mmol) in 1,4-dioxane (60 mL) was added concentrated aqueous HCl (0.2 mL) and the mixture was heated at reflux for 48 h. The mixture was concentrated under reduced pressure, the residue was diluted with a mixture of DCM/MeOH (20/1, 20 mL) and water (20 mL) was added. The resulting precipitate was collected by filtration and dried under reduced pressure to give 6-chlorothiazolo[4,5-c]pyridin-2-amine (750 mg, 20%) as a grey solid. LCMS (ESI, m/z): 186 [M+H]⁺, ¹H NMR (400 MHz, DMSO-d₆): δ 8.32 (s, 1H), 7.93 (s, 2H), 7.88 (s, 1H).

Step 2: To a solution of 6-chlorothiazolo[4,5-c]pyridin-2-amine (400 mg, 2.15 mmol), 4-(2-methoxyphenyl)-6-methylnicotinic acid (629 mg, 2.59 mmol) and DIPEA (836 mg, 6.46 mmol) in DMF (10 mL) was added HATU (1.23 g, 3.23 mmol) and the mixture was heated at 80° C. overnight. The mixture was diluted with water (50 mL), extracted with EtOAc (2×50 mL) and the combined organic layers were washed with water (50 mL), brine (50 mL), dried over Na₂SO₄, filtered and concentrated under reduced pressure. The residue was purified by prep-TLC (DCM/MeOH=15/1, v/v) to give N-(6-chlorothiazolo[4,5-c]pyridin-2-yl)-4-(2-methoxyphenyl)-6-methylnicotinamide (320 mg, 36%) as a yellow solid. LCMS (ESI, m/z): 411 [M+H]⁺, ¹H NMR (400 MHz, DMSO-d₆): δ 8.84 (s, 1H), 8.72 (s, 1H), 8.21 (s, 1H), 7.44-7.36 (m, 2H), 7.34 (s, 1H), 7.09 (t, J=7.4 Hz, 1H), 6.97 (d, J=8.4 Hz, 1H), 3.47 (s, 3H), 2.59 (s, 3H).

### Example 40: Synthesis of N-(6-(4-Chlorophenyl)thiazolo[4,5-c]pyridin-2-yl)-4-(2-methoxyphenyl)-6-methylnicotinamide

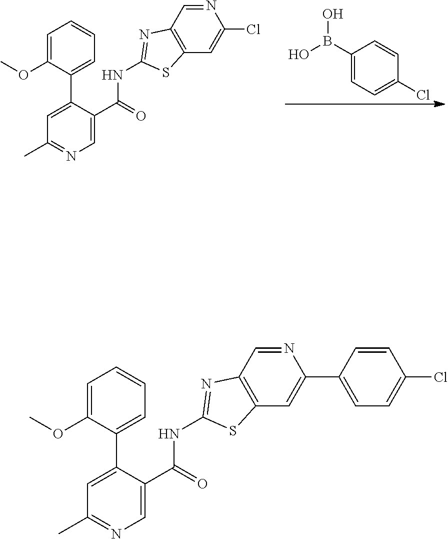 [SMILES: COc1ccccc1-c1cc(C)ncc1C(=O)Nc1nc2cnc(Cl)cc2s1 ; OB(O)c1ccc(Cl)cc1 ; COc1ccccc1-c1cc(C)ncc1C(=O)Nc1nc2cnc(-c3ccc(Cl)cc3)cc2s1]

To a mixture of N-(6-chlorothiazolo[4,5-c]pyridin-2-yl)-4-(2-methoxyphenyl)-6-methylnicotinamide (30 mg, 0.073 mmol), (4-chlorophenyl)boronic acid (12 mg, 0.073 mmol) and Na₂CO₃ (24 mg, 0.219 mmol) in 1,4-dioxane (3 mL) and water (0.6 mL) was added Pd(PPh₃)₄ (9 mg, 0.007 mmol) and the mixture was heated at reflux overnight. The mixture was diluted with water (30 mL), extracted with EtOAc (30 mL×2) and the combined organic layers washed with water (30 mL), brine (30 mL), dried over Na₂SO₄, filtered and concentrated under reduced pressure. The residue was purified by prep-TLC (DCM/MeOH=20/1, v/v) to afford N-(6-(4-chlorophenyl)thiazolo[4,5-c]pyridin-2-yl)-4-(2-methoxyphenyl)-6-methylnicotinamide (7 mg, 20%) as an off-white solid. LCMS (ESI, m/z): 487 [M+H]⁺, ¹H NMR (400 MHz, DMSO-d₆): δ 9.09 (s, 1H), 8.74 (s, 1H), 8.67 (s, 1H), 8.15 (d, J=8.4 Hz, 2H), 7.56 (d, J=8.2 Hz, 2H), 7.40 (t, J=6.8 Hz, 2H), 7.35 (s, 1H), 7.10 (t, J=7.4 Hz, 1H), 6.98 (d, J=8.4 Hz, 1H), 3.49 (s, 3H), 2.59 (s, 3H).

### Example 41: Synthesis of 4-(2-Methoxyphenyl)-6-methyl-N-(6-(piperidin-1-yl)thiazolo[4,5-c]pyridin-2-yl)nicotinamide

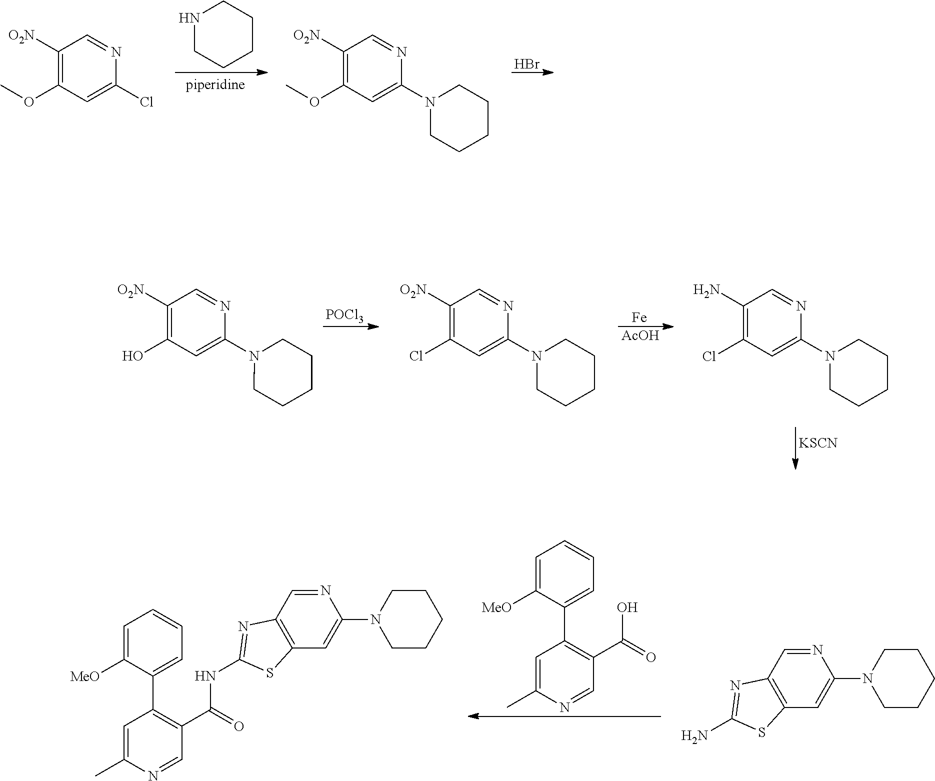

Step 1: To a solution of 2-chloro-4-methoxy-5-nitropyridine (200 mg, 1.06 mmol) and piperidine (181 mg, 2.12 mmol) in DMF (4 mL) was added K₂CO₃ (293 mg, 2.12 mmol) and the mixture was heated at 60° C. for 5 h in a sealed tube. The mixture was diluted with water (50 mL), extracted with EtOAc (2×50 mL) and the combined organic layers washed with water (50 mL), brine (50 mL), dried over Na₂SO₄, filtered and concentrated under reduced pressure to give 4-methoxy-5-nitro-2-(piperidin-1-yl)pyridine (260 mg, 99%) as a yellow solid. LCMS (ESI, m/z): 238 [M+H]⁺, ¹H NMR (400 MHz, DMSO-d₆): δ 8.73 (s, 1H), 6.33 (s, 1H), 3.95 (s, 3H), 3.73 (t, J=5.4 Hz, 4H), 1.71-1.60 (m, 2H), 1.59-1.50 (m, 4H).

Step 2: To a solution of 4-methoxy-5-nitro-2-(piperidin-1-yl)pyridine (260 mg, 1.1 mmol) in AcOH (1 mL) was added HBr (33% w/w in AcOH, 1 mL) and the mixture was heated at 105° C. in a sealed tube for 48 h. The mixture was concentrated under reduced pressure, diluted with water (50 mL), adjusted to pH 9-10 via addition of a saturated aqueous NaHCO₃ solution and extracted with EtOAc (2×50 mL). The combined organic layers were washed with water (50 mL), brine (50 mL), dried over Na₂SO₄, filtered and concentrated under reduced pressure.

The residue was purified by prep-TLC (Pet. Ether/EtOAc=10/1, v/v) to give 5-nitro-2-(piperidin-1-yl)pyridin-4-ol (90 mg, 37%) as a yellow solid. LCMS (ESI, m/z): 224 [M+H], ¹H NMR (400 MHz, DMSO-d₆): δ 11.2 (s, 1H), 8.78 (s, 1H), 6.22 (s, 1H), 3.65 (t, J=5.4 Hz, 4H), 1.71-1.59 (m, 2H), 1.59-1.48 (m, 4H).

Step 3: To a solution of 5-nitro-2-(piperidin-1-yl)pyridin-4-ol (90 mg, 0.04 mmol) in POCl₃ (1 mL) was added N,N-dimethylaniline (95 mg, 0.08 mmol) and the resulting mixture was heated at 100° C. in a sealed tube for 3 h. The mixture was concentrated under reduced pressure and the residue diluted with a saturated aqueous NaHCO₃ solution (50 mL) and extracted with EtOAc (2×50 mL). The combined organic layers were washed with water (50 mL), brine (50 mL), dried over Na₂SO₄, filtered and concentrated under reduced pressure to give 4-chloro-5-nitro-2-(piperidin-1-yl)pyridine (150 mg, 90%) as a brown solid. LCMS (ESI, m/z): 242 [M+H]⁺, ¹H NMR (400 MHz, DMSO-d₆): δ 8.89 (s, 1H), 7.09 (s, 1H), 3.79-3.70 (m, 4H), 1.71-1.61 (m, 2H), 1.60-1.51 (m, 4H).

Step 4: To a solution of 4-chloro-5-nitro-2-(piperidin-1-yl)pyridine (247 mg, 1.02 mmol) in AcOH (6 mL) at 75° C. was added Fe powder (228 mg, 4.09 mmol) and the mixture was heated at 75° C. for 3 h. The mixture was concentrated under reduced pressure and the residue was diluted with a saturated aqueous NaHCO₃ solution (50 mL) and filtered. The filtrate was extracted with EtOAc (2×40 mL) and the combined organic layers were washed with water (50 mL), brine (50 mL), dried over Na₂SO₄, filtered and concentrated under reduced pressure to give 4-chloro-6-(piperidin-1-yl)pyridin-3-amine (210 mg, 97%) as a yellow solid. LCMS (ESI, m/z): 212 [M+H]⁺, ¹H NMR (400 MHz, DMSO-d₆): δ 7.76 (s, 1H), 6.74 (s, 1H), 4.72 (s, 2H), 3.26 (t, J=4.5 Hz, 4H), 1.52 (s, 6H).

Step 5: To a solution of 4-chloro-6-(piperidin-1-yl)pyridin-3-amine (210 mg, 0.992 mmol) and KSCN (482 mg, 4.96 mmol) in 1,4-dioxane (5 mL) was added concentrated aqueous HCl (catalytic) and the mixture was heated at 110° C. in a sealed tube overnight. The mixture was concentrated under reduced pressure and the residue diluted with a saturated aqueous NaHCO₃ solution (40 mL) and extracted with EtOAc (2×50 mL). The combined organic layers were washed with water (40 mL), brine (40 mL), dried over Na₂SO₄, filtered and concentrated under reduced pressure. The residue was purified by prep-TLC (DCM/MeOH=20/1, v/v) to give 6-(piperidin-1-yl)thiazolo[4,5-c]pyridin-2-amine (100 mg, 43%) as a grey solid. LCMS (ESI, m/z): 235 [M+H]⁺, ¹H NMR (400 MHz, DMSO-d₆): δ 8.13 (s, 1H), 7.31 (s, 2H), 7.17 (s, 1H), 3.43-3.37 (m, 4H), 1.55 (s, 6H).

Step 6: To a mixture of 6-(piperidin-1-yl)thiazolo[4,5-c]pyridin-2-amine (50 mg, 0.213 mmol), 4-(2-methoxyphenyl)-6-methylnicotinic acid (62 mg, 0.26 mmol) and DIPEA (83 mg, 0.64 mmol) in DMF (4 mL) was added HATU (122 g, 0.32 mmol) and the mixture was heated at 80° C. overnight. The mixture was diluted with water (50 mL), extracted with EtOAc (2×50 mL) and the combined organic layers washed with water (50 mL), brine (50 mL), dried over Na₂SO₄, filtered and concentrated under reduced pressure. The residue was purified by prep-HPLC to give 4-(2-methoxyphenyl)-6-methyl-N-(6-(piperidin-1-yl)thiazolo[4,5-c]pyridin-2-yl)nicotinamide (21 mg, 20%) as an off-white solid. LCMS (ESI, m/z): 460 [M+H]⁺, ¹H NMR (400 MHz, DMSO-d₆): δ 12.6 (s, 1H), 8.68 (s, 1H), 8.56 (s, 1H), 7.43-7.27 (m, 4H), 7.08 (t, J=7.4 Hz, 1H), 6.97 (d, J=8.2 Hz, 1H), 3.54-3.47 (m, 7H), 2.58 (s, 3H), 1.58 (s, 6H).

### Example 42: Synthesis of N-(6-Bromothiazolo[4,5-b]pyridin-2-yl)-4-(2-methoxyphenyl)-6-methylnicotinamide

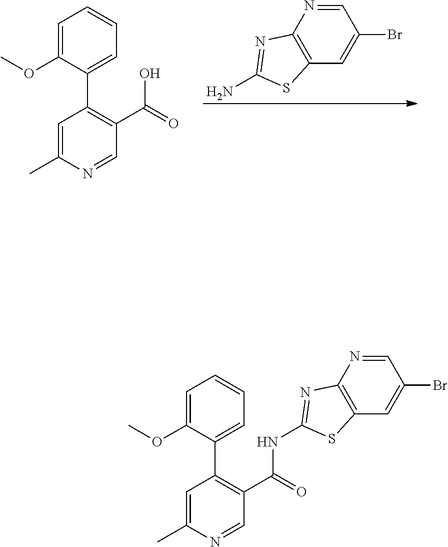 [SMILES: COc1ccccc1-c1cc(C)ncc1C(=O)O ; Nc1nc2ncc(Br)cc2s1 ; COc1ccccc1-c1cc(C)ncc1C(=O)Nc1nc2ncc(Br)cc2s1]

To a mixture of 6-bromothiazolo[4,5-b]pyridin-2-amine (100 mg, 0.435 mmol), 4-(2-methoxyphenyl)-6-methylnicotinic acid (127 mg, 0.522 mmol) and DIPEA (170 mg, 1.3 mmol) in DMF (2 mL) was added HATU (248 mg, 0.652 mmol) and the mixture was heated at 80° C. overnight. The mixture was diluted with water (50 mL), extracted with EtOAc (2×50 mL) and the combined organic layers washed with water (50 mL), brine (50 mL), dried over Na₂SO₄, filtered and concentrated under reduced pressure. The residue was purified by prep-TLC (DCM/MeOH=20/1, v/v) to give N-(6-bromothiazolo[4,5-b]pyridin-2-yl)-4-(2-methoxyphenyl)-6-methylnicotinamide (60 mg, 30%) as a yellow solid. LCMS (ESI, m/z): 455, 457 [M+H]⁺, ¹H NMR (400 MHz, DMSO-d₆): δ 8.74 (s, 1H), 8.67 (s, 1H), 8.60 (d, J=2.2 Hz, 1H), 7.38 (t, J=7.2 Hz, 2H), 7.31 (s, 1H), 7.08 (t, J=7.4 Hz, 1H), 6.96 (d, J=8.4 Hz, 1H), 3.48 (s, 3H), 2.58 (s, 3H).

### Example 43: Synthesis of N-(6-(4-Chlorophenyl)thiazolo[4,5-b]pyridin-2-yl)-4-(2-methoxyphenyl)-6-methylnicotinamide

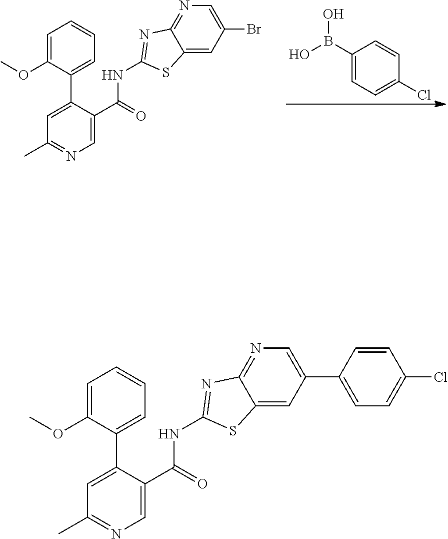 [SMILES: COc1ccccc1-c1cc(C)ncc1C(=O)Nc1nc2ncc(Br)cc2s1 ; OB(O)c1ccc(Cl)cc1 ; COc1ccccc1-c1cc(C)ncc1C(=O)Nc1nc2ncc(-c3ccc(Cl)cc3)cc2s1]

To a mixture of N-(6-bromothiazolo[4,5-b]pyridin-2-yl)-4-(2-methoxyphenyl)-6-methylnicotinamide (40 mg, 0.088 mmol), (4-chlorophenyl)boronic acid (21 mg, 0.13 mmol) and Na₂CO₃ (28 mg, 0.26 mmol) in 1,4-dioxane (4 mL) and water (0.8 mL) was added Pd(PPh₃)₄ (10 mg, 0.009 mmol) and the mixture was heated at reflux overnight. The mixture was diluted with water (50 mL), extracted with EtOAc (50 mL×2) and the combined organic layers washed with water (50 mL), brine (50 mL), dried over Na₂SO₄, filtered and concentrated under reduced pressure. The residue was purified by prep-HPLC to afford N-(6-(4-chlorophenyl)thiazolo[4,5-b]pyridin-2-yl)-4-(2-methoxyphenyl)-6-methylnicotinamide (20 mg, 48%) as an off-white solid. LCMS (ESI, m/z): 487 [M+H]⁺, ¹H NMR (400 MHz, DMSO-d₆): δ 8.86 (d, J=2.2 Hz, 1H), 8.78-8.68 (m, 2H), 7.80 (d, J=8.2 Hz, 2H), 7.57 (d, J=8.2 Hz, 2H), 7.39 (t, J=7.4 Hz, 2H), 7.33 (s, 1H), 7.10 (t, J=7.4 Hz, 1H), 6.97 (d, J=8.2 Hz, 1H), 3.50 (s, 3H), 2.59 (s, 3H).

TABLE M
The following examples were prepared using a similar procedure to that described
for Example 42 and 43

| Ex. | | | |
|---|---|---|---|
| No. | Structure | Compound Name | Characterization Data |
| 44 | 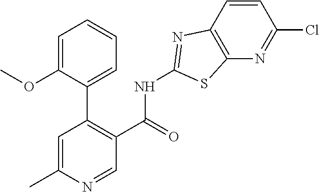 [SMILES: COc1ccccc1-c1cc(C)ncc1C(=O)Nc1nc2ccc(Cl)nc2s1] | N-(5- Chlorothiazolo[5,4- b]pyridin-2-y1)-4- (2-methoxyphenyl)- 6-methylnicotinamide | LCMS (m/z): 411 [M + H]⁺. ¹H NMR (400 MHz, DMSO-d₆) δ 13.0 (s, 1H), 8.72 (s, 1H), 8.17 (d, J = 8.4 Hz, 1H), 7.57 (d, J = 8.4 Hz, 1H), 7.45-7.36 (m, 2H), 7.33 (s, 1H), 7.13-7.06 (m, 1H), 6.97 (d, J = 8.4 Hz, 1H), 3.49 (s, 3H), 2.58 (s, 3H) |
| 45 | 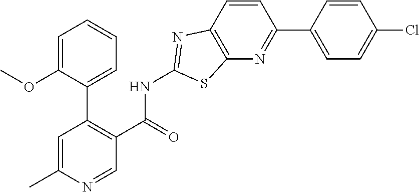 [SMILES: COc1ccccc1-c1cc(C)ncc1C(=O)Nc1nc2ccc(-c3ccc(Cl)cc3)nc2s1] | N-(5-(4- Chlorophenyl) thiazolo[5,4-b] pyridin-2- y1)-4-(2- methoxyphenyl)-6- methylnicotinamide | LCMS (m/z): 487 [M + H]⁺. ¹H NMR (400 MHz, DMSO-d₆) δ 12.9 (s, 1H), 8.73 (s, 1H), 8.22-8.14 (m, 3H), 8.12- 8.08 (m, 1H), 7.56 (d, J = 8.4 Hz, 2H), 7.43-7.37 (m, 2H), 7.34 (s, 1H), 7.15-7.06 (m, 1H), 7.00-6.94 (m, 1H), 3.51 (s, 3H), 2.59 (s, 3H) |
| 46 | 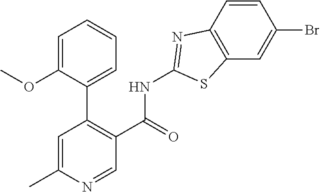 [SMILES: COc1ccccc1-c1cc(C)ncc1C(=O)Nc1nc2ccc(Br)cc2s1] | N-(6- Bromobenzo[d] thiazol-2-y1)-4-(2- methoxyphenyl)-6- methylnicotinamide | LCMS (m/z): 454, 456 [M + H]⁺. ¹H NMR (400 MHz, DMSO-d₆) δ 12.8 (s, 1H), 8.71 (s, 1H), 8.23 (s, 1H), 7.68 (d, J = 8.6 Hz, 1H), 7.57 (d, J = 8.6 Hz, 1H), 7.44-7.27 (m, 3H), 7.08 (t, J = 7.6 Hz, 1H), 6.96 (d, J = 8.4 Hz, 1H), 3.48 (s, 3H), 2.58 (s, 3H) |
| 47 | 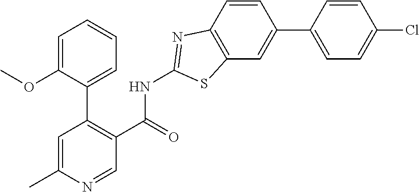 [SMILES: COc1ccccc1-c1cc(C)ncc1C(=O)Nc1nc2ccc(-c3ccc(Cl)cc3)cc2s1] | N-(6-(4- Chlorophenyl)benzo [d]thiazol-2-yl)-4- (2-methoxyphenyl)- 6-methylnicotinamide | LCMS (m/z): 486 [M + H]⁺. ¹H NMR (400 MHz, DMSO-d₆) δ 12.8 (s, 1H), 8.72 (s, 1H), 8.30 (d, J = 1.8 Hz, 1H), 7.82 (d, J = 8.4 Hz, 1H), 7.78-7.72 (m, 3H), 7.56-7.51 (m, 2H), 7.42- 7.36 (m, 2H), 7.33 (s, 1H), 7.09 (t, J = 7.4 Hz, 1H), 6.97 (d, J = 8.6 Hz, 1H), 3.49 (s, 3H), 2.59 (s, 3H) |
| 48 | 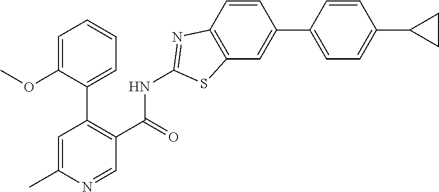 [SMILES: COc1ccccc1-c1cc(C)ncc1C(=O)Nc1nc2ccc(-c3ccc(C4CC4)cc3)cc2s1] | N-(6-(4- Cyclopropylphenyl) benzo[d]thiazol-2- y1)-4-(2- methoxyphenyl)-6- methylnicotinamide | LCMS (m/z): 492 [M + H]⁺. ¹H NMR (400 MHz, DMSO-d₆) δ 12.8 (s, 1H), 8.72 (s, 1H), 8.23 (d, J = 1.8 Hz, 1H), 7.79 (d, J = 8.6 Hz, 1H), 7.70 (dd, J = 8.6, 1.8 Hz, 1H), 7.60 (d, J = 8.0 Hz, 2H), 7.44-7.36 (m, 2H), 7.33 (s, 1H), 7.17 (d, J = 8.0 Hz, 2H), 7.09 (t, J = 7.6 Hz, 1H), 6.97 (d, J = 8.6 Hz, 1H), 3.50 (s, 3H), 2.59 (s, 3H), 1.96 (tt, J = 8.6, 5.2 Hz, 1H), 0.98 (dd, J = 8.2, 2.2 Hz, 2H), 0.71 (dd, J = 5.0, 1.8 Hz, 2H) |
| 49 | 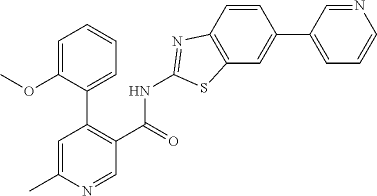 [SMILES: COc1ccccc1-c1cc(C)ncc1C(=O)Nc1nc2ccc(-c3cccnc3)cc2s1] | 4-(2- Methoxyphenyl)-6- methyl-N-(6- (pyridin-3- yl)benzo[d]thiazol- 2-yl)nicotinamide | LCMS (m/z): 453 [M + H]⁺. ¹H NMR (400 MHz, DMSO-d₆) δ 12.8 (s, 1H), 9.00-8.93 (m, 1H), 8.73 (s, 1H), 8.60- 8.55 (m, 1H), 8.40-8.34 (m, 1H), 8.17-8.11 (m, 1H), 7.88-7.78 (m, 2H), 7.54-7.48 (m, 1H), 7.42-7.36 (m, 2H), 7.34 (s, 1H), 7.12-7.06 (m, 1H), 7.00-6.95 (m, 1H), 3.49 (s, 3H), 2.59 (s, 3H) |
| 50 | 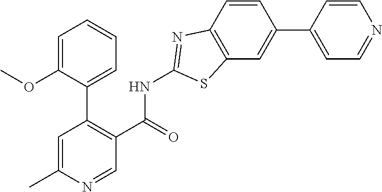 [SMILES: COc1ccccc1-c1cc(C)ncc1C(=O)Nc1nc2ccc(-c3ccncc3)cc2s1] | 4-(2- Methoxyphenyl)-6- methyl-N-(6- (pyridin-4- yl)benzo[d]thiazol- 2-yl)nicotinamide | LCMS (m/z): 453 [M + H]⁺. ¹H NMR (400 MHz, DMSO-d₆) δ 12.9 (s, 1H), 8.73 (s, 1H), 8.64 (d, J = 5.6 Hz, 2H), 8.49-8.46 (m, 1H), 7.91-7.84 (m, 2H), 7.79-7.75 (m, 2H), 7.42-7.37 (m, 2H), 7.33 (s, 1H), 7.13-7.06 (m, 1H), 7.00-6.95 (m, 1H), 3.49 (s, 3H), 2.59 (s, 3H) |
| 51 | 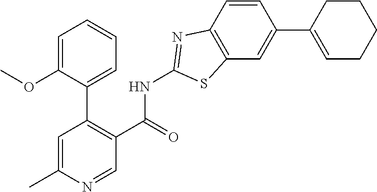 [SMILES: COc1ccccc1-c1cc(C)ncc1C(=O)Nc1nc2ccc(C3=CCCCC3)cc2s1] | N-(6-(Cyclohex-1- en-1- yl)benzo[d]thiazol- 2-y1)-4-(2- methoxyphenyl)-6- methylnicotinamide | LCMS (m/z): 456 [M + H]⁺. ¹H NMR (400 MHz, DMSO-d₆) δ 12.7 (s, 1H), 8.70 (s, 1H), 7.96 (d, J = 1.8 Hz, 1H), 7.66 (d, J = 8.4 Hz, 1H), 7.50 (dd, J = 8.4, 1.8 Hz, 1H), 7.42-7.36 (m, 2H), 7.32 (s, 1H), 7.08 (t, J = 7.4 Hz, 1H), 6.97 (d, J = 8.4 Hz, 1H), 6.20 (t, J = 1.8 Hz, 1H), 3.48 (s, 3H), 2.58 (s, 3H), 2.45-2.39 (m, 2H), 2.20 (q, J = 2.8 Hz, 2H), 1.79-1.71 (m, 2H), 1.66- 1.58 (m, 2H) |
| 52 | 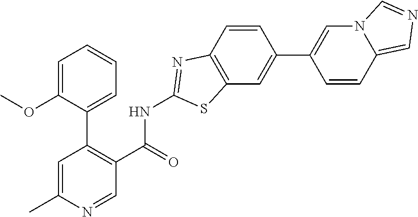 [SMILES: COc1ccccc1-c1cc(C)ncc1C(=O)Nc1nc2ccc(-c3ccc4cncn4c3)cc2s1] | N-(6-(Imidazo[1,5- a]pyridin-6- yl)benzo[d]thiazol- 2-y1)-4-(2- methoxyphenyl)-6- methylnicotinamide | LCMS (m/z): 492 [M + H]⁺. ¹H NMR (400 MHz, DMSO-d₆) δ 12.8 (s, 1H), 8.74 (d, J = 12.0 Hz, 2H), 8.42 (s, 1H), 8.31 (d, J = 1.8 Hz, 1H), 7.85 (d, J = 8.4 Hz, 1H), 7.77 (dd, J = 8.6, 1.8 Hz, 1H), 7.67 (d, J = 9.4 Hz, 1H), 7.45- 7.36 (m, 3H), 7.33 (s, 1H), 7.22 (d, J = 9.6 Hz, 1H), 7.09 (t, J = 7.4 Hz, 1H), 6.98 (d, J = 8.4 Hz, 1H), 3.50 (s, 3H), 2.59 (s, 3H) |
| 53 | 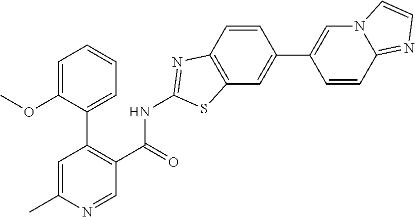 [SMILES: COc1ccccc1-c1cc(C)ncc1C(=O)Nc1nc2ccc(-c3ccc4nccn4c3)cc2s1] | N-(6-(Imidazo[1,2- a]pyridin-6- yl)benzo[d]thiazol- 2-y1)-4-(2- methoxyphenyl)-6- methylnicotinamide | LCMS (m/z): 492 [M + H]⁺. ¹H NMR (400 MHz, DMSO-d₆) δ 12.8 (s, 1H), 8.97 (s, 1H), 8.73 (s, 1H), 8.33 (d, J = 1.8 Hz, 1H), 7.97 (s, 1H), 7.86 (d, J = 8.4 Hz, 1H), 7.78 (dd, J = 8.4, 1.9 Hz, 1H), 7.69-7.64 (m, 2H), 7.61 (s, 1H), 7.42-7.36 (m, 2H), 7.33 (s, 1H), 7.09 (t, J = 7.4 Hz, 1H), 6.98 (d, J = 8.6 Hz, 1H), 3.50 (s, 3H), 2.59 (s, 3H) |
| 54 | 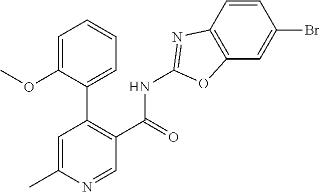 [SMILES: COc1ccccc1-c1cc(C)ncc1C(=O)Nc1nc2ccc(Br)cc2o1] | N-(6- Bromobenzo[d] oxazol-2-y1)-4-(2- methoxyphenyl)-6- methylnicotinamide | LCMS (m/z): 438, 440 [M + H]⁺. ¹H NMR (400 MHz, DMSO-d₆) δ 12.1 (s, 1H), 8.70 (s, 1H), 7.95 (s, 1H), 7.58- 7.48 (m, 2H), 7.39 (s, 1H), 7.33 (d, J = 6.6 Hz, 2H), 7.08-6.97 (m, 2H), 3.61 (s, 3H), 2.59 (s, 3H) |
| 55 | 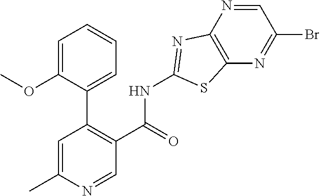 [SMILES: COc1ccccc1-c1cc(C)ncc1C(=O)Nc1nc2ncc(Br)nc2s1] | N-(6- Bromothiazolo[4,5- b]pyrazin-2-yl)-4- (2-methoxyphenyl)- 6-methylnicotinamide | LCMS (m/z): 456, 458 [M + H]⁺. ¹H NMR (400 MHz, DMSO-d₆) δ 13.4 (s, 1H), 8.77 (d, J = 9.4 Hz, 2H), 7.44- 7.39 (m, 2H), 7.37 (s, 1H), 7.14-7.07 (m, 1H), 6.98 (d, J = 8.4 Hz, 1H), 3.50 (s, 3H), 2.60 (s, 3H) |
| 56 | 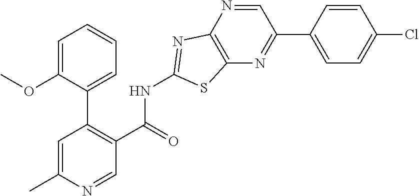 [SMILES: COc1ccccc1-c1cc(C)ncc1C(=O)Nc1nc2ncc(-c3ccc(Cl)cc3)nc2s1] | N-(6-(4- Chlorophenyl) thiazolo[4,5-b] pyrazin- 2-y1)-4-(2- methoxyphenyl)-6- methylnicotinamide | LCMS (m/z): 488 [M + H]⁺. ¹H NMR (400 MHz, DMSO-d₆) δ 13.3 (s, 1H), 9.26 (s, 1H), 8.76 (s, 1H), 8.23-8.18 (m, 2H), 7.63-7.59 (m, 2H), 7.44- 7.39 (m, 2H), 7.36 (s, 1H), 7.15-7.08 (m, 1H), 6.99 (d, J = 8.2 Hz, 1H), 3.52 (s, 3H), 2.60 (s, 3H) |
| 57 | 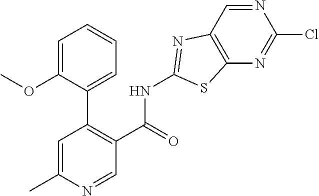 [SMILES: COc1ccccc1-c1cc(C)ncc1C(=O)Nc1nc2cnc(Cl)nc2s1] | N-(5- Chlorothiazolo[5,4- d]pyrimidin-2-y1)- 4-(2- methoxyphenyl)-6- methylnicotinamide | LCMS (m/z): 412 [M + H]⁺. |
| 58 | 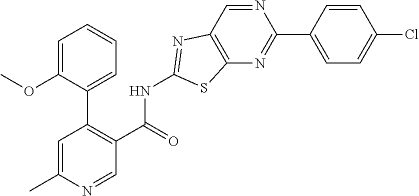 [SMILES: COc1ccccc1-c1cc(C)ncc1C(=O)Nc1nc2cnc(-c3ccc(Cl)cc3)nc2s1] | N-(5-(4- Chlorophenyl) thiazolo[5,4- d]pyrimidin-2-yl)- 4-(2- methoxyphenyl)-6- methylnicotinamide | LCMS (m/z): 488 [M + H]⁺. ¹H NMR (400 MHz, DMSO-d₆) δ 13.2 (s, 1H), 9.23 (s, 1H), 8.76 (s, 1H), 8.43 (d, J = 8.2 Hz, 2H), 7.59 (d, J = 8.2 Hz, 2H), 7.39 (d, J = 7.2 Hz, 2H), 7.34 (s, 1H), 7.10 (t, J = 7.4 Hz, 1H), 6.98 (d, J = 8.4 Hz, 1H), 3.51 (s, 3H), 2.59 (s, 3H) |
| 59 | 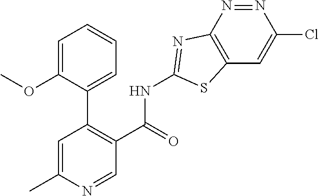 [SMILES: COc1ccccc1-c1cc(C)ncc1C(=O)Nc1nc2nnc(Cl)cc2s1] | N-(3- Chlorothiazolo[4,5- c]pyridazin-6-y1)-4- (2-methoxyphenyl)- 6-methylnicotinamide | LCMS (m/z): 412 [M + H]⁺. ¹H NMR (400 MHz, DMSO-d₆) δ 13.5 (s, 1H), 8.75 (s, 1H), 8.54 (s, 1H), 7.41 (d, J = 7.6 Hz, 2H), 7.35 (s, 1H), 7.10 (t, J = 7.6 Hz, 1H), 6.97 (d, J = 8.2 Hz, 1H), 3.49 (s, 3H), 2.59 (s, 3H) |
| 60 | 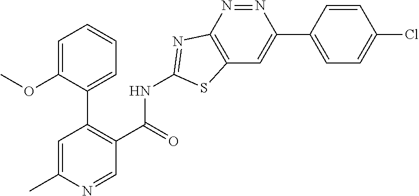 [SMILES: COc1ccccc1-c1cc(C)ncc1C(=O)Nc1nc2nnc(-c3ccc(Cl)cc3)cc2s1] | N-(3-(4- Chlorophenyl) thiazolo[4,5-c] pyridazin- 6-y1)-4-(2- methoxyphenyl)-6- methylnicotinamide | LCMS (m/z): 488 [M + H]⁺. ¹H NMR (400 MHz, DMSO-d₆) δ 8.84 (s, 1H), 8.68 (s, 1H), 8.17 (t, J = 7.6 Hz, 2H), 7.60 (d, J = 8.2 Hz, 2H), 7.36 (q, J = 8.4 Hz, 2H), 7.25 (s, 1H), 7.06 (t, J = 7.4 Hz, 1H), 6.97 (d, J = 8.2 Hz, 1H), 3.53 (s, 3H), 2.58 (s, 3H) |
| 61 | 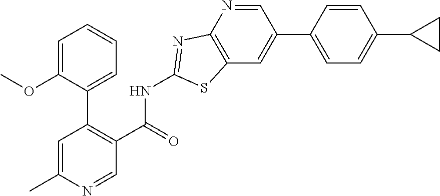 [SMILES: COc1ccccc1-c1cc(C)ncc1C(=O)Nc1nc2ncc(-c3ccc(C4CC4)cc3)cc2s1] | N-(6-(4- Cyclopropylphenyl) thiazolo[4,5- b]pyridin-2-y1)-4- (2-methoxyphenyl)- 6-methylnicotinamide | LCMS (m/z): 493 [M + H]⁺. 1H NMR (400 MHz, DMSO-d₆) δ 13.1 (s, 1H), 8.83 (d, J = 2.4 Hz, 1H), 8.75 (s, 1H), 8.69 (d, J = 2.4 Hz, 1H), 7.64 (d, J = 8.0 Hz, 2H), 7.45-7.32 (m, 3H), 7.21 (d, J = 8.0 Hz, 2H), 7.11 (d, J = 7.4 Hz, 1H), 6.98 (d, J = 8.2 Hz, 1H), 3.50 (s, 3H), 2.59 (s, 3H), 2.03-1.94 (m, 1H), 0.99 (dd, J = 8.2, 2.2 Hz, 2H), 0.73 (dd, J = 4.8, 1.8 Hz, 2H) |
| 62 | 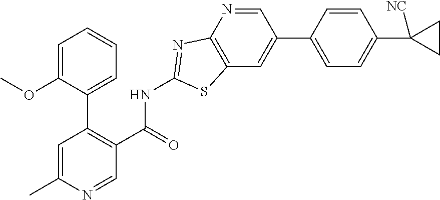 [SMILES: COc1ccccc1-c1cc(C)ncc1C(=O)Nc1nc2ncc(-c3ccc(C4(C#N)CC4)cc3)cc2s1] | N-(6-(4-(1- Cyanocyclopropyl) phenyl)thiazolo[4,5- b]pyridin-2-yl)-4- (2-methoxyphenyl)- 6-methylnicotinamide | LCMS (m/z): 518 [M + H]⁺. ¹H NMR (400 MHz, DMSO-d₆) δ 13.1 (s, 1H), 8.87 (d, J = 2.2 Hz, 1H), 8.74 (d, J = 2.6 Hz, 2H), 7.80 (d, J = 8.0 Hz, 2H), 7.47 (d, J = 8.0 Hz, 2H), 7.40 (t, J = 7.6 Hz, 2H), 7.34 (s, 1H), 7.10 (t, J = 7.4 Hz, 1H), 6.97 (d, J = 8.2 Hz, 1H), 3.50 (s, 3H), 2.59 (s, 3H), 1.80 (q, J = 5.0 Hz, 2H), 1.58 (q, J = 5.0 Hz, 2H) |
| 63 | 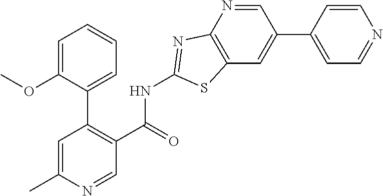 [SMILES: COc1ccccc1-c1cc(C)ncc1C(=O)Nc1nc2ncc(-c3ccncc3)cc2s1] | 4-(2- Methoxyphenyl)-6- methyl-N-(6- (pyridin-4- yl)thiazolo[4,5- b]pyridin-2- yl)nicotinamide | LCMS (m/z): 454 [M + H]⁺. ¹H NMR (400 MHz, DMSO-d₆) δ 13.2 (s, 1H), 9.02 (d, J = 2.2 Hz, 1H), 8.91 (d, J = 2.2 Hz, 1H), 8.75 (s, 1H), 8.70 (d, J = 5.2 Hz, 2H), 7.85 (d, J = 5.2 Hz, 2H), 7.40 (t, J = 8.0 Hz, 2H), 7.35 (s, 1H), 7.11 (t, J = 7.4 Hz, 1H), 6.98 (d, J = 8.2 Hz, 1H), 3.50 (s, 3H), 2.60 (s, 3H) |
| 64 | 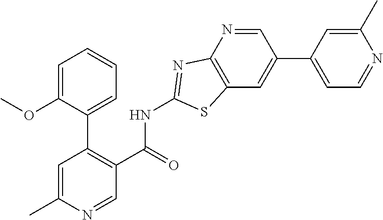 | 4-(2- Methoxyphenyl)-6- methyl-N-(6-(2- methylpyridin-4- yl)thiazolo[4,5- b]pyridin-2- yl)nicotinamide | LCMS (m/z): 468 [M + H]⁺. ¹H NMR (400 MHz, DMSO-d₆) δ 13.2 (s, 1H), 8.98 (d, J = 2.4 Hz, 1H), 8.87 (d, J = 2.4 Hz, 1H), 8.75 (s, 1H), 8.54 (d, J = 5.4 Hz, 1H), 7.71 (s, 1H), 7.62 (d, J = 5.4 Hz, 1H), 7.40 (t, J = 7.8 Hz, 2H), 7.34 (s, 1H), 7.10 (t, J = 7.6 Hz, 1H), 6.98 (d, J = 8.2 Hz, 1H), 3.50 (s, 3H), 2.59 (s, 3H), 2.56 (s, 3H) |
| 65 | 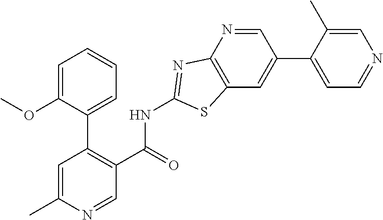 [SMILES: COc1ccccc1-c1cc(C)ncc1C(=O)Nc1nc2ncc(-c3ccncc3C)cc2s1] | 4-(2- Methoxyphenyl)-6- methyl-N-(6-(3- methylpyridin-4- yl)thiazolo[4,5- b]pyridin-2- yl)nicotinamide | LCMS (m/z): 468 [M + H]⁺. ¹H NMR (400 MHz, DMSO-d₆) δ 13.1 (s, 1H), 8.75 (s, 1H), 8.61 (d, J = 2.2 Hz, 1H), 8.57 (d, J = 1.8 Hz, 2H), 8.51 (d, J = 5.0 Hz, 1H), 7.43-7.33 (m, 4H), 7.10 (t, J = 7.4 Hz, 1H), 6.98 (d, J = 8.2 Hz, 1H), 3.50 (s, 3H), 2.59 (s, 3H), 2.32 (s, 3H) |
| 66 | 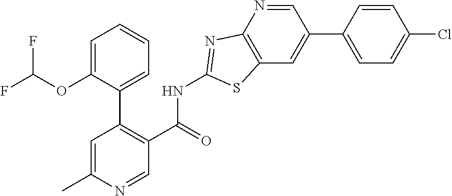 [SMILES: Cc1cc(-c2ccccc2OC(F)F)c(C(=O)Nc2nc3ncc(-c4ccc(Cl)cc4)cc3s2)cn1] | N-(6-(4- Chlorophenyl) thiazolo[4,5-b] pyridin-2- y1)-4-(2- (difluoromethoxy) phenyl)-6- methylnicotinamide | LCMS (m/z): 523 [M + H]⁺. ¹H NMR (400 MHz, DMSO-d₆) δ 13.3 (s, 1H), 8.90-8.87 (m, 2H), 8.74 (d, J = 2.2 Hz, 1H), 7.83-7.78 (m, 2H), 7.60- 7.56 (m, 2H), 7.52-7.45 (m, 2H), 7.37 (d, J = 5.0 Hz, 2H), 7.19 (d, J = 8.0 Hz, 1H), 7.04 (t, J = 73.6 Hz, 1H), 2.61 (s, 3H) |
| 67 | 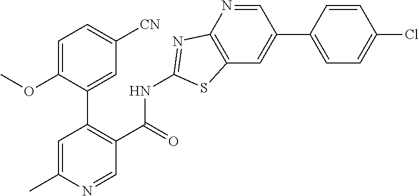 [SMILES: COc1ccc(C#N)cc1-c1cc(C)ncc1C(=O)Nc1nc2ncc(-c3ccc(Cl)cc3)cc2s1] | N-(6-(4- Chlorophenyl) thiazolo[4,5-b] pyridin-2- y1)-4-(5-cyano-2- methoxyphenyl)-6- methylnicotinamide | LCMS (m/z): 512 [M + H]⁺. ¹H NMR (400 MHz, DMSO-d₆) δ 13.2 (s, 1H), 8.91-8.78 (m, 2H), 8.74 (d, J = 2.4 Hz, 1H), 7.95-7.87 (m, 2H), 7.80 (d, J = 8.4 Hz, 2H), 7.58 (d, J = 8.4 Hz, 2H), 7.44 (s, 1H), 7.17 (d, J = 9.2 Hz, 1H), 3.58 (s, 3H), 2.60 (s, 3H) |
| 68 | 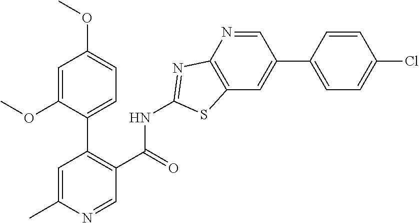 [SMILES: COc1ccc(-c2cc(C)ncc2C(=O)Nc2nc3ncc(-c4ccc(Cl)cc4)cc3s2)c(OC)c1] | N-(6-(4- Chlorophenyl) thiazolo[4,5-b] pyridin-2- yl)-4-(2,4- dimethoxyphenyl)- 6-methylnicotinamide | LCMS (m/z): 517 [M + H]⁺. ¹H NMR (400 MHz, DMSO-d₆) δ 13.1 (s, 1H), 8.88 (d, J = 2.2 Hz, 1H), 8.76 (d, J = 2.2Hz, 1H), 8.70 (s, 1H), 7.86-7.76 (m, 2H), 7.61-7.54 (m, 2H), 7.35 (d, J = 8.6 Hz, 1H), 7.30 (s, 1H), 6.69 (dd, J = 8.6, 2.2 Hz, 1H), 6.52 (d, J = 2.2 Hz, 1H), 3.80 (s, 3H), 3.49 (s, 3H), 2.58 (s, 3H) |
| 69 | 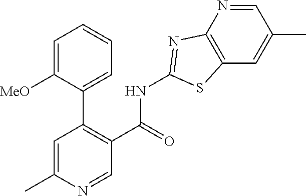 [SMILES: COc1ccccc1-c1cc(C)ncc1C(=O)Nc1nc2ncc(C)cc2s1] | 4-(2- Methoxyphenyl)-6- methyl-N-(6- methylthiazolo[4,5- b]pyridin-2- yl)nicotinamide | LCMS (m/z): 391 [M + H]⁺. ¹H NMR (400 MHz, DMSO-d₆) δ 12.9 (s, 1H), 8.72 (s, 1H), 8.40 (s, 1H), 8.23 (s, 1H), 7.42-7.37 (m, 2H), 7.33 (s, 1H), 7.09 (t, J = 7.4 Hz, 1H), 6.97 (d, J = 8.4Hz, 1H), 3.48 (s, 3H), 2.59 (s, 3H), 2.40 (s, 3H |
| 70 | 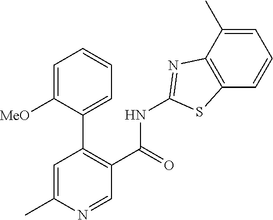 [SMILES: COc1ccccc1-c1cc(C)ncc1C(=O)Nc1nc2c(C)cccc2s1] | 4-(2- Methoxyphenyl)-6- methyl-N-(4- methylbenzo[d] thiazol-2- yl)nicotinamide | LCMS (m/z): 390 [M + H]⁺. ¹H NMR (400 MHz, DMSO-d₆) δ 12.8 (s, 1H), 8.71 (s, 1H), 7.76 (d, J = 7.8 Hz, 1H), 7.37 (d, J = 7.4 Hz, 2H), 7.32-7.15 (m, 3H), 7.08 (s, 1H), 6.98 (s, 1H), 3.49 (s, 3H), 2.59 (s, 3H), 2.58 (s, 3H) |
| 71 | 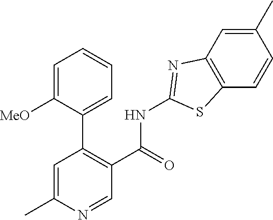 [SMILES: COc1ccccc1-c1cc(C)ncc1C(=O)Nc1nc2cc(C)ccc2s1] | 4-(2- Methoxyphenyl)-6- methyl-N-(5- methylbenzo[d] thiazol-2- yl)nicotinamide | LCMS (m/z): 390 [M + H]⁺. ¹H NMR (400 MHz, DMSO-d₆) δ 12.7 (s, 1H), 8.69 (s, 1H), 7.81 (d, J = 8.0 Hz, 1H), 7.56 (s, 1H), 7.37 (dd, J = 7.6, 2.0 Hz, 2H), 7.31 (s, 1H), 7.14 (dd, J = 8.0, 1.6 Hz, 1H), 7.08 (d, J = 7.4 Hz, 1H), 6.96 (d, J = 8.6 Hz, 1H), 3.47 (s, 3H), 2.57 (s, 3H), 2.42 (s, 3H) |
| 72 | 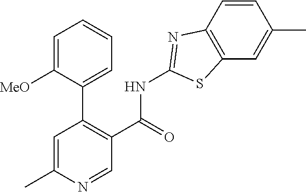 [SMILES: COc1ccccc1-c1cc(C)ncc1C(=O)Nc1nc2ccc(C)cc2s1] | 4-(2- Methoxyphenyl)-6- methyl-N-(6- methylbenzo[d] thiazol-2- yl)nicotinamide | LCMS (m/z): 390 [M + H]⁺. ¹H NMR (400 MHz, DMSO-d₆) δ 12.6 (s, 1H), 8.70 (s, 1H), 7.74 (s, 1H), 7.63 (d, J = 8.2 Hz, 1H), 7.38 (td, J = 7.4, 1.6 Hz, 2H), 7.32 (s, 1H), 7.25 (dd, J = 8.4, 1.8 Hz, 1H), 7.08 (t, J = 7.4 Hz, 1H), 6.96 (d, J = 8.4 Hz, 1H), 3.48 (s, 3H), 2.58 (s, 3H), 2.41 (s, 3H) |
| 73 | 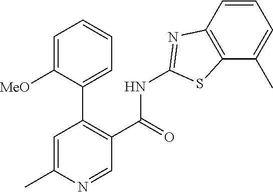 [SMILES: COc1ccccc1-c1cc(C)ncc1C(=O)Nc1nc2cccc(C)c2s1] | 4-(2- Methoxyphenyl)-6- methyl-N-(7- methylbenzo[d] thiazol-2- yl)nicotinamide | LCMS (m/z): 390 [M + H]⁺. ¹H NMR (400 MHz, DMSO-d₆) δ 12.7 (s, 1H), 8.71 (s, 1H), 7.59 (d, J = 8.0 Hz, 1H), 7.44-7.36 (m, 3H), 7.34 (s, 1H), 7.17- 7.06 (m, 2H), 6.97 (d, J = 8.2 Hz, 1H), 3.49 (s, 3H), 2.58 (s, 3H), 2.49 (s, 3H) |
| 74 | 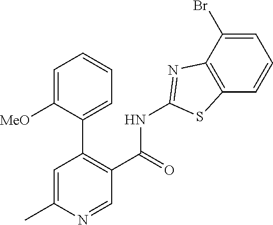 [SMILES: COc1ccccc1-c1cc(C)ncc1C(=O)Nc1nc2c(Br)cccc2s1] | N-(4- Bromobenzo[d] thiazol-2-y1)-4-(2- methoxyphenyl)-6- methylnicotinamide | LCMS (m/z): 454, 456 [M + H]⁺. ¹H NMR (400 MHz, DMSO-d₆) δ 13.2 (s, 1H), 8.73 (s, 1H), 7.99 (dd, J = 7.8, 1.2 Hz, 1H), 7.69 (dd, J = 7.8, 1.2 Hz, 1H), 7.39 (ddd, J = 7.2, 4.2, 2.6 Hz, 2H), 7.32 (s, 1H), 7.24 (t, J = 8.0 Hz, 1H), 7.09 (t, J = 7.4 Hz, 1H), 6.96 (d, J = 8.6 Hz, 1H), 3.48 (s, 3H), 2.58 (s, 3H) |
| 75 | 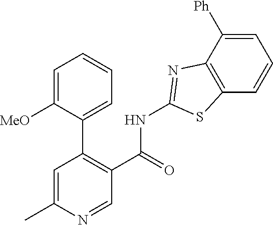 [SMILES: COc1ccccc1-c1cc(C)ncc1C(=O)Nc1nc2c(-c3ccccc3)cccc2s1] | 4-(2- Methoxyphenyl)-6- methyl-N-(4- phenylbenzo[d] thiazol-2- yl)nicotinamide | LCMS (m/z): 452 [M + H]⁺. ¹H NMR (400 MHz, DMSO-d₆) δ 12.8 (s, 1H), 8.70 (s, 1H), 8.01-7.92 (m, 1H), 7.82- 7.71 (m, 2H), 7.57-7.45 (m, 3H), 7.38 (tt, J = 8.6, 2.8 Hz, 4H), 7.30 (s, 1H), 7.08 (t, J = 7.4 Hz, 1H), 6.97 (d, J = 8.2 Hz, 1H), 3.48 (s, 3H), 2.57 (s, 3H) |
| 76 | 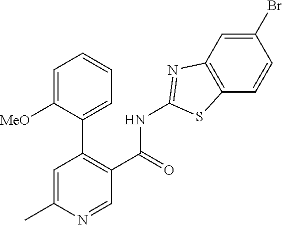 [SMILES: COc1ccccc1-c1cc(C)ncc1C(=O)Nc1nc2cc(Br)ccc2s1] | N-(5- Bromobenzo[d] thiazol-2-y1)-4-(2- methoxyphenyl)-6- methylnicotinamide | LCMS (m/z): 454, 456 [M + H]⁺. ¹H NMR (400 MHz, DMSO-d₆) δ 12.9 (s, 1H), 8.71 (s, 1H), 7.99-7.90 (m, 2H), 7.47 (dd, J = 8.6, 1.8 Hz, 1H), 7.41- 7.35 (m, 2H), 7.33 (s, 1H), 7.08 (t, J = 7.4 Hz, 1H), 6.96 (d, J = 8.6 Hz, 1H), 3.47 (s, 3H), 2.58 (s, 3H) |
| 77 | 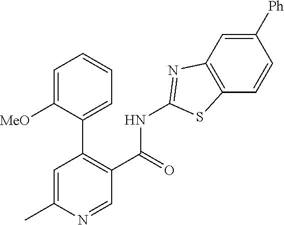 [SMILES: COc1ccccc1-c1cc(C)ncc1C(=O)Nc1nc2cc(-c3ccccc3)ccc2s1] | 4-(2- Methoxyphenyl)-6- methyl-N-(5- phenylbenzo[d] thiazol-2- yl)nicotinamide | LCMS (m/z): 452 [M + H]⁺. ¹H NMR (400 MHz, DMSO-d₆) δ 12.8 (s, 1H), 8.73 (s, 1H), 8.09-7.95 (m, 2H), 7.75 (d, J = 7.6 Hz, 2H), 7.61 (dd, J = 8.2, 1.8 Hz, 1H), 7.49 (t, J = 7.6 Hz, 2H), 7.42-7.30 (m, 4H), 7.09 (t, J = 7.6 Hz, 1H), 6.98 (d, J = 8.6 Hz, 1H), 3.50 (s, 3H), 2.59 (s, 3H) |
| 78 | 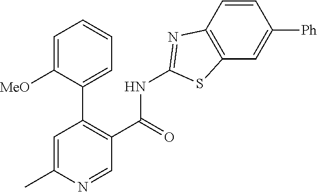 [SMILES: COc1ccccc1-c1cc(C)ncc1C(=O)Nc1nc2ccc(-c3ccccc3)cc2s1] | 4-(2- Methoxyphenyl)-6- methyl-N-(6- phenylbenzo[d] thiazol-2- yl)nicotinamide | LCMS (m/z): 452 [M + H]⁺. ¹H NMR (400 MHz, DMSO-d₆) δ 12.8 (s, 1H), 8.74 (s, 1H), 8.29 (d, J = 1.8 Hz, 1H), 7.85-7.71 (m, 4H), 7.49 (t, J = 7.6 Hz, 2H), 7.42-7.32 (m, 4H), 7.10 (t, J = 7.4 Hz, 1H), 6.98 (d, J = 8.6 Hz, 1H), 3.50 (s, 3H), 2.60 (s, 3H) |
| 79 | 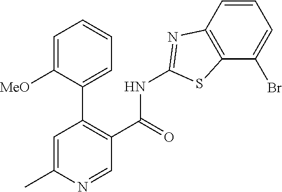 [SMILES: COc1ccccc1-c1cc(C)ncc1C(=O)Nc1nc2cccc(Br)c2s1] | N-(7- Bromobenzo[d] thiazol-2-y1)-4-(2- methoxyphenyl)-6- methylnicotinamide | LCMS (m/z): 454, 456 [M + H]⁺. ¹H NMR (400 MHz, DMSO-d₆) δ 12.9 (s, 1H), 8.7 (s, 1H), 7.75 (d, J = 8.0 Hz, 1H), 7.51 (d, J = 7.8 Hz, 1H), 7.44- 7.34 (m, 3H), 7.31 (s, 1H), 7.09 (t, J = 7.4 Hz, 1H), 6.98 (d, J = 8.2 Hz, 1H), 3.49 (s, 3H), 2.58 (s, 3H) |
| 80 | 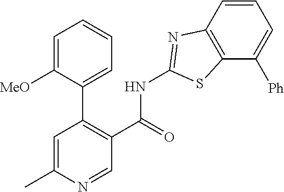 [SMILES: COc1ccccc1-c1cc(C)ncc1C(=O)Nc1nc2cccc(-c3ccccc3)c2s1] | 4-(2- Methoxyphenyl)-6- methyl-N-(7- phenylbenzo[d] thiazol-2- yl)nicotinamide | LCMS (m/z): 452 [M + H]⁺. ¹H NMR (400 MHz, DMSO-d₆) δ 12.8 (s, 1H), 8.71 (s, 1H), 7.78 (d, J = 8.0 Hz, 1H), 7.70 (d, J = 7.2 Hz, 2H), 7.60-7.50 (m, 3H), 7.46 (d, J = 7.6 Hz, 1H), 7.43-7.33 (m, 3H), 7.31 (s, 1H), 7.07 (t, J = 7.4 Hz, 1H), 6.95 (d, J = 8.6 Hz, 1H), 3.48 (s, 3H), 2.58 (s, 3H) |
| 81 | 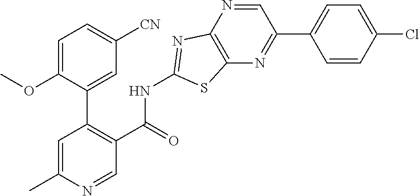 [SMILES: COc1ccc(C#N)cc1-c1cc(C)ncc1C(=O)Nc1nc2ncc(-c3ccc(Cl)cc3)nc2s1] | N-(6-(4- Chlorophenyl) thiazolo[4,5-b] pyrazin- 2-y1)-4-(5-cyano-2- methoxyphenyl)-6- methylnicotinamide | LCMS (m/z): 513 [M + H]⁺. ¹H NMR (400 MHz, DMSO-d₆) δ 13.4 (s, 1H), 9.26 (s, 1H), 8.83 (s, 1H), 8.21 (d, J = 8.6 Hz, 2H), 7.94-7.88 (m, 2H), 7.65- 7.58 (m, 2H), 7.46 (s, 1H), 7.18 (d, J = 9.2 Hz, 1H), 3.61 (s, 3H), 2.61 (s, 3H) |
| 82 | 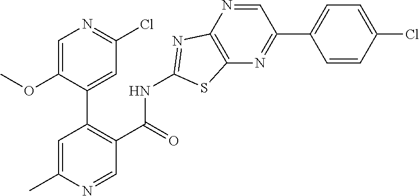 [SMILES: COc1cnc(Cl)cc1-c1cc(C)ncc1C(=O)Nc1nc2ncc(-c3ccc(Cl)cc3)nc2s1] | 2′-Chloro-N-(6-(4- chlorophenyl) thiazolo[4,5-b] pyrazin-2- yl)-5′-methoxy-6- methyl-[4,4′- bipyridine]-3- carboxamide | LCMS (m/z): 523 [M + H]⁺. ¹H NMR (400 MHz, DMSO-d₆) δ 13.5 (s, 1H), 9.23 (s, 1H), 8.91 (s, 1H), 8.26-8.15 (m, 3H), 7.60 (d, J = 9.0 Hz, 3H), 7.46 (s, 1H), 3.64 (s, 3H), 2.61 (s, 3H) |
| 83 | 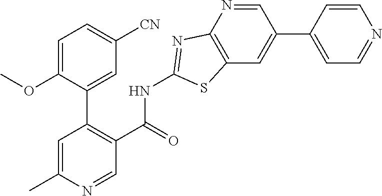 [SMILES: COc1ccc(C#N)cc1-c1cc(C)ncc1C(=O)Nc1nc2ncc(-c3ccncc3)cc2s1] | 4-(5-Cyano-2- methoxyphenyl)-6- methyl-N-(6- (pyridin-4- yl)thiazolo[4,5- b]pyridin-2- yl)nicotinamide | LCMS (m/z): 479 [M + H]⁺. ¹H NMR (400 MHz, DMSO-d₆) δ 13.2 (s, 1H), 9.01 (d, J = 2.2 Hz, 1H), 8.90 (d, J = 2.2 Hz, 1H), 8.82 (s, 1H), 8.69 (d, J = 6.2 Hz, 2H), 7.91 (d, J = 7.0 Hz, 2H), 7.85-7.80 (m, 2H), 7.44 (s, 1H), 7.17 (d, J = 9.2 Hz, 1H), 3.58 (s, 3H), 2.61 (s, 3H) |
| 84 | 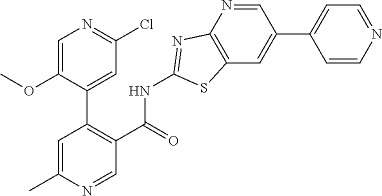 [SMILES: COc1cnc(Cl)cc1-c1cc(C)ncc1C(=O)Nc1nc2ncc(-c3ccncc3)cc2s1] | 2′-Chloro-5′- methoxy-6-methyl- N-(6-(pyridin-4- yl)thiazolo[4,5- b]pyridin-2-yl)- [4,4′-bipyridine]-3- carboxamide | LCMS (m/z): 489 [M + H]⁺. ¹H NMR (400 MHz, DMSO-d₆) δ 13.3 (s, 1H), 9.32-8.66 (m, 4H), 8.19 (s, 3H), 7.69- 7.40 (m, 3H), 3.65 (s, 3H), 2.63 (s, 3H) |
| 85 | 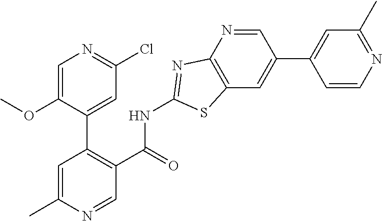 | 2′-Chloro-5′- methoxy-6-methyl- N-(6-(2- methylpyridin-4- yl)thiazolo[4,5- b]pyridin-2-yl)- [4,4′-bipyridine]-3- carboxamide | LCMS (m/z): 503 [M + H]⁺. ¹H NMR (400 MHz, DMSO-d₆) δ 13.4 (s, 1H), 9.00 (d, J = 2.2 Hz, 1H), 8.89 (d, J = 2.4 Hz, 2H), 8.55 (d, J = 5.2 Hz, 1H), 8.15 (d, J = 17.4 Hz, 1H), 7.72 (s, 1H), 7.65-7.60 (m, 2H), 7.48 (s, 1H), 3.61 (s, 3H), 2.62 (s, 3H), 2.56 (s, 3H) |

### Example 86: Synthesis of N-(6-Benzylbenzo[d]thiazol-2-yl)-4-(2-methoxyphenyl)-6-methylnicotinamide

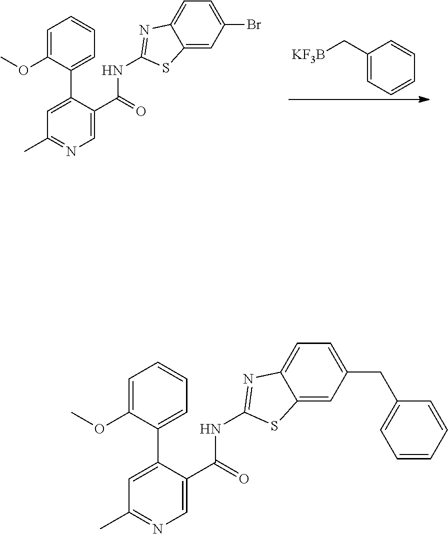

A mixture of N-(6-bromobenzo[d]thiazol-2-yl)-4-(2-methoxyphenyl)-6-methylnicotinamide (50 mg, 0.11 mmol), potassium benzyltrifluoroborate (32.7 mg, 0.17 mmol), Pd(OAc)₂ (2 mg, 8.8 μmol), RuPhos (8.7 mg, 0.19 mmol) and K₃PO4 (122 mg, 0.57 mmol) in toluene (2 mL) and water (0.4 mL) was heated at 130° C. under microwave irradiation for 2 h. The mixture was filtered and the filtrate concentrated under reduced pressure. The residue was purified by prep-HPLC to give N-(6-benzylbenzo[d]thiazol-2-yl)-4-(2-methoxyphenyl)-6-methylnicotinamide (4 mg, 4%) as a white solid. LCMS (ESI, m/z): 466 [M+H]⁺, ¹H NMR (400 MHz, DMSO-d₆): δ 12.7 (s, 1H), 8.70 (s, 1H), 7.82 (s, 1H), 7.65 (d, J=8.2 Hz, 1H), 7.42-7.23 (m, 8H), 7.18 (td, J=6.2, 2.8 Hz, 1H), 7.08 (t, J=7.6 Hz, 1H), 6.95 (d, J=8.6 Hz, 1H), 4.04 (s, 2H), 3.47 (s, 3H), 2.58 (s, 3H).

### Example 87: Synthesis of 4-(2-Methoxyphenyl)-6-methyl-N-(6-(pyridin-2-yl)benzo[d]thiazol-2-yl)nicotinamide

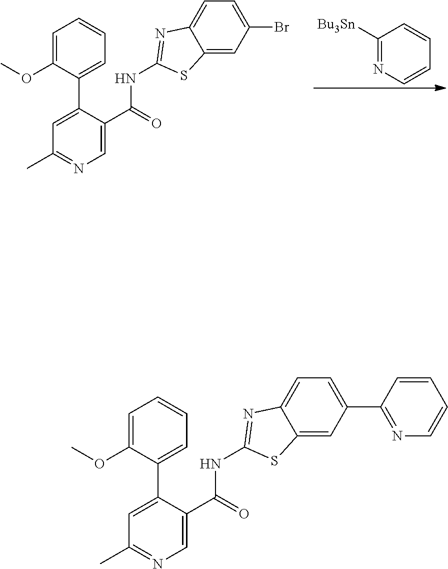

To a solution of N-(6-bromobenzo[d]thiazol-2-yl)-4-(2-methoxyphenyl)-6-methylnicotinamide (100 mg, 0.22 mmol) in toluene (1.5 mL) was added 2-(tributylstannyl)pyridine (89 mg, 0.24 mmol) and Pd(PPh₃)₄ (25 mg, 0.02 mmol) and the mixture was heated at reflux overnight. The mixture was diluted with water (20 mL), extracted with EtOAc (2×20 mL) and the combined organic phases were washed with water (20 mL), brine (20 mL), dried over Na₂SO₄, filtered and concentrated under reduced pressure. The residue was purified by prep-TLC (DCM/MeOH=25/1, v/v) to afford 4-(2-methoxyphenyl)-6-methyl-N-(6-(pyridin-2-yl)benzo[d]thiazol-2-yl)nicotinamide (20 mg, 20%) as a white solid. LCMS (ESI, m/z): 453 [M+H]⁺, ¹H NMR (400 MHz, DMSO-d₆): δ 12.8 (s, 1H), 8.71 (d, J=10.6 Hz, 2H), 8.67 (d, J=4.7 Hz, 1H), 8.20 (d, J=8.5 Hz, 1H), 8.03 (d, J=8.0 Hz, 1H), 7.89 (t, J=7.5 Hz, 1H), 7.84 (d, J=8.5 Hz, 1H), 7.44-7.31 (m, 4H), 7.10 (t, J=7.4 Hz, 1H), 6.98 (d, J=8.6 Hz, 1H), 3.50 (s, 3H), 2.59 (s, 3H).

### Example 88: Synthesis of (Racemic)-4-(2-Methoxyphenyl)-6-methyl-N-(6-((tetrahydrofuran-2-yl)methyl)benzo[d]thiazol-2-yl)nicotinamide

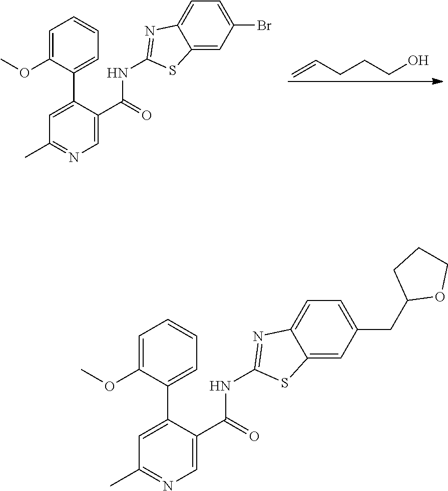

To a solution of N-(6-bromobenzo[d]thiazol-2-yl)-4-(2-methoxyphenyl)-6-methylnicotinamide (50 mg, 0.11 mmol) in DMF (2 mL) was added pent-4-en-1-ol (19 mg, 0.22 mmol), Pd₂(dba)₃ (5 mg, 5.5 μmol), DPE-phos (6 mg, 11 μmol) and t-BuONa (32 mg, 0.33 mmol) and the mixture was heated at 105° C. in a sealed tube overnight. The mixture was poured into water (20 mL), extracted with EtOAc (10 mL×4) and the combined organic phases were washed with brine (10 mL), dried over Na₂SO₄, filtered and concentrated under reduced pressure. The residue was purified by prep-TLC (DCM/MeOH=25/1, v/v) to give (Racemic)-4-(2-methoxyphenyl)-6-methyl-N-(6-((tetrahydrofuran-2-yl)methyl)benzo[d]thiazol-2-yl)nicotinamide (15 mg, 30%) as a yellow solid. LCMS (ESI, m/z): 460 [M+H]⁺, ¹H NMR (400 MHz, DMSO-d₆): δ 12.6 (s, 1H), 8.68 (s, 1H), 7.77 (s, 1H), 7.64 (d, J=8.4 Hz, 1H), 7.41-7.34 (m, 2H), 7.33-7.27 (m, 2H), 7.08 (t, J=7.4 Hz, 1H), 6.95 (d, J=8.4 Hz, 1H), 4.01-3.96 (m, 1H), 3.79-3.73 (m, 2H), 3.61 (s, 3H), 2.88-2.77 (m, 2H), 2.57 (s, 3H), 1.91-1.72 (m, 3H), 1.58-1.42 (m, 1H).

### Example 89: Synthesis of (Racemic)-4-(2-Methoxyphenyl)-6-methyl-N-(6-(tetrahydrofuran-2-yl)benzo[d]thiazol-2-yl)nicotinamide

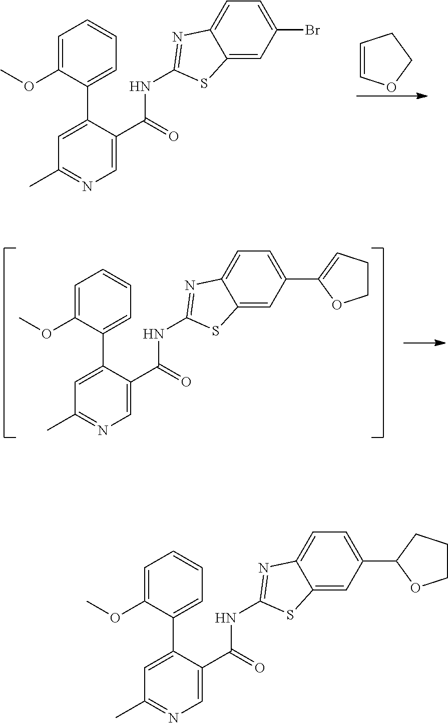

To a solution of N-(6-bromobenzo[d]thiazol-2-yl)-4-(2-methoxyphenyl)-6-methylnicotinamide (50 mg, 0.110 mmol) and 2,3-dihydrofuran (38.6 mg, 0.550 mmol) in DMF (2 mL) was added Pd(OAc)₂ (2.5 mg, 0.011 mmol), PPh₃ (5.77 mg, 0.022 mmol) and K₂CO₃ (30.4 mg, 0.220 mmol) and the mixture was heated at 110° C. in a sealed tube overnight. The mixture was allowed to cool to room temperature then placed under a hydrogen atmosphere (1 atm) and stirred at room temperature overnight. The mixture was diluted with water, extracted with EtOAc (10 mL×5) and the combined organic phases washed with brine (10 mL×2), dried over Na₂SO₄, filtered and concentrated under reduced pressure. The residue was purified by prep-TLC (DCM/MeOH=50/1, v/v) to afford (Racemic) 4-(2-methoxyphenyl)-6-methyl-N-(6-(tetrahydrofuran-2-yl)benzo[d]thiazol-2-yl)nicotinamide (15 mg, 31%). LCMS (ESI, m/z): 446 [M+H]⁺; ¹H NMR (400 MHz, DMSO-d₆): δ 12.7 (s, 1H), 8.71 (s, 1H), 7.89 (s, 1H), 7.69 (d, J=8.2 Hz, 1H), 7.42-7.35 (m, 3H), 7.33 (s, 1H), 7.08 (t, J=7.4 Hz, 1H), 6.96 (d, J=8.4 Hz, 4.90 (t, J=7.2 Hz, 1H), 4.02 (q, J=7.2 Hz, 1H), 3.82 (q, J=7.2 Hz, 1H), 3.48 (s),3H, 2.58 (s, 3H), 2.36-2.28t(m, 1H), 2.01-1.92 (m, 2H), 1.76-1.66 (m, 1H).

### Example 90: Synthesis of N-(6-Cyclohexylbenzo[d]thiazol-2-yl)-4-(2-methoxyphenyl)-6-methylnicotinamide

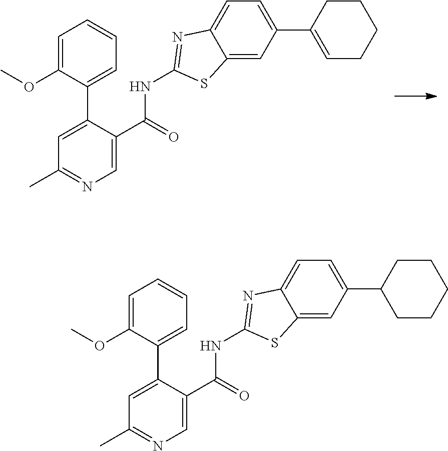 [SMILES: COc1ccccc1-c1cc(C)ncc1C(=O)Nc1nc2ccc(C3=CCCCC3)cc2s1 ; COc1ccccc1-c1cc(C)ncc1C(=O)Nc1nc2ccc(C3CCCCC3)cc2s1]

To a solution of N-(6-(cyclohex-1-en-1-yl)benzo[d]thiazol-2-yl)-4-(2-methoxyphenyl)-6-methylnicotinamide (45 mg, 0.098 mmol) in MeOH (5 mL) was added 20% Pd/C (10 mg) and the mixture was stirred under a H₂ atmosphere (1 atm) overnight. The mixture was filtered, the filtrate was concentrated, and the residue purified by prep-HPLC to afford N-(6-cyclohexylbenzo[d]thiazol-2-yl)-4-(2-methoxyphenyl)-6-methylnicotinamide (5 mg, 11%) as a white solid. L CMS (ESI, m/z): 458 [M+H]⁺, ¹H NMR (400 MHz, DMSO-d₆): δ 12.6 (s, 1H), 8.70 (s, 1H), 7.79 (s, 1H), 7.64 (d, J=8.2 Hz, 1H), 7.42-7.35 (m, 2H), 7.34-7.27 (m, 2H), 7.08 (t, J=7.4 Hz, 1H), 6.96 (d, J=8.4 Hz, 1H), 3.48 (s, 3H), 2.69-2.57 (m, 1H), 2.58 (s, 3H), 1.88-1.76 (m, 4H), 1.76-1.67 (m, 1H), 1.52-1.32 (m, 4H), 1.31-1.24 (m, 1H).

### Example 91: Synthesis of 4-(2-Methoxyphenyl)-6-methyl-N-(6-(piperidin-1-yl)benzo[d]thiazol-2-yl)nicotinamide

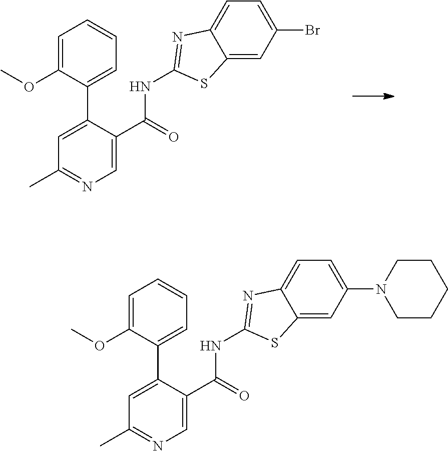 [SMILES: COc1ccccc1-c1cc(C)ncc1C(=O)Nc1nc2ccc(Br)cc2s1 ; COc1ccccc1-c1cc(C)ncc1C(=O)Nc1nc2ccc(N3CCCCC3)cc2s1]

A mixture of N-(6-bromobenzo[d]thiazol-2-yl)-4-(2-methoxyphenyl)-6-methylnicotinamide (30 mg, 0.066 mmol), piperidine (11 mg, 0.13 mmol), Pd₂(dba)₃·CHCl₃ (7 mg, 0.006 mmol), RuPhos (6 mg, 0.013 mmol) and Cs₂CO₃ (64 mg, 0.198 mmol) in dioxane (2 mL) was heated at 130° C. under microwave irradiation for 3 h. The mixture was filtered and the filtrate concentrated under reduced pressure. The residue was purified by prep-HPLC to afford 4-(2-methoxyphenyl)-6-methyl-N-(6-(piperidin-1-yl)benzo[d]thiazol-2-yl)nicotinamide (2 mg, 6%) as a yellow solid. LCMS (ESI, m/z): 459 [M+H]⁺, ¹H NMR (400 MHz, DMSO-d₆): δ 12.5 (s, 1H), 8.68 (s, 1H), 7.56 (d, J=8.8 Hz, 1H), 7.45-7.34 (m, 3H), 7.30 (s, 1H), 7.14-7.04 (m, 2H), 6.96 (d, J=8.2 Hz, 1H), 3.49 (s, 3H), 3.17-3.10 (m, 4H), 2.57 (s, 3H), 1.69-1.60 (m, 4H), 1.58-1.49 (m, 2H).

TABLE N
The following examples were prepared using a similar procedure to that described for Example 91

| Ex. | | | |
|---|---|---|---|
| No. | Structure | Compound Name | Characterization Data |
| 92 | 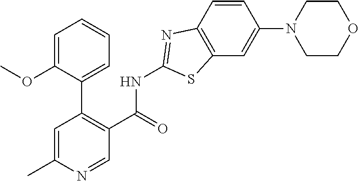 [SMILES: COc1ccccc1-c1cc(C)ncc1C(=O)Nc1nc2ccc(N3CCOCC3)cc2s1] | 4-(2-methoxyphenyl)- 6-methyl-N-(6-morph- olinobenzo[d]thiazol- 2-yl)nicotinamide | LCMS (m/z): 461 [M + H]⁺. ¹H NMR (400 MHz, DMSO- d₆) δ 12.5 (s, 1H), 8.69 (s, 1H), 7.60 (d, J = 8.8 Hz, 1H), 7.47-7.44 (m, 1H), 7.41-7.35 (m, 2H), 7.31 (s, 1H), 7.16- 7.11 (m, 1H), 7.11-7.05 (m, 1H), 6.97 (d, J = 8.2 Hz, 1H), 3.79-3.73 (m, 4H), 3.49 (s, 3H), 3.16-3.09 (m, 4H), 2.58 (s, 3H) |
| 93 | 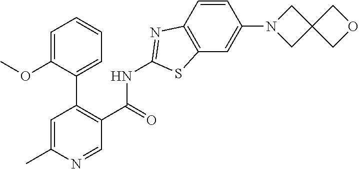 [SMILES: COc1ccccc1-c1cc(C)ncc1C(=O)Nc1nc2ccc(N3CC4(COC4)C3)cc2s1] | N-(6-(2-oxa-6-azaspiro [3.3]heptan-6-yl)benzo [d]thiazol-2-yl)-4-(2- methoxyphenyl)-6- methylnicotinamide | LCMS (m/z): 473 [M + H]⁺. ¹H NMR (400 MHz, DMSO- d₆) δ 12.5 (s, 1H), 8.67 (s, 1H), 7.55 (d, J = 8.6 Hz, 1H), 7.40-7.34 (m, 2H), 7.30 (s, 1H), 7.12-7.03 (m, 1H), 7.00- 6.93 (m, 2H), 6.62-6.53 (m, 1H), 4.72 (s, 4H), 4.00 (s, 4H), 3.48 (s, 3H), 2.57 (s, 3H) |
| 94 | 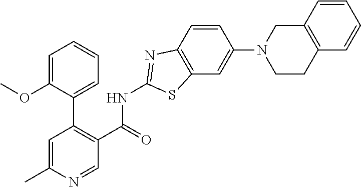 [SMILES: COc1ccccc1-c1cc(C)ncc1C(=O)Nc1nc2ccc(N3CCc4ccccc4C3)cc2s1] | N-(6-(3,4-dihydroiso- quinolin-2(1H)-yl)- benzo[d]thiazol-2-yl)- 4-(2-methoxyphenyl)- 6-methylnicotinamide | LCMS (m/z): 507 [M + H]⁺. ¹H NMR (400 MHz, DMSO- d₆) δ 12.5 (s, 1H), 8.68 (s, 1H), 7.61 (d, J = 8.8 Hz, 1H), 7.52 (d, J = 2.4 Hz, 1H), 7.38 (t, J = 7.6 Hz, 2H), 7.31 (s, 1H), 7.26-7.15 (m, 5H), 7.08 (t, J = 7.6 Hz, 1H), 6.97 (d, J = 8.2 Hz, 1H), 4.41 (s, 2H), 3.57 (t, J = 5.8 Hz, 2H), 3.49 (s, 3H), 2.94 (t, J = 5.6 Hz, 2H), 2.57 (s, 3H) |
| 95 | 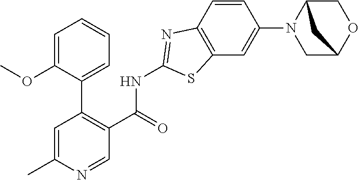 [SMILES: COc1ccccc1-c1cc(C)ncc1C(=O)Nc1nc2ccc(N3C[C@@H]4C[C@H]3CO4)cc2s1] | N-(6-((1S,4S)-2-oxa- 5-azabicyclo[2.2.1] heptan-5-yl)benzo[d] thiazol-2-yl)-4-(2- methoxyphenyl)-6- methylnicotinamide | LCMS (m/z): 473 [M + H]⁺. ¹H NMR (400 MHz, DMSO- d₆) δ 12.4 (s, 1H), 8.67 (s, 1H), 7.54 (d, J = 8.8 Hz, 1H), 7.37 (d, J = 7.6 Hz, 2H), 7.30 (s, 1H), 7.13 (d, J = 2.4 Hz, 1H), 7.07 (t, J = 7.4 Hz, 1H), 6.97 (d, J = 8.2 Hz, 1H), 6.78 (dd, J = 8.8, 2.4 Hz, 1H), 4.60 (d, J = 11.2 Hz, 2H), 3.76 (d, J = 7.4 Hz, 1H), 3.69 (d, J = |
| | | | 7.4 Hz, 1H), 3.54 (d, J = 9.2 |
| | | | Hz, 1H), 3.50 (s, 3H), 3.00 |
| | | | (d, J = 9.4 Hz, 1H), 2.57 (s, |
| | | | 3H), 1.94 (d, J = 8.8 Hz, 1H), |
| | | | 1.85 (d, J = 9.8 Hz, 1H) |
| 96 | 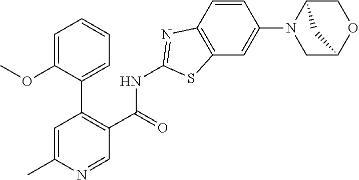 [SMILES: COc1ccccc1-c1cc(C)ncc1C(=O)Nc1nc2ccc(N3C[C@H]4C[C@@H]3CO4)cc2s1] | N-(6-((1R,4R)-2-oxa- 5-azabicyclo[2.2.1] heptan-5-yl)benzo[d] thiazol-2-yl)-4-(2- methoxyphenyl)-6- methylnicotinamide | LCMS (m/z): 473 [M + H]⁺. ¹H NMR (400 MHz, DMSO- d₆) δ 12.4 (s, 1H), 8.67 (s, 1H), 7.54 (d, J = 8.8 Hz, 1H), 7.37 (d, J = 8.0 Hz, 2H), 7.30 (s, 1H), 7.13 (d, J = 2.4 Hz, 1H), 7.07 (t, J = 7.6 Hz, 1H), 6.97 (d, J = 8.0 Hz, 1H), 6.78 (dd, J = 8.8, 2.4 Hz, 1H), 4.62 (d, J = 11.2 Hz, 2H), 3.76 (d, J = 7.6 Hz, 1H), 3.69 (d, J = |
| | | | 7.2 Hz, 1H), 3.54 (d, J = 9.2 |
| | | | Hz, 1H), 3.50 (s, 3H), 3.00 (d, |
| | | | J = 9.4 Hz, 1H), 2.57 (s, 3H), |
| | | | 1.94 (d, J = 8.8 Hz, 1H), 1.85 |
| | | | (d, J = 9.6 Hz, 1H) |

### Example 97: Synthesis of N-(6-(4-Chlorophenyl)thiazolo[4,5-c]pyridin-2-yl)-4-(5-cyano-2-methoxyphenyl)-6-methylnicotinamide

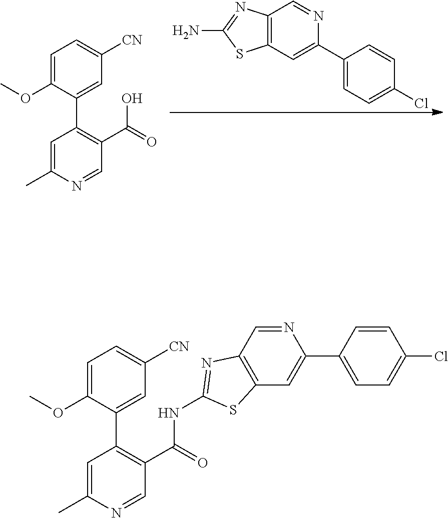 [SMILES: COc1ccc(C#N)cc1-c1cc(C)ncc1C(=O)O ; Nc1nc2cnc(-c3ccc(Cl)cc3)cc2s1 ; COc1ccc(C#N)cc1-c1cc(C)ncc1C(=O)Nc1nc2cnc(-c3ccc(Cl)cc3)cc2s1]

To a solution of 4-(5-cyano-2-methoxyphenyl)-6-methylnicotinic acid (50 mg, 0.18 mmol) in NMP (2 mL) was added HATU (106 mg, 0.28 mmol), DIPEA (72 mg, 0.56 mmol) and 6-(4-chlorophenyl)thiazolo[4,5-c]pyridin-2-amine (58 mg, 0.22 mmol) and the mixture was heated at 80° C. overnight. The mixture was diluted with water (50 mL), extracted with EtOAc (30 mL×3) and the combined organic layers washed with brine (30 mL×2), dried over Na₂SO₄, filtered and concentrated under reduced pressure. The residue was purified by prep-TLC (DCM/MeOH=30/1, v/v) to afford N-(6-(4-chlorophenyl)thiazolo[4,5-c]pyridin-2-yl)-4-(5-cyano-2-methoxyphenyl)-6-methylnicotinamide (15 mg, 16%), as a white solid. LCMS (ESI, m/z): 512 [M+H]⁺, ¹H NMR (400 MHz, DMSO-d₆): δ 13.1 (s, 1H), 9.10 (s, 1H), 8.81 (s, 1H), 8.67 (s, 1H), 8.15 (d, J=8.6 Hz, 2H), 7.93-7.89 (m, 2H), 7.61-7.51 (d, J=8.8 Hz, 2H), 7.44 (s, 1H), 7.17 (d, J=9.2 Hz, 1H), 3.57 (s, 3H), 2.60 (s, 3H).

### Example 98: Synthesis of N-(6-(4-Chlorophenyl)thiazolo[4,5-c]pyridin-2-yl)-4-(2-(difluoromethoxy)phenyl)-6-methylnicotinamide

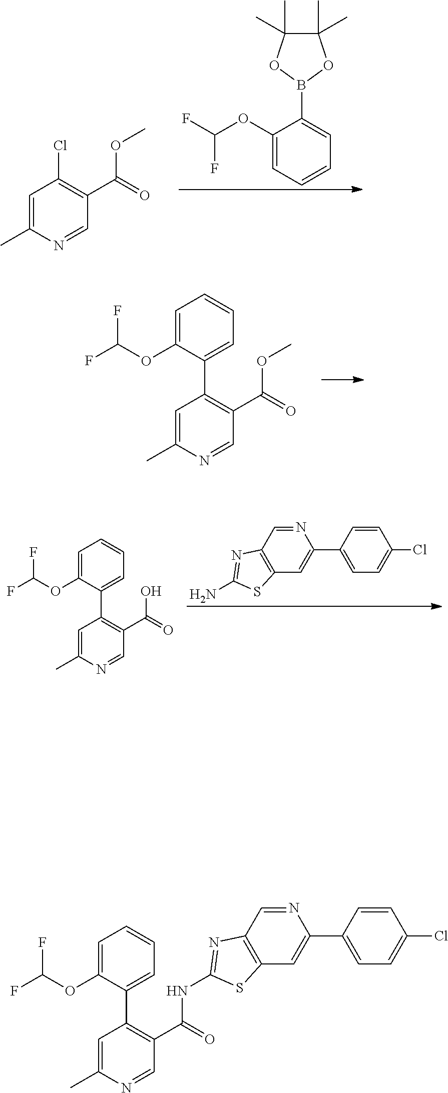

Step 1: To a solution of methyl 4-chloro-6-methylnicotinate (481 mg, 2.59 mmol) in 1,4-dioxane (25 mL) was added 2-(2-(difluoromethoxy)phenyl)-4,4,5,5-tetramethyl-1,3,2-dioxaborolane (700 mg, 2.59 mmol), Pd(PPh₃)₄ (149 mg, 0.12 mmol) and Cs₂CO₃ (2.53 g, 7.77 mmol) and the mixture was heated at 85° C. overnight. The mixture was diluted with water (100 mL), extracted with EtOAc (3×30 mL) and the combined organic layers washed with water (100 mL), brine (100 mL), dried over Na₂SO₄, filtered and concentrated under reduced pressure. The residue was purified by silica gel chromatography (6-10% EtOAc/Pet. ether) to give methyl 4-(2-(difluoromethoxy)phenyl)-6-methylnicotinate (440 mg, 58%) as a yellow oil. LCMS (ESI, m/z): 294 [M+H], ¹H NMR (400 MHz, DMSO-d₆): δ 8.89 (s, 1H), 7.50 (td, J=7.6, 2.0 Hz, 1H), 7.43-7.21 (m, 4H), 7.08 (t, J=73.6 Hz, 1H), 3.64 (s, 3H), 2.57 (s, 3H).

Step 2: To a solution of methyl 4-(2-(difluoromethoxy)phenyl)-6-methylnicotinate (200 mg, 0.63 mmol) in water (2 mL) and MeOH (2 mL) was added NaOH (81 mg, 2.04 mmol) and the mixture was heated at 50° C. for 2 h. The mixture was acidified to pH 3 with 1 M aqueous HCl and extracted with DCM/MeOH (50/1, 3×30 mL). The combined organic layers were dried over Na₂SO₄, filtered and concentrated under reduced pressure to give 4-(2-(difluoromethoxy)phenyl)-6-methylnicotinic acid (200 mg) as a yellow solid, which was used without further purification. LCMS (ESI, m/z): 280 [M+H]⁺.

Step 3: To a solution of 4-(2-(difluoromethoxy)phenyl)-6-methylnicotinic acid (50 mg, 0.17 mmol) in DMF (2 mL) was added HATU (102 mg, 0.26 mmol) and DIPEA (69 mg, 0.53 mmol) and the mixture was stirred at room temperature for 30 min. 6-(4-Chlorophenyl)thiazolo[4,5-c]pyridin-2-amine (56 mg, 0.21 mmol) was added and the mixture was heated at 85° C. overnight. The mixture was diluted with water (20 mL), extracted with EtOAc (3×20 mL) and the combined organic layers were washed with water (20 mL), brine (20 mL), dried over Na₂SO₄, filtered and concentrated under reduced pressure. The residue was purified by prep-HPLC to give N-(6-(4-chlorophenyl)thiazolo[4,5-c]pyridin-2-yl)-4-(2-(difluoromethoxy)phenyl)-6-methylnicotinamide (37 mg, 39%) as a white solid. LCMS (ESI, m/z): 523 [M+H]⁺, ¹H NMR (400 MHz, DMSO-d₆) δ 13.2 (s, 1H), 9.09 (s, 1H), 8.88 (s, 1H), 8.65 (s, 1H), 8.15 (d, J=8.8 Hz, 2H), 7.56 (d, J=8.4 Hz, 2H), 7.52-7.45 (m, 2H), 7.39-7.33 (m, 2H), 7.19 (d, J=8.0 Hz, 1H), 7.04 (t, J=73.6 Hz, 1H), 2.61 (s, 3H).

### Example 99: Synthesis of N-(6-(4-Chlorophenyl)thiazolo[4,5-c]pyridin-2-yl)-4-(6-methoxyimidazo[1,5-a]pyridin-7-yl)-6-methylnicotinamide

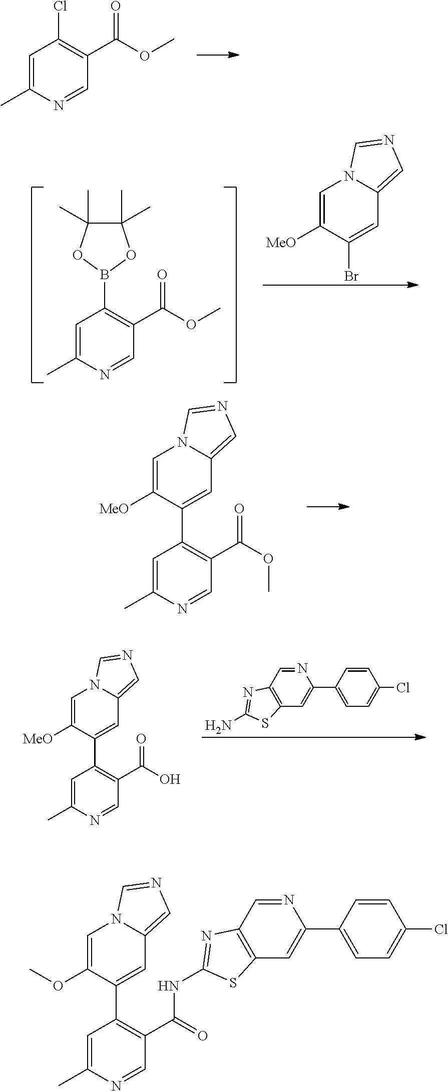

Step 1: A mixture of methyl 4-chloro-6-methylnicotinate (540 mg, 2.91 mmol), Pin₂B₂ (1.48 g, 5.82 mmol), KOAc (857 mg, 8.73 mmol) and Pd(dppf)Cl₂ (213 mg, 0.29 mmol) in dry 1,4-dioxane (30 mL) was heated at 100° C. overnight. After cooling to room temperature, 7-bromo-6-methoxyimidazo[1,5-a]pyridine (180 mg, 0.79 mmol), Na₂CO₃ (252.1 mg, 2.38 mmol), Pd(dppf)Cl₂ (174 mg, 0.24 mmol) and water (6 mL) were added, and the mixture was heated at 100° C. overnight. The mixture was filtered, and the filtrate concentrated under reduced pressure. The residue was purified by reverse phase column (23% ACN in water with 0.1% formic acid modifier) and prep-TLC (DCM/MeOH=12/1, v/v) to give methyl 4-(6-methoxyimidazo[1,5-a]pyridin-7-yl)-6-methylnicotinate (160 mg, 68%) as a yellow solid. LCMS (ESI, m/z): 298 [M+H]⁺.

Step 2: A mixture of methyl 4-(6-methoxyimidazo[1,5-a]pyridin-7-yl)-6-methylnicotinate (50 mg, 0.17 mmol) and NaOH (20 mg, 0.50 mmol) in MeOH (1.2 mL) and water (1.2 mL) was heated at 50° C. for 3 h. The mixture was cooled to RT, adjusted to pH 3-4 with 6 M aqueous HCl and concentrated under reduced pressure. The residue was dissolved in DCM/MeOH (15/1, 5 mL), filtered and the filtrate concentrated under reduced pressure to give 4-(6-methoxyimidazo[1,5-a]pyridin-7-yl)-6-methylnicotinic acid (50 mg) as a yellow solid, which was used directly in the next step. LCMS (ESI, m/z): 284 [M+H]⁺.

Step 3: A mixture of 4-(6-methoxyimidazo[1,5-a]pyridin-7-yl)-6-methylnicotinic acid (50 mg), HATU (77 mg, 0.20 mmol) and DIPEA (66 mg, 0.51 mmol) in NMP (1 mL) was stirred at RT for 2 h. 6-(4-Chlorophenyl)thiazolo[4,5-c]pyridin-2-amine (44 mg, 0.17 mmol) was added and the mixture was heated at 80° C. overnight. After cooling to RT, the mixture was purified by prep-HPLC and prep-TLC (DCM/MeOH=20/1, v/v) to give N-(6-(4-chlorophenyl)thiazolo[4,5-c]pyridin-2-yl)-4-(6-methoxyimidazo[1,5-a]pyridin-7-yl)-6-methylnicotinamide (5 mg, 6% over two steps) as a yellow solid. LCMS (ESI, m/z): 527 [M+H]⁺, ¹H NMR (400 MHz, DMSO-d₆): δ 13.2 (s, 1H), 9.10 (s, 1H), 8.81 (s, 1H), 8.67 (s, 1H), 8.32 (s, 1H), 8.15 (d, J=8.2 Hz, 2H), 8.02 (s, 1H), 7.72 (s, 1H), 7.56 (d, J=8.2 Hz, 2H), 7.48 (d, J=3.0 Hz, 2H), 3.46 (s, 3H), 2.61 (s, 3H).

TABLE O
The following examples were prepared using a similar procedure to that described for Example 99

| Ex. | | | |
|---|---|---|---|
| No. | Structure | Compound Name | Characterization Data |
| 100 | 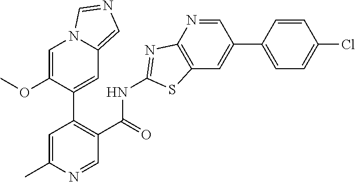 [SMILES: COc1cn2cncc2cc1-c1cc(C)ncc1C(=O)Nc1nc2ncc(-c3ccc(Cl)cc3)cc2s1] | N-(6-(4-Chlorophenyl)- thiazolo[4,5-b]pyridin- 2-yl)-4-(6-methoxyimid- azo[1,5-a]pyridin-7-yl)- 6-methylnicotinamide | LCMS (m/z): 527 [M + H]⁺. ¹H NMR (400 MHz, DMSO- d₆) δ 13.1 (s, 1H), 8.86 (d, J = 2.4 Hz, 1H), 8.82 (s, 1H), 8.73 (d, J = 2.4 Hz, 1H), 8.28 (s, 1H), 8.01 (s, 1H), 7.84- 7.77 (m, 2H), 7.69 (s, 1H), 7.60-7.55 (m, 2H), 7.46 (s, 2H), 3.46 (s, 3H), 2.60 (s, 3H) |
| 101 | 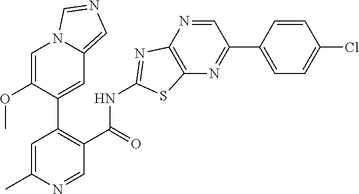 [SMILES: COc1cn2cncc2cc1-c1cc(C)ncc1C(=O)Nc1nc2ncc(-c3ccc(Cl)cc3)nc2s1] | N-(6-(4-Chlorophenyl)- thiazolo[4,5-b]pyrazin- 2-yl)-4-(6-methoxyimid- azo[1,5-a]pyridin-7-yl)- 6-methylnicotinamide | LCMS (m/z): 528 [M + H]⁺. ¹H NMR (400 MHz, DMSO- d₆) δ 13.5 (s, 1H), 9.26 (s, 1H), 8.82 (s, 1H), 8.30 (s, 1H), 8.20 (d, J = 8.6 Hz, 2H), 8.02 (s, 1H), 7.73 (s, 1H), 7.61 (d, J = 8.8 Hz, 2H), 7.48 (d, J = 3.4 Hz, 2H), 3.49 (s, 3H), 2.61 (s, 3H) |

### Example 102: Synthesis of N-(6-(4-Chlorophenyl)thiazolo[4,5-b]pyridin-2-yl)-2′-methoxy-5-methyl-[1,1′-biphenyl]-2-carboxamide

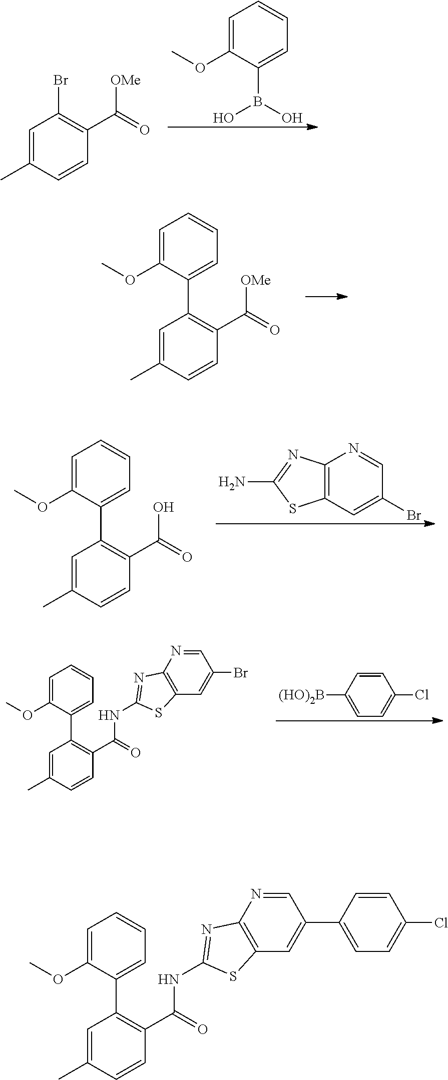

Step 1: To a solution of methyl 2-bromo-4-methylbenzoate (1.00 g, 4.37 mmol) in 1,4-dioxane (4 mL) and water (1 mL) was added (2-methoxyphenyl)boronic acid (995 mg, 6.55 mmol), Pd(PPh₃)₄ (504 mg, 0.43 mmol) and Na₂CO₃ (1.39 g, 13.0 mmol) and the mixture was heated at 100° C. overnight. The mixture was diluted with water (30 mL), extracted with EtOAc (2×30 mL) and the combined organic phases washed with water (30 mL), brine (30 mL), dried over Na₂SO₄, filtered and concentrated under reduced pressure. The residue was purified by silica gel chromatography (Pet. ether/EtOAc=20/1, v/v) to afford methyl 2′-methoxy-5-methyl-[1,1′-biphenyl]-2-carboxylate (960 mg, 85%) as a yellow solid. LCMS (ESI, m/z): 257 [M+H], ¹H NMR (400 MHz, DMSO-d₆): δ 7.65 (d, J=7.8 Hz, 1H), 7.31 (dd, J=8.0, 1.6 Hz, 1H), 7.27-7.23 (m, 1H), 7.19 (dd, J=7.4, 1.6 Hz, 1H), 7.12 (s, 1H), 7.03-6.96 (m, 2H), 3.62 (s, 3H), 3.55 (s, 3H), 2.38 (s, 3H).

Step 2: To a solution of methyl 2′-methoxy-5-methyl-[1,1′-biphenyl]-2-carboxylate (960 mg, 3.75 mmol) in THF (5 mL) and water (5 mL) was added NaOH (450 mg, 11.2 mmol) and the mixture was heated at 50° C. overnight. The mixture was concentrated under reduced pressure to remove most of the organic solvent and the aqueous residue was adjusted to pH 5 with 2 M aqueous HCl, diluted with water (30 mL) and extracted with EtOAc (2×30 mL). The combined organic phases were washed with water (30 mL), brine (30 mL), dried over Na₂SO₄, filtered and concentrated under reduced pressure to give 2′-methoxy-5-methyl-[1,1′-biphenyl]-2-carboxylic acid (780 mg, 85%) as a yellow solid. LCMS (ESI, m/z): 241 [M−H]⁻, ¹H NMR (400 MHz, DMSO-d₆): δ 12.2 (s, 1H), 7.67 (d, J=7.8 Hz, 1H), 7.33-7.26 (m, 1H), 7.21 (dd, J=8.0, 1.6 Hz, 1H), 7.15 (dd, J=7.6, 1.8 Hz, 1H), 7.06 (d, J=1.6 Hz, 1H), 6.97 (ddd, J=8.4, 3.8, 2.8 Hz, 2H), 3.64 (s, 3H), 2.36 (s, 3H).

Step 3: To a solution of 2′-methoxy-5-methyl-[1,1′-biphenyl]-2-carboxylic acid (200 mg, 0.82 mmol) in DMF (5 mL) was added 6-bromothiazolo[4,5-b]pyridin-2-amine (285 mg, 1.24 mmol), HATU (470 mg, 1.24 mmol) and DIPEA (533 mg, 4.13 mmol) and the mixture was heated at 80° C. overnight. The mixture was diluted with water (30 mL), extracted with EtOAc (2×30 mL) and the combined organic phases washed with water (30 mL), brine (30 mL), dried over Na₂SO₄, filtered and concentrated under reduced pressure. The residue was purified by prep-TLC (DCM/MeOH=40/1, v/v) to give N-(6-bromothiazolo[4,5-b]pyridin-2-yl)-2′-methoxy-5-methyl-[1,1′-biphenyl]-2-carboxamide (110 mg, 29%) as a yellow solid. LCMS (ESI, m/z): 454, 456 [M+H]⁺, ¹H NMR (400 MHz, DMSO-d₆): δ 12.9 (s, 1H), 8.70 (d, J=2.2 Hz, 1H), 8.61 (d, J=2.2 Hz, 1H), 7.61 (d, J=7.8 Hz, 1H), 7.34-7.28 (m, 3H), 7.21 (s, 1H), 7.04 (t, J=7.6 Hz, 1H), 6.91 (d, J=8.0 Hz, 1H), 3.46 (s, 3H), 2.42 (s, 3H).

Step 4: A mixture of N-(6-bromothiazolo[4,5-b]pyridin-2-yl)-2′-methoxy-5-methyl-[1,1′-biphenyl]-2-carboxamide (105 mg, 0.23 mmol), (4-chlorophenyl)boronic acid (54 mg, 0.34 mmol), Pd(PPh₃)₄ (27 mg, 0.023 mmol) and Cs₂CO₃ (225 mg, 0.69 mmol) in 1,4-dioxane (2 mL) and water (0.5 mL) was heated at 130° C. under microwave irradiation for 2 h. The mixture was diluted with water (20 mL), extracted with EtOAc (2×20 mL) and the combined organic phases washed with water (20 mL), brine (20 mL), dried over Na₂SO₄, filtered and concentrated under reduced pressure. The residue was purified by prep-HPLC to give N-(6-(4-chlorophenyl)thiazolo[4,5-b]pyridin-2-yl)-2′-methoxy-5-methyl-[1,1′-biphenyl]-2-carboxamide (40 mg, 35%) as a white solid. LCMS (ESI, m/z): 486 [M+H]⁺, ¹H NMR (400 MHz, DMSO-d₆): δ 12.8 (s, 1H), 8.85 (d, J=2.2 Hz, 1H), 8.72 (d, J=2.2 Hz, 1H), 7.83-7.77 (m, 2H), 7.63 (d, J=7.8 Hz, 1H), 7.60-7.55 (m, 2H), 7.35-7.29 (m, 3H), 7.22 (d, J=1.6 Hz, 1H), 7.05 (t, J=7.4 Hz, 1H), 6.91 (d, J=8.0 Hz, 1H), 3.48 (s, 3H), 2.42 (s, 3H).

### Example 103: Synthesis of N-(6-(3,4-Dihydro-2H-pyran-6-yl)thiazolo[4,5-b]pyridin-2-yl)-4-(2-methoxyphenyl)-6-methylnicotinamide

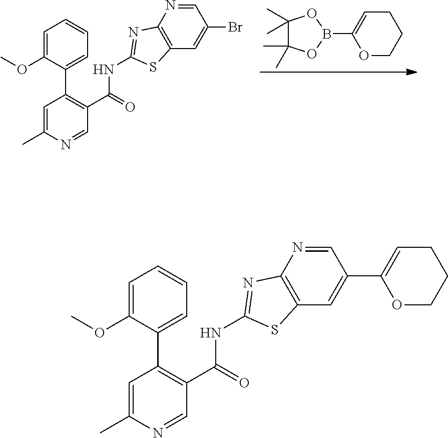

To a solution of N-(6-bromothiazolo[4,5-b]pyridin-2-yl)-4-(2-methoxyphenyl)-6-methylnicotinamide (200 mg, 0.439 mmol) in 1,4-dioxane (4 mL) and water (1 mL) was added 2-(3,4-dihydro-2H-pyran-6-yl)-4,4,5,5-tetramethyl-1,3,2-dioxaborolane (138 mg, 0.658 mmol), Pd(dppf)Cl₂·DCM (36 mg, 0.043 mmol) and Cs₂CO₃ (429 mg, 1.32 mmol) and the mixture was heated at 110° C. in a sealed tube overnight. The mixture was diluted with water, extracted with EtOAc and the combined organic phases washed with water, brine, dried over Na₂SO₄, filtered and concentrated under reduced pressure. The residue was purified by prep-TLC (DCM/MeOH=25/1 to 20/1, v/v) to afford N-(6-(3,4-dihydro-2H-pyran-6-yl)thiazolo[4,5-b]pyridin-2-yl)-4-(2-methoxyphenyl)-6-methylnicotinamide (60 mg, 30%) as a yellow oil. LCMS (ESI, m/z): 459 [M+H]⁺, ¹H NMR (400 MHz, DMSO-d₆): δ 13.0 (s, 1H) 8.73 (d, J=2.4 Hz, 1H), 8.72 (s, 1H), 8.49 (d, J=2.0 Hz, 1H), 7.42-7.36 (m, 2H), 7.32 (s, 1H), 7.09 (t, J=7.4 Hz, 1H), 6.97 (d, J=8.6 Hz, 1H), 5.58 (t, J=4.0 Hz, 1H), 4.20-4.13 (m, 2H), 3.49 (s, 3H), 2.58 (s, 3H), 2.24-2.17 (m, 2H), 1.91-1.83 (m, 2H).

### Example 104: Synthesis of (Racemic)-4-(2-Methoxyphenyl)-6-methyl-N-(6-(tetrahydro-2H-pyran-2-yl)thiazolo[4,5-b]pyridin-2-yl)nicotinamide

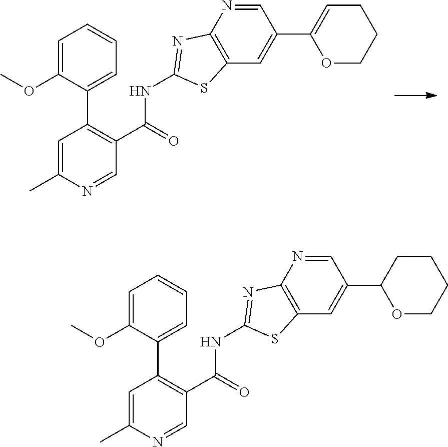 [SMILES: COc1ccccc1-c1cc(C)ncc1C(=O)Nc1nc2ncc(C3=CCCCO3)cc2s1 ; COc1ccccc1-c1cc(C)ncc1C(=O)Nc1nc2ncc(C3CCCCO3)cc2s1]

To a solution of N-(6-(3,4-dihydro-2H-pyran-6-yl)thiazolo[4,5-b]pyridin-2-yl)-4-(2-methoxyphenyl)-6-methylnicotinamide (100 mg, 0.218 mmol) in MeOH (6 mL) was added 40% Pd(OH)₂/C (40 mg) and the mixture was heated at 50° C. under a H₂ atmosphere (2.8 MPa) overnight. After filtration, the filtrate was concentrated and the residue purified by prep-HPLC to afford (Racemic)-4-(2-methoxyphenyl)-6-methyl-N-(6-(tetrahydro-2H-pyran-2-yl)thiazolo[4,5-b]pyridin-2-yl)nicotinamide (25 mg, 25%) as a white solid. LCMS (ESI, m/z): 461 [M+H]⁺, ¹H NMR (400 MHz, DMSO-d₆): δ 13.0 (s, 1H), 8.72 (s, 1H), 8.51 (d, J=2.0 Hz, 1H), 8.37 (d, J=2.0 Hz, 1H), 7.43-7.35 (m, 2H), 7.33 (s, 1H), 7.09 (t, J=7.4 Hz, 1H), 6.96 (d, J=8.4 Hz, 1H), 4.49 (dd, J=11.0, 2.0 Hz, 1H), 4.05 (d, J=11.6 Hz, 1H), 3.62-3.56 (m, 1H), 3.48 (s, 3H), 2.58 (s, 3H), 1.86 (d, J=12.4 Hz, 2H), 1.58 (d, J=4.8 Hz, 4H).

### Example 105: Synthesis of (R or S)-4-(2-methoxyphenyl)-6-methyl-N-(6-(tetrahydro-2H-pyran-2-yl)thiazolo[4,5-b]pyridin-2-yl)nicotinamide

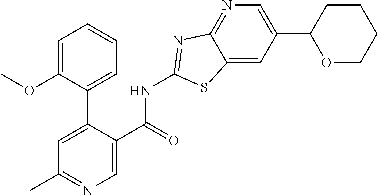 [SMILES: COc1ccccc1-c1cc(C)ncc1C(=O)Nc1nc2ncc(C3CCCCO3)cc2s1]

(Racemic)-4-(2-methoxyphenyl)-6-methyl-N-(6-(tetrahydro-2H-pyran-2-yl)thiazolo[4,5-b]pyridin-2-yl)nicotinamide (Example 104) was purified by chiral SFC (Column: Daicel Chiralpak, OJ 20*250 mm, 10 um; Mobile Phase: 35% MeOH (0.2% NH₃ modifier), 65% carbon dioxide; Flow rate: 100 mL/min) to afford two peaks:

Peak 1, retention time 2.01 min. LCMS (ESI, m/z): 461 [M+H]⁺, ¹H NMR (MeOD, 400 MHz) δ 8.696 (s, 1H), 8.50 (d, J=2.0 Hz, 1H), 8.35 (d, J=2.0 Hz, 1H), 7.44-7.39 (m, 3H), 7.10 (t, J=8.0 Hz, 1H), 6.93 (d, J=8.4 Hz, 1H), 4.54 (d, J=10.0 Hz, 1H), 4.15-4.11 (m, 1H), 3.71-3.56 (m, 1H), 3.57 (s, 3H), 2.65 (s, 3H), 1.99-1.90 (m, 2H), 1.80-1.60 (m, 4H)

### Example 106: Synthesis of (S or R)-4-(2-methoxyphenyl)-6-methyl-N-(6-(tetrahydro-2H-pyran-2-yl)thiazolo[4,5-b]pyridin-2-yl)nicotinamide

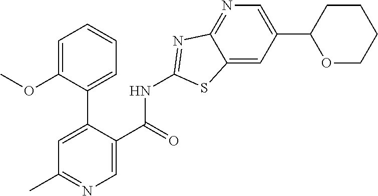 [SMILES: COc1ccccc1-c1cc(C)ncc1C(=O)Nc1nc2ncc(C3CCCCO3)cc2s1]

Peak 2, retention time 2.62 min. LCMS (ESI, m/z): 461 [M+H]⁺, ¹H NMR (MeOD, 400 MHz) δ 8.70 (s, 1H), 8.50 (d, J=2.0 Hz, 1H), 8.35 (d, J=2.0 Hz, 1H), 7.44-7.39 (m, 3H), 7.10 (t, J=8.0 Hz, 1H), 6.93 (d, J=8.4 Hz, 1H), 4.54 (d, J=10.0 Hz, 1H), 4.15-4.11 (m, 1H), 3.71-3.56 (m, 1H), 3.57 (s, 3H), 2.65 (s, 3H), 1.99-1.90 (m, 2H), 1.80-1.60 (m, 4H)

### Example 107: Synthesis of N-(6-(4-Chlorophenyl)thiazolo[4,5-b]pyridin-2-yl)-7-(2-methoxyphenyl)imidazo[1,2-a]pyridine-6-carboxamide

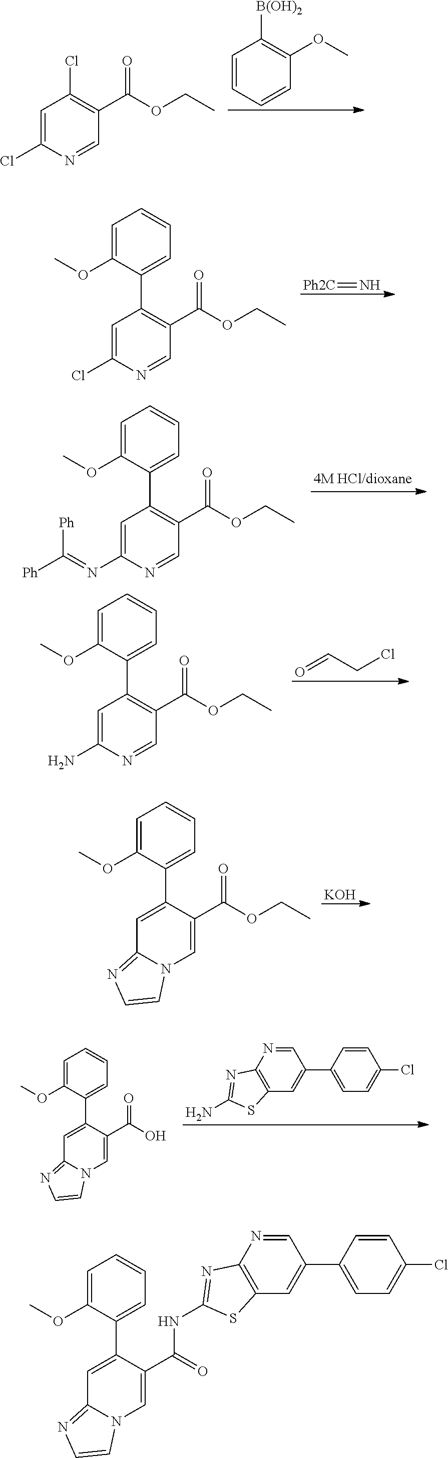

Step 1: To a solution of ethyl 4,6-dichloronicotinate (5 g, 23 mmol), (2-methoxyphenyl)boronic acid (3.45 g, 22.7 mmol) and K₂CO₃ (9.42 g, 68.2 mmol) in 1,4-dioxane (80 mL) and water (20 mL) was added Pd(dppf)Cl₂ (1.65 g, 2.27 mmol) and the mixture was heated at 80° C. for 8 h. The mixture was diluted with water, extracted with DCM (30 mL×5) and the combined organic layers dried over Na₂SO₄, filtered and concentrated under reduced pressure. The residue was purified by silica gel chromatography (Pet.

Ether/EtOAc=100/1 to 50/1, v/v) and prep-TLC (Pet. Ether/EtOAc=30/1, v/v) to afford ethyl 6-chloro-4-(2-methoxyphenyl)nicotinate (1.5 g, 235) as a yellow oil. LCMS (ESI, m/z): 292 [M+H](.

Step 2: To a solution of ethyl 6-chloro-4-(2-methoxyphenyl)nicotinate (1.2 g, 4.1 mmol), benzophenone imine (1.12 g, 6.17 mmol), Xantphos (476 mg, 0.823 mmol) and Cs₂CO₃ (4.04 g, 12.3 mmol) in 1,4-dioxane (48 mL) was added Pd₂(dba)₃ (376 mg, 0.411 mmol) and the mixture was heated at 110° C. overnight. The mixture was diluted with water, extracted with DCM (25 m×5), dried over Na₂SO₄, filtered and concentrated under reduced pressure. The residue was purified by silica gel chromatography (Pet. Ether/EtOAc=3/1, v/v) to afford ethyl 6-((diphenylmethylene)amino)-4-(2-methoxyphenyl)nicotinate (900 mg, 501) as a yellow oil. LCMS (ESI, m/z): 437 [M+H]⁺.

Step 3: A mixture of ethyl 6-((diphenylmethylene)amino)-4-(2-methoxyphenyl)nicotinate (900 mg, 2.06 mmol) and 4 M HCl/dioxane (5 m) was stirred at room temperature for 2 h. The mixture was concentrated under reduced pressure and the residue was diluted with a saturated aqueous NaHCO₃ solution (20 mL), extracted with DCM (15 mL×3) and the combined organic phases dried over Na₂SO₄, filtered and concentrated under reduced pressure to afford ethyl 6-amino-4-(2-methoxyphenyl)nicotinate (1.3 g) as a yellow solid, which was used directly in the next step. LCMS (ESI, m/z): 273 [M+H]+

Step 4: A mixture of ethyl 6-amino-4-(2-methoxyphenyl)nicotinate (1.3 g), 2-chloroacetaldehyde (3.75 g, 47.5 mmol) and NaHCO₃ (1.60 g, 19.1 mmol) in DMF (20 mL) was heated at 120° C. overnight. The mixture was diluted with water, extracted with EtOAc (20 mL×6) and the combined organic phases washed with brine (30 mL×3), dried over Na₂SO₄, filtered and concentrated under reduced pressure. The residue was purified by prep-TLC (DCM/MeOH=50/1, v/v) to afford ethyl 7-(2-methoxyphenyl)imidazo[1,2-a]pyridine-6-carboxylate (198 mg, 32% over two steps) as a yellow oil. LCMS (ESI, m/z): 297 [M+H]⁺, ¹H NMR (400 MHz, DMSO-d₆): δ 9.10 (d, J=0.8 Hz, 1H), 8.08 (s, 1H), 7.67 (d, J=1.2 Hz, 1H), 7.42-7.32 (m, 3H), 7.08-6.98 (m, 2H), 4.06 (q, J=7.0 Hz, 2H), 3.65 (s, 3H), 1.03 (t, J=7.0 Hz, 3H).

Step 5: To a solution of ethyl 7-(2-methoxyphenyl)imidazo[1,2-a]pyridine-6-carboxylate (257 mg, 0.866 mmol) in MeOH (4 mL) and water (1 mL) was added KOH (146 mg, 2.60 mmol) and the mixture was heated at 80° C. for 1 h. The mixture was adjusted to pH 3 with 6 M aqueous HCl and concentrated under reduced pressure. The residue was dissolved in DCM/MeOH (10/1, 15 mL), filtered and the filtrate concentrated under reduced pressure to afford 7-(2-methoxyphenyl)imidazo[1,2-a]pyridine-6-carboxylic acid (260 mg) as a yellow solid. LCMS (ESI, m/z): 269.10 [M+H], ¹H NMR (400 MHz, DMSO-d₆): δ 13.3 (s, 1H), 9.36 (s, 1H), 8.34 (d, J=1.8 Hz, 1H), 8.16 (d, J=2.0 Hz, 1H), 7.74 (s, 1H), 7.49-7.37 (m, 2H), 7.14-7.05 (m, 2H), 3.70 (s, 3H).

Step 6: To a solution of 7-(2-methoxyphenyl)imidazo[1,2-a]pyridine-6-carboxylic acid (55 mg, 0.205 mmol) and HATU (117 mg, 0.308 mmol) in NMP (2 mL) was added DIPEA (79.5 mg, 0.615 mmol) and the mixture was stirred at room temperature for 2 h. 6-(4-Chlorophenyl)thiazolo[4,5-b]pyridin-2-amine (64.4 mg, 0.246 mmol) was added and the mixture was heated at 80° C. overnight. The mixture was diluted with water, extracted with EtOAc (15 mL×5) and the combined organic phases washed with brine (10 mL×3), dried over Na₂SO₄, filtered and concentrated under reduced pressure. The residue was purified by prep-HPLC to afford N-(6-(4-chlorophenyl)thiazolo[4,5-b]pyridin-2-yl)-7-(2-methoxyphenyl)imidazo[1,2-a]pyridine-6-carboxamide (14.4 mg, 14%) as a yellow solid. LCMS (ESI, m/z): 512 [M+H]⁺, ¹H NMR (400 MHz, DMSO-d₆): δ 13.4 (s, 1H), 9.36 (s, 1H), 8.89 (d, J=2.2 Hz, 1H), 8.77 (d, J=2.2 Hz, 1H), 8.38 (d, J=1.8 Hz, 1H), 8.14 (d, J=1.8 Hz, 1H), 7.82 (s, 1H), 7.81 (d, J=8.6 Hz, 2H), 7.59 (d, J=8.4 Hz, 2H), 7.53 (dd, J=7.6, 1.6 Hz, 1H), 7.45 (td, J=7.8, 1.6 Hz, 1H), 7.16 (t, J=7.4 Hz, 1H), 7.01 (d, J=8.2 Hz, 1H), 3.51 (s, 3H).

### Example 108: Synthesis of 4-(6-Methoxy-1-oxoisoindolin-5-yl)-6-methyl-N-(6-(2-methylpyridin-4-yl)thiazolo[4,5-b]pyridin-2-yl)nicotinamide

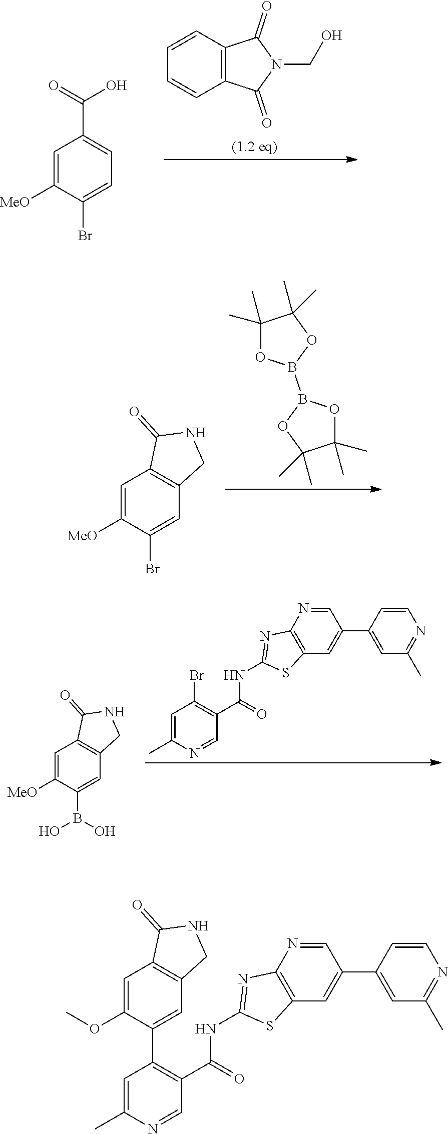

Step 1: A mixture of 4-bromo-3-methoxybenzoic acid (2.0 g, 8.7 mmol) and 2-(hydroxymethyl)isoindoline-1,3-dione (1.84 g, 10.4 mmol) in conc. H₂SO₄ (8.5 mL) was heated at 80° C. for 3 h. After cooling to room temperature, the mixture was slowly poured into ice-water (20 g) and the resulting precipitate collected by filtration. The filter cake was added into a mixture of EtOH (17 mL) and concentrated aqueous NH₄OH ammonium hydroxide (17 mL) and the mixture was heated at 80° C. for 3 h. After cooling to room temperature, the precipitate was collected by filtration and dried under reduced pressure to afford 5-bromo-6-methoxyisoindolin-1-one (850 mg, 40%) as an off-white solid. LCMS (ESI, m/z): 242, 244 [M+H]⁺, ¹H NMR (400 MHz, DMSO-d₆): δ 8.60 (s, 1H), 7.83 (s, 1H), 7.29 (s, 1H), 4.29 (s, 2H), 3.92 (s, 3H).

Step 2: To a solution of 5-bromo-6-methoxyisoindolin-1-one (300 mg, 1.24 mmol), bis(pinacolato)diboron (629 mg, 2.48 mmol) and potassium acetate (365 mg, 3.5 mmol) in dioxane (5 mL) under a N2 atmosphere was added Pd(dppf)Cl₂ (91 mg, 0.124 mmol) and the mixture was heated at 100° C. overnight. The mixture was concentrated under reduced pressure and the residue was diluted with water (30 mL) and extracted with EtOAc (30 mL×2). The combined organic layers were washed with water (30 mL), brine (30 mL), dried over Na₂SO₄, filtered and concentrated under reduced pressure. The residue was purified by reverse phase column chromatography (35% ACN/water with 0.1% formic acid modifier) to afford (6-methoxy-1-oxoisoindolin-5-yl)boronic acid (150 mg, 50%) as an off-white solid. LCMS (ESI, m/z): 208 [M+H]⁺.

Step 3: To a solution of 4-bromo-6-methyl-N-(6-(2-methylpyridin-4-yl)thiazolo[4,5-b]pyridin-2-yl)nicotinamide (50 mg, 0.11 mmol), (6-methoxy-1-oxoisoindolin-5-yl)boronic acid (29 mg, 0.14 mmol) and Na₂CO₃ (37 mg, 0.34 mmol) in 1,4-dioxane (2 mL) and water (0.4 mL) under a N2 atmosphere was added Pd(PPh₃)₄ (9 mg, 0.011 mmol) and the mixture was heated at 130° C. under microwave irradiation for 2 h. The mixture was diluted with a 10/1 mixture of DCM/MeOH, filtered and the filtrate concentrated under reduced pressure. The residue was purified by prep-TLC (DCM/MeOH=12/1, v/v) to afford 4-(6-methoxy-6-oxoisoindolin-5-yl)-6-methyl-N-(6-(2-methylpyridin-4-yl)thiazolo[4,5-b]pyridin-2-yl)nicotinamide (20 mg, 33%) as an off-white solid. LCMS (ESI, m/z): 523 [M+H]⁺, ¹H NMR (400 MHz, DMSO-d₆): δ 13.2 (s, 1H), 8.98 (d, J=2.2 Hz, 1H), 8.86 (d, J=2.2 Hz, 1H), 8.81 (s, 1H), 8.61 (s, 1H), 8.54 (d, J=5.2 Hz, 1H), 7.71 (s, 1H), 7.62 (d, J=3.6 Hz, 2H), 7.39 (s, 1H), 7.17 (s, 1H), 4.38 (s, 2H), 3.56 (s, 3H), 2.61 (s, 3H), 2.56 (s, 3H).

TABLE P
The following examples were prepared using a similar procedure to that described for Example 108

| Ex. | | | |
|---|---|---|---|
| No. | Structure | Compound Name | Characterization Data |
| 109 | 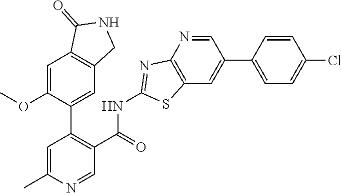 [SMILES: COc1cc2c(cc1-c1cc(C)ncc1C(=O)Nc1nc3ncc(-c4ccc(Cl)cc4)cc3s1)CNC2=O] | N-(6-(4-Chlorophenyl)- thiazolo[4,5-b]pyridin- 2-yl)-4-(6-methoxy-1- oxoisoindolin-5-yl)-6- methylnicotinamide | LCMS (m/z): 542 [M + H]⁺. ¹H NMR (400 MHz, DMSO- d₆) δ 13.2 (s, 1H), 8.87 (d, J = 2.2 Hz, 1H), 8.80 (s, 1H), 8.74 (d, J = 2.2 Hz, 1H), 8.63 (s, 1H), 7.84-7.74 (m, 2H), 7.65-7.56 (m, 3H), 7.39 (s, 1H), 7.17 (s, 1H), 4.38 (s, 2H), 3.55 (s, 3H), 2.61 (s, 3H) |
| 110 | 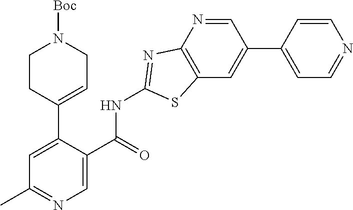 [SMILES: Cc1cc(C2=CCN(C(=O)OC(C)(C)C)CC2)c(C(=O)Nc2nc3ncc(-c4ccncc4)cc3s2)cn1] | tert-Butyl 2′-methyl- 5′-((6-(pyridin-4-yl)- thiazolo[4,5-b]pyridin- 2-yl)carbamoyl)-3,6- dihydro-[4,4′-bipyr- idine]-1(2H)-carbox- ylate | LCMS (m/z): 529 [M + H]⁺. ¹H NMR (400 MHz, DMSO- d₆) δ 13.3 (s, 1H), 9.03 (d, J = 2.4 Hz, 1H), 8.96 (d, J = 2.4 Hz, 1H), 8.72-8.68 (m, 3H), 7.89-7.81 (m, 2H), 7.31 (s, 1H), 5.83 (s, 1H), 3.88 (d, J = 3.2 Hz, 2H), 3.46 (t, J = 5.6 Hz, 2H), 2.55 (s, 3H), 2.36 (br s, 2H), 1.33 (s, 9H) |

### Example 111: Synthesis of N-(6-Bromothiazolo[4,5-b]pyridin-2-yl)-4-(2,4-dimethoxyphenyl)-6-methylnicotinamide

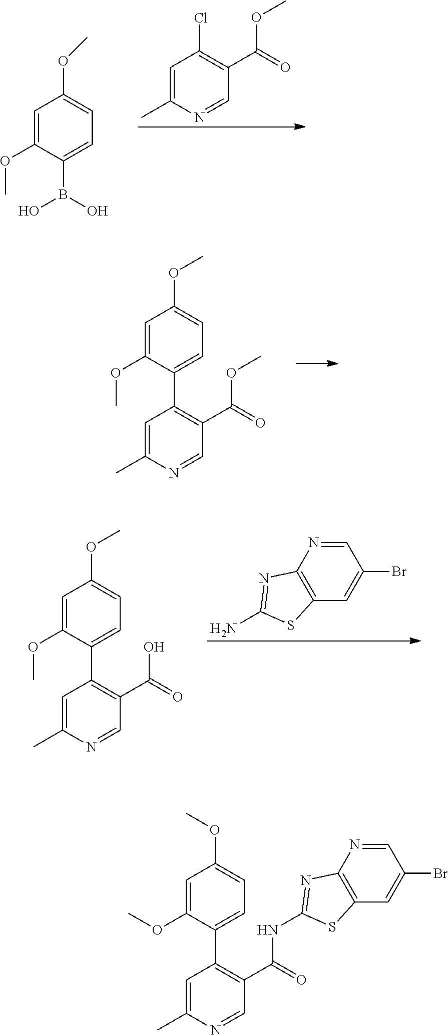

Step 1: To a solution of (2,4-dimethoxyphenyl)boronic acid (1.0 g, 5.5 mmol) in 1,4-dioxane (15 mL) was added methyl 4-chloro-6-methylnicotinate (1.02 g, 5.50 mmol), Pd(PPh₃)₄ (317.5 g, 0.274 mmol) and Cs₂CO₃ (5.37 g, 16.5 mmol) and the mixture was heated at 85° C. overnight. The mixture was diluted with water (40 mL), extracted with EtOAc (40 mL×3) and the combined organic layers washed with brine, dried over Na₂SO₄, filtered and concentrated under reduced pressure. The residue was purified by silica gel chromatography (Pet.Ether/EtOAc=10/1, v/v) to afford methyl 4-(2,4-dimethoxyphenyl)-6-methylnicotinate (853 mg, 78%) as a white solid. LCMS (ESI, m/z): 288 [M+H]⁺, ¹H NMR (400 MHz, DMSO-d₆): δ 8.68 (s, 1H), 7.26-7.19 (m, 2H), 6.66-6.57 (m, 2H), 3.82 (s, 3H), 3.65 (s, 3H), 3.64 (s, 3H), 2.53 (s, 3H).

Step 2: To a solution of methyl 4-(2,4-dimethoxyphenyl)-6-methylnicotinate (853 mg, 2.97 mmol) in MeOH (6 mL) and H₂O (1 mL) was added NaOH (356 mg, 8.91 mmol) and the mixture was heated at 50° C. for 3 h. The mixture was concentrated under reduced pressure and the residue adjusted to pH 4-5 with 2 M aqueous HCl. The resulting precipitate was collected by filtration and dried to afford 4-(2,4-dimethoxyphenyl)-6-methylnicotinic acid (416 mg, 51%) as a white solid. LCMS (ESI, m/z): 274 [M+H]⁺.

Step 3: To a solution of 4-(2,4-dimethoxyphenyl)-6-methylnicotinic acid (50 mg, 0.183 mmol) in DMF (3 mL) was added HOBt (37.1 mg, 0.274 mmol), EDCI·HCl (52.6 mg, 0.274 mmol) and TEA (55.5 mg, 0.548 mmol) and the mixture was stirred at room temperature for 2 h. 6-Bromothiazolo[4,5-b]pyridin-2-amine (42.1 mg, 0.183 mmol) was added and the mixture was stirred at room temperature overnight. The mixture was diluted with water (40 mL), extracted with EtOAc (40 mL×3) and the combined organic layers were washed with brine, dried over Na₂SO₄, filtered and concentrated under reduced pressure. The residue was purified by prep-TLC (DCM/MeOH=20/1, v/v) to afford N-(6-bromothiazolo[4,5-b]pyridin-2-yl)-4-(2,4-dimethoxyphenyl)-6-methylnicotinamide (34 mg, 39%) as a solid. LCMS (ESI, m/z): 485, 487 [M+H]⁺, ¹H NMR (400 MHz, DMSO-d₆): δ 13.1 (s, 1H), 8.73 (d, J=2.2 Hz, 1H), 8.68 (s, 1H), 8.63 (d, J=2.2 Hz, 1H), 7.37-7.29 (m, 2H), 6.68 (dd, J=8.4, 2.2 Hz, 1H), 6.51 (d, J=2.2 Hz, 1H), 3.79 (s, 3H), 3.46 (s, 3H), 2.57 (s, 3H).

TABLE Q
The following examples were prepared using a similar procedure to that described for Example 111

| Ex. | | | |
|---|---|---|---|
| No. | Structure | Compound Name | Characterization Data |
| 112 | 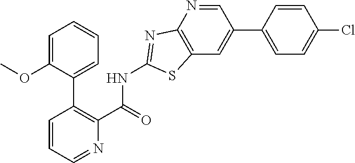 [SMILES: COc1ccccc1-c1cccnc1C(=O)Nc1nc2ncc(-c3ccc(Cl)cc3)cc2s1] | N-(6-(4-Chlorophenyl)- thiazolo[4,5-b]pyridin- 2-yl)-3-(2-methoxy- phenyl)picolinamide | LCMS (m/z): 473 [M + H]⁺. ¹H NMR (400 MHz, DMSO- d₆) δ 13.0 (s, 1H), 8.89 (d, J = 2.4 Hz, 1H), 8.75 (d, J = 2.4 Hz, 1H), 8.70 (dd, J = 4.8, 1.6 Hz, 1H), 7.91 (d, J = 1.6 Hz, 1H), 7.81 (d, J = 8.6 Hz, 2H), 7.73 (dd, J = 7.8, 4.8 Hz, 1H), 7.58 (d, J = 8.6 Hz, 2H), 7.42- 7.33 (m, 2H), 7.09 (t, J = 7.6 |
| | | | Hz, 1H), 7.01 (d, J = 8.2 Hz, |
| | | | 1H), 3.53 (s, 3H) |
| 113 | 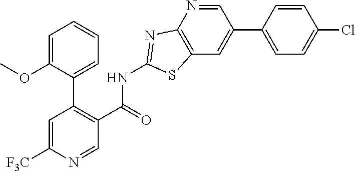 [SMILES: COc1ccccc1-c1cc(C(F)(F)F)ncc1C(=O)Nc1nc2ncc(-c3ccc(Cl)cc3)cc2s1] | N-(6-(4-Chlorophenyl)- thiazolo[4,5-b]pyridin- 2-yl)-4-(2-methoxy- phenyl)-6-(trifluoro- methyl)nicotinamide | LCMS (m/z): 541 [M + H]⁺. ¹H NMR (400 MHz, DMSO- d₆) δ 13.0 (s, 1H), 8.89 (d, J = 2.4 Hz, 1H), 8.75 (d, J = 2.4 Hz, 1H), 8.70 (dd, J = 4.8, 1.6 Hz, 1H), 7.91 (d, J = 1.6 Hz, 1H), 7.81 (d, J = 8.6 Hz, 2H), 7.73 (dd, J = 7.8, 4.8 Hz, 1H), 7.58 (d, J = 8.6 Hz, 2H), 7.42- 7.33 (m, 2H), 7.09 (t, J = 7.6 Hz, 1H), 7.01 (d, J = 8.2 Hz, |
| | | | 1H), 3.53 (s, 3H) |
| 114 | 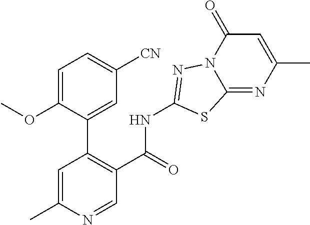 | 4-(5-cyano-2-methoxy- phenyl)-6-methyl-N- (7-methyl-5-oxo-5H- [1,3,4]thiadiazolo[3,2- a]pyrimidin-2-yl)- nicotinamide | LCMS (m/z): 433 [M + H]⁺. ¹H NMR (400 MHz, DMSO- d₆) δ 13.55 (s, 1H), 8.79 (s, 1H), 8.06-7.81 (m, 2H), 7.43 (s, 1H), 7.20 (d, J = 8.6 Hz, 1H), 6.24 (s, 1H), 3.65 (s, 3H), 2.59 (s, 3H), 2.28 (s, 3H) |

### Example 115: Synthesis of N-(6-(3-Cyanopyridin-2-yl)thiazolo[4,5-b]pyridin-2-yl)-4-(2-methoxyphenyl)-6-methylnicotinamide

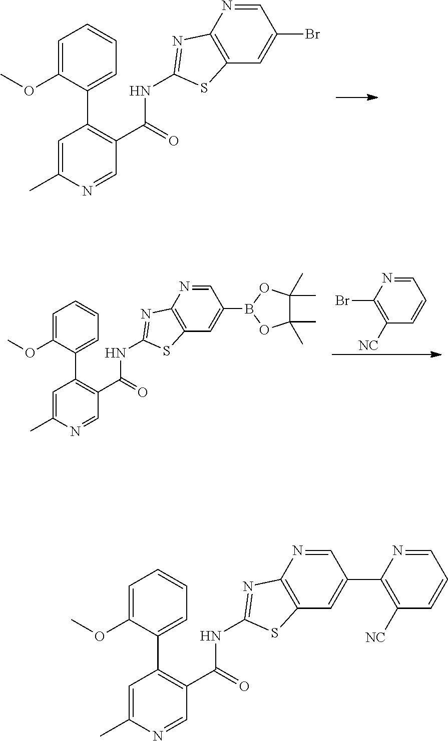

Step 1: To a solution of N-(6-bromothiazolo[4,5-b]pyridin-2-yl)-4-(2-methoxyphenyl)-6-methylnicotinamide (50 mg, 0.11 mmol) and Pin₂B₂ (285 mg, 1.11 mmol) in 1,4-dioxane (3 mL) was added Pd(dppf)Cl₂ (16 mg, 0.02 mmol) and CH₃COOK (32 mg, 0.32 mmol) and the mixture was heated at 100° C. overnight under a N2 atmosphere. The mixture was filtered and the filtrate containing 4-(2-methoxyphenyl)-6-methyl-N-(6-(4,4,5,5-tetramethyl-1,3,2-dioxaborolan-2-yl)thiazolo[4,5-b]pyridin-2-yl)nicotinamide was used in the next step directly. LCMS (ESI, m/z): 503 [M+H]⁺.

Step 2: To the filtrate containing 4-(2-methoxyphenyl)-6-methyl-N-(6-(4,4,5,5-tetramethyl-1,3,2-dioxaborolan-2-yl)thiazolo[4,5-b]pyridin-2-yl)nicotinamide obtained from step 1 was added water (1 mL), 2-bromonicotinonitrile (18 mg, 0.10 mmol), Pd(dppf)Cl₂ (14 mg, 0.02 mmol) and K₂CO₃ (30 mg, 0.32 mmol) and the mixture was heated at 100° C. overnight. The mixture was diluted with water (50 mL), extracted with EtOAc (50 mL×3) and the combined organic layers washed with brine (30 mL), dried over Na₂SO₄, filtered and concentrated under reduced pressure. The residue was purified by prep-HPLC to afford N-(6-(3-cyanopyridin-2-yl)thiazolo[4,5-b]pyridin-2-yl)-4-(2-methoxyphenyl)-6-methylnicotinamide (16 mg, 34%) as a white solid. LCMS (ESI, m/z): 479 [M+H]⁺, ¹H NMR (400 MHz, DMSO-d₆): δ 13.1 (s, 1H), 9.02 (d, J=2.4 Hz, 1H), 8.98 (dd, J=4.8, 1.6 Hz, 1H), 8.88 (d, J=1.8 Hz, 1H), 8.78 (s, 1H), 8.46 (dd, J=7.8, 1.8 Hz, 1H), 7.65 (dd, J=7.8, 4.8 Hz, 1H), 7.42-7.36 (m, 2H), 7.31 (s, 1H), 7.09 (t, J=7.4 Hz, 1H), 6.98 (d, J=8.6 Hz, 1H), 3.54 (s, 3H), 2.60 (s, 3H).

### Example 116: Synthesis of 4-(2-Methoxyphenyl)-6-methyl-N-(6-(3-methylpyridin-2-yl)thiazolo[4,5-b]pyridin-2-yl)nicotinamide

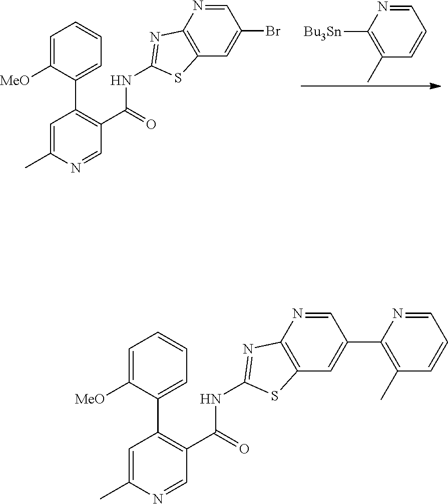

To a solution of N-(6-bromothiazolo[4,5-b]pyridin-2-yl)-4-(2-methoxyphenyl)-6-methylnicotinamide (100 mg, 0.22 mmol) in DMF (3 mL) was added CuI (8 mg, 0.04 mmol), LiCl (2 mg, 0.04 mmol), Pd(PPh₃)₄ (5 mg, 0.004 mmol) and 3-methyl-2-(tributylstannyl)pyridine (386 mg, 0.99 mmol) and the mixture was heated at 130° C. under microwave irradiation for 2 h. The mixture was diluted with water (20 mL), extracted with EtOAc (20 mL×4) and the combined organic layers washed with brine (20 mL), dried over Na₂SO₄ and concentrated under reduced pressure. The residue was purified by prep-HPLC to afford 4-(2-methoxyphenyl)-6-methyl-N-(6-(3-methylpyridin-2-yl)thiazolo[4,5-b]pyridin-2-yl)nicotinamide (47 mg, 46%) as a white solid. LCMS (ESI, m/z): 468 [M+H]⁺, ¹H NMR (400 MHz, DMSO-d₆): δ 13.2 (s, 1H), 8.87-8.74 (m, 2H), 8.70 (s, 1H), 8.61 (s, 1H), 7.95 (d, J=7.6 Hz, 1H), 7.52-7.40 (m, 4H), 7.12 (t, J=7.4 Hz, 1H), 6.99 (d, J=8.3 Hz, 1H), 3.51 (s, 3H), 2.63 (s, 3H), 2.42 (s, 3H).

### Example 117: Synthesis of 4-(2-Methoxyphenyl)-6-methyl-N-(thiazolo[5,4-c]pyridin-2-yl)nicotinamide

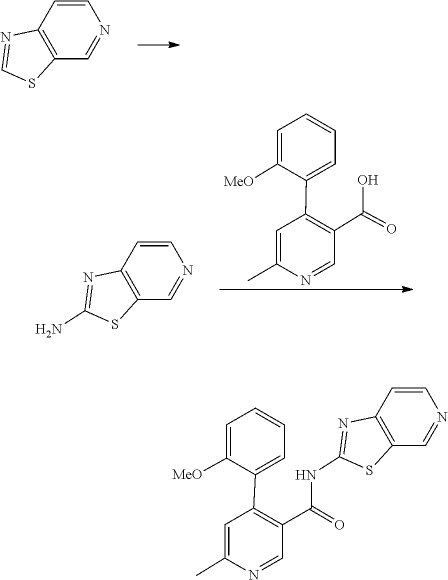 [SMILES: c1cc2ncsc2cn1 ; Nc1nc2ccncc2s1 ; COc1ccccc1-c1cc(C)ncc1C(=O)O ; COc1ccccc1-c1cc(C)ncc1C(=O)Nc1nc2ccncc2s1]

Step 1: To a solution of thiazolo[5,4-c]pyridine (30 mg, 0.22 mmol), (diacetoxyiodo)benzene (107 mg, 0.33 mmol) and trifluoromethanesulfonic anhydride (50 mg, 0.176 mmol) in water (2 mL) was added KSeCN (48 mg, 0.33 mmol) and 12 (11 mg, 0.044 mmol) and the mixture was heated at 110° C. in a sealed tube for 6 h. The mixture was filtered and the filtrate was concentrated under reduced pressure. The residue was purified by prep-TLC (DCM/MeOH=8/1, v/v) to give thiazolo[5,4-c]pyridin-2-amine (30 mg, 90%) as an off-white solid. LCMS (ESI, m/z): 152 [M+H]⁺, ¹H NMR (400 MHz, DMSO-d₆): δ 8.83 (s, 1H), 8.38 (s, 2H), 8.33 (d, J=5.8 Hz, 1H), 7.36 (d, J=5.8 Hz, 1H).

Step 2: To a solution of thiazolo[5,4-c]pyridin-2-amine (112 mg, 0.74 mmol) and 4-(2-methoxyphenyl)-6-methylnicotinic acid (150 mg, 0.617 mmol) in DCM (5 mL) was added EDCI·HCl (178 mg, 0.925 mmol) and DMAP (113 mg, 0.925 mmol) and the mixture was stirred at room temperature overnight. The mixture was diluted with water (30 mL), extracted with DCM (2×30 mL) and the combined organic layers washed with water (30 mL), brine (30 mL), dried over Na₂SO₄, filtered and concentrated under reduced pressure. The residue was purified by prep-TLC (DCM/MeOH=15/1, v/v) to afford 4-(2-methoxyphenyl)-6-methyl-N-(thiazolo[5,4-c]pyridin-2-yl)nicotinamide (36.5 mg, 38%) as a white solid. LCMS (ESI, m/z): 377 [M+H], ¹H NMR (400 MHz, DMSO-d₆): δ 13.2 (s, 1H), 9.20 (s, 1H), 8.74 (s, 1H), 8.55 (d, J=5.6 Hz, 1H), 7.76 (d, J=5.6 Hz, 1H), 7.42-7.36 (m, 2H), 7.35 (s, 1H), 7.10 (t, J=7.4 Hz, 1H), 6.96 (d, J=8.4 Hz, 1H), 3.47 (s, 3H), 2.59 (s, 3H).

### Example 118: Synthesis of 4-(2-Methoxyphenyl)-6-methyl-N-(6-(trifluoromethyl)thiazolo[4,5-b]pyridin-2-yl)nicotinamide

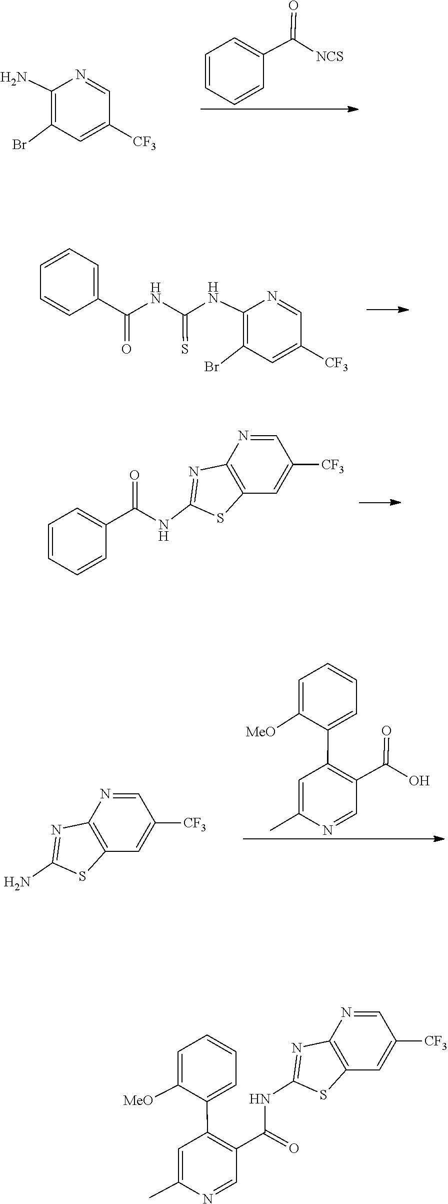

Step 1: To a solution of 3-bromo-5-(trifluoromethyl)pyridin-2-amine (500 mg, 2.05 mmol) in acetone (6 mL) was added benzoyl isothiocyanate (507 mg, 3.11 mmol) and the mixture was heated at 55° C. overnight. The mixture was diluted with water (50 mL), extracted with EtOAc (50 mL×3) and the combined organic layers washed with brine (30 mL), dried over Na₂SO₄, filtered and concentrated under reduced pressure. The residue was purified by silica gel chromatography (Pet. ether/EtOAc=10/1 to 5/1, v/v) to afford N-((3-bromo-5-(trifluoromethyl)pyridin-2-yl)carbamothioyl)benzamide (510 mg, 61%) as a yellow solid. LCMS (ESI, m/z): 404 [M+H]⁺, ¹H NMR (400 MHz, DMSO-d₆): δ 12.6 (s, 1H), 12.0 (s, 1H), 8.95 (dd, J=2.2, 1.2 Hz, 1H), 8.73 (d, J=2.2 Hz, 1H), 8.06-7.97 (m, 2H), 7.73-7.65 (m, 1H), 7.56 (t, J=7.6 Hz, 2H).

Step 2: To a solution of N-((3-bromo-5-(trifluoromethyl)pyridin-2-yl)carbamothioyl)benzamide (500 mg, 1.24 mmol) in NMP (5 mL) was added MeONa (334 mg, 6.16 mmol) and the mixture was heated at 120° C. under a N2 atmosphere for 5 h. The mixture was diluted with water (50 mL), extracted with EtOAc (50 mL×3) and the combined organic layers washed with brine (30 mL), dried over Na₂SO₄, filtered and concentrated under reduced pressure. The residue was purified by silica gel chromatography (Pet. ether/EtOAc=5/1 to 2/1, v/v) to afford N-(6-(trifluoromethyl)thiazolo[4,5-b]pyridin-2-yl)benzamide (290 mg, 73%) as a yellow solid. LCMS (ESI, m/z): 324 [M+H]⁺, ¹H NMR (400 MHz, DMSO-d₆): δ 13.4 (s, 1H), 8.97-8.81 (m, 2H), 8.21-8.15 (m, 2H), 7.72-7.63 (m, 1H), 7.58 (dd, J=8.2, 6.8 Hz, 2H).

Step 3: A solution of N-(6-(trifluoromethyl)thiazolo[4,5-b]pyridin-2-yl)benzamide (140 mg, 0.43 mmol) in H₂SO₄ (2 mL) was heated at 120° C. for 3 h. The mixture was diluted with ice-water (2 mL) and adjusted to pH 8-9 with 2 M aqueous NaOH solution. The resulting precipitate was collected by filtration to afford 6-(trifluoromethyl)thiazolo[4,5-b]pyridin-2-amine (61 mg, 60%) as a white solid. LCMS (ESI, m/z): 220 [M+H]⁺, ¹H NMR (400 MHz, DMSO-d₆): δ 8.55 (d, J=2.4 Hz, 1H), 8.48 (d, J=2.4 Hz, 1H), 8.39 (s, 2H).

Step 4: To a solution of 4-(2-methoxyphenyl)-6-methylnicotinic acid (50 mg, 0.20 mmol) in DMF (3 mL) was added EDCI·HCl (43 mg, 0.24 mmol), HOBt (31 mg, 0.24 mmol) and TEA (64 mg, 0.60 mmol) and the mixture was stirred at room temperature for 1 h. 6-(Trifluoromethyl)thiazolo[4,5-b]pyridin-2-amine (45 mg, 0.20 mmol) was added and the mixture was heated at 80° C. overnight. The mixture was diluted with water (30 mL), extracted with EtOAc (30 mL×3) and the combined organic layers washed with brine (30 mL×2), dried over Na₂SO₄, filtered and concentrated under reduced pressure. The residue was purified by prep-HPLC to afford 4-(2-methoxyphenyl)-6-methyl-N-(6-(trifluoromethyl)thiazolo[4,5-b]pyridin-2-yl)nicotinamide (10 mg, 11%) as a white solid. LCMS (ESI, m/z): 445 [M+H]⁺, ¹H NMR (400 MHz, DMSO-d₆): δ 13.3 (s, 1H), 8.97-8.89 (m, 2H), 8.75 (s, 1H), 7.40 (t, J=8.0 Hz, 2H), 7.35 (s, 1H), 7.10 (t, J=7.6 Hz, 1H), 6.97 (d, J=8.2 Hz, 1H), 3.48 (s, 3H), 2.59 (s, 3H).

### Example 119: Synthesis of N-(6-Cyclopropylthiazolo[4,5-b]pyridin-2-yl)-4-(2-methoxyphenyl)-6-methylnicotinamide

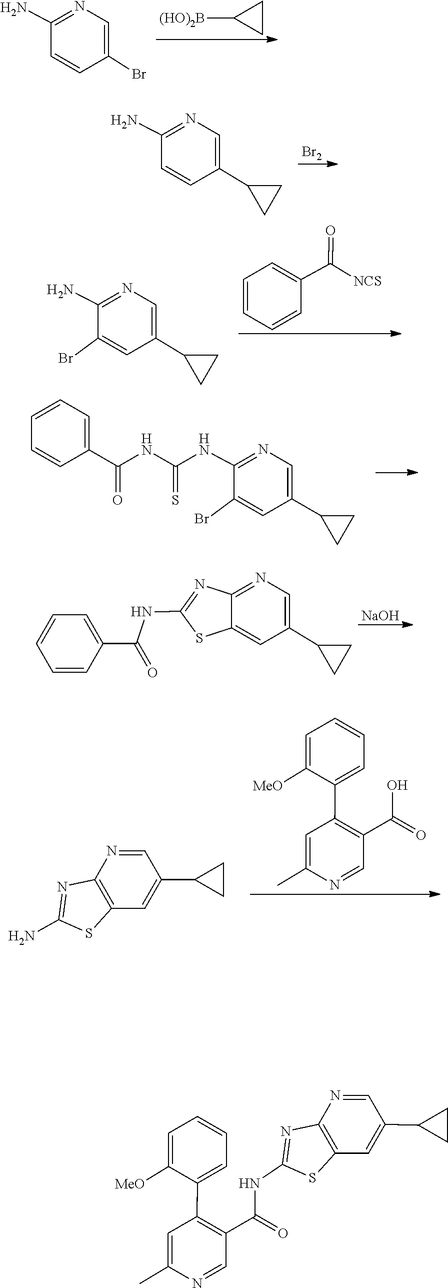

Step 1: A mixture of 5-bromopyridin-2-amine (5 g, 29 mmol), cyclopropylboronic acid (7.45 g, 86.7 mmol), Pd(OAc)₂ (650 mg, 2.89 mmol), PCy₃ (1.62 g, 5.78 mmol) and K₃PO₄ (24.54 g, 115.6 mmol) in toluene (70 mL) and water (7 mL) was heated at 80° C. overnight under N2. The mixture was diluted with water (30 mL), extracted with EtOAc (50 mL×3) and the combined organic layers washed with brine (20 mL), dried over Na₂SO₄, filtered and concentrated under reduced pressure. The residue was purified by silica gel chromatography (Pet. Ether/EtOAc=50/1 to 2/1, v/v) to afford 5-cyclopropylpyridin-2-amine (3.86 g, 99%) as a yellow oil. LCMS (ESI, m/z): 135 [M+H]⁺, ¹H NMR (400 MHz, DMSO-d₆): δ 7.74 (d, J=2.4 Hz, 1H), 7.04 (dd, J=8.6, 2.5 Hz, 1H), 6.36 (d, J=8.4 Hz, 1H), 5.61 (s, 2H), 1.78-1.69 (m, 1H), 0.90-0.73 (m, 2H), 0.57-0.43 (m, 2H).

Step 2: To a solution of 5-cyclopropylpyridin-2-amine (1.34 g, 9.99 mmol) in DCM (5 mL) was added Br₂ (1.6 g, 9.99 mmol) and the mixture was stirred at room temperature overnight. The mixture was diluted with water (20 mL) and extracted with DCM (50 mL×3). The combined organic layers were washed with brine (20 mL), dried over Na₂SO₄, filtered and concentrated under reduced pressure. The residue was purified by silica gel chromatography (Pet. Ether/EtOAc=50/1 to 10/1) to afford 3-bromo-5-cyclopropylpyridin-2-amine (108 mg, 34%) as a yellow oil. LCMS (ESI, m/z): 213, 215 [M+H]⁺, ¹H NMR (400 MHz, DMSO-d₆): δ 7.79 (d, J=2.2 Hz, 1H), 7.39 (d, J=2.2 Hz, 1H), 5.91 (s, 2H), 1.81-1.74 (m, 1H), 0.86-0.79 (m, 2H), 0.60-0.55 (m, 2H).

Step 3: To a solution of 3-bromo-5-cyclopropylpyridin-2-amine (800 mg, 3.75 mmol) in 1,4-dioxane (10 mL) was added benzoyl isothiocyanate (674 mg, 4.13 mmol) and the mixture was stirred at room temperature for 6 h. The mixture was diluted with water (20 mL), extracted with EtOAc (50 mL×2) and the combined organic layers washed with brine (20 mL), dried over Na₂SO₄, filtered and concentrated under reduced pressure. The residue was purified by silica gel chromatography (Pet. Ether/EtOAc=15/1 to DCM/MeOH=10/1) to afford N-((3-bromo-5-cyclopropylpyridin-2-yl)carbamothioyl)benzamide (1.14 g, 80%) as a yellow solid. LCMS (ESI, m/z): 376 [M+H]⁺, ¹H NMR (400 MHz, DMSO-d₆): δ 12.3 (s, 1H), 11.8 (s, 1H), 8.32 (d, J=2.2 Hz, 1H), 8.06-7.96 (m, 2H), 7.84 (d, J=2.2 Hz, 1H), 7.71-7.63 (m, 1H), 7.55 (t, J=7.6 Hz, 2H), 2.06-1.97 (m, 1H), 1.10-0.98 (m, 2H), 0.90-0.78 (m, 2H).

Step 4: To a solution of N-((3-bromo-5-cyclopropylpyridin-2-yl)carbamothioyl) benzamide (50 mg, 0.13 mmol) in DME (2 mL) was added Pd(PPh₃)₄ (15.4 mg, 0.013 mmol) and Cs₂CO₃ (86.6 mg, 0.265 mmol) and the mixture was heated at 80° C. overnight under N2. The mixture was diluted with water (10 mL), extracted with DCM (20 mL×2) and the combined organic layers washed with brine, dried over Na₂SO₄, filtered and concentrated under reduced pressure. The residue was purified by prep-TLC (DCM/MeOH=20/1, v/v) to afford N-(6-cyclopropylthiazolo[4,5-b]pyridin-2-yl) benzamide (20.3 mg, 51%) as a yellow solid. LCMS (ESI, m/z): 296 [M+H]⁺.

Step 5: To a solution of N-(6-cyclopropylthiazolo[4,5-b]pyridin-2-yl)benzamide (310 mg, 1.05 mmol) in MeOH (15 mL) and water (5 mL) was added NaOH (252 mg, 6.3 mmol) and the mixture was heated at 85° C. overnight. The mixture was diluted with water (20 mL), extracted with EtOAc (30 mL×3) and the combined organic layers washed with brine, dried over Na₂SO₄, filtered and concentrated under reduced pressure. The residue was purified by prep-TLC (DCM/MeOH=20/1, v/v) to afford 6-cyclopropylthiazolo[4,5-b]pyridin-2-amine (52 mg, 25%) as a white solid. LCMS (ESI, m/z): 192 [M+H]⁺, ¹H NMR (400 MHz, DMSO-d₆): δ 8.07 (d, J=2.2 Hz, 1H), 7.77 (s, 2H), 7.70 (d, J=2.2 Hz, 1H), 1.98-1.89 (m, 1H), 0.99-0.88 (m, 2H), 0.69-0.64 (m, 2H).

Step 6: To a solution of 4-(2-methoxyphenyl)-6-methylnicotinic acid (53 mg, 0.21 mmol) in NMP (3 mL) was added EDCI·HCl (62.7 mg, 0.326 mmol), HOBt (44.2 mg, 0.326 mmol) and DIPEA (84.5 mg, 0.653 mmol) and the mixture was stirred at room temperature for 6 h. 6-Cyclopropylthiazolo[4,5-b]pyridin-2-amine (50 mg, 0.261 mmol) was added and the mixture was heated at 80° C. overnight. The mixture was diluted with water (10 mL), extracted with EtOAc (20 mL×3) and the combined organic layers washed with brine, dried over Na₂SO₄, filtered and concentrated under reduced pressure. The residue was purified by prep-HPLC to afford N-(6-cyclopropylthiazolo[4,5-b]pyridin-2-yl)-4-(2-methoxyphenyl)-6-methylnicotinamide (1.9 mg, 4%) as a white solid. LCMS (ESI, m/z): 417 [M+H]⁺, ¹H NMR (400 MHz, DMSO-d₆): δ 8.73 (s, 1H), 8.38 (d, J=2.2 Hz, 1H), 8.00 (s, 1H), 7.41-7.34 (m, 2H), 7.30 (s, 1H), 7.07 (t, J=7.4 Hz, 1H), 6.96 (d, J=8.2 Hz, 1H), 3.48 (s, 3H), 2.57 (s, 3H), 2.10-2.01 (m, 1H), 1.08-0.95 (m, 2H), 0.80-0.72 (m, 2H).

### Example 120: Synthesis of 4-(2-Methoxyphenyl)-6-methyl-N-(6-(2-oxopiperidin-1-yl)benzo[d]thiazol-2-yl)nicotinamide

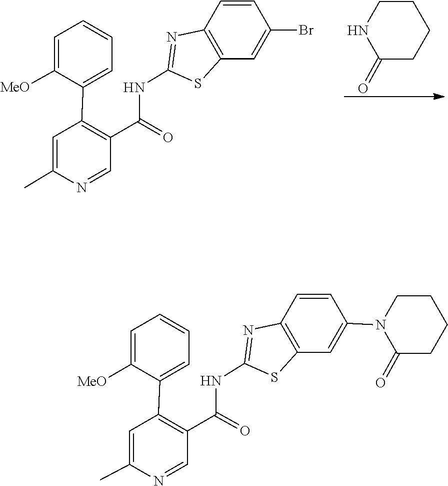 [SMILES: COc1ccccc1-c1cc(C)ncc1C(=O)Nc1nc2ccc(Br)cc2s1 ; O=C1CCCCN1 ; COc1ccccc1-c1cc(C)ncc1C(=O)Nc1nc2ccc(N3CCCCC3=O)cc2s1]

To a solution of N-(6-bromobenzo[d]thiazol-2-yl)-4-(2-methoxyphenyl)-6-methylnicotinamide (300 mg, 0.66 mmol) in dry DMF (7.5 mL) was added piperidin-2-one (196 mg, 1.98 mmol), Cs₂CO₃ (430 mg, 1.32 mmol), CuI (252 mg, 1.32 mmol) and N,N′-Dimethyl-1,2-ethanediamine (232 mg, 2.64 mmol) and the mixture was heated at 110° C. for 16 h. After cooling to room temperature, the mixture was diluted with water (5 mL), extracted with EtOAc (3×20 mL) and the combined organic phases were dried over Na₂SO₄, filtered and concentrated under reduced pressure. The residue was purified by prep-HPLC to give 4-(2-methoxyphenyl)-6-methyl-N-(6-(2-oxopiperidin-1-yl)benzo[d]thiazol-2-yl)nicotinamide (4.0 mg, 1%) as an off-white solid. LCMS (ESI, m/z): 473 [M+H]⁺, ¹H NMR (400 MHz, DMSO-d₆): δ 12.7 (s, 1H), 8.71 (s, 1H), 7.87-7.83 (m, 1H), 7.75-7.69 (m, 1H), 7.42-7.35 (m, 2H), 7.34-7.28 (m, 2H), 7.08 (t, J=7.2 Hz, 1H), 6.95 (d, J=8.8 Hz, 1H), 3.66-3.60 (m, 2H), 3.48 (s, 3H), 2.58 (s, 3H), 2.43-2.37 (m, 2H), 1.95-1.79 (m, 4H).

TABLE R
The following examples were prepared using a similar procedure to that described
for Example 120

| Ex. | | | |
|---|---|---|---|
| No. | Structure | Compound Name | Characterization Data |
| 121 | 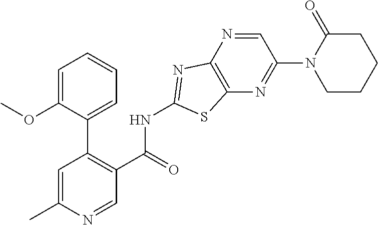 [SMILES: COc1ccccc1-c1cc(C)ncc1C(=O)Nc1nc2ncc(N3CCCCC3=O)nc2s1] | 4-(2- methoxyphenyl)- 6- methyl-N-(6-(2- oxopiperidin-1- yl)thiazolo[4,5-b] pyrazin-2-yl) nicotinamide | LCMS (m/z): 475 [M + H]⁺. ¹H NMR (400 MHz, DMSO-d₆) δ 13.2 (s, 1H), 8.89 (s, 1H), 8.76 (s, 1H), 7.44-7.36 (m, 3H), 7.10 (t, J = 7.6 Hz, 1H), 6.98 (d, J = 8.4 Hz, 1H), 3.87 (t, J = 5.8 Hz, 2H), 3.51 (s, 3H), 2.60 (s, 3H), 2.52 (t, J = 6.8 Hz, 2H), 1.95- 1.82 (m, 4H) |

### Example 122: Synthesis of 6-Methyl-N-(6-(pyridin-4-yl)thiazolo[4,5-b]pyridin-2-yl)-4-(4-oxa-7-azaspiro[2.5]octan-7-yl)nicotinamide

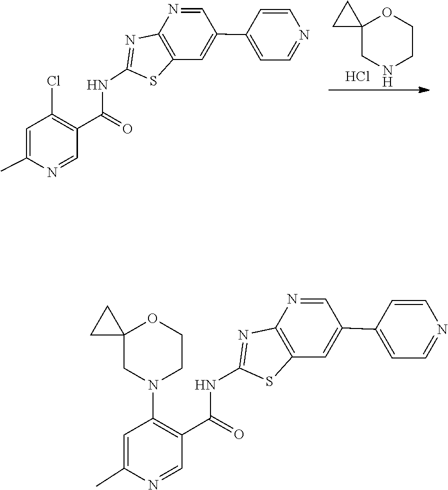

To a solution of 4-oxa-7-azaspiro[2.5]octane hydrochloride (8 mg, 0.05 mmol) and DIPEA (20 mg, 0.15 mmol) in ACN (1.5 mL) and DMF (1.5 mL) was added 4-chloro-6-methyl-N-(6-(pyridin-4-yl)thiazolo[4,5-b]pyridin-2-yl)nicotinamide (20 mg, 0.05 mmol) and the mixture was heated at 80° C. overnight. The mixture was diluted with water (5 mL), extracted with EtOAc (10 mL×3) and the combined organic layers washed with brine (6 mL), dried over Na₂SO₄, filtered and concentrated under reduced pressure. The residue was purified by prep-TLC (DCM/MeOH=15/1, v/v) to give 6-methyl-N-(6-(pyridin-4-yl)thiazolo[4,5-b]pyridin-2-yl)-4-(4-oxa-7-azaspiro[2.5]octan-7-yl)nicotinamide (23 mg, 63%) as a yellow solid. LCMS (ESI, m/z): 459 [M+H]⁺, ¹H NMR (400 MHz, DMSO-d₆): δ 13.2 (s, 1H), 9.03 (d, J=2.4 Hz, 1H), 8.96 (d, J=2.4 Hz, 1H), 8.71-8.69 (m, 2H), 8.48 (s, 1H), 7.86-7.84 (m, 2H), 7.00 (s, 1H), 3.79-3.74 (m, 2H), 3.24-3.18 (m, 2H), 3.14 (s, 2H), 2.46 (s, 3H), 0.72-0.67 (m, 2H), 0.60-0.55 (m, 2H).

TABLE S
The following examples were prepared using a similar procedure to that described for Example 122

| Ex. No. | Structure | Compound Name | Characterization Data |
|---|---|---|---|
| 123 | 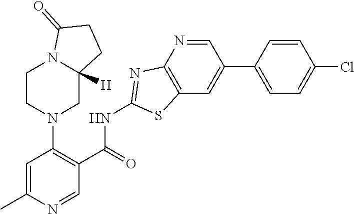 [SMILES: Cc1cc(N2CCN3C(=O)CC[C@@H]3C2)c(C(=O)Nc2nc3ncc(-c4ccc(Cl)cc4)cc3s2)cn1] | (R)-N-(6-(4-Chloro- phenyl)thiazolo[4,5-b] pyridin-2-yl)-6-methyl- 4-(6-oxo-hexahydro- pyrrolo[1,2-a]pyrazin- 2(1H)-yl)nicotinamide | LCMS (m/z): 519 [M + H]⁺. ¹H NMR (400 MHz, DMSO-d₆) δ 13.6 (s, 1H), 8.92 (d, J = 2.2 Hz, 1H), 8.85 (d, J = 2.2 Hz, 1H), 8.70 (s, 1H), 7.83 (d, J = 8.6 Hz, 2H), 7.60 (d, J = 8.4 Hz, 2H), 7.44 (s, 1H), 3.96 (d, J = 12.4 Hz, 1H), 3.89- 3.71 (m, 3H), 3.24-3.14 (m, 1H), 3.12-3.03 (m, 1H), 2.96 (t, J = 11.6 |
| | | | Hz, 1H), 2.55 (s, 3H), |
| | | | 2.35-2.21 (m, 2H), |
| | | | 2.14-2.01 (m, 1H), |
| | | | 1.66-1.48 (m, 1H) |
| 124 | 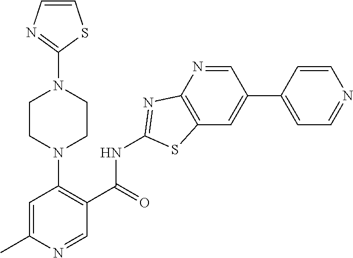 [SMILES: Cc1cc(N2CCN(c3nccs3)CC2)c(C(=O)Nc2nc3ncc(-c4ccncc4)cc3s2)cn1] | 6-Methyl-N-(6-(pyridin- 4-yl)thiazolo[4,5-b]pyr- idin-2-yl)-4-(4-(thiazol- 2-yl)piperazin-1-yl)- nicotinamide | LCMS (m/z): 515 [M + H]⁺. ¹H NMR (400 MHz, DMSO-d₆) δ 13.1 (s, 1H), 9.03-8.99 (m, 1H), 8.98-8.95 (m, 1H), 8.72-8.67 (m, 2H), 8.51 (s, 1H), 7.87-7.83 (m, 2H), 7.17 (d, J = 3.6 Hz, 1H), 7.02 (s, 1H), 6.87 (d, J = 3.6 Hz, 1H), 3.55-3.50 (m, 4H), 3.31-3.27 (m, 4H), 2.47 (s, 3H) |
| 125 | 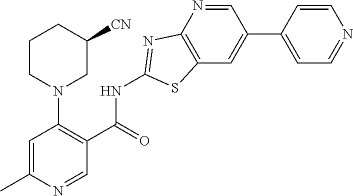 [SMILES: Cc1cc(N2CCC[C@@H](C#N)C2)c(C(=O)Nc2nc3ncc(-c4ccncc4)cc3s2)cn1] | (R)-4-(3-Cyanopiperi- din-1-yl)-6-methyl-N- (6-(pyridin-4-yl)thi- azolo[4,5-b]pyridin-2- yl)nicotinamide | LCMS (m/z): 456 [M + H]⁺. ¹H NMR (400 MHz, DMSO-d₆) δ 9.01 (d, J = 2.4 Hz, 1H), 8.92 (d, J = 2.6 Hz, 1H), 8.71-8.67 (m, 2H), 8.47 (s, 1H), 7.88-7.79 (m, 2H), 7.02 (s, 1H), 3.52 (dd, J = 12.8, 3.6 Hz, 1H), 3.38-3.30 (m, 1H), 3.06-2.84 (m, 2H), 2.81-2.71 (m, 1H), 2.46 |
| | | | (s, 3H), 2.07-1.90 (m, |
| | | | 2H), 1.82-1.53 (m, 2H) |
| 126 | 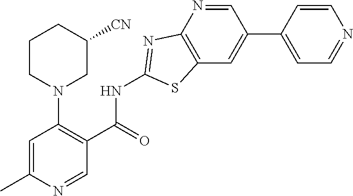 [SMILES: Cc1cc(N2CCC[C@H](C#N)C2)c(C(=O)Nc2nc3ncc(-c4ccncc4)cc3s2)cn1] | (S)-4-(3-Cyanopiperi- din-1-yl)-6-methyl-N- (6-(pyridin-4-yl)thi- azolo[4,5-b]pyridin-2- yl)nicotinamide | LCMS (m/z): 456 [M + H]⁺. ¹H NMR (400 MHz, DMSO-d₆) δ 9.01 (d, J = 2.2 Hz, 1H), 8.93 (d, J = 2.2 Hz, 1H), 8.72-8.66 (m, 2H), 8.47 (s, 1H), 7.86-7.79 (m, 2H), 7.04 (s, 1H), 3.53 (dd, J = 12.8, 3.6 Hz, 1H), 3.40-3.30 (m, 1H), 3.14-3.07 (m, 2H), 2.94-2.84 (m, 1H), 2.47 |
| | | | (s, 3H), 2.08-1.93 (m, |
| | | | 2H), 1.82-1.57 (m, 2H) |
| 127 | 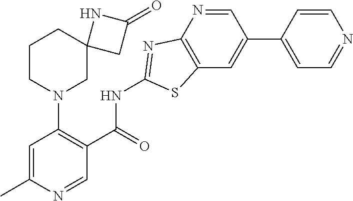 [SMILES: Cc1cc(N2CCCC3(CC(=O)N3)C2)c(C(=O)Nc2nc3ncc(-c4ccncc4)cc3s2)cn1] | (Racemic)-6-Methyl-4- (2-oxo-1,6-diazaspiro [3.5]nonan-6-yl)-N-(6- (pyridin-4-yl)thiazolo [4,5-b]pyridin-2-yl)- nicotinamide | LCMS (m/z): 486 [M + H]⁺. ¹H NMR (400 MHz, DMSO-d₆) δ 13.6 (s, 1H), 9.19 (d, J = 2.2 Hz, 1H), 9.14 (d, J = 2.2 Hz, 1H), 8.91 (d, J = 5.4 Hz, 2H), 8.68 (s, 1H), 8.38 (s, 1H), 8.22 (d, J = 5.0 Hz, 2H), 7.46 (s, 1H), 3.72-3.60 (m, 2H), 3.48 (t, J = 5.4 Hz, 2H), 2.67-2.62 (m, 2H), 2.56 (s, 3H), 1.96-1.89 (m, 1H), 1.79-1.58 (m, 3H) |
| 128 | 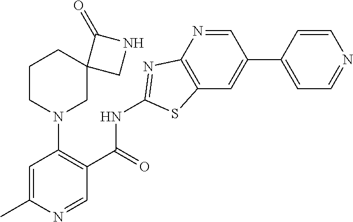 [SMILES: Cc1cc(N2CCCC3(CNC3=O)C2)c(C(=O)Nc2nc3ncc(-c4ccncc4)cc3s2)cn1] | (Racemic)-6-Methyl-4- (1-oxo-2,6-diazaspiro [3.5]nonan-6-yl)-N-(6- (pyridin-4-yl)thiazolo [4,5-b]pyridin-2-yl)- nicotinamide | LCMS (m/z): 486 [M + H]⁺. ¹H NMR (400 MHz, DMSO-d₆) δ 13.0 (s, 1H), 9.00 (d, J = 2.4 Hz, 1H), 8.92 (s, 1H), 8.69 (d, J = 5.2 Hz, 2H), 8.48 (s, 1H), 7.89-7.80 (m, 3H), 7.04 (s, 1H), 3.44 (d, J = 12.2 Hz, 1H), 3.25 (s, 1H), 3.20- 3.11 (m, 2H), 3.06 (d, J = 5.8 Hz, 1H), 2.85 (t, J = 11.6 Hz, 1H), 2.45 (s, 3H), 1.82 (d, J = 12.4 |
| | | | Hz, 1H), 1.75-1.56 (m, |
| | | | 3H) |
| 129 | 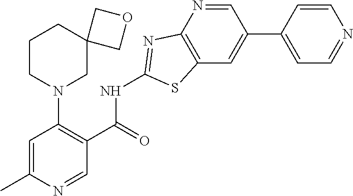 [SMILES: Cc1cc(N2CCCC3(COC3)C2)c(C(=O)Nc2nc3ncc(-c4ccncc4)cc3s2)cn1] | 6-Methyl-N-(6-(pyri- din-4-yl)thiazolo[4,5-b] pyridin-2-yl)-4-(2-oxa- 6-azaspiro[3.5]nonan-6- yl)nicotinamide | LCMS (m/z): 473 [M + H]⁺. ¹H NMR (400 MHz, DMSO-d₆) δ 9.18 (d, J = 2.2 Hz, 1H), 9.13 (d, J = 2.4 Hz, 1H), 8.91 (d, J = 5.8 Hz, 2H), 8.66 (s, 1H), 8.26 (d, J = 6.2 Hz, 2H), 7.47 (s, 1H), 4.28 (d, J = 6.4 Hz, 2H), 4.22 (d, J = 6.6 Hz, 2H), 3.77 (s, 2H), 3.37 (s, 2H), 2.55 (s, 3H), 1.94- |
| | | | 1.88 (m, 2H), 1.58 (br s, |
| | | | 2H). |
| 130 | 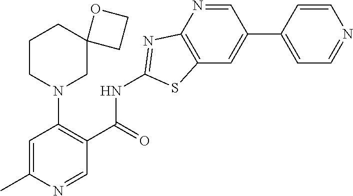 [SMILES: Cc1cc(N2CCCC3(CCO3)C2)c(C(=O)Nc2nc3ncc(-c4ccncc4)cc3s2)cn1] | (Racemic)-6-Methyl-N- (6-(pyridin-4-yl)thiazolo [4,5-b]pyridin-2-yl)-4- (1-oxa-6-azaspiro[3.5] nonan-6-yl)nicotin- amide | LCMS (m/z): 473 [M + H]⁺. ¹H NMR (400 MHz, DMSO-d₆) δ 12.9 (s, 1H), 9.02 (s, 1H), 8.95 (s, 1H), 8.70 (s, 2H), 8.45 (s, 1H), 8.14 (s, 1H), 7.89-7.79 (m, 2H), 7.03 (s, 1H), 4.33 (t, J = 7.8 Hz, 2H), 3.55-3.44 (m, 2H), 3.16- 3.06 (m, 2H), 3.02-2.91 (m, 1H), 2.45 (s, 3H), |
| | | | 2.38-2.28 (m, 1H), 1.95- |
| | | | 1.84 (m, 1H), 1.76-1.63 |
| | | | (m, 2H), 1.52 (br s, 1H) |
| 131 | 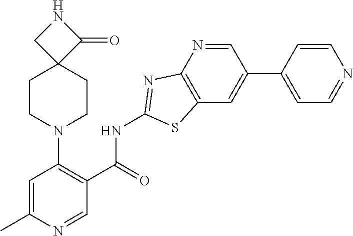 [SMILES: Cc1cc(N2CCC3(CC2)CNC3=O)c(C(=O)Nc2nc3ncc(-c4ccncc4)cc3s2)cn1] | 6-Methyl-4-(1-oxo-2,7- diazaspiro[3.5]nonan-7- yl)-N-(6-(pyridin-4-yl)- thiazolo[4,5-b]pyridin- 2-yl)nicotinamide | LCMS (m/z): 486 [M + H]⁺. ¹H NMR (400 MHz, DMSO-d₆) δ 13.1 (s, 1H), 9.03 (d, J = 2.2 Hz, 1H), 8.96 (d, J = 2.2 Hz, 1H), 8.70 (d, J = 5.2 Hz, 2H), 8.46 (s, 1H), 7.87-7.83 (m, 2H), 7.78 (s, 1H), 7.00 (s, 1H), 3.45-3.36 (m, 2H), 3.17- 3.06 (m, 4H), 2.46 (s, 3H), 1.95-1.75 (m, 4H) |
| 132 | 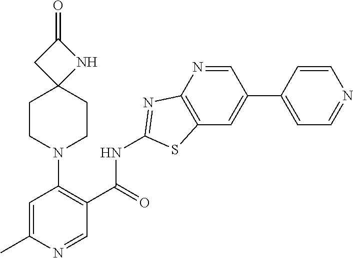 [SMILES: Cc1cc(N2CCC3(CC2)CC(=O)N3)c(C(=O)Nc2nc3ncc(-c4ccncc4)cc3s2)cn1] | 6-Methyl-4-(2-oxo-1,7- diazaspiro[3.5]nonan-7- yl)-N-(6-(pyridin-4-yl)- thiazolo[4,5-b]pyridin- 2-yl)nicotinamide | LCMS (m/z): 486 [M + H]⁺. ¹H NMR (400 MHz, DMSO-d₆) δ 13.1 (s, 1H), 9.03 (d, J = 2.4 Hz, 1H), 8.96 (d, J = 2.4 Hz, 1H), 8.72-8.68 (m, 2H), 8.46 (s, 1H), 8.34 (s, 1H), 7.86-7.83 (m, 2H), 6.98 (s, 1H), 3.26- 3.19 (m, 2H), 3.17-3.08 (m, 2H), 2.63 (s, 2H), 2.45 (s, 3H), 1.85-1.73 (m, 4H) |
| 133 | 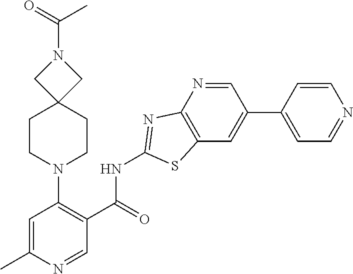 [SMILES: CC(=O)N1CC2(CCN(c3cc(C)ncc3C(=O)Nc3nc4ncc(-c5ccncc5)cc4s3)CC2)C1] | 4-(2-Acetyl-2,7-diaza- spiro[3.5]nonan-7-yl)- 6-methyl-N-(6-(pyridin- 4-yl)thiazolo[4,5-b]pyr- idin-2-yl)nicotinamide | LCMS (m/z): 514 [M + H]⁺. ¹H NMR (400 MHz, DMSO-d₆) δ 12.8 (s, 1H), 9.01 (d, J = 2.2 Hz, 1H), 8.93 (d, J = 2.2 Hz, 1H), 8.74-8.65 (m, 2H), 8.49 (s, 1H), 8.13 (s, 1H), 7.89-7.78 (m, 2H), 6.99 (s, 1H), 3.84 (s, 2H), 3.57 (s, 2H), 3.12 (t, J = 5.4 Hz, 4H), 2.46 (s, 3H), 1.83 (t, J = 5.6 Hz, 4H), 1.74 (s, 3H) |
| 134 | 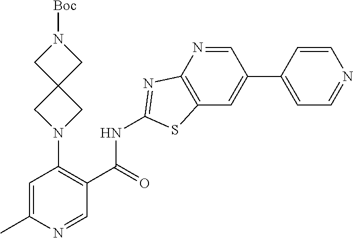 [SMILES: Cc1cc(N2CC3(CN(C(=O)OC(C)(C)C)C3)C2)c(C(=O)Nc2nc3ncc(-c4ccncc4)cc3s2)cn1] | tert-Butyl 6-(2-methyl- 5-((6-(pyridin-4-yl)- thiazolo[4,5-b]pyridin- 2-yl)carbamoyl)pyridin- 4-yl)-2,6-diazaspiro [3.3]heptane-2-carbox- ylate | LCMS (m/z): 544 [M + H]⁺. ¹H NMR (400 MHz, DMSO-d₆) δ 13.3 (s, 1H), 9.05-9.01 (m, 1H), 8.97-8.93 (m, 1H), 8.72-8.68 (m, 2H), 8.39 (s, 1H), 7.87-7.81 (m, 2H), 6.39 (s, 1H), 4.11 (s, 4H), 4.00 (s, 4H), 2.39 (s, 3H), 1.34 (s, 9H) |
| 135 | 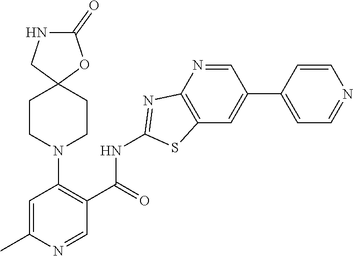 [SMILES: Cc1cc(N2CCC3(CC2)CNC(=O)O3)c(C(=O)Nc2nc3ncc(-c4ccncc4)cc3s2)cn1] | 6-methyl-4-(2-oxo-1- oxa-3,8-diazaspiro[4.5] decan-8-yl)-N-(6-(pyr- idin-4-yl)thiazolo[4,5- b]pyridin-2-yl)nicotin- amide | LCMS (m/z): 502 [M + H]⁺. ¹H NMR (400 MHz, DMSO-d₆) δ 9.09 (d, J = 2.3 Hz, 1H), 9.03 (d, J = 2.3 Hz, 1H), 8.76 (d, J = 5.3 Hz, 2H), 8.66 (s, 1H), 7.96 (d, J = 5.3 Hz, 2H), 7.58 (s, 1H), 7.39 (s, 1H), 3.55-3.46 (m, 4H), 3.31 (s, 2H), 2.53 (s, 3H), 2.00-1.87 (m, 4H) |
| 136 | 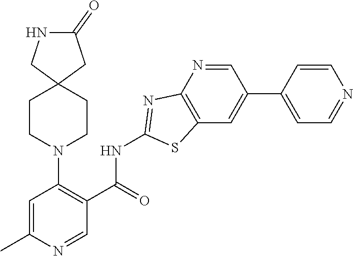 [SMILES: Cc1cc(N2CCC3(CC2)CNC(=O)C3)c(C(=O)Nc2nc3ncc(-c4ccncc4)cc3s2)cn1] | 6-Methyl-4-(3-oxo-2,8- diazaspiro[4.5]decan-8- yl)-N-(6-(pyridin-4-yl)- thiazolo[4,5-b]pyridin- 2-yl)nicotinamide | LCMS (m/z): 500 [M + H]⁺.¹H NMR (400 MHz, DMSO-d₆) δ 9.12 (d, J = 2.4 Hz, 1H), 9.06 (d, J = 2.2 Hz, 1H), 8.81 (d, J = 5.4 Hz, 2H), 8.64 (s, 1H), 8.06 (d, J = 6.0 Hz, 2H), 7.56 (s, 1H), 7.35 (s, 1H), 3.60-3.44 (m, 4H), 3.10 (s, 2H), 2.52 (s, 3H), 2.14 (s, 2H), 1.72-1.66 (m, 4H) |
| 137 | 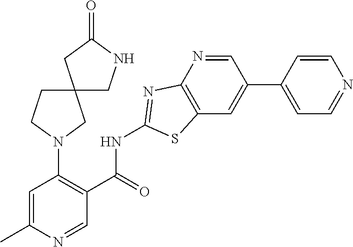 [SMILES: Cc1cc(N2CCC3(CNC(=O)C3)C2)c(C(=O)Nc2nc3ncc(-c4ccncc4)cc3s2)cn1] | (Racemic)-6-Methyl-4- (8-oxo-2,7-diazaspiro [4.4]nonan-2-yl)-N-(6- (pyridin-4-yl)thiazolo [4,5-b]pyridin-2-yl)- nicotinamide | LCMS (m/z): 486 [M + H]⁺. ¹H NMR (400 MHz, DMSO-d₆) δ 9.02 (d, J = 2.4 Hz, 1H), 8.93 (d, J = 2.2 Hz, 1H), 8.70 (d, J = 5.2 Hz, 2H), 8.36 (s, 1H), 7.88-7.81 (m, 2H), 7.62 (s, 1H), 6.67 (s, 1H), 3.43-3.34 (m, 4H), 3.21-3.14 (m, 2H), 2.41 (s, 3H), 2.20 (s, 2H), 1.97 (t, J = 7.0 Hz, 2H) |
| 138 | 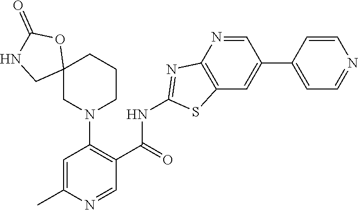 [SMILES: Cc1cc(N2CCCC3(CNC(=O)O3)C2)c(C(=O)Nc2nc3ncc(-c4ccncc4)cc3s2)cn1] | (Racemic)-6-Methyl-4- (2-oxo-1-oxa-3,7-diaza- spiro[4.5]decan-7-yl)- N-(6-(pyridin-4-yl)- thiazolo[4,5-b]pyridin- 2-yl)nicotinamide | LCMS (m/z): 502 [M + H]⁺. ¹H NMR (400 MHz, DMSO-d₆) δ 13.0 (s, 1H), 9.03 (d, J = 2.4 Hz, 1H), 8.95 (d, J = 2.4 Hz, 1H), 8.72-8.68 (m, 2H), 8.45 (s, 1H), 7.87- 7.82 (m, 2H), 7.59 (s, 1H), 7.04 (s, 1H), 3.54 (d, J = 9.2 Hz, 1H), 3.38 (d, J = 12.2 Hz, 1H), 3.23 (d, J = 9.2 Hz, 1H), 3.20-3.09 (m, 2H), 3.09- |
| | | | 2.98 (m, 1H), 2.46 (s, |
| | | | 3H), 1.90-1.71 (m, 3H), |
| | | | 1.57-1.44 (m, 1H) |
| 139 | 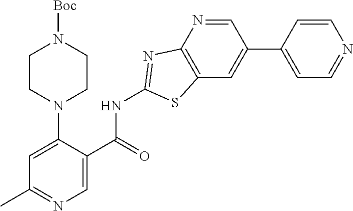 [SMILES: Cc1cc(N2CCN(C(=O)OC(C)(C)C)CC2)c(C(=O)Nc2nc3ncc(-c4ccncc4)cc3s2)cn1] | tert-Butyl 4-(2-methyl- 5-((6-(pyridin-4-yl)- thiazolo[4,5-b]pyridin- 2-yl)carbamoyl)pyridin- 4-yl)piperazine-1- carboxylate | LCMS (m/z): 532 [M + H]⁺. ¹H NMR (400 MHz, DMSO-d₆) δ 13.6 (s, 1H), 9.12 (d, J = 2.4 Hz, 1H), 9.07 (d, J = 2.4 Hz, 1H), 8.85-8.78 (m, 2H), 8.69 (s, 1H), 8.08- 8.02 (m, 2H), 7.31 (s, 1H), 3.60-3.58 (m, 4H), 2.55 (s, 3H), 1.39 (s, 9H). 4H obscured by water peak |
| 140 | 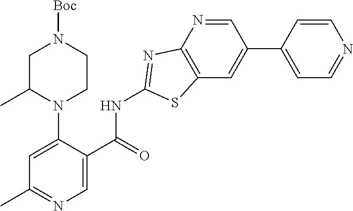 [SMILES: Cc1cc(N2CCN(C(=O)OC(C)(C)C)CC2C)c(C(=O)Nc2nc3ncc(-c4ccncc4)cc3s2)cn1] | tert-Butyl 3-methyl-4- (2-methyl-5-((6-(pyr- idin-4-yl)thiazolo[4,5- b]pyridin-2-yl)car- bamoyl)pyridin-4-yl)- piperazine-1-carbox- ylate | LCMS (m/z): 546 [M + H]⁺. ¹H NMR (400 MHz, DMSO-d₆) δ 13.3 (s, 1H), 9.03 (d, J = 2.2 Hz, 1H), 8.98 (d, J = 2.2 Hz, 1H), 8.72-8.66 (m, 2H), 8.50 (s, 1H), 7.88- 7.83 (m, 2H), 7.06 (s, 1H), 4.16 (d, J = 7.2 Hz, 1H), 3.69 (dd, J = 13.4, 4.0 Hz, 1H), 3.48 (d, J = 12.6 Hz, 1H), 3.40- 3.36 (m, 1H), 3.30-3.22 |
| | | | (m, 2H), 3.06-2.95 (m, |
| | | | 1H), 1.38 (s, 9H), 1.13 |
| | | | (d, J = 6.8 Hz, 3H) |
| 141^{&,a} | 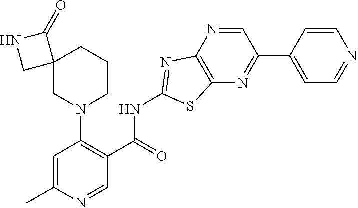 [SMILES: Cc1cc(N2CCCC3(CNC3=O)C2)c(C(=O)Nc2nc3ncc(-c4ccncc4)nc3s2)cn1] | (S or R)-6-methyl-4-(1- oxo-2,6-diazaspiro[3.5] nonan-6-yl)-N-(6-(pyr- idin-4-yl)thiazolo[4,5- b]pyrazin-2-yl)nicotin- amide | LCMS (m/z): 487 [M + H]⁺. ¹H NMR (400 MHz, DMSO-d₆) δ 13.30 (s, 1H), 8 9.33 (s, 1H), 8.74 (d, J = 5.4 Hz, 2H), 8.49 (s, 1H), 8.16 (d, J = 5.7 Hz, 2H), 7.85 (s, 1H), 7.06 (s, 1H), 3.49 (m, 1H), 3.28 (d, J = 5.3 Hz, 1H), 3.18 (m, 2H), 3.06 (d, J = 5.9 Hz, 1H), 2.92 (m, 1H), 2.46 (s, 3H), 1.71 (d, J = 7.3 Hz, 4H) |
| 142^{&,a} | 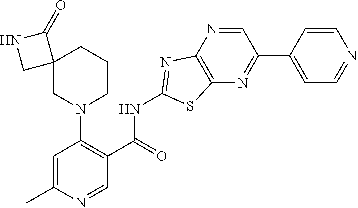 [SMILES: Cc1cc(N2CCCC3(CNC3=O)C2)c(C(=O)Nc2nc3ncc(-c4ccncc4)nc3s2)cn1] | (R or S)-6-methyl-4-(1- oxo-2,6.diazaspiro[3.5] nonan-6-yl)-N-(6-(pyr- idin-4-yl)thiazolo[4,5- b]pyrazin-2-yl)nicotin- amide | LCMS (m/z): 487 [M + H]⁺. ¹H NMR (400 MHz, DMSO-d₆) δ 13.29 (s, 1H), 9.34 (s, 1H), 8.74 (d, J = 5.3 Hz, 2H), 8.50 (s, 1H), 8.19- 8.13 (m, 2H), 7.85 (s, 1H), 7.07 (s, 1H), 3.27 (d, J = 6.0 Hz, 1H), 3.23-3.14 (m, 1H), 3.06 (d, J = 5.9 Hz, 1H), 2.92 (s, 1H), 2.52 (s, 3H), 2.46 (s, 2H), 1.63 (s, 4H) |
| 143^{&,b} | 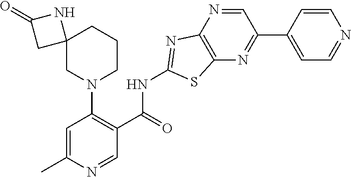 [SMILES: Cc1cc(N2CCCC3(CC(=O)N3)C2)c(C(=O)Nc2nc3ncc(-c4ccncc4)nc3s2)cn1] | (S or R)-6-methyl-4-(2- oxo-1,6-diazaspiro[3.5] nonan-6-yl)-N-(6-(pyr- idin-4-yl)thiazolo[4,5- b]pyrazin-2-yl)nicotin- amide | LCMS (m/z): 487 [M + H]⁺. ¹H NMR (300 MHz, DMSO-d₆) δ 13.30 (s, 1H), 9.35 (s, 1H), 8.75 (d, J = 5.2 Hz, 2H), 8.46 (s, 1H), 8.23 (d, J = 5.5 Hz, 3H), 7.04 (s, 1H), 3.28 (d, J = 5.3 Hz, 4H), 2.59 (m, 1H), 2.46 (s, 1H), 1.79 (d, J = 8.6 Hz, |
| | | | 3H), 1.71 (d, J = 7.3 |
| | | | Hz, 4H). |
| 144^{&,b} | 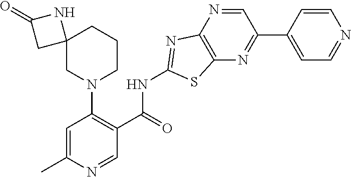 [SMILES: Cc1cc(N2CCCC3(CC(=O)N3)C2)c(C(=O)Nc2nc3ncc(-c4ccncc4)nc3s2)cn1] | (R or S)-6-methyl-4-(2- oxo-1,6-diazaspiro[3.5] nonan-6-yl)-N-(6-(pyr- idin-4-yl)thiazolo[4,5- b]pyrazin-2-yl)nicotin- amide | LCMS (m/z): 487 [M + H]⁺. ¹H NMR (300 MHz, DMSO-d₆) δ 13.28 (s, 1H), 9.34 (s, 1H), 8.78-8.70 (m, 2H), 8.46 (s, 1H), 8.22 (s, 1H), 8.20-8.11 (m, 2H), 7.02 (s, 1H), 3.17 (m, 3H), 3.03 (s, 1H), 2.77 (m, 1H), 2.64-2.51 (m, 1H), 2.45 (s, 3H), 1.67 (m, 4H) |
| 145 | 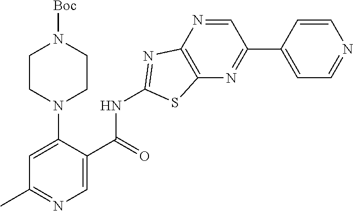 [SMILES: Cc1cc(N2CCN(C(=O)OC(C)(C)C)CC2)c(C(=O)Nc2nc3ncc(-c4ccncc4)nc3s2)cn1] | tert-butyl 4-(2-methyl- 5-((6-(pyridin-4-yl)- thiazolo[4,5-b]pyrazin- 2-yl)carbamoyl)pyridin- 4-yl)piperazine-1-car- boxylate | LCMS (m/z): 533 [M + H]⁺. ¹H NMR (300 MHz, DMSO-d₆) δ 13.38 (s, 1H), 9.34 (s, 1H), 8.75-8.73 (m, 2H), 8.50 (s, 1H), 8.17-8.13 (m, 2H), 6.97 (s, 1H), 3.45-3.33 (m, 4H), 3.17 (d, J = 5.1 Hz, 4H), 2.46 (s, 3H), 1.39 (s, 9H) |
| 146 | 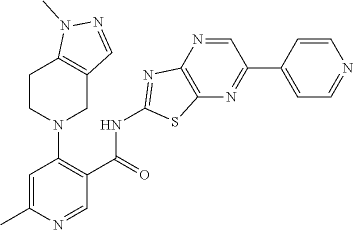 [SMILES: Cc1cc(N2CCc3c(cnn3C)C2)c(C(=O)Nc2nc3ncc(-c4ccncc4)nc3s2)cn1] | 6-methyl-4-(1-methyl- 1,4,6,7-tetrahydro-5H- pyrazolo[4,3-c]pyridin- 5-yl)-N-(6-(pyridin-4- yl)thiazolo[4,5-b]pyr- azin-2-yl)nicotinamide | LCMS (m/z): 484 [M + H]⁺. ¹H NMR (300 MHz, DMSO-d₆) δ: 13.39 (s, 1H), 9.32 (s, 1H), 8.76-8.72 (m, 2H), 8.44 (s, 1H), 8.18-8.14 (m, 2H), 7.24 (s, 1H), 7.02 (s, 1H), 4.21 (s, 2H), 3.66 (s, 3H), 3.57 (d, J = 5.9 Hz, 2H), 2.84-2.79 (m, 2H), 2.45 (s, 3H) |
| 147 | 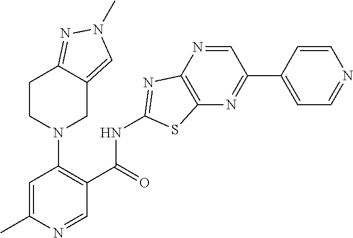 [SMILES: Cc1cc(N2CCc3nn(C)cc3C2)c(C(=O)Nc2nc3ncc(-c4ccncc4)nc3s2)cn1] | 6-methyl-4-(2-methyl- 2,4,6,7-tetrahydro-5H- pyrazolo[4,3-c]pyridin- 5-yl)-N-(6-(pyridin-4- yl)thiazolo[4,5-b]pyr- azin-2-yl)nicotinamide | LCMS (m/z): 484 [M + H]⁺. ¹H NMR (300 MHz, DMSO-d₆) δ 13.42 (s, 1H), 9.34 (s, 1H), 8.76 (d, J = 5.6 Hz, 2H), 8.44 (d, J = 2.9 Hz, 1H), 8.17-8.14 (m, 2H), 7.49 (s, 1H), 7.02 (s, 1H), 4.26-4.23 (m, 2H), 3.75 (s, 3H), 3.55 (s, 2H), 2.73 (d, J = 1.7 Hz, 2H), 2.45 (s, 3H) |
| 148 | 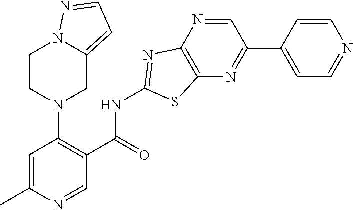 [SMILES: Cc1cc(N2CCn3nccc3C2)c(C(=O)Nc2nc3ncc(-c4ccncc4)nc3s2)cn1] | 4-(6,7-dihydropyrazolo [1,5-a]pyrazin-5(4H)- yl)-6-methyl-N-(6-(pyr- idin-4-yl)thiazolo[4,5- b]pyrazin-2-yl)nicotin- amide | LCMS (m/z): 470 [M + H]⁺. ¹H NMR (400 MHz, DMSO-d₆) δ 13.49 (s, 1H), 9.28 (s, 1H), 8.75-8.71 (m, 2H), 8.52 (s, 1H), 8.16-8.13 (m, 2H), 7.43 (d, J = 1.9 Hz, 1H), 7.02 (s, 1H), 6.15 (d, J = 1.8 Hz, 1H), 4.52 (s, 2H), 4.23- 4.19 (m, 2H), 3.68 (s, 2H), 2.47 (s, 3H) |
| 149 | 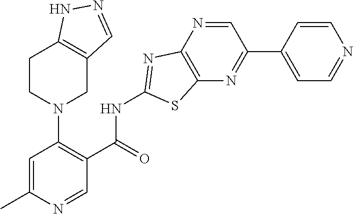 [SMILES: Cc1cc(N2CCc3[nH]ncc3C2)c(C(=O)Nc2nc3ncc(-c4ccncc4)nc3s2)cn1] | 6-methyl-N-(6-(pyridin- 4-yl)thiazolo[4,5-b]pyr- azin-2-yl)-4-(1,4,6,7- tetrahydro-5H-pyrazolo [4,3-c]pyridin-5-yl)- nicotinamide | LCMS (m/z): 470 [M + H]⁺. ¹H NMR (300 MHz, DMSO-d₆) δ 13.46 (s, 1H), 12.46 (s, 1H), 9.34 (s, 1H), 8.76- 8.74 (m, 2H), 8.44 (s, 1H), 8.17-8.15 (m, 2H), 7.45 (s, 1H), 7.04 (s, 1H), 4.27 (s, 2H), 3.57 (s, 2H), 2.78 (s, 2H), 2.45 (s, 3H) |
| 150 | 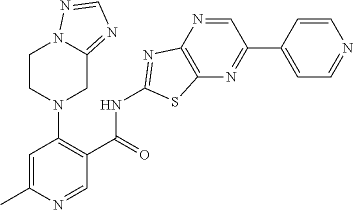 [SMILES: Cc1cc(N2CCn3ncnc3C2)c(C(=O)Nc2nc3ncc(-c4ccncc4)nc3s2)cn1] | 4-(5,6-dihydro-[1,2,4] triazolo[1,5-a]pyrazin- 7(8H)-yl)-6-methyl-N- (6-(pyridin-4-yl)thi- azolo[4,5-b]pyrazin-2- yl)nicotinamide | LCMS (m/z): 471 [M + H]⁺. ¹H NMR (400 MHz, DMSO-d₆) δ 13.50 (s, 1H), 9.38 (s, 1H), 8.83-8.68 (m, 2H), 8.55 (s, 1H), 8.23-8.13 (m, 2H), 8.00 (s, 1H), 7.18-7.10 (m, 2H), 4.58 (s, 2H), 4.25-4.22 (m, 2H), 3.76 (s, 2H), 2.67 (d, J = 1.8 Hz, 3H) |
| 151 | 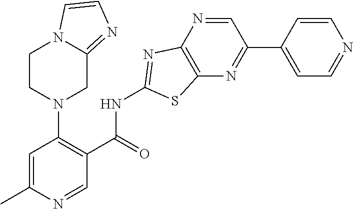 [SMILES: Cc1cc(N2CCn3ccnc3C2)c(C(=O)Nc2nc3ncc(-c4ccncc4)nc3s2)cn1] | 4-(5,6-dihydroimidazo [1,2-a]pyrazin-7(8H)- yl)-6-methyl-N-(6-(pyr- idin-4-yl)thiazolo[4,5- b]pyrazin-2-yl)nicotin- amide | LCMS (m/z): 470 [M + H]⁺. ¹H NMR (400 MHz, DMSO-d₆) δ 14.33 (s, 1H), 9.56 (s, 1H), 9.15 (s, 1H), 8.93- 8.91 (m, 2H), 8.83 (s, 1H), 8.49-8.47 (m, 2H), 7.58 (s, 1H), 7.52 (s, 1H), 4.82 (s, 2H), 4.51- 4.42 (m, 2H), 3.98 (s, 2H), 2.63 (s, 3H) |
| 152 | 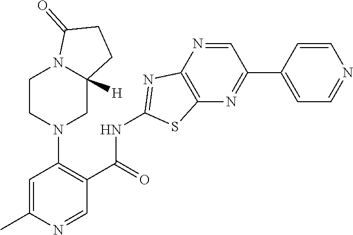 [SMILES: Cc1cc(N2CCN3C(=O)CC[C@@H]3C2)c(C(=O)Nc2nc3ncc(-c4ccncc4)nc3s2)cn1] | (R)-6-methyl-4-(6-oxo- hexahydropyrrolo[1,2- a]pyrazin-2(1H)-yl)-N- (6-(pyridin-4-yl)thi- azolo[4,5-b]pyrazin-2- yl)nicotinamide | LCMS (m/z): 487 [M + H]⁺. ¹H NMR (400 MHz, DMSO-d₆) δ 13.37 (s, 1H), 9.37 (s, 1H), 8.75 (d, J = 5.1 Hz, 2H), 8.51 (s, 1H), 8.26- 8.08 (m, 2H), 7.05 (s, 1H), 3.84 3.82 (m, 1H), 3.76 (d, J = 8.7 Hz, 1H), 3.73 (d, J = 8.7 Hz, 1H), 3.59 (s, 1H), 3.42 (s, 2H), 2.89 (t, J = 13.4 Hz, 1H), 2.73-2.69 (m, 3H), 2.47 (s, 2H), 2.13- 1.99 (m, 1H), 1.55-1.50 (m, 1H) |
| 153 | 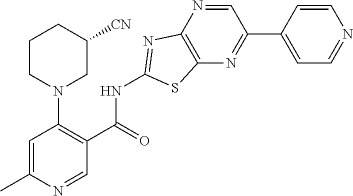 [SMILES: Cc1cc(N2CCC[C@H](C#N)C2)c(C(=O)Nc2nc3ncc(-c4ccncc4)nc3s2)cn1] | (S)-4-(3-cyanopiperi- din-1-yl)-6-methyl-N- (6-(pyridin-4-yl)thi- azolo[4,5-b]pyrazin-2- yl)nicotinamide | LCMS (m/z): 457 [M + H]⁺. ¹H NMR (300 MHz, DMSO-d₆) δ 13.36 (s, 1H), 9.26 (s, 1H), 8.73-8.71 (m, 2H), 8.47 (s, 1H), 8.15-8.13 (m, 1H), 6.98 (s, 1H), 3.59-3.51 (m, 1H), 3.28-3.25 (m, 2H), 3.15-3.13 (m, 2H), 2.43 (s, 3H), 1.80-1.77 (m, 1H), 1.74-1.57 (m, 3H) |
| 154 | 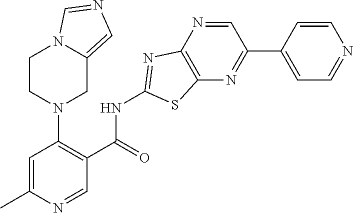 [SMILES: Cc1cc(N2CCn3cncc3C2)c(C(=O)Nc2nc3ncc(-c4ccncc4)nc3s2)cn1] | 4-(5,6-dihydroimidazo [1,5-a]pyrazin-7(8H)- yl)-6-methyl-N-(6-(pyr- idin-4-yl)thiazolo[4,5- b]pyrazin-2-yl)nicotin- amide | LCMS (m/z): 470 [M + H]⁺. ¹H NMR (400 MHz, DMSO-d₆) δ 13.51 (s, 1H), 9.29 (s, 1H), 8.81-8.64 (m, 2H), 8.51 (s, 1H), 8.18-8.12 (m, 2H), 7.13 (d, J = 1.2 Hz, 1H), 7.07 (s, 1H), 6.91 (d, J = 1.3 Hz, 1H), 4.44 (s, 2H), 4.09 (d, J = 5.3 Hz, 2H), 3.64 (d, J = 5.2 Hz, 2H), 2.47 (s, 3H) |
| 155 | 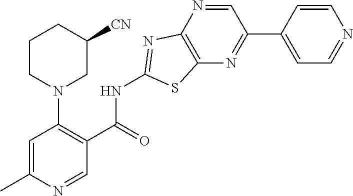 [SMILES: Cc1cc(N2CCC[C@@H](C#N)C2)c(C(=O)Nc2nc3ncc(-c4ccncc4)nc3s2)cn1] | (R)-4-(3-cyanopiperi- din-1-yl)-6-methyl-N- (6-(pyridin-4-yl)thi- azolo[4,5-b]pyrazin-2- yl)nicotinamide | LCMS (m/z): 457 [M + H]⁺. ¹H NMR (300 MHz, DMSO-d₆) δ 13.33 (s, 1H), 9.29 (s, 1H), 8.81-8.61 (m, 2H), 8.47 (s, 1H), 8.24-8.04 (m, 2H), 7.01 (s, 1H), 3.57-3.53 (m, 1H), 3.24 (s, 1H), 3.19-2.51 (m, 2H), 2.44 (s, 3H), 2.08 (s, 1H), 2.00-1.55 (m, 1H), 1.43-1.14 (m, 3H). |

^{&}Single enantiomer, unknown stereochemistry
^{a}Chiral separation conditions for Example 141 and 142: Column: CHIRAL ART Cellulose-SB, 3.0 × 50 mm; 3 μm; Mobile Phase A: (MTBE: Hex = 1:1) (0.1% DEA): EtOH = 70:30; Flow rate: 1.00 mL/min; Peak number: 2; retention time: 6.548 min (Example 141), 8.730 min (Example 142)
^{b}Chiral separation conditions of Example 143 and 144: Column: CHIRALPAK IE-3, 4.6 × 50 mm, 3 μm; Mobile Phase A: (MTBE: Hex = 1:1) (0.1% DEA): MeOH = 50:50; Flow rate: 1.67 mL/min; Peak number: 2; retention time: 1.587 min (Example 143), 2.325 min (Example 144)

### Example 156: Synthesis of 4-(4-Cyclopropyl-3-oxopiperazin-1-yl)-6-methyl-N-(6-(pyridin-4-yl)thiazolo[4,5-b]pyridin-2-yl)nicotinamide

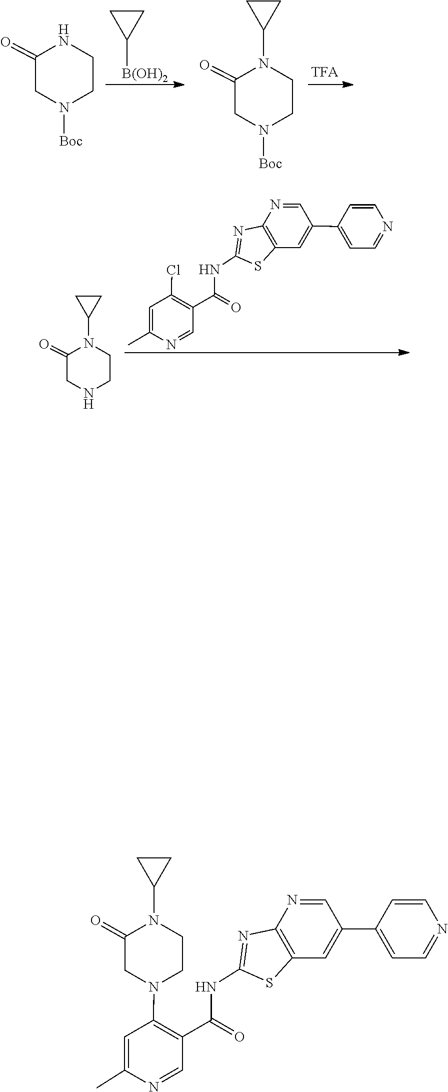

Step 1: To a solution of tert-butyl 3-oxopiperazine-1-carboxylate (1 g, 5 mmol), cyclopropylboronic acid (0.86 g, 10 mmol), Cu(OAc)₂ (0.23 g, 1.3 mmol) and DMAP (1.83 g, 15 mmol) in toluene (20 mL) was added NaHMDS (1 M in THF, 4.99 mL, 4.99 mmol) and the mixture was heated at 95° C. under an 02 balloon for 18 h. The mixture was diluted with a saturated aqueous NH₄Cl solution (50 mL), extracted with EtOAc (3×50 mL) and the combined organic layers washed with brine (30 mL), dried over Na₂SO₄, filtered and concentrated under reduced pressure. The residue was purified by silica gel chromatography (DCM/MeOH=100/1, v/v) to give tert-butyl 4-cyclopropyl-3-oxopiperazine-1-carboxylate (650 mg, 54%) as a yellow oil. LCMS (ESI, m/z):263 [M+Na]⁺, ¹H NMR (400 MHz, DMSO-d₆): δ 3.85 (s, 2H), 3.48 (t, J=5.6 Hz, 2H), 3.23 (dd, J=6.2, 4.6 Hz, 2H), 2.71 (td, J=7.2, 3.8 Hz, 1H), 1.41 (s, 9H), 0.73-0.58 (m, 4H).

Step 2: To a solution of tert-butyl 4-cyclopropyl-3-oxopiperazine-1-carboxylate (100 mg, 0.42 mmol) in DCM (2 mL) was added TFA (0.4 mL) and the mixture was stirred at room temperature for 2 h. The mixture was concentrated under reduced pressure and the residue dissolved in DMF/ACN (1 mL/1 mL). 4-Chloro-6-methyl-N-(6-(pyridin-4-yl)thiazolo[4,5-b]pyridin-2-yl)nicotinamide (79 mg, 0.21 mmol) and DIPEA (267 mg, 2.1 mmol) were added and the mixture was heated at 80° C. in a sealed-tube overnight. The mixture was diluted with water (10 mL), extracted with EtOAc (20×15 mL) and the combined organic layers washed with brine (10 mL), dried over Na₂SO₄, filtered and concentrated under reduced pressure. The residue was purified by prep-TLC (DCM/MeOH=12/1, v/v) to give 4-(4-cyclopropyl-3-oxopiperazin-1-yl)-6-methyl-N-(6-(pyridin-4-yl)thiazolo[4,5-b]pyridin-2-yl)nicotinamide (9 mg, 9%) as a yellow solid. LCMS (ESI, m/z): 486 [M+H]⁺, ¹H NMR (400 MHz, DMSO-d₆): δ 13.2 (s, 1H), 9.04 (d, J=2.3 Hz, 1H), 8.97 (d, J=2.3 Hz, 1H), 8.75-8.65 (m, 2H), 8.44 (s, 1H), 7.89-7.80 (m, 2H), 6.93 (s, 1H), 3.78 (s, 2H), 3.45-3.40, (m, 2H, partially obscured by water peak), 3.54-3.24 (m, 2H), 2.76-2.69 (m, 1H), 2.44 (s, 3H), 0.72-0.58 (m, 4H).

### Example 157: Synthesis of 4-(4-(Cyclopropylmethyl)-3-oxopiperazin-1-yl)-6-methyl-N-(6-(pyridin-4-yl)thiazolo[4,5-b]pyridin-2-yl)nicotinamide

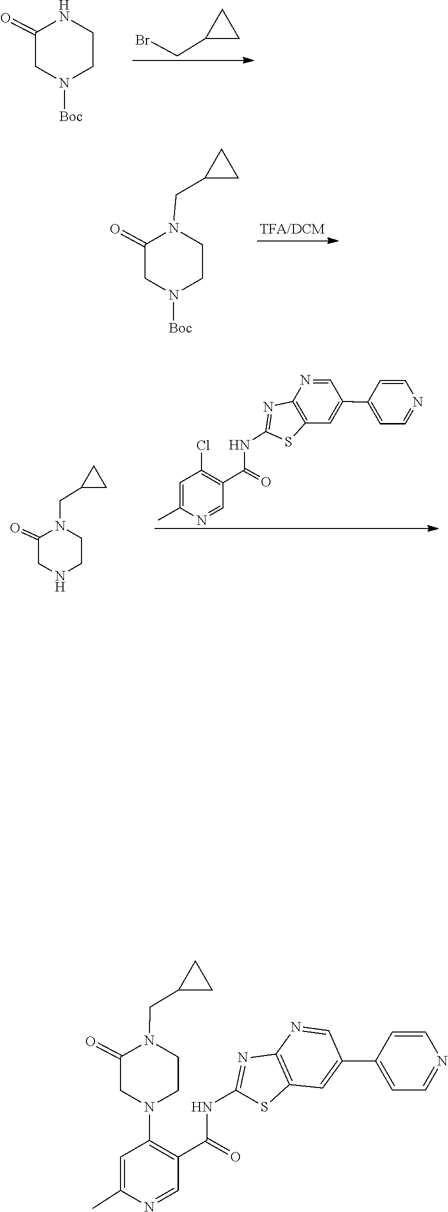 [SMILES: CC(C)(C)OC(=O)N1CCNC(=O)C1 ; BrCC1CC1 ; CC(C)(C)OC(=O)N1CCN(CC2CC2)C(=O)C1 ; O=C1CNCCN1CC1CC1 ; Cc1cc(Cl)c(C(=O)Nc2nc3ncc(-c4ccncc4)cc3s2)cn1 ; Cc1cc(N2CCN(CC3CC3)C(=O)C2)c(C(=O)Nc2nc3ncc(-c4ccncc4)cc3s2)cn1]

Step 1: To a solution of tert-butyl 3-oxopiperazine-1-carboxylate (500 mg, 2.5 mmol) in dry THF (10 mL) was added t-BuOK (420 mg, 3.75 mmol) and the mixture was stirred at room temperature for 20 min then cooled to 0° C. and (bromomethyl)cyclopropane (506 mg, 3.75 mmol) was added. The mixture was allowed to warm to room temperature and stirred for 4 h. The mixture was concentrated under reduced pressure and the residue diluted with water (30 mL), extracted with EtOAc (4×30 mL) and the combined organic layers washed with brine (20 mL), dried over Na₂SO₄, filtered and concentrated under reduced pressure to give tert-butyl 4-(cyclopropylmethyl)-3-oxopiperazine-1-carboxylate (290 mg, 46%) as a colorless oil. LCMS (ESI, m/z): 255 [2+H]⁺, ¹H NMR (400 MHz, DMSO-d₆): δ 3.88 (s, 2H), 3.54 (t, J=5.4 Hz, 2H), 3.39 (dd, J=6.4, 4.6 Hz, 2H), 3.19 (d, J=7.0 Hz, 2H), 1.40 (d, J=1.2 Hz, 9H), 0.99-0.89 (m, 1H), 0.48-0.37 (m, 2H), 0.25-0.15 (m, 2H).

Step 2: To a solution of tert-butyl 4-(cyclopropylmethyl)-3-oxopiperazine-1-carboxylate (107 mg, 0.42 mmol) in DCM (10 mL) was added TFA (2 mL) and the mixture was stirred at room temperature for 3 h. The mixture was concentrated under reduced pressure and the residue dissolved in DMF/ACN (2 mL/2 mL). 4-Chloro-6-methyl-N-(6-(pyridin-4-yl)thiazolo[4,5-b]pyridin-2-yl)nicotinamide (80 mg, 0.21 mmol) and DIPEA (270.1 mg, 2.1 mmol) were added and the mixture was heated at 80° C. in a sealed tube overnight. The mixture was diluted with water (20 mL), extracted with EtOAc (17×20 mL) and the combined organic layers washed with brine (10 mL), dried over Na₂SO₄, filtered and concentrated under reduced pressure. The residue was purified by prep-TLC (DCM/MeOH=12/1, v/v) to give 4-(4-(cyclopropylmethyl)-3-oxopiperazin-1-yl)-6-methyl-N-(6-(pyridin-4-yl)thiazolo[4,5-b]pyridin-2-yl)nicotinamide (40 mg, 38%) as a yellow solid. LCMS (ESI, m/z): 500 [M+H]⁺, ¹H NMR (400 MHz, DMSO-d₆): δ 9.03 (d, J=2.2 Hz, 1H), 8.97 (d, J=2.2 Hz, 1H), 8.73-8.67 (m, 2H), 8.47 (s, 1H), 7.88-7.82 (m, 2H), 6.99 (s, 1H), 3.84 (s, 2H), 3.60-3.55 (m, 1H), 3.50 (s, 2H), 3.37-3.32 (m, 1H, partially obscured by water peak), 3.21 (t, J=6.8 Hz, 2H), 2.46 (s, 3H), 0.99-0.89 (m, 1H), 0.49-0.42 (m, 2H), 0.26-0.22 (m, 1H), 0.19-0.17 (m, 1H).

### Example 158: Synthesis of 4-(2-Methoxyphenyl)-6-methyl-N-(6-methylimidazo[2,1-b][1,3,4]thiadiazol-2-yl)nicotinamide

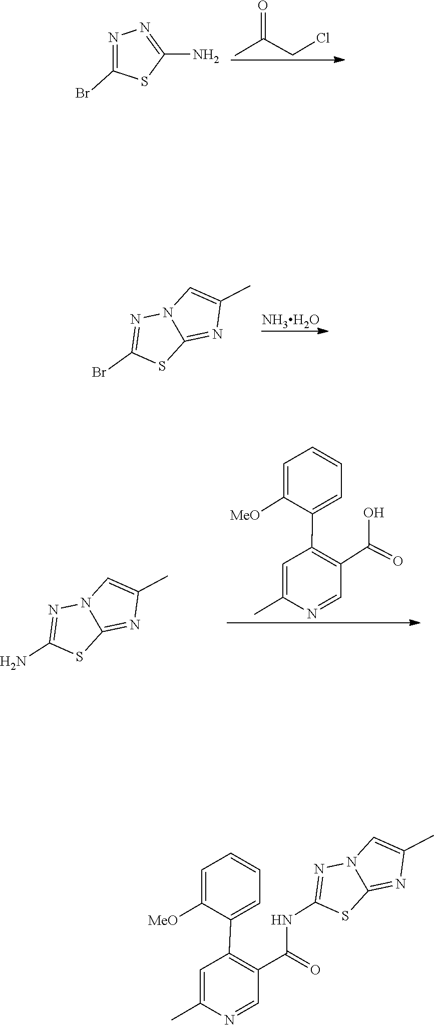

Step 1: To a solution of 5-bromo-1,3,4-thiadiazol-2-amine (1.00 g, 5.55 mmol) in water (20 mL) was added 1-chloropropan-2-one (1.54 g, 16.7 mmol) and the mixture was heated at 100° C. in a sealed tube overnight. The mixture was concentrated under reduced pressure and the residue purified by silica gel chromatography (1-2% MeOH/DCM) to give 2-bromo-6-methylimidazo[2,1-b][1,3,4]thiadiazole (770 mg, 63%) as a yellow solid. LCMS (ESI, m/z): 218, 220 [M+H], ¹H NMR (400 MHz, DMSO-d₆): δ 8.03 (s, 1H), 2.27 (s, 3H).

Step 2: To a solution of 2-bromo-6-methylimidazo[2,1-b][1,3,4]thiadiazole (200 mg, 0.92 mmol) in 1,4-dioxane (2 mL) was added concentrated aqueous NH₄OH (2 mL) and the mixture was heated at 110° C. in a sealed tube overnight. The mixture was concentrated under reduced pressure and the residue purified by prep-TLC (DCM/MeOH=15/1, v/v) to give 6-methylimidazo[2,1-b][1,3,4]thiadiazol-2-amine (79 mg, 56%) as a yellow solid. LCMS (ESI, m/z): 155.10 [M+H]⁺, ¹H NMR (400 MHz, DMSO-d₆): δ 7.40 (s, 1H), 7.34 (s, 2H), 2.16 (s, 3H).

Step 3: A solution of 6-methylimidazo[2,1-b][1,3,4]thiadiazol-2-amine (76 mg, 0.49 mmol), 4-(2-methoxyphenyl)-6-methylnicotinic acid (100 mg, 0.41 mmol), EDCI·HCl (118 mg, 0.61 mmol) and DMAP (75 mg, 0.61 mmol) in DCM (10 mL) was stirred at room temperature overnight. The mixture was diluted with water (10 mL), extracted with DCM (30 mL×3) and the combined organic layers washed with brine (20 mL), dried over Na₂SO₄, filtered and concentrated under reduced pressure. The residue was purified by prep-TLC (DCM/MeOH=15/1, v/v) to give 4-(2-methoxyphenyl)-6-methyl-N-(6-methylimidazo[2,1-b][1,3,4]thiadiazol-2-yl)nicotinamide (90 mg, 58%) as a yellow solid. LCMS (ESI, m/z): 380 [M+H]⁺, ¹H NMR (400 MHz, DMSO-d₆): δ 12.9 (s, 1H), 8.69 (s, 1H), 7.68 (s, 1H), 7.43-7.36 (m, 2H), 7.33 (s, 1H), 7.08 (t, J=7.2 Hz, 1H), 6.99 (d, J=8.2 Hz, 1H), 3.54 (s, 3H), 2.58 (s, 3H), 2.23 (s, 3H).

TABLE T
The following examples were prepared using a similar procedure to that described for Example 158

| Ex. | | | |
|---|---|---|---|
| No. | Structure | Compound Name | Characterization Data |
| 159 | 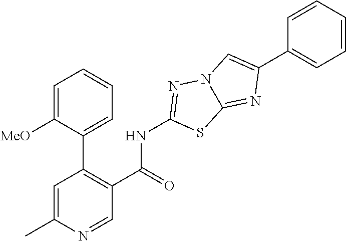 [SMILES: COc1ccccc1-c1cc(C)ncc1C(=O)Nc1nn2cc(-c3ccccc3)nc2s1] | 4-(2-methoxyphenyl)- 6-methyl-N-(6-phen- ylimidazo[2,1-b] [1,3,4]thiadiazol-2- yl)nicotinamide | LCMS (m/z): 442 [M + H]⁺. ¹H NMR (400 MHz, DMSO- d₆) δ 13.1 (s, 1H), 8.72 (s, 1H), 8.52 (s, 1H), 7.85 (d, J = 7.2 Hz, 2H), 7.45-7.36 (m, 4H), 7.35 (s, 1H), 7.25 (t, J = 7.4 Hz, 1H), 7.10 (t, J = 7.4 Hz, 1H), 7.01 (d, J = 8.2 Hz, 1H), 3.56 (s, 3H), 2.59 (s, 3H) |

### Example 160: Synthesis of N-(6-Bromothiazolo[4,5-b]pyridin-2-yl)-6-methyl-4-(4-(thiazol-2-yl)piperazin-1-yl)nicotinamide

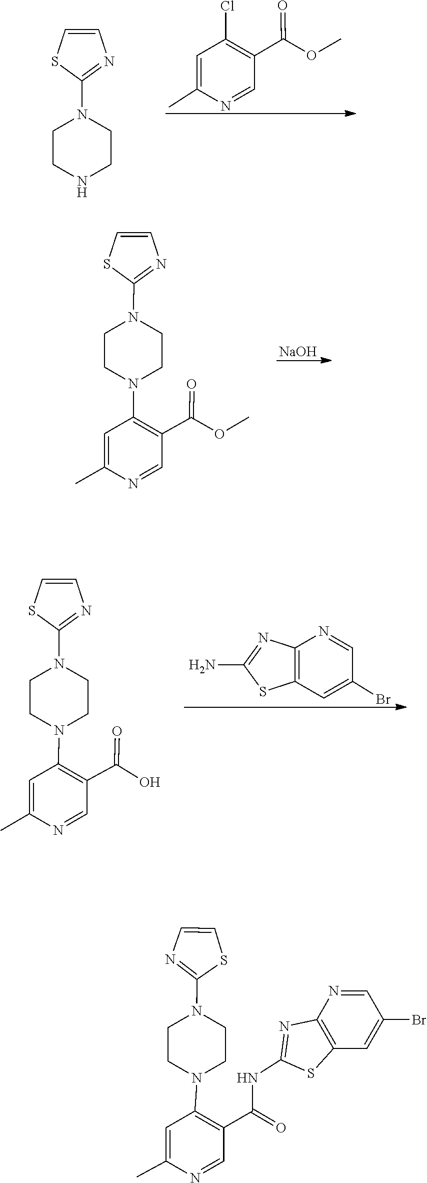

Step 1: A mixture of 2-(piperazin-1-yl)thiazole (500 mg, 2.96 mmol), methyl 4-chloro-6-methylnicotinate (275 mg, 1.48 mmol) and DIPEA (383 mg, 2.96 mmol) in ACN (5 mL) was heated at 80° C. in a sealed tube for 16 h. After cooling to room temperature, the mixture was concentrated under reduced pressure and the residue purified by silica gel chromatography (Pet. ether/EtOAc=10/1-3/1 to DCM/MeOH=50/1-20/1, v/v) to give methyl 6-methyl-4-(4-(thiazol-2-yl)piperazin-1-yl)nicotinate (340 mg, 72%) as alight yellow solid. LCMS (ESI, m/z): 319 [M+H]⁺, ¹H NMR (400 MHz, DMSO-d₆): δ 8.50 (s, 1H), 7.22-7.17 (m, 1H), 6.92-6.86 (m, 2H), 3.83 (s, 3H), 3.55-3.50 (m, 4H), 3.30-3.27 (m, 4H), 2.41 (s, 3H).

Step 2: To a solution of methyl 6-methyl-4-(4-(thiazol-2-yl)piperazin-1-yl)nicotinate (425 mg, 1.33 mmol) in MeOH (9 mL) and water (9 mL) was added NaOH (80 mg, 2.0 mmol) and the mixture was heated at 50° C. for 2 h. After cooling to room temperature, the mixture was concentrated under reduced pressure to remove most of the MeOH. The aqueous residue was adjusted to pH 4-5 with 6 M aqueous HCl then concentrated under reduced pressure. The residue was purified by prep-HPLC to give 6-methyl-4-(4-(thiazol-2-yl)piperazin-1-yl)nicotinic acid (236 mg, 58%) as a white solid. LCMS (ESI, m/z): 305 [M+H]⁺, ¹H NMR (400 MHz, DMSO-d₆): δ 8.50 (s, 1H), 7.20 (d, J=3.4 Hz, 1H), 6.88 (d, J=3.8 Hz, 2H), 3.56-3.49 (m, 4H), 3.33 (s, 4H), 2.40 (s, 3H).

Step 3: To a solution of 6-methyl-4-(4-(thiazol-2-yl)piperazin-1-yl)nicotinic acid (50 mg, 0.16 mmol) in DMF (4 mL) was added 6-bromothiazolo[4,5-b]pyridin-2-amine (38 mg, 0.16 mmol), HATU (94 mg, 0.25 mmol) and DIPEA (106 mg, 0.82 mmol) and the mixture was heated at 80° C. for 16 h. After cooling to room temperature, the mixture was diluted with water (5 mL), extracted with EtOAc (3×15 mL) and the combined organic phases dried over Na₂SO₄, filtered and concentrated under reduced pressure. The residue was purified by prep-TLC (DCM/MeOH=10/1, v/v) and prep-HPLC to give N-(6-bromothiazolo[4,5-b]pyridin-2-yl)-6-methyl-4-(4-(thiazol-2-yl)piperazin-1-yl)nicotinamide (4 mg, 5%) as a white solid. LCMS (ESI, m/z): 516, 518 [M+H]⁺, ¹H NMR (400 MHz, DMSO-d₆): δ 8.82-8.74 (m, 1H), 8.68-8.61 (m, 1H), 8.48 (s, 1H), 7.17 (d, J=3.6 Hz, 1H), 7.01 (s, 1H), 6.87 (d, J=3.6 Hz, 1H), 3.55-3.49 (m, 4H), 3.29 (s, 4H), 2.46 (s, 3H).

TABLE U
The following examples were prepared using a similar procedure to that described for Example 160

| Ex. | | | |
|---|---|---|---|
| No. | Structure | Compound Name | Characterization Data |
| 161 | 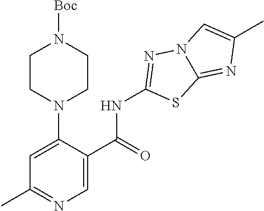 | tert-Butyl 4-(2-methyl- 5-((6-methylimidazo [2,1-b][1,3,4]thiadiazol- 2-yl)carbamoyl)pyr- idin-4-yl)piperazine-1- carboxylate | LCMS (m/z): 458 [M + H]⁺. ¹H NMR (400 MHz, DMSO- d₆) δ 8.39 (s, 1H), 8.13 (s, 1H), 7.66 (s, 1H), 6.93 (s, 1H), 3.45-3.40 (m, 4H), 3.17-3.13 (m, 4H), 2.43 (s, 3H), 2.23 (s, 3H), 1.39 (s, 9H) |

### Example 162: Synthesis of N-(6-(1-cyanocyclopropyl)thiazolo[4,5-b]pyrazin-2-yl)-4-(2-methoxyphenyl)-6-methylnicotinamide

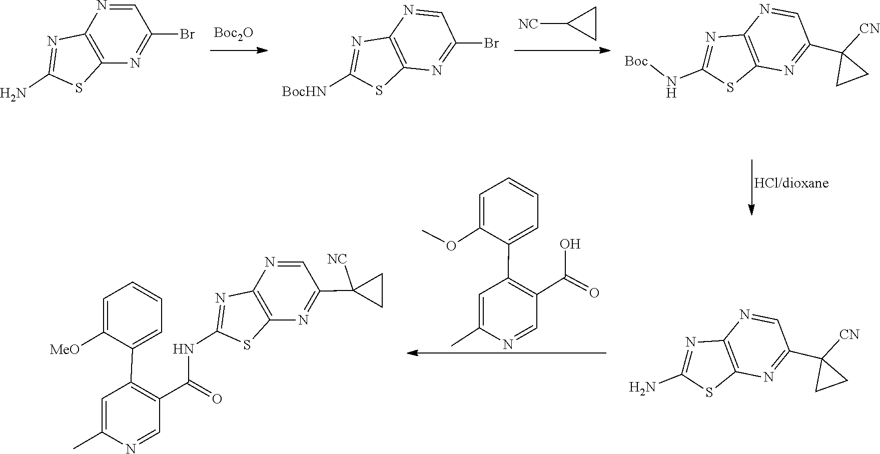

Step 1: To a solution of 6-bromothiazolo[4,5-b]pyrazin-2-amine (1 g, 4.35 mmol) in DMF (10 mL) was added Boc₂O (1.9 g, 8.7 mmol), TEA (1.32 g, 13.1 mmol) and DMAP (106 mg, 0.87 mmol) and the mixture was stirred at room temperature overnight. The mixture was diluted with water (50 mL), extracted with EtOAc (2×50 mL) and the combined organic layers washed with water (50 mL), brine (50 mL), dried over Na₂SO₄, filtered and concentrated under reduced pressure. The residue was purified by silica gel chromatography (Pet. ether/EtOAc=50/1-10/1, v/v) to afford tert-butyl (6-bromothiazolo[4,5-b]pyrazin-2-yl)carbamate (1.1 g, 79%) as a white solid. LCMS (ESI, m/z): 353 [M+Na]⁺, ¹H NMR (400 MHz, DMSO-d₆): δ 8.70 (s, 1H), 1.53 (s, 9H).

Step 2: To a solution of tert-butyl (6-bromothiazolo[4,5-b]pyrazin-2-yl)carbamate (200 mg, 0.606 mmol), cyclopropanecarbonitrile (122 mg, 1.82 mmol), BINAP (75.5 mg, 0.121 mmol) and Pd₂(dba)₃ (55.5 mg, 0.061 mmol) in THF (4 mL) was added NaHMDS (1 M in THF, 1.5 mL, 1.5 mmol) and the mixture was heated at 100° C. overnight. The reaction was quenched with a saturated aqueous NH₄Cl solution, and the mixture was diluted with water (30 mL) and extracted with EtOAc (2×30 mL). The combined organic layers were washed with brine (30 mL), dried over Na₂SO₄, filtered and concentrated under reduced pressure. The residue was purified by prep-TLC (Pet. ether/EtOAc=3/1, v/v) to afford tert-butyl (6-(1-cyanocyclopropyl)thiazolo[4,5-b]pyrazin-2-yl)carbamate (10 mg, 5%) as a yellow solid. LCMS (ESI, m/z): 318.15 [M+H]⁺.

Step 3: A mixture of tert-butyl (6-(1-cyanocyclopropyl)thiazolo[4,5-b]pyrazin-2-yl)carbamate (10 mg, 0.032 mmol) and 4 M HCl/dioxane (5 mL) was stirred at room temperature overnight. The mixture was concentrated under reduced pressure and the residue dissolved in DMF (2 mL). 4-(2-Methoxyphenyl)-6-methylnicotinic acid (16.8 mg, 0.069 mmol), EDCI·HCl (13.3 mg, 0.069 mmol) and DMAP (8.4 mg, 0.069 mmol) were added, and the mixture was stirred at room temperature overnight. The mixture was diluted with water (10 mL), extracted with EtOAc (2×10 mL) and the combined organic layers washed with brine (10 mL), dried over Na₂SO₄, filtered and concentrated under reduced pressure. The residue was purified by prep-HPLC to afford N-(6-(1-cyanocyclopropyl)thiazolo[4,5-b]pyrazin-2-yl)-4-(2-methoxyphenyl)-6-methylnicotinamide (1.1 mg, 5%) as a yellow solid. LCMS (ESI, m/z): 443 [M+H]⁺, ¹H NMR (400 MHz, DMSO-d₆): δ 13.3 (s, 1H), 8.80 (s, 1H), 8.60 (s, 1H), 7.41-7.31 (m, 2H), 7.26 (s, 1H), 7.09-7.03 (m, 1H), 6.98-6.94 (m, 1H), 3.50 (s, 3H), 2.56 (s, 3H), 1.89-1.83 (m, 2H), 1.79-1.72 (m, 2H).

### Example 163: Synthesis of (S)—N-(6-(5-(1-Cyanocyclopropyl)pyridin-2-yl)thiazolo[4,5-b]pyridin-2-yl)-6-methyl-4-(3-oxotetrahydro-3H-oxazolo[3,4-a]pyrazin-7(1H)-yl)nicotinamide

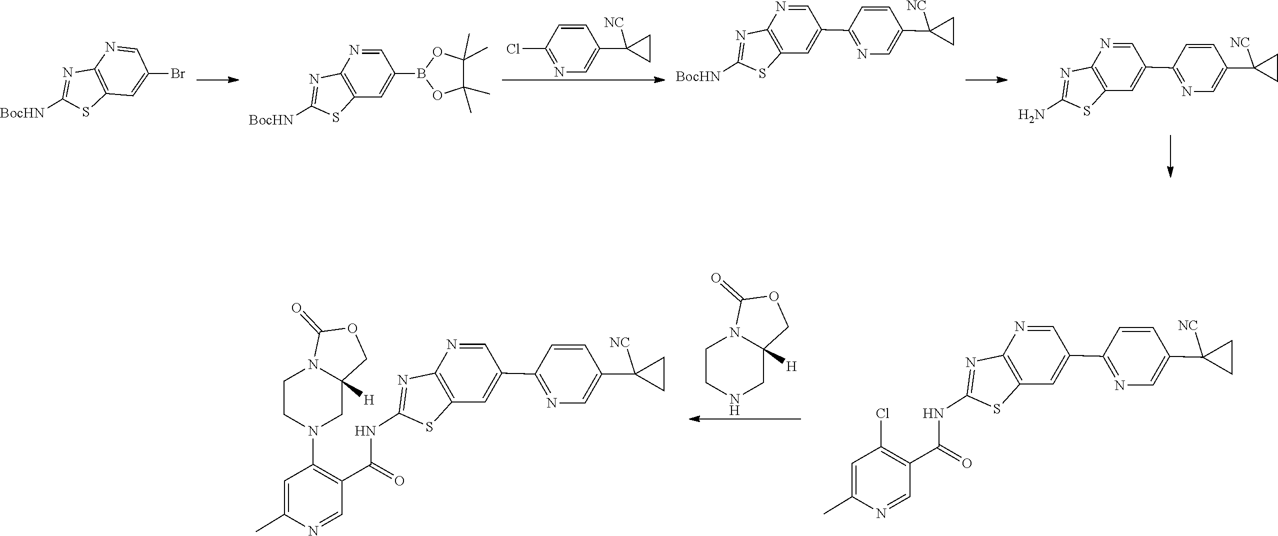

Step 1: A solution of tert-butyl (6-bromothiazolo[4,5-b]pyridin-2-yl)carbamate (4.5 g, 13.6 mmol), Pin₂B₂ (6.92 g, 27.3 mmol), Pd(dppf)Cl₂ (1.48 g, 2.04 mmol) and KOAc (6.69 g, 68.1 mmol) in dioxane (60 mL) was heated at 100° C. overnight under N2. After concentration, the residue was dissolved in Pet. ether/EtOAc (3/1, 100 mL), filtered and the filtrate concentrated under reduced pressure to afford tert-butyl (6-(4,4,5,5-tetramethyl-1,3,2-dioxaborolan-2-yl)thiazolo[4,5-b]pyridin-2-yl)carbamate (8.8 g) as a brown solid, which was used directly in the next step. LCMS (ESI, m/z):296.15 [M+H]⁺.

Step 2: A mixture of tert-butyl (6-(4,4,5,5-tetramethyl-1,3,2-dioxaborolan-2-yl)thiazolo[4,5-b]pyridin-2-yl)carbamate (8.8 g), 1-(6-chloropyridin-3-yl)cyclopropane-1-carbonitrile (1.5 g, 8.4 mmol), Pd(PPh₃)₄ (970 mg, 0.839 mmol) and Na₂CO₃ (2.67 g, 25.2 mmol) in 1,4-dioxane (60 mL) and water (15 mL) was heated at reflux overnight. The mixture was concentrated under reduced pressure and the residue purified by silica gel chromatography (Gradient: 0-2% MeOH/DCM) to afford tert-butyl (6-(5-(1-cyanocyclopropyl)pyridin-2-yl)thiazolo[4,5-b]pyridin-2-yl)carbamate (2.2 g, 66%) as a brown solid. LCMS (ESI, m/z):394.15 [M+H]⁺.

Step 3: A mixture of tert-butyl (6-(5-(1-cyanocyclopropyl)pyridin-2-yl)thiazolo[4,5-b]pyridin-2-yl)carbamate (2.2 g, 5.60 mmol) and 4 M HCl/dioxane (30 mL) was stirred at RT for 2 h. The mixture was concentrated under reduced pressure and the residue suspended in DCM (50 mL), filtered and the filter cake dried to afford 1-(6-(2-aminothiazolo[4,5-b]pyridin-6-yl)pyridin-3-yl)cyclopropane-1-carbonitrile (650 mg, 40%) as a brown solid. LCMS (ESI, m/z):294.05 [M+H]⁺.

Step 4: A solution of 1-(6-(2-aminothiazolo[4,5-b]pyridin-6-yl)pyridin-3-yl)cyclopropane-1-carbonitrile (600 mg, 2.05 mmol), 4-chloro-6-methylnicotinic acid (701 mg, 4.09 mmol), TCFH (1.72 g, 6.14 mmol) and NMI (1.68 g, 20.5 mmol) in NMP (15 mL) was stirred at room temperature overnight. The mixture was poured into water (50 mL) and the resulting precipitate collected by filtration and dried to afford 4-chloro-N-(6-(5-(1-cyanocyclopropyl)pyridin-2-yl)thiazolo[4,5-b]pyridin-2-yl)-6-methylnicotinamide (650 mg, 71%) as a brown solid. LCMS (ESI, m/z): 447.00 [M+H]⁺.

Step 5: A solution of 4-chloro-N-(6-(5-(1-cyanocyclopropyl)pyridin-2-yl)thiazolo[4,5-b]pyridin-2-yl)-6-methylnicotinamide (50 mg, 0.111 mmol), (S)-hexahydro-3H-oxazolo[3,4-a]pyrazin-3-one (80 mg, 0.447 mmol) and DIPEA (145 mg, 1.12 mmol) in NMP (1.5 mL) was heated at 80° C. overnight. The mixture was poured into water (20 mL), extracted with EtOAc (2×30 mL) and the combined organic layers washed with brine (20 mL), dried over Na₂SO₄, filtered and concentrated under reduced pressure. The residue was purified by prep-TLC (DCM/MeOH=20/1, v/v) to give (S)—N-(6-(5-(1-cyanocyclopropyl)pyridin-2-yl)thiazolo[4,5-b]pyridin-2-yl)-6-methyl-4-(3-oxotetrahydro-3H-oxazolo[3,4-a]pyrazin-7(1H)-yl)nicotinamide (40 mg, 60%) as a yellow solid. LCMS (ESI, m/z): 553.10 [M+H]⁺, ¹H NMR (400 MHz, DMSO-d₆): δ 12.9 (br s, 1H), 9.28 (d, J=2.2 Hz, 1H), 9.13 (d, J=2.2 Hz, 1H), 8.73 (d, J=2.5 Hz, 1H), 8.54 (s, 1H), 8.11 (d, J=8.4 Hz, 1H), 7.89 (dd, J=8.4, 2.5 Hz, 1H), 7.04 (s, 1H), 4.38 (t, J=8.2 Hz, 1H), 4.07-3.94 (m, 2H), 3.68-3.56 (m, 2H), 3.44-3.37 (m, 1H), 3.15-3.13 (m, 1H, partially obscured by water peak), 3.00-2.88 (m, 2H), 2.48 (s, 3H), 1.87-1.83 (m, 2H), 1.70-1.65 (m, 2H).

TABLE V
The following examples were prepared using a similar procedure to that described for Example 163

| Ex. | | | |
|---|---|---|---|
| No. | Structure | Compound Name | Characterization Data |
| 164 | 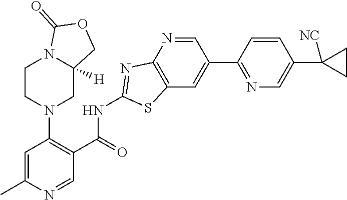 [SMILES: Cc1cc(N2CCN3C(=O)OC[C@H]3C2)c(C(=O)Nc2nc3ncc(-c4ccc(C5(C#N)CC5)cn4)cc3s2)cn1] | (R)-N-(6-(5-(1-Cyano- cyclopropyl)pyridin-2- yl)thiazolo[4,5-b]pyr- idin-2-yl)-6-methyl-4- (3-oxotetrahydro-3H- oxazolo[3,4-a]pyrazin- 7(1H)-yl)nicotinamide | LCMS (m/z): 553 [M + H]⁺. ¹H NMR (400 MHz, DMSO- d₆) δ 13.0 (s, 1H), 9.28 (d, J = 2.2 Hz, 1H), 9.13 (d, J = 2.2 Hz, 1H), 8.73 (d, J = 2.5 Hz, 1H), 8.54 (s, 1H), 8.11 (d, J = 8.4 Hz, 1H), 7.89 (dd, J = 8.4, 2.5 Hz, 1H), 7.04 (s, 1H), 4.38 (t, J = 8.2 Hz, 1H), 4.07- 3.95 (m, 2H), 3.68-3.56 (m, 2H), 3.44-3.36 (m, 1H), 3.15- 3.12 (m, 1H), 2.99-2.88 (m, 2H), 2.48 (s, 3H), 1.87-1.82 |
| | | | (m, 2H), 1.70-1.65 (m, 2H) |

### Example 165: Synthesis of N-(6-(4-Chlorophenyl)thiazolo[4,5-b]pyridin-2-yl)-5-(2-methoxyphenyl)pyridazine-4-carboxamide

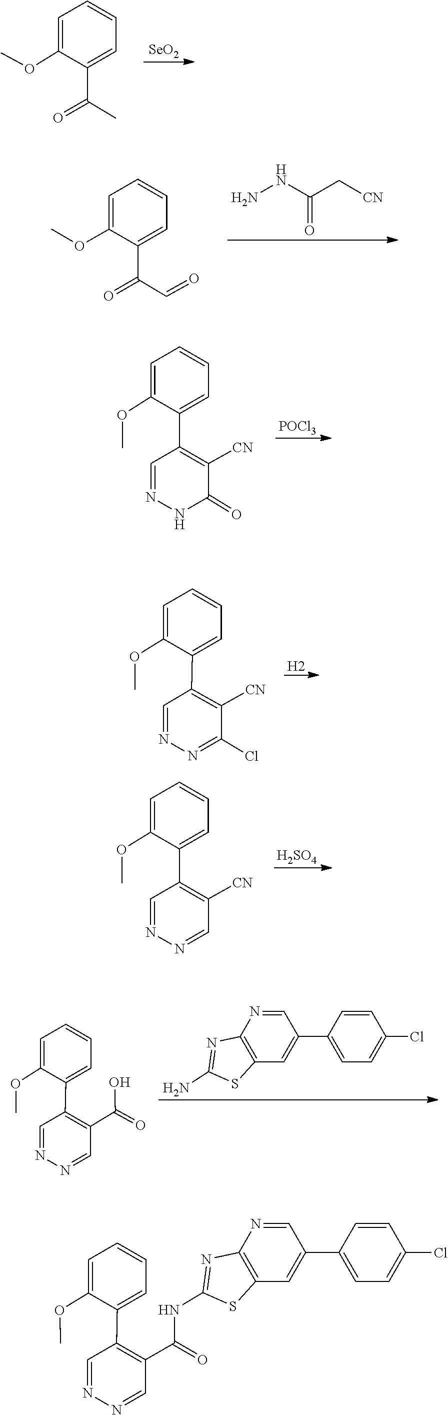

Step 1: To a solution of 1-(2-methoxyphenyl)ethan-1-one (200 mg, 1.33 mmol) in 1,4-dioxane/water (30/1, 5 mL) was added SeO₂ (296 mg, 2.66 mmol) and the mixture was heated at 60° C. overnight. The mixture was filtered and the filter cake washed with acetone (100 mL). The combined filtrates were concentrated under reduced pressure and the residue purified by silica gel chromatography (Pet. Ether/EtOAc=5/1) to give 2-(2-methoxyphenyl)-2-oxoacetaldehyde (140 mg, 31%) as a black oil. ¹H NMR (400 MHz, DMSO-d₆): δ 9.73 (s, 1H), 7.55-7.50 (m, 2H), 7.28 (dd, J=8.8, 0.8 Hz, 1H), 7.04-7.00 (m, 1H), 3.88 (s, 3H).

Step 2: To a solution of 2-(2-methoxyphenyl)-2-oxoacetaldehyde (1.28 g, 7.8 mmol) in EtOH (80 mL) was added 2-cyanoacetohydrazide (0.77 g, 7.8 mmol) and Na₂SO₄ (2.07 mg, 14.6 mmol) and the mixture was stirred at 25° C. for 5 h. The mixture was filtered, the filter cake was washed with EtOAc (3×100 mL) and the combined filtrates concentrated under reduced pressure. The residue was dissolved in EtOH (60 mL), cooled to 0° C., NaOEt (0.531 g, 7.8 mmol) was added and the mixture heated at 80° C. overnight. The mixture was diluted with water (40 mL), extracted with EtOAc (3×60 mL) and the combined organic phases dried over Na₂SO₄, filtered and concentrated under reduced pressure. The residue was purified by silica gel chromatography (2.9% MeOH/DCM) to give 5-(2-methoxyphenyl)-3-oxo-2,3-dihydropyridazine-4-carbonitrile (794 mg, 45%) as a yellow solid. LCMS (ESI, m/z): 228 [M+H]⁺, ¹H NMR (400 MHz, DMSO-d₆): δ 13.9 (s, 1H), 8.12 (s, 1H), 7.62-7.53 (m, 1H), 7.50 (dd, J=7.8, 1.8 Hz, 1H), 7.25 (d, J=8.4 Hz, 1H), 7.14 (td, J=7.6, 1.0 Hz, 1H), 3.84 (s, 3H).

Step 3: A solution of 5-(2-methoxyphenyl)-3-oxo-2,3-dihydropyridazine-4-carbonitrile (32 mg, 0.141 mmol) in POCl₃ (1 mL) was heated at 100° C. overnight. The mixture was adjusted to pH 7 with aqueous NaOH, then diluted with water (10 mL) and extracted with EtOAc (3×15 mL). The combined organic phases were dried over Na₂SO₄, filtered and concentrated under reduced pressure. The residue was purified by prep-TLC (Pet. Ether/EtOAc=3/1, v/v) to give 3-chloro-5-(2-methoxyphenyl)pyridazine-4-carbonitrile (24 mg, 69%) as a yellow solid. LCMS (ESI, m/z): 246 [M+H], ¹H NMR (400 MHz, DMSO-d₆): δ 9.60 (s, 1H), 7.66-7.60 (m, 1H), 7.57 (dd, J=7.8, 1.8 Hz, 1H), 7.30 (d, J=8.4 Hz, 1H), 7.20 (d, J=1.0 Hz, 1H), 3.85 (s, 3H).

Step 4: To a solution of 3-chloro-5-(2-methoxyphenyl)pyridazine-4-carbonitrile (24 mg, 0.098 mmol) in THF (3 mL) was added TEA (30 mg, 0.3 mmol) and 10% Pd/C (2.4 mg) and the mixture was stirred under a H₂ atmosphere (1 atm) at 25° C. overnight. The mixture was filtered and the filtrate concentrated under reduced pressure. The residue was purified by prep-TLC (Pet. Ether/EtOAc=5/1, v/v) to give 5-(2-methoxyphenyl)pyridazine-4-carbonitrile (13 mg, 63%) as a yellow solid. LCMS (ESI, m/z): 212 [M+H]⁺, ¹H NMR (400 MHz, DMSO-d₆): δ 9.68 (d, J=1.2 Hz, 1H), 9.58 (d, J=1.2 Hz, 1H), 7.65-7.57 (m, 1H), 7.53 (dd, J=7.6, 1.8 Hz, 1H), 7.29 (d, J=8.4 Hz, 1H), 7.18 (dd, J=8.0, 7.0 Hz, 1H), 3.83 (s, 3H).

Step 5: To a solution of 5-(2-methoxyphenyl)pyridazine-4-carbonitrile (85 mg, 0.402 mmol) in water (3 mL) was added conc. H₂SO₄ (10.6 mL) and the mixture was heated at 100° C. for 5 h. The mixture was adjusted to pH 5 with a saturated aqueous NaHCO₃ solution and concentrated under reduced pressure. The residue was dissolved in DCM (100 mL), filtered and the filtrate concentrated under reduced pressure. The residue was purified by prep-TLC (DCM/MeOH=20/1, v/v) to give 5-(2-methoxyphenyl)pyridazine-4-carboxylic acid (85 mg, 73%) as a white solid. LCMS (ESI, m/z): 231 [M+H]⁺, ¹H NMR (400 MHz, DMSO-d₆): δ 8.98 (d, J=1.2 Hz, 1H), 8.92 (d, J=1.2 Hz, 1H), 7.40-7.31 (m, 2H), 7.08 (d, J=8.2 Hz, 1H), 7.00-6.94 (m, 1H), 3.73 (s, 3H).

Step 6: To a solution of 5-(2-methoxyphenyl)pyridazine-4-carboxylic acid (20 mg, 0.869 mmol) in NMP (1 mL) was added HATU (39.7 mg, 0.104 mmol) and DIPEA (33.7 mg, 0.261 mmol) and the mixture was stirred at 25° C. for 2 h. 6-(4-Chlorophenyl)thiazolo[4,5-b]pyridin-2-amine (22.7 mg, 0.869 mmol) was added and the mixture was heated at 50° C. overnight. The mixture was diluted with water (10 mL), extracted with EtOAc (3×20 mL) and the combined organic phases washed with water (20 mL), brine (20 mL), dried over Na₂SO₄, filtered and concentrated under reduced pressure. The residue was purified by prep-HPLC to give N-(6-(4-chlorophenyl)thiazolo[4,5-b]pyridin-2-yl)-5-(2-methoxyphenyl)pyridazine-4-carboxamide (1.88 mg, 4%) as a yellow solid. LCMS (ESI, m/z): 474 [M+H]⁺, ¹H NMR (400 MHz, DMSO-d₆): δ 9.50 (d, J=1.0 Hz, 1H), 9.42 (s, 1H), 8.88 (s, 1H), 8.77 (s, 1H), 7.81 (d, J=8.6 Hz, 2H), 7.58 (d, J=8.4 Hz, 2H), 7.54 (d, J=7.8 Hz, 1H), 7.48 (t, J=7.8 Hz, 1H), 7.15 (t, J=7.6 Hz, 1H), 7.06 (d, J=8.4 Hz, 1H), 3.54 (s, 3H).

### Example 166: Synthesis of N-(6-(1-Cyanocyclopropyl)imidazo[2,1-b][1,3,4]thiadiazol-2-yl)-4-(2-methoxyphenyl)-6-methylnicotinamide

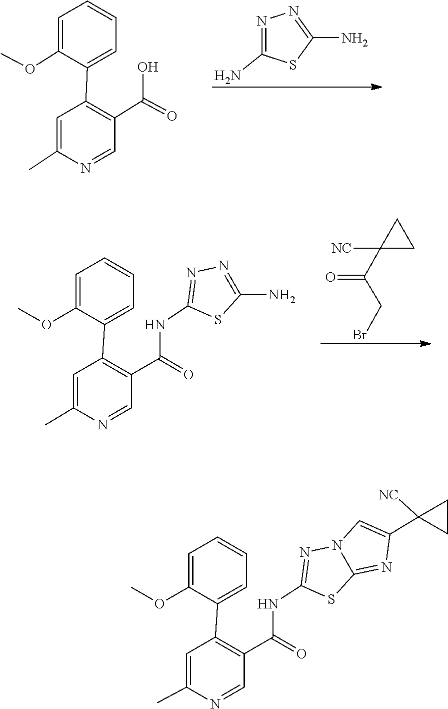 [SMILES: COc1ccccc1-c1cc(C)ncc1C(=O)O ; Nc1nnc(N)s1 ; COc1ccccc1-c1cc(C)ncc1C(=O)Nc1nnc(N)s1 ; N#CC1(C(=O)CBr)CC1 ; COc1ccccc1-c1cc(C)ncc1C(=O)Nc1nn2cc(C3(C#N)CC3)nc2s1]

Step 1: To a solution of 4-(2-methoxyphenyl)-6-methylnicotinic acid (500 mg, 2.06 mmol) in NMP (10 mL) was added HATU (1.17 g, 3.08 mmol) and DIPEA (797 mg, 6.17 mmol) and the mixture was stirred at room temperature for 1 h. 1,3,4-Thiadiazole-2,5-diamine (263 mg, 2.26 mmol) was added and the mixture was heated at 80° C. overnight. The mixture was diluted with water (30 mL), extracted with EtOAc (50 mL×3) and the combined organic layers washed with brine (20 mL), dried over Na₂SO₄, filtered and concentrated under reduced pressure. The residue was purified by silica gel chromatography (DCM/MeOH=100/1 to 10/1, v/v) to afford N-(5-amino-1,3,4-thiadiazol-2-yl)-4-(2-methoxyphenyl)-6-methylnicotinamide (521 mg, 74%) as a yellow solid. LCMS (ESI, m/z): 342 [M+H]⁺, ¹H NMR (400 MHz, DMSO-d₆): δ 12.2 (s, 1H), 8.62 (s, 1H), 7.38 (td, J=8.0, 7.6, 1.6 Hz, 1H), 7.32 (dd, J=7.6, 1.6 Hz, 1H), 7.27 (s, 1H), 7.06 (t, J=7.4 Hz, 1H), 6.98 (d, J=8.4 Hz, 1H), 6.71 (s, 2H), 3.53 (s, 3H), 2.55 (s, 3H).

Step 2: To a solution of N-(5-amino-1,3,4-thiadiazol-2-yl)-4-(2-methoxyphenyl)-6-methylnicotinamide (100 mg, 0.531 mmol) in EtOH (3 mL) was added 1-(2-bromoacetyl)cyclopropane-1-carbonitrile (182 mg, 0.531 mmol) and the mixture was stirred at room temperature for 4 h then heated at 100° C. overnight. The mixture was diluted with water (20 mL), extracted with EtOAc (30 mL×3) and the combined organic layers washed with brine (10 mL), dried over Na₂SO₄, filtered and concentrated under reduced pressure. The residue was purified by prep-HPLC to afford N-(6-(1-cyanocyclopropyl)imidazo[2,1-b][1,3,4]thiadiazol-2-yl)-4-(2-methoxyphenyl)-6-methylnicotinamide (14 mg, 7%) as a yellow solid. LCMS (ESI, m/z): 431 [M+H]⁺, ¹H NMR (400 MHz, DMSO-d₆): δ 13.0 (s, 1H), 8.72 (s, 1H), 7.96 (s, 1H), 7.44-7.32 (m, 2H), 7.28 (s, 1H), 7.11-7.03 (m, 1H), 7.01-6.95 (m, 1H), 3.53 (s, 3H), 2.56 (s, 3H), 1.68-1.61 (m, 2H), 1.51-1.45 (m, 2H).

TABLE W
The following examples were prepared using a similar procedure to that described for Example 166

| Ex. No. | Structure | Compound Name | Characterization Data |
|---|---|---|---|
| 167 | 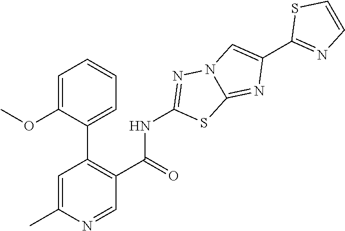 [SMILES: COc1ccccc1-c1cc(C)ncc1C(=O)Nc1nn2cc(-c3nccs3)nc2s1] | 4-(2-Methoxyphenyl)- 6-methyl-N-(6-(thiazol- 2-yl)imidazo[2,1-b] [1,3,4]thiadiazol-2-yl)- nicotinamide | LCMS (m/z): 449 [M + H]⁺. ¹H NMR (400 MHz, DMSO- d₆) δ 13.2 (s, 1H), 8.72 (s, 1H), 8.54 (s, 1H), 7.83 (d, J = 3.2 Hz, 1H), 7.65 (d, J = 3.2 Hz, 1H), 7.44-7.37 (m, 2H), 7.35 (s, 1H), 7.13-7.07 (m, 1H), 7.01 (d, J = 8.2 Hz, 1H), 3.56 (s, 3H), 2.59 (s, 3H) |
| 168 | 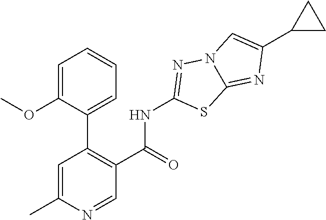 [SMILES: COc1ccccc1-c1cc(C)ncc1C(=O)Nc1nn2cc(C3CC3)nc2s1] | N-(6-cyclopropylimi- dazo[2,1-b][1,3,4] thiadiazol-2-yl)-4-(2- methoxyphenyl)-6- methylnicotinamide | LCMS (m/z): 406 [M + H]⁺. ¹H NMR (300 MHz, DMSO- d₆) δ 12.96 (s, 1H), 8.72 (s, 1H), 7.62 (s, 1H), 7.39-7.25 (m, 3H), 7.06-6.96 (m, 2H), 3.67 (s, 3H), 2.55 (s, 3H), 1.86 (s, 1H), 0.79 (d, J = 8.4 Hz, 2H), 0.69 (s, 2H) |
| 169 | 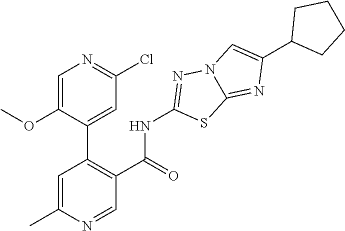 [SMILES: COc1cnc(Cl)cc1-c1cc(C)ncc1C(=O)Nc1nn2cc(C3CCCC3)nc2s1] | 2′-chloro-N-(6-cyclo- pentylimidazo[2,1-b] [1,3,4]thiadiazol-2-yl)- 5′-methoxy-6-methyl- [4,4′-bipyridine]-3- carboxamide | LCMS (m/z): 469 [M + H]⁺. ¹H NMR (400 MHz, DMSO- d₆) δ 13.13 (s, 1H), 8.85 (s, 1H), 8.18 (s, 1H), 7.73 (s, 1H), 7.55 (s, 1H), 7.44 (s, 1H), 3.65 (s, 3H), 3.09-3.00 (m, 1H), 2.59 (s, 3H), 2.00- 1.89 (m, 2H), 1.73-1.57 (m, 6H) |
| 170 | 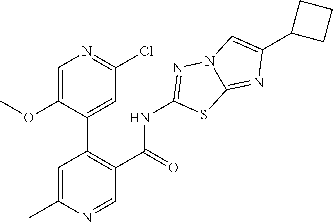 [SMILES: COc1cnc(Cl)cc1-c1cc(C)ncc1C(=O)Nc1nn2cc(C3CCC3)nc2s1] | 2′-chloro-N-(6-cyclo- butylimidazo[2,1-b] [1,3,4]thiadiazol-2-yl)- 5′-methoxy-6-methyl- [4,4′-bipyridine]-3- carboxamide | LCMS (m/z): 455 [M + H]⁺. ¹H NMR (400 MHz, DMSO- d₆) δ 13.13 (s, 1H), 8.84 (s, 1H), 8.18 (s, 1H), 7.77 (s, 1H), 7.56 (s, 1H), 7.45 (s, 1H), 3.66 (s, 3H), 3.48 (d, J = 8.4 Hz, 1H), 2.60 (s, 3H), 2.30-2.11 (m, 4H), 1.94 (m, 1H), 1.89-1.79 (m, 1H) |
| 171 | 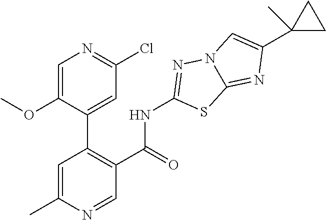 [SMILES: COc1cnc(Cl)cc1-c1cc(C)ncc1C(=O)Nc1nn2cc(C3(C)CC3)nc2s1] | 2′-chloro-5′-methoxy- 6-methyl-N-(6-(1- methylcyclopropyl)- imidazo[2,1-b][1,3,4] thiadiazol-2-yl)-[4,4′- bipyridine]-3-carbox- amide | LCMS (m/z): 455 [M + H]⁺. ¹H NMR (400 MHz, DMSO- d₆) δ 13.11 (s, 1H), 8.84 (s, 1H), 8.18 (s, 1H), 7.73 (s, 1H), 7.56 (s, 1H), 7.44 (s, 1H), 3.65 (s, 3H), 2.59 (s, 3H), 1.38 (s, 3H), 0.99-0.89 (m, 2H), 0.68-0.63 (m, 2H) |
| 172^{&,c} | 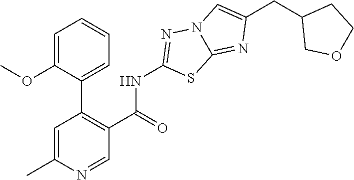 [SMILES: COc1ccccc1-c1cc(C)ncc1C(=O)Nc1nn2cc(CC3CCOC3)nc2s1] | (R or S)-4-(2-methoxy- phenyl)-6-methyl-N- (6-((tetrahydrofuran- 3-yl)methyl)imidazo [2,1-b][1,3,4]thiadia- zol-2-yl)nicotinamide | LCMS (m/z): 450 [M + H]⁺. ¹H NMR (300 MHz, Chloro- form-d) δ 9.34 (s, 1H), 7.53- 7.48 (m, 2H), 7.38-7.36 (m, 2H), 7.17-7.13 (t, J = 7.2 Hz, 1H), 6.93-6.85 (m, 1H), 3.84- 3.65 (m, 6H), 3.48-3.43 (m, 1H), 2.82 (s, 5H), 2.68-2.59 (m, 1H), 2.12-2.02 (m, 1H), 1.65-1.55 (m, 1H) |
| 173 | 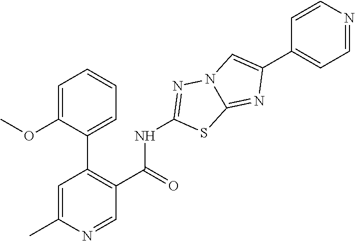 [SMILES: COc1ccccc1-c1cc(C)ncc1C(=O)Nc1nn2cc(-c3ccncc3)nc2s1] | 4-(2-methoxyphenyl)- 6-methyl-N-(6-(pyr- idin-4-yl)imidazo[2,1- b][1,3,4]thiadiazol-2- yl)nicotinamide | LCMS (m/z): 443 [M + H]⁺. ¹H NMR (400 MHz, DMSO- d₆) δ 13.16 (s, 1H), 8.75 (s, 2H), 8.54 (d, J = 5.2 Hz, 2H), 7.80-7.74 (m, 2H), 7.50-7.21 (m, 3H), 7.09 (s, 1H), 7.01 (s, 1H), 3.56 (s, 3H), 2.57 (s, 3H) |
| 174^{&,d} | 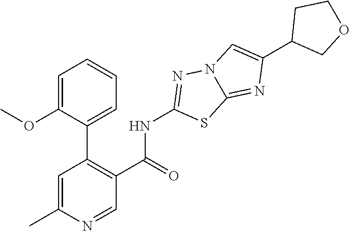 [SMILES: COc1ccccc1-c1cc(C)ncc1C(=O)Nc1nn2cc(C3CCOC3)nc2s1] | (R or S)-4-(2-methoxy- phenyl)-6-methyl-N- (6-(tetrahydrofuran- 3-yl)imidazo[2,1-b] [1,3,4]thiadiazol-2- yl)nicotinamide | LCMS (m/z): 436 [M + H]⁺. ¹H NMR (400 MHz, DMSO- d₆) δ 12.94 (s, 1H), 8.69 (s, 1H), 7.83 (s, 1H), 7.45-7.35 (m, 3H), 7.33 (s, 1H), 7.13- 7.05 (m, 1H), 4.00-3.96 (m, 1H), 3.89-3.73 (m, 2H), 3.66 (t, J = 7.6 Hz, 1H), 3.54 (s, 3H), 3.44-3.36 (m, 1H), 2.58 (s, 3H), 2.24-2.16 (m, 1H), 2.11-2.02 (m, 2H) |
| 175^{&,e} | 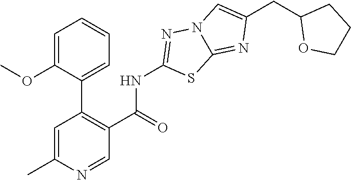 [SMILES: COc1ccccc1-c1cc(C)ncc1C(=O)Nc1nn2cc(CC3CCCO3)nc2s1] | (S or R)-4-(2-methoxy- phenyl)-6-methyl-N- (6-((tetrahydrofuran- 2-yl)methyl)imidazo [2,1-b][1,3,4]thiadia- zol-2-yl)nicotinamide | LCMS (m/z): 450 [M + H]⁺. ¹H NMR (400 MHz, DMSO- d₆) δ 12.94 (s, 1H), 8.70 (s, 1H), 7.61 (s, 1H), 7.40-7.38 (m, 2H), 7.33 (s, 1H), 7.11- 7.07 (m, 1H), 7.00-6.99 (m, 1H), 4.09-4.03 (m, 1H), 3.80- 3.74 (m, 1H), 3.62-3.57 (m, 1H), 3.54 (s, 3H), 2.82-2.76 (m, 1H), 2.69-2.63 (m, 1H), 2.58 (s, 3H), 1.96-1.79 (m, 3H), 1.60-1.54 (m, 1H) |
| 176^{&,c} | 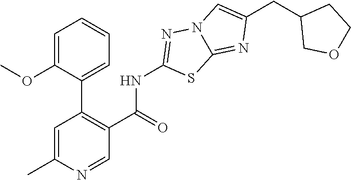 [SMILES: COc1ccccc1-c1cc(C)ncc1C(=O)Nc1nn2cc(CC3CCOC3)nc2s1] | (S or R)-4-(2-methoxy- phenyl)-6-methyl-N- (6-((tetrahydrofuran-3- yl)methyl)imidazo[2,1- b][1,3,4]thiadiazol-2- yl)nicotinamide | LCMS (m/z): 450 [M + H]⁺. ¹H NMR (300 MHz, Chloro- form-d) δ 9.23 (s, 1H), 7.52- 7.48 (m, 2H), 7.37-7.35 (m, 2H), 7.17-7.12 (t, J = 7.2 Hz, 1H), 6.94-6.88 (m, 1H), 3.85- 3.74 (m, 3H), 3.72-3.64 (m, 3H), 3.64-3.45 (m, 1H), 2.79 (s, 5H), 2.64-2.59 (m, 1H), 2.12-2.02 (m, 1H), 1.65-1.55 (m, 1H) |
| 177^{&,d} | 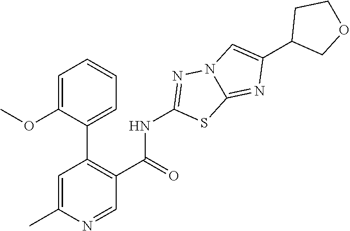 [SMILES: COc1ccccc1-c1cc(C)ncc1C(=O)Nc1nn2cc(C3CCOC3)nc2s1] | (S or R)-4-(2-meth- oxyphenyl)-6-methyl- N-(6-(tetrahydrofuran- 3-yl)imidazo[2,1-b] [1,3,4]thiadiazol-2-yl)- nicotinamide | LCMS (m/z): 436 [M + H]⁺. ¹H NMR (400 MHz, DMSO- d₆) δ 12.94 (s, 1H), 8.69 (s, 1H), 7.83 (s, 1H), 7.45-7.35 (m, 3H), 7.11-6.98 (s, 2H), 4.00-3.96 (m, 1H), 3.89-3.73 (m, 2H), 3.70-3.62 (m, 1H), 3.54 (s, 3H), 3.44-3.36 (m, 1H), 2.58 (s, 3H), 2.24-2.16 (m, 1H), 2.11-2.02 (m, 1H) |
| 178 | 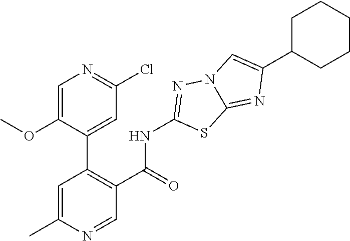 [SMILES: COc1cnc(Cl)cc1-c1cc(C)ncc1C(=O)Nc1nn2cc(C3CCCCC3)nc2s1] | 2′-chloro-N-(6-cyclo- hexylimidazo[2,1-b] [1,3,4]thiadiazol-2-yl)- 5′-methoxy-6-methyl- [4,4′-bipyridine]-3- carboxamide | LCMS (m/z): 483 [M + H]⁺. ¹H NMR (300 MHz, DMSO- d₆) δ 13.15 (s, 1H), 8.85 (s, 1H), 8.18 (s, 1H), 7.80-7.29 (m, 3H), 3.66 (s, 3H), 2.60 (s, 4H), 2.16-1.87 (m, 2H), 1.87-1.56 (m, 3H), 1.51-0.69 (m, 5H) |
| 179 | 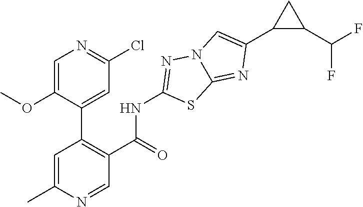 [SMILES: COc1cnc(Cl)cc1-c1cc(C)ncc1C(=O)Nc1nn2cc(C3CC3C(F)F)nc2s1] | (Racemic)-2′-chloro- N-(6-(2-(difluorometh- yl)cyclopropyl)imidazo [2,1-b][1,3,4]thiadia- zol-2-yl)-5′-methoxy- 6-methyl-[4,4′-bipyr- idine]-3-carboxamide | LCMS (m/z): 491 [M + H]⁺. ¹H NMR (400 MHz, DMSO- d₆) δ 13.16 (s, 1H), 8.86 (s, 1H), 8.17 (s, 1H), 7.86-7.77 (m, 1H), 7.51-7.39 (m, 2H), 5.93-5.88 (m, 1H), 3.67-3.63 (m, 3H), 2.58 (s, 3H), 2.33 (d, J = 8.0 Hz, 1H), 2.20 (s, 1H), 1.76 (s, 1H), 1.73-1.65 (m, 1H) |
| 180 | 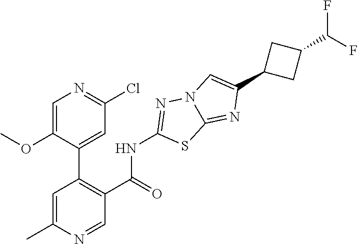 [SMILES: COc1cnc(Cl)cc1-c1cc(C)ncc1C(=O)Nc1nn2cc([C@H]3C[C@H](C(F)F)C3)nc2s1] | 2′-chloro-N-(6-((1r,3r)- 3-(difluoromethyl)- cyclobutyl)imidazo [2,1-b][1,3,4]thiadia- zol-2-yl)-5′-methoxy- 6-methyl-[4,4′-bipyr- idine]-3-carboxamide | LCMS (m/z): 505 [M + H]⁺. ¹H NMR (400 MHz, DMSO- d₆) δ 13.13 (s, 1H), 8.87 (s, 1H), 8.16-8.13 (m, 1H), 7.72 (s, 1H), 7.52 (s, 1H), 7.39 (s, 1H), 6.16-6.07 (m, 1H), 3.65 (s, 3H), 3.44-3.42 (m, 1H), 2.78-2.64 (m, 1H), 2.58 (s, 3H), 2.29 (d, J = 8.3 Hz, 2H), 2.17 (d, J = 8.6 Hz, 2H) |
| 181 | 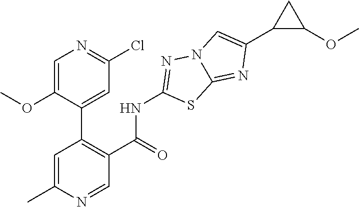 | (Racemic)-2′-chloro- 5′-methoxy-N-(6-(2- methoxycyclopropyl)- imidazo[2,1-b][1,3,4] thiadiazol-2-yl)-6- methyl-[4,4′-bipyr- idine]-3-carboxamide | LCMS (m/z): 471 [M + H]⁺. ¹H NMR (400 MHz, DMSO- d₆) δ 9.07 (s, 1H), 8.11 (s, 1H), 7.96 (s, 1H), 7.35 (s, 1H), 7.19 (s, 1H), 4.64 (d, J = 5.3 Hz, 1H), 4.44-4.24 (m, 2H), 3.61 (s, 3H), 3.31 (s, 3H), 3.26-3.24 (m, 1H), 2.54 (s, 3H) |
| 182 | 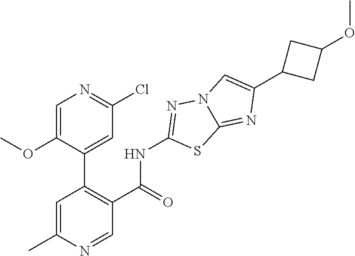 | (Cis and Trans)-2′- chloro-5′-methoxy-N- (6-(3-methoxycyclo- butyl)imidazo[2,1-b] [1,3,4]thiadiazol-2-yl)- 6-methyl-[4,4′-bipyr- idine]-3-carboxamide | LCMS (m/z): 485 [M + H]⁺. ¹H NMR (400 MHz, DMSO- d₆) δ 8.92 (s, 1H), 8.14 (s, 1H), 7.58 (s, 1H), 7.44 (s, 1H), 7.29 (s, 1H), 3.77 (t, J = 7.2 Hz, 1H), 3.64 (s, 3H), 3.14 (s, 3H), 2.96-2.87 (m, 1H), 2.56 (s, 3H), 2.00 (d, J = 8.5 Hz, 2H), 1.24 (s, 2H) |
| 183 | 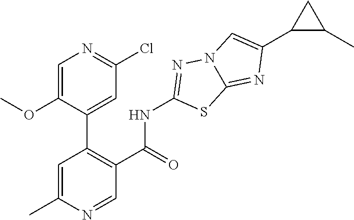 | (Racemic)-2′-chloro- 5′-methoxy-6-methyl- N-(6-(2-methylcyclo- propyl)imidazo[2,1-b] [1,3,4]thiadiazol-2-yl)- [4,4′-bipyridine]-3- carboxamide | LCMS (m/z): 455 [M + H]⁺. ¹H NMR (400 MHz, DMSO- d₆) δ 8.92 (s, 1H), 8.01 (s, 1H), 7.67 (s, 1H), 7.41 (s, 1H), 7.29 (s, 1H), 4.45-4.40 (m, 1H), 3.90-3.85 (m, 1H), 3.69 (s, 3H), 3.29-3.21 (m, 2H), 2.78 (d, J = 1.7 Hz, 1H), 2.64 (s, 1H), 1.61-1.59 (m, 1H), 1.34-1.29 (m, 6H), 0.91-0.89 (m, 1H) |
| 184 | 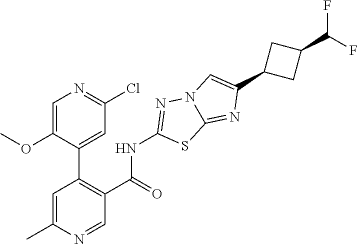 [SMILES: COc1cnc(Cl)cc1-c1cc(C)ncc1C(=O)Nc1nn2cc([C@H]3C[C@@H](C(F)F)C3)nc2s1] | 2′-chloro-N-(6-((1s, 3s)-3-(difluorometh- yl)cyclobutyl)imidazo [2,1-b][1,3,4]thiadia- zol-2-yl)-5′-methoxy- 6-methyl-[4,4′-bipyr- idine]-3-carboxamide | LCMS (m/z): 505 [M + H]⁺. ¹H NMR (400 MHz, DMSO- d₆) δ 13.15 (s, 1H), 8.87 (s, 1H), 8.17-8.13 (m, 1H), 7.77 (s, 1H), 7.53 (s, 1H), 7.40 (s, 1H), 6.29 (s, 1H), 6.19 (s, 1H), 3.65 (s, 3H), 3.56-3.49 (m, 1H), 2.77-2.73 (m, 1H), 2.58 (s, 3H), 2.31 (t, J = 7.6 Hz, 4H) |
| 185 | 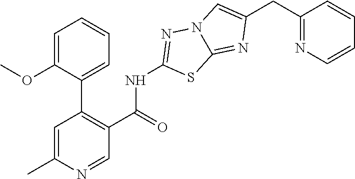 [SMILES: COc1ccccc1-c1cc(C)ncc1C(=O)Nc1nn2cc(Cc3ccccn3)nc2s1] | 4-(2-methoxyphenyl)- 6-methyl-N-(6-(pyr- idin-2-ylmethyl)imi- dazo[2,1-b][1,3,4]thia- diazol-2-yl)nicotin- amide | LCMS (m/z): 457 [M + H]⁺. ¹H NMR (400 MHz, DMSO- d₆) δ 9.01 (m, 2H), 8.66 (m, 1H), 8.39 (d, J = 8.2 Hz, 1H), 8.06 (m, 3H), 7.63 (m, 2H), 7.25 (m, 1H), 7.16-7.02 (m, 1H), 3.60 (d, J = 3.5 Hz, 3H), 2.88 (d, J = 4.0 Hz, 3H), 2.62 (s, 2H) |
| 186 | 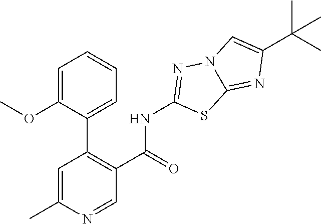 [SMILES: COc1ccccc1-c1cc(C)ncc1C(=O)Nc1nn2cc(C(C)(C)C)nc2s1] | N-(6-(tert-butyl)imi- dazo[2,1b][1,3,4]thia- diazo1-2-yl)-4-(2- methoxyphenyl)-6- methylnicotinamide | LCMS (m/z): 422 [M + H]⁺. ¹H NMR (400 MHz, DMSO- d₆) δ 12.90 (s, 1H), 8.69 (s, 1H), 7.65 (s, 1H), 7.43-7.26 (m, 3H), 7.10-7.08 (m, 1H), 7.00-6.98 (m, 1H), 3.54 (s, 3H), 2.57 (s, 3H), 1.26 (s, 9H) |
| 187 | 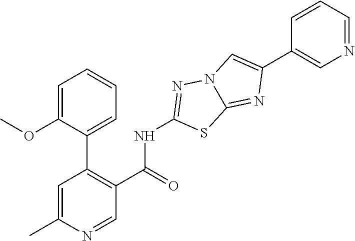 [SMILES: COc1ccccc1-c1cc(C)ncc1C(=O)Nc1nn2cc(-c3cccnc3)nc2s1] | 4-(2-methoxyphenyl)- 6-methyl-N-(6-(pyr- idin-3-yl)imidazo[2,1- b][1,3,4]thiadiazol-2- yl)nicotinamide | LCMS (m/z): 443 [M + H]⁺. ¹H NMR (300 MHz, DMSO- d₆) δ 13.12 (s, 1H), 9.06 (s, 1H), 8.73 (s, 1H), 8.65 (s, 1H), 8.45 (d, J = 4.8 Hz, 1H), 8.19-8.14 (m, 1H), 7.44-7.34 (m, 1H), 7.10 (t, J = 7.5 Hz, 1H), 7.01 (d, J = 8.1 Hz, 1H), 3.56 (s, 3H), 2.59 (s, 3H), 1.24 (s, 1H) |
| 188 | 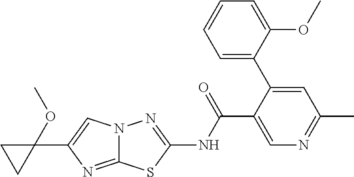 [SMILES: COc1ccccc1-c1cc(C)ncc1C(=O)Nc1nn2cc(C3(OC)CC3)nc2s1] | N-(6-(1-methoxy- cyclopropyl)imidazo [2,1-b][1,3,4]thiadia- zol-2-yl)-4-(2-meth- oxyphenyl)-6-methyl- nicotinamide | LCMS (m/z): 436.00 [M + H]⁺. ¹H NMR (300 MHz, DMSO-d₆) δ 13.03 (s, 1H), 8.75 (s, 1H), 7.96 (s, 1H), 7.39-7.42 (m, 3H), 7.00-7.10 (m, 2H), 3.74-3.72 (m, 1H), 3.55 (s, 3H), 3.28 (s, 3H), 2.60 (s, 3H), 0.84-0.85 (m, 2H), 1.07-1.02 (m, 2H) |
| 189^{&,f} | 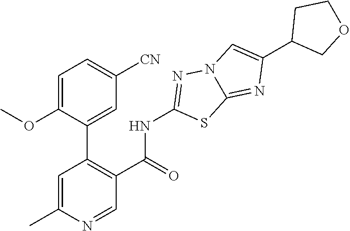 [SMILES: COc1ccc(C#N)cc1-c1cc(C)ncc1C(=O)Nc1nn2cc(C3CCOC3)nc2s1] | (S or R)-4-(5-cyano- 2-methoxyphenyl)-6- methyl-N-(6-(tetra- hydrofuran-3-yl)imi- dazo[2,1-b][1,3,4]thia- diazol-2-yl)nicotin- amide | LCMS (m/z): 461 [M + H]⁺. ¹H NMR (300 MHz, Meth- anol-d₄) δ 8.73 (s, 1H), 7.81 (d, J = 7.8 Hz, 2H), 7.64 (s, 1H), 7.42 (s, 1H), 7.15 (d, J = 8.6 Hz, 1H), 4.09 (t, J = 7.8 Hz, 1H), 4.05-3.84 (m, 2H), 3.81-3.74 (m, 1H), 3.70 (s, 3H), 3.61-3.42 (m, 1H), 2.66 (s, 3H), 2.32 (m, 1H), 2.22- 2.03 (m, 1H), 1.33-1.28 (m, 5H), 0.88 (d, J = 7.3 Hz, 2H) |
| 190^{&,g} | 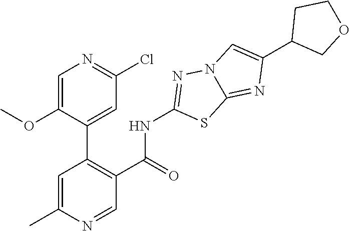 [SMILES: COc1cnc(Cl)cc1-c1cc(C)ncc1C(=O)Nc1nn2cc(C3CCOC3)nc2s1] | (S or R)-2′-chloro-5′- methoxy-6-methyl-N- (6-(tetrahydrofuran-3- yl)imidazo[2,1-b] [1,3,4]thiadiazol-2-yl)- [4,4′-bipyridine]-3- carboxamide | LCMS (m/z): 471 [M + H]⁺. ¹H NMR (300 MHz, DMSO- d₆) δ 8.86 (s, 1H), 8.15 (d, J = 9.2 Hz, 1H), 7.77 (s, 1H), 7.53 (s, 1H), 7.40 (s, 1H), 3.98 (t, J = 7.7 Hz, 1H), 3.88- 3.73 (m, 2H), 3.65 (s, 4H), 3.41 (d, J = 7.5 Hz, 1H), 2.58 (s, 3H), 2.28-1.97 (m, 2H) |
| 191^{&,f} | 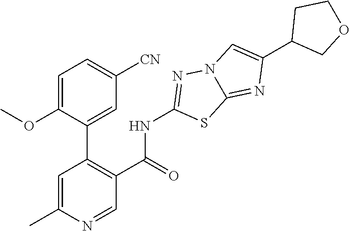 [SMILES: COc1ccc(C#N)cc1-c1cc(C)ncc1C(=O)Nc1nn2cc(C3CCOC3)nc2s1] | (R or S)-4-(5-cyano- 2-methoxyphenyl)-6- methyl-N-(6-(tetra- hydrofuran-3-yl)imi- dazo[2,1-b][1,3,4] thiadiazol-2-yl)nico- tinamide | LCMS (m/z): 461 [M + H]⁺. ¹H NMR (300 MHz, Meth- anol-d₄) δ 8.73 (s, 1H), 7.81 (d, J = 7.9 Hz, 2H), 7.63 (s, 1H), 7.42 (s, 1H), 7.15 (d, J = 8.4 Hz, 1H), 4.08 (d, J = 7.8 Hz, 1H), 4.01-3.80 (m, 2H), 3.76 (d, J = 7.8 Hz, 1H), 3.70 (s, 3H), 3.52 (d, J = 7.6 Hz, 1H), 2.66 (s, 3H), 2.31-2.16 (m, 1H), 2.13-2.03 (m, 1H), 1.33-1.28 (m, 5H), 0.89-087 (m, 2H) |
| 192 | 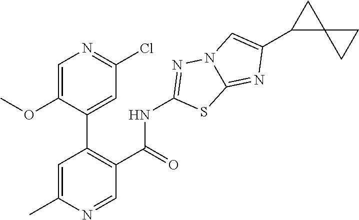 [SMILES: COc1cnc(Cl)cc1-c1cc(C)ncc1C(=O)Nc1nn2cc(C3CC34CC4)nc2s1] | (Racemic)-2′-chloro- 5′-methoxy-6-methyl- N-(6-(spiro[2.2]pentan- 1-yl)imidazo[2,1-b] [1,3,4]thiadiazol-2-yl)- [4,4′-bipyridine]-3- carboxamide | LCMS (m/z): 467 [M + H]⁺. ¹H NMR (300 MHz, DMSO- d₆) δ 13.19 (s, 1H), 8.87 (s, 1H), 8.18 (s, 1H), 7.65 (s, 1H), 7.53 (s, 1H), 7.40 (s, 1H), 6.00 (t, J = 2.4 Hz, 1H), 3.66 (s, 3H), 2.96-2.93 (m, 2H), 2.84-2.81 (m, 2H), 2.59 (s, 3H), 2.06-2.03 (m, 2H) |
| 193 | 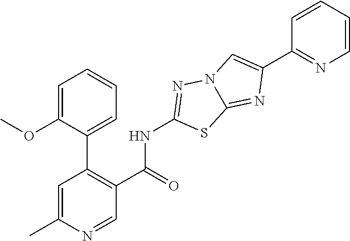 [SMILES: COc1ccccc1-c1cc(C)ncc1C(=O)Nc1nn2cc(-c3ccccn3)nc2s1] | 4-(2-methoxyphenyl)- 6-methyl-N-(6-(pyr- idin-2-yl)imidazo[2,1- b][1,3,4]thiadiazol-2- yl)nicotinamide | LCMS (m/z): 443 [M + H]⁺. ¹H NMR (400 MHz, DMSO- d₆) δ 13.11 (s, 1H), 8.74 (s, 1H), 8.62-8.48 (m, 1H), 8.43 (s, 1H), 8.03-7.88 (m, 1H), 7.84-7.82 (m, 1H), 7.41-7.39 (m, 2H), 7.32 (s, 1H), 7.30- 7.20 (m, 1H), 7.09-7.07 (m, 1H), 7.01 (d, J = 8.2 Hz, 1H), 3.57 (s, 3H), 2.58 (s, 3H) |
| 194 | 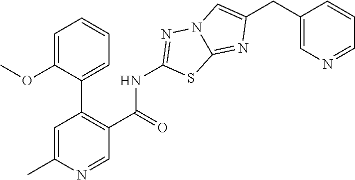 [SMILES: COc1ccccc1-c1cc(C)ncc1C(=O)Nc1nn2cc(Cc3cccnc3)nc2s1] | 4-(2-methoxyphenyl)- 6-methyl-N-(6-(pyr- idin-3-ylmethyl)- imidazo[2,1-b][1,3,4] thiadiazol-2-yl)nico- tinamide | LCMS (m/z): 457 [M + H]⁺. ¹H NMR (300 MHz, DMSO- d₆) δ 12.96 (s, 1H), 8.70 (s, 1H), 8.52 (s, 1H), 8.40 (d, J = 5.0 Hz, 1H), 7.75-7.67 (m, 2H), 7.41-7.29 (m, 4H), 7.07 (t, J = 7.5 Hz, 1H), 6.97 (d, J = 8.2 Hz, 1H), 3.93 (s, 2H), 3.52 (s, 3H), 2.57 (s, 3H) |
| 195^{&,e} | 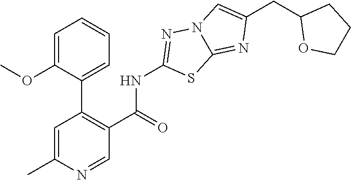 [SMILES: COc1ccccc1-c1cc(C)ncc1C(=O)Nc1nn2cc(CC3CCCO3)nc2s1] | (R or S)-4-(2-methoxy- phenyl)-6-methyl-N- (6-((tetrahydrofuran- 2-yl)methyl)imidazo [2,1-b][1,3,4]thiadia- zol-2-yl)nicotinamide | LCMS (m/z): 450 [M + H]⁺. ¹H NMR (400 MHz, DMSO- d₆) δ 12.94 (s, 1H), 8.70 (s, 1H), 7.37 (s, 1H), 7.39-7.25 (m, 3H), 7.07-7.03 (m, 1H), 6.99-6.97 (m, 1H), 4.08-4.02 (m, 1H), 3.86-3.72 (m, 1H), 3.62-3.56 (m, 1H), 3.54 (s, 3H), 2.80-2.75 (m, 1H), 2.66- 2.60 (m, 1H), 2.55 (s, 3H), 1.94-1.74 (m, 3H), 1.60-1.52 (m, 1H) |
| 196 | 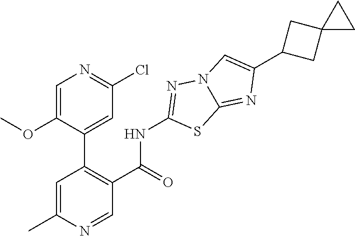 [SMILES: COc1cnc(Cl)cc1-c1cc(C)ncc1C(=O)Nc1nn2cc(C3CC4(CC4)C3)nc2s1] | 2′-chloro-5′-methoxy- 6-methyl-N-(6-(spiro [2.3]hexan-5-yl)imi- dazo[2,1-b][1,3,4] thiadiazol-2-yl)-[4,4′- bipyridine]-3-carbox- amide | LCMS (m/z): 481 [M + H]⁺. ¹H NMR (400 MHz, DMSO- d₆) δ 13.04 (s, 1H), 8.90 (s, 1H), 8.15 (d, J = 7.0 Hz, 1H), 7.66-7.47 (m, 2H), 7.34 (s, 1H), 3.65-3.63 (m, 4H), 2.78- 2.79 (m, 3H), 2.32-2.23 (m, 2H), 2.27-2.25 (m, 2H), 1.23- 1.24 (m, 1H), 0.48-0.39 (m, 3H) |
| 197^{&,g} | 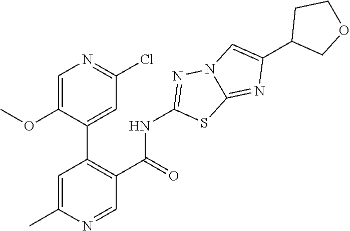 [SMILES: COc1cnc(Cl)cc1-c1cc(C)ncc1C(=O)Nc1nn2cc(C3CCOC3)nc2s1] | (R or S)-2′-chloro-5′- methoxy-6-methyl-N- (6-(tetrahydrofuran- 3-yl)imidazo[2,1-b] [1,3,4]thiadiazol-2-yl)- [4,4′-bipyridine]-3- carboxamide | LCMS (m/z): 471 [M + H]⁺. ¹H NMR (300 MHz, DMSO- d₆) δ 13.15 (s, 1H), 8.84 (s, 1H), 8.18 (s, 1H), 7.85 (s, 1H), 7.52 (m, 2H), 3.98 (t, J = 7.7 Hz, 1H), 3.83 (s, 2H), 3.66 (s, 4H), 2.60 (s, 3H), 2.31-2.02 (m, 2H) |
| 198 | 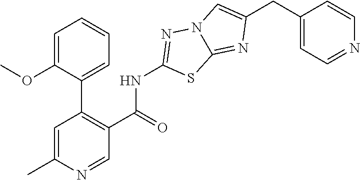 [SMILES: COc1ccccc1-c1cc(C)ncc1C(=O)Nc1nn2cc(Cc3ccncc3)nc2s1] | 4-(2-methoxyphenyl)- 6-methyl-N-(6-(pyr- idin-4-ylmethyl)imi- dazo[2,1-b][1,3,4] thiadiazol-2-yl)nico- tinamide | LCMS (m/z): 457 [M + H]⁺. ¹H NMR (400 MHz, DMSO- d₆) δ 12.96 (s, 1H), 8.70 (s, 1H), 8.51 (d, J = 5.0 Hz, 2H), 7.85 (s, 1H), 7.39-7.34 (m, 5H), 7.10 (t, J = 7.4 Hz, 1H), 6.99 (d, J = 8.2 Hz, 1H), 3.99 (s, 2H), 3.53 (s, 3H), 2.58 (s, 3H) |
| 199 | 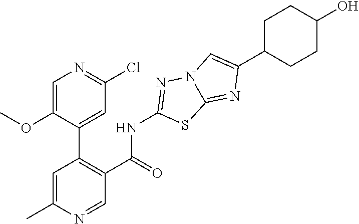 [SMILES: COc1cnc(Cl)cc1-c1cc(C)ncc1C(=O)Nc1nn2cc(C3CCC(O)CC3)nc2s1] | (Cis and Trans)-2′- chloro-N-(6-(4- hydroxycyclohexyl)- imidazo[2,1-b][1,3,4] thiadiazol-2-yl)-5′- methoxy-6-methyl- [4,4′-bipyridine]-3- carboxamide | LCMS (m/z): 499 [M + H]⁺. ¹H NMR (400 MHz, DMSO- d₆) δ 13.10 (s, 1H), 8.83 (s, 1H), 8.18 (s, 1H), 7.72 (s, 1H), 7.57 (s, 1H), 7.46 (s, 1H), 4.57 (s, 1H), 3.65 (s, 3H), 3.43 (d, J = 5.8 Hz, 2H), 2.60 (s, 3H), 2.06-1.84 (m, 4H), 1.47-1.33 (m, 2H), 1.30- 1.21 (m, 2H) |

^{&}Single enantiomer, unknown stereochemistry
^{c}Chiral separation conditions of Example 172 and 176: Column: CHIRALPAK IC-3, 4.6 × 50 mm, 3 μm; Mobile Phase A: MTBE (0.1%TFA): MeOH = 75:25; Flow rate: 1.00 mL/min; Peak number: 2; retention time: 3.206 min (Example 176), 4.016 min (Example 172)
^{d}Chiral separation conditions of Example 174 and 177: Column: CHIRALPAK IC-3, 4.6 × 50 mm, 3 μm; Mobile Phase A: MTBE (0.1%TFA): MeOH = 70:30; Flow rate: 1.00 mL/min; Peak number: 2; retention time: 1.794 min (Example 174), 2.499 min (Example 177)
^{e}Chiral separation conditions of Example 175 and 195: Column: CHIRALPAK IC-3, 4.6 × 50 mm, 3 μm; Mobile Phase A: MTBE (0.1%TFA): MEOH = 75:25; Flow rate: 1.00 mL/min; Peak number: 2; retention time: 2.160 min (Example 195), 2.695 min (Example 175)
^{f}Chiral separation conditions of Example 189 and 191: Column: CHIRALPAK ID-3, 4.6 × 50 mm, 3 μm; Mobile Phase A: Hex: MTBE = 1:1 (0.1% TFA): EtOH = 50:50; Flow rate: 1.00 mL/min; Peak number: 2; retention time: 1.663 min (Example 191), 2.802 min (Example 189)
^{g}Chiral separation conditions of Example 190 and 197: Column: CHIRALPAK IA-3, 4.6 ×100 mm, 3 μm; Mobile Phase A: (Hex: MTBE = 1:1) (0.1% TFA): MeOH = 80:20; Flow rate: 1.00 mL/min; Peak number: 2; retention time: 3.115 min (Example 197), 3.420 min (Example 190)

### Example 200: Synthesis of 4-(2-Methoxyphenyl)-6-methyl-N-(6-(pyrimidin-4-yl)imidazo[2,1-b][1,3,4]thiadiazol-2-yl)nicotinamide

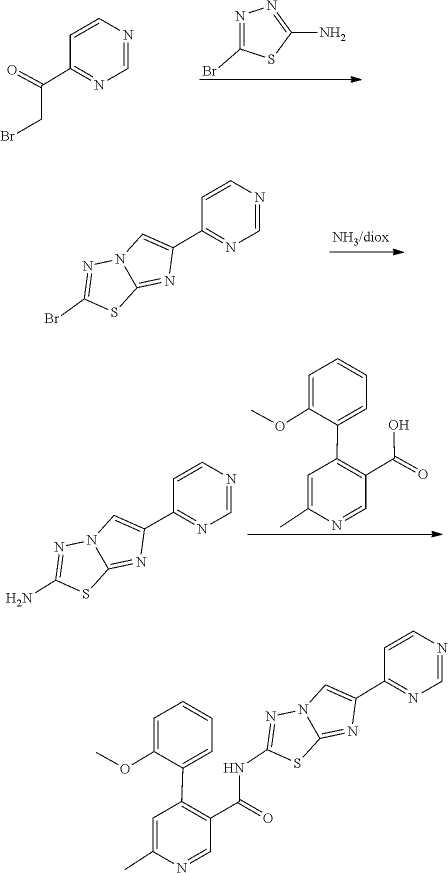 [SMILES: O=C(CBr)c1ccncn1 ; Nc1nnc(Br)s1 ; Brc1nn2cc(-c3ccncn3)nc2s1 ; Nc1nn2cc(-c3ccncn3)nc2s1 ; COc1ccccc1-c1cc(C)ncc1C(=O)O ; COc1ccccc1-c1cc(C)ncc1C(=O)Nc1nn2cc(-c3ccncn3)nc2s1]

Step 1: A mixture of 2-bromo-1-(pyrimidin-4-yl)ethan-1-one hydrobromic acid salt (100 mg, 0.355 mmol) and 5-bromo-1,3,4-thiadiazol-2-amine (64.0 mg, 0.355 mmol) in EtOH (5 mL) was heated at 90° C. in a sealed tube overnight. The mixture was concentrated under reduced pressure and the residue purified by prep-TLC (DCM/MeOH=20/1, v/v) to give 2-bromo-6-(pyrimidin-4-yl)imidazo[2,1-b][1,3,4]thiadiazole as a yellow solid (30 mg, 30%). LCMS (ESI, m/z): 282, 284 [M+H]⁺.

Step 2: To a solution of 2-bromo-6-(pyrimidin-4-yl)imidazo[2,1-b][1,3,4]thiadiazole (45 mg, 0.16 mmol) in 1,4-dioxane (4 mL) was added concentrated aqueous NH₄OH (2 mL) and the mixture was heated in a sealed tube at 90° C. overnight. The mixture was concentrated under reduced pressure and the residue purified by prep-TLC (DCM/MeOH=15/1, v/v) to afford 6-(pyrimidin-4-yl)imidazo[2,1-b][1,3,4]thiadiazol-2-amine (20 mg, 45%) as a yellow solid. LCMS (ESI, m/z): 219 [M+H]⁺, ¹H NMR (400 MHz, DMSO-d₆): δ 9.03 (d, J=1.4 Hz, 1H), 8.72 (d, J=5.2 Hz, 1H), 8.40 (s, 1H), 7.81 (dd, J=5.2, 1.4 Hz, 1H), 7.73 (s, 2H).

Step 3: To a solution of 6-(pyrimidin-4-yl)imidazo[2,1-b][1,3,4]thiadiazol-2-amine (43 mg, 0.18 mmol) and 4-(2-methoxyphenyl)-6-methylnicotinic acid (46 mg, 0.21 mmol) in DMF (2 mL) was added EDCI·HCl (51 mg, 0.27 mmol) and DMAP (33 mg, 0.27 mmol) and the mixture was stirred at room temperature overnight. The mixture was diluted with water (30 mL), extracted with EtOAc (2×30 mL) and the combined organic layers washed with water (30 mL), brine (30 mL), dried over Na₂SO₄, filtered and concentrated under reduced pressure. The residue was purified by prep-TLC (DCM/MeOH=15/1, v/v) to afford 4-(2-methoxyphenyl)-6-methyl-N-(6-(pyrimidin-4-yl)imidazo[2,1-b][1,3,4]thiadiazol-2-yl)nicotinamide (14 mg, 15%) as an off-white solid. LCMS (ESI, m/z): 444 [M+H]⁺, ¹H NMR (400 MHz, DMSO-d₆): δ 13.2 (s, 1H), 9.10 (d, J=1.4 Hz, 1H), 8.78 (d, J=5.2 Hz, 1H), 8.73 (d, J=3.0 Hz, 2H), 7.91 (dd, J=5.2, 1.4 Hz, 1H), 7.44-7.37 (m, 2H), 7.34 (s, 1H), 7.09 (t, J=7.4 Hz, 1H), 7.01 (d, J=8.2 Hz, 1H), 3.56 (s, 3H), 2.59 (s, 3H).

### Example 201: Synthesis of 4-(6-Acetyl-2,6-diazaspiro[3.3]heptan-2-yl)-6-methyl-N-(6-(pyridin-4-yl)thiazolo[4,5-b]pyridin-2-yl)nicotinamide

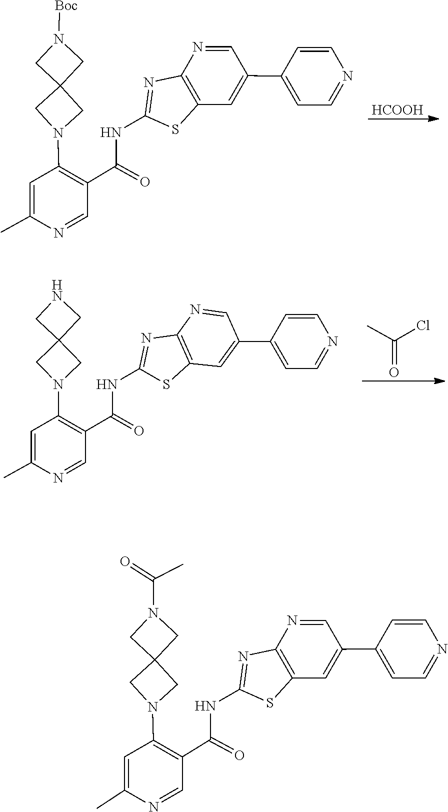

Step 1: A solution of tert-butyl 6-(2-methyl-5-((6-(pyridin-4-yl)thiazolo[4,5-b]pyridin-2-yl)carbamoyl)pyridin-4-yl)-2,6-diazaspiro[3.3]heptane-2-carboxylate (10.0 mg, 0.02 mmol) in formic acid (0.5 mL) was stirred at room temperature for 1 h. The mixture was diluted with water and freeze dried to give 6-methyl-N-(6-(pyridin-4-yl)thiazolo[4,5-b]pyridin-2-yl)-4-(2,6-diazaspiro[3.3]heptan-2-yl)nicotinamide (6.7 mg, 83%) as a yellow solid. LCMS (ESI, m/z): 444 [M+H]⁺.

Step 2: To a solution of 6-methyl-N-(6-(pyridin-4-yl)thiazolo[4,5-b]pyridin-2-yl)-4-(2,6-diazaspiro[3.3]heptan-2-yl)nicotinamide (22.0 mg, 0.05 mmol) in DCM (3 mL) at 0° C. was added DIPEA (19.0 mg, 0.15 mmol) and acetyl chloride (4 mg, 0.05 mmol) and the mixture was stirred at 0° C. for 0.5 h. The mixture was diluted with water (3 mL), extracted with DCM (3×10 mL) and the combined organic phases dried over Na₂SO₄, filtered and concentrated under reduced pressure. The residue was purified by prep-HPLC to give 4-(6-acetyl-2,6-diazaspiro[3.3]heptan-2-yl)-6-methyl-N-(6-(pyridin-4-yl)thiazolo[4,5-b]pyridin-2-yl)nicotinamide (3.5 mg, 15%) as a yellow solid. LCMS (ESI, m/z): 486.10 [M+H]⁺, ¹H NMR (400 MHz, DMSO-d₆): δ 9.14 (d, J=2.2 Hz, 1H), 9.09 (d, J=2.2 Hz, 1H), 8.85 (d, J=5.4 Hz, 2H), 8.63 (s, 1H), 8.13 (d, J=5.4 Hz, 2H), 6.70 (s, 1H), 4.39 (br s, 4H), 4.29 (s, 2H), 4.01 (s, 2H), 2.50 (s, 3H), 1.70 (s, 3H).

### Example 202: Synthesis of 4-(7-Acetyl-2,7-diazaspiro[3.5]nonan-2-yl)-6-methyl-N-(6-(pyridin-4-yl)thiazolo[4,5-b]pyridin-2-yl)nicotinamide

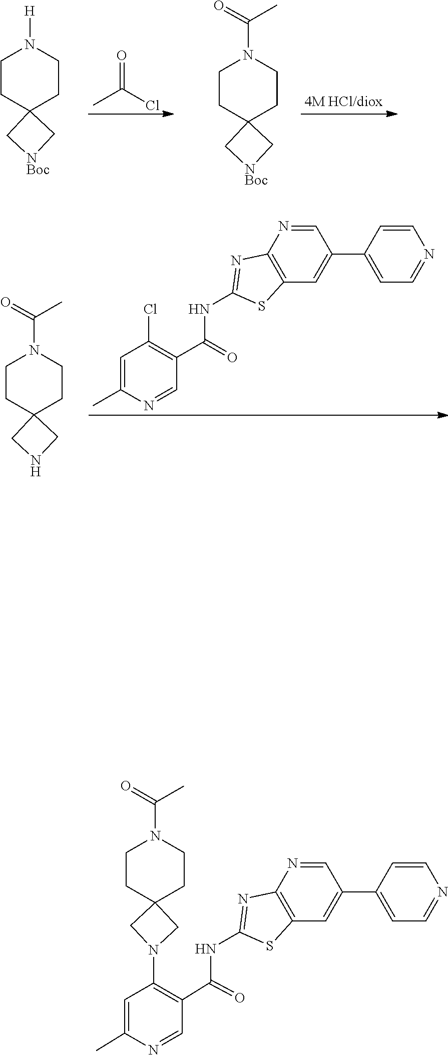

Step 1: To a solution of tert-butyl 2,7-diazaspiro[3.5]nonane-2-carboxylate (200 mg, 0.885 mmol) and TEA (179 mg, 1.77 mmol) in DCM (5 mL) at room temperature was added acetyl chloride (69.5 mg, 0.885 mmol) and the mixture was stirred at room temperature for 2 h. The mixture was diluted with water (50 mL), extracted with DCM (2×50 mL) and the combined organic layers washed with brine (50 mL), dried over Na₂SO₄, filtered and concentrated under reduced pressure to afford tert-butyl 7-acetyl-2,7-diazaspiro[3.5]nonane-2-carboxylate (200 mg, 78%) as a colorless oil. LCMS (ESI, m/z): 291 [M+Na]⁺.

Step 2: A solution of tert-butyl 7-acetyl-2,7-diazaspiro[3.5]nonane-2-carboxylate (200 mg, 0.858 mmol) in 4 M HCl/dioxane (5 mL) and ACN (5 mL) was stirred at room temperature for 1 h. The mixture was concentrated under reduced pressure to afford 1-(2,7-diazaspiro[3.5]nonan-7-yl)ethan-1-one (140 mg) as a colorless oil, which was used in directly in the next step. LCMS (ESI, m/z): 169 [M+H]⁺.

Step 3: To a solution of 1-(2,7-diazaspiro[3.5]nonan-7-yl)ethan-1-one (50 mg, 0.298 mmol) in NMP (1 mL) was added 4-chloro-6-methyl-N-(6-(pyridin-4-yl)thiazolo[4,5-b]pyridin-2-yl)nicotinamide (114 mg, 0.298 mmol) and DIPEA (192 mg, 1.49 mmol) and the mixture was heated at 80° C. overnight. The mixture was diluted with water (20 mL), extracted with EtOAc (2×20 mL) and the combined organic layers washed with brine (20 mL), dried over Na₂SO₄, filtered and concentrated under reduced pressure. The residue was purified by prep-TLC (DCM/MeOH=20/1, v/v) to afford 4-(7-acetyl-2,7-diazaspiro[3.5]nonan-2-yl)-6-methyl-N-(6-(pyridin-4-yl)thiazolo[4,5-b]pyridin-2-yl)nicotinamide (7.5 mg, 5%) as a yellow solid. LCMS (ESI, m/z): 514. [M+H]⁺, ¹H NMR (400 MHz, DMSO-d₆): δ 13.3 (s, 1H), 9.01 (d, J=2.4 Hz, 1H), 8.92 (d, J=2.4 Hz, 1H), 8.69 (d, J=5.2 Hz, 2H), 8.39 (s, 1H), 7.87-7.82 (m, 2H), 6.40 (s, 1H), 3.78-3.70 (m, 4H), 3.44-3.27 (m, 4H, partially obscured by water peak), 2.39 (s, 3H), 1.97 (s, 3H), 1.76-1.70 (m, 2H), 1.66-1.60 (m, 2H).

### Example 203: Synthesis of tert-Butyl 4-(2-methyl-5-((6-(pyridin-4-yl)thiazolo[4,5-b]pyridin-2-yl)carbamoyl)pyridin-4-yl)piperidine-1-carboxylate

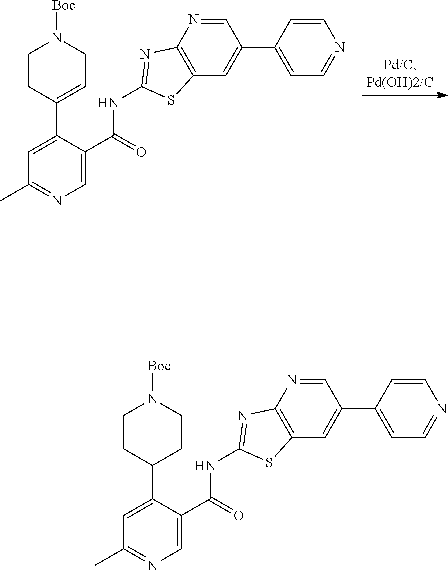 [SMILES: Cc1cc(C2=CCN(C(=O)OC(C)(C)C)CC2)c(C(=O)Nc2nc3ncc(-c4ccncc4)cc3s2)cn1 ; Cc1cc(C2CCN(C(=O)OC(C)(C)C)CC2)c(C(=O)Nc2nc3ncc(-c4ccncc4)cc3s2)cn1]

To a solution of tert-butyl 2′-methyl-5′-((6-(pyridin-4-yl)thiazolo[4,5-b]pyridin-2-yl)carbamoyl)-3,6-dihydro-[4,4′-bipyridine]-1(2H)-carboxylate (40.0 mg, 0.070 mmol) in MeOH (15 mL) was added 10% Pd/C (20 mg) and 20% Pd(OH)₂/C (15 mg) and the mixture was stirred at room temperature under a H₂ atmosphere (2 MPa) for 60 h. The mixture was filtered and the filtrate concentrated under reduced pressure. The residue was purified by prep-HPLC to afford tert-butyl 4-(2-methyl-5-((6-(pyridin-4-yl)thiazolo[4,5-b]pyridin-2-yl)carbamoyl)pyridin-4-yl)piperidine-1-carboxylate (5 mg, 13%) as a white solid. LCMS (ESI, m/z): 531 [M+H]⁺, ¹H NMR (400 MHz, DMSO-d₆): δ 13.3 (s, 1H), 8.99 (d, J=2.2 Hz, 1H), 8.88 (s, 1H), 8.70 (s, 1H), 8.70-8.67 (m, 2H), 7.88-7.80 (m, 2H), 7.39 (s, 1H), 4.16-4.00 (m, 2H), 2.87-2.65 (m, 2H), 2.52 (s, 3H), 1.84-1.72 (m, 2H), 1.64-1.50 (m, 2H), 1.41 (s, 9H). One signal obscured by solvent peak.

### Example 204: Synthesis of 4-(2-Methoxyphenyl)-2-methyl-N-(6-(pyridin-4-yl)thiazolo[4,5-b]pyridin-2-yl)pyrimidine-5-carboxamide

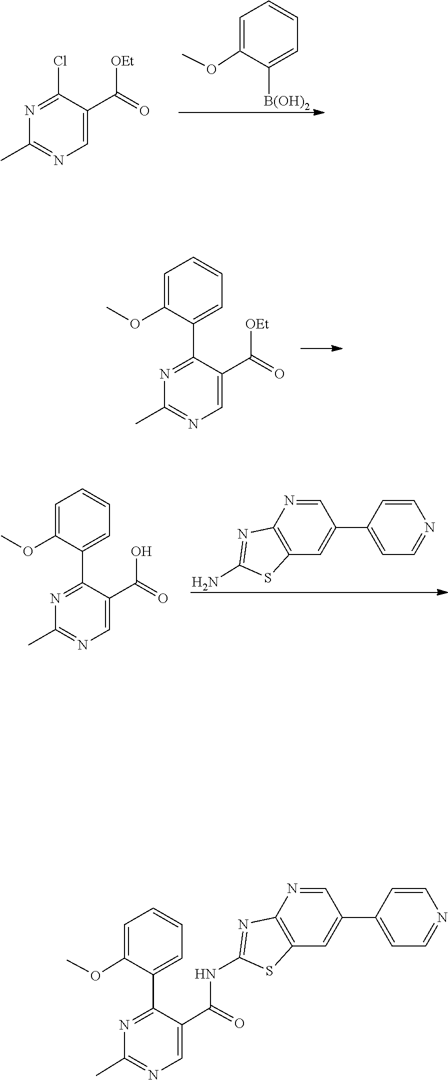 [SMILES: CCOC(=O)c1cnc(C)nc1Cl ; COc1ccccc1B(O)O ; CCOC(=O)c1cnc(C)nc1-c1ccccc1OC ; COc1ccccc1-c1nc(C)ncc1C(=O)O ; Nc1nc2ncc(-c3ccncc3)cc2s1 ; COc1ccccc1-c1nc(C)ncc1C(=O)Nc1nc2ncc(-c3ccncc3)cc2s1]

Step 1: To a solution of ethyl 4-chloro-2-methylpyrimidine-5-carboxylate (100 mg, 0.498 mmol) in DME (0.5 mL) and water (1 mL) was added (2-methoxyphenyl)boronic acid (75.7 mg, 0.498 mmol), Pd(PPh₃)₄ (17.3 mg, 0.015 mmol) and Na₂CO₃ (132 mg, 1.25 mmol) and the mixture was heated at 100° C. overnight under N2. The mixture was diluted with water (40 mL), extracted with EtOAc (40 mL×3) and the combined organic layers washed with brine, dried over Na₂SO₄, filtered and concentrated under reduced pressure. The mixture was purified by prep-TLC (Pet. ether/EtOAc=5/1, v/v) to afford ethyl 4-(2-methoxyphenyl)-2-methylpyrimidine-5-carboxylate (80 mg, 10%) as a yellow solid. LCMS (ESI, m/z): 273 [M+H]⁺, ¹H NMR (400 MHz, DMSO-d₆) δ 8.92 (s, 1H), 7.54-7.46 (m, 2H), 7.12-7.05 (m, 2H), 4.11 (q, J=7.2 Hz, 2H), 3.67 (s, 3H), 2.71 (s, 3H), 1.04 (t, J=7.2 Hz, 3H).

Step 2: To a solution of ethyl 4-(2-methoxyphenyl)-2-methylpyrimidine-5-carboxylate (80 mg, 0.294 mmol) in MeOH (1 mL) and water (1 mL) was added NaOH (35.2 mg, 0.881 mmol) and the mixture was heated at 50° C. for 3 h. The mixture was adjusted to pH 4-5 with 2 M aqueous HCl and extracted with EtOAc (40 mL×3). The combined organic layers were washed with brine, dried over Na₂SO₄, filtered and concentrated under reduced pressure to afford 4-(2-methoxyphenyl)-2-methylpyrimidine-5-carboxylic acid (50 mg, 69%) as a white solid. LCMS m/z=245 [M+H]⁺, ¹H NMR (400 MHz, DMSO-d₆) δ 8.91 (s, 1H), 7.51-7.43 (m, 2H), 7.07 (t, J=7.8 Hz, 2H), 3.69 (s, 3H), 2.69 (s, 3H).

Step 3: To a solution of 4-(2-methoxyphenyl)-2-methylpyrimidine-5-carboxylic acid (150 mg, 0.614 mmol) in NMP (4 mL) was added HATU (350 mg, 0.921 mmol) and DIPEA (238 mg, 1.84 mmol) and the mixture was stirred at room temperature for 2 h. 6-(Pyridin-4-yl)thiazolo[4,5-b]pyridin-2-amine (168 mg, 0.737 mmol) was added and the mixture was heated at 80° C. overnight. The mixture was diluted with water (40 mL), extracted with EtOAc (40 mL×3) and the combined organic layers washed with brine, dried over Na₂SO₄, filtered and concentrated under reduced pressure. The residue was purified by prep-TLC (DCM/MeOH=20/1, v/v) to afford 4-(2-methoxyphenyl)-2-methyl-N-(6-(pyridin-4-yl)thiazolo[4,5-b]pyridin-2-yl)pyrimidine-5-carboxamide (129 mg, 46%) as a white solid. LCMS (ESI, m/z): 455 [M+H]⁺, ¹H NMR (400 MHz, DMSO-d₆) δ 13.3 (s, 1H), 9.02 (d, J=2.2 Hz, 1H), 8.99 (s, 1H), 8.93 (d, J=2.2 Hz, 1H), 8.72-8.68 (m, 2H), 7.86-7.82 (m, 2H), 7.69 (dd, J=7.6, 1.8 Hz, 1H), 7.49 (td, J=7.8, 1.8 Hz, 1H), 7.16 (t, J=7.4 Hz, 1H), 7.01 (d, J=8.2 Hz, 1H), 3.51 (s, 3H), 2.76 (s, 3H).

### Example 205: Synthesis of tert-Butyl 4-(2-cyano-5-((6-(pyridin-4-yl)thiazolo[4,5-b]pyridin-2-yl)carbamoyl)pyridin-4-yl)piperazine-1-carboxylate

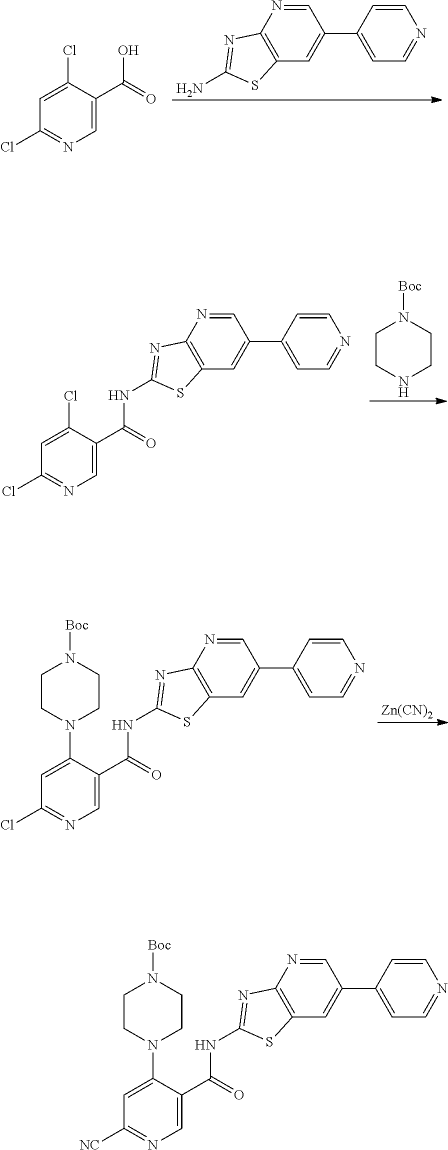

Step 1: To a solution of 4,6-dichloronicotinic acid (200 mg, 1.04 mmol) in THF (5 mL) was added SOCl₂ (248 mg, 2.08 mmol) and DMF (catalytic) and the mixture was heated at 60° C. for 1 h. The mixture was concentrated under reduced pressure and the residue was treated with a solution of 6-(pyridin-4-yl)thiazolo[4,5-b]pyridin-2-amine (285 mg, 1.25 mmol) and TEA (316 mg, 3.13 mmol) in THF (5 mL) then stirred at room temperature overnight. The mixture was concentrated under reduced pressure, diluted with water (50 mL) and extracted with EtOAc (2×50 mL). The combined organic layers were washed with water (50 mL), brine (50 mL), dried over Na₂SO₄, filtered and concentrated under reduced pressure. The residue was purified by prep-TLC (DCM/MeOH=30/1, v/v) to give 4,6-dichloro-N-(6-(pyridin-4-yl)thiazolo[4,5-b]pyridin-2-yl)nicotinamide (80 mg, 16%) as an off-white solid. LCMS (ESI, m/z): 402 [M+H]⁺, ¹H NMR (400 MHz, DMSO-d₆): δ 13.6 (s, 1H), 9.05 (d, J=2.2 Hz, 1H), 9.00 (d, J=2.2 Hz, 1H), 8.84 (s, 1H), 8.70 (d, J=6.2 Hz, 2H), 8.06 (s, 1H), 7.86 (d, J=6.2 Hz, 2H).

Step 2: To a solution of 4,6-dichloro-N-(6-(pyridin-4-yl)thiazolo[4,5-b]pyridin-2-yl)nicotinamide (250 mg, 0.622 mmol) and tert-butyl piperazine-1-carboxylate (116 mg, 0.622 mmol) in NMP (3 mL) was added DIPEA (402 mg, 3.11 mmol) and the mixture was heated at 85° C. overnight. The mixture was diluted with water (50 mL), extracted with EtOAc (2×50 mL) and the combined organic layers washed with water (50 mL), brine (50 mL), dried over Na₂SO₄, filtered and concentrated under reduced pressure. The residue was purified by prep-TLC (DCM/MeOH=20/1, v/v) to give tert-butyl4-(2-chloro-5-((6-(pyridin-4-yl)thiazolo[4,5-b]pyridin-2-yl)carbamoyl)pyridin-4-yl)piperazine-1-carboxylate (150 mg, 50%) as a yellow solid. LCMS (ESI, m/z): 552 [M+H], ¹H NMR (400 MHz, DMSO-d₆): δ 13.3 (s, 1H), 9.03 (s, 1H), 8.97 (s, 1H), 8.70 (s, 2H), 8.36 (s, 1H), 7.85 (s, 2H), 7.09 (s, 1H), 3.43 (s, 4H), 3.24 (s, 4H), 1.38 (s, 9H).

Step 3: To a solution of tert-butyl4-(2-chloro-5-((6-(pyridin-4-yl)thiazolo[4,5-b]pyridin-2-yl)carbamoyl)pyridin-4-yl)piperazine-1-carboxylate (75 mg, 0.14 mmol) and Zn(CN)₂ (21 mg, 0.18 mmol) in DMA (2 mL) was added [PdCl(allyl)]2 (25 mg, 0.068 mmol) and X-Phos (10 mg, 0.02 mmol) and the mixture was heated at 120° C. overnight under N2. The mixture was diluted with water (50 mL), extracted with EtOAc (2×50 mL) and the combined organic layers washed with water (50 mL), brine (50 mL), dried over Na₂SO₄, filtered and concentrated under reduced pressure. The residue was purified by prep-TLC (DCM/MeOH=15/1, v/v) to give tert-butyl4-(2-cyano-5-((6-(pyridin-4-yl)thiazolo[4,5-b]pyridin-2-yl)carbamoyl)pyridin-4-yl)piperazine-1-carboxylate (4 mg, 6%) as a yellow solid. LCMS (ESI, m/z): 543 [M+H]⁺, ¹H NMR (400 MHz, DMSO-d₆): δ 13.4 (s, 1H), 8.99 (d, J=2.2 Hz, 1H), 8.89 (s, 1H), 8.74-8.63 (m, 2H), 8.56 (s, 1H), 7.88-7.81 (m, 2H), 7.67 (s, 1H), 3.47-3.41 (m, 4H), 3.34-3.28 (m, 4H, partially obscured by water peak) 1.38 (s, 9H).

### Example 206: Synthesis of 4-(5-Cyano-2-methoxyphenyl)-6-methyl-N-(6-(2-methylpyridin-4-yl)thiazolo[4,5-b]pyridin-2-yl)nicotinamide

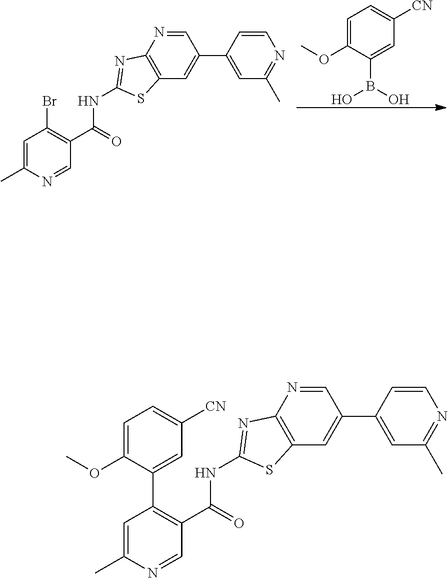

To a solution of 4-bromo-6-methyl-N-(6-(2-methylpyridin-4-yl)thiazolo[4,5-b]pyridin-2-yl)nicotinamide (40 mg, 0.091 mmol), (5-cyano-2-methoxyphenyl)boronic acid (16 mg, 0.091 mmol) and Na₂CO₃ (28.9 mg, 0.273 mmol) in 1,4-dioxane (2 mL) and water (0.4 mL) was added Pd(dppf)Cl₂ (6.59 mg, 0.009 mmol) and the mixture was heated at 100° C. overnight under N2. The mixture was diluted with water (10 mL), extracted with EtOAc (10 mL×3) and the combined organic phases dried over Na₂SO₄, filtered and concentrated under reduced pressure. The residue was purified by prep-TLC (DCM/MeOH=30/1, v/v) to afford 4-(5-cyano-2-methoxyphenyl)-6-methyl-N-(6-(2-methylpyridin-4-yl)thiazolo[4,5-b]pyridin-2-yl)nicotinamide (10 mg, 22%) as a yellow solid. LCMS (ESI, m/z): 493 [M+H]⁺, ¹H NMR (400 MHz, DMSO-d₆): δ 13.2 (s, 1H), 8.98 (d, J=2.2 Hz, 1H), 8.87 (d, J=2.2 Hz, 1H), 8.82 (s, 1H), 8.55 (d, J=5.2 Hz, 1H), 7.94-7.87 (m, 2H), 7.70 (d, J=1.6 Hz, 1H), 7.64-7.58 (m, 1H), 7.44 (s, 1H), 7.17 (d, J=9.2 Hz, 1H), 3.58 (s, 3H), 2.61 (s, 3H), 2.56 (s, 3H).

### Example 207: Synthesis of N-(6-(5-(Hydroxymethyl)pyridin-2-yl)thiazolo[4,5-b]pyrazin-2-yl)-4-(2-methoxyphenyl)-6-methylnicotinamide

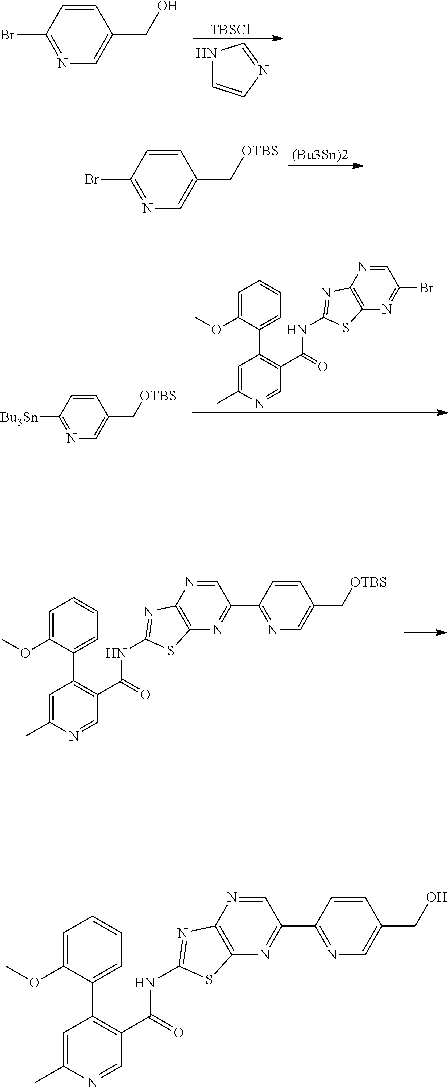

Step 1: To a solution of (6-bromopyridin-3-yl)methanol (2 g, 10 mmol) in DCM (20 mL) was added TBSCl (2.0 g, 14 mmol) and 1H-imidazole (1.1 g, 16 mmol) and the mixture was stirred at room temperature for 2 h. The mixture was diluted with water (40 mL), extracted with EtOAc (60 mL×3) and the combined organic layers washed with brine, dried over Na₂SO₄, filtered and concentrated under reduced pressure to afford 2-bromo-5-(((tert-butyldimethylsilyl)oxy)methyl)pyridine (3.3 g, 99%) as a yellow oil. LCMS (ESI, m/z): 302, 304 [M+H], ¹H NMR (400 MHz, DMSO-d₆): δ 8.34-8.30 (m, 1H), 7.67 (dd, J=8.2, 2.3 Hz, 1H), 7.62 (d, J=8.0 Hz, 1H) 4.72 (s, 2H), 0.89 (s, 9H), 0.08 (s, 6H).

Step 2: To a solution of 1,1,1,2,2,2-hexabutyldistannane (1.15 g, 1.98 mmol) and Pd(PPh₃)₄ (114 mg, 0.09 mmol) in 1,4-dioxane (9 mL) was added 2-bromo-5-(((tert-butyldimethylsilyl)oxy)methyl)pyridine (300 mg, 0.99 mmol) and the mixture was heated at 110° C. overnight under N2. The mixture was diluted with a 5% aqueous potassium fluoride solution (30 mL) and extracted with EtOAc (60 mL×3). The combined organic layers were washed with brine (20 mL), dried over Na₂SO₄, filtered and concentrated under reduced pressure to give 5-(((tert-butyldimethylsilyl)oxy)methyl)-2-(tributylstannyl)pyridine (500 mg, 98%) as a yellow oil, which was used without further purification. LCMS (ESI, m/z): 514 [M+H]⁺.

Step 3: To a solution of 5-(((tert-butyldimethylsilyl)oxy)methyl)-2-(tributylstannyl)pyridine (500 mg, 0.97 mmol) and Pd(PPh₃)₄ (43 mg, 0.03 mmol) in 1,4-dioxane (9 mL) was added N-(6-bromothiazolo[4,5-b]pyrazin-2-yl)-4-(2-methoxyphenyl)-6-methylnicotinamide (170 mg, 0.37 mmol) and the mixture was heated at 110° C. overnight under N2. The mixture was diluted with a 5% aqueous potassium fluoride solution (20 mL) and extracted with EtOAc (50 mL×3). The combined organic layers were washed with brine (20 mL), dried over Na₂SO₄, filtered and concentrated under reduced pressure. The residue was purified by prep-TLC (DCM/MeOH=20/1, v/v) to give N-(6-(5-(((tert-butyldimethylsilyl)oxy)methyl)pyridin-2-yl)thiazolo[4,5-b]pyrazin-2-yl)-4-(2-methoxyphenyl)-6-methylnicotinamide (100 mg, 45%) as a yellow solid. LCMS (ESI, m/z): 599 [M+H], ¹H NMR (400 MHz, DMSO-d₆): δ 13.4 (s, 1H), 9.49 (s, 1H), 8.79 (s, 1H), 8.67 (s, 1H), 8.35 (d, J=8.2 Hz, 1H), 7.90 (d, J=7.8 Hz, 1H), 7.39 (d, J=7.2 Hz, 2H), 7.32 (s, 1H), 7.10 (t, J=7.6 Hz, 1H), 7.00-6.96 (m, 1H), 4.84 (s, 2H), 3.52 (s, 3H), 2.59 (s, 3H), 0.92 (s, 9H), 0.12 (s, 6H).

Step 4: A solution of N-(6-(5-(((tert-butyldimethylsilyl)oxy)methyl)pyridin-2-yl)thiazolo[4,5-b]pyrazin-2-yl)-4-(2-methoxyphenyl)-6-methylnicotinamide (50 mg, 0.08 mmol) and TBAF (65 mg, 0.25 mmol) in THF (5 mL) was stirred at room temperature overnight. The mixture was diluted with water (10 mL), extracted with EtOAc (30 mL×3) and the combined organic layers washed with brine (20 mL), dried over Na₂SO₄, filtered and concentrated under reduced pressure. The residue was rinsed with a dilute citric acid aqueous solution (10 mL) and triturated with MeOH (10 mL) to give N-(6-(5-(hydroxymethyl)pyridin-2-yl)thiazolo[4,5-b]pyrazin-2-yl)-4-(2-methoxyphenyl)-6-methylnicotinamide (20 mg, 41%) as a yellow solid. LCMS (ESI, m/z): 485 [M+H], ¹H NMR (400 MHz, DMSO-d₆): δ 13.5 (s, 1H), 9.55 (s, 1H), 8.86 (s, 1H), 8.68 (d, J=2.0 Hz, 1H), 8.37 (d, J=8.2 Hz, 1H), 7.95 (dd, J=8.2, 2.2 Hz, 1H), 7.51 (s, 1H), 7.47-7.41 (m, 2H), 7.14 (t, J=7.6 Hz, 1H), 7.01 (d, J=8.4 Hz, 1H), 4.63 (s, 2H), 3.53 (s, 3H), 2.65 (s, 3H).

### Example 208: Synthesis of N-(6-(5-(Hydroxymethyl)pyridin-2-yl)thiazolo[4,5-b]pyridin-2-yl)-4-(2-methoxyphenyl)-6-methylnicotinamide

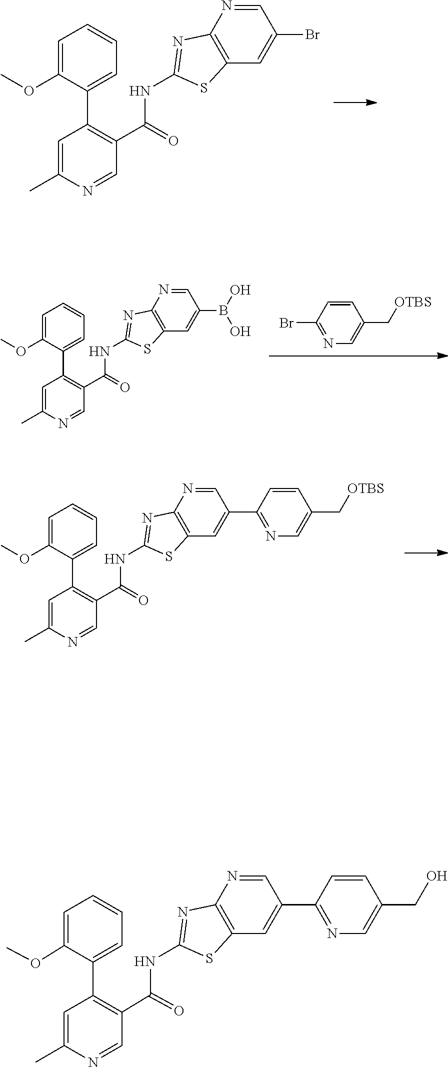 [SMILES: COc1ccccc1-c1cc(C)ncc1C(=O)Nc1nc2ncc(Br)cc2s1 ; COc1ccccc1-c1cc(C)ncc1C(=O)Nc1nc2ncc(B(O)O)cc2s1 ; CC(C)(C)[Si](C)(C)OCc1ccc(Br)nc1 ; COc1ccccc1-c1cc(C)ncc1C(=O)Nc1nc2ncc(-c3ccc(CO[Si](C)(C)C(C)(C)C)cn3)cc2s1 ; COc1ccccc1-c1cc(C)ncc1C(=O)Nc1nc2ncc(-c3ccc(CO)cn3)cc2s1]

Step 1: To a solution of N-(6-bromothiazolo[4,5-b]pyridin-2-yl)-4-(2-methoxyphenyl)-6-methylnicotinamide (300 mg, 0.66 mmol) in 1,4-dioxane (6 mL) was added Pin₂B₂ (502 mg, 0.98 mmol), Pd(dppf)Cl₂ (48 mg, 0.066 mmol) and KOAc (194 mg, 1.98 mmol) and the mixture was heated at 100° C. overnight under a N2 atmosphere. The mixture was filtered and the filtrate concentrated under reduced pressure. The residue was purified by silica gel chromatography (DCM/MeOH=10/1) to give (2-(4-(2-methoxyphenyl)-6-methylnicotinamido)thiazolo[4,5-b]pyridin-6-yl)boronic acid (150 mg, 54%) as a yellow solid. LCMS m/z=421 [M+H]⁺.

Step 2: To a solution of 2-bromo-5-(((tert-butyldimethylsilyl)oxy)methyl)pyridine (100 mg, 0.330 mmol) in 1,4-dioxane (1 mL) and water (0.3 mL) was added (2-(4-(2-methoxyphenyl)-6-methylnicotinamido)thiazolo[4,5-b]pyridin-6-yl)boronic acid (139 mg, 0.330 mmol), Pd(dppf)Cl₂ (24.0 mg, 0.033 mmol) and Na₂CO₃ (82.4 mg, 0.992 mmol) and the mixture was heated at 100° C. overnight under a N2 atmosphere. The mixture was diluted with water (40 mL), extracted with EtOAc (60 mL×3) and the combined organic layers washed with brine, dried over Na₂SO₄, filtered and concentrated under reduced pressure. The residue was purified by prep-TLC (DCM/MeOH=10/1, v/v) to afford N-(6-(5-(((tert-butyldimethylsilyl)oxy)methyl)pyridin-2-yl)thiazolo[4,5-b]pyridin-2-yl)-4-(2-methoxyphenyl)-6-methylnicotinamide (50 mg, 25%) as a yellow solid. LCMS (ESI, m/z): 598 [M+H]⁺, ¹H NMR (400 MHz, DMSO-d₆): δ 13.1 (s, 1H), 9.25 (d, J=2.2 Hz, 1H), 9.07 (d, J=2.2 Hz, 1H), 8.74 (s, 1H), 8.65 (d, J=2.2 Hz, 1H), 8.07 (d, J=8.2 Hz, 1H), 7.84 (dd, J=8.2, 2.2 Hz, 1H), 7.39 (td, J=7.2, 1.8 Hz, 2H), 7.33 (s, 1H), 7.10 (t, J=7.4 Hz, 1H), 6.97 (d, J=8.4 Hz, 1H), 4.82 (s, 2H), 3.50 (s, 3H), 2.59 (s, 3H), 0.92 (s, 9H), 0.12 (s, 6H).

Step 3: To a solution of N-(6-(5-(((tert-butyldimethylsilyl)oxy)methyl)pyridin-2-yl)thiazolo[4,5-b]pyridin-2-yl)-4-(2-methoxyphenyl)-6-methylnicotinamide (40 mg, 0.14 mmol) in THF (1 mL) was added TBAF (0.2 mL, 0.222 mmol) and the mixture was stirred at room temperature overnight. The mixture was diluted with water (20 mL), extracted with EtOAc (20 mL×3) and the combined organic layers washed with brine, dried over Na₂SO₄, filtered and concentrated under reduced pressure. The residue was purified by prep-TLC (DCM/MeOH=15/1, v/v) to afford N-(6-(5-(hydroxymethyl)pyridin-2-yl)thiazolo[4,5-b]pyridin-2-yl)-4-(2-methoxyphenyl)-6-methylnicotinamide (20 mg, 44%) as a white solid. LCMS (ESI, m/z): 484 [M+H]⁺, ¹H NMR (400 MHz, DMSO-d₆): δ 13.1 (s, 1H), 9.25 (d, J=2.2 Hz, 1H), 9.08-9.04 (m, 1H), 8.75 (s, 1H), 8.66-8.62 (m, 1H), 8.05 (d, J=8.2 Hz, 1H), 7.88-7.83 (m, 1H), 7.43-7.37 (m, 2H), 7.33 (s, 1H), 7.10 (t, J=7.4 Hz, 1H), 6.98 (d, J=8.6 Hz, 1H), 5.39 (t, J=5.8 Hz, 1H), 4.59 (d, J=5.6 Hz, 2H), 3.50 (s, 3H), 2.59 (s, 3H).

### Example 209: Synthesis of (Cis and Trans)-N-(6-(4-Hydroxycyclohexyl)thiazolo[4,5-b]pyrazin-2-yl)-4-(2-methoxyphenyl)-6-methylnicotinamide

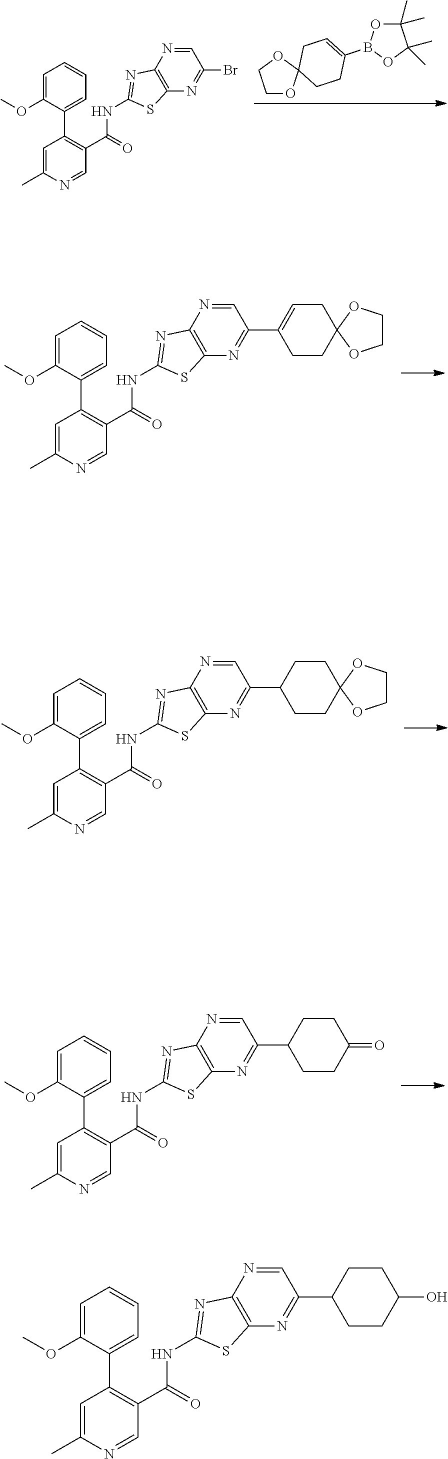

Step 1: To a solution of N-(6-bromothiazolo[4,5-b]pyrazin-2-yl)-4-(2-methoxyphenyl)-6-methylnicotinamide (284 mg, 0.622 mmol) in 1,4-dioxane (4 mL) and water (1 mL) was added 4,4,5,5-tetramethyl-2-(1,4-dioxaspiro[4.5]dec-7-en-8-yl)-1,3,2-dioxaborolane (248 mg, 0.933 mmol), Pd(PPh₃)₄ (71.9 mg, 0.062 mmol) and Na₂CO₃ (198 mg, 1.87 mmol) and the mixture was heated at 100° C. overnight under N2. The mixture was diluted with water (40 mL), extracted with EtOAc (60 mL×3) and the combined organic layers washed with brine, dried over Na₂SO₄, filtered and concentrated under reduced pressure. The residue was purified by prep-TLC (DCM/MeOH=20/1, v/v) to afford N-(6-(1,4-dioxaspiro[4.5]dec-7-en-8-yl)thiazolo[4,5-b]pyrazin-2-yl)-4-(2-methoxyphenyl)-6-methylnicotinamide (230 mg, 71%) as a yellow solid. LCMS (ESI, m/z): 516 [M+H]⁺, ¹H NMR (400 MHz, DMSO-d₆): δ 13.2 (s, 1H), 8.83 (s, 1H), 8.73 (s, 1H), 7.40 (ddd, J=7.6, 6.0, 1.8 Hz, 2H), 7.34 (s, 1H), 7.10 (t, J=7.6 Hz, 1H), 6.98 (d, J=8.6 Hz, 1H), 6.72-6.68 (m, 1H), 3.93 (s, 4H), 3.51 (s, 3H), 2.72 (s, 2H), 2.59 (s, 3H), 2.46 (s, 2H), 1.84 (t, J=6.6 Hz, 2H).

Step 2: To a solution of N-(6-(1,4-dioxaspiro[4.5]dec-7-en-8-yl)thiazolo[4,5-b]pyrazin-2-yl)-4-(2-methoxyphenyl)-6-methylnicotinamide (100 mg, 0.194 mmol) in MeOH (1 mL) was added 10% Pd/C (100 mg), 20% Pd(OH)₂/C (50 mg) and ammonium formate (122 mg, 1.94 mmol) and the mixture was heated at 50° C. for 24 h. A further aliquot of 10% Pd/C (100 mg) and 20% Pd(OH)₂/C (50 mg) was added and heating was continued at 50° C. for 24 h. The mixture was filtered and the filtrate concentrated under reduced pressure. The residue was purified by prep-TLC (DCM/MeOH=15/1, v/v) to afford N-(6-(1,4-dioxaspiro[4.5]decan-8-yl)thiazolo[4,5-b]pyrazin-2-yl)-4-(2-methoxyphenyl)-6-methylnicotinamide (30 mg, 30%) as a yellow solid. LCMS (ESI, m/z): 518 [M+H].

Step 3: To a solution of N-(6-(1,4-dioxaspiro[4.5]decan-8-yl)thiazolo[4,5-b]pyrazin-2-yl)-4-(2-methoxyphenyl)-6-methylnicotinamide (30 mg, 0.058 mmol) in ACN (1 mL) and water (0.4 mL) was added a 3 M aqueous HCl solution (0.096 mL, 0.29 mmol) and the mixture was stirred at room temperature for 30 min. The mixture was diluted with water (10 mL), extracted with EtOAc (20 mL×3) and the combined organic layers washed with brine, dried over Na₂SO₄, filtered and concentrated under reduced pressure. The residue was purified by prep-TLC (DCM/MeOH=20/1, v/v) to afford 4-(2-methoxyphenyl)-6-methyl-N-(6-(4-oxocyclohexyl)thiazolo[4,5-b]pyrazin-2-yl)nicotinamide (20 mg, 58%) as a yellow solid. LCMS (ESI, m/z): 474 [M+H]⁺.

Step 4: To a solution of 4-(2-methoxyphenyl)-6-methyl-N-(6-(4-oxocyclohexyl)thiazolo[4,5-b]pyrazin-2-yl)nicotinamide (20 mg, 0.042 mmol) in EtOH (1.5 mL) was added NaBH₄ (2.40 mg, 0.634 mmol) and the mixture was stirred at room temperature for 30 min. The mixture was diluted with water (10 mL), extracted with EtOAc (10 mL×3) and the combined organic layers washed with brine, dried over Na₂SO₄, filtered and concentrated under reduced pressure. The residue was purified by prep-TLC (DCM/MeOH=15/1, v/v) to afford N-(6-(4-hydroxycyclohexyl)thiazolo[4,5-b]pyrazin-2-yl)-4-(2-methoxyphenyl)-6-methylnicotinamide (10 mg, 50%) as a white solid. LCMS (ESI, m/z): 476 [M+H]⁺, ¹H NMR (400 MHz, DMSO-d₆): δ 13.1 (s, 1H), 8.74 (s, 1H), 8.51 (s, 1H), 7.42-7.36 (m, 2H), 7.31 (s, 1H), 7.09 (t, J=7.6 Hz, 1H), 6.97 (d, J=8.6 Hz, 1H), 4.60 (d, J=4.4 Hz, 0.85H), 4.38 (d, J=3.2 Hz, 0.15H), 3.92-3.88 (m, 0.15H) 3.50 (s, 3H), 3.50-3.42 (m, 0.85H), 2.86-2.74 (m, 1H), 2.58 (s, 3H), 2.03-1.82 (m, 4H), 1.71-1.55 (m, 2H), 1.37-1.24 (m, 2H).

### Example 210A & 210B: Synthesis of N-(6-((cis)-4-Hydroxycyclohexyl)thiazolo[4,5-b]pyrazin-2-yl)-4-(2-methoxyphenyl)-6-methylnicotinamide (210A) and N-(6-((trans)-4-Hydroxycyclohexyl)thiazolo[4,5-b]pyrazin-2-yl)-4-(2-methoxyphenyl)-6-methylnicotinamide (210B)

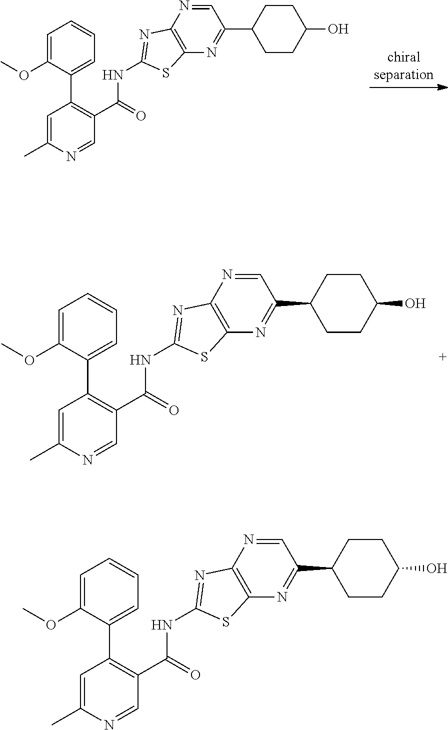 [SMILES: COc1ccccc1-c1cc(C)ncc1C(=O)Nc1nc2ncc(C3CCC(O)CC3)nc2s1 ; COc1ccccc1-c1cc(C)ncc1C(=O)Nc1nc2ncc([C@H]3CC[C@@H](O)CC3)nc2s1 ; COc1ccccc1-c1cc(C)ncc1C(=O)Nc1nc2ncc([C@H]3CC[C@H](O)CC3)nc2s1]

N-(6-(4-Hydroxycyclohexyl)thiazolo[4,5-b]pyrazin-2-yl)-4-(2-methoxyphenyl)-6-methylnicotinamide (Example 209) was purified by chiral SFC (Column: Daicel Chiralpak, AD-H, 30×250 mm; Mobile Phase: 10% Ethanol/hexane, 90% carbon dioxide; Flow rate: 60 mL/min) to afford two peaks:

N-(6-((cis)-4-Hydroxycyclohexyl)thiazolo[4,5-b]pyrazin-2-yl)-4-(2-methoxyphenyl)-6-methylnicotinamide: LCMS (ESI, m/z): 476 [M+H]⁺, ¹H NMR (400 MHz, DMSO-d₆): δ 13.2 (s, 1H), 8.75 (s, 1H), 8.55-8.48 (m, 1H), 7.44-7.36 (m, 2H), 7.35-7.30 (m, 1H), 7.09 (t, J=7.6 Hz, 1H), 6.98 (d, J=8.6 Hz, 1H), 4.38 (d, J=3.2 Hz, 1H), 3.93-3.87 (m, 1H), 3.51 (s, 3H), 2.92-2.80 (m, 1H), 2.59 (s, 3H), 2.08-1.95 (m, 2H), 1.81-1.71 (m, 2H), 1.66-1.53 (m, 4H).

N-(6-((trans)-4-Hydroxycyclohexyl)thiazolo[4,5-b]pyrazin-2-yl)-4-(2-methoxyphenyl)-6-methylnicotinamide: LCMS (ESI, m/z): 476 [M+H]; ¹H NMR (400 MHz, DMSO-d₆): δ 13.2 (s, 1H), 8.74 (s, 1H), 8.54 (s, 1H), 7.43-7.37 (m, 2H), 7.33 (s, 1H), 7.10 (t, J=7.4 Hz, 1H), 6.97 (d, J=8.6 Hz, 1H), 4.60 (d, J=4.4 Hz, 1H), 3.50 (s, 3H), 3.50-3.43 (m, 1H), 2.86-2.76 (m, 1H), 2.59 (s, 3H), 1.99-1.84 (m, 4H), 1.70-1.59 (m, 2H), 1.37-1.26 (m, 2H).

### Example 211: Synthesis of (Racemic)-4-(2-Methoxyphenyl)-6-methyl-N-(6-(tetrahydrofuran-2-yl)thiazolo[4,5-b]pyridin-2-yl)nicotinamide

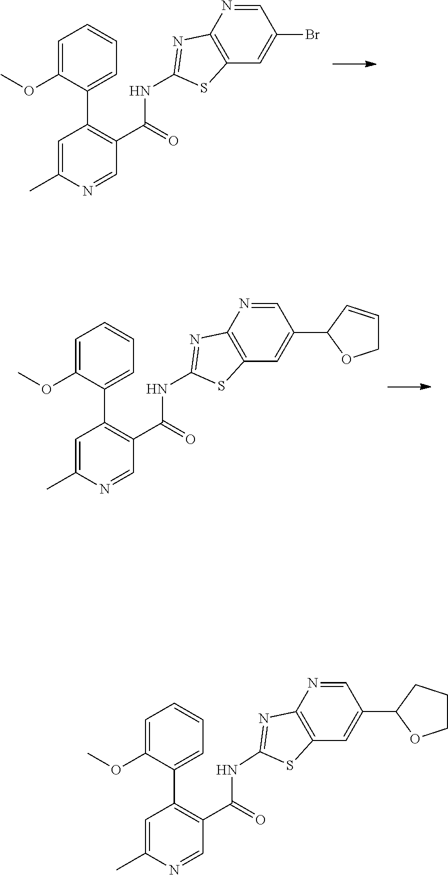 [SMILES: COc1ccccc1-c1cc(C)ncc1C(=O)Nc1nc2ncc(Br)cc2s1 ; COc1ccccc1-c1cc(C)ncc1C(=O)Nc1nc2ncc(C3C=CCO3)cc2s1 ; COc1ccccc1-c1cc(C)ncc1C(=O)Nc1nc2ncc(C3CCCO3)cc2s1]

Step 1: A solution of N-(6-bromothiazolo[4,5-b]pyridin-2-yl)-4-(2-methoxyphenyl)-6-methylnicotinamide (50 mg, 0.11 mmol), 2,3-dihydrofuran (15 mg, 0.22 mmol), N-cyclohexyl-N-methylcyclohexanamine (43 mg, 0.22 mmol), [(t-Bu)₃Ph]BF₄ (3 mg, 0.011 mmol) and Pd₂(dba)₃ (10 mg, 0.011 mmol) in 1,4-dioxane (1 mL) was heated at 80° C. for 2 h under N2. The mixture was poured into water (20 mL), extracted with EtOAc (20 mL×3) and the combined organic layers washed with brine (20 mL), dried over Na₂SO₄, filtered and concentrated under reduced pressure to afford N-(6-(2,5-dihydrofuran-2-yl)thiazolo[4,5-b]pyridin-2-yl)-4-(2-methoxyphenyl)-6-methylnicotinamide (60 mg, crude) as a brown solid. LCMS (ESI, m/z): 445 [M+H]+

Step 2: A mixture of N-(6-(2,5-dihydrofuran-2-yl)thiazolo[4,5-b]pyridin-2-yl)-4-(2-methoxyphenyl)-6-methylnicotinamide (40 mg, 0.089 mmol) and 10% Pd/C (10 mg) in MeOH (3 mL) was stirred at room temperature under a H₂ atmosphere (1 atm) overnight. The mixture was filtered and the filtrate concentrated under reduced pressure. The residue was purified by prep-HPLC to afford (Racemic)-4-(2-methoxyphenyl)-6-methyl-N-(6-(tetrahydrofuran-2-yl)thiazolo[4,5-b]pyridin-2-yl)nicotinamide (20 mg, 25%) as a white solid. LCMS (ESI, m/z): 447 [M+H]⁺, ¹H NMR (400 MHz, DMSO-d₆): δ 12.9 (s, 1H), 8.72 (s, 1H), 8.50 (d, J=2.2 Hz, 1H), 8.37 (d, J=2.1 Hz, 1H), 7.42-7.36 (m, 2H), 7.33 (s, 1H), 7.09 (t, J=7.4 Hz, 1H), 6.96 (d, J=8.6 Hz, 1H), 4.95 (t, J=7.2 Hz, 1H), 4.06-4.00 (m, 1H), 3.88-3.80 (m, 1H), 3.48 (s, 3H), 2.58 (s, 3H), 2.39-2.32 (m, 1H), 2.04-1.94 (m, 2H), 1.82-1.70 (m, 1H).

### Example 212: Synthesis of (Racemic)-N-(6-((1,4-Dioxan-2-yl)methyl)thiazolo[4,5-b]pyrazin-2-yl)-4-(2-methoxyphenyl)-6-methylnicotinamide

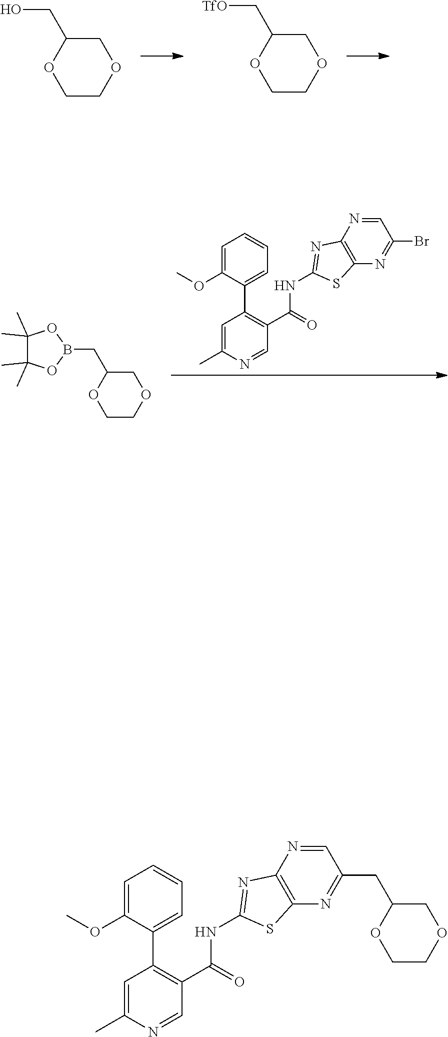 [SMILES: OCC1COCCO1 ; O=S(=O)(OCC1COCCO1)C(F)(F)F ; CC1(C)OB(CC2COCCO2)OC1(C)C ; COc1ccccc1-c1cc(C)ncc1C(=O)Nc1nc2ncc(Br)nc2s1 ; COc1ccccc1-c1cc(C)ncc1C(=O)Nc1nc2ncc(CC3COCCO3)nc2s1]

Step 1: To a solution of (1,4-dioxan-2-yl)methanol (100 mg, 0.84 mmol) in DCM (3 mL) at 0° C. was added Tf₂O (250 mg, 0.88 mmol) and pyridine (3 drops) and the mixture was stirred at room temperature for 1 h. The mixture was diluted with water (20 mL), extracted with EtOAc (2×20 mL) and the combined organic phases washed with water (20 mL), brine (20 mL), dried over Na₂SO₄, filtered and concentrated under reduced pressure to give (1,4-dioxan-2-yl)methyl trifluoromethanesulfonate (120 mg, 56%) as colorless oil. ¹H NMR (400 MHz, DMSO-d₆): δ 4.38-4.24 (m, 2H), 3.87-3.76 (m, 2H), 3.75-3.62 (m, 3H), 3.55-3.46 (m, 1H), 3.34-3.29 (m, 1H).

Step 2: To a solution (1,4-dioxan-2-yl)methyl trifluoromethanesulfonate (120 mg, 0.47 mmol) in DMF (3 mL) was added Pin₂B₂ (183 mg, 0.71 mmol), PPh₃ resin (17 mg, 0.062 mmol), CuI (10 mg, 0.047 mmol) and LiOMe (36 mg, 0.95 mmol) and the mixture was stirred at room temperature overnight. The mixture was diluted with water (20 mL), extracted with EtOAc (2×20 mL) and the combined organic phases washed with water (20 mL), brine (20 mL), dried over Na₂SO₄, filtered and concentrated under reduced pressure to give 2-((1,4-dioxan-2-yl)methyl)-4,4,5,5-tetramethyl-1,3,2-dioxaborolane (100 mg, 91%) as colorless oil. ¹H NMR (400 MHz, DMSO-d₆): δ 3.92 (d, J=3.0 Hz, 2H), 3.69-3.51 (m, 3H), 3.46-3.36 (m, 1H), 3.14-3.04 (m, 1H), 2.89 (d, J=3.2 Hz, 1H), 2.73 (d, J=3.2 Hz, 1H), 1.09-1.05 (m, 12H).

Step 3: To a solution of N-(6-bromothiazolo[4,5-b]pyrazin-2-yl)-4-(2-methoxyphenyl)-6-methylnicotinamide (70 mg, 0.15 mmol) in 1,4-dioxane (2 mL) and water (0.5 mL) was added 2-((1,4-dioxan-2-yl)methyl)-4,4,5,5-tetramethyl-1,3,2-dioxaborolane (52 mg, 0.23 mmol), Pd(OAc)₂ (4 mg, 0.015 mmol), Butyldi-1-adamantylphosphine (11 mg, 0.03 mmol) and K₂CO₃ (42 mg, 0.3 mmol) and the mixture was heated at 100° C. overnight under N2. The mixture was diluted with water (20 mL), extracted with EtOAc (2×20 mL) and the combined organic phases washed with water (20 mL), brine (20 mL), dried over Na₂SO₄, filtered and concentrated under reduced pressure. The residue was purified by prep-TLC (DCM/MeOH=30/1, v/v) to afford (Racemic)-N-(6-((1,4-dioxan-2-yl)methyl)thiazolo[4,5-b]pyrazin-2-yl)-4-(2-methoxyphenyl)-6-methylnicotinamide (6 mg, 8%) as a yellow solid. LCMS (ESI, m/z): 478 [M+H], ¹H NMR (400 MHz, DMSO-d₆): δ 13.2 (s, 1H), 8.75 (s, 1H), 8.50 (s, 1H), 7.44-7.36 (m, 2H), 7.32 (s, 1H), 7.09 (t, J=7.4 Hz, 1H), 6.97 (d, J=8.6 Hz, 1H), 3.97-3.88 (m, 1H), 3.78 (dd, J=11.4, 2.4 Hz, 1H), 3.73-3.58 (m, 3H), 3.56-3.42 (m, 2H), 3.51 (s, 3H), 2.93 (d, J=6.6 Hz, 2H), 2.58 (s, 3H).

TABLE AP
The following examples were prepared using a similar procedure to that described for Example 212

| Ex. | | | |
|---|---|---|---|
| No. | Structure | Compound Name | Characterization Data |
| 213 | 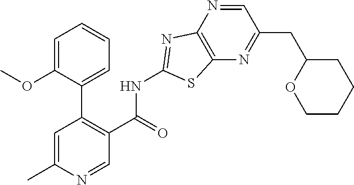 [SMILES: COc1ccccc1-c1cc(C)ncc1C(=O)Nc1nc2ncc(CC3CCCCO3)nc2s1] | (Racemic)-4-(2-Meth- oxyphenyl)-6-methyl- N-(6-((tetrahydro-2H- pyran-2-yl)methyl)- thiazolo[4,5-b]pyrazin- 2-yl)nicotinamide | LCMS (m/z): 476 [M + H]⁺. ¹H NMR (400 MHz, DMSO- d₆) δ 8.74 (s, 1H), 8.48 (s, 1H), 7.42-7.36 (m, 2H), 7.33 (s, 1H), 7.09 (t, J = 7.4 Hz, 1H), 6.97 (d, J = 8.6 Hz, 1H), 3.79 (d, J = 11.0 Hz, 1H), 3.74-3.65 (m, 1H), 3.51 (s, 3H), 3.17 (d, J = 5.2 Hz, 1H), 2.99-2.92 (m, 2H), 2.59 (s, 3H), 1.81-1.73 (m, 1H), 1.67- 1.59 (m, 1H), 1.51-1.40 (m, 4H) |

### Example 214: Synthesis of 4-(5-Cyano-2-methoxyphenyl)-N-(6-cyclopropylimidazo[2,1-b][1,3,4]thiadiazol-2-yl)-6-methylnicotinamide

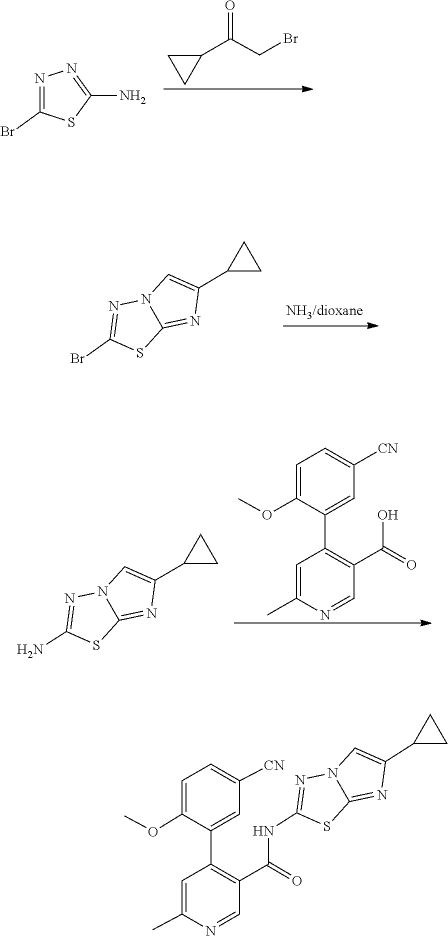 [SMILES: Nc1nnc(Br)s1 ; O=C(CBr)C1CC1 ; Brc1nn2cc(C3CC3)nc2s1 ; Nc1nn2cc(C3CC3)nc2s1 ; COc1ccc(C#N)cc1-c1cc(C)ncc1C(=O)O ; COc1ccc(C#N)cc1-c1cc(C)ncc1C(=O)Nc1nn2cc(C3CC3)nc2s1]

Step 1: To a solution of 5-bromo-1,3,4-thiadiazol-2-amine (3 g, 17 mmol) in water (30 mL) was added 2-bromo-1-cyclopropylethan-1-one (4.1 g, 25 mmol) and the mixture was heated in a sealed tube at 100° C. for 16 h. After cooling to room temperature, the mixture was diluted with water (5 mL), extracted with EtOAc (3×50 mL) and the combined organic layers dried over Na₂SO₄, filtered and concentrated under reduced pressure. The residue was purified by silica gel chromatography (Pet. Ether/EtOAc=10/1, v/v) to give 2-bromo-6-cyclopropylimidazo[2,1-b][1,3,4]thiadiazole (1.8 g, 41%) as a white solid. LCMS (ESI, m/z): 244, 246 [M+H]⁺, ¹H NMR (400 MHz, DMSO-d₆): δ 7.98 (s, 1H), 1.96-1.87 (m, 1H), 0.88-0.82 (m, 2H), 0.76-0.69 (m, 2H).

Step 2: To a solution of 2-bromo-6-cyclopropylimidazo[2,1-b][1,3,4]thiadiazole (1.6 g, 6.6 mmol) in dioxane (32 mL) was added concentrated aqueous NH₄OH (64 mL) and the mixture was heated in a sealed tank at 100° C. for 16 h. After cooling to room temperature, the mixture was concentrated under reduced pressure. The residue was purified by silica gel chromatography (DCM/MeOH=50/1, v/v) and prep-TLC (DCM/MeOH=10/1, v/v) to give 6-cyclopropylimidazo[2,1-b][1,3,4]thiadiazol-2-amine (697 mg, 59%) as a white solid. ¹H NMR (400 MHz, DMSO-d₆): δ 7.40 (s, 1H), 7.29 (s, 2H), 1.84-1.74 (m, 1H), 0.78-0.72 (m, 2H), 0.65-0.61 (m, 2H)

Step 3: To a solution of 6-cyclopropylimidazo[2,1-b][1,3,4]thiadiazol-2-amine (100 mg, 0.55 mmol) in ACN (10 mL) was added 4-(5-cyano-2-methoxyphenyl)-6-methylnicotinic acid (179 mg, 0.66 mmol), TCFH (234 mg, 0.83 mmol) and NMI (182 mg, 2.22 mmol) and the mixture was heated at 80° C. for 22 h. After cooling to room temperature, the mixture was diluted with water (3 mL), extracted with EtOAc (3×20 mL), dried over Na₂SO₄, filtered and concentrated under reduced pressure. The residue was purified by prep-TLC (DCM/MeOH=15/1, v/v) to give 4-(5-cyano-2-methoxyphenyl)-N-(6-cyclopropylimidazo[2,1-b][1,3,4]thiadiazol-2-yl)-6-methylnicotinamide (22 mg, 8%) as a white solid. LCMS (ESI, m/z): 431 [M+H]⁺, ¹H NMR (400 MHz, DMSO-d₆): δ 13.0 (s, 1H), 8.79-8.74 (m, 1H), 7.92-7.88 (m, 1H), 7.87-7.85 (m, 1H), 7.72 (s, 1H), 7.41 (s, 1H), 7.18 (d, J=8.6 Hz, 1H), 3.62 (s, 3H), 2.58 (s, 3H), 1.94-1.84 (m, 1H), 0.85-0.79 (m, 2H), 0.73-0.68 (m, 2H).

### Example 215: Synthesis of 2′-Chloro-N-(6-cyclopropylimidazo[2,1-b][1,3,4]thiadiazol-2-yl)-5′-methoxy-6-methyl-[4,4′-bipyridine]-3-carboxamide

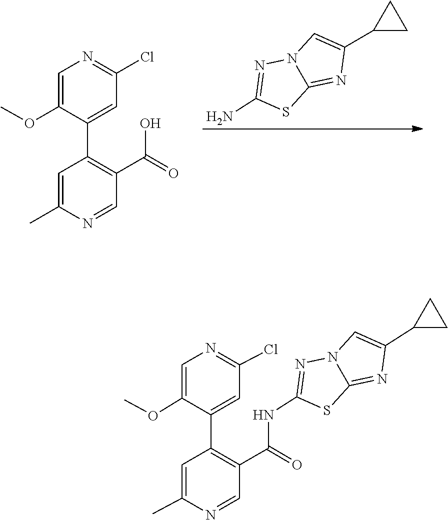 [SMILES: COc1cnc(Cl)cc1-c1cc(C)ncc1C(=O)O ; Nc1nn2cc(C3CC3)nc2s1 ; COc1cnc(Cl)cc1-c1cc(C)ncc1C(=O)Nc1nn2cc(C3CC3)nc2s1]

A solution of 2′-chloro-5′-methoxy-6-methyl-[4,4′-bipyridine]-3-carboxylic acid (95 mg, 0.34 mmol), 6-cyclopropylimidazo[2,1-b][1,3,4]thiadiazol-2-amine (92 mg, 0.51 mmol), HOBt (69.1 mg, 0.51 mmol), EDCI·HCl (98 mg, 0.51 mmol) and DIPEA (220 mg, 1.7 mmol) in DMF (2 mL) was stirred at room temperature overnight. The mixture was diluted with water (20 mL), extracted with EtOAc (4×20 mL) and the combined organic layers washed with brine (20 mL), dried over Na₂SO₄, filtered and concentrated under reduced pressure. The residue was purified by prep-TLC (DCM/MeOH=20/1, v/v) and prep-HPLC to give 2′-chloro-N-(6-cyclopropylimidazo[2,1-b][1,3,4]thiadiazol-2-yl)-5′-methoxy-6-methyl-[4,4′-bipyridine]-3-carboxamide (20 mg, 13%) as a white solid. LCMS (ESI, m/z): 441 [M+H]⁺, ¹H NMR (400 MHz, DMSO-d₆): δ 13.1 (s, 1H), 8.85 (s, 1H), 8.17 (s, 1H), 7.70 (s, 1H), 7.53 (s, 1H), 7.41 (s, 1H), 3.65 (s, 3H), 2.59 (s, 3H), 1.93-1.83 (m, 1H), 0.84-0.77 (m, 2H), 0.73-0.67 (m, 2H).

### Example 216: Synthesis of N3′-(6-Cyclopropylimidazo[2,1-b][1,3,4]thiadiazol-2-yl)-5-methoxy-6′-methyl-[4,4′-bipyridine]-2,3′-dicarboxamide

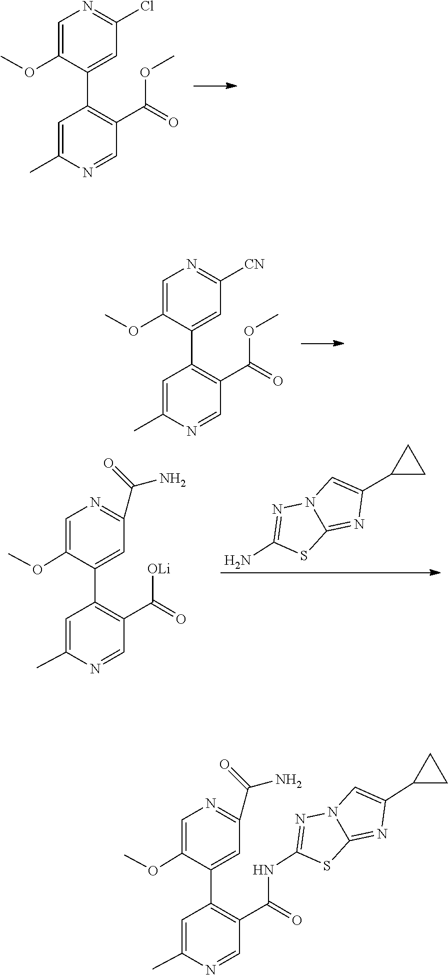

Step 1: To a solution of methyl 2′-chloro-5′-methoxy-6-methyl-[4,4′-bipyridine]-3-carboxylate (300 mg, 1.02 mmol) in DMA (30 mL) was added Zn(CN)₂ (180 mg, 1.54 mmol), [PdCl(allyl)]2 (56 mg, 0.15 mmol) and Xphos (244 mg, 0.51 mmol) and the mixture was heated at 120° C. overnight. The mixture was diluted with water (150 mL), extracted with EtOAc (4×30 mL) and the combined organic layers washed with brine (60 mL), dried over Na₂SO₄, filtered and concentrated under reduced pressure. The residue was purified by prep-TLC (DCM/MeOH=30/1, v/v) to give methyl 2′-cyano-5′-methoxy-6-methyl-[4,4′-bipyridine]-3-carboxylate (243 mg, 83%) as a white solid. LCMS (ESI, m/z): 284 [M+H]+

Step 2: To a solution of methyl 2′-cyano-5′-methoxy-6-methyl-[4,4′-bipyridine]-3-carboxylate (70 mg, 0.18 mmol) in THF (1 mL) and water (1 mL) was added LiOH (12.7 mg, 0.529 mmol) and the mixture was stirred at room temperature for 3 h. The mixture was concentrated under reduced pressure to give lithium 2′-carbamoyl-5′-methoxy-6-methyl-[4,4′-bipyridine]-3-carboxylate (70 mg) as a white solid, which was use directly in the next step. LCMS (ESI, m/z): 288 [M−Li+2H]H.

Step 3: To a solution of lithium 2′-carbamoyl-5′-methoxy-6-methyl-[4,4′-bipyridine]-3-carboxylate (70 mg) in DMF (3 mL) was added 6-cyclopropylimidazo[2,1-b][1,3,4]thiadiazol-2-amine (47 mg, 0.26 mmol), HOBt (52.7 mg, 0.390 mmol), EDCI·HCl (74.8 mg, 0.390 mmol) and DIPEA (168 mg, 1.3 mmol) and the mixture was stirred at room temperature overnight. The mixture was diluted with water (30 mL), extracted with EtOAc (5×30 mL) and the combined organic layers washed with brine (60 mL), dried over Na₂SO₄, filtered and concentrated under reduced pressure. The residue was purified by prep-TLC and prep-HPLC to give N3′-(6-Cyclopropylimidazo[2,1-b][1,3,4]thiadiazol-2-yl)-5-methoxy-6′-methyl-[4,4′-bipyridine]-2,3′-dicarboxamide (2 mg, 3% over two steps) as a white solid. LCMS (ESI, m/z): 450 [M+H], H NMR (400 MHz, DMSO-d₆): δ 13.1 (s, 1H), 8.84 (s, 1H), 8.36 (s, 1H), 8.02 (s, 1H), 7.97 (s, 1H), 7.68 (s, 1H), 7.57 (s, 1H), 7.42 (s, 1H), 3.74 (s, 3H), 2.60 (s, 3H), 1.93-1.83 (m, 1H), 0.85-0.76 (m, 2H), 0.74-0.66 (m, 2H).

### Example 217: Synthesis of 4-(2-(4-(2-Methoxyphenyl)-6-methylnicotinamido)thiazolo[4,5-b]pyrazin-6-yl)pyridine 1-oxide

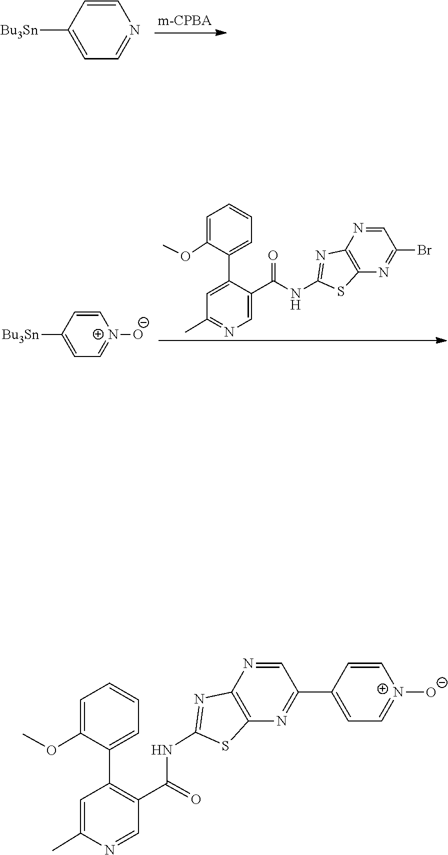

Step 1: To a solution of 4-(tributylstannyl)pyridine (1.2 g, 3.3 mmol) in CHCl₃ (24 mL) at 0° C. was added m-CPBA (1.12 g, 6.52 mmol) and the mixture was stirred at 0° C. for 2 h. The reaction was quenched with a 0.5 M aqueous Na₂SO₂₀₃ solution, extracted with DCM (3×30 mL) and the combined organic phases dried over Na₂SO₄, filtered and concentrated under reduced pressure. The residue was purified by silica gel chromatography (Pet. ether/EtOAc=10/1-1/2, v/v) to give 4-(tributylstannyl)pyridine 1-oxide (958 mg, 76%) as a yellow oil. ¹H NMR (400 MHz, Chloroform-d) δ 8.18 (d, J=6.2 Hz, 2H), 7.40-7.33 (m, 2H), 1.60-1.41 (m, 6H), 1.35-1.26 (m, 6H), 1.19-1.03 (m, 6H), 0.92-0.84 (m, 9H).

Step 2: To a solution of N-(6-bromothiazolo[4,5-b]pyrazin-2-yl)-4-(2-methoxyphenyl)-6-methylnicotinamide (213 mg, 0.46 mmol) in dry toluene (6 mL) was added 4-(tributylstannyl)pyridine 1-oxide (538 mg, 1.40 mmol) and Pd(PPh₃)₄ (54 mg, 0.04 mmol) and the mixture was heated at 110° C. for 16 h under N2. After cooling to room temperature, the mixture was diluted with water (3 mL), extracted with EtOAc (5×15 mL) and the combined organic phases dried over Na₂SO₄, filtered and concentrated under reduced pressure. The residue was purified by prep-HPLC to give 4-(2-(4-(2-methoxyphenyl)-6-methylnicotinamido)thiazolo[4,5-b]pyrazin-6-yl)pyridine 1-oxide (11.7 mg, 5%) as a yellow solid. LCMS (ESI, m/z): 471.20 [M+H], ¹H NMR (400 MHz, DMSO-d₆): δ 13.4 (s, 1H), 9.29 (s, 1H), 8.78 (s, 1H), 8.36-8.30 (m, 2H), 8.23-8.17 (m, 2H), 7.43-7.37 (m, 2H), 7.33 (s, 1H), 7.10 (t, J=7.4 Hz, 1H), 6.98 (d, J=8.4 Hz, 1H), 3.52 (s, 3H), 2.59 (s, 3H).

### Example 218: Synthesis of N-(6-(Azetidine-1-carbonyl)thiazolo[4,5-b]pyrazin-2-yl)-4-(5-cyano-2-methoxyphenyl)-6-methylnicotinamide

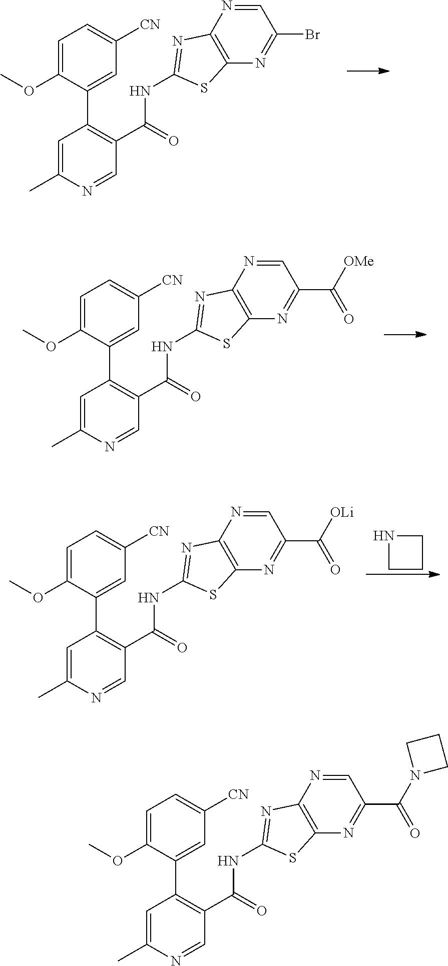

Step 1: To a solution of N-(6-bromothiazolo[4,5-b]pyrazin-2-yl)-4-(5-cyano-2-methoxyphenyl)-6-methylnicotinamide (255 mg, 0.530 mmol) in MeOH (10 mL) was added TEA (107 mg, 1.06 mmol) and Pd(dppf)Cl₂ (38.4 mg, 0.053 mmol) and the mixture was heated at 80° C. overnight under a CO atmosphere (2 MPa). The mixture was diluted with water (30 mL), extracted with EtOAc (40 mL×3) and the combined organic layers washed with brine, dried over Na₂SO₄, filtered and concentrated under reduced pressure. The residue was purified by prep-TLC (DCM/MeOH=30/1, v/v) to give methyl 2-(4-(5-cyano-2-methoxyphenyl)-6-methylnicotinamido)thiazolo[4,5-b]pyrazine-6-carboxylate (136 mg, 55%) as a white solid. LCMS (ESI, m/z): 461 [M+H]⁺, ¹HNMR (400 MHz, DMSO-d₆): δ 8.93 (s, 1H), 8.59 (s, 1H), 7.68-7.63 (m, 2H), 7.18 (s, 1H), 6.92 (d, J=9.2 Hz, 1H), 3.68 (s, 3H), 3.34 (s, 3H), 2.35 (s, 3H).

Step 2: To a solution of methyl 2-(4-(5-cyano-2-methoxyphenyl)-6-methylnicotinamido)thiazolo[4,5-b]pyrazine-6-carboxylate (136 mg, 0.295 mmol) in THF (0.5 mL) and water (0.5 mL) was added LiOH (21.2 mg, 0.886 mmol) and the mixture was stirred at room temperature for 3 h. The mixture was concentrated under reduced pressure to give lithium 2-(4-(5-cyano-2-methoxyphenyl)-6-methylnicotinamido)thiazolo[4,5-b]pyrazine-6-carboxylate (130 mg) as a white solid, which was used directly in the next step. LCMS (ESI, m/z): 447 [M−Li+2H]⁺.

Step 3: To a solution of lithium 2-(4-(5-cyano-2-methoxyphenyl)-6-methylnicotinamido)thiazolo[4,5-b]pyrazine-6-carboxylate (115 mg, 0.254 mmol), azetidine (22.1 mg, 0.386 mmol) and DIPEA (50.7 mg, 0.386 mmol) in DMF (5 mL) at 0° C. was added T₃P (50% in DMF, 491 mg, 0.773 mmol) and the mixture was allowed to warm to room temperature and stirred overnight. The mixture was diluted with water (50 mL), extracted with EtOAc (40 mL×6) and the combined organic layers washed with brine (120 mL), dried over Na₂SO₄, filtered and concentrated under reduced pressure. The residue was purified by prep-TLC (DCM/MeOH=30/1, v/v) to give N-(6-(azetidine-1-carbonyl)thiazolo[4,5-b]pyrazin-2-yl)-4-(5-cyano-2-methoxyphenyl)-6-methylnicotinamide (71.5 mg, 57%) as a white solid. LCMS (ESI, m/z): 486. [M+H]⁺, ¹H NMR (400 MHz, DMSO-d₆): δ 13.6 (s, 1H), 9.08 (s, 1H), 8.82 (s, 1H), 7.94-7.89 (m, 2H), 7.46 (s, 1H), 7.18 (d, J=9.2 Hz, 1H), 4.62 (t, J=7.6 Hz, 2H), 4.12 (t, J=7.6 Hz, 2H), 3.59 (s, 3H), 2.61 (s, 3H), 2.35-2.26 (in, 2H).

TABLE Y
The following examples were prepared using a similar procedure to that described for Example 218

| Ex. | | | |
|---|---|---|---|
| No. | Structure | Compound Name | Characterization Data |
| 219 | 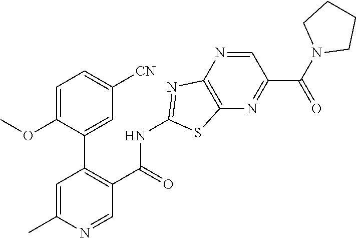 [SMILES: COc1ccc(C#N)cc1-c1cc(C)ncc1C(=O)Nc1nc2ncc(C(=O)N3CCCC3)nc2s1] | 4-(5-Cyano-2-methoxy- phenyl)-6-methyl-N-(6- (pyrrolidine-1-carbon- yl)thiazolo[4,5-b]pyr- azin-2-yl)nicotinamide | LCMS (m/z): 500 [M + H]⁺. ¹H NMR (400 MHz, DMSO- d₆) δ 13.5 (s, 1H), 8.93 (s, 1H), 8.83 (s, 1H), 7.97-7.84 (m, 2H), 7.45 (s, 1H), 7.18 (d, J = 9.2 Hz, 1H), 3.73-3.66 (m, 2H), 3.60 (s, 3H), 3.57- 3.51 (m, 2H), 2.60 (s, 3H), 1.91-1.82 (m, 4H) |
| 220 | 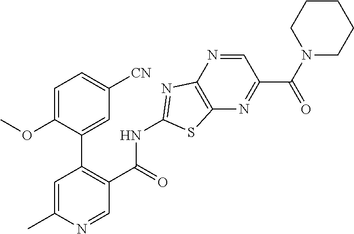 [SMILES: COc1ccc(C#N)cc1-c1cc(C)ncc1C(=O)Nc1nc2ncc(C(=O)N3CCCCC3)nc2s1] | 4-(5-cyano-2-methoxy- phenyl)-6-methyl-N- (6-(piperidine-1-car- bonyl)thiazolo[4,5-b] pyrazin-2-yl)nicotin- amide | LCMS (m/z): 514 [M + H]⁺. ¹H NMR (400 MHz, DMSO- d₆) δ 13.5 (s, 1H), 8.84 (s, 1H), 8.75 (s, 1H), 7.93-7.88 (m, 2H), 7.43 (s, 1H), 7.17 (d, J = 9.2 Hz, 1H), 3.67-3.61 (m, 2H), 3.60 (s, 3H), 3.45- 3.39 (m, 2H), 2.60 (s, 3H), 1.69-1.55 (m, 4H), 1.54-1.44 (m, 2H) |
| 221 | 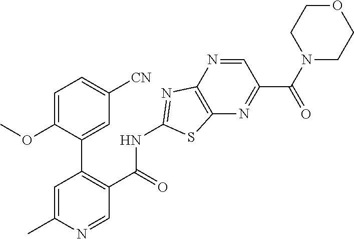 [SMILES: COc1ccc(C#N)cc1-c1cc(C)ncc1C(=O)Nc1nc2ncc(C(=O)N3CCOCC3)nc2s1] | 4-(5-cyano-2-methoxy- phenyl)-6-methyl-N- (6-(morpholine-4-car- bonyl)thiazolo[4,5-b] pyrazin-2-yl)nicotin- amide | LCMS (m/z): 516 [M + H]⁺. ¹H NMR (400 MHz, DMSO- d₆) δ 13.4 (s, 1H), 8.84-8.82 (m, 1H), 7.91-7.86 (m, 2H), 7.44 (s, 1H), 7.18 (d, J = 8.8 Hz, 1H), 3.75-8.52 (m, 8H), 3.62 (s, 3H), 2.62 (s, 3H) |
| 222 | 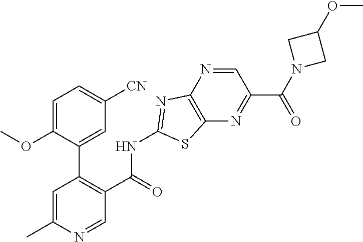 | 4-(5-cyano-2-methoxy- phenyl)-N-(6-(3-meth- oxyazetidine-1-carbon- yl)thiazolo[4,5-b]pyr- azin-2-yl)-6-methyl- nicotinamide | LCMS (m/z): 516 [M + H]⁺. ¹H NMR (400 MHz, DMSO- d₆) δ 13.6 (s, 1H), 9.09 (s, 1H), 8.83 (s, 1H), 7.95-7.88 (m, 2H), 7.46 (s, 1H), 7.24- 7.14 (m, 1H), 4.79 (dd, J = 10.4, 5.6 Hz, 1H), 4.46-4.37 (m, 1H), 4.36-4.22 (m, 2H), 3.97-3.83 (m, 1H), 3.60 (s, 3H), 3.25 (s, 3H), 2.61 (s, 3H) |
| 223 | 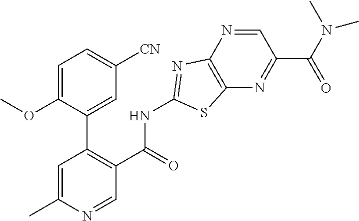 | 2-(4-(5-cyano-2-meth- oxyphenyl)-6-methyl- nicotinamido)-N,N- dimethylthiazolo[4,5- b]pyrazine-6-carbox- amide | LCMS (m/z): 474 [M + H]⁺. ¹H NMR (400 MHz, DMSO- d₆) δ 13.5 (s, 1H), 8.83 (s, 1H), 8.80 (s, 1H), 7.94-7.90 (m, 2H), 7.45 (s, 1H), 7.18 (d, J = 9.2 Hz, 1H), 3.60 (s, 3H), 3.05 (s, 3H), 3.04 (s, 3H), 2.61 (s, 3H) |
| 224 | 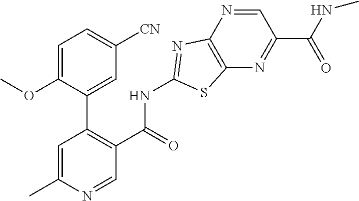 | 2-(4-(5-cyano-2-meth- oxyphenyl)-6-methyl- nicotinamido)-N-meth- ylthiazolo[4,5-b]pyr- azine-6-carboxamide | LCMS (m/z): 460 [M + H]⁺. ¹H NMR (400 MHz, DMSO- d₆) δ 13.6 (s, 1H), 9.15 (s, 1H), 8.95 (d, J = 5.0 Hz, 1H), 8.83 (s, 1H), 7.95-7.90 (m, 2H), 7.46 (s, 1H), 7.19 (d, J = 9.2 Hz, 1H), 3.60 (s, 3H), 2.84 (d, J = 4.6 Hz, 3H), 2.61 (s, 3H) |
| 225 | 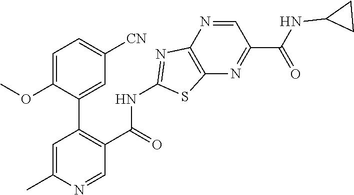 [SMILES: COc1ccc(C#N)cc1-c1cc(C)ncc1C(=O)Nc1nc2ncc(C(=O)NC3CC3)nc2s1] | 2-(4-(5-cyano-2-meth- oxyphenyl)-6-methyl- nicotinamido)-N-cyclo- propylthiazolo[4,5-b] pyrazine-6-carbox- amide | LCMS (m/z): 486 [M + H]⁺. ¹H NMR (400 MHz, DMSO- d₆) δ 13.6 (s, 1H), 9.13 (s, 1H), 8.96 (s, 1H), 8.83 (s, 1H), 7.91 (d, J = 6.4 Hz, 2H), 7.44 (s, 1H), 7.17 (d, J = 9.2 Hz, 1H), 3.59 (s, 3H), 2.98- 2.90 (m, 1H), 2.60 (s, 3H), 0.72-0.68 (m, 4H) |
| 226 | 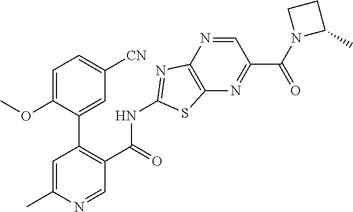 | (S)-4-(5-cyano-2- methoxyphenyl)-6- methyl-N-(6-(2-meth- ylazetidine-1-carbon- yl)thiazolo[4,5-b]pyr- azin-2-yl)nicotinamide | LCMS (m/z): 500 [M + H]⁺. ¹H NMR (400 MHz, DMSO- d₆) δ 13.5 (s, 1H), 9.05-9.01 (m, 1H), 8.85 (s, 1H), 7.92- 7.88 (m, 2H), 7.42 (s, 1H), 7.17 (d, J = 8.5 Hz, 1H), 4.70-4.39 (m, 2H), 4.14-3.93 (m, 1H), 3.60 (s, 3H), 2.60 (s, 3H), 2.06-1.76 (m, 2H), 1.47 (d, J = 6.2 Hz, 2H), 1.39 (d, J = 6.2 Hz, 1H) |
| 227 | 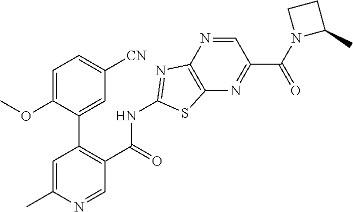 | (R)-4-(5-cyano-2- methoxyphenyl)-6- methyl-N-(6-(2-meth- ylazetidine-1-carbon- yl)thiazolo[4,5-b]pyr- azin-2-yl)nicotinamide | LCMS (m/z): 500 [M + H]⁺. ¹H NMR (400 MHz, DMSO- d₆) δ 13.5 (s, 1H), 9.07-9.04 (m, 1H), 8.83 (s, 1H), 7.94- 7.89 (m, 2H), 7.45 (s, 1H), 7.18 (d, J = 9.2 Hz, 1H), 4.67-4.40 (m, 2H), 4.13-3.94 (m, 1H), 3.60 (s, 3H), 2.61 (s, 3H), 2.03-1.80 (m, 2H), 1.48 (d, J = 6.2 Hz, 2H), 1.39 (d, J = 6.2 Hz, 1H) |
| 228 | 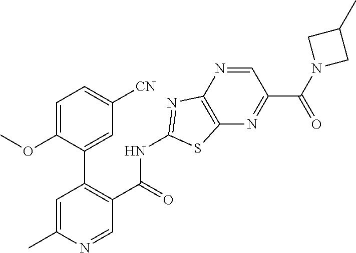 | 4-(5-cyano-2-methoxy- phenyl)-6-methyl-N- (6-(3-methylazetidine- 1-carbonyl)thiazolo [4,5-b]pyrazin-2-yl)- nicotinamide | LCMS (m/z): 500 [M + H]⁺. ¹H NMR (400 MHz, DMSO- d₆) δ 13.6 (s, 1H), 9.08 (s, 1H), 8.83 (s, 1H), 7.92 (d, J = 6.2 Hz, 2H), 7.45 (s, 1H), 7.18 (d, J = 9.2 Hz, 1H), 4.72 (t, J = 9.2 Hz, 1H), 4.26-4.15 (m, 2H), 3.69-3.64 (m, 1H), 3.59 (s, 3H), 2.80-2.72 (m, 1H), 2.61 (s, 3H), 1.24 (s, 3H) |
| 229 | 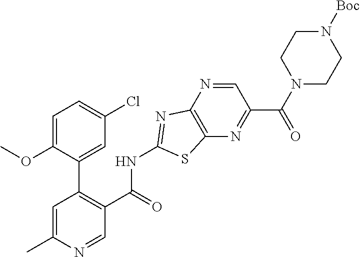 | tert-butyl 4-(2-(4-(5- cyano-2-methoxyphen- yl)-6-methylnicotin- amido)thiazolo[4,5-b] pyrazine-6-carbonyl)- piperazine-1-carbox- ylate | LCMS (m/z): 615 [M + H]⁺. ¹H NMR (400 MHz, DMSO- d₆) δ 13.5 (s, 1H), 8.83 (s, 1H), 8.82 (s, 1H), 7.95-7.88 (m, 2H), 7.45 (s, 1H), 7.22- 7.14 (m, 1H), 3.66 (br s, 2H), 3.60 (s, 3H), 3.53 (br s, 2H), 3.45 (br s, 2H), 3.36 (br s, 2H), 2.61 (s, 3H), 1.41 (s, 9H) |
| 230 | 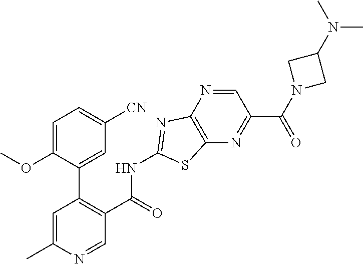 | 4-(5-cyano-2-meth- oxyphenyl)-N-(6-(3- (dimethylamino)- azetidine-1-carbonyl)- thiazolo[4,5-b]pyrazin- 2-yl)-6-methylnicotin- amide | LCMS (m/z): 529 [M + H]⁺. ¹H NMR (400 MHz, DMSO- d₆) δ 13.6 (s, 1H), 9.13 (s, 1H), 8.83 (s, 1H), 7.93 (s, 1H), 7.91 (s, 1H), 7.46 (s, 1H), 7.18 (d, J = 8.6 Hz, 1H), 4.94-4.71 (m, 2H), 4.41-4.24 (m, 2H), 4.20-4.07 (m, 1H), 3.60 (s, 3H), 2.81 (s, 6H), 2.61 (s, 3H) |
| 231 | 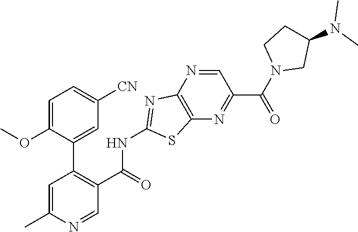 | (R)-4-(5-cyano-2- methoxyphenyl)-N-(6- (3-(dimethylamino)- pyrrolidine-1-carbon- yl)thiazolo[4,5-b]pyr- azin-2-yl)-6-methyl- nicotinamide | LCMS (m/z): 543 [M + H]⁺. ¹H NMR (400 MHz, DMSO- d₆) δ 8.98 (s, 1H), 8.82 (s, 1H), 7.90-7.88 (m, 1H), 7.87 (s, 1H), 7.44 (s, 1H), 7.17 (d, J = 8.6 Hz, 1H), 4.08-3.90 (m, 2H), 3.88-3.73 (m, 2H), 3.66-3.55 (m, 1H), 3.62 (s, 3H), 2.87 (s, 3H), 2.81 (s, 3H), 2.61 (s, 3H), 2.42-2.33 (m, 1H), 2.21-2.09 (m, 1H) |
| 232 | 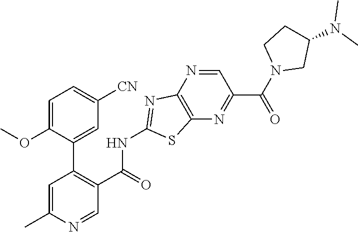 | (S)-4-(5-cyano-2- methoxyphenyl)-N-(6- (3-(dimethylamino)- pyrrolidine-1-carbon- yl)thiazolo[4,5-b]pyr- azin-2-yl)-6-methyl- nicotinamide | LCMS (m/z): 543 [M + H]⁺. ¹H NMR (400 MHz, DMSO- d₆) δ 8.84 (s, 1H), 8.00 (s, 1H), 8.14 (s, 1H), 7.84-7.78 (m, 1H), 7.75 (s, 1H), 7.32 (s, 1H), 7.12 (d, J = 8.6 Hz, 1H), 4.17-3.88 (m, 2H), 3.67-3.55 (m, 3H), 3.58 (s, 3H), 2.67 (s, 3H), 2.61 (s, 3H), 2.57 (s, 3H), 2.32-2.24 (m, 1H), 2.09- 1.97 (m, 1H) |

### Example 233: Synthesis of 4-(5-Cyano-2-methoxyphenyl)-6-methyl-N-(6-(2-oxopiperidin-1-yl)thiazolo[4,5-b]pyrazin-2-yl)nicotinamide

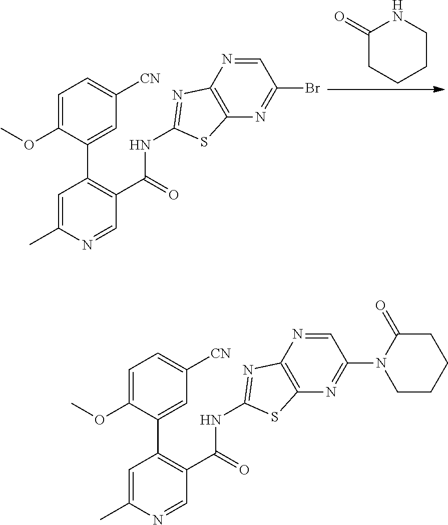 [SMILES: COc1ccc(C#N)cc1-c1cc(C)ncc1C(=O)Nc1nc2ncc(Br)nc2s1 ; O=C1CCCCN1 ; COc1ccc(C#N)cc1-c1cc(C)ncc1C(=O)Nc1nc2ncc(N3CCCCC3=O)nc2s1]

To a solution of N-(6-bromothiazolo[4,5-b]pyrazin-2-yl)-4-(5-cyano-2-methoxyphenyl)-6-methylnicotinamide (100 mg, 0.10 mmol) and piperidin-2-one (24 mg, 0.22 mmol) in 1,4-dioxane (4 mL) was added Pd₂(dba)₃ (9 mg, 0.01 mmol), Xantphos (12 mg, 0.02 mmol) and Cs₂CO₃(101 mg, 0.31 mmol) and the mixture was heated at 100° C. in a sealed tube for 24 h under a N₂ atmosphere. The mixture was diluted with water (50 mL), extracted with EtOAc (30 mL×4) and the combined organic layers washed with brine (30 mL), dried over Na₂SO₄, filtered and concentrated under reduced pressure. The residue was purified by prep-TLC (DCM/MeOH=20/1, v/v) to afford 4-(5-cyano-2-methoxyphenyl)-6-methyl-N-(6-(2-oxopiperdin-1-yl)thiazolo[4,5-b]pyrazin-2-yl)nicotinamide (30 mg, 300) as a light yellow solid. LCMS (ESI, m/z): 500 [M+H]⁺, ¹HNMR (400 MHz, DMSO-d₆): δ 13.3 (s, 1H), 8.88 (s, 1H), 8.81 (s, 1H), 7.93-7.88 (m, 2H), 7.44 (s, 1H), 7.17 (d, J=9.2 Hz, 1H), 3.86 (t, J 5.8 Hz, 2H), 3.59 (s, 3H), 2.60 (s, 3H), 2.55-2.50 (m, 2H), 1.93-1.82 (i, 4H).

TABLE Z
The following examples were prepared using a similar procedure to that described for Example 233

| Ex. | | | |
|---|---|---|---|
| No. | Structure | Compound Name | Characterization Data |
| 234 | 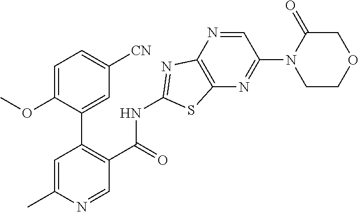 [SMILES: COc1ccc(C#N)cc1-c1cc(C)ncc1C(=O)Nc1nc2ncc(N3CCOCC3=O)nc2s1] | 4-(5-Cyano-2-meth- oxyphenyl)-6-methyl- N-(6-(3-oxomorpho- lino)thiazolo[4,5-b] pyrazin-2-yl)nicotin- amide | LCMS (m/z): 502 [M + H]⁺. ¹H NMR (400 MHz, DMSO- d₆) δ 13.3 (s, 1H), 9.18 (s, 1H), 8.81 (s, 1H), 7.91 (dd, J = 4.6, 2.6 Hz, 2H), 7.44 (s, 1H), 7.17 (d, J = 9.2 Hz, 1H), 4.33 (s, 2H), 4.06-3.96 (m, 4H), 3.59 (s, 3H), 2.60 (s, 3H) |
| 235 | 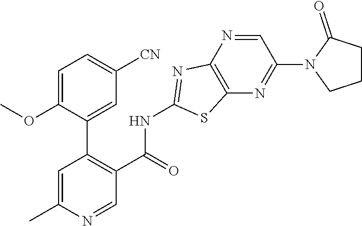 [SMILES: COc1ccc(C#N)cc1-c1cc(C)ncc1C(=O)Nc1nc2ncc(N3CCCC3=O)nc2s1] | 4-(5-Cyano-2-meth- oxyphenyl)-6-meth- yl-N-(6-(2-oxopyr- rolidin-1-yl)thiazolo [4,5-b]pyrazin-2-yl)- nicotinamide | LCMS (m/z): 486 [M + H]⁺. ¹H NMR (400 MHz, DMSO- d₆) δ 13.3 (s, 1H), 9.52 (s, 1H), 8.80 (s, 1H), 7.95-7.88 (m, 2H), 7.44 (s, 1H), 7.17 (d, J = 9.2 Hz, 1H), 4.02 (t, J = 7.0 Hz, 2H), 3.59 (s, 3H), 2.63 (t, J = 8.0 Hz, 2H), 2.60 (s, 3H), 2.16-2.07 (m, 2H) |
| 236 | 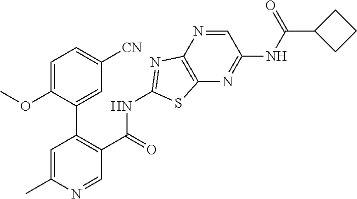 [SMILES: COc1ccc(C#N)cc1-c1cc(C)ncc1C(=O)Nc1nc2ncc(NC(=O)C3CCC3)nc2s1] | 4-(5-Cyano-2-meth- oxyphenyl)-N-(6- (cyclobutanecarbox- amido)thiazolo[4,5- b]pyrazin-2-yl)-6- methylnicotinamide | LCMS (m/z): 500 [M + H]+. ¹H NMR (400 MHz, DMSO- d₆) δ 13.3 (s, 1H), 10.8 (s, 1H), 9.35 (s, 1H), 8.80 (s, 1H), 7.96-7.85 (m, 2H), 7.43 (s, 1H), 7.18 (d, J = 8.6 Hz, 1H), 3.59 (s, 3H), 3.43-3.36 (m, 1H), 2.60 (s, 3H), 2.28- 2.18 (m, 2H), 2.17-2.05 (m, 2H), 2.01-1.94 (m, 2H) |
| 237 | 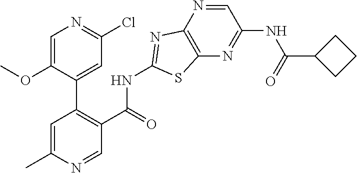 [SMILES: COc1cnc(Cl)cc1-c1cc(C)ncc1C(=O)Nc1nc2ncc(NC(=O)C3CCC3)nc2s1] | 2′-Chloro-N-(6- cyclobutanecarbox- amido)thiazolo[4,5- b]pyrazin-2-yl)-5′- methoxy-6-methyl- [4,4′-bipyridine]-3- carboxamide | LCMS (m/z): 510 [M + H]⁺. ¹H NMR (400 MHz, DMSO- d₆) δ 13.4 (s, 1H), 10.8 (s, 1H), 9.37 (s, 1H), 8.87 (s, 1H), 8.17 (s, 1H), 7.59 (s, 1H), 7.47 (s, 1H), 3.63 (s, 3H), 3.44-3.36 (m, 1H), 2.61 (s, 3H), 2.30-2.19 (m, 2H), 2.19-2.09 (m, 2H), 2.01-1.90 (m, 1H), 1.88-1.78 (m, 1H) |

### Example 238: Synthesis of 4-(5-Cyano-2-methoxyphenyl)-6-methyl-N-(6-(piperazine-1-carbonyl)thiazolo[4,5-b]pyrazin-2-yl)nicotinamide hydrochloride

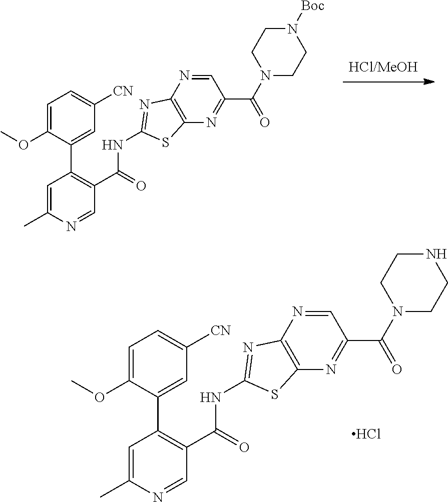 [SMILES: COc1ccc(C#N)cc1-c1cc(C)ncc1C(=O)Nc1nc2ncc(C(=O)N3CCN(C(=O)OC(C)(C)C)CC3)nc2s1 ; COc1ccc(C#N)cc1-c1cc(C)ncc1C(=O)Nc1nc2ncc(C(=O)N3CCNCC3)nc2s1 ; Cl]

A mixture of tert-butyl 4-(2-(4-(5-cyano-2-methoxyphenyl)-6-methylnicotinamido)thiazolo[4,5-b]pyrazine-6-carbonyl)piperazine-1-carboxylate (49 mg, 0.08 mmol) and 4 M HCl/MeOH (5 mL) was stirred at room temperature for 2 h. The mixture was concentrated under reduced pressure to give 4-(5-cyano-2-methoxyphenyl)-6-methyl-N-(6-(piperazine-1-carbonyl)thiazolo[4,5-b]pyrazin-2-yl)nicotinamide hydrochloride (30 mg, 68%) as an off-white solid. LCMS (ESI, m/z): 515 [M+H]⁺, ¹H NMR (400 MHz, DMSO-d₆): δ 13.6 (s, 1H), 9.56 (br s, 2H), 8.90 (s, 1H), 8.89 (s, 1H), 7.97-7.92 (m, 2H), 7.60 (s, 1H), 7.20 (d, J=8.4 Hz, 1H), 3.96-3.82 (m, 4H), 3.60 (s, 3H), 3.26-3.07 (m, 4H), 2.66 (s, 3H).

### Example 239: Synthesis of 4-(7-Methoxy-1-oxo-1,2,3,4-tetrahydroisoquinolin-6-yl)-6-methyl-N-(6-(pyridin-4-yl)thiazolo[4,5-b]pyrazin-2-yl)nicotinamide

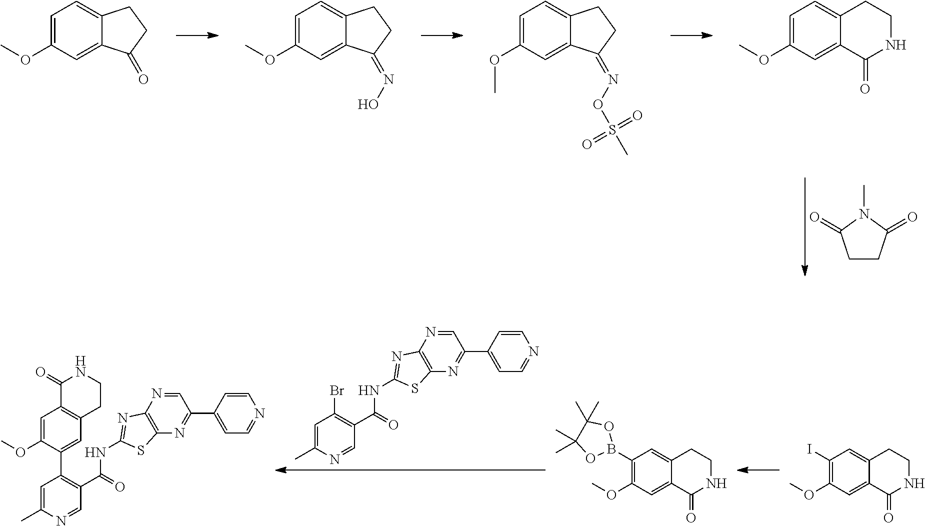

Step 1: To a solution of 6-methoxy-2,3-dihydro-1H-inden-1-one (500 mg, 3.08 mmol) in MeOH (7 mL) at room temperature was added TEA (0.86 mL, 6.16 mmol) and NH₂OH·HCl (300 mg, 4.42 mmol) and the mixture was stirred at room temperature overnight. The mixture was diluted with water (20 mL), extracted with EtOAc (2×20 mL) and the combined organic phases washed with water (20 mL), brine (20 mL), dried over Na₂SO₄, filtered and concentrated under reduced pressure. The residue was purified by trituration (Pet. ether/EtOAc=10/1, v/v) to give 6-methoxy-2,3-dihydro-1H-inden-1-one oxime (512 mg, 94%) as a white solid. LCMS (ESI, m/z): 178 [M+H]⁺. ¹H NMR (400 MHz, DMSO-d₆) δ 7.48 (d, J=8.4 Hz, 1H), 7.25 (dd, J=8.4, 2.6 Hz, 1H), 7.08 (d, J=2.6 Hz, 1H), 3.79 (d, J=0.8 Hz, 3H), 3.03-2.98 (m, 2H), 2.66-2.62 (m, 2H).

Step 2: To a solution of 6-methoxy-2,3-dihydro-1H-inden-1-one oxime (200 mg, 1.13 mmol) in DCM (4 mL) at 0° C. was added TEA (0.63 mL, 4.52 mmol), DMAP (14 mg, 0.11 mmol) and MsCl (0.13 mL, 1.7 mmol) and the mixture was stirred at room temperature overnight. The mixture was diluted with water (10 mL), extracted with EtOAc (2×20 mL) and the combined organic phases were washed with a saturated aqueous NH₄Cl solution (10 mL), brine (10 mL), dried over Na₂SO₄, filtered and concentrated under reduced pressure. The residue was purified by prep-TLC (Pet. Ether/DCM=1/1, v/v) to give 6-methoxy-2,3-dihydro-1H-inden-1-one O-methylsulfonyl oxime (236 mg, 82%) as a black solid. LCMS (ESI, m/z): 256 [M+H]⁺.

Step 3: A solution of 6-methoxy-2,3-dihydro-TH-inden-1-one O-methylsulfonyl oxime (1.00 g, 3.92 mmol), BF₃ (55-60% w/w in MeOH, 748 mg, 6.27 mmol), TiCl₄ (1.19 g, 6.27 mmol) and MsCl (772 mg, 6.74 mmol) in DCE (20 mL) was stirred at room temperature overnight under a N₂ atmosphere. The mixture was diluted with DCM (100 mL) and a saturated aqueous NaHCO₃ solution to pH 7, and the combined organic layers were washed with brine (50 mL), dried over Na₂SO₄, filtered, and concentrated under reduced pressure. The residue was purified by silica gel chromatography (10-50% EtOAc in Pet. ether) to afford 7-methoxy-3,4-dihydroisoquinolin-1(2H)-one (500 mg, 72%) as a gray solid. LCMS (ESI, m/z): 178 [M+H]⁺. ¹H NMR (400 MHz, Chloroform-d) δ 7.59 (d, J=2.8 Hz, 1H), 7.13 (d, J=8.2 Hz, 1H), 7.01 (dd, J=8.4, 2.8 Hz, 1H), 6.16 (s, 1H), 3.85 (s, 3H), 3.55 (td, J=6.6, 2.8 Hz, 2H), 2.93 (t, J=6.6 Hz, 2H).

Step 4: To a solution of 7-methoxy-3,4-dihydroisoquinolin-1(2H)-one (300 mg, 1.7 mmol) in conc. H₂SO₄ (3 mL) at 0° C. was added NIS (438 mg, 1.9 mmol) portion-wise and the mixture was allowed to warm to room temperature and stirred for 14 h. The mixture was cooled to 0° C., adjusted to pH 8 with aqueous NaOH and extracted with EtOAc (5×30 mL).

The combined organic layers were washed with brine (3×30 mL), dried over Na₂SO₄, filtered and concentrated under reduced pressure. The residue was purified by prep-TLC (Pet. Ether/EtOAc=1/1) to give 6-iodo-7-methoxy-3,4-dihydroisoquinolin-1(2H)-one (243 mg, 47%) as a yellow solid. LCMS (ESI, m/z): 304 [M+H]⁺. ¹H NMR (400 MHz, DMSO-d₆) δ 7.99 (s, 1H), 7.77 (s, 1H), 7.34 (s, 1H), 3.85 (s, 3H), 3.41-3.36 (m, 2H), 2.81 (t, J=6.6 Hz, 2H).

Step 5: A solution of 6-iodo-7-methoxy-3,4-dihydroisoquinolin-1(2H)-one (200 mg, 0.66 mmol), Pin₂B₂ (502.8 mg, 1.98 mmol), KOAc (194.3 mg, 1.98 mmol) and Pd(dppf)Cl₂ (47.9 mg, 0.066 mmol) in dioxane (10 mL) was heated at 100° C. overnight under a N₂ atmosphere.

The mixture was diluted with EtOAc (20 mL), washed with water (2×30 mL), brine (10 mL), dried over Na₂SO₄, filtered and concentrated under reduced pressure. The residue was purified by prep-TLC (DCM/MeOH=20/1, v/v) to give 7-methoxy-6-(4,4,5,5-tetramethyl-1,3,2-dioxaborolan-2-yl)-3,4-dihydroisoquinolin-1(2H)-one (167 mg, 84%) as a yellow solid. LCMS (ESI, m/z):304 [M+H]⁺. ¹H NMR (400 MHz, DMSO-d₆) δ 7.99 (s, 1H), 7.44 (s, 1H), 7.35 (s, 1H), 3.76 (s, 3H), 3.42 (s, 2H), 2.82 (t, J=6.6 Hz, 2H), 1.28 (s, 12H).

Step 6: To a solution of ethyl 4-bromo-6-methyl-N-(6-(pyridin-4-yl)thiazolo[4,5-b]pyrazin-2-yl)nicotinamide (25 mg, 0.059 mmol), 7-methoxy-6-(4,4,5,5-tetramethyl-1,3,2-dioxaborolan-2-yl)-3,4-dihydroisoquinolin-1(2H)-one (22 mg, 0.070 mmol) and Na₂CO₃ (19 mg, 0.18 mmol) in dioxane (1.5 mL) and water (0.3 mL) under a N₂ atmosphere was added Pd(dppf)Cl₂ (4.3 mg, 0.006 mmol) and the mixture was heated at 130° C. in a sealed tube under microwave irradiation for 2 h. The mixture was concentrated under reduced pressure and the residue diluted with water (30 mL) and extracted with EtOAc (2×30 mL). The combined organic layers were washed with water (30 mL), brine (30 mL), dried over Na₂SO₄, filtered and concentrated under reduced pressure. The residue was purified by prep-TLC (DCM/MeOH=15/1, v/v) to afford 4-(7-methoxy-1-oxo-1,2,3,4-tetrahydroisoquinolin-6-yl)-6-methyl-N-(6-(pyridin-4-yl)thiazolo[4,5-b]pyrazin-2-yl)nicotinamide (3 mg, 10%) as a green solid. LCMS (ESI, m/z): 524 [M+H]⁺, ¹H NMR (400 MHz, DMSO-d₆) δ 13.5 (s, 1H), 9.32 (s, 1H), 8.84 (s, 1H), 8.73 (d, J=4.8 Hz, 2H) 8.13 (d, J=4.8 Hz, 2H), 8.02 (s, 1H), 7.42-7.33 (m, 3H), 3.54 (s, 3H), 3.45-3.39 (m, 2H), 2.96-2.90 (m, 2H), 2.60 (s, 3H).

### Example 240: Synthesis of (R)-2-(2′-Chloro-5′-methoxy-6-methyl-[4,4′-bipyridine]-3-carboxamido)-N-(tetrahydrofuran-3-yl)imidazo[2,1-b][1,3,4]thiadiazole-6-carboxamide

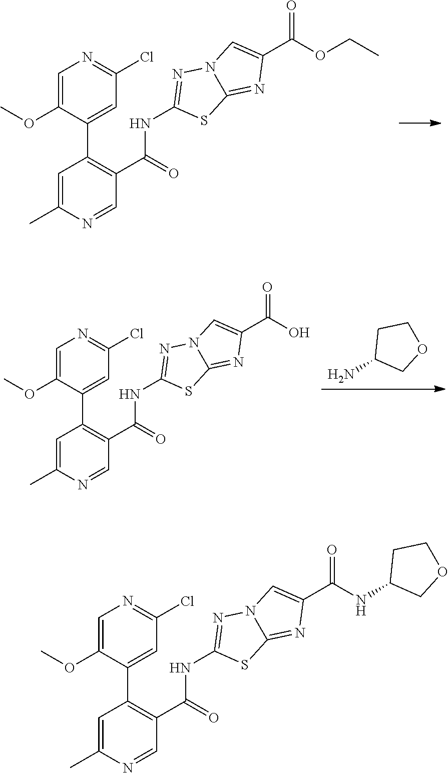

Step 1: To a solution of ethyl 2-(2′-chloro-5′-methoxy-6-methyl-[4,4′-bipyridine]-3-carboxamido)imidazo[2,1-b][1,3,4]thiadiazole-6-carboxylate (50 mg, 0.11 mmol) in THF (1 mL) and water (1 mL) was added NaOH (13.1 mg, 0.327 mmol) and the mixture was heated at 50° C. for 5 h. The mixture was adjusted to pH 7-8 with a 6 M aqueous HCl solution and concentrated under reduced pressure to give 2-(2′-chloro-5′-methoxy-6-methyl-[4,4′-bipyridine]-3-carboxamido)imidazo[2,1-b][1,3,4]thiadiazole-6-carboxylic acid (48 mg), which was used directly in the step directly. LCMS m/z=445 [M+H]⁺.

Step 2: To a solution of 2-(2′-chloro-5′-methoxy-6-methyl-[4,4′-bipyridine]-3-carboxamido)imidazo[2,1-b][1,3,4]thiadiazole-6-carboxylic acid (48 mg, 0.11 mmol), (R)-tetrahydrofuran-3-amine (18.8 mg, 0.216 mmol) and DIPEA (69.7 mg, 0.539 mmol) in DMF (2 mL) was added at 0° C. was added T3P (50% in DMF, 68.7 mg, 0.108 mmol) and the mixture was stirred at RT overnight. The mixture was diluted with water (20 mL), extracted with EtOAc (20 ml×3) and the combined organic layers washed with brine, dried over Na₂SO₄, filtered and concentrated under reduced pressure. The residue was purified by prep-TLC (DCM/MeOH=15/1, v/v) to afford (R)-2-(2′-chloro-5′-methoxy-6-methyl-[4,4′-bipyridine]-3-carboxamido)-N-(tetrahydrofuran-3-yl)imidazo[2,1-b][1,3,4]thiadiazole-6-carboxamide (29 mg, 52%) as a yellow solid. LCMS (ESI, m/z): 514 [M+H]⁺; ¹H NMR (400 MHz, DMSO-d₆) δ 13.4 (s, 1H), 8.85 (s, 1H), 8.46 (s, 1H), 8.23-8.15 (m, 2H), 7.58 (s, 1H), 7.47 (s, 1H), 4.49-4.38 (m, 1H), 3.87-3.79 (m, 2H), 3.74-3.68 (m, 1H), 3.67 (s, 3H), 3.55 (dd, J=8.8, 4.8 Hz, 1H), 2.60 (s, 3H), 2.17-2.06 (m, 1H), 1.99-1.89 (m, 1H).

TABLE AA
The following examples were prepared using a similar procedure to that described for Example 240

| Ex. | | | |
|---|---|---|---|
| No. | Structure | Compound Name | Characterization Data |
| 241 | 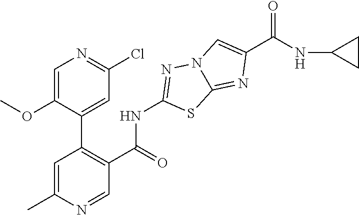 [SMILES: COc1cnc(Cl)cc1-c1cc(C)ncc1C(=O)Nc1nn2cc(C(=O)NC3CC3)nc2s1] | 2-(2′-chloro-5′-methoxy- 6-methyl-[4,4′-bipyr- idine]-3-carboxamido)- N-cyclopropylimidazo [2,1-b][1,3,4]thiadia- zole-6-carboxamide | LCMS (m/z): 484 [M + H]⁺. ¹H NMR (400 MHz, DMSO- d₆) δ 13.4 (s, 1H), 8.86 (s, 1H), 8.39 (s, 1H), 8.19 (s, 1H), 8.15 (d, J = 4.8 Hz, 1H), 7.56 (s, 1H), 7.45 (s, 1H), 3.67 (s, 3H), 2.87-2.78 (m, 1H), 2.60 (s, 3H), 0.68-0.56 (m, 4H) |
| 242 | 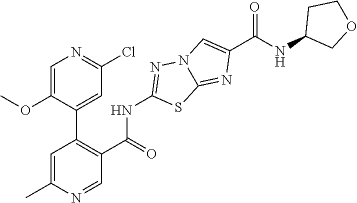 [SMILES: COc1cnc(Cl)cc1-c1cc(C)ncc1C(=O)Nc1nn2cc(C(=O)N[C@H]3CCOC3)nc2s1] | (S)-2-(2′-chloro-5′- methoxy-6-methyl-[4,4′- bipyridine]-3-carbox- amido)-N-(tetrahydro- furan-3-yl)imidazo[2,1- b][1,3,4]thiadiazole-6- carboxamide | LCMS (m/z): 514 [M + H]⁺. ¹H NMR (400 MHz, DMSO- d₆) δ 13.4 (s, 1H), 8.86 (s, 1H), 8.44 (s, 1H), 8.21-8.16 (m, 2H), 7.57 (s, 1H), 7.46 (s, 1H), 4.51-4.36 (m, 1H), 3.89-3.79 (m, 2H), 3.75-3.69 (m, 1H), 3.67 (s, 3H), 3.58- 3.51 (m, 1H), 2.60 (s, 3H), 2.19-2.06 (m, 1H), 2.01-1.89 (m, 1H) |
| 243 | 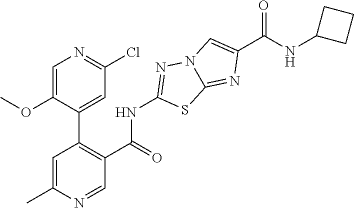 [SMILES: COc1cnc(Cl)cc1-c1cc(C)ncc1C(=O)Nc1nn2cc(C(=O)NC3CCC3)nc2s1] | 2-(2′-chloro-5′-meth- oxy-6-methyl-[4,4′- bipyridine]-3-carbox- amido)-N-cyclobutyl- imidazo[2,1-b][1,3,4] thiadiazole-6-carbox- amide | LCMS (m/z): 498 [M + H]⁺. ¹H NMR (400 MHz, DMSO- d₆) δ 13.4 (s, 1H), 8.85 (s, 1H), 8.41 (s, 1H), 8.34 (d, J = 8.6 Hz, 1H), 8.20 (s, 1H), 7.58 (s, 1H), 7.47 (s, 1H), 4.46-4.35 (m, 1H), 3.67 (s, 3H), 2.60 (s, 3H), 2.18-2.07 (m, 4H), 1.66-1.58 (m, 2H) |
| 244 | 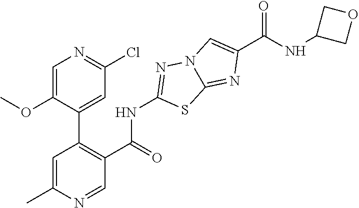 [SMILES: COc1cnc(Cl)cc1-c1cc(C)ncc1C(=O)Nc1nn2cc(C(=O)NC3COC3)nc2s1] | 2-(2′-chloro-5′-meth- oxy-6-methyl-[4,4′- bipyridine]-3-carbox- amido)-N-(oxetan-3-yl)- imidazo[2,1-b][1,3,4] thiadiazole-6-carbox- amide | LCMS (m/z): 500 [M + H]⁺. ¹H NMR (400 MHz, DMSO- d₆) δ 13.4 (s, 1H), 8.94 (d, J = 7.0 Hz, 1H), 8.85 (s, 1H), 8.47 (s, 1H), 8.20 (s, 1H), 7.58 (s, 1H), 7.48 (s, 1H), 5.06-4.95 (m, 1H), 4.70 (t, J = 6.4 Hz, 2H), 4.63 (t, J = 6.4 Hz, 2H), 3.67 (s, 3H), 2.61 (s, 3H) |

### Example 245: Synthesis of (Racemic)-2′-Chloro-5′-methoxy-6-methyl-N-(6-(tetrahydrofuran-3-yl)thiazolo[4,5-b]pyridin-2-yl)-[4,4′-bipyridine]-3-carboxamide

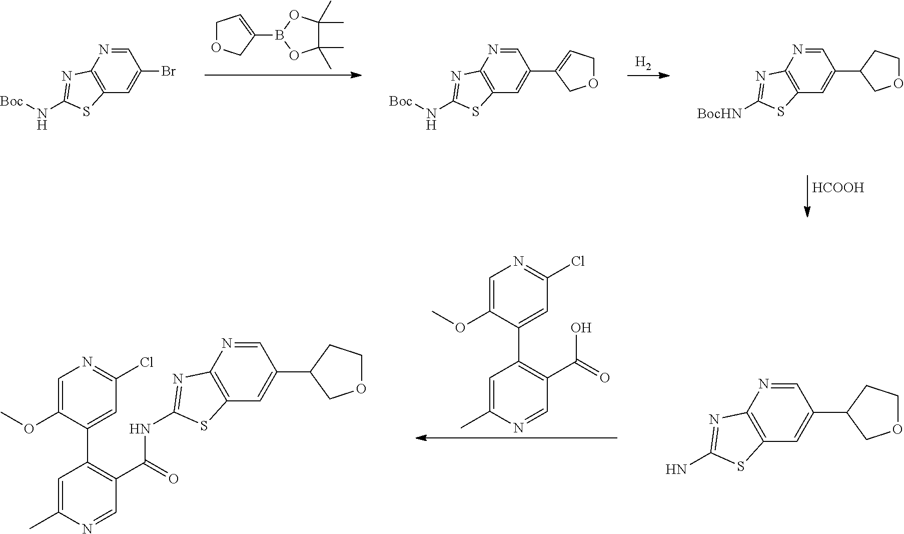

Step 1: To a solution of tert-butyl (6-bromothiazolo[4,5-b]pyridin-2-yl)carbamate (200 mg, 0.606 mmol), 2-(2,5-dihydrofuran-3-yl)-4,4,5,5-tetramethyl-1,3,2-dioxaborolane (128 mg, 0.909 mmol) and Na₂CO₃ (193 mg, 1.82 mmol) in dioxane (2 mL) and water (0.4 mL) under a N₂ atmosphere was added Pd(dppf)Cl₂ (44 mg, 0.061 mmol) and the mixture was heated at 100° C. overnight. The mixture was concentrated under reduced pressure and the residue diluted with water (50 mL) and extracted with EtOAc (2×50 mL). The combined organic layers were washed with water (50 mL), brine (50 mL), dried over Na₂SO₄, filtered and concentrated under reduced pressure. The residue was purified by prep-TLC (DCM/MeOH=20/1, v/v) to afford tert-butyl (6-(2,5-dihydrofuran-3-yl)thiazolo[4,5-b]pyridin-2-yl)carbamate (135 mg, 70%) as a yellow solid. LCMS (ESI, m/z): 320 [M+H]⁺, ¹H NMR (400 MHz, DMSO-d₆) δ 12.1 (s, 1H), 8.61 (d, J=2.2 Hz, 1H), 8.44 (d, J=2.2 Hz, 1H), 6.75-6.57 (m, 1H), 4.96 (td, J=5.0, 2.4 Hz, 2H), 4.76 (td, J=4.8, 1.8 Hz, 2H), 1.52 (s, 9H).

Step 2: A mixture of tert-butyl (6-(2,5-dihydrofuran-3-yl)thiazolo[4,5-b]pyridin-2-yl)carbamate (135 mg, 0.423 mmol) and 10% Pd/C (70 mg) in MeOH (20 mL) was stirred at room temperature under a H₂ atmosphere (23 atm) for 2 days. The mixture was filtered and the filtrate concentrated under reduced pressure to afford tert-butyl (6-(tetrahydrofuran-3-yl)thiazolo[4,5-b]pyridin-2-yl)carbamate (90 mg, 66%) as a yellow solid. LCMS (ESI, m/z): 322 [M+H]⁺, ¹H NMR (400 MHz, DMSO-d₆) δ 12.0 (s, 1H), 8.39 (d, J=2.2 Hz, 1H), 8.28 (d, J=2.2 Hz, 1H), 4.06 (t, J=7.6 Hz, 1H), 3.99 (td, J=8.2, 4.6 Hz, 1H), 3.82 (q, J=7.8 Hz, 1H), 3.60 (t, J=7.8 Hz, 1H), 3.50 (p, J=7.6 Hz, 1H), 2.43-2.29 (m, 1H), 1.99 (dt, J=12.2, 7.8 Hz, 1H), 1.51 (s, 9H).

Step 3: A solution of tert-butyl (6-(tetrahydrofuran-3-yl)thiazolo[4,5-b]pyridin-2-yl)carbamate (90 mg, 0.28 mmol) in formic acid (3 mL) was stirred at room temperature for 5 h. The mixture was diluted with water and freeze-dried to afford 6-(tetrahydrofuran-3-yl)thiazolo[4,5-b]pyridin-2-amine (50 mg, 80%) as an off-white solid. LCMS (ESI, m/z):221.90 [M+H]⁺.

Step 4: To a mixture of 2′-chloro-5′-methoxy-6-methyl-[4,4′-bipyridine]-3-carboxylic acid (300 mg, 1.08 mmol), TCFH (453 mg, 1.61 mmol) and NMI (354 mg, 4.31 mmol) in ACN (10 mL) was added 6-(tetrahydrofuran-3-yl)thiazolo[4,5-b]pyridin-2-amine (238 mg, 1.08 mmol) and the mixture was heated at 80° C. overnight. The mixture was concentrated under reduced pressure and the residue diluted with water (50 mL) and extracted with EtOAc (2×50 mL). The combined organic layers were washed with water (50 mL), brine (50 mL), dried over Na₂SO₄, filtered and concentrated under reduced pressure. The residue was purified by prep-TLC (DCM/MeOH=20/1, v/v) to give (Racemic)-2′-chloro-5′-methoxy-6-methyl-N-(6-(tetrahydrofuran-3-yl)thiazolo[4,5-b]pyridin-2-yl)-[4,4′-bipyridine]-3-carboxamide (60 mg, 12%) as a white solid. LCMS (ESI, m/z): 482 [M+H]⁺, ¹H NMR (400 MHz, DMSO-d₆) δ 8.86 (s, 1H), 8.49 (d, J=2.2 Hz, 1H), 8.37 (d, J=2.2 Hz, 1H), 8.16 (s, 1H), 7.59 (s, 1H), 7.47 (s, 1H), 4.07 (t, J=7.6 Hz, 1H), 4.00 (td, J=8.2, 4.6 Hz, 1H), 3.83 (q, J=7.8 Hz, 1H), 3.62 (t, J=7.6 Hz, 1H), 3.59 (s, 3H), 3.57-3.50 (m, 1H), 2.61 (s, 3H), 2.43-2.34 (m, 1H), 2.04-1.94 (m, 1H).

### Example 292A & 292B: Synthesis of (R or S)-2′-chloro-5′-methoxy-6-methyl-N-(6-(tetrahydrofuran-3-yl)thiazolo[4,5-b]pyridin-2-yl)-[4,4′-bipyridine]-3-carboxamide (292A) & (S or R)-2′-chloro-5′-methoxy-6-methyl-N-(6-(tetrahydrofuran-3-yl)thiazolo[4,5-b]pyridin-2-yl)-[4,4′-bipyridine]-3-carboxamide (292B)

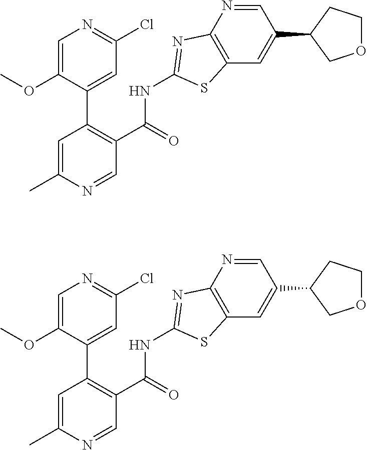 [SMILES: COc1cnc(Cl)cc1-c1cc(C)ncc1C(=O)Nc1nc2ncc([C@H]3CCOC3)cc2s1 ; COc1cnc(Cl)cc1-c1cc(C)ncc1C(=O)Nc1nc2ncc([C@@H]3CCOC3)cc2s1]

Racemic example 245 (40 mg) was separated by prep-chiral-HPLC (CHIRAL ART Cellulose-SZ column, 2×25 cm, 5 μm; 20%/80% 2:1 MeCN:EtOH/Hexane with 0.1% formic acid modifier; 20 mL/min flow rate; wave length: 259/290 nm) to afford two peaks:

Peak 1 RT=18.2 min to afford (R or S)-2′-chloro-5′-methoxy-6-methyl-N-(6-(tetrahydrofuran-3-yl)thiazolo[4,5-b]pyridin-2-yl)-[4,4′-bipyridine]-3-carboxamide (9.3 mg, 23% yield) as a white solid. LCMS (ESI, m/z): 482 [M+H]⁺. ¹H NMR (300 MHz, DMSO-d₆) δ 8.87 (s, 1H), δ 8.49 (s, 1H), δ 8.36 (s, 1H), δ 8.16 (s, 1H), δ 7.59 (s, 1H), δ 7.46 (s, 1H), δ 4.09-4.05 (m, 1H), δ 4.02-3.86 (m, 1H), δ 3.84-3.80 (m, 1H), δ 3.63-3.59 (m, 4H), δ 3.57-3.51 (m, 1H), δ 2.61 (s, 3H), δ 2.49-2.35 (m, 1H), δ 2.09-1.91 (m, 1H)

Peak 2 RT=22.6 min to afford (S or R)-2′-chloro-5′-methoxy-6-methyl-N-(6-(tetrahydrofuran-3-yl)thiazolo[4,5-b]pyridin-2-yl)-[4,4′-bipyridine]-3-carboxamide (13.4 mg, 33% yield) as a white solid. LCMS (ESI, m/z): 482 [M+H]⁺. ¹H NMR (300 MHz, DMSO-d₆) δ 8.87 (s, 1H), δ 8.49 (s, 1H), δ 8.36 (s, 1H), δ 8.16 (s, 1H), δ 7.59 (s, 1H), δ 7.46 (s, 1H), δ 4.09-4.06 (m, 1H), δ 4.02-3.86 (m, 1H), δ 3.84-3.80 (m, 1H), δ 3.63-3.59 (m, 4H), δ 3.57-3.52 (m, 1H), δ 2.61 (s, 3H), δ 2.49-2.37 (m, 1H), δ 2.09-1.89 (m, 1H).

### Example 246: Synthesis of 2′-Chloro-5′-methoxy-6-methyl-N-(6-(2-oxopiperidin-1-yl)thiazolo[4,5-b]pyridin-2-yl)-[4,4′-bipyridine]-3-carboxamide

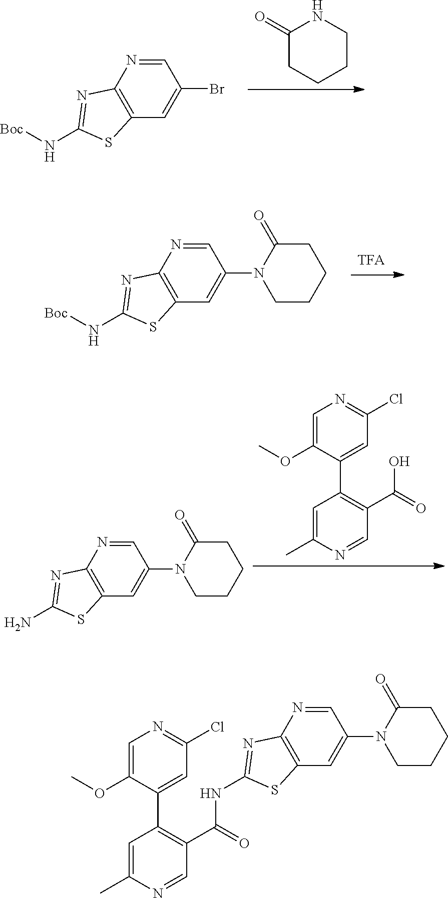

Step 1: To a solution of tert-butyl (6-bromothiazolo[4,5-b]pyridin-2-yl)carbamate (250 mg, 0.755 mmol), piperidin-2-one (225 mg, 2.26 mmol) and Cs₂CO₃ (492 mg, 1.51 mmol) in DMF (3 mL) was added CuI (288 mg, 1.51 mmol) and N,N′-dimethylethylenediamine (266 mg, 3.02 mmol) and the mixture was heated at 110° C. in a sealed tube under N₂ overnight. The mixture was diluted with water (50 mL), extracted with EtOAc (50 mL×2) and the combined organic layers washed with water (50 mL), brine (50 mL), dried over Na₂SO₄, filtered and concentrated under reduced pressure. The residue was purified by prep-TLC (DCM/MeOH=15/1, v/v) to afford tert-butyl (6-(2-oxopiperidin-1-yl)thiazolo[4,5-b]pyridin-2-yl)carbamate (50 mg, 19%) as a yellow solid. LCMS (ESI, m/z): 349 [M+H]⁺, ¹H NMR (400 MHz, DMSO-d₆) δ 12.1 (s, 1H), 8.40 (s, 1H), 8.33 (s, 1H), 3.66 (s, 2H), 2.43 (t, J=6.2 Hz, 2H), 1.95-1.84 (m, 4H), 1.52 (s, 9H).

Step 2: To a solution of tert-butyl (6-(2-oxopiperidin-1-yl)thiazolo[4,5-b]pyridin-2-yl)carbamate (80 mg, 0.23 mmol) in DCM (4 mL) was added TFA (2 mL) and the mixture was stirred at room temperature for 3 h. The mixture was diluted with water (30 mL), adjusted to pH 8 with a saturated aqueous NaHCO₃ solution and extracted with EtOAc (30 mL×2). The combined organic layers were washed with water (30 mL), brine (30 mL), dried over Na₂SO₄, filtered and concentrated under reduced pressure. The residue was purified by silica gel chromatography (DCM/MeOH=10/1, v/v) to afford 1-(2-aminothiazolo[4,5-b]pyridin-6-yl)piperidin-2-one (30 mg, 52%) as an off-white solid. LCMS (ESI, m/z): 249 [M+H]⁺, ¹H NMR (400 MHz, DMSO-d₆) δ 8.11 (s, 1H), 7.98 (s, 1H), 7.94 (s, 2H), 3.65-3.55 (m, 2H), 2.44-2.36 (m, 2H), 1.94-1.79 (m, 4H).

Step 3: To a mixture of 2′-chloro-5′-methoxy-6-methyl-[4,4′-bipyridine]-3-carboxylic acid (50 mg, 0.18 mmol), TCFH (84 mg, 0.30 mmol) and NMI (59 mg, 0.72 mmol) in ACN (5 mL) was added 1-(2-aminothiazolo[4,5-b]pyridin-6-yl)piperidin-2-one (30 mg, 0.12 mmol) and mixture was heated at 80° C. overnight. The mixture was concentrated under reduced pressure and the residue diluted with water (30 mL) and extracted with EtOAc (2×30 mL). The combined organic layers were washed with water (30 mL), brine (30 mL), dried over Na₂SO₄, filtered and concentrated under reduced pressure. The residue was purified by prep-TLC (DCM/MeOH=15/1, v/v) to give 2′-chloro-5′-methoxy-6-methyl-N-(6-(2-oxopiperidin-1-yl)thiazolo[4,5-b]pyridin-2-yl)-[4,4′-bipyridine]-3-carboxamide (9 mg, 14%) as an off-white solid. LCMS (ESI, m/z): 509 [M+H]⁺, ¹H NMR (400 MHz, DMSO-d₆) δ 8.88 (s, 1H), 8.48 (d, J=2.4 Hz, 1H), 8.38 (d, J=2.4 Hz, 1H), 8.15 (s, 1H), 7.59 (s, 1H), 7.47 (s, 1H), 3.68 (t, J=6.8 Hz, 2H), 3.60 (s, 3H), 2.61 (s, 3H), 2.44 (t, J=6.8 Hz, 2H), 1.97-1.83 (m, 4H).

### Example 247: Synthesis of 2′-Chloro-5′-methoxy-6-methyl-N-(6-(pyrrolidine-1-carbonyl)thiazolo[4,5-b]pyrazin-2-yl)-[4,4′-bipyridine]-3-carboxamide

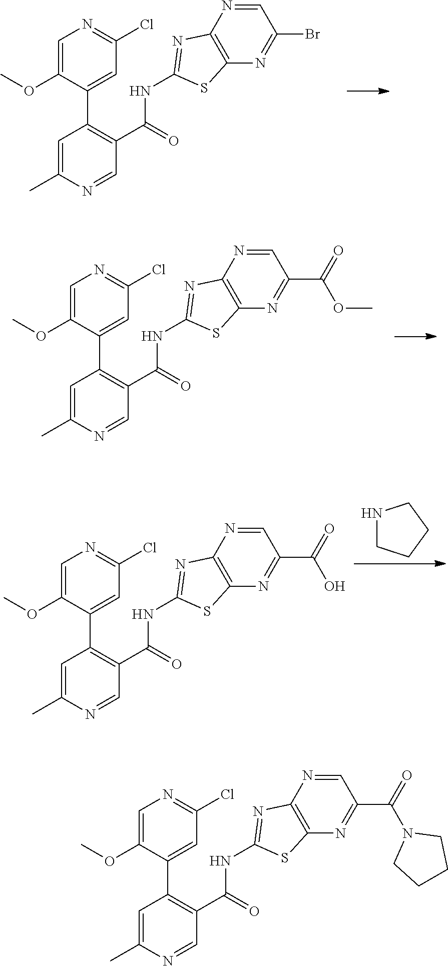

Step 1: A mixture of N-(6-bromothiazolo[4,5-b]pyrazin-2-yl)-2′-chloro-5′-methoxy-6-methyl-[4,4′-bipyridine]-3-carboxamide (200 mg, 0.406 mmol), TEA (82 mg, 0.81 mmol) and Pd(dppf)Cl₂ (30 mg, 0.040 mmol) in MeOH (8 mL) was heated at 80° C. under a CO atmosphere (2 MPa) overnight. The mixture was filtered and the filtrate concentrated under reduced pressure. The residue was purified by prep-TLC (DCM/MeOH=30/1, v/v) to afford methyl 2-(2′-chloro-5′-methoxy-6-methyl-[4,4′-bipyridine]-3-carboxamido)thiazolo[4,5-b]pyrazine-6-carboxylate (100 mg, 52%) as a brown solid. LCMS (ESI, m/z): 471 [M+H]⁺, ¹H NMR (400 MHz, DMSO-d₆) δ 13.8 (s, 1H), 9.21 (s, 1H), 8.90 (s, 1H), 8.18 (s, 1H), 7.62 (s, 1H), 7.49 (s, 1H), 3.95 (s, 3H), 3.63 (s, 3H), 2.62 (s, 3H).

Step 2: A mixture of methyl 2-(2′-chloro-5′-methoxy-6-methyl-[4,4′-bipyridine]-3-carboxamido)thiazolo[4,5-b]pyrazine-6-carboxylate (60 mg, 0.13 mmol) and NaOH (15 mg, 0.38 mmol) in THF (3 mL) and water (3 mL) was heated at 50° C. for 4 h. The solvent was removed under reduced pressure and the residue dissolved in DMF (3 mL) and cooled to 0° C. Pyrrolidine (14 mg, 0.190 mmol), DIPEA (82 mg, 0.634 mmol) and T3P (50% in DMF, 121 mg, 0.190 mmol) were added and the mixture was allowed to warm to room temperature and stirred overnight. The mixture was diluted with water (60 mL), extracted with EtOAc (50 mL×3) and the combined organic layers washed with brine (60 mL), dried over Na₂SO₄, filtered and concentrated under reduced pressure. The residue was purified by prep-TLC (DCM/MeOH=25/1, v/v) to afford 2′-chloro-5′-methoxy-6-methyl-N-(6-(pyrrolidine-1-carbonyl)thiazolo[4,5-b]pyrazin-2-yl)-[4,4′-bipyridine]-3-carboxamide (40 mg, 61%) as a white solid. LCMS (ESI, m/z):510 [M+H], ¹H NMR (400 MHz, DMSO-d₆) δ 13.6 (s, 1H), 8.95 (s, 1H), 8.90 (s, 1H), 8.18 (s, 1H), 7.61 (s, 1H), 7.50 (s, 1H), 3.73-3.67 (m, 2H), 3.63 (s, 3H), 3.58-3.51 (m, 2H), 2.62 (s, 3H), 1.92-1.83 (m, 4H).

TABLE AB
The following example was prepared using a similar procedure to that described for Example 247

| Ex. | | | |
|---|---|---|---|
| No. | Structure | Compound Name | Characterization Data |
| 248 | 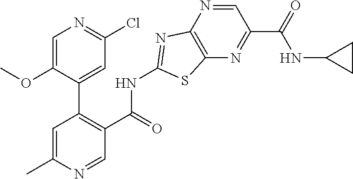 [SMILES: COc1cnc(Cl)cc1-c1cc(C)ncc1C(=O)Nc1nc2ncc(C(=O)NC3CC3)nc2s1] | 2-(2′-chloro-5′-meth- oxy-6-methyl-[4,4′- bipyridine]-3-carbox- amido)-N-cycloprop- ylthiazolo[4,5-b]pyr- azine-6-carboxamide | LCMS (m/z): 496 [M + H]⁺. ¹H NMR (400 MHz, DMSO- d₆) δ 13.7 (s, 1H), 9.16 (s, 1H), 8.99 (d, J = 4.9 Hz, 1H), 8.89 (s, 1H), 8.18 (s, 1H), 7.62 (s, 1H), 7.50 (s, 1H), 3.62 (s, 3H), 2.98-2.90 (m, 1H), 2.62 (s, 3H), 0.73-0.68 (m, 4H) |

### Example 249: Synthesis of 2′-Chloro-N-(6-(cyclobutanecarboxamido)thiazolo[4,5-b]pyridin-2-yl)-5′-methoxy-6-methyl-[4,4′-bipyridine]-3-carboxamide

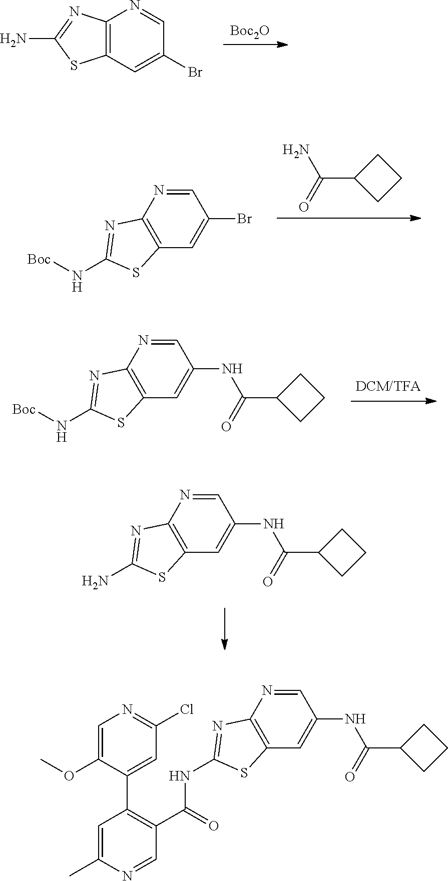

Step 1: To a solution of 6-bromothiazolo[4,5-b]pyridin-2-amine (10 g, 43.5 mmol) in DMF (2 mL) was added di-tert-butyl dicarbonate (19 g, 87 mmol), TEA (13.2 g, 130.4 mmol) and DMAP (1.06 g, 8.69 mmol) and the mixture was stirred at room temperature for 2 h. The mixture was diluted with water (30 mL), extracted with EtOAc (3×30 mL) and the combined organic layers washed with water (30 mL), brine (20 mL), dried over Na₂SO₄, filtered and concentrated under reduced pressure. The residue was purified by silica gel chromatography (100% Pet. Ether to 33% EtOAc/Pet. ether) to give tert-butyl (6-bromothiazolo[4,5-b]pyridin-2-yl)carbamate (9.00 g, 65%) as a yellow solid. LCMS (ESI, m/z): 330, 332 [M+H]⁺, ¹H NMR (400 MHz, DMSO-d₆) δ 12.2 (s, 1H), 8.68 (d, J=2.2 Hz, 1H), 8.56 (d, J=2.4 Hz, 1H), 1.52 (s, 9H).

Step 2: To a solution of tert-butyl (6-bromothiazolo[4,5-b]pyridin-2-yl)carbamate (200 mg, 0.61 mmol) in DMF (2 mL) was added cyclobutanecarboxamide (180 mg, 1.82 mmol), Cs₂CO₃ (395 mg, 1.21 mmol), CuI (231 mg, 1.21 mmol) and N,N′-dimethyl-1,2-ethanediamine (214 mg, 2.42 mmol) and the mixture was heated at 110° C. in a sealed tube under N₂ for 16 h. After cooling to room temperature, the mixture was diluted with water (30 mL), extracted with EtOAc (3×20 mL) and the combined organic phases dried over Na₂SO₄, filtered and concentrated under reduced pressure. The residue was purified by prep-TLC (DCM/MeOH=30/1, v/v) to give tert-butyl (6-(cyclobutanecarboxamido)thiazolo[4,5-b]pyridin-2-yl)carbamate (85 mg, 20%) as a green solid. LCMS (ESI, m/z): 349 [M+H]⁺, ¹H NMR (400 MHz, DMSO-d₆) δ 11.9 (s, 1H), 10.0 (s, 1H), 8.78-8.68 (m, 1H), 8.57-8.46 (m, 1H), 3.29-3.22 (m, 1H), 2.29-2.19 (m, 2H), 2.17-2.09 (m, 2H), 2.01-1.81 (m, 2H), 1.51 (s, 9H).

Step 3: A solution of tert-butyl (6-(cyclobutanecarboxamido)thiazolo[4,5-b]pyridin-2-yl)carbamate (85 mg, 0.24 mmol) and TFA (2 mL) in DCM (4 mL) was stirred at room temperature for 2 h. The mixture was concentrated under reduced pressure to give N-(2-aminothiazolo[4,5-b]pyridin-6-yl)cyclobutanecarboxamide (90 mg, 96%) as a gray solid. LCMS (ESI, m/z): 249 [M+H]⁺.

Step 4: To a solution of 2′-chloro-5′-methoxy-6-methyl-[4,4′-bipyridine]-3-carboxylic acid (50 mg, 0.18 mmol) in ACN (5 mL) was added N-(2-aminothiazolo[4,5-b]pyridin-6-yl)cyclobutanecarboxamide (53 mg, 0.22 mmol), TCFH (75 mg, 0.27 mmol) and NMI (59 mg, 0.72 mmol) and the mixture was heated at 80° C. for 16 h. After cooling to room temperature, the mixture was diluted with water (20 mL), extracted with EtOAc (3×25 mL) and the combined organic phases dried over Na₂SO₄, filtered and concentrated under reduced pressure. The residue was purified by prep-TLC (DCM/MeOH=10/1, v/v) to give 2′-chloro-N-(6-(cyclobutanecarboxamido)thiazolo[4,5-b]pyridin-2-yl)-5′-methoxy-6-methyl-[4,4′-bipyridine]-3-carboxamide (20 mg, 22%) as an off-white solid. LCMS (ESI, m/z): 509 [M+H]⁺, ¹H NMR (400 MHz, DMSO-d₆) δ 13.1 (s, 1H), 10.1 (s, 1H), 8.88 (s, 1H), 8.73 (s, 1H), 8.60 (s, 1H), 8.16 (s, 1H), 7.56 (s, 1H), 7.43 (s, 1H), 3.61 (s, 3H), 3.29-3.22 (m, 1H), 2.60 (s, 3H), 2.31-2.20 (m, 2H), 2.19-2.07 (m, 2H), 2.02-1.92 (m, 1H), 1.90-1.76 (m, 1H).

### Example 250: Synthesis of 4-(5-cyano-2-methoxyphenyl)-6-methyl-N-(5-oxo-7-(pyridin-4-yl)-5H-[1,3,4]thiadiazolo[3,2-a]pyrimidin-2-yl)nicotinamide

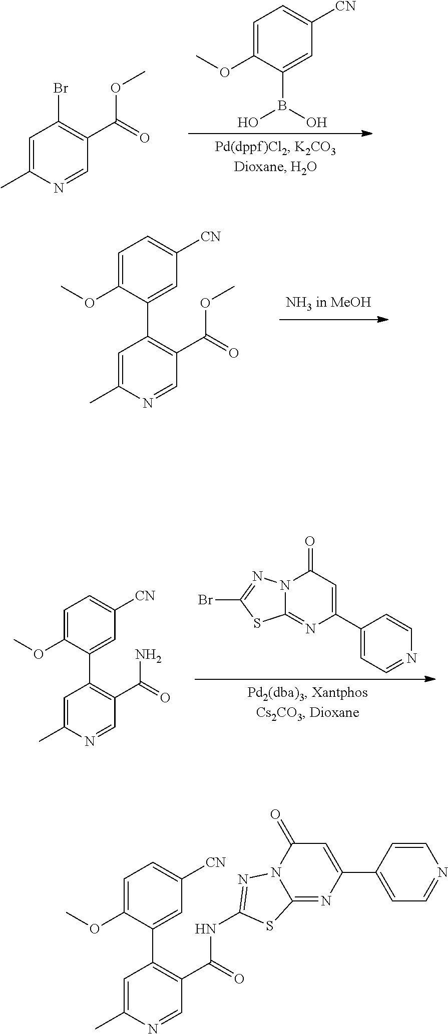

Step 1: A solution of methyl 4-bromo-6-methylpyridine-3-carboxylate (1.0 g, 4.3 mmol), 5-cyano-2-methoxyphenylboronic acid (0.7 g, 4.3 mmol), potassium carbonate (1.8 g, 13 mmol) and Pd(dppf)Cl₂ (0.3 g, 0.4 mmol) in 1,4-dioxane (10 mL) and H₂O (1 mL) was stirred at 90° C. for 16 h under a nitrogen atmosphere. The resulting mixture was cooled to 25° C. and concentrated under reduced pressure. The residue was purified by reverse-phase flash chromatography (10-50% ACN/water with 0.1% formic acid modifier) to afford methyl 4-(5-cyano-2-methoxyphenyl)-6-methylpyridine-3-carboxylate (800 mg, 65.2% yield) as a yellow solid. LCMS (ESI, m/z): [M+H]⁺=283.

Step 2: A solution of methyl 4-(5-cyano-2-methoxyphenyl)-6-methylpyridine-3-carboxylate (500 mg, 1.8 mmol) and NH₃ (15 mL, 7 mol/L in MeOH) was stirred at 80° C. for 16 h. The resulting mixture was cooled to 25° C. and concentrated under reduce pressure. The residue was purified by silica gel column chromatography, eluted with DCM/MeOH (20/1) to afford 4-(5-cyano-2-methoxyphenyl)-6-methylpyridine-3-carboxamide (200 mg, 42.2% yield) as a yellow solid. LCMS (ESI, m/z): [M+H]=268.

Step 3: A solution of 4-(5-cyano-2-methoxyphenyl)-6-methylpyridine-3-carboxamide (200 mg, 0.7 mmol), 2-bromo-7-(pyridin-4-yl)-[1,3,4]thiadiazolo[3,2-a]pyrimidin-5-one (230 mg, 0.7 mmol), cesium carbonate (733 mg, 2.1 mmol), Pd₂(dba)₃ (68 mg, 0.07 mmol) and Xantphos (86 mg, 0.14 mmol) in 1,4-dioxane (4 mL) was stirred at 90° C. for 16 h under nitrogen atmosphere. The resulting mixture was cooled to 25° C. and concentrated under reduced pressure. The residue was purified by reverse-phase flash chromatography (10-50% ACN/water with 0.1% formic acid modifier). The crude product (100 mg) was purified by prep-HPLC (49-66% MeOH in water with 0.1% formic acid modifier) to afford 4-(5-cyano-2-methoxyphenyl)-6-methyl-N-[5-oxo-7-(pyridin-4-yl)-[1,3,4]thiadiazolo[3,2-a]pyrimidin-2-yl]pyridine-3-carboxamide (29.4 mg, 7.9% yield) as a yellow solid. LCMS (ESI, m/z): [M+H]+=496. ¹H NMR (300 MHz, DMSO-d₆) δ13.68 (s, 1H), 8.82 (s, 1H), 8.77-8.61 (m, 2H), 8.10-7.99 (m, 2H), 7.94-7.92 (m, 2H), 7.43 (s, 1H), 7.26-7.08 (m, 2H), 3.67 (s, 3H), 2.60 (s, 3H).

### Example 251: Synthesis of 2′-chloro-5′-methoxy-6-methyl-N-[6-(2-methylpyridin-4-yl)-thiazolo[4,5-b]pyrazin-2-yl]-[4,4′-bipyridine]-3-carboxamide

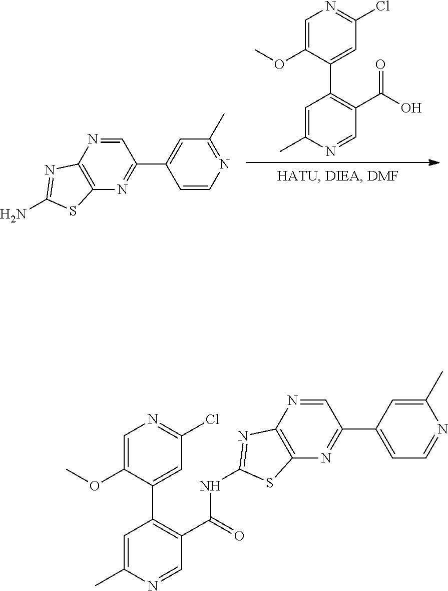

A solution of 2′-chloro-5′-methoxy-6-methyl-[4,4′-bipyridine]-3-carboxylic acid (170 mg, 0.6 mmol) and 6-(2-methylpyridin-4-yl)-[1,3]thiazolo[4,5-b]pyrazin-2-amine (148 mg, 0.6 mmol) in DMF (3 mL) was treated with DIPEA (236 mg, 1.8 mmol) for 5 minutes at 25° C. followed by the addition of HATU (348 mg, 0.9 mmol) in portions at 25° C. The reaction mixture was stirred at 50° C. for 12 h. The resulting mixture was cooled to 25° C. and purified directly by prep-HPLC (9-27% ACN/water with 0.1% formic acid modifier) to afford 2′-chloro-5′-methoxy-6-methyl-N-[6-(2-methylpyridin-4-yl)-[1,3]thiazolo[4,5-b]pyrazin-2-yl]-[4,4′-bipyridine]-3-carboxamide as a formic acid salt (21.0 mg, 6.2% yield) as a yellow solid. LCMS (ESI): [M+H]⁺=504. ¹H NMR (300 MHz, DMSO-d₆) δ 13.59 (s, 1H), 12.75 (s, 1H), 9.34 (s, 1H), 8.92 (s, 1H), 8.60 (d, J=5.3 Hz, 1H), 8.18 (s, 1H), 8.13 (s, 1H), 8.03 (d, J=1.7 Hz, 1H), 7.94 (d, J=1.7 Hz, 1H), 7.61 (s, 1H), 7.48 (s, 1H), 3.64 (s, 3H), 2.62 (s, 3H), 2.58 (s, 3H).

TABLE AC
The following examples were prepared using a similar procedure to that described for Example 251

| Ex. | | | |
|---|---|---|---|
| No. | Structure | Compound Name | Characterization Data |
| 252 | 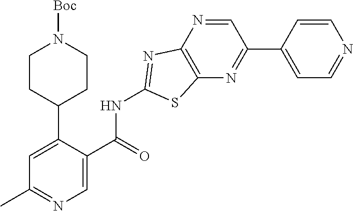 [SMILES: Cc1cc(C2CCN(C(=O)OC(C)(C)C)CC2)c(C(=O)Nc2nc3ncc(-c4ccncc4)nc3s2)cn1] | tert-butyl 4-(2-methyl- 5-((6-(pyridin-4-yl)- thiazolo[4,5-b]pyr- azin-2-yl)carbamoyl)- pyridin-4-yl)piperi- dine-1-carboxylate | LCMS (m/z): 530 [M − H]⁻. ¹H NMR (400 MHz, DMSO- d₆) δ 13.60 (s, 1H), 9.28 (s, 1H), 8.77-8.71 (m, 3H), 8.16- 8.13 (m, 2H), 7.38 (s, 1H), 7.08 (s, 1H), 4.09-4.07 (m, 2H), 3.35 (s, 3H), 2.77 (s, 2H), 1.80-1.77 (m, 2H), 1.61- 1.52 (m, 2H), 1.41 (s, 9H) |
| 253 | 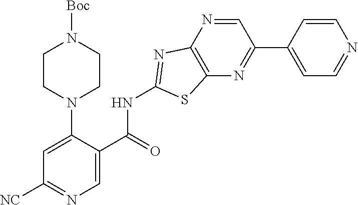 [SMILES: CC(C)(C)OC(=O)N1CCN(c2cc(C#N)ncc2C(=O)Nc2nc3ncc(-c4ccncc4)nc3s2)CC1] | tert-butyl 4-(2-cyano- 5-((6-(pyridin-4-yl)- thiazolo[4,5-b]pyrazin- 2-yl)carbamoyl)pyr- idin-4-yl)piperazine- 1-carboxylate | LCMS (m/z): 544 [M + H]⁺. ¹H NMR (400 MHz, DMSO- d₆) δ 13.67 (s, 1H), 9.44 (s, 1H), 8.81-8.79 (m, 2H), 8.59 (s, 1H), 8.26-8.23 (m, 2H), 7.73 (s, 1H), 3.16 (m, 8H), 1.38 (s, 9H) |
| 254 | 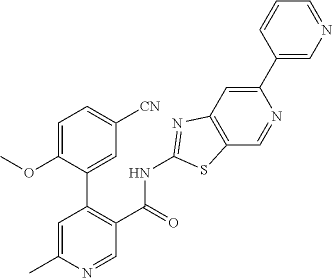 [SMILES: COc1ccc(C#N)cc1-c1cc(C)ncc1C(=O)Nc1nc2cc(-c3cccnc3)ncc2s1] | 4-(5-cyano-2-meth- oxyphenyl)-6-methyl- N-(6-(pyridin-3-yl)- thiazolo[5,4-c]pyridin- 2-yl)nicotinamide | LCMS (m/z): 479 [M + H]⁺. ¹H NMR (400 MHz, DMSO- d₆) δ 13.25 (s, 1H), 9.35 (s, 2H), 8.86 (s, 1H), 8.65-8.59 (m, 2H), 8.55-8.32 (s, 1H), 7.93-7.85 (m, 2H), 7.54-7.51 (m, 2H), 7.17 (d, J = 8.6 Hz, 1H), 3.57 (s, 3H), 2.59 (s, 3H) |
| 255 | 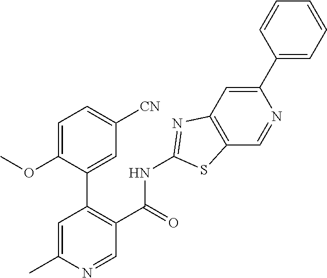 [SMILES: COc1ccc(C#N)cc1-c1cc(C)ncc1C(=O)Nc1nc2cc(-c3ccccc3)ncc2s1] | 4-(5-cyano-2-meth- oxyphenyl)-6-methyl- N-(6-phenylthiazolo [5,4-c]pyridin-2-yl)- nicotinamide | LCMS (m/z): 478 [M + H]⁺. ¹H NMR (400 MHz, DMSO- d₆) δ 13.22 (s, 1H), 9.22 (s, 1H), 8.83 (s, 1H), 8.27-8.13 (m, 3H), 7.92-7.89 (m, 2H), 7.51 (t, J = 7.5 Hz, 4H), 7.17 (d, J = 8.6 Hz, 1H), 3.57 (s, 3H), 2.60 (s, 3H) |
| 256 | 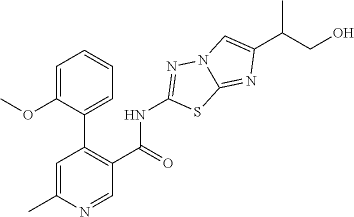 [SMILES: COc1ccccc1-c1cc(C)ncc1C(=O)Nc1nn2cc(C(C)CO)nc2s1] | (Racemic)-N-(6-(1- hydroxypropan-2-yl)- imidazo[2,1-b][1,3,4] thiadiazol-2-yl)-4-(2- methoxyphenyl)-6- methylnicotinamide | LCMS (m/z): 424 [M + H]⁺. ¹H NMR (400 MHz, DMSO- d₆) δ 8.70 (d, J = 2.6 Hz, 1H), 7.38 (d, J = 7.9 Hz, 3H), 7.32 (d, J = 2.6 Hz, 1H), 7.29-7.26 (m, 1H), 7.23 (s, 1H), 7.09- 6.97 (m, 2H), 4.33-4.21 (m, 1H), 4.01-3.96 (m, 1H), 3.63 (s, 3H), 2.82-2.77 (m, 1H), 2.55 (d, J = 1.5 Hz, 3H), 0.97 (d, J = 6.9 Hz, 3H) |

### Example 257: Synthesis of (Racemic)-4-(5-cyano-2-methoxyphenyl)-6-methyl-N-(6-(tetrahydro-2H-pyran-3-yl)thiazolo[4,5-b]pyrazin-2-yl)nicotinamide

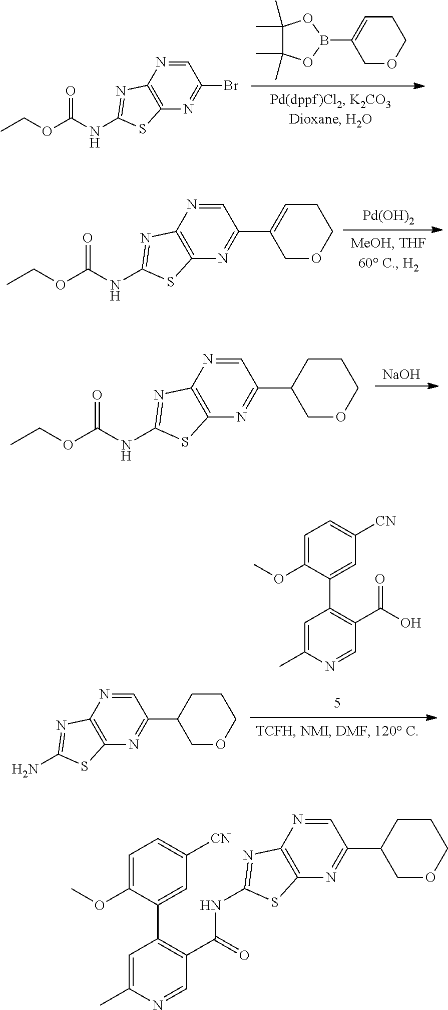

Step 1: A mixture of ethyl (6-bromothiazolo[4,5-b]pyrazin-2-yl)carbamate (600 mg, 1.9 mmol), 2-(5,6-dihydro-2H-pyran-3-yl)-4,4,5,5-tetramethyl-1,3,2-dioxaborolane (498 mg, 2.3 mmol) and K₂CO₃ (820 mg, 5.9 mmol) in 1,4-dioxane (6 mL) and H₂O (0.6 mL) was added Pd(dppf)Cl₂ (144 mg, 0.2 mmol) in one portion at 25° C. under a nitrogen atmosphere. The reaction mixture was heated at 110° C. for 12 h under a nitrogen atmosphere. The resulting mixture was cooled to 25° C. and diluted with water (20 mL). The mixture was extracted with ethyl acetate (3×30 mL). The combined organic phase was washed with brine, dried over Na₂SO₄, filtered and concentrated under reduce pressure. The residue was purified by silica gel column chromatography, eluting with DCM/MeOH (10/1) to afford ethyl (6-(5,6-dihydro-2H-pyran-3-yl)thiazolo[4,5-b]pyrazin-2-yl)carbamate (450 mg, 77% yield) as a brown solid. LCMS (ESI): [M+H]⁺=307.

Step 2: To a solution of ethyl (6-(5,6-dihydro-2H-pyran-3-yl)thiazolo[4,5-b]pyrazin-2-yl)carbamate (450 mg, 1.4 mmol) in MeOH (5 mL) and THF (5 mL) was added Pd(OH)₂ (83 mg) in one portion under a hydrogen atmosphere. The mixture was stirred at 25° C. for 18 h under hydrogen a atmosphere (1 atm.). The resulting mixture was filtered and the filter cake was washed with MeOH (2×30 mL). The combined filtrate was concentrated under reduced pressure to afford ethyl (6-(tetrahydro-2H-pyran-3-yl)thiazolo[4,5-b]pyrazin-2-yl)carbamate (410 mg, crude) as a brown solid. LCMS (ESI): [M+H]⁺=309.

Step 3: A mixture of ethyl (6-(tetrahydro-2H-pyran-3-yl)thiazolo[4,5-b]pyrazin-2-yl)carbamate (410 mg, 1.3 mmol) and NaOH (542 mg, 13.4 mmol) in MeOH (5 mL) and H₂O (5 mL) was stirred for 12 h at 80° C. The resulting mixture was cooled to 25° C. and concentrated under reduced pressure. The residue was dissolved in water (20 mL), extracted with EtOAc (2×30 mL). The combined organic layers were washed with brine, dried over Na₂SO₄, filtered and concentrated under reduce pressure. The residue was purified by reverse-phase flash chromatography (5-35% ACN/water with 0.1% formic acid modifier) to give 6-(oxan-3-yl)-[1,3]thiazolo[4,5-b]pyrazin-2-amine (125 mg, 39.7% yield) as a light brown solid. LCMS (ESI): [M+H]=237.

Step 4: A solution of 4-(5-cyano-2-methoxyphenyl)-6-methylpyridine-3-carboxylic acid (100 mg, 0.4 mmol) and TCFH (356 mg, 1.3 mmol) in ACN (10 mL) was treated with NMI (208 mg, 2.5 mmol) at 25° C. for 10 minutes followed by the addition of 6-(oxan-3-yl)-[1,3]thiazolo[4,5-b]pyrazin-2-amine (123 mg, 0.5 mmol) in portions at 25° C. The resulting mixture was stirred at 25° C. for 1 hour, then stirred at 120° C. for 4 h. The reaction mixture was cooled to 25° C. and concentrated under reduced pressure. The residue was purified by prep-HPLC (28-48% ACN/water with 0.10% formic acid modifier) to afford (Racemic)-4-(5-cyano-2-methoxyphenyl)-6-methyl-N-[6-(oxan-3-yl)-[1,3]thiazolo[4,5-b]pyrazin-2-yl]pyridine-3-carboxamide (51.9 mg, 24% yield) as a white solid. LCMS (ESI): [M+H]=487. ¹H NMR (400 MHz, Chloroform-d) δ 8.96 (s, 1H), 8.44 (s, 1H), 7.76 (d, J=8.6 Hz, 1H), 7.63 (d, J=2.1 Hz, 1H), 7.20 (s, 1H), 7.00 (d, J=8.4 Hz, 1H), 4.10-4.00 (m, 2H), 3.77 (s, 3H), 3.67 (t, J=8.7 Hz, 1H), 3.53 (t, J=8.4 Hz, 1H), 3.18 (s, 1H), 2.72 (s, 3H), 2.18-1.90 (m, 2H), 1.78 (s, 2H), 1.26 (s, 1H).

### Example 258: Synthesis of 6-(azetidin-3-yl)-4-(2-methoxyphenyl)-N-(6-(pyridin-4-yl)thiazolo[4,5-b]pyrazin-2-yl)nicotinamide

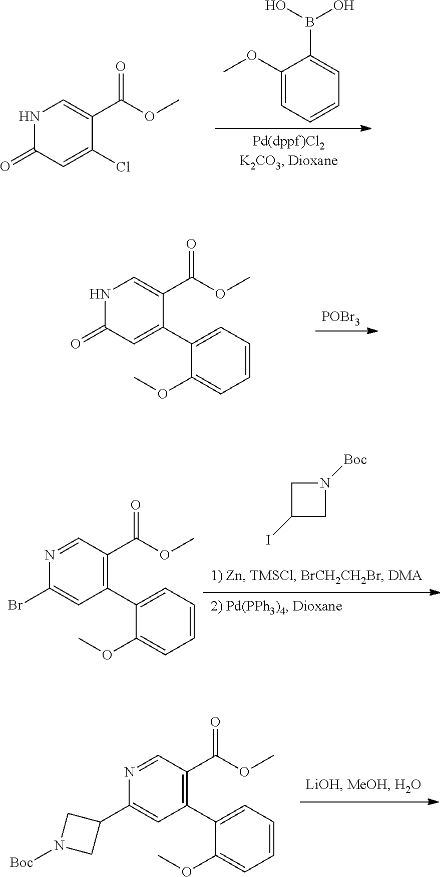

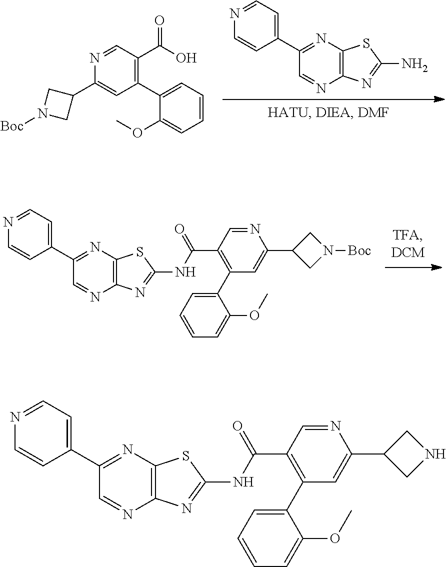 [SMILES: COc1ccccc1-c1cc(C2CN(C(=O)OC(C)(C)C)C2)ncc1C(=O)O ; Nc1nc2ncc(-c3ccncc3)nc2s1 ; COc1ccccc1-c1cc(C2CN(C(=O)OC(C)(C)C)C2)ncc1C(=O)Nc1nc2ncc(-c3ccncc3)nc2s1 ; COc1ccccc1-c1cc(C2CNC2)ncc1C(=O)Nc1nc2ncc(-c3ccncc3)nc2s1]

Step 1: To a stirred mixture of methyl 4-chloro-6-oxo-1H-pyridine-3-carboxylate (6.6 g, 35 mmol), (2-methoxyphenyl)boronic acid (6.4 g, 42.2 mmol) and K₂CO₃ (14.6 g, 106 mmol) in dioxane (100 mL) and H₂O (10 mL) was added Pd(dppf)Cl₂ (2.7 g, 3.5 mmol) in one portion at 25° C. The reaction mixture was charged with N₂ three times. The resulting mixture was heated at 120° C. for 1.5 h under a nitrogen atmosphere. The resulting mixture was concentrated under reduced pressure. The residue was purified by silica gel column chromatography, eluting with DCM/MeOH (20/1) to afford methyl 4-(2-methoxyphenyl)-6-oxo-1H-pyridine-3-carboxylate (1.6 g, 17.5% yield) as a reddish brown oil. LCMS (ESI, m/z): [M+H]=260.

Step 2: A mixture of methyl 4-(2-methoxyphenyl)-6-oxo-1H-pyridine-3-carboxylate (1.6 g, 6.2 mmol) and POBr₃ (5.3 g, 18.5 mmol) was heated at 120° C. for 1 hour under a nitrogen atmosphere. The reaction mixture was cooled to 25° C. and quenched with water/ice. The mixture was basified to pH 8 with saturated NaHCO₃ aqueous solution. The resulting mixture was extracted with EtOAc (3×50 mL). The combined organic layers were washed with brine (20 mL) and dried over anhydrous Na₂SO₄. After filtration, the filtrate was concentrated under reduced pressure. The residue was purified by silica gel column chromatography, eluting with DCM/MeOH (20/1) to afford methyl 6-bromo-4-(2-methoxyphenyl)nicotinate (1.0 g, 50.3% yield) as a grey solid. LCMS (ESI, m/z): [M+H]⁺=322, 324.

Step 3: A mixture of Zn (132 mg, 2.0 mmol) and DMA (3 mL) was stirred for 1 hour at 65° C. under a nitrogen atmosphere. TMSCl (27 mg, 0.2 mmol), 1,2-dibromoethane (47 mg, 0.2 mmol) and tert-butyl 3-iodoazetidine-1-carboxylate (439 mg, 1.6 mmol) were added dropwise to the above mixture at 65° C. The reaction mixture was stirred at 65° C. for 1 hour under a nitrogen atmosphere. The mixture was cooled to 25° C., and added to a solution of methyl 6-bromo-4-(2-methoxyphenyl)pyridine-3-carboxylate (500 mg, 1.6 mmol) and Pd(PPh₃)₄ (179 mg, 0.2 mmol) in dioxane (5 mL) at 25° C. The reaction mixture was charged with N₂ three times, and the mixture was stirred at 65° C. overnight under a nitrogen atmosphere. The reaction mixture was cooled to 25° C. and quenched with water (50 mL). The resulting mixture was extracted with EtOAc (3×50 mL). The combined organic layers were washed with brine (10 mL) and dried over anhydrous Na₂SO₄. After filtration, the filtrate was concentrated under reduced pressure. The residue was purified by prep-TLC (Pet. ether/EtOAc=1/1) to afford methyl 6-[1-(tert-butoxycarbonyl)azetidin-3-yl]-4-(2-methoxyphenyl)pyridine-3-carboxylate (480 mg, 78% yield) as a light yellow oil. LCMS (ESI, m/z): [M+H]⁺=399.

Step 4: A mixture of methyl 6-[1-(tert-butoxycarbonyl)azetidin-3-yl]-4-(2-methoxyphenyl)pyridine-3-carboxylate (450 mg, 1.1 mmol) and LiOH (2.0 M in water, 1.7 mL, 3.4 mmol) in MeOH (5 mL) was stirred for 1 hour at 60° C. under a nitrogen atmosphere. The mixture was acidified to pH 5 with 0.5M aq HCl. The resulting mixture was concentrated under reduced pressure, and the residue was purified by reverse-phase flash chromatography (10-40% ACN/water with 0.1% formic acid modifier) to afford 6-[1-(tert-butoxycarbonyl)azetidin-3-yl]-4-(2-methoxyphenyl)pyridine-3-carboxylic acid (330 mg, 76.0% yield) as an off-white solid. LCMS (ESI, m/z): [M−H]⁻=383.

Step 5: A mixture of 6-[1-(tert-butoxycarbonyl)azetidin-3-yl]-4-(2-methoxyphenyl)pyridine-3-carboxylic acid (100 mg, 0.3 mmol), HATU (148 mg, 0.4 mmol) and DIPEA (101 mg, 0.8 mmol) in DMF (3 mL) was stirred for 0.5 h at 25° C. under a nitrogen atmosphere. 6-(pyridin-4-yl)-[1,3]thiazolo[4,5-b]pyrazin-2-amine (66 mg, 0.3 mmol) was added to the above mixture, and the reaction was stirred at 120° C. for 2 h under a nitrogen atmosphere. The resulting mixture was purified directly by reverse-phase flash chromatography (10-50% ACN/water with 0.1% formic acid modifier) to afford tert-butyl 3-[4-(2-methoxyphenyl)-5-{[6-(pyridin-4-yl)-[1,3]thiazolo[4,5-b]pyrazin-2-yl]carbamoyl}pyridin-2-yl]azetidine-1-carboxylate (100 mg, 64.5% yield) as a dark yellow solid. LCMS (ESI, m/z): [M+H]⁺=596.

Step 6: A solution of tert-butyl 3-[4-(2-methoxyphenyl)-5-{[6-(pyridin-4-yl)-[1,3]thiazolo[4,5-b]pyrazin-2-yl]carbamoyl}pyridin-2-yl]azetidine-1-carboxylate (90 mg, 0.2 mmol) and TFA (1.0 mL, 13.5 mmol) in DCM (2 mL) was stirred for 1 hour at 25° C. under a nitrogen atmosphere. The resulting mixture was concentrated under reduced pressure. The residue was purified by prep-HPLC 42-59% ACN/water with 0.1 NH₄CO₃) to afford 6-(azetidin-3-yl)-4-(2-methoxyphenyl)-N-[6-(pyridin-4-yl)-[1,3]thiazolo[4,5-b]pyrazin-2-yl]pyridine-3-carboxamide (14.8 mg, 20% yield) as a yellow solid. LCMS (ESI, m/z): [M−H]-=494. ¹H NMR (300 MHz, DMSO-d₆) δ 9.13 (s, 1H), 9.01 (s, 1H), 8.71-8.59 (m, 2H), 8.13-8.00 (m, 2H), 7.42-7.20 (m, 3H), 7.10-6.93 (m, 2H), 4.28 (s, 5H), 3.52 (s, 3H).

### Example 259A & 259B: Synthesis of 4-(5-cyano-2-methoxyphenyl)-6-methyl-N-{6-[(3R or S)-oxolan-3-yl]-[1,3]thiazolo[4,5-b]pyrazin-2-yl}pyridine-3-carboxamide (259A) & 4-(5-cyano-2-methoxyphenyl)-6-methyl-N-{6-[(3S or R)-oxolan-3-yl]-[1,3]thiazolo[4,5-b]pyrazin-2-yl}pyridine-3-carboxamide (259B) with arbitrarily assigned stereochemistry

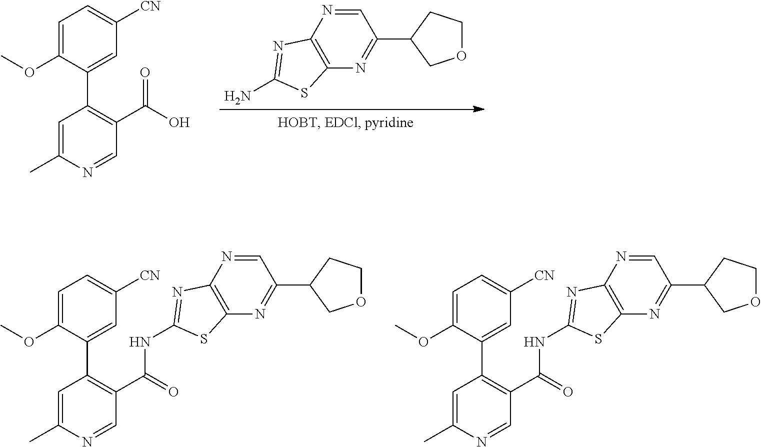

To a mixture of 4-(5-cyano-2-methoxyphenyl)-6-methylpyridine-3-carboxylic acid (150 mg, 0.6 mmol), EDCI (161 mg, 0.8 mmol) and HOBt (79 mg, 0.6 mmol) in pyridine (5 mL) was added 6-(oxolan-3-yl)-[1,3]thiazolo[4,5-b]pyrazin-2-amine (137 mg, 0.6 mmol) at 25° C. The reaction mixture was charged with N₂ three times, and the reaction was stirred for 12 h at 25° C. under a nitrogen atmosphere. The resulting mixture was diluted with water and extracted with EtOAc (3×50 mL). The combined organic layers were washed with brine (10 mL) and dried over anhydrous Na₂SO₄. After filtration, the filtrate was concentrated under reduced pressure. The residue was purified by prep-HPLC (12-30% ACN/water with 0.1% formic acid modifier) to afford a racemic product. The racemic product was separated by prep-chiral-HPLC (CHIRALPAK IC, 2*25 cm, 5 μm; 20% MeOH in 1:1 hexane:MTBE with 0.5% 2M NH₃-MeOH; flow rate 1 mL/min) to afford:

4-(5-cyano-2-methoxyphenyl)-6-methyl-N-{6-[(3R or S)-oxolan-3-yl]-[1,3]thiazolo[4,5-b]pyrazin-2-yl}pyridine-3-carboxamide (259A, 8.2 mg, 3.1% yield) as a white solid; Peak 1; RT=2.88 min. LCMS (ESI, m/z): [M+H]⁺=473. ¹H NMR (300 MHz, Methanol-d₄) δ 8.77 (s, 1H), 8.49 (s, 1H), 7.79 (d, J=9.0 Hz, 2H), 7.43 (s, 1H), 7.11 (d, J=8.4 Hz, 1H), 4.27-4.03 (m, 2H), 4.00-3.74 (m, 3H), 3.67 (s, 3H), 2.67 (s, 3H), 2.53-2.35 (m, 1H), 2.27 (m, 1H).

4-(5-cyano-2-methoxyphenyl)-6-methyl-N-{6-[(3SS or R)-oxolan-3-yl]-[1,3]thiazolo[4,5-b]pyrazin-2-yl}pyridine-3-carboxamide (259B, 9.4 mg, 4% yield) as a white solid; Peak 2; RT=3.31 min. LCMS (ESI, m/z): [M+H]=473. ¹H NMR (300 MHz, Methanol-d₄) δ 8.77 (s, 1H), 8.49 (s, 1H), 7.79 (d, J=8.9 Hz, 2H), 7.43 (s, 1H), 7.12 (d, J=8.4 Hz, 1H), 4.25-4.05 (m, 2H), 4.01-3.74 (m, 3H), 3.67 (s, 3H), 2.67 (s, 3H), 2.42 (m, 1H), 2.34-2.19 (m, 1H).

### Example 260: Synthesis of 4-(6-methoxyimidazo[1,5-a]pyridin-7-yl)-6-methyl-N-(thiazolo[4,5-d]pyrimidin-2-yl)nicotinamide

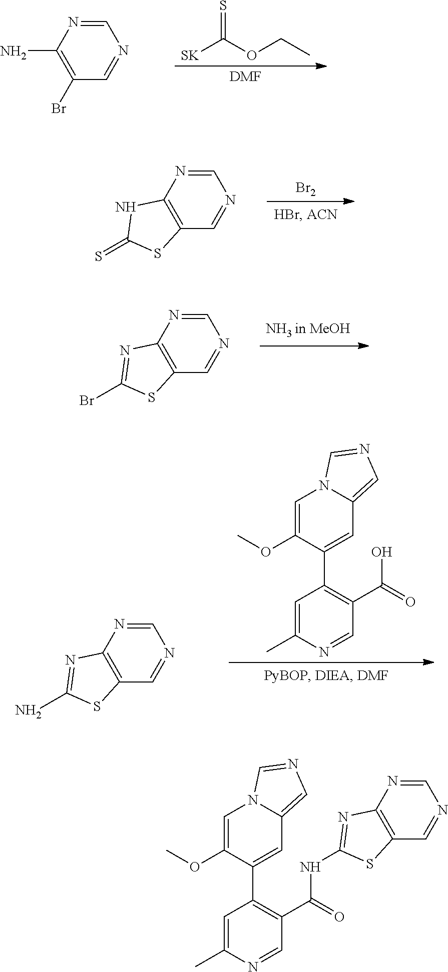

Step 1: A solution of 5-bromopyrimidin-4-amine (3.5 g, 20.1 mmol) in DMF (35 mL) was added ethoxy(potassiosulfanyl)methanethione (6.5 g, 40.2 mmol,) at 25° C. under a nitrogen atmosphere. The reaction mixture was stirred at 140° C. for 16 h under a nitrogen atmosphere. The resulting mixture was cooled to 25° C. and diluted with water (35 mL). The resulting mixture was acidified to pH 5 with HCl (4 M in H₂O). The precipitated solids were collected by filtration, washed with water (3×10 mL) and dried under reduced pressure to afford 3H-[1,3]thiazolo[4,5-d]pyrimidine-2-thione (2.9 g, crude) as a light yellow solid. LCMS (ESI): [M+H]⁺=170.

Step 2: A solution of 3H-[1,3]thiazolo[4,5-d]pyrimidine-2-thione (2.4 g, 14.2 mmol) in ACN (20 mL) was added bromine (13.6 g, 85.1 mmol) dropwise at −10° C. under a nitrogen atmosphere. The mixture was stirred for 5 minutes at −10° C. under a nitrogen atmosphere. HBr (48% in H₂O, 6.9 g, 85.1 mmol) was then added dropwise. The reaction mixture was stirred for 1 hour at −10° C., then basified to pH 8 with saturated NaHCO₃ aqueous solution and extracted with EtOAc (3×30 mL). The combined organic layers were washed with brine (3×20 mL) and dried over anhydrous Na₂SO₄. After filtration, the filtrate was concentrated under reduced pressure. The residue was purified by silica gel column chromatography, eluting with Pet. ether/EtOAc (3/1) to afford 2-bromo-[1,3]thiazolo[4,5-d]pyrimidine (800 mg, 26.1% yield) as a yellow solid. LCMS (ESI): [M+H]⁺=216, 218.

Step 3: To a sealed tube was added 2-bromo-[1,3]thiazolo[4,5-d]pyrimidine (800 mg, 3.7 mmol) and NH₃ (15 mL, 7 M in MeOH) at 25° C. The reaction mixture was stirred for 16 h at 90° C. under a nitrogen atmosphere. The resulting mixture was cooled to 25° C. and concentrated under reduced pressure. The residue was purified by prep-HPLC (10% ACN/water with 0.05% (NH₄OH) to afford [1,3]thiazolo[4,5-d]pyrimidin-2-amine (260.0 mg, 46% yield) as a yellow solid. LCMS (ESI): [M+H]=153.

Step 4: Into an 8 mL vial were added 4-{6-methoxyimidazo[1,5-a]pyridin-7-yl}-6-methylpyridine-3-carboxylic acid (284 mg, 1.0 mmol), DIPEA (389 mg, 3.0 mmol), PyBOP (783 mg, 1.5 mmol) and DMF (3 mL), and the resulting mixture was stirred for 5 minutes at 25° C. [1,3]Thiazolo[4,5-d]pyrimidin-2-amine (153 mg, 1.0 mmol) was added in one portion, and the reaction mixture was stirred for 16 h at 25° C. The reaction mixture was purified by prep-HPLC (10% ACN/water with 0.05% (NH₄OH) to afford 4-{6-methoxyimidazo[1,5-a]pyridin-7-yl}-6-methyl-N-{[1,3]thiazolo[4,5-d]pyrimidin-2-yl}pyridine-3-carboxamide (19.5 mg, 5% yield) as a light yellow solid. LCMS (ESI): [M+H]⁺=418. ¹H NMR (300 MHz, DMSO-d₆) δ 13.54 (s, 1H), 9.27 (s, 1H), 9.03 (s, 1H), 8.85 (s, 1H), 8.28 (d, J=0.8 Hz, 1H), 7.99 (s, 1H), 7.67 (s, 1H), 7.44 (s, 2H), 3.43 (s, 3H), 2.60 (s, 3H).

### Example 261: Synthesis of 4-(2-methoxyphenyl)-6-methyl-N-(6-(2-methylpyridin-4-yl)thiazolo[4,5-b]pyrazin-2-yl)nicotinamide

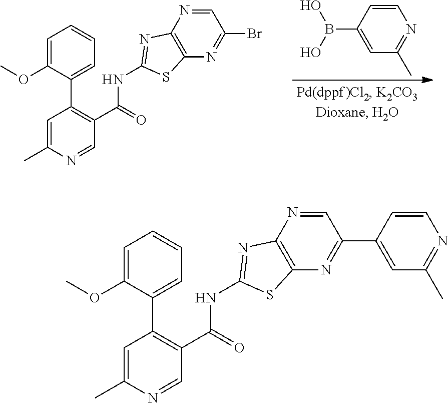

To a solution of N-{6-bromo-[1,3]thiazolo[4,5-b]pyrazin-2-yl}-4-(2-methoxyphenyl)-6-methylpyridine-3-carboxamide (37 mg, 0.1 mmol) and (2-methylpyridin-4-yl)boronic acid (15 mg, 0.1 mmol) in 1,4-dioxane (1 mL) and water (0.1 mL) was added potassium carbonate (33 mg, 0.2 mmol) and Pd(dppf)Cl₂ (11 mg, 0.01 mmol). The resulting mixture was charged with N₂ three times at room temperature, and then stirred at 90° C. for 16 h under a nitrogen atmosphere. Then the resulting mixture was cooled to room temperature and concentrated under reduced pressure. The residue was purified by reverse-phase flash chromatography (10-50% ACN/water with 0.10% formic acid modifier). The residue was purified via prep-HPLC (9-27% ACN/water with 0.1% formic acid to afford 4-(2-methoxyphenyl)-6-methyl-N-[6-(2-methylpyridin-4-yl)-[1,3]thiazolo[4,5-b]pyrazin-2-yl]pyridine-3-carboxamide (21.0 mg, 55% yield) as a yellow green solid. LCMS (ESI, m/z): [M+H]=469. ¹H NMR (400 MHz, DMSO-d₆) δ 13.41 (s, 1H), 9.32 (s, 1H), 8.78 (s, 1H), 8.60 (d, J=5.2 Hz, 1H), 8.03 (s, 1H), 7.94 (d, J=5.3 Hz, 1H), 7.42-7.34 (m, 3H), 7.11 (d, J=7.5 Hz, 1H), 6.99 (d, J=8.6 Hz, 1H), 3.52 (s, 3H), 2.59 (s, 3H), 2.58 (s, 3H).

TABLE AD
The following examples were prepared using a similar procedure to that described for Example 261

| Ex. | | | |
|---|---|---|---|
| No. | Structure | Compound Name | Characterization Data |
| 262 | 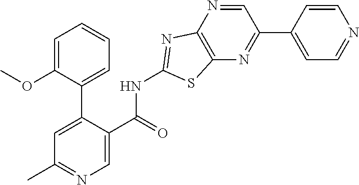 [SMILES: COc1ccccc1-c1cc(C)ncc1C(=O)Nc1nc2ncc(-c3ccncc3)nc2s1] | 4-(2-methoxyphenyl)- 6-methyl-N-(6-(pyr- idin-4-yl)thiazolo[4,5- b]pyrazin-2-yl)nicotin- amide | LCMS (m/z): 455 [M + H]⁺. ¹H NMR (300 MHz, DMSO- d₆) δ 13.42 (s, 1H), 9.34 (s, 1H), 8.90 (s, 1H), 8.71-8.69 (m, 2H), 8.60 (s, 1H), 8.11- 8.10 (m, 2H), 7.55 (s, 1H), 7.31 (s, 1H), 7.17 (s, 1H), 4.37 (s, 1H), 3.58 (s, 3H), 2.54 (s, 3H) |
| 263 | 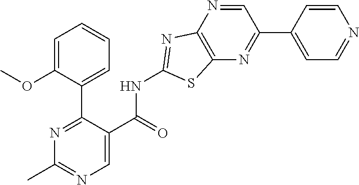 [SMILES: COc1ccccc1-c1nc(C)ncc1C(=O)Nc1nc2ncc(-c3ccncc3)nc2s1] | 4-(2-methoxyphenyl)- 2-methyl-N-(6-(pyr- idin-4-yl)thiazolo[4,5- b]pyrazin-2-yl)pyrim- idine-5-carboxamide | LCMS (m/z): 456 [M + H]⁺. ¹H NMR (400 MHz, DMSO- d₆) δ 13.58 (s, 1H), 9.38 (s, 1H), 9.01 (s, 1H), 8.80-8.68 (m, 2H), 8.18-8.08 (m, 2H), 7.70 (m, 1H), 7.54-7.46 (m, 1H), 7.16 (d, J = 7.5 Hz, 1H), 7.05-6.91 (m, 1H), 3.53 (s, 3H), 2.76 (s, 3H) |
| 264 | 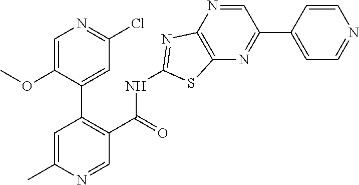 [SMILES: COc1cnc(Cl)cc1-c1cc(C)ncc1C(=O)Nc1nc2ncc(-c3ccncc3)nc2s1] | 2′-chloro-5′-methoxy- 6-methyl-N-(6-(pyr- idin-4-yl)thiazolo[4,5- b]pyrazin-2-yl)-[4,4′- bipyridine]-3-carbox- amide | LCMS (m/z): 490 [M + H]⁺. ¹H NMR (400 MHz, DMSO- d₆) δ 13.60 (s, 1H), 12.75 (s, 1H), 9.36 (s, 1H), 8.93 (s, 1H), 8.77-8.71 (m, 2H), 8.21- 8.11 (m, 4H), 7.61 (s, 1H), 7.48 (s, 1H), 3.64 (s, 3H), 2.62 (s, 3H) |
| 265 | 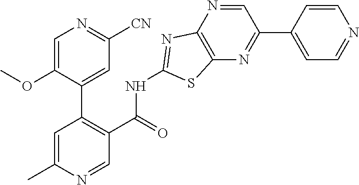 [SMILES: COc1cnc(C#N)cc1-c1cc(C)ncc1C(=O)Nc1nc2ncc(-c3ccncc3)nc2s1] | 2′-cyano-5′-methoxy- 6-methyl-N-(6-(pyr- idin-4-yl)thiazolo[4,5- b]pyrazin-2-yl)-[4,4′- bipyridine]-3-carbox- amide | LCMS (m/z): 481 [M + H]⁺. ¹H NMR (400 MHz, DMSO- d₆) δ 13.63 (s, 1H), 9.26 (s, 1H), 9.02 (s, 1H), 8.71 (d, J = 5.2 Hz, 2H), 8.56 (s, 1H), 8.12 (d, J = 5.2 Hz, 3H), 7.42 (s, 1H), 3.76 (s, 3H), 2.60 (s, 3H) |
| 266 | 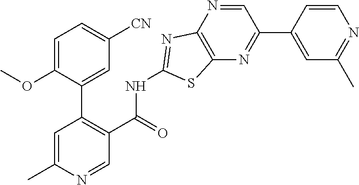 | 4-(5-cyano-2-meth- oxyphenyl)-6-methyl- N-(6-(2-methylpyri- din-4-yl)thiazolo[4,5- b]pyrazin-2-yl)nicotin-amide | LCMS (m/z): 494 [M + H]⁺. ¹H NMR (400 MHz, DMSO- d₆) δ 13.47 (s, 1H), 9.34 (s, 1H), 8.84 (s, 1H), 8.60 (d, J = 5.3 Hz, 1H), 8.03 (s, 1H), 7.93 (d, J = 5.6, 2.3 Hz, 3H), 7.45 (s, 1H), 7.19 (d, J = 8.3 Hz, 1H), 3.60 (s, 3H), 2.60- 2.49 (m, 6H) |
| 267 | 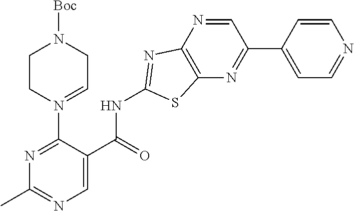 | tert-butyl 4-(2-meth- yl-5-((6-(pyridin-4- yl)thiazolo[4,5-b]pyr- azin-2-yl)carbamoyl)- pyrimidin-4-yl)piper- azine-1-carboxylate | LCMS (m/z): 534 [M + H]⁺. ¹H NMR (400 MHz, DMSO- d₆) δ 13.53 (s, 1H), 9.31 (s, 1H), 8.73 (d, J = 5.4 Hz, 2H), 8.53 (s, 1H), 8.17-8.12 (m, 2H), 3.51 (d, J = 5.5 Hz, 4H), 3.43 (d, J = 5.5 Hz, 4H), 2.47 (s, 3H), 1.39 (s, 9H) |
| 293 | 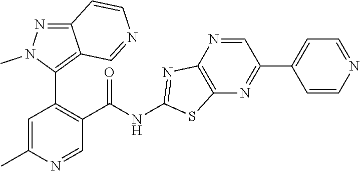 [SMILES: Cc1cc(-c2c3cnccc3nn2C)c(C(=O)Nc2nc3ncc(-c4ccncc4)nc3s2)cn1] | 6-methyl-4-(2-meth- yl-2H-pyrazolo[4,3- c]pyridin-3-yl)-N- (6-(pyridin-4-yl)thi- azolo[4,5-b]pyrazin- 2-yl)nicotinamide | LCMS (m/z): 480 [M + H]⁺. ¹H NMR (400 MHz, DMSO- d₆) δ 13.94 (s, 1H), 9.34 (s, 1H), 9.22 (s, 1H), 8.94 (s, 1H), 8.76-8.70 (m, 2H), 8.21 (d, J = 6.4 Hz, 1H), 8.16- 8.09 (m, 2H), 7.74 (s, 1H), 7.64 (d, J = 6.3 Hz, 1H), 4.08 (d, J = 1.7 Hz, 3H), 2.70 (s, 3H) |
| 294 | 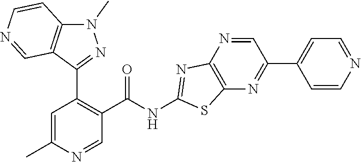 [SMILES: Cc1cc(-c2nn(C)c3ccncc23)c(C(=O)Nc2nc3ncc(-c4ccncc4)nc3s2)cn1] | 6-methyl-4-(1-meth- yl-1H-pyrazolo[4,3- c]pyridin-3-yl)-N-(6- (pyridin-4-yl)thiazolo [4,5-b]pyrazin-2-yl)- nicotinamide | LCMS (m/z): 480 [M + H]⁺. ¹H NMR (400 MHz, DMSO- d₆) δ 13.40 (s, 1H), 9.34 (s, 2H), 8.86 (s, 1H), 8.77-8.71 (m, 2H), 8.46 (d, J = 6.0 Hz, 1H), 8.18-8.12 (m, 2H), 7.92 (s, 1H), 7.74 (d, J = 6.0, 1.2 Hz, 1H), 3.95 (s, 3H), 2.69 (s, 3H) |
| 295 | 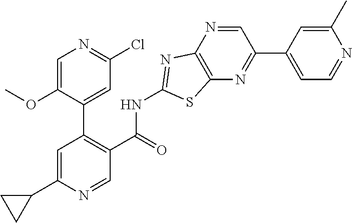 [SMILES: COc1cnc(Cl)cc1-c1cc(C2CC2)ncc1C(=O)Nc1nc2ncc(-c3ccnc(C)c3)nc2s1] | 2′-chloro-6-cyclo- propyl-5′-methoxy-N- (6-(2-methylpyridin- 4-yl)thiazolo[4,5-b] pyrazin-2-yl)-[4,4′- bipyridine]-3-carbox- amide | LCMS (m/z): 530 [M + H]⁺. ¹H NMR (400 MHz, DMSO- d₆) δ 13.50 (s, 1H), 9.34 (s, 1H), 8.85 (s, 1H), 8.60 (d, J = 5.3 Hz, 1H), 8.18 (s, 1H), 8.03 (d, J = 1.7 Hz, 1H), 7.94 (dd, J = 5.2, 1.7 Hz, 1H), 7.65 (s, 1H), 7.50 (s, 1H), 3.64 (s, 3H), 2.58 (s, 3H), 2.34-2.21 (m, 1H), 1.15- 1.03 (m, 4H) |
| 296 | 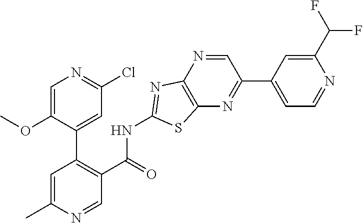 [SMILES: COc1cnc(Cl)cc1-c1cc(C)ncc1C(=O)Nc1nc2ncc(-c3ccnc(C(F)F)c3)nc2s1] | 2′-chloro-N-(6-(2- (difluoromethyl)pyri- din-4-yl)thiazolo[4,5- b]pyrazin-2-yl)-5′- methoxy-6-methyl- [4,4′-bipyridine]-3- carboxamide | LCMS (m/z): 540 [M + H]⁺. ¹H NMR (400 MHz, DMSO- d₆) δ 13.63 (s, 1H), 9.46 (s, 1H), 8.92 (s, 1H), 8.86 (d, J = 5.2 Hz, 1H), 8.44 (s, 1H), 8.35 (d, J = 5.2 Hz, 1H), 8.19 (s, 1H), 7.62 (s, 1H), 7.49 (s, 1H), 7.08 (t, J = 54.8 Hz, 1H), 3.64 (s, 3H), 2.62 (s, 3H) |
| 297 | 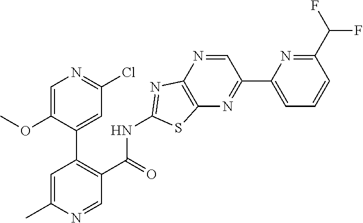 [SMILES: COc1cnc(Cl)cc1-c1cc(C)ncc1C(=O)Nc1nc2ncc(-c3cccc(C(F)F)n3)nc2s1] | 2′-chloro-N-(6-(6- (difluoromethyl)pyri- din-2-yl)thiazolo[4,5- b]pyrazin-2-yl)-5′- methoxy-6-methyl- [4,4′-bipyridine]-3- carboxamide | LCMS (m/z): 540 [M + H]⁺. ¹H NMR (400 MHz, DMSO- d₆) δ 13.59 (s, 1H) δ 9.49 (s, 1H), 8.94 (s, 1H), 8.51 (d, J = 8.1 Hz, 1H), 8.19 (d, J = 7.4 Hz, 2H), 7.79 (d, J = 7.7 Hz, 1H), 7.58 (s, 1H), 7.45 (s, 1H), 7.09 (t, J = 54.9 Hz, 1H), 3.65 (s, 3H), 2.61 (s, 3H) |

### Example 268: Synthesis of 4-(5-cyano-2-methoxyphenyl)-6-methyl-N-(6-(tetrahydro-2H-pyran-4-yl)thiazolo[4,5-b]pyrazin-2-yl)nicotinamide

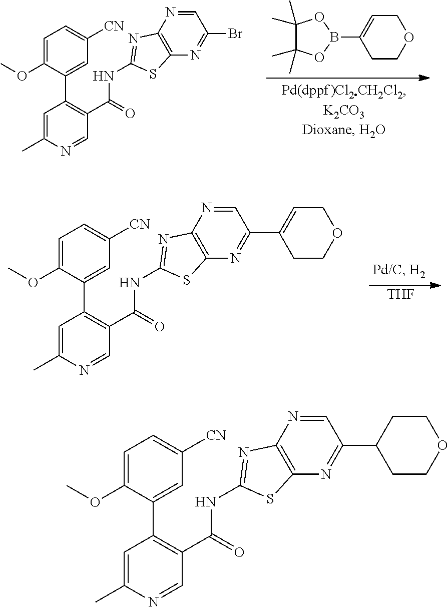 [SMILES: COc1ccc(C#N)cc1-c1cc(C)ncc1C(=O)Nc1nc2ncc(Br)nc2s1 ; CC1(C)OB(C2=CCOCC2)OC1(C)C ; COc1ccc(C#N)cc1-c1cc(C)ncc1C(=O)Nc1nc2ncc(C3=CCOCC3)nc2s1 ; COc1ccc(C#N)cc1-c1cc(C)ncc1C(=O)Nc1nc2ncc(C3CCOCC3)nc2s1 ; "THF"]

Step 1: To a solution of N-{6-bromo-[1,3]thiazolo[4,5-b]pyrazin-2-yl}-4-(5-cyano-2-methoxyphenyl)-6-methylpyridine-3-carboxamide (300 mg, 0.6 mmol) and 2-(3,6-dihydro-2H-pyran-4-yl)-4,4,5,5-tetramethyl-1,3,2-dioxaborolane (262 mg, 1.2 mmol) in 1,4-dioxane (10 mL) and H₂O (1 mL) were added K₂CO₃ (258 mg, 1.9 mmol) and Pd(dppf)Cl₂·DCM (51 mg, 0.1 mmol). The reaction mixture was charged with N₂ three times, and the reaction was stirred at 110° C. for 3 h under a nitrogen atmosphere. The resulting mixture was cooled to 25° C., diluted with water and extracted with EtOAc (3×60 mL). The combined organic layers were washed with brine (30 mL) and dried over anhydrous Na₂SO₄. After filtration, the filtrate was concentrated under reduced pressure. The residue was purified by reverse-phase flash chromatography (10-50% ACN/water with 0.1% formic acid) to afford 4-(5-cyano-2-methoxyphenyl)-N-[6-(3,6-dihydro-2H-pyran-4-yl)-[1,3]thiazolo[4,5-b]pyrazin-2-yl]-6-methylpyridine-3-carboxamide (180.0 mg, 60% yield) as a yellow solid. LCMS (ESI, m/z): [M+H]⁺=485.

Step 2: A mixture of 4-(5-cyano-2-methoxyphenyl)-N-[6-(3,6-dihydro-2H-pyran-4-yl)-[1,3]thiazolo[4,5-b]pyrazin-2-yl]-6-methylpyridine-3-carboxamide (100 mg, 0.2 mmol) and Pd/C (200 mg, 10%) in THF (20 mL) was stirred at 25° C. for 12 h under a hydrogen atmosphere. The resulting mixture was filtered and the filter cake was washed with THF (3×10 mL). The filtrate was concentrated under reduced pressure. The residue was purified by prep-HPLC (25-40% ACN/water with 0.1% formic acid) to afford 4-(5-cyano-2-methoxyphenyl)-6-methyl-N-[6-(oxan-4-yl)-[1,3]thiazolo[4,5-b]pyrazin-2-yl]pyridine-3-carboxamide (25 mg, 25% yield) as a white solid. LCMS (ESI, m/z): [M+H]=487. ¹H NMR (400 MHz, DMSO-d₆) δ 13.25 (s, 1H), 8.81 (s, 1H), 8.61 (s, 1H), 7.92-7.90 (m, 2H), 7.45 (s, 1H), 7.17 (d, J=9.2 Hz, 1H), 3.99-3.96 (m, 2H), 3.59 (s, 3H), 3.55-3.41 (m, 2H), 3.19-3.13 (m, 1H), 2.60 (s, 3H), 1.83-1.81 (m, 4H).

TABLE AQ
The following examples were prepared using a similar procedure to that described for Example 268

| Ex. No. | Structure | Compound Name | Characterization Data |
|---|---|---|---|
| 298 | 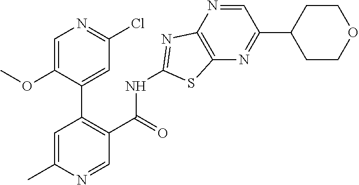 [SMILES: COc1cnc(Cl)cc1-c1cc(C)ncc1C(=O)Nc1nc2ncc(C3CCOCC3)nc2s1] | 2′-chloro-5′-methoxy- 6-methyl-N-(6-(tetra- hydro-2H-pyran-4- yl)thiazolo[4,5-b] pyrazin-2-yl)-[4,4′- bipyridine]-3-carbox- amide | LCMS (m/z): 497 [M + H]⁺. ¹H NMR (400 MHz, DMSO- d₆) δ 13.37 (s, 1H), 8.88 (s, 1H), 8.62 (s, 1H), 8.17 (s, 1H), 7.61 (s, 1H), 7.49 (s, 1H), 4.06-3.89 (m, 2H), 3.62 (s, 3H), 3.50-3.42 (m, 2H), 3.16 (d, J = 10.5 Hz, 1H), 2.61 (s, 3H), 1.83-1.81 (m, 4H) |
| 299 | 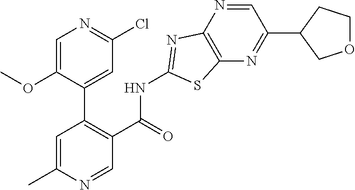 [SMILES: COc1cnc(Cl)cc1-c1cc(C)ncc1C(=O)Nc1nc2ncc(C3CCOC3)nc2s1] | 2′-chloro-5′-methoxy- 6-methyl-N-(6-(tetra- hydrofuran-3-yl)thi- azolo[4,5-b]pyrazin- 2-yl)-[4,4′-bipyri- dine]-3-carboxamide | LCMS (m/z): 483 [M + H]⁺. ¹H NMR (400 MHz, DMSO- d₆) δ 13.38 (s, 1H), 8.88 (s, 1H), 8.60 (s, 1H), 8.17 (s, 1H), 7.60 (s, 1H), 7.48 (s, 1H), 4.16-4.08 (m, 1H), 4.00-3.95 (m, 1H), 3.89- 3.70 (m, 3H), 3.62 (s, 3H), 2.61 (s, 3H), 2.40-2.30 (m, 1H), 2.24-2.13 (m, 1H) |
| 300^{%,h} | 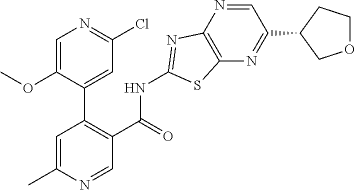 [SMILES: COc1cnc(Cl)cc1-c1cc(C)ncc1C(=O)Nc1nc2ncc([C@@H]3CCOC3)nc2s1] | (S or R)-2′-chloro- 5′-methoxy-6-meth- yl-N-(6-(tetrahydro- furan-3-yl)thiazolo [4,5-b]pyrazin-2-yl)- [4,4′-bipyridine]-3- carboxamide | LCMS (m/z): 483 [M + H]⁺. ¹H NMR (300 MHz, Meth- anol-d₄) δ 8.88 (s, 1H), 8.52 (s, 1H), 8.09 (s, 1H), 7.54 (d, J = 5.8 Hz, 2H), 4.20 (t, J = 7.9 Hz, 1H), 4.13-4.06 (m, 1H), 4.00-3.90 (m, 2H), 3.87-3.76 (m, 1H), 3.71 (s, 3H), 2.71 (s, 3H), 2.47-2.37 (m, 1H), 2.34-2.23 (m, 1H) |
| 301^{%,h} | 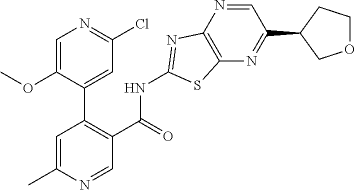 [SMILES: COc1cnc(Cl)cc1-c1cc(C)ncc1C(=O)Nc1nc2ncc([C@H]3CCOC3)nc2s1] | (R or S)-2′-chloro- 5′-methoxy-6-meth- yl-N-(6-(tetrahydro- furan-3-yl)thiazolo [4,5-b]pyrazin-2-yl)- [4,4′-bipyridine]-3- carboxamide | LCMS (m/z): 483 [M + H]⁺. ¹H NMR (300 MHz, Meth- anol-d₄) δ 8.88 (s, 1H), 8.52 (s, 1H), 8.09 (s, 1H), 7.54 (d, J = 5.9 Hz, 2H), 4.20 (t, J = 7.9 Hz, 1H), 4.13-4.06 (m, 1H), 4.00-3.90 (m, 2H), 3.86-3.75 (m, 1H), 3.71 (s, 3H), 2.71 (s, 3H), 2.50-2.37 (m, 1H), 2.35-2.24 (m, 1H) |
| 302 | 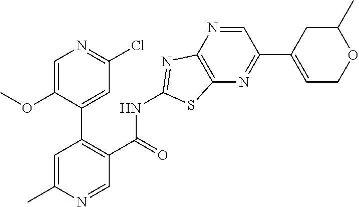 | 2′-chloro-5′-meth- oxy-6-methyl-N-(6- (2-methyl-3,6- dihydro-2H-pyran- 4-yl)thiazolo[4,5-b] pyrazin-2-yl)-[4,4′- bipyridine]-3-car- boxamide | LCMS (m/z): 509 [M + H] ⁺. ¹H NMR (300 MHz, DMSO- d₆) δ 13.51 (s, 1H), 8.97 (s, 1H), 8.70 (d, J = 7.9 Hz, 1H), 8.16 (s, 1H), 7.51 (s, 1H), 7.37 (s, 1H), 6.79 (d, J = 21.9 Hz, 1H), 4.38-4.00 (m, 2H), 3.69 (t, J = 6.4 Hz, 1H), 3.63 (s, 3H), 2.59 (s, 4H), 2.24 (t, J = 14.2 Hz, 1H), 1.27 (dd, J = 6.4, 5.1 Hz, 3H) |
| 303^{%,i} | 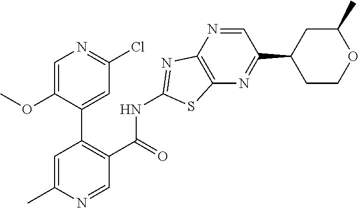 | 2′-chloro-5′-meth- oxy-6-methyl-N-(6- ((2R or S, 4R or S)- 2-methyltetrahydro- 2H-pyran-4-yl)thi- azolo[4,5-b]pyrazin- 2-yl)-[4,4′-bipyr- idine]-3-carbox- amide | LCMS (m/z): 511 [M + H]⁺. ¹H NMR (400 MHz, DMSO- d₆) δ 13.33 (s, 1H), 8.92 (s, 1H), 8.51 (s, 1H), 8.15 (s, 1H), 7.55 (s, 1H), 7.42 (s, 1H), 3.99 (dd, J = 10.8, 3.9 Hz, 1H), 3.62 (s, 3H), 3.57- 3.47 (m, 2H), 3.12 (d, J = 12.4 Hz, 1H), 2.60 (s, 3H), 1.88 (d, J = 12.5 Hz, 1H), 1.82-1.69 (m, 2H), 1.47 (q, J = 12.0 Hz, 1H), 1.15 (d, J = 6.2 Hz, 3H) |
| 304^{%,i} | 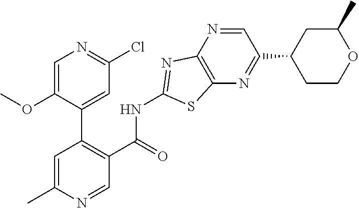 | 2′-chloro-5′-meth- oxy-6-methyl-N-(6- ((2R or S, 4S or R)- 2-methyltetrahydro- 2H-pyran-4-yl)thi- azolo[4,5-b]pyrazin- 2-yl)-[4,4′-bipyr- idine]-3-carbox- amide | LCMS (m/z): 511 [M + H]⁺. ¹H NMR (400 MHz, DMSO- d₆) δ 13.29 (s, 1H), 8.92 (s, 1H), 8.57 (s, 1H), 8.15 (s, 1H), 7.55 (d, J = 2.1 Hz, 1H), 7.42 (s, 1H), 3.95-3.83 (m, 1H), 3.79-3.71 (m, 1H), 3.66 (dd, J = 8.2, 3.7 Hz, 1H), 3.63 (s, 3H), 3.47-3.39 (m, 1H), 2.60 (s, 3H), 2.17-2.08 (m, 1H), 2.02-1.86 (m, 2H), 1.74-1.66 (m, 1H), 1.17 (d, J = 6.4 Hz, 3H) |
| 305^{%,j} | 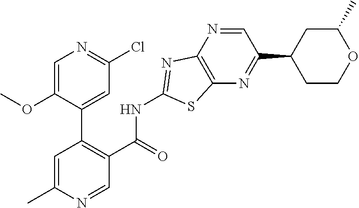 | 2′-chloro-5′-meth- oxy-6-methyl-N-(6- ((2S or R, 4R or S)- 2-methyltetrahydro- 2H-pyran-4-yl)thi- azolo[4,5-b]pyrazin- 2-yl)-[4,4′-bipyr- idine]-3-carbox- amide | LCMS (m/z): 511 [M + H]⁺. ¹H NMR (400 MHz, DMSO- d₆) δ 13.34 (s, 1H), 8.90 (s, 1H), 8.55 (s, 1H), 8.16 (s, 1H), 7.57 (s, 1H), 7.45 (s, 1H), 4.02-3.96 (m, 1H), 3.62 (s, 3H), 3.58-3.47 (m, 2H), 3.22-3.11 (m, 1H), 2.61 (s, 3H), 1.94-1.84 (m, 1H), 1.82-1.68 (m, 2H), 1.48 (q, J = 12.1 Hz, 1H), 1.15 (d, J = 6.1 Hz, 3H) |
| 306^{%,j} | 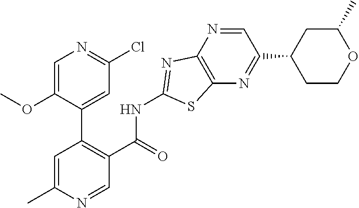 | 2′-chloro-5′-meth- oxy-6-methyl-N-(6- ((2S or R, 4S or R)- 2-methyltetrahydro- 2H-pyran-4-yl)thi- azolo[4,5-b]pyrazin- 2-yl)-[4,4′-bipyri- dine]-3-carboxamide | LCMS (m/z): 511 [M + H]⁺. ¹H NMR (400 MHz, DMSO- d₆) δ 13.34 (s, 1H), 8.92 (s, 1H), 8.58 (s, 1H), 8.16 (s, 1H), 7.55 (s, 1H), 7.42 (s, 1H), 3.92-3.84 (m, 1H), 3.80-3.72 (m, 1H), 3.62 (s, 4H), 3.43 (t, J = 5.6 Hz, 1H), 2.60 (s, 3H), 2.18-2.08 (m, 1H), 2.04-1.85 (m, 2H), 1.75-7.65 (m, 1H), 1.17 (d, J = 6.4 Hz, 3H) |

^{%}Unknown stereochemistry
^{h}Chiral separation conditions for Example 300 and 301: Prep-chiral-HPLC Opti-Chiral C9-3 column 3.0 × 50 mm, 3 μm; 1:1 EtOH: Hexane with 0.1% formic acid modifier; 1 mL/min flow rate
ⁱChiral separation conditions for Example 303 and 304: Prep-Chiral HPLC CHIRALPAK IE column, 3 25 cm, 5 μm; 30%/70% MeOH in MTBE: Hex 1:1 with 0.1% formic acid modifier; 40 mL/min flow rate; wave length: 218/326 nm to give three peaks: Peak 1 RT 12.98 min (Example 303), Peak 2 RT 21.31 min (Example 304), Peak 3 mix of Example 305 and 306
^{j}Chiral separation conditions for Example 305 and 306: Prep-Chiral HPLC CHIRALPAK IE column, 3 × 25 cm, 5 μm; 30%/70% MeOH in MTBE:Hex 1:1 with 0.1% formic acid modifier; 40 mL/min flow rate; wave length: 218/326 nm to give two peaks: Peak 1 RT 19.63 min (Example 305), Peak 2 RT 27.32 min (Example 306)

### Example 269A & 269B: Synthesis of 2′-chloro-N-(6-((1R or S,3S and R OR 1R and S,3S or R)-3-hydroxycyclopentyl)thiazolo[4,5-b]pyrazin-2-yl)-5′-methoxy-6-methyl-[4,4′-bipyridine]-3-carboxamide (269A) & 2′-chloro-N-(6-((1R and S,3S or R OR 1R or S,3S and R)-3-hydroxycyclopentyl)thiazolo[4,5-b]pyrazin-2-yl)-5′-methoxy-6-methyl-[4,4′-bipyridine]-3-carboxamide (269B) with arbitrarily assigned stereochemistry

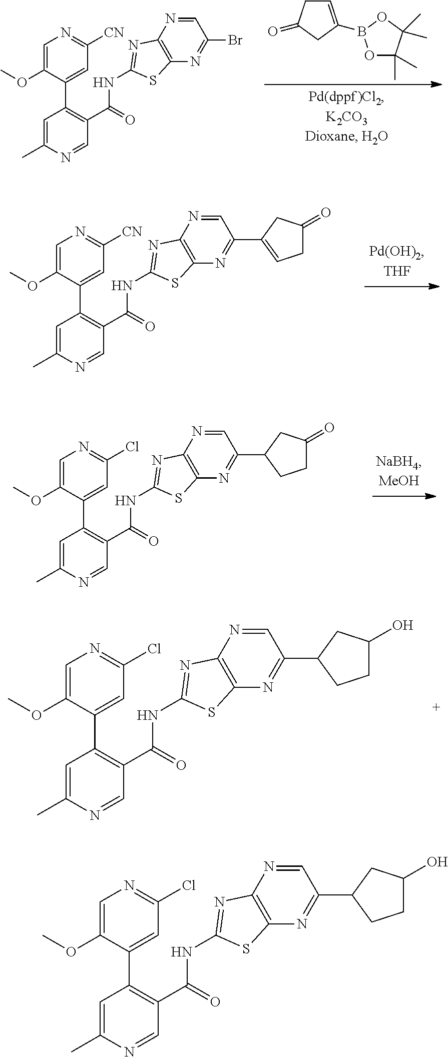 [SMILES: COc1cnc(C#N)cc1-c1cc(C)ncc1C(=O)Nc1nc2ncc(Br)nc2s1 ; CC1(C)OB(C2=CCC(=O)C2)OC1(C)C ; COc1cnc(C#N)cc1-c1cc(C)ncc1C(=O)Nc1nc2ncc(C3=CCC(=O)C3)nc2s1 ; COc1cnc(Cl)cc1-c1cc(C)ncc1C(=O)Nc1nc2ncc(C3CCC(=O)C3)nc2s1 ; COc1cnc(Cl)cc1-c1cc(C)ncc1C(=O)Nc1nc2ncc(C3CCC(O)C3)nc2s1 ; COc1cnc(Cl)cc1-c1cc(C)ncc1C(=O)Nc1nc2ncc(C3CCC(O)C3)nc2s1]

Step 1: To a stirred mixture of N-(6-bromothiazolo[4,5-b]pyrazin-2-yl)-2′-chloro-5′-methoxy-6-methyl-[4,4′-bipyridine]-3-carboxamide (200 mg, 0.4 mmol) and 3-(4,4,5,5-tetramethyl-1,3,2-dioxaborolan-2-yl)cyclopent-3-en-1-one (101 mg, 0.4 mmol) in dioxane (1 mL) and H₂O (0.1 mL) was added Pd(dppf)Cl₂ (29 mg, 0.04 mmol) in portions at 25° C. under a nitrogen atmosphere. The mixture was stirred at 80° C. for 16 h under a nitrogen atmosphere. The resulting mixture was cooled to 25° C. and concentrated under reduced pressure. The residue was purified by reverse-phase flash chromatography (10-50% ACN/water with 0.1% formic acid) to afford 2′-chloro-5′-methoxy-6-methyl-N-[6-(4-oxocyclopent-1-en-1-yl)-[1,3]thiazolo[4,5-b]pyrazin-2-yl]-[4,4′-bipyridine]-3-carboxamide (170.1 mg, 85% yield) as a white solid. LCMS (ESI, m/z): [M+H]=493.

Step 2: To a solution of 2′-chloro-5′-methoxy-6-methyl-N-[6-(4-oxocyclopent-1-en-1-yl)-[1,3]thiazolo[4,5-b]pyrazin-2-yl]-[4,4′-bipyridine]-3-carboxamide (160 mg, 0.3 mmol) in THF (2 mL) was added Pd(OH)₂ (10% wt., 4 mg) under a nitrogen atmosphere. The mixture was stirred at 25° C. for 3 h under a hydrogen atmosphere (20 atm.). After filtration, the filtrate was concentrated under reduced pressure to afford 2′-chloro-5′-methoxy-6-methyl-N-(6-(3-oxocyclopentyl)thiazolo[4,5-b]pyrazin-2-yl)-[4,4′-bipyridine]-3-carboxamide (100.2 mg, crude) as a white solid. LCMS (ESI, m/z): [M+H]⁺=495.

Step 3: A solution of 2′-chloro-5′-methoxy-6-methyl-N-[6-(3-oxocyclopentyl)-[1,3]thiazolo[4,5-b]pyrazin-2-yl]-[4,4′-bipyridine]-3-carboxamide (100 mg, 0.2 mmol) and NaBH₄ (11 mg, 0.3 mmol) in MeOH (1 mL) was stirred at 0° C. for 1 h under a nitrogen atmosphere. The resulting mixture was concentrated under reduced pressure, and the residue was purified directly by prep-HPLC (44-57% MeOH/water with 0.05% TFA) to afford 2′-chloro-N-[6-(3-hydroxycyclopentyl)-[1,3]thiazolo[4,5-b]pyrazin-2-yl]-5′-methoxy-6-methyl-[4,4′-bipyridine]-3-carboxamide (23.6 mg, 22.4% yield) as a white solid. The racemic product was purified by chiral-HPLC (Column: CHIRALPAK ID-3, 4.6*50 mm, 3 μm; 20% MeOH in 1:1 MTBE:Hex with 0.1% DEA modifier; flow rate 1.67 mL/min) to afford:

2′-chloro-N-(6-((1R or S,3S and R OR JR and S,3S or R)-3-hydroxycyclopentyl)thiazolo[4,5-b]pyrazin-2-yl)-5′-methoxy-6-methyl-[4,4′-bipyridine]-3-carboxamide (269A, 4.2 mg, 4.0%, Peak 1, RT=2.99 min). LCMS (ESI): [M+H]=497, ¹H NMR (400 MHz, DMSO-d₆) δ 13.35 (s, 1H), 8.89 (s, 1H), 8.57 (s, 1H), 8.16 (s, 1H), 7.59 (s, 1H), 7.46 (s, 1H), 4.69 (d, J=4.4 Hz, 1H), 4.26-4.22 (m, 1H), 3.62 (s, 3H), 2.61 (s, 3H), 2.40-2.27 (m, 1H), 2.10-1.48 (m, 6H).

2′-chloro-N-(6-((1R and S,3S or R OR JR or S,3S and R)-3-hydroxycyclopentyl)thiazolo[4,5-b]pyrazin-2-yl)-5′-methoxy-6-methyl-[4,4′-bipyridine]-3-carboxamide (269B, 4.0 mg, 3.9%, Peak 2, RT=4.81 min). LCMS (ESI): [M+H]⁺=497, ¹H NMR (400 MHz, DMSO-d₆) δ 13.23 (s, 1H), 8.94 (s, 1H), 8.46 (s, 1H), 8.15 (s, 1H), 7.53 (s, 1H), 7.38 (s, 1H), 4.68 (d, J=4.5 Hz, 1H), 4.25-4.21 (m, 1H), 3.62 (s, 3H), 3.29 (d, J=8.8 Hz, 1H), 2.59 (s, 3H), 2.39-2.23 (m, 1H), 1.99-1.94 (m, 2H), 1.85-1.83 (m, 1H), 1.82-1.78 (m, 2H).

### Example 270: Synthesis of (Racemic)-2′-chloro-N-(6-(5-(hydroxymethyl)tetrahydrofuran-3-yl)thiazolo[4,5-b]pyrazin-2-yl)-5′-methoxy-6-methyl-[4,4′-bipyridine]-3-carboxamide

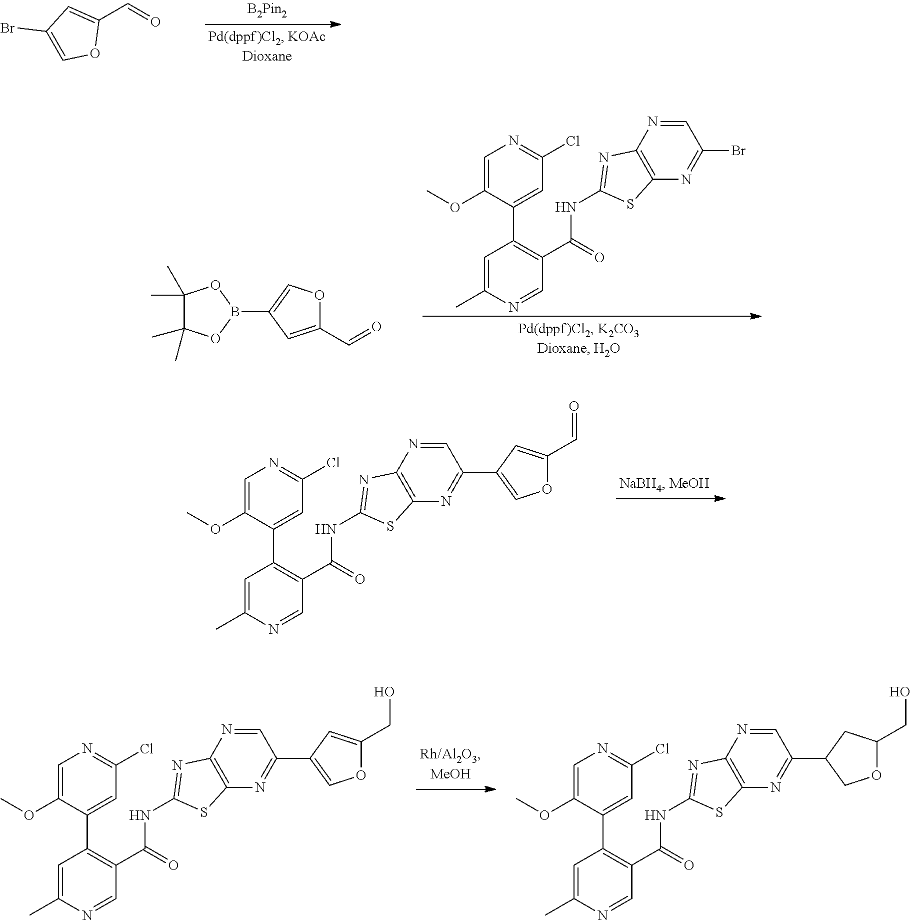 [SMILES: O=Cc1cc(Br)co1 ; CC1(C)OB(c2coc(C=O)c2)OC1(C)C ; COc1cnc(Cl)cc1-c1cc(C)ncc1C(=O)Nc1nc2ncc(Br)nc2s1 ; COc1cnc(Cl)cc1-c1cc(C)ncc1C(=O)Nc1nc2ncc(-c3coc(C=O)c3)nc2s1 ; COc1cnc(Cl)cc1-c1cc(C)ncc1C(=O)Nc1nc2ncc(-c3coc(CO)c3)nc2s1 ; COc1cnc(Cl)cc1-c1cc(C)ncc1C(=O)Nc1nc2ncc(C3COC(CO)C3)nc2s1]

Step 1: A solution of 4-bromofuran-2-carbaldehyde (200 mg, 1.1 mmol) in 1,4-dioxane (3 mL) was treated with AcOK (112 mg, 1.1 mmol) and B₂Pin₂ (1.5 g, 5.7 mmol) at 25° C. under a nitrogen atmosphere followed by the addition of Pd(dppf)Cl₂ (187 mg, 0.2 mmol) in portions at 25° C. The reaction mixture was stirred at 90° C. for 2 h under a nitrogen atmosphere. The resulting mixture was slowly cooled to 25° C. and diluted with water (5 mL). The mixture was extracted with DCM (3×20 mL) and dried over anhydrous Na₂SO₄. After filtration, the filtrate was concentrated under reduced pressure. The residue was purified by reverse-phase flash chromatography (0-60% ACN/water with 0.01M NH₄HCO₃) to afford 4-(4,4,5,5-tetramethyl-1,3,2-dioxaborolan-2-yl)furan-2-carbaldehyde (155 mg, 61% yield) as a black solid. LCMS (ESI): [M+H]⁺=223.

Step 2: A solution of N-(6-bromothiazolo[4,5-b]pyrazin-2-yl)-2′-chloro-5′-methoxy-6-methyl-[4,4′-bipyridine]-3-carboxamide (170 mg, 0.3 mmol), K₂CO₃ (143 mg, 1.0 mmol) and 4-(4,4,5,5-tetramethyl-1,3,2-dioxaborolan-2-yl)furan-2-carbaldehyde (115 mg, 0.5 mmol) in 1,4-dioxane (2 mL) and H₂O (0.2 mL) was treated with Pd(dppf)Cl₂ (50 mg, 0.1 mmol, 0.2 equiv.) in one portion at 25° C. The reaction mixture was stirred at 80° C. for 2 h under a nitrogen atmosphere. The resulting mixture was slowly cooled to 25° C. and diluted with water (10 mL). The mixture was extracted with DCM (3×20 mL). The combined organic phases were dried over anhydrous Na₂SO₄. After filtration, the filtrate was concentrated under reduced pressure. The residue was purified by reverse-phase flash chromatography (30-35% MeOH/water with 0.01 M NH₄HCO₃) to afford 2′-chloro-N-(6-(5-formylfuran-3-yl)thiazolo[4,5-b]pyrazin-2-yl)-5′-methoxy-6-methyl-[4,4′-bipyridine]-3-carboxamide (140 mg, 80% yield) as a yellow solid. LCMS (ESI): [M+H]=507.

Step 3: A solution of 2′-chloro-N-[6-(5-formylfuran-3-yl)-[1,3]thiazolo[4,5-b]pyrazin-2-yl]-5′-methoxy-6-methyl-[4,4′-bipyridine]-3-carboxamide (60 mg, 0.1 mmol) in MeOH (2 mL) was treated with NaBH₄ (9 mg, 0.2 mmol) dropwise at 0° C. for 5 minutes under a nitrogen atmosphere. The reaction mixture was stirred at 0° C. for 1.5 h under a nitrogen atmosphere.

The resulting mixture was concentrated under reduced pressure. The residue was diluted with water (10 mL), and the mixture was extracted with DCM (3×20 mL). The combined organic layers were washed with brine and dried over anhydrous Na₂SO₄. After filtration, the filtrate was concentrated under reduced pressure. The residue was purified by prep-TLC (DCM/MeOH=15/1) to afford 2′-chloro-N-(6-(5-(hydroxymethyl)furan-3-yl)thiazolo[4,5-b]pyrazin-2-yl)-5′-methoxy-6-methyl-[4,4′-bipyridine]-3-carboxamide (25 mg, 42% yield) as a yellow solid. LCMS (ESI): [M+H]⁺=509.0.

Step 4: A solution of 2′-chloro-N-(6-(5-(hydroxymethyl)furan-3-yl)thiazolo[4,5-b]pyrazin-2-yl)-5′-methoxy-6-methyl-[4,4′-bipyridine]-3-carboxamide (50 mg, 0.1 mmol) in MeOH (1 mL) was treated with Rh/Al₂O₃ (10 mg) in one portion at 25° C. The reaction mixture was stirred at 50° C. for 16 h under a hydrogen atmosphere. The resulting mixture was slowly cooled to 25° C. After filtration, the filter cake was washed with MeOH (3×20 mL). The filtrate was concentrated under reduced pressure and the residue was purified by prep-HPLC (13-33% ACN/water with 0.1% formic acid modifier) to afford (Racemic)-2′-chloro-N-(6-(5-(hydroxymethyl)tetrahydrofuran-3-yl)thiazolo[4,5-b]pyrazin-2-yl)-5′-methoxy-6-methyl-′[4,4′-bipyridine]-3-carboxamide (2.6 mg, 5.1% yield) as a white solid. LCMS (ESI): [M+H]+=513, ¹H NMR (400 MHz, DMSO-d₆) δ 13.99-12.35 (m, 1H), 8.83 (s, 1H), 8.49 (s, 1H), 8.09 (s, 1H), 7.51 (s, 1H), 7.38 (s, 1H), 4.69 (t, J=5.7 Hz, 1H), 4.04 (t, J=7.7 Hz, 1H), 4.08-3.99 (m, 1H), 3.77 (t, J=8.1 Hz, 1H), 3.81-3.70 (m, 1H), 3.55 (s, 3H), 3.43 (t, J=5.3 Hz, 2H), 2.54 (s, 3H), 2.36-2.25 (m, 1H), 2.06-1.92 (m, 1H).

### Example 271: Synthesis of 2′-chloro-N-(6-(4-hydroxypiperidin-1-yl)thiazolo[4,5-b]pyrazin-2-yl)-5′-methoxy-6-methyl-[4,4′-bipyridine]-3-carboxamide

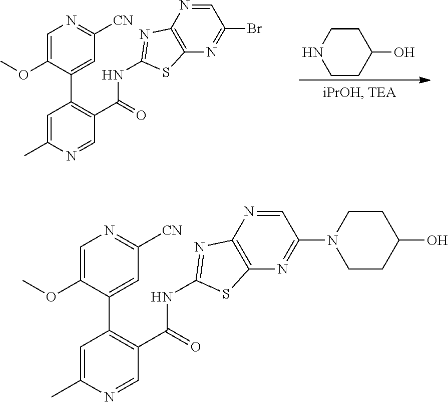 [SMILES: COc1cnc(C#N)cc1-c1cc(C)ncc1C(=O)Nc1nc2ncc(Br)nc2s1 ; OC1CCNCC1 ; COc1cnc(C#N)cc1-c1cc(C)ncc1C(=O)Nc1nc2ncc(N3CCC(O)CC3)nc2s1]

To a solution of N-(6-bromothiazolo[4,5-b]pyrazin-2-yl)-2′-chloro-5′-methoxy-6-methyl-[4,4′-bipyridine]-3-carboxamide (500 mg, 1.0 mmol) in isopropanol (20 mL) was added piperidin-4-ol (309 mg, 3.0 mmol) and TEA (617 mg, 6.1 mmol) in one portion at 25° C. The reaction mixture was charged with N₂ three times, and the mixture was stirred at 140° C. for 16 h. The resulting mixture was cooled to 25° C., and the resulting mixture was concentrated under reduced pressure. The residue was purified by reverse-phase flash chromatography (10-70% ACN/water with 0.1% formic acid) and then by prep-HPLC (12-30% ACN/water with 0.05% NH₄OH) to afford 2′-chloro-N-(6-(4-hydroxypiperidin-1-yl)thiazolo[4,5-b]pyrazin-2-yl)-5′-methoxy-6-methyl-[4,4′-bipyridine]-3-carboxamide (55.5 mg, 9.9% yield) as a yellow solid. LCMS (ESI, m/z): [M+H]⁺=512, ¹H NMR (400 MHz, DMSO-d₆) δ 12.95 (s, 1H), 8.85 (s, 1H), 8.33 (s, 1H), 8.16 (s, 1H), 7.56 (s, 1H), 7.44 (s, 1H), 4.74 (d, J=4.2 Hz, 1H), 4.03 (d, J=12.6 Hz, 2H), 3.74 (d, J=8.6 Hz, 1H), 3.63 (s, 3H), 3.23 (d, J=13.1 Hz, 2H), 2.60 (s, 3H), 1.95-1.72 (m, 2H), 1.42 (d, J=12.8 Hz, 2H).

TABLE AE
The following examples were prepared using a similar procedure to that described for
Example 271

| Ex. | | Compound | |
|---|---|---|---|
| No. | Structure | Name | Characterization Data |
| 272 | 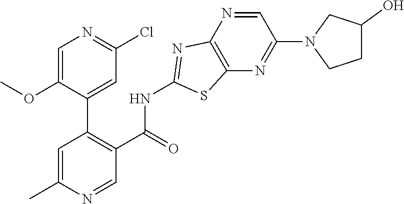 [SMILES: COc1cnc(Cl)cc1-c1cc(C)ncc1C(=O)Nc1nc2ncc(N3CCC(O)C3)nc2s1] | (Racemic)-2′- chloro-N-(6-(3- hydroxypyrrolidin- 1-yl)thiazolo[4,5- b]pyrazin-2-yl)-5′- methoxy-6- methyl-[4,4′- bipyridine]-3- carboxamide | LCMS (m/z): 498 [M + H]⁺. ¹H NMR (400 MHz, DMSO-d₆) δ 12.91 (s, 1H), 8.84 (s, 1H), 8.17 (s, 1H), 7.96 (s, 1H), 7.57 (s, 1H), 7.45 (s, 1H), 5.03 (d, J = 3.6 Hz, 1H), 4.43 (s, 1H), 3.63 (s, 3H), 3.56 (d, J = 6.9 Hz, 3H), 3.41 3.39 (m, 1H), 2.60 (s, 3H), 2.04 (t, J = 8.6 Hz, 1H), 1.93 (d, J = 3.7 Hz, 1H) |
| 273 | 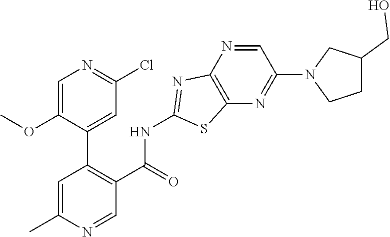 [SMILES: COc1cnc(Cl)cc1-c1cc(C)ncc1C(=O)Nc1nc2ncc(N3CCC(CO)C3)nc2s1] | (Racemic)-2′- chloro-N-(6-(3- (hydroxymethyl) pyrrolidin-1- yl)thiazolo[4,5- b]pyrazin-2-yl)-5′- methoxy-6- methyl-[4,4′- bipyridine]-3- carboxamide | LCMS (m/z): 512 [M + H]⁺. ¹H NMR (300 MHz, DMSO-d₆) δ 12.92 (s, 1H), 8.84 (s, 1H), 8.17 (s, 1H), 7.95 (s, 1H), 7.58 (s, 1H), 7.45 (s, 1H), 4.76 (t, J = 5.2 Hz, 1H), 3.63 (s, 3H), 3.62- 3.52 (m, 2H), 3.51-3.39 (m, 3H), 3.27 (t, J = 6.5 Hz, 1H), 2.60 (s, 3H), 2.47-2.38 (m, 1H), 2.08-2.04 (m, 1H), 1.79 (t, J = 7.4 Hz, 1H) |
| 307 | 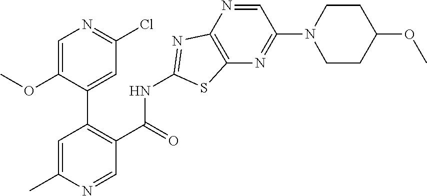 | 2′-chloro-5′- methoxy-N-(6-(4- methoxypiperidin- 1-yl)thiazolo[4,5- b]pyrazin-2-yl)-6- methyl-[4,4′- bipyridine]-3- carboxamide | LCMS (m/z): 526 [M + H]⁺. ¹H NMR (400 MHz, DMSO-d₆) δ 12.97 (s, 1H), 8.83 (s, 1H), 8.36 (s, 1H), 8.17 (s, 1H), 7.58 (s, 1H), 7.46 (s, 1H), 3.97 (d, J = 13.9 Hz, 2H), 3.62 (s, 3H), 3.50- 3.37 (m, 2H), 3.29 (s, 4H), 2.60 (s, 3H), 2.01-1.87 (m, 2H), 1.49 (d, J = 8.9 Hz, 2H) |

### Example 274: Synthesis of 6-methyl-N-(6-(pyridin-4-yl)thiazolo[4,5-b]pyrazin-2-yl)-4-(4-oxa-7-azaspiro[2.5]octan-7-yl)nicotinamide

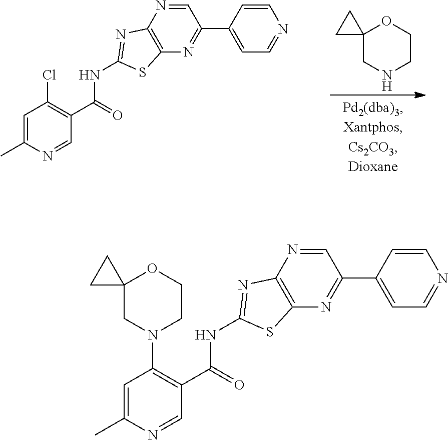 [SMILES: Cc1cc(Cl)c(C(=O)Nc2nc3ncc(-c4ccncc4)nc3s2)cn1 ; C1COC2(CC2)CN1 ; Cc1cc(N2CCOC3(CC3)C2)c(C(=O)Nc2nc3ncc(-c4ccncc4)nc3s2)cn1]

To a solution of 4-chloro-6-methyl-N-[6-(pyridin-4-yl)-[1,3]thiazolo[4,5-b]pyrazin-2-yl]pyridine-3-carboxamide (50 mg, 0.1 mmol) in 1,4-dioxane (2 mL) was added Pd₂(dba)₃ (12 mg, 0.01 mmol), Xantphos (15 mg, 0.02 mmol) and Cs₂CO₃ (128 mg, 0.4 mmol) in one portion at 25° C. The reaction mixture was charged with N₂ three times, and the reaction mixture was stirred at 120° C. for 1 hour under a nitrogen atmosphere. The resulting mixture was concentrated under reduced pressure. The residue was purified by reverse-phase flash chromatography (5-70% ACN/water with 0.1% formic acid modifier) to afford 6-methyl-4-{4-oxa-7-azaspiro[2.5]octan-7-yl}-N-[6-(pyridin-4-yl)-[1,3]thiazolo[4,5-b]pyrazin-2-yl]pyridine-3-carboxamide (13.0 mg, 22.1% yield) as a light yellow solid. LCMS (ESI, m/z): [M+H]⁺=460, ¹H NMR (300 MHz, DMSO-d₆) δ 13.45 (s, 1H), 9.35 (s, 1H), 8.92-8.57 (m, 2H), 8.50 (s, 1H), 8.30-8.07 (m, 2H), 7.02 (s, 1H), 3.77 (d, J=5.3 Hz, 2H), 3.26 (s, 4H), 2.47 (s, 3H), 0.96-0.41 (m, 4H).

### Example 275: Synthesis of 4-(6-methoxy-1-oxo-2,3-dihydroisoindol-5-yl)-6-methyl-N-[6-(pyridin-4-yl)-[1,3]thiazolo[4,5-b]pyrazin-2-yl]pyridine-3-carboxamide

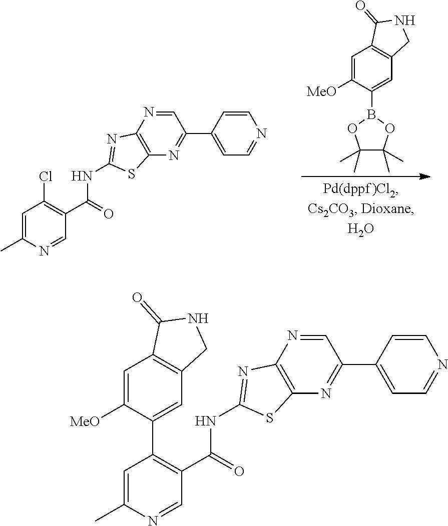 [SMILES: Cc1cc(Cl)c(C(=O)Nc2nc3ncc(-c4ccncc4)nc3s2)cn1 ; COc1cc2c(cc1B1OC(C)(C)C(C)(C)O1)CNC2=O ; COc1cc2c(cc1-c1cc(C)ncc1C(=O)Nc1nc3ncc(-c4ccncc4)nc3s1)CNC2=O]

A mixture of 6-methoxy-5-(4,4,5,5-tetramethyl-1,3,2-dioxaborolan-2-yl)-2,3-dihydroisoindol-1-one (150 mg, 0.5 mmol), 4-chloro-6-methyl-N-[6-(pyridin-4-yl)-[1,3]thiazolo[4,5-b]pyrazin-2-yl]pyridine-3-carboxamide (199 mg, 0.5 mmol), Pd(dppf)Cl₂ (76 mg, 0.1 mmol) and Cs₂CO₃ (507 mg, 1.6 mmol) in 1,4-dioxane (2 ml) and H₂O (0.2 ml) was stirred at 120° C. for 12 h under a nitrogen atmosphere. The resulting mixture was cooled to room temperature and concentrated under reduced pressure. The residue was purified by prep-HPLC (30-44% ACN/water with 0.01 M NH₄HCO₃) to afford 4-(6-methoxy-1-oxo-2,3-dihydroisoindol-5-yl)-6-methyl-N-[6-(pyridin-4-yl)-[1,3]thiazolo[4,5-b]pyrazin-2-yl]pyridine-3-carboxamide (38.1 mg, 14.4% yield) as a yellow solid. LCMS (ESI): [M+H]⁺=510, ¹H NMR (300 MHz, DMSO-d₆) δ 13.52 (s, 1H), 9.33 (s, 1H), 8.85 (s, 1H), 8.74 (d, J=5.3 Hz, 2H), 8.65 (s, 1H), 8.12-8.14 (m, 2H), 7.61 (s, 1H), 7.38 (s, 1H), 7.18 (s, 1H), 4.39 (s, 2H), 3.58 (s, 3H), 2.61 (s, 3H).

TABLE AF
The following examples were prepared using a similar procedure to that described for
Example 275

| Ex. | | | |
|---|---|---|---|
| No. | Structure | Compound Name | Characterization Data |
| 276 | 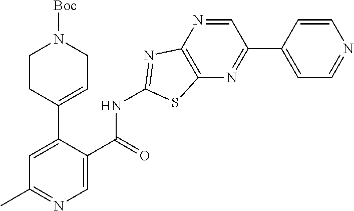 [SMILES: Cc1cc(C2=CCN(C(=O)OC(C)(C)C)CC2)c(C(=O)Nc2nc3ncc(-c4ccncc4)nc3s2)cn1] | tert-butyl 2′-methyl- 5′-((6-(pyridin-4- yl)thiazolo[4,5- b]pyrazin-2- yl)carbamoyl)-3,6- dihydro-[4,4′- bipyridine]-1 (2H)- carboxylate | LCMS (m/z): 530 [M + H]⁺. ¹H NMR (300 MHz, DMSO-d₆) δ 13.52 (s, 1H), 12.74 (s, 1H), 9.40 (s, 1H), 8.76 (s, 2H), 8.71 (s, 1H), 8.20-8.13 (m, 3H), 7.33 (s, 1H), 5.83 (s, 1H), 3.89 (d, J = 3.1 Hz, 2H), 3.50-3.44 (m, 2H), 2.55 (s, 3H), 2.37 (s, 2H), 1.34 (s, 9H) |
| 277 | 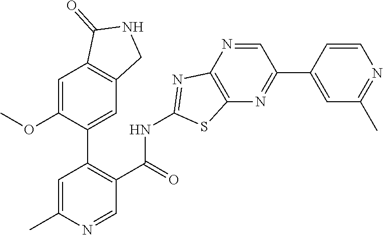 | 4-(6-methoxy-1- oxoisoindolin-5-yl)- 6-methyl-N-(6-(2- methylpyridin-4- yl)thiazolo[4,5- b]pyrazin-2- yl)nicotinamide | LCMS (m/z): 524 [M + H]⁺. ¹H NMR (400 MHz, DMSO-d₆) δ 13.50 (s, 1H), 9.26 (s, 1H), 8.87 (s, 1H), 8.63 (s, 1H), 8.58 (d, J = 5.3 Hz, 1H), 8.00 (s, 1H), 7.91 (d, J = 5.3 Hz, 1H), 7.58 (s, 1H), 7.35 (s, 1H), 7.18 (s, 1H), 4.38 (s, 2H), 3.58 (s, 3H), 2.61-2.58 (m, 6H) |
| 278 | 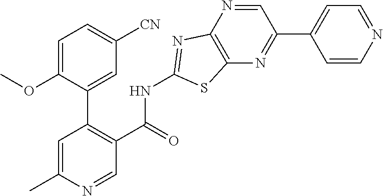 [SMILES: COc1ccc(C#N)cc1-c1cc(C)ncc1C(=O)Nc1nc2ncc(-c3ccncc3)nc2s1] | 4-(5-cyano-2- methoxyphenyl)-6- methyl-N-(6- (pyridin-4- yl)thiazolo[4,5- b]pyrazin-2- yl)nicotinamide | LCMS (m/z): 480 [M + H]⁺. ¹H NMR (400 MHz, DMSO-d₆) δ 13.50 (s, 1H), 9.35 (s, 1H), 8.86 (s, 1H), 8.77-8.71 (m, 2H), 8.17- 8.11 (m, 2H), 7.92 (d, J = 7.1 Hz, 2H), 7.44 (s, 1H), 7.18 (d, J = 8.6 Hz, 1H), 3.61 (s, 3H), 2.61 (s, 3H) |
| 279 | 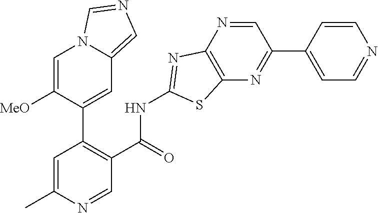 [SMILES: COc1cn2cncc2cc1-c1cc(C)ncc1C(=O)Nc1nc2ncc(-c3ccncc3)nc2s1] | 4-(6- methoxyimidazo[1,5- a]pyridin-7-yl)-6- methyl-N-(6- (pyridin-4- yl)thiazolo[4,5- b]pyrazin-2- yl)nicotinamide | LCMS (m/z): 495 [M + H]⁺. ¹H NMR (400 MHz, DMSO-d₆) δ 13.52 (s, 1H), 9.33 (s, 1H), 8.86 (s, 1H), 8.76-8.70 (m, 2H), 8.29 (s, 1H), 8.17-8.11 (m, 2H), 8.01 (s, 1H), 7.69 (s, 1H), 7.46 (s, 2H), 3.49 (s, 3H), 2.61 (s, 3H) |
| 280 | 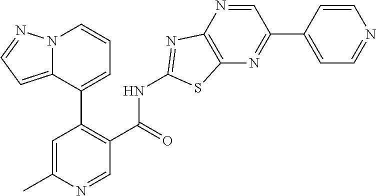 [SMILES: Cc1cc(-c2cccn3nccc23)c(C(=O)Nc2nc3ncc(-c4ccncc4)nc3s2)cn1] | 6-methyl-4- (pyrazolo[1,5- a]pyridin-4-yl)-N-(6- (pyridin-4- yl)thiazolo[4,5- b]pyrazin-2- yl)nicotinamide | LCMS (m/z): 465 [M + H]⁺. ¹H NMR (300 MHz, DMSO-d₆) δ 13.68 (s, 1H), 9.35 (s, 1H), 8.98 (s, 1H), 8.82-8.69 (m, 3H), 8.19- 8.07 (m, 2H), 7.98 (d, J = 2.4 Hz, 1H), 7.58 (s, 1H), 7.26 (d, J = 7.0, 1.0 Hz, 1H), 7.01 (t, J = 7.0 Hz, 1H), 6.42-6.40 (m, 1H), 2.65 (s, 3H) |

### Example 281: Synthesis of (Racemic)-2′-chloro-N-(6-(2,2-difluorocyclopropyl)imidazo[2,1-b][1,3,4]thiadiazol-2-yl)-5′-methoxy-6-methyl-[4,4′-bipyridine]-3-carboxamide

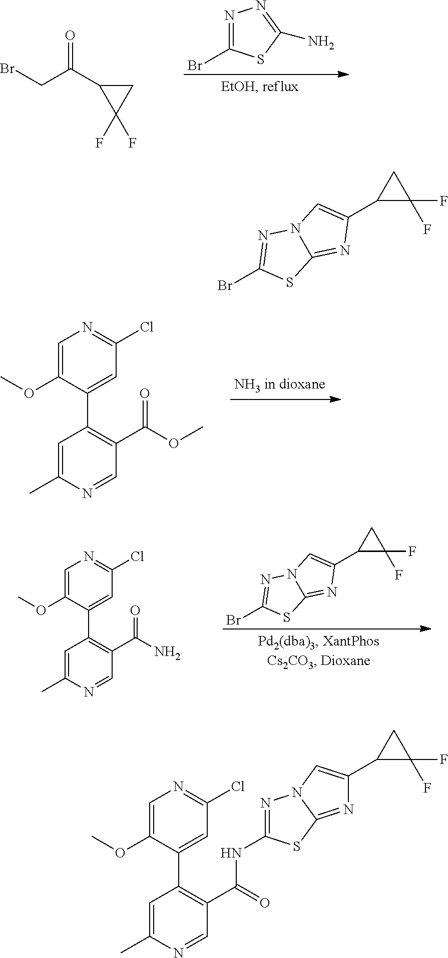

Step 1: To a stirred mixture of 2-bromo-1-(2,2-difluorocyclopropyl)ethanone (100 mg, 0.5 mmol) in EtOH (2 mL) was added 5-bromo-1,3,4-thiadiazol-2-amine (100 mg, 0.6 mmol) in one portion at 25° C. The reaction mixture was charged with N₂ three times, and the reaction mixture was stirred at 90° C. for 16 h under a nitrogen atmosphere. The resulting mixture was cooled to 25° C., and purified by reverse-phase flash chromatography (0-100% ACN/water with 0.1% formic acid modifier) to afford 2-bromo-6-(2,2-difluorocyclopropyl)imidazo[2,1-b][1,3,4]thiadiazole (17 mg, 11% yield) as a light yellow solid. LCMS (ESI): [M+H]⁺=280, 282.

Step 2: A mixture of methyl 2′-chloro-5′-methoxy-6-methyl-[4,4′-bipyridine]-3-carboxylate (200 mg, 0.7 mmol) and NH₃ in MeOH (20 mL, 7 mol/L) was stirred at 90° C. for 16 h under a nitrogen atmosphere. The resulting mixture was cooled to 25° C., then concentrated under reduced pressure. The residue was purified by reverse-phase flash chromatography (10-50% ACN/water with 0.1% formic acid) to afford 2′-chloro-5′-methoxy-6-methyl-[4,4′-bipyridine]-3-carboxamide (80 mg, 44% yield) as a white solid. LCMS (ESI): [M+H]=278.

Step 3: To a stirred mixture of 2′-chloro-5′-methoxy-6-methyl-[4,4′-bipyridine]-3-carboxamide (40 mg, 0.15 mmol) and 2-bromo-6-(2,2-difluorocyclopropyl)imidazo[2,1-b][1,3,4]thiadiazole (33 mg, 0.12 mmol) in dioxane (1 mL) were added Pd₂(dba)₃ (35 mg, 0.04 mmol), XantPhos (50 mg, 0.09 mmol) and Cs₂CO₃ (74 mg, 0.2 mmol) in one portion at 25° C. The reaction mixture was charged with N₂ three times, and stirred at 90° C. for 16 h under a nitrogen atmosphere. The resulting mixture was cooled to 25° C. and concentrated under reduced pressure. The residue was purified by prep-TLC (Pet. ether/EtOAc=1/4) to afford a crude product. The crude product was purified by reverse-phase flash chromatography (0-100% ACN/water with 0.1% formic acid modifier) to afford (Racemic)-2′-chloro-N-[6-(2,2-difluorocyclopropyl)imidazo[2,1-b][1,3,4]thiadiazol-2-yl]-5′-methoxy-6-methyl-[4,4′-bipyridine]-3-carboxamide (2.4 mg, 3.3% yield) as a white solid. LCMS (ESI): [M+H]=477, ¹H NMR (400 MHz, DMSO-d₆) δ 13.20 (s, 1H), 8.84 (s, 1H), 8.19 (s, 1H), 7.97 (d, J=3.4 Hz, 1H), 7.59 (s, 1H), 7.48 (s, 1H), 3.66 (s, 3H), 2.97-2.89 (m, 1H), 2.61 (s, 3H), 2.00-1.98 (m, 2H).

### Example 282: Synthesis of 2′-chloro-6-cyano-N-(6-cyclopropylimidazo[2,1-b][1,3,4]thiadiazol-2-yl)-5′-methoxy-[4,4′-bipyridine]-3-carboxamide

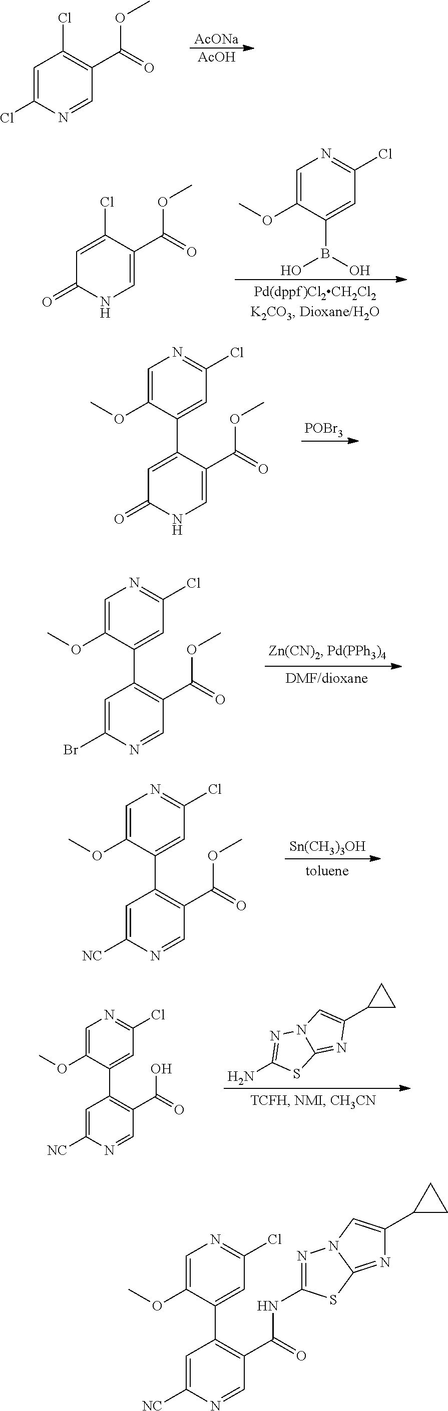

Step 1: To a stirred solution of methyl 4,6-dichloropyridine-3-carboxylate (5.0 g, 24.3 mmol) in AcOH (100 mL) was added AcONa (10.0 g, 121.3 mmol) at 25° C. The resulting mixture was stirred at 120° C. for 12 h. The reaction mixture was cooled to 25° C. and concentrated under reduced pressure. The residue was triturated with H₂O (30 mL) and basified to pH 8 with saturated NaHCO₃ aqueous solution. The precipitated solids were collected by filtration and washed with water (3×5 mL). The resulting solid was dried under reduced pressure to afford methyl 4-chloro-6-oxo-1H-pyridine-3-carboxylate (3.6 g, 79.1% yield) as a white solid. LCMS (ESI, m/z): [M+H]=188.

Step 2: To a solution of methyl 4-chloro-6-oxo-1H-pyridine-3-carboxylate (2.0 g, 10.7 mmol) and 2-chloro-5-methoxypyridin-4-ylboronic acid (2.2 g, 11.7 mmol) in 1,4-dioxane (50 mL) and H₂O (10 mL) were added K₂CO₃ (4.4 g, 32.0 mmol) and Pd(dppf)Cl₂·DCM (0.9 g, 1.1 mmol) at 25° C. The reaction mixture was charged with N₂ three times, and the mixture was stirred at 120° C. for 12 h under a nitrogen atmosphere. The reaction mixture was cooled to 25° C. and concentrated under reduced pressure. The residue was acidified to pH 7 with 2M aq HCl and extracted with DCM/MeOH (5/1, 3×100 mL). The combined organic layers were washed with brine (20 mL) and dried over anhydrous Na₂SO₄. After filtration, the filtrate was concentrated under reduced pressure. The residue was purified by silica gel column chromatography, eluting with DCM/MeOH (20/1) to afford methyl 2′-chloro-5′-methoxy-6-oxo-1H-[4,4′-bipyridine]-3-carboxylate (1.0 g, 31.8% yield) as a brown solid. LCMS (ESI, m/z): [M+H]⁺=295.

Step 3: A mixture of methyl 2′-chloro-5′-methoxy-6-oxo-1H-[4,4′-bipyridine]-3-carboxylate (1.0 g, 3.4 mmol) and phosphoryl bromide (3.9 g, 13.6 mmol) was stirred at 120° C. for 30 minutes under a nitrogen atmosphere. The reaction mixture was cooled to 25° C. and quenched with water. The mixture was basified to pH 8 with saturated NaHCO₃ aqueous solution and extracted with EtOAc (3×30 mL). The combined organic layers were washed with brine (1×10 mL) and dried over anhydrous Na₂SO₄. After filtration, the filtrate was concentrated under reduced pressure. The residue was purified by prep-TLC (Pet. ether/EtOAc=5/1) to afford methyl 6-bromo-2′-chloro-5′-methoxy-[4,4′-bipyridine]-3-carboxylate (600 mg, 49.5% yield) as a light yellow solid. LCMS (ESI, m/z): [M+H]⁺=357, 359.

Step 4: To a suspension of methyl 6-bromo-2′-chloro-5′-methoxy-[4,4′-bipyridine]-3-carboxylate (490 mg, 1.4 mmol) and Zn(CN)₂ (97 mg, 0.8 mmol) in 1,4-dioxane (4 mL) and DMF (2 mL) was added Pd(PPh₃)₄ (79 mg, 0.07 mmol) at 25° C. The reaction mixture was charged with N₂ three times at room temperature, and the reaction mixture was stirred at 90° C. for 3 h under a nitrogen atmosphere. The reaction mixture was cooled to 25° C., diluted with water and extracted with EtOAc (3×50 mL). The combined organic layers were washed with brine (10 mL) and dried over anhydrous Na₂SO₄. After filtration, the filtrate was concentrated under reduced pressure. The residue was purified by prep-TLC (Pet. ether/EtOAc=2/1) to afford methyl 2′-chloro-6-cyano-5′-methoxy-[4,4′-bipyridine]-3-carboxylate (270 mg, 65% yield) as a white solid. LCMS (ESI, m/z): [M+H]⁺=304.

Step 5: A mixture of methyl 2′-chloro-6-cyano-5′-methoxy-[4,4′-bipyridine]-3-carboxylate (150 mg, 0.5 mmol) and trimethylstannanol (179 mg, 1.0 mmol) in toluene (5 mL) was stirred at 120° C. for 6 h under a nitrogen atmosphere. The reaction mixture was cooled to 25° C. and concentrated under reduced pressure. The residue was purified by reverse-phase flash chromatography (3-50% ACN/water with 0.05% formic acid modifier) to afford 2′-chloro-6-cyano-5′-methoxy-[4,4′-bipyridine]-3-carboxylic acid (110 mg, 76.9% yield) as a white solid. LCMS (ESI, m/z): [M+H]⁺=290.

Step 6: To a stirred mixture of TCFH (58 mg, 0.2 mmol) and NMI (50 mg, 0.6 mmol) in ACN (3 mL) was added 2′-chloro-6-cyano-5′-methoxy-[4,4′-bipyridine]-3-carboxylic acid (50 mg, 0.2 mmol) and 6-cyclopropylimidazo[2,1-b][1,3,4]thiadiazol-2-amine (34 mg, 0.2 mmol). The reaction mixture was charged with N₂ three times, and the resulting mixture was stirred at 25° C. for 12 h under a nitrogen atmosphere. The reaction was diluted with water and extracted with EtOAc (3×30 mL). The combined organic layers were washed with brine (10 mL) and dried over anhydrous Na₂SO₄. After filtration, the filtrate was concentrated under reduced pressure. The residue was purified by reverse-phase flash chromatography (10-50% ACN/water with 0.05% formic acid modifier) to afford 2′-chloro-6-cyano-N-{6-cyclopropylimidazo[2,1-b][1,3,4]thiadiazol-2-yl}-5′-methoxy-[4,4′-bipyridine]-3-carboxamide (20.6 mg, 26.4% yield) as a white solid. LCMS (ESI, m/z): [M+H]⁺=452, ¹H NMR (300 MHz, DMSO-d₆) δ 13.48 (s, 1H), δ 9.15 (s, 1H), 8.26 (m, 2H), 7.75 (m, 2H), 3.66 (s, 3H), 2.09-1.88 (m, 1H), 0.86 (m, 2H), 0.74 (m, 2H).

### Example 283: Synthesis of 2′-chloro-N-(5-chloro-6-cyclopropylimidazo[2,1-b][1,3,4]thiadiazol-2-yl)-6-cyano-5′-methoxy-[4,4′-bipyridine]-3-carboxamide

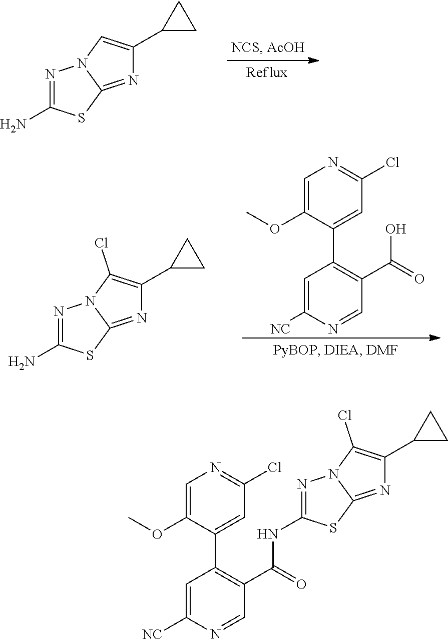

Step 1: To a solution of 6-cyclopropylimidazo[2,1-b][1,3,4]thiadiazol-2-amine (780 mg, 4.3 mmol) in AcOH (8 mL) was added NCS (578 mg, 4.3 mmol) at 25° C. The resulting mixture was stirred at 120° C. for 12 h under a nitrogen atmosphere. The reaction mixture was cooled to 25° C. and concentrated under reduced pressure. The residue was purified by reverse-phase flash chromatography (10-50% ACN/water with 0.1% formic acid) to afford 5-chloro-6-cyclopropylimidazo[2,1-b][1,3,4]thiadiazol-2-amine (420 mg, 45.2% yield) as a brown solid. LCMS (ESI, m/z): [M+H]⁺=215.

Step 2: To a stirred solution of 2′-chloro-6-cyano-5′-methoxy-[4,4′-bipyridine]-3-carboxylic acid (50 mg, 0.2 mmol) and DIPEA (67 mg, 0.5 mmol) in DMF (2 mL) was added 5-chloro-6-cyclopropylimidazo[2,1-b][1,3,4]thiadiazol-2-amine (41 mg, 0.2 mmol) in one portion at 25° C. under a nitrogen atmosphere. The reaction mixture was stirred at 25° C. for 12 h under a nitrogen atmosphere. The reaction mixture was diluted with water (20 mL) and extracted with EtOAc (3×50 mL). The combined organic layers were washed with brine (10 mL) and dried over anhydrous Na₂SO₄. After filtration, the filtrate was concentrated under reduced pressure. The residue was purified by prep-HPLC (42-59% ACN/water with 0.1% formic acid) to afford 2′-chloro-N-{5-chloro-6-cyclopropylimidazo[2,1-b][1,3,4]thiadiazol-2-yl}-6-cyano-5′-methoxy-[4,4′-bipyridine]-3-carboxamide (15.7 mg, 19% yield) as a yellow solid. LCMS (ESI, m/z): [M+H]⁺=486, ¹H NMR (400 MHz, DMSO-d₆) δ 13.70 (s, 1H), 9.13 (s, 1H), 8.29 (d, J=38.9 Hz, 2H), 7.73 (s, 1H), 3.26-3.17 (m, 3H), 2.04-1.76 (m, 1H), 1.08-0.69 (m, 4H).

### Example 284: Synthesis of 4-(2-methoxyphenyl)-6-methyl-N-(6-(pyrrolidine-1-carbonyl)imidazo[2,1-b][1,3,4]thiadiazol-2-yl)nicotinamide

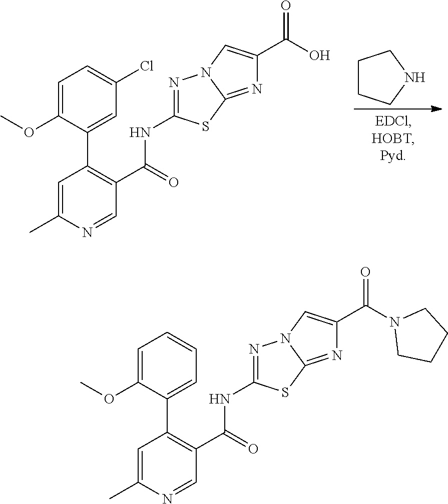

To a mixture of 2-[4-(2-methoxyphenyl)-6-methylpyridine-3-amido]imidazo[2,1-b][1,3,4]thiadiazole-6-carboxylic acid (70 mg, 0.2 mmol), EDCI (49 mg, 0.3 mmol) and HOBt (24 mg, 0.2 mmol) in pyridine (2 mL) was added pyrrolidine (18 mg, 0.3 mmol) at 25° C. The resulting mixture was stirred overnight at 25° C. under a nitrogen atmosphere. The resulting mixture was diluted with water (10 mL) and extracted with EtOAc (3×50 mL). The combined organic layers were washed with brine (30 mL) and dried over anhydrous Na₂SO₄. After filtration, the filtrate was concentrated under reduced pressure. The crude product was purified by prep-HPLC (19%-39% ACN/water with 0.1% formic acid modifier) to afford 4-(2-methoxyphenyl)-6-methyl-N-[6-(pyrrolidine-1-carbonyl)imidazo[2,1-b][1,3,4]thiadiazol-2-yl]pyridine-3-carboxamide (7.2 mg, 9.1% yield) as a white solid. LCMS (ESI, m/z): [M+H]+=463. ¹H NMR (400 MHz, DMSO-d₆) δ 8.73 (s, 1H), 8.35 (s, 1H), 7.44-7.34 (m, 2H), 7.31 (s, 1H), 7.08-7.05 (m, 1H), 7.00 (d, J=8.2 Hz, 1H), 3.94-3.92 (m, 2H), 3.55 (s, 3H), 3.47-3.45 (m, 2H), 2.57 (s, 3H), 1.88-1.85 (m, 2H), 1.81-1.78 (m, 2H).

### Example 285: Synthesis of (Cis and Trans)-2′-chloro-N-(6-(3-hydroxycyclobutyl)imidazo[2,1-b][1,3,4]thiadiazol-2-yl)-5′-methoxy-6-methyl-[4,4′-bipyridine]-3-carboxamide

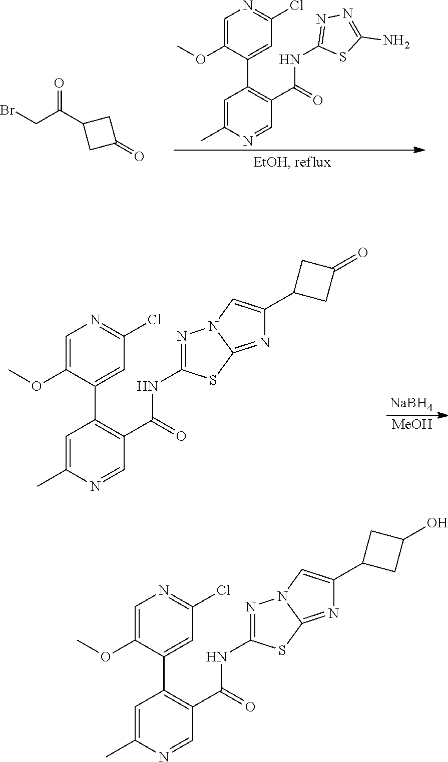 [SMILES: O=C1CC(C(=O)CBr)C1 ; "MeOH" ; COc1cnc(Cl)cc1-c1cc(C)ncc1C(=O)Nc1nnc(N)s1 ; COc1cnc(Cl)cc1-c1cc(C)ncc1C(=O)Nc1nn2cc(C3CC(=O)C3)nc2s1 ; "NaBH4" ; COc1cnc(Cl)cc1-c1cc(C)ncc1C(=O)Nc1nn2cc(C3CC(O)C3)nc2s1]

Step 1: To a solution of 3-(2-bromoacetyl)cyclobutan-1-one (400 mg, 2.0 mmol,) in ethyl alcohol (2 mL) was added N-(5-amino-1,3,4-thiadiazol-2-yl)-2′-chloro-5′-methoxy-6-methyl-[4,4′-bipyridine]-3-carboxamide (175 mg, 0.4 mmol) in one portion at 25° C. The reaction mixture was charged with N₂ three times stirred at 100° C. for 2 h under microwave irradiation. The resulting mixture was cooled to 25° cand the resulting mixture was purified by reverse-phase flash chromatography (10-50% ACN/water with 0.1% formic acid) to afford 2′-chloro-5′-methoxy-6-methyl-N-[6-(3-oxocyclobutyl)imidazo[2,1-b][1,3,4]thiadiazol-2-yl]-[4,4′-bipyridine]-3-carboxamide (156 mg, 71.5% yield) as a brown oil. LCMS (ESI, m/z): [M+H]⁺=469.

Step 2: To a solution of 2′-chloro-5′-methoxy-6-methyl-N-[6-(3-oxocyclobutyl)imidazo[2,1-b][1,3,4]thiadiazol-2-yl]-[4,4′-bipyridine]-3-carboxamide (240 mg, 0.5 mmol) in methanol (3 mL) was added NaBH₄ (29 mg, 0.7 mmol) in one portion at 25° C. The reaction mixture was stirred at 25° C. for 1 hour under a nitrogen atmosphere. The resulting mixture was concentrated under reduced pressure, then quenched with saturated aqueous NH₄Cl solution at 0° C. The mixture was extracted with ethyl acetate (3×20 mL). The combined organic phase was washed with brine (10 mL). The organic phase was dried over Na₂SO₄, filtered and concentrated under reduce pressure. Then the residue was purified by prep-HPLC (25-47% ACN/water with 0.1% formic acid modifier) to afford (Cis and Trans)-2′-chloro-N-[6-(3-hydroxycyclobutyl)imidazo[2,1-b][1,3,4]thiadiazol-2-yl]-5′-methoxy-6-methyl-[4,4′-bipyridine]-3-carboxamide (34.1 mg, 14.1% yield) as an off-white solid. LCMS (ESI, m/z): [M+H]⁺=471. ¹H NMR (300 MHz, DMSO-d₆) δ 13.14 (s, 1H), 8.84 (s, 1H), 8.16 (m, 1H), 7.78 (m, 1H), 7.57 (s, 1H), 7.46 (s, 1H), 5.04 (d, J=6.1 Hz, 1H), 4.53-4.16 (m, 1H), 3.66 (d, J=1.4 Hz, 3H), 2.60 (s, 3H), 2.35 (d, J=7.0 Hz, 2H), 2.20 (d, J=6.7 Hz, 2H), 2.08-1.85 (m, 1H).

TABLE AG
The following examples were prepared using a similar procedure to that described for
Example 285

| Ex. | | | |
|---|---|---|---|
| No. | Structure | Compound Name | Characterization Data |
| 286^{#,k} | 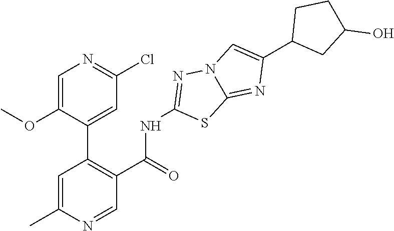 [SMILES: COc1cnc(Cl)cc1-c1cc(C)ncc1C(=O)Nc1nn2cc(C3CCC(O)C3)nc2s1] | 2′-chloro-N-(6-((1S or R,3R and S OR 1S and R,3R or S)-3- hydroxycyclopentyl) imidazo[2,1- b][1,3,4]thiadiazol-2- yl)-5′-methoxy-6- methyl-[4,4′- bipyridine]-3- carboxamide | LCMS (m/z): 485 [M + H]⁺. ¹H NMR (300 MHz, DMSO-d₆) δ 13.11 (s, 1H), 8.84 (s, 1H), 8.18 (s, 1H), 7.72 (s, 1H), 7.56 (s, 1H), 7.44 (s, 1H), 4.51-3.50 (m, 1H), 4.25-4.24 (m, 1H), 3.65 (s, 3H), 3.35-3.32 (s, 1H), 2.59 (s, 3H), 2.08-2.04 (m, 1H), 2.06-1.75 (m, 3H), 1.66-1.60 (m, 1H), 1.54-1.58 (m, 1H) |
| 287^{#,k} | 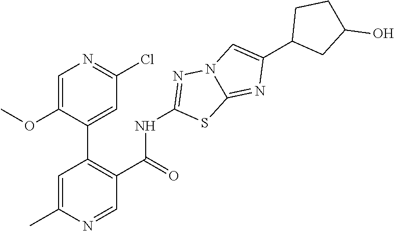 [SMILES: COc1cnc(Cl)cc1-c1cc(C)ncc1C(=O)Nc1nn2cc(C3CCC(O)C3)nc2s1] | 2′-chloro-N-(6-((1S or R,3R and S OR 1S and R,3R or S)-3- hydroxycyclopentyl) imidazo[2,1- b][1,3,4]thiadiazol-2- yl)-5′-methoxy-6- methyl-[4,4′- bipyridine]-3- carboxamide | LCMS (m/z): 485 [M + H]⁺. ¹H NMR (300 MHz, DMSO-d₆) δ 8.85 (s, 1H), 8.18 (s, 1H), 7.71 (s, 1H), 7.56 (s, 1H), 7.43 (s, 1H), 4.20-4.14 (m, 1H), 3.65 (s, 3H), 3.04-2.99 (s, 1H), 2.59 (s, 3H), 2.19-2.25 (m, 1H), 1.95-1.88 (m, 1H), 1.86- 1.72 (m, 2H), 1.63-1.55 (m, 2H) |

^{#}Unknown enantiomer
^{k}Chiral separation conditions for Example 286 and 287: Column: CHIRALPAK AD-3, 3 × 100 mm, 3 μm; Mobile Phase B: MeOH (20 mM NH₃); Flow rate: 2.00 mL/min; Peak number: 2; retention time: 3.314 min (Example 286), 3.847 min (Example 287)

### Example 288: Synthesis of (Racemic)-2′-chloro-N-(6-(3-(hydroxymethyl)cyclopentyl)imidazo[2,1-b][1,3,4]thiadiazol-2-yl)-5′-methoxy-6-methyl-[4,4′-bipyridine]-3-carboxamide

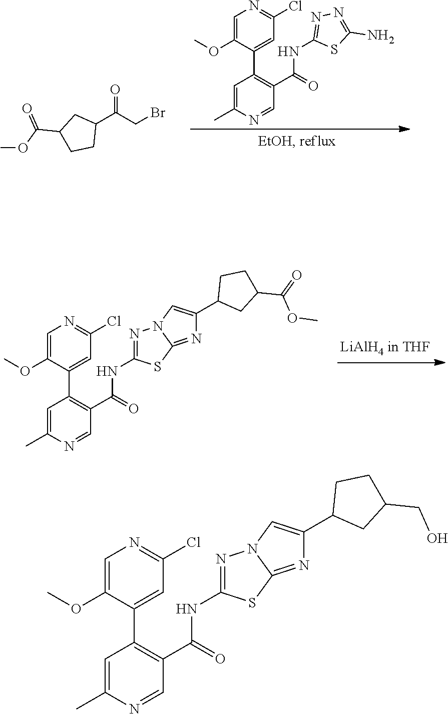

Step 1: A solution of methyl 3-(2-bromoacetyl)cyclopentane-1-carboxylate (150 mg, 0.6 mmol) and N-(5-amino-1,3,4-thiadiazol-2-yl)-2′-chloro-5′-methoxy-6-methyl-[4,4′-bipyridine]-3-carboxamide (227 mg, 0.6 mmol) in EtOH (1 mL) was stirred at 90° C. for 2 h under a nitrogen atmosphere. The reaction mixture was diluted with water (10 mL) and extracted with DCM (3×10 mL). The combined organic layers were dried over anhydrous Na₂SO₄. After filtration, the filtrate was concentrated under reduced pressure to afford methyl 3-(2-{2′-chloro-5′-methoxy-6-methyl-[4,4′-bipyridine]-3-amido}imidazo[2,1-b][1,3,4]thiadiazol-6-yl)cyclopentane-1-carboxylate (130 mg, crude) as a yellow solid. LCMS (ESI): [M+H]⁺=527.

Step 2: To a stirred solution of methyl 3-(2-{2′-chloro-5′-methoxy-6-methyl-[4,4′-bipyridine]-3-amido}imidazo[2,1-b][1,3,4]thiadiazol-6-yl)cyclopentane-1-carboxylate (120 mg, 0.2 mmol) in THF (2 mL) was added LiAlH₄ (12 mg, 0.3 mmol) at 0° C. under a nitrogen atmosphere. The mixture was stirred at 0° C. for 2 h under a nitrogen atmosphere. The reaction mixture was quenched with Na₂SO₄·10H₂O. After filtration, the filter cake was washed with DCM (3×20 mL). The combined filtrate was concentrated under reduced pressure. The residue was purified by reverse-phase flash chromatography (20-60% ACN/water with 0.1% formic acid) to afford (Racemic)-2′-chloro-N-(6-(3-(hydroxymethyl)cyclopentyl)imidazo[2,1-b][1,3,4]thiadiazol-2-yl)-5′-methoxy-6-methyl-[4,4′-bipyridine]-3-carboxamide (15.7 mg, 13.8% yield) as a white solid. LCMS (ESI): [M+H]⁺=499. ¹H NMR (400 MHz, DMSO-d₆) δ 13.10 (s, 1H), 8.84 (s, 1H), 8.18 (s, 1H), 7.72 (d, J=5.2 Hz, 1H), 7.56 (s, 1H), 7.45 (s, 1H), 4.47 (t, J=5.2 Hz, 1H), 3.65 (s, 3H), 3.08-3.04 (m, 1H), 2.59 (s, 3H), 2.40-1.90 (m, 3H), 1.87-1.61 (m, 4H), 1.53-1.16 (m, 2H).

### Example 289: Synthesis of (Racemic)-ethyl 3-(2-{2′-chloro-5′-methoxy-6-methyl-[4,4′-bipyridine]-3-amido}-[1,3]thiazolo[4,5-b]pyrazin-6-yl)cyclopentane-1-carboxylate

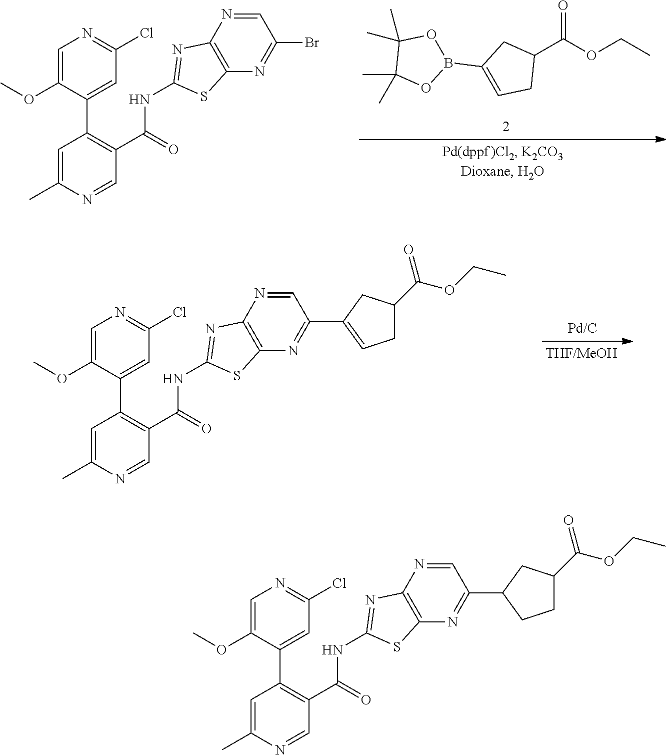

Step 1: To a mixture of N-{6-bromo-[1,3]thiazolo[4,5-b]pyrazin-2-yl}-2′-chloro-5′-methoxy-6-methyl-[4,4′-bipyridine]-3-carboxamide (200 mg, 0.4 mmol) in 1,4-dioxane (2 mL) and H₂O (0.2 mL) was added ethyl 3-(4,4,5,5-tetramethyl-1,3,2-dioxaborolan-2-yl)cyclopent-3-ene-1-carboxylate (119 mg, 0.4 mmol), Pd(dppf)Cl₂ (30 mg, 0.1 mmol) and K₂CO₃ (169 mg, 1.2 mmol) in one portion at 25° C. under a nitrogen atmosphere. The reaction mixture was stirred at 90° C. overnight under a nitrogen atmosphere. The resulting mixture was cooled to 25° C. and concentrated under reduced pressure. The residue was purified by prep-TLC (EtOAc) to afford ethyl 3-(2-{2′-chloro-5′-methoxy-6-methyl-[4,4′-bipyridine]-3-amido}-[1,3]thiazolo[4,5-b]pyrazin-6-yl)cyclopent-3-ene-1-carboxylate (160 mg, 68% yield) as an off-white solid. LCMS (ESI): [M+H]⁺=551.

Step 2: To a solution of ethyl 3-(2-{2′-chloro-5′-methoxy-6-methyl-[4,4′-bipyridine]-3-amido}-[1,3]thiazolo[4,5-b]pyrazin-6-yl)cyclopent-3-ene-1-carboxylate (160 mg, 0.3 mmol) in MeOH (1 mL) and THF (1 mL) was added Pd/C (80 mg, 10% w.t.) in a pressure tank at 25° C. The reaction mixture was stirred at 50° C. under 50 atm of hydrogen for 12 h. The resulting mixture was cooled to room temperature. After filtration, the filtercake was washed with MeOH (3×20 mL). The combined filtrate was concentrated under reduced pressure. The residue was purified by prep-HPLC (56%-69% MeOH/water with 0.05% TFA) to afford (Racemic)-ethyl 3-(2-{2′-chloro-5′-methoxy-6-methyl-[4,4′-bipyridine]-3-amido}-[1,3]thiazolo[4,5-b]pyrazin-6-yl)cyclopentane-1-carboxylate (110 mg, 65% yield) as an off-white solid. LCMS (ESI): [M+H]⁺=553. ¹H NMR (400 MHz, DMSO-d₆) δ 13.37 (s, 1H), 8.87 (s, 1H), 8.58 (s, 1H), 8.17 (s, 1H), 7.61 (s, 1H), 7.49 (s, 1H), 4.10-4.07 (m, 2H), 3.62 (s, 3H), 3.00-2.96 (m, 1H), 2.61 (s, 3H), 2.40-2.26 (m, 1H), 2.16-1.77 (m, 5H), 1.19 (t, J=7.1 Hz, 3H).

### Example 290: Synthesis of (Racemic)-2′-chloro-N-{6-[3-(hydroxymethyl)cyclopentyl]-thiazolo[4,5-b]pyrazin-2-yl}-5′-methoxy-6-methyl-[4,4′-bipyridine]-3-carboxamide

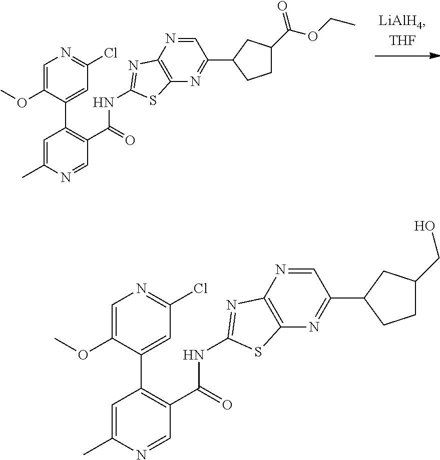

A solution of ethyl 3-(2-{2′-chloro-5′-methoxy-6-methyl-[4,4′-bipyridine]-3-amido}-[1,3]thiazolo[4,5-b]pyrazin-6-yl)cyclopentane-1-carboxylate (100 mg, 0.2 mmol) in THF (1 mL) was added LiAlH₄ (14 mg, 0.4 mmol, 2.0 equiv.) dropwise at 0° C. under a nitrogen atmosphere. The mixture was stirred at 0° C. for 30 minutes under a nitrogen atmosphere. The resulting mixture was quenched with Na₂SO₄·10 H₂O at 0° C. After filtration, the filtercake was washed with MeOH (3×20 mL). The combined filtrate was concentrated under reduced pressure. The residue was purified by prep-HPLC (10-25% ACN/water with 0.01% NH₄HCO₃ to afford (Racemic)-2′-chloro-N-{6-[3-(hydroxymethyl)cyclopentyl]-[1,3]thiazolo[4,5-b]pyrazin-2-yl}-5′-methoxy-6-methyl-[4,4′-bipyridine]-3-carboxamide (3.5 mg, 3.7% yield) as a light yellow solid. LCMS (ESI): [M+H]⁺=511. ¹H NMR (400 MHz, DMSO-d₆) δ 13.32 (s, 1H), 8.96 (s, 1H), 8.38 (s, 1H), 8.14 (s, 1H), 7.49-7.35 (s, 1H), 4.52 (t, J=5.2 Hz, 1H), 3.62 (s, 3H), 3.39 (d, J=6.0 Hz, 2H), 2.58 (s, 3H), 2.24-2.06 (m, 2H), 2.05-1.94 (m, 2H), 1.85-1.70 (m, 2H), 1.62-1.43 (m, 2H).

### Example 308A & 308B: Synthesis of 2′-chloro-N-(6-((1R or S,3S and R)—3-(hydroxymethyl)cyclopentyl)thiazolo[4,5-b]pyrazin-2-yl)-5′-methoxy-6-methyl-[4,4′-bipyridine]-3-carboxamide (308A) & 2′-chloro-N-(6-((1R or S,3R and S)-3-(hydroxymethyl)cyclopentyl)thiazolo[4,5-b]pyrazin-2-yl)-5′-methoxy-6-methyl-[4,4′-bipyridine]-3-carboxamide (308B) as a mixture of cis or trans isomers

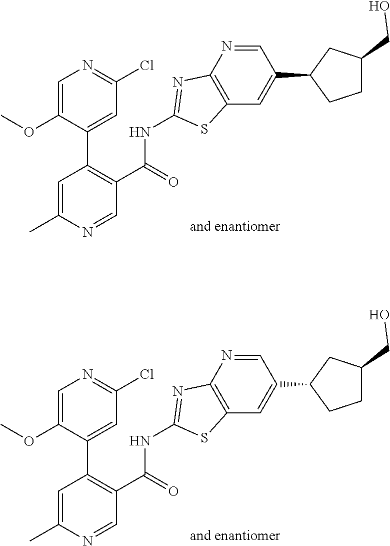 [SMILES: COc1cnc(Cl)cc1-c1cc(C)ncc1C(=O)Nc1nc2ncc([C@@H]3CC[C@H](CO)C3)cc2s1 ; COc1cnc(Cl)cc1-c1cc(C)ncc1C(=O)Nc1nc2ncc([C@H]3CC[C@H](CO)C3)cc2s1]

The racemate of example 290 was separated via prep-chiral-HPLC CHIRAL ART Amylose-SA column, 2×25 cm, 5 μm; 10%/90% MeOH/MTBE:Hex 1:1 with 0.1% TFA modifier; 20 mL/min flow rate; wave length: 218/312 nm to afford two peaks:

Peak 1 RT=14.22 min 2′-chloro-N-(6-((1R or S,3S and R)-3-(hydroxymethyl)cyclopentyl)thiazolo[4,5-b]pyrazin-2-yl)-5′-methoxy-6-methyl-[4,4′-bipyridine]-3-carboxamide (11.5 mg, 8% yield) as a white solid. LCMS (ESI, m/z): 511 [M+H]⁺. ¹H NMR (300 MHz, DMSO-d₆) δ 13.36 (s, 1H), 8.90 (s, 1H), 8.52 (s, 1H), 8.16 (s, 1H), 7.58 (s, 1H), 7.46 (s, 1H), 4.54 (t, J=5.2 Hz, 1H), 3.62 (s, 3H), 3.41-3.38 (m, 2H), 2.61 (s, 3H), 2.28-2.02 (m, 3H), 1.98-1.78 (m, 3H), 1.69-1.54 (m, 2H)

Peak 2 RT=20.02 min 2′-chloro-N-(6-((1R or S,3R and S)-3-(hydroxymethyl)cyclopentyl)thiazolo[4,5-b]pyrazin-2-yl)-5′-methoxy-6-methyl-[4,4′-bipyridine]-3-carboxamide (10.4 mg, 7% yield) as a white solid. LCMS (ESI, m/z): 511 [M+H]⁺. ¹H NMR (300 MHz, DMSO-d₆) δ 13.37 (s, 1H), 8.90 (s, 1H), 8.52 (s, 1H), 8.16 (s, 1H), 7.58 (s, 1H), 7.45 (s, 1H), 4.55 (t, J=5.2 Hz, 1H), 3.62 (s, 3H), 3.41-3.38 (m, 2H), 2.61 (s, 3H), 2.30-2.01 (m, 3H), 1.91-1.73 (m, 3H), 1.65-1.47 (m, 2H).

TABLE AR
The following examples were prepared using a similar procedure to that described for
Example 289 and 290

| Ex. | | | |
|---|---|---|---|
| No. | Structure | Compound Name | Characterization Data |
| 309^{%,l} | 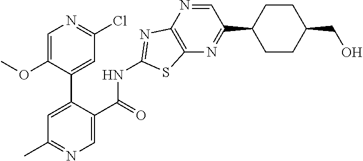 [SMILES: COc1cnc(Cl)cc1-c1cc(C)ncc1C(=O)Nc1nc2ncc([C@H]3CC[C@@H](CO)CC3)nc2s1] | (cis or trans)-2′- chloro-N-(6-(4- (hydroxymethyl)cyclo- hexyl)thiazolo[4,5- b]pyrazin-2-yl)-5′- methoxy-6-methyl- [4,4′-bipyridine]-3- carboxamide | LCMS (m/z): 525 [M + H]⁺. ¹H NMR (300 MHz, DMSO-d₆) δ 13.35 (s, 1H), 8.90 (s, 1H), 8.56 (s, 1H), 8.17 (s, 1H), 7.59 (s, 1H), 7.46 (s, 1H), 4.43 (t, J = 5.3 Hz, 1H), 3.63 (s, 3H), 3.46-3.42 (m, 2H), 2.95 (d, J = 9.7 Hz, 1H), 2.61 (s, 3H), 1.91-1.81 (m, 2H), 1.70-1.53 (m, 7H) |
| 310^{%,l} | 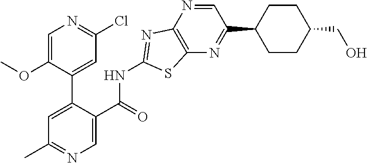 [SMILES: COc1cnc(Cl)cc1-c1cc(C)ncc1C(=O)Nc1nc2ncc([C@H]3CC[C@H](CO)CC3)nc2s1] | (trans or cis)-2′- chloro-N-(6-(4- (hydroxymethyl)cyclo- hexyl)thiazolo[4,5- b]pyrazin-2-yl)-5′- methoxy-6-methyl- [4,4′-bipyridine]-3- carboxamide | LCMS (m/z): 525 [M + H]⁺. ¹H NMR (300 MHz, DMSO-d₆) δ 8.90 (s, 1H), 8.48 (s, 1H), 8.14 (s, 1H), 7.55 (s, 1H), 7.41 (s, 1H), 3.69 (s, 3H), 3.28 (s, 2H), 2.83- 2.80 (m, 1H), 2.61 (s, 3H), 1.94- 1.86 (m, 4H), 1.63-1.54 (m, 2H), 1.53-1.45 (m, 1H), 1.29-1.23 (m, 2H), 1.12-1.03 (m, 2H) |
| 311 | 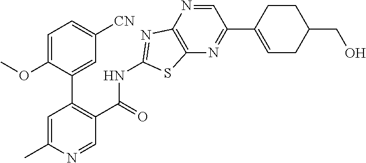 [SMILES: COc1ccc(C#N)cc1-c1cc(C)ncc1C(=O)Nc1nc2ncc(C3=CCC(CO)CC3)nc2s1] | 4-(5-cyano-2- methoxyphenyl)-N- (6-(4- (hydroxymethyl)cyclo- hex-1-en-1- yl)thiazolo[4,5- b]pyrazin-2-yl)-6- methylnicotinamide | LCMS (m/z): 513 [M + H]⁺. ¹H NMR (400 MHz, DMSO-d₆) δ 13.27 (s, 1H), 8.81 (d, J = 2.4 Hz, 2H), 7.98-7.86 (m, 2H), 7.44 (s, 1H), 7.21-7.13 (m, 1H), 6.84 (s, 1H), 4.55 (t, J = 5.2 Hz, 1H), 3.60 (s, 3H), 3.44-3.36 (m, 2H), 2.70 (d, J = 17.3 Hz, 1H), 2.60 (s, 3H), 2.39 (dd, J = 31.9, 14.1 Hz, 2H), 2.02-1.85 (m, 2H), 1.71 (s, 1H). 1.42-1.27 (m, 1H) |
| 312^{%,m} | 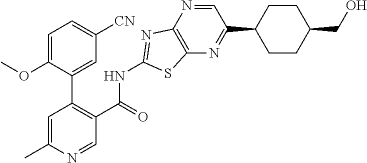 [SMILES: COc1ccc(C#N)cc1-c1cc(C)ncc1C(=O)Nc1nc2ncc([C@H]3CC[C@@H](CO)CC3)nc2s1] | (cis or trans)-4-(5- cyano-2- methoxyphenyl)-N- (6-(4- (hydroxymethyl)cyclo- hexyl)thiazolo[4,5- b]pyrazin-2-yl)-6- methylnicotinamide | LCMS (m/z): 515 [M + H]⁺. ¹H NMR (400 MHz, DMSO-d₆) δ 13.16 (s, 1H), 8.85 (s, 1H), 8.48 (s, 1H), 8.01-7.74 (m, 2H), 7.38 (s, 1H), 7.17 (s, 1H), 4.50-4.37 (m, 1H), 4.00 (s, 1H), 3.72-3.51 (m, 3H), 2.79 (s, 2H), 2.59 (s, 3H), 1.90 (s, 3H), 1.77-1.38 (m, 2H), 1.38-0.74 (m, 4H) |
| 313^{%,m} | 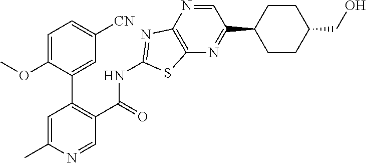 [SMILES: COc1ccc(C#N)cc1-c1cc(C)ncc1C(=O)Nc1nc2ncc([C@H]3CC[C@H](CO)CC3)nc2s1] | (trans or cis)-4-(5- cyano-2- methoxyphenyl)-N- (6-(4- (hydroxymethyl)cyclo- hexyl)thiazolo[4,5- b]pyrazin-2-yl)-6- methylnicotinamide | LCMS (m/z): 515 [M + H]⁺. ¹H NMR (400 MHz, DMSO-d₆) δ 13.23 (s, 1H), 8.81 (s, 1H), 8.57 (s, 1H), 7.97-7.86 (m, 2H), 7.44 (s, 1H), 7.21-7.12 (m, 1H), 4.43 (t, J = 5.3 Hz, 1H), 3.59 (s, 3H), 3.49- 3.40 (m, 1H), 2.95 (d, J = 9.8 Hz, 1H), 2.60 (s, 3H), 1.85 (q, J = 10.3, 9.6 Hz, 2H), 1.77-1.52 (m, 7H) |
| 314 | 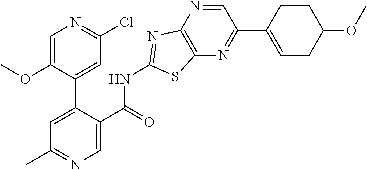 | 2′-chloro-5′- methoxy-N-[6-(4- methoxycyclohex-1- en-1-yl)[- [1,3]thiazolo[4,5- b]pyrazin-2-yl]-6- methyl-[4,4′- bipyridine]-3- carboxamide | LCMS (m/z): 523 [M + H]⁺. ¹H NMR (400 MHz, DMSO-d₆) δ 13.39 (s, 1H), 8.88 (s, 1H), 8.83 (s, 1H), 8.17 (s, 1H), 7.60 (s, 1H), 7.48 (s, 1H), 6.72 (t, J = 3.9 Hz, 1H), 3.63 (s, 3H), 3.55 (d, J = 6.0 Hz, 1H), 3.30 (s, 3H), 2.68 (d, J = 17.8 Hz, 1H), 2.61 (s, 5H), 2.27- 2.17 (m, 1H), 2.04-1.95 (m, 1H), 1.79-1.66 (m, 1H) |

^{%}Unknown stereochemistry
^{l}Chiral separation conditions for Example 309 and 310: chiral-HPLC Lux 5 um Cellulose-4 column 3 × 25 cm, 5 μm; 40%/60% EtOH/Hexane with 0.1% formic acid; 40 mL/min flow rate; wave length: 218/328 nm, Peak 1 RT 16.68 min (Example 309), Peak 2 RT 22.99 min (Example 310)
^{m}Chiral separation conditions for Example 312 and 313: Prep-chiral HPLC CHIRAL ART Amylose-C NEO column, 3 × 25 cm, 10 μm; 50%/50% EtOH/hexane with 0.1% formic acid modifier; 20 mL/min flow rate; wave length: 214/238 nm to give two peaks: Peak 1 RT 8.8125 (Example 312), Peak 2 RT 12.4135 (Example 313)

### Example 315: Synthesis of 6-(3-Amino-5-chlorophenoxy)-2′-chloro-N-(6-cyclopropylimidazo[2,1-b][1,3,4]thiadiazol-2-yl)-5′-methoxy-[4,4′-bipyridine]-3-carboxamide

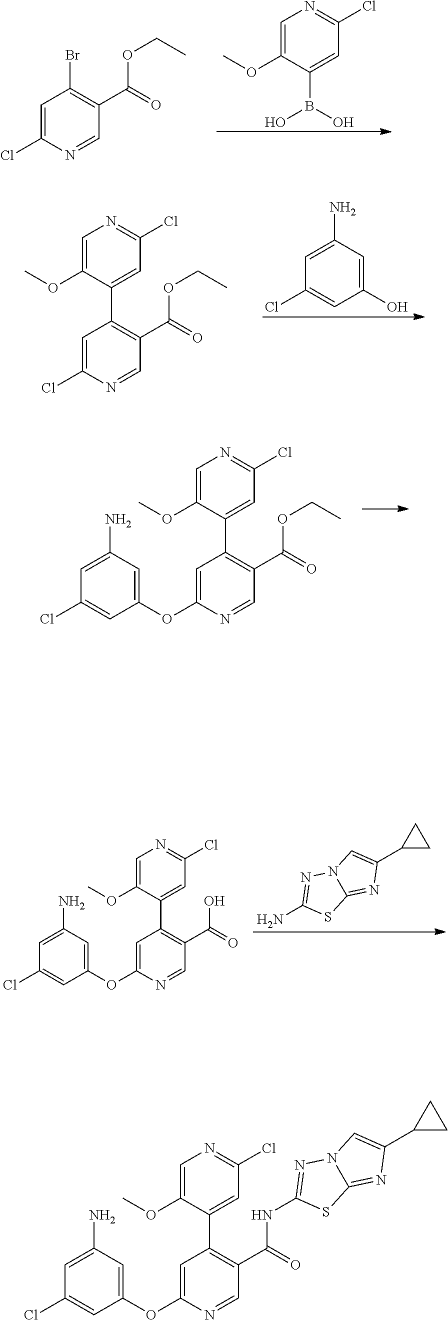

Step 1: To a solution of ethyl 4-bromo-6-chloronicotinate (1.00 g, 3.78 mmol) in 1,4-dioxane (25 mL) and water (5 mL) was added 1,1′-bis(di-t-butylphosphino)ferrocene palladium dichloride (246 mg, 0.378 mmol), (2-chloro-5-methoxypyridin-4-yl)boronic acid (708 mg, 3.78 mmol), and K₂CO₃ (1.57 g, 11.3 mmol) and the mixture was stirred at 80° C. for 4 h under N₂. The mixture was diluted with water (100 mL), extracted with DCM (3×50 mL), and the combined organic layers were washed with brine (100 mL), dried over Na₂SO₄, filtered, and concentrated under reduced pressure. The residue was purified by silica gel chromatography (2-10% EtOAc in pet. ether) to afford ethyl 2′,6-dichloro-5′-methoxy-[4,4′-bipyridine]-3-carboxylate (384 mg, 31%) as a yellow solid. LCMS (ESI, m/z): 327 [M+H]⁺, ¹H NMR (400 MHz, DMSO-d₆) δ 8.84 (s, 1H), 8.25 (s, 1H), 7.73 (s, 1H), 7.58 (s, 1H), 4.14 (q, J=7.2 Hz, 2H), 3.80 (s, 3H), 1.07 (t, J=7.2 Hz, 3H).

Step 2: To a solution of ethyl 2′,6-dichloro-5′-methoxy-[4,4′-bipyridine]-3-carboxylate (235 mg, 0.718 mmol) in DMF (4 mL) was added Cs₂CO₃ (702 mg, 2.15 mmol) and 3-amino-5-chlorophenol (124 mg, 0.862 mmol) and the mixture was stirred at room temperature for 16 h. The mixture was diluted with water (40 mL), extracted with EtOAc (3×20 mL), and the combined organic layers were washed with brine (40 mL), dried over Na₂SO₄, filtered, and concentrated under reduced pressure. The residue was purified by prep-TLC (pet. ether/EtOAc 3/1, v/v) to afford ethyl 6-(3-amino-5-chlorophenoxy)-2′-chloro-5′-methoxy-[4,4′-bipyridine]-3-carboxylate (300 mg, 77% yield) as a yellow solid. LCMS (ESI, m/z): 434 [M+H]⁺. ¹HNMR (400 MHz, DMSO-d₆) δ 8.64 (s, 1H), 8.23 (s, 1H), 7.52 (s, 1H), 7.10 (s, 1H), 6.48 (s, 1H), 6.35 (t, J=2.0 Hz, 1H), 6.29 (t, J=2.0 Hz, 1H), 5.63 (s, 2H), 4.09 (q, J=7.0 Hz, 2H), 3.80 (s, 3H), 1.04 (t, J=7.0 Hz, 3H).

Step 3: To a solution of ethyl 6-(3-amino-5-chlorophenoxy)-2′-chloro-5′-methoxy-[4,4′-bipyridine]-3-carboxylate (282 mg, 80% purity, 0.520 mmol) in MeOH (8 mL) and water (2 mL) was added NaOH (62.3 mg, 1.56 mmol) and the mixture was stirred at 50° C. for 16 h. The mixture was adjusted to pH 3 with a 2 M aqueous HCl solution, diluted with water (15 mL), and extracted with EtOAc (3×10 mL). The combined organic layers were washed with brine (20 mL), dried over Na₂SO₄, filtered, and concentrated under reduced pressure to give 6-(3-amino-5-chlorophenoxy)-2′-chloro-5′-methoxy-[4,4′-bipyridine]-3-carboxylic acid (120 mg, 57% yield) as a brown solid. LCMS (ESI, m/z): 406 [M+H]⁺.

Step 4: To a solution of 6-(3-amino-5-chlorophenoxy)-2′-chloro-5′-methoxy-[4,4′-bipyridine]-3-carboxylic acid (90 mg, 0.22 mmol) in MeCN (3 mL) was added TCFH (93 mg, 0.33 mmol), 6-cyclopropylimidazo[2,1-b][1,3,4]thiadiazol-2-amine (60 mg, 0.33 mmol), and 1-methylimidazole (73 mg, 0.89 mmol) and the mixture was stirred at 80° C. in a sealed tube for 16 h. The mixture was diluted with water (20 mL), extracted with EtOAc (3×10 mL), and the combined organic layers were washed with brine (20 mL), dried over Na₂SO₄, filtered, and concentrated under reduced pressure. The residue was purified by prep-TLC (DCM/MeOH/ACN═40/2/1, v/v) to afford 6-(3-amino-5-chlorophenoxy)-2′-chloro-N-(6-cyclopropylimidazo[2,1-b][1,3,4]thiadiazol-2-yl)-5′-methoxy-[4,4′-bipyridine]-3-carboxamide (2.7 mg, 2%) as a yellow solid. LCMS (ESI, m/z): 568 [M+H]⁺. ¹H NMR (400 MHz, MeOD) δ 8.54 (s, 1H), 8.08 (s, 1H), 7.48 (d, J=3.4 Hz, 2H), 7.02 (s, 1H), 6.58 (t, J=1.8 Hz, 1H), 6.42 (dt, J=8.0, 2.0 Hz, 2H), 3.74 (s, 3H), 2.06-2.00 (m, 1H), 0.88-0.86 (m, 2H), 0.73-0.70 (m, 2H).

### Example 316A & 316B: Synthesis of tert-butyl (3-((2′-chloro-5-((6-cyclopropylimidazo[2,1-b][1,3,4]thiadiazol-2-yl)carbamoyl)-5′-methoxy-[4,4′-bipyridin]-2-yl)oxy)phenyl)carbamate (316A) & 6-(3-Aminophenoxy)-2′-chloro-N-(6-cyclopropylimidazo[2,1-b][1,3,4]thiadiazol-2-yl)-5′-methoxy-[4,4′-bipyridine]-3-carboxamide (316B)

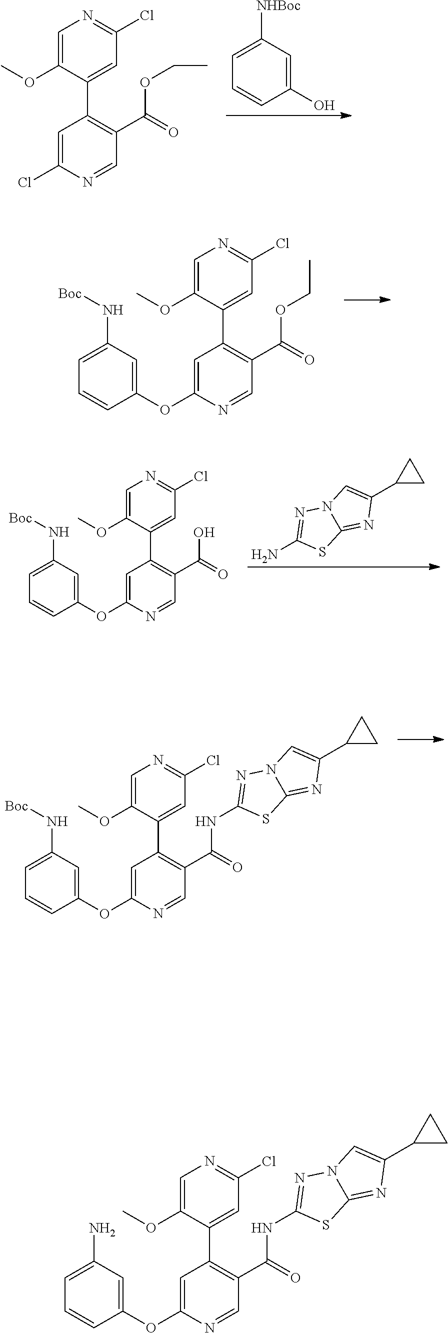

Step 1: A mixture of ethyl 2′,6-dichloro-5′-methoxy-[4,4′-bipyridine]-3-carboxylate (360 mg, 1.10 mmol), Cs₂CO₃ (717 mg, 2.20 mmol), and tert-butyl (3-hydroxyphenyl)carbamate (230 mg, 1.10 mmol) in DMF (6 mL) was stirred at 25° C. for 2 h. The mixture was poured into water (80 mL), extracted with EtOAc (3×30 mL), and the combined organic layers were washed with brine (3×50 mL), dried over Na₂SO₄, filtered, and concentrated under reduced pressure. The residue was purified by prep-TLC (pet. ether/EtOAc=2/1, v/v) to give ethyl 6-(3-((tert-butoxycarbonyl)amino)phenoxy)-2′-chloro-5′-methoxy-[4,4′-bipyridine]-3-carboxylate (460 mg, 83% yield) as an off-white solid. LCMS (ESI, m/z): 500 [M+H]⁺, ¹HNMR (400 MHz, DMSO-d₆) δ 9.51 (s, 1H), 8.61 (s, 1H), 8.23 (s, 1H), 7.54 (s, 1H), 7.38 (s, 1H), 7.31 (t, J=7.8 Hz, 1H), 7.27 (d, J=8.4 Hz, 1H), 7.11 (s, 1H), 6.82-6.77 (m, 1H), 4.09 (q, J=7.0 Hz, 2H), 3.80 (s, 3H), 1.46 (s, 9H), 1.03 (t, J=7.0 Hz, 3H).

Step 2: To a solution of ethyl 6-(3-((tert-butoxycarbonyl)amino)phenoxy)-2′-chloro-5′-methoxy-[4,4′-bipyridine]-3-carboxylate (380 mg, 0.760 mmol) in THF (19 mL) and water (5 mL) was added NaOH (91.2 mg, 2.28 mmol) and the mixture was stirred at 50° C. for 16 h. The mixture was poured into water (30 mL), extracted with EtOAc (3×30 mL), and the combined organic layers were washed with brine (3×30 mL). The aqueous layer was adjusted to pH 6-7, extracted with EtOAc (3×60 mL), and the combined organic layers were dried over Na₂SO₄, filtered and concentrated under reduced pressure to give 6-(3-((tert-butoxycarbonyl)amino)phenoxy)-2′-chloro-5′-methoxy-[4,4′-bipyridine]-3-carboxylic acid (329 mg) as a yellow solid, which was used directly in the next step. LCMS (ESI, m/z): 472 [M+H]⁺, ¹H NMR (400 MHz, DMSO-d₆) δ 9.50 (s, 1H), 8.61 (s, 1H), 8.21 (s, 1H), 7.49 (s, 1H), 7.37 (s, 1H), 7.33-7.25 (m, 2H), 7.05 (s, 1H), 6.79 (d, J=7.4 Hz, 1H), 3.80 (s, 3H), 1.46 (s, 9H).

Step 3: A mixture of 6-(3-((tert-butoxycarbonyl)amino)phenoxy)-2′-chloro-5′-methoxy-[4,4′-bipyridine]-3-carboxylic acid (100 mg, 0.212 mmol), TCFH (89.2 mg, 0.318 mmol), 6-cyclopropylimidazo[2,1-b][1,3,4]thiadiazol-2-amine (57.3 mg, 0.318 mmol), and 1-methylimidazole (69.6 mg, 0.848 mmol) in MeCN (4 mL) was stirred at 80° C. in a sealed tube for 16 h. The mixture was poured into water (40 mL), extracted with EtOAc (3×30 mL), and the combined organic layers were washed with brine (3×40 mL), dried over Na₂SO₄, filtered, and concentrated under reduced pressure. The residue was purified by prep-TLC (DCM/MeOH=20/1, v/v) to give tert-butyl (3-((2′-chloro-5-((6-cyclopropylimidazo[2,1-b][1,3,4]thiadiazol-2-yl)carbamoyl)-5′-methoxy-[4,4′-bipyridin]-2-yl)oxy)phenyl)carbamate (34 mg, 25% yield) as a yellow solid. LCMS (ESI, m/z): 634 [M+H]⁺, ¹H NMR (400 MHz, DMSO-d₆) δ 12.8 (s, 1H), 9.39 (s, 1H), 8.56 (s, 1H), 8.19 (s, 1H), 7.69 (s, 1H), 7.57 (s, 1H), 7.42 (s, 1H), 7.35-7.26 (m, 2H), 7.18 (s, 1H), 6.81 (d, J=7.6 Hz, 1H), 3.69 (s, 3H), 1.94-1.85 (m, 1H), 1.48 (s, 9H), 0.87-0.78 (m, 2H), 0.75-0.68 (m, 2H).

Step 4: To a solution of tert-butyl (3-((2′-chloro-5-((6-cyclopropylimidazo[2,1-b][1,3,4]thiadiazol-2-yl)carbamoyl)-5′-methoxy-[4,4′-bipyridin]-2-yl)oxy)phenyl)carbamate (30 mg, 0.047 mmol) in DCM (6 mL) was added TFA (1.2 mL) and the mixture was stirred at 25° C. for 1 h. The mixture was partitioned between DCM (10 mL) and water (10 mL) and the aqueous phase adjusted to pH 6-7 with a saturated aqueous NaHCO₃ solution. The layers were separated and the aqueous phase extracted with DCM (3×20 mL). The combined organic layers were dried over Na₂SO₄, filtered, and concentrated under reduced pressure. The residue was purified by prep-TLC (DCM/MeOH=20/1, v/v) to give 6-(3-aminophenoxy)-2′-chloro-N-(6-cyclopropylimidazo[2,1-b][1,3,4]thiadiazol-2-yl)-5′-methoxy-[4,4′-bipyridine]-3-carboxamide (21 mg, 84% yield) as a white solid. LCMS (ESI, m/z): 534 [M+H]⁺, ¹H NMR (400 MHz, DMSO-d₆) δ 8.56 (s, 1H), 8.19 (s, 1H), 7.77 (s, 1H), 7.61 (s, 1H), 7.14 (s, 1H), 7.12-7.05 (m, 1H), 6.51-6.46 (m, 1H), 6.40-6.36 (m, 1H), 6.36-6.31 (m, 1H), 3.67 (s, 3H), 1.92-1.87 (m, 1H), 0.85-0.81 (m, 2H), 0.74-0.69 (m, 2H). 3 exchangeable Hs not observed.

### Example 317: Synthesis of (cis or trans)-4-(5-cyano-2-methoxyphenyl)-N-(6-(4-cyanocyclohexyl)thiazolo[4,5-b]pyrazin-2-yl)-6-methylnicotinamide with unknown stereochemistry

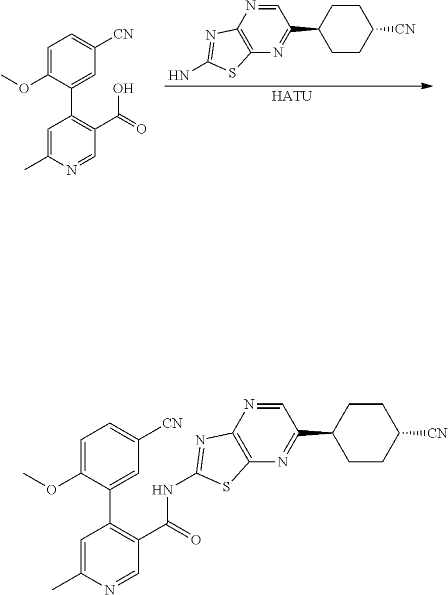

To a solution of 4-(5-cyano-2-methoxyphenyl)-6-methylnicotinic acid (81 mg, 0.30 mmol) and HATU (0.11 g, 0.30 mmol) in DMF (2 mL) was added DIPEA (98 mg, 0.75 mmol) and the mixture was stirred at room temperature for 2 h. (Cis or Trans)-4-(2-aminothiazolo[4,5-b]pyrazin-6-yl)cyclohexane-1-carbonitrile (39 mg, 0.15 mmol) was added and the mixture was stirred at 80° C. for 20 h. The mixture was diluted with water (20 mL), extracted with EtOAc (20 mL×2), and the combined organic layers were washed with water (20 mL), brine (20 mL), dried over Na₂SO₄, filtered, and concentrated under reduced pressure. The residue was purified by prep-HPLC to afford (cis or trans)-4-(5-cyano-2-methoxyphenyl)-N-(6-(4-cyanocyclohexyl)thiazolo[4,5-b]pyrazin-2-yl)-6-methylnicotinamide (18 mg, 23%) as an orange solid. LCMS (ESI, m/z): 510 [M+H]; ¹H NMR (400 MHz, DMSO-d₆) δ 13.3 (s, 1H), 8.80 (s, 1H), 8.54 (s, 1H), 7.95-7.88 (m, 2H), 7.44 (s, 1H), 7.16 (d, J=9.2 Hz, 1H), 3.58 (s, 3H), 3.00-2.89 (m, 1H), 2.86-2.74 (m, 1H), 2.60 (s, 3H), 2.20-2.11 (m, 2H), 1.98-1.88 (m, 2H), 1.75-1.59 (m, 4H).

TABLE AS
The following examples were prepared using a similar procedure to that described for
Example 317

| Ex. | | | |
|---|---|---|---|
| No. | Structure | Compound Name | Characterization Data |
| 318^{%} | 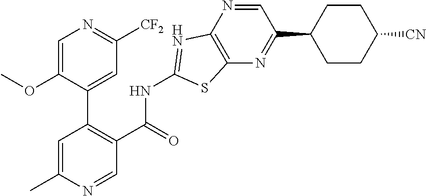 | (cis or trans)-N-(6-(4- Cyanocyclohexyl) thiazolo [4,5-b]pyrazin-2- yl)-2′- (difluoromethyl)-5′- methoxy-6-methyl- [4,4′-bipyridine]-3- carboxamide | LCMS (m/z): 536 [M + H]⁺. ¹H NMR (400 MHz, DMSO-d₆) δ 13.4 (s, 1H), 8.89 (s, 1H), 8.52 (s, 1H), 8.45 (s, 1H), 7.73 (s, 1H), 7.45 (s, 1H), 6.99 (t, J = 55.0 Hz, 1H), 3.69 (s, 3H), 2.99-2.87 (m, 1H), 2.86- 2.75 (m, 1H), 2.61 (s, 3H), 2.21- 2.09 (m, 2H), 1.98-1.88 (m, 2H), 1.75-1.59 (m, 4H) |
| 319^{%} | 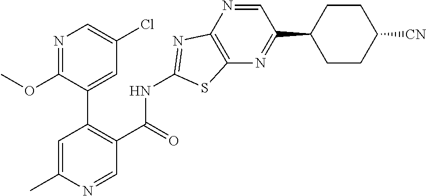 [SMILES: COc1ncc(Cl)cc1-c1cc(C)ncc1C(=O)Nc1nc2ncc([C@H]3CC[C@H](C#N)CC3)nc2s1] | (cis or trans)-5- Chloro-N-(6-(4- cyanocyclohexyl) thiazolo [4,5-b]pyrazin-2- yl)-2-methoxy-6′- methyl-[3,4′- bipyridine]-3′- carboxamide | LCMS (m/z): 520 [M + H]⁺. ¹H NMR (400 MHz, DMSO-d₆) δ 13.4 (s, 1H), 8.84 (s, 1H), 8.56 (s, 1H), 8.28 (d, J = 2.6 Hz, 1H), 8.04 (d, J = 2.6 Hz, 1H), 7.50 (s, 1H), 3.58 (s, 3H), 2.99-2.91 (m, 1H), 2.84- 2.75 (m, 1H), 2.60 (s, 3H), 2.20- 2.11 (m, 2H), 1.97-1.90 (m, 2H), 1.74-1.60 (m, 4H) |
| 320^{%} | 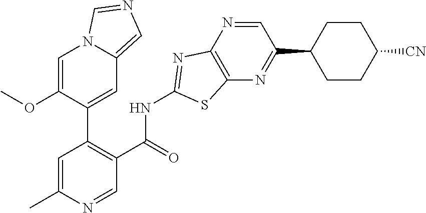 [SMILES: COc1cn2cncc2cc1-c1cc(C)ncc1C(=O)Nc1nc2ncc([C@H]3CC[C@H](C#N)CC3)nc2s1] | (cis or trans)-N-(6-(4- Cyanocyclohexyl) thiazolo[4,5-b]pyrazin- 2-yl)-4-(6- methoxyimidazo[1,5- a]pyridin-7-yl)-6- methylnicotinamide | LCMS (m/z): 525 [M + H]⁺. ¹H NMR (400 MHz, DMSO-d₆) δ 13.4 (s, 1H), 9.00 (br s, 1H), 8.87 (s, 1H), 8.57 (s, 1H), 8.15 (s, 1H), 7.98- 7.88 (m, 2H), 7.50 (s, 1H), 3.52 (s, 3H), 2.96-2.92 (m, 1H), 2.81- 2.76 (m, 1H), 2.63 (s, 3H), 2.18- 2.11 (m, 2H), 1.97-1.90 (m, 2H), 1.74-1.59 (m, 4H) |
| 321ⁿ | 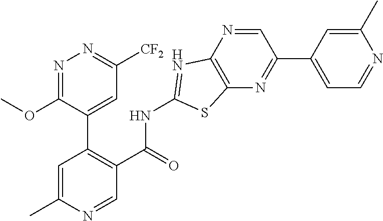 | 4-(6- (difluoromethyl)-3- methoxypyridazin-4- yl)-6-methyl-N-(6-(2- methylpyridin-4- yl)thiazolo[4,5- b]pyrazin-2- yl)nicotinamide | LCMS (m/z): 521 [M + H]⁺. ¹H NMR (400 MHz, DMSO-d₆) δ 13.76 (s, 1H), 9.35 (s, 1H), 9.02 (s, 1H), 8.60 (d, J = 5.3 Hz, 1H), 8.14 (s, 1H), 8.02 (s, 1H), 7.94 (dd, J = 5.3, 1.8 Hz, 1H), 7.61 (s, 1H), 7.23 (d, J = 54.2 Hz, 1H), 3.87 (s, 3H), 2.65 (s, 3H), 2.58 (s, 3H) |

^{%}Unknown stereochemistry
ⁿReaction for the synthesis of Example 321 was run at room temperature instead of 80° C.

### Example 322: Synthesis of (cis or trans)-N-(6-(4-Cyanocyclohexyl)thiazolo[4,5-b]pyrazin-2-yl)-4-(2-fluoro-6-methoxyphenyl)-6-methylnicotinamide with unknown stereochemistry

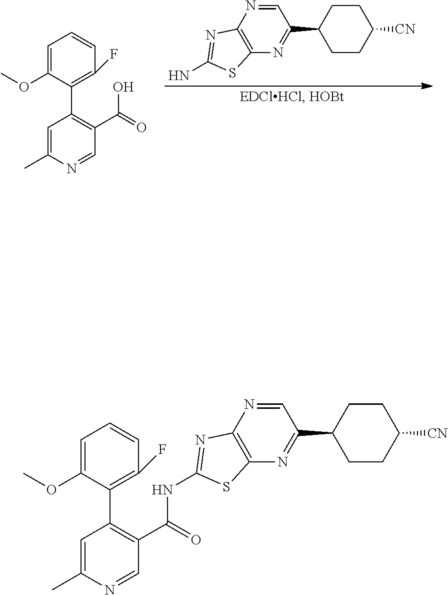 [SMILES: COc1cccc(F)c1-c1cc(C)ncc1C(=O)O ; N#C[C@H]1CC[C@H](c2cnc3nc(N)sc3n2)CC1 ; "EDCl·HCl, HOBt" ; COc1cccc(F)c1-c1cc(C)ncc1C(=O)Nc1nc2ncc([C@H]3CC[C@H](C#N)CC3)nc2s1]

To a solution of 4-(2-fluoro-6-methoxyphenyl)-6-methylnicotinic acid (60 mg, 0.23 mmol), (cis or trans)-4-(2-aminothiazolo[4,5-b]pyrazin-6-yl)cyclohexane-1-carbonitrile (30 mg, 0.11 mmol) and DIPEA (0.12 g, 0.92 mmol) in acetonitrile (10 mL) and NMP (2 mL) was added EDCI·HCl (66 mg, 0.34 mmol) and HOBt (46 mg, 0.30 mmol) and the mixture was stirred at room temperature overnight. The mixture was diluted with water (50 mL), extracted with EtOAc (40 mL×2), and the combined organic layers were washed with water (40 mL), brine (40 mL), dried over Na₂SO₄, filtered, and concentrated under reduced pressure. The residue was purified by prep-TLC (DCM/MeOH=30/1, v/v) to afford (cis or trans)-N-(6-(4-cyanocyclohexyl)thiazolo[4,5-b]pyrazin-2-yl)-4-(2-fluoro-6-methoxyphenyl)-6-methylnicotinamide (33 mg, 29% yield) as a white solid. LCMS (ESI, m/z): 503 [M+H]f; ¹H NMR (400 MHz, DMSO-d₆) δ 13.3 (s, 1H), 8.87 (s, 1H), 8.55 (s, 1H), 7.47-7.34 (m, 2H), 6.95 (t, J=9.0 Hz, 1H), 6.88 (d, J=8.4 Hz, 1H), 3.57 (s, 3H), 2.98-2.89 (m, 1H), 2.84-2.76 (m, 1H), 2.59 (s, 3H), 2.18-2.11 (m, 2H), 1.97-1.89 (m, 2H), 1.73-1.61 (m, 4H).

TABLE AT
The following examples were prepared using a similar procedure to that described for
Example 322

| Ex. | | | |
|---|---|---|---|
| No. | Structure | Compound Name | Characterization Data |
| 323 | 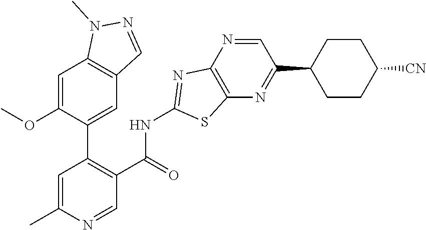 [SMILES: COc1cc2c(cnn2C)cc1-c1cc(C)ncc1C(=O)Nc1nc2ncc([C@H]3CC[C@H](C#N)CC3)nc2s1] | (cis or trans)-N-(6- (4- Cyanocyclohexyl) thiazolo[4,5- b]pyrazin-2-yl)-4- (6-methoxy-1- methyl-1H-indazol- 5-yl)-6- methylnicotinamide | LCMS (m/z): 539 [M + H]⁺. ¹H NMR (400 MHz, DMSO-d₆) δ 13.2 (s, 1H), 8.76 (s, 1H), 8.56 (s, 1H), 8.04 (s, 1H), 7.80 (s, 1H), 7.42 (s, 1H), 7.10 (s, 1H), 4.02 (s, 3H), 3.60 (s, 3H), 2.98-2.89 (m, 1H), 2.84-2.75 (m, 1H), 2.62 (s, 3H), 2.18-2.11 (m, 2H), 1.96-1.88 (m, 2H), 1.75- 1.62 (m, 4H) |
| 324^{o} | 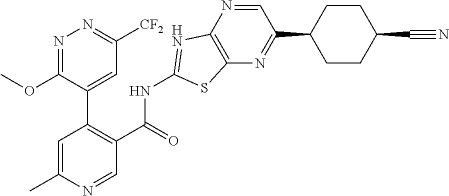 | (cis or trans)-N-(6- (4- cyanocyclohexyl) thiazolo[4,5- b]pyrazin-2-yl)-4- (6- (difluoromethyl)-3- methoxypyridazin- 4-yl)-6- methylnicotinamide | LCMS (m/z): 537 [M + H]⁺. ¹H NMR (400 MHz, DMSO-d₆) δ 13.51 (s, 1H), 9.02 (s, 1H), 8.53 (s, 1H), 8.09 (s, 1H), 7.56 (s, 1H), 7.28 (t, J = 54.2 Hz, 1H), 3.85 (s, 3H), 3.28- 3.21 (m, 1H), 3.00-2.90 (m, 1H), 2.63 (s, 3H), 2.05-1.97 (m, 2H), 1.97-1.90 (m, 2H), 1.89-1.64 (m, 4H) |
| 325^{o} | 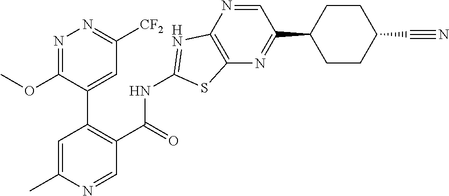 | (trans or cis)-N-(6- (4- cyanocyclohexyl) thiazolo[4,5- b]pyrazin-2-yl)-4- (6- (difluoromethyl)-3- methoxypyridazin- 4-yl)-6- methylnicotinamide | LCMS (m/z): 537 [M + H]⁺. ¹H NMR (400 MHz, DMSO-d₆) δ 13.52 (s, 1H), 9.02 (d, J = 10.3 Hz, 1H), 8.65 (d, J = 112.3 Hz, 1H), 8.08 (d, J = 7.5 Hz, 1H), 7.54 (d, J = 7.8 Hz, 1H), 7.27 (t, J = 54.2 Hz, 1H), 3.85 (d, J = 3.8 Hz, 3H), 3.17 (d, J = 6.7 Hz, 1H), 2.92 (s, 1H), 2.80 (s, 1H), 2.63 (s, 4H), 2.14 (d, J = 8.8 Hz, 1H), 1.97 (dd, J = 28.6, 8.2 Hz, 2H), 1.78-1.57 (m, 3H) |

^{o}Chiral separation conditions for Example 324 and 325: Prep-HPLC Xselect CSH C18 OBD Column 30 × 150 mm, 5 μm; 49-65% MeOH in water with 0.1% formic acid modifier; 60 mL/min flow rate; wave length: 254 nm/220 nm

### Example 326: Synthesis of (S)-2′-chloro-N-(6-(3-(hydroxymethyl)pyrrolidin-1-yl)thiazolo[4,5-b]pyrazin-2-yl)-5′-methoxy-6-methyl-[4,4′-bipyridine]-3-carboxamide

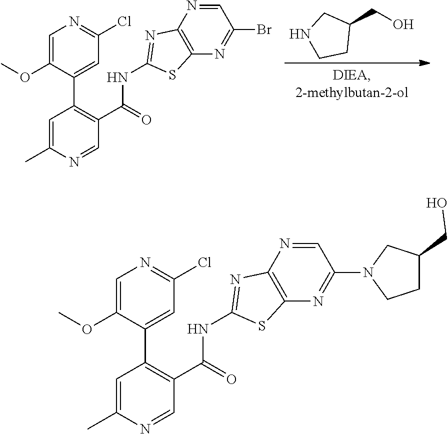 [SMILES: COc1cnc(Cl)cc1-c1cc(C)ncc1C(=O)Nc1nc2ncc(Br)nc2s1 ; OC[C@H]1CCNC1 ; COc1cnc(Cl)cc1-c1cc(C)ncc1C(=O)Nc1nc2ncc(N3CC[C@H](CO)C3)nc2s1]

A solution of N-(6-bromothiazolo[4,5-b]pyrazin-2-yl)-2′-chloro-5′-methoxy-6-methyl-[4,4′-bipyridine]-3-carboxamide (100 mg, 0.2 mmol) in 2-methylbutan-2-ol (5 mL) was treated with (S)-pyrrolidin-3-ylmethanol (41 mg, 0.4 mmol) and DIEA (79 mg, 0.6 mmol) at 25° C. under a nitrogen atmosphere. The reaction mixture was stirred at 140° C. for 12 h under a nitrogen atmosphere. The resulting mixture was cooled to 25° C. and concentrated under reduced pressure. The residue was purified directly by reverse-phase flash chromatography (10-50% MeCN in water with 0.1% formic acid modifier) to afford the crude product. The crude product was purified by prep-HPLC with the following conditions (Column: Sunfire prep C18 column, 30*150 mm, 5 μm; Mobile Phase A: water (0.1% FA), Mobile Phase B: ACN; Flow rate: 60 mL/min; Gradient: 15% B to 35% B in 10 min, 95% B; Wave Length: 254/220 nm; RT1 (min): 12.72; Number of Runs: 1) to afford (S)-2′-chloro-N-(6-(3-(hydroxymethyl)pyrrolidin-1-yl)thiazolo[4,5-b]pyrazin-2-yl)-5′-methoxy-6-methyl-[4,4′-bipyridine]-3-carboxamide (22.5 mg, 21.6% yield) as a yellow solid. LCMS (ESI, m/z): 512 [M+H]⁺. ¹H NMR (400 MHz, DMSO-d₆) δ 12.91 (s, 1H), 8.84 (s, 1H), 8.17 (s, 1H), 7.95 (s, 1H), 7.57 (s, 1H), 7.45 (s, 1H), 4.74 (t, J=5.2 Hz, 1H), 3.63 (s, 3H), 3.61-3.52 (m, 2H), 3.51-3.38 (m, 3H), 3.27-3.24 (m, 1H), 2.60 (s, 3H), 2.48-2.39 (m, 1H), 2.10-1.02 (m, 1H), 1.83-1.74 (m, 1H).

TABLE AU
The following examples were prepared using a similar procedure to that describedf or
Example 326

| Ex. | | | |
|---|---|---|---|
| No. | Structure | Compound Name | Characterization Data |
| 327 | 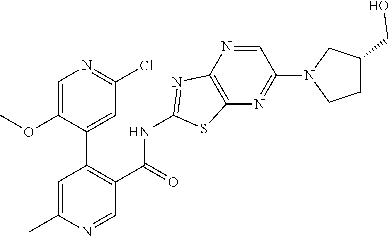 [SMILES: COc1cnc(Cl)cc1-c1cc(C)ncc1C(=O)Nc1nc2ncc(N3CC[C@@H](CO)C3)nc2s1] | (R)-2′-chloro-N-(6-(3- (hydroxymethyl)pyrrolidin- 1-yl)thiazolo[4,5- b]pyrazin-2-yl)-5′- methoxy-6-methyl- [4,4′-bipyridine]-3- carboxamide | LCMS (m/z): 512 [M + H]⁺. ¹H NMR (300 MHz, Methanol-d4) δ 8.83 (s, 1H), 8.09 (s, 1H), 7.89 (s, 1H), 7.52 (s, 1H), 7.45 (s, 1H), 3.74 (s, 3H), 3.73-3.52 (m, 5H), 3.42-3.35 (m, 1H), 2.70 (s, 3H), 2.67-2.54 (m, 1H), 2.26-2.15 (m, 1H), 1.98-1.83 (m, 1H) |
| 328 | 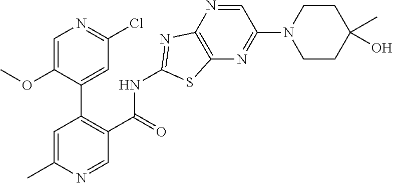 | 2′-chloro-N-(6-(4- hydroxy-4- methylpiperidin-1- yl)thiazolo[4,5- b]pyrazin-2-yl)-5′- methoxy-6-methyl- [4,4′-bipyridine]-3- carboxamide | LCMS (m/z): 526 [M + H]⁺. ¹H NMR (400 MHz, Methanol-d₄) δ 8.86 (s, 1H), 8.22 (s, 1H), 8.09 (s, 1H), 7.53 (s, 2H), 4.06-3.96 (m, 2H), 3.72 (s, 3H), 3.57-3.45 (m, 2H), 2.71 (s, 3H), 1.77-1.63 (m, 4H), 1.26 (s, 3H) |
| 329 | 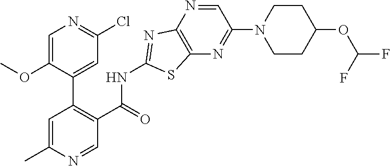 [SMILES: COc1cnc(Cl)cc1-c1cc(C)ncc1C(=O)Nc1nc2ncc(N3CCC(OC(F)F)CC3)nc2s1] | 2′-chloro-N-{6-[4- (difluoromethoxy) piperidin- 1-yl]- [1,3]thiazolo[4,5- b]pyrazin-2-yl}-5′- methoxy-6-methyl- [4,4′-bipyridine]-3- carboxamide | LCMS (m/z): 562 [M + H]⁺. ¹H NMR (400 MHz, DMSO-d₆) δ 12.98 (s, 1H), 8.84 (s, 1H), 8.37 (s, 1H), 8.16 (s, 1H), 7.57 (s, 1H), 7.45 (s, 1H), 6.78 (s, 1H), 4.45- 4.36 (m, 1H), 4.05-3.94 (m, 2H), 3.62 (s, 3H), 3.41-3.32 (m, 2H), 2.60 (s, 3H), 2.03-1.93 (m, 2H), 1.72-1.59 (m, 2H) |
| 330 | 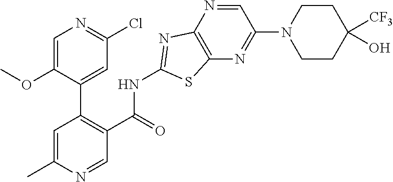 [SMILES: COc1cnc(Cl)cc1-c1cc(C)ncc1C(=O)Nc1nc2ncc(N3CCC(O)(C(F)(F)F)CC3)nc2s1] | 2′-chloro-N-(6-(4- hydroxy-4- (trifluoromethyl) piperidin- 1-yl)thiazolo[4,5- b]pyrazin-2-yl)-5′- methoxy-6-methyl- [44,4′-bipyridine]-3- carboxamide | LCMS (m/z): 580 [M + H]⁺. ¹H NMR (400 MHz, DMSO-d₆) δ 12.99 (s, 1H), 8.84 (s, 1H), 8.40 (s, 1H), 8.17 (s, 1H), 7.58 (s, 1H), 7.46 (s, 1H), 6.12 (s, 1H), 4.32 (d, J = 13.3 Hz, 2H), 3.63 (s, 3H), 3.18-3.15 (m, 2H), 2.60 (s, 3H), 1.82-1.66 (m, 4H) |
| 331 | 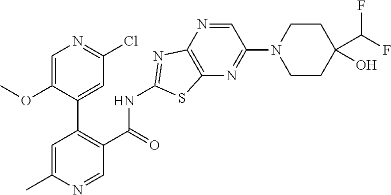 [SMILES: COc1cnc(Cl)cc1-c1cc(C)ncc1C(=O)Nc1nc2ncc(N3CCC(O)(C(F)F)CC3)nc2s1] | 2′-chloro-N-(6-(4- (difluoromethyl)-4- hydroxypiperidin-1- yl)thiazolo[4,5- b]pyrazin-2-yl)-5′- methoxy-6-methyl- [4,4′-bipyridine]-3- carboxamide | LCMS (m/z): 562 [M + H]⁺. ¹H NMR (400 MHz, DMSO-d₆) δ 12.97 (s, 1H), 8.83 (s, 1H), 8.38 (s, 1H), 8.17 (s, 1H), 7.58 (s, 1H), 7.46 (s, 1H), 5.74 (t, J = 56.0 Hz, 1H), 5.39 (s, 1H), 4.25 (d, J = 13.2 Hz, 2H), 3.62 (s, 3H), 3.26- 3.15 (m, 2H), 2.60 (s, 3H), 1.64- 1.55 (m, 4H) |
| 332 | 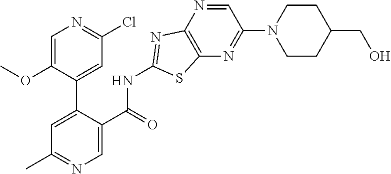 [SMILES: COc1cnc(Cl)cc1-c1cc(C)ncc1C(=O)Nc1nc2ncc(N3CCC(CO)CC3)nc2s1] | 2′-chloro-N-(6-(4- (hydroxymethyl)piperidin- 1-yl)thiazolo[4,5- b]pyrazin-2-yl)-5′- methoxy-6-methyl- [4,4′-bipyridine]-3- carboxamide | LCMS (m/z): 526 [M + H]⁺. ¹H NMR (300 MHz, Methanol-d₄) δ 8.81 (s, 1H), 8.20 (s, 1H), 8.07 (s, 1H), 7.52 (s, 1H), 7.44 (s, 1H), 4.46 (s, 2H), 3.72 (s, 3H), 3.44 (d, J = 6.2 Hz, 2H), 3.00 (d, J = 12.4 Hz, 2H), 2.68 (s, 3H), 1.84 (s, 3H), 1.36 (s, 2H) |
| 333 | 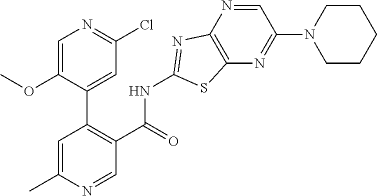 [SMILES: COc1cnc(Cl)cc1-c1cc(C)ncc1C(=O)Nc1nc2ncc(N3CCCCC3)nc2s1] | 2′-chloro-5′-methoxy-6- methyl-N-(6-(piperidin- 1-yl)thiazolo[4,5- b]pyrazin-2-yl)-[4,4′- bipyridine]-3- carboxamide | LCMS (m/z): 496 [M + H]⁺. ¹H NMR (400 MHz, DMSO-d₆) δ 12.94 (s, 1H), 8.84 (s, 1H), 8.31 (s, 1H), 8.16 (s, 1H), 7.56 (s, 1H), 7.44 (s, 1H), 3.63 (s, 3H), 3.60 (s, 4H), 2.60 (s, 3H), 1.61 (s, 6H) |
| 334 | 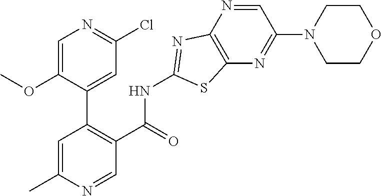 [SMILES: COc1cnc(Cl)cc1-c1cc(C)ncc1C(=O)Nc1nc2ncc(N3CCOCC3)nc2s1] | 2′-chloro-5′-methoxy-6- methyl-N-(6- morpholinothiazolo[4,5- b]pyrazin-2-yl)-[4,4′- bipyridine]-3- carboxamide | LCMS (m/z): 498 [M + H]⁺. ¹H NMR (400 MHz, DMSO-d₆) δ 13.01 (s, 1H), 8.84 (s, 1H), 8.34 (s, 1H), 8.17 (s, 1H), 7.57 (s, 1H), 7.46 (s, 1H), 3.74 (t, J = 4.8 Hz, 4H), 3.62 (s, 3H), 3.55 (t, J = 4.8 Hz, 4H), 2.60 (s, 3H) |
| 335 | 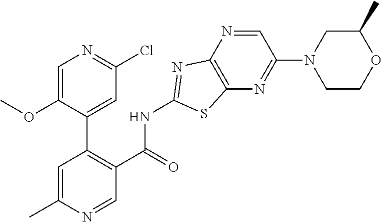 | (R)-2′-chloro-5′- methoxy-6-methyl-N- (6-(2- methylmorpholino) thiazolo[4,5-b]pyrazin-2- yl)-[4,4′-bipyridine]-3- carboxamide | LCMS (m/z): 512 [M + H]⁺. ¹H NMR (400 MHz, DMSO-d₆) δ 13.00 (s, 1H), 8.84 (s, 1H), 8.35 (s, 1H), 8.17 (s, 1H), 7.58 (s, 1H), 7.46 (s, 1H), 4.21-4.18 (m, 1H), 4.12-4.08 (m, 1H), 3.96-3.92 (m, 1H), 3.62 (s, 3H), 3.62-3.55 (m, 2H), 2.99-2.89 (m, 1H), 2.60 (s, 4H), 1.18 (d, J = 6.2 Hz, 3H) |
| 336 | 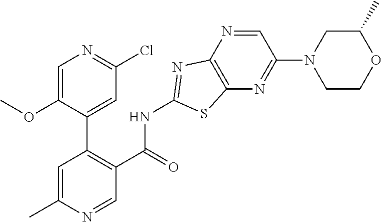 | (S)-2′-chloro-5′- methoxy-6-methyl-N- (6-(2- methylmorpholino) thiazolo[4,5-b]pyrazin-2- yl)-[4,4′-bipyridine]-3- carboxamide | LCMS (m/z): 512 [M + H]⁺. ¹H NMR (400 MHz, DMSO-d₆) δ 13.01 (s, 1H), 8.84 (s, 1H), 8.35 (s, 1H), 8.17 (s, 1H), 7.58 (s, 1H), 7.46 (s, 1H), 4.20-4.08 (m, 2H), 3.95-3.93 (m, 1H), 3.68-3.50 (m, 5H), 2.97-2.90 (m, 1H), 2.69- 2.55 (m, 4H), 1.18 (d, J = 6.2 Hz, 3H) |
| 337 | 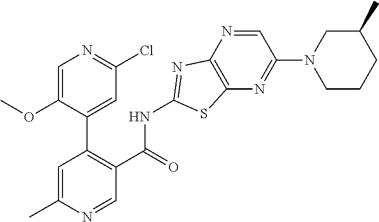 | (S)-2′-chloro-5′- methoxy-6-methyl-N- (6-(3-methylpiperidin- 1-yl)thiazolo[4,5- b]pyrazin-2-yl)-[4,4′- bipyridine]-3- carboxamide | LCMS (m/z): 510 [M + H]⁺. ¹H NMR (400 MHz, DMSO-d₆) δ 12.96 (s, 1H), 8.85 (d, J = 2.0 Hz, 1H), 8.34 (s, 1H), 8.17 (s, 1H), 7.58 (s, 1H), 7.53-7.47 (m, 1H), 4.33-4.17 (m, 2H), 3.63 (s, 3H), 2.89-2.86 (m, 1H), 2.66-2.54 (m, 4H), 1.88-1.68 (m, 2H), 1.68- 1.43 (m, 2H), 1.26-1.09 (m, 1H), 0.93 (d, J = 6.6 Hz, 3H) |
| 338 | 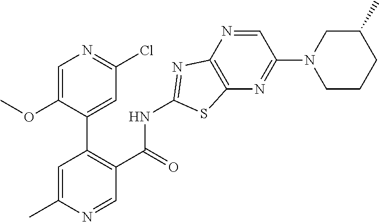 | (R)-2′-chloro-5′- methoxy-6-methyl-N- (6-(3-methylpiperidin- 1-yl)thiazolo[4,5- b]pyrazin-2-yl)-[4,4′- bipyridine]-3- carboxamide | LCMS (m/z): 510 [M + H]⁺. ¹H NMR (300 MHz, DMSO-d₆) δ 12.93 (s, 1H), 8.84 (s, 1H), 8.33 (s, 1H), 8.17 (s, 1H), 7.57 (s, 1H), 7.45 (s, 1H), 4.31-4.17 (m, 2H), 3.63 (s, 3H), 2.93-2.84 (m, 1H), 2.62-2.58 (m, 4H), 1.85-1.40 (m, 4H), 1.23-1.09 (m, 1H), 0.93 (d, J = 6.6 Hz, 3H) |
| 339^{~,p} | 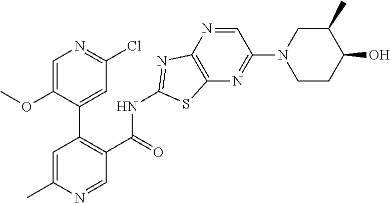 [SMILES: COc1cnc(Cl)cc1-c1cc(C)ncc1C(=O)Nc1nc2ncc(N3CC[C@H](O)[C@H](C)C3)nc2s1] | 2′-chloro-N-(6-((3R or S,4S and R OR 3R and S,4S or R)-4-hydroxy- 3-methylpiperidin-1- yl)thiazolo[4,5- b]pyrazin-2-yl)-5′- methoxy-6-methyl- [4,4′-bipyridine]-3- carboxamide | LCMS (m/z): 526 [M + H]⁺. ¹H NMR (400 MHz, DMSO-d₆) δ 12.93 (s, 1H), 8.83 (s, 1H), 8.34 (s, 1H), 8.17 (s, 1H), 7.57 (s, 1H), 7.46 (s, 1H), 3.82-3.77 (m, 3H), 3.62 (s, 3H), 3.43 (d, J = 8.5 Hz, 2H), 3.23-3.15 (m, 1H), 2.60 (s, 3H), 1.83-1.59 (m, 3H), 0.91 (d, J = 6.9 Hz, 3H) |
| 340^{+,p} | 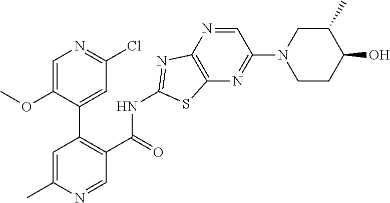 [SMILES: COc1cnc(Cl)cc1-c1cc(C)ncc1C(=O)Nc1nc2ncc(N3CC[C@H](O)[C@@H](C)C3)nc2s1] | 2′-chloro-N-(6-((3S or R,4S and R OR 3S and R,4S or R)-4-hydroxy- 3-methylpiperidin-1- yl)thiazolo[4,5- b]pyrazin-2-yl)-5′- methoxy-6-methyl- [4,4′-bipyridine]-3- carboxamide | LCMS (m/z): 526 [M + H]⁺. ¹H NMR (400 MHz, DMSO-d₆) δ 12.95 (s, 1H), 8.84 (s, 1H), 8.36 (s, 1H), 8.16 (s, 1H), 7.57 (s, 1H), 7.45 (s, 1H), 4.70 (d, J = 5.4 Hz, 1H), 4.26-4.19 (m, 2H), 3.62 (s, 3H), 3.21 (d, J = 9.8, 4.9 Hz, 1H), 3.01-2.98 (m, 1H), 2.69-2.66 (m, 1H), 2.60 (s, 3H), 1.89-1.87 (m, 1H), 1.48-1.35 (m, 2H), 0.98 (d, J = 6.5 Hz, 3H) |
| 341 | 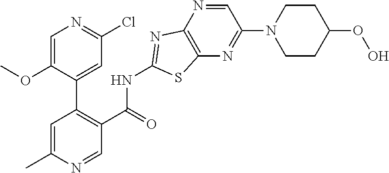 | 2′-chloro-5′-methoxy-6- methyl-N-(6-(4- (trifluoromethoxy) piperidin- 1-yl)thiazolo[4,5- b]pyrazin-2-yl)-[4,4′- bipyridine]-3- carboxamide | LCMS (m/z): 580 [M + H]⁺. ¹H NMR (400 MHz, DMSO-d₆) δ 13.00 (s, 1H), 8.84 (s, 1H), 8.39 (s, 1H), 8.17 (s, 1H), 7.58 (s, 1H), 7.46 (s, 1H), 3.93-3.87 (m, 2H), 3.63 (s, 3H), 3.44-3.31 (m, 2H), 3.17-3.13 (m, 1H), 2.60 (s, 3H), 2.11-1.96 (m, 2H), 1.75 (m, 2H) |
| 342 | 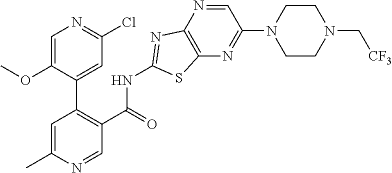 [SMILES: COc1cnc(Cl)cc1-c1cc(C)ncc1C(=O)Nc1nc2ncc(N3CCN(CC(F)(F)F)CC3)nc2s1] | 2′-chloro-5′-methoxy-6- methyl-N-{6-[4-(2,2,2- trifluoroethyl)piperazin- 1-yl]-[1,3]thiazolo[4,5- b]pyrazin-2-yl}-[4,4′- bipyridine]-3- carboxamide | LCMS (m/z): 579 [M + H]⁺. ¹H NMR (400 MHz, DMSO-d₆) δ 13.00 (s, 1H), 8.86 (s, 1H), 8.32 (s, 1H), 8.16 (s, 1H), 7.55 (s, 1H), 7.43 (s, 1H), 3.62 (s, 3H), 3.59 (s, 4H), 3.26-3.22 (m, 2H), 2.75 (s, 4H), 2.60 (s, 3H) |
| 343 | 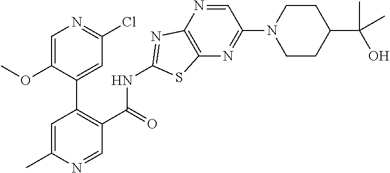 [SMILES: COc1cnc(Cl)cc1-c1cc(C)ncc1C(=O)Nc1nc2ncc(N3CCC(C(C)(C)O)CC3)nc2s1] | 2′-chloro-N-(6-(4-(2- hydroxypropan-2- yl)piperidin-1- yl)thiazolo[4,5- b]pyrazin-2-yl)-5′- methoxy-6-methyl- [4,4′-bipyridine]-3- carboxamide | LCMS (m/z): 554 [M + H]⁺. ¹H NMR (400 MHz, DMSO-d6) δ 12.95 (s, 1H), 8.84 (s, 1H), 8.33 (s, 1H), 8.17 (d, J = 1.6 Hz, 1H), 7.57 (s, 1H), 7.45 (s, 1H), 4.45- 4.41 (m, 2H), 4.15 (d, J = 1.5 Hz, 1H), 3.63 (d, J = 1.6 Hz, 3H), 2.83- 2.79 (m, 2H), 2.60 (d, J = 1.6 Hz, 3H), 1.83-1.79 (m, 2H), 1.48-1.44 (m, 1H), 1.27-1.24 (m, 2H), 1.05 (d, J = 1.6 Hz, 6H) |
| 344 | 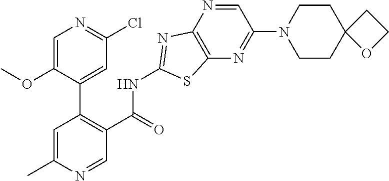 [SMILES: COc1cnc(Cl)cc1-c1cc(C)ncc1C(=O)Nc1nc2ncc(N3CCC4(CCO4)CC3)nc2s1] | 2′-chloro-5′-methoxy-6- methyl-N-(6-{1-oxa-7- azaspiro[3.5]nonan-7- yl}-[1,3]thiazolo[4,5- b]pyrazin-2-yl)-[4,4′- bipyridine]-3- carboxamide | LCMS (m/z): 538 [M + H]⁺. ¹H NMR (400 MHz, Methanol-d₄) δ 8.81 (s, 1H), 8.15 (s, 1H), 8.07 (s, 1H), 7.51 (s, 1H), 7.44 (s, 1H), 5.62 (s, 1H), 4.05 (s, 2H), 3.83 (t, J = 5.7 Hz, 2H), 3.82 (s, 2H),3.72- 3.64 (m, 2H), 2.68 (s, 3H), 2.34- 2.26 (m, 4H) |
| 345 | 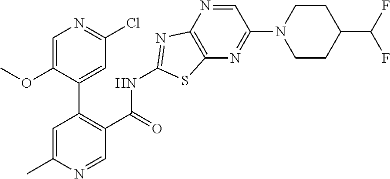 [SMILES: COc1cnc(Cl)cc1-c1cc(C)ncc1C(=O)Nc1nc2ncc(N3CCC(C(F)F)CC3)nc2s1] | 2′-chloro-N-(6-(4- (difluoromethyl)piperidin- 1-yl)thiazolo[4,5- b]pyrazin-2-yl)-5′- methoxy-6-methyl- [4,4′-bipyridine]-3- carboxamide | LCMS (m/z): 546 [M + H]⁺. ¹H NMR (400 MHz, DMSO-d₆) δ 12.98 (s, 1H), 8.83 (s, 1H), 8.37 (s, 1H), 8.17 (s, 1H), 7.58 (s, 1H), 7.46 (s, 1H), 6.09-5.79 (m, 1H), 4.44-4.41 (m, 2H), 3.62 (s, 3H), 3.01-2.90 (m, 2H), 2.60 (s, 3H), 2.19-2.08 (m, 1H), 1.81-1.77 (m, 2H), 1.45-1.33 (m, 2H) |
| 346 | 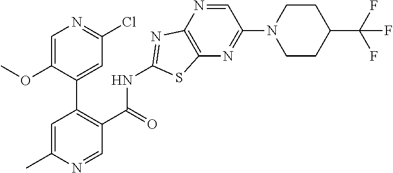 [SMILES: COc1cnc(Cl)cc1-c1cc(C)ncc1C(=O)Nc1nc2ncc(N3CCC(C(F)(F)F)CC3)nc2s1] | 2′-chloro-5′-methoxy-6- methyl-N-(6-(4- (trifluoromethyl)piperidin- 1-yl)thiazolo[4,5- b]pyrazin-2-yl)-[4,4′- bipyridine]-3- carboxamide | LCMS (m/z): 564 [M + H]⁺. ¹H NMR (400 MHz, DMSO-d6) δ 13.00 (s, 1H), 8.84 (s, 1H), 8.38 (s, 1H), 8.17 (s, 1H), 7.58 (s, 1H), 7.46 (s, 1H), 4.48-4.44 (m, 2H), 3.62 (s, 3H), 2.99-2.94 (m, 2H), 2.67 (s, 1H), 2.60 (s, 3H), 1.94- 1.90 (m, 2H), 1.51-1.47 (m, 2H) |
| 347 | 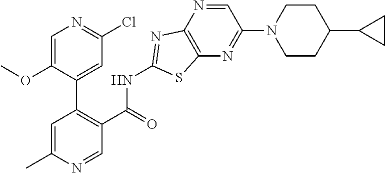 [SMILES: COc1cnc(Cl)cc1-c1cc(C)ncc1C(=O)Nc1nc2ncc(N3CCC(C4CC4)CC3)nc2s1] | 2′-chloro-N-(6-(4- cyclopropylpiperidin-1- yl)thiazolo[4,5- b]pyrazin-2-yl)-5′- methoxy-6-methyl- [4,4′-bipyridine]-3- carboxamide | LCMS (m/z): 536 [M + H]⁺. ¹H NMR (400 MHz, DMSO-d6) δ 12.95 (s, 1H), 8.84 (s, 1H), 8.33 (s, 1H), 8.17 (s, 1H), 7.57 (s, 1H), 7.45 (s, 1H), 4.35-4.31 (m, 2H), 3.62 (s, 3H), 2.89-2.85 (m, 2H), 2.60 (s, 3H), 1.82-1.78 (m, 2H), 1.44-1.25 (m, 2H), 0.83-0.81 (m, 1H), 0.58 (d, J = 8.6 Hz, 1H), 0.41-0.33 (m, 2H), 0.12 (q, J = 4.9 Hz, 2H) |
| 348 | 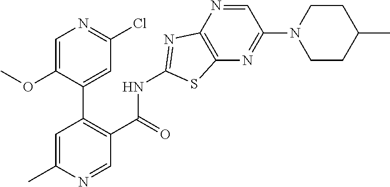 | 2′-chloro-5′-methoxy-6- methyl-N-(6-(4- methylpiperidin-1- yl)thiazolo[4,5- b]pyrazin-2-yl)-[4,4′- bipyridine]-3- carboxamide | LCMS (m/z): 510 [M + H]⁺. ¹H NMR (400 MHz, DMSO-d6) δ 12.96 (s, 1H), 8.84 (s, 1H), 8.33 (s, 1H), 8.17 (s, 1H), 7.57 (s, 1H), 7.45 (s, 1H), 4.32 (d, J = 13.0 Hz, 2H), 3.62 (s, 3H), 2.90 (dd, J = 13.5, 11.2 Hz, 2H), 2.60 (s, 3H), 1.71 (d, J = 13.4 Hz, 3H), 1.27- 1.07 (m, 2H), 0.93 (d, J = 6.4 Hz, 3H) |
| 349 | 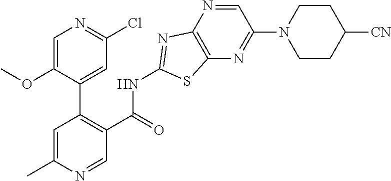 [SMILES: COc1cnc(Cl)cc1-c1cc(C)ncc1C(=O)Nc1nc2ncc(N3CCC(C#N)CC3)nc2s1] | 2′-chloro-N-(6-(4- cyanopiperidin-1- yl)thiazolo[4,5- b]pyrazin-2-yl)-5′- methoxy-6-methyl- [4,4′-bipyridine]-3- carboxamide | LCMS (m/z): 521 [M + H]⁺. ¹H NMR (400 MHz, DMSO-d₆) δ 13.00 (s, 1H), 8.84 (s, 1H), 8.39 (s, 1H), 8.17 (s, 1H), 7.58 (s, 1H), 7.46 (s, 1H), 4.76-4.70 (m, 1H), 4.04-4.01 (m, 2H), 3.63 (s, 3H), 3.47-3.41 (m, 2H), 2.60 (s, 3H), 2.10-2.00 (m, 2H), 1.79-1.71 (m, 2H) |
| 350 | 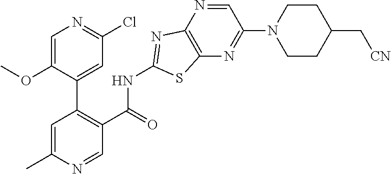 [SMILES: COc1cnc(Cl)cc1-c1cc(C)ncc1C(=O)Nc1nc2ncc(N3CCC(CC#N)CC3)nc2s1] | 2′-chloro-N-(6-(4- (cyanomethyl)piperidin- 1-yl)thiazolo[4,5- b]pyrazin-2-yl)-5′- methoxy-6-methyl- [4,4′-bipyridine]-3- carboxamide | LCMS (m/z): 535 [M + H]⁺. ¹H NMR (400 MHz, DMSO-d₆) δ 12.96 (s, 1H), 8.85 (s, 1H), 8.34 (s, 1H), 8.16 (s, 1H), 7.56 (s, 1H), 7.44 (s, 1H), 4.39-4.35 (m, 2H), 3.62 (s, 3H), 2.96-2.92 (m, 2H), 2.60 (s, 3H), 2.55 (d, J = 6.6 Hz, 2H), 1.93 (s, 1H), 1.82 (d, J = 13.0 Hz, 2H), 1.35-1.28 (m, 2H) |
| 351 | 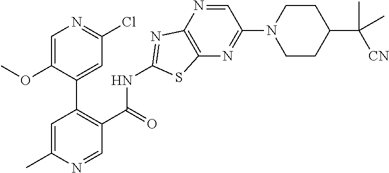 [SMILES: COc1cnc(Cl)cc1-c1cc(C)ncc1C(=O)Nc1nc2ncc(N3CCC(C(C)(C)C#N)CC3)nc2s1] | 2′-chloro-N-(6-(4-(2- cyanopropan-2- yl)piperidin-1- yl)thiazolo[4,5- b]pyrazin-2-yl)-5′- methoxy-6-methyl- [4,4′-bipyridine]-3- carboxamide | LCMS (m/z): 563 [M + H]⁺. ¹H NMR (400 MHz, DMSO-d6) δ 12.97 (s, 1H), 8.84 (s, 1H), 8.37 (s, 1H), 8.17 (s, 1H), 7.57 (s, 1H), 7.45 (s, 1H), 4.51-4.47 (m, 2H), 3.63 (s, 3H), 2.91-2.87 (m, 2H), 2.60 (s, 3H), 1.92-1.88 (m, 2H), 1.71-1.67 (m, 1H), 1.33-1.29 (m, 8H) |
| 352 | 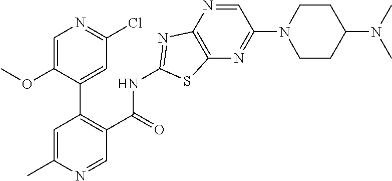 | 2′-chloro-N-(6-(4- (dimethylamino)piperidin- 1-yl)thiazolo[4,5- b]pyrazin-2-yl)-5′- methoxy-6-methyl- [4,4′-bipyridine]-3- carboxamide | LCMS (m/z): 537 [M + H]⁺. ¹H NMR (400 MHz, DMSO-d₆) δ 8.84 (s, 1H), 8.41 (s, 1H), 8.17 (s, 1H), 7.58 (s, 1H), 7.46 (s, 1H), 4.52-4.48 (m, 2H), 3.62 (s, 3H), 3.01-2.89 (m, 2H), 2.68 (s, 6H), 2.60 (s, 3H), 2.10-2.00 (m, 2H), 1.61-1.58 (m, 2H), 1.24 (s, 1H) |
| 353 | 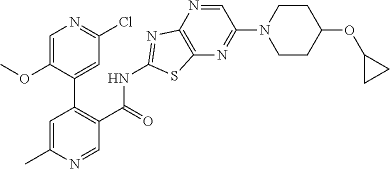 [SMILES: COc1cnc(Cl)cc1-c1cc(C)ncc1C(=O)Nc1nc2ncc(N3CCC(OC4CC4)CC3)nc2s1] | 2′-chloro-N-(6-(4- cyclopropoxypiperidin- 1-yl)thiazolo[4,5- b]pyrazin-2-yl)-5′- methoxy-6-methyl- [4,4′-bipyridine]-3- carboxamide | LCMS (m/z): 552 [M + H]⁺. ¹H NMR (300 MHz, DMSO-d6) δ 12.94 (s, 1H), 8.84 (s, 1H), 8.36 (s, 1H), 8.17 (s, 1H), 7.58 (s, 1H), 7.46 (s, 1H), 4.04-3.97 (m, 2H), 3.72-3.66 (m, 4H), 3.40-3.36 (m, 1H), 3.28 (d, J = 9.8 Hz, 2H), 2.60 (s, 3H), 2.01-1.91 (m, 2H), 1.57-1.52 (m, 2H), 0.51-0.44 (m, 4H) |
| 354 | 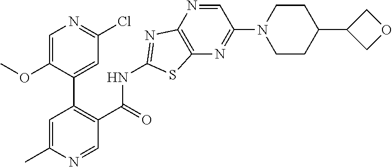 [SMILES: COc1cnc(Cl)cc1-c1cc(C)ncc1C(=O)Nc1nc2ncc(N3CCC(C4COC4)CC3)nc2s1] | 2′-chloro-5′-methoxy-6- methyl-N-(6-(4- (oxetan-3-yl)piperidin- 1-yl)thiazolo[4,5- b]pyrazin-2-yl)-[4,4′- bipyridine]-3- carboxamide | LCMS (m/z): 552 [M + H]⁺. ¹H NMR (400 MHz, DMSO-d6) δ 12.95 (s, 1H), 8.84 (s, 1H), 8.33 (s, 1H), 8.16 (s, 1H), 7.57 (s, 1H), 7.45 (s, 1H), 4.61 (dd, J = 7.9, 5.9 Hz, 2H), 4.45-4.26 (m, 4H), 3.62 (s, 3H), 3.01-2.86 (m, 2H), 2.73-2.70 (m, 1H), 2.60 (s, 3H), 1.92-1,87 (m, 1H), 1.71-1.67 (m, 2H), 1.15-0.98 (m, 2H) |
| 355 | 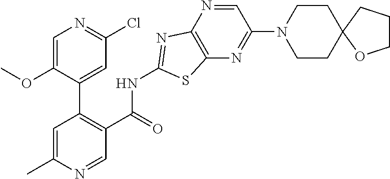 [SMILES: COc1cnc(Cl)cc1-c1cc(C)ncc1C(=O)Nc1nc2ncc(N3CCC4(CCCO4)CC3)nc2s1] | N-(6-(1-oxa-8- azaspiro[4.5]decan-8- yl)thiazolo[4,5- b]pyrazin-2-yl)-2′- chloro-5′-methoxy-6- methyl-[4,4′- bipyridine]-3- carboxamide | LCMS (m/z): 552 [M + H]⁺. ¹H NMR (400 MHz, DMSO-d₆) δ 12.96 (s, 1H), 8.83 (s, 1H), 8.36 (s, 1H), 8.17 (s, 1H), 7.58 (s, 1H), 7.46 (s, 1H), 3.89-3.71 (m, 4H), 3.62 (s, 3H), 3.42-3.36 (m, 2H), 2.60 (s, 3H), 1.93-1.86 (m, 2H), 1.70 (d, J = 8.4, 6.5 Hz, 2H), 1.62 (t, J = 5.6 Hz, 4H) |
| 356^{~} | 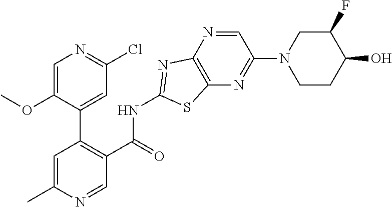 [SMILES: COc1cnc(Cl)cc1-c1cc(C)ncc1C(=O)Nc1nc2ncc(N3CC[C@H](O)[C@H](F)C3)nc2s1] | 2′-chloro-N-{6-[(3R or S,4S and R OR 3R and S,4S or R)-3-fluoro-4- hydroxypiperidin-1-yl]- [1,3]thiazolo[4,5- b]pyrazin-2-yl}-5′- methoxy-6-methyl- [4,4′-bipyridine]-3- carboxamide | LCMS (m/z): 530 [M + H]⁺. ¹H NMR (400 MHz, DMSO-d₆) δ 12.45 (s, 1H), 8.86 (s, 1H), 8.37 (s, 1H), 8.17 (s, 1H), 7.57 (s, 1H), 7.44 (s, 1H), 5.15 (d, J = 5.1 Hz, 1H), 4.90-4.52 (m, 1H), 4.37- 4.28 (m, 1H), 4.10 (d, J = 13.5 Hz, 1H), 3.89-3.78 (m, 1H), 3.63 (s, 3H), 3.52-3.38 (m, 1H), 3.27- 3.18 (m, 1H), 2.60 (s, 3H), 1.74 (d, J = 5.2 Hz, 2H) |
| 357⁺ | 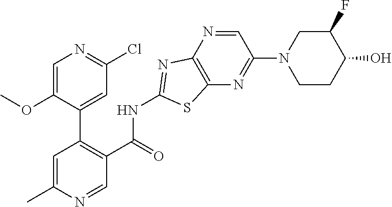 [SMILES: COc1cnc(Cl)cc1-c1cc(C)ncc1C(=O)Nc1nc2ncc(N3CC[C@@H](O)[C@H](F)C3)nc2s1] | 2′-chloro-N-(6-((3R or S,4R and S OR 3R and S,4R or S)-3-fluoro-4- hydroxypiperidin-1- yl)thiazolo[4,5- b]pyrazin-2-yl)-5′- methoxy-6-methyl- [4,4′-bipyridine]-3- carboxamide | LCMS (m/z): 530 [M + H]⁺. ¹H NMR (400 MHz, DMSO-d₆) δ 12.45 (s, 1H), 8.86 (s, 1H), 8.39 (s, 1H), 8.17 (s, 1H), 7.57 (s, 1H), 7.45 (s, 1H), 5.36 (d, J = 4.6 Hz, 1H), 4.64-4.24 (m, 1H), 4.19- 4.09 (m, 1H), 3.99-3.71 (m, 2H), 3.63 (s, 3H), 3.59-3.35 (m, 2H), 2.60 (s, 3H), 1.93 (s, 1H), 1.67- 1.29 (m, 1H) |
| 358^{$,q} | 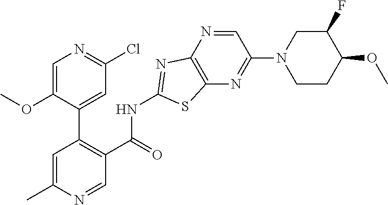 | 2′-chloro-N-(6- ((3R,4S)-3-fluoro-4- methoxypiperidin-1- yl)thiazolo[4,5- b]pyrazin-2-yl)-5′- methoxy-6-methyl- [4,4′-bipyridine]-3- carboxamide | LCMS (m/z): 544 [M + H]⁺. ¹H NMR (400 MHz, DMSO-d₆) δ 12.99 (s, 1H), 8.86 (s, 1H), 8.36 (s, 1H), 8.16 (s, 1H), 7.55 (s, 1H), 7.42 (s, 1H), 4.99 (d, J = 49.1 Hz, 1H), 4.42 (s, 1H), 4.17 (d, J = 13.5 Hz, 1H), 3.62 (s, 3H), 3.58- 3.49 (m, 1H), 3.42 (d, J = 14.5 Hz, 1H), 3.34 (s, 3H), 3.16 (t, J = 12.1 Hz, 1H), 2.59 (s, 3H), 1.90- 1.69 (m, 2H) |
| 359^{$,q} | 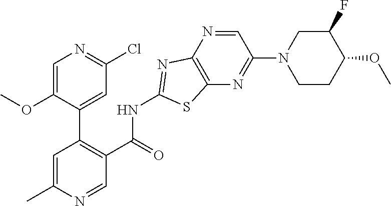 | 2′-chloro-N-(6- ((3R,4R)-3-fluoro-4- methoxypiperidin-1- yl)thiazolo[4,5- b]pyrazin-2-yl)-5′- methoxy-6-methyl- [4,4′-bipyridine]-3- carboxamide | LCMS (m/z): 544 [M + H]⁺. ¹H NMR (400 MHz, DMSO-d₆) δ 13.01 (s, 1H), 8.86 (s, 1H), 8.39 (s, 1H), 8.16 (s, 1H), 7.56 (s, 1H), 7.44 (s, 1H), 4.59 (d, J = 49.6 Hz, 1H), 4.13 (t, J = 15.1 Hz, 1H), 3.87 (d, J = 12.5 Hz, 1H), 3.62 (s, 3H), 3.55 (s, 2H), 3.39 (s, 4H), 2.60 (s, 3H), 2.05 (s, 1H), 1.73- 1.22 (m, 1H) |
| 360^{$,q} | 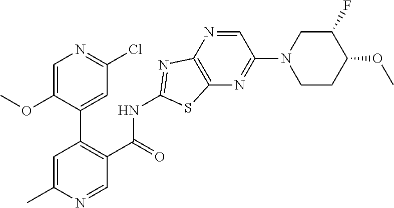 | 2′-chloro-N-(6- ((3S,4R)-3-fluoro-4- methoxypiperidin-1- yl)thiazolo[4,5- b]pyrazin-2-yl)-5′- methoxy-6-methyl- [4,4′-bipyridine]-3- carboxamide | LCMS (m/z): 544 [M + H]⁺. ¹H NMR (400 MHz, DMSO-d₆) δ 13.01 (s, 1H), 8.86 (s, 1H), 8.39 (s, 1H), 8.16 (s, 1H), 7.56 (s, 1H), 7.44 (s, 1H), 4.53 (s, 1H), 4.13 (s, 1H), 3.87 (s, 1H), 3.62 (s, 3H), 3.55 (s, 2H), 3.39 (s, 4H), 2.59 (s, 3H), 2.06 (s, 1H), 1.55 (s, 1H) |
| 361^{$,q} | 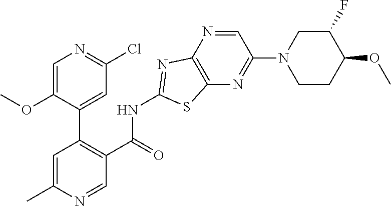 | 2′-chloro-N-(6- ((3S,4S)-3-fluoro-4- methoxypiperidin-1- yl)thiazolo[4,5- b]pyrazin-2-yl)-5′- methoxy-6-methyl- [4,4′-bipyridine]-3- carboxamide | LCMS (m/z): 544 [M + H]⁺. ¹H NMR (400 MHz, DMSO-d₆) δ 12.98 (s, 1H), 8.86 (s, 1H), 8.36 (s, 1H), 8.16 (s, 1H), 7.56 (s, 1H), 7.43 (s, 1H), 4.99 (d, J = 49.1 Hz, 1H), 4.42 (s, 1H), 4.16 (s, 1H), 3.62 (s, 3H), 3.54-3.41 (m, 1H), 3.34 (s, 3H), 3.16 (s, 1H), 2.67 (s, 1H), 2.59 (s, 3H), 1.90-1.69 (m, 2H) |
| 362^{∧} | 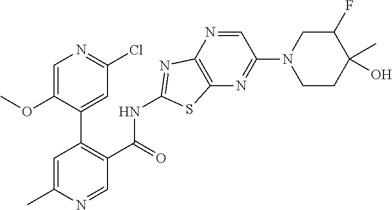 [SMILES: COc1cnc(Cl)cc1-c1cc(C)ncc1C(=O)Nc1nc2ncc(N3CCC(C)(O)C(F)C3)nc2s1] | 2′-chloro-N-[6-(3- fluoro-4-hydroxy-4- methylpiperidin-1-yl)- [11,3] thiazolo[4,5-b] pyrazin-2-yl]-5′- methoxy-6-methyl- [4,4′-bipyridine]-3- carboxamide | LCMS (m/z): 544 [M + H]⁺. ¹H NMR (300 MHz, DMSO-d₆) δ 12.99 (s, 1H), 8.84 (s, 1H), 8.39 (s, 1H), 8.17 (s, 1H), 7.58 (s, 1H), 7.46 (s, 1H), 4.86 (s, 1H), 4.51- 4.41 (m, 1H), 4.36-4.25 (m, 1H), 4.18-4.03 (m, 1H), 3.86-3.76 (m, 1H), 3.62 (s, 3H), 3.60-3.42 (m, 2H), 2.60 (s, 3H), 1.72 (s, 1H), 1.57 (s, 1H), 1.24 (s, 3H) |
| 363^{r} | 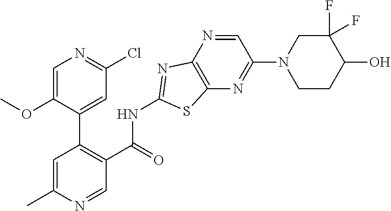 [SMILES: COc1cnc(Cl)cc1-c1cc(C)ncc1C(=O)Nc1nc2ncc(N3CCC(O)C(F)(F)C3)nc2s1] | (S or R)-2′-chloro-N-(6- (3,3-difluoro-4- hydroxypiperidin-1- yl)thiazolo[4,5- b]pyrazin-2-yl)-5′- methoxy-6-methyl- [4,4′-bipyridine]-3- carboxamide | LCMS (m/z): 548 [M + H]⁺. ¹H NMR (400 MHz, DMSO-d₆) δ 8.95 (s, 1H), 8.28 (s, 1H), 8.14 (s, 1H), 7.46 (s, 1H), 7.31 (s, 1H), 5.73 (d, J = 5.4 Hz, 1H), 4.13- 4.03 (m, 1H), 3.91 (s, 1H), 3.81- 3.64 (m, 2H), 3.63 (s, 3H), 3.50 (s, 1H), 2.57 (d, J = 10.6 Hz, 3H), 1.94 (d, J = 65.7 Hz, 2H) |
| 364^{r} | 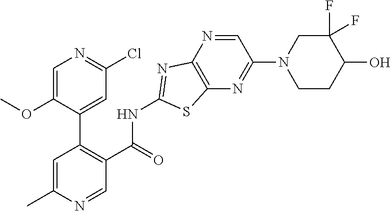 [SMILES: COc1cnc(Cl)cc1-c1cc(C)ncc1C(=O)Nc1nc2ncc(N3CCC(O)C(F)(F)C3)nc2s1] | (R or S)-2′-chloro-N-(6- (3,3-difluoro-4- hydroxypiperidin-1- yl)thiazolo[4,5- b]pyrazin-2-yl)-5′- methoxy-6-methyl- [4,4′-bipyridine]-3- carboxamide | LCMS (m/z): 548 [M + H]⁺. ¹H NMR (400 MHz, DMSO-d₆) δ 8.95 (s, 1H), 8.29 (s, 1H), 8.14 (s, 1H), 7.46 (s, 1H), 7.31 (s, 1H), 5.72 (s, 1H), 4.08 (s, 1H), 3.91 (d, J = 16.2 Hz, 1H), 3.81 (s, 2H), 3.63 (s, 3H), 3.51 (d, J = 4.5 Hz, 1H), 2.58 (s, 3H), 1.92 (d, J = 1.9 Hz, 1H), 1.78-1.75 (m, 1H) |
| 365 | 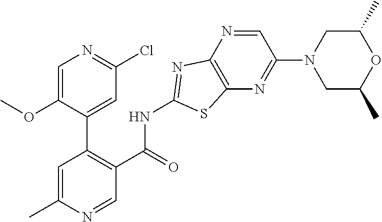 | 2′-chloro-N-(6- ((2S,6S)-2,6- dimethylmorpholino) thiazolo[4,5-b]pyrazin-2- yl)-5′-methoxy-6- methyl-[4,4′- bipyridine]-3- carboxamide | LCMS (m/z): 526 [M + H]⁺. ¹H NMR (400 MHz, Chloroform-d) δ 11.95 (s, 1H), 8.86 (s, 1H), 8.04 (s, 1H), 7.76 (s, 1H), 7.09 (d, J = 21.1 Hz, 2H), 4.27-4.01 (m, 2H), 3.70 (d, J = 15.4 Hz, 5H), 3.28 (dd, J = 12.7, 6.4 Hz, 2H), 2.64 (s, 3H), 1.29 (d, J = 6.5 Hz, 6H) |
| 366 | 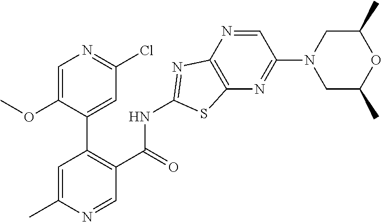 | 2′-chloro-N-(6- ((2S,6R)-2,6- dimethylmorpholino) thiazolo[4,5-b]pyrazin- 2-yl)-5′-methoxy-6- methyl-[4,4′- bipyridine]-3- carboxamide | LCMS (m/z): 526 [M + H]⁺. ¹H NMR (300 MHz, Chloroform-d) δ 10.90 (s, 1H), 8.91 (s, 1H), 8.07 (s, 1H), 7.94 (s, 1H), 7.26-7.14 (m, 2H), 4.11 (d, J = 12.7 Hz, 2H), 3.78 (s, 5H), 2.70 (s, 5H), 1.32 (d, J = 6.5 Hz, 6H) |
| 367 | 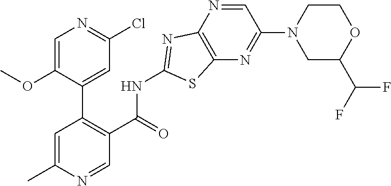 [SMILES: COc1cnc(Cl)cc1-c1cc(C)ncc1C(=O)Nc1nc2ncc(N3CCOC(C(F)F)C3)nc2s1] | 2′-chloro-N-(6-(2- (difluoromethyl) morpholino)thiazolo [4,5-b]pyrazin-2-yl)-5′- methoxy-6-methyl- [4,4′-bipyridine]-3- carboxamide | LCMS (m/z): 548 [M + H]⁺. ¹H NMR (400 MHz, DMSO-d₆) δ 13.00 (brs, 1H), 8.86 (s, 1H), 8.38 (s, 1H), 8.16 (s, 1H), 7.56 (s, 1H), 7.44 (s, 1H), 6.16 (m, 1H), 4.30- 4.22 (m, 1H), 4.15-4.02 (m, 2H), 3.97-3.84 (m, 1H), 3.69 (td, J = 11.5, 2.7 Hz, 1H), 3.62 (s, 3H), 3.25 (s, 1H), 3.06 (td, J = 12.3, 3.4 Hz, 1H), 2.93 (dd, J = 12.8, 10.7 Hz, 1H), 2.60 (s, 3H) |
| 368 | 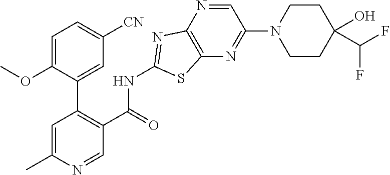 | 4-(5-cyano-2- methoxyphenyl)-N-(6- (4-(difluoromethyl)-4- hydroxypiperidin-1- yl)thiazolo[4,5- b]pyrazin-2-yl)-6- methylnicotinamide | LCMS (m/z): 522 [M + H]⁺. ¹H NMR (400 MHz, DMSO-d₆) δ 12.86 (s, 1H), 8.77 (s, 1H), 8.36 (s, 1H), 7.94-7.84 (m, 2H), 7.41 (s, 1H), 7.17 (d, J = 8.6 Hz, 1H), 5.74 (t, J = 56.0 Hz, 1H), 5.40 (s, 1H), 4.25 (d, J = 13.0 Hz, 2H), 3.59 (s, 3H), 3.26-3.14 (m, 2H), 2.59 (s, 3H), 1.65-1.55 (m, 4H) |
| 369 | 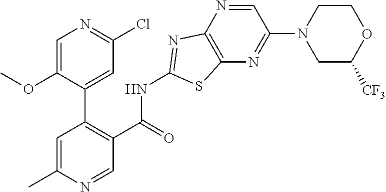 [SMILES: COc1cnc(Cl)cc1-c1cc(C)ncc1C(=O)Nc1nc2ncc(N3CCO[C@H](C(F)(F)F)C3)nc2s1] | (S)-2′-chloro-5′- methoxy-6-methyl-N- (6-(2- (trifluoromethyl) morpholino)thiazolo [4,5-b]pyrazin-2-yl)- [4,4′-bipyridine]-3- carboxamide | LCMS (m/z): 566 [M + H]⁺. ¹H NMR (400 MHz, DMSO-d₆) δ 13.06 (s, 1H), 8.86 (s, 1H), 8.43 (s, 1H), 8.16 (s, 1H), 7.57 (s, 1H), 7.45 (s, 1H), 4.38 (d, J = 11.8 Hz, 2H), 4.20-4.07 (m, 2H), 3.80- 3.72 (m, 1H), 3.62 (s, 3H), 3.07 (dt, J = 31.3, 11.6 Hz, 2H), 2.60 (s, 3H) |

^{~}Mixture of cis or trans isomers
⁺Mixture of trans or cis isomers
^{∧}Racemic
^{$}Single unknown isomer
^{p}The racemic product was separated by prep-chiral-HPLC with the following conditions to isolate Example 339 and 340: (Column: CHIRALPAK IC-3, 4.6 × 50 mm, 3 μm; Mobile Phase A: Hex/MTBE = 1/1) (0.1% TFA): EtOH = 80:20; Gradient: isocratic); first eluting peak 1.90 min (Example 339), second eluting peak 2.77 min (Example 340)
^{q}Chiral separation conditions for Examples 358-361: The racemic product (34.2 mg) was separated by prep-chiral-HPLC with the following conditions (Column: CHIRALPAK IA-3 4.6 × 100 mm, 3 μm; Mobile Phase A: (MTBE: Hex = 1:1)(0.1% FA):EtOH = 70:30; Flow rate: 1.67 mL/min; Gradient: 0% B to 0% B; Injection Volume: 3 mL; Retention times: 1.73 min (Example 358), 2.19 min (Example 359), 1.39 min (Example 360), 1.69 min (Example 361).
^{r}The racemic product was separated by prep-CHIRAL-HPLC with the following conditions to isolate Example 363 and 364: (Column: CHIRALPAK ID-3, 4.6 × 50 mm, 3 μm; Mobile Phase A: MTBE (0.1% FA): (MeOH: DCM = 1:1) = 85: 15; Flow rate: 1.0 mL/min; Gradient: isocratic); Retention time: 2.43 min (Example 363), 3.25 min (Example 364).

### Example 370: Synthesis of 2′-chloro-5′-methoxy-6-methyl-N-(6-(4-(methylamino)piperidin-1-yl)thiazolo[4,5-b]pyrazin-2-yl)-[4,4′-bipyridine]-3-carboxamide

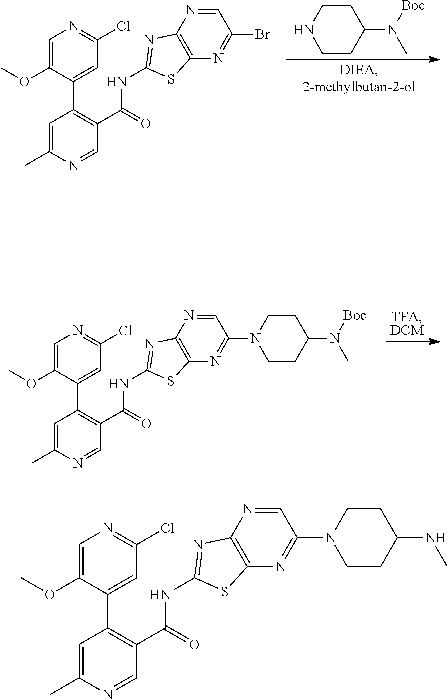

Step 1: Completed as described in Example 326 to afford tert-butyl (1-(2-(2′-chloro-5′-methoxy-6-methyl-[4,4′-bipyridine]-3-carboxamido)thiazolo[4,5-b]pyrazin-6-yl)piperidin-4-yl)(methyl)carbamate (150 mg, 39% yield) as a yellow solid. LCMS (ESI, m/z): 625 [M+H]⁺.

Step 2: To a solution of tert-butyl (1-(2-(2′-chloro-5′-methoxy-6-methyl-[4,4′-bipyridine]-3-carboxamido)thiazolo[4,5-b]pyrazin-6-yl)piperidin-4-yl)(methyl)carbamate (140 mg, 0.22 mmol) in DCM (3 mL) was added TFA (1.1 g, 9.2 mmol) dropwise at 25° C. The resulting mixture was stirred at 25° C. for 1 h, and then concentrated under reduced pressure. The residue was purified by reverse-phase flash chromatography (10-50% MeCN in water with 0.1% formic acid modifier) to afford 2′-chloro-5′-methoxy-6-methyl-N-(6-(4-(methylamino)piperidin-1-yl)thiazolo[4,5-b]pyrazin-2-yl)-[4,4′-bipyridine]-3-carboxamide (85 mg, 70% yield) as a yellow solid. LCMS (ESI, m/z): 523 [M−H]⁻. ¹H NMR (400 MHz, DMSO-d₆) δ 9.21 (d, J=2.7 Hz, 1H), 8.44 (d, J=3.2 Hz, 1H), 8.29 (d, J=2.6 Hz, 1H), 8.18 (s, 1H), 7.74 (s, 1H), 4.45 (d, J=13.5 Hz, 2H), 3.66 (d, J=2.2 Hz, 3H), 3.36-3.24 (m, 1H), 3.02 (t, J=12.7 Hz, 2H), 2.89 (s, 3H), 2.56 (s, 3H), 2.16 (d, J=11.9 Hz, 2H), 1.63 (d, J=12.2 Hz, 2H).

TABLE AV
The following example was prepared using a similar procedure to that described for
Example 370

| Ex. | | | |
|---|---|---|---|
| No. | Structure | Compound Name | NMR |
| 371 | 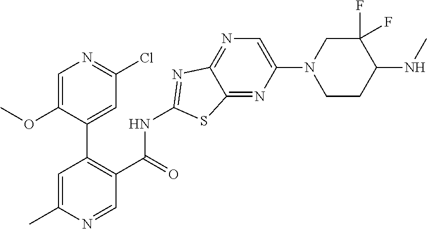 | 2′-chloro-N-(6-(3,3- difluoro-4- (methylamino)piperidin- 1-yl)thiazolo[4,5- b]pyrazin-2-yl)-5′- methoxy-6-methyl- [4,4′-bipyridine]-3- carboxamide | LCMS (m/z): 561 [M + H]⁺. ¹H NMR (400 MHz, DMSO-d₆) δ 8.84 (s, 1H), 8.45 (s, 1H), 8.15 (d, J = 11.8 Hz, 1H), 7.58 (s, 1H), 7.46 (s, 1H), 4.41-4.23 (m, 1H), 4.02 (d, J = 13.8 Hz, 1H), 3.62 (s, 3H), 3.51-3.38 (m, 2H), 3.00 (s, 1H), 2.60 (s, 3H), 2.54 (s, 1H), 2.41 (s, 3H), 1.99 (s, 1H), 1.62 (d, J = 11.3 Hz, 1H) |

### Example 372: Synthesis of 2′-chloro-N-(6-(3-(hydroxymethyl)azetidin-1-yl)thiazolo[4,5-b]pyrazin-2-yl)-5′-methoxy-6-methyl-[4,4′-bipyridine]-3-carboxamide

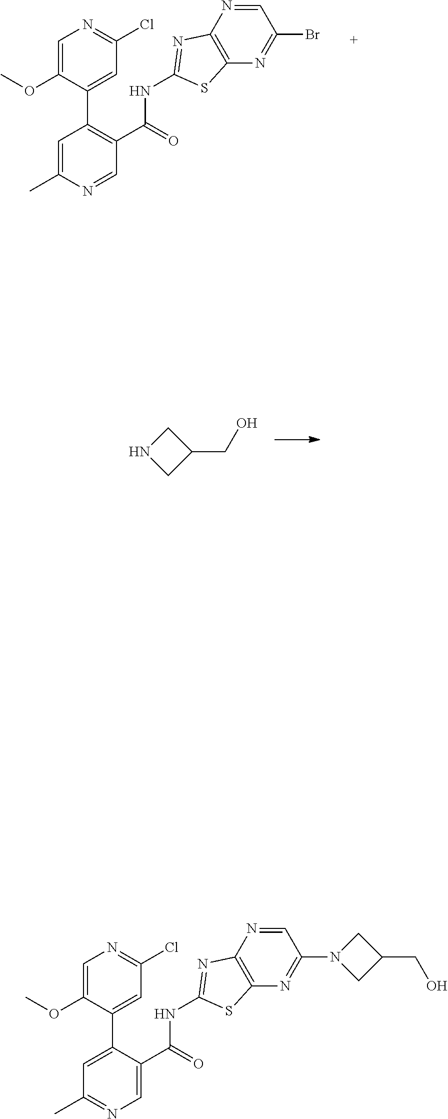 [SMILES: COc1cnc(Cl)cc1-c1cc(C)ncc1C(=O)Nc1nc2ncc(Br)nc2s1 ; OCC1CNC1 ; COc1cnc(Cl)cc1-c1cc(C)ncc1C(=O)Nc1nc2ncc(N3CC(CO)C3)nc2s1]

To a solution of N-(6-bromothiazolo[4,5-b]pyrazin-2-yl)-2′-chloro-5′-methoxy-6-methyl-[4,4′-bipyridine]-3-carboxamide (300 mg, 0.6 mmol) in DMSO (2 mL) was added DBU (279 mg, 1.8 mmol) and azetidin-3-ylmethanol (159 mg, 1.8 mmol) in one portion at 25° C. The reaction mixture was charged with N₂ three times at 25° C. The reaction mixture was stirred at 100° C. for 16 h under a nitrogen atmosphere. The resulting mixture was cooled to 25° C. and purified directly by reverse-phase flash chromatography (20-60% MeCN in water with 0.1% formic acid modifier) to afford 2′-chloro-N-(6-(3-(hydroxymethyl)azetidin-1-yl)thiazolo[4,5-b]pyrazin-2-yl)-5′-methoxy-6-methyl-[4,4′-bipyridine]-3-carboxamide (79 mg, 26% yield) as a yellow solid. LCMS (ESI, m/z): 498 [M+H]⁺, ¹H NMR (400 MHz, DMSO-d₆) δ 12.95 (s, 1H), 8.84 (s, 1H), 8.16 (s, 1H), 7.84 (s, 1H), 7.56 (s, 1H), 7.44 (s, 1H), 4.81 (t, J=5.3 Hz, 1H), 4.10 (t, J=8.2 Hz, 2H), 3.83 (d, J=5.5 Hz, 2H), 3.64-3.58 (m, 5H), 2.89-2.83 (m, 1H), 2.60 (s, 3H).

TABLE AW
The following examples were prepared using a similar procedure to that described for
Example 372

| Ex. | | | |
|---|---|---|---|
| No. | Structure | Compound Name | Characterization Data |
| 373 | 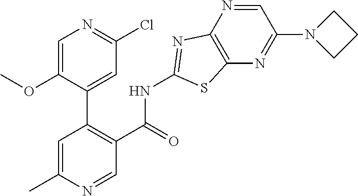 [SMILES: COc1cnc(Cl)cc1-c1cc(C)ncc1C(=O)Nc1nc2ncc(N3CCC3)nc2s1] | N-(6-(azetidin-1- yl)thiazolo[4,5-b]pyrazin- 2-yl)-2′-chloro-5′- methoxy-6-methyl-[4,4′- bipyridine]-3- carboxamide | LCMS (m/z): 468 [M + H]⁺. ¹H NMR (400 MHz, DMSO-d₆) δ 12.96 (s, 1H), 8.84 (s, 1H), 8.16 (s, 1H), 7.83 (s, 1H), 7.56 (s, 1H), 7.44 (s, 1H), 4.09 (t, J = 7.4 Hz, 4H), 3.62 (s, 3H), 2.60 (s, 3H), 2.39 (p, J = 7.4 Hz, 2H) |

### Example 374A & 374B: Synthesis of 2′-chloro-N-(6-((3R or S,4S or R)-3-fluoro-4-hydroxy-4-methylpiperidin-1-yl)thiazolo[4,5-b]pyrazin-2-yl)-5′-methoxy-6-methyl-[4,4′-bipyridine]-3-carboxamide (374A) & 2′-chloro-N-(6-((3S or R,4R or S)-3-fluoro-4-hydroxy-4-methylpiperidin-1-yl)thiazolo[4,5-b]pyrazin-2-yl)-5′-methoxy-6-methyl-[4,4′-bipyridine]-3-carboxamide (374B) with arbitrarily assigned stereochemistry

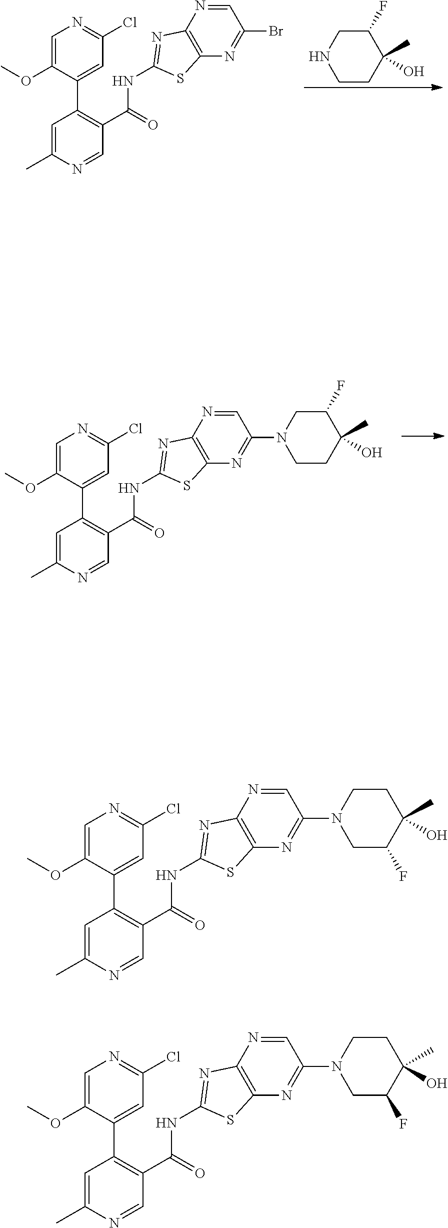

Step 1: To a stirred solution of N-(6-bromothiazolo[4,5-b]pyrazin-2-yl)-2′-chloro-5′-methoxy-6-methyl-[4,4′-bipyridine]-3-carboxamide (400 mg, 0.83 mmol) and 3-fluoro-4-methylpiperidin-4-ol (mixture of cis isomers 162 mg, 1.22 mmol) in 1,4-dioxane (15 mL) were added sodium 2-methylpropan-2-olate (235 mg, 2.44 mmol) and (SP-4-1)-[1,3-BIs[2,6-bis(1-ethylpropyl)phenyl]-4,5-dichloro-1,3-dihydro-2H-imidazol-2-ylidene]dichloro(2-methylpyridine)palladium (68 mg, 0.08 mmol) at room temperature under a nitrogen atmosphere. The resulting mixture was stirred for 16 h at 60° C. under a nitrogen atmosphere. The residue was purified by silica gel column chromatography, eluting with CH₂Cl₂/MeOH (0-10% gradient) to afford 2′-chloro-N-(6-((3S,4R)-3-fluoro-4-hydroxy-4-methylpiperidin-1-yl)thiazolo[4,5-b]pyrazin-2-yl)-5′-methoxy-6-methyl-[4,4′-bipyridine]-3-carboxamide (90 mg, 20% yield) as yellow solid. LCMS (ESI, m/z): 544 [M+H]⁺.

Step 2: The mixture of cis isomers was separated by prep-SFC (CHIRAL ART Amylose-SA, 3×25 cm, 5 μm column, 55%/45% 1:1 IPA/DCM (with 20 mM NH₃ modifier)/CO₂ at 100 min/mL) to afford two peaks:

Peak 1 RT=6.27 min: 2′-chloro-N-(6-((3R or S,4S or R)-3-fluoro-4-hydroxy-4-methylpiperidin-1-yl)thiazolo[4,5-b]pyrazin-2-yl)-5′-methoxy-6-methyl-[4,4′-bipyridine]-3-carboxamide (37 mg, 90% purity).

Peak 2 RT=7.55 min) 2′-chloro-N-(6-((3S or R,4R or S)-3-fluoro-4-hydroxy-4-methylpiperidin-1-yl)thiazolo[4,5-b]pyrazin-2-yl)-5′-methoxy-6-methyl-[4,4′-bipyridine]-3-carboxamide (25 mg, 80% purity).

A second round of purification was performed as follows:

Crude 2′-chloro-N-(6-((3R or S,4S or R)-3-fluoro-4-hydroxy-4-methylpiperidin-1-yl)thiazolo[4,5-b]pyrazin-2-yl)-5′-methoxy-6-methyl-[4,4′-bipyridine]-3-carboxamide (37 mg, 90% purity) was purified by reverse phase column chromatography (36-54% MeOH in water with 10 mmol/L NH₄HCO_{3+0.05}% NH₃H₂O modifier) to afford 2′-chloro-N-(6-((3R or S,4S or R)-3-fluoro-4-hydroxy-4-methylpiperidin-1-yl)thiazolo[4,5-b]pyrazin-2-yl)-5′-methoxy-6-methyl-[4,4′-bipyridine]-3-carboxamide (14.8 mg, 16%) as yellow solid. LCMS (ESI, m/z): 544 [M+H]⁺. ¹H NMR (400 MHz, DMSO-d₆): δ 12.96 (s, 1H), 8.87 (s, 1H), 8.35 (s, 1H), 8.16 (s, 1H), 7.54 (s, 1H), 7.42 (s, 1H), 4.85 (s, 1H), 4.46-4.28 (m, 1H), 4.18-4.02 (m, 1H), 3.88-3.74 (m, 1H), 3.62 (s, 3H), 3.58-3.50 (m, 1H), 3.44 (d, J=10.9 Hz, 1H), 2.59 (s, 3H), 1.74 (d, J=14.0 Hz, 1H), 1.63-1.52 (m, 1H), 1.23 (s, 3H).

Crude 2′-chloro-N-(6-((3S or R,4R or S)-3-fluoro-4-hydroxy-4-methylpiperidin-1-yl)thiazolo[4,5-b]pyrazin-2-yl)-5′-methoxy-6-methyl-[4,4′-bipyridine]-3-carboxamide (25 mg, 80% purity) was purified by reverse phase column chromatography (38-55% MeOH in water with 10 mmol/L NH₄HCO_{3+0.05}% NH₃H₂O modifier) to afford 2′-chloro-N-(6-((3S or R,4R or S)-3-fluoro-4-hydroxy-4-methylpiperidin-1-yl)thiazolo[4,5-b]pyrazin-2-yl)-5′-methoxy-6-methyl-[4,4′-bipyridine]-3-carboxamide (8.8 mg, 9%) as yellow solid. LCMS (ESI, m/z): 544 [M+H]⁺. ¹H NMR (400 MHz, DMSO-d₆): δ 12.96 (s, 1H), 8.87 (s, 1H), 8.35 (s, 1H), 8.16 (s, 1H), 7.54 (s, 1H), 7.42 (s, 1H), 4.85 (s, 1H), 4.46-4.28 (m, 1H), 4.18-4.02 (m, 1H), 3.88-3.74 (m, 1H), 3.62 (s, 3H), 3.58-3.50 (m, 1H), 3.44 (d, J=10.9 Hz, 1H), 2.59 (s, 3H), 1.74 (d, J=14.0 Hz, 1H), 1.63-1.52 (m, 1H), 1.23 (s, 3H).

TABLE AX
The following examples were prepared using a similar procedure to that described
for Example 374A

| Ex. | | | |
|---|---|---|---|
| No. | Structure | Compound Name | Characterization Data |
| 375 | 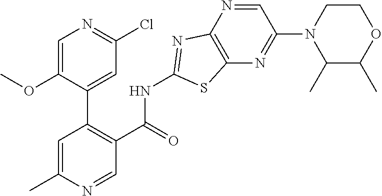 | 2′-chloro-N-(6-(2,3- dimethylmorpholino) thiazolo[4,5-b]pyrazin-2- yl)-5′-methoxy-6- methyl-[4,4′- bipyridine]-3- carboxamide | LCMS (m/z): 526 [M + H]⁺. ¹H NMR (300 MHz, DMSO-d₆) δ 12.92 (br, 1H) δ 8.85 (s, 1H), 8.29 (s, 1H), 8.17 (s, 1H), 7.58 (s, 1H), 7.46 (s, 1H), 4.39-4.10 (m, 1H), 4.08-3.75 (m, 3H), 3.78- 3.55 (m, 5H), 2.60 (s, 3H), 1.40- 0.95 (m, 6H) |
| 376^{&,s} | 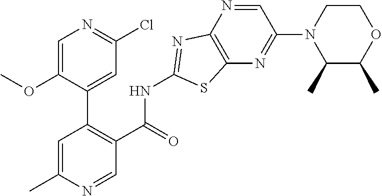 | 2′-chloro-N-(6-((2S or R,3R or S)-2,3- dimethylmorpholino) thiazolo[4,5-b]pyrazin-2- yl)-5′-methoxy-6- methyl-[4,4′- bipyridine]-3- carboxamide | LCMS (m/z): 526 [M + H]⁺. ¹H NMR (300 MHz, DMSO-d₆) δ 12.92 (br, 1H) δ 8.85 (s, 1H), 8.29 (s, 1H), 8.17 (s, 1H), 7.58 (s, 1H), 7.46 (s, 1H), 4.39-4.10 (m, 1H), 4.08-3.75 (m, 3H), 3.78- 3.55 (m, 5H), 2.60 (s, 3H), 1.40- 0.95 (m, 6H) |
| 377^{&,s} | 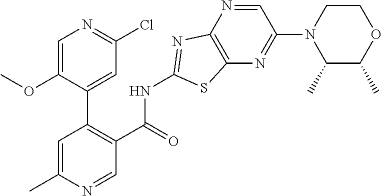 | 2′-chloro-N-(6-((2R or S,3S or R)-2,3- dimethylmorpholino) thiazolo[4,5-b]pyrazin-2- yl)-5′-methoxy-6- methyl-[4,4′- bipyridine]-3- carboxamide | LCMS (m/z): 526 [M + H]⁺. ¹H NMR (300 MHz, DMSO-d₆) δ 12.92 (br, 1H) δ 8.85 (s, 1H), 8.29 (s, 1H), 8.17 (s, 1H), 7.58 (s, 1H), 7.46 (s, 1H), 4.39-4.10 (m, 1H), 4.08-3.75 (m, 3H), 3.78- 3.55 (m, 5H), 2.60 (s, 3H), 1.40- 0.95 (m, 6H) |
| 378^{&,s} | 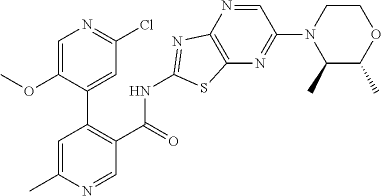 | 2′-chloro-N-(6-((2R or S,3R or S)-2,3- dimethylmorpholino) thiazolo[4,5-b]pyrazin-2- yl)-5′-methoxy-6- methyl-[4,4′- bipyridine]-3- carboxamide | LCMS (m/z): 526 [M + H]⁺. ¹H NMR (300 MHz, DMSO-d₆) δ 12.92 (br, 1H) δ 8.85 (s, 1H), 8.29 (s, 1H), 8.17 (s, 1H), 7.58 (s, 1H), 7.46 (s, 1H), 4.39-4.10 (m, 1H), 4.08-3.75 (m, 3H), 3.78- 3.55 (m, 5H), 2.60 (s, 3H), 1.40- 0.95 (m, 6H) |
| 379^{&,s} | 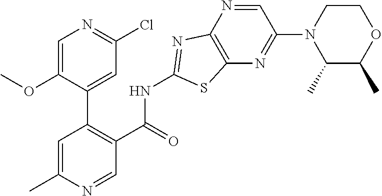 | 2′-chloro-N-(6-((2S or R,3S or R)-2,3- dimethylmorpholino) thiazolo[4,5-b]pyrazin-2- yl)-5′-methoxy-6- methyl-[4,4′- bipyridine]-3- carboxamide | LCMS (m/z): 526 [M + H]⁺. ¹H NMR (300 MHz, DMSO-d₆) δ 12.92 (br, 1H) δ 8.85 (s, 1H), 8.29 (s, 1H), 8.17 (s, 1H), 7.58 (s, 1H), 7.46 (s, 1H), 4.39-4.10 (m, 1H), 4.08-3.75 (m, 3H), 3.78- 3.55 (m, 5H), 2.60 (s, 3H), 1.40- 0.95 (m, 6H) |
| 380 | 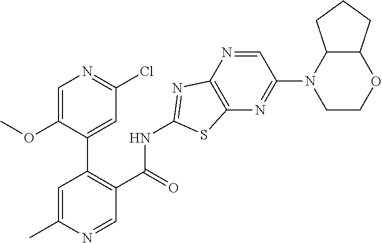 [SMILES: COc1cnc(Cl)cc1-c1cc(C)ncc1C(=O)Nc1nc2ncc(N3CCOC4CCCC43)nc2s1] | 2′-chloro-N-(6- (hexahydrocyclopenta [b][1,4]oxazin-4(4aH)- yl)thiazolo[4,5-b] pyrazin-2-yl)-5′- methoxy-6-methyl- [4,4′-bipyridine]-3- carboxamide | LCMS (m/z): 538 [M + H]⁺. ¹H NMR (300 MHz, DMSO-d₆): δ 13.14 (s, 1H), 8.87 (s, 1H), 8.40 (s, 1H), 8.16 (s, 1H), 7.57 (s, 1H), 7.44 (s, 1H), 4.03-3.97 (m, 1H), 3.89-3.76 (m, 2H), 3.61 (s, 3H), 3.48-3.38 (m, 2H), 2.99-2.82 (m, 2H), 2.60 (s, 3H), 1.86 (d, J = 7.9 Hz, 1H), 1.73-1.63 (m, 2H), 1.56-1.42 (m, 2H) |
| 381 | 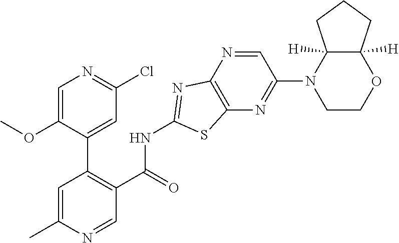 [SMILES: COc1cnc(Cl)cc1-c1cc(C)ncc1C(=O)Nc1nc2ncc(N3CCO[C@@H]4CCC[C@@H]43)nc2s1] | 2′-chloro-N-(6- ((4aS,7aR)- hexahydrocyclopenta [b][1,4]oxazin-4(4aH)- yl)thiazolo[4,5- b]pyrazin-2-yl)-5′- methoxy-6-methyl- [4,4′-bipyridine]-3- carboxamide | LCMS (m/z): 538 [M + H]⁺. ¹H NMR (300 MHz, DMSO-d₆): δ 13.00 (s, 1H), 8.85 (s, 1H), 8.29 (s, 1H), 8.16 (s, 1H), 7.57 (s, 1H), 7.45 (s, 1H), 4.41-4.35 (m, 1H), 3.97-3.86 (m, 3H), 3.62 (s, 3H), 3.53 (t, J = 11.7 Hz, 1H), 3.07 (t, J = 11.4 Hz, 1H), 2.60 (s, 3H), 1.85 (d, J = 7.4 Hz, 3H), 1.78- 1.66 (m, 3H) |
| 382 | 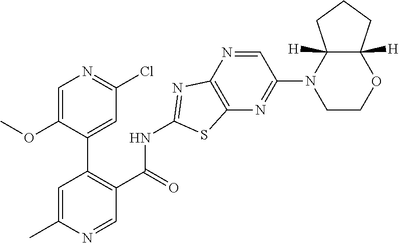 [SMILES: COc1cnc(Cl)cc1-c1cc(C)ncc1C(=O)Nc1nc2ncc(N3CCO[C@H]4CCC[C@H]43)nc2s1] | 2′-chloro-N-(6- ((4aR,7aS)- hexahydrocyclopenta [b][1,4]oxazin-4(4aH)- yl)thiazolo[4,5- b]pyrazin-2-yl)-5′- methoxy-6-methyl- [4,4′-bipyridine]-3- carboxamide | LCMS (m/z): 538 [M + H]⁺. ¹H NMR (400 MHz, DMSO-d₆): δ 12.98 (s, 1H), 8.85 (s, 1H), 8.29 (s, 1H), 8.16 (s, 1H), 7.57 (s, 1H), 7.45 (s, 1H), 4.39-4.35 (m, 1H), 3.98-3.83 (m, 3H), 3.62 (s, 3H), 3.54 (t, J = 11.7 Hz, 1H), 3.07 (t, J = 11.4 Hz, 1H), 2.60 (s, 3H), 1.84 (d, J = 7.4 Hz, 3H), 1.70- 1.58 (m, 3H) |
| 383 | 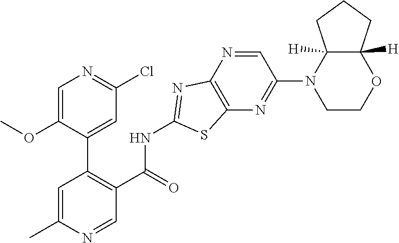 [SMILES: COc1cnc(Cl)cc1-c1cc(C)ncc1C(=O)Nc1nc2ncc(N3CCO[C@H]4CCC[C@@H]43)nc2s1] | 2′-chloro-N-(6- ((4aS,7aS)- hexahydrocyclopenta [b][1,4]oxazin-4(4aH)- yl)thiazolo[4,5- b]pyrazin-2-yl)-5′- methoxy-6-methyl- [4,4′-bipyridine]-3- carboxamide | LCMS (m/z): 538 [M + H]⁺. ¹H NMR (400 MHz, DMSO-d₆) δ 13.12 (s, 1H), 8.86 (s, 1H), 8.41 (s, 1H), 8.17 (s, 1H), 7.57 (s, 1H), 7.45 (s, 1H), 4.02-3.99 (m, 1H), 3.88 (d, J = 12.8 Hz, 1H), 3.78 (m, 1H), 3.63 (s, 3H), 3.47-3.40 (m, 1H), 2.98 (m, 1H), 2.89- 2.80 (m, 1H), 2.61 (s, 3H), 2.55 (d, J = 4.3 Hz, 1H), 1.86 (m, 1H), 1.74-1.60 (m, 2H), 1.56-1.41 (m, 2H) |
| 384 | 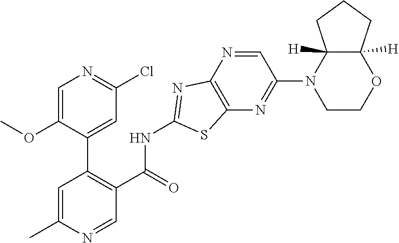 [SMILES: COc1cnc(Cl)cc1-c1cc(C)ncc1C(=O)Nc1nc2ncc(N3CCO[C@@H]4CCC[C@H]43)nc2s1] | 2′-chloro-N-(6- ((4aR,7aR)- hexahydrocyclopenta [b][1,4]oxazin-4(4aH)- yl)thiazolo[4,5- b]pyrazin-2-yl)-5′- methoxy-6-methyl- [4,4′-bipyridine]-3- carboxamide | LCMS (m/z): 538 [M + H]⁺. ¹H NMR (400 MHz, DMSO-d₆): δ 13.11 (s, 1H), 8.91 (s, 1H), 8.33 (s, 1H), 8.15 (s, 1H), 7.52 (s, 1H), 7.38 (s, 1H), 4.04-3.97 (m, 1H), 3.85-3.73 (m, 2H), 3.62 (s, 3H), 3.48-3.38 (m, 1H), 3.00-2.91 (m, 1H), 2.86-2.78 (m, 1H), 2.59 (s, 3H), 1.85 (t, J = 9.9 Hz, 1H), 1.78-1.37 (m, 5H) |
| 385 | 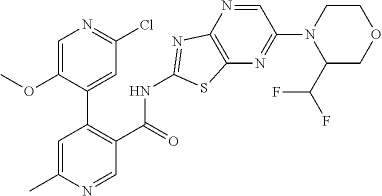 [SMILES: COc1cnc(Cl)cc1-c1cc(C)ncc1C(=O)Nc1nc2ncc(N3CCOCC3C(F)F)nc2s1] | 2′-chloro-N-(6-(3- (difluoromethyl) morpholino)thiazolo [4,5-b]pyrazin-2-yl)-5′- methoxy-6-methyl- [4,4′-bipyridine]-3- carboxamide | LCMS (m/z): 548 [M + H]⁺. ¹H NMR (400 MHz, DMSO-d₆) δ 13.00 (br, 1H), 8.86 (s, 1H), 8.38 (s, 1H), 8.16 (s, 1H), 7.56 (s, 1H), 7.44 (s, 1H), 6.64-6.31 (m, 1H), 4.87-4.69 (m, 1H), 4.20-3.93 (m, 3H), 3.79-3.68 (m, 1H), 3.63 (s, 3H), 3.61-3.53 (m, 1H), 3.47-3.35 (m, 1H), 2.60 (s, 3H) |
| 386^{t} | 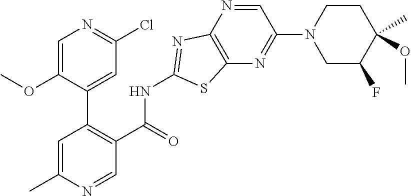 | 2′-chloro-N-(6-((3S or R,4R or S)-3-fluoro-4- methoxy-4- methylpiperidin-1- yl)thiazolo[4,5- b]pyrazin-2-yl)-5′- methoxy-6-methyl- [4,4′-bipyridine]-3- carboxamide | LCMS (m/z): 558 [M + H]⁺. ¹H NMR (300 MHz, DMSO-d₆) δ 13.01 (s, 1H), 8.84 (s, 1H), 8.41 (s, 1H), 8.17 (s, 1H), 7.59 (s, 1H), 7.47 (s, 1H), 4.71-4.48 (m, 1H), 4.00 (t, J = 13.9 Hz, 1H), 3.78- 3.66 (m, 2H), 3.62 (s, 3H), 3.53- 3.41 (m, 1H), 3.22 (s, 3H), 2.60 (s, 3H), 2.04-1.89 (m, 1H), 1.60- 1.47 (m, 1H), 1.27 (s, 3H) |
| 387^{t} | 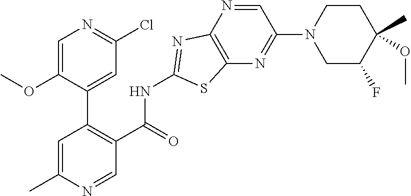 | 2′-chloro-N-(6-((3R or S,4S or R)-3-fluoro-4- methoxy-4- methylpiperidin-1- yl)thiazolo[4,5- b]pyrazin-2-yl)-5′- methoxy-6-methyl- [4,4′-bipyridine]-3- carboxamide | LCMS (m/z): 558 [M + H]⁺. ¹H NMR (400 MHz, DMSO-d₆) δ 13.00 (s, 1H), 8.84 (s, 1H), 8.41 (s, 1H), 8.17 (s, 1H), 7.58 (s, 1H), 7.46 (s, 1H), 4.68-4.48 (m, 1H), 3.51-3.41 (m, 1H), 3.73 (dd, J = 13.5, 6.5 Hz, 2H), 3.62 (s, 3H), 3.53-3.41 (m, 1H), 3.22 (s, 3H), 2.60 (s, 3H), 2.02-1.92 (m, 1H), 1.58-1.48 (m, 1H), 1.27 (s, 3H) |
| 388^{u} | 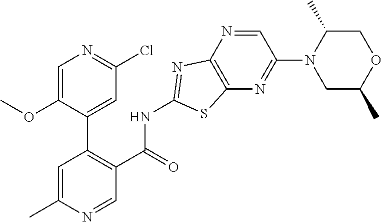 | 2′-chloro-N-(6- ((2S,5R)-2,5- dimethylmorpholino) thiazolo[4,5-b]pyrazin-2- yl)-5′- methoxy-6-methyl- [4,4′-bipyridine]-3- carboxamide | LCMS (m/z): 526 [M + H]⁺. ¹H NMR (400 MHz, DMSO-d₆) δ 12.98 (s, 1H), 8.86 (s, 1H), 8.26 (s, 1H), 8.16 (s, 1H), 7.56 (s, 1H), 7.43 (s, 1H), 4.36 (d, J = 7.7 Hz, 1H), 4.00 (d, J = 12.8 Hz, 1H), 3.78-3.68 (m, 2H), 3.62-3.58 (m, 4H), 2.80-2.70 (m, 1H), 2.60 (s, 3H), 1.24-1.00 (m, 6H) |
| 389^{u} | 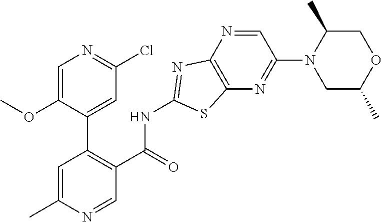 | 2′-chloro-N-(6- ((2R,5S)-2,5- dimethylmorpholino) thiazolo[4,5-b]pyrazin- 2-yl)-5′-methoxy-6- methyl-[4,4′- bipyridine]-3- carboxamide | LCMS (m/z): 526 [M + H]⁺. ¹H NMR (400 MHz, DMSO-d₆) δ 12.99 (s, 1H), 8.86 (s, 1H), 8.26 (s, 1H), 8.16 (s, 1H), 7.56 (s, 1H), 7.43 (s, 1H), 4.36 (d, J = 7.1 Hz, 1H), 4.04-3.95 (m, 1H), 3.73 (d, J = 5.8 Hz, 2H), 3.62 (d, J = 2.8 Hz, 3H), 3.56 (d, J = 9.7 Hz, 1H), 2.76 (t, J = 11.9 Hz, 1H), 2.59 (s, 3H), 1.18 (dd, J = 20.9, 6.4 Hz, 6H) |
| 390^{u} | 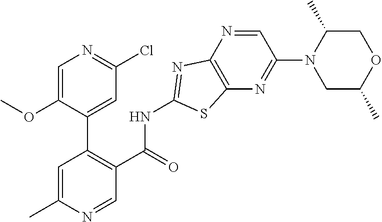 | 2′-chloro-N-(6- ((2R,5R)-2,5- dimethylmorpholino) thiazolo[4,5-b]pyrazin-2- yl)-5′-methoxy-6- methyl-[4,4′- bipyridine]-3- carboxamide | LCMS (m/z): 526 [M + H]⁺. ¹H NMR (400 MHz, DMSO-d₆) δ 8.90 (s, 1H), 8.22 (s, 1H), 8.15 (s, 1H), 7.51 (s, 1H), 7.38 (s, 1H), 4.06-3.94 (m, 3H), 3.62 (s, 3H), 3.46-3.38 (m, 3H), 2.58 (s, 3H), 1.20 (d, J = 6.4 Hz, 3H), 1.13 (d, J = 6.2 Hz, 3H) |
| 391^{u} | 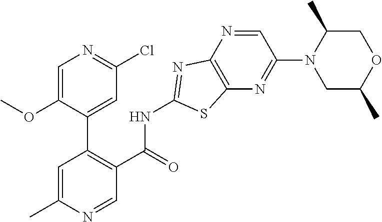 | 2′-chloro-N-(6- ((2S,5S)-2,5- dimethylmorpholino) thiazolo[4,5-b]pyrazin-2- yl)-5′-methoxy-6- methyl-[4,4′- bipyridine]-3- carboxamide | LCMS (m/z): 526 [M + H]⁺. ¹H NMR (400 MHz, DMSO-d₆) δ 8.90 (s, 1H), 8.22 (s, 1H), 8.15 (s, 1H), 7.51 (s, 1H), 7.38 (s, 1H), 4.06-3.94 (m, 3H), 3.62 (s, 3H), 3.46-3.38 (m, 3H), 2.58 (s, 3H), 1.20 (d, J = 6.4 Hz, 3H), 1.13 (d, J = 6.2 Hz, 3H) |

^{&}Single enantiomer, unknown stereochemistry
^{s}The chiral separation conditions for Example 376-379 are: Prep chiral HPLC (CHIRAL ART Cellulose-SB, 3 × 25 cm, 5 μm column; Mobile Phase A:
15% MeOH/85% Hexane:MTBE = 1:1 (0.5% 2M NH3—MeOH modifier), 40 mL/min. Peak 1 RT = 12.55 min (Example 376); Peak 2 RT = 14.92 min
(Examle 377); Peak 3 RT = 19.55 min (Example 378); Peak 4 RT = 23.76 min (Example 379)
^{t}Chiral separation conditions for Example 386 and 387: Chiral HPLC (CHIRALPAK IE, 3 × 25 cm, 5 μm column, 50%/50% Hexane with 0.1% formic acid
modifier/5:1 EtOH:MeCN, 40 mL/min flow rate) to afford Peak 1 RT = 28.84 min (Example 386) and Peak 2 RT = 35.7 min (Example 387)
^{u}Purification by prep-HPLC gave two peaks (mix of cis and mix of trans isomers): Xbridge Phenyl OBD Column, 19 × 150 mm, 5 um; 50-65% MeOH in
water 10 mmol/L NH4HCO3 and 0.05% NH₃H₂O modifier. Peak 1-mixture of Example 388 and 389; Peak 2-mixture of Example 390 and 391. Chiral
separation of peak 1 (mixture of Example 388 and 389): Prep-Chiral HPLC CHIRALPAK ID column, 2 × 25 cm, 5 μm; 15%/85% MeOH/MTBE:Hex =
1:1 with 0.1% formic acid modifier; 20 mL/min flow rate; wave length: 214/382 nm; two peaks:RT 14.27 min (Example 388), RT: 17.82 min (Example 389).
Chiral separation of peak 2 (mixture of Example 390 and 391): Prep-Chiral HPLC CHIRALPAK IA column, 2 × 25 cm, 5 μm; 10%/90% MeOH/MTBE:
Hex 1:1 with 0.1% formic acid modifier; 20 mL/min flow rate; wave length: 216/306 nm; two peaks: RT: 15.15 min (Example 390), RT: 22.13 min
(Example 391)

### Example 392A & 392B: Synthesis of (S or R)-2′-chloro-N-(6-(3,3-difluoro-4-methoxypiperidin-1-yl)thiazolo[4,5-b]pyrazin-2-yl)-5′-methoxy-6-methyl-[4,4′-bipyridine]-3-carboxamide (392A) & (R or S)-2′-chloro-N-(6-(3,3-difluoro-4-methoxypiperidin-1-yl)thiazolo[4,5-b]pyrazin-2-yl)-5′-methoxy-6-methyl-[4,4′-bipyridine]-3-carboxamide (392B) with arbitrarily assigned stereochemistry

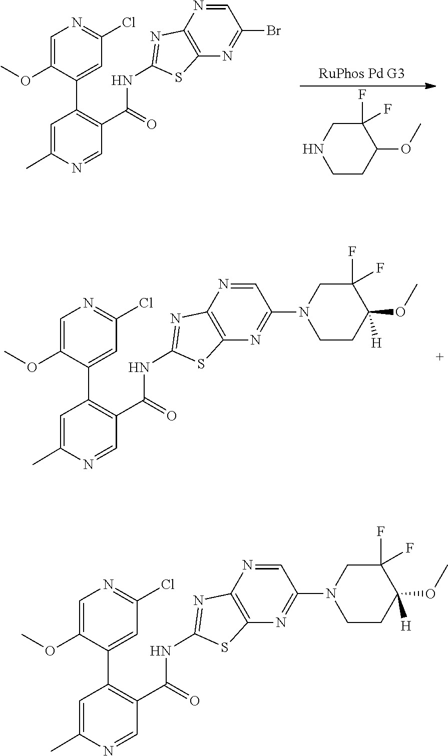

To a solution of 3,3-difluoro-4-methoxypiperidine (60 mg, 398 μmol), dicyclohexyl(2′,6′-diisopropoxy-[1,1′-biphenyl]-2-yl)phosphane (77 mg, 166 μmol) and N-(6-bromothiazolo[4,5-b]pyrazin-2-yl)-2′-chloro-5′-methoxy-6-methyl-[4,4′-bipyridine]-3-carboxamide (163 mg, 331 μmol) in DMF (2 mL) were added cesium carbonate (324 mg, 994 μmol) and RuPhos Pd G3 (69 mg, 83 μmol) in portions at 25° C. The mixture was stirred at 120° C. for 16 h under a nitrogen atmosphere. The resulting mixture was cooled to room temperature and concentrated under reduced pressure. The residue was purified directly by reverse-phase flash chromatography (0-100% MeCN in water with 0.1% formic acid modifier) to afford the crude product. The crude product was purified by prep-HPLC (35-53% MeCN in water with 0.1% formic acid modifier) to afford the racemic product. The racemic product purified by prep-chiral SFC (CHIRALPAK ID, 2×25 cm, 5 μm column, 70%/30% 1:1 MTBE/hexane (with 0.1% formic acid modifier)/MeOH at 20 min/mL flow rate) to afford two peaks:

Peak 1 RT=11.24: (S or R)-2′-chloro-N-(6-(3,3-difluoro-4-methoxypiperidin-1-yl)thiazolo[4,5-b]pyrazin-2-yl)-5′-methoxy-6-methyl-[4,4′-bipyridine]-3-carboxamide (4.4 mg, 2.3% yield) as a light yellow solid. LCMS (ESI, m/z): 562 [M+H]⁺. ¹H NMR (400 MHz, DMSO-d₆) δ 12.91 (s, 1H), δ 8.88 (s, 1H), 8.40 (s, 1H), 8.15 (s, 1H), 7.53 (s, 1H), 7.40 (s, 1H), 4.16-4.05 (m, 1H), 3.92-3.74 (m, 3H), 3.62 (s, 3H), 3.56 (s, 1H), 3.45 (s, 3H), 2.59 (s, 3H), 2.00 (d, J=13.1 Hz, 1H), 1.79 (s, 1H).

Peak 2 RT=14.89: (R or S)-2′-chloro-N-(6-(3,3-difluoro-4-methoxypiperidin-1-yl)thiazolo[4,5-b]pyrazin-2-yl)-5′-methoxy-6-methyl-[4,4′-bipyridine]-3-carboxamide (5.3 mg, 2.8% yield) as a light yellow solid. LCMS (ESI, m/z): 562 [M+H]⁺. ¹H NMR (400 MHz, DMSO-d₆) δ 12.96 (s, 1H), δ 8.88 (s, 1H), 8.41 (s, 1H), 8.15 (s, 1H), 7.54 (s, 1H), 7.41 (s, 1H), 4.18-4.05 (m, 1H), 3.90-3.74 (m, 3H), 3.62 (s, 3H), 3.56 (s, 1H), 3.45 (s, 3H), 2.59 (s, 3H), 2.00 (s, 1H), 1.79 (d, J=10.7 Hz, 1H).

TABLE AY
The following examples were prepared using a similar procedure to that described
for Example 392A

| Ex. | | | |
|---|---|---|---|
| No. | Structure | Compound Name | Characterization Data |
| 393 | 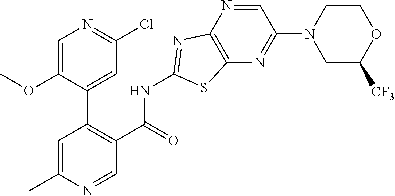 [SMILES: COc1cnc(Cl)cc1-c1cc(C)ncc1C(=O)Nc1nc2ncc(N3CCO[C@@H](C(F)(F)F)C3)nc2s1] | (R)-2′-chloro-5′- methoxy-6-methyl-N- (6-(2- (trifluoromethyl)morpholino) thiazolo[4,5-b]pyrazin-2-yl)- [4,4′-bipyridine]-3-carboxamide | LCMS (m/z): 566 [M + H]⁺. ¹H NMR (300 MHz, DMSO-d₆) δ 13.09 (s, 1H), 8.89 (s, 1H), 8.41 (s, 1H), 8.16 (s, 1H), 7.55 (s, 1H), 7.43 (s, 1H), 4.37 (d, J = 11.2 Hz, 2H), 4.13 (t, J = 10.8 Hz, 2H), 3.83-3.70 (m, 1H), 3.62 (s, 3H), 3.06 (dd, J = 24.0, 12.4 Hz, 2H), 2.60 (s, 3H) |

### Example 394: Synthesis of 2′-chloro-N-(6-((3R,5R)-3,5-dimethylmorpholino)thiazolo[4,5-b]pyrazin-2-yl)-5′-methoxy-6-methyl-[4,4′-bipyridine]-3-carboxamide

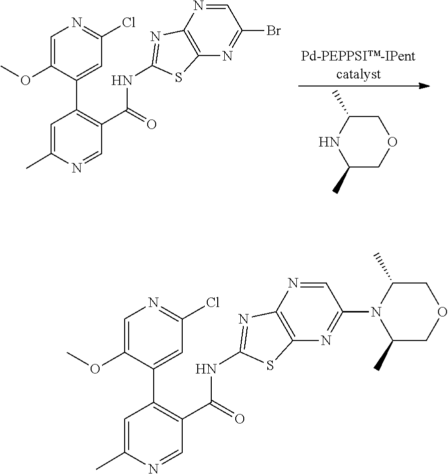

A mixture of N-(6-bromothiazolo[4,5-b]pyrazin-2-yl)-2′-chloro-5′-methoxy-6-methyl-[4,4′-bipyridine]-3-carboxamide (200 mg, 0.41 mmol) and sodium 2-methylpropan-2-olate (117 mg, 1.22 mmol), (3R,5R)-3,5-dimethylmorpholine HCl salt (93 mg, 0.61 mmol), and Pd-PEPPSI™-IPent catalyst (32 mg, 0.04 mmol) in 1,4-dioxane (20 mL) was stirred for 16 h at 100° C. under a N₂ atmosphere. The resulting mixture was cooled to room temperature and concentrated under reduced pressure. The crude product was purified by Prep-HPLC with the following conditions (Column: XSelect CSH Fluoro Phenyl, 30*150 mm, 5 m; Mobile Phase A: Water(0.1% HCOOH), Mobile Phase B: ACN; Flow rate: 60 mL/min mL/min; Gradient: 33% B to 53% B in 10 min; Wave Length: 254 nm/220 nm; RT1(min): 8.31) to afford 2′-chloro-N-(6-((3R,5R)-3,5-dimethylmorpholino)thiazolo[4,5-b]pyrazin-2-yl)-5′-methoxy-6-methyl-[4,4′-bipyridine]-3-carboxamide (6 mg, 3% yield) as a yellow solid. LCMS (ESI, m/z): 526 [M+H]⁺. ¹H NMR (300 MHz, DMSO-d₆) δ 13.12 (s, 1H), 8.87 (s, 1H), 8.38 (s, 1H), 8.17 (s, 1H), 7.58 (s, 1H), 7.46 (s, 1H), 3.92 (dd, J=11.6, 3.3 Hz, 4H), 3.64 (s, 3H), 3.56-3.45 (m, 2H), 2.61 (s, 3H), 1.05 (d, J=6.2 Hz, 6H).

TABLE AZ
The following examples were prepared using a similar procedure to that described
for Example 394

| Ex. | | | |
|---|---|---|---|
| No. | Structure | Compound Name | Characterization Data |
| 395 | 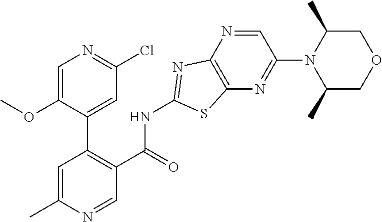 [SMILES: COc1cnc(Cl)cc1-c1cc(C)ncc1C(=O)Nc1nc2ncc(N3[C@H](C)COC[C@@H]3C)nc2s1] | 2′-chloro-N-(6-((3R,5S)- 3,5-dimethylmorpholino) thiazolo[4,5-b]pyrazin-2-yl)- 5′-methoxy-6-methyl- [4,4′-bipyridine]-3-carboxamide | LCMS (m/z): 526 [M + H]⁺. ¹H NMR (300 MHz, DMSO-d₆) δ 12.99 (s, 1H), 8.85 (s, 1H), 8.18 (d, J = 5.1 Hz, 2H), 7.58 (s, 1H), 7.45 (s, 1H), 4.26 (s, 2H), 3.79 (d, J = 11.4 Hz, 2H), 3.63 (s, 5H), 2.60 (s, 3H), 1.26 (d, J = 6.8 Hz, 6H) |
| 396 | 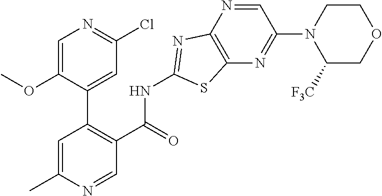 [SMILES: COc1cnc(Cl)cc1-c1cc(C)ncc1C(=O)Nc1nc2ncc(N3CCOC[C@@H]3C(F)(F)F)nc2s1] | (R)-2′-chloro-5′- methoxy-6-methyl-N-(6-(3- (trifluoromethyl)morpholino) thiazolo[4,5-b]pyrazin-2-yl)-[4,4′- bipyridine]-3-carboxamide | LCMS (m/z): 566 [M + H]⁺. ¹H NMR (400 MHz, DMSO-d₆) δ 13.10 (s, 1H), 8.87 (s, 1H), 8.40 (s, 1H), 8.16 (s, 1H), 7.56 (s, 1H), 7.44 (s, 1H), 5.33-5.26 (m, 1H), 4.21 (d, J = 12.8 Hz, 1H), 4.10-3.96 (m, 2H), 3.80 (d, J = 12.8 Hz, 1H), 3.63 (s, 3H), 3.61-3.55 (m, 1H), 3.41- 3.36 (m, 1H), 2.60 (s, 3H) |
| 397 | 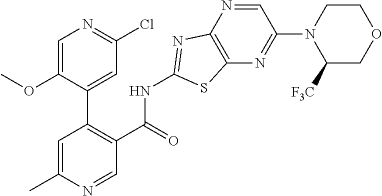 [SMILES: COc1cnc(Cl)cc1-c1cc(C)ncc1C(=O)Nc1nc2ncc(N3CCOC[C@H]3C(F)(F)F)nc2s1] | (S)-2′-chloro-5′- methoxy-6-methyl-N-(6-(3- (trifluoromethyl)morpholino) thiazolo[4,5-b]pyrazin-2-yl)-[4,4′- bipyridine]-3-carboxamide | LCMS (m/z): 566 [M + H]⁺. ¹H NMR (400 MHz, DMSO-d₆) δ 13.10 (s, 1H), 8.87 (s, 1H), 8.41 (s, 1H), 8.16 (s, 1H), 7.57 (s, 1H), 7.44 (s, 1H), 5.35-5.24 (m, 1H), 4.21 (d, J = 12.8 Hz, 1H), 4.03 (dd, J = 22.1, 11.5 Hz, 2H), 3.80 (d, J = 12.7 Hz, 1H), 3.62 (s, 3H), 3.61-3.55 (m, 1H), 2.60 (s, 3H), 2.54 (s, 1H) |

### Example 398: Synthesis of 2′-chloro-5′-methoxy-6-methyl-N-(6-(pyrrolidin-1-yl)thiazolo[4,5-b]pyrazin-2-yl)-[4,4′-bipyridine]-3-carboxamide

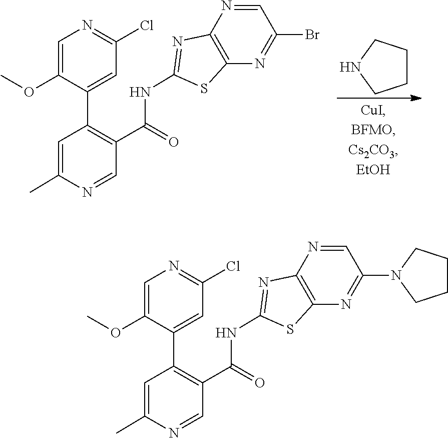 [SMILES: COc1cnc(Cl)cc1-c1cc(C)ncc1C(=O)Nc1nc2ncc(Br)nc2s1 ; C1CCNC1 ; COc1cnc(Cl)cc1-c1cc(C)ncc1C(=O)Nc1nc2ncc(N3CCCC3)nc2s1]

To a solution of N-(6-bromothiazolo[4,5-b]pyrazin-2-yl)-2′-chloro-5′-methoxy-6-methyl-[4,4′-bipyridine]-3-carboxamide (100 mg, 0.2 mmol) and pyrrolidine (22 mg, 0.3 mmol) in EtOH (3 mL) were added CuI (4 mg, 0.02 mmol), BFMO (5 mg, 0.02 mmol), and Cs₂CO₃ (199 mg, 0.6 mmol) at 25° C. The reaction mixture was charged with N₂ three times at 25° C., and stirred at 100° C. for 16 h under a nitrogen atmosphere. The resulting mixture was cooled to 25° C. and concentrated under reduced pressure. The residue was purified by prep-TLC (DCM/MeOH) (10/1) to afford crude product. The crude product (60 mg) was purified by prep-HPLC (27-45% MeCN in water with 0.1% formic acid modifier) to afford 2′-chloro-5′-methoxy-6-methyl-N-(6-(pyrrolidin-1-yl)thiazolo[4,5-b]pyrazin-2-yl)-[4,4′-bipyridine]-3-carboxamide (7.8 mg, 7.9% yield) as a light yellow solid. LCMS (ESI, m/z): 482 [M+H]⁺. ¹H NMR (400 MHz, DMSO-d₆) δ 12.90 (s, 1H), 8.84 (s, 1H), 8.17 (s, 1H), 7.96 (s, 1H), 7.56 (s, 1H), 7.45 (s, 1H), 3.63 (s, 3H), 3.51-3.44 (m, 4H), 2.60 (s, 3H), 2.02-1.94 (m, 4H).

TABLE BA
The following examples were prepared using a similar procedure to that described
for Example 398

| Ex. | | | |
|---|---|---|---|
| No. | Structure | Compound Name | Characterization Data |
| 399 | 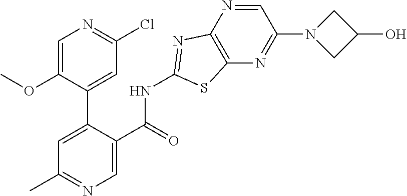 [SMILES: COc1cnc(Cl)cc1-c1cc(C)ncc1C(=O)Nc1nc2ncc(N3CC(O)C3)nc2s1] | 2′-chloro-N-(6-(3- hydroxyazetidin-1- yl)thiazolo[4,5- b]pyrazin-2-yl)-5′- methoxy-6-methyl- [4,4′-bipyridine]-3- carboxamide | LCMS (m/z): 484 [M + H]⁺. ¹H NMR (400 MHz, DMSO-d₆) δ 12.98 (s, 1H), 8.83 (s, 1H), 8.17 (s, 1H), 7.87 (s, 1H), 7.57 (s, 1H), 7.46 (s, 1H), 5.76 (d, J = 6.4 Hz, 1H), 4.63 (d, J = 5.5 Hz, 1H), 4.30 (d, J = 6.5 Hz, 2H), 3.83 (d, J = 9.0 Hz, 2H), 3.62 (s, 3H), 2.60 (s, 3H) |

### Example 400: Synthesis of 2′-chloro-N-(6-(4-(difluoromethyl)-4-hydroxycyclohex-1-en-1-yl)thiazolo[4,5-b]pyrazin-2-yl)-5′-methoxy-6-methyl-[4,4′-bipyridine]-3-carboxamide

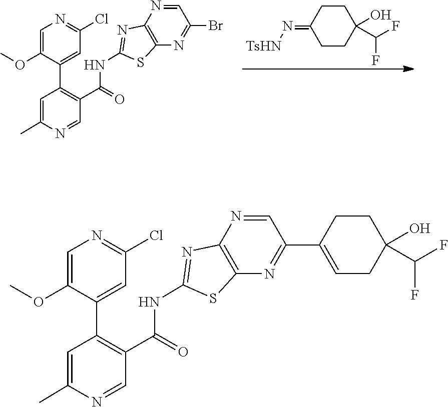

To a stirred solution of N′-(4-(difluoromethyl)-4-hydroxycyclohexylidene)-4-methylbenzenesulfonohydrazide (300 mg, 0.903 mmol) and N-(6-bromothiazolo[4,5-b]pyrazin-2-yl)-2′-chloro-5′-methoxy-6-methyl-[4,4′-bipyridine]-3-carboxamide (444 mg, 0.903 mmol) in 1,4-dioxane/H₂O (10 mL) were added Pd₂(dba)₃ (82 mg, 0.09 mmol), X-Phos (86 mg, 0.18 mmol) and lithium tert-butoxide (159 mg, 1.99 mmol) in portions at 25° C. under a nitrogen atmosphere. The resulting mixture was stirred for 16 h at 100° C. under a nitrogen atmosphere. The mixture was cooled to 25° C., and extracted with EtOAc (5×100 ml). After filtration, the filtrate was concentrated under reduced pressure. The residue was purified by Prep-HPLC (33-53% MeCN in water with 0.1% formic acid modifier) to afford 2′-chloro-N-(6-(4-(difluoromethyl)-4-hydroxycyclohex-1-en-1-yl)thiazolo[4,5-b]pyrazin-2-yl)-5′-methoxy-6-methyl-[4,4′-bipyridine]-3-carboxamide (48 mg, 9.4% yield) as white solid. LCMS (ESI, m/z): 559 [M+H]⁺. ¹H NMR (400 MHz, DMSO-d₆) δ 13.39 (s, 1H), 8.90 (s, 1H), 8.83 (s, 1H), 8.20-8.12 (m, 1H), 7.59 (s, 1H), 7.46 (s, 1H), 6.74 (s, 1H), 5.84 (s, 1H), 5.24 (d, J=1.8 Hz, 2H), 3.63 (d, J=1.5 Hz, 3H), 2.71 (d, J=16.9 Hz, 3H), 2.63-2.59 (m, 1H), 2.43 (s, 1H), 2.29 (s, 1H), 1.86 (d, J=12.8 Hz, 1H), 1.63 (td, J=12.4, 6.0 Hz, 1H).

TABLE BB
The following examples were prepared using a similar procedure to that described
for Example 400

| Ex. | | | |
|---|---|---|---|
| No. | Structure | Compound Name | Characterization Data |
| 401 | 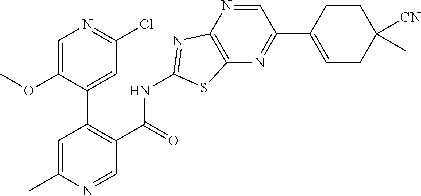 [SMILES: COc1cnc(Cl)cc1-c1cc(C)ncc1C(=O)Nc1nc2ncc(C3=CCC(C)(C#N)CC3)nc2s1] | 2′-chloro-N-(6-(4- cyano-4-methyl- cyclohex-1- en-1-yl) thiazolo[4,5-b] pyrazin-2-yl)-5′- methoxy-6-methyl- [4,4′- bipyridine]-3- carboxamide | LCMS (m/z): 532 [M + H]⁺. ¹H NMR (300 MHz, DMSO-d₆) δ 13.44 (s, 1H); 8.89 (d, J = 2.8 Hz, 2H), 8.17 (s, 1H), 7.61 (s, 1H), 7.48 (s, 1H), 6.83 (s, 1H), 3.63 (s, 3H), 2.87- 2.64 (m, 3H), 2.61 (s, 3H), 2.40 (d, J = 18.4 Hz, 1H), 2.14 (d, J = 12.9 Hz, 1H), 1.82-1.68 (m, 1H), 1.44 (s, 3H) |
| 402 | 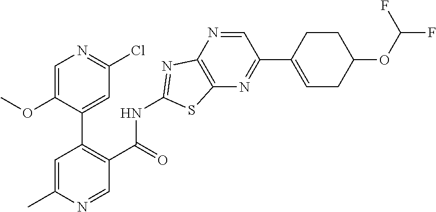 [SMILES: COc1cnc(Cl)cc1-c1cc(C)ncc1C(=O)Nc1nc2ncc(C3=CCC(OC(F)F)CC3)nc2s1] | 2′-chloro-N-(6- (4-(difluoro- methoxy)cyclohex- 1-en-1-yl) thiazolo[4,5-b] pyrazin-2-yl)-5′- methoxy-6-methyl- [4,4′-bipyridine]- 3-carboxamide | LCMS (m/z): 559 [M + H]⁺. ¹H NMR (400 MHz, DMSO-d₆) δ 13.42 (br, 1H), 8.90-8.83 (m, 2H), 8.17 (s, 1H), 7.59 (s, 1H), 7.47 (s, 1H), 6.99- 6.53 (m, 2H), 4.53-4.43 (m, 1H), 3.63 (s, 3H), 2.74-2.57 (m, 6H), 2.46-2.31 (m, 1H), 2.09-1.98 (m, 1H), 1.95-1.85 (m, 1H) |
| 403 | 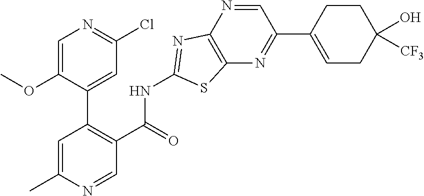 [SMILES: COc1cnc(Cl)cc1-c1cc(C)ncc1C(=O)Nc1nc2ncc(C3=CCC(O)(C(F)(F)F)CC3)nc2s1] | 2′-chloro-N-(6- (4-hydroxy-4- (trifluoromethyl) cyclohex-1-en-1- yl)thiazolo[4,5- b]pyrazin-2-yl)-5′- methoxy-6-methyl- [4,4′-bipyridine]- 3-carboxamide | LCMS (m/z): 577 [M + H]⁺. ¹H NMR (300 MHz, DMSO-d₆) δ 13.40 (s, 1H), 8.93 (s, 1H), 8.77 (s, 1H), 8.15 (s, 1H), 7.54 (s, 1H), 7.40 (s, 1H), 6.69 (s, 1H), 5.93 (s, 1H), 3.62 (s, 3H), 2.77 (d, J = 17.9 Hz, 1H), 2.59 (s, 5H), 2.39 (d, J = 17.1 Hz, 1H), 2.00 (d, J = 8.9 Hz, 1H), 1.74 (t, J = 6.4 Hz, 1H) |
| 404 | 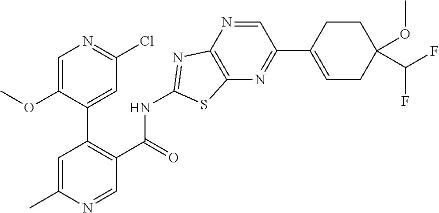 [SMILES: COc1cnc(Cl)cc1-c1cc(C)ncc1C(=O)Nc1nc2ncc(C3=CCC(OC)(C(F)F)CC3)nc2s1] | 2′-chloro-N-(6-(4- (difluoromethyl)-4- methoxycyclohex- 1-en-1-yl) thiazolo[4,5-b] pyrazin-2-yl)-5′- methoxy-6-methyl- [4,4′-bipyridine]-3- carboxamide | LCMS (m/z): 573 [M + H]⁺. ¹H NMR (400 MHz, DMSO-d₆) δ 8.87 (d, J = 10.8 Hz, 2H), 8.18 (s, 1H), 7.60 (s, 1H), 7.48 (s, 1H), 6.77 (s, 1H), 6.14 (t, J = 55.1 Hz, 1H), 3.63 (s, 3H), 3.31 (s, 3H), 2.72 (d, J = 17.8 Hz, 2H), 2.61 (s, 3H), 2.42 (d, J = 19.3 Hz, 2H), 2.11 (d, J = 13.1 Hz, 1H), 1.77-1.65 (m, 1H) |
| 405 | 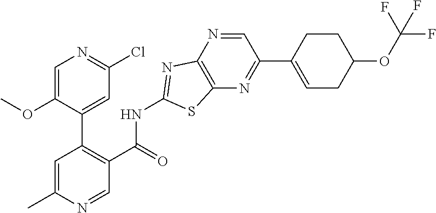 [SMILES: COc1cnc(Cl)cc1-c1cc(C)ncc1C(=O)Nc1nc2ncc(C3=CCC(OC(F)(F)F)CC3)nc2s1] | 2′-chloro-5′- methoxy-6-methyl- N-(6-(4- (trifluoromethoxy) cyclohex-1-en-1- yl)thiazolo[4,5- b]pyrazin-2-yl)- [4,4′-bipyridine]-3- carboxamide | LCMS (m/z): 577 [M + H]⁺. ¹H NMR (400 MHz, DMSO-d₆) δ 13.43 (s, 1H), 8.88 (d, J = 5.7 Hz, 2H), 8.18 (s, 1H), 7.61 (s, 1H), 7.48 (s, 1H), 6.74 (s, 1H), 4.82 (s, 1H), 3.63 (s, 3H), 2.74 (d, J = 21.9 Hz, 3H), 2.61 (s, 3H), 2.48-2.43 (m, 1H), 2.12- 1.98 (m, 2H) |

### Example 406: Synthesis of 2′-chloro-N-(6-(4-(difluoromethyl)-4-methoxycyclohexyl)thiazolo[4,5-b]pyrazin-2-yl)-5′-methoxy-6-methyl-1[4,4′-bipyridine]-3-carboxamide

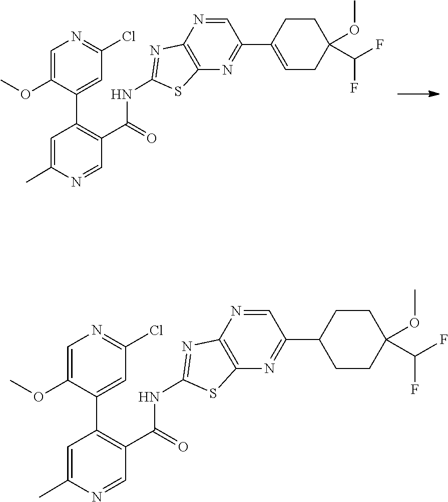 [SMILES: COc1cnc(Cl)cc1-c1cc(C)ncc1C(=O)Nc1nc2ncc(C3=CCC(OC)(C(F)F)CC3)nc2s1 ; COc1cnc(Cl)cc1-c1cc(C)ncc1C(=O)Nc1nc2ncc(C3CCC(OC)(C(F)F)CC3)nc2s1]

To a stirred solution of 2′-chloro-N-(6-(4-(difluoromethyl)-4-methoxycyclohex-1-en-1-yl) thiazolo[4,5-b]pyrazin-2-yl)-5′-methoxy-6-methyl-[4,4′-bipyridine]-3-carboxamide (80 mg, 0.14 mmol) in MeOH (5 mL) was added PtO₂ (50 mg, 0.22 mmol) in portions at room temperature under a hydrogen atmosphere. The resulting mixture was stirred for 2 days at room temperature under a hydrogen atmosphere. The mixture was purified by prep-HPLC with the following conditions (55-75% o MeOH in water with 0.100 formic acid modifier) to afford 2′-chloro-N-(6-(4-(difluoromethyl)-4-methoxycyclohexyl)thiazolo[4,5-b]pyrazin-2-yl)-5 methoxy-6-methyl-[4,4′-bipyridine]-3-carboxamide (6.7 mg, 8% yield) as a yellow solid. LCMS (ESI, m/z): 575 [M+H]⁺. ¹HNMR (400 MHz, DMSO-d₆) δ13.38 (s, 1H), 8.88 (s, 1H), 8.56 (s, 1H), 8.17 (s, 1H), 7.60 (s, 1H), 7.47 (s, 1H), 6.10 (t, J=65.7, 54.9 Hz, 1H), 3.62 (s, 3H), 3.32 (d, J=5.7 Hz, 3H), 3.10-2.90 (i, 1H), 2.61 (s, 3H), 1.99-1.91 (2, 3H), 1.80 (s, 3H), 1.72-1.64 (m, 1H), 1.55 (m, 1H).

TABLE BC
The following examples were prepared using a similar procedure to that described
for Example 406

| Ex. | | Compound | |
|---|---|---|---|
| No. | Structure | Name | NMR |
| 407 | 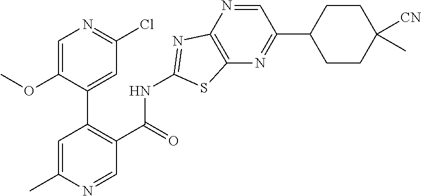 [SMILES: COc1cnc(Cl)cc1-c1cc(C)ncc1C(=O)Nc1nc2ncc(C3CCC(C)(C#N)CC3)nc2s1] | 2′-chloro-N- (6-(4-cyano- 4-methyl- cyclohexyl) thiazolo[4,5- b]pyrazin- 2-yl)-5′- methoxy-6- methyl-[4,4′- bipyridine]- 3- carboxamide | LCMS (m/z): 534 [M + H]⁺. ¹H NMR (400 MHz, DMSO-d₆) δ 13.39 (s, 1H), 8.89 (s, 1H), 8.59 (d, J = 20.3 Hz, 1H), 8.16 (s, 1H), 7.60 (s, 1H), 7.48 (s, 1H), 3.62 (s, 3H), 2.92 (tt, J = 12.1, 3.7 Hz, 1H), 2.61 (s, 3H), 2.08- 1.76 (m, 6H), 1.55 (td, J = 13.4, 3.5 Hz, 2H), 1.39 (d, J = 6.1 Hz, 3H) |
| 408^{%,v} | 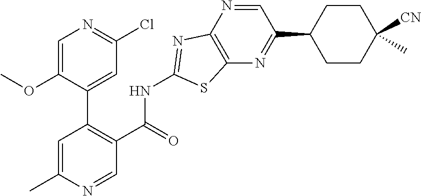 [SMILES: COc1cnc(Cl)cc1-c1cc(C)ncc1C(=O)Nc1nc2ncc([C@H]3CC[C@@](C)(C#N)CC3)nc2s1] | (cis or trans)- 2′-chloro-N- (6-(4-cyano- 4-methyl- cyclohexyl) thiazolo[4,5- b]pyrazin-2- yl)-5′- methoxy-6- methyl-[4,4′- bipyridine]-3- carboxamide | LCMS (m/z): 534 [M + H]⁺. ¹H NMR (400 MHz, DMSO-d₆) δ 13.33 (s, 1H), 8.91 (s, 1H), 8.57 (s, 1H), 8.16 (s, 1H), 7.56 (s, 1H), 7.43 (s, 1H), 3.62 (s, 3H), 3.06-3.00 (m, 1H), 2.60 (s, 3H), 2.00-1.78 (m, 8H), 1.40 (s, 3H) |
| 409^{%,v} | 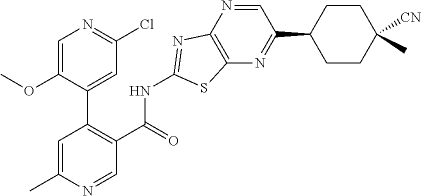 [SMILES: COc1cnc(Cl)cc1-c1cc(C)ncc1C(=O)Nc1nc2ncc([C@H]3CC[C@](C)(C#N)CC3)nc2s1] | (trans or cis)- 2′-choro-N- (6-(4-cyano- 4-methyl- cyclohexyl) thiazolo[4,5- b]pyrazin-2- yl)-5′- methoxy-6- methyl-[4,4′- bipyridine]-3- carboxamide | LCMS (m/z): 534 [M + H]⁺. ¹H NMR (400 MHz, DMSO-d₆) δ 13.38 (s, 1H), 8.91 (s, 1H), 8.52 (s, 1H), 8.16 (s, 1H), 7.58 (s, 1H), 7.45 (s, 1H), 3.62 (s, 3H), 2.94-2.87 (m, 1H), 2.61 (s, 3H), 2.08-1.90 (m, 4H), 1.88- 1.87 (m, 2H), 1.55 (td, J = 13.5, 3.5 Hz, 2H), 1.38 (s, 3H) |
| 410^{%,w} | 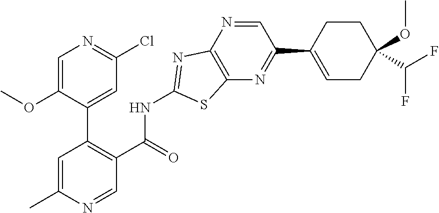 | (cis or trans)- 2′-chloro-N- (6-(4-(difluoro- methyl)-4- methoxycyclo- hexyl)thiazolo [4,5-b]pyrazin- 2-yl)-5′- methoxy-6- methyl-[4,4′- bipyridine]-3- carboxamide | LCMS (m/z): 575 [M + H]⁺. ¹H NMR (400 MHz, DMSO-d6) δ 13.37 (s, 1H), 8.88 (s, 1H), 8.57 (s, 1H), 8.17 (s, 1H), 7.60 (s, 1H), 7.48 (s, 1H), 5.95 (d, J = 55.2 Hz, 1H), 3.62 (s, 3H), 3.33 (s, 3H), 2.99-2.89 (m, 1H), 2.61 (s, 3H), 1.96 (d, J = 13.3 Hz, 2H), 1.85-1.75 (m, 4H), 1.55 (dt, J = 13.6, 8.9 Hz, 2H) |
| 411^{%,w} | 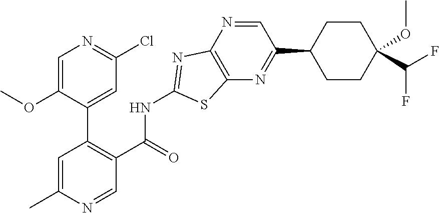 [SMILES: COc1cnc(Cl)cc1-c1cc(C)ncc1C(=O)Nc1nc2ncc([C@H]3CC[C@@](OC)(C(F)F)CC3)nc2s1] | (trans or cis)- 2′-chloro-N- (6-(4-(difluoro- methyl)-4- methoxycyclo- hexyl)thiazolo [4,5-b]pyrazin- 2-yl)-5′- methoxy-6- methyl-[4,4′- bipyridine]-3- carboxamide | LCMS (m/z): 575 [M + H]⁺. ¹H NMR (400 MHz, DMSO-d6) δ 8.92 (s, 1H), 8.57 (s, 1H), 8.15 (s, 1H), 7.55 (s, 1H), 7.42 (s, 1H), 6.18 (t, J = 54.5 Hz, 1H), 3.62 (s, 3H), 3.31 (s, 3H), 3.09 (t, J = 6.4 Hz, 1H), 2.60 (s, 3H), 2.03-1.81 (m, 6H), 1.71-1.63 (m, 2H) |
| 412^{%,x} | 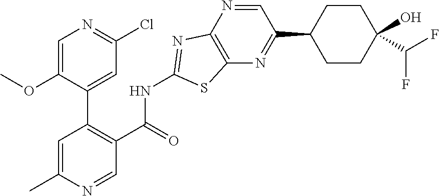 [SMILES: COc1cnc(Cl)cc1-c1cc(C)ncc1C(=O)Nc1nc2ncc([C@H]3CC[C@](O)(C(F)F)CC3)nc2s1] | (cis or trans)- 2′-chloro-N- (6-(4-(difluoro- methyl)-4- hydroxycyclo- hexyl)thiazolo [4,5-b]pyrazin- 2-yl)-5′- methoxy-6- methyl-[4,4′- bipyridine]-3- carboxamide | LCMS (m/z): 561 [M + H]⁺. ¹H NMR (400 MHz, DMSO-d6) δ 13.37 (s, 1H) 8.94 (s, 1H), 8.51 (s, 1H), 8.14 (s, 1H), 7.51 (d, J = 3.8 Hz, 1H), 7.37 (d, J = 4.4 Hz, 1H), 5.95 (m, 1H), 5.10 (s, 1H), 3.62 (s, 3H), 3.03 (s, 1H), 2.59 (s, 3H), 1.96-1.85 (m, 6H), 1.51 (s, 2H) |
| 413^{%,x} | 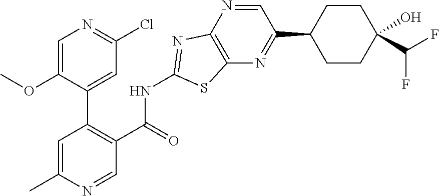 [SMILES: COc1cnc(Cl)cc1-c1cc(C)ncc1C(=O)Nc1nc2ncc([C@H]3CC[C@@](O)(C(F)F)CC3)nc2s1] | (trans or cis) 2′-chloro-N- (6-(4-(difluoro- methyl)-4- hydroxycyclo- hexyl)thiazolo [4,5-b]pyrazin- 2-yl)-5′- methoxy-6- methyl-[4,4′- bipyridine]-3- carboxamide | LCMS (m/z): 561 [M + H]⁺. ¹H NMR (400 MHz, DMSO-d6) δ 13.34 (s, 1H), 8.90 (s, 1H), 8.51 (s, 1H), 8.16 (s, 1H), 7.57 (s, 1H), 7.44 (s, 1H), 5.71 (m, 1H), 5.09 (s, 1H), 3.62 (s, 3H), 2.86 (t, J = 12.1 Hz, 1H), 2.60 (s, 3H), 1.97 (q, J = 11.6, 11.1 Hz, 2H), 1.73 (t, J = 12.3 Hz, 4H), 1.54 (dd, J = 14.7, 10.6 Hz, 2H) |
| 414^{%,y} | 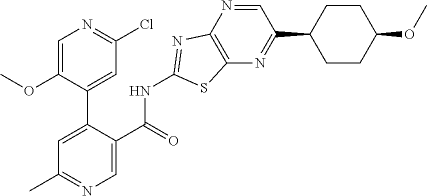 | (cis or trans)- 4-(2-methoxy- phenyl)-6- methyl-N-(6- (tetrahydro- furan-3-yl) imidazo[2,1- b][1,3,4]thia- diazol-2-yl) nicotinamide | LCMS (m/z): 525 [M + H]⁺. ¹H NMR (400 MHz, DMSO-d₆) δ 13.29 (s, 1H), 8.87 (s, 1H), 8.38 (s, 1H), 8.08 (s, 1H), 7.46 (s, 1H), 7.32 (s, 1H), 3.55 (s, 3H), 3.20 (s, 3H), 2.75 (s, 1H), 2.52 (s, 1H), 2.05 (d, J = 12.3 Hz, 3H), 1.86 (d, J = 13.1 Hz, 2H), 1.60 (d, J = 12.8 Hz, 2H), 1.54 (d, J = 12.5 Hz, 2H), 1.27-1.16 (m, 2H) |
| 415^{%,y} | 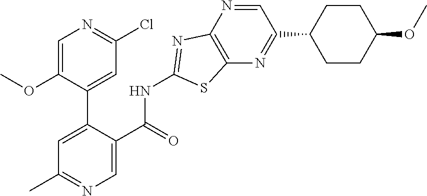 | (trans or cis)- 4-(2-methoxy- phenyl)-6- methyl-N-(6- (tetrahydro- furan-3-yl) imidazo[2,1-b] [1,3,4] thiadiazol-2- yl)nicotinamide | LCMS (m/z): 525 [M + H]⁺. ¹H NMR (400 MHz, DMSO-d₆) δ 13.35 (s, 1H), 8.89 (s, 1H), 8.52 (s, 1H), 8.16 (s, 1H), 7.59 (s, 1H), 7.46 (s, 1H), 3.62 (s, 3H), 3.52-3.46 (m, 1H), 3.25 (s, 3H), 2.97-2.87 (m, 1H), 2.61 (s, 3H), 2.01-1.90 (m, 2H), 1.85 (d, J = 12.4, 3.4 Hz, 2H), 1.68 (s, 2H), 1.49 (s, 2H) |
| 416* | 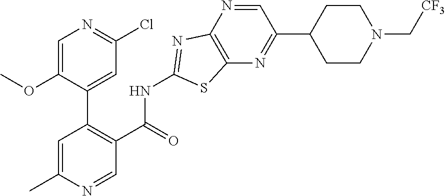 [SMILES: COc1cnc(Cl)cc1-c1cc(C)ncc1C(=O)Nc1nc2ncc(C3CCN(CC(F)(F)F)CC3)nc2s1] | 2′-chloro-5′- methoxy-6- methyl-N-(6- (1-(2,2,2- trifluoroethyl) piperidin-4-yl) thiazolo[4,5-b] pyrazin-2-yl)- [4,4′- bipyridine]-3- carboxamide | LCMS (m/z): 578 [M + H]⁺. ¹H NMR (400 MHz, DMSO-d₆) δ 13.37 (s, 1H), 8.89 (d, J = 1.8 Hz, 1H), 8.58 (d, J = 1.8 Hz, 1H), 8.17 (d, J = 1.8 Hz, 1H), 7.60 (d, J = 1.8 Hz, 1H), 7.47 (d, J = 1.8 Hz, 1H), 3.62 (d, J = 1.8 Hz, 3H), 3.27-3.16 (m, 2H), 3.04 (d, J = 11.1 Hz, 2H), 2.94-2.80 (m, 1H), 2.60 (s, 3H), 2.54 (d, J = 1.8 Hz, 1H), 2.49-2.42 (m, 1H), 1.85 (t, J = 5.3 Hz, 4H) |
| 417^{%,z} | 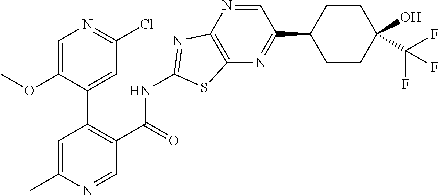 [SMILES: COc1cnc(Cl)cc1-c1cc(C)ncc1C(=O)Nc1nc2ncc([C@H]3CC[C@](O)(C(F)(F)F)CC3)nc2s1] | (cis or trans)- 2′-chloro-N- (6-(4-hydroxy- 4-(trifluoro- methyl)cyclo- hexyl)thiazolo [4,5-b]pyrazin- 2-yl)-5′- methoxy-6- methyl-[4,4′- bipyridine]-3- carboxamide | LCMS (m/z): 579 [M + H]⁺. ¹H NMR (400 MHz, methanol-d₄): δ 8.90 (s, 1H), 8.47 (s, 1H), 8.05 (s, 1H), 7.49 (s, 1H), 7.40 (s, 1H), 3.71 (s, 3H), 3.23-3.19 (m, 1H), 2.67 (s, 3H), 2.15-2.06 (m, 6H), 1.73-1.68 (m, 2H), 1.28 (s, 1H), 0.90-0.87 (m, 1H) |
| 418^{%,z} | 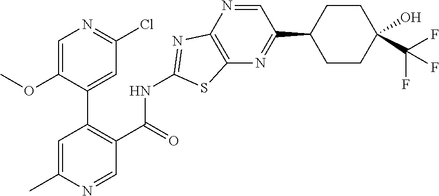 [SMILES: COc1cnc(Cl)cc1-c1cc(C)ncc1C(=O)Nc1nc2ncc([C@H]3CC[C@@](O)(C(F)(F)F)CC3)nc2s1] | (trans or cis)- 2′-chloro-N-(6- (4-hydroxy-4- (trifluoro- methyl)cyclo- hexyl)thiazolo [4,5-b]pyrazin- 2-yl)-5′- methoxy-6- methyl-[4,4′- bipyridine]-3- carboxamide | LCMS (m/z): 579 [M + H]⁺. ¹H NMR (400 MHz, methanol-d₄): δ 8.87 (s, 1H), 8.46 (s, 1H), 8.07 (s, 1H), 7.51 (s, 1H), 7.44 (s, 1H), 3.71 (s, 3H), 2.94 (d, J = 3.4 Hz, 1H), 2.68 (s, 3H), 2.10 (dd, J = 12.9, 3.5 Hz, 2H), 1.99 (d, J = 13.2 Hz, 2H), 1.89-1.76 (m, 4H) |

^{%}Unknown stereochemistry
*The precursor to Example 416 was made via a Suzuki reaction as described in Example 268.
^{v}Chiral separation conditions for Examples 408 and 409: Prep-Chiral HPLC CHIRALPAK ID column 3 × 25 cm, 5 μm; 30%/70% MeOH/Hexane:MTBE
with 0.5% formic acid modifier; 40 mL/min flow rate; Wave Length: 218/326 nm; RT peak 1: 9 min, RT peak 2: 16 min
^{w}Chiral separation conditions for Example 410 and 411: Prep-Chiral HPLC Lux 5 um Cellulose-2 3 × 15 cm, 5 μm; 50%/50% EtOH/hexane with 0.1%
formic acid modifier; 40 mL/min flow rate; wave length: 218/326 nm; RT peak 1: 22.9 min; RT peak 2: 28.78 min
^{x}Isomer separation conditions for Example 412 and 413: Prep-HPLC Xselect CSH C18 OBD Column 30 × 150 mm, 5 μm; 28-44% MeCN in water with
0.1% formic acid modifier; 60 mL/min flow rate; wave length: 254 nm/220 nm nm; Peak 1 RT: 9.58 min (Example 412), Peak 2 RT: 10.73 min (Example
413)
^{y}Chiral separation conditions for example 414 and 415: Prep-Chiral HPLC CHIRALPAK IC column, 2 × 25 cm, 5 μm; 30%/70% MeOH/MTBE:Hex 1:1
with 0.1% formic acid modifier; 20 mL/min flow rate; wave length: 220/326 nm. Peak 1 RT 6.39 min (Example 414), Peak 2 RT 11.13 min (Example 415)
^{z}Chiral separation conditions for Example 417 and 418: Prep-HPLC XSelect CSH Prep C18 OBD Column, 30 × 150 mm, 5 μm; 37-56% MeCN in water
with 0.1% formic acid; 60 mL/min flow rate; wave length: 254 nm/220 nm nm. Peak 1 RT 7.88 min (Example 417), Peak 2 RT 8.72 min (Example 418)

### Example 420: Synthesis of 4-(6-methoxy-4-methyl-3,4-dihydro-2H-pyrido[3,2-b][1,4]oxazin-7-yl)-6-methyl-N-(6-(pyridin-4-yl)thiazolo[4,5-b]pyrazin-2-yl)nicotinamide

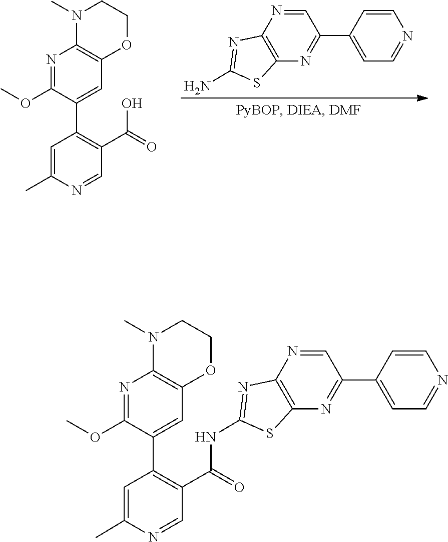 [SMILES: COc1nc2c(cc1-c1cc(C)ncc1C(=O)O)OCCN2C ; Nc1nc2ncc(-c3ccncc3)nc2s1 ; COc1nc2c(cc1-c1cc(C)ncc1C(=O)Nc1nc3ncc(-c4ccncc4)nc3s1)OCCN2C]

A solution of 4-(6-methoxy-4-methyl-3,4-dihydro-2H-pyrido[3,2-b][1,4]oxazin-7-yl)-6-methylnicotinic acid (100 mg, 317 μmol), PyBOP (198 mg, 381 μmol) and DIEA (123 mg, 951 μmol) in DMF (5 mL) was stirred at 25° C. for 1 h. 6-(pyridin-4-yl)thiazolo[4,5-b]pyrazin-2-amine (109 mg, 476 μmol) was added to the above mixture and the reaction mixture was stirred at 100° C. for 16 h. The resulting mixture was cooled to room temperature and concentrated under reduced pressure. The residue was purified by prep-TLC (DCM/MeOH 20/1) to afford crude product. The crude product was purified by prep-HPLC (25-60% MeCN in water with 1% 2 mM NH₃-MeOH modifier) to afford 4-(6-methoxy-4-methyl-3,4-dihydro-2H-pyrido[3,2-b][1,4]oxazin-7-yl)-6-methyl-N-(6-(pyridin-4-yl)thiazolo[4,5-b]pyrazin-2-yl)nicotinamide (18 mg, 10% yield) as a red solid. LCMS (ESI, m/z): 527 [M+H]⁺. ¹H NMR (400 MHz, DMSO-d₆) δ 13.51 (s, 1H), 9.45 (d, J=2.3 Hz, 1H), 8.83 (s, 2H), 8.74 (d, J=2.2 Hz, 1H), 8.45-8.23 (m, 2H), 7.48 (s, 1H), 7.22 (d, J=2.3 Hz, 1H), 4.20 (s, 2H), 3.71 (dd, J=10.7, 2.2 Hz, 1H), 3.53 (s, 4H), 3.15-2.99 (m, 3H), 2.59 (d, J=2.2 Hz, 3H).

TABLE BD
The following examples were prepared using a similar procedure to that described
for Example 420

| Ex. | | Compound | | |
|---|---|---|---|---|
| No. | Structure | Name | LCMS [M + H]⁺ | NMR |
| 421 | 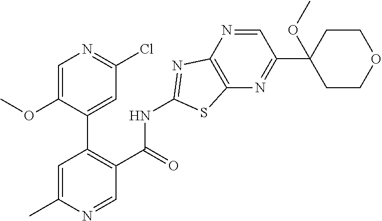 [SMILES: COc1cnc(Cl)cc1-c1cc(C)ncc1C(=O)Nc1nc2ncc(C3(OC)CCOCC3)nc2s1] | 2′-chloro-5′- methoxy-N-(6-(4- methoxytetrahydro- 2H-pyran-4- yl)thiazolo[4,5- b]pyrazin-2-yl)-6- methyl-[4,4′- bipyridine]-3- carboxamide | 527 | ¹H NMR (400 MHz, DMSO- d₆) δ 13.46 (s, 1H), 8.93 (s, 1H), 8.69 (s, 1H), 8.16 (s, 1H), 7.55 (s, 1H), 7.42 (s, 1H), 3.72-3.70 (m, 4H), 3.63 (s, 3H), 3.00 (s, 3H), 2.60 (s, 3H), 2.16-2.13 (m, 2H), 2.11- 1.99 (m, 2H) |

### Example 422: Synthesis of 2′-chloro-N-(6-(4-cyanobicyclo[2.2.2]octan-1-yl)thiazolo[4,5-b]pyrazin-2-yl)-5′-methoxy-6-methyl-[4,4′-bipyridine]-3-carboxamide

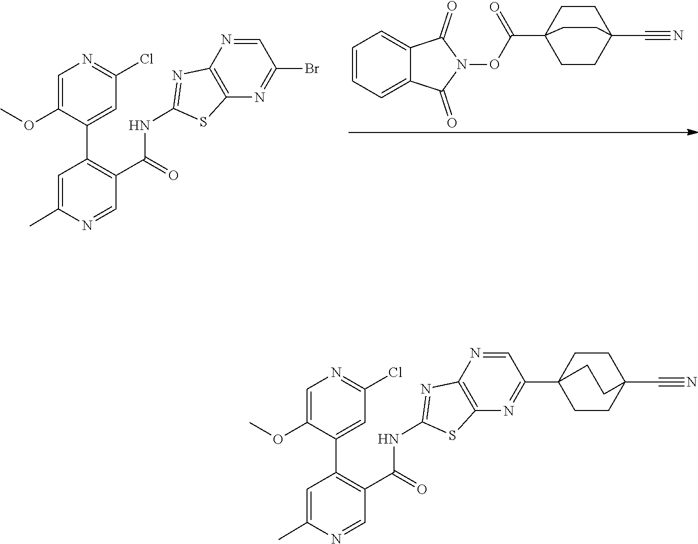

To a stirred solution of picolinimidamide hydrochloride (13 mg, 0.08 mmol) and 1-methoxy-2-(2-methoxyethoxy)ethane-dibromonickel (58 mg, 0.16 mmol) in DMA (10 mL) were added 1,3-dioxoisoindolin-2-yl 4-cyanobicyclo[2.2.2]octane-1-carboxylate (264 mg, 0.81 mmol), zinc (53 mg, 0.81 mmol) and N-(6-bromothiazolo[4,5-b]pyrazin-2-yl)-2′-chloro-5′-methoxy-6-methyl-[4,4′-bipyridine]-3-carboxamide (200 mg, 0.41 mmol) at room temperature. The resulting mixture was stirred for 16 h at 25° C. under a nitrogen atmosphere. The mixture was concentrated under reduced pressure, diluted with water (50 mL), and extracted with EtOAc (3×30 mL). The combined organic layers were washed with brine (2×20 mL) and dried over anhydrous Na₂SO₄. After filtration, the filtrate was concentrated under reduced pressure. The residue was purified by reverse-phase flash chromatography (38-55% MeCN in water with 0.1% formic acid modifier) to afford 2′-chloro-N-(6-(4-cyanobicyclo[2.2.2]octan-1-yl)thiazolo[4,5-b]pyrazin-2-yl)-5′-methoxy-6-methyl-[4,4′-bipyridine]-3-carboxamide (13 mg, 6% yield) as a white solid. LCMS (ESI, m/z): 546 [M+H]. ¹H NMR (400 MHz, DMSO-d₆) δ 13.35 (s, 1H), 8.88 (s, 1H), 8.63 (s, 1H), 8.16 (s, 1H), 7.60 (s, 1H), 7.47 (s, 1H), 3.62 (s, 3H), 2.61 (s, 3H), 2.10-1.92 (m, 12H).

TABLE BE
The following examples were prepared using a similar procedure to that described
for Example 422

| Ex. | | | |
|---|---|---|---|
| No. | Structure | Compound Name | Characterization Data |
| 423 | 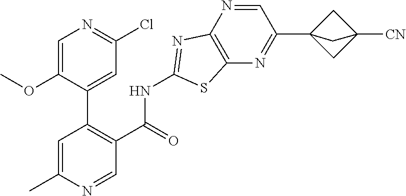 [SMILES: COc1cnc(Cl)cc1-c1cc(C)ncc1C(=O)Nc1nc2ncc(C34CC(C#N)(C3)C4)nc2s1] | 2′-chloro-N-(6-(3- cyanobicyclo[1.1.1]pentan- 1-yl)thiazolo[4,5- b]pyrazin-2-yl)-5′- methoxy-6-methyl-[4,4′- bipyridine]-3-carboxamide | LCMS (m/z): 504 [M + H]⁺. ¹H NMR (400 MHz, DMSO-d₆) δ 13.46 (s, 1H), 8.91 (s, 1H), 8.53 (s, 1H), 8.15 (s, 1H), 7.56 (s, 1H), 7.43 (s, 1H), 3.60 (s, 3H), 2.67 (s, 6H), 2.60 (s, 3H) |

### Example 424: Synthesis of 2′-chloro-N-(6-(3-(hydroxymethyl)cyclobutyl)thiazolo[4,5-b]pyrazin-2-yl)-5′-methoxy-6-methyl-[4,4′-bipyridine]-3-carboxamide

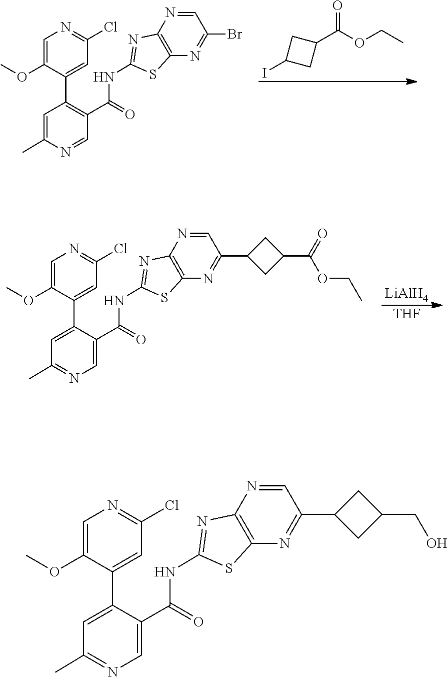

Step 1: A mixture of N-(6-bromothiazolo[4,5-b]pyrazin-2-yl)-2′-chloro-5′-methoxy-6-methyl-[4,4′-bipyridine]-3-carboxamide (200 mg, 0.4 mmol), nickel(II) dichloride (53 mg, 0.4 mmol), picolinimidamide hydrochloride (64 mg, 0.4 mmol), ethyl 3-iodocyclobutane-1-carboxylate (517 mg, 2.0 mmol), TBAI (150 mg, 0.4 mmol) and manganese (67 mg, 1.2 mmol) in DMA (15 mL) was stirred at 60° C. for 16 h under a nitrogen atmosphere. The resulting mixture was cooled to 25° C., and the mixture was purified by reverse-phase flash chromatography (10-50% MeCN in water with 0.1% formic acid modifier) to afford ethyl 3-(2-(2′-chloro-5′-methoxy-6-methyl-[4,4′-bipyridine]-3-carboxamido)thiazolo[4,5-b]pyrazin-6-yl)cyclobutane-1-carboxylate (150 mg, 55% yield) as a yellow solid. LCMS (ESI, m/z): 539 [M+H]⁺.

Step 2: To a solution of ethyl 3-(2-(2′-chloro-5′-methoxy-6-methyl-[4,4′-bipyridine]-3-carboxamido)thiazolo[4,5-b]pyrazin-6-yl)cyclobutane-1-carboxylate (100 mg, 0.2 mmol) in THF (5 mL) was added LiAlH₄ (7.0 mg, 0.2 mmol) in one portion at 0° C. under a nitrogen atmosphere. The resulting mixture was stirred at 0° C. for 1 h under a nitrogen atmosphere. The mixture was quenched with water (1 mL) and concentrated under reduced pressure. The residue was purified by reverse-phase flash chromatography (10-50% MeCN in water with 0.1% formic acid modifier) to afford 2′-chloro-N-(6-(3-(hydroxymethyl)cyclobutyl)thiazolo[4,5-b]pyrazin-2-yl)-5′-methoxy-6-methyl-[4,4′-bipyridine]-3-carboxamide (52 mg, 56% yield) as a white solid. LCMS (ESI, m/z): 497 [M+H]⁺. ¹H NMR (400 MHz, DMSO-d₆) δ 13.35 (s, 1H), 8.88 (s, 1H), 8.52 (s, 1H), 8.17 (d, J=2.0 Hz, 1H), 7.60 (s, 1H), 7.48 (s, 1H), 4.62-4.47 (m, 1H), 3.90-3.60 (m, 4H), 3.55-3.55 (m, 1H), 3.41 (t, J=5.6 Hz, 1H), 2.61 (s, 3H), 2.48-2.30 (m, 3H), 2.23-2.02 (m, 2H).

### Example 425A & 425B: Synthesis of 2′-chloro-5′-methoxy-N-(6-((cis or trans)-4-methoxy-4-methylcyclohexyl)thiazolo[4,5-b]pyrazin-2-yl)-6-methyl-[4,4′-bipyridine]-3-carboxamide (425A) & 2′-chloro-5′-methoxy-N-(6-((trans or cis)-4-methoxy-4-methylcyclohexyl)thiazolo[4,5-b]pyrazin-2-yl)-6-methyl-[4,4′-bipyridine]-3-carboxamide (425B) with unknown stereochemistry

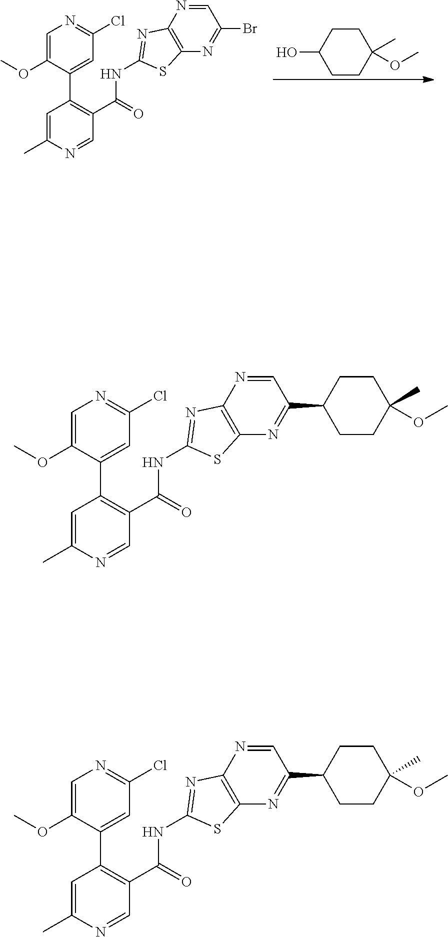

A mixture of 4-methoxy-4-methylcyclohexan-1-ol (117 mg, 0.81 mmol) and 5,7-di-tert-butyl-3-phenylbenzo[d]oxazol-3-ium tetrafluoroborate (257 mg, 0.65 mmol) in MTBE (4 mL) was stirred for 30 min at 25° C. under a nitrogen atmosphere. To the above mixture was added pyridine (51 mg, 0.65 mmol) and the resulting mixture was stirred for an additional 10 min at 25° C. To the above mixture were added N-(6-bromothiazolo[4,5-b]pyrazin-2-yl)-2′-chloro-5′-methoxy-6-methyl-[4,4′-bipyridine]-3-carboxamide (200 mg, 0.40 mmol) in DMA (4 mL), [4,4′-bis(tert-butyl)-2,2′-bipyridine]nickel dibromide (19 mg, 0.10 eq., 0.04 mmol), [Ir(dtbbpy)(ppy)₂]PF₆ (18 mg, 0.02 mmol), and quinuclidine (79 mg, 0.71 mmol). The resulting mixture was stirred for an additional 16 h at 25° C. under blue light (450 nm LED modules at 100% light intensity). The mixture was extracted with EtOAc (3×50 mL), and the combined organic layers were washed with brine (1×5 mL) and dried over anhydrous Na₂SO₄. After filtration, the filtrate was concentrated under reduced pressure. The residue was purified by reverse-phase flash chromatography (25-50% MeOH in water with NH₃·H₂O modifier) to afford 2′-chloro-5′-methoxy-N-(6-(4-methoxy-4-methylcyclohexyl)thiazolo[4,5-b]pyrazin-2-yl)-6-methyl-[4,4′-bipyridine]-3-carboxamide (180 mg, 80% purity) as a mixture of isomers. The isomeric mixture was purified by Prep-HPLC (Xselect CSH C18 OBD Column 30×150 mm, 5 μm; 37-57% MeCN in water with 0.1% formic acid modifier) to afford two compounds:

Peak 1 RT=8.62 min: 2′-chloro-5′-methoxy-N-(6-((cis or trans)-4-methoxy-4-methylcyclohexyl)thiazolo[4,5-b]pyrazin-2-yl)-6-methyl-[4,4′-bipyridine]-3-carboxamide (40 mg, 92% purity) which was further purified by Prep-HPLC (XSelect CSH Fluoro Phenyl column, 30×150 mm, 5 μm; 56-76% MeOH in water with 0.1% formic acid modifier) to afford 2′-chloro-5′-methoxy-N-(6-((cis or trans)-4-methoxy-4-methylcyclohexyl)thiazolo[4,5-b]pyrazin-2-yl)-6-methyl-[4,4-bipyridine]-3-carboxamide (18 mg, 1300 yield) as a white solid. LCMS (ESI, m/z): 539 [M+H]. ¹HNMR (400 MHz, DMSO-d₆): δ13.34 (s, 1H), 8.88 (s, 1H), 8.54 (s, 1H), 8.16 (s, 1H), 7.58 (s, 1H), 7.45 (s, 1H), 3.62 (s, 3H), 3.11 (s, 3H), 2.88-2.77 (m, 1H), 2.60 (s, 3H), 1.94-1.74 (l, 4H), 1.69-1.59 (m, 2H), 1.46-1.35 (m, 2H), 1.11 (s, 3H).

Peak 2 RT=9.92 min: 2′-chloro-5′-methoxy-N-(6-((trans or cis)-4-methoxy-4-methylcyclohexyl)thiazolo[4,5-b]pyrazin-2-yl)-6-methyl-[4,4′-bipyridine]-3-carboxamide (24 mg, 16% yield) as a white solid. LCMS (ESI, m/z): 539 [M+H]⁺. ¹H NMR (400 MHz, DMSO-d₆): δ 13.34 (s, 1H), 8.88 (s, 1H), 8.60 (s, 1H), 8.17 (s, 1H), 7.60 (s, 1H), 7.48 (s, 1H), 3.62 (s, 3H), 3.16 (s, 3H), 2.99-2.86 (m, 1H), 2.61 (s, 3H), 1.95-1.85 (m, 2H), 1.82-1.66 (m, 4H), 1.62-1.48 (i, 2H), 1.21 (s, 3H).

TABLE BF
The following examples were prepared using a similar procedure to that described
for Example 425A and 425B

| Ex. | | Compound | |
|---|---|---|---|
| No. | Structure | Name | Characterization Data |
| 426^{aa} | 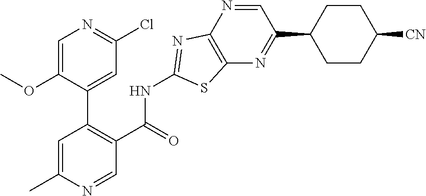 [SMILES: COc1cnc(Cl)cc1-c1cc(C)ncc1C(=O)Nc1nc2ncc([C@H]3CC[C@@H](C#N)CC3)nc2s1] | 2′-chloro-N- (6-((1s,4s)-4- cyanocyclo- hexyl)thiazolo [4,5-b]pyrazin- 2-yl)-5′- methoxy-6- methyl-[4,4′- bipyridine]-3- carboxamide | LCMS (m/z): 520 [M + H]⁺. ¹H NMR (400 MHz, DMSO-d₆) δ 13.38 (s, 1H), 8.89 (s, 1H), 8.57 (s, 1H), 8.17 (s, 1H), 7.60 (s, 1H), 7.48 (s, 1H), 3.62 (s, 3H), 3.28- 3.22 (m, 1H), 3.01-2.92 (m, 1H), 2.61 (s, 3H), 2.04-1.96 (m, 2H), 1.96-1.88 (m, 2H), 1.86-1.70 (m, 4H) |
| 427^{aa} | 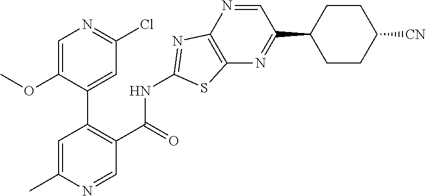 [SMILES: COc1cnc(Cl)cc1-c1cc(C)ncc1C(=O)Nc1nc2ncc([C@H]3CC[C@H](C#N)CC3)nc2s1] | 2′-chloro-N- (6-((1r,4r)-4- cyanocyclo- hexyl)thiazolo [4,5-b]pyrazin- 2-yl)-5′- methoxy-6- methyl-[4,4′- bipyridine]-3- carboxamide | LCMS (m/z): 520 [M + H]⁺. ¹H NMR (400 MHz, DMSO-d₆) δ 13.39 (s, 1H), 8.88 (s, 1H), 8.55 (s, 1H), 8.17 (s, 1H), 7.60 (s, 1H), 7.47 (s, 1H), 3.62 (s, 3H), 3.01- 2.90 (m, 1H), 2.88-2.75 (m, 1H), 2.61 (s, 3H), 2.20-2.08 (m, 2H), 2.01-1.87 (m, 2H), 1.74-1.59 (m, 4H) |
| 428 | 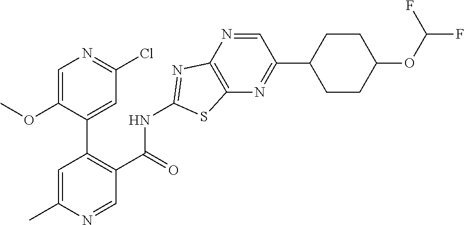 [SMILES: COc1cnc(Cl)cc1-c1cc(C)ncc1C(=O)Nc1nc2ncc(C3CCC(OC(F)F)CC3)nc2s1] | 2′-chloro-N- (6-(4-(difluoro- methoxy)cyclo- hexyl)thiazolo [4,5-b]pyrazin- 2-yl)-5′- methoxy-6- methyl-[4,4′- bipyridine]-3- carboxamide | LCMS (m/z): 561 [M + H]⁺. ¹H NMR (400 MHz, DMSO-d₆) δ 13.35 (s, 1H), 8.89 (s, 1H), 8.55 (d, J = 2.5 Hz, 1H), 8.16 (s, 1H), 7.59 (s, 1H), 7.47 (s, 1H), 6.93- 6.55 (m, 1H), 4.45 (d, J = 3.9 Hz, 1H), 3.62 (d, J = 1.9 Hz, 3H), 3.04- 2.92 (m, 1H), 2.61 (s, 3H), 2.01- 1.85 (m, 4H), 1.79-1.65 (m, 4H) |
| 429^{%,ab} | 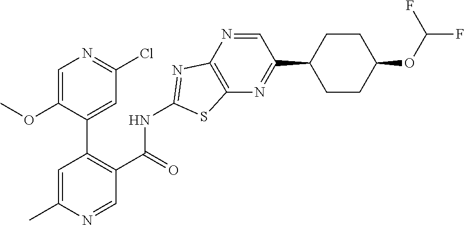 [SMILES: COc1cnc(Cl)cc1-c1cc(C)ncc1C(=O)Nc1nc2ncc([C@H]3CC[C@@H](OC(F)F)CC3)nc2s1] | 2′-chloro-N- (6-((cis or trans)-4- (difluoro- methoxy)cyclo- hexyl)thiazolo [4,5-b]pyrazin- 2-yl)-5′- methoxy-6- methyl-[4,4′- bipyridine]-3- carboxamide | LCMS (m/z): 561 [M + H]⁺. ¹H NMR (400 MHz, DMSO-d₆) δ 13.36 (s, 1H), 8.87 (s, 1H), 8.58 (s, 1H), 8.17 (s, 1H), 7.60 (s, 1H), 7.48 (s, 1H), 6.74 (t, J = 76.7 Hz, 1H), 4.21-4.09 (m, 1H), 3.62 (s, 3H), 2.91 (tt, J = 11.9, 3.6 Hz, 1H), 2.61 (s, 3H), 2.15-2.04 (m, 2H), 1.98 (d, J = 13.0 Hz, 2H), 1.72 (qd, J = 13.3, 3.1 Hz, 2H), 1.63-1.49 (m, 2H) |
| 430^{%,ab} | 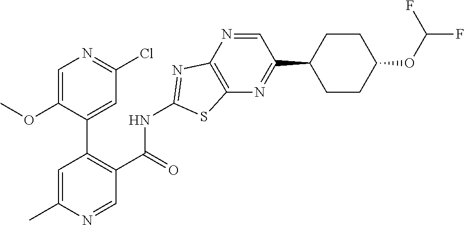 [SMILES: COc1cnc(Cl)cc1-c1cc(C)ncc1C(=O)Nc1nc2ncc([C@H]3CC[C@H](OC(F)F)CC3)nc2s1] | 2′-chloro-N-(6- ((trans or cis)- 4-(difluoro- methoxy)cyclo- hexyl)thiazolo [4,5-b]pyrazin- 2-yl)-5′- methoxy-6- methyl-[4,4′- bipyridine]-3- carboxamide | LCMS (m/z): 561 [M + H]⁺. ¹H NMR (400 MHz, DMSO-d₆) δ 13.33 (s, 1H), 8.88 (s, 1H), 8.56 (s, 1H), 8.16 (s, 1H), 7.58 (s, 1H), 7.47 (s, 1H), 6.74 (t, J = 76.8 Hz, 1H), 4.45 (t, J = 3.0 Hz, 1H), 3.63 (s, 3H), 3.04-2.93 (m, 1H), 2.61 (s, 3H), 2.00-1.82 (m, 4H), 1.81- 1.66 (m, 4H) |
| 431^{%,ac} | 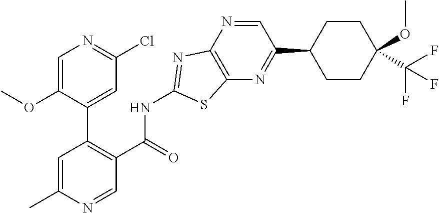 [SMILES: COc1cnc(Cl)cc1-c1cc(C)ncc1C(=O)Nc1nc2ncc([C@H]3CC[C@](OC)(C(F)(F)F)CC3)nc2s1] | 2′-chloro-5′- methoxy-N-(6- ((cis or trans)-4- methoxy-4- (trifluoromethyl) cyclohexyl) thiazolo[4,5-b] pyrazin-2-yl)-6- methyl-[4,4′- bipyridine]-3- carboxamide | LCMS (m/z): 593 [M + H]⁺. ¹H NMR (400 MHz, Methanol-d₄) δ 8.85 (s, 1H), 8.56 (s, 1H), 8.07 (s, 1H), 7.49 (d, J = 27.7 Hz, 2H), 3.71 (s, 3H), 3.44 (s, 3H), 3.26 (d, J = 5.0 Hz, 1H), 2.69 (s, 3H), 2.23- 1.96 (m, 6H), 1.90-1.79 (m, 2H) |
| 432^{%,ac} | 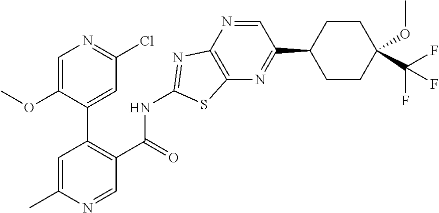 [SMILES: COc1cnc(Cl)cc1-c1cc(C)ncc1C(=O)Nc1nc2ncc([C@H]3CC[C@@](OC)(C(F)(F)F)CC3)nc2s1] | 2′-chloro-5′- methoxy-N-(6- ((trans or cis)-4- methoxy-4- (trifluoromethyl) cyclohexyl) thiazolo[4,5-b] pyrazin-2-yl)-6- methyl-[4,4′- bipyridine]-3- carboxamide | LCMS (m/z): 593 [M + H]⁺. ¹H NMR (400 MHz, Methanol-d₄) δ 8.85 (s, 1H), 8.49 (s, 1H), 8.07 (s, 1H), 7.49 (d, J = 28.3 Hz, 2H), 3.71 (s, 3H), 3.47 (d, J = 1.4 Hz, 3H), 2.98 (tt, J = 11.8, 4.0 Hz, 1H), 2.69 (s, 3H), 2.16 (dd, J = 14.3, 2.8 Hz, 2H), 2.02-1.84 (m, 4H), 1.77 (td, J = 13.5, 4.1 Hz, 2H) |
| 433^{%,ad} | 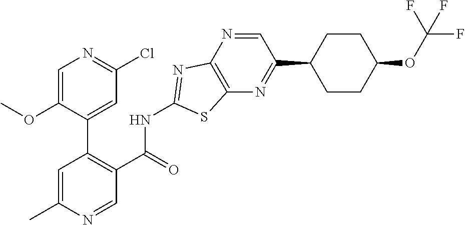 [SMILES: COc1cnc(Cl)cc1-c1cc(C)ncc1C(=O)Nc1nc2ncc([C@H]3CC[C@@H](OC(F)(F)F)CC3)nc2s1] | 2′-chloro-5′- methoxy-6- methyl-N-(6- ((cis or trans)-4- (trifluoro- methoxy)cyclo- hexyl)thiazolo [4,5-b]pyrazin- 2-yl)-[4,4′- bipyridine]-3- carboxamide | LCMS (m/z): 579 [M + H]⁺. ¹H NMR (400 MHz, DMSO-d₆) δ 13.35 (s, 1H), 8.87 (s, 1H), 8.59 (d, J = 1.1 Hz, 1H), 8.17 (d, J = 1.0 Hz, 1H), 7.60 (d, J = 1.1 Hz, 1H), 7.48 (s, 1H), 4.76 (s, 1H), 3.62 (s, 3H), 3.10-2.97 (m, 1H), 2.61 (s, 3H), 2.02 (d, J = 12.0 Hz, 2H), 1.95-1.76 (m, 6H) |
| 434^{%,ad} | 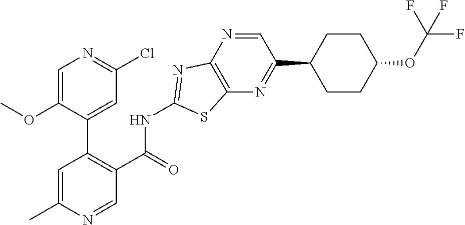 [SMILES: COc1cnc(Cl)cc1-c1cc(C)ncc1C(=O)Nc1nc2ncc([C@H]3CC[C@H](OC(F)(F)F)CC3)nc2s1] | 2′-chloro-5′- methoxy-6- methyl-N-(6- ((trans or cis)-4- (trifluoro- methoxy)cyclo- hexyl)thiazolo [4,5-b]pyrazin-2- yl)-[4,4′- bipyridine]-3- carboxamide | LCMS (m/z): 579 [M + H]⁺. ¹H NMR (400 MHz, DMSO-d₆) δ 13.37 (s, 1H), 8.88 (s, 1H), 8.57 (s, 1H), 8.16 (s, 1H), 7.59 (s, 1H), 7.47 (s, 1H), 4.54-4.42 (m, 1H), 3.62 (s, 3H), 3.00-2.86 (m, 1H), 2.61 (s, 3H), 2.21-2.10 (m, 2H), 1.99 (d, J = 12.8 Hz, 2H), 1.85- 1.61 (m, 4H) |
| 435 | 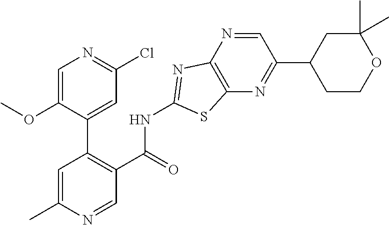 | 2′-chloro-N- (6-(2,2- dimethyltetra- hydro-2H-pyran- 4-yl)thiazolo [4,5-b]pyrazin-2- yl)-5′-methoxy- 6-methyl-[4,4′- bipyridine]-3- carboxamide | LCMS (m/z): 525 [M + H]⁺. ¹H NMR (400 MHz, DMSO-d₆) δ 13.38 (s, 1H), 8.88 (s, 1H), 8.60 (s, 1H), 8.17 (s, 1H), 7.61 (s, 1H), 7.49 (s, 1H), 3.73 (dd, J = 8.7, 2.5 Hz, 2H), 3.64-3.59 (m, 3H), 3.33- 3.19 (m, 1H), 2.61 (s, 3H), 1.93- 1.53 (m, 4H), 1.28 (s, 3H), 1.19 (s, 3H) |
| 436^{ae} | 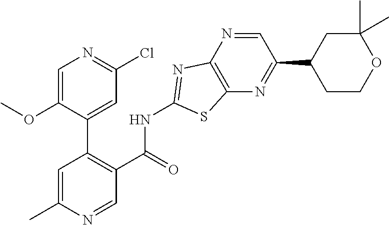 | (S or R)-2′- chloro-N-(6- (2,2-dimethyl- tetrahydro-2H- pyran-4-yl) thiazolo[4,5-b] pyrazin-2-yl)-5′- methoxy-6- methyl-[4,4′- bipyridine]-3- carboxamide | LCMS (m/z): 525 [M + H]⁺. ¹H NMR (400 MHz, DMSO-d₆) δ 13.39 (s, 1H), 8.88 (s, 1H), 8.60 (s, 1H), 8.17 (s, 1H), 7.60 (s, 1H), 7.48 (s, 1H), 3.73 (dd, J = 8.7, 2.5 Hz, 2H), 3.62 (s, 3H), 3.37 (s, 1H), 2.61 (s, 3H), 1.83-1.56 (m, 4H), 1.27 (d, J = 6.4 Hz, 3H), 1.19 (s, 3H). |
| 437^{ae} | 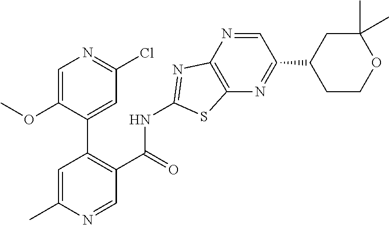 | (R or S)-2′- chloro-N-(6- (2,2-dimethyl- tetrahydro-2H- pyran-4-yl) thiazolo[4,5-b] pyrazin-2-yl)-5′- methoxy-6- methyl-[4,4′- bipyridine]-3- carboxamide | LCMS (m/z): 525 [M + H]⁺. ¹H NMR (400 MHz, DMSO-d₆) δ 13.34 (s, 1H), 8.90 (s, 1H), 8.55 (s, 1H), 8.16 (s, 1H), 7.57 (s, 1H), 7.44 (s, 1H), 3.73 (dd, J = 8.5, 2.5 Hz, 2H), 3.62 (s, 3H), 3.43 (s, 1H), 2.60 (s, 3H), 1.81-1.58 (m, 4H), 1.28 (s, 3H), 1.19 (s, 3H) |
| 438 | 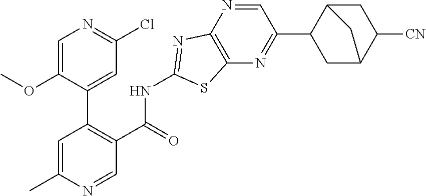 [SMILES: COc1cnc(Cl)cc1-c1cc(C)ncc1C(=O)Nc1nc2ncc(C3CC4CC3CC4C#N)nc2s1] | 2′-chloro-N- (6-(5-cyano- bicyclo[2.2.1] heptan-2-yl) thiazolo[4,5-b] pyrazin-2-yl)-5′- methoxy-6- methyl-[4,4′- bipyridine]-3- carboxamide | LCMS (m/z): 532 [M + H]⁺. ¹H NMR (400 MHz, DMSO-d₆) δ 13.36 (s, 1H), 8.88 (s, 1H), 8.60 (d, J = 16.6 Hz, 1H), 8.16 (s, 1H), 7.60 (s, 1H), 7.48 (s, 1H), 3.62 (s, 3H), 3.21 (t, J = 7.1 Hz, 1H), 3.07 (m, 1H), 2.67 (d, J = 5.1 Hz, 1H), 2.61 (s, 3H), 2.48-2.45 (m, 1H), 2.13-1.95 (m, 2H), 1.87-1.73 (m, 2H), 1.59 (d, J = 12.2 Hz, 1H), 1.30-1.21 (m, 1H) |
| 439^{%,af} | 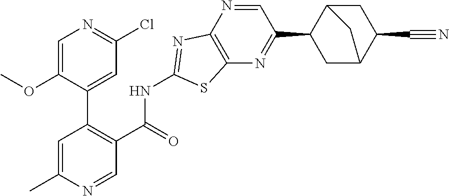 [SMILES: COc1cnc(Cl)cc1-c1cc(C)ncc1C(=O)Nc1nc2ncc([C@@H]3CC4CC3C[C@H]4C#N)nc2s1] | 2′-chloro-N- (6-((2R or S,5R or S)-5-cyano- bicyclo[2.2.1] heptan-2-yl) thiazolo[4,5-b] pyrazin-2-yl)-5′- methoxy-6- methyl-[4,4′- bipyridine]-3- carboxamide | LCMS (m/z): 532 [M + H]⁺. ¹H NMR (400 MHz, DMSO-d₆) δ 13.36 (s, 1H), 8.88 (s, 1H), 8.57 (s, 1H), 8.16 (s, 1H), 7.59 (s, 1H), 7.47 (s, 1H), 3.61 (s, 3H), 3.12- 3.05 (m, 1H), 2.83-2.77 (m, 1H), 2.69 (d, J = 4.4 Hz, 1H), 2.61 (s, 3H), 2.14-2.06 (m, 1H), 2.03- 1.94 (m, 1H), 1.88-1.73 (m, 3H), 1.40 (d, J = 10.4 Hz, 1H), 1.25- 1.21 (m, 1H) |
| 440^{%,af} | 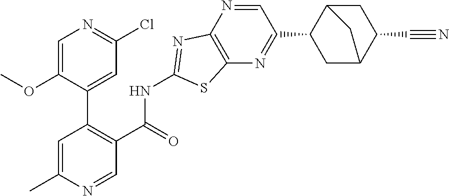 [SMILES: COc1cnc(Cl)cc1-c1cc(C)ncc1C(=O)Nc1nc2ncc([C@H]3CC4CC3C[C@@H]4C#N)nc2s1] | 2′-chloro-N- (6-((2S or R, 5S or R)-5- cyanobicyclo [2.2.1]heptan- 2-yl)thiazolo [4,5-b]pyrazin- 2-yl)-5′- methoxy-6- methyl-[4,4′- bipyridine]-3- carboxamide | LCMS (m/z): 532 [M + H]⁺. ¹H NMR (400 MHz, DMSO-d₆) δ 13.35 (s, 1H), δ 8.89 (s, 1H), 8.56 (s, 1H), 8.16 (s, 1H), 7.58 (s, 1H), 7.46 (s, 1H), 3.61 (d, J = 1.9 Hz, 3H), 3.12-3.05 (m, 1H), 2.82- 2.76 (m, 1H), 2.68 (s, 1H), 2.60 (s, 3H), 2.13-2.05 (m, 1H), 2.04- 1.94 (m, 1H), 1.88-1.71 (m, 3H), 1.40 (d, J = 10.3 Hz, 1H), 1.26- 1.20 (m, 1H) |
| 441^{%,af} | 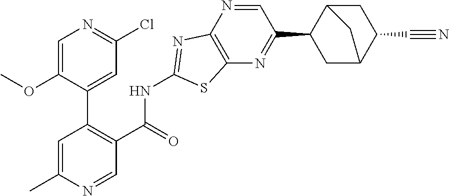 [SMILES: COc1cnc(Cl)cc1-c1cc(C)ncc1C(=O)Nc1nc2ncc([C@@H]3CC4CC3C[C@@H]4C#N)nc2s1] | 2′-chloro-N- (6-((2R or S, 5S or R)-5- cyanobicyclo [2.2.1]heptan-2- yl)thiazolo[4,5- b]pyrazin-2-yl)- 5′-methoxy-6- methyl-[4,4′- bipyridine]-3- carboxamide | LCMS (m/z): 532 [M + H]⁺. ¹H NMR (400 MHz, DMSO-d₆) δ 13.35 (s, 1H), 8.88 (s, 1H), 8.62 (s, 1H), 8.16 (s, 1H), 7.60 (s, 1H), 7.48 (s, 1H), 3.62 (s, 3H), 3.26- 3.17 (m, 1H), 3.08-3.01 (m, 1H), 2.67 (d, J = 4.1 Hz, 1H), 2.61 (s, 3H), 2.47 (d, J = 4.2 Hz, 1H), 2.19- 2.03 (m, 3H), 1.85 (d, J = 10.1 Hz, 1H), 1.63-1.56 (m, 1H), 1.30- 1.20 (m, 1H) |
| 442^{%,af} | 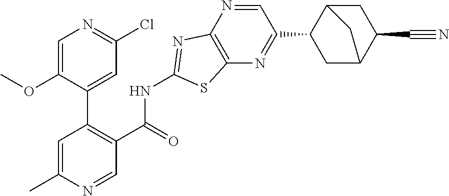 [SMILES: COc1cnc(Cl)cc1-c1cc(C)ncc1C(=O)Nc1nc2ncc([C@H]3CC4CC3C[C@H]4C#N)nc2s1] | 2′-chloro-N- (6-((2S or R, 5R or S)-5- cyanobicyclo [2.2.1]heptan-2- yl)thiazolo[4,5- b]pyrazin-2-yl)- 5′-methoxy-6- methyl-[4,4′- bipyridine]-3- carboxamide | LCMS (m/z): 532 [M + H]⁺. ¹H NMR (400 MHz, DMSO-d₆) δ 13.35 (s, 1H), 8.88 (s, 1H), 8.62 (s, 1H), 8.16 (s, 1H), 7.60 (s, 1H), 7.47 (s, 1H), 3.62 (s, 3H), 3.21 (dd, J = 8.6, 5.6 Hz, 1H), 3.08- 3.01 (m, 1H), 2.67 (d, J = 4.1 Hz, 1H), 2.61 (s, 3H), 2.47 (d, J = 4.2 Hz, 1H), 2.19-2.04 (m, 3H), 1.85 (d, J = 10.0 Hz, 1H), 1.65-1.54 (m, 1H), 1.31-1.20 (m, 1H) |

^{%}Unknown stereochemistry
^{aa}Chiral separation conditions for Example 426 and 427: Prep-SFC (CHIRALPAK IH, 3.0 × 50 mm, 3 μm column; 40%/60% MeOH/CO₂, 100 mL/min
flow rate; RT peak 1: 4.17 min; RT peak 2: 6.05 min; Sample Solvent: DCM--HPLC; Injection Volume: 4 mL; Number Of Runs: 7)
^{ab}Chiral separation conditions for Example 429 and 430: Prep-Chiral HPLC (CHIRALPAK ID, 3 × 25 cm, 5 μm column; 70%/30% 1:1 MTBE:Hex with
(0.1% formic acid modifier)/MeOH; 40 mL/min flow rate; RT peak 1: 7 min; RT peak 2: 12 min; Sample Solvent: DCM--HPLC; Injection Volume: 3 mL;
Number Of Runs: 5)
^{ac}Chiral separation conditions for Example 431 and 432: Prep-Chiral HPLC (CHIRALFLASH IA, 20 um 30 mm × 100 mm column; 90%/10% 1:1 MTBE:
Hex with 0.1% DEA modifier/MeOH; 20 mL/min flow rate; RT peak 1: 10.1 min; RT peak 2: 13.7 min; Sample Solvent: MeOH: DCM = 1: 1--HPLC;
Injection Volume: 0.6 mL; Number of Runs 4
^{ad}Chiral separation conditions for Example 433 and 434: Prep-Chiral HPLC Xselect CSH C18 OBD Column 30 × 150 mm 5 μm, 50-65% MeCN in water
0.1% formic acid modifier; 60 mL/min flow rate; Wave Length: 254/220 nm; RT peak 1: 8.93 min; RT peak 2: 9.57 min
^{ae}Chiral separation conditions for Example 436 and 437: Prep-Chiral HPLC CHIRALPAK IA column, 3 × 25 cm, 5 μm; 30%/70% MeOH/MTBE:Hex
1:1 with 0.1% formic acid modifier; 40 mL/min flow rate; wave length: 218/328 nm; Peak 1 RT: 7 min (Example 436), Peak 2 RT: 14 min (Example 437)
^{af}Chiral separation conditions for Example 439-442: Prep-Chiral HPLC CHIRAL ART Cellulose-SC column, 3 × 25 cm, 5 μm, 30%/70% MeOH in MTBE
with 0.1% formic acid modifier; 40 mL/min flow rate; Wave Length: 218/328 nm to afford three peaks: Peak 1 as a mixture of Example 439 and 440;
Peak 2: 2′-chloro-N-(6-((2R,5S)-5-cyanobicyclo[2.2.1]heptan-2-yl)thiazolo[4,5-b]pyrazin-2-yl)-5′-methoxy-6-methyl-[4,4′-bipyridine]-3-carboxamide
(Example 441, 20 mg, 33%) as a white solid; Peak 3: 2′-chloro-N-(6-((2S,5R)-5-cyanobicyclo[2.2.1]heptan-2-yl)thiazolo[4,5-b]pyrazin-2-yl)-5′-methoxy-
6-methyl-[4,4′-bipyridine]-3-carboxamide (Example 442, 20 mg, 33%) as white solid. Peak 1 (mixture of Example 439 and 440) was repurified by Prep-
Chiral-HPLC Lux Cellulose-4 column 3 × 25 cm, 5 μm; 50%/50% 5:1 EtOH:MeCN/Hexane with 0.1% formic acid modifier; 40 mL/min flow rate;
wave length: 218/328 nm to afford Example 439 (RT: 9.475 min) and Example 440 (12.35 min)

### Example 443: Synthesis of 2′-chloro-5′-methoxy-6-methyl-N-(6-(1-(2,2,2-trifluoroethyl)azetidin-3-yl)thiazolo[4,5-b]pyrazin-2-yl)-[4,4′-bipyridine]-3-carboxamide

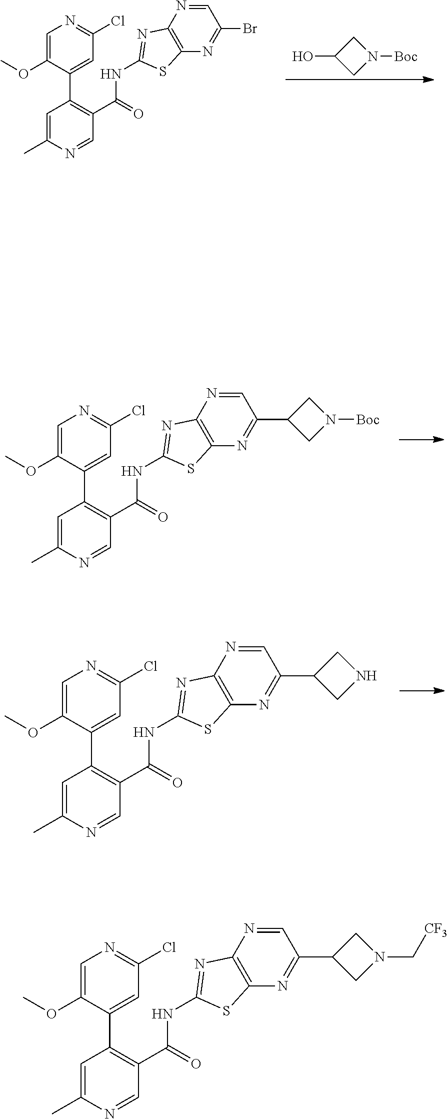 [SMILES: COc1cnc(Cl)cc1-c1cc(C)ncc1C(=O)Nc1nc2ncc(Br)nc2s1 ; CC(C)(C)OC(=O)N1CC(O)C1 ; COc1cnc(Cl)cc1-c1cc(C)ncc1C(=O)Nc1nc2ncc(C3CN(C(=O)OC(C)(C)C)C3)nc2s1 ; COc1cnc(Cl)cc1-c1cc(C)ncc1C(=O)Nc1nc2ncc(C3CNC3)nc2s1 ; COc1cnc(Cl)cc1-c1cc(C)ncc1C(=O)Nc1nc2ncc(C3CN(CC(F)(F)F)C3)nc2s1]

Step 1: Synthesized as described in Example 425A with tert-butyl 3-hydroxyazetidine-1-carboxylate to afford tert-butyl 3-(2-(2′-chloro-5′-methoxy-6-methyl-[4,4′-bipyridine]-3-carboxamido)thiazolo[4,5-b]pyrazin-6-yl)azetidine-1-carboxylate (96 mg, 21% yield) as a yellow solid. LCMS (ESI, m/z): 568 [M+H]⁺. ¹H NMR (400 MHz, DMSO-d₆) δ 13.50 (s, 1H), 8.95 (s, 1H), 8.62 (d, J=2.6 Hz, 1H), 8.22 (d, J=2.3 Hz, 1H), 7.66 (d, J=2.6 Hz, 1H), 7.53 (d, J=3.1 Hz, 1H), 4.23-4.03 (m, 4H), 3.68 (d, J=2.4 Hz, 4H), 2.67 (s, 3H), 1.47 (s, 9H).

Step 2: To a stirred solution of tert-butyl 3-(2-(2′-chloro-5′-methoxy-6-methyl-[4,4′-bipyridine]-3-carboxamido)thiazolo[4,5-b]pyrazin-6-yl)azetidine-1-carboxylate (90 mg, 0.16 mmol) in DCM (3 mL) was added TFA (1 mL) dropwise at room temperature under a nitrogen atmosphere. The reaction was stirred 1 h at 25° C. under a nitrogen atmosphere. The resulting mixture was concentrated under reduced pressure to afford N-(6-(azetidin-3-yl)thiazolo[4,5-b]pyrazin-2-yl)-2′-chloro-5′-methoxy-6-methyl-[4,4′-bipyridine]-3-carboxamide (55 mg, crude) as an off-white solid. The crude product was used in the next step directly without further purification. LCMS (ESI, m/z): 468 [M+H]⁺.

Step 3: To a stirred solution of N-(6-(azetidin-3-yl)thiazolo[4,5-b]pyrazin-2-yl)-2′-chloro-5′-methoxy-6-methyl-[4,4′-bipyridine]-3-carboxamide (60 mg, 0.13 mmol, TFA salt) and 2,2,2-trifluoroethyl trifluoromethylsulfonate (30 mg, 0.13 mmol) in THF (2 mL) was added TEA (52 mg, 0.51 mmol) in portions at room temperature under a nitrogen atmosphere. The reaction was stirred overnight at 80° C. under a nitrogen atmosphere. The mixture was cooled to room temperature and concentrated under reduced pressure. The residue was purified by Prep-TLC (CH₂Cl₂/CH₃OH=15:1) to afford crude product, which was further purified by Prep-HPLC (52-72% MeOH in water with 0.1% formic acid modifier) to afford 2′-chloro-5′-methoxy-6-methyl-N-(6-(1-(2,2,2-trifluoroethyl)azetidin-3-yl)thiazolo[4,5-b]pyrazin-2-yl)-[4,4′-bipyridine]-3-carboxamide (1.6 mg, 2.2% yield) as a white solid. LCMS (ESI, m/z): 550 [M+H]⁺. ¹H NMR (400 MHz, DMSO-d₆) δ 13.41 (brs, 1H), 8.91 (s, 1H), 8.57 (s, 1H), 8.16 (s, 1H), 7.57 (s, 1H), 7.44 (s, 1H), 4.03-4.01 (m, 1H), 3.82 (t, J=7.3 Hz, 2H), 3.62 (s, 3H), 3.58 (t, J=7.1 Hz, 2H), 3.29 (d, J=10.2 Hz, 2H), 2.60 (s, 3H).

### Example 444, 445A, & 445B: Synthesis of 2′-chloro-N-(6-(4-hydroxycyclohexyl)thiazolo[4,5-b]pyrazin-2-yl)-5′-methoxy-6-methyl-[4,4′-bipyridine]-3-carboxamide (444); (cis or trans)-2′-chloro-N-(6-(4-hydroxycyclohexyl)thiazolo[4,5-b]pyrazin-2-yl)-5′-methoxy-6-methyl-[4,4′-bipyridine]-3-carboxamide (445A); & (trans or cis)-2′-chloro-N-(6-(4-hydroxycyclohexyl)thiazolo[4,5-b]pyrazin-2-yl)-5′-methoxy-6-methyl-[4,4′-bipyridine]-3-carboxamide (445B)

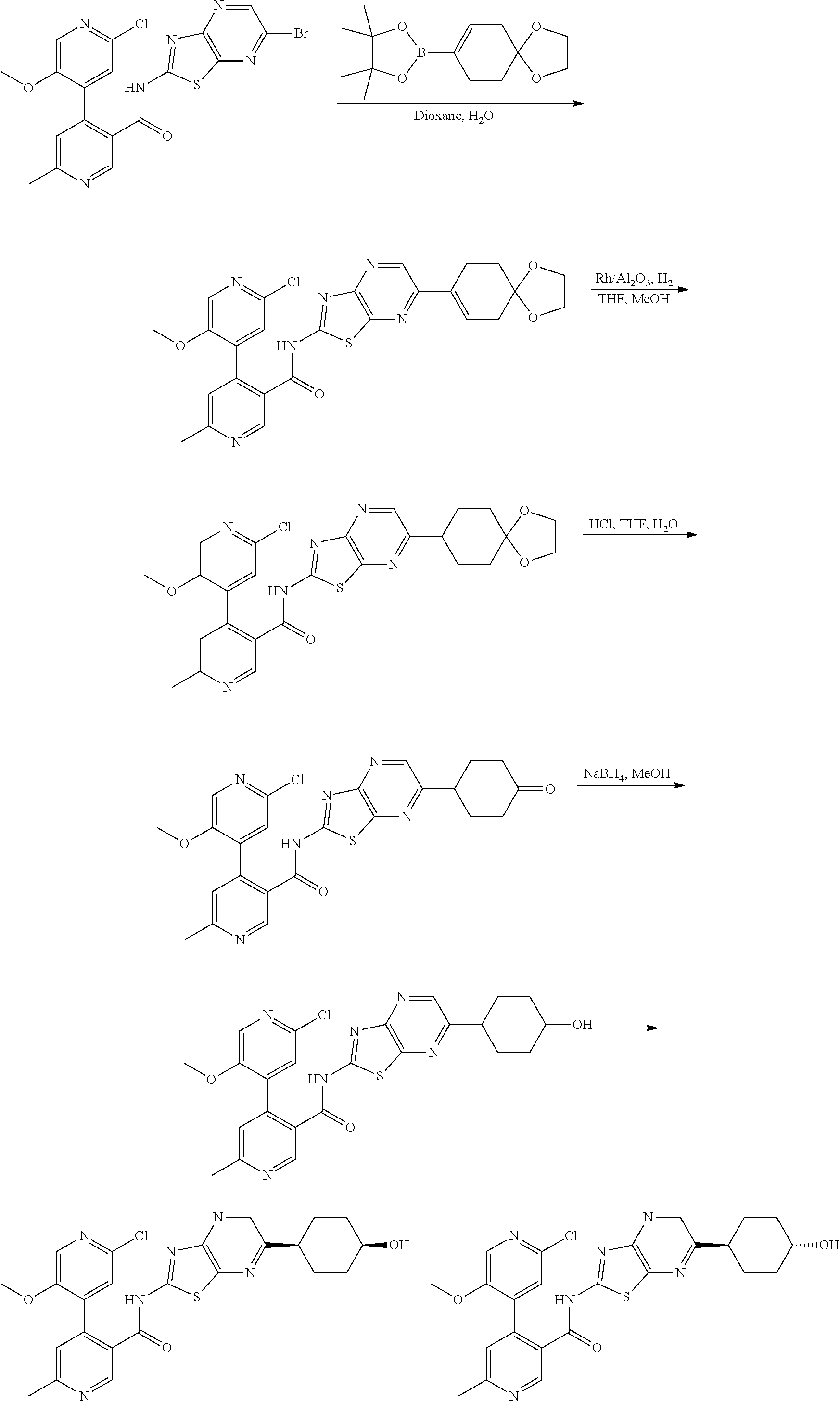

Step 1: To a stirred solution of N-(6-bromothiazolo[4,5-b]pyrazin-2-yl)-2′-chloro-5′-methoxy-6-methyl-[4,4′-bipyridine]-3-carboxamide (1.0 g, 2.0 mmol) and 4,4,5,5-tetramethyl-2-(1,4-dioxaspiro[4.5]dec-7-en-8-yl)-1,3,2-dioxaborolane (600 mg, 2.0 mmol) in 1,4-dioxane (20 mL) and water (2 mL) were added PdCl₂(PPh₃)₂ (300 mg, 0.4 mmol) and cesium carbonate (2.0 g, 6.0 mmol). The mixture was charged with N₂ three times and stirred at 90° C. for 12 h under a N₂ atmosphere. The resulting mixture was cooled to 25° C. and concentrated under reduced pressure. The residue was purified by reverse phase chromatography (40-60% ACN in water with 0.1% formic acid modifier) to afford N-(6-(1,4-dioxaspiro[4.5]dec-7-en-8-yl)thiazolo[4,5-b]pyrazin-2-yl)-2′-chloro-5′-methoxy-6-methyl-[4,4′-bipyridine]-3-carboxamide (509 mg, 50% yield) as a yellow solid. LCMS (ESI, m/z): 551 [M+H]⁺.

Step 2: To a solution of N-(6-(1,4-dioxaspiro[4.5]dec-7-en-8-yl)thiazolo[4,5-b]pyrazin-2-yl)-2′-chloro-5′-methoxy-6-methyl-[4,4′-bipyridine]-3-carboxamide (410 mg, 0.7 mmol) in THF (10 mL) and MeOH (10 mL) was added Rh/Al₂O₃ (187 mg, 0.2 mmol). The mixture was charged with H₂ three times and stirred at 50° C. for 6 h under a H₂ atmosphere. The resulting mixture was cooled to 25° C. After filtration, the filter cake was washed with MeOH (3×20 mL). The combined filtrates were concentrated under reduced pressure. The residue was purified by silica gel chromatography (0-60% EtOAc in PE) to afford N-(6-(1,4-dioxaspiro[4.5]decan-8-yl)thiazolo[4,5-b]pyrazin-2-yl)-2′-chloro-5′-methoxy-6-methyl-[4,4′-bipyridine]-3-carboxamide (358 mg, 87% yield) as a yellow solid. LCMS (ESI, m/z): 553 [M+H]⁺.

Step 3: To a stirred solution of N-(6-(1,4-dioxaspiro[4.5]decan-8-yl)thiazolo[4,5-b]pyrazin-2-yl)-2′-chloro-5′-methoxy-6-methyl-[4,4′-bipyridine]-3-carboxamide (380 mg, 0.7 mmol) in THF (1 mL) and water (1 mL) were added HCl (0.1 mL, 1N) dropwise. The mixture was stirred at 25° C. for 2 h. The reaction mixture was quenched with saturated aqueous NaHCO₃ solution (10 mL) and extracted with EtOAc (2×10 mL). The combined organic layers were washed with brine and dried over Na₂SO₄. After filtration, the filtrate was concentrated under reduced pressure to afford 2′-chloro-5′-methoxy-6-methyl-N-(6-(4-oxocyclohexyl)thiazolo[4,5-b]pyrazin-2-yl)-[4,4′-bipyridine]-3-carboxamide (243 mg, 70% yield) as a yellow solid. LCMS (ESI, m/z): 509 [M+H]⁺.

Step 4: To a stirred solution of 2′-chloro-5′-methoxy-6-methyl-N-(6-(4-oxocyclohexyl)thiazolo[4,5-b]pyrazin-2-yl)-[4,4′-bipyridine]-3-carboxamide (185 mg, 0.4 mmol) in MeOH (5 mL) was slowly added NaBH₄ (28 mg, 0.7 mmol) in portions. The reaction mixture was stirred at 25° C. for 2 h. The mixture was quenched with water (50 mL) and extracted with DCM (2×100 mL). The combined organic layers were washed with brine and dried over Na₂SO₄. After filtration, the filtrate was concentrated under reduced pressure. The residue was purified by prep-HPLC (18-38% MeCN in water with 0.1% formic acid modifier) to afford 2′-chloro-N-(6-(4-hydroxycyclohexyl)thiazolo[4,5-b]pyrazin-2-yl)-5′-methoxy-6-methyl-[4,4′-bipyridine]-3-carboxamide as light yellow solid. LCMS (ESI, m/z): 511 [M+H]⁺. ¹H NMR (400 MHz, DMSO-d₆) δ 13.34 (s, 1H), 8.87 (s, 1H), 8.56 (s, 1H), 8.16 (s, 1H), 7.59 (s, 1H), 7.47 (s, 1H), 4.60 (d, J=4.2 Hz, 1H), 3.61 (s, 3H), 3.48 (s, 1H), 2.67 (s, 1H), 2.61 (s, 3H), 1.92 (m, 4H), 1.65 (m, 2H), 1.32 (d, J=13.1 Hz, 2H).

The cis/trans isomers were separated by prep-chiral-HPLC (CHIRAL ART Cellulose-SB column, 2×25 cm, 5 μm; 30%/70% MeOH/MTBE:Hex 1:1 with 0.1% TFA modifier; 20 mL/min flow rate; wave length: 218/230 nm) to afford two peaks:

Peak 1 RT=5.7 min (cis or trans)-2′-chloro-N-(6-(4-hydroxycyclohexyl)thiazolo[4,5-b]pyrazin-2-yl)-5′-methoxy-6-methyl-[4,4′-bipyridine]-3-carboxamide (56 mg, 32% yield) as a light yellow solid. LCMS (ESI, m/z): 511 [M+H]⁺. ¹H NMR (400 MHz, DMSO-d₆) δ 13.35 (s, 1H), 8.92 (s, 1H), 8.47 (s, 1H), 8.15 (s, 1H), 7.54 (s, 1H), 7.40 (s, 1H), 4.59 (d, J=4.4 Hz, 1H), 3.62 (s, 3H), 3.55-3.39 (m, 1H), 2.78 (s, 1H), 2.59 (s, 3H), 1.91-1.95 (m, 4H), 1.64-1.68 (m, 2H), 1.42-1.17 (m, 2H).

Peak 2 RT: 7.7 min (trans or cis)-2′-chloro-N-(6-(4-hydroxycyclohexyl)thiazolo[4,5-b]pyrazin-2-yl)-5′-methoxy-6-methyl-[4,4′-bipyridine]-3-carboxamide (2.2 mg, 1.2% yield) as a white solid. LCMS (ESI, m/z): 511 [M+H]⁺. ¹H NMR (400 MHz, DMSO-d₆) δ 13.3 (s, 1H), 8.90 (s, 1H), 8.51 (s, 1H), 8.16 (s, 1H), 7.57 (s, 1H), 7.45 (s, 1H), 4.38 (d, J=3.2 Hz, 1H), 3.90 (s, 1H), 3.62 (s, 3H), 2.87 (s, 1H), 2.60 (s, 3H), 1.96-2.06 (m, 2H), 1.76 (d, J=12.9 Hz, 2H), 1.24-1.35 (m, 4H).

### Example 446A & 446B: Synthesis of (cis or trans)-2′-chloro-N-(6-(4-(2-hydroxypropan-2-yl)cyclohexyl)thiazolo[4,5-b]pyrazin-2-yl)-5′-methoxy-6-methyl-[4,4′-bipyridine]-3-carboxamide (446A) & (trans or cis)-2′-chloro-N-(6-(4-(2-hydroxypropan-2-yl)cyclohexyl)thiazolo[4,5-b]pyrazin-2-yl)-5′-methoxy-6-methyl-[4,4′-bipyridine]-3-carboxamide (446B)

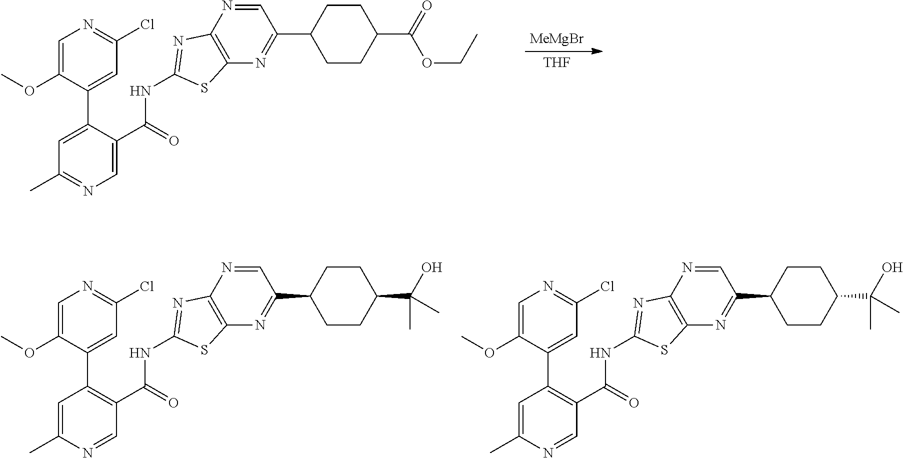

Into a 40 mL vial were added ethyl 4-(2-(2′-chloro-5′-methoxy-6-methyl-[4,4′-bipyridine]-3-carboxamido)thiazolo[4,5-b]pyrazin-6-yl)cyclohexane-1-carboxylate (210 mg, 3.70 mmol) and THF (2 mL) at room temperature. Methylmagnesium bromide (265 mg, 6.00 eq) was added dropwise at 0° C., and the resulting mixture was stirred for 1 h at 0° C. The reaction was quenched with water and extracted with EtOAc (3×100 mL). The resulting mixture was concentrated under reduced pressure. The residue was purified by Chiral-HPLC (CHIRALPAK IA column, 2×25 cm, 5 μm; 30% MeOH/70% MTBE:Hex 1:1 with 0.1% formic acid modifier; 20 mL/min flow rate; wave length: 218/326 nm) to afford two peaks:

Peak 1 RT=5.54 min (cis or trans)-2′-chloro-N-(6-(4-(2-hydroxypropan-2-yl)cyclohexyl)thiazolo[4,5-b]pyrazin-2-yl)-5′-methoxy-6-methyl-[4,4′-bipyridine]-3-carboxamide (25.4 mg, 12% yield) as a white solid. LCMS (ESI, m/z): 553 [M+H]⁺. ¹H NMR (400 MHz, DMSO-d₆) δ 13.28 (s, 1H), 8.88 (s, 1H), 8.65 (s, 1H), 8.17 (s, 1H), 7.59 (s, 1H), 7.47 (s, 1H), 3.92 (s, 1H), 3.63 (s, 3H), 3.26 (brs, 1H), 2.61 (s, 3H), 2.44-2.36 (m, 2H), 1.80-1.69 (m, 2H), 1.67-1.59 (m, 2H), 1.37-1.17 (m, 3H), 0.97 (s, 6H).

Peak 2 RT=12.14 min (trans or cis)-2′-chloro-N-(6-(4-(2-hydroxypropan-2-yl)cyclohexyl)thiazolo[4,5-b]pyrazin-2-yl)-5′-methoxy-6-methyl-[4,4′-bipyridine]-3-carboxamide (6.6 mg, 3.2% yield) as a white solid. LCMS (ESI, m/z): 553 [M+H]⁺. ¹H NMR (400 MHz, DMSO-d₆) δ 13.24 (s, 1H), 8.88 (s, 1H), 8.55 (s, 1H), 8.16 (s, 1H), 7.59 (s, 1H), 7.47 (s, 1H), 4.06 (s, 1H), 3.62 (s, 3H), 2.80 (t, J=12.3 Hz, 1H), 2.61 (s, 3H), 2.00-1.85 (m, 4H), 1.60-1.50 (m, 2H), 1.32-1.21 (m, 3H), 1.07 (s, 6H).

### Example 447A & 447B: Synthesis of (cis or trans)-2′-chloro-N-(6-(4-hydroxy-4-methylcyclohexyl)thiazolo[4,5-b]pyrazin-2-yl)-5′-methoxy-6-methyl-[4,4′-bipyridine]-3-carboxamide (447A) & (trans or cis)-2′-chloro-N-(6-(4-hydroxy-4-methylcyclohexyl)thiazolo[4,5-b]pyrazin-2-yl)-5′-methoxy-6-methyl-[4,4′-bipyridine]-3-carboxamide (447B)

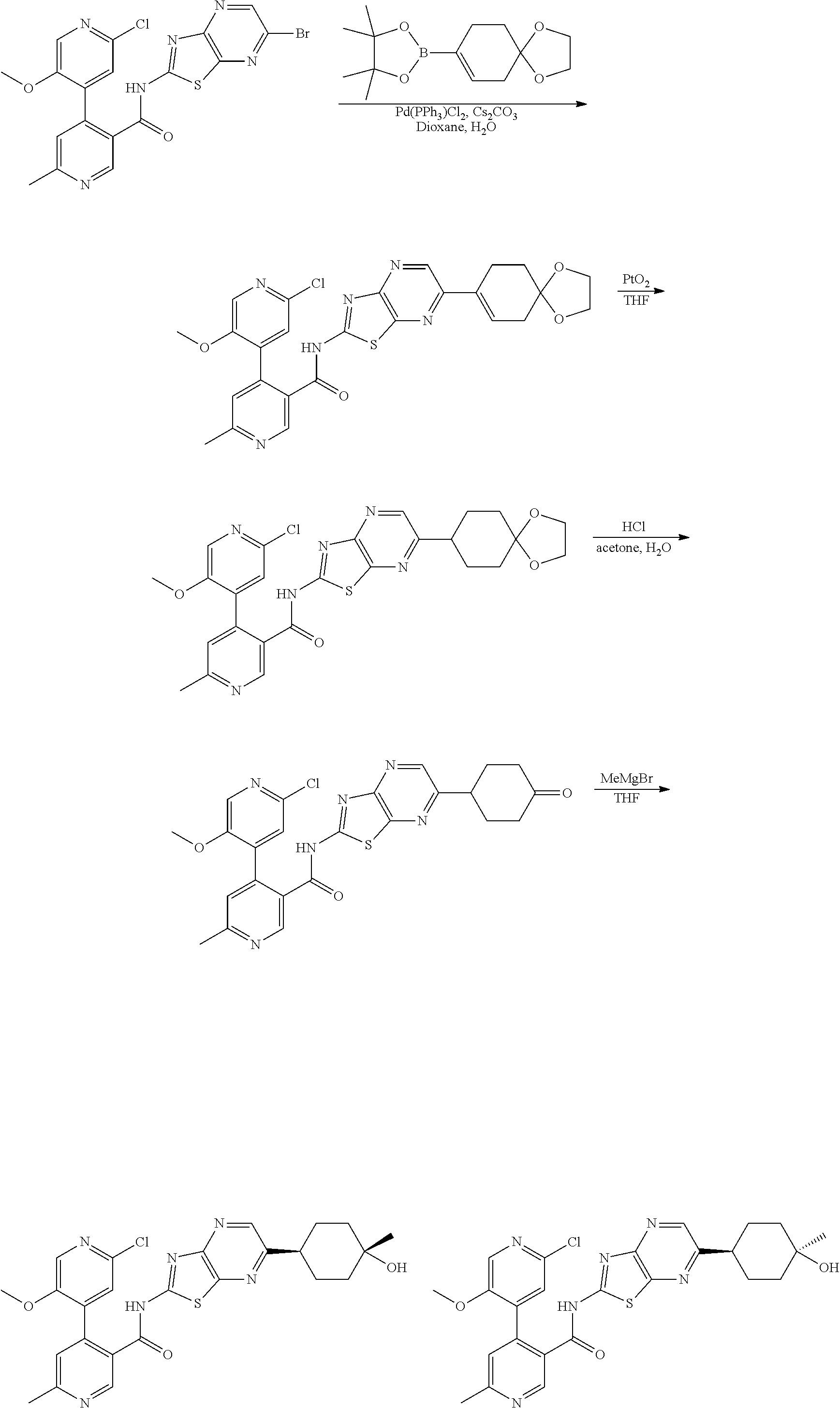

Step 1: To a solution of N-(6-bromothiazolo[4,5-b]pyrazin-2-yl)-2′-chloro-5′-methoxy-6-methyl-[4,4′-bipyridine]-3-carboxamide (2.0 g, 4.0 mmol) in 1,4-dioxane (30 mL) and water (3 mL) was added 4,4,5,5-tetramethyl-2-(1,4-dioxaspiro[4.5]dec-7-en-8-yl)-1,3,2-dioxaborolane (2.0 g, 6.0 mmol), PdCl₂(PPh₃)₂ (0.3 g, 0.4 mmol) and cesium carbonate (4.0 g, 12.0 mmol) in one portion at 25° C. The reaction mixture was charged with N₂ three times at 25° C. and the reaction was stirred at 90° C. for 16 h under a nitrogen atmosphere. The resulting mixture was cooled to 25° C. and concentrated under reduced pressure. The residue was purified by silica gel column chromatography, eluting with CH₂Cl₂/MeOH (10/1) to afford N-(6-(1,4-dioxaspiro[4.5]dec-7-en-8-yl)thiazolo[4,5-b]pyrazin-2-yl)-2′-chloro-5′-methoxy-6-methyl-[4,4′-bipyridine]-3-carboxamide (2.5 g, crude) as a yellow solid. LCMS (ESI, m/z): 551 [M+H]⁺.

Step 2: To a solution of N-(6-(1,4-dioxaspiro[4.5]dec-7-en-8-yl)thiazolo[4,5-b]pyrazin-2-yl)-2′-chloro-5′-methoxy-6-methyl-[4,4′-bipyridine]-3-carboxamide (2.5 g, 4.5 mmol) in THF (100 mL) was added PtO₂ (2.0 g, 9.0 mmol) in one portion at 25° C. The reaction system was replaced with H₂ (5.0 MPa) and the mixture was stirred at 50° C. for 16 h under a hydrogen atmosphere. The resulting mixture was cooled to 25° C., filtered, and concentrated under reduced pressure. The residue was purified by silica gel column chromatography, eluting with CH₂Cl₂/MeOH (1/1) to afford N-(6-(1,4-dioxaspiro[4.5]decan-8-yl)thiazolo[4,5-b]pyrazin-2-yl)-2′-chloro-5′-methoxy-6-methyl-[4,4′-bipyridine]-3-carboxamide (1.4 g, 58% yield) as a yellow solid. LCMS (ESI, m/z): 553 [M+H]⁺.

Step 3: To a solution of N-(6-(1,4-dioxaspiro[4.5]decan-8-yl)thiazolo[4,5-b]pyrazin-2-yl)-2′-chloro-5′-methoxy-6-methyl-[4,4′-bipyridine]-3-carboxamide (1.4 g, 2.5 mmol) in acetone (16 mL) was added HCl (92 mg, 2.5 mmol) in one portion at 25° C. The reaction mixture was stirred at 70° C. for 4 h. The resulting mixture was cooled to 25° C. and concentrated under reduced pressure. The residue was diluted with sat. NaHCO₃(aq.) (30 mL). The aqueous layer was extracted with DCM (3×50 mL) and the combined organic layers were dried over anhydrous Na₂SO₄. After filtration, the filtrate was concentrated under reduced pressure. The residue was purified by reverse-phase flash chromatography (10-50% MeCN in water with 0.1% formic acid modifier) to afford 2′-chloro-5′-methoxy-6-methyl-N-(6-(4-oxocyclohexyl)thiazolo[4,5-b]pyrazin-2-yl)-[4,4′-bipyridine]-3-carboxamide (643 mg, 50% yield) as a yellow solid. LCMS (ESI, m/z): 509 [M+H]⁺.

Step 4: Into a 30 mL sealed tube were added 2′-chloro-5′-methoxy-6-methyl-N-(6-(4-oxocyclohexyl)thiazolo[4,5-b]pyrazin-2-yl)-[4,4′-bipyridine]-3-carboxamide (100 mg, 0.2 mmol) in THF (20 mL) at 25° C. The reaction mixture was charged with N₂ three times at 25° C. and cooled to 0° C. To the above mixture was added MeMgBr (117 mg, 1.0 mmol) dropwise over 10 minutes at 0° C. The reaction mixture was stirred at 0° C. for 5 h under a nitrogen atmosphere. The reaction was quenched with sat. NH₄CO₃ (aq.) (5 mL) at 0° C. The resulting mixture was extracted with DCM (3×20 mL) and the combined organic layers were dried over anhydrous Na₂SO₄. After filtration, the filtrate was concentrated under reduced pressure. The residue was purified by reverse-phase flash chromatography (10-50% MeCN in water with 0.1% formic acid modifier) to afford the crude product. The crude product was purified by prep-HPLC (Sunfire prep C18 column, 30×150 mm, 5 μm; 48-65% MeOH in water with 0.1% formic acid modifier, 60 mL/min flow rate; wave length: 254 nm/220 nm) to afford two peaks:

Peak 1 (cis or trans)-2′-chloro-N-(6-(4-hydroxy-4-methylcyclohexyl)thiazolo[4,5-b]pyrazin-2-yl)-5′-methoxy-6-methyl-[4,4′-bipyridine]-3-carboxamide (9.3 mg, 9% yield) as a white solid. LCMS (ESI, m/z): 525 [M+H]⁺. ¹H NMR (400 MHz, DMSO-d₆) δ 13.34 (s, 1H), 8.88 (s, 1H), 8.59 (s, 1H), 8.16 (s, 1H), 7.59 (s, 1H), 7.47 (s, 1H), 4.37 (s, 1H), 3.62 (s, 3H), 2.86 (s, 1H), 2.61 (s, 3H), 1.85 (d, J=12.8 Hz, 2H), 1.68 (t, J=13.5 Hz, 4H), 1.60-1.47 (m, 2H), 1.19 (s, 3H).

Peak 2 (cis or trans)-2′-chloro-N-(6-(4-hydroxy-4-methylcyclohexyl)thiazolo[4,5-b]pyrazin-2-yl)-5′-methoxy-6-methyl-[4,4′-bipyridine]-3-carboxamide (14.7 mg, 14% yield) as a white solid. LCMS (ESI, m/z): 525 [M+H]⁺. ¹H NMR (400 MHz, DMSO-d₆) δ 13.35 (s, 1H), 8.88 (s, 1H), 8.54 (s, 1H), 8.17 (s, 1H), 7.60 (s, 1H), 7.47 (s, 1H), 4.08 (s, 1H), 3.62 (s, 3H), 2.79 (t, J=12.1 Hz, 1H), 2.61 (s, 3H), 2.07-1.90 (m, 2H), 1.67 (d, J=12.2 Hz, 4H), 1.52-1.38 (m, 2H), 1.16 (s, 3H).

TABLE BG
The following examples were prepared using a similar procedure to that described
for Example 447A & 447B

| Ex. | | Compound | LCMS | |
|---|---|---|---|---|
| No. | Structure | Name | [M + H]⁺ | NMR |
| 448^{ag} | 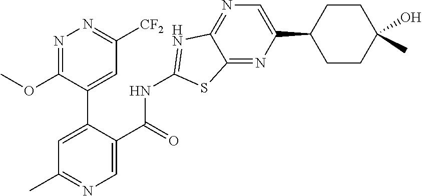 | (cis or trans)-4- (6-(difluoro- methyl)-3- methoxy- pyridazin-4-yl)- N-(6-(4- hydroxy-4- methylcyclo- hexyl)thiazolo [4,5-b]pyrazin- 2-yl)-6-methyl- nicotinamide | 542 | ¹H NMR (400 MHz, DMSO- d₆) δ 13.51 (s, 1H), 9.06 (s, 1H), 8.46 (s, 1H), 8.02 (s, 1H), 7.48 (s, 1H), 7.26 (t, J = 54.2 Hz, 1H), 4.37 (s, 1H), 3.84 (s, 3H), 2.86-2.76 (m, 1H), 2.61 (s, 3H), 1.84-1.80 (m, 2H), 1.70-1.60 (m, 4H), 1.58-1.49 (m, 2H), 1.90 (s, 3H) |
| 449^{ag} | 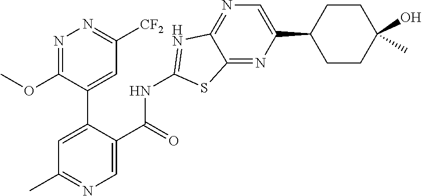 | (trans or cis)-4- (6-(difluoro- methyl)-3- methoxy- pyridazin-4-yl)- N-(6-(4- hydroxy-4- methylcyclo- hexyl)thiazolo [4,5-b]pyrazin- 2-yl)-6-methyl- nicotinamide | 542 | ¹H NMR (400 MHz, DMSO- d₆) δ 13.53 (s, 1H), 9.04 (s, 1H), 8.46 (s, 1H), 8.05 (s, 1H), 7.51 (s, 1H), 7.26 (t, J = 54.2 Hz, 1H), 4.08 (s, 1H), 3.85 (s, 3H), 2.80-2.70 (m, 1H), 2.62 (s, 3H), 1.98-1.95 (m, 2H), 1.67-1.62 (m, 4H), 1.46-1.40 (m, 2H), 1.15 (s, 3H) |

^{ag}Chiral separation conditions for Example 448 and 449: Prep-HPLC Xselect CSH C18 OBD Column 30 × 150 mm, 5 μm; 28-45% MeCN in water with
0.1% formic acid modifier; 60 mL/min flow rate; wave length: 254 nm/220 nm nm to give two peaks: Peak 1 RT: 7.52 min (crude Example 448), Peak
2 RT: 9.18 min (crude Example 449). Peak 1 (crude Example 448) was repurified by prep-HPLC YMC-Actus Triart C18 EXRS column 30 × 150 mm,
5 μm; 39-59% MeOH in Water with 10 mmol/L NH₄HCO₃ modifier; 60 mL/min flow rate; wave length: 254 nm/220 nm nm; RT 8.18 min (Example
448). Peak 2 (crude Example 449) was repurified by prep-HPLC XBridge Prep OBD C18 Column, 30 × 150 mm, 10 μm; 35-55% MeOH in Water with
10 mmol/L NH4CO3 and 0.05% NH3H2O modifier; 60 mL/min flow rate; wave length: 254 nm/220 nm nm; RT 9.43 (Example 449)

### Example 450: Synthesis of 2′-chloro-N-(6-(4-hydroxytetrahydro-2H-pyran-4-yl)thiazolo[4,5-b]pyrazin-2-yl)-5′-methoxy-6-methyl-[4,4′-bipyridine]-3-carboxamide

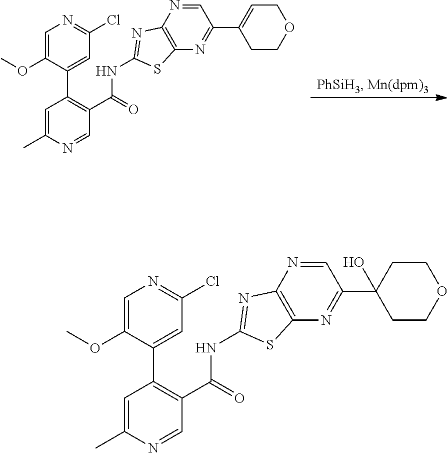 [SMILES: COc1cnc(Cl)cc1-c1cc(C)ncc1C(=O)Nc1nc2ncc(C3=CCOCC3)nc2s1 ; "PhSiH3, Mn(dpm)3" ; COc1cnc(Cl)cc1-c1cc(C)ncc1C(=O)Nc1nc2ncc(C3(O)CCOCC3)nc2s1]

A mixture of 2′-chloro-N-(6-(3,6-dihydro-2H-pyran-4-yl)thiazolo[4,5-b]pyrazin-2-yl)-5′-methoxy-6-methyl-[4,4′-bipyridine]-3-carboxamide (200 mg, 0.40 mmol), phenylsilane (131 mg, 1.21 mmol) and Mn(dpm)₃ (24 mg, 0.04 mmol) in isopropanol (4.5 mL) and CH₂Cl₂ (0.5 mL) was stirred for 20 h at 25° C. The resulting mixture was concentrated under reduced pressure. The residue was purified by silica gel column chromatography, eluting with CH₂Cl₂/MeOH (0-10%) to afford crude product. The material was further purified by reverse phase chromatography (47-62% MeOH in water with 0.1% formic acid modifier) to afford 2′-chloro-N-(6-(4-hydroxytetrahydro-2H-pyran-4-yl)thiazolo[4,5-b]pyrazin-2-yl)-5′-methoxy-6-methyl-[4,4′-bipyridine]-3-carboxamide (66 mg, 32% yield) as off-white solid. LCMS (ESI, m/z): 513 [M+H]⁺. ¹H NMR (400 MHz, DMSO-d₆) δ 13.41 (s, 1H), 8.89 (d, J=6.3 Hz, 2H), 8.17 (s, 1H), 7.61 (s, 1H), 7.49 (s, 1H), 5.60 (s, 1H), 3.84-3.71 (m, 4H), 3.62 (s, 3H), 2.61 (s, 3H), 2.24-2.12 (m, 2H), 1.64 (dd, J=13.7, 2.2 Hz, 2H).

### Example 451A & 451B: Synthesis of (S or R)-2′-chloro-5′-methoxy-6-methyl-N-(6-(1-(2,2,2-trifluoroethyl)pyrrolidin-3-yl)thiazolo[4,5-b]pyrazin-2-yl)-[4,4′-bipyridine]-3-carboxamide (451A) & (R or S)-2′-chloro-5′-methoxy-6-methyl-N-(6-(1-(2,2,2-trifluoroethyl)pyrrolidin-3-yl)thiazolo[4,5-b]pyrazin-2-yl)-[4,4′-bipyridine]-3-carboxamide (451B)

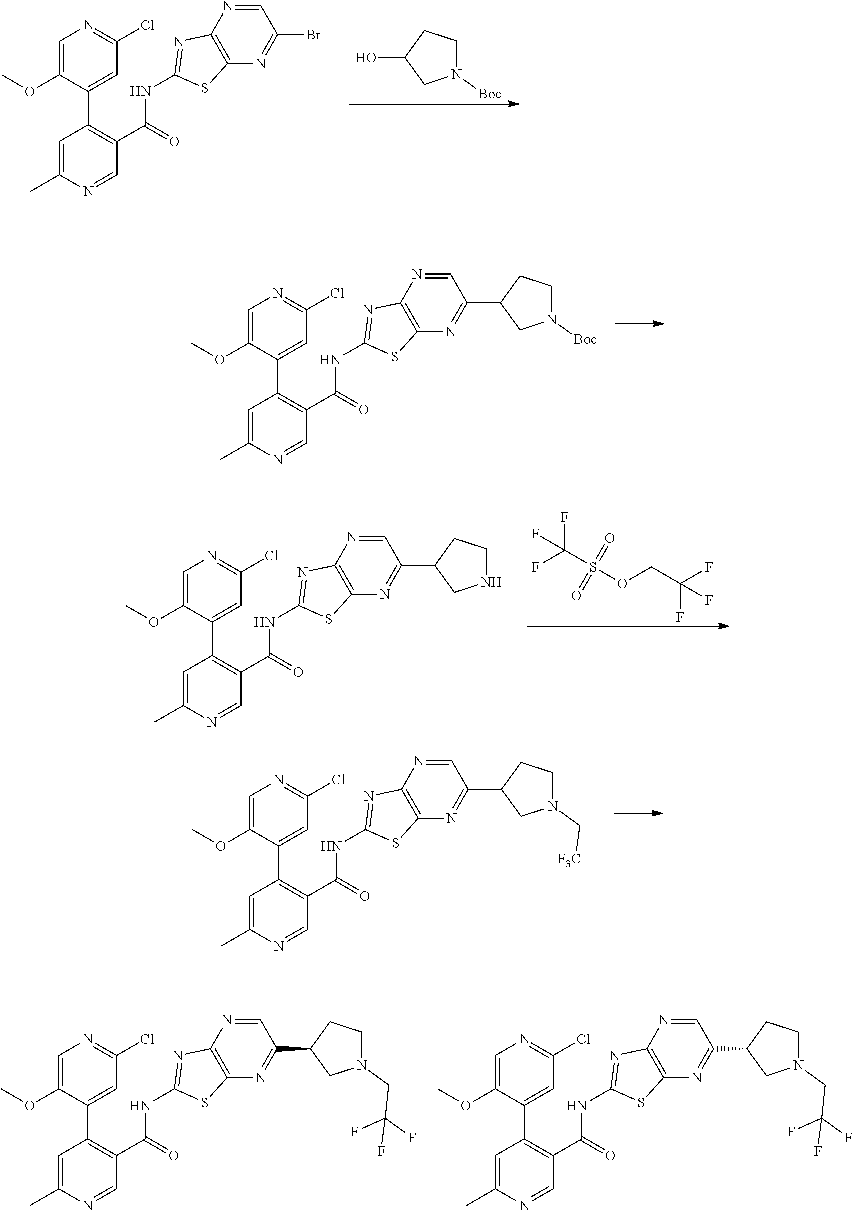

Step 1: A mixture of tert-butyl 3-hydroxypyrrolidine-1-carboxylate (366 mg, 1.95 mmol) and 5,7-di-tert-butyl-3-phenylbenzo[d]oxazol-3-ium tetrafluoroborate (707 mg, 1.79 mmol) in MTBE (5 mL) was stirred for 30 min at 25° C. under a nitrogen atmosphere. To the above mixture was added pyridine (142 mg, 1.79 mmol) and the resulting mixture was stirred for an additional 10 min at 25° C. To the above mixture were added N-(6-bromothiazolo[4,5-b]pyrazin-2-yl)-2′-chloro-5′-methoxy-6-methyl-[4,4′-bipyridine]-3-carboxamide (550 mg, 1.12 mmol) in DMA (5 mL), [4,4′-bis(tert-butyl)-2,2′-bipyridine]nickel dibromide (54 mg, 0.11 mmol), [Ir(dtbbpy)(ppy)₂]PF₆ (51 mg, 0.04 mmol), and quinuclidine (217 mg, 1.95 mmol). The resulting mixture was stirred for an additional 16 h at 25° C. under blue light (450 nm LED modules at 100% light intensity). The mixture was diluted with water (20 mL) and extracted with EtOAc (3×50 mL). The combined organic layers were washed with brine (2×20 mL) and dried over anhydrous Na₂SO₄. After filtration, the filtrate was concentrated under reduced pressure. The residue was purified by reverse-phase flash chromatography (10-50% MeCN in water with 0.1% formic acid modifier) to afford tert-butyl 3-(2-(2′-chloro-5′-methoxy-6-methyl-[4,4′-bipyridine]-3-carboxamido)thiazolo[4,5-b]pyrazin-6-yl)pyrrolidine-1-carboxylate (300 mg, 46% yield) as a yellow solid. LCMS (ESI, m/z): 582 [M+H]⁺.

Step 2: To a stirred solution of tert-butyl 3-(2-(2′-chloro-5′-methoxy-6-methyl-[4,4′-bipyridine]-3-carboxamido)thiazolo[4,5-b]pyrazin-6-yl)pyrrolidine-1-carboxylate (300 mg, 0.51 mmol) in CH₂Cl₂ (3 mL) was added HCl in 1,4-dioxane (1 mL, 4 M) at room temperature. The reaction mixture was stirred for 2 h at 25° C. under a nitrogen atmosphere. The resulting mixture was concentrated under reduced pressure to afford 2′-chloro-5′-methoxy-6-methyl-N-(6-(pyrrolidin-3-yl)thiazolo[4,5-b]pyrazin-2-yl)-[4,4′-bipyridine]-3-carboxamide (220 mg, 89% yield) as white solid. LCMS (ESI, m/z): 482 [M+H]⁺.

Step 3: To a stirred solution of 2′-chloro-5′-methoxy-6-methyl-N-(6-(pyrrolidin-3-yl)thiazolo[4,5-b]pyrazin-2-yl)-[4,4′-bipyridine]-3-carboxamide (200 mg, 0.41 mmol) in THF (5 mL) were added 2,2,2-trifluoroethyl trifluoromethanesulfonate (101 mg, 0.43 mmol) and TEA (84 mg, 0.83 mmol) at room temperature. The resulting mixture was stirred for 16 h at 80° C. under a nitrogen atmosphere. The mixture was cooled to room temperature, filtered, and the filtrate was concentrated under reduced pressure. The residue was purified by reverse-phase flash chromatography (10-50% MeCN in water with 0.1% formic acid modifier) to afford racemic 2′-chloro-5′-methoxy-6-methyl-N-(6-(1-(2,2,2-trifluoroethyl)pyrrolidin-3-yl)thiazolo[4,5-b]pyrazin-2-yl)-[4,4′-bipyridine]-3-carboxamide (89 mg) as a yellow solid. The racemate was purified by prep-chiral HPLC (Lux 5 um Cellulose-4 column 3×25 cm, 5 μm; 50%/50% EtOH/hexane with 0.1% formic acid modifier; 40 mL/min flow rate; wave length: 218/328 nm) to afford two peaks:

Peak 1 RT=10.325 min: (S or R)-2′-chloro-5′-methoxy-6-methyl-N-(6-(1-(2,2,2-trifluoroethyl)pyrrolidin-3-yl)thiazolo[4,5-b]pyrazin-2-yl)-[4,4′-bipyridine]-3-carboxamide (33 mg, 38%) as a yellow solid. LCMS (ESI, m/z): 564 [M+H]⁺. ¹H NMR (400 MHz, DMSO-d₆) δ 13.32 (s, 1H), 8.89 (s, 1H), 8.59 (s, 1H), 8.16 (s, 1H), 7.59 (s, 1H), 7.47 (s, 1H), 3.74-3.59 (m, 4H), 3.45-3.35 (m, 2H), 3.23 (t, J=8.4 Hz, 1H), 3.03-2.85 (m, 3H), 2.61 (s, 3H), 2.34-2.21 (m, 1H), 2.14-2.02 (m, 1H).

Peak 2 RT=16.775 min: (R or S)-2′-chloro-5′-methoxy-6-methyl-N-(6-(1-(2,2,2-trifluoroethyl)pyrrolidin-3-yl)thiazolo[4,5-b]pyrazin-2-yl)-[4,4′-bipyridine]-3-carboxamide (30 mg, 36%) as a yellow solid. LCMS (ESI, m/z): 564 [M+H]⁺. ¹H NMR (400 MHz, DMSO-d₆) δ 13.35 (s, 1H), 8.89 (s, 1H), 8.59 (s, 1H), 8.17 (s, 1H), 7.60 (s, 1H), 7.48 (s, 1H), 3.70 (q, J=8.1 Hz, 1H), 3.62 (s, 3H), 3.46-3.39 (m, 1H), 3.39-3.36 (m, 1H), 3.23 (t, J=8.4 Hz, 1H), 2.99 (q, J=7.7 Hz, 1H), 2.93-2.85 (m, 2H), 2.61 (s, 3H), 2.34-2.21 (m, 1H), 2.14-2.02 (m, 1H).

### Example 291. Biological Activity of the Compounds of the Present Disclosure

The biological activity of the compounds of the present disclosure was determined utilizing the assay described herein.

### ATPase Assay

POLΘ-mediated ATP hydrolysis was measured using the ADP-Glo Kinase Assay (Promega Corporation, Madison, WI). Reactions were performed in white 384-well small-volume microplates (Greiner Bio-One, Frickenhausen, Germany) using a total volume of 15 μL. The ATPase reaction consisted of 1 nM biotinylated Avi-POLΘ[2-894], 5 nM of a 50 nucleotide poly-thymine repeat single-stranded DNA (PolyT(50) ssDNA), and 100 μM ATP in 5 μL of assay buffer (50 mM Tris, pH 7.5, 80 mM KCl, 10 MgCl₂, 1 mM DTT, 5% glycerol, 0.01% BSA, 0.01% Tween-20). Inhibition of POLO ATPase activity was determined by preparing 11-point serial dilutions of the test compounds in 100% DMSO, then transferring 30 nL of the compounds into the ATPase reaction using an Echo acoustic liquid handler (Beckman Coulter, Brea, CA). DMSO-treated wells containing either all of the reaction components (1 nM biotinylated Avi-POLΘ[2-894], 5 nM PolyT(50) ssDNA, 100 μM ATP) or with assay buffer substituted for the biotinylated Avi-POLΘ[2-894](0 nM biotinylated Avi-POLΘ[2-894], 5 nM PolyT(50) ssDNA, 100 μM ATP) were used to define the maximum and minimum response in the assay. Following a one-hour incubation at ambient temperature, 5 μL of ADP-Glo Reagent was added to the ATPase reaction, and this mixture was allowed to incubate at ambient temperature for an additional hour. Lastly, 5 μL of Kinase Detection Reagent was added, followed by a third one-hour incubation at ambient temperature. The reagent concentrations listed represent the amounts only in the 5 μL ATPase reaction volume. Luminescence signal was measured using the EnVision multimode plate reader (Perkin Elmer, Waltham, MA). To determine the inhibitory potency (AC₅₀) of the test compounds, the luminescence signal was normalized to percent activity using the median value of the maximum signal wells (corresponding to 0% activity) and the median value of the minimum signal wells (corresponding to −100% activity). Non-linear regression analysis using the four-parameter logistic equation was conducted on the percent activity values of the compound dose-responses, yielding the AC₅₀ value for each compound. Genedata Screener (Genedata, Basel, Switzerland) was used for the data analysis.

### DLD1 Viability Assay

Cell lines DLD1 WT and DLD1 BRCA2 (−/−) (Horizon Discovery Cat No. HD PAR-008 and HD-105-007) were plated in 384-well culture plates at seeding densities of 100 cells/50 ul/well and 200 cells/50 uL/well respectively. The seeding densities are pre-determined from the growth curve kinetics performed to ensure log linear phase of cell growth for these 2 cell lines. These assay plates are then placed in the incubator at 37° C., 5% CO₂, relative humidity>90% for a few hours before addition of test compounds. Compounds are dispensed using the Tecan HPD300 or Echo based on the plate map in the appropriate wells of the 384-well plates. The cells are treated with nine-point dose response with 3-fold serial dilutions of test compounds starting at 10 uM top concentration. After addition of the test compounds, the plates are incubated at 37° C., 5% CO₂, relative humidity>90% for 7 days. CellTiter-Glo (Promega Cat. No. G7573), ATP detection reagent is prepared according to manufacturer's specifications. Cell assay plates are removed from incubator and bring to room temperature (RT) by letting them sit at bench top for at least 15 min. Using the MultiDrop liquid handler or a multichannel pipette, 35 μL/well of CTG is added and the plates are then placed shakes for 5 seconds, RT. These plates are then incubated for 30 minutes in the dark at RT before measuring luminescence using Spectramax/Enspire/Ensight/Envision/PheraSTAR. Data is analyzed using the DMSO control wells as the 0% inhibition control (high signal) and the 10 μM Doxorubicin control wells as the 100% inhibition control (low signal) control to calculate percent inhibition and IC50s for the test compound dose responses.

The antagonist activity values IC₅₀ of each compound is shown in Table AH below.

Values of Biochemical ATPase ADPGlo IC₅₀ in Table AH are presented in ranges, in which 0.001 μM≤“+”<0.015 μM, 0.015 μM<“++”≤0.050 μM, 0.050 μM≤“+++”≤0.150 μM, 0.150 μM≤“++++”≤0.900 μM, and 0.900 μM<“+++++.”

Values of Viability IC₅₀ (μM) DLD1 BRCA2 null in Table AH are presented in ranges, in which 0.001 μM<“*”<0.100 μM, 0.100 μM<“**”<0.250 μM, 0.250 μM<“***”<0.500 μM, 0.500 μM<“***”<2.000 μM, and 2.000 μM<

Values of Viability IC₅₀ (μM) DLD1 WT in Table AH are presented in ranges, in which 0.001≤M “#”<2.500 μM, 2.500+M “##”<5.000 μM, 5.000 μM<“###” 7.500 μM, 7.500 μM≤“####”<10.00 μM, and 10.00 μM≤“####.”

TABLE AH

| | | | Viability | |
|---|---|---|---|---|
| | | | IC50 | |
| | | Biochemical | (μM) | |
| | | ATPase | DLD1 | Viability |
| Compound | | ADPGlo | BRCA2 | IC50 (μM) |
| No. | Example No. | IC50 (μM) | null | DLD1 WT |
| 333 | 1 | + | **** | ##### |
| 330 | 2 | ++ | ***** | ##### |
| 332 | 3 | ++ | ***** | ##### |
| 316 | 4 | ++ | | |
| 329 | 5 | +++ | | |
| 331 | 6 | +++ | | |
| 301 | 7 | + | ***** | ##### |
| 274 | 8 | + | *** | ##### |
| 299 | 9 | + | | |
| 280 | 10 | + | **** | ##### |
| 275 | 11 | + | **** | ##### |
| 285 | 12 | + | **** | ##### |
| 278 | 13 | + | **** | ##### |
| 292 | 14 | + | | |
| 312 | 15 | + | ***** | ##### |
| 305 | 16 | + | ***** | ##### |
| 313 | 17 | + | ***** | ##### |
| 307 | 18 | + | **** | ##### |
| 295 | 19 | + | ***** | ##### |
| 294 | 20 | + | ***** | ##### |
| 298 | 21 | + | **** | ##### |
| 293 | 22 | + | ***** | ##### |
| 306 | 23 | + | ***** | ##### |
| 281 | 24 | + | ***** | ##### |
| 297 | 25 | ++ | | |
| 300 | 26 | + | ***** | ##### |
| 276 | 27 | + | ** | ##### |
| 277 | 28 | ++ | | |
| 309 | 29 | + | **** | ##### |
| 311 | 30 | + | ***** | ##### |
| 310 | 31 | + | ***** | ##### |
| 282 | 32 | + | * | ##### |
| 272 | 33 | + | | |
| 268 | 34 | + | ***** | ##### |
| 308 | 35 | + | ***** | ##### |
| 267 | 36 | ++ | | |
| 302 | 37 | + | *** | ##### |
| 163 | 38 | ++++ | ***** | ##### |
| 100 | 39 | ++ | ***** | ##### |
| 96 | 40 | ++ | **** | ### |
| 137 | 41 | ++++ | | |
| 109 | 42 | +++ | ***** | ##### |
| 85 | 43 | + | *** | #### |
| 167 | 44 | ++++ | | |
| 118 | 45 | +++ | ***** | #### |
| 138 | 46 | ++++ | ***** | ##### |
| 132 | 47 | +++ | **** | ##### |
| 134 | 48 | +++ | | |
| 131 | 49 | +++ | ***** | ### |
| 104 | 50 | ++ | **** | ##### |
| 161 | 51 | ++++ | | |
| 111 | 52 | ++ | ***** | ##### |
| 110 | 53 | ++ | | |
| 186 | 54 | +++++ | | |
| 98 | 55 | ++ | **** | ##### |
| 75 | 56 | + | ** | ### |
| 95 | 58 | + | *** | ### |
| 117 | 59 | ++ | | |
| 126 | 60 | +++ | **** | ##### |
| 82 | 61 | + | ** | ##### |
| 72 | 62 | + | * | ##### |
| 83 | 63 | + | **** | ##### |
| 87 | 64 | + | *** | ##### |
| 80 | 65 | + | *** | ##### |
| 76 | 66 | + | *** | ##### |
| 62 | 67 | + | ** | ##### |
| 91 | 68 | + | **** | #### |
| 154 | 69 | ++++ | | |
| 174 | 70 | +++++ | | |
| 162 | 71 | ++++ | | |
| 172 | 72 | +++++ | | |
| 194 | 73 | +++++ | | |
| 145 | 74 | ++++ | | |
| 197 | 75 | +++++ | | |
| 135 | 76 | ++++ | | |
| 155 | 77 | ++++ | | |
| 142 | 78 | ++++ | | |
| 195 | 79 | +++++ | | |
| 196 | 80 | +++++ | | |
| 61 | 81 | + | * | ##### |
| 64 | 82 | + | * | ##### |
| 63 | 83 | + | *** | ##### |
| 66 | 84 | + | * | ##### |
| 23 | 85 | + | * | ##### |
| 156 | 86 | ++++ | | |
| 121 | 87 | +++ | **** | # |
| 144 | 88 | ++++ | ***** | ##### |
| 140 | 89 | ++++ | ***** | ##### |
| 179 | 90 | +++++ | | |
| 157 | 91 | ++++ | | |
| 164 | 92 | ++++ | | |
| 176 | 93 | +++++ | | |
| 150 | 94 | ++++ | | |
| 188 | 95 | +++++ | | |
| 177 | 96 | +++++ | | |
| 77 | 97 | + | **** | ##### |
| 107 | 98 | ++ | **** | ##### |
| 84 | 99 | + | | |
| 60 | 100 | + | * | ## |
| 69 | 101 | + | * | ##### |
| 130 | 102 | +++ | | |
| 93 | 103 | + | | |
| 101 | 104 | ++ | | |
| 94 | 105 | + | *** | ##### |
| 106 | 106 | ++ | | |
| 115 | 107 | +++ | | |
| 79 | 108 | + | ***** | ##### |
| 70 | 109 | + | *** | ## |
| 148 | 110 | ++++ | | |
| 103 | 111 | ++ | | |
| 180 | 112 | +++++ | | |
| 133 | 113 | +++ | | |
| 58 | 114 | ++ | | |
| 120 | 115 | +++ | ***** | ##### |
| 92 | 116 | + | **** | ##### |
| 122 | 117 | +++ | | |
| 116 | 118 | ++ | | |
| 105 | 119 | ++ | | |
| 114 | 120 | +++ | | |
| 89 | 121 | + | * | # |
| 152 | 122 | ++++ | | |
| 128 | 123 | +++ | **** | #### |
| 159 | 124 | ++++ | | |
| 146 | 125 | ++++ | | |
| 143 | 126 | ++++ | | |
| 189 | 127 | +++++ | | |
| 185 | 128 | +++++ | | |
| 183 | 129 | +++++ | | |
| 181 | 130 | +++++ | | |
| 187 | 131 | +++++ | | |
| 190 | 132 | +++++ | | |
| 191 | 133 | +++++ | | |
| 160 | 134 | ++++ | | |
| 192 | 135 | +++++ | | |
| 193 | 136 | +++++ | | |
| 201 | 137 | +++++ | | |
| 169 | 138 | ++++ | | |
| 141 | 139 | ++++ | | |
| 166 | 140 | ++++ | | |
| 200 | 141 | +++++ | | |
| 168 | 142 | ++++ | | |
| 173 | 143 | +++++ | | |
| 184 | 144 | +++++ | | |
| 123 | 145 | +++ | | |
| 97 | 146 | ++ | | |
| 127 | 147 | +++ | | |
| 119 | 148 | +++ | ***** | ##### |
| 113 | 149 | +++ | | |
| 153 | 150 | ++++ | | |
| 124 | 151 | +++ | | |
| 158 | 152 | ++++ | | |
| 151 | 153 | ++++ | | |
| 171 | 154 | +++++ | | |
| 112 | 155 | +++ | | |
| 178 | 156 | +++++ | | |
| 170 | 157 | ++++ | | |
| 354 | 158 | ++++ | | |
| 351 | 159 | ++++ | | |
| 182 | 160 | +++++ | | |
| 356 | 161 | +++++ | | |
| 27 | 162 | ++ | | |
| 149 | 163 | ++++ | | |
| 165 | 164 | ++++ | | |
| 88 | 165 | + | **** | ##### |
| 346 | 166 | +++ | | |
| 345 | 167 | +++ | | |
| 336 | 168 | ++ | ***** | ##### |
| 320 | 169 | + | | |
| 318 | 170 | + | | |
| 317 | 171 | + | | |
| 353 | 172 | ++++ | | |
| 342 | 173 | +++ | | |
| 344 | 174 | +++ | | |
| 349 | 175 | ++++ | | |
| 352 | 176 | ++++ | | |
| 340 | 177 | ++ | ***** | ##### |
| 315 | 178 | + | | |
| 314 | 179 | + | | |
| 279 | 180 | + | ***** | ##### |
| 286 | 181 | ++++ | | |
| 296 | 182 | + | | |
| 303 | 183 | +++++ | | |
| 283 | 184 | + | ***** | ##### |
| 357 | 185 | +++++ | | |
| 347 | 186 | +++ | | |
| 341 | 187 | ++ | | |
| 325 | 188 | ++++ | | |
| 328 | 189 | ++ | | |
| 326 | 190 | + | ***** | ##### |
| 327 | 191 | ++ | | |
| 321 | 192 | +++ | | |
| 350 | 193 | ++++ | | |
| 338 | 194 | ++ | | |
| 343 | 195 | +++ | | |
| 334 | 196 | + | ***** | ##### |
| 324 | 197 | ++ | ***** | ##### |
| 339 | 198 | ++ | | |
| 266 | 199 | ++ | | |
| 348 | 200 | ++++ | | |
| 202 | 201 | +++++ | | |
| 199 | 202 | +++++ | | |
| 175 | 203 | +++++ | | |
| 125 | 204 | +++ | | |
| 136 | 205 | ++++ | | |
| 68 | 206 | + | ** | ##### |
| 49 | 207 | + | ** | ##### |
| 78 | 208 | + | **** | ##### |
| 38 | 209 | ++ | | |
| 31 | 210A | ++ | | |
| 30 | 210B | + | ** | ##### |
| 54 | 211 | ++ | | |
| 39 | 212 | ++ | | |
| 53 | 213 | + | | |
| 337 | 214 | ++ | ***** | ##### |
| 335 | 215 | + | ***** | ##### |
| 304 | 216 | ++++ | | |
| 55 | 217 | +++ | | |
| 86 | 218 | + | | |
| 37 | 219 | + | **** | ##### |
| 48 | 220 | + | **** | ##### |
| 47 | 221 | + | ***** | ##### |
| 44 | 222 | ++ | | |
| 33 | 223 | + | ***** | ##### |
| 43 | 224 | + | ***** | ##### |
| 42 | 225 | + | *** | ##### |
| 26 | 226 | + | | |
| 41 | 227 | + | | |
| 40 | 228 | + | | |
| 36 | 229 | ++ | | |
| 19 | 230 | ++++ | | |
| 28 | 231 | + | | |
| 34 | 232 | + | | |
| 20 | 233 | + | *** | ##### |
| 29 | 234 | + | **** | ##### |
| 21 | 235 | + | **** | ##### |
| 18 | 236 | + | **** | ##### |
| 10 | 237 | + | * | ##### |
| 35 | 238 | ++ | | |
| 17 | 239 | + | **** | ##### |
| 289 | 240 | + | ***** | ##### |
| 287 | 241 | + | **** | ##### |
| 291 | 242 | + | ***** | ##### |
| 288 | 243 | + | *** | ##### |
| 290 | 244 | + | ***** | ##### |
| 5 | 245 | + | | |
| 11 | 246 | + | ** | ##### |
| 3 | 247 | + | * | ##### |
| 12 | 248 | + | * | ##### |
| 2 | 249 | + | * | ##### |
| 56 | 250 | + | ***** | ##### |
| 24 | 251 | + | * | ##### |
| 147 | 252 | ++++ | | |
| 108 | 253 | ++ | | |
| 57 | 254 | +++ | | |
| 52 | 255 | +++ | | |
| 358 | 256 | +++++ | | |
| 22 | 257 | + | ***** | ##### |
| 16 | 258 | ++++ | | |
| 51 | 259A | + | ***** | ##### |
| 50 | 259B | + | ***** | ##### |
| 46 | 260 | + | | |
| 74 | 261 | + | * | ##### |
| 25 | 262 | + | *** | ##### |
| 99 | 263 | ++ | | |
| 65 | 264 | + | ** | ##### |
| 71 | 265 | + | **** | ##### |
| 59 | 266 | + | * | ##### |
| 102 | 267 | ++ | | |
| 45 | 268 | + | *** | ##### |
| 8 | 269A | + | | |
| 9 | 269B | + | | |
| 7 | 270 | + | | |
| 1 | 271 | + | * | ##### |
| 15 | 272 | + | ** | ##### |
| 13 | 273 | + | * | ##### |
| 129 | 274 | +++ | | |
| 81 | 275 | + | ***** | ##### |
| 139 | 276 | ++++ | | |
| 90 | 277 | + | ***** | ##### |
| 67 | 278 | + | ** | ##### |
| 73 | 279 | + | *** | ##### |
| 14 | 280 | + | **** | ##### |
| 284 | 281 | + | | |
| 323 | 282 | ++ | | |
| 319 | 283 | +++ | | |
| 322 | 284 | ++ | | |
| 271 | 285 | + | | |
| 273 | 286 | + | | |
| 269 | 287 | ++ | | |
| 270 | 288 | ++ | | |
| 6 | 289 | + | | |
| 4 | 290 | + | * | ##### |
| 32 | Intermediate 20 | +++++ | | |
| 515 | 292A | + | ** | ##### |
| 516 | 292B | + | | |
| 513 | 293 | ++ | | |
| 517 | 294 | +++++ | | |
| 445 | 295 | | * | ##### |
| 448 | 296 | | * | ##### |
| 446 | 297 | | * | ##### |
| 512 | 298 | + | * | ##### |
| 510 | 299 | + | ** | ##### |
| 479 | 300 | + | | |
| 473 | 301 | ++ | | |
| 443 | 302 | | * | ##### |
| 435 | 303 | | * | ##### |
| 431 | 304 | | * | ##### |
| 434A | 306 | | * | ##### |
| 454 | 427 | + | * | ##### |
| 514 | 307 | + | * | ##### |
| 499 | 308A | + | ** | ##### |
| 498 | 308B | + | * | ##### |
| 496 | 309 | + | | |
| 497 | 310 | + | * | ##### |
| 421 | 311 | | * | ##### |
| 411 | 312 | | * | ##### |
| 417 | 313 | + | | |
| 449 | 314 | | * | ##### |
| 493 | 315 | +++ | | |
| 495 | 316A | ++++ | | |
| 494 | 316B | ++ | | |
| 451A | 317 | + | * | ##### |
| 518 | 318 | + | * | ##### |
| 519 | 319 | + | * | ##### |
| 520 | 320 | + | ***** | ##### |
| 384 | 321 | | ** | ##### |
| 521 | 322 | + | * | ##### |
| 522 | 323 | + | * | ##### |
| 372 | 324 | ++ | | |
| 368 | 325 | + | * | ##### |
| 500 | 326 | ++ | | |
| 501 | 327 | + | | |
| 509 | 328 | + | * | ##### |
| 502 | 329 | + | * | ##### |
| 511 | 330 | + | * | ## |
| 504 | 331 | + | * | ##### |
| 506 | 332 | + | * | ##### |
| 491 | 333 | | * | ##### |
| 483 | 334 | | ** | ##### |
| 488 | 335 | | * | ##### |
| 490 | 336 | | * | ##### |
| 481 | 337 | | *** | #### |
| 485 | 338 | | ** | ##### |
| 470 | 339 | + | | |
| 463 | 340 | | * | ##### |
| 489 | 341 | | * | ##### |
| 471 | 342 | | * | ##### |
| 480 | 343 | | * | ##### |
| 482 | 344 | | * | ##### |
| 486 | 345 | | * | ##### |
| 472 | 346 | | * | ### |
| 466 | 347 | | * | #### |
| 474 | 348 | | * | #### |
| 475 | 349 | | * | ##### |
| 467 | 350 | | * | ##### |
| 468 | 351 | | * | ### |
| 469 | 352 | + | | |
| 462 | 353 | | * | ##### |
| 464 | 354 | | * | ##### |
| 459 | 355 | | * | ##### |
| 460 | 356 | | ** | ##### |
| 461 | 357 | | * | ##### |
| 453 | 358 | + | | |
| 456 | 359 | + | | |
| 450 | 360 | | * | ##### |
| 452 | 361 | | * | ##### |
| 465 | 362 | | * | ##### |
| 457 | 363 | | ** | ##### |
| 458 | 364 | | * | ##### |
| 436 | 365 | | * | ##### |
| 437 | 366 | + | | |
| 429 | 367 | | * | ##### |
| 418 | 368 | + | | |
| 430 | 369 | | * | ##### |
| 477 | 370 | ++ | | |
| 455 | 371 | | * | ##### |
| 505 | 372 | + | | |
| 487 | 373 | | * | ##### |
| 375 | 374A | | * | ##### |
| 376 | 374B | | * | ##### |
| 425 | 375 | + | | |
| 408 | 376 | + | | |
| 400 | 377 | | * | ##### |
| 402 | 378 | + | | |
| 415 | 379 | ++ | | |
| 424 | 380 | | * | ##### |
| 395 | 381 | ++ | | |
| 397 | 382 | + | * | ##### |
| 393 | 383 | ++ | | |
| 394 | 384 | + | * | ##### |
| 426 | 385 | ++ | | |
| 363 | 386 | ++ | | |
| 362 | 387 | | ** | ##### |
| 422 | 388 | | * | ##### |
| 419 | 389 | + | | |
| 409 | 390 | | * | ##### |
| 412 | 391 | | * | ##### |
| 441 | 392A | | * | # |
| 444 | 392B | | * | ##### |
| 440 | 393 | | * | ##### |
| 423 | 394 | + | | |
| 404 | 395 | ++ | | |
| 484 | 398 | + | | |
| 503 | 399 | + | | |
| 396 | 400 | | * | ##### |
| 407 | 401 | | * | ##### |
| 405 | 402 | | * | ##### |
| 413 | 403 | | * | ##### |
| 388 | 404 | | * | ##### |
| 420 | 405 | | * | #### |
| 383 | 406 | | * | ##### |
| 410 | 407 | | * | ##### |
| 386 | 408 | + | * | ##### |
| 387 | 409 | + | * | ##### |
| 366 | 410 | | * | ##### |
| 367 | 411 | | * | ##### |
| 374 | 412 | | * | ##### |
| 381 | 413 | | * | ##### |
| 447 | 414 | | * | ##### |
| 438 | 415 | | * | ##### |
| 432 | 416 | | * | ##### |
| 399 | 417 | ++ | | |
| 401 | 418 | | * | ##### |
| 507 | 420 | + | ** | ##### |
| 373 | 421 | + | | |
| 359 | 422 | + | * | ##### |
| 360 | 423 | + | * | ##### |
| 492 | 424 | | ** | ##### |
| 385 | 425A | | ** | ##### |
| 369 | 425B | | * | ##### |
| 398 | 428 | | * | ##### |
| 391 | 429 | | * | ##### |
| 392 | 430 | | * | ##### |
| 380 | 431 | | * | ##### |
| 377 | 432 | | * | ##### |
| 406 | 433 | | * | ##### |
| 403 | 434 | | * | ##### |
| 389 | 435 | | * | ##### |
| 379 | 436 | | * | ##### |
| 382 | 437 | | * | ##### |
| 361 | 438 | + | * | ##### |
| 361A | 439 | + | * | ##### |
| 361B | 440 | + | * | ##### |
| 361C | 441 | + | * | ##### |
| 361D | 442 | + | * | ##### |
| 508 | 444 | + | | ##### |
| 476 | 445A | | | ##### |
| 478 | 445B | + | | |
| 428 | 446A | + | | |
| 427 | 446B | | * | ##### |
| 439 | 447A | | * | ##### |
| 442 | 447B | | * | ##### |
| 370 | 448 | ++ | | |
| 371 | 449 | ++ | | |
| 390 | 450 | ++ | | |
| 364 | 451A | | ** | ##### |
| 365 | 451B | | * | ##### |
| 378 | 443 | | * | ##### |
| 433 | 305 | | * | ##### |
| 451 | 426 | + | | |
| 416 | 397 | ++ | | |
| 414 | 396 | ++ | | |

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (I): 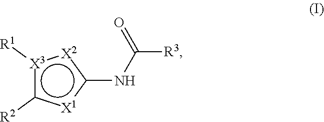
or a pharmaceutically acceptable salt, solvate, clathrate, hydrate, stereoisomer, or tautomer thereof, wherein:
X¹ is CH, S, or N;
X² is N, S, or O;
X³ is C or N;
R¹ and R², together with the atoms to which they are attached, form a C₆-C₁₀ aryl or 5- to 10-membered heteroaryl, wherein the C₆-C₁₀ aryl or 5- to 10-membered heteroaryl is optionally substituted with one or more R^{a};
each R^{a} independently is oxo, halo, cyano, —OH, —NH₂, —C(O)N(R^{b})(R^{c}), —N(R^{b})C(O)(R^{c}), C₁-C₆ alkyl, C₂-C₆ alkenyl, C₂-C₆ alkynyl, C₁-C₆ haloalkyl, C₃-C₁₀ cycloalkyl, C₄-C₁₀ cycloalkenyl, C₆-C₁₀ aryl, 5- to 10-membered heteroaryl, 3- to 10-membered heterocycloalkyl, or 4- to 10-membered heterocycloalkenyl, wherein the C₁-C₆ alkyl, C₂-C₆ alkenyl, C₂-C₆ alkynyl, C₁-C₆ haloalkyl, C₃-C₁₀ cycloalkyl, C₄-C₁₀ cycloalkenyl, C₆-C₁₀ aryl, 5- to 10-membered heteroaryl, 3- to 10-membered heterocycloalkyl, or 4- to 10-membered heterocycloalkenyl is optionally substituted with one or more R^{a1};
each R^{b} and R^{c} independently is H, C₁-C₆ alkyl, C₃-C₁₀ cycloalkyl, or 3- to 10-membered heterocycloalkyl, wherein the C₁-C₆ alkyl, C₃-C₁₀ cycloalkyl, or 3- to 10-membered heterocycloalkyl is optionally substituted with one or more —OH or C₁-C₆ haloalkyl; or
R^{b} and R^{c}, together with the atoms to which they are attached, form a 3- to 10-membered heterocycloalkyl, wherein the 3- to 10-membered heterocycloalkyl is optionally substituted with one or more —OH, —O(C₁-C₆ haloalkyl), —O(C₁-C₆ alkyl), —C(O)(O—(C₁-C₆ alkyl)), C₁-C₆ alkyl, or —N(C₁-C₆ alkyl)₂;
each R^{a1} independently is oxo, halo, cyano, —OH, —O(C₃-C₁₀ cycloalkyl), —NH₂, —NH(C₁-C₆ alkyl), —N(C₁-C₆ alkyl)₂, —C(O)OH, —C(O)O(C₁-C₆ alkyl), C₁-C₆ alkyl, C₂-C₆ alkenyl, C₂-C₆ alkynyl, C₁-C₆ haloalkyl, C₁-C₆ alkoxy, C₁-C₆ haloalkoxy, —O—(C₃-C₁₀ cycloalkyl), C₃-C₁₀ cycloalkyl, 3- to 10-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl is optionally substituted with one or more R^{a2};
each R^{a2} independently is oxo, halo, cyano, —OH, or —NH₂;
R³ is C₆-C₁₀ aryl or 6- to 10-membered heteroaryl, wherein the C₆-C₁₀ aryl or 6- to 10-membered heteroaryl is substituted with one or more R^{3a};
each R^{3a} independently is halo, cyano, —OH, —NH₂, C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, —O(C₆-C₁₀ aryl), C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, —O(C₆-C₁₀ aryl), C₆-C₁₀ aryl, or 5- to 10-membered heteroaryl is optionally substituted with one or more R^{3a1}; and
each R^{3a1} independently is oxo, halo, cyano, —NH₂, —NH—C(O)O(C₁-C₆ alkyl), —C(O)(C₁-C₆ alkyl), —C(O)NH₂, —C(O)(O—(C₁-C₆ alkyl)), C₁-C₆ alkyl optionally substituted with one or more C₃-C₁₀ cycloalkyl, C₂-C₆ alkenyl, C₂-C₆ alkynyl, C₁-C₆ haloalkyl, C₁-C₆ alkoxy, —O(C₁-C₆ haloalkyl), C₃-C₁₀ cycloalkyl, C₆-C₁₀ aryl, or 5- to 10-membered heteroaryl;
wherein, when R^{3a} is halo and R¹ and R², together with the atoms to which they are attached, form a 6-membered heteroaryl then R^{a} is not —CF₃.

2. A compound of Formula (I): 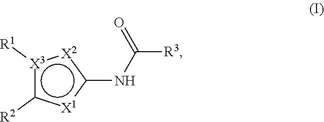
or a pharmaceutically acceptable salt, solvate, clathrate, hydrate, stereoisomer, or tautomer thereof, wherein:
X¹ is CH, S, or N;
X² is N, S, or O;
X³ is C or N;
R¹ and R², together with the atoms to which they are attached, form a C₆-C₁₀ aryl or 5- to 10-membered heteroaryl, wherein the C₆-C₁₀ aryl or 5- to 10-membered heteroaryl is optionally substituted with one or more R^{a};
each R^{a} independently is oxo, halo, cyano, —OH, —NH₂, —C(O)N(R^{b})(R^{c}), —N(R^{b})C(O)(R^{c}), C₁-C₆ alkyl, C₂-C₆ alkenyl, C₂-C₆ alkynyl, C₁-C₆ haloalkyl, C₃-C₁₀ cycloalkyl, C₆-C₁₀ aryl, 5- to 10-membered heteroaryl, or 3- to 10-membered heterocycloalkyl, wherein the C₁-C₆ alkyl, C₂-C₆ alkenyl, C₂-C₆ alkynyl, C₁-C₆ haloalkyl, C₃-C₁₀ cycloalkyl, C₆-C₁₀ aryl, 5- to 10-membered heteroaryl, or 3- to 10-membered heterocycloalkyl is optionally substituted with one or more R^{a1};
each R^{b} and R^{c} independently is H, C₁-C₆ alkyl, C₃-C₁₀ cycloalkyl, or 3- to 10-membered heterocycloalkyl, wherein the C₁-C₆ alkyl, C₃-C₁₀ cycloalkyl, or 3- to 10-membered heterocycloalkyl is optionally substituted with one or more —OH or C₁-C₆ haloalkyl; or
R^{b} and R^{c}, together with the atoms to which they are attached, form a 3- to 10-membered heterocycloalkyl, wherein the 3- to 10-membered heterocycloalkyl is optionally substituted with one or more —OH, —O(C₁-C₆ haloalkyl), —O(C₁-C₆ alkyl), —C(O)(O—(C₁-C₆ alkyl)), C₁-C₆ alkyl, or —N(C₁-C₆ alkyl)₂;
each R^{a1} independently is oxo, halo, cyano, —OH, —O(C₃-C₁₀ cycloalkyl), —NH₂, —NH(C₁-C₆ alkyl), —N(C₁-C₆ alkyl)₂, —C(O)OH, —C(O)O(C₁-C₆ alkyl), C₁-C₆ alkyl, C₂-C₆ alkenyl, C₂-C₆ alkynyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl is optionally substituted with one or more R^{a2};
each R^{a2} independently is oxo, halo, cyano, —OH, or —NH₂;
R³ is C₆-C₁₀ aryl or 6- to 10-membered heteroaryl, wherein the C₆-C₁₀ aryl or 6- to 10-membered heteroaryl is substituted with one or more R^{3a};
each R^{3a} independently is halo, cyano, —OH, —NH₂, C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ haloalkyl, C₃-C₁₀ cycloalkyl, 3- to 10-membered heterocycloalkyl, C₆-C₁₀ aryl, or 5- to 10-membered heteroaryl is optionally substituted with one or more R^{3a1}; and
each R^{3a1} independently is oxo, halo, cyano, —C(O)(C₁-C₆ alkyl), —C(O)NH₂, —C(O)(0-(C₁-C₆ alkyl)), C₁-C₆ alkyl optionally substituted with one or more C₃-C₁₀ cycloalkyl, C₂-C₆ alkenyl, C₂-C₆ alkynyl, C₁-C₆ haloalkyl, C₁-C₆ alkoxy, —O(C₁-C₆ haloalkyl), C₃-C₁₀ cycloalkyl, C₆-C₁₀ aryl, or 5- to 10-membered heteroaryl;
wherein, when R^{3a} is halo and R¹ and R², together with the atoms to which they are attached, form a 6-membered heteroaryl then R^{a} is not —CF₃.

3. The compound of claim 1 or claim 2, wherein:
(ii) X¹ is CH or N; and R¹ and R², together with the atoms to which they are attached, form a pyridine optionally substituted with one or more R^{a}; or
(iii) X² is N or O; and R¹ and R², together with the atoms to which they are attached, form a pyridine optionally substituted with one or more R^{a}; or
(iv) when R¹ and R², together with the atoms to which they are attached, form a pyridine optionally substituted with one or more R^{a}, then X¹ is not S; or
(v) when R¹ and R², together with the atoms to which they are attached, form a pyridine optionally substituted with one or more R^{a}, then X² is not S; or
(vi) when R¹ and R², together with the atoms to which they are attached, form a pyridine optionally substituted with one or more R^{a}, then 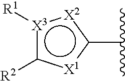
is not 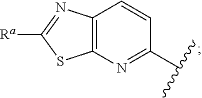
or
(vii) when R^{a} is 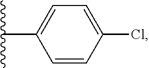 [SMILES: *c1ccc(Cl)cc1]
then R³ is not 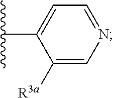
or
(viii) when R^{a} is 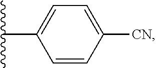 [SMILES: *c1ccc(C#N)cc1]
then R³ is not 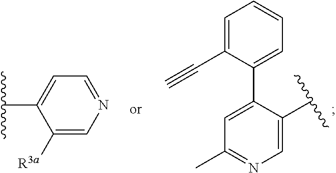
or
(ix) when R¹ and R², together with the atoms to which they are attached, form a pyrazine substituted with one or more R^{a}, and R^{a} is phenyl substituted with one or more R^{a1}, then R^{a1} is not —Cl, —F, —CN, —O(CH₃), —O(CF₃), —O(CHF₂), —CH₃, or cyclopropyl; or
(x) when R¹ and R², together with the atoms to which they are attached, form a pyrazine substituted with one or more R^{a}, and R^{a} is pyridine substituted with one or more R^{a1}, then R^{a1} is not —Cl, —CN, —CF₃, —O(CH₃), —O(CHF₂), cyclopropyl, —C(CH₃)₂(CN), or —C(CH₃)₂(OH); or
(xi) when R¹ and R², together with the atoms to which they are attached, form a pyrazine substituted with one or more R^{a}, then R^{a} is not 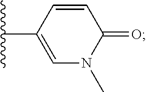 [SMILES: *c1ccc(=O)n(C)c1]
or
(xii) the compound of Formula (I) is not 5′-methoxy-2′,6-dimethyl-N-(6-(tetrahydrofuran-3-yl)thiazolo[4,5-b]pyrazin-2-yl)-[4,4′-bipyridine]-3-carboxamide.

4. The compound of any one of the preceding claims, wherein X¹ is CH or N.

5. The compound of any one of the preceding claims, wherein:
each R^{a} independently is oxo, halo, —C(O)N(R^{b})(R^{c}), —N(R^{b})C(O)(R^{c}), C₁-C₆ alkyl, C₁-C₆ haloalkyl, C₃-C₁₀ cycloalkyl, C₆-C₁₀ aryl, 5- to 10-membered heteroaryl, or 3- to 10-membered heterocycloalkyl, wherein the C₁-C₆ alkyl, C₁-C₆ haloalkyl, C₃-C₁₀ cycloalkyl, C₆-C₁₀ aryl, 5- to 10-membered heteroaryl, or 3- to 10-membered heterocycloalkyl is optionally substituted with one or more R^{a1};
each R^{a1} independently is oxo, halo, cyano, —OH, —O(C₃-C₁₀ cycloalkyl), —NH(C₁-C₆ alkyl), —N(C₁-C₆ alkyl)₂, —C(O)O(C₁-C₆ alkyl), C₁-C₆ alkyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl, wherein the C₁-C₆ alkyl, C₁-C₆ haloalkyl, C₁-C₆ alkoxy, C₃-C₁₀ cycloalkyl, 3- to 10-membered heterocycloalkyl, C₆-C₁₀ aryl, or 5- to 10-membered heteroaryl is optionally substituted with one or more R^{a2};
each R^{a2} independently is cyano or —OH;
each R^{3a} independently is cyano, C₁-C₆ alkyl, C₁-C₆ haloalkyl, 3- to 10-membered heterocycloalkyl, C₆-C₁₀ aryl, or 5- to 10-membered heteroaryl, wherein the C₁-C₆ alkyl, C₁-C₆ haloalkyl, C₃-C₁₀ cycloalkyl, 3- to 10-membered heterocycloalkyl, C₆-C₁₀ aryl, or 5- to 10-membered heteroaryl is optionally substituted with one or more R^{3a1}; and
each R^{3a1} independently is oxo, halo, cyano, —C(O)(C₁-C₆ alkyl), —C(O)NH₂, —C(O)(0-(C₁-C₆ alkyl)), C₁-C₆ alkyl optionally substituted with one or more C₃-C₁₀ cycloalkyl, C₁-C₆ alkoxy, —O(C₁-C₆ haloalkyl), C₃-C₁₀ cycloalkyl, or 5- to 10-membered heteroaryl.

6. The compound of any one of the preceding claims, wherein R¹ and R², together with the atoms to which they are attached, form a C₆ aryl or 6-membered heteroaryl optionally substituted with one or more R^{a}.

7. The compound of any one of the preceding claims, wherein R¹ and R², together with the atoms to which they are attached, form a 5-membered heteroaryl optionally substituted with one or more R^{a}.

8. The compound of any one of the preceding claims, wherein R¹ and R², together with the atoms to which they are attached, form pyrazinyl, pyridinyl, or diazolyl, wherein the pyrazinyl, pyridinyl, or diazolyl is optionally substituted with one or more R^{a}.

9. The compound of any one of the preceding claims, wherein each R^{a} independently is: oxo, —Cl, —Br, —CH₃, —CF₃,

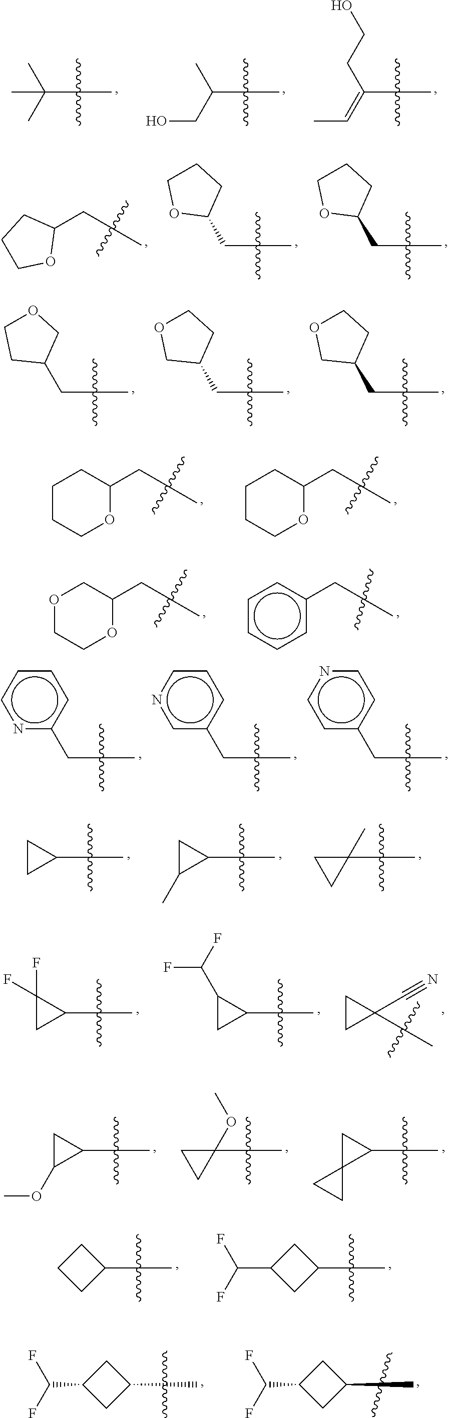

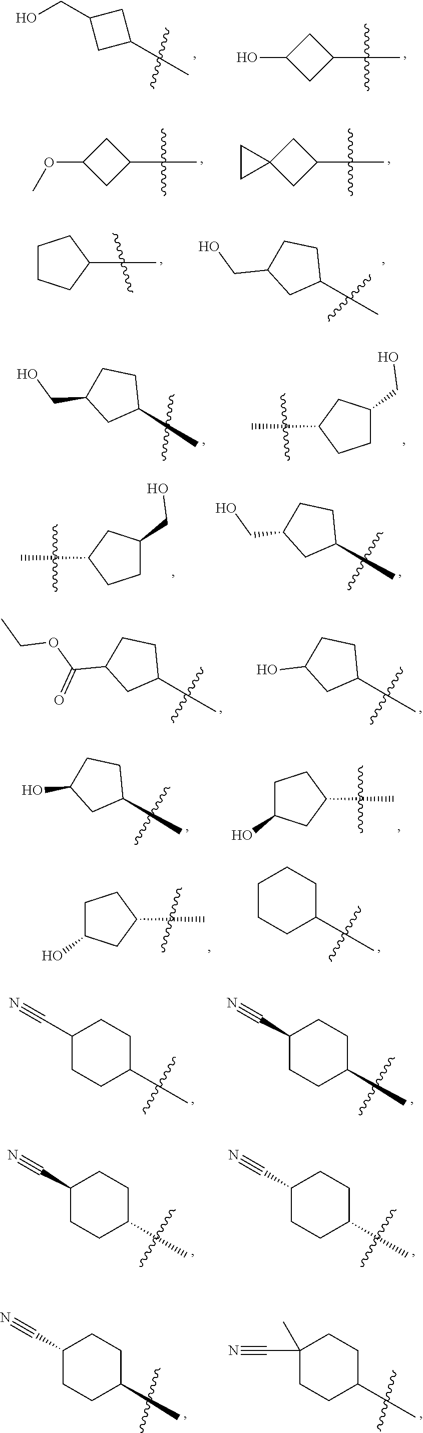

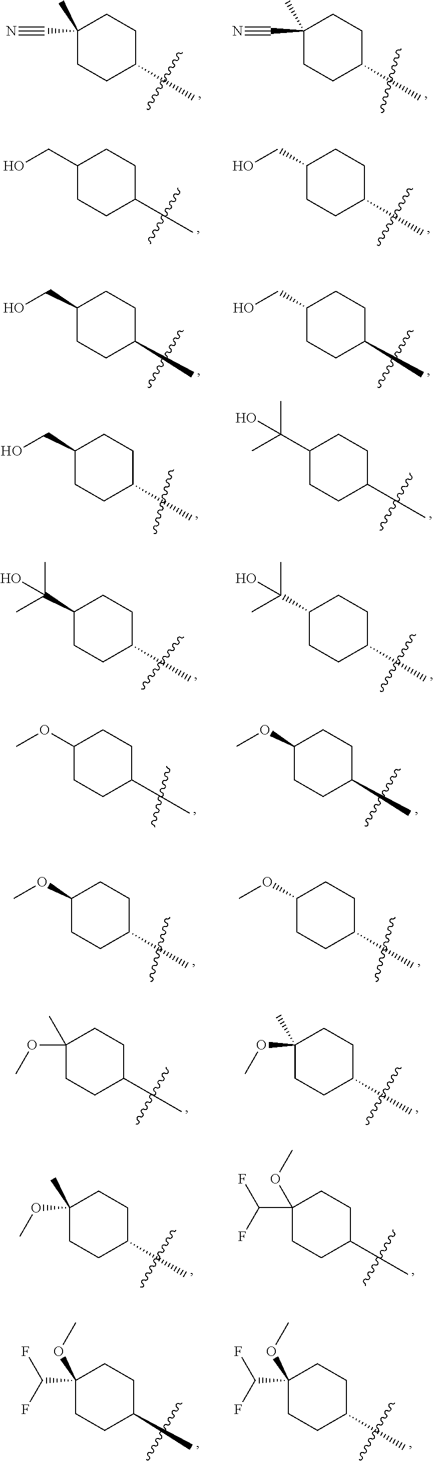

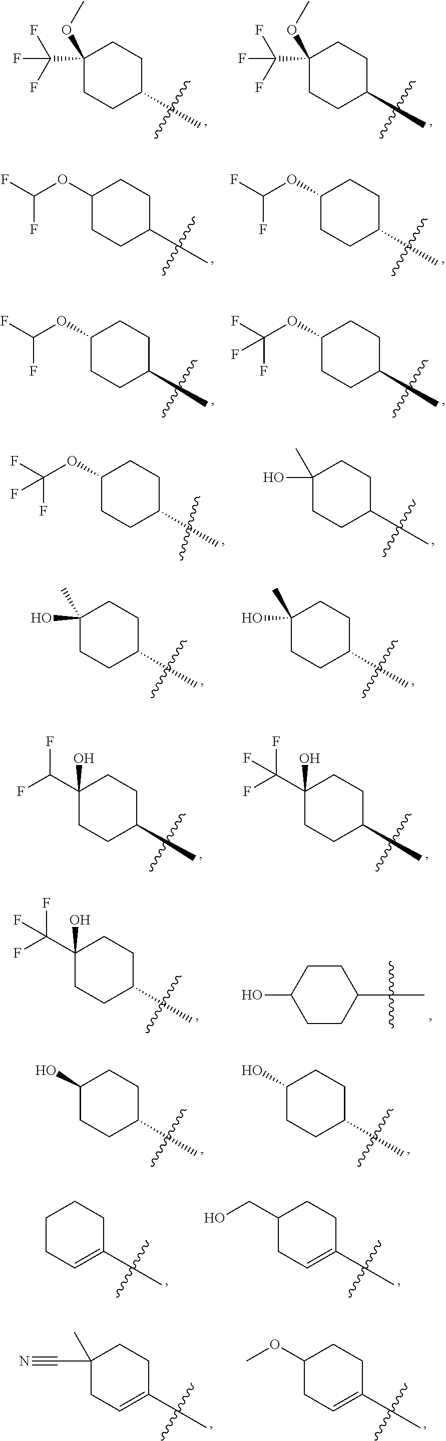

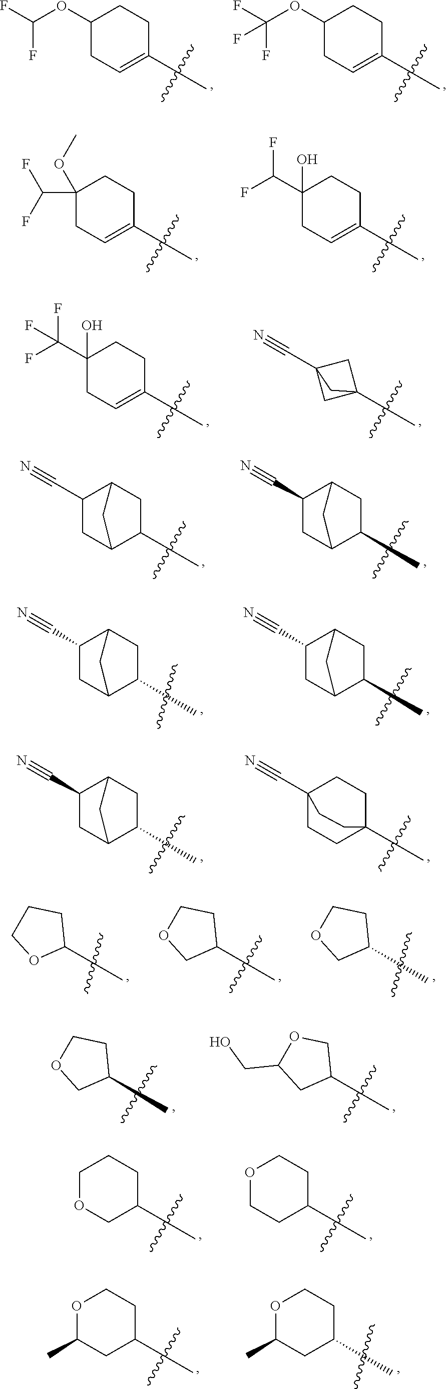

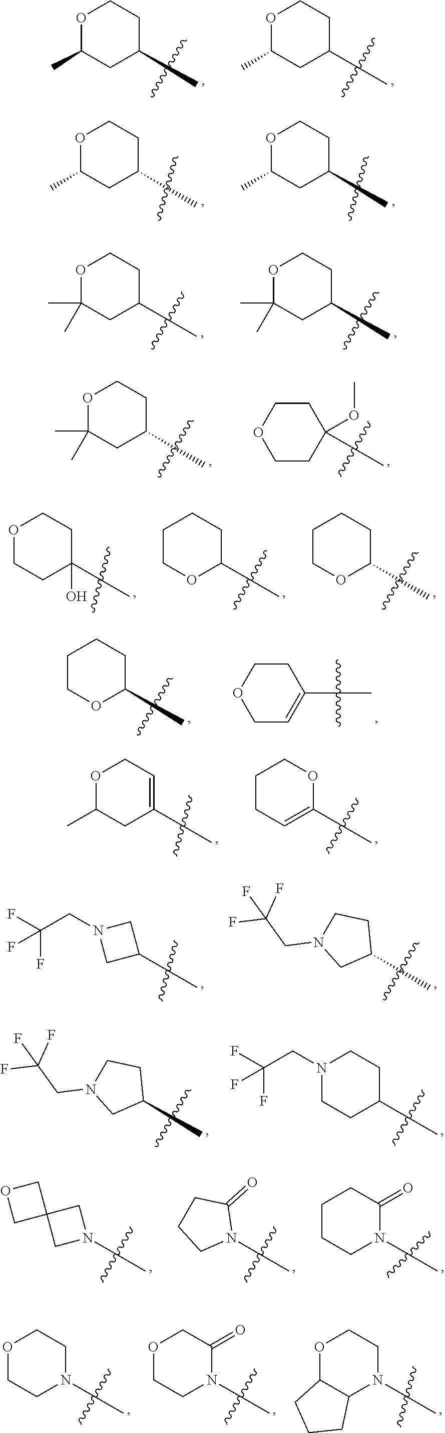

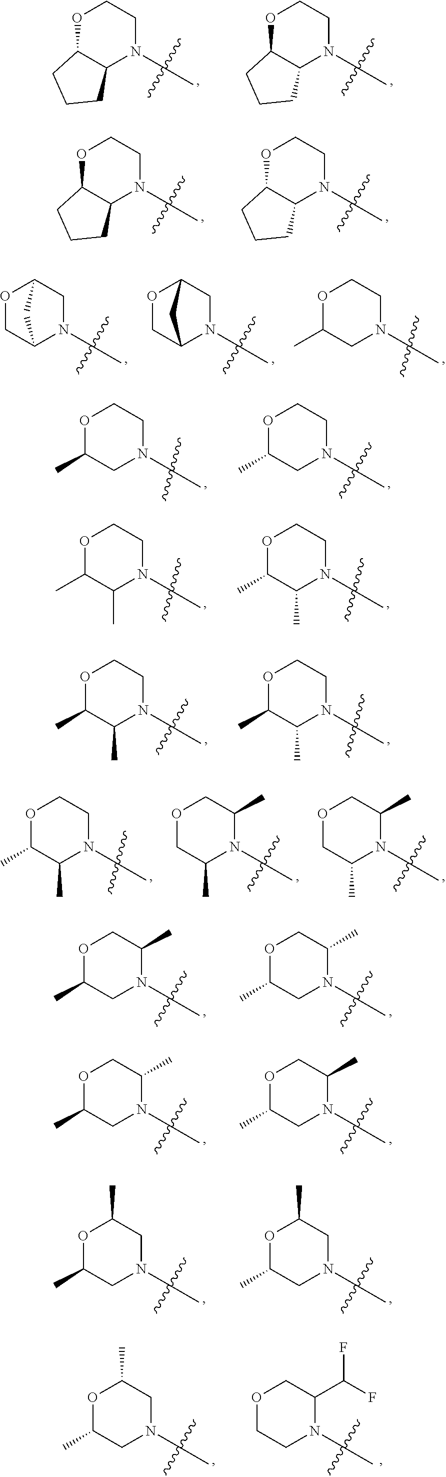

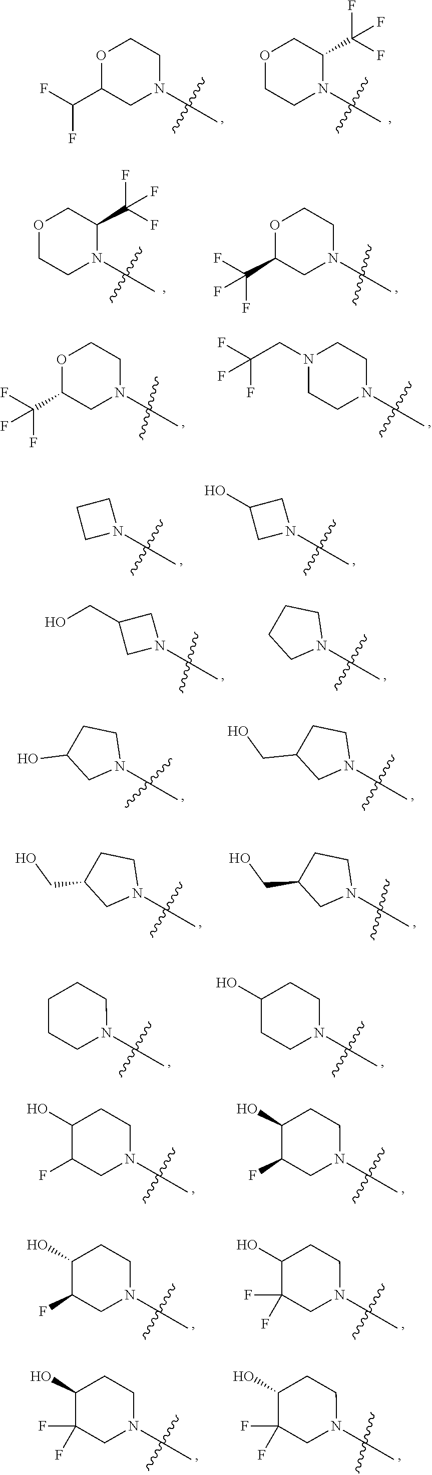

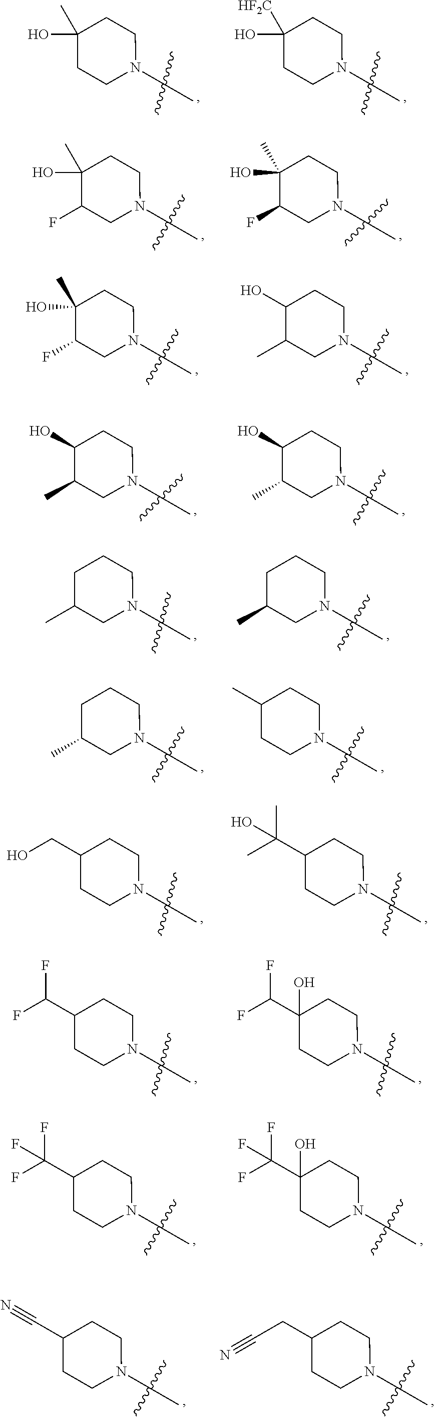

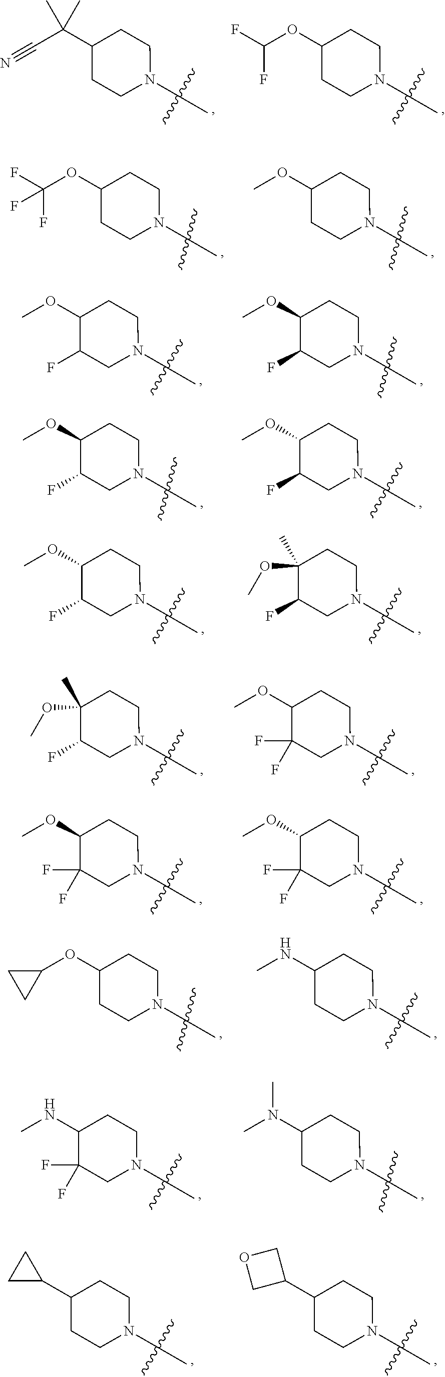

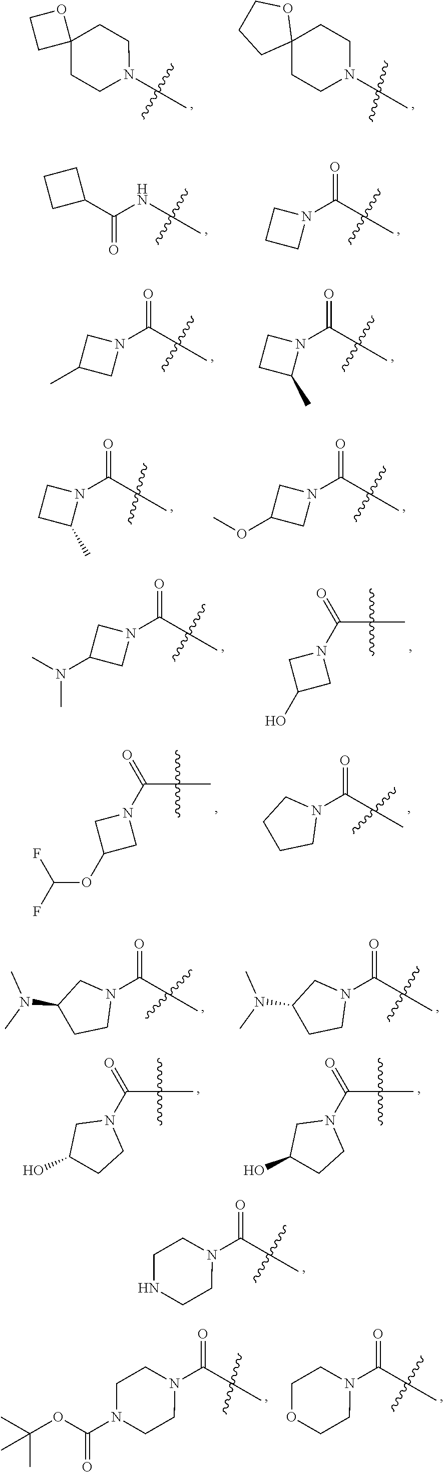

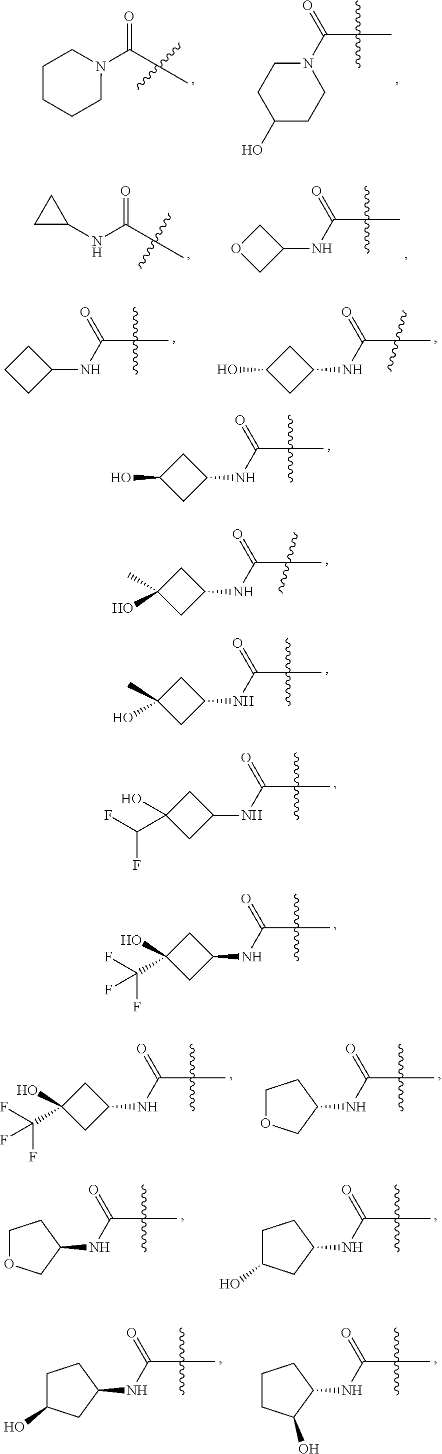

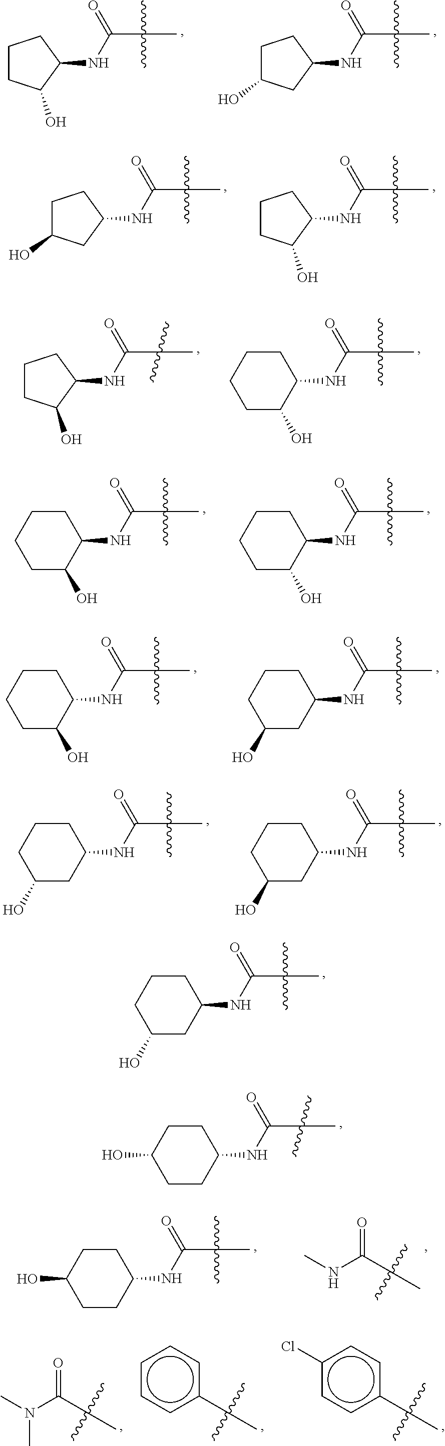

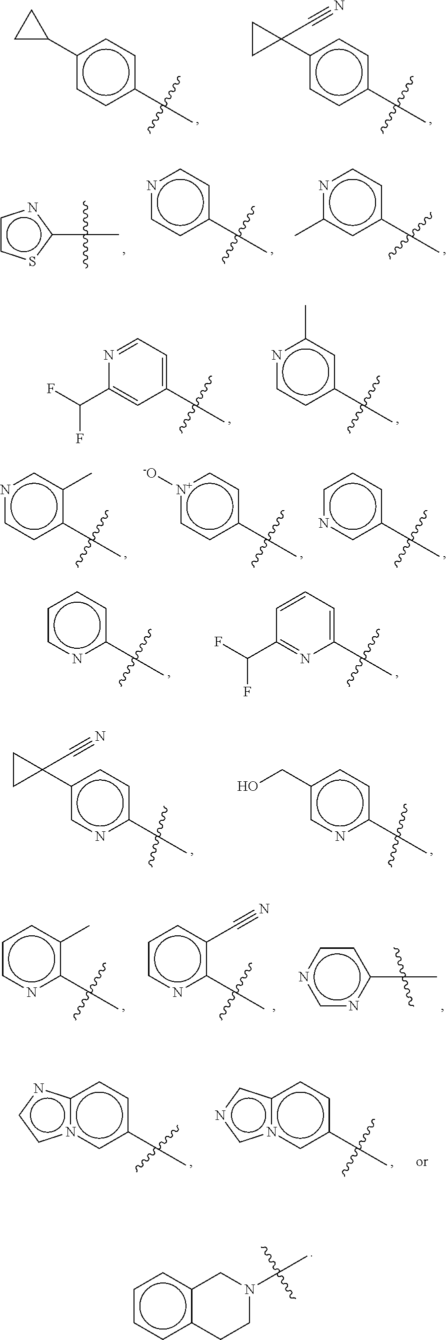

10. The compound of any one of the preceding claims, wherein each R^{b} and R^{c} independently is H, methyl, ethyl, cyclobutyl, cyclopropyl,

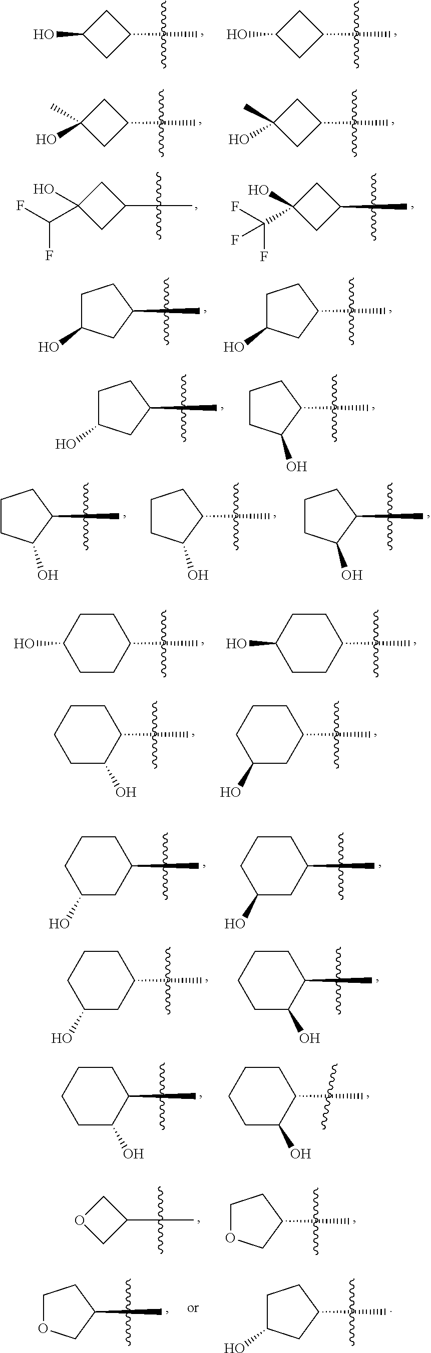

11. The compound of any one of the preceding claims, wherein R^{b} and R^{c}, together with the atoms to which they are attached, form a heterocycloalkyl selected from:

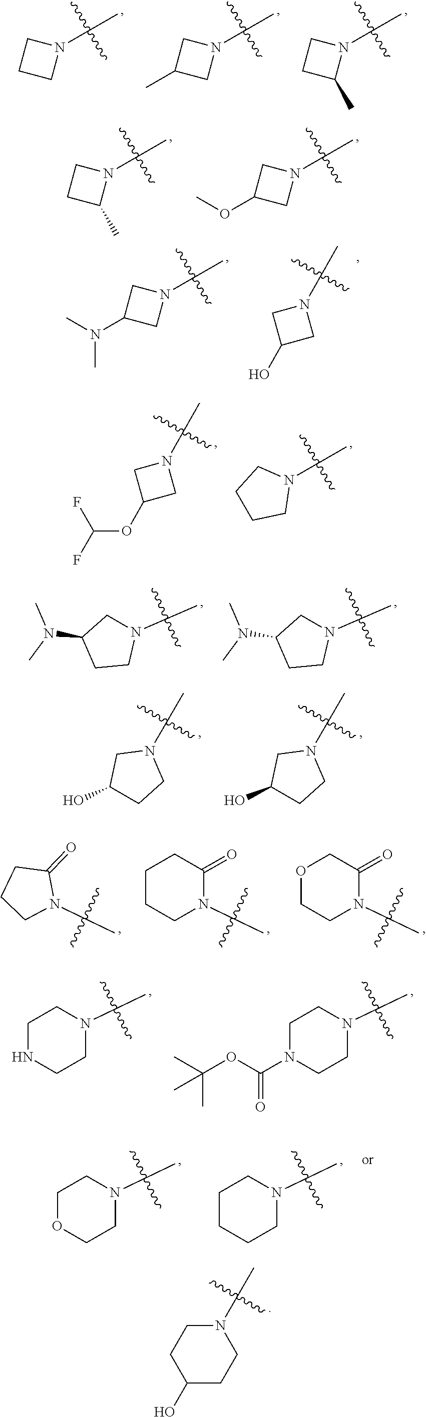

12. The compound of any one of the preceding claims, wherein R³ is:

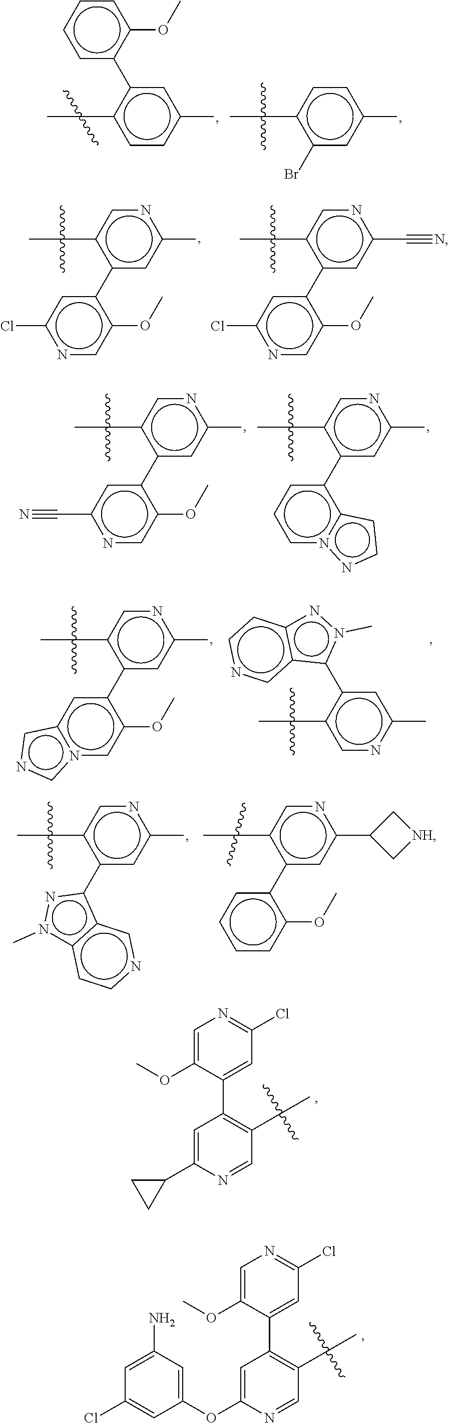

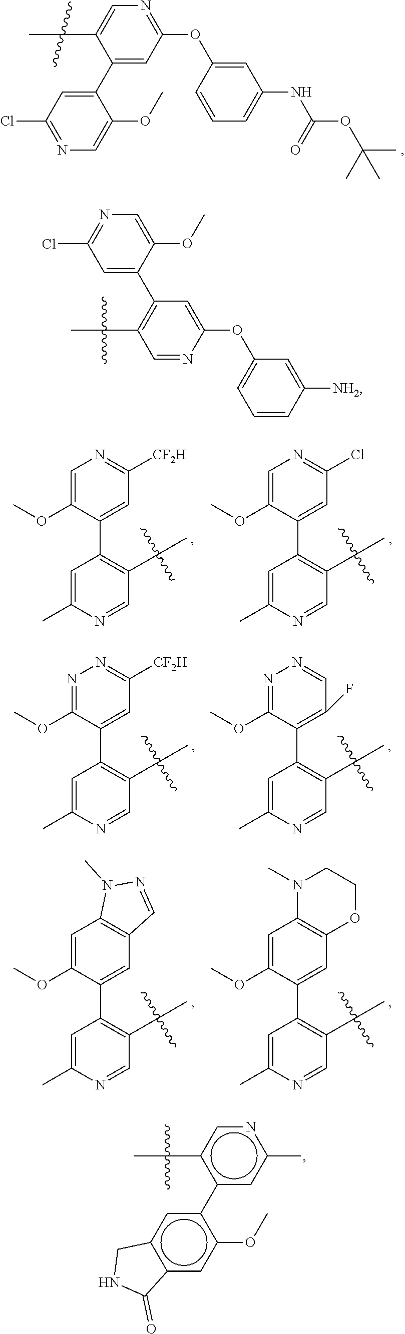

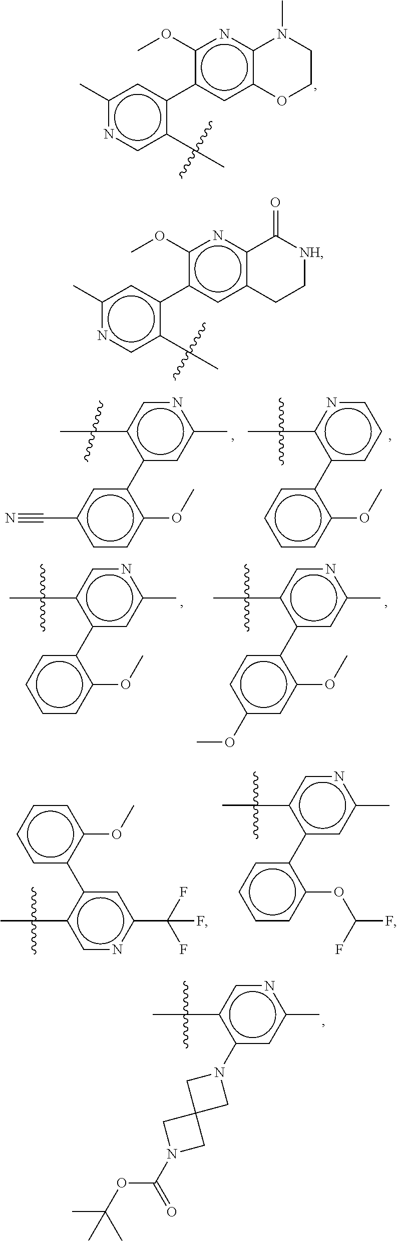

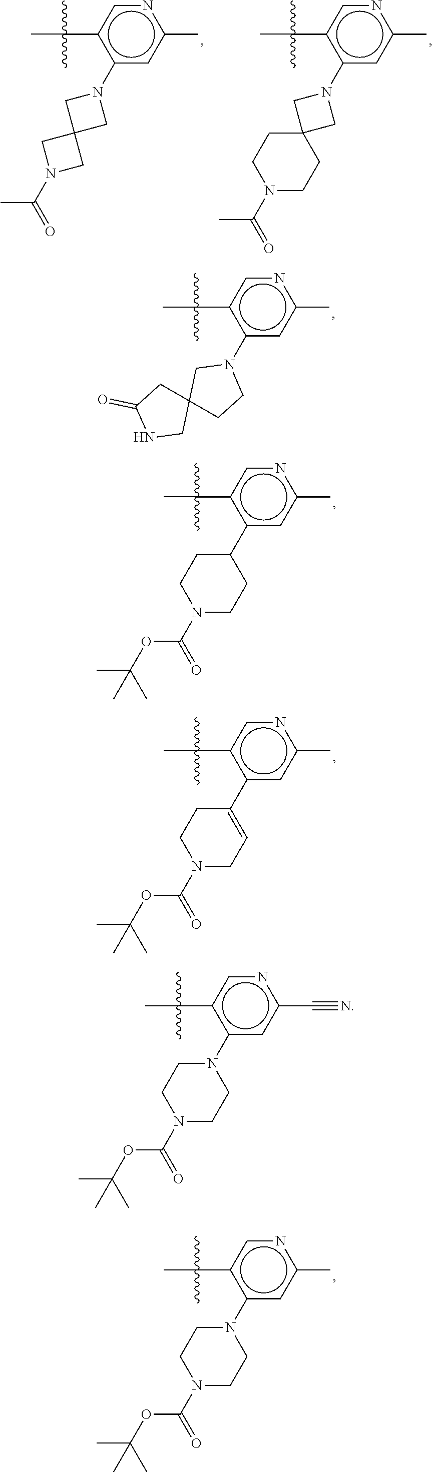

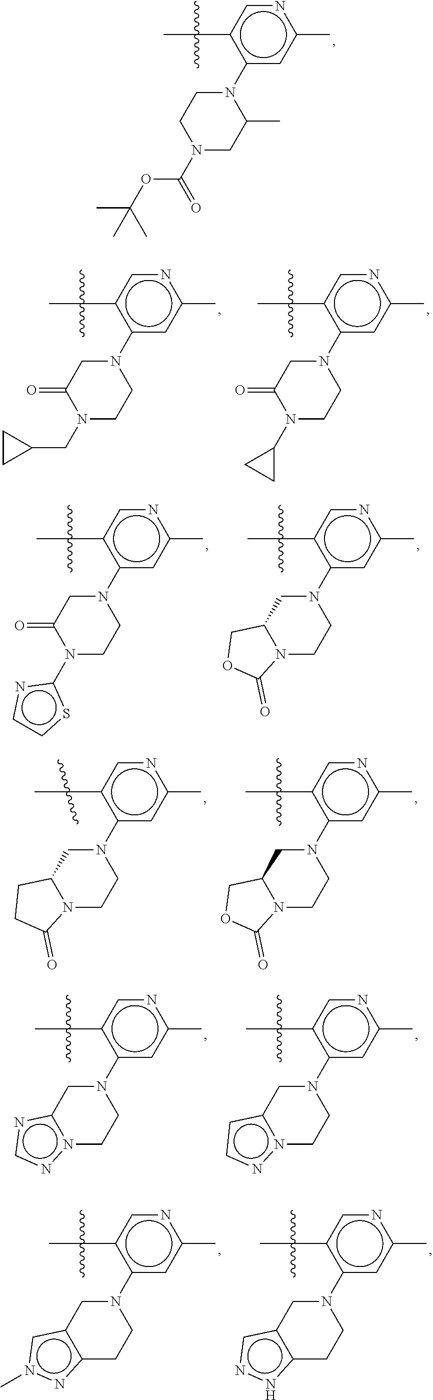

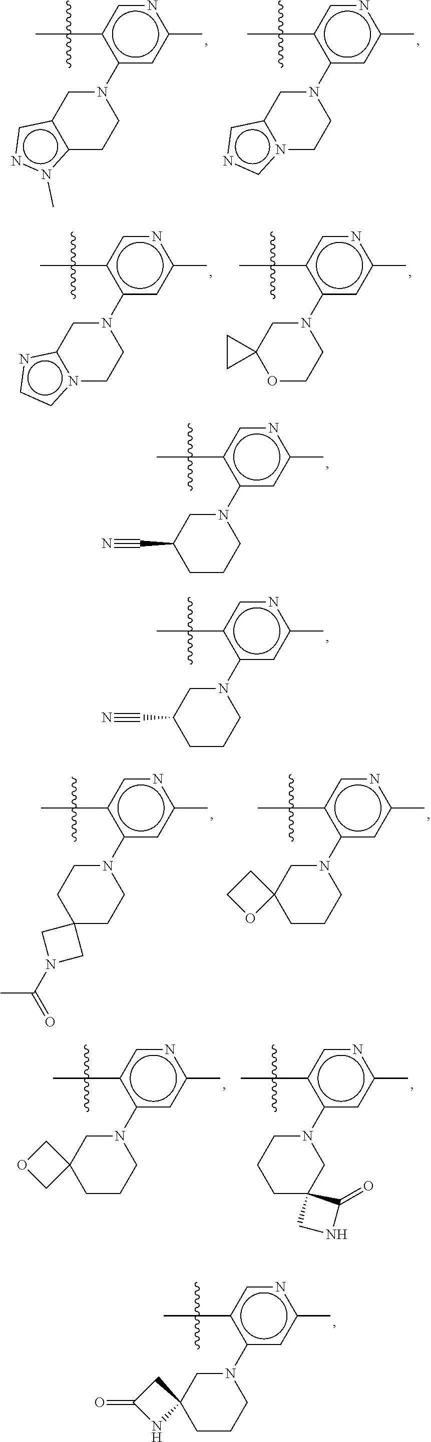

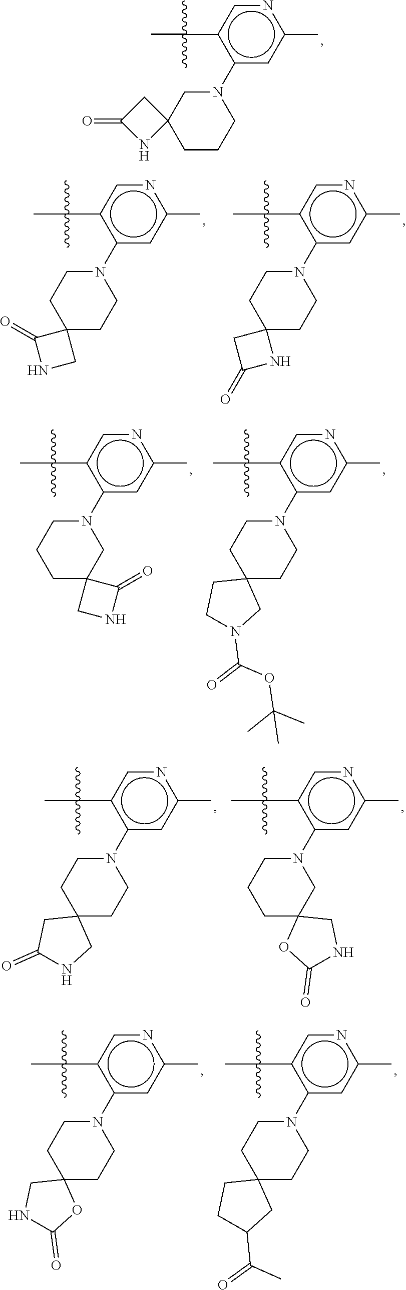

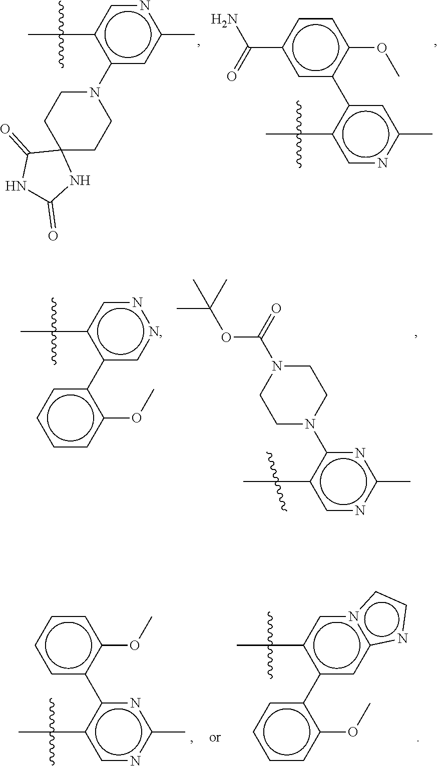

13. The compound of any one of the preceding claims, wherein each R^{a1} independently is oxo, —OH, —CH₂OH, —CHF₂, —CF₃, —F, —Cl, —CN, —OCH₃, —OCHF₂, —OCF₃, —NHCH₃, —N(CH₃)₂, —CH₃, —CH₂CF₃, —CH₂CN,

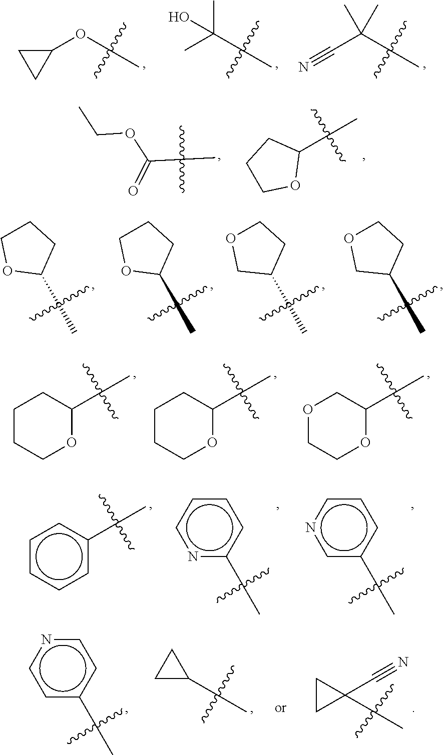

14. The compound of any one of the preceding claims, wherein each R^{3a} independently is —Br, —CH₃, —CF₃, —CN,

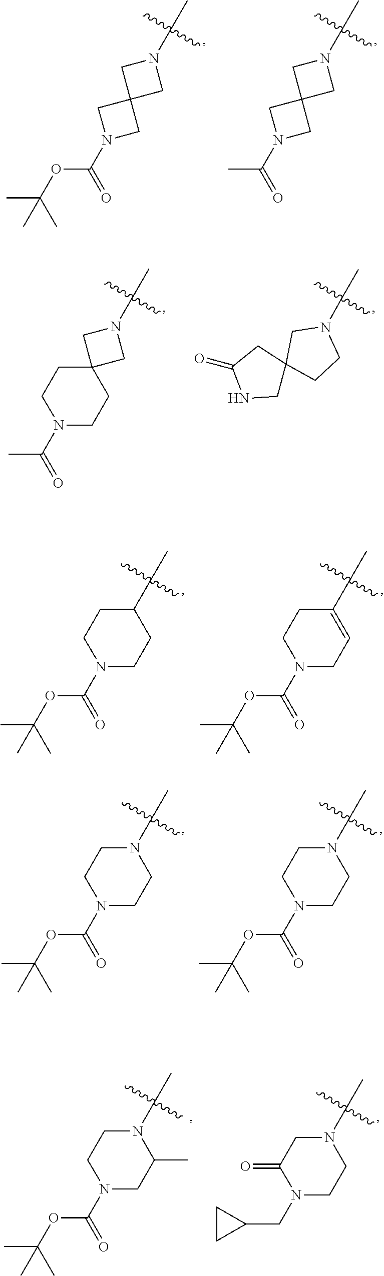

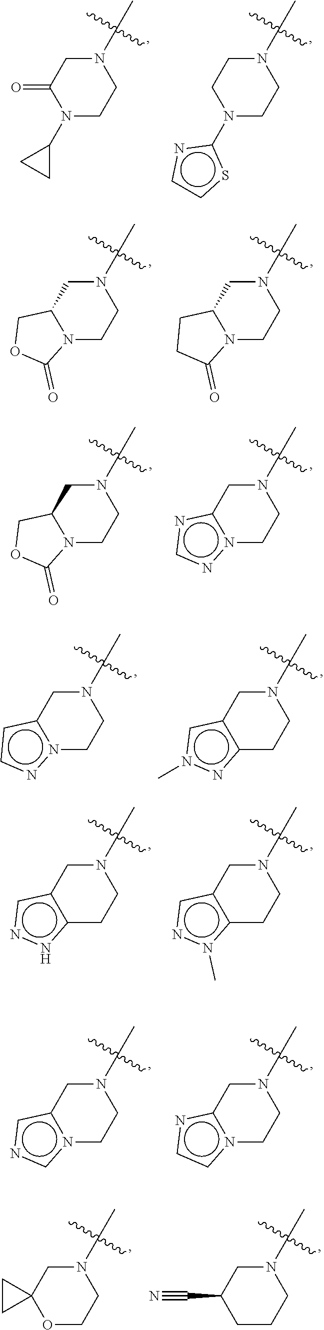

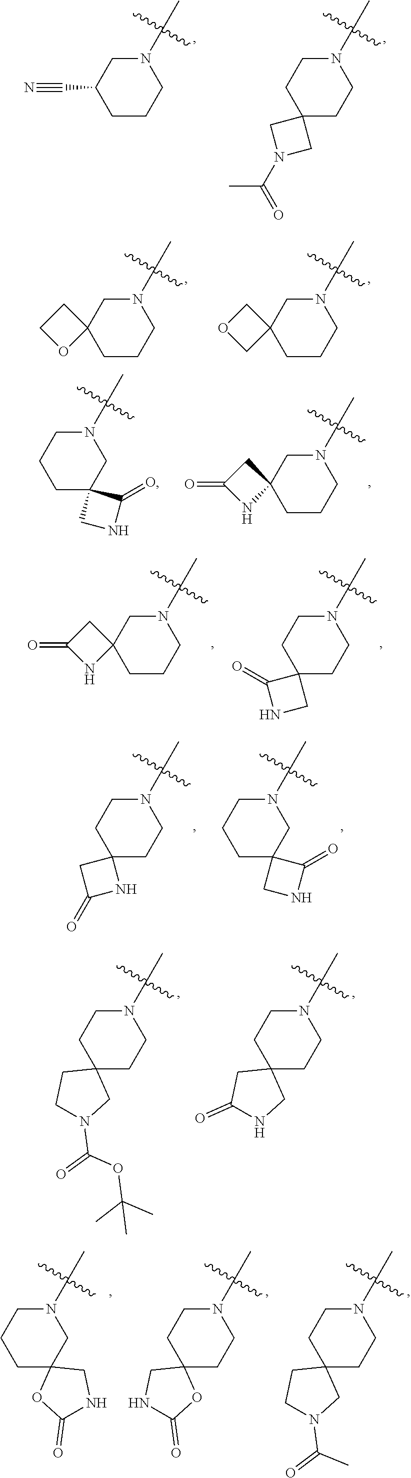

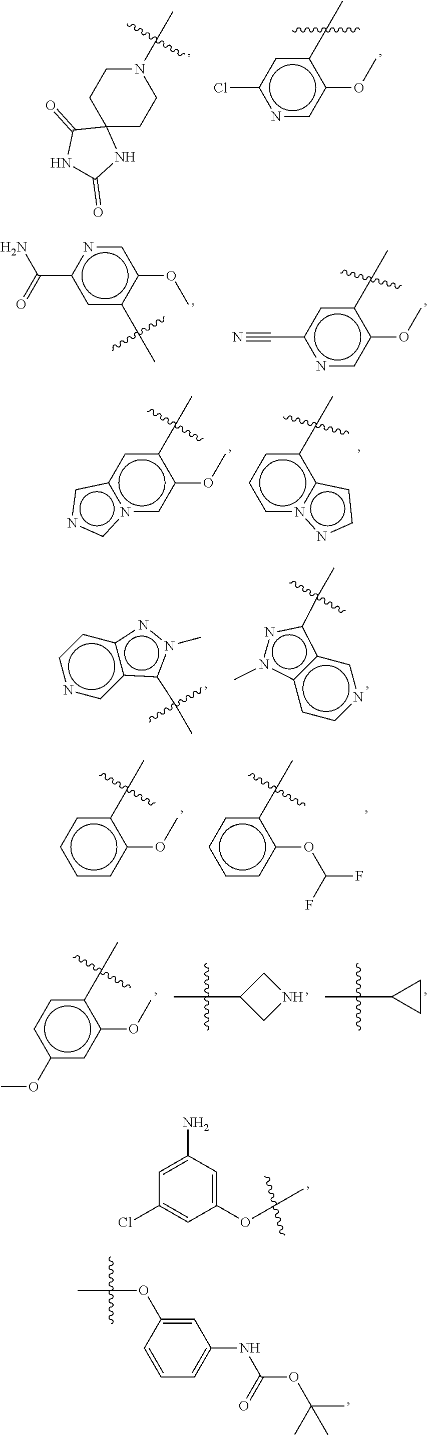

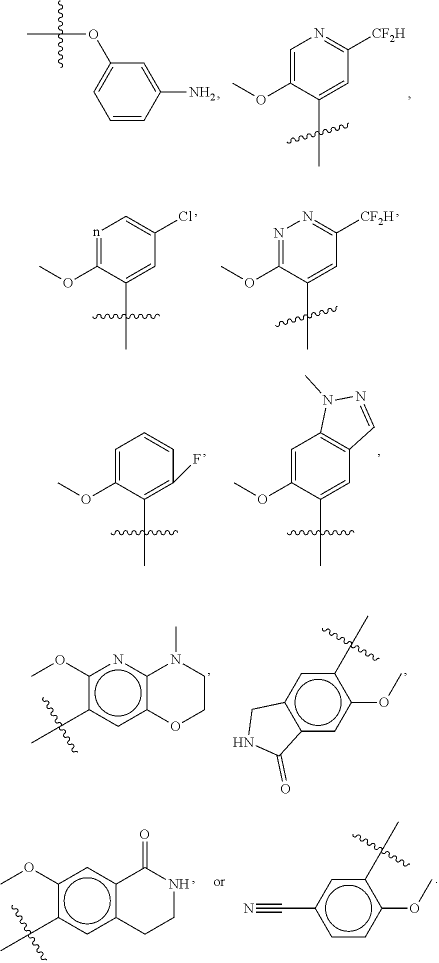

15. The compound of any one of the preceding claims, wherein each R^{3a1} independently is oxo, —Cl, —CH₃, —OCH₃, —CF₂H, —NH₂, —NHC(O)OC(CH₃)₃, —OCHF₂, —C(O)OC(CH₃)₃, —C(O)CH₃, —C(O)NH₂, —CN,

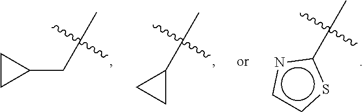

16. The compound of any one of the preceding claims, wherein each R² independently is cyano.

17. The compound of any one of the preceding claims, wherein the compound is of Formula (I-A), (I-B), or (I-C): 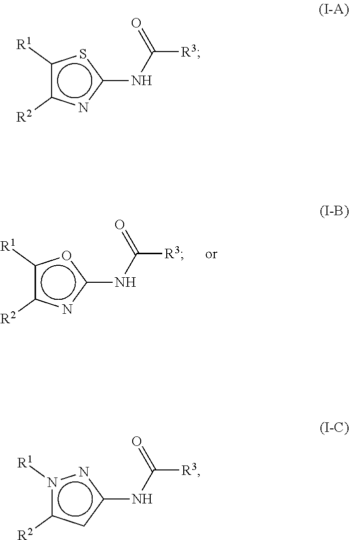
or a pharmaceutically acceptable salt, solvate, clathrate, hydrate, stereoisomer, or tautomer thereof.

18. The compound of any one of claims 1-6, wherein the compound is of Formula (I-D), (I-E), (I-F), (I-G), (I-H), (I-I), (I-J), (I-K), or (I-L): 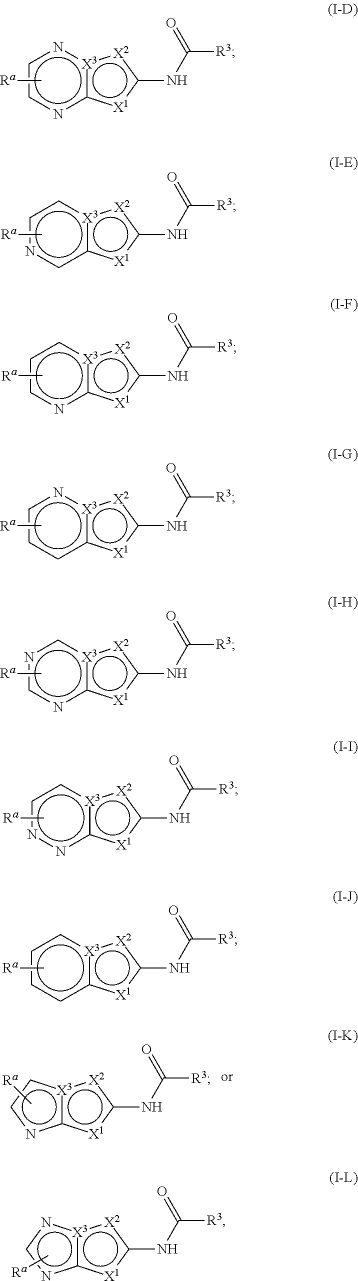
or a pharmaceutically acceptable salt, solvate, clathrate, hydrate, stereoisomer, or tautomer thereof.

19. The compound of any one of claims 1-6, wherein the compound is of Formula (I-F′): 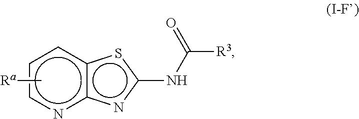
or a pharmaceutically acceptable salt, solvate, clathrate, hydrate, stereoisomer, or tautomer thereof.

20. The compound of any one of claims 1-6, wherein the compound is of Formula (I-M), (I—), (I-O), (I-P), (I-Q), (I-R), (I-S), (I-T), (I-U), or (I-V): 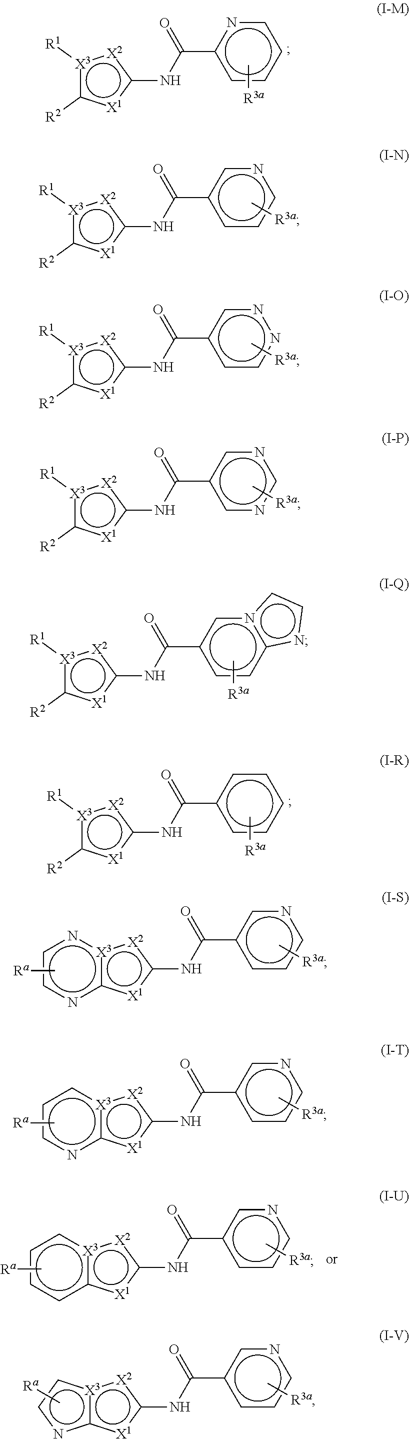
or a pharmaceutically acceptable salt, solvate, enantiomer, diastereomer, racemate or mixture thereof.

21. The compound of any one of claims 1-6, wherein the compound is of Formula (I-T′): 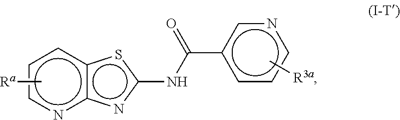
or a pharmaceutically acceptable salt, solvate, enantiomer, diastereomer, racemate or mixture thereof.

22. The compound of any one of claims 1-6, wherein the compound is of Formula (I-W): 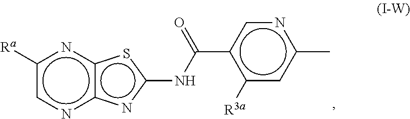
or a pharmaceutically acceptable salt, solvate, enantiomer, diastereomer, racemate or mixture thereof.

23. The compound of any one of the preceding claims, wherein the compound is selected from a compound described in Tables 1-4, or a pharmaceutically acceptable salt thereof.

24. The compound of any one of the preceding claims, wherein the compound is selected from Compound Nos. 1, 10, 24, 25, 59, 207, 211, 212, 224, 235, 238, 239, 244, 3, 12, 280, 282, 288, 308, 359, 360, 361B, 368, 377, 386, 391, 394, 403, 427, 432, 447, 451A, 454, 462, 464, 467, 486, 489, 498, 502, 504, 506, 514, 518, 520, 521, and 522, or a pharmaceutically acceptable salt thereof.

25. A compound obtainable by, or obtained by, a method described herein;
optionally, the method comprises one or more steps described in Schemes 1-11.

26. A pharmaceutical composition comprising the compound of any one of the preceding claims or a pharmaceutically acceptable salt thereof, and a pharmaceutically acceptable diluent or carrier.

27. The pharmaceutical composition of claim 26, wherein the compound is selected from a compound described in Tables 1-4.

28. A method of modulating DNA polymerase Θ activity, comprising contacting a cell with a compound of any one of claims 1-25 or a pharmaceutical composition of claim 26 or claim 27.

29. A method of treating or preventing a disease or disorder in a subject in need thereof, comprising administering to the subject a compound of any one of claims 1-25 or pharmaceutical composition of claim 26 or claim 27.

30. The compound of any one of claims 1-25 or pharmaceutical composition of claim 26 or claim 27 for use in modulating DNA polymerase Θ activity.

31. The compound of any one of claims 1-25 or pharmaceutical composition of claim 26 or claim 27 for use in treating or preventing a disease or disorder.

32. Use of the compound of any one of claims 1-25 in the manufacture of a medicament for modulating DNA polymerase Θ activity.

33. Use of the compound of any one of claims 1-25 in the manufacture of a medicament for treating or preventing a disease or disorder.

34. The method, compound, pharmaceutical composition, or use of any one of claims 28-33 wherein the disease or disorder is associated with an implicated DNA polymerase Θ activity.

35. The method, compound, pharmaceutical composition, or use of any one of claims 28-34, wherein the disease or disorder is cancer.
